(19) United States
(12) Patent Application Publication
(10) Pub. No.: US 2023/0135894 A1
(43) Pub. Date: May 4, 2023

(54) MK2 DEGRADERS AND USES THEREOF

(71) Applicant: Kymera Therapeutics, Inc., Watertown, MA (US)

(72) Inventors: Bin YANG, Lexington, MA (US); Matthew M. WEISS, Boston, MA (US); Xiao ZHU, Winchester, MA (US); Huijun DONG, Watertown, MA (US); Isaac MARX, Watertown, MA (US)

(21) Appl. No.: 17/855,336

(22) Filed: Jun. 30, 2022

Related U.S. Application Data
(60) Provisional application No. 63/261,504, filed on Sep. 22, 2021.
(60) Provisional application No. 63/202,941, filed on Jun. 30, 2021.

(51) Int. Cl. C07D 471/22; C07D 405/14; C07D 495/12; C07D 401/04; C07D 519/00; C07D 491/048; C07D 471/14; C07D 471/04

(52) U.S. Cl. CPC C07D 471/22; C07D 405/14; C07D 495/12; C07D 401/04; C07D 519/00; C07D 491/048; C07D 471/14; C07D 471/04

(57) ABSTRACT

The present invention provides compounds, compositions thereof, and methods of using the same.

### CROSS-REFERENCE TO RELATED APPLICATIONS

This application claims the benefit of priority to U.S. Provisional Appl. Nos. 63/202,941, filed Jun. 30, 2021, and 63/261,504, filed Sep. 22, 2021, the entirety of each of which is herein incorporated by reference.

### TECHNICAL FIELD OF THE INVENTION

The present invention relates to compounds and methods useful for the modulation of mitogen-activated protein kinase-activated protein kinase-2 (MAPKAP kinase-2 or MK2) via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Berndsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth JS Jr., Chembiochem, 2005, 6(1):40-46).

An ongoing need exists in the art for effective treatments for disease, especially diseases and discorders mediated by pro-inflammatory molecules such as TNFα, IL-1, and IL-6. As such, small molecule therapeutic agents that leverage E3 ligase mediated protein degradation to pro-inflammatory associated proteins such as mitogen-activated protein kinase-activated protein kinase-2 (MAPKAP kinase-2 or MK2) hold promise as therapeutic agents. Accordingly, there remains a need to find compounds that are MK2 degraders useful as therapeutic agents.

### SUMMARY OF THE INVENTION

The present application relates novel bifunctional compounds, which function to recruit MK2 proteins to E3 ubiquitin ligase for degradation, and methods of preparation and uses thereof. In particular, the present disclosure provides bifunctional compounds, which find utility as modulators of targeted ubiquitination of MK2 proteins, which are then degraded and/or otherwise inhibited by the bifunctional compounds as described herein. Also provided are monovalent compounds, which find utility as inducers of targeted ubiquitination of MK2 proteins, which are then degraded and/or otherwise inhibited by the monovalent compounds as described herein. An advantage of the compounds provided herein is that a broad range of pharmacological activities is possible, consistent with the degradation/inhibition of MK2 proteins. In addition, the description provides methods of using an effective amount of the compounds as described herein for the treatment or amelioration of a disease condition, such as pain, inflammation, tissue damage, and arthritis.

The present application further relates to targeted degradation of MK2 proteins through the use of bifunctional molecules, including bifunctional molecules that link a cereblon-binding moiety to a ligand that binds MK2 proteins.

It has now been found that compounds of this invention, and pharmaceutically acceptable compositions thereof, are effective as degraders of MK2 proteins. Such compounds have the general formula I:

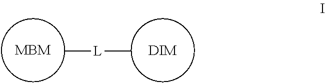

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of signaling pathways implicating MK2 proteins. Such diseases, disorders, or conditions include those described herein.

Compounds provided by this invention are also useful for the study of MK2 proteins in biological and pathological phenomena; the study of intracellular signal transduction pathways occurring in bodily tissues; and the comparative evaluation of new MK2 inhibitors or MK2 degraders or other regulators of cell cycling, metastasis, angiogenesis, and immune cell evasion, in vitro or in vivo.

### DETAILED DESCRIPTION OF CERTAIN EMBODIMENTS

### 1. General Description of Certain Embodiments of the Invention

Compounds of the present invention, and compositions thereof, are useful as degraders and/or inhibitors of one or more MK2 proteins.

In certain embodiments, the present invention provides a compound of formula I:

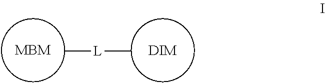

or a pharmaceutically acceptable salt thereof, wherein:
MBM is a MK2 binding moiety capable of binding to MK2;
L is a bivalent moiety that connects MBM to DIM; and
DIM is a degradation inducing moiety selected from an E3 ubiquitin ligase binding moeity (LBM), lysine mimetic, and hydrogen.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclic ring may be a 5-12 membered bicyclic, bridged bicyclic, or spirocyclic ring. A carbocyclic ring may include one or more oxo (═O) or thioxo (═S) substituent. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

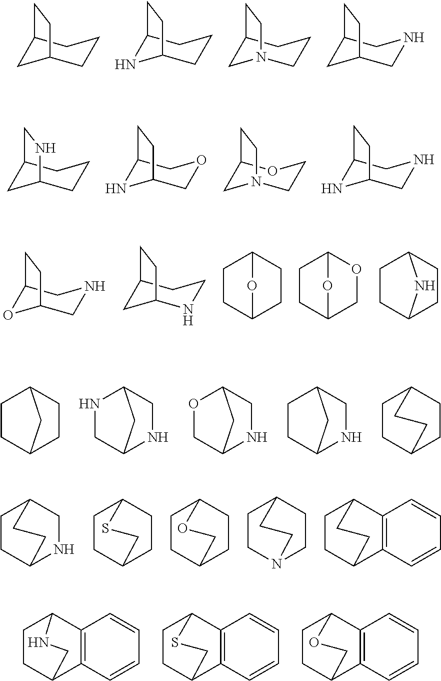

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

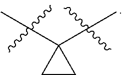 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. In some embodiments, a heterocyclic ring may be a 5-12 membered bicyclic, bridged bicyclic, or spirocyclic ring. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)_{0- 4}OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄₀S(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{•} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{•} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃₀—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromotagraphic purification are comtemplated herein (e.g., diammonium salts) and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

The term “prodrug” refers to a compound that is made more active in vivo. The present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

As used herein, the term “inhibitor” is defined as a compound that binds to and/or inhibits an MK2 protein with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and/or inhibits both an MK2 protein and an E3 ligase with measurable affinity resulting in the ubiquitination and subsequent degradation of the MK2 protein. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41:2596-99 and Sun et al., Bioconjugate Chem., 2006, 17:52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a MK2 protein activity between a sample comprising a compound of the present invention, or composition thereof, and a MK2 protein, and an equivalent sample comprising a MK2 protein, in the absence of said compound, or composition thereof.

### 3. Description of Exemplary Embodiments

As described above, in certain embodiments, the present invention provides a compound of formula I:

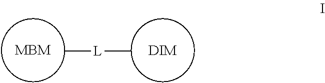

or a pharmaceutically acceptable salt thereof, wherein:
MBM is a MK2 protein binding moiety capable of binding to MK2;
L is a bivalent moiety that connects MBM to DIM; and
DIM is a degradation inducing moiety selected from an E3 ubiquitin ligase binding moeity (LBM), lysine mimetic, and hydrogen.

MK2 Binding Moiety (MBM)

In some embodiments, MBM is a MK2 protein binding moiety. Such MK2 binders are well known to one of ordinary skill in the art and include those described in WO 2004/017909, WO 2004/037814, WO 2004/054504, WO 2004/054505, US 2004/0127519, WO 2004/055015, WO 2004/055019, WO 2004/058176, WO 2004/058762, WO 2005/007092, WO 2005/009370, WO 2005/110410, WO 2007/038314, WO 2007/039285, WO 2007/117465, US 2008/045536, WO 2008/034600, WO 2008/154241, WO 2009/010488, WO 2009/011880, WO 2009/011871, WO 2009/012375, US 2009/175852, US 2009/203690, WO 2010/088368, WO 2011/017132, WO 2011/041784, WO 2011/073119, WO 2011/139295, US 2011/288036, WO 2012/016186, WO 2012/142458, WO 2013/033657, US 2013/058980, WO 2013/063095, US 2013/143906, WO 2013/134636, WO 2013/133876, JP 2014/009192, WO 2014/040074, US 2014/072613, JP 2014/088342, JP 2014/101340, WO 2014/149164, US 2014/314789, WO 2014/197846, WO 2015/006752, WO 2015/050957, WO 2015/138784, WO 2016/032882, US 2016/075720, WO 2016/049677, US 2016/158190, WO 2016/112292, WO 2016/135286, WO 2016/145234, US 2012/252737, US 2015/018279, US 2017/190713, WO 2018/170200, WO 2018/170204, WO 2018/170201, WO 2018/170203, WO 2018/170199, WO 2018/231722, US 2019/022176, CN 110724664, WO 2021/022186, and US 2021/154281, the entirety of each is herein incorporated by reference.

In certain embodiments, the present invention provides a compound of formula I, wherein MBM is a MK2 binding moiety thereby forming a compound of formula I-aa:

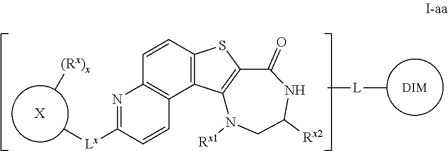

or a pharmaceutically acceptable salt thereof, wherein L and DIM are as defined above and described in embodiments herein, and wherein:
Ring X is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{x} is hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} is a covalent bond or a bivalent, saturated or partially unsaturated, straight or branched C₁₋₅ hydrocarbon chain, wherein 0-3 methylene units of I are independently replaced by —O—, —NR—, —CRF—, —CF₂—, —C(O)—, —S—, —S(O)—, or —S(O)₂—;
R^{x1} and R^{x2} are, independently, hydrogen or an optionally substituted C₁₋₆ aliphatic; and
x is 0, 1, 2, 3, or 4.

In certain embodiments, the present invention provides a compound of formula I, wherein MBM is a MK2 binding moiety thereby forming a compound of formula I′-aa:

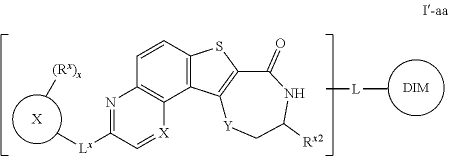

or a pharmaceutically acceptable salt thereof, wherein L and DIM are as defined above and described in embodiments herein, and wherein:
Ring X is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
X is an optionally substituted carbon or nitrogen atom;
Y is —O—, —S—, or —N(R^{x1})—;
R^{x} is hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} is a covalent bond or a bivalent, saturated or partially unsaturated, straight or branched C₁₋₅ hydrocarbon chain, wherein 0-3 methylene units of I are independently replaced by —O—, —NR—, —CRF—, —CF₂—, —C(O)—, —S—, —S(O)—, or —S(O)₂—;
R^{x1} and R^{x2} are, independently, hydrogen or an optionally substituted C₁₋₆ aliphatic; and
x is 0, 1, 2, 3, or 4.

In certain embodiments, the present invention provides a compound of formula I, wherein MBM is a MK2 binding moiety thereby forming a compound of formula I-bb-1 or I-bb-2:

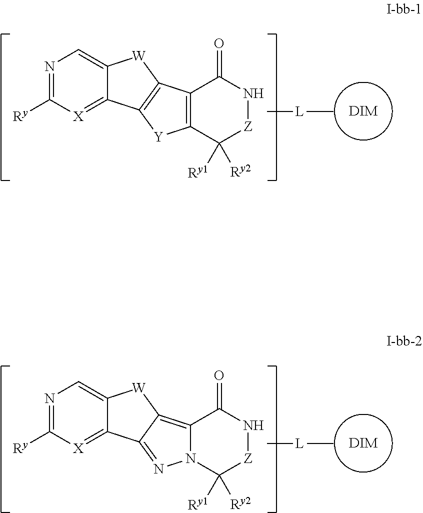

or a pharmaceutically acceptable salt thereof, wherein:
R^{y} is hydrogen, halogen, —Y¹—R, an optionally substituted C₁₋₆ aliphatic,

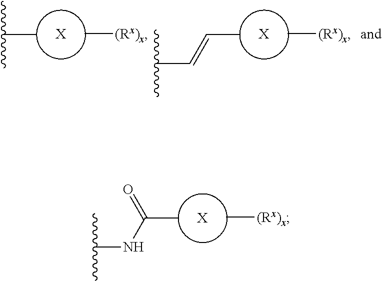

Ring X is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{x} is hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X is an optionally substituted carbon or nitrogen atom;
Y and Y¹ are, independently, a bivalent group selected from —O—, —S— and —NR—;
W is absent or a bivalent group selected from —CH₂—, —CH₂CH₂—, —CH₂CH₂CH₂— and —CH═CH—;
Z is a bivalent group selected from a bond and —C(R^{y3})(R^{y4})—;
R^{y1} and R^{y2} are, independently, hydrogen or taken together with their intervening atoms to form an optionally substituted 4-7 membered saturated or partially unsaturated carbocyclic or heterocyclic ring having 1-3 heteroatoms independently selected from nitrogen, oxygen, and sulfur;
R^{y3} and R^{y4} are, independently, hydrogen or taken together with their intervening atoms to form an optionally substituted 4-7 membered saturated or partially unsaturated carbocyclic or heterocyclic ring having 1-3 heteroatoms independently selected from nitrogen, oxygen, and sulfur; and
x is 0, 1, 2, 3, or 4.

In certain embodiments, the present invention provides a compound of formula I, wherein MBM is a MK2 binding moiety thereby forming a compound of formula I-cc:

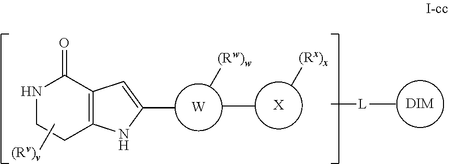

or a pharmaceutically acceptable salt thereof, wherein L and DIM are as defined above and described in embodiments herein, and wherein:
Ring W is a bivalent ring selected from phenylenyl, naphthylenyl, a 5-10 membered heteroarylenyl containing 1-4 heteroatoms independently selected from nitrogen, oxygen, and sulfur, and a 5-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur;
Ring X is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{v}, R^{w}, and R^{x} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
v is 0, 1, 2, 3, or 4;
w is 0, 1, 2, 3, or 4; and
x is 0, 1, 2, 3, or 4.

In certain embodiments, the present invention provides a compound of formula I, wherein MBM is a MK2 binding moiety thereby forming a compound of formula I′-cc:

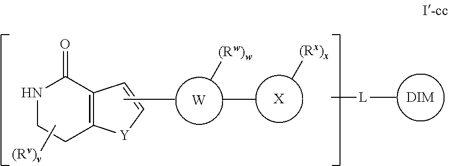

or a pharmaceutically acceptable salt thereof, wherein L and DIM are as defined above and described in embodiments herein, and wherein:
Y is —CH═CH—, —NH—, —O—, or —S—;
Ring W is a bivalent ring selected from phenylenyl, naphthylenyl, a 5-10 membered heteroarylenyl containing 1-4 heteroatoms independently selected from nitrogen, oxygen, and sulfur, and a 5-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur;
Ring X is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{v}, R^{w}, and R^{x} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R; or:
   two R^{v} groups on the same or different atoms are optionally taken together with their intervening atoms to form an optionally substituted 3-6 membered saturated or partially unsaturated carbocyclic ring or heterocyclic ring with 1-2 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-9 membered heteroaryl ring having 1-4 heteroatoms independently selected from nitrogen, oxygen, and sulfur;
v is 0, 1, 2, 3, or 4;
w is 0, 1, 2, 3, or 4; and
x is 0, 1, 2, 3, or 4.

In certain embodiments, the present invention provides a compound of formula I, wherein MBM is a MK2 binding moiety thereby forming a compound of formula I-dd:

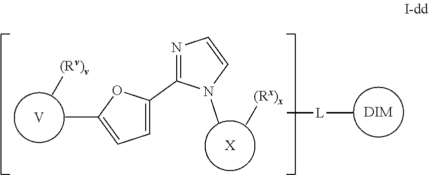

or a pharmaceutically acceptable salt thereof, wherein L and DIM are as defined above and described in embodiments herein, and wherein:
Ring V and Ring X are, independently, a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{v} and R^{x} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
v is 0, 1, 2, 3, or 4; and
x is 0, 1, 2, 3, or 4.

In certain embodiments, the present invention provides a compound of formula I, wherein MBM is a MK2 binding moiety thereby forming a compound of formula I-ee:

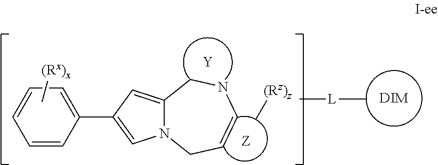

or a pharmaceutically acceptable salt thereof, wherein:
Ring Y is a fused 5-membered heterocyclic ring with 1-4 heteroatoms independently selected from nitrogen, oxygen, and sulfur;
Ring Z is benzo or a fused 5- to 6-membered heterocyclic ring with 1-4 heteroatoms independently selected from nitrogen, oxygen, and sulfur;
R^{x} and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
x is 0, 1, 2, 3, or 4; and
z is 0, 1, 2, 3, or 4.

In certain embodiments, the present invention provides a compound of formula I, wherein MBM is a MK2 binding moiety thereby forming a compound of formula I-ff:

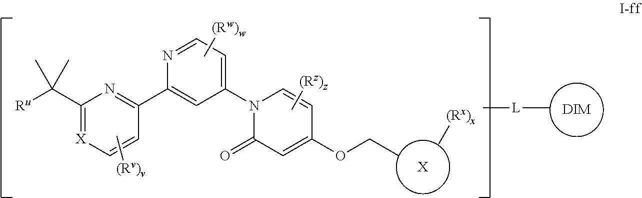

or a pharmaceutically acceptable salt thereof, wherein L and DIM are as defined above and described in embodiments herein, and wherein:
X is a carbon or nitrogen atom;
Ring X is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{u}, R^{v}, R^{w}, R^{x}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
v is 0, 1, 2, 3, or 4;
w is 0, 1, 2, 3, or 4;
x is 0, 1, 2, 3, or 4; and
z is 0, 1, 2, 3, or 4.

In certain embodiments, the present invention provides a compound of formula I, wherein MBM is a MK2 binding moiety thereby forming a compound of formula I′-ff:

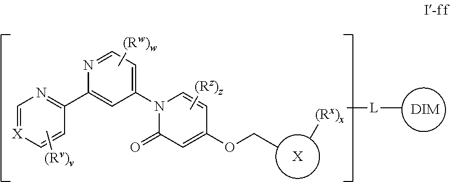

or a pharmaceutically acceptable salt thereof, wherein L and DIM are as defined above and described in embodiments herein, and wherein:
X is a carbon or nitrogen atom;
Ring X is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{v}, R^{w}, R^{x}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
v is 0, 1, 2, 3, or 4;
w is 0, 1, 2, 3, or 4;
x is 0, 1, 2, 3, or 4; and
z is 0, 1, 2, 3, or 4.

As defined above and described herein, Ring W is bivalent ring selected from phenylenyl, naphthylenyl, a 5-10 membered heteroarylenyl containing 1-4 heteroatoms independently selected from nitrogen, oxygen, and sulfur, and a 5-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur.

In some embodiments, Ring W is phenylenyl. In some embodiments, Ring W is naphthylenyl. In some embodiments, Ring W is a 5-10 membered heteroarylenyl containing 1-4 heteroatoms independently selected from nitrogen, oxygen, and sulfur. In some embodiments, Ring W is a 5-11 membered saturated or partially unsaturated monocyclic, bicyclic, bridged bicyclic, or spirocyclic carbocyclylenyl. In some embodiments, Ring W is a 5-11 membered saturated or partially unsaturated monocyclic, bicyclic, bridged bicyclic, or spirocyclic heterocyclylenyl with 1-4 heteroatoms independently selected from nitrogen, oxygen, and sulfur. In some embodiments, Ring X is pyrazolylenyl. In some embodiments, Ring W is pyridinylenyl. In some embodiments, Ring W is pyrimidinylenyl.

In some embodiments, Ring W is selected from those depicted in Table 1, below.

As defined above and described herein, Ring V and Ring X are selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur.

In some embodiments, Ring V is phenyl. In some embodiments, Ring V is naphthyl. In some embodiments, Ring V is a 5-10 membered heteroaryl containing 1-4 heteroatoms independently selected from nitrogen, oxygen, and sulfur. In some embodiments, Ring V is a 5-11 membered saturated or partially unsaturated monocyclic, bicyclic, bridged bicyclic, or spirocyclic carbocyclyl. In some embodiments, Ring V is a 5-11 membered saturated or partially unsaturated monocyclic, bicyclic, bridged bicyclic, or spirocyclic heterocyclyl with 1-4 heteroatoms independently selected from nitrogen, oxygen, and sulfur.

In some embodiments, Ring X is phenyl. In some embodiments, Ring X is naphthyl. In some embodiments, Ring X is a 5-10 membered heteroaryl containing 1-4 heteroatoms independently selected from nitrogen, oxygen, and sulfur. In some embodiments, Ring X is a 5-11 membered saturated or partially unsaturated monocyclic, bicyclic, bridged bicyclic, or spirocyclic carbocyclyl. In some embodiments, Ring X is a 5-11 membered saturated or partially unsaturated monocyclic, bicyclic, bridged bicyclic, or spirocyclic heterocyclyl with 1-4 heteroatoms independently selected from nitrogen, oxygen, and sulfur. In some embodiments, Ring X is quinolinyl. In some embodiments, Ring X is pyridinyl. In some embodiments, Ring X is piperzinyl. In some embodiments, Ring X is piperdinyl. In some embodiments, Ring X is pyrrolidinyl. In some embodiments, Ring X is azetinyl. In some embodiments, Ring X is

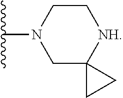 [SMILES: *N1CCNC2(CC2)C1]

In some embodiments, Ring X is

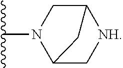 [SMILES: *N1CC2CC1CN2]

In some embodiments, Ring X is

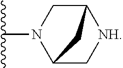 [SMILES: *N1C[C@H]2C[C@@H]1CN2]

In some embodiments, Ring X is

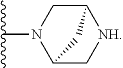 [SMILES: *N1C[C@@H]2C[C@H]1CN2]

In some embodiments, Ring X is

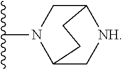 [SMILES: *N1CC2CCC1CN2]

In some embodiments, Ring X is

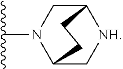 [SMILES: *N1C[C@H]2CC[C@@H]1CN2]

In some embodiments, Ring X is

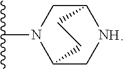

In some embodiments, Ring X is

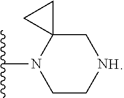

In some embodiments, Ring X is

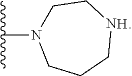

In some embodiments, Ring X is

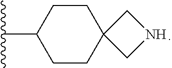 [SMILES: *C1CCC2(CC1)CNC2]

In some embodiments, Ring X is

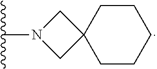 [SMILES: *N1CC2(CCCCC2)C1]

In some embodiments, Ring X is

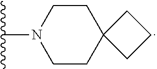 [SMILES: *N1CCC2(CCC2)CC1]

In some embodiments, Ring X is

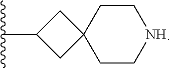 [SMILES: *C1CC2(CCNCC2)C1]

In some embodiments, Ring X is

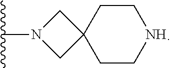 [SMILES: *N1CC2(CCNCC2)C1]

In some embodiments, Ring X is

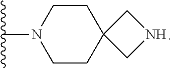 [SMILES: *N1CCC2(CC1)CNC2]

In some embodiments, Ring X is

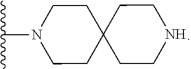 [SMILES: *N1CCC2(CCNCC2)CC1]

In some embodiments, Ring X and R^{w} are

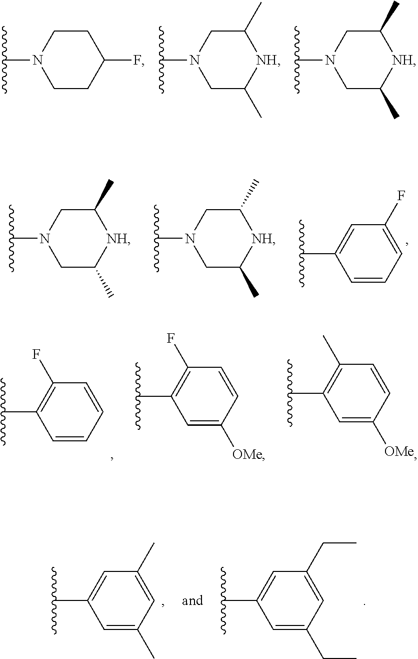

In some embodiments, Ring V and Ring X are selected from those depicted in Table 1, below.

As defined above and described herein, Ring Y is a fused 5-membered heterocyclic ring with 1-4 heteroatoms independently selected from nitrogen, oxygen, and sulfur.

In some embodiments, Ring Y is is a fused 5-membered heterocyclic ring with 1-4 heteroatoms independently selected from nitrogen, oxygen, and sulfur. In some embodiments, Ring Y is

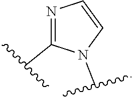 [SMILES: *c1nccn1*]

In some embodiments, Ring Y is selected from those depicted in Table 1, below.

As defined above and described herein, Ring Z is benzo or a fused 5- to 6-membered heterocyclic ring with 1-4 heteroatoms independently selected from nitrogen, oxygen, and sulfur.

In some embodiments, Ring Z is benzo. In some embodiments, Ring Z is a fused 5- to 6-membered heterocyclic ring with 1-4 heteroatoms independently selected from nitrogen, oxygen, and sulfur.

In some embodiments, Ring Z is selected from those depicted in Table 1, below.

As defined above and described herein, R^{u}, R^{v}, R^{w}, R^{x}, and R^{z}, are independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R.

As defined above and described herein, two R^{v} groups on the same or different atoms are optionally taken together with their intervening atoms to form an optionally substituted 3-6 membered saturated or partially unsaturated carbocyclic ring or heterocyclic ring with 1-2 heteroatoms independently selected from nitrogen, oxygen, and sulfur.

In some embodiments, one or more of R^{u}, R^{v}, R^{w}, R^{x}, and R^{z} are hydrogen. In some embodiments, one or more of R^{u}, R^{v}, R^{w}, R^{x}, and R^{z} are R^{A}. In some embodiments, one or more of R^{u}, R^{v}, R^{w}, R^{x}, and R^{z} are halogen. In some embodiments, one or more of R^{u}, R^{v}, R^{w}, R^{x}, and R^{z} are —CN. In some embodiments, one or more of R^{u}, R^{v}, R^{w}, R^{x}, and R^{z} are —NO₂. In some embodiments, one or more of R^{u}, R^{v}, R^{w}, R^{x}, and R^{z} are —OR. In some embodiments, one or more of R^{u}, R^{v}, R^{w}, R^{x}, and R^{z} are —SR. In some embodiments, one or more of R^{u}, R^{v}, R^{w}, R^{x}, and R^{z} are —N(R)₂. In some embodiments, one or more of R^{v}, R^{w}, R^{x}, and R^{z} are —Si(R)₃. In some embodiments, one or more of R^{u}, R^{v}, R^{w}, R^{x}, and R^{z} are —S(O)₂R. In some embodiments, one or more of R^{u}, R^{v}, R^{w}, R^{x}, and R^{z} are —S(O)₂N(R)₂. In some embodiments, one or more of R^{u}, R^{v}, R^{w}, R^{x}, and R^{z} are —S(O)R. In some embodiments, one or more of R^{u}, R^{v}, R^{w}, R^{x}, and R^{z} are —C(O)R. In some embodiments, one or more of R^{u}, R^{v}, R^{w}, R^{x}, and R^{z} are —C(O)OR. In some embodiments, one or more of R^{u}, R^{v}, R^{w}, R^{x}, and R^{z} are —C(O)N(R)₂. In some embodiments, one or more of R^{u}, R^{v}, R^{w}, R^{x}, and R^{z} are —C(O)NROR. In some embodiments, one or more of R^{u}, R^{v}, R^{w}, R^{x}, and R^{z} are —OC(O)R. In some embodiments, one or more of R^{u}, R^{v}, R^{w}, R^{x}, and R^{z} are —OC(O)N(R)₂. In some embodiments, one or more of R^{u}, R^{v}, R^{w}, R^{x}, and R^{z} are —OP(O)(R)₂. In some embodiments, one or more of R^{u}, R^{v}, R^{w}, R^{x}, and R^{z} are —OP(O)(OR)₂. In some embodiments, one or more of R^{u}, R^{v}, R^{w}, R^{x}, and R^{z} are —OP(O)(OR)N(R)₂. In some embodiments, one or more of R^{u}, R^{v}, R^{w}, R^{x}, and R^{z} are —OP(O)(N(R)₂)₂. In some embodiments, one or more of R^{u}, R^{v}, R^{w}, R^{x}, and R^{z} are —NRC(O)OR. In some embodiments, one or more of R^{u}, R^{v}, R^{w}, R^{x}, and R^{z} are —NRC(O)R. In some embodiments, one or more of R^{u}, R^{v}, R^{w}, R^{x}, and R^{z} are —NRC(O)N(R)₂. In some embodiments, one or more of R^{u}, R^{v}, R^{w}, R^{x}, and R^{z} are —NRS(O)₂R. In some embodiments, one or more of R^{u}, R^{v}, R^{w}, R^{x}, and R^{z} are —NP(O)(R)₂. In some embodiments, one or more of R^{u}, R^{v}, R^{w}, R^{x}, and R^{z} are —NRP(O)(OR)₂. In some embodiments, one or more of R^{u}, R^{v}, R^{w}, R^{x}, and R^{z} are —NRP(O)(OR)N(R)₂. In some embodiments, one or more of R^{u}, R^{v}, R^{w}, R^{x}, and R^{z} are —NRP(O)(N(R)₂)₂. In some embodiments, one or more of R^{u}, R^{v}, R^{w}, R^{x}, and R^{z} are —NRS(O)₂R.

In some embodiments, R^{u} is —OH.

In some embodiments, R^{v} is fluoro. In some embodiments, R^{v} is methyl. In some embodiments, R^{v} is —CN.

In some embodiments, R^{w} is fluoro. In some embodiments, R^{w} is chloro. In some embodiments, R^{w} is C₁₋₆alkyl. In some embodiments, R^{w} is methyl. In some embodiments, R^{w} is ethyl. In some embodiments, R^{w} is C₁₋₆haloalkyl. In some embodiments, R^{w} is —CF₃. In some embodiments, R^{w} is OC₁₋₆alkyl. In some embodiments, R^{w} is —OMe. In some embodiments, R^{w} is —NH₂.

In some embodiments, R^{x} is fluoro. In some embodiments, R^{x} is chloro. In some embodiments, R^{x} is C₁₋₆alkyl. In some embodiments, R^{x} is methyl. In some embodiments, R^{x} is ethyl. In some embodiments, R^{x} is isopropyl. In some embodiments, R^{x} is C₁₋₆cycloalkyl. In some embodiments, R^{x} is cyclopentyl. In some embodiments, R^{x} is phenyl. In some embodiments, R^{x} is indolyl. In some embodiments, R^{x} is C₁₋₆haloalkyl. In some embodiments, R^{x} is —CF₃. In some embodiments, R^{x} is —CN. In some embodiments, R^{x} is OC₁₋₆alkyl. In some embodiments, R^{x} is —OMe. In some embodiments, R^{x} is —OiPr. In some embodiments, R^{x} is —C(CH₃)₂OH. In some embodiments, R^{x} is

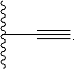 [SMILES: *C#C]

In some embodiments, R^{x} is

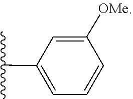 [SMILES: *c1cccc(OC)c1]

In some embodiments, R^{x} is

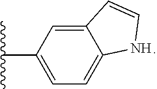 [SMILES: *c1ccc2[nH]ccc2c1]

In some embodiments, R^{z} is fluoro. In some embodiments, R^{z} is chloro. In some embodiments, R^{z} is methyl. In some embodiments, R^{z} is —CN. In some embodiments, R^{z} is —CF₃.

In some embodiments, two R^{v} groups on the same or different atoms are optionally taken together with their intervening atoms to form an optionally substituted 3-6 membered saturated or partially unsaturated carbocyclic ring. In some embodiments, two R^{v} groups on the same or different atoms are optionally taken together with their intervening atoms to form an optionally substituted 3-6 membered saturated or partially unsaturated heterocyclic ring with 1-2 heteroatoms independently selected from nitrogen, oxygen, and sulfur.

In some embodiments, two R groups form cyclopropylenyl. In some embodiments, two R^{v} groups form cyclobutylenyl. In some embodiments, two R^{v} groups form cyclopentylenyl.

In some embodiments, R^{u}, R^{v}, R^{w}, R^{x}, and R^{z}, are selected from those depicted in Table 1, below.

As defined above and described herein, each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur.

In some embodiments, R is hydrogen. In some embodiments, R is optionally substituted C₁₋₆ aliphatic. In some embodiments, R is optionally substituted phenyl. In some embodiments, R is optionally substituted 3-7 membered saturated or partially unsaturated heterocyclic ring having 1-2 heteroatoms independently selected from nitrogen, oxygen, and sulfur. In some embodiments, R is optionally substituted 5-6 membered heteroaryl ring having 1-4 heteroatoms independently selected from nitrogen, oxygen, and sulfur. In some embodiments, two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur. In some embodiments, where R^{z} is —N(R)₂, two R groups form a 7- to 9-membered spirocyclic heterocyclic ring, such as

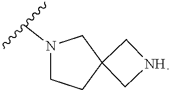 [SMILES: *N1CCC2(CNC2)C1]

In some embodiments, Ring R is selected from those depicted in Table 1, below.

As defined above and described herein, each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-9 membered heteroaryl ring having 1-4 heteroatoms independently selected from nitrogen, oxygen, and sulfur.

In some embodiments, R^{A} is an optionally substituted C₁₋₆ aliphatic. In some embodiments, R^{A} is C₁₋₆alkyl (e.g., methyl, ethyl, isopropyl, etc.). In some embodiments, R^{A} is C₁₋₆haloalkyl (e.g., —CF₃, —CHF₂, etc.). In some embodiments, R^{A} is an optionally substituted phenyl. In some embodiments, R^{A} is an optionally substituted 3-7 membered saturated or partially unsaturated carbocyclic ring. In some embodiments, R^{A} is an optionally substituted 3-7 membered saturated or partially unsaturated heterocyclic ring having 1-2 heteroatoms selected from nitrogen, oxygen, and sulfur. In some embodiments, R^{A} is an optionally substituted 5-9 membered heteroaryl ring having 1-4 heteroatoms independently selected from nitrogen, oxygen, and sulfur.

In some embodiments, R^{A} is selected from those depicted in Table 1, below.

As defined above and described herein, L^{x} is a covalent bond or a bivalent, saturated or partially unsaturated, straight or branched C₁₋₅ hydrocarbon chain, wherein 0-3 methylene units of L are independently replaced by —O—, —NR—, —CRF—, —CF₂—, —C(O)—, —S—, —S(O)—, or —S(O)₂—.

In some embodiments, L^{x} is a covalent bond. In some embodiments, L^{x} is a bivalent, saturated or partially unsaturated, straight or branched C₁₋₅ hydrocarbon chain, wherein 0-3 methylene units of L^{x} are independently replaced by —O—, —NR—, —CRF—, —CF₂—, —C(O)—, —S—, —S(O)—, or —S(O)₂—.

In some embodiments, L^{x} is selected from those depicted in Table 1, below.

As defined above and described herein, R^{x1} and R^{x2} are, independently, hydrogen or an optionally substituted C₁₋₆ aliphatic.

In some embodiments, R is hydrogen. In some embodiments, R^{x1} is an optionally substituted C₁₋₆ aliphatic. In some embodiments, R^{x2} is hydrogen. In some embodiments, R^{x2} is an optionally substituted C₁₋₆ aliphatic. In some embodiments, R^{x2} is methyl.

In some embodiments, R^{x1} and R^{x2} are selected from those depicted in Table 1, below.

As defined above and described herein, x is 0, 1, 2, 3, or 4.

In some embodiments, x is 0. In some embodiments, x is 1. In some embodiments, x is 2. In some embodiments, x is 3. In some embodiments, x is 4.

In some embodiments, x is selected from those depicted in Table 1, below.

As defined above and described herein, R^{y} is hydrogen, halogen, —Y¹—R, an optionally substituted C₁₋₆ aliphatic,

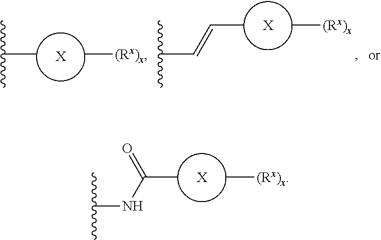

In some embodiments, R^{y} is hydrogen. In some embodiments, R^{y} is halogen. In some embodiments, R^{y} is —Y¹—R. In some embodiments, R^{y} is an optionally substituted C₁₋₆ aliphatic. In some embodiments, R^{y} is

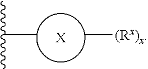

In some embodiments, R^{y} is

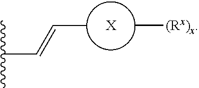

In some embodiments, R^{y} is

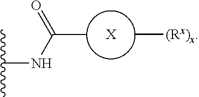

In some embodiments, R^{y} is phenyl. In some embodiments, R^{y} is

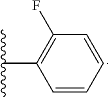 [SMILES: *c1ccccc1F]

In some embodiments, Ring R^{y} is selected from those depicted in Table 1, below.

As defined above and described herein, X is an optionally substituted carbon or nitrogen atom.

In some embodiments, X is carbon atom. In some embodiments, X is nitrogen atom. In some embodiments, X is a carbon atom substituted with fluoro.

In some embodiments, X is selected from those depicted in Table 1, below.

As defined above and described herein, Y and Y¹ are, independently, a bivalent group selected from —CH═CH—, —O—, —S—, —NR—, and —N(R^{x1})—.

In some embodiments, Y is —CH═CH—. In some embodiments, Y is —O—. In some embodiments, Y is —S—. In some embodiments, Y is —NR—. In some embodiments, Y is —N(R^{x1})—. In some embodiments, Y¹ is —O—. In some embodiments, Y¹ is —S—. In some embodiments, Y¹ is —NR—.

In some embodiments, Y and Y¹ are selected from those depicted in Table 1, below.

As defined above and described herein, W is absent or a bivalent group selected from —CH₂—, —CH₂CH₂—, —CH₂CH₂CH₂— and —CH═CH—.

In some embodiments, W is absent. In some embodiments, W is —CH₂—. In some embodiments, W is —CH₂CH₂—. In some embodiments, W is —CH₂CH₂CH₂—. In some embodiments, W is —CH═CH—.

In some embodiments, W is selected from those depicted in Table 1, below.

As defined above and described herein, Z is a bivalent group selected from a bond and —C(R^{y3})(R^{y4})—.

In some embodiments, Z is a bond. In some embodiments, Z is —C(R^{y3})(R^{y4})—.

In some embodiments, Z is selected from those depicted in Table 1, below.

As defined above and described herein, R^{y1} and R^{y2} are, independently, hydrogen or taken together with their intervening atoms to form an optionally substituted 4-7 membered saturated or partially unsaturated carbocyclic or heterocyclic ring having 1-3 heteroatoms independently selected from nitrogen, oxygen, and sulfur.

In some embodiments, R^{y1} is hydrogen. In some embodiments, R^{y2} is hydrogen. In some embodiments, R^{y1} and R^{y2} are taken together with their intervening atoms to form an optionally substituted 4-7 membered saturated or partially unsaturated carbocyclic ring. In some embodiments, R^{y1} and R^{y2} are taken together with their intervening atoms to form an optionally substituted 4-7 membered saturated or partially unsaturated heterocyclic ring having 1-3 heteroatoms independently selected from nitrogen, oxygen, and sulfur. In some embodiments, R^{y1} and R^{y2} are taken together to form

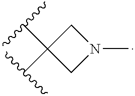 [SMILES: *C1(*)CN(C)C1]

In some embodiments, R^{y1} and R^{y2} are taken together to form

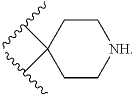 [SMILES: *C1(*)CCNCC1]

In some embodiments, R^{y1} and R^{y2} are selected from those depicted in Table 1, below.

As defined above and described herein, R^{y3} and R^{y4} are, independently, hydrogen or taken together with their intervening atoms to form an optionally substituted 4-7 membered saturated or partially unsaturated carbocyclic or heterocyclic ring having 1-3 heteroatoms independently selected from nitrogen, oxygen, and sulfur.

In some embodiments, R^{y3} is hydrogen. In some embodiments, R^{y4} is hydrogen. In some embodiments, R^{y3} and R^{y4} are taken together with their intervening atoms to form an optionally substituted 4-7 membered saturated or partially unsaturated carbocyclic ring. In some embodiments, R^{y3} and R^{y4} are taken together with their intervening atoms to form an optionally substituted 4-7 membered saturated or partially unsaturated heterocyclic ring having 1-3 heteroatoms independently selected from nitrogen, oxygen, and sulfur.

In some embodiments, R^{y3} and R^{y4} are selected from those depicted in Table 1, below.

In some embodiments, MBM is

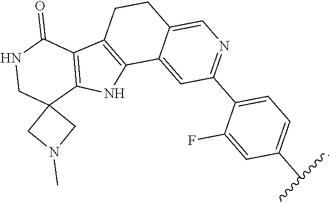 [SMILES: *c1ccc(-c2cc3c(cn2)CCc2c-3[nH]c3c2C(=O)NCC32CN(C)C2)c(F)c1]

In some embodiments, MBM is

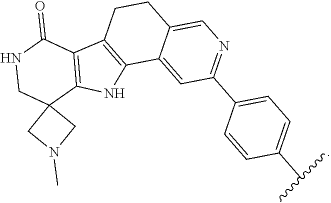 [SMILES: *c1ccc(-c2cc3c(cn2)CCc2c-3[nH]c3c2C(=O)NCC32CN(C)C2)cc1]

In some embodiments, MBM is

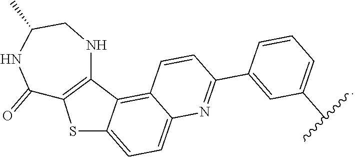 [SMILES: *c1cccc(-c2ccc3c(ccc4sc5c(c43)NC[C@@H](C)NC5=O)n2)c1]

In some embodiments, MBM is

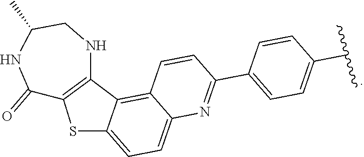 [SMILES: *c1ccc(-c2ccc3c(ccc4sc5c(c43)NC[C@@H](C)NC5=O)n2)cc1]

In some embodiments, MBM is

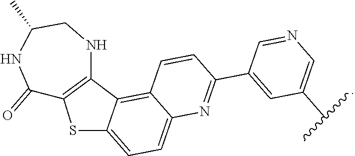 [SMILES: *c1cncc(-c2ccc3c(ccc4sc5c(c43)NC[C@@H](C)NC5=O)n2)c1]

In some embodiments, MBM is

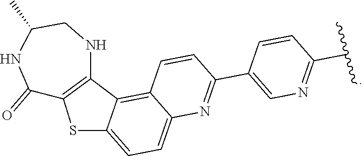 [SMILES: *c1ccc(-c2ccc3c(ccc4sc5c(c43)NC[C@@H](C)NC5=O)n2)cn1]

In some embodiments, MBM is

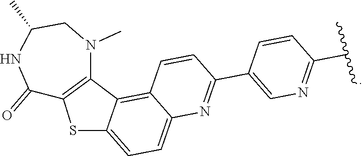 [SMILES: *c1ccc(-c2ccc3c(ccc4sc5c(c43)N(C)C[C@@H](C)NC5=O)n2)cn1]

In some embodiments, MBM is

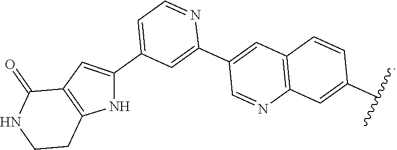 [SMILES: *c1ccc2cc(-c3cc(-c4cc5c([nH]4)CCNC5=O)ccn3)cnc2c1]

In some embodiments, MBM is

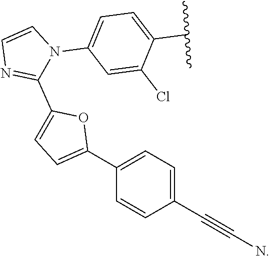

In some embodiments, MBM is

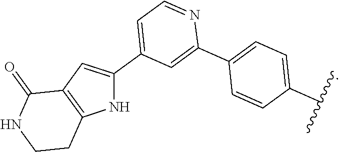 [SMILES: *c1ccc(-c2cc(-c3cc4c([nH]3)CCNC4=O)ccn2)cc1]

In some embodiments, MBM is

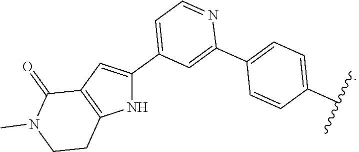 [SMILES: *c1ccc(-c2cc(-c3cc4c([nH]3)CCN(C)C4=O)ccn2)cc1]

In some embodiments, MBM is

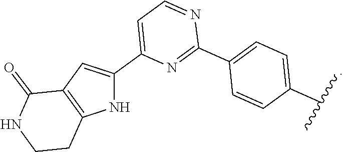 [SMILES: *c1ccc(-c2nccc(-c3cc4c([nH]3)CCNC4=O)n2)cc1]

In some embodiments, MBM is

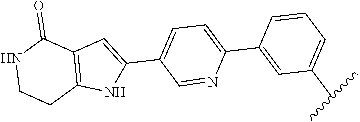 [SMILES: *c1cccc(-c2ccc(-c3cc4c([nH]3)CCNC4=O)cn2)c1]

In some embodiments, MBM is

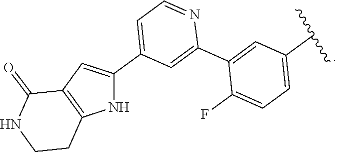 [SMILES: *c1ccc(F)c(-c2cc(-c3cc4c([nH]3)CCNC4=O)ccn2)c1]

In some embodiments, MBM is

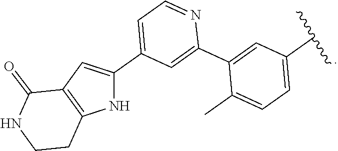 [SMILES: *c1ccc(C)c(-c2cc(-c3cc4c([nH]3)CCNC4=O)ccn2)c1]

In some embodiments, MBM is

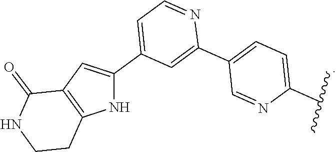 [SMILES: *c1ccc(-c2cc(-c3cc4c([nH]3)CCNC4=O)ccn2)cn1]

In some embodiments, MBM is

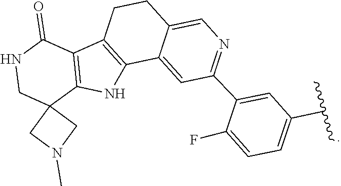 [SMILES: *c1ccc(F)c(-c2cc3c(cn2)CCc2c-3[nH]c3c2C(=O)NCC32CN(C)C2)c1]

In some embodiments, MBM is

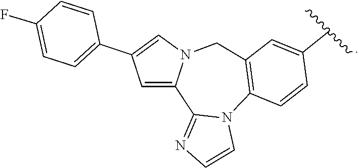 [SMILES: *c1ccc2c(c1)Cn1cc(-c3ccc(F)cc3)cc1-c1nccn1-2]

In some embodiments, MBM is

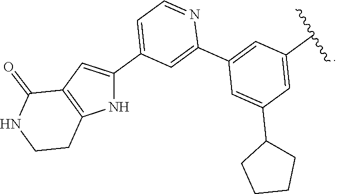 [SMILES: *c1cc(-c2cc(-c3cc4c([nH]3)CCNC4=O)ccn2)cc(C2CCCC2)c1]

In some embodiments, MBM is

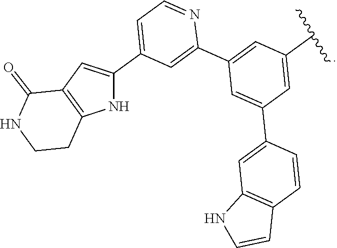 [SMILES: *c1cc(-c2ccc3cc[nH]c3c2)cc(-c2cc(-c3cc4c([nH]3)CCNC4=O)ccn2)c1]

In some embodiments, MBM is

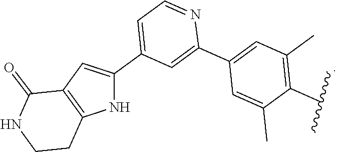 [SMILES: *c1c(C)cc(-c2cc(-c3cc4c([nH]3)CCNC4=O)ccn2)cc1C]

In some embodiments, MBM is

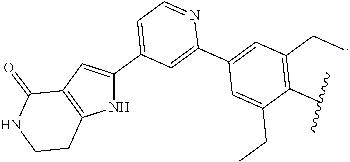 [SMILES: *c1c(CC)cc(-c2cc(-c3cc4c([nH]3)CCNC4=O)ccn2)cc1CC]

In some embodiments, MBM is

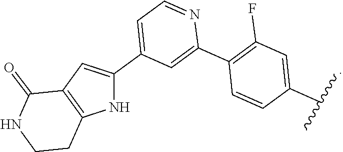 [SMILES: *c1ccc(-c2cc(-c3cc4c([nH]3)CCNC4=O)ccn2)c(F)c1]

In some embodiments, MBM is

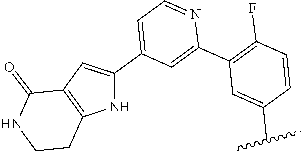 [SMILES: *c1ccc(F)c(-c2cc(-c3cc4c([nH]3)CCNC4=O)ccn2)c1]

In some embodiments, MBM is

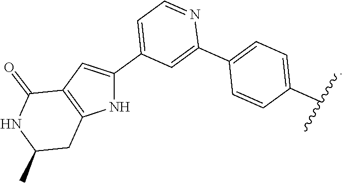 [SMILES: *c1ccc(-c2cc(-c3cc4c([nH]3)C[C@@H](C)NC4=O)ccn2)cc1]

In some embodiments, MBM is

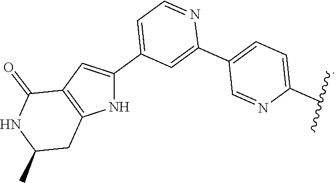 [SMILES: *c1ccc(-c2cc(-c3cc4c([nH]3)C[C@@H](C)NC4=O)ccn2)cn1]

In some embodiments, MBM is

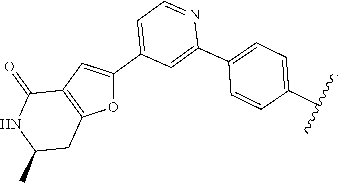 [SMILES: *c1ccc(-c2cc(-c3cc4c(o3)C[C@@H](C)NC4=O)ccn2)cc1]

In some embodiments, MBM is

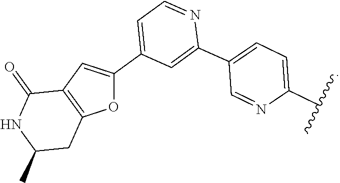 [SMILES: *c1ccc(-c2cc(-c3cc4c(o3)C[C@@H](C)NC4=O)ccn2)cn1]

In some embodiments, MBM is

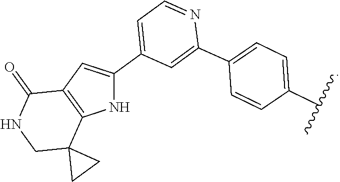 [SMILES: *c1ccc(-c2cc(-c3cc4c([nH]3)C3(CC3)CNC4=O)ccn2)cc1]

In some embodiments, MBM is

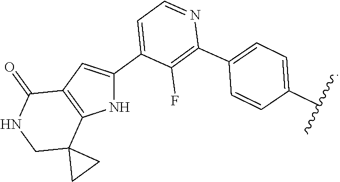 [SMILES: *c1ccc(-c2nccc(-c3cc4c([nH]3)C3(CC3)CNC4=O)c2F)cc1]

In some embodiments, MBM is

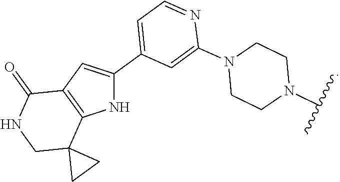 [SMILES: *N1CCN(c2cc(-c3cc4c([nH]3)C3(CC3)CNC4=O)ccn2)CC1]

In some embodiments, MBM is

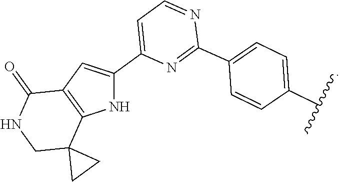 [SMILES: *c1ccc(-c2nccc(-c3cc4c([nH]3)C3(CC3)CNC4=O)n2)cc1]

In some embodiments, MBM is

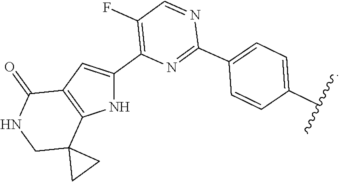 [SMILES: *c1ccc(-c2ncc(F)c(-c3cc4c([nH]3)C3(CC3)CNC4=O)n2)cc1]

In some embodiments, MBM is

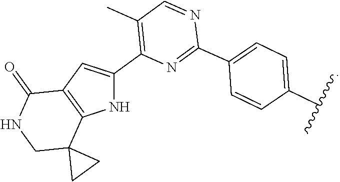 [SMILES: *c1ccc(-c2ncc(C)c(-c3cc4c([nH]3)C3(CC3)CNC4=O)n2)cc1]

In some embodiments, MBM is

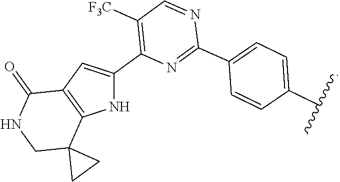 [SMILES: *c1ccc(-c2ncc(C(F)(F)F)c(-c3cc4c([nH]3)C3(CC3)CNC4=O)n2)cc1]

In some embodiments, MBM is

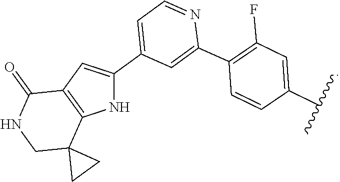 [SMILES: *c1ccc(-c2cc(-c3cc4c([nH]3)C3(CC3)CNC4=O)ccn2)c(F)c1]

In some embodiments, MBM is

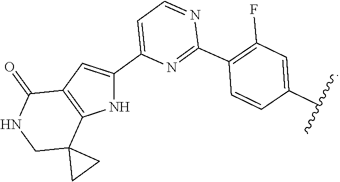 [SMILES: *c1ccc(-c2nccc(-c3cc4c([nH]3)C3(CC3)CNC4=O)n2)c(F)c1]

In some embodiments, MBM is

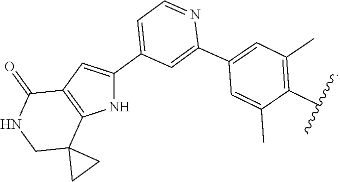 [SMILES: *c1c(C)cc(-c2cc(-c3cc4c([nH]3)C3(CC3)CNC4=O)ccn2)cc1C]

In some embodiments, MBM is

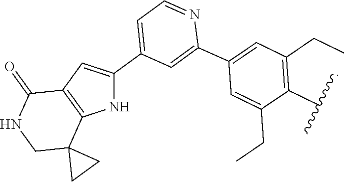 [SMILES: *c1c(CC)cc(-c2cc(-c3cc4c([nH]3)C3(CC3)CNC4=O)ccn2)cc1CC]

In some embodiments, MBM is

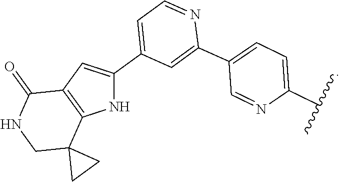 [SMILES: *c1ccc(-c2cc(-c3cc4c([nH]3)C3(CC3)CNC4=O)ccn2)cn1]

In some embodiments, MBM is

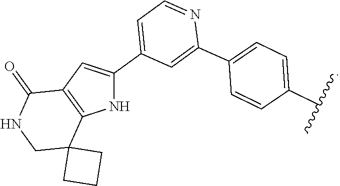 [SMILES: *c1ccc(-c2cc(-c3cc4c([nH]3)C3(CCC3)CNC4=O)ccn2)cc1]

In some embodiments, MBM is

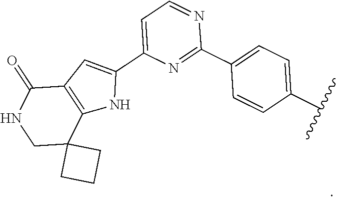 [SMILES: *c1ccc(-c2nccc(-c3cc4c([nH]3)C3(CCC3)CNC4=O)n2)cc1]

In some embodiments, MBM is

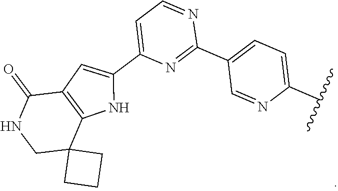 [SMILES: *c1ccc(-c2nccc(-c3cc4c([nH]3)C3(CCC3)CNC4=O)n2)cn1]

In some embodiments, MBM is

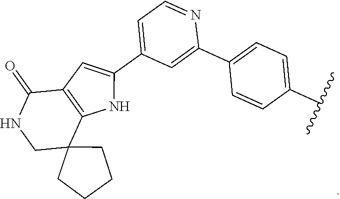 [SMILES: *c1ccc(-c2cc(-c3cc4c([nH]3)C3(CCCC3)CNC4=O)ccn2)cc1]

In some embodiments, MBM is

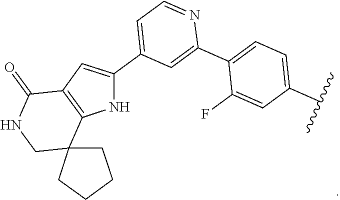 [SMILES: *c1ccc(-c2cc(-c3cc4c([nH]3)C3(CCCC3)CNC4=O)ccn2)c(F)c1]

In some embodiments, MBM is

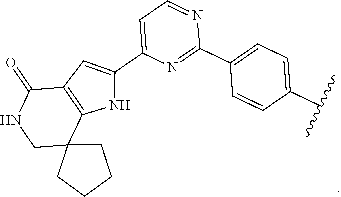 [SMILES: *c1ccc(-c2nccc(-c3cc4c([nH]3)C3(CCCC3)CNC4=O)n2)cc1]

In some embodiments, MBM is

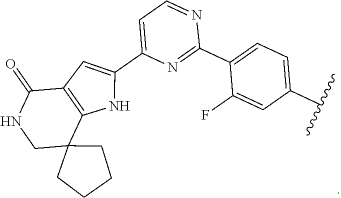 [SMILES: *c1ccc(-c2nccc(-c3cc4c([nH]3)C3(CCCC3)CNC4=O)n2)c(F)c1]

In some embodiments, MBM is

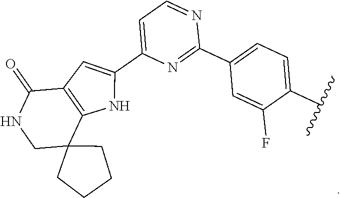 [SMILES: *c1ccc(-c2nccc(-c3cc4c([nH]3)C3(CCCC3)CNC4=O)n2)cc1F]

In some embodiments, MBM is

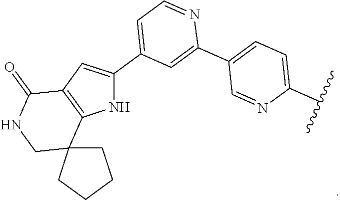 [SMILES: *c1ccc(-c2cc(-c3cc4c([nH]3)C3(CCCC3)CNC4=O)ccn2)cn1]

In some embodiments, MBM is

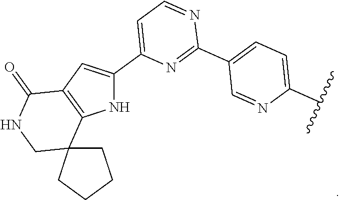 [SMILES: *c1ccc(-c2nccc(-c3cc4c([nH]3)C3(CCCC3)CNC4=O)n2)cn1]

In some embodiments, MBM is

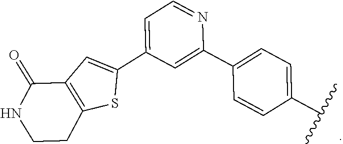 [SMILES: *c1ccc(-c2cc(-c3cc4c(s3)CCNC4=O)ccn2)cc1]

In some embodiments, MBM is

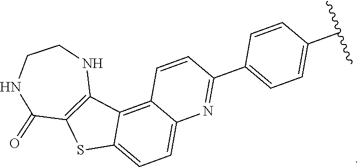 [SMILES: *c1ccc(-c2ccc3c(ccc4sc5c(c43)NCCNC5=O)n2)cc1]

In some embodiments, MBM is

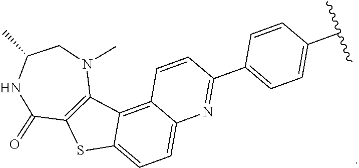 [SMILES: *c1ccc(-c2ccc3c(ccc4sc5c(c43)N(C)C[C@@H](C)NC5=O)n2)cc1]

In some embodiments, MBM is

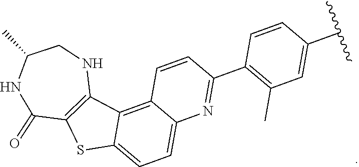 [SMILES: *c1ccc(-c2ccc3c(ccc4sc5c(c43)NC[C@@H](C)NC5=O)n2)c(C)c1]

In some embodiments, MBM is

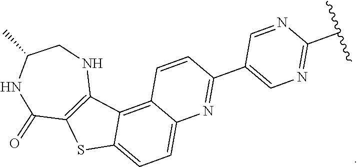 [SMILES: *c1ncc(-c2ccc3c(ccc4sc5c(c43)NC[C@@H](C)NC5=O)n2)cn1]

In some embodiments, MBM is

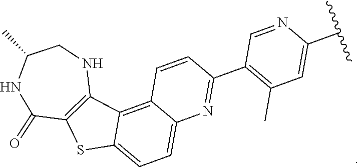 [SMILES: *c1cc(C)c(-c2ccc3c(ccc4sc5c(c43)NC[C@@H](C)NC5=O)n2)cn1]

In some embodiments, MBM is

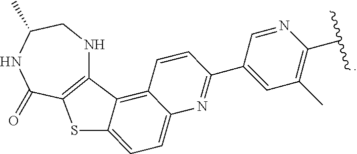 [SMILES: *c1ncc(-c2ccc3c(ccc4sc5c(c43)NC[C@@H](C)NC5=O)n2)cc1C]

In some embodiments, MBM is

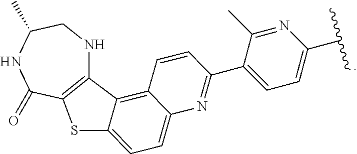 [SMILES: *c1ccc(-c2ccc3c(ccc4sc5c(c43)NC[C@@H](C)NC5=O)n2)c(C)n1]

In some embodiments, MBM is

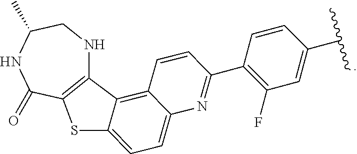 [SMILES: *c1ccc(-c2ccc3c(ccc4sc5c(c43)NC[C@@H](C)NC5=O)n2)c(F)c1]

In some embodiments, MBM is

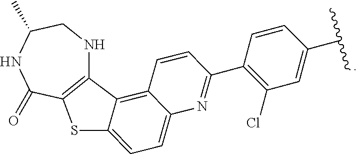 [SMILES: *c1ccc(-c2ccc3c(ccc4sc5c(c43)NC[C@@H](C)NC5=O)n2)c(Cl)c1]

In some embodiments, MBM is

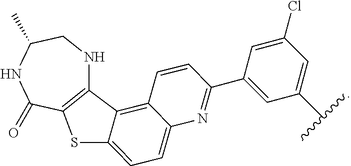 [SMILES: *c1cc(Cl)cc(-c2ccc3c(ccc4sc5c(c43)NC[C@@H](C)NC5=O)n2)c1]

In some embodiments, MBM is

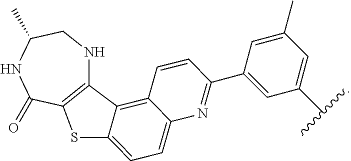 [SMILES: *c1cc(C)cc(-c2ccc3c(ccc4sc5c(c43)NC[C@@H](C)NC5=O)n2)c1]

In some embodiments, MBM is

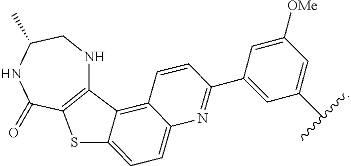 [SMILES: *c1cc(OC)cc(-c2ccc3c(ccc4sc5c(c43)NC[C@@H](C)NC5=O)n2)c1]

In some embodiments, MBM is

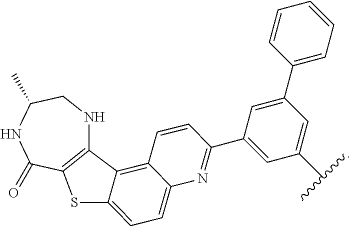 [SMILES: *c1cc(-c2ccccc2)cc(-c2ccc3c(ccc4sc5c(c43)NC[C@@H](C)NC5=O)n2)c1]

In some embodiments, MBM is

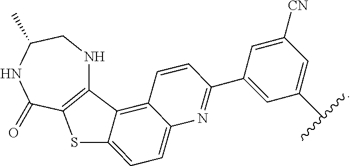 [SMILES: *c1cc(C#N)cc(-c2ccc3c(ccc4sc5c(c43)NC[C@@H](C)NC5=O)n2)c1]

In some embodiments, MBM is

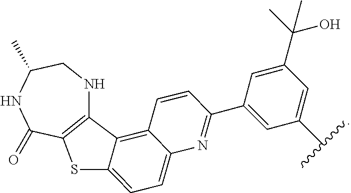 [SMILES: *c1cc(-c2ccc3c(ccc4sc5c(c43)NC[C@@H](C)NC5=O)n2)cc(C(C)(C)O)c1]

In some embodiments, MBM is

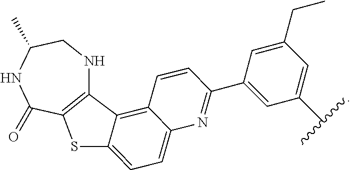 [SMILES: *c1cc(CC)cc(-c2ccc3c(ccc4sc5c(c43)NC[C@@H](C)NC5=O)n2)c1]

In some embodiments, MBM is

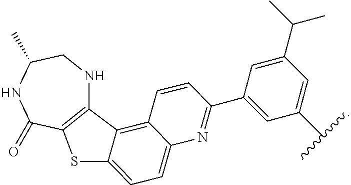 [SMILES: *c1cc(-c2ccc3c(ccc4sc5c(c43)NC[C@@H](C)NC5=O)n2)cc(C(C)C)c1]

In some embodiments, MBM is

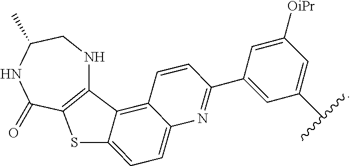 [SMILES: *c1cc(OC(C)C)cc(-c2ccc3c(ccc4sc5c(c43)NC[C@@H](C)NC5=O)n2)c1]

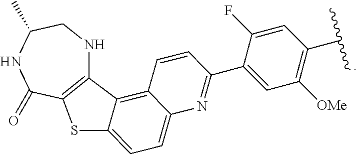 [SMILES: *c1cc(F)c(-c2ccc3c(ccc4sc5c(c43)NC[C@@H](C)NC5=O)n2)cc1OC]

In some embodiments, MBM is

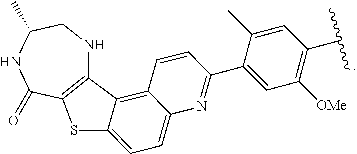 [SMILES: *c1cc(C)c(-c2ccc3c(ccc4sc5c(c43)NC[C@@H](C)NC5=O)n2)cc1OC]

In some embodiments, MBM is

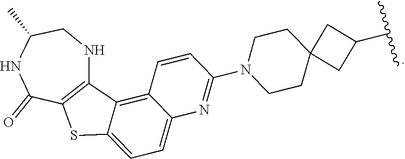 [SMILES: *C1CC2(CCN(c3ccc4c(ccc5sc6c(c54)NC[C@@H](C)NC6=O)n3)CC2)C1]

In some embodiments, MBM is

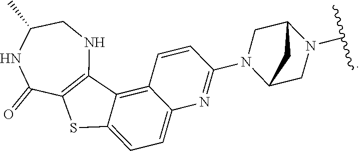 [SMILES: *N1C[C@H]2C[C@@H]1CN2c1ccc2c(ccc3sc4c(c32)NC[C@@H](C)NC4=O)n1]

In some embodiments, MBM is

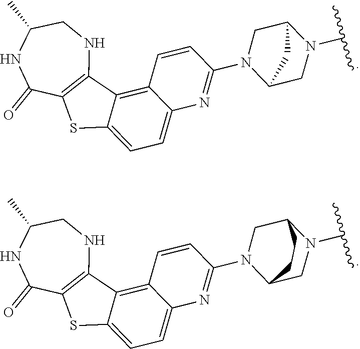 [SMILES: *N1C[C@@H]2C[C@H]1CN2c1ccc2c(ccc3sc4c(c32)NC[C@@H](C)NC4=O)n1 ; *N1C[C@H]2CC[C@@H]1CN2c1ccc2c(ccc3sc4c(c32)NC[C@@H](C)NC4=O)n1]

In some embodiments, MBM is

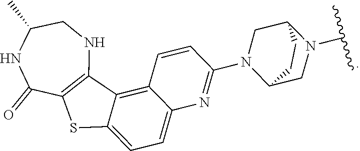 [SMILES: *N1C[C@@H]2CC[C@H]1CN2c1ccc2c(ccc3sc4c(c32)NC[C@@H](C)NC4=O)n1]

In some embodiments, MBM is

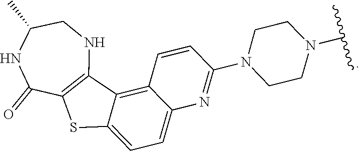 [SMILES: *N1CCN(c2ccc3c(ccc4sc5c(c43)NC[C@@H](C)NC5=O)n2)CC1]

In some embodiments, MBM is

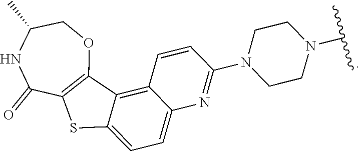 [SMILES: *N1CCN(c2ccc3c(ccc4sc5c(c43)OC[C@@H](C)NC5=O)n2)CC1]

In some embodiments, MBM is

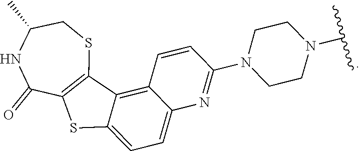 [SMILES: *N1CCN(c2ccc3c(ccc4sc5c(c43)SC[C@@H](C)NC5=O)n2)CC1]

In some embodiments, MBM is

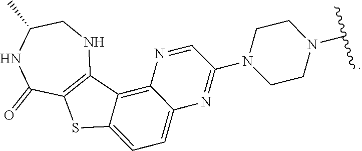 [SMILES: *N1CCN(c2cnc3c(ccc4sc5c(c43)NC[C@@H](C)NC5=O)n2)CC1]

In some embodiments, MBM is

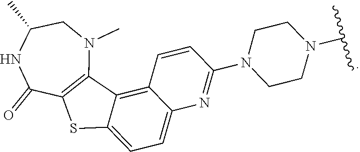 [SMILES: *N1CCN(c2ccc3c(ccc4sc5c(c43)N(C)C[C@@H](C)NC5=O)n2)CC1]

In some embodiments, MBM is

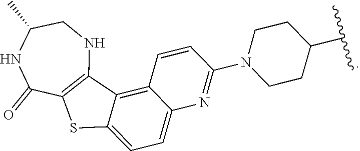 [SMILES: *C1CCN(c2ccc3c(ccc4sc5c(c43)NC[C@@H](C)NC5=O)n2)CC1]

In some embodiments, MBM is

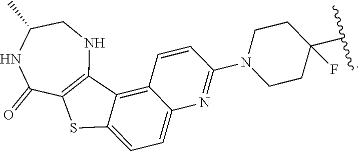 [SMILES: *C1(F)CCN(c2ccc3c(ccc4sc5c(c43)NC[C@@H](C)NC5=O)n2)CC1]

In some embodiments, MBM is

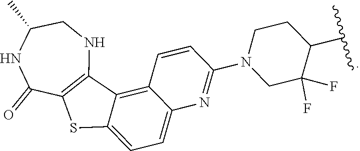 [SMILES: *C1CCN(c2ccc3c(ccc4sc5c(c43)NC[C@@H](C)NC5=O)n2)CC1(F)F]

In some embodiments, MBM is

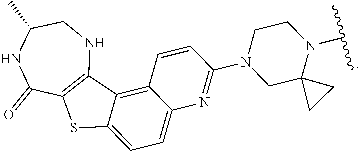 [SMILES: *N1CCN(c2ccc3c(ccc4sc5c(c43)NC[C@@H](C)NC5=O)n2)CC12CC2]

In some embodiments, MBM is

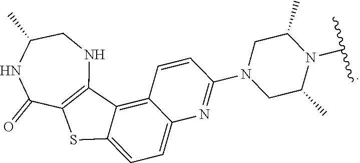 [SMILES: *N1[C@H](C)CN(c2ccc3c(ccc4sc5c(c43)NC[C@@H](C)NC5=O)n2)C[C@@H]1C]

In some embodiments, MBM is

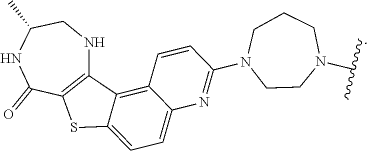 [SMILES: *N1CCCN(c2ccc3c(ccc4sc5c(c43)NC[C@@H](C)NC5=O)n2)CC1]

In some embodiments, MBM is

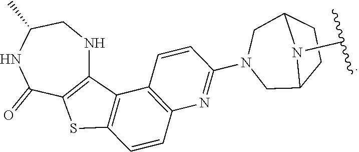 [SMILES: *N1C2CCC1CN(c1ccc3c(ccc4sc5c(c43)NC[C@@H](C)NC5=O)n1)C2]

In some embodiments, MBM is

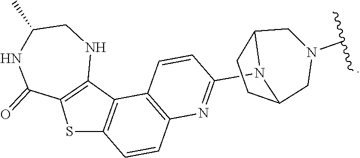 [SMILES: *N1CC2CCC(C1)N2c1ccc2c(ccc3sc4c(c32)NC[C@@H](C)NC4=O)n1]

In some embodiments, MBM is

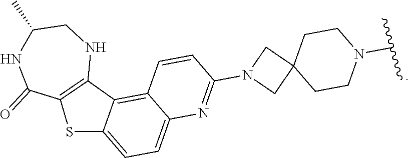 [SMILES: *N1CCC2(CC1)CN(c1ccc3c(ccc4sc5c(c43)NC[C@@H](C)NC5=O)n1)C2]

In some embodiments, MBM is

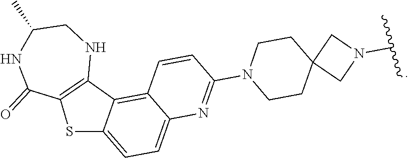 [SMILES: *N1CC2(CCN(c3ccc4c(ccc5sc6c(c54)NC[C@@H](C)NC6=O)n3)CC2)C1]

In some embodiments, MBM is

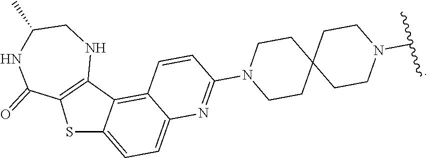 [SMILES: *N1CCC2(CC1)CCN(c1ccc3c(ccc4sc5c(c43)NC[C@@H](C)NC5=O)n1)CC2]

In some embodiments, MBM is

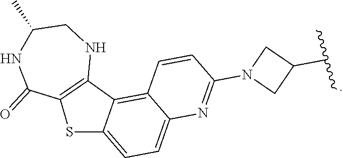 [SMILES: *C1CN(c2ccc3c(ccc4sc5c(c43)NC[C@@H](C)NC5=O)n2)C1]

In some embodiments, MBM is

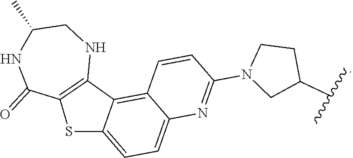 [SMILES: *C1CCN(c2ccc3c(ccc4sc5c(c43)NC[C@@H](C)NC5=O)n2)C1]

In some embodiments, MBM is

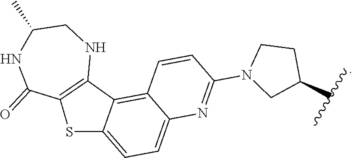 [SMILES: *[C@@H]1CCN(c2ccc3c(ccc4sc5c(c43)NC[C@@H](C)NC5=O)n2)C1]

In some embodiments, MBM is

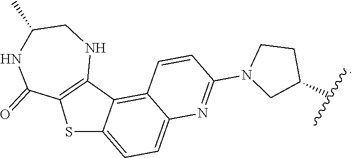 [SMILES: *[C@H]1CCN(c2ccc3c(ccc4sc5c(c43)NC[C@@H](C)NC5=O)n2)C1]

In some embodiments, MBM is

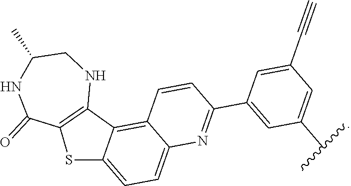 [SMILES: *c1cc(C#C)cc(-c2ccc3c(ccc4sc5c(c43)NC[C@@H](C)NC5=O)n2)c1]

In some embodiments, MBM is

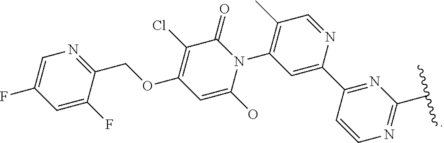 [SMILES: *c1nccc(-c2cc(-n3c(O)cc(OCc4ncc(F)cc4F)c(Cl)c3=O)c(C)cn2)n1]

In some embodiments, MBM is

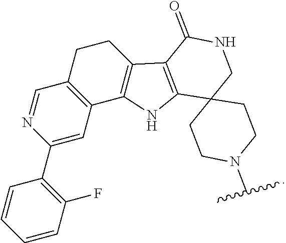 [SMILES: *N1CCC2(CC1)CNC(=O)c1c2[nH]c2c1CCc1cnc(-c3ccccc3F)cc1-2]

In some embodiments, MBM is

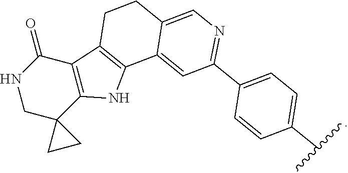 [SMILES: *c1ccc(-c2cc3c(cn2)CCc2c-3[nH]c3c2C(=O)NCC32CC2)cc1]

In some embodiments, MBM is

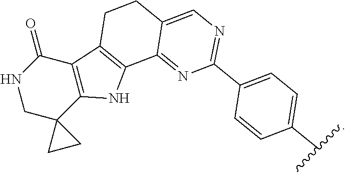 [SMILES: *c1ccc(-c2ncc3c(n2)-c2[nH]c4c(c2CC3)C(=O)NCC42CC2)cc1]

In some embodiments, MBM is

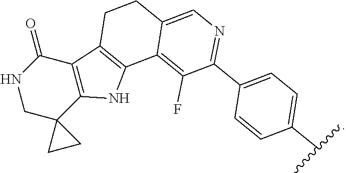 [SMILES: *c1ccc(-c2ncc3c(c2F)-c2[nH]c4c(c2CC3)C(=O)NCC42CC2)cc1]

In some embodiments, MBM is selected from those depicted in Table 1, below.

In some embodiments, the present invention provides a compound of formula I, wherein MBM is a MK2 binding moiety thereby forming a compound of formula I-gg-1:

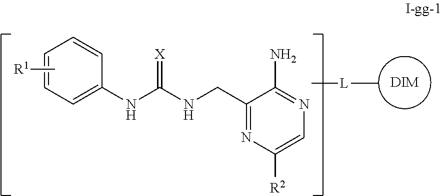

or a pharmaceutically acceptable salt thereof, wherein L and DIM are as defined above and described in embodiments herein, and wherein each of the variables R¹ and R² is as described in Anderson et al., Bioorg. Med. Chem. Lett. 2005, 15:1587-1590, the entirety of which is herein incorporated by reference.

In some embodiments, the present invention provides a compound of formula I, wherein MBM is a MK2 binding moiety thereby forming a compound of formula I-gg-2:

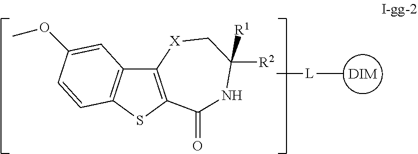

or a pharmaceutically acceptable salt thereof, wherein L and DIM are as defined above and described in embodiments herein, and wherein each of the variables X, R¹, and R² is as described in Anderson et al., Bioorg. Med. Chem. Lett. 2009, 19:4878-4881, the entirety of which is herein incorporated by reference.

In some embodiments, the present invention provides a compound of formula I, wherein MBM is a MK2 binding moiety thereby forming a compound of formula I-gg-3:

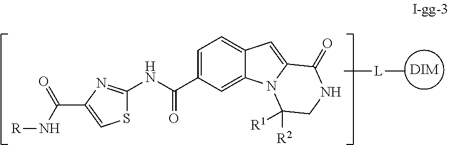

or a pharmaceutically acceptable salt thereof, wherein L and DIM are as defined above and described in embodiments herein, and wherein each of the variables R, R¹, and R² is as described in Goldbert et al., Bioorg. Med. Chem. Lett. 2008, 18:938-941, the entirety of which is herein incorporated by reference.

In some embodiments, the present invention provides a compound of formula I, wherein MBM is a MK2 binding moiety thereby forming a compound of formulae I-gg-4 or I-gg-5:

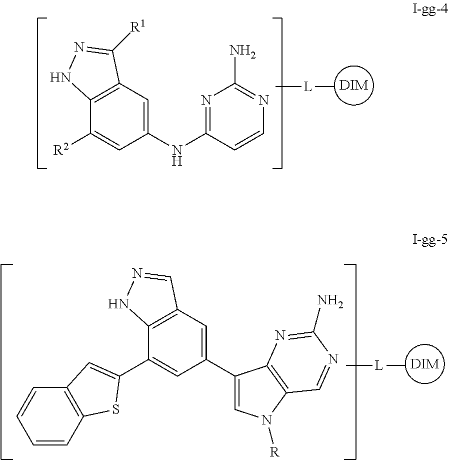

or a pharmaceutically acceptable salt thereof, wherein L and DIM are as defined above and described in embodiments herein, and wherein each of the variables R, R¹, and R² is as described in Harris et al., Bioorg. Med. Chem. Lett. 2010, 20:334-337, the entirety of which is herein incorporated by reference.

In some embodiments, the present invention provides a compound of formula I-gg-4, or a pharmaceutically acceptable salt thereof, wherein L and DIM are as defined above and described in embodiments herein, and wherein:

R¹ is hydrogen, halogen, thiophenyl, naphthyl, benzothiophenyl, or pyrrolyl;
R² is hydrogen, halogen, thiophenyl, naphthyl, benzothiophenyl, or benzofuranyl.

In some embodiments, the present invention provides a compound of formula I, wherein MBM is a MK2 binding moiety thereby forming a compound of formula I-gg-6:

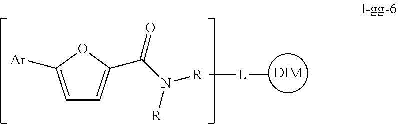

or a pharmaceutically acceptable salt thereof, wherein L and DIM are as defined above and described in embodiments herein, and wherein each of the variables Ar and R is as described in Huang et al., ACS Med. Chem. Lett. 2011, 2:632-637, the entirety of which is herein incorporated by reference.

In some embodiments, the present invention provides a compound of formula I, wherein MBM is a MK2 binding moiety thereby forming a compound of formulae I-gg-7 or I-gg-8:

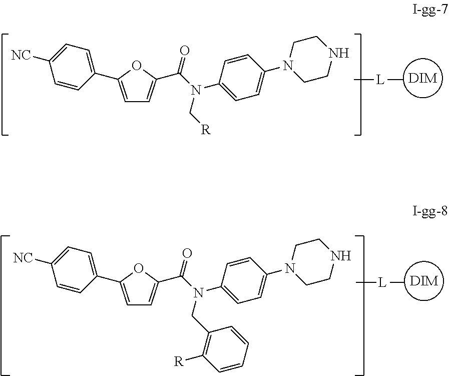

or a pharmaceutically acceptable salt thereof, wherein L and DIM are as defined above and described in embodiments herein, and wherein each of the variables R is as described in Huang et al., Bioorg. Med. Chem. Lett. 2012, 22:65-70, the entirety of which is herein incorporated by reference.

In some embodiments, the present invention provides a compound of formula I, wherein MBM is a MK2 binding moiety thereby forming a compound of formula I-gg-9:

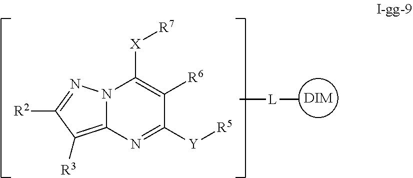

or a pharmaceutically acceptable salt thereof, wherein L and DIM are as defined above and described in embodiments herein, and wherein each of the variables X, Y, R², R³, R⁵, R⁶, and R⁷ is as described in Kosugi et al., J. Med. Chem. 2012, 55:6700-6715, the entirety of which is herein incorporated by reference.

In some embodiments, the present invention provides a compound of formula I, wherein MBM is a MK2 binding moiety thereby forming a compound of formula I-gg-10:

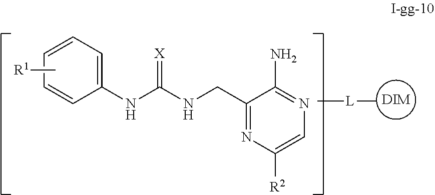

or a pharmaceutically acceptable salt thereof, wherein L and DIM are as defined above and described in embodiments herein, and wherein each of the variables X, R¹ and R² is as described in Lin et al., Bioorg. Med. Chem. Lett. 2009, 19:3238-3242, the entirety of which is herein incorporated by reference.

In some embodiments, the present invention provides a compound of formula I, wherein MBM is a MK2 binding moiety thereby forming a compound of formulae I-gg-11 or I-gg-12:

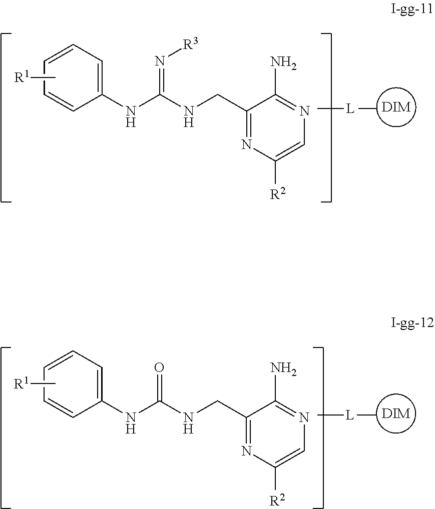

or a pharmaceutically acceptable salt thereof, wherein L and DIM are as defined above and described in embodiments herein, and wherein each of the variables R¹, R², and R³ is as described in Lin et al., Bioorg. Med. Chem. Lett. 2015, 25(22):5402-5408, the entirety of which is herein incorporated by reference.

In some embodiments, the present invention provides a compound of formula I, wherein MBM is a MK2 binding moiety thereby forming a compound of formula I-gg-13:

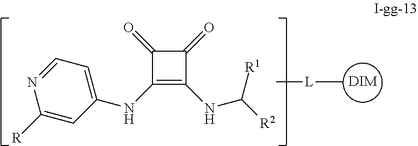

or a pharmaceutically acceptable salt thereof, wherein L and DIM are as defined above and described in embodiments herein, and wherein each of the variables R, R¹ and R² is as described in Lovering et al., Bioorg. Med. Chem. Lett. 2009, 17:3342-3351, the entirety of which is herein incorporated by reference.

In some embodiments, the present invention provides a compound of formula I, wherein MBM is a MK2 binding moiety thereby forming a compound of formula I-gg-14:

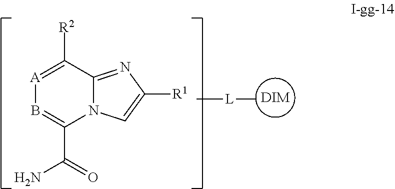

or a pharmaceutically acceptable salt thereof, wherein L and DIM are as defined above and described in embodiments herein, and wherein each of the variables A, B, R¹ and R² is as described in Meng et al., Bioorg. Med. Chem. Lett. 2013, 23:2863-2867, the entirety of which is herein incorporated by reference.

In some embodiments, the present invention provides a compound of formula I, wherein MBM is a MK2 binding moiety thereby forming a compound of formula I-gg-15:

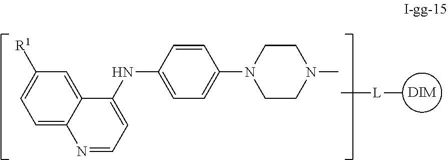

or a pharmaceutically acceptable salt thereof, wherein L and DIM are as defined above and described in embodiments herein, and wherein each of the variable R¹ is as described in Olsson et al., Bioorg. Med. Chem. Lett. 2010, 20:4738-4740, the entirety of which is herein incorporated by reference.

In some embodiments, the present invention provides a compound of formula I, wherein MBM is a MK2 binding moiety thereby forming a compound of formula I-gg-16:

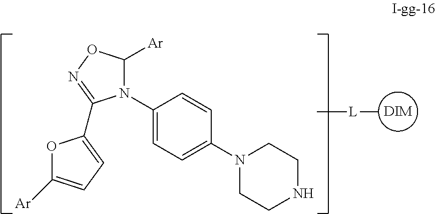

or a pharmaceutically acceptable salt thereof, wherein L and DIM are as defined above and described in embodiments herein, and wherein each of the variables Ar is as described in Qin et al., ACS Med. Chem. Lett. 2013, 3:100-105, the entirety of which is herein incorporated by reference.

In some embodiments, the present invention provides a compound of formula I, wherein MBM is a MK2 binding moiety thereby forming a compound of formula I-gg-17 or I-gg-18:

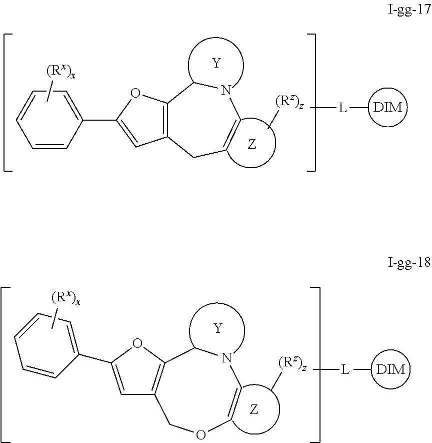

or a pharmaceutically acceptable salt thereof, wherein L, DIM, Ring Y, Ring Z, R^{x}, R^{z}, x, and z are as defined above and described in embodiments herein, both singly and in combination.

In some embodiments, the present invention provides a compound of formula I, wherein MBM is a MK2 binding moiety thereby forming a compound of formula I-gg-19:

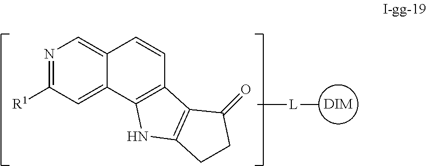

or a pharmaceutically acceptable salt thereof, wherein L and DIM are as defined above and described in embodiments herein, and wherein each of the variables R¹ is as described in Revesz et al., Bioorg. Med. Chem. Lett. 2010, 20:4715-4718, the entirety of which is herein incorporated by reference.

In some embodiments, the present invention provides a compound of formula I, wherein MBM is a MK2 binding moiety thereby forming a compound of formula I-gg-20:

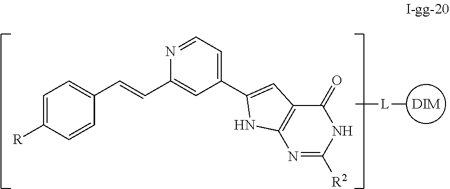

or a pharmaceutically acceptable salt thereof, wherein L and DIM are as defined above and described in embodiments herein, and wherein each of the variables R and R² is as described in Schlapback et al., Bioorg. Med. Chem. Lett. 2008, 18:6142-6146, the entirety of which is herein incorporated by reference.

In some embodiments, the present invention provides a compound of formula I, wherein MBM is a MK2 binding moiety thereby forming a compound of formula I-gg-21:

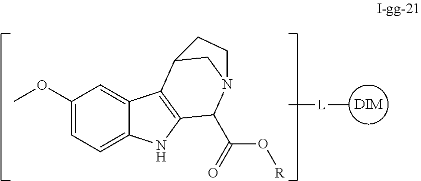

or a pharmaceutically acceptable salt thereof, wherein L and DIM are as defined above and described in embodiments herein, and wherein each of the variable R is as described in Trujillo et al., Bioorg. Med. Chem. Lett. 2007, 17:4657-4663, the entirety of which is herein incorporated by reference.

In some embodiments, the present invention provides a compound of formula I, wherein MBM is a MK2 binding moiety thereby forming a compound of formula I-gg-22:

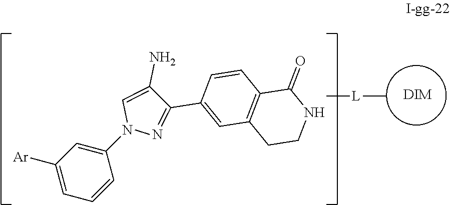

or a pharmaceutically acceptable salt thereof, wherein L and DIM are as defined above and described in embodiments herein, and wherein variables Ar is as described in Velcicky et al., Bioorg. Med. Chem. Lett. 2010, 20:1293-1297, the entirety of which is herein incorporated by reference.

In some embodiments, the present invention provides a compound of formula I, wherein MBM is a MK2 binding moiety thereby forming a compound of formula I-gg-23:

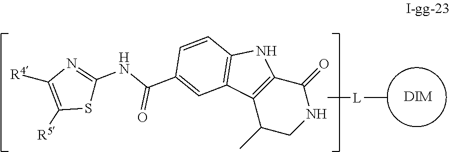

or a pharmaceutically acceptable salt thereof, wherein L and DIM are as defined above and described in embodiments herein, and wherein each of the variables R^{4′} and R^{5′} is as described in Wu et al., Bioorg. Med. Chem. Lett. 2007, 17:4664-4669, the entirety of which is herein incorporated by reference.

In some embodiments, the present invention provides a compound of formula I, wherein MBM is a MK2 binding moiety thereby forming a compound of formula I-gg-24:

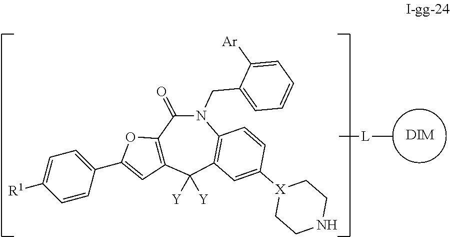

or a pharmaceutically acceptable salt thereof, wherein L and DIM are as defined above and described in embodiments herein, and wherein each of the variables R¹, Ar, X, and Y is as described in Xiao et al., Bioorg. Med. Chem. Lett. 2013, 24:3262-3266, the entirety of which is herein incorporated by reference.

In some embodiments, the present invention provides a compound of formula I, wherein MBM is a MK2 binding moiety thereby forming a compound of formula I-gg-25:

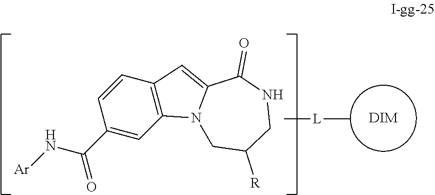

or a pharmaceutically acceptable salt thereof, wherein L and DIM are as defined above and described in embodiments herein, and wherein each of the variables Ar and R is as described in Xiong et al., Bioorg. Med. Chem. Lett. 2007, 18:1994-1999, the entirety of which is herein incorporated by reference.

### Ligase Binding Moiety (LBM)

In some embodiments, LBM is an E3 ligase ligand. Such E3 ligase ligands are well known to one of ordinary skill in the art and include those described in M. Toure, C. M. Crews, Angew. Chem. Int. Ed. 2016, 55, 1966, T. Uehara et al. Nature Chemical Biology 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, WO 2017/197046, WO 2017/197051, WO 2017/197055, and WO 2017/197056 each of, the entirety of each of which is herein incorporated by reference.

As defined herein and described below, wherein a formula is depicted using square brackets, e.g,

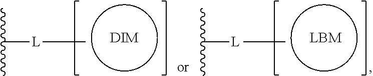

L is attached to a modifiable carbon, oxygen, or nitrogen atom within DIM or LBM including substitution or replacement of a defined group in DIM or LBM.

In certain embodiments, the present invention provides a compound of formula I, wherein LBM is an IMiD-based (immunomodulatory imide drug-based) cereblon E3 ubiquitin ligase binding moiety thereby forming a compound of formula I-a-1, I-a-2, I-a-3, I-a-4, I-a-5, I-a-6, I-a-7, I-a-8, I-a-9, or I-a-10 respectively:

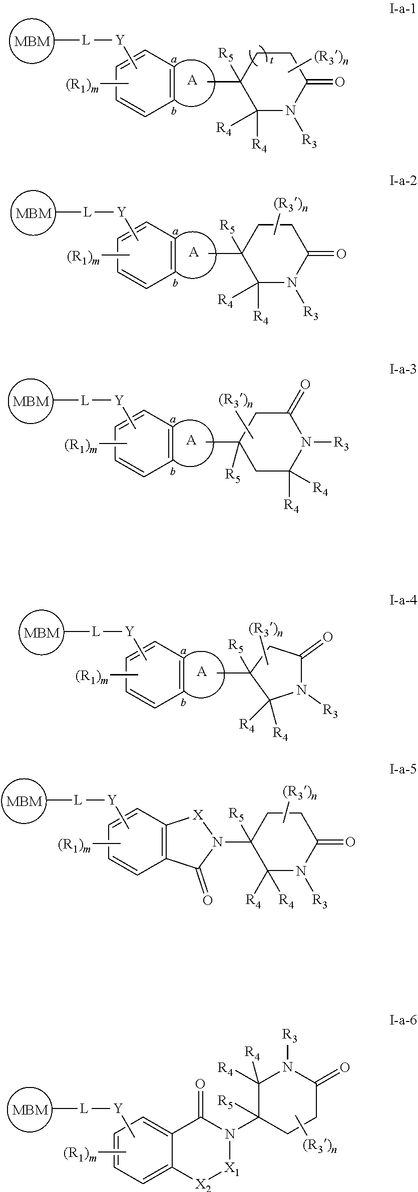

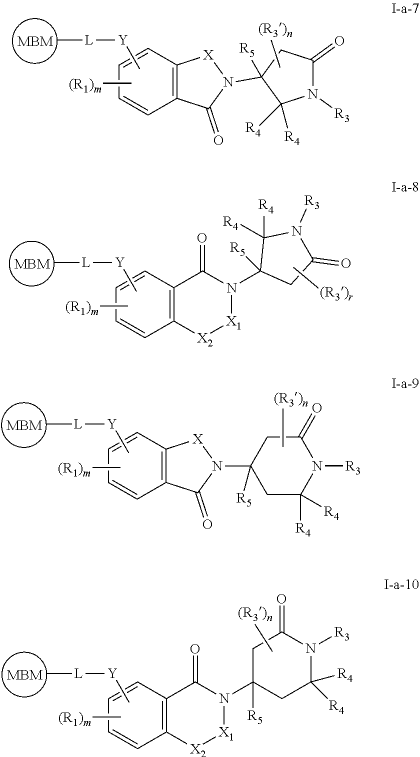

or a compound of formula I-a′-1, I-a′-2, I-a′-3, I-a′-4, I-a′-5, I-a′-6, I-a′-7, I-a′-8, I-a′-9, or I-a′-10 respectively:

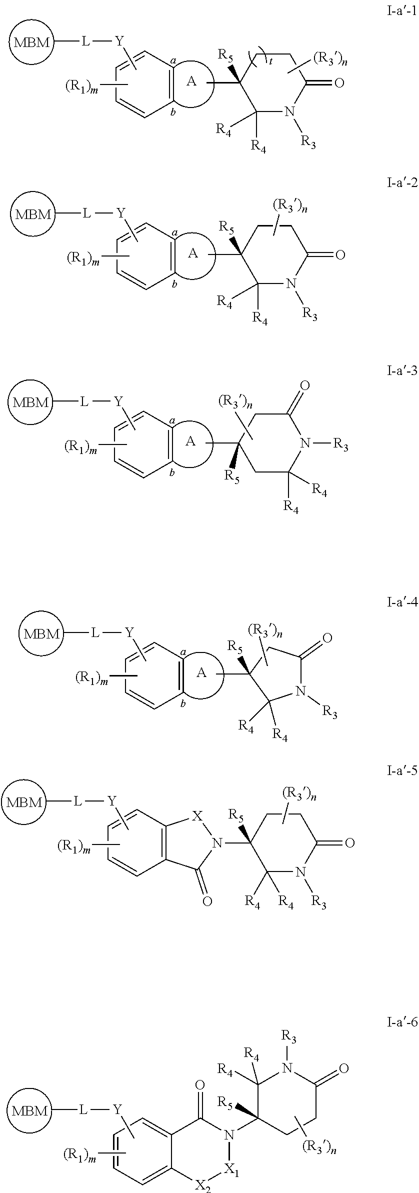

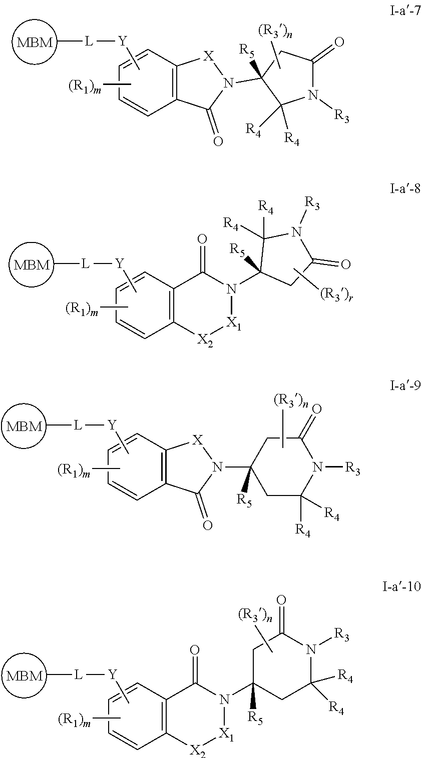

or a compound of formula I-a″-1, I-a″-2, I-a″-3, I-a″-4, I-a″-5, I-a″-6, I-a″-7, I-a″-8, I-a″-9, or I-a″-10 respectively:

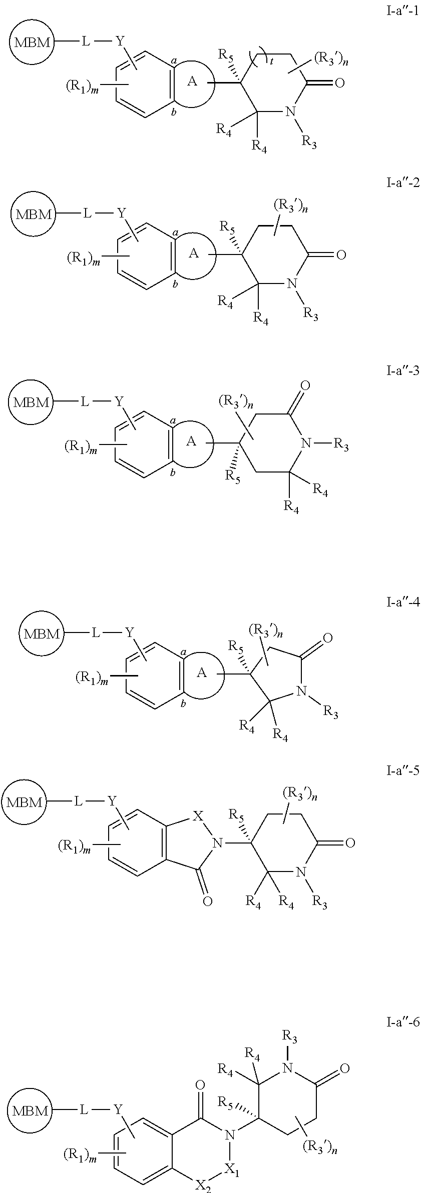

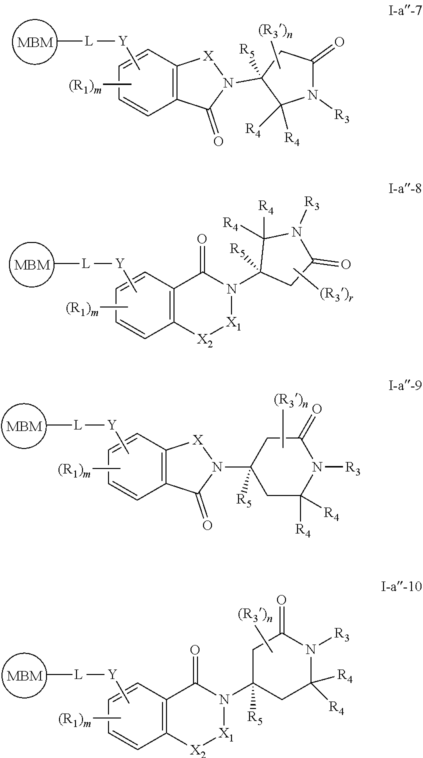

or a pharmaceutically acceptable salt thereof, wherein L and MBM are as defined above and described in embodiments herein, and wherein:

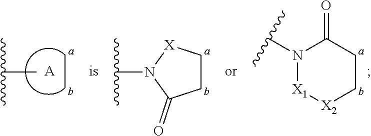

Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R₃)₂;
X₁-X₂ is C(R₃)═N or C(R₃)₂—C(R₃)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆ alkenyl, C(O)—C₃-C₈ cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
each R₃ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or C₁;
each Rₐ independently is H or C₁-C₆ alkyl;
R_{b} is H or tosyl;
t is 0 or 1;
m is 0, 1, 2 or 3; and
n is 0, 1 or 2.

In some embodiments, LBM is

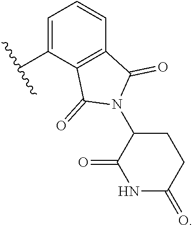 [SMILES: *c1cccc2c1C(=O)N(C1CCC(=O)NC1=O)C2=O]

In some embodiments, LBM is

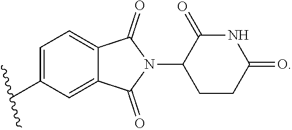 [SMILES: *c1ccc2c(c1)C(=O)N(C1CCC(=O)NC1=O)C2=O]

In some embodiments, LBM is

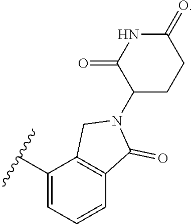 [SMILES: *c1cccc2c1CN(C1CCC(=O)NC1=O)C2=O]

In some embodiments, LBM is

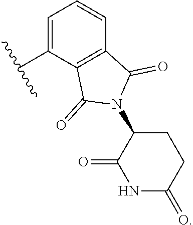 [SMILES: *c1cccc2c1C(=O)N([C@H]1CCC(=O)NC1=O)C2=O]

In some embodiments, LBM is

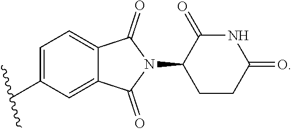 [SMILES: *c1ccc2c(c1)C(=O)N([C@@H]1CCC(=O)NC1=O)C2=O]

In some embodiments, LBM is

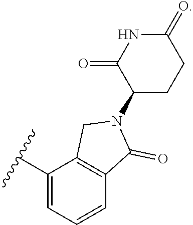 [SMILES: *c1cccc2c1CN([C@@H]1CCC(=O)NC1=O)C2=O]

In some embodiments, LBM is

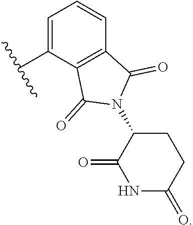 [SMILES: *c1cccc2c1C(=O)N([C@@H]1CCC(=O)NC1=O)C2=O]

In some embodiments, LBM is

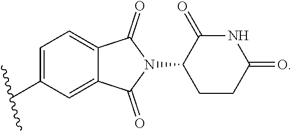 [SMILES: *c1ccc2c(c1)C(=O)N([C@H]1CCC(=O)NC1=O)C2=O]

In some embodiments, LBM is

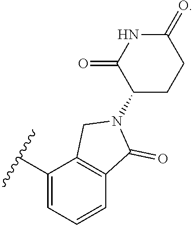 [SMILES: *c1cccc2c1CN([C@H]1CCC(=O)NC1=O)C2=O]

In certain embodiments, the present invention provides a compound of formula I, wherein LBM is a cereblon E3 ubiquitin ligase binding moiety thereby forming a compound of formula I-b:

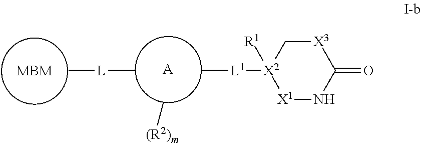

or a pharmaceutically acceptable salt thereof, wherein L and MBM are as defined above and described herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

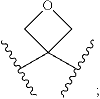 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a bi- or tricyclic ring selected from

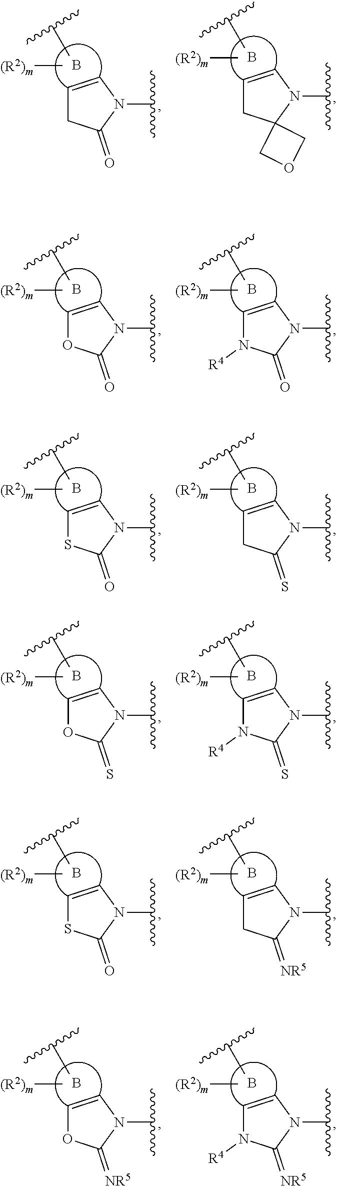

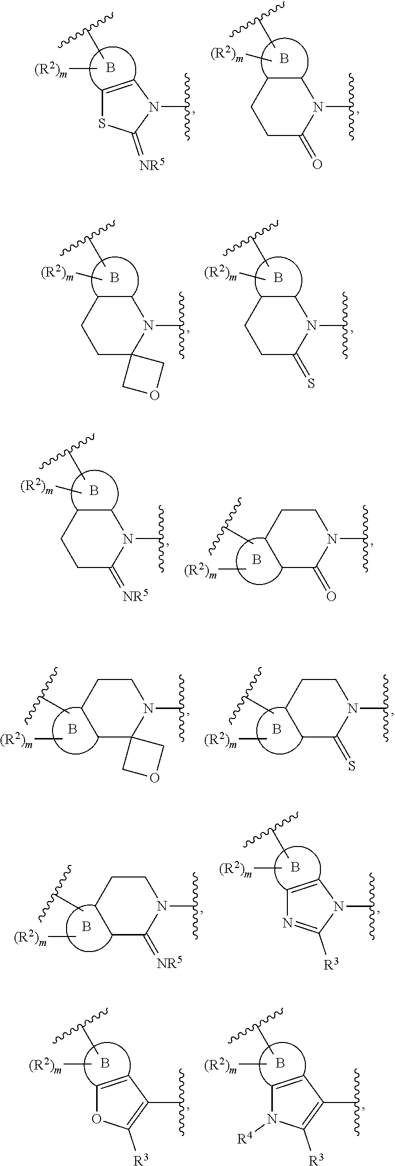

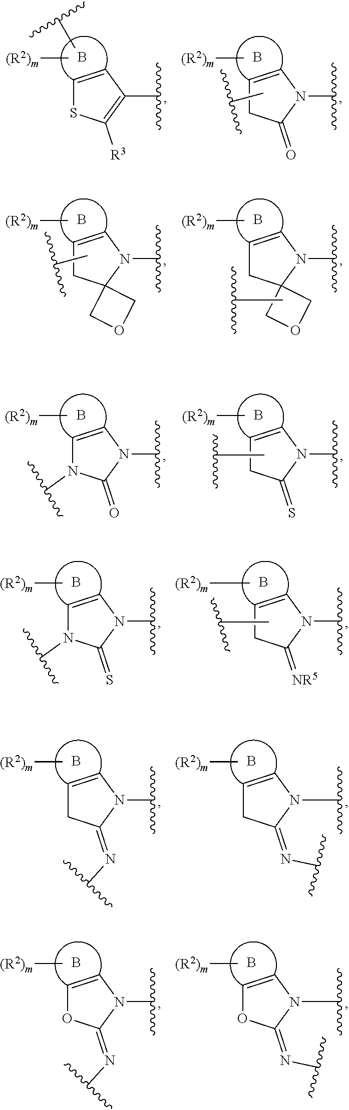

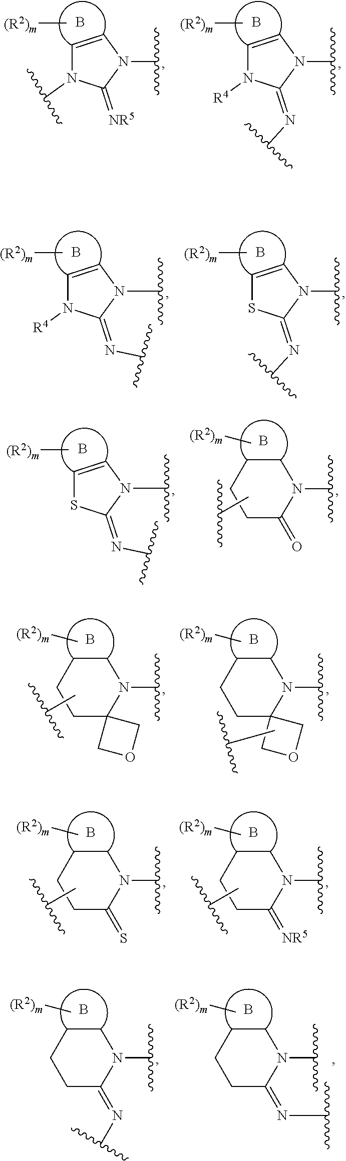

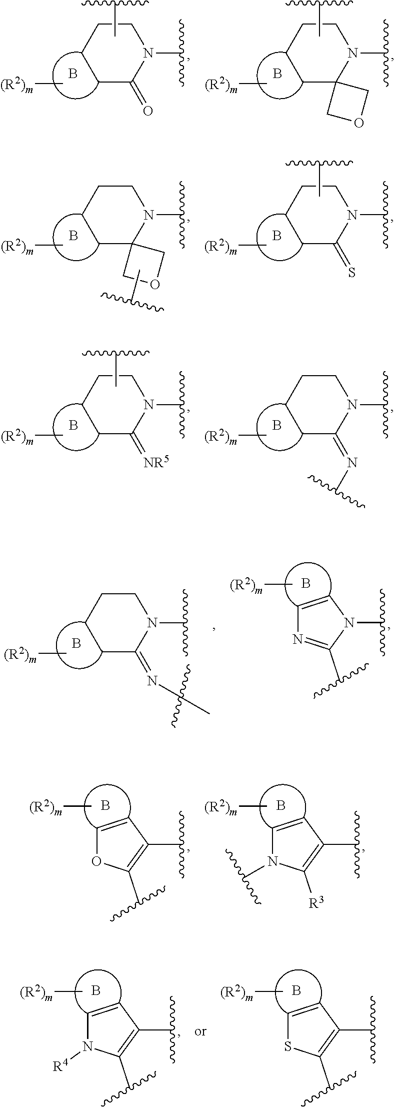

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur.

Where a point of attachment of —(R²)ₘ is depicted on Ring B, it is intended, and one of ordinary skill in the art would appreciate, that the point of attachment of —(R²)ₘ may be on Ring A and may also be at any available carbon or nitrogen atom on Ring A including the ring to which Ring B is fused. Where —R² is attached to a nitrogen atom bound to R⁴ or R⁵, R⁴ or R⁵ is absent and —R² takes the place of the R⁴ or R⁵ group. Where —R² is attached to a carbon atom bound to R³, R³ is absent and —R² takes the place of the R³ group.

In some embodiments, a compound of formula I-b above is provided as a compound of formula I-b-1 or formula I-b-2:

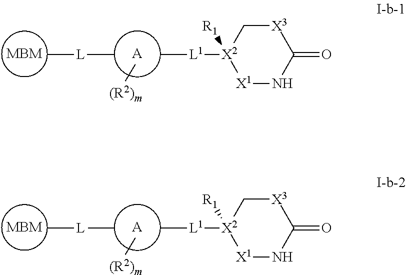

or a pharmaceutically acceptable salt thereof, wherein:
each of MBM, Ring A, L, L¹, R¹, R², X¹, X², X³, and m is as defined above.

In some embodiments, a compound of formula I-b above is provided as a compound of formula I-b-3:

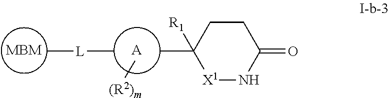

or a pharmaceutically acceptable salt thereof, wherein:
each of MBM, Ring A, L, R¹, R², X¹, and m is as defined above.

In certain embodiments, the present invention provides a compound of formula I as a compound of formula I-aa-1:

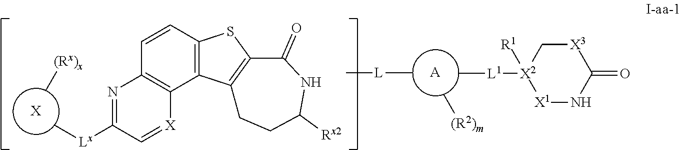

or a pharmaceutically acceptable salt thereof, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

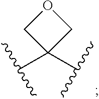 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
   each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bi- or tricyclic ring selected from

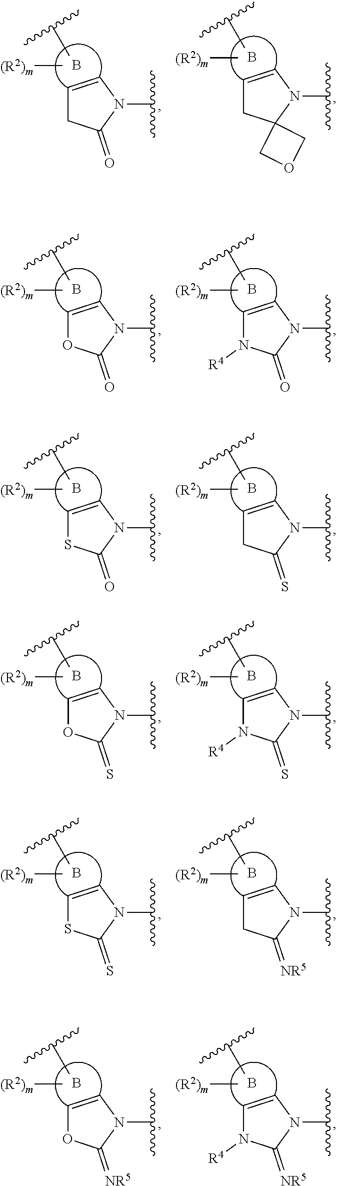

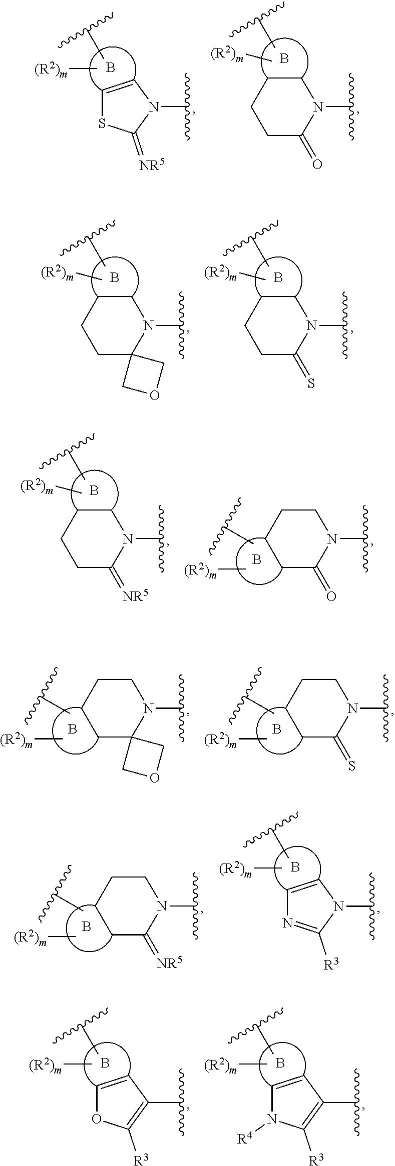

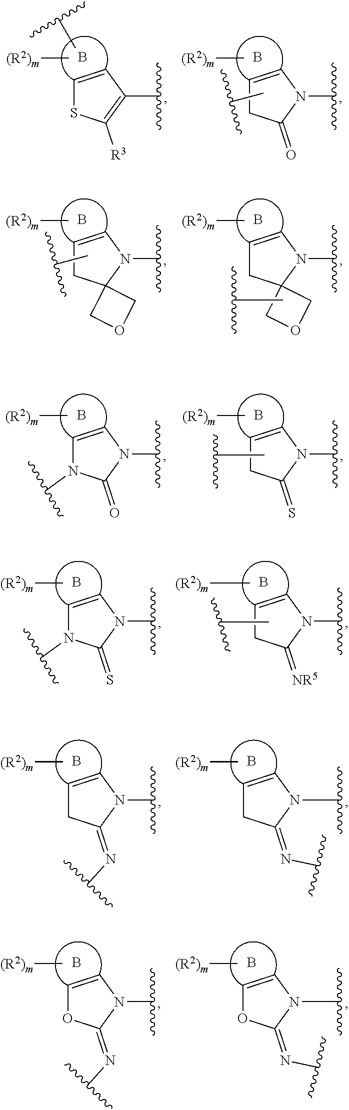

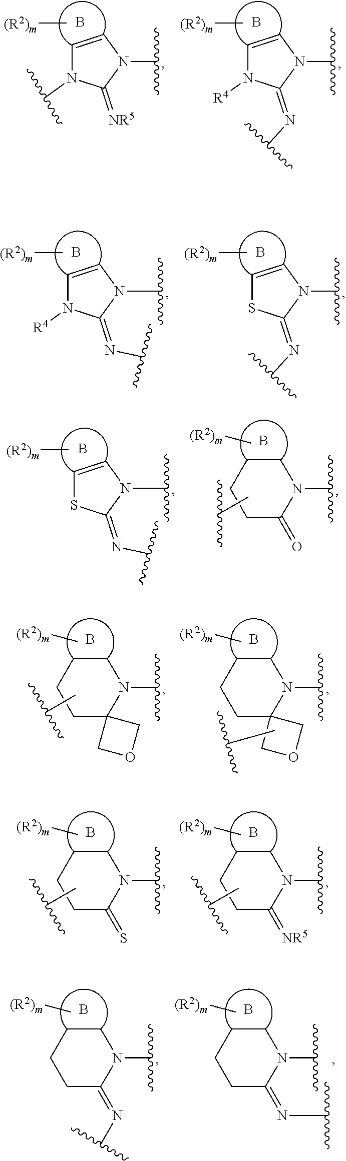

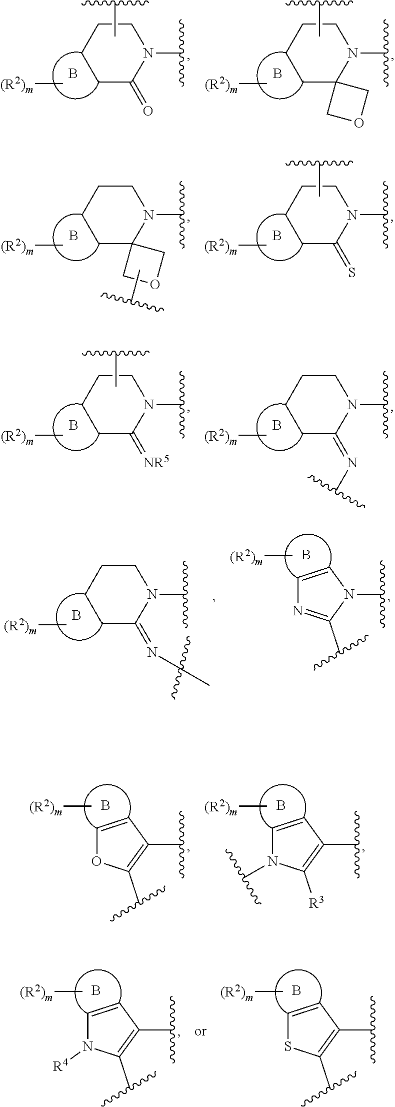

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R₄ is independently hydrogen, R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
L¹ is a covalent bond or a bivalent, saturated or partially unsaturated, straight or branched C₁₋₅₀ hydrocarbon chain, wherein 0-10 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

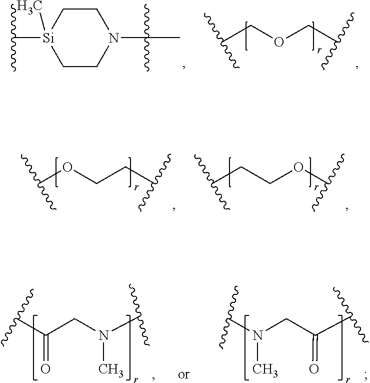

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
Ring X is an optionally substituted ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
X is an optionally substituted carbon or nitrogen atom;
Y is —O—, —S—, or —N(R^{x1})—;
R^{x} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)N(R)₂, —C(O)NROR, —CR₂NRC(O)R, —CR₂NRC(O)N(R)₂, —OC(O)R, —OC(O)N(R)₂, —OP(O)R₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} is a covalent bond or a bivalent, saturated or partially unsaturated, straight or branched C₁₋₅ hydrocarbon chain, wherein 0-3 methylene units of L^{x} are independently replaced by —O—, —NR—, —CRF—, —CF₂—, —C(O)—, —S—, —S(O)—, or —S(O)₂—;
R^{x1} and R^{x2} are, independently, hydrogen or an optionally substituted C₁₋₆ aliphatic; and
x is 0, 1, 2, 3, or 4.

In some embodiments, the present invention provides a compound of formula I-aa-1, wherein X and X² are carbon atoms, Y is —N(R^{x1})—, L¹ is a bond, and X³ is —CH₂—, to provide a compound of formula I-aa-2:

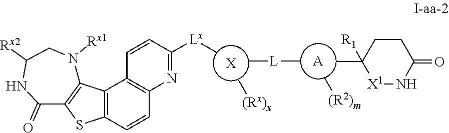

or a pharmaceutically acceptable salt, wherein each of Ring A, Ring X, L, L^{x}, X¹, R^{x}, R^{x1}, R^{x2}, R¹, R², x, and m is as defined above and described in embodiments herein, both singly and in combination.

In some embodiments, the present invention provide a compound of formula I-aa-2, wherein Ring X is piperidinylenyl, piperazinylenyl, phenylenyl, or pyridinylenyl.

In some embodiments, the present invention provides a compound of formula I-aa-1, wherein Ring X is piperidinylenyl, X and X² are carbon atoms, Y is —N(R^{x1})—, L¹ and L^{x} are bonds, and X³ is —CH₂—, to provide a compound of formula I-aa-3:

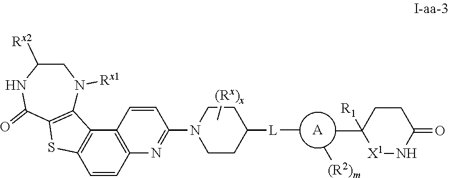

or a pharmaceutically acceptable salt, wherein each of Ring A, L, X¹, R^{x}, R^{x1}, R^{x2}, R¹, R², x, and m is as defined above and described in embodiments herein, both singly and in combination.

In some embodiments, the present invention provides a compound of formula I-aa-1, wherein Ring X is piperazinylenyl, X and X² are carbon atoms, Y is —N(R^{x1})—, L¹ and L^{x} are bonds, and X³ is —CH₂—, to provide a compound of formula I-aa-4:

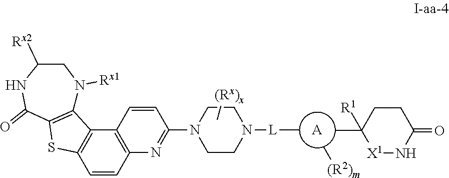

or a pharmaceutically acceptable salt, wherein each of Ring A, L, X¹, R^{x}, R^{x1}, R^{x2}, R¹, R², x, and m is as defined above and described in embodiments herein, both singly and in combination.

In some embodiments, the present invention provides a compound of formula I-aa-1, wherein Ring X is para-fused phenylenyl, X and X² are carbon atoms, Y is —N(R^{x1})—, L¹ and L^{x} are bonds, and X³ is —CH₂—, to provide a compound of formula I-aa-5:

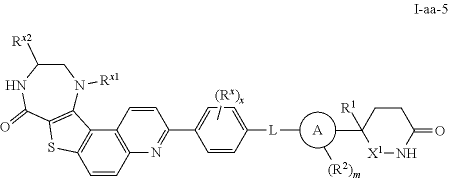

or a pharmaceutically acceptable salt, wherein each of Ring A, L, X¹, R^{x}, R^{x1}, R^{x2}, R¹, R², x, and m is as defined above and described in embodiments herein, both singly and in combination.

In some embodiments, the present invention provides a compound of formula I-aa-1, wherein Ring X is meta-fused phenylenyl, X and X² are carbon atoms, Y is —N(R^{x1})—, L¹ and L^{x} are bonds, and X³ is —CH₂—, to provide a compound of formula I-aa-6:

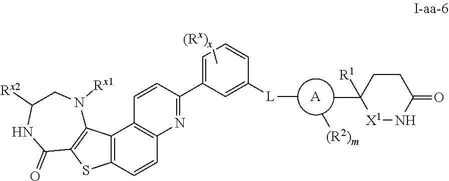

or a pharmaceutically acceptable salt, wherein each of Ring A, L, X¹, R^{x}, R^{x1}, R^{x2}, R¹, R², x, and m is as defined above and described in embodiments herein, both singly and in combination.

In some embodiments, the present invention provides a compound of formula I-aa-1, wherein Ring X is para-fused pyridinylenyl, X and X² are carbon atoms, Y is —N(R^{x1})—, L¹ and L^{x} are bonds, and X³ is —CH₂—, to provide a compound of formula I-aa-7:

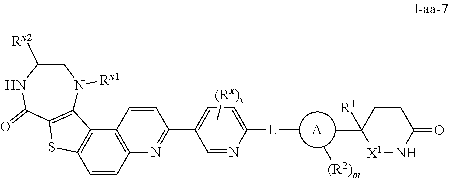

or a pharmaceutically acceptable salt, wherein each of Ring A, L, X¹, R^{x}, R^{x1}, R^{x2}, R¹, R², x, and m is as defined above and described in embodiments herein, both singly and in combination.

In certain embodiments, the present invention provides a compound of formula I as a compound of formula I-bb-3:

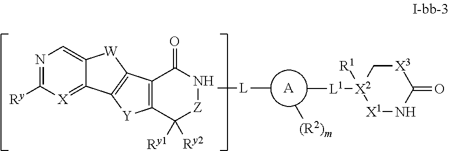

or a pharmaceutically acceptable salt, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

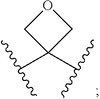 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bi- or tricyclic ring selected from

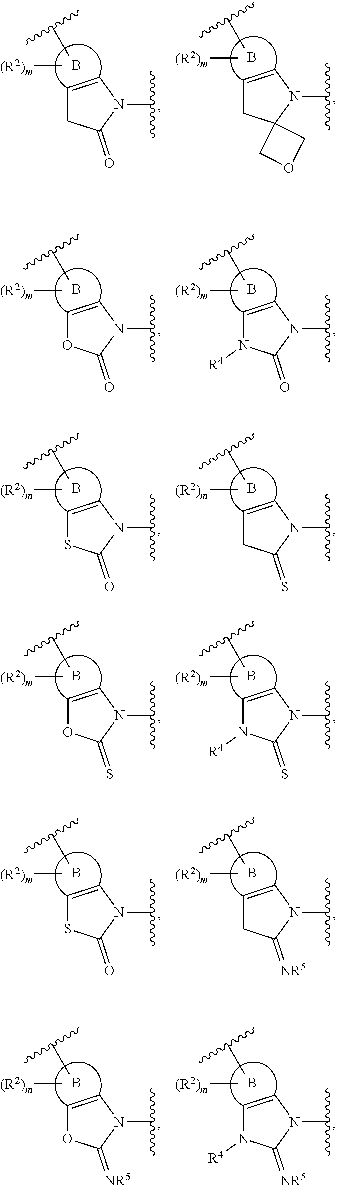

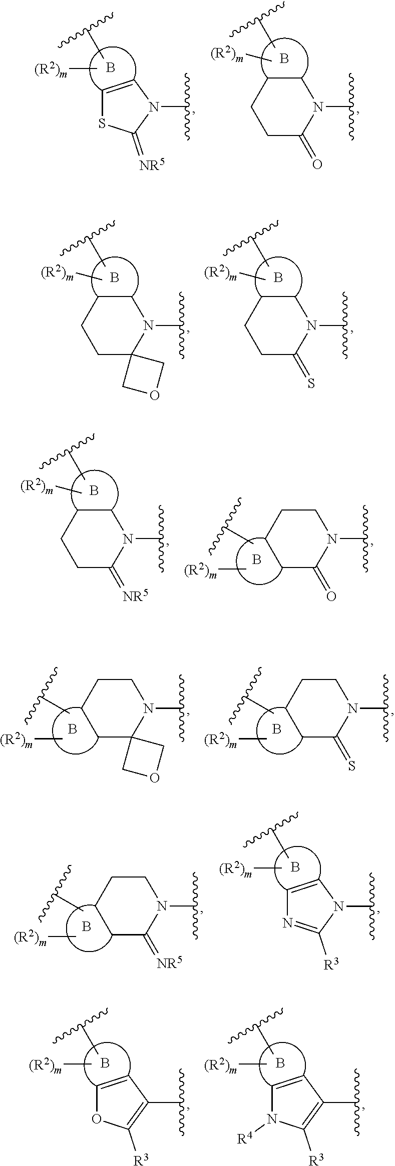

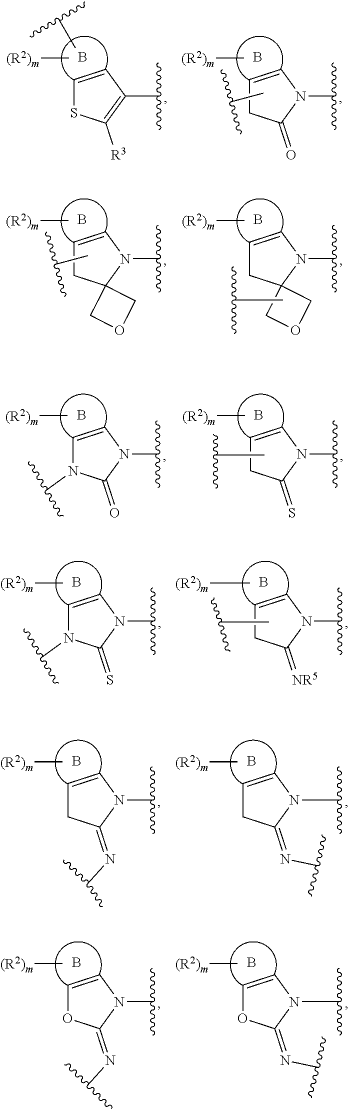

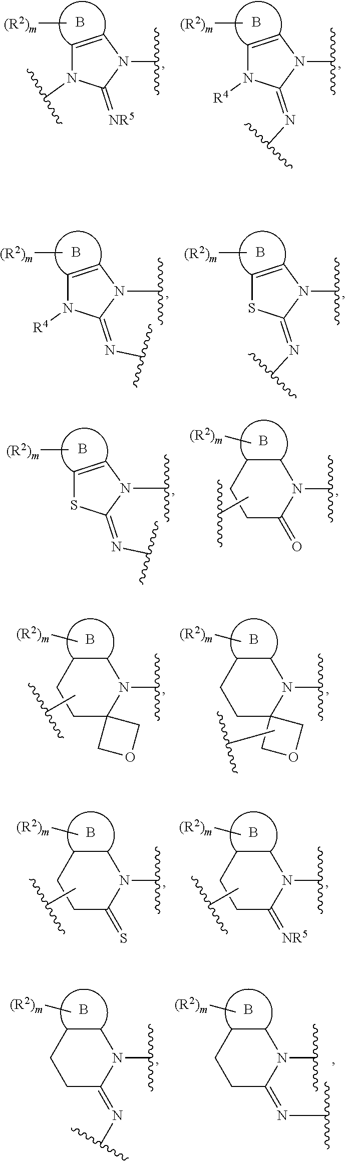

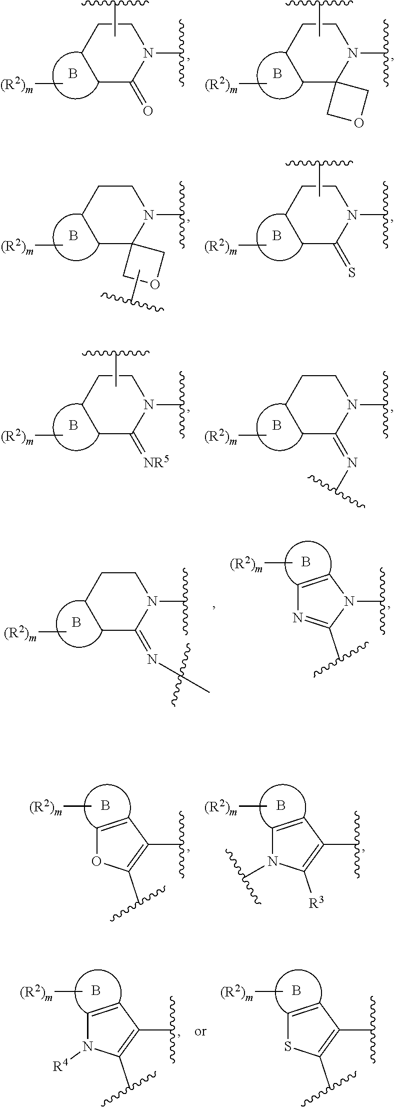

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R⁴ is independently hydrogen, R⁶, halogen, —CN, —NO₂, —OR, SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
L¹ is a covalent bond or a C₁_3 bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
L is a covalent bond or a bivalent, saturated or partially unsaturated, straight or branched C₁₋₅₀ hydrocarbon chain, wherein 0-10 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

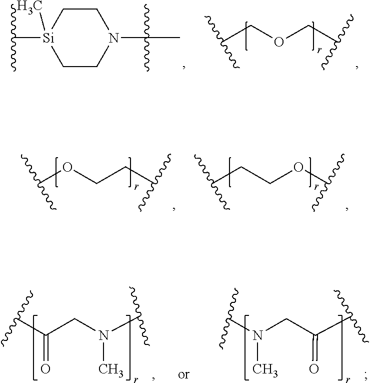

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
R^{y} is hydrogen, halogen, —Y¹—R, an optionally substituted C₁₋₆ aliphatic,

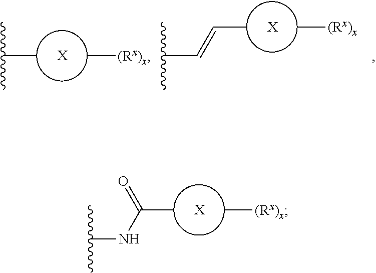

Ring X is an optionally substituted ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{x} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)N(R)₂, —C(O)NROR, —CR₂NRC(O)R, —CR₂NRC(O)N(R)₂, —OC(O)R, —OC(O)N(R)₂, —OP(O)R₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X is an optionally substituted carbon or nitrogen atom;
Y and Y¹ are, independently, a bivalent group selected from —O—, —S— and —NR—;
W is a bivalent group selected from —CH₂—, —CH₂CH₂—, —CH₂CH₂CH₂— and —CH═CH—;
Z is a bivalent group selected from a bond and —C(R^{y3})(R^{y4})—;
R^{y1} and R^{y2} are, independently, hydrogen or taken together with their intervening atoms to form an optionally substituted 4-7 membered saturated or partially unsaturated carbocyclic or heterocyclic ring having 1-3 heteroatoms independently selected from nitrogen, oxygen, and sulfur;
R^{y3} and R^{y4} are, independently, hydrogen or taken together with their intervening atoms to form an optionally substituted 4-7 membered saturated or partially unsaturated carbocyclic or heterocyclic ring having 1-3 heteroatoms independently selected from nitrogen, oxygen, and sulfur; and
x is 0, 1, 2, 3, or 4.

In some embodiments, the present invention provides a compound of formula I-bb-3, wherein X² is a carbon atom, L¹ is a bond, X³ is —CH₂—, and R^{y} is

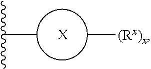

to provide a compound of formula I-bb-4:

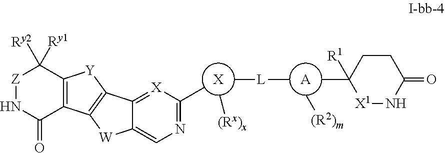

or a pharmaceutically acceptable salt, wherein each of Ring A, Ring X, L, W, X, X¹, Y, Z, R^{x}, R^{y1}, R^{y2}, R¹, R₂, m and x is as defined above and described in embodiments herein, both singly and in combination.

In certain embodiments, the present invention provides a compound of formula I as a compound of formula I-cc-1:

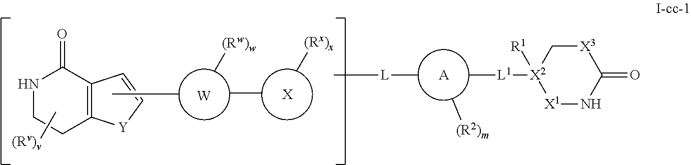

or a pharmaceutically acceptable salt, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

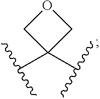 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bi- or tricyclic ring selected from

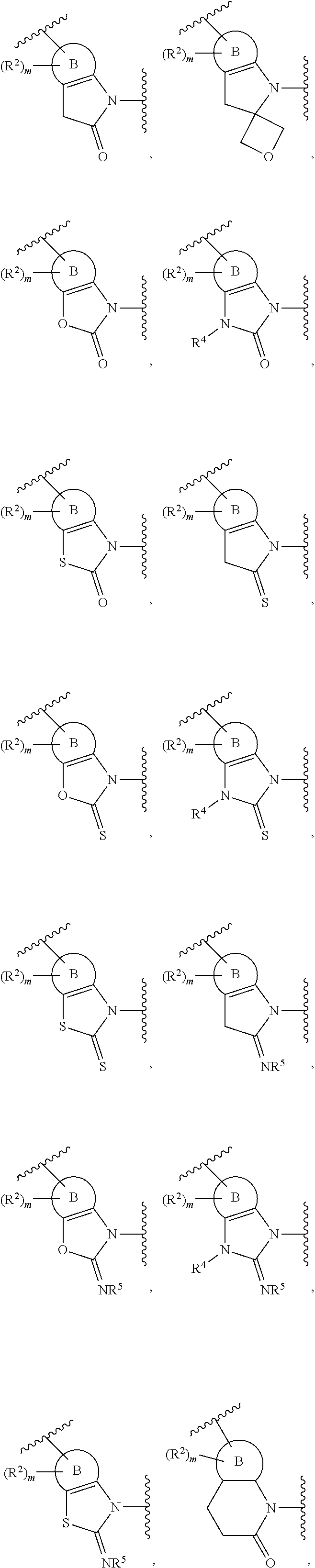

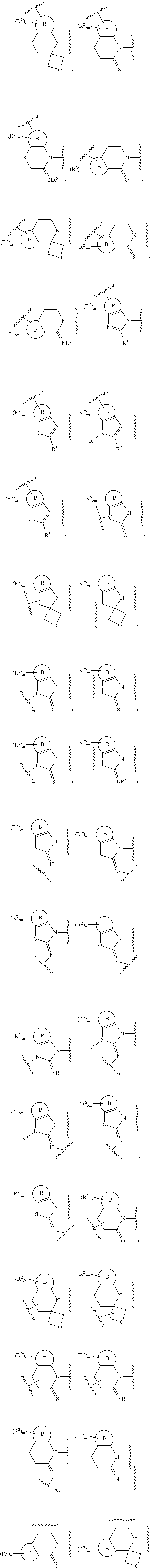

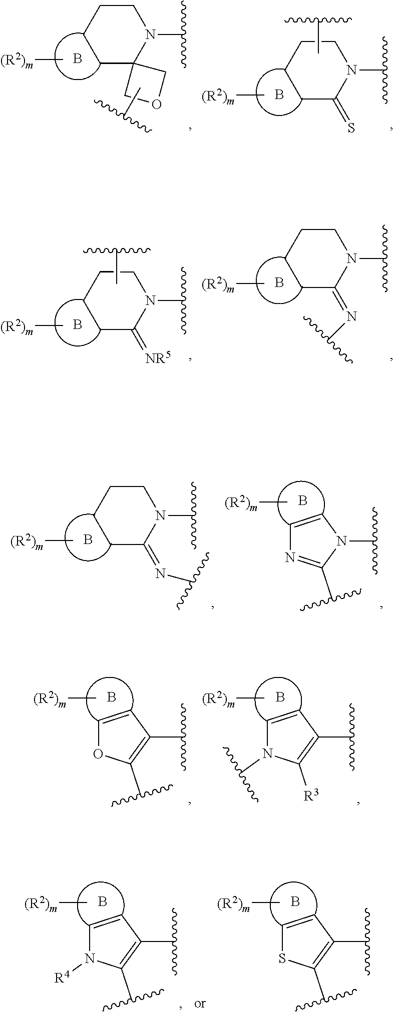

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R⁴ is independently hydrogen, R⁶, halogen, —CN, —NO₂, —OR, SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
L is a covalent bond or a bivalent, saturated or partially unsaturated, straight or branched C₁₋₅₀ hydrocarbon chain, wherein 0-10 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

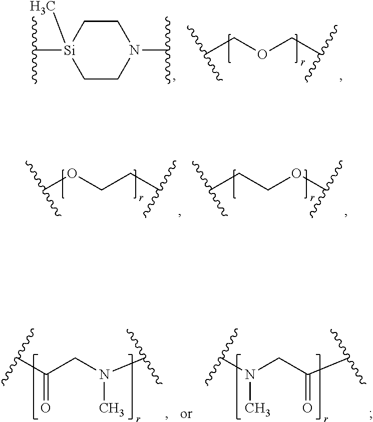

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9,or 10;
Y is —CH═CH—, —NH—, —O—, or —S—;
Ring W is a bivalent ring selected from phenylenyl, naphthylenyl, a 5-10 membered heteroarylenyl containing 1-4 heteroatoms independently selected from nitrogen, oxygen, and sulfur, and a 5-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur;
Ring X is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{v}, R^{w}, and R^{x} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R; or:
   two R^{v} groups on the same or different atoms are optionally taken together with their intervening atoms to form an optionally substituted 3-6 membered saturated or partially unsaturated carbocyclic ring or heterocyclic ring with 1-2 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-9 membered heteroaryl ring having 1-4 heteroatoms independently selected from nitrogen, oxygen, and sulfur;
v is 0, 1, 2, 3, or 4;
w is 0, 1, 2, 3, or 4; and
x is 0, 1, 2, 3, or 4.

In some embodiments, the present invention provides a compound of formula I-cc-1, wherein Y is —NH—, X² is a carbon atom, L¹ is a bond, and X³ is —CH₂—, to provide a compound of formula I-cc-2:

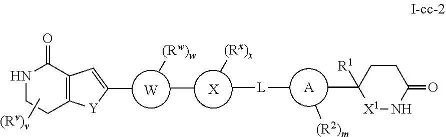

or a pharmaceutically acceptable salt, wherein each of Ring A, Ring, W, Ring X, L, X¹, R^{w}, R^{x}, R¹, R², w, x, and m is as defined above and described in embodiments herein, both singly and in combination.

In some embodiments, the present invention provide a compound of formula I-cc-2, wherein Ring W is a meta-fused phenylenyl, pridinylenyl, or primidinylenyl.

In some embodiments, the present invention provides a compound of formula I-cc-1, wherein Y is —NH—, Ring W is a meta-fused pridinylenyl, X² is a carbon atom, L¹ is a bond, and X³ is —CH₂—, to provide a compound of formula I-cc-3:

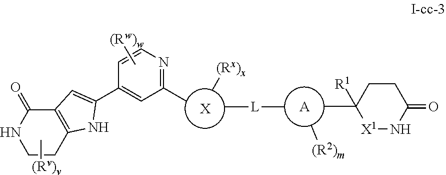

or a pharmaceutically acceptable salt, wherein each of Ring A, Ring X, L, X¹, R^{w}, R^{x}, R¹, R², w, x, and m is as defined above and described in embodiments herein, both singly and in combination.

In some embodiments, the present invention provides a compound of formula I-cc-1, wherein Y is —NH—, Ring W is a meta-fused primidinylenyl, X² is a carbon atom, L¹ is a bond, and X³ is —CH₂—, to provide a compound of formula I-cc-4:

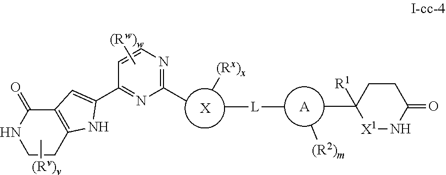

or a pharmaceutically acceptable salt, wherein each of Ring A, Ring X, L, X¹, R^{w}, R^{x}, R¹, R², w, x, and m is as defined above and described in embodiments herein, both singly and in combination.

In some embodiments, the present invention provides a compound of formula I-cc-1, wherein Y is —NH—, Ring W is a meta-fused pridinylenyl, Ring X is para-fused phenylenyl, X² is a carbon atom, L¹ is a bond, and X³ is —CH₂—, to provide a compound of formula I-cc-5:

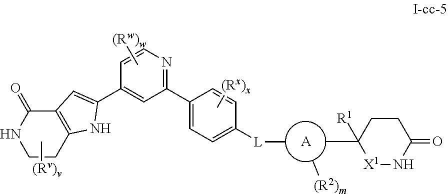

or a pharmaceutically acceptable salt, wherein each of Ring A, L, X¹, R^{w}, R^{x}, R¹, R², w, x, and m is as defined above and described in embodiments herein, both singly and in combination.

In some embodiments, the present invention provides a compound of formula I-cc-1, wherein Y is —NH—, Ring W is a meta-fused primidinylenyl, Ring X is para-fused phenylenyl, X² is a carbon atom, L¹ is a bond, and X³ is —CH₂—, to provide a compound of formula I-cc-6:

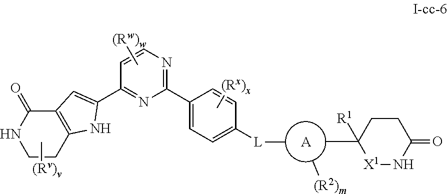

or a pharmaceutically acceptable salt, wherein each of Ring A, L, X¹, R^{w}, R^{x}, R¹, R², w, x, and m is as defined above and described in embodiments herein, both singly and in combination.

In certain embodiments, the present invention provides a compound of formula I as a compound of formula I-dd-1:

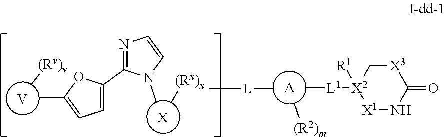

or a pharmaceutically acceptable salt, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

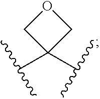 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bi- or tricyclic ring selected from

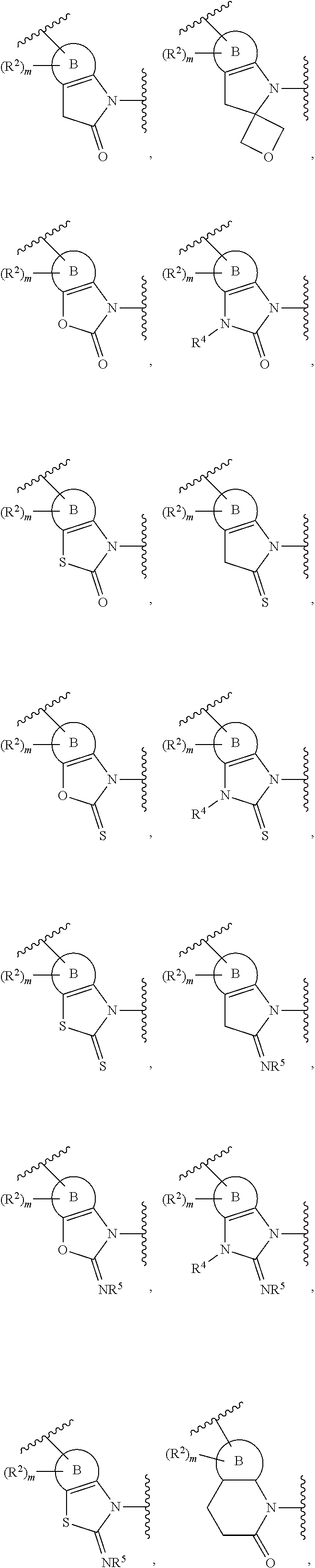

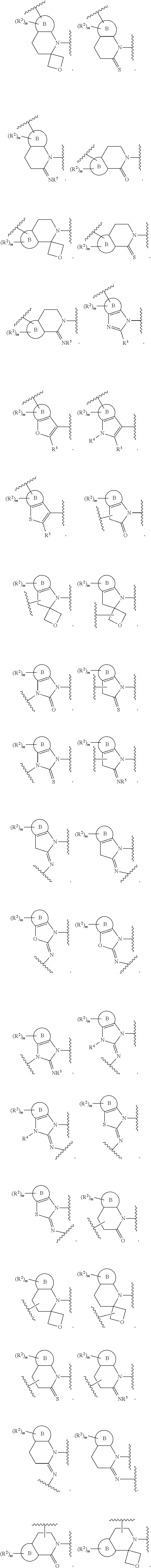

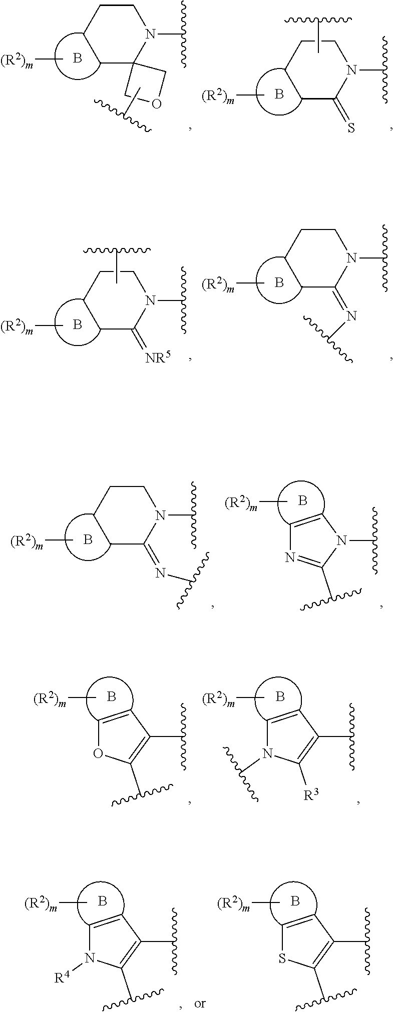

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R⁴ is independently hydrogen, R⁶, halogen, —CN, —NO₂, —OR, SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
L is a covalent bond or a bivalent, saturated or partially unsaturated, straight or branched C₁₋₅₀ hydrocarbon chain, wherein 0-10 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

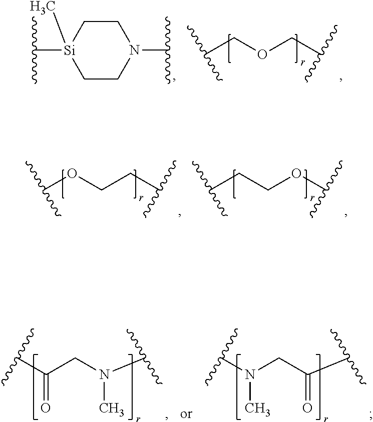

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
Ring V and Ring X are, independently, a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{v} and R^{x} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
v is 0, 1, 2, 3, or 4; and
x is 0, 1, 2, 3, or 4.

In some embodiments, the present invention provides a compound of formula I-dd-1, wherein X² is a carbon atom, L is a bond, and X³ is —CH₂—, to provide a compound of formula I-dd-2:

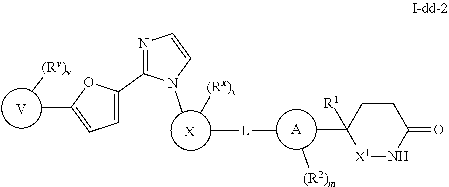

or a pharmaceutically acceptable salt, wherein each of Ring A, Ring V, Ring X, L, X¹, R^{v}, R^{x}, R¹, R² v, x, and m is as defined above and described in embodiments herein, both singly and in combination.

In certain embodiments, the present invention provides a compound of formula I as a compound of formula I-ee-1:

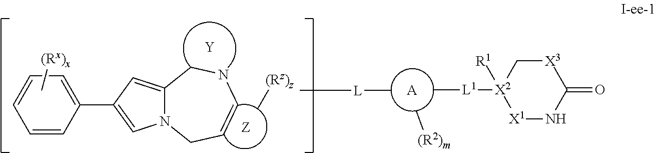

or a pharmaceutically acceptable salt, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

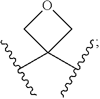 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bi- or tricyclic ring selected from

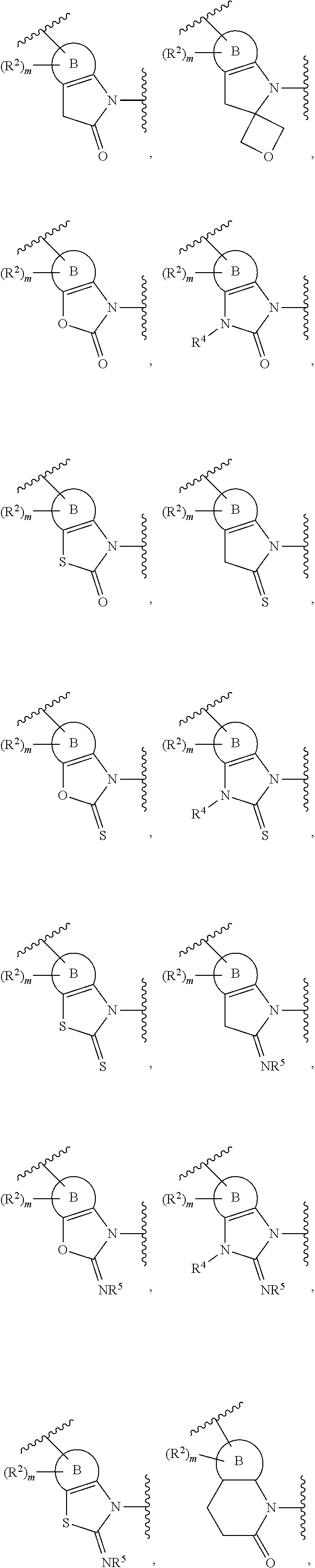

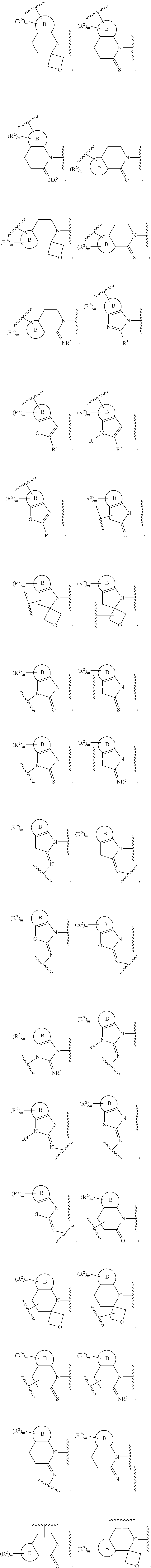

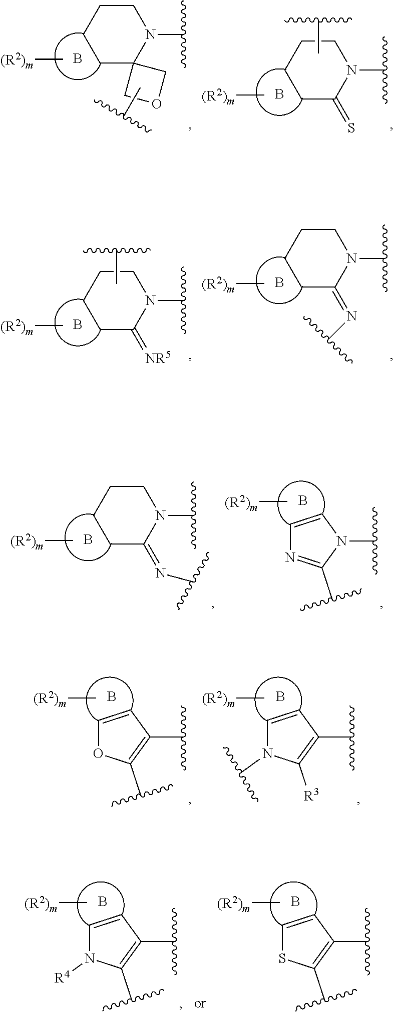

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R⁴ is independently hydrogen, R⁶, halogen, —CN, —NO₂, —OR, SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
L is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
L is a covalent bond or a bivalent, saturated or partially unsaturated, straight or branched C₁₋₅₀ hydrocarbon chain, wherein 0-10 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

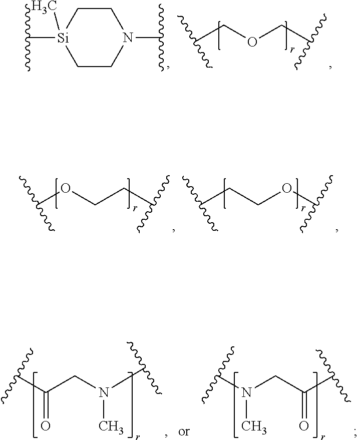

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
   r is 0, 1, 2, 3, 4, 5, 6, 7, 8, 9, or 10;
Ring Y is a fused 5-membered heterocyclic ring with 1-4 heteroatoms independently selected from nitrogen, oxygen, and sulfur;
Ring Z is benzo or a fused 5- to 6-membered heterocyclic ring with 1-4 heteroatoms independently selected from nitrogen, oxygen, and sulfur;
R^{x} and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
x is 0, 1, 2, 3, or 4; and
z is 0, 1, 2, 3, or 4.

In some embodiments, the present invention provides a compound of formula I-ee-1, wherein X² is a carbon atom, L is a bond, and X³ is —CH₂—, to provide a compound of formula I-ee-2:

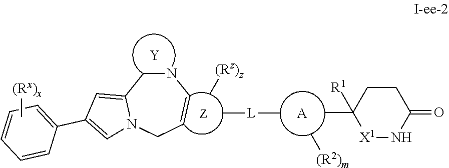

or a pharmaceutically acceptable salt, wherein each of Ring A, Ring, Y, Ring Z, L, X¹, R^{x}, R^{z}, R¹, R², x, z, and m is as defined above and described in embodiments herein, both singly and in combination.

In certain embodiments, the present invention provides a compound of formula I as a compound of formula I-ff-1:

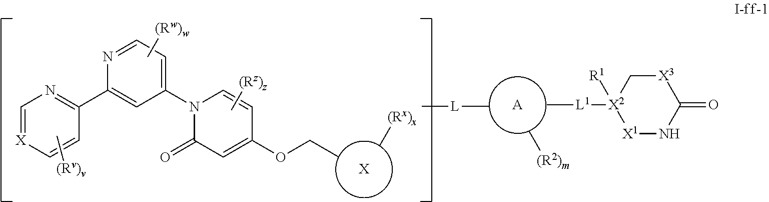

or a pharmaceutically acceptable salt, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

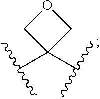 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bi- or tricyclic ring selected from

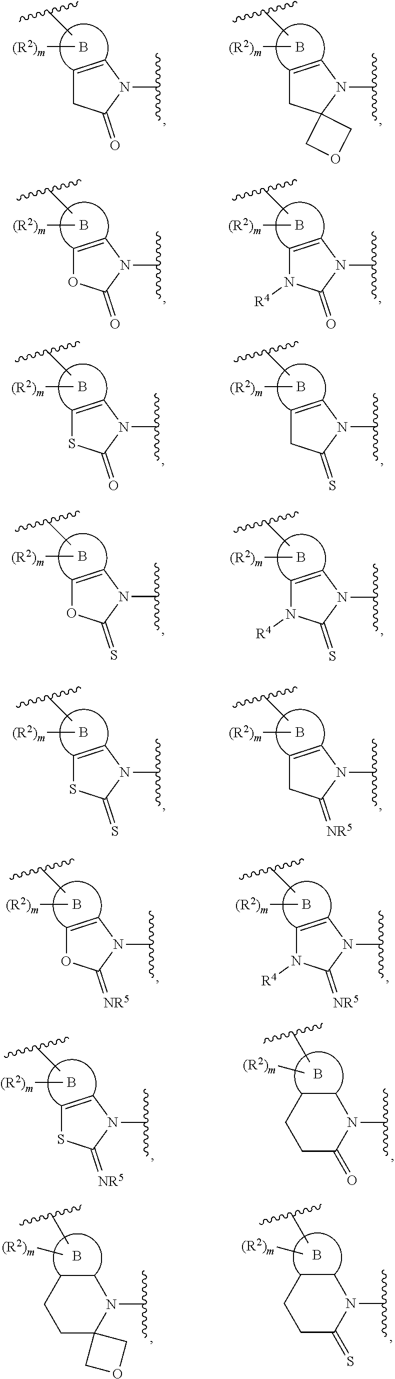

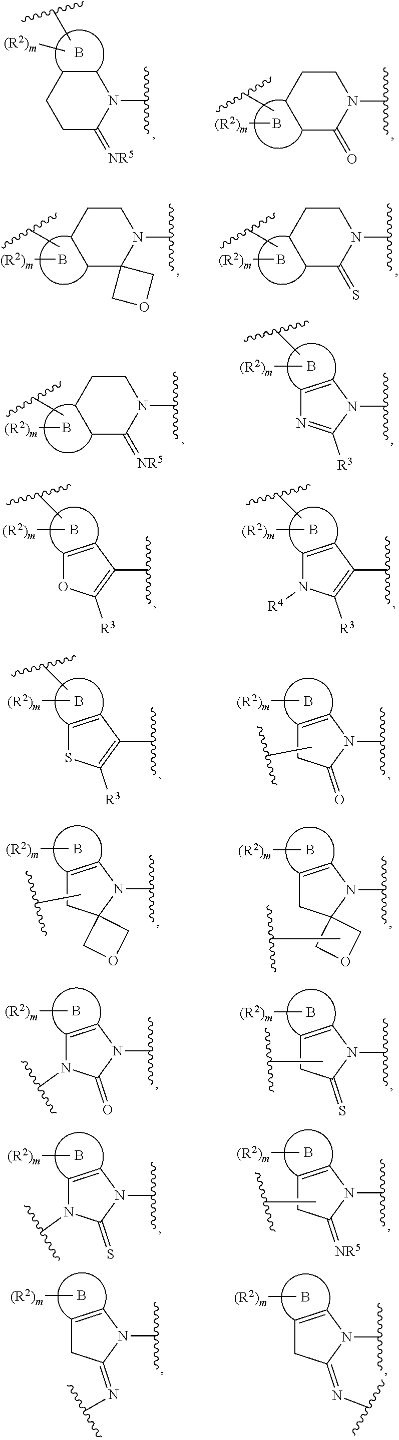

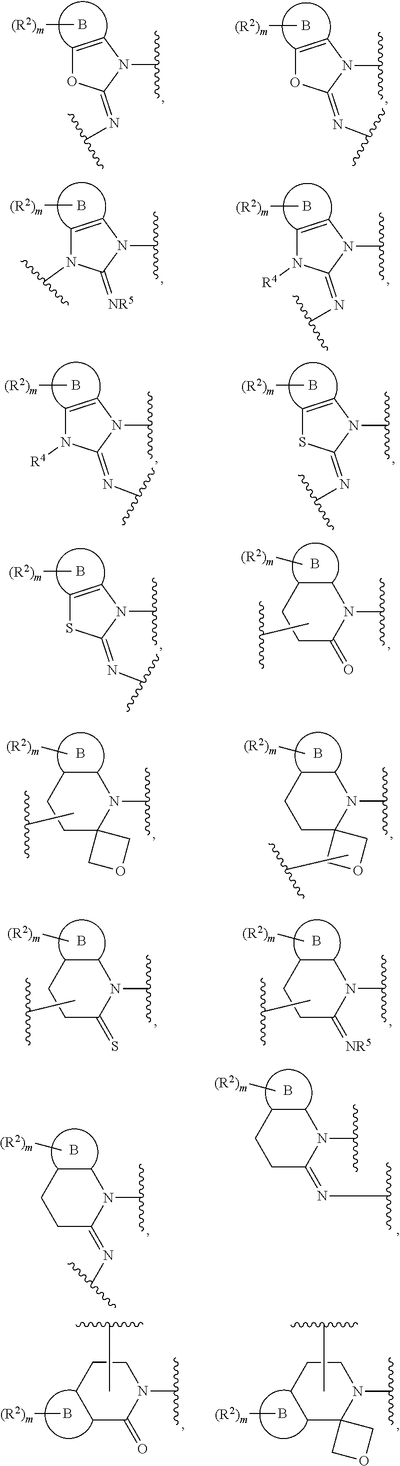

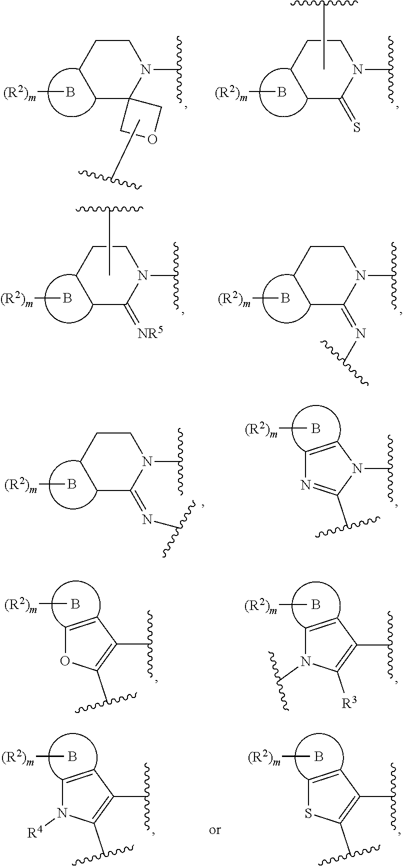

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R⁴ is independently hydrogen, R⁶, halogen, —CN, —NO₂, —OR, SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
L¹ is a covalent bond or a bivalent, saturated or partially unsaturated, straight or branched C₁₋₅₀ hydrocarbon chain, wherein 0-10 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

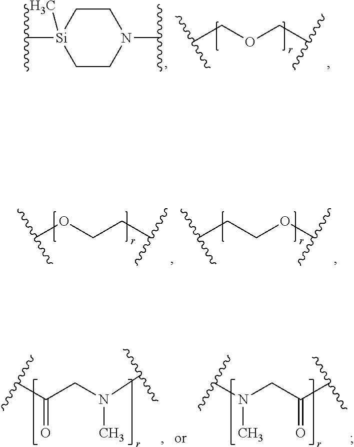

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is a carbon or nitrogen atom;
Ring X is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{v}, R^{w}, R^{x}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
v is 0, 1, 2, 3, or 4;
w is 0, 1, 2, 3, or 4;
x is 0, 1, 2, 3, or 4; and
z is 0, 1, 2, 3, or 4.

In certain embodiments, the present invention provides a compound of formula I, wherein LBM is a cereblon E3 ubiquitin ligase binding moiety thereby forming a compound of formula I-d:

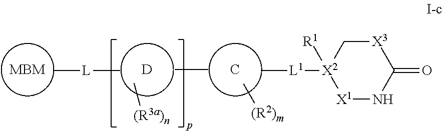

or a pharmaceutically acceptable salt thereof, wherein, L and MBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

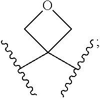 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

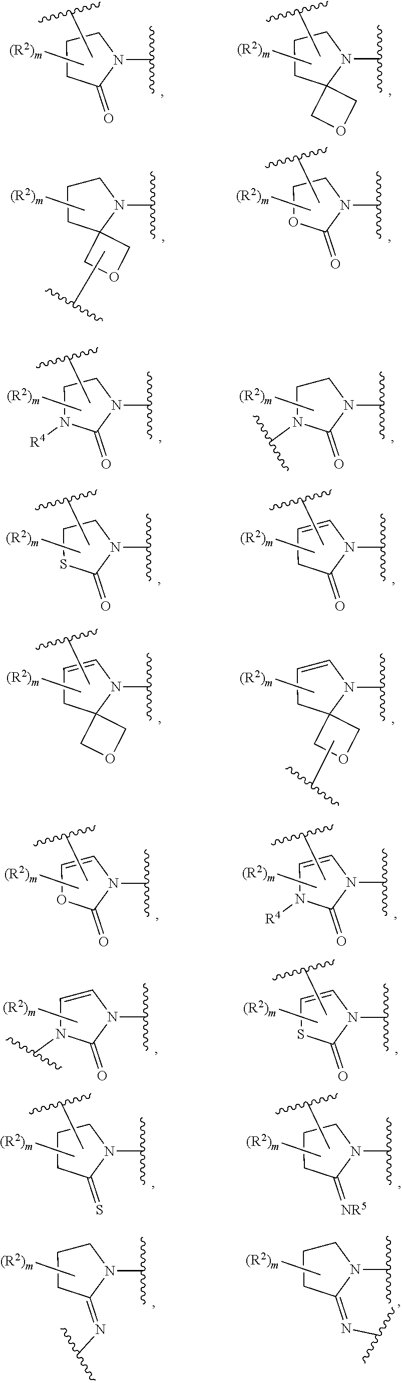

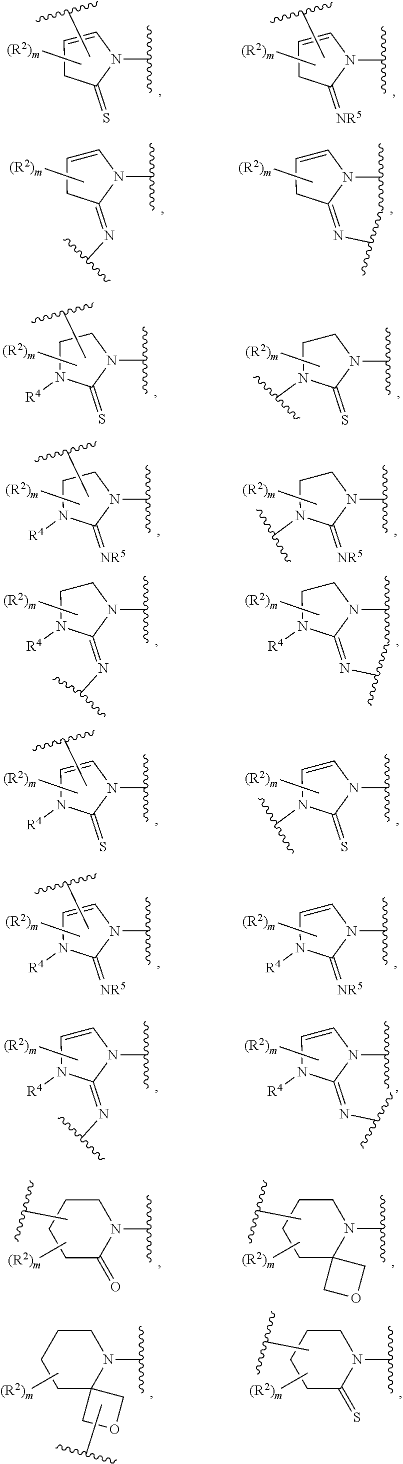

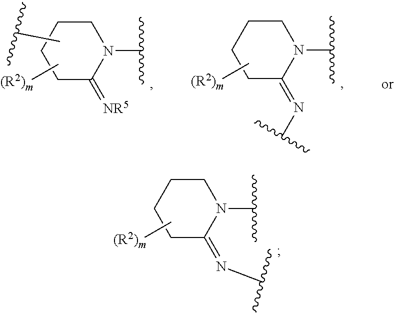

each of R² and R^{3a} is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring D is selected from a 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
each R₄ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
n is 0, 1, 2, 3 or 4;
p is 0 or 1, wherein when p is 0, the bond connecting Ring C and Ring D is connected to

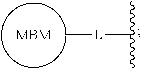

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-c above is provided as a compound of formula I-c-1 or formula I-c-2:

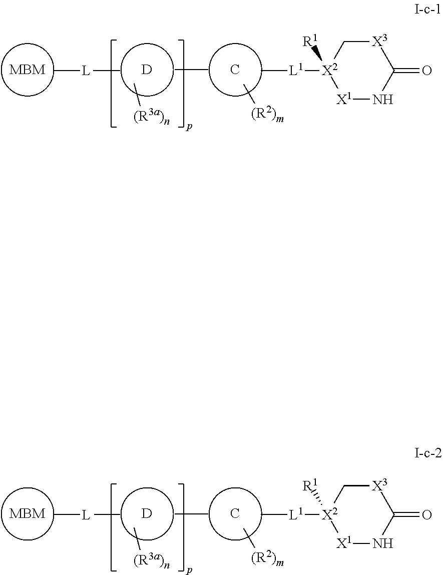

or a pharmaceutically acceptable salt thereof, wherein:
each of MBM, Ring C, Ring D, L, L¹, R¹, R², R^{3a}, X¹, X², X³, n, m, and p is as defined above.

In some embodiments, a compound of formula I-c above is provided as a compound of formula I-c-3:

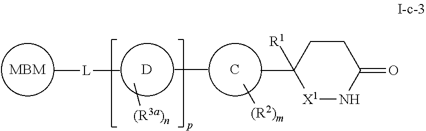

or a pharmaceutically acceptable salt thereof, wherein:
each of MBM, Ring C, Ring D, L, R¹, R², R^{3a}, X¹, n, m, and p is as defined above.

In certain embodiments, the present invention provides a compound of formula I, wherein LBM is a cereblon E3 ubiquitin ligase binding moiety thereby forming a compound of formula I-d:

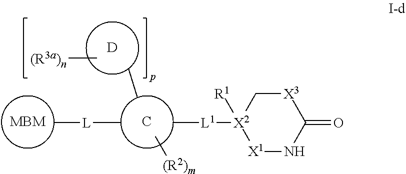

or a pharmaceutically acceptable salt thereof, wherein L and MBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

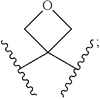 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

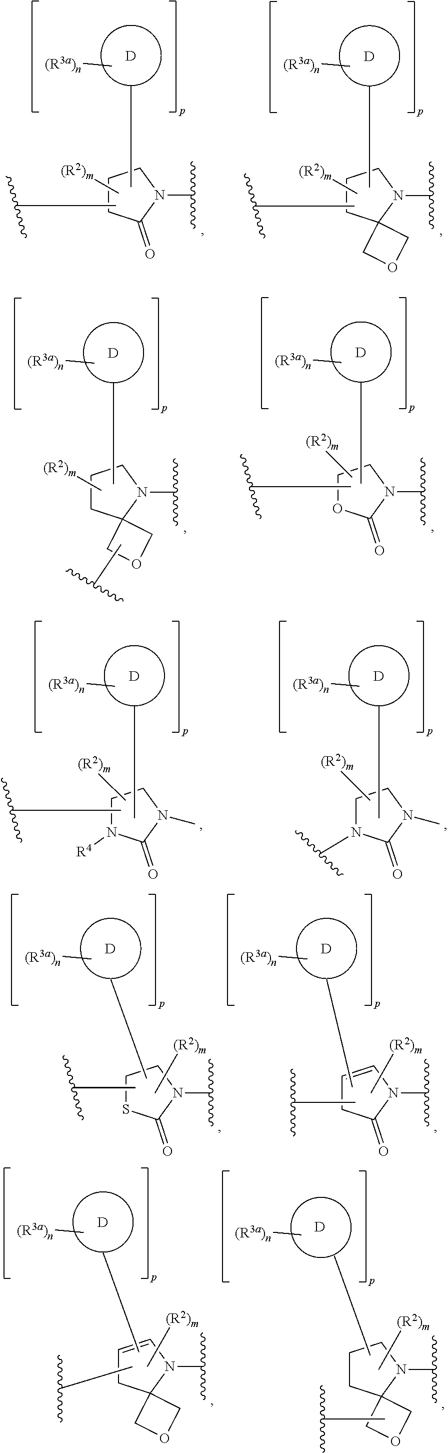

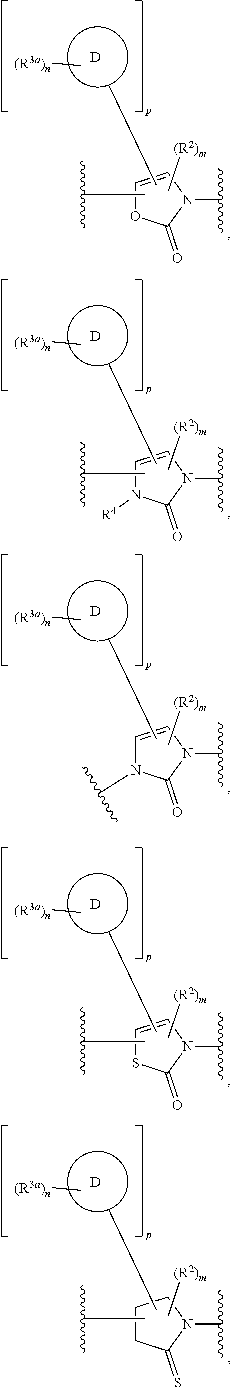

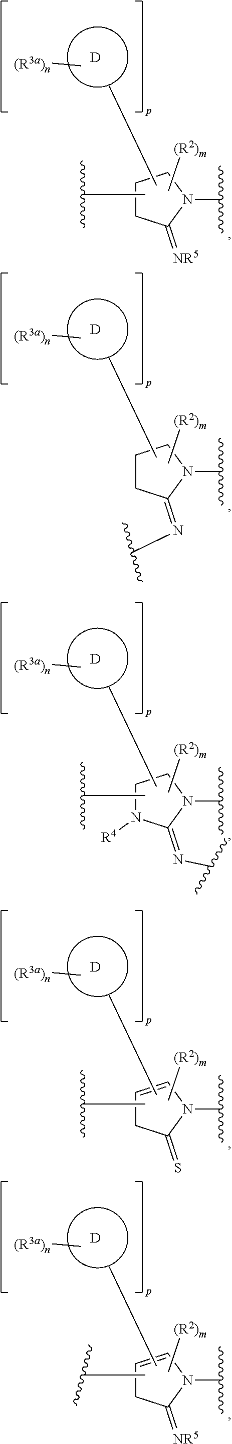

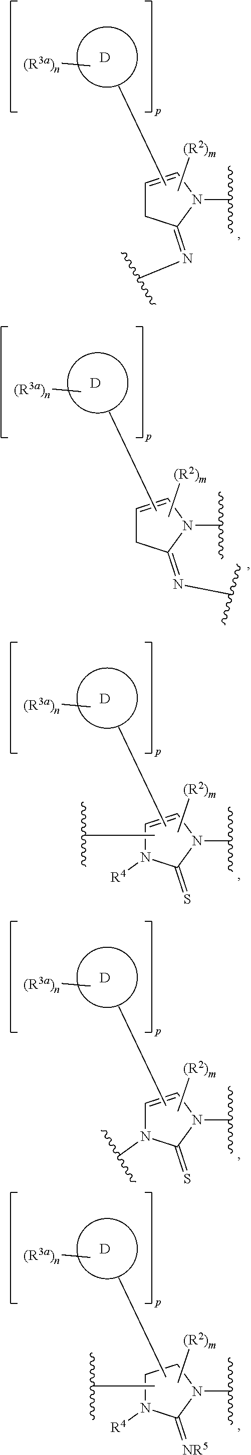

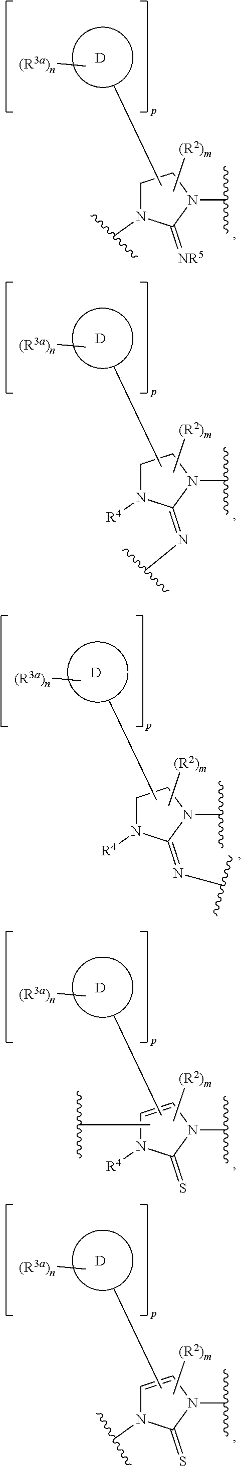

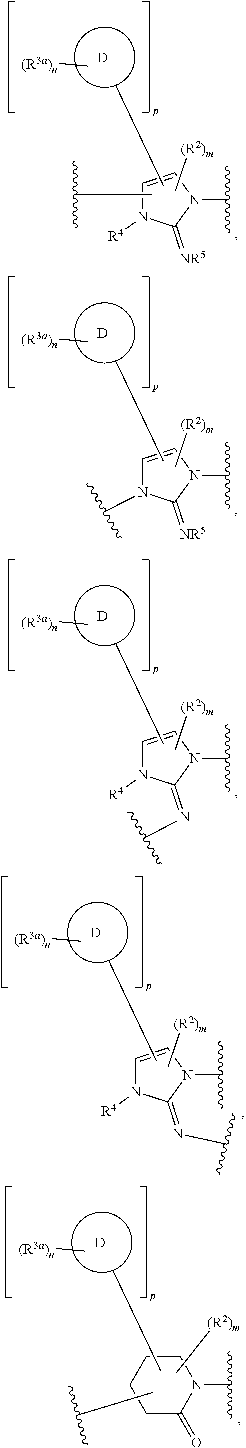

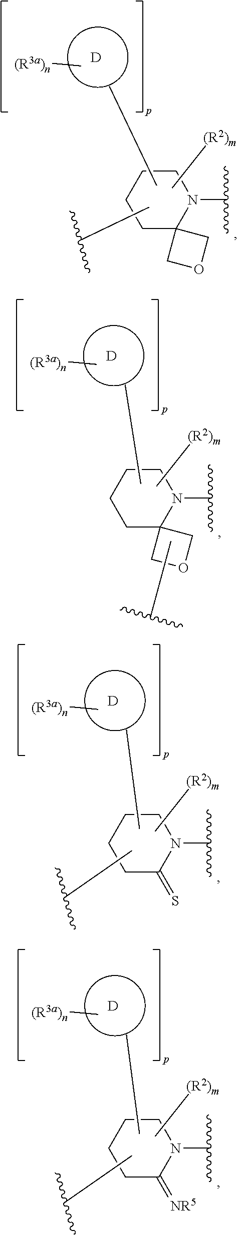

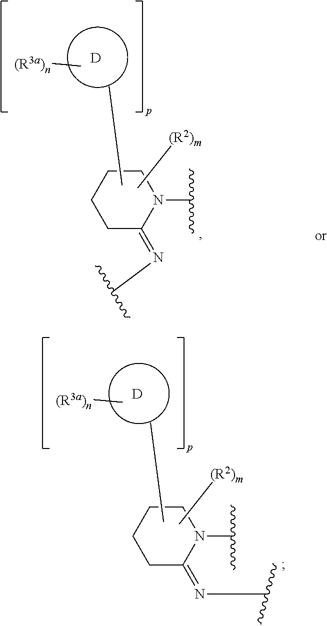

each or R² and R^{3a} is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring D is selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
each R₄ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
n is 0, 1, 2, 3 or 4;
p is 0 or 1; and
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur.

In some embodiments, a compound of formula I-d above is provided as a compound of formula I-d-1 or formula I-d-2:

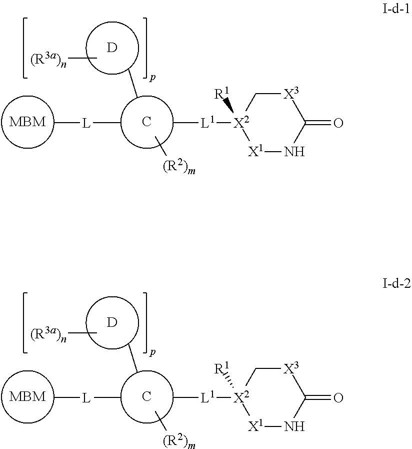

or a pharmaceutically acceptable salt thereof, wherein:
each of MBM, Ring C, Ring D, L, L¹, R¹, R², R^{3a}, X¹, X², X³, m, n, and p is as defined above.

In some embodiments, a compound of formula I-d above is provided as a compound of formula I-d-3:

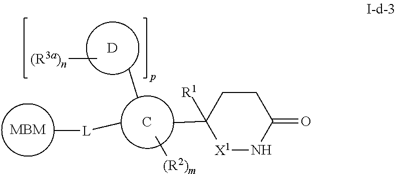

or a pharmaceutically acceptable salt thereof, wherein:
each of MBM, Ring C, Ring D, L, LR¹, R², R^{3a}, X¹, m, n, and p is as defined above.

In certain embodiments, the present invention provides a compound of formula I, wherein LBM is a cereblon E3 ubiquitin ligase binding moiety thereby forming a compound of formula I-e:

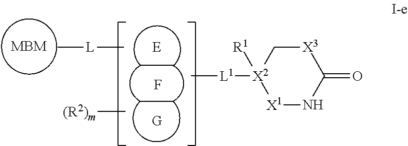

or a pharmaceutically acceptable salt thereof, wherein L and MBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or;

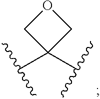 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R² is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—; and
m is 0, 1, 2, 3, 4, 5, 6, 7, 8, 9, 10, 11, 12, 13, 14, 15, or 16.

Where a point of attachment of

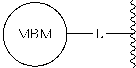

is depicted on Ring E, Ring F, or Ring G, it is intended, and one of ordinary skill in the art would appreciate, that the point of attachment of

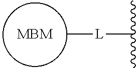

may be on any available carbon or nitrogen atom on Ring E, Ring F, or Ring G, including the ring to which Ring E or Ring G are fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G are fused to Ring F.

Where a point of attachment of

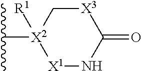

is depicted on Ring E, Ring F, or Ring G, it is intended, and one of ordinary skill in the art would appreciate, that the point of attachment of

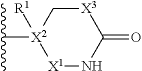

may be on any available carbon or nitrogen atom on Ring E, Ring F, or Ring G, including the carbon atom to which Ring E or Ring G are fused to Ring F.

In some embodiments, a compound of formula I-e above is provided as a compound of formula I-e-1 or formula I-e-2:

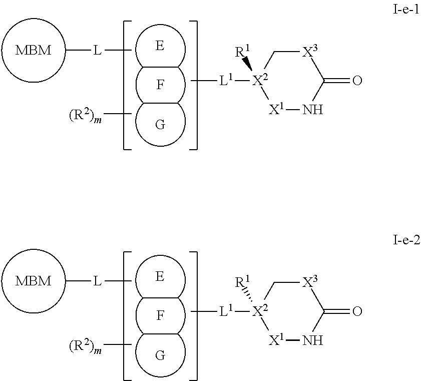

or a pharmaceutically acceptable salt thereof, wherein:
each of MBM, Ring E, Ring F, Ring G, L, L¹, R¹, R², X¹, X², X³, and m is as defined above.

In some embodiments, a compound of formula I-e above is provided as a compound of formula I-e-3:

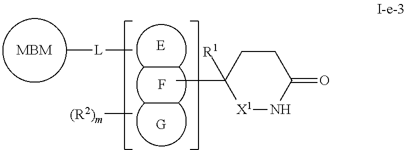

or a pharmaceutically acceptable salt thereof, wherein:
each of MBM, Ring E, Ring F, Ring G, L, R¹, R², X¹, and m is as defined above.

In certain embodiments, the present invention provides a compound of formula I, wherein LBM is an E3 ubiquitin ligase (cereblon) binding moiety thereby forming a compound of formula I-f:

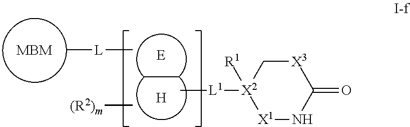

or a pharmaceutically acceptable salt thereof, wherein L and MBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

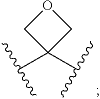 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R² is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R₆ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring E is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
Ring H is a fused ring selected from a 7-9 membered saturated or partially unsaturated carbocyclyl or heterocyclyl ring with 1-3 heteroatoms independently selected from boron, nitrogen, oxygen, silicon, and sulfur, wherein Ring E is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3, or 4.

Where a point of attachment of

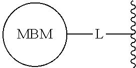

is depicted on Ring E or Ring H, it is intended, and one of ordinary skill in the art would appreciate, that the point of attachment of

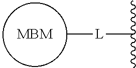

may be on any available carbon or nitrogen atom on Ring E or Ring H including the carbon atom to which Ring E and Ring H are fused.

Where a point of attachment of —(R²)ₘ is depicted on Ring E and Ring H, it is intended, and one of ordinary skill in the art would appreciate, that the point of attachment of —(R²)ₘ may be on any available carbon or nitrogen atom on Ring E or Ring H including the carbon atom to which Ring E and Ring H are fused.

Where a point of attachment of

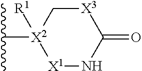

is depicted on Ring E and Ring H, it is intended, and one of ordinary skill in the art would appreciate, that the point of attachment of

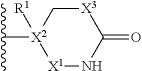

may be on any available carbon or nitrogen atom on Ring E or Ring H including the carbon atom to which Ring E and Ring H are fused.

In some embodiments, a compound of formula I-f above is provided as a compound of formula I-f-1 or formula I-f-2:

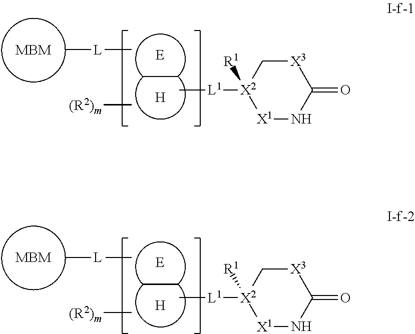

or a pharmaceutically acceptable salt thereof, wherein:
each of MBM, Ring E, Ring H, L, L¹, R¹, R², X¹, X², X³, and m is as defined above.

In some embodiments, a compound of formula I-f above is provided as a compound of formula I-f-3:

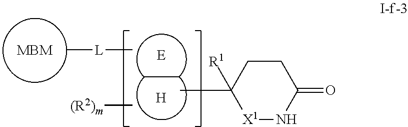

or a pharmaceutically acceptable salt thereof, wherein:
each of MBM, Ring E, Ring H, L, R¹, R², X¹, and m is as defined above.

In certain embodiments, the present invention provides a compound of formula I, wherein LBM is an E3 ubiquitin ligase (cereblon) binding moiety thereby forming a compound of formula I-g:

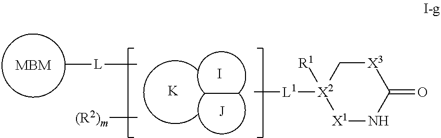

or a pharmaceutically acceptable salt thereof, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

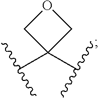 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R² is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring I and J is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
Ring K is a fused ring selected from a 6-12 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—; and
m is 0, 1, 2, 3, or 4.

Where a point of attachment of

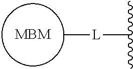

is depicted on Ring I, Ring J, and Ring K, it is intended, and one of ordinary skill in the art would appreciate, that the point of attachment of

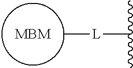

may be on any available carbon or nitrogen atom on Ring I, Ring J, or Ring K, including the carbon atom to which Ring I, Ring J, and Ring K are fused.

Where a point of attachment of —(R²)ₘ is depicted on Ring I, Ring J, and Ring K, it is intended, and one of ordinary skill in the art would appreciate, that the point of attachment of —(R²)ₘ may be on any available carbon or nitrogen atom on Ring I, Ring J, or Ring K, including the carbon atom to which Ring I, Ring J, and Ring K are fused.

Where a point of attachment of

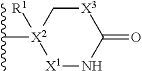

is depicted on Ring I, Ring J, and Ring K, it is intended, and one of ordinary skill in the art would appreciate, that the point of attachment of

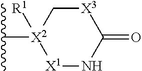

may be on any available carbon or nitrogen atom on Ring I, Ring J, or Ring K, including the carbon atom to which Ring I, Ring J, and Ring K are fused.

In some embodiments, a compound of formula I-g above is provided as a compound of formula I-g-1 or formula I-g-2:

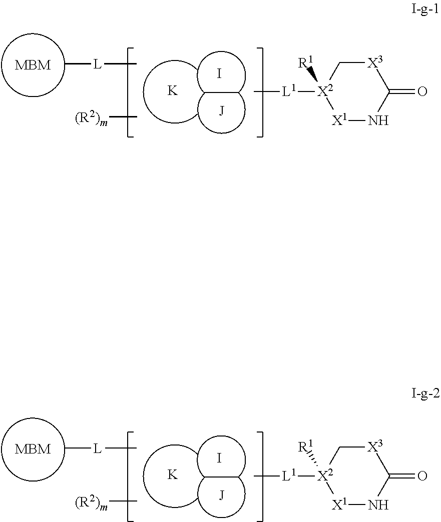

or a pharmaceutically acceptable salt thereof, wherein:
each of MBM, Ring I, Ring J, Ring K, L, L¹, R¹, R², X¹, X², X³, and m is as defined above.

In some embodiments, a compound of formula I-g above is provided as a compound of formula I-g-3:

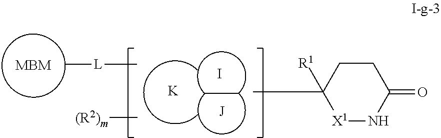

or a pharmaceutically acceptable salt thereof, wherein:
each of MBM, Ring I, Ring J, Ring K, L, R¹, R², X¹, and m is as defined above.

In certain embodiments, the present invention provides a compound of formula I, wherein LBM is an E3 ubiquitin ligase (cereblon) binding moiety thereby forming a compound of formula I-h-1 or I-h-2:

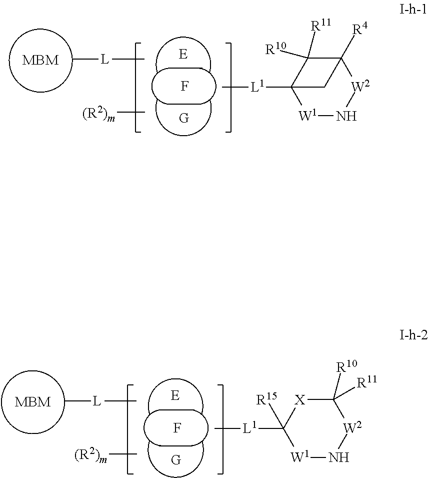

or a pharmaceutically acceptable salt thereof, wherein L and MBM are as defined above and described in embodiments herein, and wherein:
each R² is independently hydrogen, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and
Ring G is independently and optionally further substituted with 1-2 oxo groups; each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3, 4, 5, 6, 7, 8, 9, 10, 11, 12, 13, 14, 15, or 16; and
R⁴, R¹⁰, R¹¹, R¹⁵, W¹, W², and X is as defined in WO 2019/099868, the entirety of each of which is herein incorporated by reference.

Where a point of attachment of

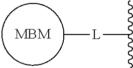

is depicted on Ring E, Ring F, or Ring G, it is intended, and one of ordinary skill in the art would appreciate, that the point of attachment of

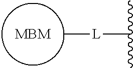

may be on any available carbon or nitrogen atom on Ring E, Ring F, or Ring G, including the ring to which Ring E or Ring G are fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G are fused to Ring F.

Where a point of attachment of

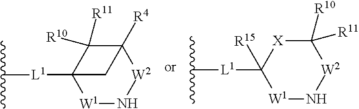

is depicted on Ring E, Ring F, or Ring G, it is intended, and one of ordinary skill in the art would appreciate, that the point of attachment of

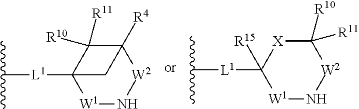

may be on any available carbon or nitrogen atom on Ring E, Ring F, or Ring G, including the carbon atom to which Ring E or Ring G are fused to Ring F.

As described above, in another aspect, the present invention provides a compound of formula I, wherein said compound is a compound of formula I-h-3:

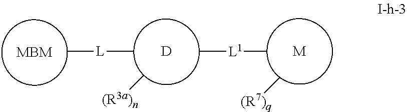

or a pharmaceutically acceptable salt thereof, wherein:
Ring M is selected from

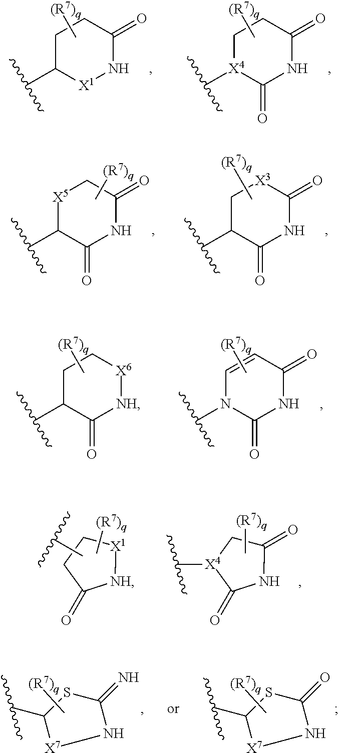

each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

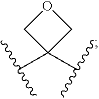 [SMILES: *C1(*)COC1]

each of X³ and X⁵ is independently a bivalent moiety selected from a covalent bond, —CR₂—, —NR—, —O—, —S—, or —SiR₂—;
X⁴ is a trivalent moiety selected from

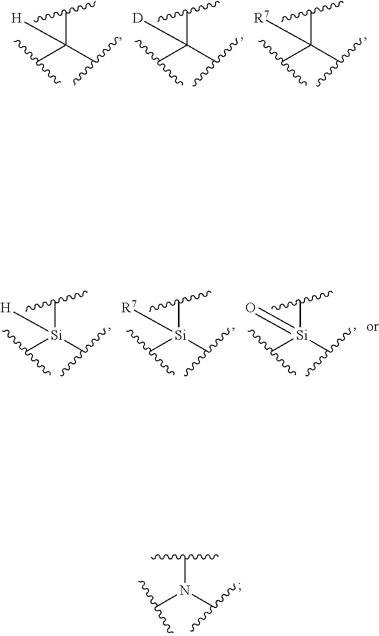

each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{3a} is independently hydrogen, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R₇ is independently hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic; or
   R₇ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur;
   two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur; or
   two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CF(R)—, —C(F)₂—, —N(R)—, —S—, —S(O)₂— or —(C)═CH—;
n is 0, 1, 2, 3, or 4; and
q is 0, 1, 2, 3, or 4.

In certain embodiments, the present invention provides a compound of formula I as a compound of formula I-aa-10:

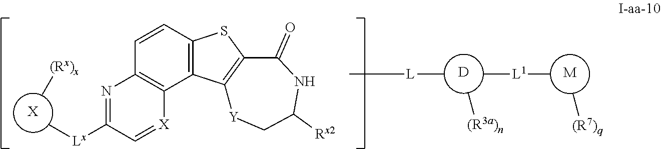

or a pharmaceutically acceptable salt, wherein:
Ring M is selected from

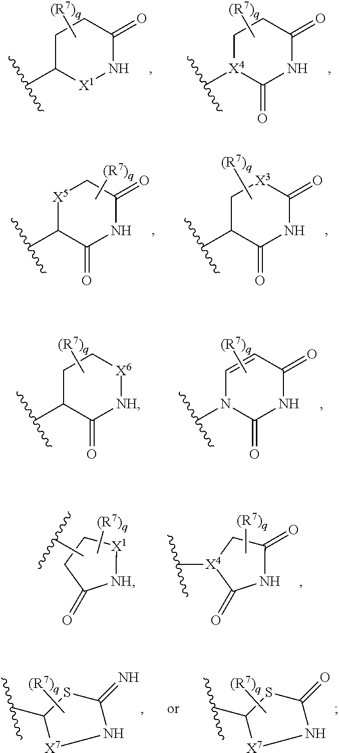

each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

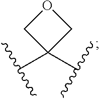 [SMILES: *C1(*)COC1]

each of X³ and X⁵ is independently a bivalent moiety selected from a covalent bond, —CR₂—, —NR—, —O—, —S—, or —SiR₂—;
X⁴ is a trivalent moiety selected from

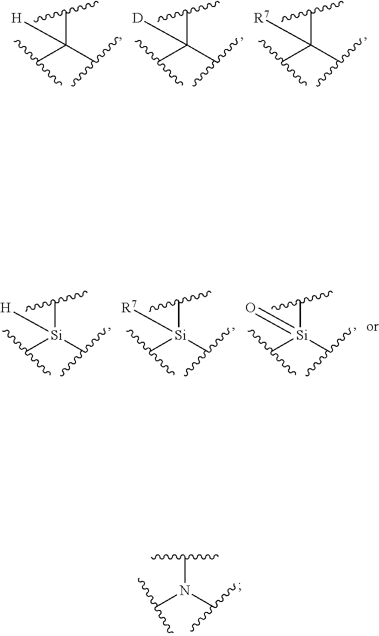

each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{3a} is independently hydrogen, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R₇ is independently hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic; or
   R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur;
   two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur; or
   two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CF(R)—, —C(F)₂—, —N(R)—, —S—, —S(O)₂— or —(C)═CH—;
n is 0, 1, 2, 3, or 4;
q is 0, 1, 2, 3, or 4;
L¹ is a covalent bond or a bivalent, saturated or partially unsaturated, straight or branched C₁₋₅₀ hydrocarbon chain, wherein 0-10 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

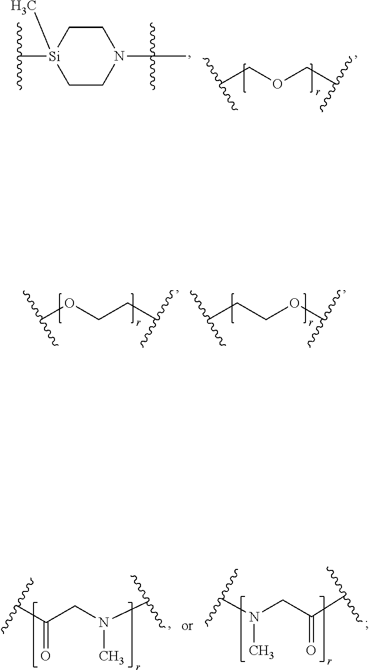

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
Ring X is an optionally substituted ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
X is an optionally substituted carbon or nitrogen atom;
Y is —O—, —S—, or —N(R^{x1})—;
R^{x} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)N(R)₂, —C(O)NROR, —CR₂NRC(O)R, —CR₂NRC(O)N(R)₂, —OC(O)R, —OC(O)N(R)₂, —OP(O)R₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} is a covalent bond or a bivalent, saturated or partially unsaturated, straight or branched C₁₋₅ hydrocarbon chain, wherein 0-3 methylene units of L are independently replaced by —O—, —NR—, —CRF—, —CF₂—, —C(O)—, —S—, —S(O)—, or —S(O)₂—;
R^{x1} and R^{x2} are, independently, hydrogen or an optionally substituted C₁₋₆ aliphatic; and
x is 0, 1, 2, 3, or 4.

In some embodiments, the present invention provides a compound of formula I-aa-10, wherein Y is —N(R^{x1})—, X is a carbon atom, Ring M is

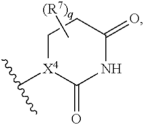

and q is 0 to provide a compound of formula I-aa-11:

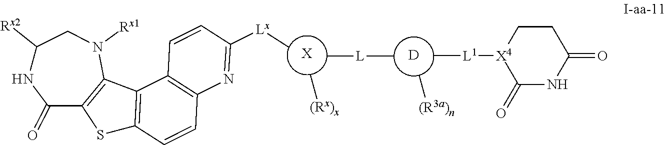

or a pharmaceutically acceptable salt thereof, wherein each of Ring D, Ring X, L, L^{x}, X⁴, R^{x}, R^{x1}, R^{x2}, R^{3a}, x, and n is as defined above and described in embodiments herein, both singly and in combination.

In some embodiments, the present invention provide a compound of formula I-aa-11, wherein Ring X is piperidinylenyl, piperazinylenyl, phenylenyl, or pyridinylenyl.

In some embodiments, the present invention provides a compound of formula I-aa-10,

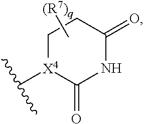

wherein Y is —N(R^{x1})—, X is a carbon atom, Ring M is, q is 0, L^{x} is a bond, and Ring X is piperidinylenyl to provide a compound of formula I-aa-12:

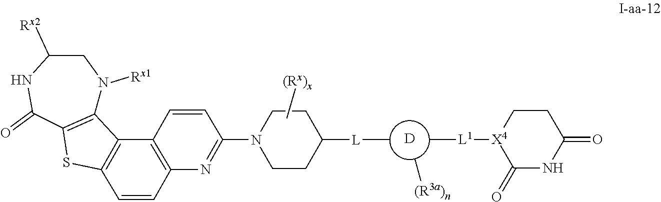

or a pharmaceutically acceptable salt, wherein each of Ring D, L, L¹, X⁴, R^{x}, R^{x}l, R^{x2}, R^{3a}, x, and n is as defined above and described in embodiments herein, both singly and in combination.

In some embodiments, the present invention provides a compound of formula I-aa-10, wherein Y is —N(R^{x1})—, X is a carbon atom, Ring M is

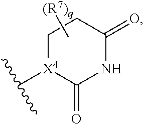

q is 0, L^{x} is a bond, and Ring X is piperazinylenyl to provide a compound of formula I-aa-13:

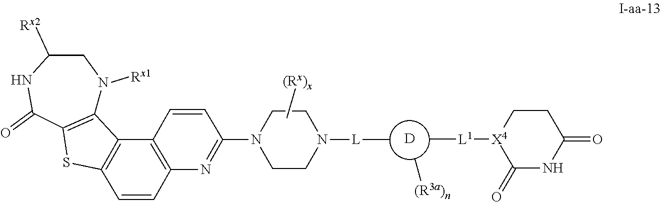

or a pharmaceutically acceptable salt, wherein each of Ring D, L, L¹, X⁴, R^{x}, R^{x}l, R^{x2}, R^{3a}, x, and n is as defined above and described in embodiments herein, both singly and in combination.

In some embodiments, the present invention provides a compound of formula I-aa-10, wherein Y is —N(R^{x1})—, X is a carbon atom, Ring M is

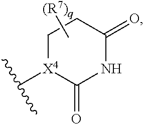

q is 0, L^{x} is a bond, and Ring X is para-fused phenylenyl to provide a compound of formula I-aa-14:

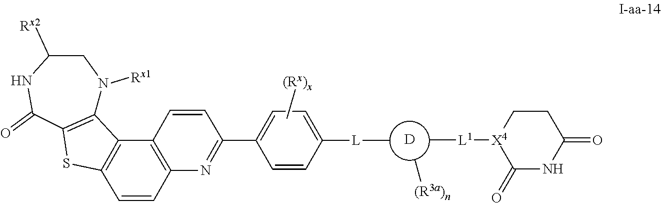

or a pharmaceutically acceptable salt, wherein each of Ring D, L, L¹, X⁴, R^{x}, R^{x1}, R^{x2}, R^{3a}, x, and n is as defined above and described in embodiments herein, both singly and in combination.

In some embodiments, the present invention provides a compound of formula I-aa-10, wherein Y is —N(R^{x1})—, X is a carbon atom, Ring M is

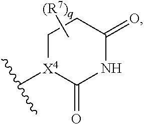

q is 0, L^{x} is a bond, and Ring X is meta-fused phenylenyl to provide a compound of formula I-aa-15:

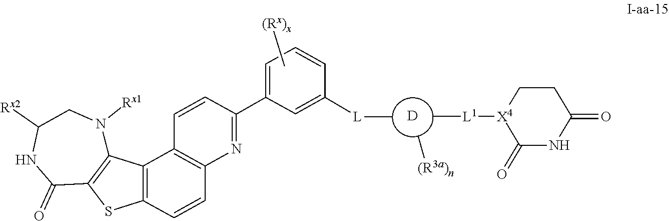

or a pharmaceutically acceptable salt, wherein each of Ring D, L, L¹, X⁴, R^{x}, R^{x1}, R^{x2}, R^{3a}, x, and n is as defined above and described in embodiments herein, both singly and in combination.

In some embodiments, the present invention provides a compound of formula I-aa-10, wherein Y is —N(R^{x1})—, X is a carbon atom, Ring M is

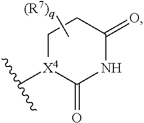

q is 0, L^{x} is a bond, and Ring X is para-fused pyridinylenyl to provide a compound of formula I-aa-16:

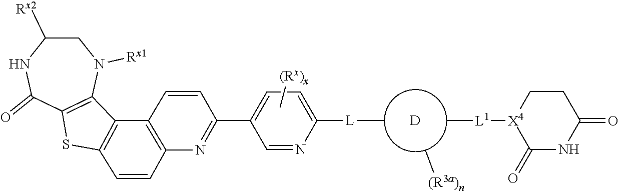

or a pharmaceutically acceptable salt, wherein each of Ring D, L, L¹, X⁴, R^{x}, R^{x1}, R^{x2}, R^{3a}, x, and n is as defined above and described in embodiments herein, both singly and in combination.

In certain embodiments, the present invention provides a compound of formula I as a compound of formula I-bb-5:

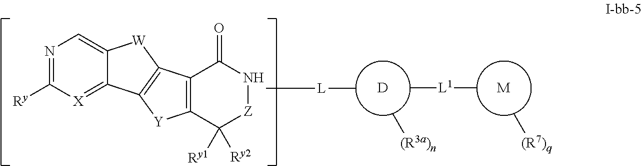

or a pharmaceutically acceptable salt thereof, wherein:
Ring M is selected from

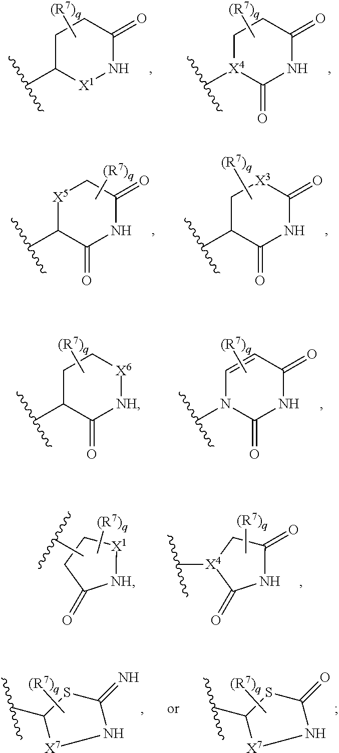

each of X¹, X⁶, and X⁷ is independently a bivalent moiety selected from a covalent bond, —CH₂—, —CHCF₃-, —SO₂—, —S(O)—, —P(O)R—, —P(O)OR—, —P(O)NR₂—, —C(O)—, —C(S)—, or

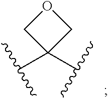 [SMILES: *C1(*)COC1]

each of X³ and X⁵ is independently a bivalent moiety selected from a covalent bond, —CR₂—, —NR—, —O—, —S—, or —SiR₂—;
X⁴ is a trivalent moiety selected from

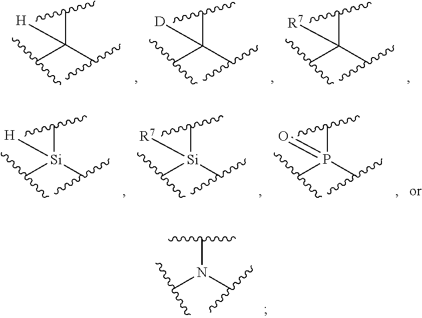

each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{3a} is independently hydrogen, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁷ is independently hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH)₂R, —SiR₃, or an optionally substituted C₁_4 aliphatic; or
   R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur;
   two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur; or
   two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CF(R)—, —C(F)₂—, —N(R)—, —S—, —S(O)₂— or —(C)═CH—;
n is 0, 1, 2, 3, or 4;
q is 0, 1, 2, 3, or 4;
L is a covalent bond or a bivalent, saturated or partially unsaturated, straight or branched C₁₋₅₀ hydrocarbon chain, wherein 0-10 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

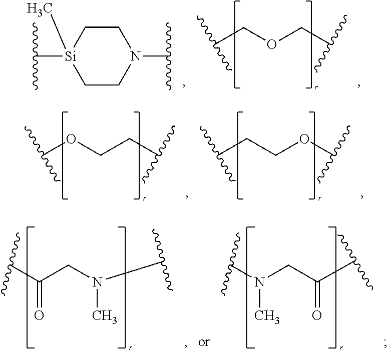

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
R^{y} is hydrogen, halogen, —Y¹—R, an optionally substituted C₁₋₆ aliphatic,

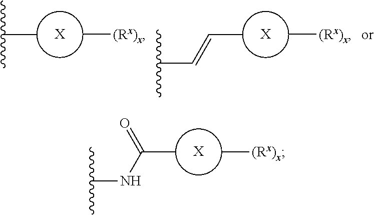

Ring X is an optionally substituted ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{x} is hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)N(R)₂, —C(O)NROR, —CR₂NRC(O)R, —CR₂NRC(O)N(R)₂, —OC(O)R, —OC(O)N(R)₂, —OP(O)R₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
X is an optionally substituted carbon or nitrogen atom;
Y and Y¹ are, independently, a bivalent group selected from —O—, —S— and —NR—;
W is a bivalent group selected from —CH₂—, —CH₂CH₂—, —CH₂CH₂CH₂— and —CH═CH—;
Z is a bivalent group selected from a bond and —C(R^{y3})(R^{y4})—;
R^{y1} and R^{y2} are, independently, hydrogen or taken together with their intervening atoms to form an optionally substituted 4-7 membered saturated or partially unsaturated carbocyclic or heterocyclic ring having 1-3 heteroatoms independently selected from nitrogen, oxygen, and sulfur;
R^{y3} and R^{y4} are, independently, hydrogen or taken together with their intervening atoms to form an optionally substituted 4-7 membered saturated or partially unsaturated carbocyclic or heterocyclic ring having 1-3 heteroatoms independently selected from nitrogen, oxygen, and sulfur; and
x is 0, 1, 2, 3, or 4.

In some embodiments, the present invention provides a compound of formula I-bb-5, wherein Ring M is

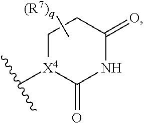

### q is 0, and R^{y} is

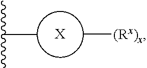

to provide a compound of formula I-bb-6:

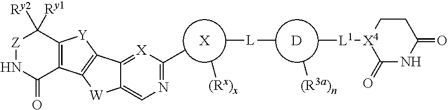

or a pharmaceutically acceptable salt thereof, wherein each of Ring D, Ring X, L, L¹, W, X, Y, Z, R^{x}, R^{y1}, R^{y2}, R^{3a}, n and x is as defined above and described in embodiments herein, both singly and in combination.

In certain embodiments, the present invention provides a compound of formula I as a compound of formula I-cc-10:

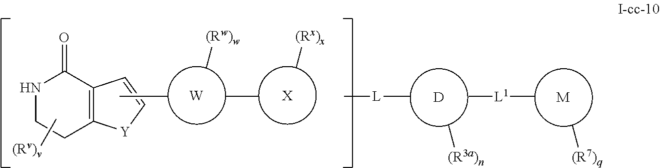

or a pharmaceutically acceptable salt thereof, wherein:
Ring M is selected from

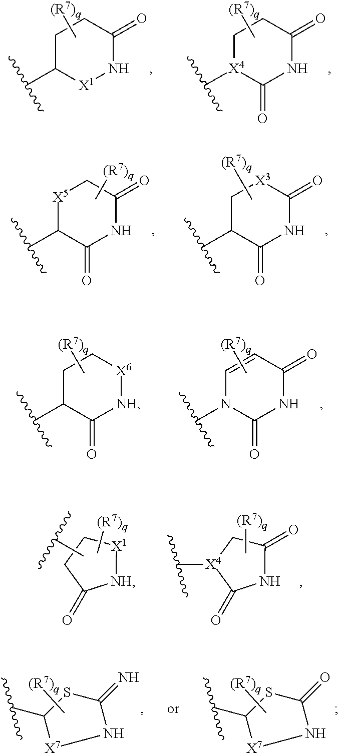

each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

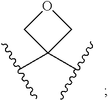 [SMILES: *C1(*)COC1]

each of X³ and X⁵ is independently a bivalent moiety selected from a covalent bond, —CR₂—, —NR—, —O—, —S—, or —SiR₂—;
X⁴ is a trivalent moiety selected from

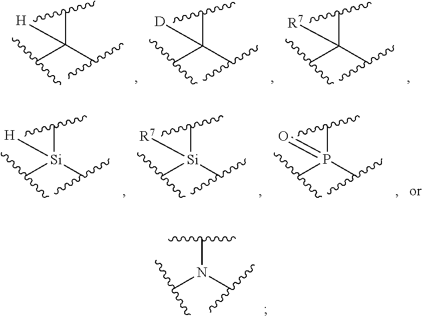

each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R^{3a} is independently hydrogen, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁷ is independently hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic; or
   R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur;
   two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur; or
   two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CF(R)—, —C(F)₂—, —N(R)—, —S—, —S(O)₂— or —(C)═CH—;
n is 0, 1, 2, 3, or 4;
q is 0, 1, 2, 3, or 4;
L¹ is a covalent bond or a bivalent, saturated or partially unsaturated, straight or branched C₁₋₅₀ hydrocarbon chain, wherein 0-10 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

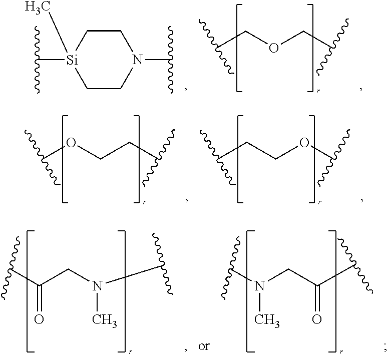

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
Y is —O—, —S— or —NR—;
Ring W is a bivalent ring selected from phenylenyl, naphthylenyl, a 5-10 membered heteroarylenyl containing 1-4 heteroatoms independently selected from nitrogen, oxygen, and sulfur, and a 5-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur;
Ring X is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{v}, R^{w}, and R^{x} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R; or:
   two R^{v} groups on the same or different atoms are optionally taken together with their intervening atoms to form an optionally substituted 3-6 membered saturated or partially unsaturated carbocyclic ring or heterocyclic ring with 1-2 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-9 membered heteroaryl ring having 1-4 heteroatoms independently selected from nitrogen, oxygen, and sulfur;
v is 0, 1, 2, 3, or 4;
w is 0, 1, 2, 3, or 4; and
x is 0, 1, 2, 3, or 4.

In some embodiments, the present invention provides a compound of formula I-cc-10, wherein Y is —NH—, Ring M is

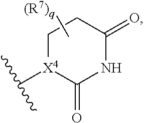

and q is 0 to provide a compound of formula I-cc-11:

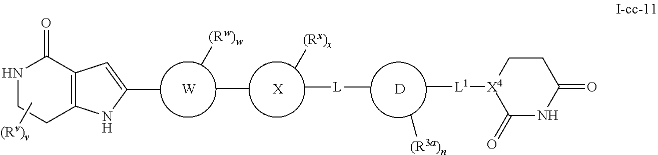

or a pharmaceutically acceptable salt thereof, wherein each of Ring D, Ring, W, Ring X, L, L, X⁴, R^{v}, R^{w}, R^{x}, R^{3a}, v, w, x, and n is as defined above and described in embodiments herein, both singly and in combination.

In some embodiments, the present invention provide a compound of formula I-cc-11, wherein Ring W is a meta-fused phenylenyl, pridinylenyl, or primidinylenyl.

In some embodiments, the present invention provides a compound of formula I-cc-10, wherein Y is —NH—, Ring W is a meta-fused pridinylenyl, Ring M is

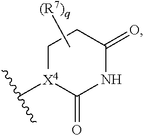

and q is 0 to provide a compound of formula I-cc-12:

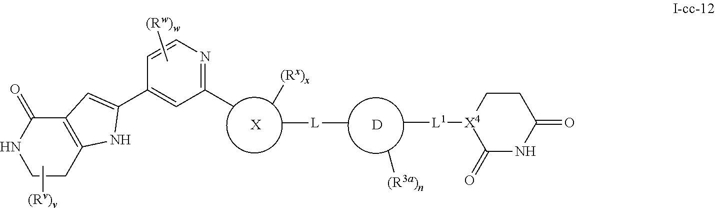

or a pharmaceutically acceptable salt thereof, wherein each of Ring D, Ring X, L, L¹, X⁴, R^{v}, R^{w}, R^{x}, R^{3a}, v, w, x, and n is as defined above and described in embodiments herein, both singly and in combination.

In some embodiments, the present invention provides a compound of formula I-cc-10, wherein Y is —NH—, Ring W is a meta-fused primidinylenyl, Ring M is

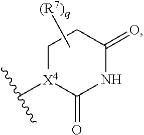

and q is 0 to provide a compound of formula I-cc-13:

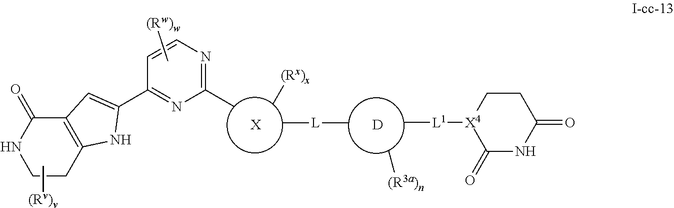

or a pharmaceutically acceptable salt thereof, wherein each of Ring D, Ring X, L, L1, X⁴, R^{v}, R^{w}, R^{x}, R^{3a}, v, w, x, and n is as defined above and described in embodiments herein, both singly and in combination.

In some embodiments, the present invention provides a compound of formula I-cc-10, wherein Y is —NH—, Ring W is a meta-fused pridinylenyl, Ring X is para-fused phenylenyl, Ring M is

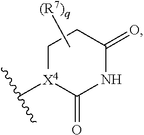

and q is 0 to provide a compound of formula I-cc-14:

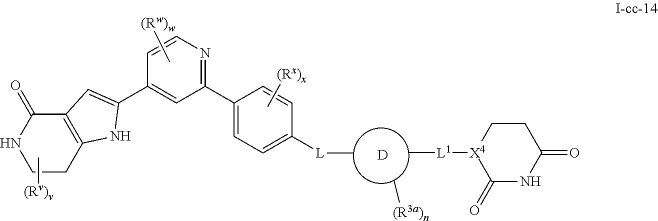

or a pharmaceutically acceptable salt thereof, wherein each of Ring D, L, L¹, X⁴, R^{v}, R^{w}, R^{x}, R^{3a}, v, w, x, and n is as defined above and described in embodiments herein, both singly and in combination.

In some embodiments, the present invention provides a compound of formula I-cc-10, wherein Y is —NH—, Ring W is a meta-fused primidinylenyl, Ring X is para-fused phenylenyl, Ring M is

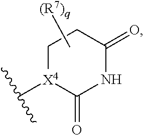

and q is 0 to provide a compound of formula I-cc-15:

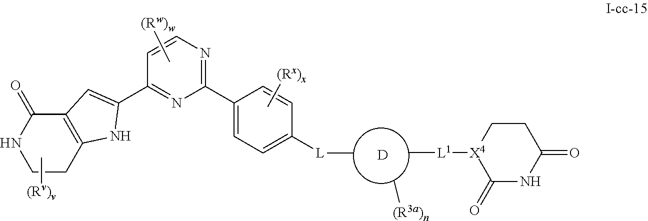

or a pharmaceutically acceptable salt thereof, wherein each of Ring D, L, L¹, X⁴, R^{v}, R^{w}, R^{x}, R^{3a}, v, w, x, and n is as defined above and described in embodiments herein, both singly and in combination.

As defined above and described herein, each of X¹, X⁶, and X⁷ is independently a bivalent moiety selected from a covalent bond, —CH₂—, —C(R)₂—, —C(O)—, —C(S)—, —CH(R)—, —CH(CF₃)—, —P(O)(OR)—, —P(O)(R)—, —P(O)(NR₂)—, —S(O)—, —S(O)₂—, or

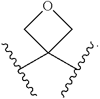 [SMILES: *C1(*)COC1]

In some embodiments, X¹, X⁶, and/or X⁷ is a covalent bond. In some embodiments, X¹, X⁶, and/or X⁷ is —CH₂—. In some embodiments, X¹, X⁶, and/or X⁷ is —CR₂—. In some embodiments, X¹, X⁶, and/or X⁷ is —C(O)—. In some embodiments, X¹, X⁶, and/or X⁷ is —C(S)—. In some embodiments, X¹, X⁶, and/or X⁷ is —CH(R)—. In some embodiments, X¹, X⁶, and/or X⁷ is —CH(CF₃)—. In some embodiments, X¹, X⁶, and/or X⁷ is —P(O)(OR)—. In some embodiments, X¹, X⁶, and/or X⁷ is —P(O)(R)—. In some embodiments, X¹, X⁶, and/or X⁷ is —P(O)NR₂—. In some embodiments, X¹, X⁶, and/or X⁷ is —S(O)—. In some embodiments, X¹, X⁶, and/or X⁷ is —S(O)₂—. In some embodiments, X¹, X⁶, and/or X⁷ is

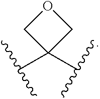 [SMILES: *C1(*)COC1]

In some embodiments, each of X¹, X⁶, and X⁷ are independently selected from those depicted in Table 1 below.

As defined above and described herein, X² is a carbon atom or silicon atom.

In some embodiments, X² is a carbon atom. In some embodiments, X² is a silicon atom.

In some embodiments, X² is selected from those depicted in Table 1, below.

As defined above and described herein, each of X³ and X⁵ is independently a bivalent moiety selected from —CH₂—, —CR₂—, —NR—, —CF₂—, —CHF—, —S—, —CH(R)—, —SiR₂—, or —O—.

In some embodiments, X³ and/or X⁵ is —CH₂—. In some embodiments, X³ and/or X⁵ is —CR₂—. In some embodiments, X³ and/or X⁵ is —NR—. In some embodiments, X³ and/or X⁵ is —CF₂—. In some embodiments, X³ and/or X⁵ is —CHF—. In some embodiments, X³ and/or X⁵ is —S—. In some embodiments, X³ and/or X⁵ is —CH(R)—. In some embodiments, X³ and/or X⁵ is —SiR₂—. In some embodiments, X³ and/or X⁵ is —O—.

In some embodiments, each of X³ and X⁵ is independently selected from those depicted in Table 1 below.

As defined above and described herein, X⁴ is a trivalent moiety selected from

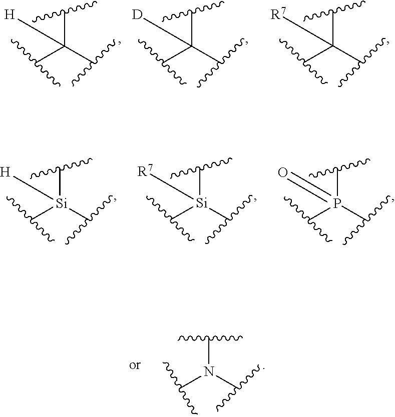

In some embodiments, X⁴ is

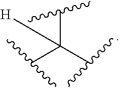 [SMILES: *C(*)*]

In some embodiments, X⁴ is

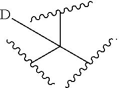 [SMILES: *C(*)(*)[2H]]

In some embodiments, X⁴ is

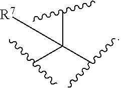

In some embodiments, X⁴ is

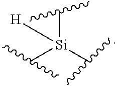 [SMILES: *[SiH](*)*]

In some embodiments, X⁴ is

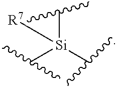

In some embodiments, X⁴ is

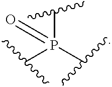 [SMILES: *P(*)(*)=O]

In some embodiments, X⁴ is

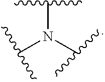 [SMILES: *N(*)*]

In some embodiments, X⁴ is selected from those depicted in Table 1 below.

As defined above and described herein, R¹ is hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, an optionally substituted C₁₋₄ aliphatic, or R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, and sulfur.

In some embodiments, R¹ is hydrogen. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is —P(O)(OR)₂. In some embodiments, R¹ is —P(O)(NR₂)OR. In some embodiments, R¹ is —P(O)(NR₂)₂. In some embodiments, R¹ is —Si(OH)₂R. In some embodiments, R¹ is —Si(OH)(R)₂. In some embodiments, R¹ is —Si(R)₃. In some embodiments, R¹ is an optionally substituted C₁₋₄ aliphatic. In some embodiments, R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, and sulfur.

In some embodiments, R¹ is selected from those depicted in Table 1, below.

As defined above and described herein, each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur.

In some embodiments, R is hydrogen. In some embodiments, R is optionally substituted C₁₋₆ aliphatic. In some embodiments, R is optionally substituted phenyl. In some embodiments, R is optionally substituted 3-7 membered saturated or partially unsaturated heterocyclic ring having 1-2 heteroatoms independently selected from nitrogen, oxygen, and sulfur. In some embodiments, R is optionally substituted 5-6 membered heteroaryl ring having 1-4 heteroatoms independently selected from nitrogen, oxygen, and sulfur. In some embodiments, two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur.

In some embodiments, Ring R is selected from those depicted in Table 1, below.

As defined above and described herein, each of R² and R^{3a} is independently hydrogen, —R⁶, halogen, —CN, —NO₂, —OR, —Si(OH)₂R, —Si(OH)R₂, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R.

In some embodiments, R² and/or R^{3a} is hydrogen. In some embodiments, R² and/or R^{3a} is —R. In some embodiments, R² and/or R^{3a} is halogen. In some embodiments, R² and/or R^{3a} is —CN. In some embodiments, R² and/or R^{3a} is —NO₂. In some embodiments, R² and/or R^{3a} is —OR. In some embodiments, R² and/or R^{3a} is —Si(OH)₂R. In some embodiments, R² and/or R^{3a} is —Si(OH)R₂. In some embodiments, R² and/or R^{3a} is —SR. In some embodiments, R² and/or R^{3a} is —NR₂. In some embodiments, R² and/or R^{3a} is —SiR₃. In some embodiments, R² and/or R^{3a} is —S(O)₂R. In some embodiments, R² and/or R^{3a} is —S(O)₂NR₂. In some embodiments, R² and/or R^{3a} is —S(O)R. In some embodiments, R² and/or R^{3a} is —C(O)R. In some embodiments, R² and/or R^{3a} is —C(O)OR. In some embodiments, R² and/or R^{3a} is —C(O)NR₂. In some embodiments, R² and/or R^{3a} is —C(O)N(R)OR. In some embodiments, R² and/or R^{3a} is —C(R)₂N(R)C(O)R. In some embodiments, R² and/or R^{3a} is —C(R)₂N(R)C(O)NR₂. In some embodiments, R² and/or R^{3a} is —OC(O)R. In some embodiments, R² and/or R^{3a} is —OC(O)NR₂. In some embodiments, R² and/or R^{3a} is —OP(O)R₂. In some embodiments, R² and/or R^{3a} is —OP(O)(OR)₂. In some embodiments, R² and/or R^{3a} is —OP(O)(OR)NR₂. In some embodiments, R² and/or R^{3a} is —OP(O)(NR₂)₂—. In some embodiments, R² and/or R^{3a} is —N(R)C(O)OR. In some embodiments, R² and R^{3a} is independently —N(R)C(O)R. In some embodiments, R² and/or R^{3a} is —N(R)C(O)NR₂. In some embodiments, R² and/or R^{3a} is —NP(O)R₂. In some embodiments, R² and/or R^{3a} is —N(R)P(O)(OR)₂. In some embodiments, R² and/or R^{3a} is —N(R)P(O)(OR)NR₂. In some embodiments, R² and/or R^{3a} is —N(R)P(O)(NR₂)₂. In some embodiments, R² and/or R^{3a} is —N(R)S(O)₂R.

In some embodiments, R² and R^{3a} is independently —OH. In some embodiments, R² and R^{3a} is independently —NH₂. In some embodiments, R² and R^{3a} is independently —CH₂NH₂. In some embodiments, R² and R^{3a} is independently —CH₂NHCOMe. In some embodiments, R² and R^{3a} is independently —CH₂NHCONHMe. In some embodiments, R² and R^{3a} is independently —NHCOMe. In some embodiments, R² and R^{3a} is independently —NHCONHEt. In some embodiments, R² and R^{3a} is independently —SiMe₃. In some embodiments, R² and R^{3a} is independently —SiMe₂OH. In some embodiments, R² and R^{3a} is independently —SiMe(OH)₂. In some embodiments R² and/or R^{3a} is

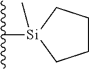 [SMILES: *[Si]1(C)CCCC1]

In some embodiments, R² and/or R^{3a} is Br. In some embodiments, R² and/or R^{3a} is C₁. In some embodiments, R² and/or R^{3a} is F. In some embodiments, R² and/or R^{3a} is Me. In some embodiments, R² and/or R^{3a} is —NHMe. In some embodiments, R² and/or R^{3a} is —NMe₂. In some embodiments, R² and/or R^{3a} is —NHCO₂Et. In some embodiments, R² and/or R^{3a} is —CN. In some embodiments, R² and/or R^{3a} is —CH₂Ph. In some embodiments, R² and/or R^{3a} is —NHCO₂tBu. In some embodiments, R² and/or R^{3a} is —CO₂tBu. In some embodiments, R² and/or R^{3a} is —OMe. In some embodiments, R² and/or R^{3a} is —CF₃.

In some embodiments, R² and R^{3a} are selected from those depicted in Table 1, below.

As defined above and described herein, R³ is hydrogen, halogen, —CN, —NO₂, —OR, —NR₂, —SR, —S(O)₂R, —S(O)₂NR₂, —S(O)R, —C(O)R, —C(O)OR, —C(O)NR₂, —C(O)NR(OR), —OC(O)R, —OC(O)NR₂, —OP(O)(OR)₂, —OP(O)(NR₂)₂, —OP(O)(OR)NR₂, —N(R)C(O)R, —N(R)C(O)OR, —N(R)C(O)NR₂, —N(R)S(O)₂R, —N(R)S(O)₂NR₂, —N(R)P(O)(OR)₂, —N(R)P(O)(OR)NR₂, —P(O)(OR)₂, —P(O)(NR₂)OR, —P(O)(NR₂)₂, —Si(OH)₂R, —Si(OH)(R)₂, or —Si(R)₃.

In some embodiments, R³ is hydrogen. In some embodiments, R³ is halogen. In some embodiments, R³ is —CN. In some embodiments, R³ is —NO₂. In some embodiments, R³ is —OR. In some embodiments, R³ is —NR₂. In some embodiments, R³ is —SR. In some embodiments, R³ is —S(O)₂R. In some embodiments, R³ is —S(O)₂NR₂. In some embodiments, R³ is —S(O)R. In some embodiments, R³ is —C(O)R. In some embodiments, R³ is —C(O)OR. In some embodiments, R³ is —C(O)NR₂. In some embodiments, R³ is —C(O)NR(OR). In some embodiments, R³ is —OC(O)R. In some embodiments, R³ is —OC(O)NR₂. In some embodiments, R³ is —OP(O)(OR)₂. In some embodiments, R³ is —OP(O)(NR₂)₂. In some embodiments, R³ is —OP(O)(OR)NR₂. In some embodiments, R³ is —N(R)C(O)R. In some embodiments, R³ is —N(R)C(O)OR. In some embodiments, R³ is —N(R)C(O)NR₂. In some embodiments, R³ is —N(R)S(O)₂R. In some embodiments, R³ is —N(R)S(O)₂NR₂. In some embodiments, R³ is —N(R)P(O)(OR)₂. In some embodiments, R³ is —N(R)P(O)(OR)NR₂. In some embodiments, R³ is —P(O)(OR)₂. In some embodiments, R³ is —P(O)(NR₂)OR. In some embodiments, R³ is —P(O)(NR₂)₂. In some embodiments, R³ is —Si(OH)₂R. In some embodiments, R³ is —Si(OH)(R)₂. In some embodiments, R³ is —Si(R)₃.

In some embodiments, R³ is methyl. In some embodiments, R³ is —OCH₃. In some embodiments, R³ is chloro.

In some embodiments, R³ is selected from those depicted in Table 1, below.

As defined above and described herein, each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —P(O)(OR)₂, —P(O)(NR₂)OR, or —P(O)(NR₂)₂.

In some embodiments, R⁴ is hydrogen. In some embodiments, R⁴ is —R⁶. In some embodiments, R⁴ is halogen. In some embodiments, R⁴ is —CN. In some embodiments, R⁴ is —NO₂. In some embodiments, R⁴ is —OR. In some embodiments, R⁴ is —SR. In some embodiments, R⁴ is —NR₂. In some embodiments, R⁴ is —S(O)₂R. In some embodiments, R⁴ is —S(O)₂NR₂. In some embodiments, R⁴ is —S(O)R. In some embodiments, R⁴ is —C(O)R. In some embodiments, R⁴ is —C(O)OR. In some embodiments, R⁴ is —C(O)NR₂. In some embodiments, R⁴ is —C(O)N(R)OR. In some embodiments, R⁴ is —OC(O)R. In some embodiments, R⁴ is —OC(O)NR₂. In some embodiments, R⁴ is —N(R)C(O)OR. In some embodiments, R⁴ is —N(R)C(O)R. In some embodiments, R⁴ is —N(R)C(O)NR₂. In some embodiments, R⁴ is —N(R)S(O)₂R. In some embodiments, R⁴ is —P(O)(OR)₂. In some embodiments, R⁴ is —P(O)(NR₂)OR. In some embodiments, R⁴ is —P(O)(NR₂)₂.

In some embodiments, R⁴ is methyl. In some embodiments, R⁴ is ethyl. In some embodiments, R⁴ is cyclopropyl.

In some embodiments, R⁴ is selected from those depicted in Table 1, below.

As defined above and described herein, R⁵ is hydrogen, an optionally substitute C₁₋₄ aliphatic, or —CN.

In some embodiments, R⁵ is hydrogen. In some embodiments, R⁵ is an optionally substituted C₁₋₄ aliphatic. In some embodiments, R⁵ is —CN.

In some embodiments, R⁵ is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur.

In some embodiments, R⁶ is an optionally substituted C₁₋₆ aliphatic. In some embodiments, R⁶ is an optionally substituted phenyl. In some embodiments, R⁶ is an optionally substituted 4-7 membered saturated or partially unsaturated heterocyclic ring having 1-3 heteroatoms independently selected from boron, nitrogen, oxygen, silicon, and sulfur. In some embodiments, R⁶ is an optionally substituted 5-6 membered heteroaryl ring having 1-4 heteroatoms independently selected from boron, nitrogen, oxygen, silicon, and sulfur.

In some embodiments, R⁶ is selected from those depicted in Table 1, below.

As defined generally above, each R¹ is independently hydrogen, halogen, —CN, —OR, —SR, —S(O)R, —S(O)₂R, —N(R)₂, —P(O)(R)₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic, or R¹ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur, or two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur, or two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur.

In some embodiments, R⁷ is hydrogen. In some embodiments, R⁷ is halogen. In some embodiments, R⁷ is —CN. In some embodiments, R⁷ is —OR. In some embodiments, R⁷ is —SR. In some embodiments, R⁷ is —S(O)R. In some embodiments, R⁷ is —S(O)₂R. In some embodiments, R⁷ is —NR₂. In some embodiments, R⁷ is —Si(R)₃. In some embodiments, R⁷ is —P(O)(R)₂. In some embodiments, R⁷ is —P(O)(OR)₂. In some embodiments, R⁷ is —P(O)(NR₂)OR. In some embodiments, R⁷ is —P(O)(NR₂)₂. In some embodiments, R⁷ is —Si(OH)R₂. In some embodiments, R⁷ is —Si(OH)₂R. In some embodiments, R⁷ is an optionally substituted C₁₋₄ aliphatic. In some embodiments, R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur. In some embodiments, two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur. In some embodiments, two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur. In some embodiments, two R⁷ groups on adjacent carbon atoms are optionally taken together with their intervening atoms to form a 7-13 membered saturated, partially unsaturated, bridged heterocyclic ring, or a spiro heterocyclic ring having 1-3 heteroatoms, independently selected from boron, nitrogen, oxygen, silicon, and sulfur.

In some embodiments, R⁷ is selected from hydrogen, halogen, —CN, —OR, —NR₂, or C₁₋₄ alkyl. In some embodiments, R⁷ is selected from hydrogen, halogen, —CN, or C₁₋₄ alkyl. In some embodiments, R⁷ is fluoro. In some embodiments, two R⁷ groups on the same carbon are optionally taken together with their intervening atoms to form a 3- or 4-membered spiro fused ring.

In some embodiments, R⁷ is selected from those depicted in Table 1 below.

As defined above and described herein, Ring A is a bi- or tricyclic ring selected from

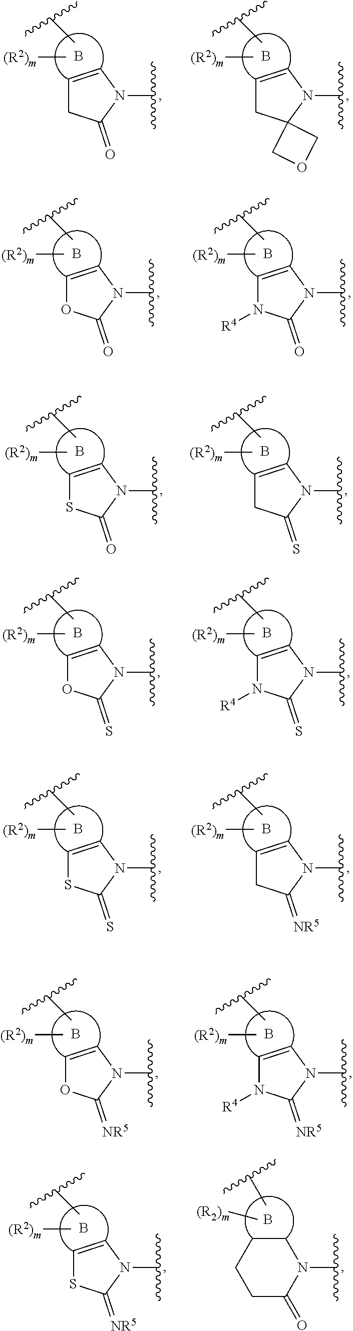

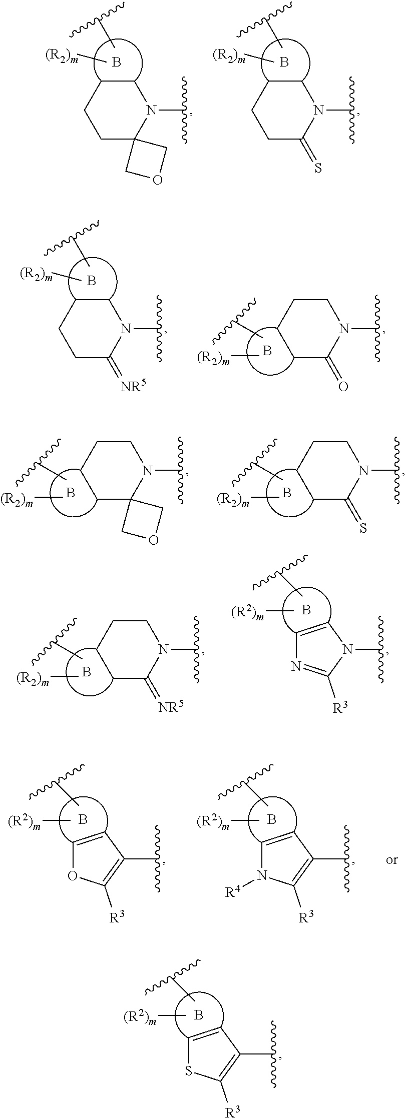

In some embodiments, Ring A is

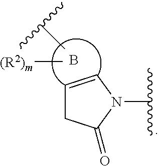

In some embodiments, Ring A is

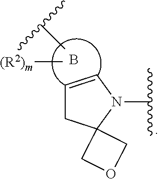

In some embodiments, Ring A is

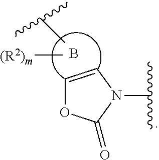

In some embodiments, Ring A is

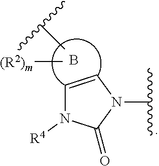

In some embodiments, Ring A is

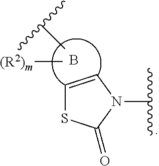

In some embodiments, Ring A is

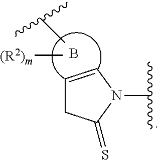

S In some embodiments, Ring A is

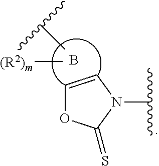

In some embodiments, Ring A is

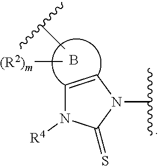

In some embodiments, Ring A is

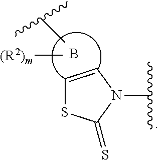

In some embodiments, Ring A is

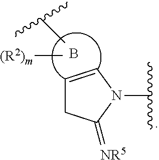

In some embodiments, Ring A is

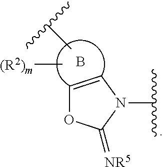

In some embodiments, Ring A is

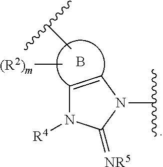

In some embodiments, Ring A is

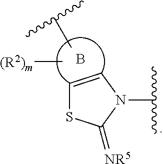

In some embodiments, Ring A is

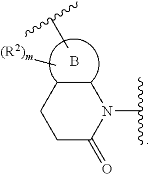

In some embodiments, Ring A is

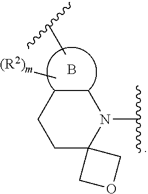

In some embodiments, Ring A is

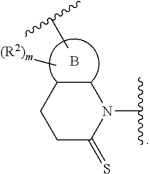

In some embodiments, Ring A is

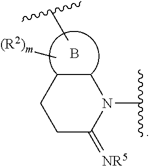

In some embodiments, Ring A is

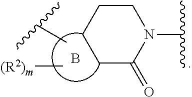

In some embodiments, Ring A is

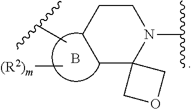

In some embodiments, Ring A is

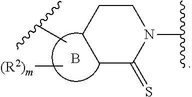

In some embodiments, Ring A is

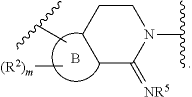

In some embodiments, Ring A is

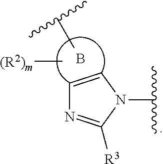

In some embodiments, Ring A is

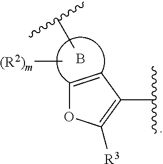

In some embodiments, Ring A is

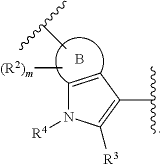

In some embodiments, Ring A is

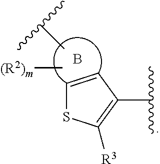

In some embodiments, Ring A is

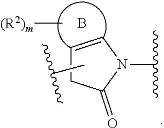

In some embodiments, Ring A is

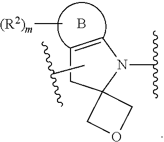

In some embodiments, Ring A is

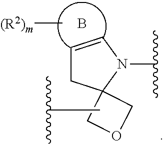

In some embodiments, Ring A is

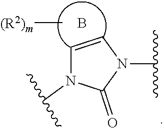

In some embodiments, Ring A is

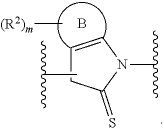

In some embodiments, Ring A is

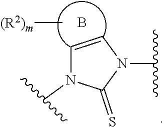

In some embodiments, Ring A is

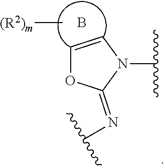

In some embodiments, Ring A is

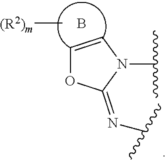

In some embodiments, Ring A is

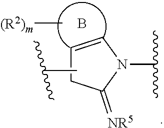

In some embodiments, Ring A is

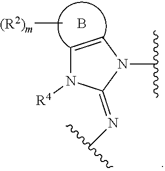

In some embodiments, Ring A is

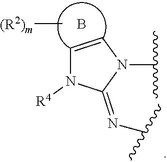

In some embodiments, Ring A is

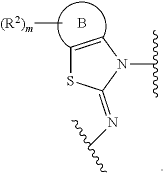

In some embodiments, Ring A is

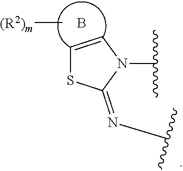

In some embodiments, Ring A is

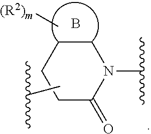

In some embodiments, Ring A is

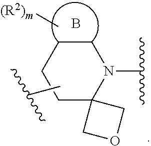

In some embodiments, Ring A is

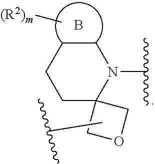

In some embodiments, Ring A is

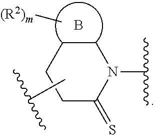

In some embodiments, Ring A is

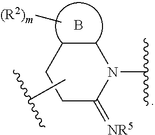

In some embodiments, Ring A is

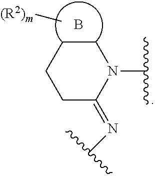

In some embodiments, Ring A is

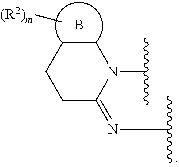

In some embodiments, Ring A is

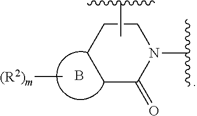

In some embodiments, Ring A is

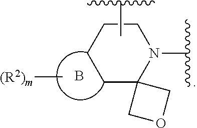

In some embodiments, Ring A is

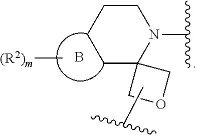

In some embodiments, Ring A is

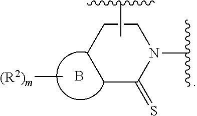

In some embodiments, Ring A is

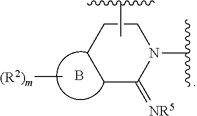

In some embodiments, Ring A is

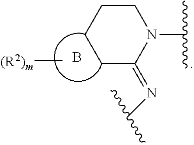

In some embodiments, Ring A is

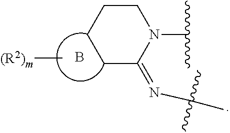

In some embodiments, Ring A is

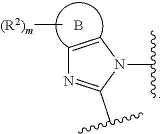

In some embodiments, Ring A is

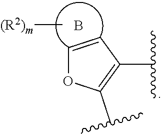

In some embodiments, Ring A is

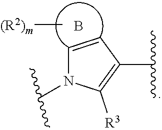

In some embodiments, Ring A is

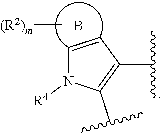

In some embodiments, Ring A is

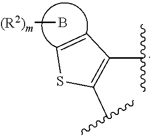

In some embodiments, Ring A is selected from those depicted in Table 1, below.

As defined above and described herein, Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;

In some embodiments, Ring B is a fused 6-membered aryl. In some embodiments, Ring B is a fused 6-membered heteroaryl containing 1-4 heteroatoms independently selected from nitrogen, oxygen, and sulfur. In some embodiments, Ring B is a fused 5 to 7-membered saturated or partially unsaturated carbocyclyl. In some embodiments, Ring B is fused 5 to 7-membered saturated or partially saturated heterocyclyl with 1-3 heteroatoms independently selected from boron, nitrogen, oxygen, silicon, and sulfur. In some embodiments, Ring B is fused 5-membered heteroaryl with 1-4 heteroatoms independently selected from boron, nitrogen, oxygen, silicon, and sulfur.

In some embodiments, Ring B is

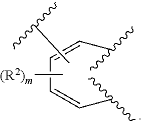

In some embodiments, Ring B is

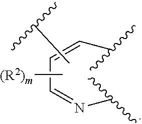

In some embodiments, Ring B is

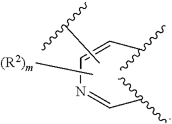

In some embodiments, Ring B is selected from those depicted in Table 1, below.

As defined above and described herein, Ring C is a mono- or bicyclic ring selected from

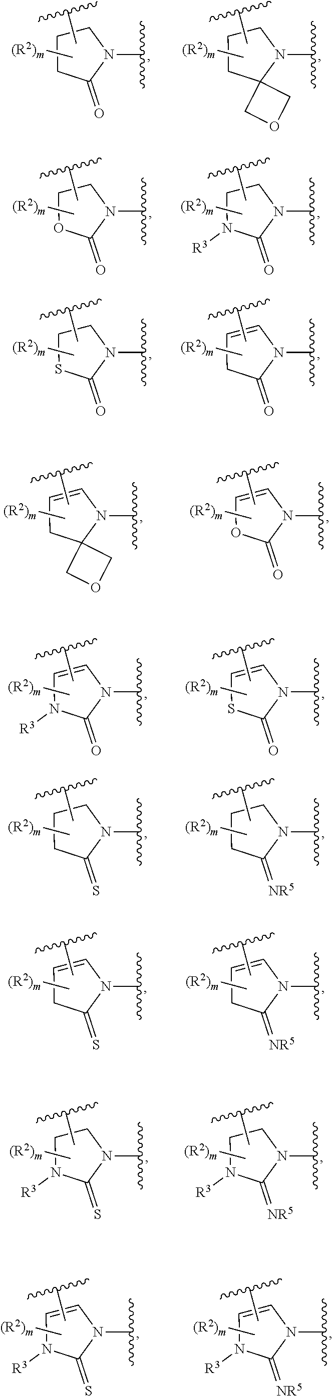

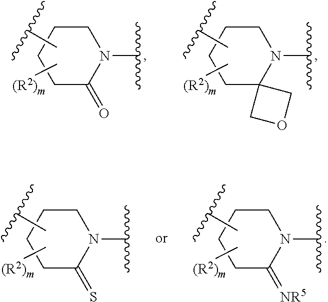

In some embodiments, Ring C is

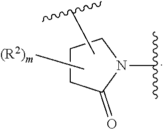

In some embodiments, Ring C is

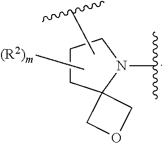

In some embodiments, Ring C is

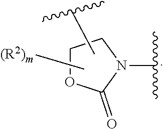

In some embodiments, Ring C is

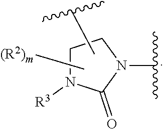

In some embodiments, Ring C is

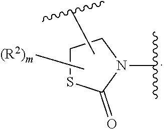

In some embodiments, Ring C is

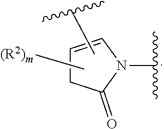

In some embodiments, Ring C is

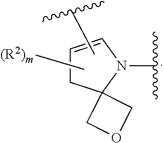

In some embodiments, Ring C is

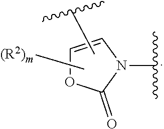

In some embodiments, Ring C is

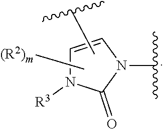

In some embodiments, Ring C is

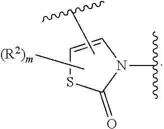

In some embodiments, Ring C is

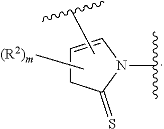

In some embodiments, Ring C is

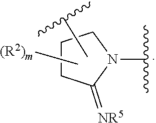

In some embodiments, Ring C is

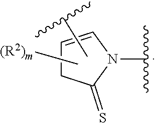

In some embodiments, Ring C is

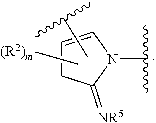

In some embodiments, Ring C is

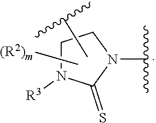

In some embodiments, Ring C is

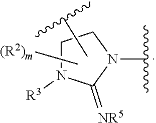

In some embodiments, Ring C is

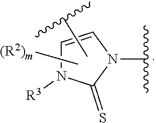

In some embodiments, Ring C is

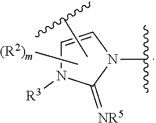

In some embodiments, Ring C is

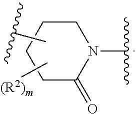

In some embodiments, Ring C is

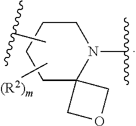

In some embodiments, Ring C is

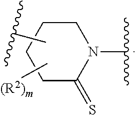

In some embodiments, Ring C is

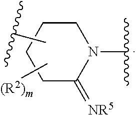

In some embodiments, Ring C is

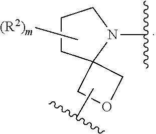

In some embodiments, Ring C is

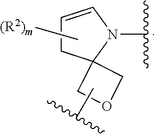

In some embodiments, Ring C is

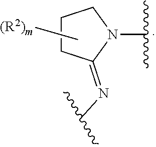

In some embodiments, Ring C is

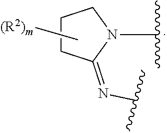

In some embodiments, Ring C is

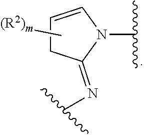

In some embodiments, Ring C is

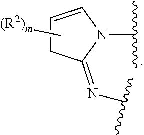

In some embodiments, Ring C is

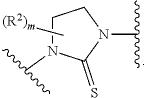

In some embodiments, Ring C is

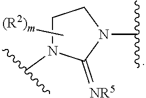

In some embodiments, Ring C is

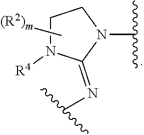

In some embodiments, Ring C is

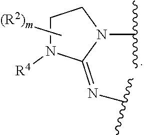

In some embodiments, Ring C is

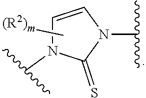

In some embodiments, Ring C is

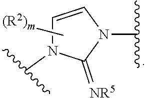

In some embodiments, Ring C is

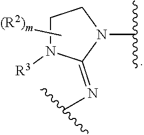

In some embodiments, Ring C is

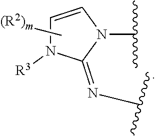

In some embodiments, Ring C is

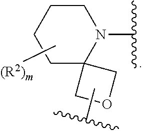

In some embodiments, Ring C is

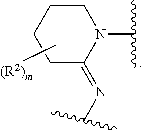

In some embodiments, Ring C is

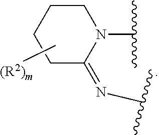

In some embodiments, Ring C is a mono- or bicyclic ring selected from

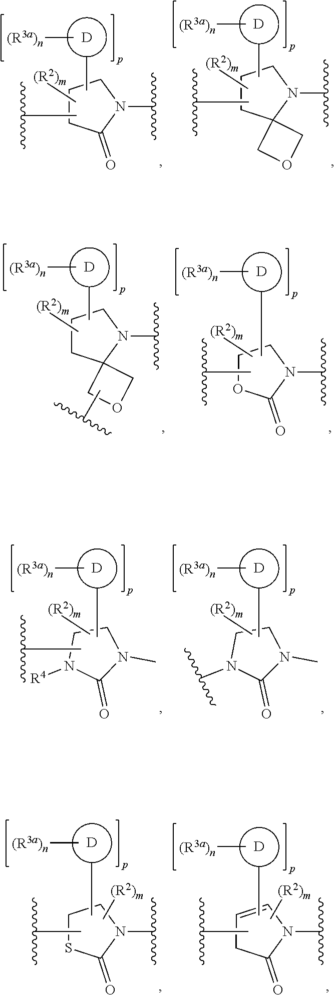

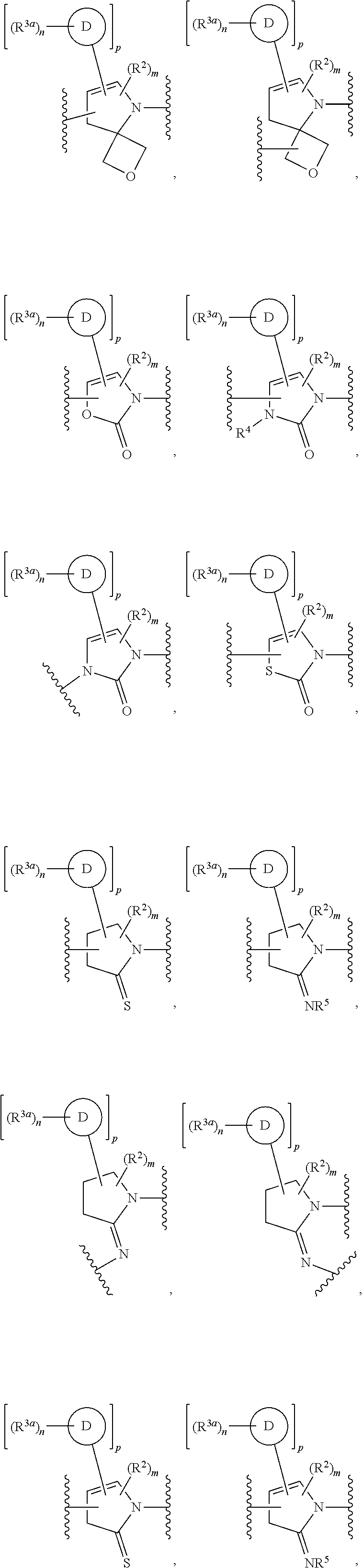

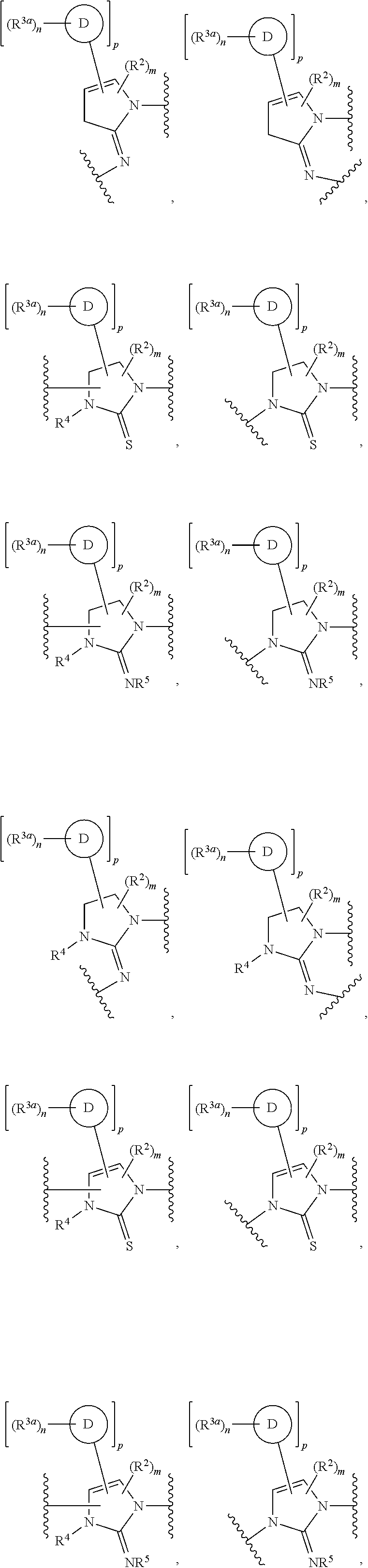

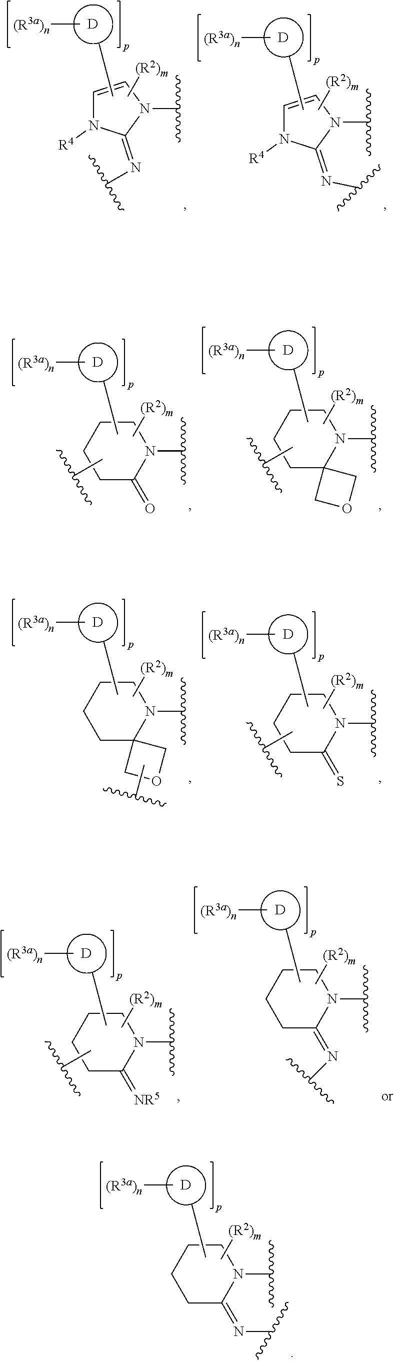

In some embodiments, Ring C is selected from

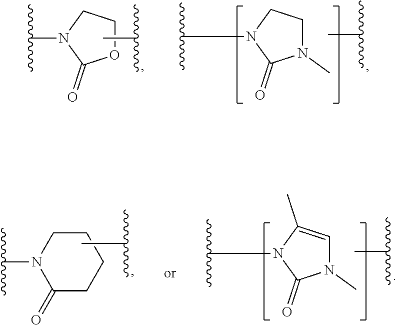

In some embodiments, Ring C is selected from

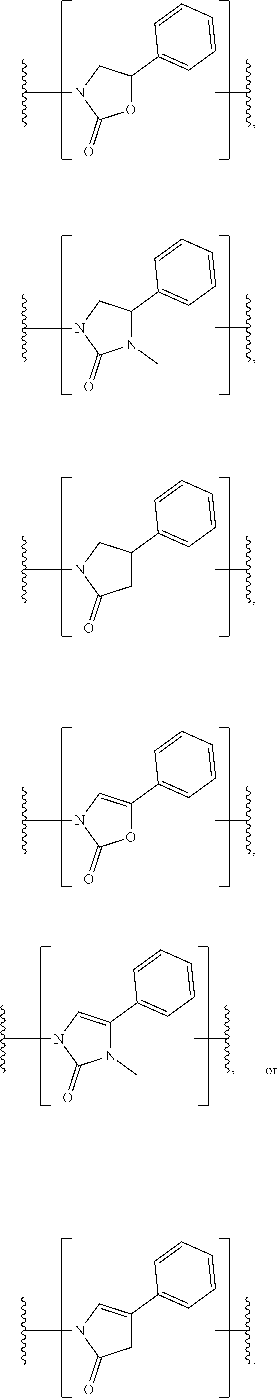

In some embodiments, Ring C is selected from those depicted in Table 1, below.

As defined above and described herein, Ring D is a ring selected from a 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;

In some embodiments, Ring D is a 6 to 10-membered aryl. In some embodiments, Ring D is a 6 to 10-membered heteroaryl containing 1-4 heteroatoms independently selected from nitrogen, oxygen, and sulfur. In some embodiments, Ring D is a 5 to 7-membered saturated or partially unsaturated carbocyclyl. In some embodiments, Ring D is 5 to 7-membered saturated or partially saturated heterocyclyl with 1-3 heteroatoms independently selected from boron, nitrogen, oxygen, silicon, and sulfur. In some embodiments, Ring D is 5-membered heteroaryl with 1-4 heteroatoms independently selected from boron, nitrogen, oxygen, silicon, and sulfur.

In some embodiments, Ring D is phenyl. In some embodiments, Ring D is quinoline. In some embodiments, Ring D is isoquinoline. In some embodiments, Ring D is imidazo[1,2-a]pyridine.

In some embodiments, Ring D is selected from those depicted in Table 1 below.

As defined above and described herein, each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups.

In some embodiments, one or more of Ring E, Ring F, and Ring G is a 6-membered aryl. In some embodiments, one or more of Ring E, Ring F, and Ring G is a 6-membered heteroaryl containing 1-4 heteroatoms independently selected from nitrogen, oxygen, and sulfur. In some embodiments, one or more of Ring E, Ring F, and Ring G is a 5 to 7-membered saturated or partially unsaturated carbocyclyl. In some embodiments, one or more of Ring E, Ring F, and Ring G is a 5 to 7-membered saturated or partially unsaturated heterocyclyl with 1-3 heteroatoms independently selected from boron, nitrogen, oxygen, silicon, and sulfur. In some embodiments, one or more of Ring E, Ring F, and Ring G is a 5-membered heteroaryl with 1-4 heteroatoms independently selected from nitrogen, oxygen and sulfur. In some embodiments, one or more of Ring E, Ring F, and Ring G is and optionally further substituted with 1-2 oxo groups.

In some embodiments, Ring E, Ring F, and Ring G are selected from those depicted in Table 1, below.

As defined above and described herein, Ring H is a ring selected from a 7-9 membered saturated or partially unsaturated carbocyclyl or heterocyclyl ring with 1-3 heteroatoms independently selected from boron, nitrogen, oxygen, silicon, and sulfur, wherein Ring E is optionally further substituted with 1-2 oxo groups.

In some embodiments, Ring H is a ring selected from a 7-9 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups.

In some embodiments, Ring E and Ring H is selected from those depicted in Table 1, below.

As defined above and described herein, each of Ring I and Ring J is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur

In some embodiments, each of Ring I and Ring J is independently a 6-membered aryl. In some embodiments, each of Ring I and Ring J is independently a 6-membered heteroaryl containing 1-4 heteroatoms independently selected from nitrogen, oxygen, and sulfur. In some embodiments, each of Ring I and Ring J is independently a 5 to 7-membered saturated or partially unsaturated carbocyclyl. In some embodiments, each of Ring I and Ring J is independently a 5 to 7-membered saturated or partially unsaturated heterocyclyl ring with 1-3 heteroatoms independently selected from boron, nitrogen, oxygen, silicon, and sulfur. In some embodiments, each of Ring I and Ring J is independently a 5-membered heteroaryl with 1-3 heteroatoms independently selected from nitrogen, oxygen and sulfur.

As defined above and described herein, Ring K is a fused ring selected from a 6-12 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups.

In some embodiments, Ring K is a fused ring selected from a 6-12 membered saturated or partially unsaturated carbocyclyl. In some embodiments, Ring K is a 6-12 membered saturated or partially unsaturated heterocyclyl ring with 1-3 heteroatoms independently selected from boron, nitrogen, oxygen, silicon, and sulfur. In some embodiments, Ring K is optionally further substituted with 1-2 oxo groups.

In some embodiments, Ring I, Ring J, and Ring K is selected from those depicted in Table 1, below.

As defined above and described herein, Ring M is selected from

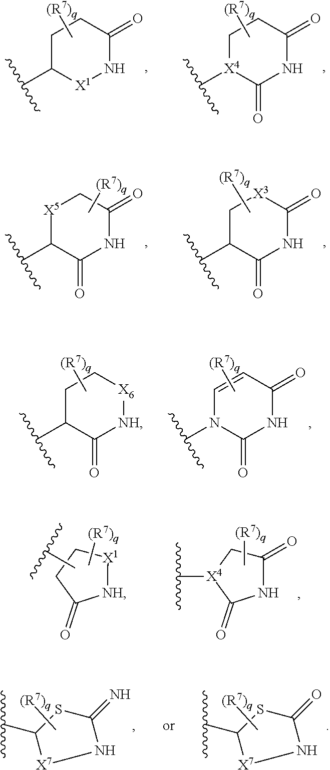

In some embodiments, Ring M is

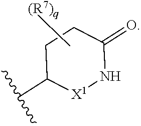

In some embodiments, Ring M is

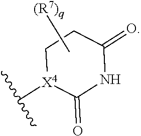

In some embodiments, Ring M is

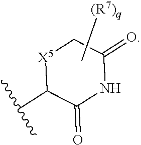

In some embodiments, Ring M is

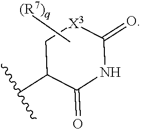

In some embodiments, Ring M is

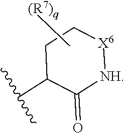

In some embodiments, Ring M is

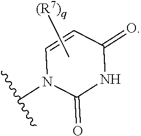

In some embodiments, Ring M is

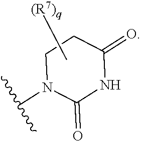

In some embodiments, Ring M is

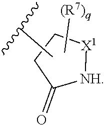

In some embodiments, Ring M is

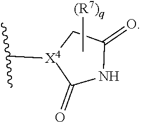

In some embodiments, Ring M is

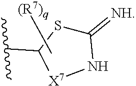

In some embodiments, Ring M is

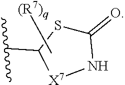

In some embodiments, Ring M is selected from those depicted in Table 1 below.

As defined above and described here, L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;

In some embodiments, L is a covalent bond. In some embodiments, L is a C₁₋₃ aliphatic. In some embodiments, L¹ is —CH₂—. In some embodiments, L is —C(D)(H)—. In some embodiments, L is —C(D)₂-. In some embodiments, L is —CH₂CH₂—. In some embodiments, L is —NR—. In some embodiments, L¹ is —CH₂NR—. In some embodiments, L is or —O—. In some embodiments, L¹ is —CH₂O—. In some embodiments, L¹ is —S—. In some embodiments, L¹ is —OC(O)—. In some embodiments, L¹ is —C(O)O—. In some embodiments, L¹ is —C(O)—. In some embodiments, L¹ is —S(O)—. In some embodiments, L¹ is —S(O)₂—. In some embodiments, L¹ is —NRS(O)₂—. In some embodiments, L is —S(O)₂NR—. In some embodiments, L¹ is —NRC(O)—. In some embodiments, L is —C(O)NR—.

In some embodiments, Ring L¹ is selected from those depicted in Table 1, below.

As defined above and described herein, [symbol] is a single or double bond.

In some embodiments, [symbol] is a single bond. In some embodiments, [symbol] is a double bond.

In some embodiments, [symbol] is selected from those depicted in Table 1, below.

As defined above and described herein, m is 0, 1, 2, 3, 4, 5, 6, 7, 8, 9, 10, 11, 12, 13, 14, 15, or 16.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16.

In some embodiments, m is selected from those depicted in Table 1, below.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In some embodiments, n is selected from those depicted in Table 1, below.

As defined above and described herein, p is 0 or 1.

In some embodiments, p is 0. In some embodiments, p is 1.

In some embodiments, p is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1 below.

In some embodiments, LBM is

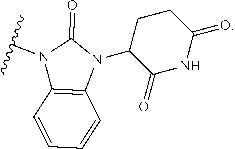 [SMILES: *n1c(=O)n(C2CCC(=O)NC2=O)c2ccccc21]

In some embodiments, LBM is

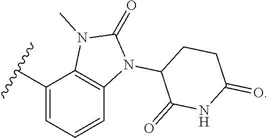 [SMILES: *c1cccc2c1n(C)c(=O)n2C1CCC(=O)NC1=O]

In some embodiments, LBM is

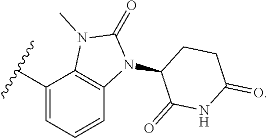 [SMILES: *c1cccc2c1n(C)c(=O)n2[C@H]1CCC(=O)NC1=O]

In some embodiments, LBM is

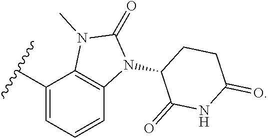 [SMILES: *c1cccc2c1n(C)c(=O)n2[C@@H]1CCC(=O)NC1=O]

In some embodiments, LBM is

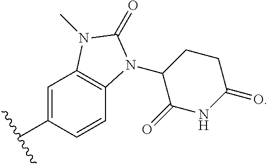 [SMILES: *c1ccc2c(c1)n(C)c(=O)n2C1CCC(=O)NC1=O]

In some embodiments, LBM is

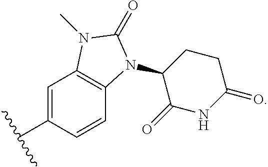 [SMILES: *c1ccc2c(c1)n(C)c(=O)n2[C@H]1CCC(=O)NC1=O]

In some embodiments, LBM is

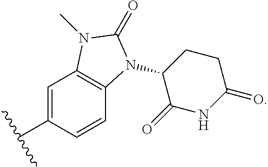 [SMILES: *c1ccc2c(c1)n(C)c(=O)n2[C@@H]1CCC(=O)NC1=O]

In some embodiments, LBM is

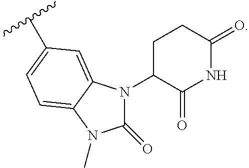 [SMILES: *c1ccc2c(c1)n(C1CCC(=O)NC1=O)c(=O)n2C]

In some embodiments, LBM is

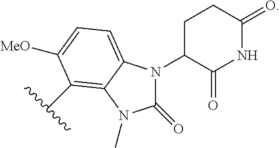 [SMILES: *c1c(OC)ccc2c1n(C)c(=O)n2C1CCC(=O)NC1=O]

In some embodiments, LBM is

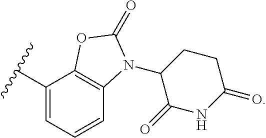 [SMILES: *c1cccc2c1oc(=O)n2C1CCC(=O)NC1=O]

In some embodiments, LBM is

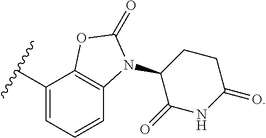 [SMILES: *c1cccc2c1oc(=O)n2[C@H]1CCC(=O)NC1=O]

In some embodiments, LBM is

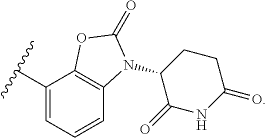 [SMILES: *c1cccc2c1oc(=O)n2[C@@H]1CCC(=O)NC1=O]

In some embodiments, LBM is

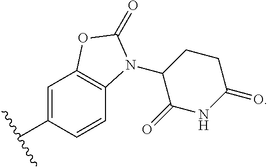 [SMILES: *c1ccc2c(c1)oc(=O)n2C1CCC(=O)NC1=O]

In some embodiments, LBM is

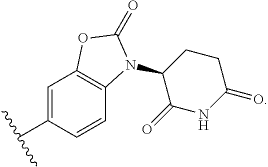 [SMILES: *c1ccc2c(c1)oc(=O)n2[C@H]1CCC(=O)NC1=O]

In some embodiments, LBM is

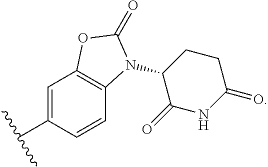 [SMILES: *c1ccc2c(c1)oc(=O)n2[C@@H]1CCC(=O)NC1=O]

In some embodiments, LBM is

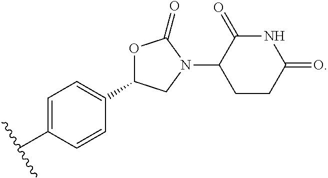 [SMILES: *c1ccc([C@H]2CN(C3CCC(=O)NC3=O)C(=O)O2)cc1]

In some embodiments, LBM is

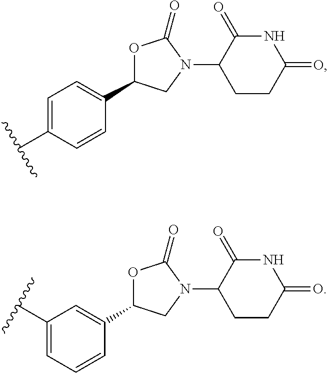 [SMILES: *c1ccc([C@@H]2CN(C3CCC(=O)NC3=O)C(=O)O2)cc1 ; *c1cccc([C@H]2CN(C3CCC(=O)NC3=O)C(=O)O2)c1]

In some embodiments, LBM is

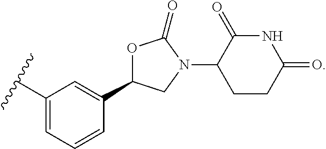 [SMILES: *c1cccc([C@@H]2CN(C3CCC(=O)NC3=O)C(=O)O2)c1]

In some embodiments, LBM is

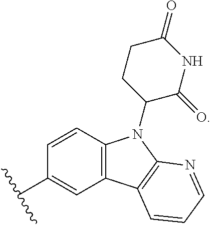 [SMILES: *c1ccc2c(c1)c1cccnc1n2C1CCC(=O)NC1=O]

In some embodiments, LBM is

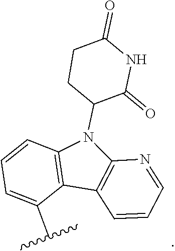 [SMILES: *c1cccc2c1c1cccnc1n2C1CCC(=O)NC1=O]

In some embodiments, LBM is

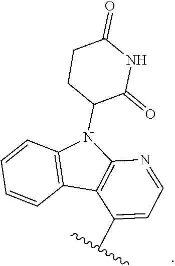 [SMILES: *c1ccnc2c1c1ccccc1n2C1CCC(=O)NC1=O]

In some embodiments, LBM is

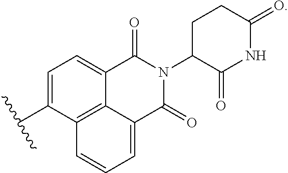 [SMILES: *c1ccc2c3c(cccc13)C(=O)N(C1CCC(=O)NC1=O)C2=O]

In some embodiments, LBM is

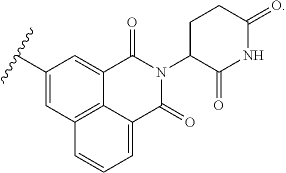 [SMILES: *c1cc2c3c(cccc3c1)C(=O)N(C1CCC(=O)NC1=O)C2=O]

In some embodiments, LBM is

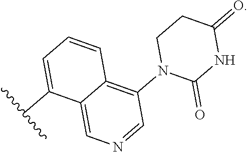 [SMILES: *c1cccc2c(N3CCC(=O)NC3=O)cncc12]

In some embodiments, LBM is

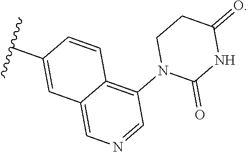 [SMILES: *c1ccc2c(N3CCC(=O)NC3=O)cncc2c1]

In some embodiments, LBM is

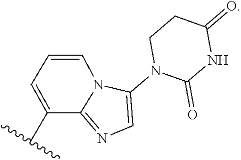 [SMILES: *c1cccn2c(N3CCC(=O)NC3=O)cnc12]

In some embodiments, LBM is

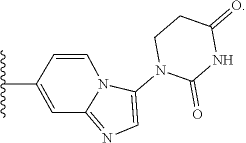 [SMILES: *c1ccn2c(N3CCC(=O)NC3=O)cnc2c1]

In some embodiments, LBM is

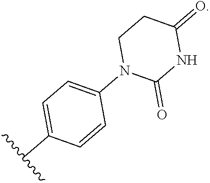 [SMILES: *c1ccc(N2CCC(=O)NC2=O)cc1]

In some embodiments, LBM is

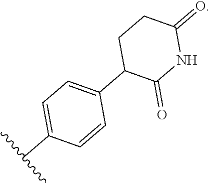 [SMILES: *c1ccc(C2CCC(=O)NC2=O)cc1]

In some embodiments, LBM is

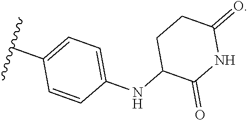 [SMILES: *c1ccc(NC2CCC(=O)NC2=O)cc1]

In some embodiments, LBM is

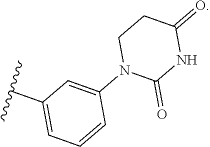 [SMILES: *c1cccc(N2CCC(=O)NC2=O)c1]

In some embodiments, LBM is

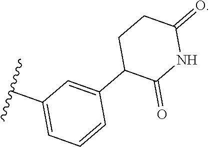 [SMILES: *c1cccc(C2CCC(=O)NC2=O)c1]

In some embodiments, LBM is

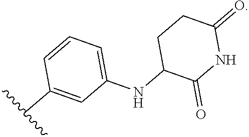 [SMILES: *c1cccc(NC2CCC(=O)NC2=O)c1]

In certain embodiments, the present invention provides a compound of Formula I, wherein LBM is a MDM2 (i.e. human double minute 2 or HDM2) E3 ligase binding moiety thereby forming a compound of formula I-i-1, I-i-2, I-i-3, I-i-4, I-i-5, I-i-6, I-i-7, I-i-8, I-i-9, I-i-10, I-i-11, I-i-12, I-i-13, I-i-14, I-i-15, I-i-16, I-i-17, or I-i-18 respectively:

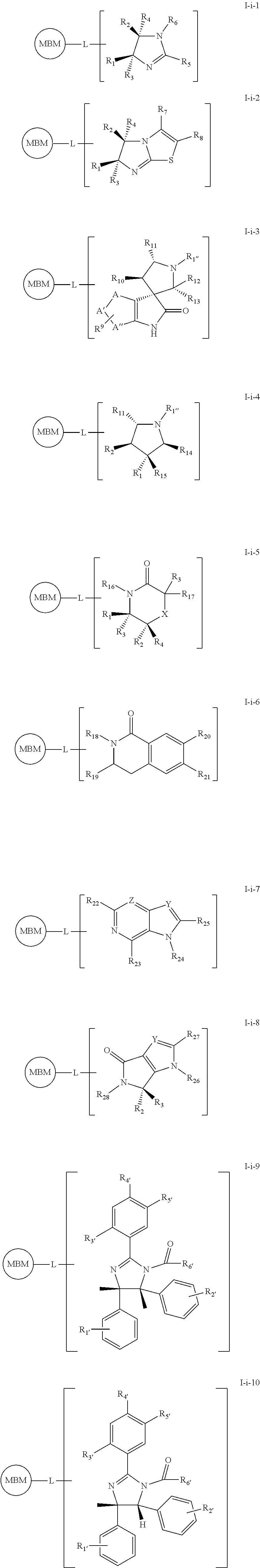

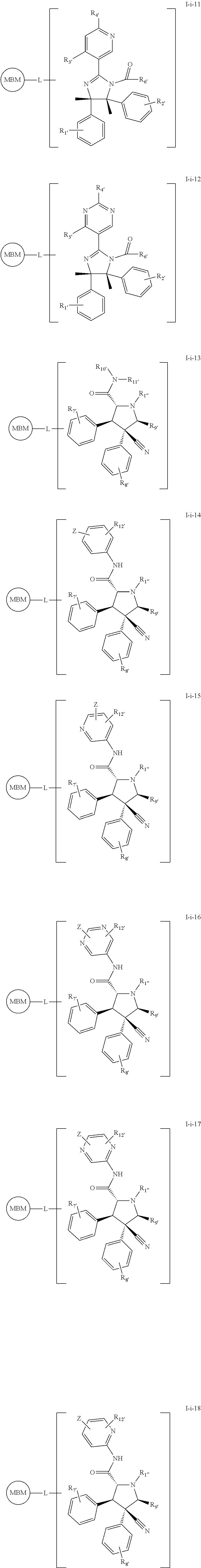

or a pharmaceutically acceptable salt thereof, wherein L and MBM are as defined above and described in embodiments herein, and wherein:
X is selected from —CR₂—, —O—, —S—, —S(O)—, —S(O)₂—, and —NR—;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom from which they are attached, independently selected from nitrogen, oxygen, and sulfur.
Y and Z are independently selected from —CR═ and —N═;
Ring W is fused ring selected from benzo and a 5-6 membered heteroaryl with 1-4 heteroatoms independently selected from nitrogen, oxygen and sulfur;
R¹ and R² are independently an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R³ and R⁴ are independently selected from hydrogen and C₁₋₆ alkyl;
R⁵ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R⁶ is selected from hydrogen, —C(O)R, —C(O)OR, and —C(O)NR₂;
R⁷ is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁸ is selected from —C(O)R and R^{A};
R⁹ is a mono-, bis-, or tri-substituent on Ring W, wherein each of the substituents are independently selected from halogen and an optionally substituted C₁₋₆ aliphatic;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R¹¹ is —C(O)OR or —C(O)NR₂;
R¹² and R¹³ are independently selected from hydrogen and R^{A}, or:
   R¹² and R³ are optionally taken together with their intervening atoms to form an optionally substituted 3-8 membered saturated, partially unsaturated, carbocyclic or heterocyclic ring having 1-3 heteroatoms independently selected from nitrogen, oxygen, and sulfur;
R¹⁴ is R^{A};
R¹⁵ is —CN;
R¹⁶ is selected from R^{A}, —OR, —(CR₂)₀₋₆—C(O)R, —(CR₂)₀₋₆—C(O)OR, —(CR₂)₀₋₆—C(O)NR₂, —(CR₂)₀₋₆—S(O)₂R, —(CR₂)₀₋₆—N(R)S(O)₂R, —(CR₂)₀₋₆—S(O)₂NR₂;
R¹⁷ is selected from —(CR₂)₀₋₆—C(O)NR₂;
R¹⁸ and R¹⁹ are independently selected from hydrogen and R^{A};
R²⁰ and R²¹ are independently selected from hydrogen, R^{A}, halogen, and —OR, or:
   R²⁰ and R²¹ are optionally taken together with their intervening atoms to form a fused 5-7 membered partially unsaturated carbocyclic or heterocyclic ring having 1-3 heteroatoms independently selected from nitrogen, oxygen, and sulfur, or a fused 5-6 membered heteroaryl ring having 1-3 heteroatoms independently selected from nitrogen, oxygen, and sulfur;
R²², R²³, R²⁵, and R²⁷ are independently selected from hydrogen, R^{A}, halogen, —C(O)R, —C(O)OR, —C(O)NR₂, —NR₂, —OR, —S(O)R, —S(O)₂R, —S(O)₂NR₂;
R²⁴, R²⁶, and R²⁸ are independently selected from hydrogen, R^{A}, —C(O)R, —C(O)OR, —C(O)NR₂, —S(O)R, —S(O)₂R, and —S(O)₂NR₂;
R^{1′} and R^{2′} are independently selected from halogen, —C≡CR, —CN, —CF₃, and —NO₂;
R^{3′} is —OR;
R^{4′}, R^{5′}, R^{6′} are independently selected from hydrogen, halogen, R^{A}, —CN, —CF₃, —NR₂, —OR, —SR, and —S(O)₂R;
R^{7′} is a mono-, bis-, or tri-substituent, wherein each of the substituents are independently selected from halogen;
R^{8′} is a mono-, bis-, or tri-substituent, wherein each of the substituents are independently selected from hydrogen, halogen, R^{A}, —CN, —C≡CR, —NO₂, and —OR;
R^{9′} is R^{A};
Z¹ is selected from hydrogen, halogen, and —OR;
R^{10′} and R^{11′} are independently selected from hydrogen and R^{A};
R^{12′} is selected from —C(O)R, —C(O)OR, —C(O)NR₂, —OR, —S(O)₂R, —S(O)₂NR₂, and —S(O)R; and
R^{1″} is selected from hydrogen and R^{A}.

In certain embodiments, the present invention provides a compound of Formula I, wherein LBM is a MDM2 (i.e. human double minute 2 or HDM2) E3 ligase binding moiety thereby forming a compound of formula I-i-19, I-i-20 or I-i-21 respectively:

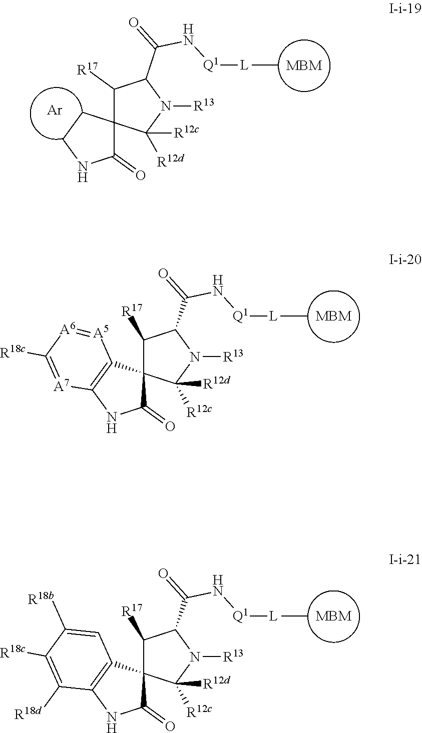

or a pharmaceutically acceptable salt thereof, wherein L and MBM are as defined above and described in embodiments herein, and wherein:
R^{1″} is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R₁₂ and R₁₃ are each independently selected from hydrogen and A, or:
   R¹² and R¹³ are optionally taken together with their intervening atoms to form an optionally substituted 4-8 membered saturated, partially unsaturated, carbocyclic or heterocyclic ring having 1-3 heteroatoms independently selected from nitrogen, oxygen, and sulfur;
A⁵ is selected from —C(R^{18a})═ and —N═;
A⁶ is selected from —C(R^{18b})═ and —N═;
A⁷ is selected from —C(R^{18d})═ and —N═;
R^{18a}, R^{18b}, R^{18c}, and R^{18d} are each independently selected from hydrogen, halogen, R^{A}, and —OR;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring W is an optionally substituted fused ring selected from benzo and a 5-6 membered heteroaryl with 1-4 heteroatoms independently selected from nitrogen, oxygen and sulfur; and
Q¹ is and optionally substituted bivalent group selected from alkylenyl, phenylenyl, heteroarylenyl, cycloalkylenyl, and heterocyclenyl.

In certain embodiments, the present invention provides a compound of formula I, wherein LBM is an IAP E3 ubiquitin ligase binding moiety thereby forming a compound of formula I-j-1, I-j-2, I-j-3, or I-j-4 respectively:

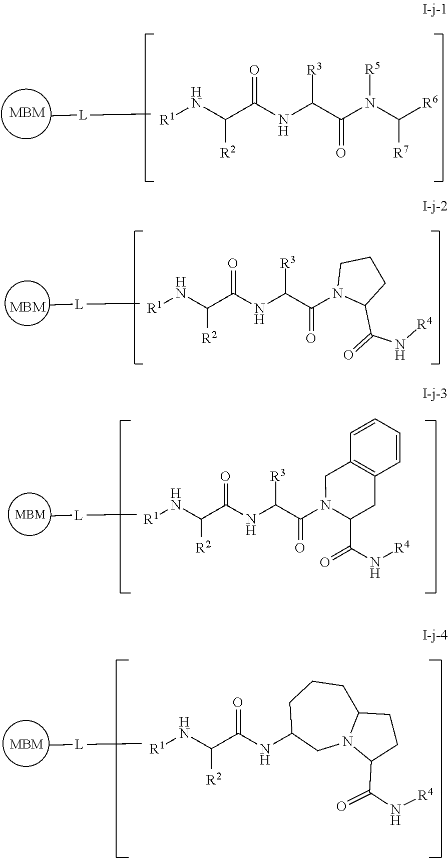

or a pharmaceutically acceptable salt thereof, wherein L and MBM are as defined above and described in embodiments herein, and wherein each of the variables R¹, R², R³, R⁴, R⁵, R⁶, and R⁷, is as defined and described in WO 2017/011590 and US 2017/0037004, the entirety of each of which is herein incorporated by reference.

In certain embodiments, the present invention provides a compound of formula I, wherein LBM is an IAP binding moiety thereby forming a compound of formula I-k-1:

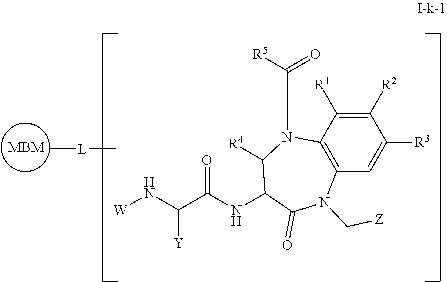

or a pharmaceutically acceptable salt thereof, wherein L and MBM are as defined above and described in embodiments herein, and wherein each of the variables W, Y, Z, R¹, R², R³, R⁴, and R is as described and defined in WO 2014/044622, US 2015/0225449. WO 2015/071393, and US 2016/0272596, the entirety of each of which is herein incorporated by reference.

In certain embodiments, the present invention provides a compound of formula I, wherein LBM is a DCAF16 binding moiety thereby forming a compound of formula I-k-2:

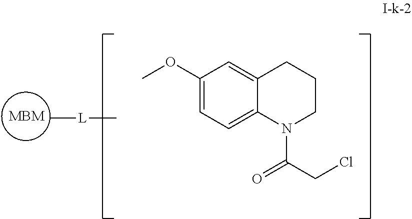

or a pharmaceutically acceptable salt thereof as described and defined in Zhang, X. et al., bioRxiv (doi: https://doi.org/10.1101/443804), the entirety of each of which is herein incorporated by reference, and wherein L and MBM are as defined above and described in embodiments herein.

In certain embodiments, the present invention provides a compound of formula I, wherein LBM is a RNF114 binding moiety thereby forming a compound of formula I-k-3:

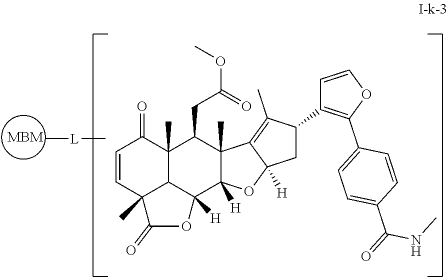

or a pharmaceutically acceptable salt thereof, as described and defined in Spradin, J. N. et al., bioRxiv (doi: https://doi.org/10.1101/436998), the entirety of each of which is herein incorporated by reference, and wherein L and MBM are as defined above and described in embodiments herein.

In certain embodiments, the present invention provides a compound of formula I, wherein LBM is a RNF4 binding moiety thereby forming a compound of formula I-k-4:

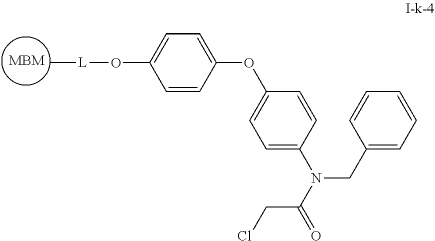

or a pharmaceutically acceptable salt thereof, as described and defined in Ward, C. C., et al., bioRxiv (doi: https://doi.org/10.1101/439125), the entirety of each of which is herein incorporated by reference, and wherein L and MBM are as defined above and described in embodiments herein.

In certain embodiments, the present invention provides a compound of formula I, wherein LBM is a E3 ubiquitin ligase (cereblon) binding moiety thereby forming a compound of formula I-l-1, I-l-2, I-l-3, or I-l-4:

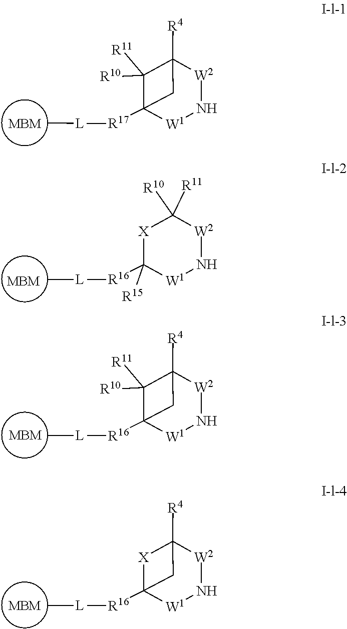

or a pharmaceutically acceptable salt thereof, wherein L and MBM are as defined above and described herein, and wherein each of the variables R⁴, R¹⁰, R¹¹, R¹⁵, R¹⁶, R¹⁷, W¹, W², and X is as defined in WO 2019/099868 which is herein incorporated by reference in its entirety, and wherein

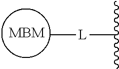

is attached to R¹⁷ or R¹⁶ at the site of attachment of R¹² as defined in WO 2018/237026, such that

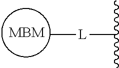

takes the place of the R¹² substituent.

In certain embodiments, the present invention provides a compound of formula I, wherein LBM is a cereblon E3 ubiquitin ligase binding moiety, a DCAF15 E3 ubiquitin ligase binding moiety, or a VHL E3 ubiquitin ligase binding moiety; thereby forming a compound of formula I-m-1, I-m-2, or I-m-3:

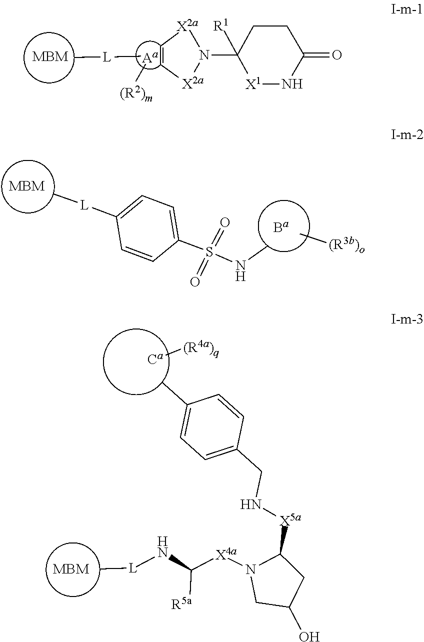

or a pharmaceutically acceptable salt thereof, wherein L and MBM is as defined above and described in embodiments herein, and wherein:
each of X¹, X^{2a}, and X^{3a} is independently a bivalent moiety selected from a covalent bond, —CH₂—, —C(O)—, —C(S)—, or

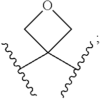 [SMILES: *C1(*)COC1]

each of X^{4a} and X^{5a} is independently a bivalent moiety selected from —CH₂—, —C(O)—, —C(S)—, or

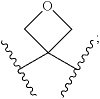 [SMILES: *C1(*)COC1]

R¹ is hydrogen, halogen, —CN, —OR, —SR, —S(O)R, —S(O)₂R, —NR₂, or an optionally substituted C₁₋₄ aliphatic;
each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R^{5a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen and sulfur, or 5-membered heteroaryl with 1-3 heteroatoms independently selected from nitrogen, oxygen and sulfur;
Ring B^{a} is selected from 6-membered aryl containing 0-2 nitrogen atoms or a 8-10 membered bicyclic heteroaryl having 1-5 heteroatoms independently selected from nitrogen, oxygen, and sulfur;
Ring C^{a} is a selected from 6-membered aryl containing 0-2 nitrogen atoms or a 5-membered heteroaryl with 1-3 heteroatoms independently selected from nitrogen, oxygen and sulfur;
m is 0, 1, 2, 3 or 4;
is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of formula I-m-1, wherein LBM is an E3 ubiquitin ligase (cereblon) binding moiety thereby forming a compound of formula I-m-4 or I-m-5:

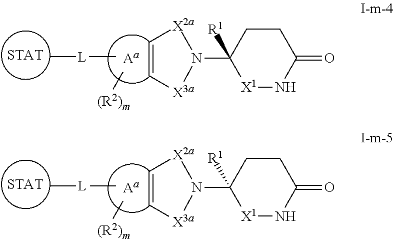

or a pharmaceutically acceptable salt thereof, wherein MBM, L, Ring A^{a}, X¹, X^{2a}, X^{3a}, R¹, R² and m are as described above.

As defined above and described herein, each of X¹, X^{2a}, and X^{3a} is independently a bivalent moiety selected from a covalent bond, —CH₂—, —C(O)—, —C(S)—, or

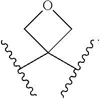 [SMILES: *C1(*)COC1]

In some embodiments, X¹ is a covalent bond, —CH₂—, —C(O)—, —C(S)—, or

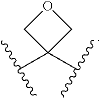 [SMILES: *C1(*)COC1]

In some embodiments, X¹ is selected from those depicted in Table 1, below.

In some embodiments, X^{2a} is a covalent bond, —CH₂—, —C(O)—, —C(S)—, or

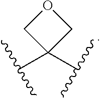 [SMILES: *C1(*)COC1]

In some embodiments, X^{2a} is selected from those depicted in Table 1, below.

In some embodiments, X^{3a} is a covalent bond, —CH₂—, —C(O)—, —C(S)—, or

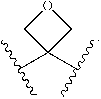 [SMILES: *C1(*)COC1]

In some embodiments, X^{3a} is selected from those depicted in Table 1, below.

As defined above and described herein, each of X^{4a} and X^{5a} is independently a bivalent moiety selected from —CH₂—, —C(O)—, —C(S)—, or

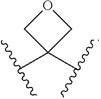 [SMILES: *C1(*)COC1]

In some embodiments, X^{4a} is —CH₂—, —C(O)—, —C(S)—, or

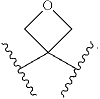 [SMILES: *C1(*)COC1]

In some embodiments, X^{4a} is selected from those depicted in Table 1, below.

In some embodiments, X^{5a} is —CH₂—, —C(O)—, —C(S)—, or

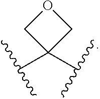 [SMILES: *C1(*)COC1]

In some embodiments, X^{5a} is selected from those depicted in Table 1, below.

As defined above and described herein, R¹ is hydrogen, halogen, —CN, —OR, —SR, —S(O)R, —S(O)₂R, —NR₂, or an optionally substituted C₁₋₄ aliphatic.

In some embodiments, R¹ is hydrogen. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is optionally substituted C₁₋₄ aliphatic.

In some embodiments, R¹ is selected from those depicted in Table 1, below.

As defined above and described herein, each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{z} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is selected from those depicted in Table 1, below.

In some embodiments, R^{3b} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{3b} is methyl.

In some embodiments, R^{3b} is selected from those depicted in Table 1, below.

In some embodiments, R^{4a} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{4a} is methyl.

In some embodiments, R^{4a} is selected from those depicted in Table 1, below.

As defined above and described herein, R^{a} is hydrogen or C₁₋₆ aliphatic.

In some embodiments, R^{5a} is t-butyl.

In some embodiments, R^{5a} is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁶ is an optionally substituted C₁₋₆ aliphatic group. In some embodiments, R⁶ is an optionally substituted phenyl. In some embodiments, R⁶ is an optionally substituted 4-7 membered saturated or partially unsaturated heterocyclic ring having 1-2 heteroatoms independently selected from nitrogen, oxygen, and sulfur. In some embodiments, R⁶ is an optionally substituted 5-6 membered heteroaryl ring having 1-4 heteroatoms independently selected from nitrogen, oxygen, and sulfur.

In some embodiments, R⁶ is selected from those depicted in Table 1, below.

As defined above and described herein, Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen and sulfur, or 5-membered heteroaryl with 1-3 heteroatoms independently selected from nitrogen, oxygen and sulfur.

In some embodiments Ring A^{a} is a fused 6-membered aryl containing 0-2 nitrogen atoms. In some embodiments Ring A^{a} is a fused 5 to 7-membered partially saturated carbocyclyl. In some embodiments Ring A^{a} is a fused 5 to 7-membered partially saturated heterocyclyl with 1-2 heteroatoms independently selected from nitrogen, oxygen and sulfur. In some embodiments Ring A^{a} is a fused 5-membered heteroaryl with 1-3 heteroatoms independently selected from nitrogen, oxygen and sulfur.

In some embodiments, Ring A^{a} is a fused phenyl.

In some embodiments, Ring A^{a} is selected from those depicted in Table 1, below.

As defined above and described herein, Ring B^{a} is selected from 6-membered aryl containing 0-2 nitrogen atoms or a 8-10 membered bicyclic heteroaryl having 1-5 heteroatoms independently selected from nitrogen, oxygen, and sulfur.

In some embodiments, Ring B^{a} is a 6-membered aryl containing 0-2 nitrogen atoms. In some embodiments, Ring B^{a} is a 8-10 membered bicyclic heteroaryl having 1-5 heteroatoms independently selected from nitrogen, oxygen, and sulfur.

In some embodiments, Ring B^{a} is

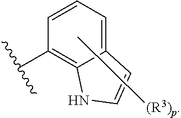

In some embodiments, Ring B^{a} is selected from those depicted in Table 1, below.

As defined above and described herein, Ring C^{a} is selected from 6-membered aryl containing 0-2 nitrogen atoms or a 5-membered heteroaryl with 1-3 heteroatoms independently selected from nitrogen, oxygen and sulfur.

In some embodiments, Ring C^{a} is a 6-membered aryl containing 0-2 nitrogen atoms. In some embodiments, Ring C^{a} is a 5-membered heteroaryl with 1-3 heteroatoms independently selected from nitrogen, oxygen and sulfur.

In some embodiments, Ring C^{a} is

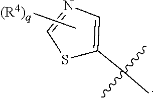

In some embodiments, Ring C^{a} is selected from those depicted in Table 1, below.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In some embodiments, m is selected from those depicted in Table 1, below.

In some embodiments, o is selected from those depicted in Table 1, below.

As defined above and described herein, o is 0, 1, 2, 3 or 4.

In some embodiments, o is 0. In some embodiments, o is 1. In some embodiments, o is 2. In some embodiments, o is 3. In some embodiments, o is 4.

In some embodiments, o is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is phenyl. In some embodiments, R is a 4-7 membered saturated or partially unsaturated heterocyclic having 1-2 heteroatoms independently selected from nitrogen, oxygen, and sulfur. In some embodiments, R is a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1, below.

In certain embodiments, the present invention provides a compound of formula I, wherein LBM is a VHL E3 ubiquitin ligase binding moiety, thereby forming a compound of formula I-n:

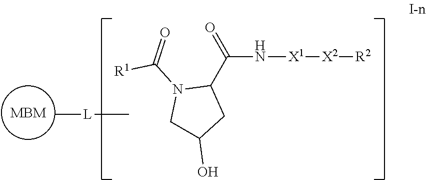

or a pharmaceutically acceptable salt thereof, wherein L and MBM is as defined above and described in embodiments herein, and wherein:
X¹ is a bivalent group selected from —O—, —C(O)—, —C(S)—, —C(R)₂—, —NR—, —S(O)—, —SO₂— or an optionally substituted 5-membered heterocyclylene;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{z}, —C(R)₂R^{z}, —OR, —SR, —N(R)₂, —C(R)₂, —C(R)₂OR, —C(R)₂NR₂, —C(R)₂NRC(O)R, —C(R)₂NRC(O)N(R)₂, —OCR₂, —NRC(O)OR, —NRC(O)R, —NRC(O)N(R)₂, or —NRSO₂R;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are taken together with their intervening atoms to form an optionally substituted 3-7 membered saturated or partially unsaturated carbocyclic ring or an optionally substituted 3-7 membered saturated, partially unsaturated, or heteroaryl ring having 0-3 heteroatoms, in addition to the atom to which they are attached, independently selected from nitrogen, oxygen, and sulfur;
   each R^{z} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R² is hydrogen or

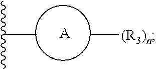

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur, wherein Ring A is optionally further substituted with 1-2 oxo groups;
each of R³ is independently hydrogen, R^{z}, halogen, —CN, —NO₂, —OR, —SR, —N(R)₂, —Si(R)₃, —SO₂R, —SO₂NR₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)NR₂, —OP(O)R₂, —OP(O)(OR)₂, —OP(O)(OR)N(R)₂, —OP(O)(NR₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(NR₂)₂, or —N(R)SO₂R; or
   two R³ groups are optionally taken together to form an optionally substituted 5-7 membered partially unsaturated or aryl fused ring having 0-2 heteroatoms independently selected from nitrogen, oxygen, and sulfur;
n is 0, 1, 2, 4, or 5.

In some embodiments, LBM is

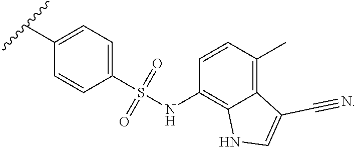 [SMILES: *c1ccc(S(=O)(=O)Nc2ccc(C)c3c(C#N)c[nH]c23)cc1]

some embodiments, LBM is

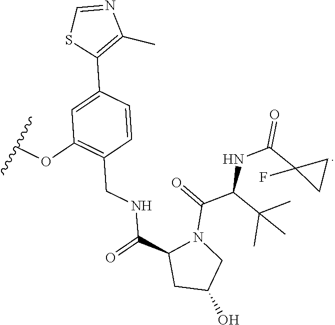 [SMILES: *Oc1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

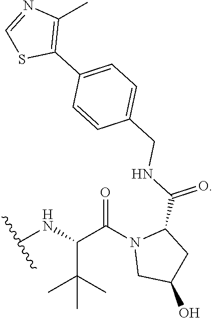 [SMILES: *N[C@H](C(=O)N1C[C@H](O)C[C@H]1C(=O)NCc1ccc(-c2scnc2C)cc1)C(C)(C)C]

In some embodiments, LBM is

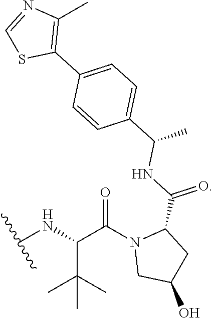 [SMILES: *N[C@H](C(=O)N1C[C@H](O)C[C@H]1C(=O)N[C@@H](C)c1ccc(-c2scnc2C)cc1)C(C)(C)C]

In some embodiments, LBM is

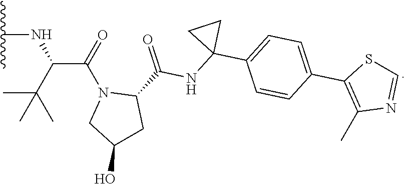 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

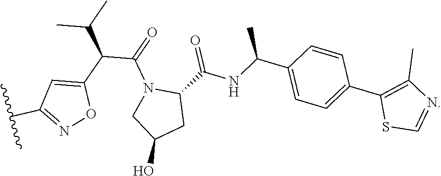 [SMILES: *c1cc([C@H](C(=O)N2C[C@H](O)C[C@H]2C(=O)N[C@@H](C)c2ccc(-c3scnc3C)cc2)C(C)C)on1]

In some embodiments, LBM is

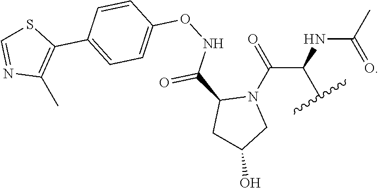 [SMILES: *[C@H](NC(C)=O)C(=O)N1C[C@H](O)C[C@H]1C(=O)NOc1ccc(-c2scnc2C)cc1]

In some embodiments, LBM is

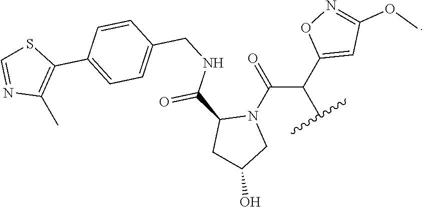 [SMILES: *C(C(=O)N1C[C@H](O)C[C@H]1C(=O)NCc1ccc(-c2scnc2C)cc1)c1cc(OC)no1]

In some embodiments, LBM is

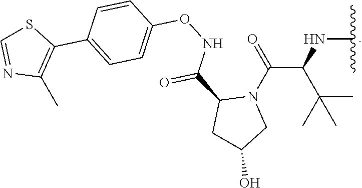 [SMILES: *N[C@H](C(=O)N1C[C@H](O)C[C@H]1C(=O)NOc1ccc(-c2scnc2C)cc1)C(C)(C)C]

In some embodiments, LBM is

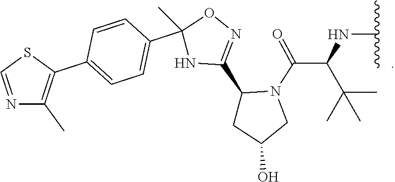 [SMILES: *N[C@H](C(=O)N1C[C@H](O)C[C@H]1C1=NOC(C)(c2ccc(-c3scnc3C)cc2)N1)C(C)(C)C]

In some embodiments, LBM is

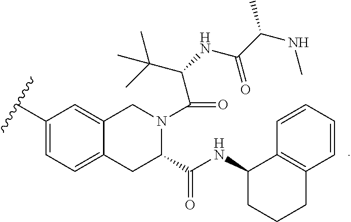 [SMILES: *c1ccc2c(c1)CN(C(=O)[C@@H](NC(=O)[C@H](C)NC)C(C)(C)C)[C@H](C(=O)N[C@@H]1CCCc3ccccc31)C2]

In some embodiments, LBM is

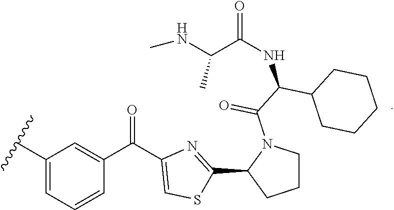 [SMILES: *c1cccc(C(=O)c2csc([C@@H]3CCCN3C(=O)[C@@H](NC(=O)[C@H](C)NC)C3CCCCC3)n2)c1]

In some embodiments, LBM is

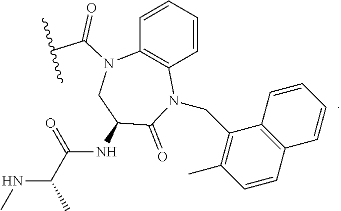 [SMILES: *C(=O)N1C[C@H](NC(=O)[C@H](C)NC)C(=O)N(Cc2c(C)ccc3ccccc23)c2ccccc21]

In some embodiments, LBM is

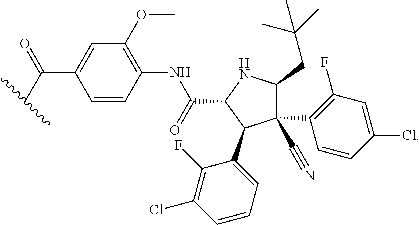 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]

In some embodiments, LBM is

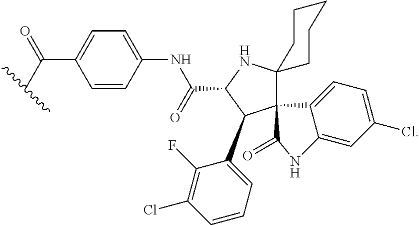 [SMILES: *C(=O)c1ccc(NC(=O)[C@@H]2NC3(CCCCC3)[C@@]3(C(=O)Nc4cc(Cl)ccc43)[C@H]2c2cccc(Cl)c2F)cc1]

In some embodiments, LBM is

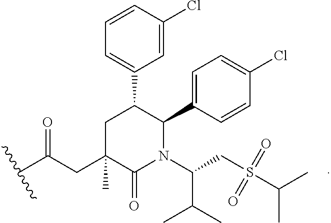 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O]

In some embodiments, LBM is

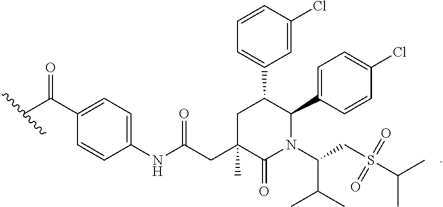 [SMILES: *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

In some embodiments, LBM is

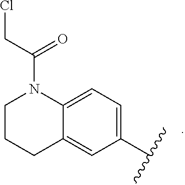 [SMILES: *c1ccc2c(c1)CCCN2C(=O)CCl]

In some embodiments, LBM is

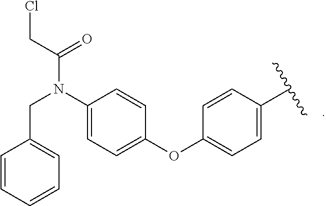 [SMILES: *c1ccc(Oc2ccc(N(Cc3ccccc3)C(=O)CCl)cc2)cc1]

In some embodiments, LBM is

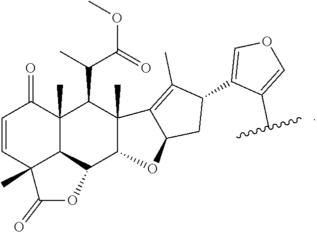 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45]

In some embodiments, LBM is

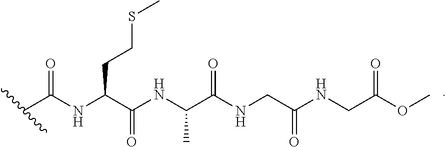 [SMILES: *C(=O)N[C@@H](CCSC)C(=O)N[C@@H](C)C(=O)NCC(=O)NCC(=O)OC]

In some embodiments, LBM is

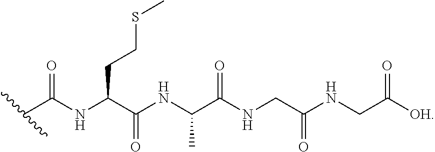 [SMILES: *C(=O)N[C@@H](CCSC)C(=O)N[C@@H](C)C(=O)NCC(=O)NCC(=O)O]

In some embodiments, LBM is

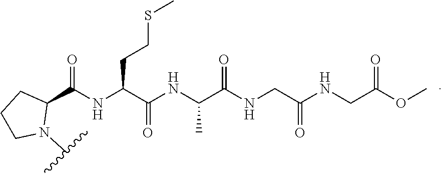 [SMILES: *N1CCC[C@H]1C(=O)N[C@@H](CCSC)C(=O)N[C@@H](C)C(=O)NCC(=O)NCC(=O)OC]

In some embodiments, LBM is

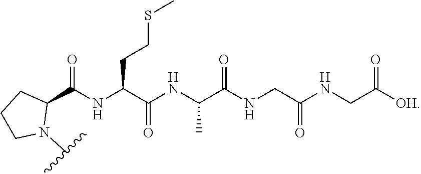 [SMILES: *N1CCC[C@H]1C(=O)N[C@@H](CCSC)C(=O)N[C@@H](C)C(=O)NCC(=O)NCC(=O)O]

In some embodiments, LBM is

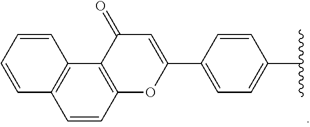 [SMILES: *c1ccc(-c2cc(=O)c3c(ccc4ccccc43)o2)cc1]

In some embodiments, LBM is

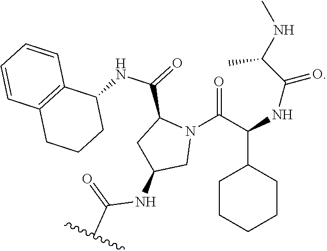 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1]

In some embodiments, LBM is

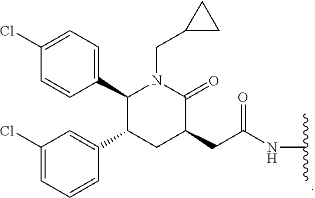 [SMILES: *NC(=O)C[C@H]1C[C@H](c2cccc(Cl)c2)[C@@H](c2ccc(Cl)cc2)N(CC2CC2)C1=O]

In some embodiments, LBM is

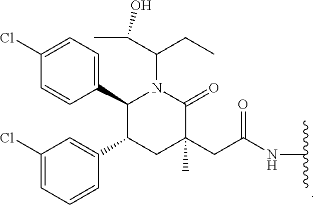 [SMILES: *NC(=O)C[C@@]1(C)C[C@H](c2cccc(Cl)c2)[C@@H](c2ccc(Cl)cc2)N(C(CC)[C@H](C)O)C1=O]

In some embodiments, LBM is

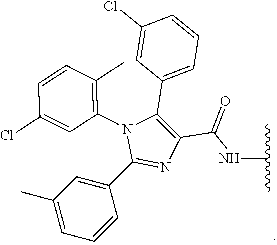 [SMILES: *NC(=O)c1nc(-c2cccc(C)c2)n(-c2cc(Cl)ccc2C)c1-c1cccc(Cl)c1]

In some embodiments, LBM is

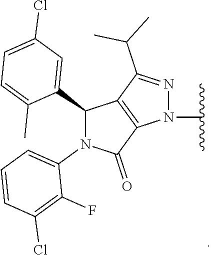 [SMILES: *n1nc(C(C)C)c2c1C(=O)N(c1cccc(Cl)c1F)[C@H]2c1cc(Cl)ccc1C]

In certain embodiments, the present invention provides a compound of formula I, wherein LBM is a E3 ubiquitin ligase (cereblon) binding moiety thereby forming a compound of formula formula I-ll:

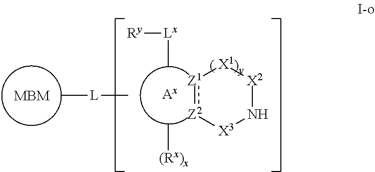

or a pharmaceutically acceptable salt thereof, wherein L and MBM are as defined above and described in embodiments herein, wherein:
each X¹ is independently —CH₂—, —O—, —NR—, —CF₂—,

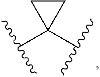 [SMILES: *C1(*)CC1]

### —C(O)—, —C(S)—, or

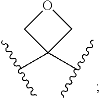 [SMILES: *C1(*)COC1] [0922] X² and X³ are independently —CH₂—, —C(O)—, —C(S)—, or

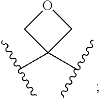 [SMILES: *C1(*)COC1]

Z¹ and Z² are independently a carbon atom or a nitrogen atom;
Ring A^{x} is a fused ring selected from benzo or a 5-6 membered heteroaryl ring having 1-4 heteroatoms independently selected from nitrogen, oxygen, and sulfur;
L^{x} is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R^{x} is independently selected from hydrogen, R^{z}, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
   two R^{x} groups are optionally taken together to form an optionally substituted 5-8 membered partially unsaturated or aryl fused ring having 0-2 heteroatoms independently selected from nitrogen, oxygen, and sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R^{y} is selected from

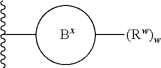

or hydrogen;
Ring B^{x} is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B^{x} is further optionally substituted with 1-2 oxo groups;
each R^{w} is independently selected from hydrogen, R^{z}, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
each R^{z} is independently selected from an optionally substituted group selected from C₁₋₆ aliphatic, phenyl, a 4-7 membered saturated or partially unsaturated heterocyclic ring having 1-2 [symbol] heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
x is 0, 1, 2, 3 or 4;
y is 0, 1 or 2; and
w is 0, 1, 2, 3 or 4.

As defined above and described herein, each X¹ is independently —CH₂—, —O—, —NR—, —CF₂—,

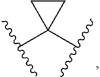 [SMILES: *C1(*)CC1]

### —C(O)—, —C(S)—, or

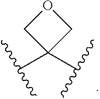 [SMILES: *C1(*)COC1]

In some embodiments, X is a covalent bond. In some embodiments, X¹ is —CH₂—. In some embodiments, X¹ is —O—. In some embodiments, X¹ is —NR—. In some embodiments, X¹ is —CF₂—. In some embodiments, X¹ is

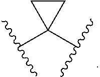 [SMILES: *C1(*)CC1]

In some embodiments, X¹ is —C(O)—. In some embodiments, X¹ is —C(S)—. In some embodiments, X¹ is

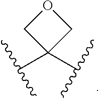 [SMILES: *C1(*)COC1]

In certain embodiments, X¹ is selected from those shown in the compounds of Table 1.

As defined above and described herein, X² and X³ are independently —CH₂—, —C(O)—, —C(S)—, or

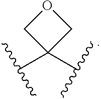 [SMILES: *C1(*)COC1]

In some embodiments, X² and X³ are independently —CH₂—. In some embodiments, X² and X³ are independently —C(O)—. In some embodiments, X² and X³ are independently —C(S)—. In some embodiments, X² and X³ are independently

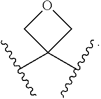 [SMILES: *C1(*)COC1]

In certain embodiments, X² and X³ are independently selected from those shown in the compounds of Table 1.

As define above and described herein, Z¹ and Z² are independently a carbon atom or a nitrogen atom.

In some embodiments, Z¹ and Z² are independently a carbon atom. In some embodiments, Z¹ and Z² are independently a carbon atom.

In certain embodiments, Z¹ and Z² are independently selected from those shown in the compounds of Table 1.

As defined above and described herein, Ring A^{x} is fused ring selected from benzo or a 5-6 membered heteroaryl ring having 1-4 heteroatoms independently selected from nitrogen, oxygen, and sulfur.

In some embodiments, Ring A^{x} is benzo. In some embodiments, Ring A^{x} is a 5-6 membered heteroaryl ring having 1-4 heteroatoms independently selected from nitrogen, oxygen, and sulfur.

In some embodiments, Ring A^{x} is

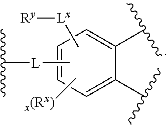

In some embodiments, Ring A^{x} is

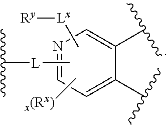

In certain embodiments, Ring A^{x} is selected from those shown in the compounds of Table 1.

As defined above and described herein, L^{x} is a covalent bond or a C₁_3 bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L^{x} is a covalent bond. In some embodiments, L^{x} is a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L^{x} is —C(O)—.

In certain embodiments, L^{x} is selected from those shown in the compounds of Table 1.

As defined above and described herein, each R^{x} is independently selected from hydrogen, R^{z}, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃, or two R^{x} groups are optionally taken together to form an optionally substituted 5-8 membered partially unsaturated or aryl fused ring having 0-2 heteroatoms independently selected from nitrogen, oxygen, and sulfur.

In some embodiments, R^{x} is hydrogen. In some embodiments, R^{x} is R^{z}. In some embodiments, R^{x} is halogen. In some embodiments, R^{x} is —CN. In some embodiments, R^{x} is —NO₂. In some embodiments, R^{x} is —OR. In some embodiments, R^{x} is —SR. In some embodiments, R^{x} is —NR₂. In some embodiments, R^{x} is —S(O)₂R. In some embodiments, R^{x} is —S(O)₂NR₂. In some embodiments, R^{x} is —S(O)R. In some embodiments, R^{x} is —CF₂R. In some embodiments, R is —CF₃. In some embodiments, R^{x} is —CR₂(OR). In some embodiments, R is —CR₂(NR₂). In some embodiments, R^{x} is —C(O)R. In some embodiments, R is —C(O)OR. In some embodiments, R^{x} is —C(O)NR₂. In some embodiments, R^{x} is —C(O)N(R)OR. In some embodiments, R^{x} is —OC(O)R. In some embodiments, R is —OC(O)NR₂. In some embodiments, R^{x} is —C(S)NR₂. In some embodiments, R^{x} is —N(R)C(O)OR. In some embodiments, R^{x} is —N(R)C(O)R. In some embodiments, R^{x} is —N(R)C(O)NR₂. In some embodiments, R^{x} is —N(R)S(O)₂R. In some embodiments, R^{x} is —OP(O)R₂. In some embodiments, R^{x} is —OP(O)(OR)₂. In some embodiments, R^{x} is —OP(O)(OR)NR₂. In some embodiments, R^{x} is —OP(O)(NR₂)₂. In some embodiments, R^{x} is —Si(OR)R₂. In some embodiments, R^{x} is —SiR₃. In some embodiments, two R^{x} groups are optionally taken together to form an optionally substituted 5-8 membered partially unsaturated or aryl fused ring having 0-2 heteroatoms independently selected from nitrogen, oxygen, and sulfur.

In certain embodiments, each R^{x} is independently selected from those shown in the compounds of Table 1.

As defined above and described here, each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

As defined above and described herein, R^{y} is selected from

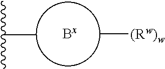

or hydrogen.

In some embodiment R^{y} is

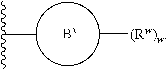

In some embodiments, R^{y} is hydrogen.

In certain embodiments, R^{y} is selected from those shown in the compounds of Table 1.

As defined above and described herein, Ring B^{x} is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B^{x} is further optionally substituted with 1-2 oxo groups.

In some embodiments, Ring B^{x} is phenyl. In some embodiments, Ring B^{x} is a 4-10 membered saturated or partially unsaturated mono- or bicyclic carbocyclic or heterocyclic ring having 1-3 heteroatoms independently selected from nitrogen, oxygen, and sulfur In some embodiments, Ring B^{x} is a 5-6 membered heteroaryl ring having 1-4 heteroatoms independently selected from nitrogen, oxygen, and sulfur. In some embodiments, Ring B^{x} is further optionally substituted with 1-2 oxo groups.

In certain embodiments, Ring B^{x} is selected from those shown in the compounds of Table 1.

As defined above and described herein, each R^{w} is independently selected from hydrogen, R^{z}, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃.

In some embodiments, R^{w} is hydrogen. In some embodiments, R^{w} is R^{z}. In some embodiments, R^{w} is halogen. In some embodiments, R^{w} is —CN. In some embodiments, R^{w} is —NO₂. In some embodiments, R^{w} is —OR. In some embodiments, R^{w} is —SR. In some embodiments, R^{w} is —NR₂. In some embodiments, R^{w} is —S(O)₂R. In some embodiments, R^{w} is —S(O)₂NR₂. In some embodiments, R^{w} is —S(O)R. In some embodiments, R^{w} is —CF₂R. In some embodiments, R^{w} is —CF₃. In some embodiments, R^{w} is —CR₂(OR). In some embodiments, R^{w} is —CR₂(NR₂). In some embodiments, R^{w} is —C(O)R. In some embodiments, R^{w} is —C(O)OR. In some embodiments, R^{w} is —C(O)NR₂. In some embodiments, R^{w} is —C(O)N(R)OR. In some embodiments, R^{w} is —OC(O)R. In some embodiments, R^{w} is —OC(O)NR₂. In some embodiments, R^{w} is —N(R)C(O)OR. In some embodiments, R^{w} is —N(R)C(O)R. In some embodiments, R^{w} is —N(R)C(O)NR₂. In some embodiments, R^{w} is —N(R)S(O)₂R. In some embodiments, R^{w} is —OP(O)R₂. In some embodiments, R^{w} is —OP(O)(OR)₂. In some embodiments, R^{w} is —OP(O)(OR)NR₂. In some embodiments, R^{w} is —OP(O)(NR₂)₂. In some embodiments, R^{w} is —SiR₃.

In certain embodiments, R^{w} is selected from those shown in the compounds of Table 1.

As defined above and described herein, each R^{z} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{z} is an optionally substituted C₁₋₆ aliphatic. In some embodiments, R^{z} is an optionally substituted phenyl. In some embodiments, R^{z} is an optionally substituted 4-7 membered saturated or partially unsaturated heterocyclic ring having 1-2 heteroatoms independently selected from nitrogen, oxygen, and sulfur. In some embodiments, R^{z} is an optionally substituted 5-6 membered heteroaryl ring having 1-4 heteroatoms independently selected from nitrogen, oxygen, and sulfur.

In certain embodiments, R^{z} is selected from those shown in the compounds of Table 1.

As defined above and described herein, [symbol] is a single or double bond.

In some embodiments, [symbol] is a single bond. In some embodiments, [symbol] is a double bond.

In certain embodiments, [symbol] is selected from those shown in the compounds of Table 1.

As defined above and described herein, w is 0, 1, 2, 3 or 4.

In some embodiments, w is 0. In some embodiments, w is 1. In some embodiments, w is 2. In some embodiments, w is 3. In some embodiments, w is 4.

In certain embodiments, w is selected from those shown in the compounds of Table 1.

As defined above and described herein, x is 0, 1, 2, 3 or 4.

In some embodiments, x is 0. In some embodiments, x is 1. In some embodiments, m is 2. In some embodiments, x is 3. In some embodiments, x is 4.

In certain embodiments, x is selected from those shown in the compounds of Table 1.

As defined above and described herein, y is 0, 1 or 2.

In some embodiments, y is 0. In some embodiments, y is 1. In some embodiments, y is 2.

In certain embodiments, y is selected from those shown in the compounds of Table 1.

In some embodiments, the present invention provides a compound of formula I-n, wherein Ring A^{x} is benzo, y is 1, X¹ is —CH₂—, X² and X³ are —C(O)—, and Z¹ and Z² are carbon atoms as shown, to provide a compound of formula I-n-1:

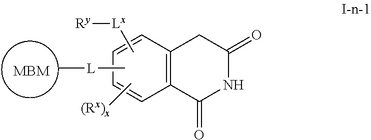

or a pharmaceutically acceptable salt thereof, wherein each of MBM, L, L^{x}, R^{x}, R^{y}, and x is as defined above and described in embodiments herein, both singly and in combination.

In some embodiments, the present invention provides a compound of formula I-n, wherein Ring A is benzo, y is 1, X¹, X² and X³ are —C(O)—, and Z¹ and Z² are carbon atoms as shown, to provide a compound of formula I-n-2:

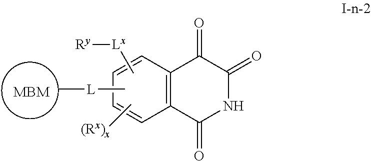

or a pharmaceutically acceptable salt thereof, wherein each of MBM, L, L^{x}, R^{x}, R^{y}, and x is as defined above and described in embodiments herein, both singly and in combination.

In some embodiments, LBM is

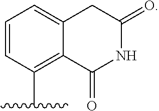 [SMILES: *c1cccc2c1C(=O)NC(=O)C2]

In some embodiments, LBM is

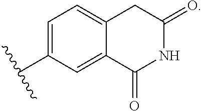 [SMILES: *c1ccc2c(c1)C(=O)NC(=O)C2]

In some embodiments, LBM is

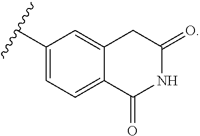 [SMILES: *c1ccc2c(c1)CC(=O)NC2=O]

In some embodiments, LBM is

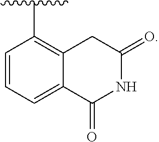 [SMILES: *c1cccc2c1CC(=O)NC2=O]

In some embodiments, LBM is

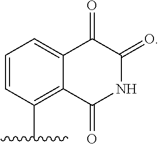 [SMILES: *c1cccc2c1C(=O)NC(=O)C2=O]

In some embodiments, LBM is

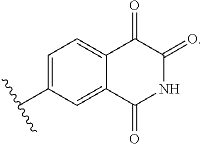 [SMILES: *c1ccc2c(c1)C(=O)NC(=O)C2=O]

In some embodiments, LBM is

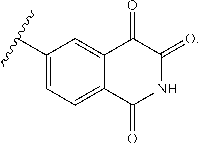 [SMILES: *c1ccc2c(c1)C(=O)C(=O)NC2=O]

In some embodiments, LBM is

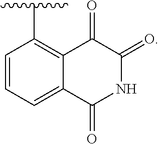 [SMILES: *c1cccc2c1C(=O)C(=O)NC2=O]

In some embodiments, LBM is selected from those in Table 1.

In certain embodiments, the present invention provides a compound of formula I, wherein LBM is a RPN13 binding moiety thereby forming a compound of formula I-o-1:

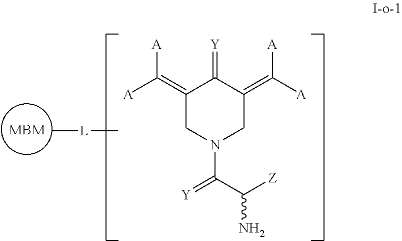

or a pharmaceutically acceptable salt thereof, wherein L and MBM are as defined above and described in embodiments herein, and wherein each of the variables A, Y, and Z is as described and defined in WO 2019/165229, the entirety of each of which is herein incorporated by reference.

In certain embodiments, the present invention provides a compound of formula I, wherein LBM is a Ubr1 binding moiety as described in Shanmugasundaram, K. et al, J. Bio. Chem. 2019, doi: 10.1074/jbc.AC119.010790, the entirety of each of which is herein incorporated by reference, thereby forming a compound of formula I-o-2 or I-o-3:

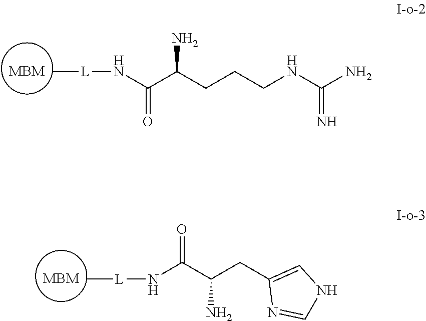

or a pharmaceutically acceptable salt thereof, wherein L and MBM are as defined above and described in embodiments herein.

In certain embodiments, the present invention provides a compound of formula I, wherein LBM is a cereblon binding moiety thereby forming a compound of formula I-o-4:

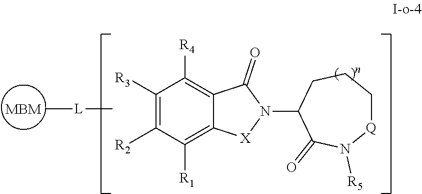

or a pharmaceutically acceptable salt thereof, wherein L and MBM are as defined above and described in embodiments herein, and wherein each of the variables R₁, R₂, R₃, R₄, R₅, Q, X, and n is as described and defined in US 2019/276474, the entirety of each of which is herein incorporated by reference.

In certain embodiments, the present invention provides a compound of formula I, wherein LBM is a cereblon E3 ubiquitin ligase binding moiety thereby forming a compound of formula I-o-5, I-o-6, I-o-7 or I-o-8:

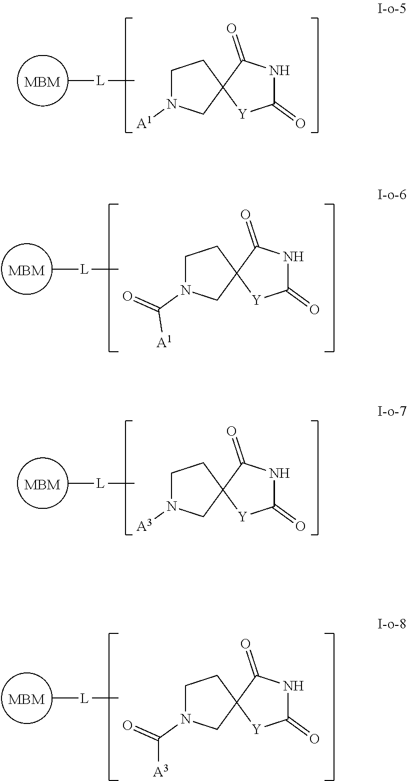

or a pharmaceutically acceptable salt thereof, wherein L and MBM are as defined above and described in embodiments herein, and wherein each of the variables Y, A¹, and A³ is as described and defined in WO 2019/236483, the entirety of each of which is herein incorporated by reference.

In certain embodiments, the present invention provides a compound of formula I, wherein LBM is human kelch-like ECH-associated protein 1 (KEAP1) of formula I-o-9:

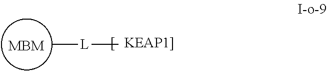

or a pharmaceutically acceptable salt thereof.

In certain embodiments, the present invention provides a compound of formula I, wherein LBM is KEAP1 binding moiety as recited in Lu et al., Euro. J. Med. Chem., 2018, 146:251-9, thereby forming a compound of formula I-o-10:

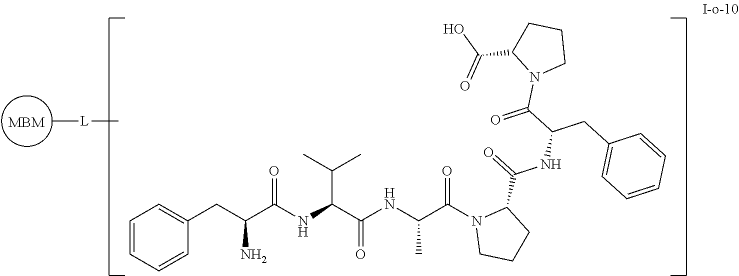

or a pharmaceutically acceptable salt thereof, wherein L and MBM are as defined above and described in embodiments herein.

In certain embodiments, the present invention provides a compound of formula I, wherein LBM is KEAP1-NRF2 binding moiety thereby forming a compound of formula I-o-11 or I-o-12:

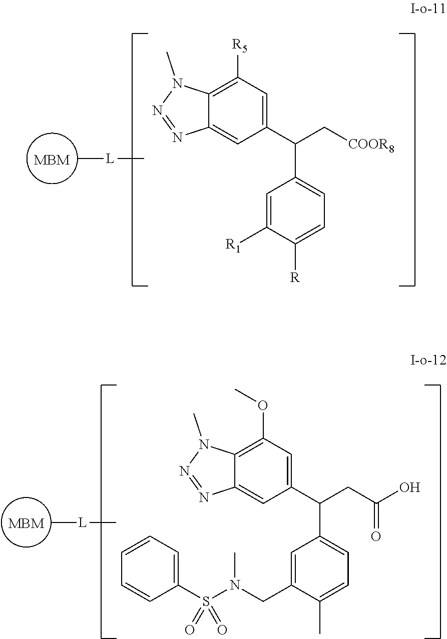

or a pharmaceutically acceptable salt thereof, wherein L and MBM are as defined above and described in embodiments herein, wherein each of the variables R, R₁, R₅, and R₈ is as described and defined in WO 2020/018788, the entirety of each of which is herein incorporated by reference.

In certain embodiments, the present invention provides a compound of formula I, wherein LBM is KEAP1-NRF2 binding moiety as recited in Tong et al., “Targeted Protein Degradation via a Covalent Reversible Degrader Based on Bardoxolone”, ChemRxiv 2020, thereby forming a compound of formula I-o-13 or I-o-14:

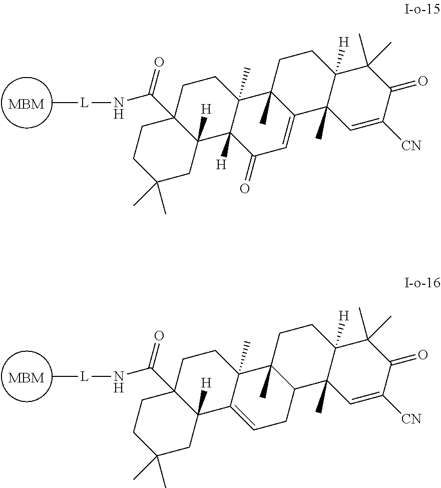

or a pharmaceutically acceptable salt thereof, wherein L and MBM are as defined above and described in embodiments herein.

Lysine Mimetic

In some embodiments, DIM is LBM as described above and herein. In some embodiments, DIM is a lysine mimetic. In some embodiments, the covalent attachment of ubiquitin to MK2 protein is achieved through the action of a lysine mimetic. In some embodiments, upon the binding of a compound of formula I to MK2, the DIM moiety that mimics a lysine undergoes ubiquitination thereby marking MK2 for degradation via the Ubiquitin-Proteasome Pathway (UPP).

In some embodiments, DIM is

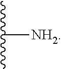 [SMILES: *N]

In some embodiments, DIM is

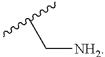 [SMILES: *CN]

In some embodiments, DIM is

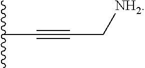 [SMILES: *C#CCN]

In some embodiments, DIM is selected from those depicted in Table 1, below.

In some embodiments, the present invention provides the compound of formula I as a compound of formula I-p-1:

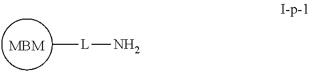

or a pharmaceutically acceptable salt thereof, wherein each of MBM and L is as defined above and described in embodiments herein, both singly and in combination.

In some embodiments, the present invention provides the compound of formula I as a compound of formula I-p-2:

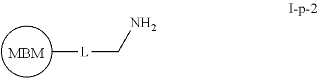

or a pharmaceutically acceptable salt thereof, wherein each of MBM and L is as defined above and described in embodiments herein, both singly and in combination.

In some embodiments, the present invention provides the compound of formula I as a compound of formula I-p-3:

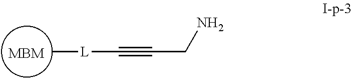

or a pharmaceutically acceptable salt thereof, wherein each of MBM and L is as defined above and described in embodiments herein, both singly and in combination.

In certain embodiments, the present invention provides a compound of formula I, wherein DIM is a lysine mimetic

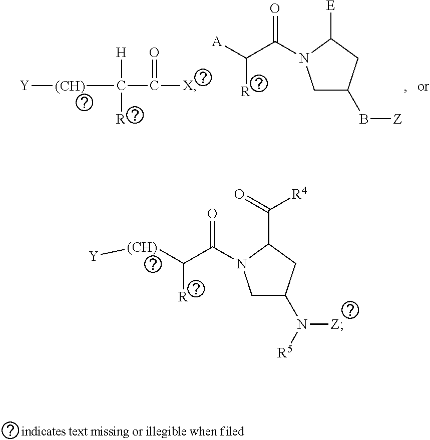

thereby forming a compound of Formulae I-q-1, I-q-2, or I-q-3, respectively:

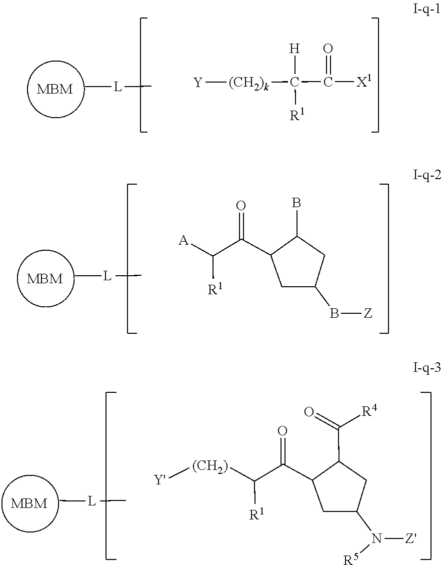

or a pharmaceutically acceptable salt thereof, wherein L and MBM are as defined above and described in embodiments herein, and wherein each of the variables R¹, R⁴, R⁵, A, B, E, Y, Y′, Z, Z′, and k are as defined and described in U.S. Pat. No. 7,622,496, the entirety of each of which is herein incorporated by reference.

Hydrogen Atom

In some embodiments, DIM is a hydrogen atom. In some embodiments, the covalent attachment of ubiquitin to MK2 protein is achieved through a provided compound wherein DIM is a hydrogen atom. In some embodiments, upon the binding of a compound of formula I to MK2, the DIM moiety being hydrogen effectuates ubiquitination thereby marking MK2 for degradation via the Ubiquitin-Proteasome Pathway (UPP).

In some embodiments, DIM is selected from those depicted in Table 1, below.

In some embodiments, the present invention provides the compound of formula I wherein DIM is a hydrogen atom, thereby forming a compound of formula I-r:

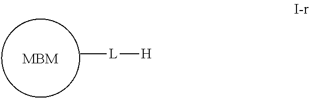

or a pharmaceutically acceptable salt thereof, wherein each of MBM and L is as defined above and described in embodiments herein, both singly and in combination.

Linker (L)

As defined above and described herein, L is a bivalent moiety that connects to MBM to DIM.

In some embodiments, L is a bivalent moiety that connects MBM to DIM. In some embodiments, L is a bivalent moiety that connects MBM to LBM. In some embodiments, L is a bivalent moiety that connects MBM to a lysine mimetic.

In some embodiments, L is a covalent bond or a bivalent, saturated or partially unsaturated, straight or branched C₁₋₅₀ hydrocarbon chain, wherein 0-10 methylene units of L are independently replaced by —C(D)(H)—, —C(D)₂-, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

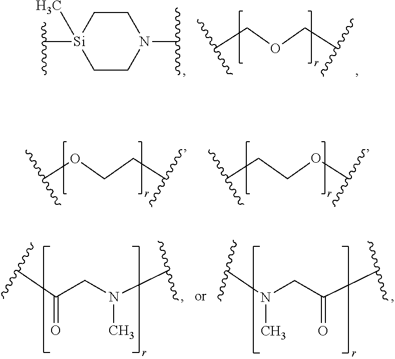

wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur; and
r is 0, 1, 2, 3, 4, 5, 6, 7, 8, 9,or 10.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 4-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 4-11 membered saturated or partially unsaturated spiro heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, -Cy- is

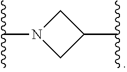 [SMILES: *C1CN(*)C1]

In some embodiments, -Cy- is

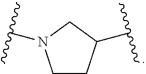 [SMILES: *C1CCN(*)C1]

In some embodiments, -Cy- is

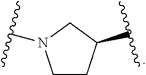 [SMILES: *[C@H]1CCN(*)C1]

In some embodiments, -Cy- is

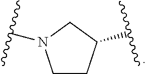 [SMILES: *[C@@H]1CCN(*)C1]

In some embodiments, -Cy- is

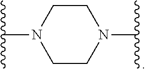 [SMILES: *N1CCN(*)CC1]

In some embodiments, -Cy- is

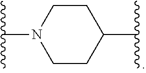 [SMILES: *C1CCN(*)CC1]

In some embodiments, -Cy- is

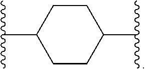 [SMILES: *C1CCC(*)CC1]

In some embodiments, -Cy- is

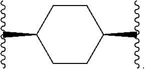 [SMILES: *[C@H]1CC[C@@H](*)CC1]

In some embodiments, -Cy- is

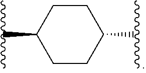 [SMILES: *[C@H]1CC[C@H](*)CC1]

In some embodiments, -Cy- is

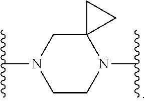 [SMILES: *N1CCN(*)C2(CC2)C1]

In some embodiments, -Cy- is

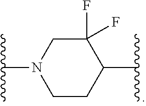 [SMILES: *C1CCN(*)CC1(F)F]

In some embodiments, -Cy- is

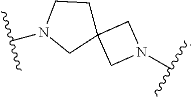 [SMILES: *N1CCC2(C1)CN(*)C2]

In some embodiments, -Cy- is

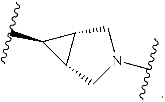 [SMILES: *[C@@H]1[C@H]2CN(*)C[C@@H]12]

In some embodiments, -Cy- is

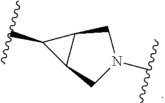 [SMILES: *[C@H]1[C@H]2CN(*)C[C@@H]12]

In some embodiments, -Cy- is

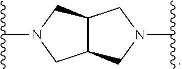 [SMILES: *N1C[C@H]2CN(*)C[C@H]2C1]

In some embodiments, -Cy- is

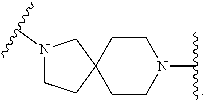 [SMILES: *N1CCC2(CC1)CCN(*)C2]

In some embodiments, -Cy- is

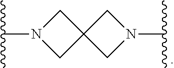 [SMILES: *N1CC2(C1)CN(*)C2]

In some embodiments, -Cy- is

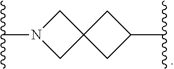 [SMILES: *C1CC2(C1)CN(*)C2]

In some embodiments, -Cy- is

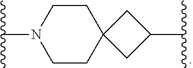 [SMILES: *C1CC2(CCN(*)CC2)C1]

In some embodiments, -Cy- is

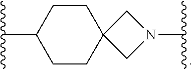 [SMILES: *C1CCC2(CC1)CN(*)C2]

In some embodiments, -Cy- is

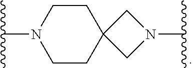 [SMILES: *N1CCC2(CC1)CN(*)C2]

In some embodiments, -Cy- is

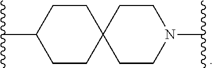 [SMILES: *C1CCC2(CC1)CCN(*)CC2]

In some embodiments, -Cy- is

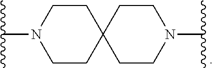 [SMILES: *N1CCC2(CC1)CCN(*)CC2]

In some embodiments, -Cy- is selected from those as depicted in the compounds of Table 1, below.

In some embodiments, L is —NR—(C₁₋₁₀ aliphatic)-. In some embodiments, L is —(C₁₋₁₀ aliphatic)-NR—(C₁₋₁₀aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀aliphatic)-. In some embodiments, L is —(C₁₋₁₀ aliphatic)-CONR—(CH₂CH₂O)₁₋₁₀CH₂CH₂—. In some embodiments, L is -Cy-CONR—(C₁₋₁₀ aliphatic)-. In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀aliphatic)-. In some embodiments, L is —(C₁₋₁₀ aliphatic)-NRCO—(CH₂CH₂O)₁₋₁₀CH₂CH₂—. In some embodiments, L is -Cy-NRCO—(C₁₋₁₀ aliphatic)-. In some embodiments, L is -Cy-(C₁₋₁₀ aliphatic)-NRCO—. In some embodiments, L is -Cy-(C₁₋₁₀ aliphatic)-NRCO—(C₁₋₁₀ aliphatic)-. In some embodiments, L is —(C₁₋₁₀ aliphatic)-Cy-NRCO—(C₁₋₁₀ aliphatic)-. In some embodiments, L is —(C₁₋₁₀ aliphatic)-Cy-(C₁₋₁₀ aliphatic)-NRCO—. In some embodiments, L is —(C₁₋₁₀ aliphatic)-Cy-(C₁₋₁₀ aliphatic)-NRCO—(C₁₋₁₀ aliphatic)-. In some embodiments, L is -Cy-(C₁₋₁₀ aliphatic)-Cy-NRCO—. In some embodiments, L is -Cy-(C₁₋₁₀ aliphatic)-NRCO-Cy-. In some embodiments, L is -Cy-(C₁₋₁₀ aliphatic)-Cy-NRCO—(C₁₋₁₀ aliphatic)-. In some embodiments, L is -Cy-(C₁₋₁₀ aliphatic)-NRCO-Cy-(C₁₋₁₀ aliphatic)-.

In some embodiments, L is —O—(C₁₋₁₀ aliphatic)-. In some embodiments, L is —(C₁₋₁₀ aliphatic)-O—(C₁₋₁₀aliphatic)-. In some embodiments, L is —(C₁₋₁₀ aliphatic)-O—(CH₂CH₂O)₁₋₁₀CH₂CH₂—. In some embodiments, L is -Cy-O—(C₁₋₁₀ aliphatic)-. In some embodiments, L is -Cy-(C₁₋₁₀ aliphatic)-O—. In some embodiments, L is -Cy-(C₁₋₁₀ aliphatic)-O—(C₁₋₁₀ aliphatic)-. In some embodiments, L is —(C₁₋₁₀ aliphatic)-Cy-O—(C₁₋₁₀ aliphatic)-. In some embodiments, L is —(C₁₋₁₀ aliphatic)-Cy-(C₁₋₁₀ aliphatic)-O—. In some embodiments, L is —(C₁₋₁₀ aliphatic)-Cy-(C₁₋₁₀ aliphatic)-O—(C₁₋₁₀ aliphatic)-. In some embodiments, L is —Cy-(C₁₋₁₀ aliphatic)-Cy-O—. In some embodiments, L is -Cy-(C₁₋₁₀ aliphatic)-O-Cy-. In some embodiments, L is -Cy-(C₁₋₁₀ aliphatic)-Cy-O—(C₁₋₁₀ aliphatic)-. In some embodiments, L is -Cy-(C₁₋₁₀ aliphatic)-O-Cy-(C₁₋₁₀ aliphatic)-.

In some embodiments, L is -Cy-(C₁₋₁₀ aliphatic)-. In some embodiments, L is —(C₁₋₁₀ aliphatic)-Cy-(C₁₋₁₀ aliphatic)-. In some embodiments, L is —(C₁₋₁₀ aliphatic)-Cy-(CH₂CH₂O)₁₋₁₀CH₂CH₂—. In some embodiments, L is -Cy-(C₁₋₁₀ aliphatic)-Cy-. In some embodiments, L is -Cy-(C₁₋₁₀ aliphatic)-Cy-(C₁₋₁₀ aliphatic)-. In some embodiments, L is -Cy-(C₁₋₁₀ aliphatic)-Cy-(C₁₋₁₀ aliphatic)-Cy-. In some embodiments, L is —(C₁₋₁₀ aliphatic)-Cy-(C₁₋₁₀ aliphatic)-Cy-(C₁₋₁₀ aliphatic)-.

In some embodiments, L is —NR—(CH₂)₁₋₁₀—. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is -Cy-NR—(CH₂)₁₋₁₀—. In some embodiments, L is -Cy-(CH₂)₁₋₁₀—NR—. In some embodiments, L is -Cy-(CH₂)₁₋₁₀—NR—(CH₂)₁₋₁₀—. In some embodiments, L is —(CH₂)₁₋₁₀-Cy-NR—(CH₂)₁₋₁₀—. In some embodiments, L is —(CH₂)₁₋₁₀-Cy-(CH₂)₁₋₁₀—NR—. In some embodiments, L is —(CH₂)₁₋₁₀-Cy-(CH₂)₁₋₁₀—NR—(CH₂)₁₋₁₀—. In some embodiments, L is -Cy-(CH₂)₁₋₁₀-Cy-NR—. In some embodiments, L is -Cy-(CH₂)₁₋₁₀—NR-Cy-. In some embodiments, L is -Cy-(CH₂)₁₋₁₀-Cy-NR—(CH₂)₁₋₁₀—. In some embodiments, L is -Cy-(CH₂)₁₋₁₀—NR—Cy-(CH₂)₁₋₁₀—.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(CH₂CH₂O)₁₋₁₀CH₂CH₂—. In some embodiments, L is -Cy-CONR—(CH₂)₁₋₁₀—. In some embodiments, L is -Cy-(CH₂)₁₋₁₀—CONR—. In some embodiments, L is -Cy-(CH₂)₁₋₁₀—CONR—(CH₂)₁₋₁₀—. In some embodiments, L is —(CH₂)₁₋₁₀-Cy-CONR—(CH₂)₁₋₁₀—. In some embodiments, L is —(CH₂)₁₋₁₀-Cy-(CH₂)₁₋₁₀—CONR—. In some embodiments, L is —(CH₂)₁₋₁₀-Cy-(CH₂)₁₋₁₀—CONR—(CH₂)₁₋₁₀—. In some embodiments, L is -Cy-(CH₂)₁₋₁₀-Cy-CONR—. In some embodiments, L is -Cy-(CH₂)₁₋₁₀—CONR-Cy-. In some embodiments, L is -Cy-(CH₂)₁₋₁₀-Cy-CONR—(CH₂)₁₋₁₀—. In some embodiments, L is -Cy-(CH₂)₁₋₁₀—CONR-Cy-(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, L is -Cy-NRCO—(CH₂)₁₋₁₀—. In some embodiments, L is -Cy-(CH₂)₁₋₁₀—NRCO—. In some embodiments, L is -Cy-(CH₂)₁₋₁₀—NRCO—(CH₂)₁₋₁₀—. In some embodiments, L is —(CH₂)₁₋₁₀-Cy-NRCO—(CH₂)₁₋₁₀—. In some embodiments, L is —(CH₂)₁₋₁₀-Cy-(CH₂)₁₋₁₀—NRCO—. In some embodiments, L is —(CH₂)₁₋₁₀-Cy-(CH₂)₁₋₁₀—NRCO—(CH₂)₁₋₁₀—. In some embodiments, L is -Cy-(CH₂)₁₋₁₀-Cy-NRCO—. In some embodiments, L is -Cy-(CH₂)₁₋₁₀—NRCO-Cy-. In some embodiments, L is -Cy-(CH₂)₁₋₁₀-Cy-NRCO—(CH₂)₁₋₁₀—. In some embodiments, L is -Cy-(CH₂)₁₋₁₀—NRCO-Cy-(CH₂)₁₋₁₀—.

In some embodiments, L is —O—(CH₂)₁₋₁₀—. In some embodiments, L is —(CH₂)₁₋₁₀—O—(CH₂)₁₋₁₀—. In some embodiments, L is —(CH₂)₁₋₁₀—O—(CH₂CH₂O)₁₋₁₀CH₂CH₂—. In some embodiments, L is -Cy-O—(CH₂)₁₋₁₀—. In some embodiments, L is -Cy-(CH₂)₁₋₁₀—O—. In some embodiments, L is -Cy-(CH₂)₁₋₁₀—O—(CH₂)₁₋₁₀—. In some embodiments, L is —(CH₂)₁₋₁₀-Cy-O—(CH₂)₁₋₁₀—. In some embodiments, L is —(CH₂)₁₋₁₀—Cy-(CH₂)₁₋₁₀—O—. In some embodiments, L is —(CH₂)₁₋₁₀-Cy-(CH₂)₁₋₁₀—O—(CH₂)₁₋₁₀—. In some embodiments, L is -Cy-(CH₂)₁₋₁₀-Cy-O—. In some embodiments, L is -Cy-(CH₂)₁₋₁₀—O-Cy-. In some embodiments, L is -Cy-(CH₂)₁₋₁₀-Cy-O—(CH₂)₁₋₁₀—. In some embodiments, L is -Cy-(CH₂)₁₋₁₀—O-Cy-(CH₂)₁₋₁₀—.

In some embodiments, L is -Cy-(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂CH₂O)₁₋₁₀CH₂CH₂—. In some embodiments, L is -Cy-(CH₂)₁₋₁₀-Cy-. In some embodiments, L is -Cy-(CH₂)₁₋₁₀-Cy-(CH₂)₁₋₁₀—. In some embodiments, L is -Cy-(CH₂)₁₋₁₀-Cy-(CH₂)₁₋₁₀-Cy-. In some embodiments, L is —(CH₂)₁₋₁₀-Cy-(CH₂)₁₋₁₀-Cy-(CH₂)₁₋₁₀—.

In some embodiments, L is

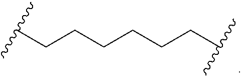 [SMILES: *CCCCCC*]

In some embodiments, L is

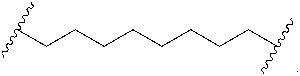 [SMILES: *CCCCCCCC*]

In some embodiments, L is

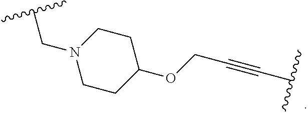 [SMILES: *C#CCOC1CCN(C*)CC1]

In some embodiments, L is

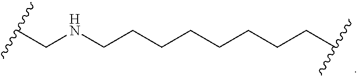 [SMILES: *CCCCCCCCNC*]

In some embodiments, L is

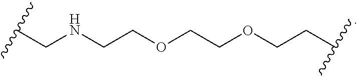 [SMILES: *CCOCCOCCNC*]

In some embodiments, L is

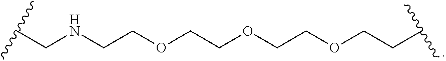 [SMILES: *CCOCCOCCOCCNC*]

In some embodiments, L is

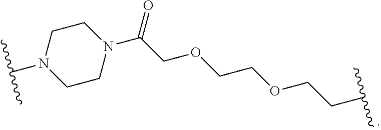 [SMILES: *CCOCCOCC(=O)N1CCN(*)CC1]

In some embodiments, L is

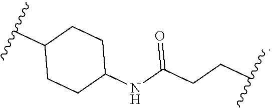 [SMILES: *CCC(=O)NC1CCC(*)CC1]

In some embodiments, L is

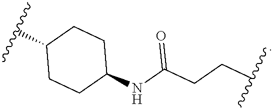 [SMILES: *CCC(=O)N[C@H]1CC[C@H](*)CC1]

In some embodiments, L is

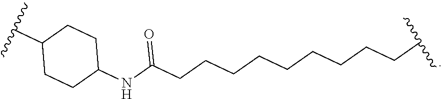 [SMILES: *CCCCCCCCCC(=O)NC1CCC(*)CC1]

In some embodiments, L is

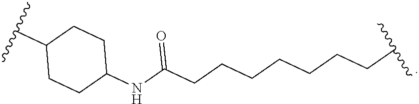 [SMILES: *CCCCCCCC(=O)NC1CCC(*)CC1]

In some embodiments, L is

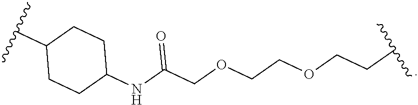 [SMILES: *CCOCCOCC(=O)NC1CCC(*)CC1]

In some embodiments, L is

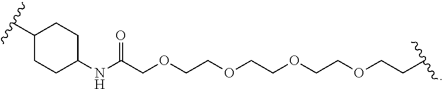 [SMILES: *CCOCCOCCOCCOCC(=O)NC1CCC(*)CC1]

In some embodiments, L is

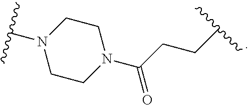 [SMILES: *CCC(=O)N1CCN(*)CC1]

In some embodiments, L is

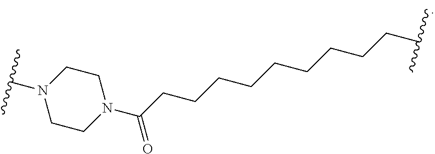 [SMILES: *CCCCCCCCCC(=O)N1CCN(*)CC1]

In some embodiments, L is

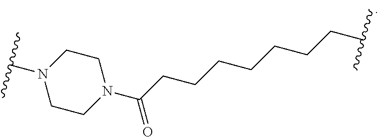 [SMILES: *CCCCCCCC(=O)N1CCN(*)CC1]

In some embodiments, L is

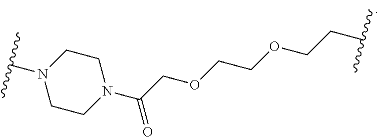 [SMILES: *CCOCCOCC(=O)N1CCN(*)CC1]

In some embodiments, L is

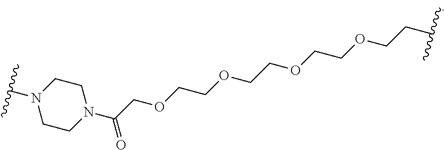 [SMILES: *CCOCCOCCOCCOCC(=O)N1CCN(*)CC1]

In some embodiments, L is

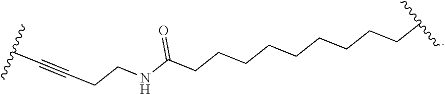 [SMILES: *C#CCCNC(=O)CCCCCCCCC*]

In some embodiments, L is

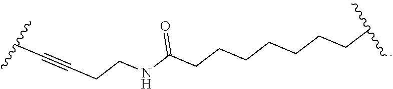 [SMILES: *C#CCCNC(=O)CCCCCCC*]

In some embodiments, L is

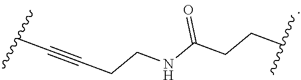 [SMILES: *C#CCCNC(=O)CC*]

In some embodiments, L is

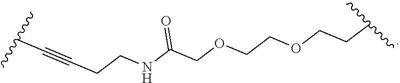 [SMILES: *C#CCCNC(=O)COCCOCC*]

In some embodiments, L is

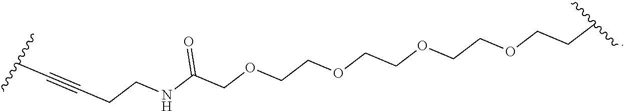 [SMILES: *C#CCCNC(=O)COCCOCCOCCOCC*]

In some embodiments, L is

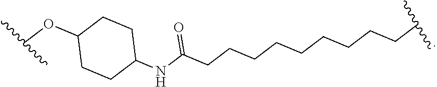 [SMILES: *CCCCCCCCCC(=O)NC1CCC(O*)CC1]

In some embodiments, L is

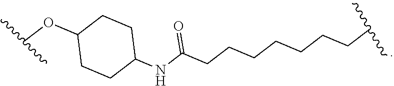 [SMILES: *CCCCCCCC(=O)NC1CCC(O*)CC1]

In some embodiments, L is

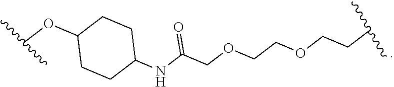 [SMILES: *CCOCCOCC(=O)NC1CCC(O*)CC1]

In some embodiments, L is

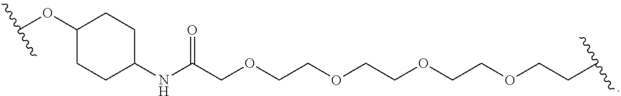 [SMILES: *CCOCCOCCOCCOCC(=O)NC1CCC(O*)CC1]

In some embodiments, L is

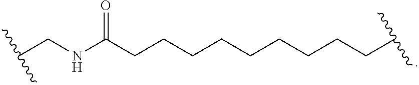 [SMILES: *CCCCCCCCCC(=O)NC*]

In some embodiments, L is

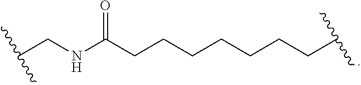 [SMILES: *CCCCCCCC(=O)NC*]

In some embodiments, L is

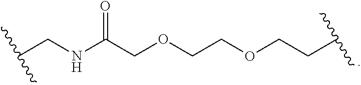 [SMILES: *CCOCCOCC(=O)NC*]

In some embodiments, L is

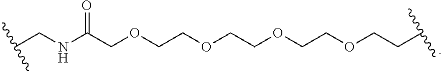 [SMILES: *CCOCCOCCOCCOCC(=O)NC*]

In some embodiments, L is

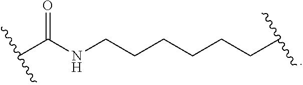 [SMILES: *CCCCCCNC(*)=O]

In some embodiments, L is

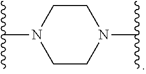 [SMILES: *N1CCN(*)CC1]

In some embodiments, L is

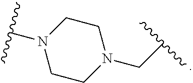 [SMILES: *CN1CCN(*)CC1]

In some embodiments, L is

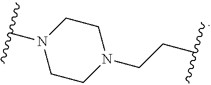 [SMILES: *CCN1CCN(*)CC1]

In some embodiments, L is

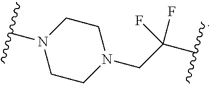 [SMILES: *N1CCN(CC(*)(F)F)CC1]

In some embodiments, L is

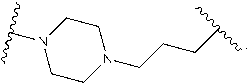 [SMILES: *CCCN1CCN(*)CC1]

In some embodiments, L is

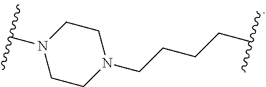 [SMILES: *CCCCN1CCN(*)CC1]

In some embodiments, L is

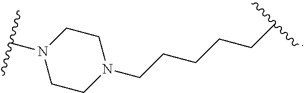 [SMILES: *CCCCCN1CCN(*)CC1]

In some embodiments, L is

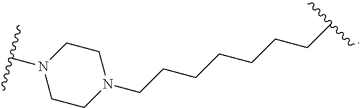 [SMILES: *CCCCCCCN1CCN(*)CC1]

In some embodiments, L is

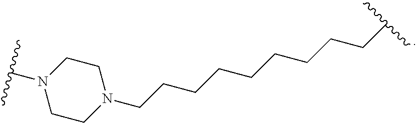 [SMILES: *CCCCCCCCCN1CCN(*)CC1]

In some embodiments, L is

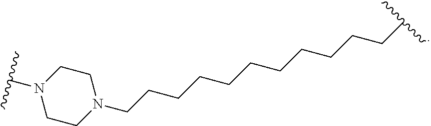 [SMILES: *CCCCCCCCCCCN1CCN(*)CC1]

In some embodiments, L is

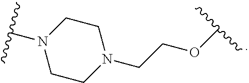 [SMILES: *OCCN1CCN(*)CC1]

In some embodiments, L is

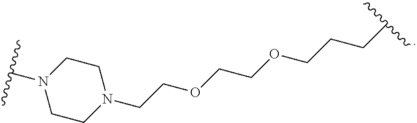 [SMILES: *CCCOCCOCCN1CCN(*)CC1]

In some embodiments, L is

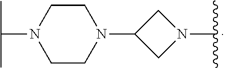 [SMILES: *N1CC(N2CCN(C(C)C)CC2)C1]

In some embodiments, L is

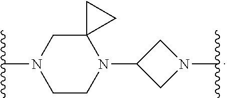 [SMILES: *N1CC(N2CCN(*)CC23CC3)C1]

In some embodiments, L is

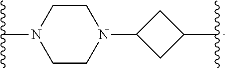 [SMILES: *C1CC(N2CCN(*)CC2)C1]

In some embodiments, L is

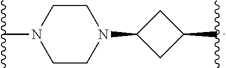 [SMILES: *N1CCN([C@H]2C[C@@H](*)C2)CC1]

In some embodiments, L is

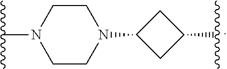 [SMILES: *N1CCN([C@H]2C[C@@H](*)C2)CC1]

In some embodiments, L is

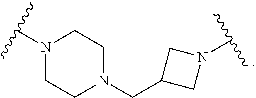 [SMILES: *N1CCN(CC2CN(*)C2)CC1]

In some embodiments, L is

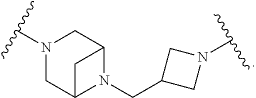 [SMILES: *N1CC(CN2C3CC2CN(*)C3)C1]

In some embodiments, L is

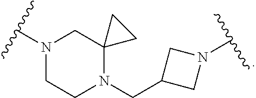 [SMILES: *N1CC(CN2CCN(*)CC23CC3)C1]

In some embodiments, L is

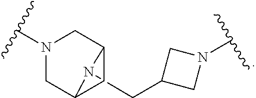 [SMILES: *N1CC(CN2C3CC2CN(*)C3)C1]

In some embodiments, L is

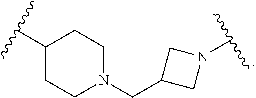 [SMILES: *C1CCN(CC2CN(*)C2)CC1]

In some embodiments, L is

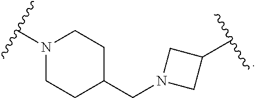 [SMILES: *C1CN(CC2CCN(*)CC2)C1]

In some embodiments, L is

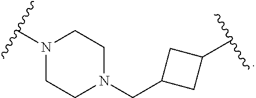 [SMILES: *C1CC(CN2CCN(*)CC2)C1]

In some embodiments, L is

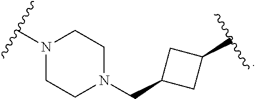 [SMILES: *N1CCN(C[C@H]2C[C@@H](*)C2)CC1]

In some embodiments, L is

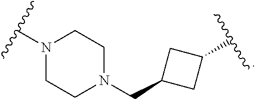 [SMILES: *N1CCN(C[C@H]2C[C@H](*)C2)CC1]

In some embodiments, L is

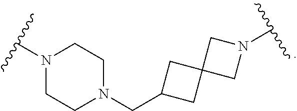 [SMILES: *N1CCN(CC2CC3(C2)CN(*)C3)CC1]

In some embodiments, L is

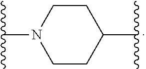 [SMILES: *C1CCN(*)CC1]

In some embodiments, L is

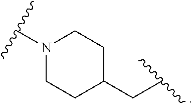 [SMILES: *CC1CCN(*)CC1]

In some embodiments, L is

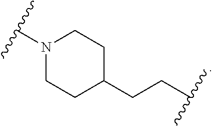 [SMILES: *CCC1CCN(*)CC1]

In some embodiments, L is

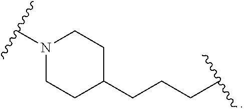 [SMILES: *CCCC1CCN(*)CC1]

In some embodiments, L is

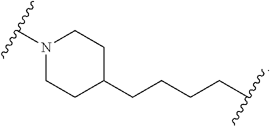 [SMILES: *CCCCC1CCN(*)CC1]

In some embodiments, L is

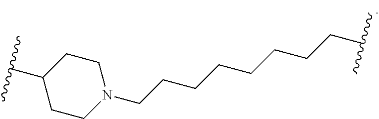 [SMILES: *CCCCCCCCN1CCC(*)CC1]

In some embodiments, L is

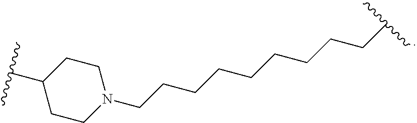 [SMILES: *CCCCCCCCCN1CCC(*)CC1]

In some embodiments, L is

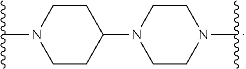 [SMILES: *N1CCC(N2CCN(*)CC2)CC1]

In some embodiments, L is

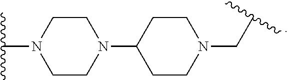 [SMILES: *CN1CCC(N2CCN(*)CC2)CC1]

In some embodiments, L is

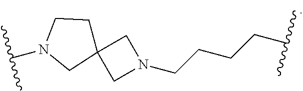 [SMILES: *CCCCN1CC2(CCN(*)C2)C1]

In some embodiments, L is

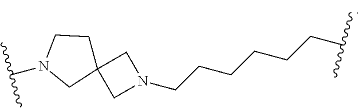 [SMILES: *CCCCCCN1CC2(CCN(*)C2)C1]

In some embodiments, L is

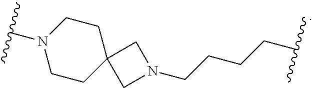 [SMILES: *CCCCN1CC2(CCN(*)CC2)C1]

In some embodiments, L is

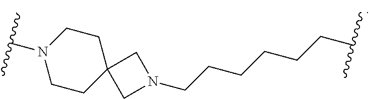 [SMILES: *CCCCCCN1CC2(CCN(*)CC2)C1]

In some embodiments, L is

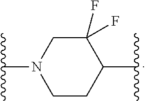 [SMILES: *C1CCN(*)CC1(F)F]

In some embodiments, L is

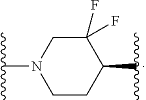 [SMILES: *[C@H]1CCN(*)CC1(F)F]

In some embodiments, L is

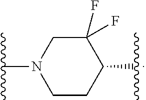 [SMILES: *[C@@H]1CCN(*)CC1(F)F]

In some embodiments, L is

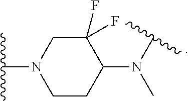 [SMILES: *N1CCC(N(*)C)C(F)(F)C1]

In some embodiments, L is

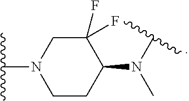 [SMILES: *N1CC[C@H](N(*)C)C(F)(F)C1]

In some embodiments, L is

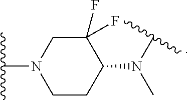 [SMILES: *N1CC[C@@H](N(*)C)C(F)(F)C1]

In some embodiments, L is

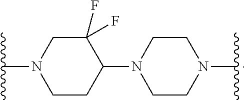 [SMILES: *N1CCN(C2CCN(*)CC2(F)F)CC1]

In some embodiments, L is

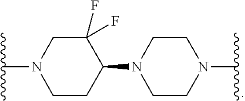 [SMILES: *N1CCN([C@H]2CCN(*)CC2(F)F)CC1]

In some embodiments, L is

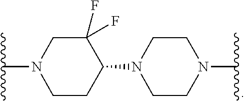 [SMILES: *N1CCN([C@@H]2CCN(*)CC2(F)F)CC1]

In some embodiments, L is

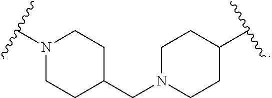 [SMILES: *C1CCN(CC2CCN(*)CC2)CC1]

In some embodiments, L is

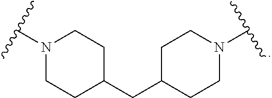 [SMILES: *N1CCC(CC2CCN(*)CC2)CC1]

In some embodiments, L is

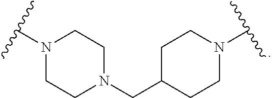 [SMILES: *N1CCC(CN2CCN(*)CC2)CC1]

In some embodiments, L is

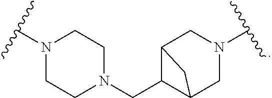 [SMILES: *N1CCN(CC2C3CC2CN(*)C3)CC1]

In some embodiments, L is

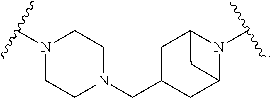 [SMILES: *N1CCN(CC2CC3CC(C2)N3*)CC1]

In some embodiments, L is

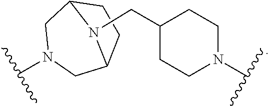 [SMILES: *N1CCC(CN2C3CCC2CN(*)C3)CC1]

In some embodiments, L is

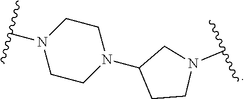 [SMILES: *N1CCN(C2CCN(*)C2)CC1]

In some embodiments, L is

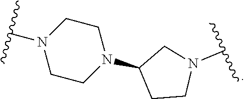 [SMILES: *N1CCN([C@@H]2CCN(*)C2)CC1]

In some embodiments, L is

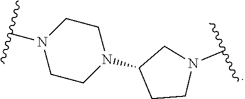 [SMILES: *N1CCN([C@H]2CCN(*)C2)CC1]

In some embodiments, L is

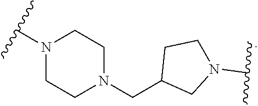 [SMILES: *N1CCN(CC2CCN(*)C2)CC1]

In some embodiments, L is

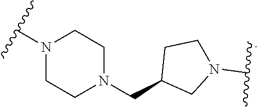 [SMILES: *N1CCN(C[C@H]2CCN(*)C2)CC1]

In some embodiments, L is

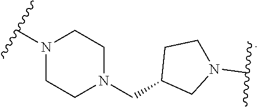 [SMILES: *N1CCN(C[C@@H]2CCN(*)C2)CC1]

In some embodiments, L is

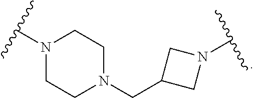 [SMILES: *N1CCN(CC2CN(*)C2)CC1]

In some embodiments, L is

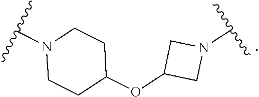 [SMILES: *N1CCC(OC2CN(*)C2)CC1]

In some embodiments, L is

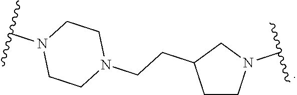 [SMILES: *N1CCN(CCC2CCN(*)C2)CC1]

In some embodiments, L is

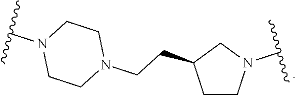 [SMILES: *N1CCN(CC[C@@H]2CCN(*)C2)CC1]

In some embodiments, L is

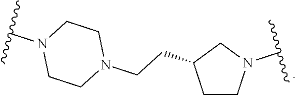 [SMILES: *N1CCN(CC[C@H]2CCN(*)C2)CC1]

In some embodiments, L is

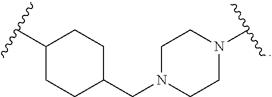 [SMILES: *C1CCC(CN2CCN(*)CC2)CC1]

In some embodiments, L is

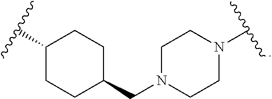 [SMILES: *N1CCN(C[C@H]2CC[C@H](*)CC2)CC1]

In some embodiments, L is

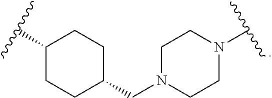 [SMILES: *N1CCN(C[C@H]2CC[C@@H](*)CC2)CC1]

In some embodiments, L is

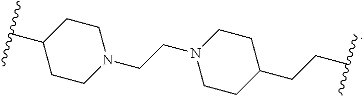 [SMILES: *CCC1CCN(CCN2CCC(*)CC2)CC1]

In some embodiments, L is

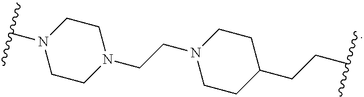 [SMILES: *CCC1CCN(CCN2CCN(*)CC2)CC1]

In some embodiments, L is

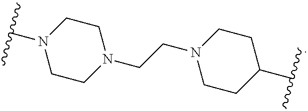 [SMILES: *C1CCN(CCN2CCN(*)CC2)CC1]

In some embodiments, L is

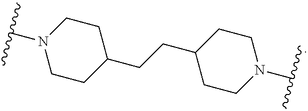 [SMILES: *N1CCC(CCC2CCN(*)CC2)CC1]

In some embodiments, L is

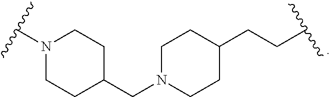 [SMILES: *CCC1CCN(CC2CCN(*)CC2)CC1]

In some embodiments, L is

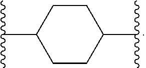 [SMILES: *C1CCC(*)CC1]

In some embodiments, L is

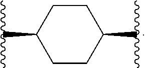 [SMILES: *[C@H]1CC[C@@H](*)CC1]

In some embodiments, L is

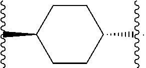 [SMILES: *[C@H]1CC[C@H](*)CC1]

In some embodiments, L is

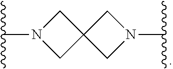 [SMILES: *N1CC2(C1)CN(*)C2]

In some embodiments, L is

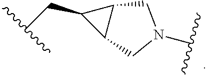 [SMILES: *C[C@@H]1[C@H]2CN(*)C[C@@H]12]

In some embodiments, L is

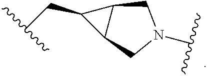 [SMILES: *C[C@H]1[C@H]2CN(*)C[C@@H]12]

In some embodiments, L is

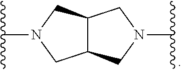 [SMILES: *N1C[C@H]2CN(*)C[C@H]2C1]

In some embodiments, L is

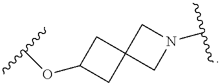 [SMILES: *OC1CC2(C1)CN(*)C2]

In some embodiments, L is

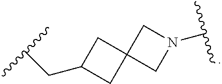 [SMILES: *CC1CC2(C1)CN(*)C2]

In some embodiments, L is

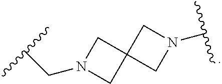 [SMILES: *CN1CC2(CN(*)C2)C1]

In some embodiments, L is

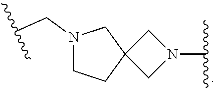 [SMILES: *CN1CCC2(CN(*)C2)C1]

In some embodiments, L is

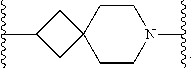 [SMILES: *C1CC2(CCN(*)CC2)C1]

In some embodiments, L is

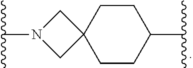 [SMILES: *C1CCC2(CC1)CN(*)C2]

In some embodiments, L is

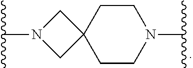 [SMILES: *N1CCC2(CC1)CN(*)C2]

In some embodiments, L is

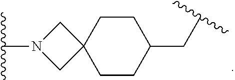 [SMILES: *CC1CCC2(CC1)CN(*)C2]

In some embodiments, L is

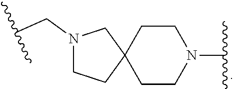 [SMILES: *CN1CCC2(CCN(*)CC2)C1]

In some embodiments, L is

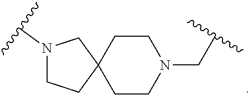 [SMILES: *CN1CCC2(CCN(*)C2)CC1]

In some embodiments, L is

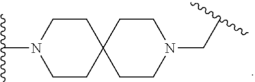 [SMILES: *CN1CCC2(CCN(*)CC2)CC1]

In some embodiments, L is

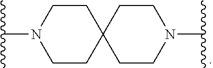 [SMILES: *N1CCC2(CC1)CCN(*)CC2]

In some embodiments, L is

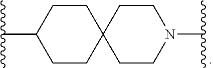 [SMILES: *C1CCC2(CC1)CCN(*)CC2]

In some embodiments, L is selected from those depicted in Table B, below.

In some embodiments, L is selected from those depicted in Table 1, below.

Without limitation, the point of attachment of L to MBM and DIM can be, for example when L is

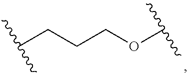 [SMILES: *CCCO*]

either

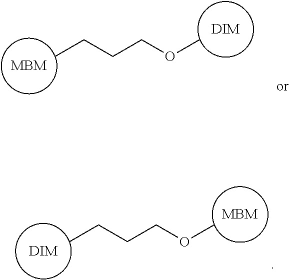

In some embodiments, a provided compound or pharmaceutically acceptable salt thereof, is selected from those wherein MBM is

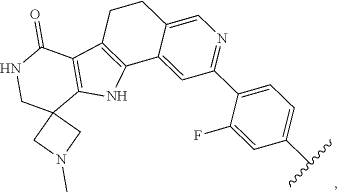 [SMILES: *c1ccc(-c2cc3c(cn2)CCc2c-3[nH]c3c2C(=O)NCC32CN(C)C2)c(F)c1]

LBM is selected from any of those in Table A below, and L is selected from any of those in Table B below.

In some embodiments, a provided compound or pharmaceutically acceptable salt thereof, is selected from those wherein MBM is

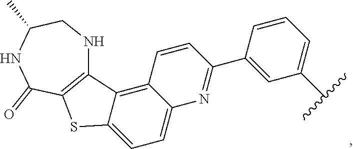 [SMILES: *c1cccc(-c2ccc3c(ccc4sc5c(c43)NC[C@@H](C)NC5=O)n2)c1]

LBM is selected from any of those in Table A below, and L is selected from any of those in Table B below.

In some embodiments, a provided compound or pharmaceutically acceptable salt thereof, is selected from those wherein MBM is

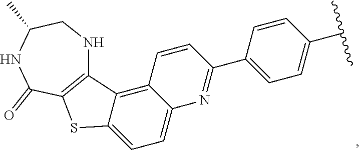 [SMILES: *c1ccc(-c2ccc3c(ccc4sc5c(c43)NC[C@@H](C)NC5=O)n2)cc1]

LBM is selected from any of those in Table A below, and L is selected from any of those in Table B below.

In some embodiments, a provided compound or pharmaceutically acceptable salt thereof, is selected from those wherein MBM is

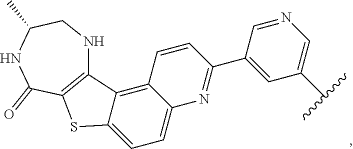 [SMILES: *c1cncc(-c2ccc3c(ccc4sc5c(c43)NC[C@@H](C)NC5=O)n2)c1]

LBM is selected from any of those in Table A below, and L is selected from any of those in Table B below.

In some embodiments, a provided compound or pharmaceutically acceptable salt thereof, is selected from those wherein MBM is

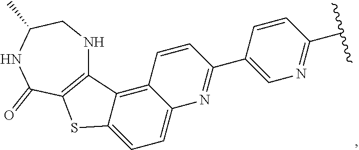 [SMILES: *c1ccc(-c2ccc3c(ccc4sc5c(c43)NC[C@@H](C)NC5=O)n2)cn1]

LBM is selected from any of those in Table A below, and L is selected from any of those in Table B below.

In some embodiments, a provided compound or pharmaceutically acceptable salt thereof, is selected from those wherein MBM is

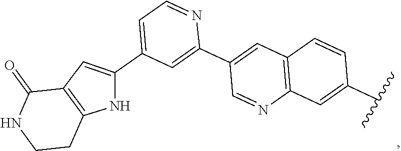 [SMILES: *c1ccc2cc(-c3cc(-c4cc5c([nH]4)CCNC5=O)ccn3)cnc2c1]

LBM is selected from any of those in Table A below, and L is selected from any of those in Table B below.

In some embodiments, a provided compound or pharmaceutically acceptable salt thereof, is selected from those wherein MBM is

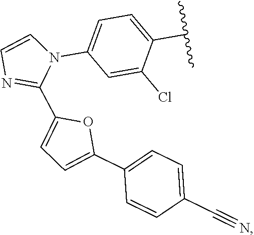 [SMILES: *c1ccc(-n2ccnc2-c2ccc(-c3ccc(C#N)cc3)o2)cc1Cl]

LBM is selected from any of those in Table A below, and L is selected from any of those in Table B below.

In some embodiments, a provided compound or pharmaceutically acceptable salt thereof, is selected from those wherein MBM is

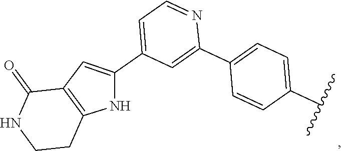 [SMILES: *c1ccc(-c2cc(-c3cc4c([nH]3)CCNC4=O)ccn2)cc1]

LBM is selected from any of those in Table A below, and L is selected from any of those in Table B below.

In some embodiments, a provided compound or pharmaceutically acceptable salt thereof, is selected from those wherein MBM is

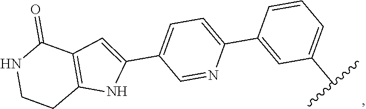 [SMILES: *c1cccc(-c2ccc(-c3cc4c([nH]3)CCNC4=O)cn2)c1]

LBM is selected from any of those in Table A below, and L is selected from any of those in Table B below.

In some embodiments, a provided compound or pharmaceutically acceptable salt thereof, is selected from those wherein MBM is

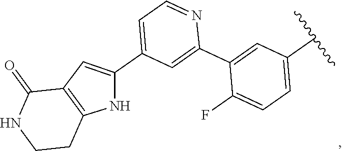 [SMILES: *c1ccc(F)c(-c2cc(-c3cc4c([nH]3)CCNC4=O)ccn2)c1]

LBM is selected from any of those in Table A below, and L is selected from any of those in Table B below.

In some embodiments, a provided compound or pharmaceutically acceptable salt thereof, is selected from those wherein MBM is

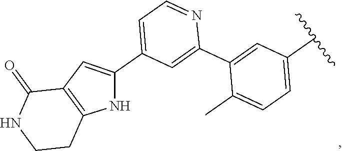 [SMILES: *c1ccc(C)c(-c2cc(-c3cc4c([nH]3)CCNC4=O)ccn2)c1]

LBM is selected from any of those in Table A below, and L is selected from any of those in Table B below.

In some embodiments, a provided compound or pharmaceutically acceptable salt thereof, is selected from those wherein MBM is

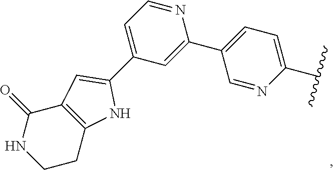 [SMILES: *c1ccc(-c2cc(-c3cc4c([nH]3)CCNC4=O)ccn2)cn1]

LBM is selected from any of those in Table A below, and L is selected from any of those in Table B below.

In some embodiments, a provided compound or pharmaceutically acceptable salt thereof, is selected from those wherein MBM is

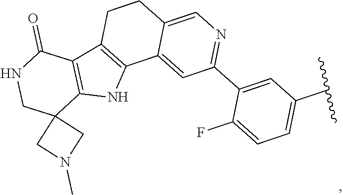 [SMILES: *c1ccc(F)c(-c2cc3c(cn2)CCc2c-3[nH]c3c2C(=O)NCC32CN(C)C2)c1]

LBM is selected from any of those in Table A below, and L is selected from any of those in Table B below.

In some embodiments, a provided compound or pharmaceutically acceptable salt thereof, is selected from those wherein MBM is

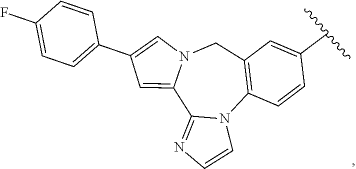 [SMILES: *c1ccc2c(c1)Cn1cc(-c3ccc(F)cc3)cc1-c1nccn1-2]

LBM is selected from any of those in Table A below, and L is selected from any of those in Table B below.

In some embodiments, a provided compound or pharmaceutically acceptable salt thereof, is selected from those wherein MBM is

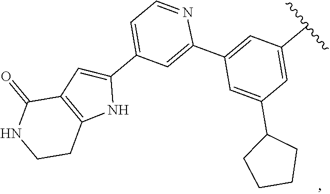 [SMILES: *c1cc(-c2cc(-c3cc4c([nH]3)CCNC4=O)ccn2)cc(C2CCCC2)c1]

LBM is selected from any of those in Table A below, and L is selected from any of those in Table B below.

In some embodiments, a provided compound or pharmaceutically acceptable salt thereof, is selected from those wherein MBM is

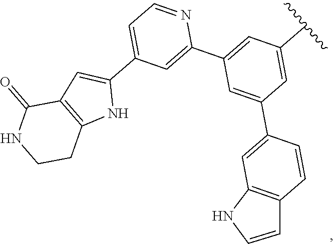 [SMILES: *c1cc(-c2ccc3cc[nH]c3c2)cc(-c2cc(-c3cc4c([nH]3)CCNC4=O)ccn2)c1]

LBM is selected from any of those in Table A below, and L is selected from any of those in Table B below.

In some embodiments, a provided compound or pharmaceutically acceptable salt thereof, is selected from those wherein MBM is

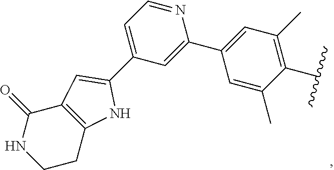 [SMILES: *c1c(C)cc(-c2cc(-c3cc4c([nH]3)CCNC4=O)ccn2)cc1C]

LBM is selected from any of those in Table A below, and L is selected from any of those in Table B below.

In some embodiments, a provided compound or pharmaceutically acceptable salt thereof, is selected from those wherein MBM is

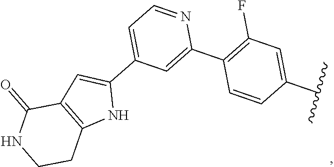 [SMILES: *c1ccc(-c2cc(-c3cc4c([nH]3)CCNC4=O)ccn2)c(F)c1]

LBM is selected from any of those in Table A below, and L is selected from any of those in Table B below.

In some embodiments, a provided compound or pharmaceutically acceptable salt thereof, is selected from those wherein MBM is

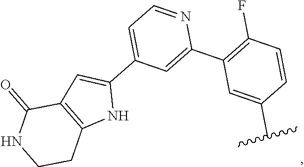 [SMILES: *c1ccc(F)c(-c2cc(-c3cc4c([nH]3)CCNC4=O)ccn2)c1]

LBM is selected from any of those in Table A below, and L is selected from any of those in Table B below.

In some embodiments, a provided compound or pharmaceutically acceptable salt thereof, is selected from those wherein MBM is

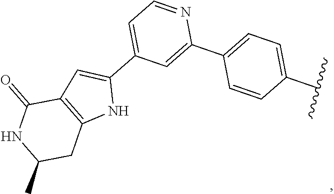 [SMILES: *c1ccc(-c2cc(-c3cc4c([nH]3)C[C@@H](C)NC4=O)ccn2)cc1]

LBM is selected from any of those in Table A below, and L is selected from any of those in Table B below.

In some embodiments, a provided compound or pharmaceutically acceptable salt thereof, is selected from those wherein MBM is

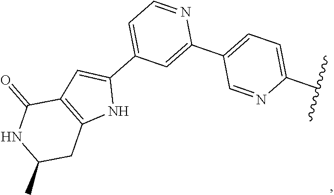 [SMILES: *c1ccc(-c2cc(-c3cc4c([nH]3)C[C@@H](C)NC4=O)ccn2)cn1]

LBM is selected from any of those in Table A below, and L is selected from any of those in Table B below.

In some embodiments, a provided compound or pharmaceutically acceptable salt thereof, is selected from those wherein MBM is

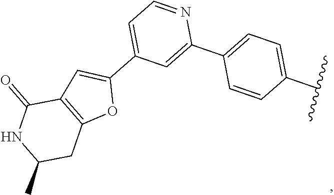 [SMILES: *c1ccc(-c2cc(-c3cc4c(o3)C[C@@H](C)NC4=O)ccn2)cc1]

LBM is selected from any of those in Table A below, and L is selected from any of those in Table B below.

In some embodiments, a provided compound or pharmaceutically acceptable salt thereof, is selected from those wherein MBM is

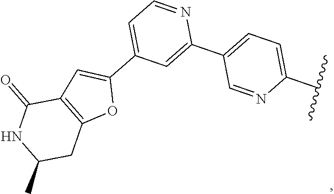 [SMILES: *c1ccc(-c2cc(-c3cc4c(o3)C[C@@H](C)NC4=O)ccn2)cn1]

LBM is selected from any of those in Table A below, and L is selected from any of those in Table B below.

In some embodiments, a provided compound or pharmaceutically acceptable salt thereof, is selected from those wherein MBM is

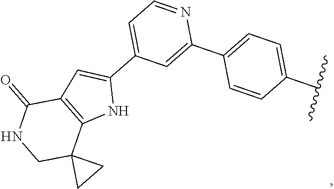 [SMILES: *c1ccc(-c2cc(-c3cc4c([nH]3)C3(CC3)CNC4=O)ccn2)cc1]

LBM is selected from any of those in Table A below, and L is selected from any of those in Table B below.

In some embodiments, a provided compound or pharmaceutically acceptable salt thereof, is selected from those wherein MBM is

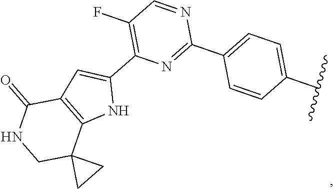 [SMILES: *c1ccc(-c2ncc(F)c(-c3cc4c([nH]3)C3(CC3)CNC4=O)n2)cc1]

LBM is selected from any of those in Table A below, and L is selected from any of those in Table B below.

In some embodiments, a provided compound or pharmaceutically acceptable salt thereof, is selected from those wherein MBM is

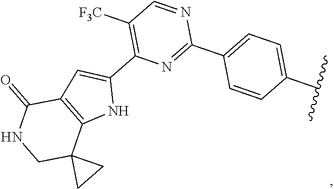 [SMILES: *c1ccc(-c2ncc(C(F)(F)F)c(-c3cc4c([nH]3)C3(CC3)CNC4=O)n2)cc1]

LBM is selected from any of those in Table A below, and L is selected from any of those in Table B below.

In some embodiments, a provided compound or pharmaceutically acceptable salt thereof, is selected from those wherein MBM is

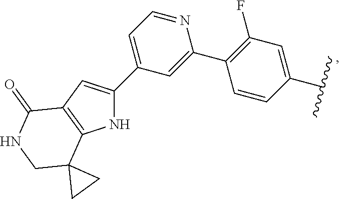 [SMILES: *c1ccc(-c2cc(-c3cc4c([nH]3)C3(CC3)CNC4=O)ccn2)c(F)c1]

LBM is selected from any of those in Table A below, and L is selected from any of those in Table B below.

In some embodiments, a provided compound or pharmaceutically acceptable salt thereof, is selected from those wherein MBM is

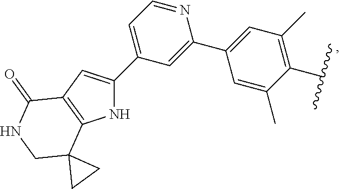 [SMILES: *c1c(C)cc(-c2cc(-c3cc4c([nH]3)C3(CC3)CNC4=O)ccn2)cc1C]

LBM is selected from any of those in Table A below, and L is selected from any of those in Table B below.

In some embodiments, a provided compound or pharmaceutically acceptable salt thereof, is selected from those wherein MBM is

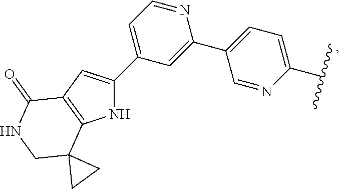 [SMILES: *c1ccc(-c2cc(-c3cc4c([nH]3)C3(CC3)CNC4=O)ccn2)cn1]

LBM is selected from any of those in Table A below, and L is selected from any of those in Table B below.

In some embodiments, a provided compound or pharmaceutically acceptable salt thereof, is selected from those wherein MBM is

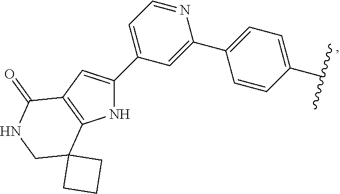 [SMILES: *c1ccc(-c2cc(-c3cc4c([nH]3)C3(CCC3)CNC4=O)ccn2)cc1]

LBM is selected from any of those in Table A below, and L is selected from any of those in Table B below.

In some embodiments, a provided compound or pharmaceutically acceptable salt thereof, is selected from those wherein MBM is

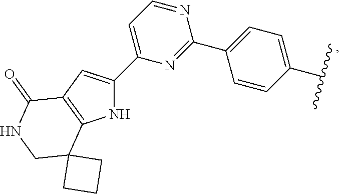 [SMILES: *c1ccc(-c2nccc(-c3cc4c([nH]3)C3(CCC3)CNC4=O)n2)cc1]

LBM is selected from any of those in Table A below, and L is selected from any of those in Table B below.

In some embodiments, a provided compound or pharmaceutically acceptable salt thereof, is selected from those wherein MBM is

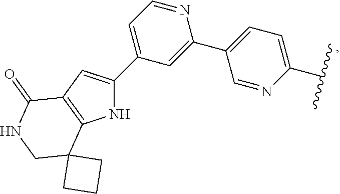 [SMILES: *c1ccc(-c2cc(-c3cc4c([nH]3)C3(CCC3)CNC4=O)ccn2)cn1]

LBM is selected from any of those in Table A below, and L is selected from any of those in Table B below.

In some embodiments, a provided compound or pharmaceutically acceptable salt thereof, is selected from those wherein MBM is

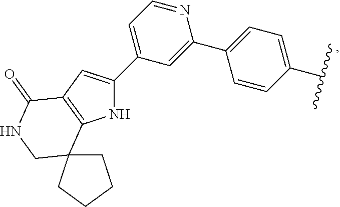 [SMILES: *c1ccc(-c2cc(-c3cc4c([nH]3)C3(CCCC3)CNC4=O)ccn2)cc1]

LBM is selected from any of those in Table A below, and L is selected from any of those in Table B below.

In some embodiments, a provided compound or pharmaceutically acceptable salt thereof, is selected from those wherein MBM is

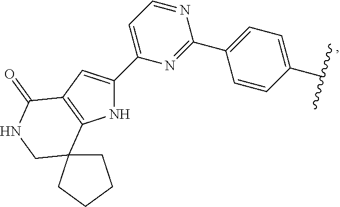 [SMILES: *c1ccc(-c2nccc(-c3cc4c([nH]3)C3(CCCC3)CNC4=O)n2)cc1]

LBM is selected from any of those in Table A below, and L is selected from any of those in Table B below.

In some embodiments, a provided compound or pharmaceutically acceptable salt thereof, is selected from those wherein MBM is

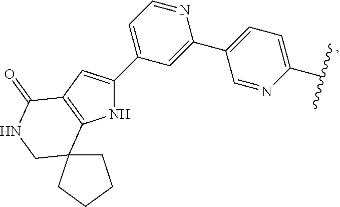 [SMILES: *c1ccc(-c2cc(-c3cc4c([nH]3)C3(CCCC3)CNC4=O)ccn2)cn1]

LBM is selected from any of those in Table A below, and L is selected from any of those in Table B below.

In some embodiments, a provided compound or pharmaceutically acceptable salt thereof, is selected from those wherein MBM is

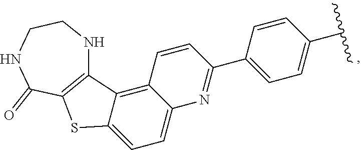 [SMILES: *c1ccc(-c2ccc3c(ccc4sc5c(c43)NCCNC5=O)n2)cc1]

LBM is selected from any of those in Table A below, and L is selected from any of those in Table B below.

In some embodiments, a provided compound or pharmaceutically acceptable salt thereof, is selected from those wherein MBM is

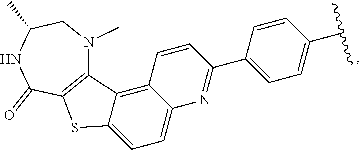 [SMILES: *c1ccc(-c2ccc3c(ccc4sc5c(c43)N(C)C[C@@H](C)NC5=O)n2)cc1]

LBM is selected from any of those in Table A below, and L is selected from any of those in Table B below.

In some embodiments, a provided compound or pharmaceutically acceptable salt thereof, is selected from those wherein MBM is

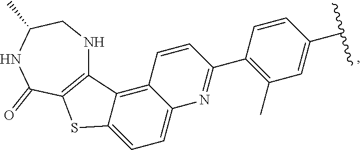 [SMILES: *c1ccc(-c2ccc3c(ccc4sc5c(c43)NC[C@@H](C)NC5=O)n2)c(C)c1]

LBM is selected from any of those in Table A below, and L is selected from any of those in Table B below.

In some embodiments, a provided compound or pharmaceutically acceptable salt thereof, is selected from those wherein MBM is

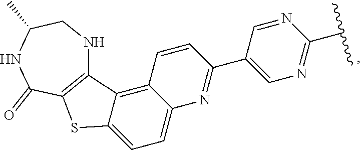 [SMILES: *c1ncc(-c2ccc3c(ccc4sc5c(c43)NC[C@@H](C)NC5=O)n2)cn1]

LBM is selected from any of those in Table A below, and L is selected from any of those in Table B below.

In some embodiments, a provided compound or pharmaceutically acceptable salt thereof, is selected from those wherein MBM is

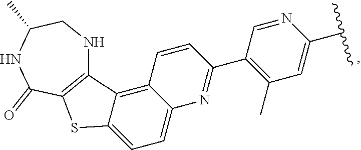 [SMILES: *c1cc(C)c(-c2ccc3c(ccc4sc5c(c43)NC[C@@H](C)NC5=O)n2)cn1]

LBM is selected from any of those in Table A below, and L is selected from any of those in Table B below.

In some embodiments, a provided compound or pharmaceutically acceptable salt thereof, is selected from those wherein MBM is

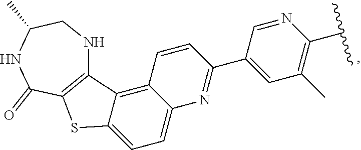 [SMILES: *c1ncc(-c2ccc3c(ccc4sc5c(c43)NC[C@@H](C)NC5=O)n2)cc1C]

LBM is selected from any of those in Table A below, and L is selected from any of those in Table B below.

In some embodiments, a provided compound or pharmaceutically acceptable salt thereof, is selected from those wherein MBM is

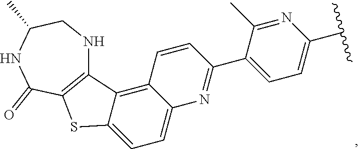 [SMILES: *c1ccc(-c2ccc3c(ccc4sc5c(c43)NC[C@@H](C)NC5=O)n2)c(C)n1]

LBM is selected from any of those in Table A below, and L is selected from any of those in Table B below.

In some embodiments, a provided compound or pharmaceutically acceptable salt thereof, is selected from those wherein MBM is

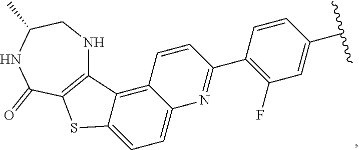 [SMILES: *c1ccc(-c2ccc3c(ccc4sc5c(c43)NC[C@@H](C)NC5=O)n2)c(F)c1]

LBM is selected from any of those in Table A below, and L is selected from any of those in Table B below.

In some embodiments, a provided compound or pharmaceutically acceptable salt thereof, is selected from those wherein MBM is

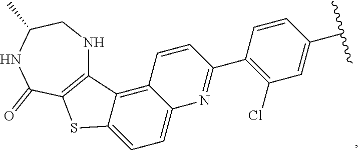 [SMILES: *c1ccc(-c2ccc3c(ccc4sc5c(c43)NC[C@@H](C)NC5=O)n2)c(Cl)c1]

LBM is selected from any of those in Table A below, and L is selected from any of those in Table B below.

In some embodiments, a provided compound or pharmaceutically acceptable salt thereof, is selected from those wherein MBM is

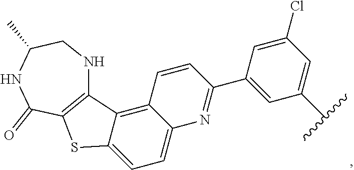 [SMILES: *c1cc(Cl)cc(-c2ccc3c(ccc4sc5c(c43)NC[C@@H](C)NC5=O)n2)c1]

LBM is selected from any of those in Table A below, and L is selected from any of those in Table B below.

In some embodiments, a provided compound or pharmaceutically acceptable salt thereof, is selected from those wherein MBM is

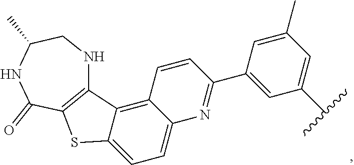 [SMILES: *c1cc(C)cc(-c2ccc3c(ccc4sc5c(c43)NC[C@@H](C)NC5=O)n2)c1]

LBM is selected from any of those in Table A below, and L is selected from any of those in Table B below.

In some embodiments, a provided compound or pharmaceutically acceptable salt thereof, is selected from those wherein MBM is

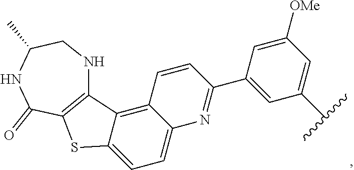 [SMILES: *c1cc(OC)cc(-c2ccc3c(ccc4sc5c(c43)NC[C@@H](C)NC5=O)n2)c1]

LBM is selected from any of those in Table A below, and L is selected from any of those in Table B below.

In some embodiments, a provided compound or pharmaceutically acceptable salt thereof, is selected from those wherein MBM is

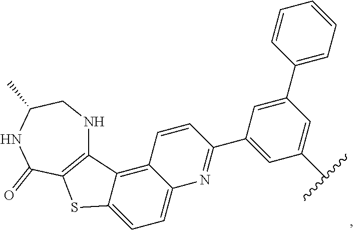 [SMILES: *c1cc(-c2ccccc2)cc(-c2ccc3c(ccc4sc5c(c43)NC[C@@H](C)NC5=O)n2)c1]

LBM is selected from any of those in Table A below, and L is selected from any of those in Table B below.

In some embodiments, a provided compound or pharmaceutically acceptable salt thereof, is selected from those wherein MBM is

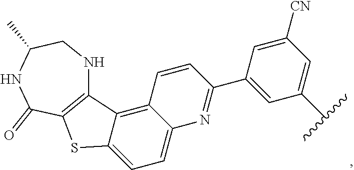 [SMILES: *c1cc(C#N)cc(-c2ccc3c(ccc4sc5c(c43)NC[C@@H](C)NC5=O)n2)c1]

LBM is selected from any of those in Table A below, and L is selected from any of those in Table B below.

In some embodiments, a provided compound or pharmaceutically acceptable salt thereof, is selected from those wherein MBM is

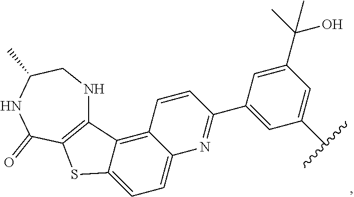 [SMILES: *c1cc(-c2ccc3c(ccc4sc5c(c43)NC[C@@H](C)NC5=O)n2)cc(C(C)(C)O)c1]

LBM is selected from any of those in Table A below, and L is selected from any of those in Table B below.

In some embodiments, a provided compound or pharmaceutically acceptable salt thereof, is selected from those wherein MBM is

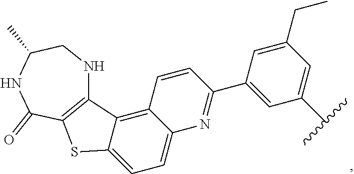 [SMILES: *c1cc(CC)cc(-c2ccc3c(ccc4sc5c(c43)NC[C@@H](C)NC5=O)n2)c1]

LBM is selected from any of those in Table A below, and L is selected from any of those in Table B below.

In some embodiments, a provided compound or pharmaceutically acceptable salt thereof, is selected from those wherein MBM is

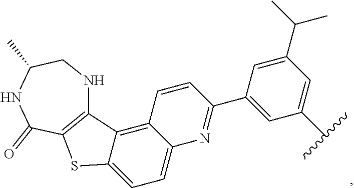 [SMILES: *c1cc(-c2ccc3c(ccc4sc5c(c43)NC[C@@H](C)NC5=O)n2)cc(C(C)C)c1]

LBM is selected from any of those in Table A below, and L is selected from any of those in Table B below.

In some embodiments, a provided compound or pharmaceutically acceptable salt thereof, is selected from those wherein MBM is

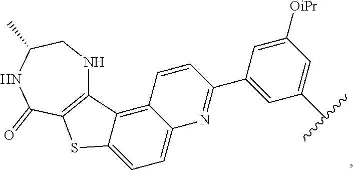 [SMILES: *c1cc(OC(C)C)cc(-c2ccc3c(ccc4sc5c(c43)NC[C@@H](C)NC5=O)n2)c1]

LBM is selected from any of those in Table A below, and L is selected from any of those in Table B below.

In some embodiments, a provided compound or pharmaceutically acceptable salt thereof, is selected from those wherein MBM is

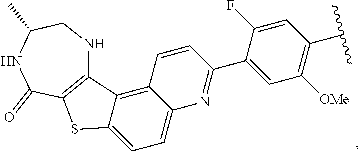 [SMILES: *c1cc(F)c(-c2ccc3c(ccc4sc5c(c43)NC[C@@H](C)NC5=O)n2)cc1OC]

LBM is selected from any of those in Table A below, and L is selected from any of those in Table B below.

In some embodiments, a provided compound or pharmaceutically acceptable salt thereof, is selected from those wherein MBM is

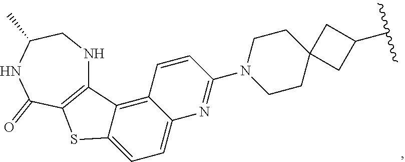 [SMILES: *C1CC2(CCN(c3ccc4c(ccc5sc6c(c54)NC[C@@H](C)NC6=O)n3)CC2)C1]

LBM is selected from any of those in Table A below, and L is selected from any of those in Table B below.

In some embodiments, a provided compound or pharmaceutically acceptable salt thereof, is selected from those wherein MBM is

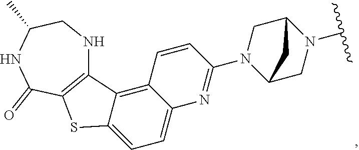 [SMILES: *N1C[C@H]2C[C@@H]1CN2c1ccc2c(ccc3sc4c(c32)NC[C@@H](C)NC4=O)n1]

LBM is selected from any of those in Table A below, and L is selected from any of those in Table B below.

In some embodiments, a provided compound or pharmaceutically acceptable salt thereof, is selected from those wherein MBM is

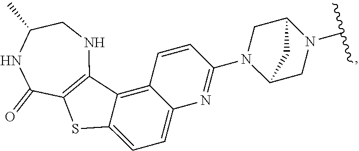

LBM is selected from any of those in Table A below, and L is selected from any of those in Table B below.

In some embodiments, a provided compound or pharmaceutically acceptable salt thereof, is selected from those wherein MBM is

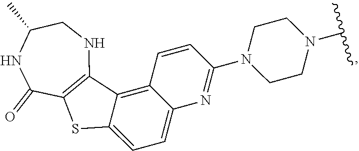 [SMILES: *N1CCN(c2ccc3c(ccc4sc5c(c43)NC[C@@H](C)NC5=O)n2)CC1]

LBM is selected from any of those in Table A below, and L is selected from any of those in Table B below.

In some embodiments, a provided compound or pharmaceutically acceptable salt thereof, is selected from those wherein MBM is

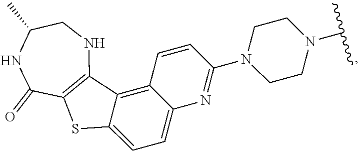 [SMILES: *N1CCN(c2ccc3c(ccc4sc5c(c43)NC[C@@H](C)NC5=O)n2)CC1]

LBM is selected from any of those in Table A below, and L is selected from any of those in Table B below.

In some embodiments, a provided compound or pharmaceutically acceptable salt thereof, is selected from those wherein MBM is

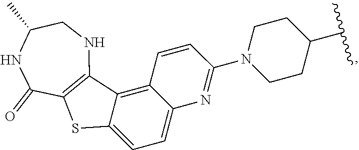 [SMILES: *C1CCN(c2ccc3c(ccc4sc5c(c43)NC[C@@H](C)NC5=O)n2)CC1]

LBM is selected from any of those in Table A below, and L is selected from any of those in Table B below.

In some embodiments, a provided compound or pharmaceutically acceptable salt thereof, is selected from those wherein MBM is

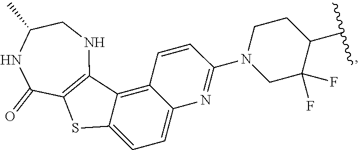 [SMILES: *C1CCN(c2ccc3c(ccc4sc5c(c43)NC[C@@H](C)NC5=O)n2)CC1(F)F]

LBM is selected from any of those in Table A below, and L is selected from any of those in Table B below.

In some embodiments, a provided compound or pharmaceutically acceptable salt thereof, is selected from those wherein MBM is

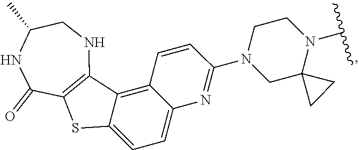 [SMILES: *N1CCN(c2ccc3c(ccc4sc5c(c43)NC[C@@H](C)NC5=O)n2)CC12CC2]

LBM is selected from any of those in Table A below, and L is selected from any of those in Table B below.

In some embodiments, a provided compound or pharmaceutically acceptable salt thereof, is selected from those wherein MBM is

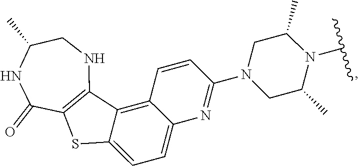 [SMILES: *N1[C@H](C)CN(c2ccc3c(ccc4sc5c(c43)NC[C@@H](C)NC5=O)n2)C[C@@H]1C]

LBM is selected from any of those in Table A below, and L is selected from any of those in Table B below.

In some embodiments, a provided compound or pharmaceutically acceptable salt thereof, is selected from those wherein MBM is

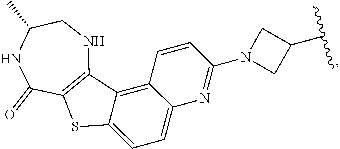 [SMILES: *C1CN(c2ccc3c(ccc4sc5c(c43)NC[C@@H](C)NC5=O)n2)C1]

LBM is selected from any of those in Table A below, and L is selected from any of those in Table B below.

In some embodiments, a provided compound or pharmaceutically acceptable salt thereof, is selected from those wherein MBM is

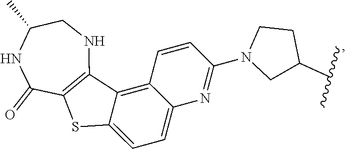 [SMILES: *C1CCN(c2ccc3c(ccc4sc5c(c43)NC[C@@H](C)NC5=O)n2)C1]

LBM is selected from any of those in Table A below, and L is selected from any of those in Table B below.

In some embodiments, a provided compound or pharmaceutically acceptable salt thereof, is selected from those wherein MBM is

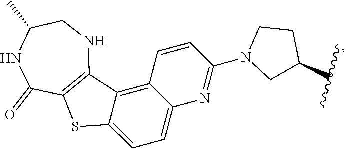 [SMILES: *[C@@H]1CCN(c2ccc3c(ccc4sc5c(c43)NC[C@@H](C)NC5=O)n2)C1]

LBM is selected from any of those in Table A below, and L is selected from any of those in Table B below.

In some embodiments, a provided compound or pharmaceutically acceptable salt thereof, is selected from those wherein MBM is

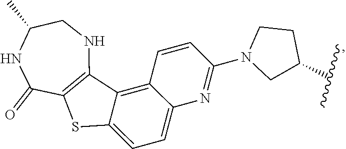 [SMILES: *[C@H]1CCN(c2ccc3c(ccc4sc5c(c43)NC[C@@H](C)NC5=O)n2)C1]

LBM is selected from any of those in Table A below, and L is selected from any of those in Table B below.

In some embodiments, a provided compound or pharmaceutically acceptable salt thereof, is selected from those wherein MBM is

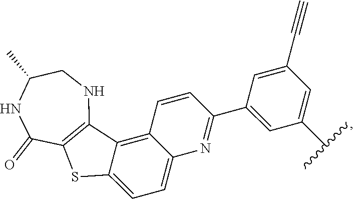 [SMILES: *c1cc(C#C)cc(-c2ccc3c(ccc4sc5c(c43)NC[C@@H](C)NC5=O)n2)c1]

LBM is selected from any of those in Table A below, and L is selected from any of those in Table B below.

In some embodiments, a provided compound or pharmaceutically acceptable salt thereof, is selected from those wherein MBM is

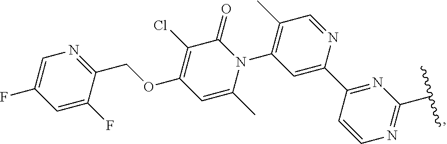 [SMILES: *c1nccc(-c2cc(-n3c(C)cc(OCc4ncc(F)cc4F)c(Cl)c3=O)c(C)cn2)n1]

LBM is selected from any of those in Table A below, and L is selected from any of those in Table B below.

In some embodiments, a provided compound or pharmaceutically acceptable salt thereof, is selected from those wherein MBM is

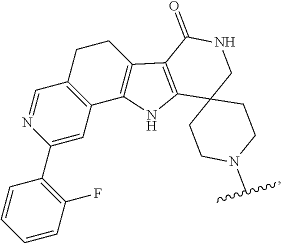 [SMILES: *N1CCC2(CC1)CNC(=O)c1c2[nH]c2c1CCc1cnc(-c3ccccc3F)cc1-2]

LBM is selected from any of those in Table A below, and L is selected from any of those in Table B below.

In some embodiments, a provided compound or pharmaceutically acceptable salt thereof, is selected from those wherein MBM is

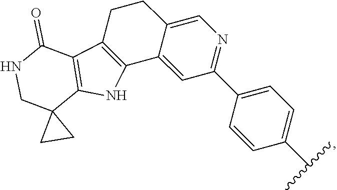 [SMILES: *c1ccc(-c2cc3c(cn2)CCc2c-3[nH]c3c2C(=O)NCC32CC2)cc1]

LBM is selected from any of those in Table A below, and L is selected from any of those in Table B below.

In some embodiments, a provided compound or pharmaceutically acceptable salt thereof, is selected from those wherein MBM is

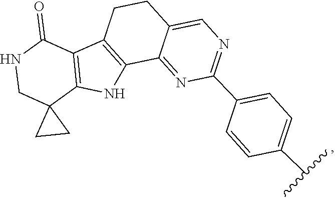 [SMILES: *c1ccc(-c2ncc3c(n2)-c2[nH]c4c(c2CC3)C(=O)NCC42CC2)cc1]

LBM is selected from any of those in Table A below, and L is selected from any of those in Table B below.

In some embodiments, a provided compound or pharmaceutically acceptable salt thereof, is selected from those wherein MBM is

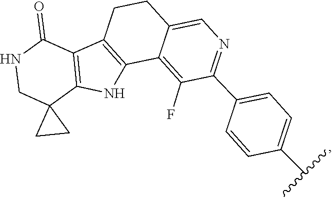 [SMILES: *c1ccc(-c2ncc3c(c2F)-c2[nH]c4c(c2CC3)C(=O)NCC42CC2)cc1]

LBM is selected from any of those in Table A below, and L is selected from any of those in Table B below.

In some embodiments, a provided compound or pharmaceutically acceptable salt thereof, is selected from those wherein MBM is

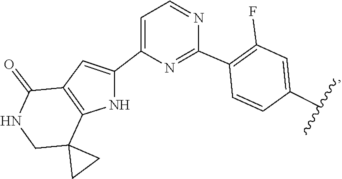 [SMILES: *c1ccc(-c2nccc(-c3cc4c([nH]3)C3(CC3)CNC4=O)n2)c(F)c1]

LBM is selected from any of those in Table A below, and L is selected from any of those in Table B below.

In some embodiments, a provided compound or pharmaceutically acceptable salt thereof, is selected from those wherein MBM is

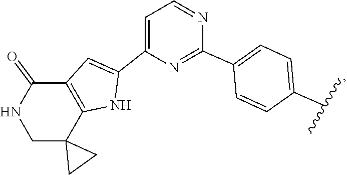 [SMILES: *c1ccc(-c2nccc(-c3cc4c([nH]3)C3(CC3)CNC4=O)n2)cc1]

LBM is selected from any of those in Table A below, and L is selected from any of those in Table B below.

In some embodiments, a provided compound or pharmaceutically acceptable salt thereof, is selected from those wherein MBM is

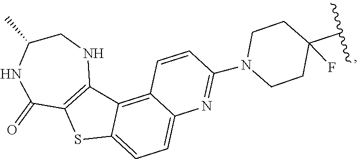 [SMILES: *C1(F)CCN(c2ccc3c(ccc4sc5c(c43)NC[C@@H](C)NC5=O)n2)CC1]

LBM is selected from any of those in Table A below, and L is selected from any of those in Table B below.

In some embodiments, a provided compound or pharmaceutically acceptable salt thereof, is selected from those wherein MBM is

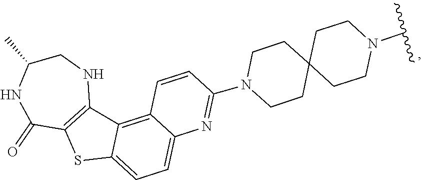 [SMILES: *N1CCC2(CC1)CCN(c1ccc3c(ccc4sc5c(c43)NC[C@@H](C)NC5=O)n1)CC2]

LBM is selected from any of those in Table A below, and L is selected from any of those in Table B below.

In some embodiments, a provided compound or pharmaceutically acceptable salt thereof, is selected from those wherein MBM is

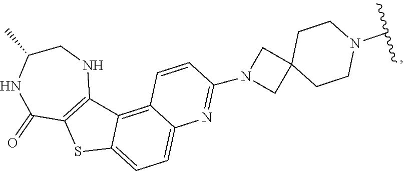 [SMILES: *N1CCC2(CC1)CN(c1ccc3c(ccc4sc5c(c43)NC[C@@H](C)NC5=O)n1)C2]

LBM is selected from any of those in Table A below, and L is selected from any of those in Table B below.

In some embodiments, a provided compound or pharmaceutically acceptable salt thereof, is selected from those wherein MBM is

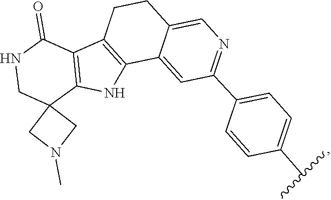 [SMILES: *c1ccc(-c2cc3c(cn2)CCc2c-3[nH]c3c2C(=O)NCC32CN(C)C2)cc1]

LBM is selected from any of those in Table A below, and L is selected from any of those in Table B below.

In some embodiments, a provided compound or pharmaceutically acceptable salt thereof, is selected from those wherein MBM is

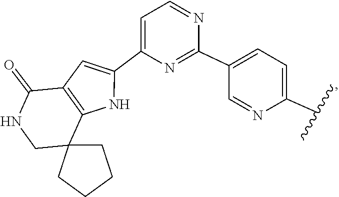 [SMILES: *c1ccc(-c2nccc(-c3cc4c([nH]3)C3(CCCC3)CNC4=O)n2)cn1]

LBM is selected from any of those in Table A below, and L is selected from any of those in Table B below.

In some embodiments, a provided compound or pharmaceutically acceptable salt thereof, is selected from those wherein MBM is

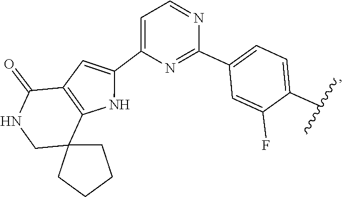 [SMILES: *c1ccc(-c2nccc(-c3cc4c([nH]3)C3(CCCC3)CNC4=O)n2)cc1F]

LBM is selected from any of those in Table A below, and L is selected from any of those in Table B below.

In some embodiments, a provided compound or pharmaceutically acceptable salt thereof, is selected from those wherein MBM is

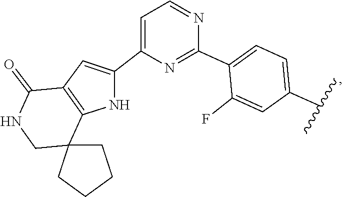 [SMILES: *c1ccc(-c2nccc(-c3cc4c([nH]3)C3(CCCC3)CNC4=O)n2)c(F)c1]

LBM is selected from any of those in Table A below, and L is selected from any of those in Table B below.

In some embodiments, a provided compound or pharmaceutically acceptable salt thereof, is selected from those wherein MBM is

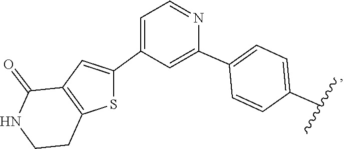 [SMILES: *c1ccc(-c2cc(-c3cc4c(s3)CCNC4=O)ccn2)cc1]

LBM is selected from any of those in Table A below, and L is selected from any of those in Table B below.

In some embodiments, a provided compound or pharmaceutically acceptable salt thereof, is selected from those wherein MBM is

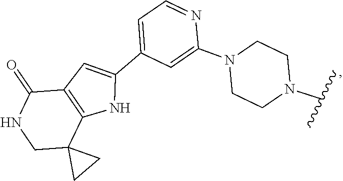 [SMILES: *N1CCN(c2cc(-c3cc4c([nH]3)C3(CC3)CNC4=O)ccn2)CC1]

LBM is selected from any of those in Table A below, and L is selected from any of those in Table B below.

In some embodiments, a provided compound or pharmaceutically acceptable salt thereof, is selected from those wherein MBM is

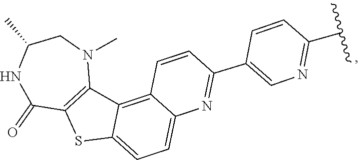 [SMILES: *c1ccc(-c2ccc3c(ccc4sc5c(c43)N(C)C[C@@H](C)NC5=O)n2)cn1]

LBM is selected from any of those in Table A below, and L is selected from any of those in Table B below.

In some embodiments, a provided compound or pharmaceutically acceptable salt thereof, is selected from those wherein MBM is

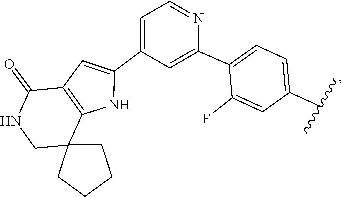 [SMILES: *c1ccc(-c2cc(-c3cc4c([nH]3)C3(CCCC3)CNC4=O)ccn2)c(F)c1]

LBM is selected from any of those in Table A below, and L is selected from any of those in Table B below.

In some embodiments, a provided compound or pharmaceutically acceptable salt thereof, is selected from those wherein MBM is

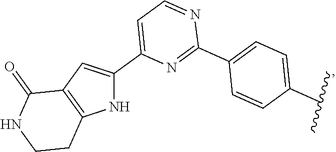 [SMILES: *c1ccc(-c2nccc(-c3cc4c([nH]3)CCNC4=O)n2)cc1]

LBM is selected from any of those in Table A below, and L is selected from any of those in Table B below.

In some embodiments, a provided compound or pharmaceutically acceptable salt thereof, is selected from those wherein MBM is

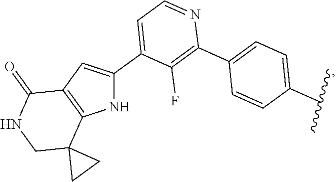 [SMILES: *c1ccc(-c2nccc(-c3cc4c([nH]3)C3(CC3)CNC4=O)c2F)cc1]

LBM is selected from any of those in Table A below, and L is selected from any of those in Table B below.

In some embodiments, a provided compound or pharmaceutically acceptable salt thereof, is selected from those wherein MBM is

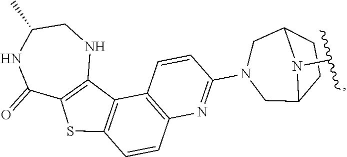 [SMILES: *N1C2CCC1CN(c1ccc3c(ccc4sc5c(c43)NC[C@@H](C)NC5=O)n1)C2]

LBM is selected from any of those in Table A below, and L is selected from any of those in Table B below.

In some embodiments, a provided compound or pharmaceutically acceptable salt thereof, is selected from those wherein MBM is

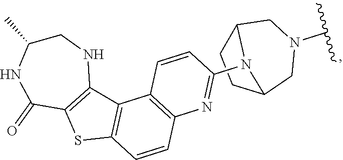 [SMILES: *N1CC2CCC(C1)N2c1ccc2c(ccc3sc4c(c32)NC[C@@H](C)NC4=O)n1]

LBM is selected from any of those in Table A below, and L is selected from any of those in Table B below.

In some embodiments, a provided compound or pharmaceutically acceptable salt thereof, is selected from those wherein MBM is

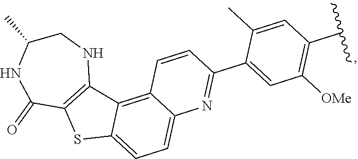 [SMILES: *c1cc(C)c(-c2ccc3c(ccc4sc5c(c43)NC[C@@H](C)NC5=O)n2)cc1OC]

LBM is selected from any of those in Table A below, and L is selected from any of those in Table B below.

In some embodiments, a provided compound or pharmaceutically acceptable salt thereof, is selected from those wherein MBM is

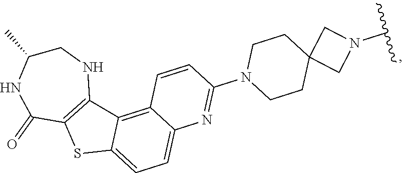 [SMILES: *N1CC2(CCN(c3ccc4c(ccc5sc6c(c54)NC[C@@H](C)NC6=O)n3)CC2)C1]

LBM is selected from any of those in Table A below, and L is selected from any of those in Table B below.

In some embodiments, a provided compound or pharmaceutically acceptable salt thereof, is selected from those wherein MBM is

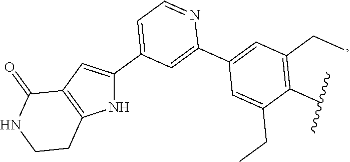 [SMILES: *c1c(CC)cc(-c2cc(-c3cc4c([nH]3)CCNC4=O)ccn2)cc1CC]

LBM is selected from any of those in Table A below, and L is selected from any of those in Table B below.

In some embodiments, a provided compound or pharmaceutically acceptable salt thereof, is selected from those wherein MBM is

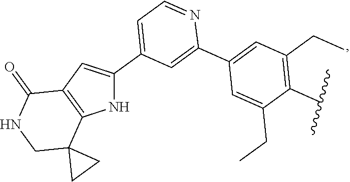 [SMILES: *c1c(CC)cc(-c2cc(-c3cc4c([nH]3)C3(CC3)CNC4=O)ccn2)cc1CC]

LBM is selected from any of those in Table A below, and L is selected from any of those in Table B below.

In some embodiments, a provided compound or pharmaceutically acceptable salt thereof, is selected from those wherein MBM is

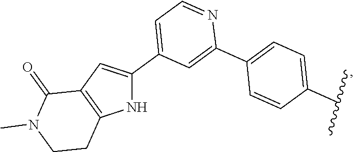 [SMILES: *c1ccc(-c2cc(-c3cc4c([nH]3)CCN(C)C4=O)ccn2)cc1]

LBM is selected from any of those in Table A below, and L is selected from any of those in Table B below.

In some embodiments, a provided compound or pharmaceutically acceptable salt thereof, is selected from those wherein MBM is

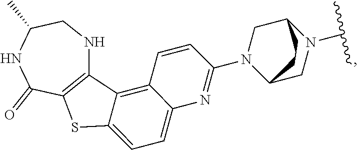 [SMILES: *N1C[C@H]2CC[C@@H]1CN2c1ccc2c(ccc3sc4c(c32)NC[C@@H](C)NC4=O)n1]

LBM is selected from any of those in Table A below, and L is selected from any of those in Table B below.

In some embodiments, a provided compound or pharmaceutically acceptable salt thereof, is selected from those wherein MBM is

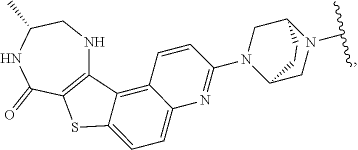 [SMILES: *N1C[C@@H]2CC[C@H]1CN2c1ccc2c(ccc3sc4c(c32)NC[C@@H](C)NC4=O)n1]

LBM is selected from any of those in Table A below, and L is selected from any of those in Table B below.

In some embodiments, a provided compound or pharmaceutically acceptable salt thereof, is selected from those wherein MBM is

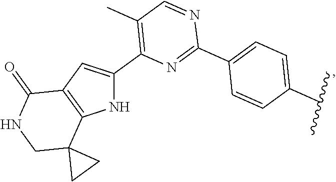 [SMILES: *c1ccc(-c2ncc(C)c(-c3cc4c([nH]3)C3(CC3)CNC4=O)n2)cc1]

LBM is selected from any of those in Table A below, and L is selected from any of those in Table B below.

In some embodiments, a provided compound or pharmaceutically acceptable salt thereof, is selected from those wherein MBM is

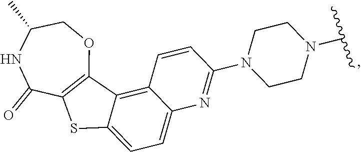 [SMILES: *N1CCN(c2ccc3c(ccc4sc5c(c43)OC[C@@H](C)NC5=O)n2)CC1]

LBM is selected from any of those in Table A below, and L is selected from any of those in Table B below.

In some embodiments, a provided compound or pharmaceutically acceptable salt thereof, is selected from those wherein MBM is

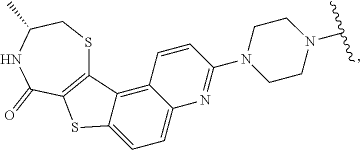 [SMILES: *N1CCN(c2ccc3c(ccc4sc5c(c43)SC[C@@H](C)NC5=O)n2)CC1]

LBM is selected from any of those in Table A below, and L is selected from any of those in Table B below.

In some embodiments, a provided compound or pharmaceutically acceptable salt thereof, is selected from those wherein MBM is

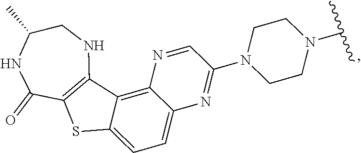 [SMILES: *N1CCN(c2cnc3c(ccc4sc5c(c43)NC[C@@H](C)NC5=O)n2)CC1]

LBM is selected from any of those in Table A below, and L is selected from any of those in Table B below.

TABLE A
Exemplified E3 Ligase Binding Moiety (LBM)

| | |
|---|---|
| 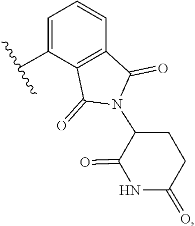 [SMILES: *c1cccc2c1C(=O)N(C1CCC(=O)NC1=O)C2=O] | (a) |
| 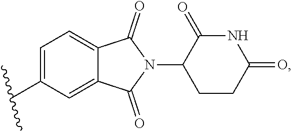 [SMILES: *c1ccc2c(c1)C(=O)N(C1CCC(=O)NC1=O)C2=O] | (b) |
| 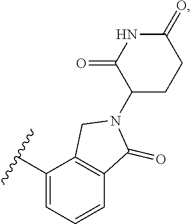 [SMILES: *c1cccc2c1CN(C1CCC(=O)NC1=O)C2=O] | (c) |
| 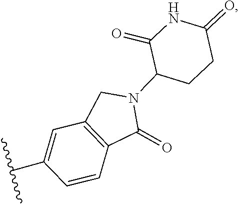 [SMILES: *c1ccc2c(c1)CN(C1CCC(=O)NC1=O)C2=O] | (d) |
| 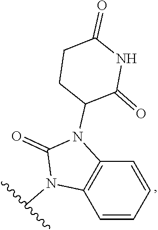 [SMILES: *n1c(=O)n(C2CCC(=O)NC2=O)c2ccccc21] | (e) |
| 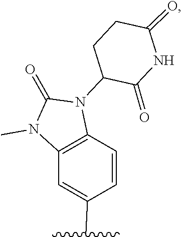 [SMILES: *c1ccc2c(c1)n(C)c(=O)n2C1CCC(=O)NC1=O] | (f) |
| 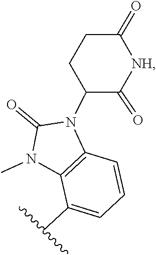 [SMILES: *c1cccc2c1n(C)c(=O)n2C1CCC(=O)NC1=O] | (g) |
| 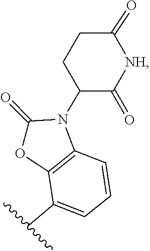 [SMILES: *c1cccc2c1oc(=O)n2C1CCC(=O)NC1=O] | (h) |
| 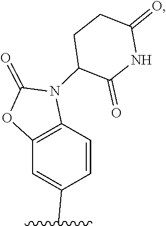 [SMILES: *c1ccc2c(c1)oc(=O)n2C1CCC(=O)NC1=O] | (i) |
| 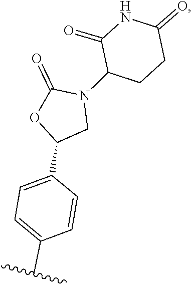 [SMILES: *c1ccc([C@H]2CN(C3CCC(=O)NC3=O)C(=O)O2)cc1] | (j) |
| 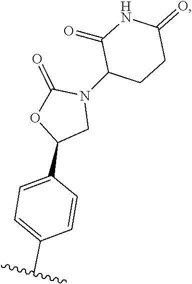 [SMILES: *c1ccc([C@@H]2CN(C3CCC(=O)NC3=O)C(=O)O2)cc1] | (k) |
| 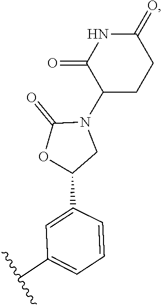 [SMILES: *c1cccc([C@H]2CN(C3CCC(=O)NC3=O)C(=O)O2)c1] | (l) |
| 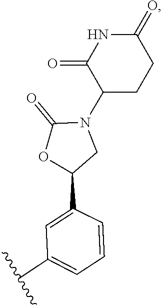 [SMILES: *c1cccc([C@@H]2CN(C3CCC(=O)NC3=O)C(=O)O2)c1] | (m) |
| 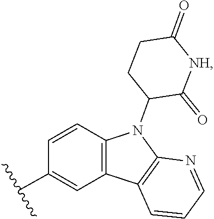 [SMILES: *c1ccc2c(c1)c1cccnc1n2C1CCC(=O)NC1=O] | (n) |
| 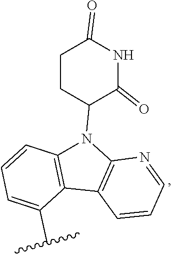 [SMILES: *c1cccc2c1c1cccnc1n2C1CCC(=O)NC1=O] | (o) |
| 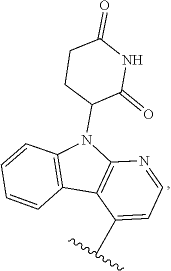 [SMILES: *c1ccnc2c1c1ccccc1n2C1CCC(=O)NC1=O] | (p) |
| 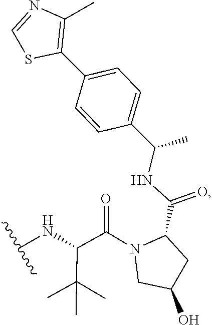 [SMILES: *N[C@H](C(=O)N1C[C@H](O)C[C@H]1C(=O)N[C@@H](C)c1ccc(-c2scnc2C)cc1)C(C)(C)C] | (q) |
| 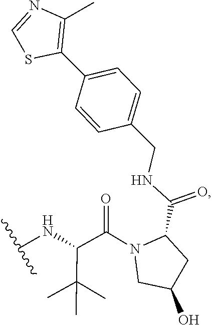 [SMILES: *N[C@H](C(=O)N1C[C@H](O)C[C@H]1C(=O)NCc1ccc(-c2scnc2C)cc1)C(C)(C)C] | (r) |
| 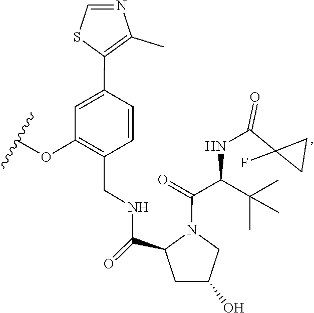 [SMILES: *Oc1cc(-c2scnc2C)ccc1CNC(=O)[C@@H]1C[C@@H](O)CN1C(=O)[C@@H](NC(=O)C1(F)CC1)C(C)(C)C] | (s) |
| 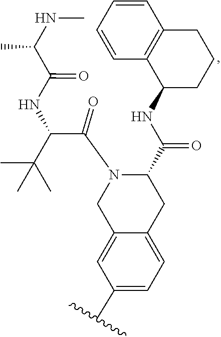 [SMILES: *c1ccc2c(c1)CN(C(=O)[C@@H](NC(=O)[C@H](C)NC)C(C)(C)C)[C@H](C(=O)N[C@@H]1CCCc3ccccc31)C2] | (t) |
| 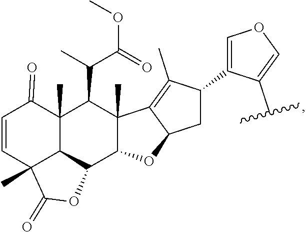 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45] | (u) |
| 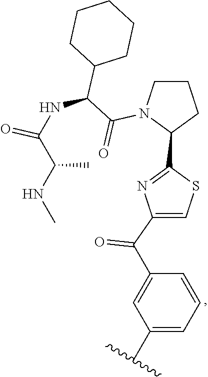 [SMILES: *c1cccc(C(=O)c2csc([C@@H]3CCCN3C(=O)[C@@H](NC(=O)[C@H](C)NC)C3CCCCC3)n2)c1] | (v) |
| 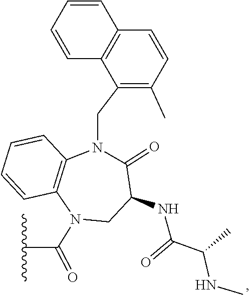 [SMILES: *C(=O)N1C[C@H](NC(=O)[C@H](C)NC)C(=O)N(Cc2c(C)ccc3ccccc23)c2ccccc21] | (w) |
| 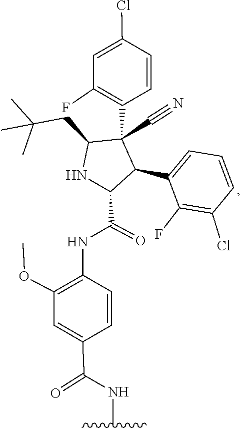 [SMILES: *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1] | (x) |
| 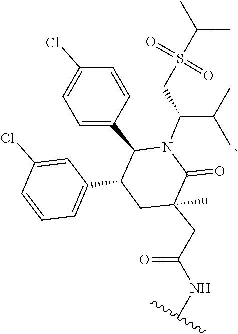 [SMILES: *NC(=O)C[C@@]1(C)C[C@H](c2cccc(Cl)c2)[C@@H](c2ccc(Cl)cc2)N([C@H](CS(=O)(=O)C(C)C)C(C)C)C1=O] | (y) |
| 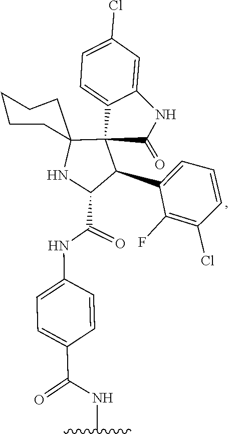 [SMILES: *NC(=O)c1ccc(NC(=O)[C@@H]2NC3(CCCCC3)[C@@]3(C(=O)Nc4cc(Cl)ccc43)[C@H]2c2cccc(Cl)c2F)cc1] | (z) |
| 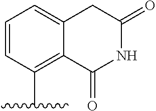 [SMILES: *c1cccc2c1C(=O)NC(=O)C2] | (aa) |
| 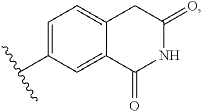 [SMILES: *c1ccc2c(c1)C(=O)NC(=O)C2] | (bb) |
| 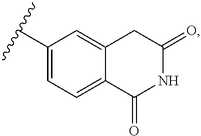 [SMILES: *c1ccc2c(c1)CC(=O)NC2=O] | (cc) |
| 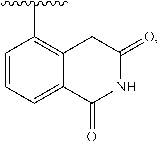 [SMILES: *c1cccc2c1CC(=O)NC2=O] | (dd) |
| 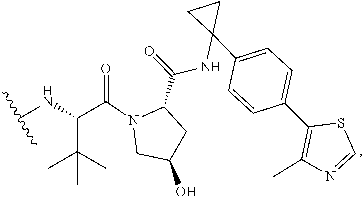 [SMILES: *N[C@H](C(=O)N1C[C@H](O)C[C@H]1C(=O)NC1(c2ccc(-c3scnc3C)cc2)CC1)C(C)(C)C] | (ee) |
| 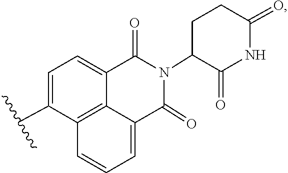 [SMILES: *c1ccc2c3c(cccc13)C(=O)N(C1CCC(=O)NC1=O)C2=O] | (ff) |
| 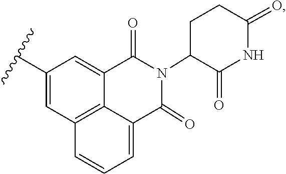 [SMILES: *c1cc2c3c(cccc3c1)C(=O)N(C1CCC(=O)NC1=O)C2=O] | (gg) |
| 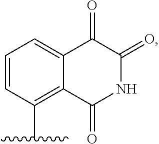 [SMILES: *c1cccc2c1C(=O)NC(=O)C2=O] | (hh) |
| 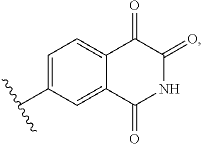 [SMILES: *c1ccc2c(c1)C(=O)NC(=O)C2=O] | (ii) |
| 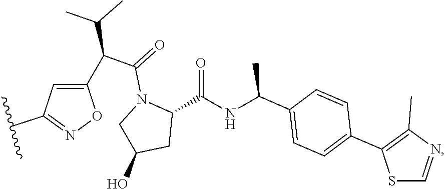 [SMILES: *c1cc([C@H](C(=O)N2C[C@H](O)C[C@H]2C(=O)N[C@@H](C)c2ccc(-c3scnc3C)cc2)C(C)C)on1] | (jj) |
| 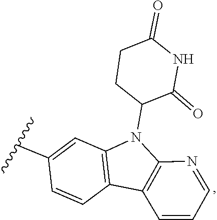 [SMILES: *c1ccc2c3cccnc3n(C3CCC(=O)NC3=O)c2c1] | (kk) |
| 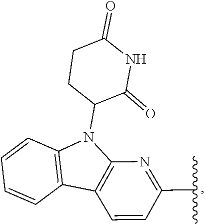 [SMILES: *c1ccc2c3ccccc3n(C3CCC(=O)NC3=O)c2n1] | (ll) |
| 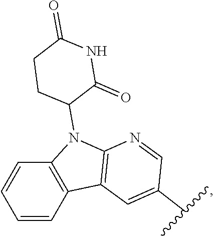 [SMILES: *c1cnc2c(c1)c1ccccc1n2C1CCC(=O)NC1=O] | (mm) |
| 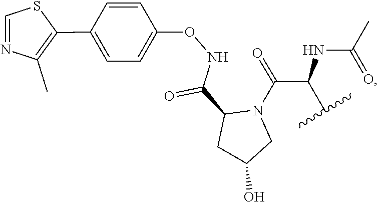 [SMILES: *[C@H](NC(C)=O)C(=O)N1C[C@H](O)C[C@H]1C(=O)NOc1ccc(-c2scnc2C)cc1] | (nn) |
| 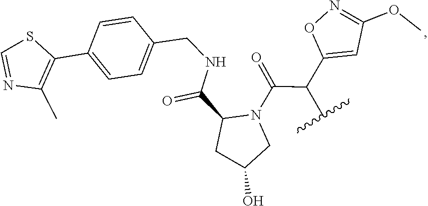 [SMILES: *C(C(=O)N1C[C@H](O)C[C@H]1C(=O)NCc1ccc(-c2scnc2C)cc1)c1cc(OC)no1] | (oo) |
| 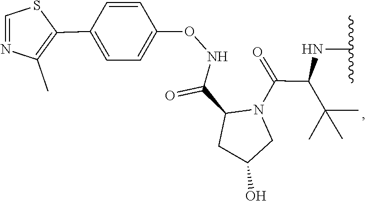 [SMILES: *N[C@H](C(=O)N1C[C@H](O)C[C@H]1C(=O)NOc1ccc(-c2scnc2C)cc1)C(C)(C)C] | (pp) |
| 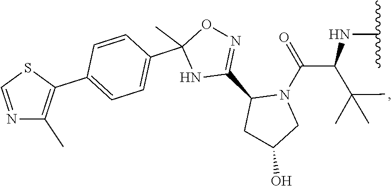 [SMILES: *N[C@H](C(=O)N1C[C@H](O)C[C@H]1C1=NOC(C)(c2ccc(-c3scnc3C)cc2)N1)C(C)(C)C] | (qq) |
| 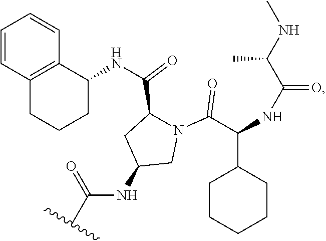 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1] | (rr) |
| 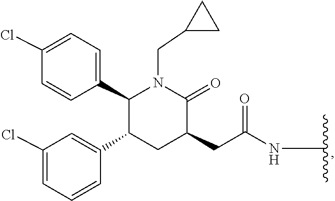 [SMILES: *NC(=O)C[C@H]1C[C@H](c2cccc(Cl)c2)[C@@H](c2ccc(Cl)cc2)N(CC2CC2)C1=O] | (ss) |
| 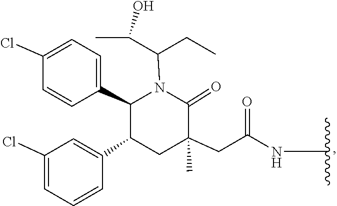 [SMILES: *NC(=O)C[C@@]1(C)C[C@H](c2cccc(Cl)c2)[C@@H](c2ccc(Cl)cc2)N(C(CC)[C@H](C)O)C1=O] | (tt) |
| 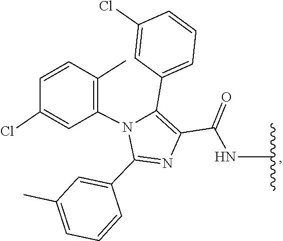 [SMILES: *NC(=O)c1nc(-c2cccc(C)c2)n(-c2cc(Cl)ccc2C)c1-c1cccc(Cl)c1] | (uu) |
| 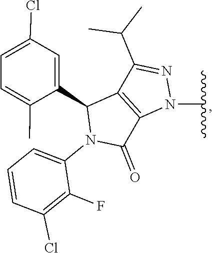 [SMILES: *n1nc(C(C)C)c2c1C(=O)N(c1cccc(Cl)c1F)[C@H]2c1cc(Cl)ccc1C] | (vv) |
| 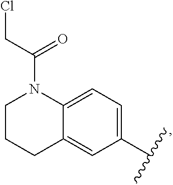 [SMILES: *c1ccc2c(c1)CCCN2C(=O)CCl] | (ww) |
| 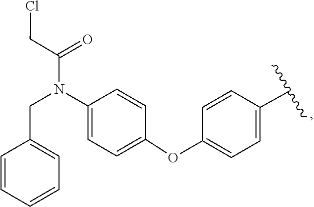 [SMILES: *c1ccc(Oc2ccc(N(Cc3ccccc3)C(=O)CCl)cc2)cc1] | (xx) |
| 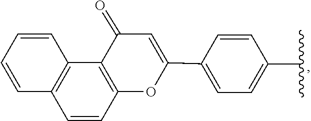 [SMILES: *c1ccc(-c2cc(=O)c3c(ccc4ccccc43)o2)cc1] | (vv) |
| 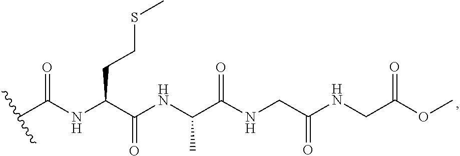 [SMILES: *C(=O)N[C@@H](CCSC)C(=O)N[C@@H](C)C(=O)NCC(=O)NCC(=O)OC] | (zz) |
| 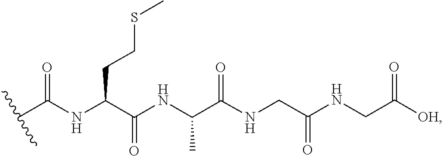 [SMILES: *C(=O)N[C@@H](CCSC)C(=O)N[C@@H](C)C(=O)NCC(=O)NCC(=O)O] | (aaa) |
| 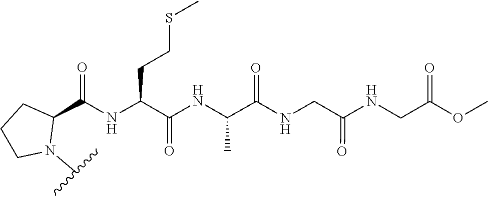 [SMILES: *N1CCC[C@H]1C(=O)N[C@@H](CCSC)C(=O)N[C@@H](C)C(=O)NCC(=O)NCC(=O)OC] | (bbb) |
| 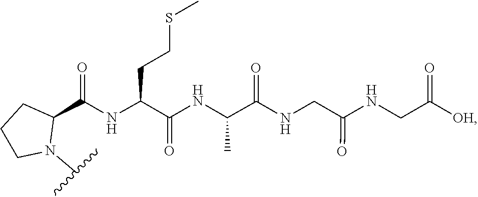 [SMILES: *N1CCC[C@H]1C(=O)N[C@@H](CCSC)C(=O)N[C@@H](C)C(=O)NCC(=O)NCC(=O)O] | (ccc) |
| 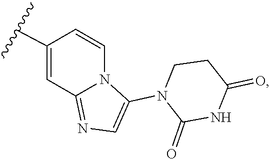 [SMILES: *c1ccn2c(N3CCC(=O)NC3=O)cnc2c1] | (ddd) |
| 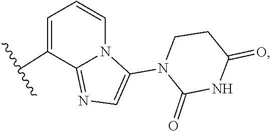 [SMILES: *c1cccn2c(N3CCC(=O)NC3=O)cnc12] | (eee) |
| 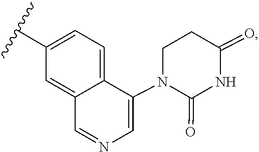 [SMILES: *c1ccc2c(N3CCC(=O)NC3=O)cncc2c1] | (fff) |
| 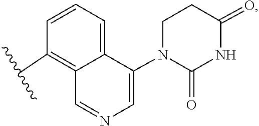 [SMILES: *c1cccc2c(N3CCC(=O)NC3=O)cncc12] | (ggg) |
| 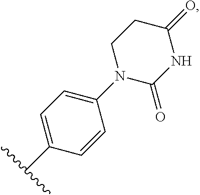 [SMILES: *c1ccc(N2CCC(=O)NC2=O)cc1] | (hhh) |
| 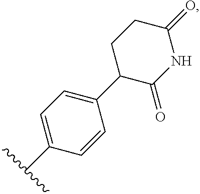 [SMILES: *c1ccc(C2CCC(=O)NC2=O)cc1] | (iii) |
| 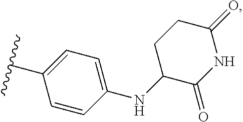 [SMILES: *c1ccc(NC2CCC(=O)NC2=O)cc1] | (jjj) |
| 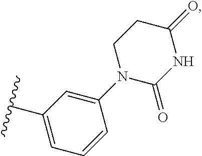 [SMILES: *c1cccc(N2CCC(=O)NC2=O)c1] | (kkk) |
| 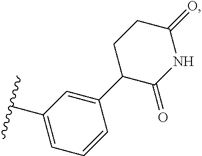 [SMILES: *c1cccc(C2CCC(=O)NC2=O)c1] | (lll) |
| 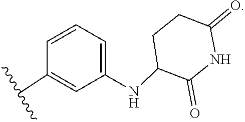 [SMILES: *c1cccc(NC2CCC(=O)NC2=O)c1] | (mmm) |

TABLE B
Exemplified Linkers (L)

| | |
|---|---|
| 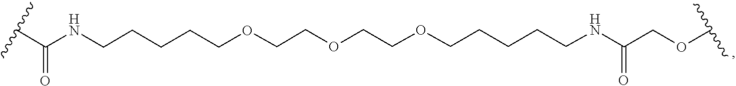 [SMILES: *OCC(=O)NCCCCCOCCOCCOCCCCCNC(*)=O] | (1) |
| 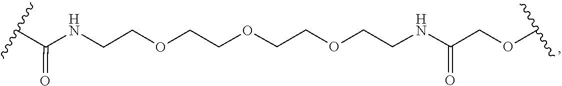 [SMILES: *OCC(=O)NCCOCCOCCOCCNC(*)=O] | (2) |
| 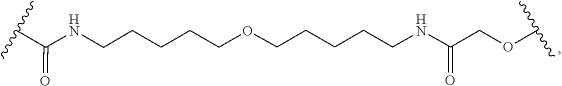 [SMILES: *OCC(=O)NCCCCCOCCCCCNC(*)=O] | (3) |
| 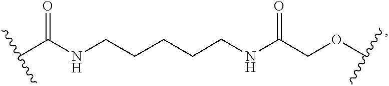 [SMILES: *OCC(=O)NCCCCCNC(*)=O] | (4) |
| 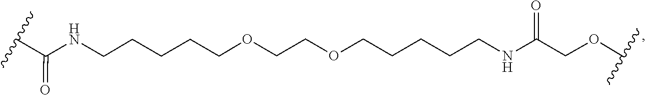 [SMILES: *OCC(=O)NCCCCCOCCOCCCCCNC(*)=O] | (5) |
| 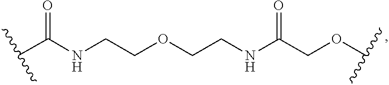 [SMILES: *OCC(=O)NCCOCCNC(*)=O] | (6) |
| 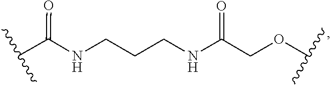 [SMILES: *OCC(=O)NCCCNC(*)=O] | (7) |
| 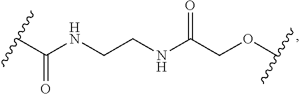 [SMILES: *OCC(=O)NCCNC(*)=O] | (8) |
| 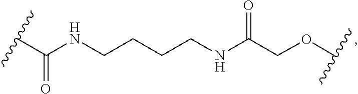 [SMILES: *OCC(=O)NCCCCNC(*)=O] | (9) |
| 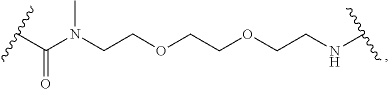 [SMILES: *NCCOCCOCCN(C)C(*)=O] | (10) |
| 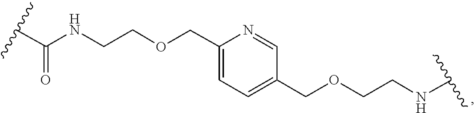 [SMILES: *NCCOCc1ccc(COCCNC(*)=O)nc1] | (11) |
| 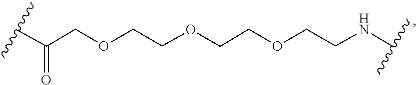 [SMILES: *NCCOCCOCCOCC(*)=O] | (12) |
| 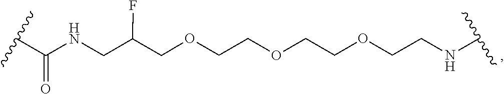 [SMILES: *NCCOCCOCCOCC(F)CNC(*)=O] | (13) |
| 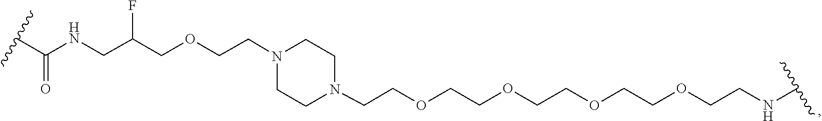 [SMILES: *NCCOCCOCCOCCOCCN1CCN(CCOCC(F)CNC(*)=O)CC1] | (14) |
| 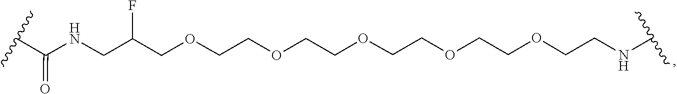 [SMILES: *NCCOCCOCCOCCOCCOCC(F)CNC(*)=O] | (15) |
| 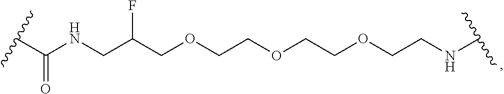 [SMILES: *NCCOCCOCCOCC(F)CNC(*)=O] | (16) |
| 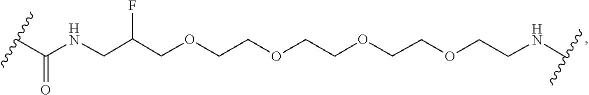 [SMILES: *NCCOCCOCCOCCOCC(F)CNC(*)=O] | (17) |
| 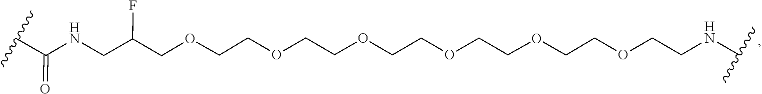 [SMILES: *NCCOCCOCCOCCOCCOCCOCC(F)CNC(*)=O] | (18) |
| 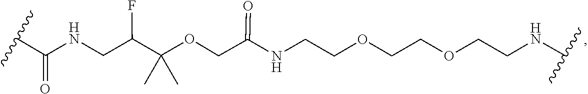 [SMILES: *NCCOCCOCCNC(=O)COC(C)(C)C(F)CNC(*)=O] | (19) |
| 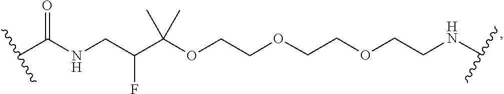 [SMILES: *NCCOCCOCCOC(C)(C)C(F)CNC(*)=O] | (20) |
| 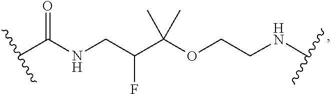 [SMILES: *NCCOC(C)(C)C(F)CNC(*)=O] | (21) |
| 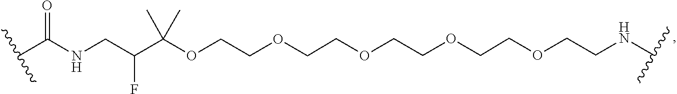 [SMILES: *NCCOCCOCCOCCOCCOC(C)(C)C(F)CNC(*)=O] | (22) |
| 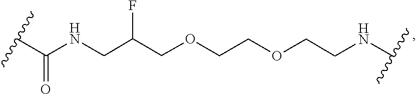 [SMILES: *NCCOCCOCC(F)CNC(*)=O] | (23) |
| 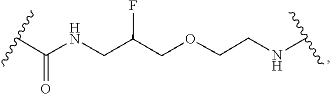 [SMILES: *NCCOCC(F)CNC(*)=O] | (24) |
| 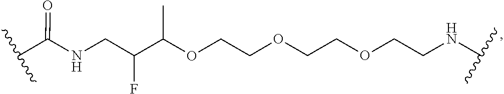 [SMILES: *NCCOCCOCCOC(C)C(F)CNC(*)=O] | (25) |
| 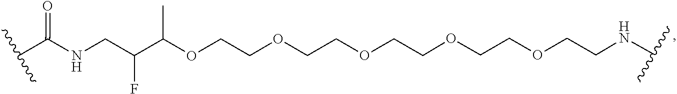 [SMILES: *NCCOCCOCCOCCOCCOC(C)C(F)CNC(*)=O] | (26) |
| 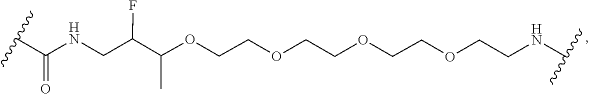 [SMILES: *NCCOCCOCCOCCOC(C)C(F)CNC(*)=O] | (27) |
| 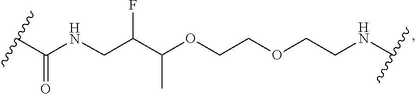 [SMILES: *NCCOCCOC(C)C(F)CNC(*)=O] | (28) |
| 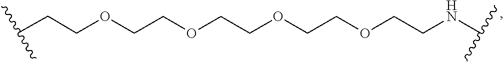 [SMILES: *CCOCCOCCOCCOCCN*] | (29) |
| 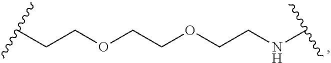 [SMILES: *CCOCCOCCN*] | (30) |
| 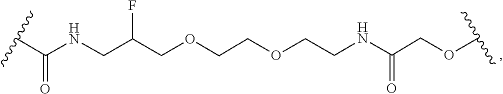 [SMILES: *OCC(=O)NCCOCCOCC(F)CNC(*)=O] | (31) |
| 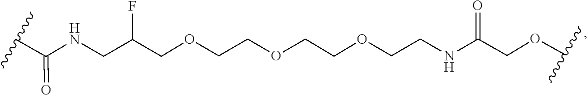 [SMILES: *OCC(=O)NCCOCCOCCOCC(F)CNC(*)=O] | (32) |
| 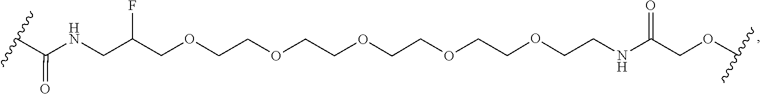 [SMILES: *OCC(=O)NCCOCCOCCOCCOCCOCC(F)CNC(*)=O] | (33) |
| 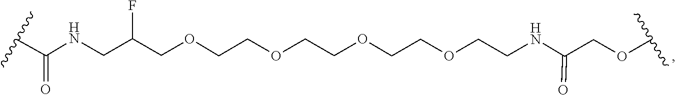 [SMILES: *OCC(=O)NCCOCCOCCOCCOCC(F)CNC(*)=O] | (34) |
| 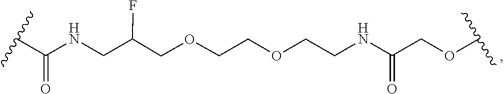 [SMILES: *OCC(=O)NCCOCCOCC(F)CNC(*)=O] | (35) |
| 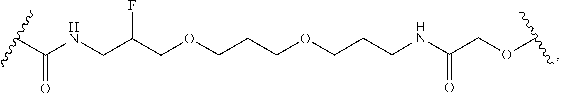 [SMILES: *OCC(=O)NCCCOCCCOCC(F)CNC(*)=O] | (36) |
| 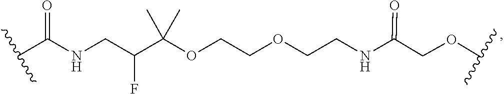 [SMILES: *OCC(=O)NCCOCCOC(C)(C)C(F)CNC(*)=O] | (37) |
| 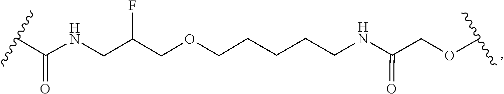 [SMILES: *OCC(=O)NCCCCCOCC(F)CNC(*)=O] | (38) |
| 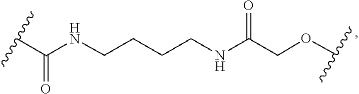 [SMILES: *OCC(=O)NCCCCNC(*)=O] | (39) |
| 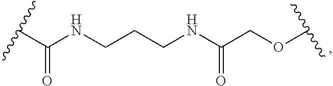 [SMILES: *OCC(=O)NCCCNC(*)=O] | (40) |
| 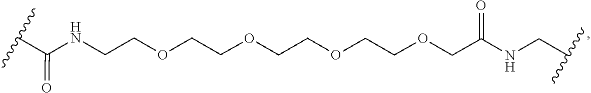 [SMILES: *CNC(=O)COCCOCCOCCOCCNC(*)=O] | (41) |
| 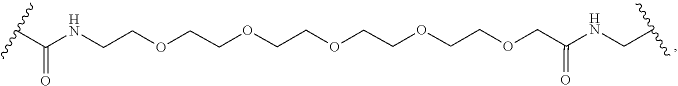 [SMILES: *CNC(=O)COCCOCCOCCOCCOCCNC(*)=O] | (42) |
| 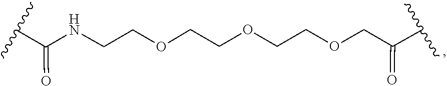 [SMILES: *C(=O)COCCOCCOCCNC(*)=O] | (43) |
| 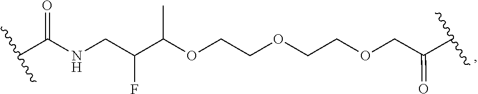 [SMILES: *C(=O)COCCOCCOC(C)C(F)CNC(*)=O] | (44) |
| 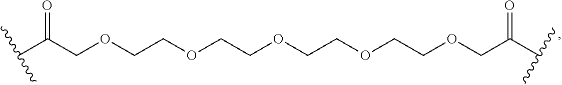 [SMILES: *C(=O)COCCOCCOCCOCCOCC(*)=O] | (45) |
| 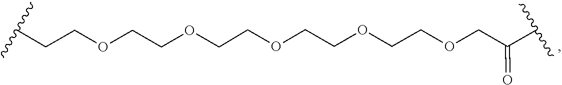 [SMILES: *CCOCCOCCOCCOCCOCC(*)=O] | (46) |
| 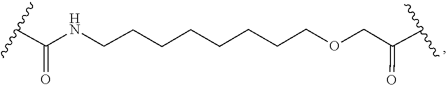 [SMILES: *C(=O)COCCCCCCCCNC(*)=O] | (47) |
| 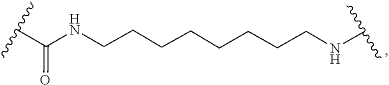 [SMILES: *NCCCCCCCCNC(*)=O] | (49) |
| 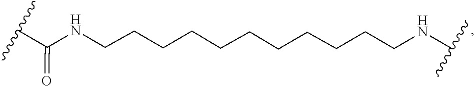 [SMILES: *NCCCCCCCCCCCNC(*)=O] | (50) |
| 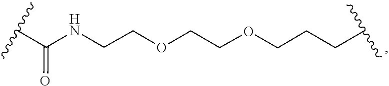 [SMILES: *CCCOCCOCCNC(*)=O] | (51) |
| 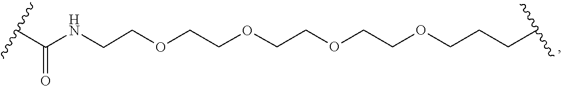 [SMILES: *CCCOCCOCCOCCOCCNC(*)=O] | (52) |
| 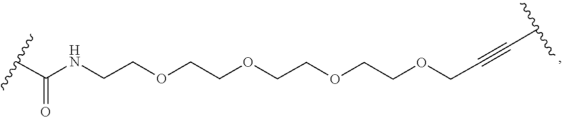 [SMILES: *C#CCOCCOCCOCCOCCNC(*)=O] | (53) |
| 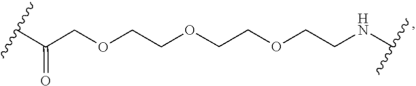 [SMILES: *NCCOCCOCCOCC(*)=O] | (54) |
| 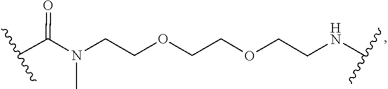 [SMILES: *NCCOCCOCCN(C)C(*)=O] | (55) |
| 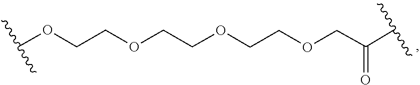 [SMILES: *OCCOCCOCCOCC(*)=O] | (56) |
| 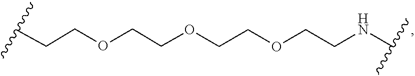 [SMILES: *CCOCCOCCOCCN*] | (57) |
| 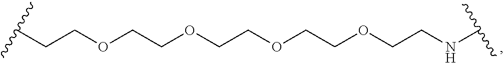 [SMILES: *CCOCCOCCOCCOCCN*] | (58) |
| 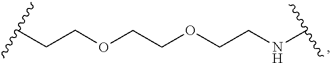 [SMILES: *CCOCCOCCN*] | (59) |
| 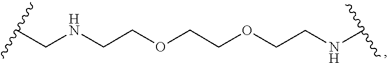 [SMILES: *CNCCOCCOCCN*] | (60) |
| 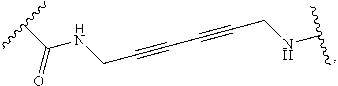 [SMILES: *NCC#CC#CCNC(*)=O] | (61) |
| 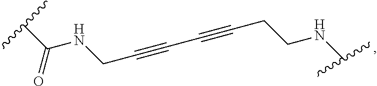 [SMILES: *NCCC#CC#CCNC(*)=O] | (62) |
| 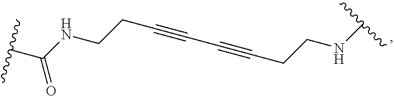 [SMILES: *NCCC#CC#CCCNC(*)=O] | (63) |
| 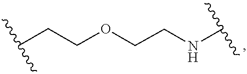 [SMILES: *CCOCCN*] | (64) |
| 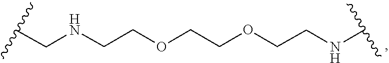 [SMILES: *CNCCOCCOCCN*] | (65) |
| 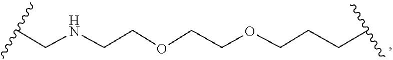 [SMILES: *CCCOCCOCCNC*] | (66) |
| 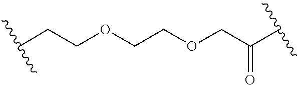 [SMILES: *CCOCCOCC(*)=O] | (67) |
| 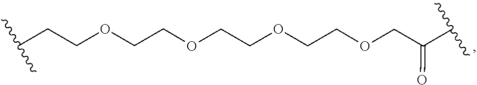 [SMILES: *CCOCCOCCOCCOCC(*)=O] | (68) |
| 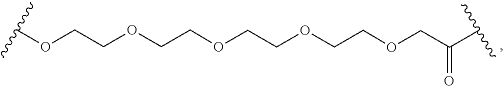 [SMILES: *OCCOCCOCCOCCOCC(*)=O] | (69) |
| 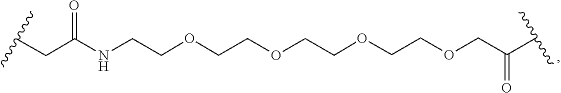 [SMILES: *CC(=O)NCCOCCOCCOCCOCC(*)=O] | (70) |
| 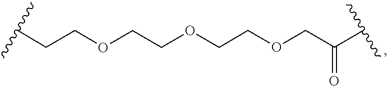 [SMILES: *CCOCCOCCOCC(*)=O] | (71) |
| 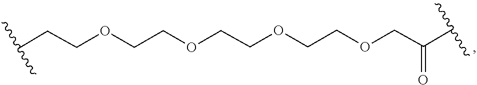 [SMILES: *CCOCCOCCOCCOCC(*)=O] | (72) |
| 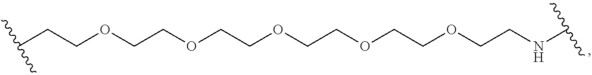 [SMILES: *CCOCCOCCOCCOCCOCCN*] | (73) |
| 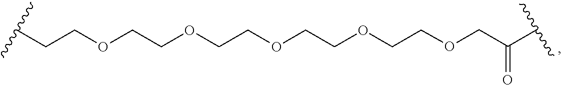 [SMILES: *CCOCCOCCOCCOCCOCC(*)=O] | (74) |
| 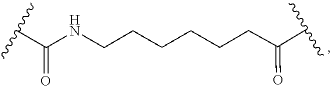 [SMILES: *C(=O)CCCCCCNC(*)=O] | (75) |
| 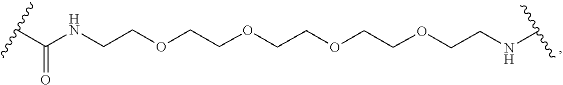 [SMILES: *NCCOCCOCCOCCOCCNC(*)=O] | (76) |
| 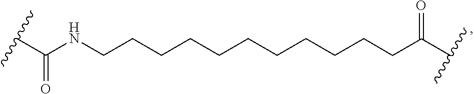 [SMILES: *C(=O)CCCCCCCCCCCNC(*)=O] | (77) |
| 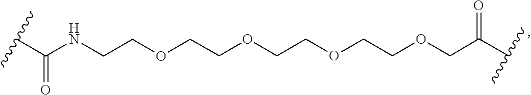 [SMILES: *C(=O)COCCOCCOCCOCCNC(*)=O] | (78) |
| 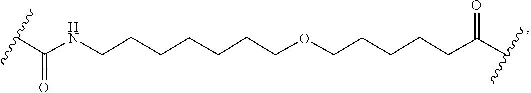 [SMILES: *C(=O)CCCCCOCCCCCCCNC(*)=O] | (79) |
| 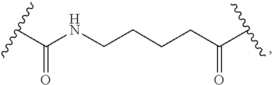 [SMILES: *C(=O)CCCCNC(*)=O] | (80) |
| 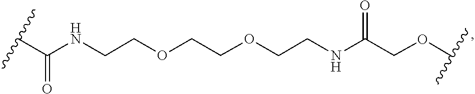 [SMILES: *OCC(=O)NCCOCCOCCNC(*)=O] | (81) |
| 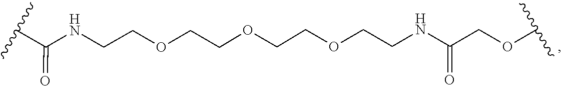 [SMILES: *OCC(=O)NCCOCCOCCOCCNC(*)=O] | (82) |
| 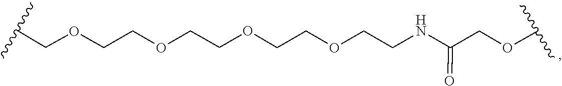 [SMILES: *COCCOCCOCCOCCNC(=O)CO*] | (83) |
| 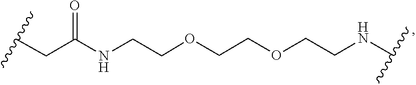 [SMILES: *CC(=O)NCCOCCOCCN*] | (84) |
| 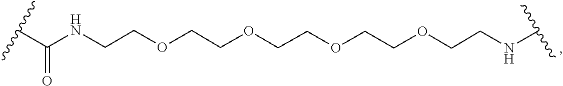 [SMILES: *NCCOCCOCCOCCOCCNC(*)=O] | (85) |
| 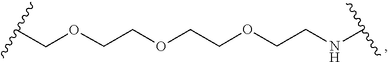 [SMILES: *COCCOCCOCCN*] | (86) |
| 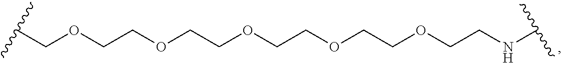 [SMILES: *COCCOCCOCCOCCOCCN*] | (87) |
| 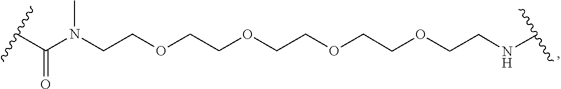 [SMILES: *NCCOCCOCCOCCOCCN(C)C(*)=O] | (88) |
| 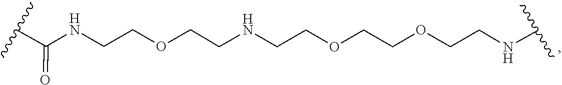 [SMILES: *NCCOCCOCCNCCOCCNC(*)=O] | (89) |
| 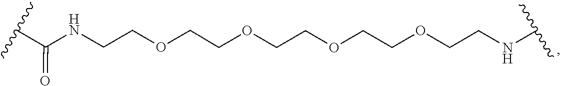 [SMILES: *NCCOCCOCCOCCOCCNC(*)=O] | (90) |
| 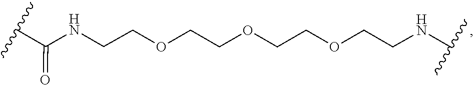 [SMILES: *NCCOCCOCCOCCNC(*)=O] | (91) |
| 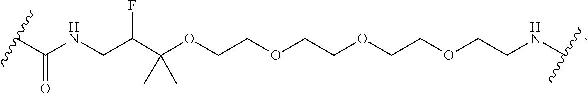 [SMILES: *NCCOCCOCCOCCOC(C)(C)C(F)CNC(*)=O] | (92) |
| 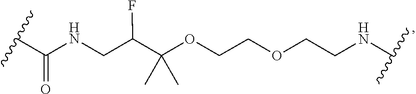 [SMILES: *NCCOCCOC(C)(C)C(F)CNC(*)=O] | (93) |
| 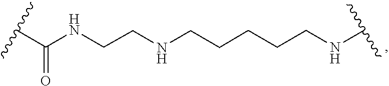 [SMILES: *NCCCCCNCCNC(*)=O] | (94) |
| 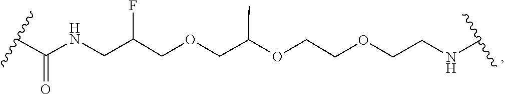 [SMILES: *NCCOCCOC(C)COCC(F)CNC(*)=O] | (95) |
| 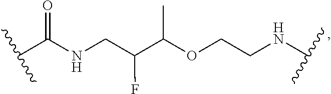 [SMILES: *NCCOC(C)C(F)CNC(*)=O] | (96) |
| 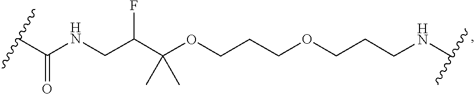 [SMILES: *NCCCOCCCOC(C)(C)C(F)CNC(*)=O] | (97) |
| 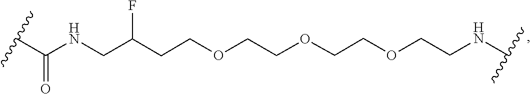 [SMILES: *NCCOCCOCCOCCC(F)CNC(*)=O] | (98) |
| 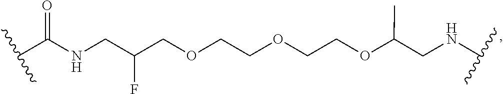 [SMILES: *NCC(C)OCCOCCOCC(F)CNC(*)=O] | (99) |
| 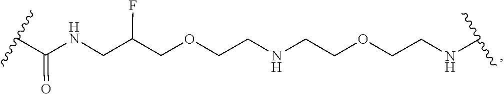 [SMILES: *NCCOCCNCCOCC(F)CNC(*)=O] | (100) |
| 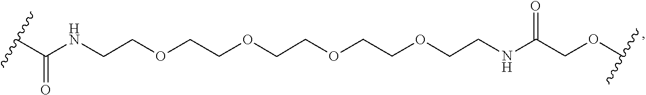 [SMILES: *OCC(=O)NCCOCCOCCOCCOCCNC(*)=O] | (101) |
| 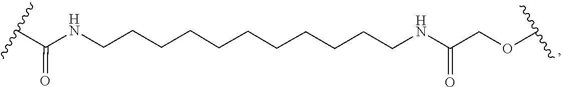 [SMILES: *OCC(=O)NCCCCCCCCCCCNC(*)=O] | (102) |
| 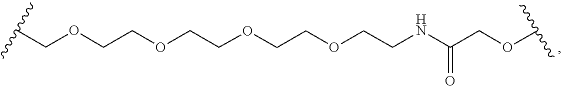 [SMILES: *COCCOCCOCCOCCNC(=O)CO*] | (103) |
| 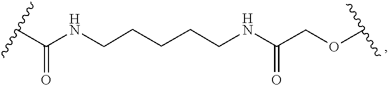 [SMILES: *OCC(=O)NCCCCCNC(*)=O] | (104) |
| 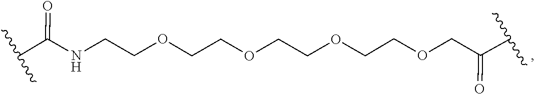 [SMILES: *C(=O)COCCOCCOCCOCCNC(*)=O] | (105) |
| 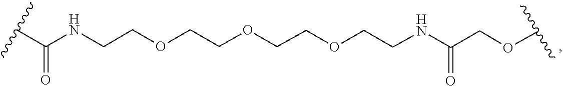 [SMILES: *OCC(=O)NCCOCCOCCOCCNC(*)=O] | (106) |
| 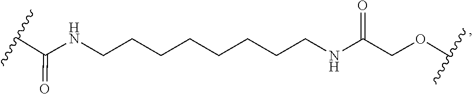 [SMILES: *OCC(=O)NCCCCCCCCNC(*)=O] | (107) |
| 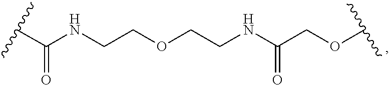 [SMILES: *OCC(=O)NCCOCCNC(*)=O] | (108) |
| 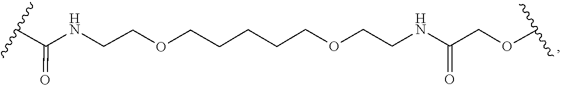 [SMILES: *OCC(=O)NCCOCCCCCOCCNC(*)=O] | (109) |
| 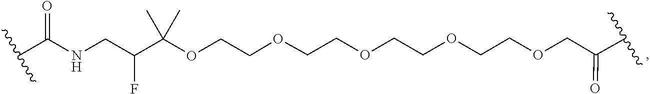 [SMILES: *C(=O)COCCOCCOCCOCCOC(C)(C)C(F)CNC(*)=O] | (110) |
| 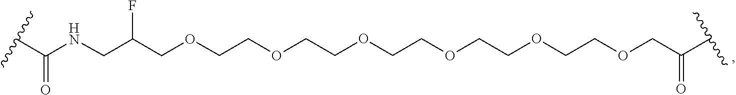 [SMILES: *C(=O)COCCOCCOCCOCCOCCOCC(F)CNC(*)=O] | (111) |
| 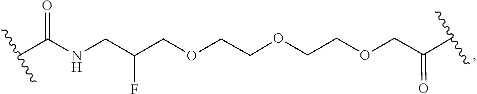 [SMILES: *C(=O)COCCOCCOCC(F)CNC(*)=O] | (112) |
| 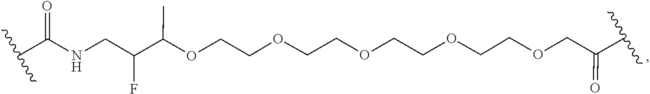 [SMILES: *C(=O)COCCOCCOCCOCCOC(C)C(F)CNC(*)=O] | (113) |
| 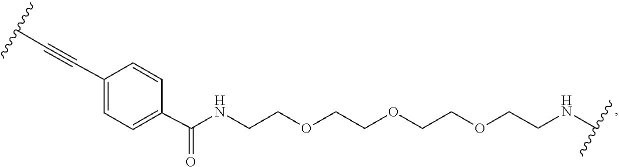 [SMILES: *C#Cc1ccc(C(=O)NCCOCCOCCOCCN*)cc1] | (114) |
| 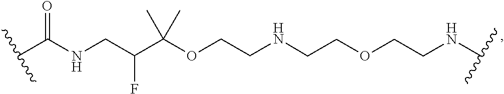 [SMILES: *NCCOCCNCCOC(C)(C)C(F)CNC(*)=O] | (115) |
| 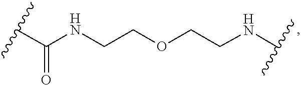 [SMILES: *NCCOCCNC(*)=O] | (116) |
| 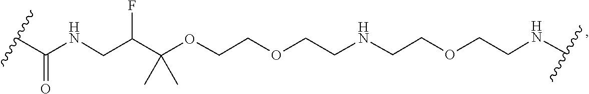 [SMILES: *NCCOCCNCCOCCOC(C)(C)C(F)CNC(*)=O] | (117) |
| 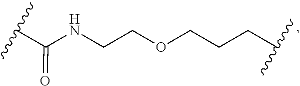 [SMILES: *CCCOCCNC(*)=O] | (118) |
| 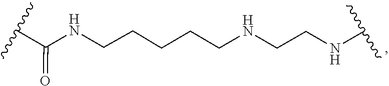 [SMILES: *NCCNCCCCCNC(*)=O] | |
| 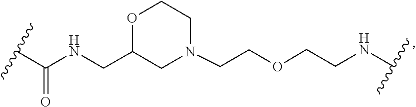 [SMILES: *NCCOCCN1CCOC(CNC(*)=O)C1] | (119) |
| 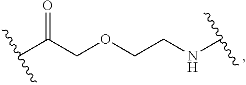 [SMILES: *NCCOCC(*)=O] | |
| 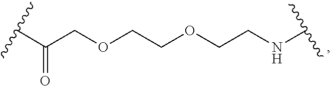 [SMILES: *NCCOCCOCC(*)=O] | (120) |
| 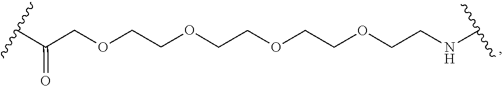 [SMILES: *NCCOCCOCCOCCOCC(*)=O] | (121) |
| 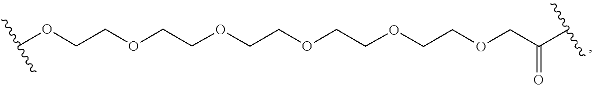 [SMILES: *OCCOCCOCCOCCOCCOCC(*)=O] | (122) |
| 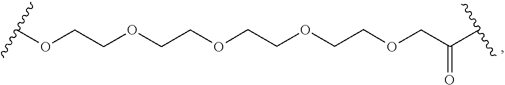 [SMILES: *OCCOCCOCCOCCOCC(*)=O] | (123) |
| 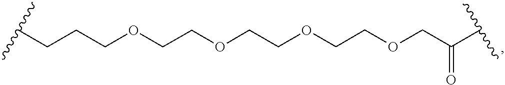 [SMILES: *CCCOCCOCCOCCOCC(*)=O] | (124) |
| 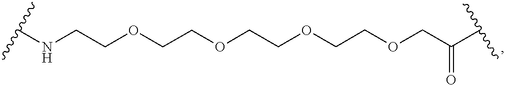 [SMILES: *NCCOCCOCCOCCOCC(*)=O] | (125) |
| 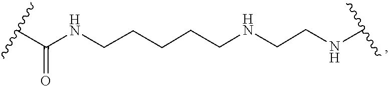 [SMILES: *NCCNCCCCCNC(*)=O] | (126) |
| 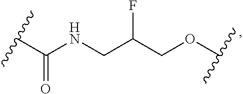 [SMILES: *OCC(F)CNC(*)=O] | |
| 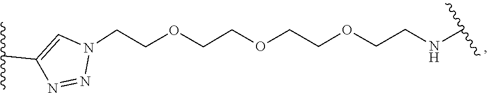 [SMILES: *NCCOCCOCCOCCn1cc(*)nn1] | (127) |
| 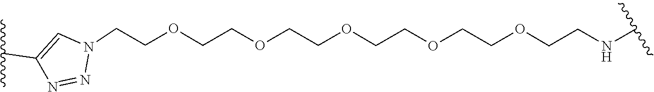 [SMILES: *NCCOCCOCCOCCOCCOCCn1cc(*)nn1] | (128) |
| 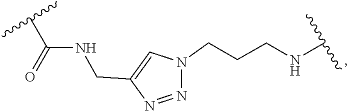 [SMILES: *NCCCn1cc(CNC(*)=O)nn1] | (129) |
| 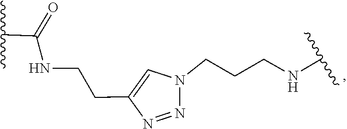 [SMILES: *NCCCn1cc(CCNC(*)=O)nn1] | (130) |
| 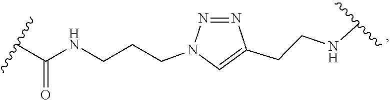 [SMILES: *NCCc1cn(CCCNC(*)=O)nn1] | (131) |
| 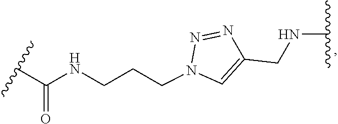 [SMILES: *NCc1cn(CCCNC(*)=O)nn1] | (132) |
| 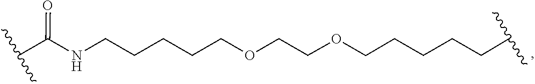 [SMILES: *CCCCCOCCOCCCCCNC(*)=O] | (133) |
| 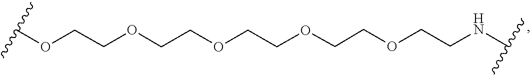 [SMILES: *NCCOCCOCCOCCOCCO*] | (134) |
| 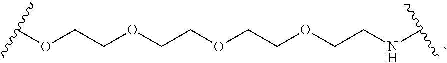 [SMILES: *NCCOCCOCCOCCO*] | (135) |
| 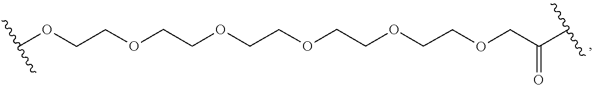 [SMILES: *OCCOCCOCCOCCOCCOCC(*)=O] | (136) |
| 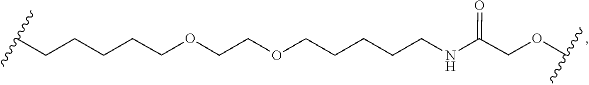 [SMILES: *CCCCCOCCOCCCCCNC(=O)CO*] | (137) |
| 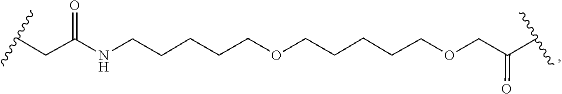 [SMILES: *CC(=O)NCCCCCOCCCCCOCC(*)=O] | (138) |
| 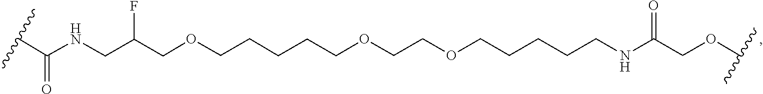 [SMILES: *OCC(=O)NCCCCCOCCOCCCCCOCC(F)CNC(*)=O] | (139) |
| 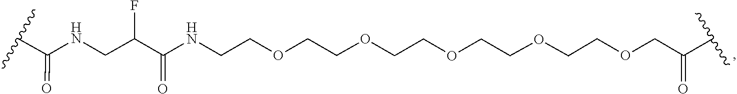 [SMILES: *C(=O)COCCOCCOCCOCCOCCNC(=O)C(F)CNC(*)=O] | (140) |
| 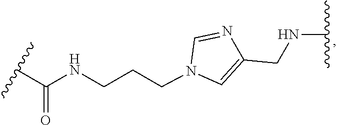 [SMILES: *NCc1cn(CCCNC(*)=O)cn1] | (141) |
| 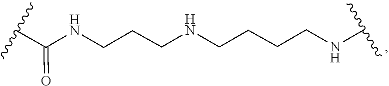 [SMILES: *NCCCCNCCCNC(*)=O] | (142) |
| 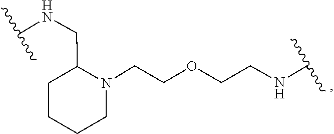 [SMILES: *NCCOCCN1CCCCC1CN*] | (143) |
| 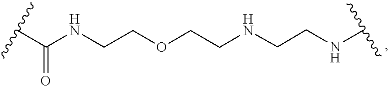 [SMILES: *NCCNCCOCCNC(*)=O] | (144) |
| 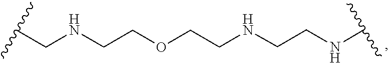 [SMILES: *CNCCOCCNCCN*] | (145) |
| 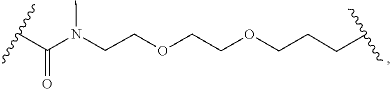 [SMILES: *CCCOCCOCCN(C)C(*)=O] | (146) |
| 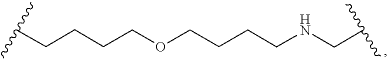 [SMILES: *CCCCOCCCCNC*] | (147) |
| 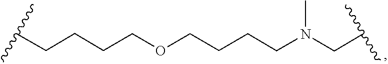 [SMILES: *CCCCOCCCCN(C)C*] | (148) |
| 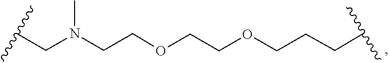 [SMILES: *CCCOCCOCCN(C)C*] | (149) |
| 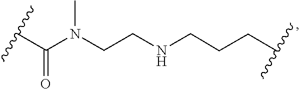 [SMILES: *CCCNCCN(C)C(*)=O] | (150) |
| 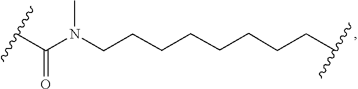 [SMILES: *CCCCCCCCN(C)C(*)=O] | (151) |
| 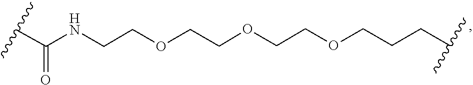 [SMILES: *CCCOCCOCCOCCNC(*)=O] | (152) |
| 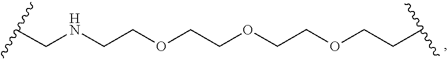 [SMILES: *CCOCCOCCOCCNC*] | (153) |
| 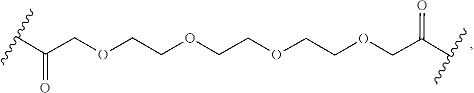 [SMILES: *C(=O)COCCOCCOCCOCC(*)=O] | (154) |
| 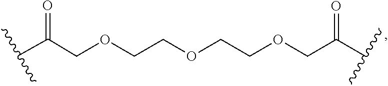 [SMILES: *C(=O)COCCOCCOCC(*)=O] | (155) |
| 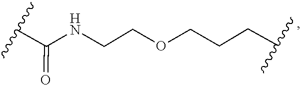 [SMILES: *CCCOCCNC(*)=O] | (156) |
| 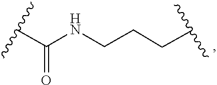 [SMILES: *CCCNC(*)=O] | (157) |
| 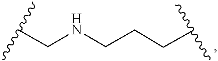 [SMILES: *CCCNC*] | (158) |
| 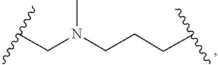 [SMILES: *CCCN(C)C*] | (159) |
| 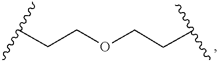 [SMILES: *CCOCC*] | (160) |
| 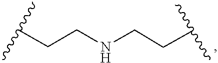 [SMILES: *CCNCC*] | (161) |
| 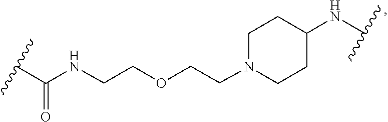 [SMILES: *NC1CCN(CCOCCNC(*)=O)CC1] | (162) |
| 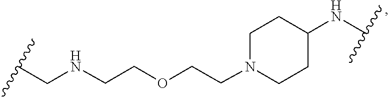 [SMILES: *CNCCOCCN1CCC(N*)CC1] | (163) |
| 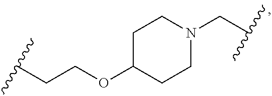 [SMILES: *CCOC1CCN(C*)CC1] | (164) |
| 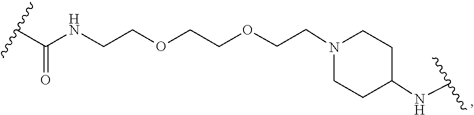 [SMILES: *NC1CCN(CCOCCOCCNC(*)=O)CC1] | (165) |
| 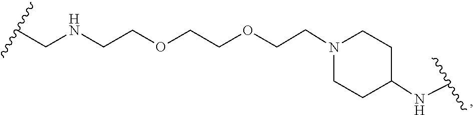 [SMILES: *CNCCOCCOCCN1CCC(N*)CC1] | (166) |
| 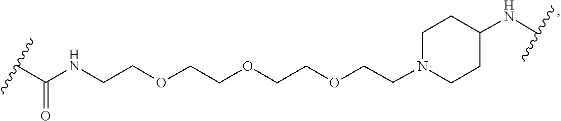 [SMILES: *NC1CCN(CCOCCOCCOCCNC(*)=O)CC1] | (167) |
| 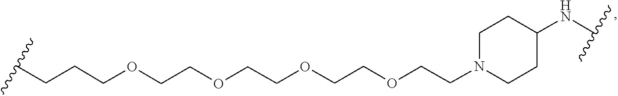 [SMILES: *CCCOCCOCCOCCOCCN1CCC(N*)CC1] | (168) |
| 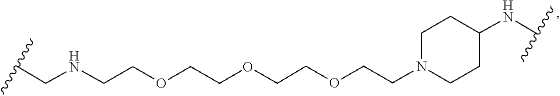 [SMILES: *CNCCOCCOCCOCCN1CCC(N*)CC1] | (169) |
| 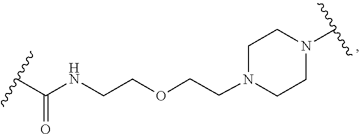 [SMILES: *C(=O)NCCOCCN1CCN(*)CC1] | (170) |
| 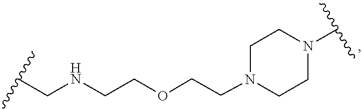 [SMILES: *CNCCOCCN1CCN(*)CC1] | (171) |
| 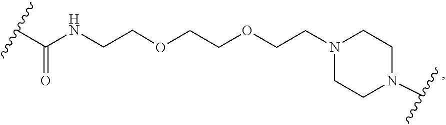 [SMILES: *C(=O)NCCOCCOCCN1CCN(*)CC1] | (172) |
| 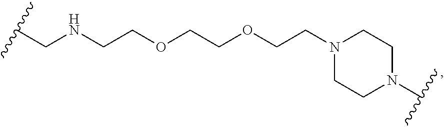 [SMILES: *CNCCOCCOCCN1CCN(*)CC1] | (173) |
| 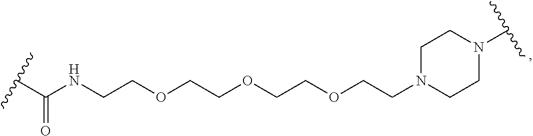 [SMILES: *C(=O)NCCOCCOCCOCCN1CCN(*)CC1] | (174) |
| 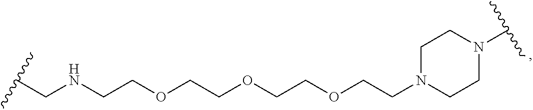 [SMILES: *CNCCOCCOCCOCCN1CCN(*)CC1] | (175) |
| 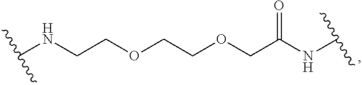 [SMILES: *NCCOCCOCC(=O)N*] | (176) |
| 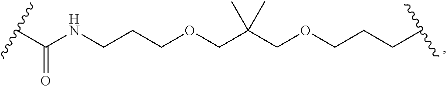 [SMILES: *CCCOCC(C)(C)COCCCNC(*)=O] | (177) |
| 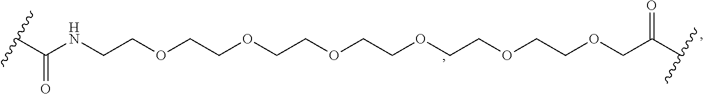 [SMILES: *C(=O)COCCOCCOCCOCCOCCOCCNC(*)=O] | (178) |
| 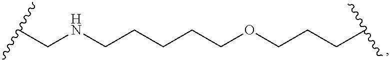 [SMILES: *CCCOCCCCCNC*] | (179) |
| 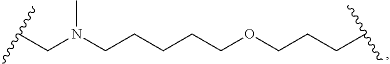 [SMILES: *CCCOCCCCCN(C)C*] | (180) |
| 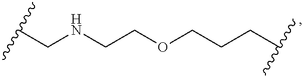 [SMILES: *CCCOCCNC*] | (181) |
| 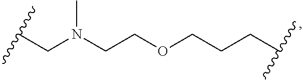 [SMILES: *CCCOCCN(C)C*] | (182) |
| 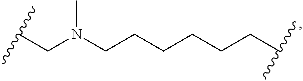 [SMILES: *CCCCCCN(C)C*] | (183) |
| 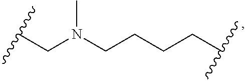 [SMILES: *CCCCN(C)C*] | (184) |
| 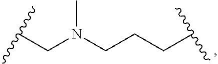 [SMILES: *CCCN(C)C*] | (185) |
| 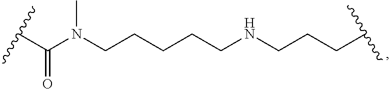 [SMILES: *CCCNCCCCCN(C)C(*)=O] | (186) |
| 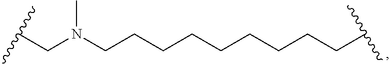 [SMILES: *CCCCCCCCCN(C)C*] | (187) |
| 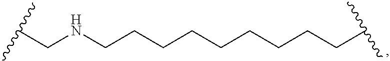 [SMILES: *CCCCCCCCCNC*] | (188) |
| 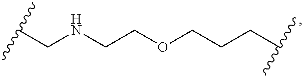 [SMILES: *CCCOCCNC*] | (189) |
| 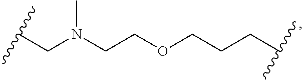 [SMILES: *CCCOCCN(C)C*] | (190) |
| 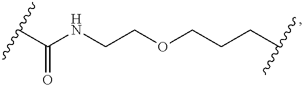 [SMILES: *CCCOCCNC(*)=O] | (191) |
| 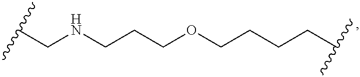 [SMILES: *CCCCOCCCNC*] | (192) |
| 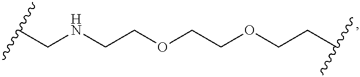 [SMILES: *CCOCCOCCNC*] | (193) |
| 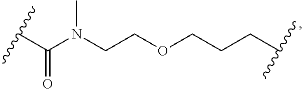 [SMILES: *CCCOCCN(C)C(*)=O] | (194) |
| 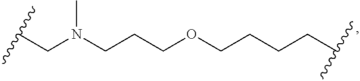 [SMILES: *CCCCOCCCN(C)C*] | (195) |
| 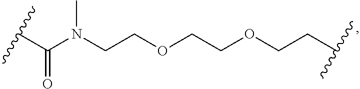 [SMILES: *CCOCCOCCN(C)C(*)=O] | (196) |
| 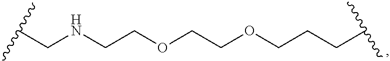 [SMILES: *CCCOCCOCCNC*] | (197) |
| 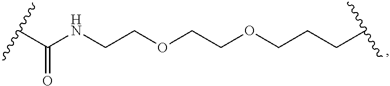 [SMILES: *CCCOCCOCCNC(*)=O] | (198) |
| 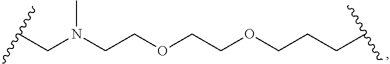 [SMILES: *CCCOCCOCCN(C)C*] | (199) |
| 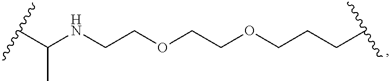 [SMILES: *CCCOCCOCCNC(*)C] | (200) |
| 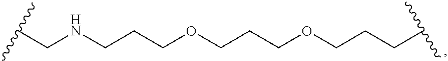 [SMILES: *CCCOCCCOCCCNC*] | (201) |
| 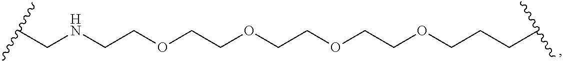 [SMILES: *CCCOCCOCCOCCOCCNC*] | (202) |
| 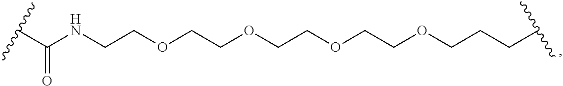 [SMILES: *CCCOCCOCCOCCOCCNC(*)=O] | (203) |
| 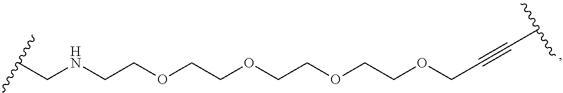 [SMILES: *C#CCOCCOCCOCCOCCNC*] | (204) |
| 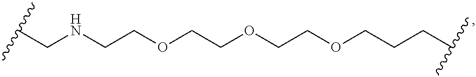 [SMILES: *CCCOCCOCCOCCNC*] | (205) |
| 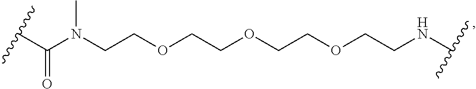 [SMILES: *NCCOCCOCCOCCN(C)C(*)=O] | (206) |
| 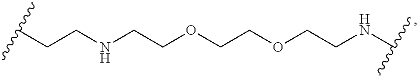 [SMILES: *CCNCCOCCOCCN*] | (207) |
| 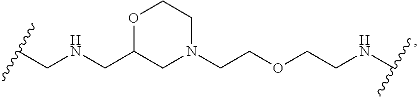 [SMILES: *CNCC1CN(CCOCCN*)CCO1] | (208) |
| 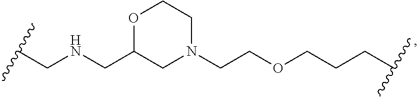 [SMILES: *CCCOCCN1CCOC(CNC*)C1] | (209) |
| 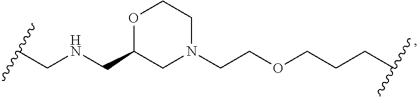 [SMILES: *CCCOCCN1CCO[C@H](CNC*)C1] | (210) |
| 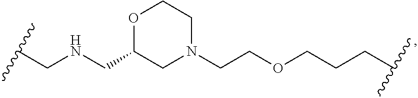 [SMILES: *CCCOCCN1CCO[C@@H](CNC*)C1] | (211) |
| 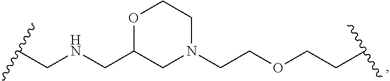 [SMILES: *CCOCCN1CCOC(CNC*)C1] | (212) |
| 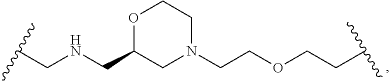 [SMILES: *CCOCCN1CCO[C@H](CNC*)C1] | (213) |
| 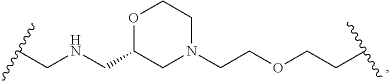 [SMILES: *CCOCCN1CCO[C@@H](CNC*)C1] | (214) |
| 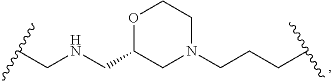 [SMILES: *CCCN1CCO[C@@H](CNC*)C1] | (215) |
| 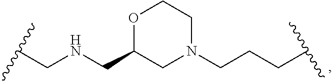 [SMILES: *CCCN1CCO[C@H](CNC*)C1] | (216) |
| 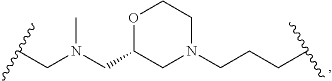 [SMILES: *CCCN1CCO[C@@H](CN(C)C*)C1] | (217) |
| 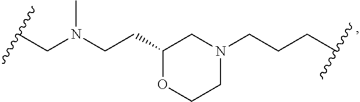 [SMILES: *CCCN1CCO[C@H](CCN(C)C*)C1] | (218) |
| 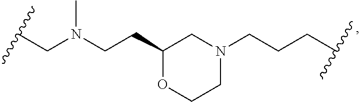 [SMILES: *CCCN1CCO[C@@H](CCN(C)C*)C1] | (219) |
| 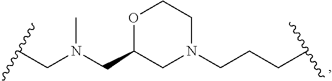 [SMILES: *CCCN1CCO[C@H](CN(C)C*)C1] | (220) |
| 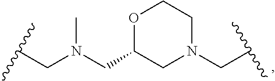 [SMILES: *CN(C)C[C@H]1CN(C*)CCO1] | (221) |
| 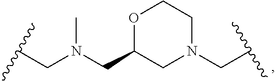 [SMILES: *CN(C)C[C@@H]1CN(C*)CCO1] | (222) |
| 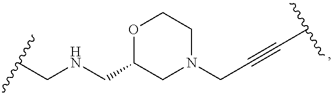 [SMILES: *C#CCN1CCO[C@@H](CNC*)C1] | (223) |
| 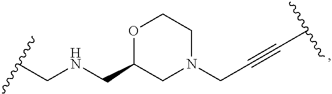 [SMILES: *C#CCN1CCO[C@H](CNC*)C1] | (224) |
| 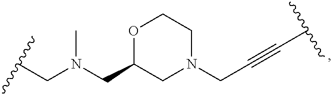 [SMILES: *C#CCN1CCO[C@H](CN(C)C*)C1] | (225) |
| 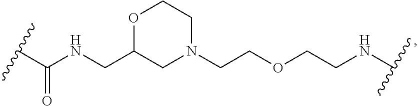 [SMILES: *NCCOCCN1CCOC(CNC(*)=O)C1] | (226) |
| 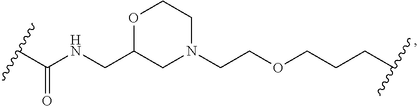 [SMILES: *CCCOCCN1CCOC(CNC(*)=O)C1] | (227) |
| 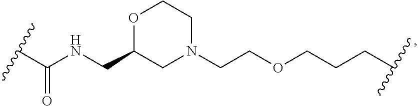 [SMILES: *CCCOCCN1CCO[C@H](CNC(*)=O)C1] | (228) |
| 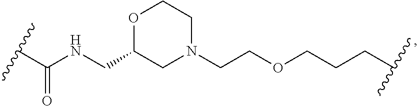 [SMILES: *CCCOCCN1CCO[C@@H](CNC(*)=O)C1] | (229) |
| 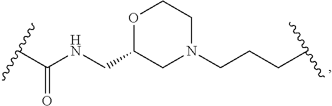 [SMILES: *CCCN1CCO[C@@H](CNC(*)=O)C1] | (230) |
| 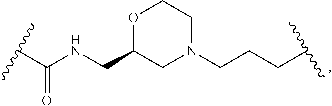 [SMILES: *CCCN1CCO[C@H](CNC(*)=O)C1] | (231) |
| 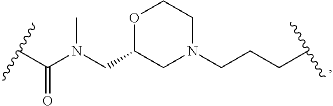 [SMILES: *CCCN1CCO[C@@H](CN(C)C(*)=O)C1] | (232) |
| 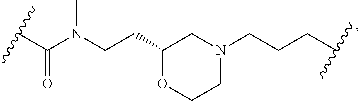 [SMILES: *CCCN1CCO[C@H](CCN(C)C(*)=O)C1] | (233) |
| 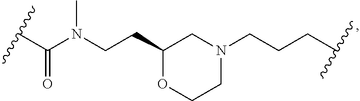 [SMILES: *CCCN1CCO[C@@H](CCN(C)C(*)=O)C1] | (234) |
| 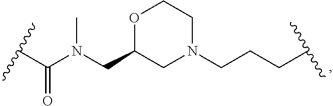 [SMILES: *CCCN1CCO[C@H](CN(C)C(*)=O)C1] | (235) |
| 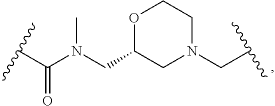 [SMILES: *CN1CCO[C@@H](CN(C)C(*)=O)C1] | (236) |
| 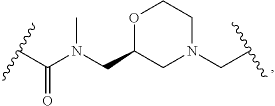 [SMILES: *CN1CCO[C@H](CN(C)C(*)=O)C1] | (237) |
| 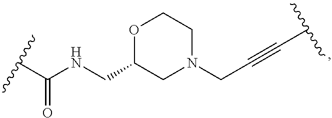 [SMILES: *C#CCN1CCO[C@@H](CNC(*)=O)C1] | (238) |
| 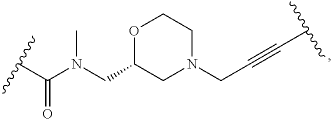 [SMILES: *C#CCN1CCO[C@@H](CN(C)C(*)=O)C1] | (239) |
| 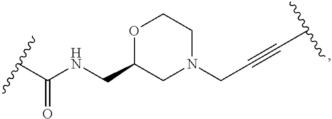 [SMILES: *C#CCN1CCO[C@H](CNC(*)=O)C1] | (240) |
| 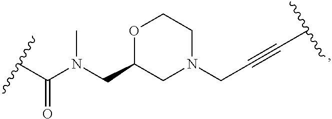 [SMILES: *C#CCN1CCO[C@H](CN(C)C(*)=O)C1] | (241) |
| 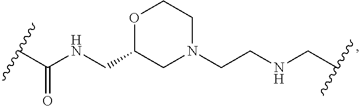 [SMILES: *CNCCN1CCO[C@@H](CNC(*)=O)C1] | (242) |
| 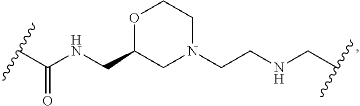 [SMILES: *CNCCN1CCO[C@H](CNC(*)=O)C1] | (243) |
| 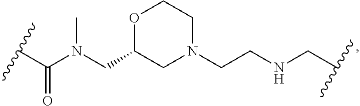 [SMILES: *CNCCN1CCO[C@@H](CN(C)C(*)=O)C1] | (244) |
| 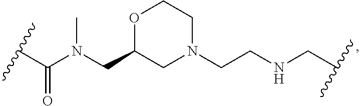 [SMILES: *CNCCN1CCO[C@H](CN(C)C(*)=O)C1] | (245) |
| 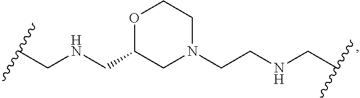 [SMILES: *CNCCN1CCO[C@@H](CNC*)C1] | (246) |
| 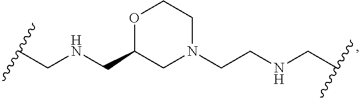 [SMILES: *CNCCN1CCO[C@H](CNC*)C1] | (247) |
| 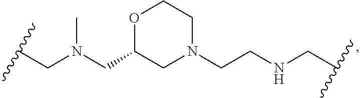 [SMILES: *CNCCN1CCO[C@@H](CN(C)C*)C1] | (248) |
| 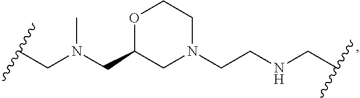 [SMILES: *CNCCN1CCO[C@H](CN(C)C*)C1] | (249) |
| 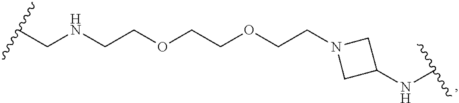 [SMILES: *CNCCOCCOCCN1CC(N*)C1] | (250) |
| 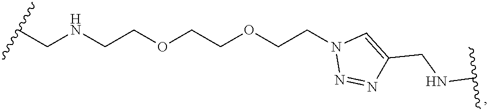 [SMILES: *CNCCOCCOCCn1cc(CN*)nn1] | (251) |
| 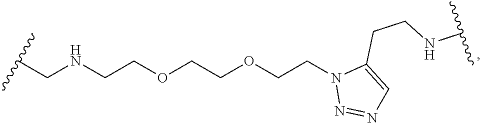 [SMILES: *CNCCOCCOCCn1nncc1CCN*] | (253) |
| 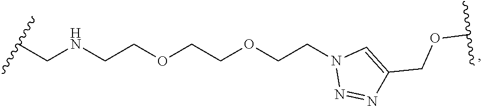 [SMILES: *CNCCOCCOCCn1cc(CO*)nn1] | (254) |
| 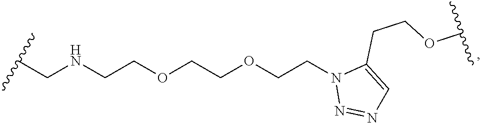 [SMILES: *CNCCOCCOCCn1nncc1CCO*] | (255) |
| 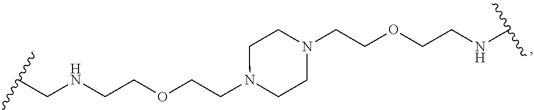 [SMILES: *CNCCOCCN1CCN(CCOCCN*)CC1] | (256) |
| 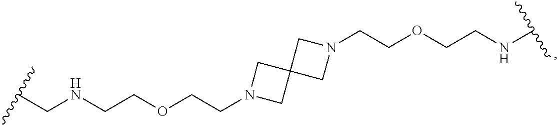 [SMILES: *CNCCOCCN1CC2(CN(CCOCCN*)C2)C1] | (257) |
| 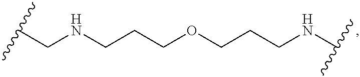 [SMILES: *CNCCCOCCCN*] | (258) |
| 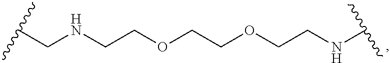 [SMILES: *CNCCOCCOCCN*] | (259) |
| 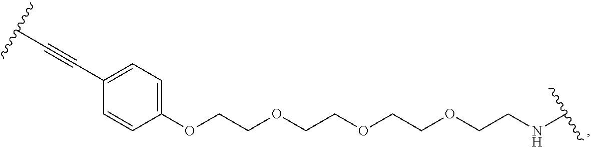 [SMILES: *C#Cc1ccc(OCCOCCOCCOCCN*)cc1] | (260) |
| 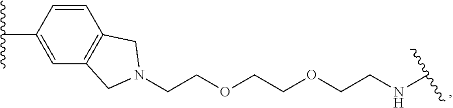 [SMILES: *NCCOCCOCCN1Cc2ccc(*)cc2C1] | (261) |
| 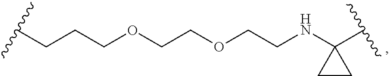 [SMILES: *CCCOCCOCCNC1(*)CC1] | (262) |
| 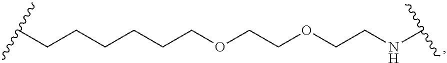 [SMILES: *CCCCCCOCCOCCN*] | (263) |
| 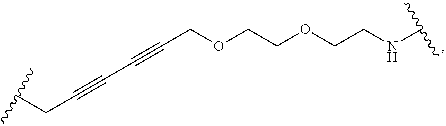 [SMILES: *CC#CC#CCOCCOCCN*] | (264) |
| 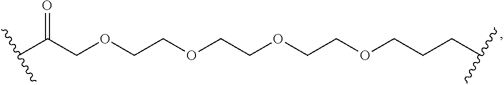 [SMILES: *CCCOCCOCCOCCOCC(*)=O] | (265) |
| 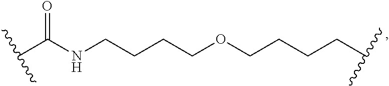 [SMILES: *CCCCOCCCCNC(*)=O] | (266) |
| 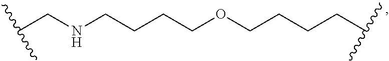 [SMILES: *CCCCOCCCCNC*] | (267) |
| 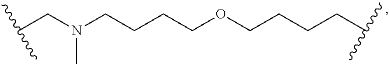 [SMILES: *CCCCOCCCCN(C)C*] | (268) |
| 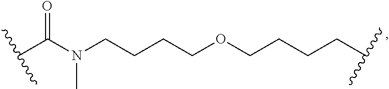 [SMILES: *CCCCOCCCCN(C)C(*)=O] | (269) |
| 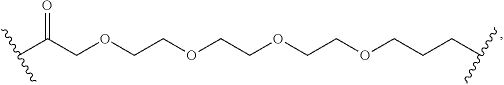 [SMILES: *CCCOCCOCCOCCOCC(*)=O] | (270) |
| 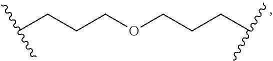 [SMILES: *CCCOCCC*] | (271) |
| 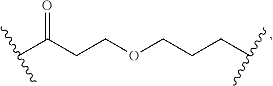 [SMILES: *CCCOCCC(*)=O] | (272) |
| 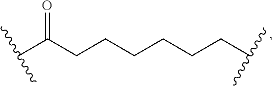 [SMILES: *CCCCCCC(*)=O] | (273) |
| 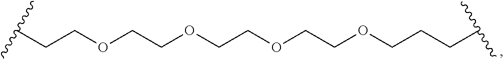 [SMILES: *CCCOCCOCCOCCOCC*] | (274) |
| 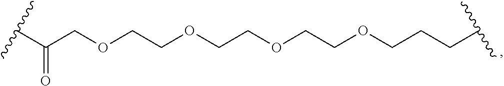 [SMILES: *CCCOCCOCCOCCOCC(*)=O] | (275) |
| 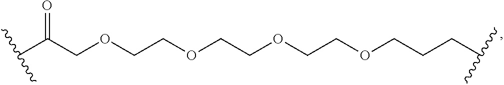 [SMILES: *CCCOCCOCCOCCOCC(*)=O] | (276) |
| 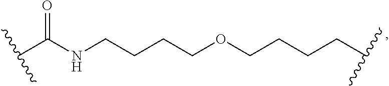 [SMILES: *CCCCOCCCCNC(*)=O] | (277) |
| 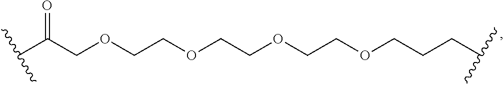 [SMILES: *CCCOCCOCCOCCOCC(*)=O] | (278) |
| 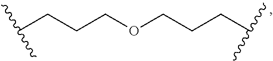 [SMILES: *CCCOCCC*] | (279) |
| 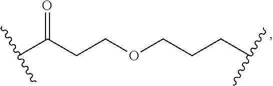 [SMILES: *CCCOCCC(*)=O] | (280) |
| 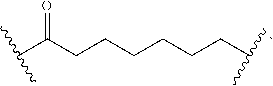 [SMILES: *CCCCCCC(*)=O] | (281) |
| 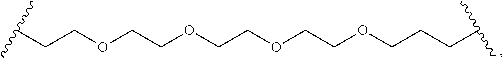 [SMILES: *CCCOCCOCCOCCOCC*] | (282) |
| 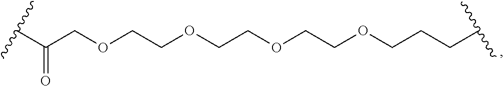 [SMILES: *CCCOCCOCCOCCOCC(*)=O] | (283) |
| 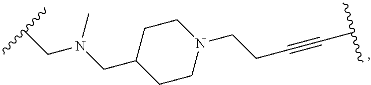 [SMILES: *C#CCCN1CCC(CN(C)C*)CC1] | (284) |
| 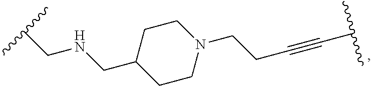 [SMILES: *C#CCCN1CCC(CNC*)CC1] | (285) |
| 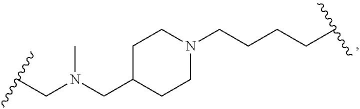 [SMILES: *CCCCN1CCC(CN(C)C*)CC1] | (286) |
| 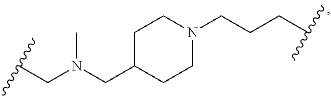 [SMILES: *CCCN1CCC(CN(C)C*)CC1] | (287) |
| 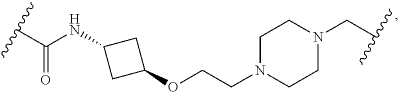 [SMILES: *CN1CCN(CCO[C@H]2C[C@H](NC(*)=O)C2)CC1] | (288) |
| 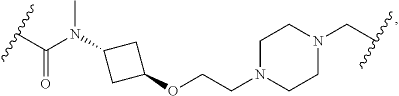 [SMILES: *CN1CCN(CCO[C@H]2C[C@H](N(C)C(*)=O)C2)CC1] | (289) |
| 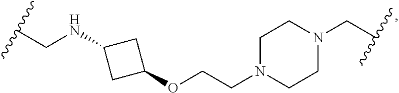 [SMILES: *CN[C@H]1C[C@H](OCCN2CCN(C*)CC2)C1] | (290) |
| 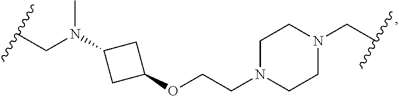 [SMILES: *CN1CCN(CCO[C@H]2C[C@H](N(C)C*)C2)CC1] | (291) |
| 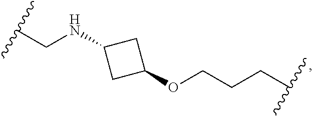 [SMILES: *CCCO[C@H]1C[C@H](NC*)C1] | (292) |
| 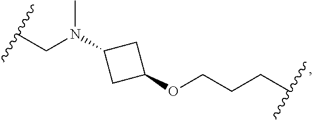 [SMILES: *CCCO[C@H]1C[C@H](N(C)C*)C1] | (293) |
| 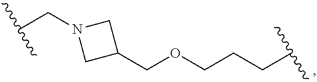 [SMILES: *CCCOCC1CN(C*)C1] | (294) |
| 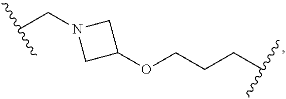 [SMILES: *CCCOC1CN(C*)C1] | (295) |
| 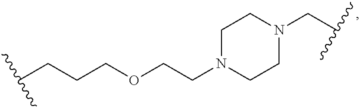 [SMILES: *CCCOCCN1CCN(C*)CC1] | (296) |
| 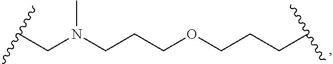 [SMILES: *CCCOCCCN(C)C*] | (297) |
| 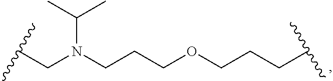 [SMILES: *CCCOCCCN(C*)C(C)C] | (298) |
| 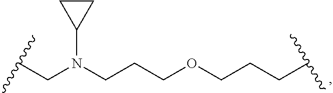 [SMILES: *CCCOCCCN(C*)C1CC1] | (299) |
| 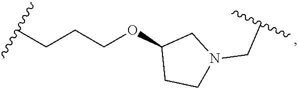 [SMILES: *CCCO[C@@H]1CCN(C*)C1] | (300) |
| 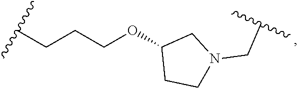 [SMILES: *CCCO[C@H]1CCN(C*)C1] | (301) |
| 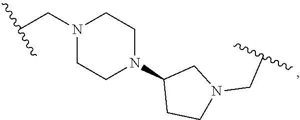 [SMILES: *CN1CCN([C@@H]2CCN(C*)C2)CC1] | (302) |
| 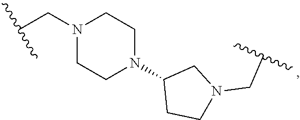 [SMILES: *CN1CCN([C@H]2CCN(C*)C2)CC1] | (303) |
| 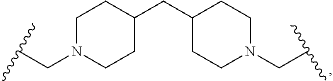 [SMILES: *CN1CCC(CC2CCN(C*)CC2)CC1] | (304) |
| 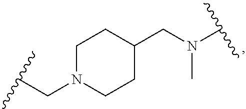 [SMILES: *CN1CCC(CN(*)C)CC1] | (305) |
| 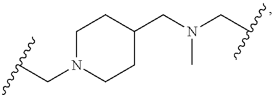 [SMILES: *CN(C)CC1CCN(C*)CC1] | (306) |
| 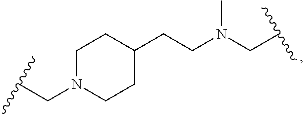 [SMILES: *CN(C)CCC1CCN(C*)CC1] | (307) |
| 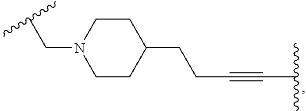 [SMILES: *C#CCCC1CCN(C*)CC1] | (308) |
| 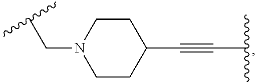 [SMILES: *C#CC1CCN(C*)CC1] | (309) |
| 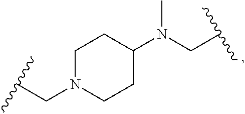 [SMILES: *CN1CCC(N(C)C*)CC1] | (310) |
| 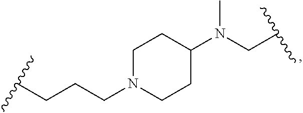 [SMILES: *CCCN1CCC(N(C)C*)CC1] | (311) |
| 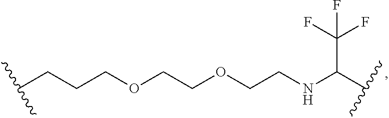 [SMILES: *CCCOCCOCCNC(*)C(F)(F)F] | (312) |
| 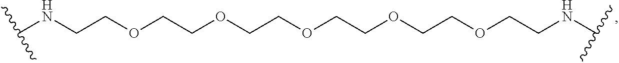 [SMILES: *NCCOCCOCCOCCOCCOCCN*] | (313) |
| 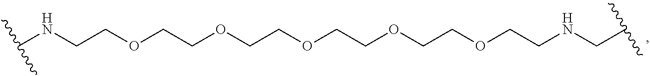 [SMILES: *CNCCOCCOCCOCCOCCOCCN*] | (314) |
| 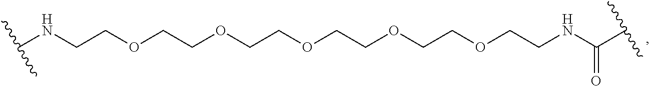 [SMILES: *NCCOCCOCCOCCOCCOCCNC(*)=O] | (315) |
| 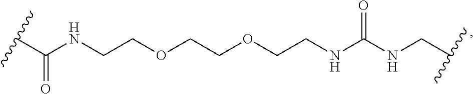 [SMILES: *CNC(=O)NCCOCCOCCNC(*)=O] | (316) |
| 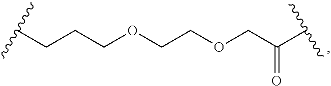 [SMILES: *CCCOCCOCC(*)=O] | (317) |
| 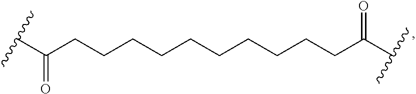 [SMILES: *C(=O)CCCCCCCCCCC(*)=O] | (318) |
| 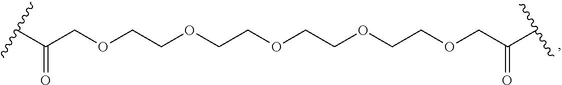 [SMILES: *C(=O)COCCOCCOCCOCCOCC(*)=O] | (319) |
| 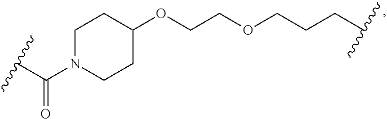 [SMILES: *CCCOCCOC1CCN(C(*)=O)CC1] | (320) |
| 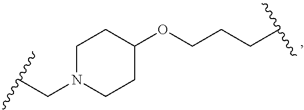 [SMILES: *CCCOC1CCN(C*)CC1] | (321) |
| 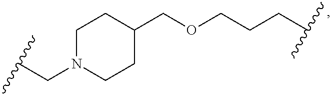 [SMILES: *CCCOCC1CCN(C*)CC1] | (322) |
| 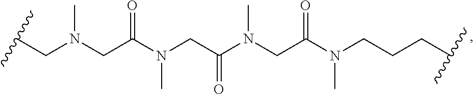 [SMILES: *CCCN(C)C(=O)CN(C)C(=O)CN(C)C(=O)CN(C)C*] | (323) |
| 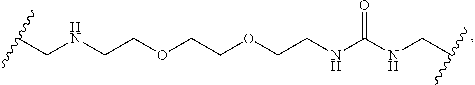 [SMILES: *CNCCOCCOCCNC(=O)NC*] | (324) |
| 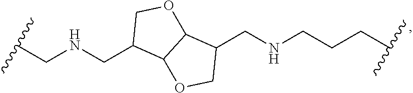 [SMILES: *CCCNCC1COC2C(CNC*)COC12] | (325) |
| 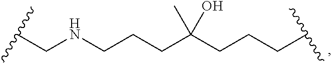 [SMILES: *CCCC(C)(O)CCCNC*] | (326) |
| 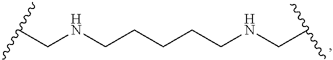 [SMILES: *CNCCCCCNC*] | (327) |
| 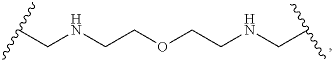 [SMILES: *CNCCOCCNC*] | (328) |
| 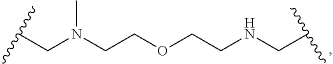 [SMILES: *CNCCOCCN(C)C*] | (329) |
| 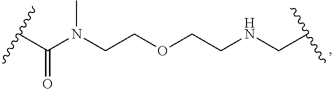 [SMILES: *CNCCOCCN(C)C(*)=O] | (330) |
| 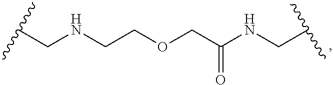 [SMILES: *CNCCOCC(=O)NC*] | (331) |
| 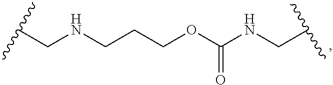 [SMILES: *CNCCCOC(=O)NC*] | (332) |
| 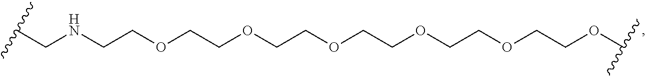 [SMILES: *CNCCOCCOCCOCCOCCOCCO*] | (333) |
| 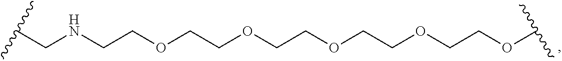 [SMILES: *CNCCOCCOCCOCCOCCO*] | (334) |
| 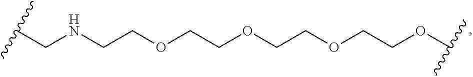 [SMILES: *CNCCOCCOCCOCCO*] | (335) |
| 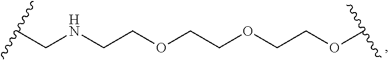 [SMILES: *CNCCOCCOCCO*] | (336) |
| 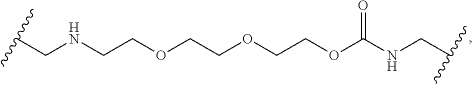 [SMILES: *CNCCOCCOCCOC(=O)NC*] | (337) |
| 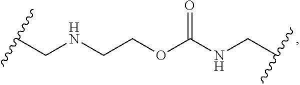 [SMILES: *CNCCOC(=O)NC*] | (338) |
| 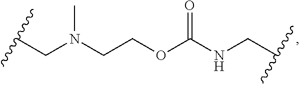 [SMILES: *CNC(=O)OCCN(C)C*] | (339) |
| 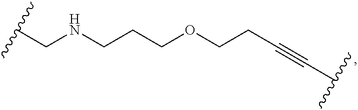 [SMILES: *C#CCCOCCCNC*] | (340) |
| 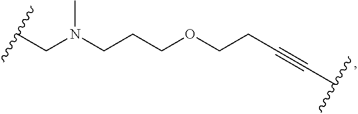 [SMILES: *C#CCCOCCCN(C)C*] | (341) |
| 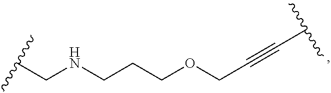 [SMILES: *C#CCOCCCNC*] | (342) |
| 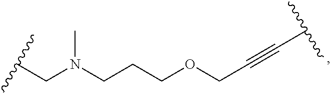 [SMILES: *C#CCOCCCN(C)C*] | (343) |
| 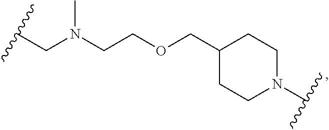 [SMILES: *CN(C)CCOCC1CCN(*)CC1] | (344) |
| 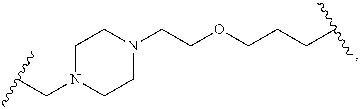 [SMILES: *CCCOCCN1CCN(C*)CC1] | (345) |
| 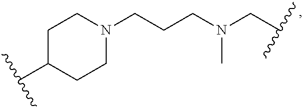 [SMILES: *CN(C)CCCN1CCC(*)CC1] | (346) |
| 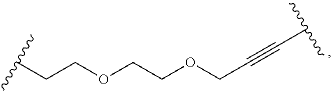 [SMILES: *C#CCOCCOCC*] | (347) |
| 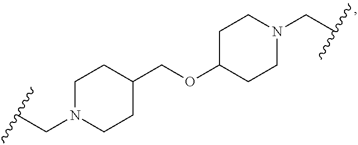 [SMILES: *CN1CCC(COC2CCN(C*)CC2)CC1] | (348) |
| 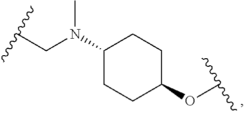 [SMILES: *CN(C)[C@H]1CC[C@H](O*)CC1] | (349) |
| 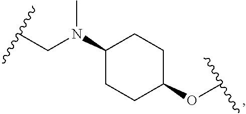 [SMILES: *CN(C)[C@H]1CC[C@@H](O*)CC1] | (350) |
| 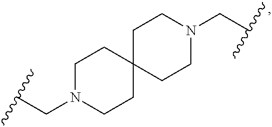 [SMILES: *CN1CCC2(CC1)CCN(C*)CC2] | (351) |
| 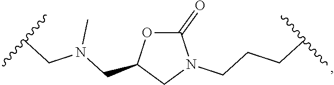 [SMILES: *CCCN1C[C@@H](CN(C)C*)OC1=O] | (352) |
| 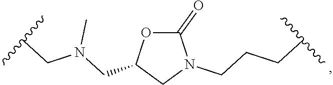 [SMILES: *CCCN1C[C@H](CN(C)C*)OC1=O] | (353) |
| 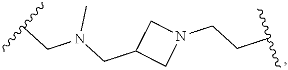 [SMILES: *CCN1CC(CN(C)C*)C1] | (354) |
| 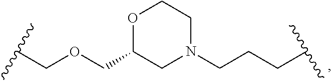 [SMILES: *CCCN1CCO[C@@H](COC*)C1] | (355) |
| 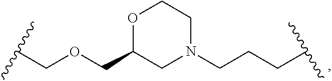 [SMILES: *CCCN1CCO[C@H](COC*)C1] | (356) |
| 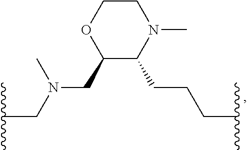 [SMILES: *CCC[C@@H]1[C@@H](CN(C)C*)OCCN1C] | (357) |
| 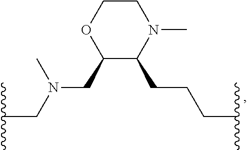 [SMILES: *CCC[C@H]1[C@@H](CN(C)C*)OCCN1C] | (358) |
| 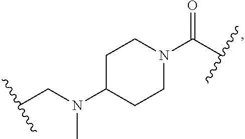 [SMILES: *CN(C)C1CCN(C(*)=O)CC1] | (359) |
| 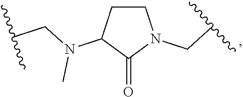 [SMILES: *CN1CCC(N(C)C*)C1=O] | (360) |
| 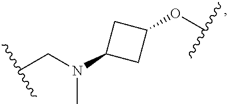 [SMILES: *CN(C)[C@H]1C[C@H](O*)C1] | (361) |
| 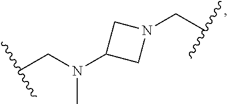 [SMILES: *CN1CC(N(C)C*)C1] | (362) |
| 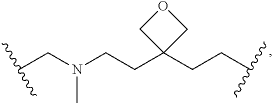 [SMILES: *CCC1(CCN(C)C*)COC1] | (363) |
| 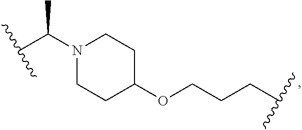 [SMILES: *CCCOC1CCN([C@@H](*)C)CC1] | (364) |
| 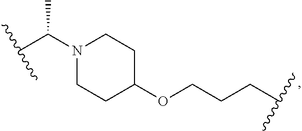 [SMILES: *CCCOC1CCN([C@H](*)C)CC1] | (365) |
| 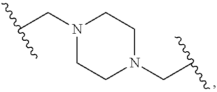 [SMILES: *CN1CCN(C*)CC1] | (366) |
| 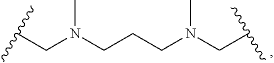 [SMILES: *CN(C)CCCN(C)C*] | (367) |
| 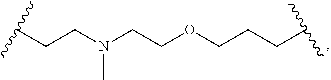 [SMILES: *CCCOCCN(C)CC*] | (368) |
| 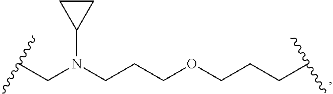 [SMILES: *CCCOCCCN(C*)C1CC1] | (369) |
| 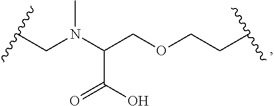 [SMILES: *CCOCC(C(=O)O)N(C)C*] | (370) |
| 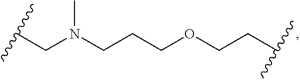 [SMILES: *CCOCCCN(C)C*] | (371) |
| 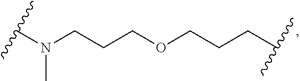 [SMILES: *CCCOCCCN(*)C] | (372) |
| 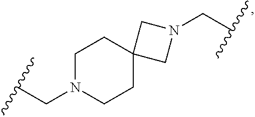 [SMILES: *CN1CCC2(CC1)CN(C*)C2] | (373) |
| 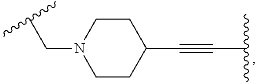 [SMILES: *C#CC1CCN(C*)CC1] | (374) |
| 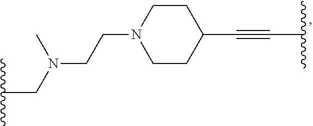 [SMILES: *C#CC1CCN(CCN(C)C*)CC1] | (375) |
| 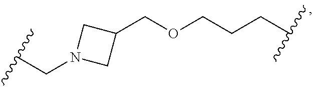 [SMILES: *CCCOCC1CN(C*)C1] | (376) |
| 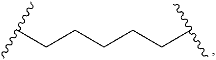 [SMILES: *CCCCC*] | (377) |
| 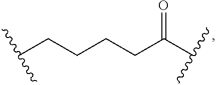 [SMILES: *CCCCC(*)=O] | (378) |
| 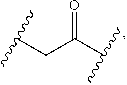 [SMILES: *CC(*)=O] | (379) |
| 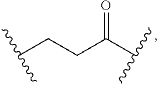 [SMILES: *CCC(*)=O] | (380) |
| 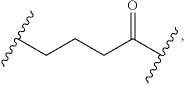 [SMILES: *CCCC(*)=O] | (381) |
| 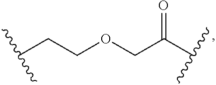 [SMILES: *CCOCC(*)=O] | (382) |
| 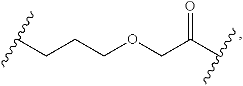 [SMILES: *CCCOCC(*)=O] | (383) |
| 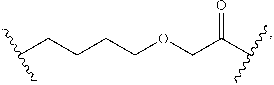 [SMILES: *CCCCOCC(*)=O] | (384) |
| 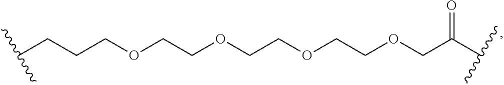 [SMILES: *CCCOCCOCCOCCOCC(*)=O] | (385) |
| 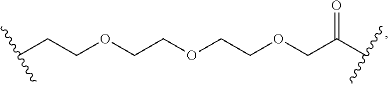 [SMILES: *CCOCCOCCOCC(*)=O] | (386) |
| 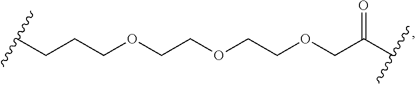 [SMILES: *CCCOCCOCCOCC(*)=O] | (387) |
| 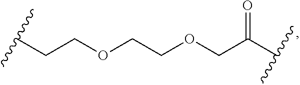 [SMILES: *CCOCCOCC(*)=O] | (388) |
| 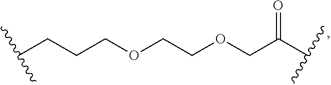 [SMILES: *CCCOCCOCC(*)=O] | (389) |
| 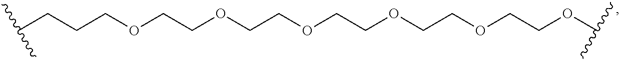 [SMILES: *CCCOCCOCCOCCOCCOCCO*] | (390) |
| 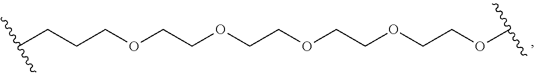 [SMILES: *CCCOCCOCCOCCOCCO*] | (391) |
| 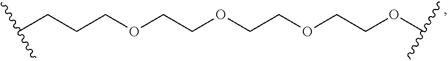 [SMILES: *CCCOCCOCCOCCO*] | (392) |
| 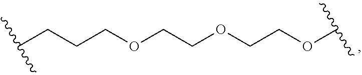 [SMILES: *CCCOCCOCCO*] | (393) |
| 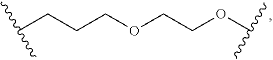 [SMILES: *CCCOCCO*] | (394) |
| 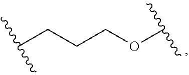 [SMILES: *CCCO*] | (395) |
| 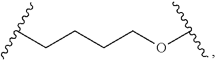 [SMILES: *CCCCO*] | (396) |
| 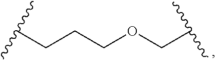 [SMILES: *CCCOC*] | (397) |
| 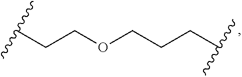 [SMILES: *CCCOCC*] | (398) |
| 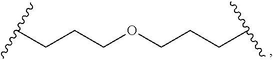 [SMILES: *CCCOCCC*] | (399) |
| 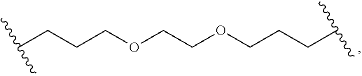 [SMILES: *CCCOCCOCCC*] | (400) |
| 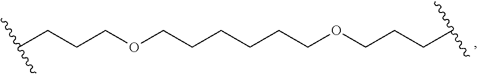 [SMILES: *CCCOCCCCCCOCCC*] | (401) |
| 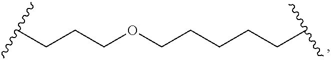 [SMILES: *CCCCCOCCC*] | (402) |
| 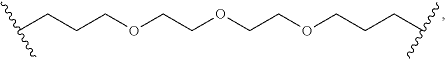 [SMILES: *CCCOCCOCCOCCC*] | (403) |
| 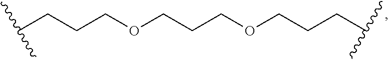 [SMILES: *CCCOCCCOCCC*] | (404) |
| 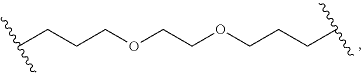 [SMILES: *CCCOCCOCCC*] | (405) |
| 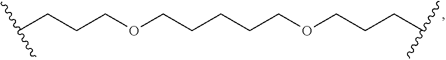 [SMILES: *CCCOCCCCCOCCC*] | (406) |
| 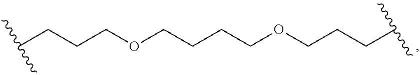 [SMILES: *CCCOCCCCOCCC*] | (407) |
| 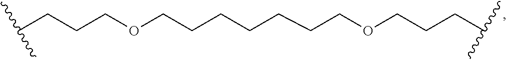 [SMILES: *CCCOCCCCCCCOCCC*] | (408) |
| 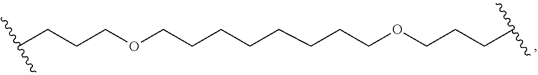 [SMILES: *CCCOCCCCCCCCOCCC*] | (409) |
| 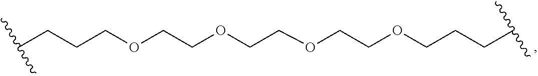 [SMILES: *CCCOCCOCCOCCOCCC*] | (410) |
| 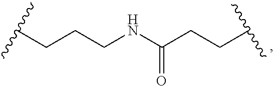 [SMILES: *CCCNC(=O)CC*] | (411) |
| 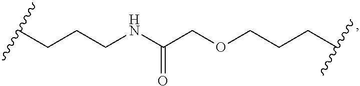 [SMILES: *CCCNC(=O)COCCC*] | (412) |
| 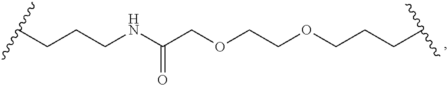 [SMILES: *CCCNC(=O)COCCOCCC*] | (413) |
| 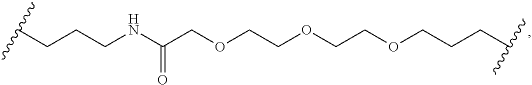 [SMILES: *CCCNC(=O)COCCOCCOCCC*] | (414) |
| 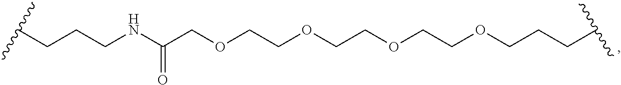 [SMILES: *CCCNC(=O)COCCOCCOCCOCCC*] | (415) |
| 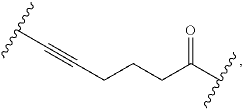 [SMILES: *C#CCCCC(*)=O] | (416) |
| 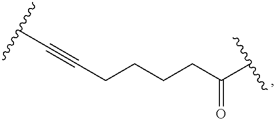 [SMILES: *C#CCCCCC(*)=O] | (417) |
| 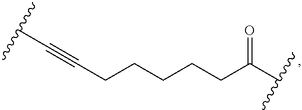 [SMILES: *C#CCCCCCC(*)=O] | (418) |
| 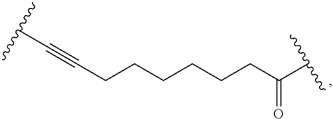 [SMILES: *C#CCCCCCCC(*)=O] | (419) |
| 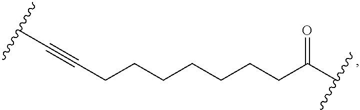 [SMILES: *C#CCCCCCCCC(*)=O] | (420) |
| 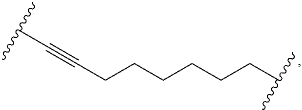 [SMILES: *C#CCCCCCC*] | (421) |
| 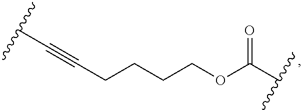 [SMILES: *C#CCCCCOC(*)=O] | (422) |
| 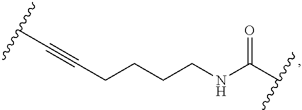 [SMILES: *C#CCCCCNC(*)=O] | (423) |
| 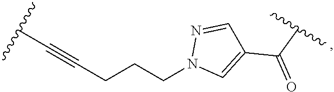 [SMILES: *C#CCCCn1cc(C(*)=O)cn1] | (424) |
| 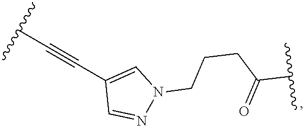 [SMILES: *C#Cc1cnn(CCCC(*)=O)c1] | (425) |
| 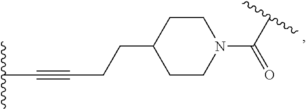 [SMILES: *C#CCCC1CCN(C(*)=O)CC1] | (426) |
| 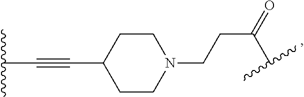 [SMILES: *C#CC1CCN(CCC(*)=O)CC1] | (427) |
| 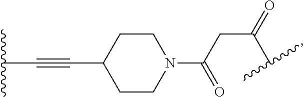 [SMILES: *C#CC1CCN(C(=O)CC(*)=O)CC1] | (428) |
| 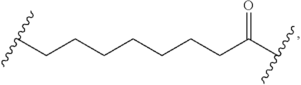 [SMILES: *CCCCCCCC(*)=O] | (429) |
| 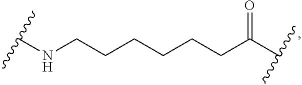 [SMILES: *NCCCCCCC(*)=O] | (430) |
| 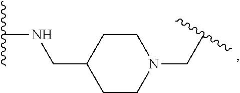 [SMILES: *CN1CCC(CN*)CC1] | (431) |
| 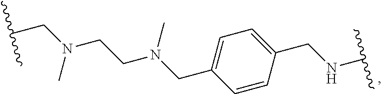 [SMILES: *CN(C)CCN(C)Cc1ccc(CN*)cc1] | (432) |
| 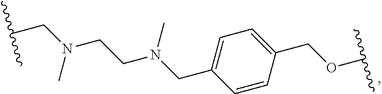 [SMILES: *CN(C)CCN(C)Cc1ccc(CO*)cc1] | (433) |
| 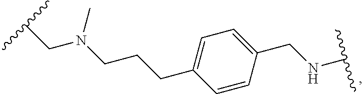 [SMILES: *CN(C)CCCc1ccc(CN*)cc1] | (434) |
| 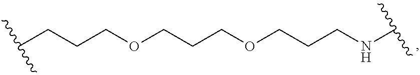 [SMILES: *CCCOCCCOCCCN*] | (435) |
| 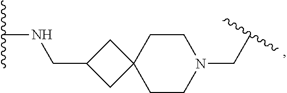 [SMILES: *CN1CCC2(CC1)CC(CN*)C2] | (436) |
| 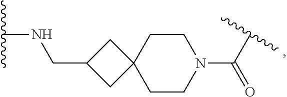 [SMILES: *NCC1CC2(CCN(C(*)=O)CC2)C1] | (437) |
| 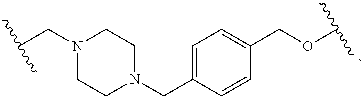 [SMILES: *CN1CCN(Cc2ccc(CO*)cc2)CC1] | (438) |
| 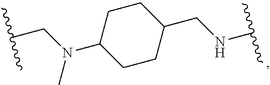 [SMILES: *CN(C)C1CCC(CN*)CC1] | (438) |
| 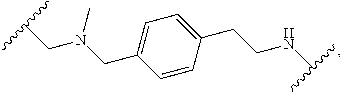 [SMILES: *CN(C)Cc1ccc(CCN*)cc1] | (439) |
| 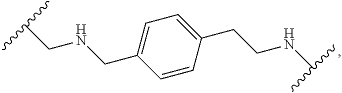 [SMILES: *CNCc1ccc(CCN*)cc1] | (440) |
| 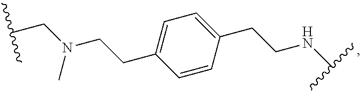 [SMILES: *CN(C)CCc1ccc(CCN*)cc1] | (441) |
| 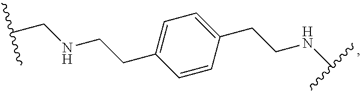 [SMILES: *CNCCc1ccc(CCN*)cc1] | (442) |
| 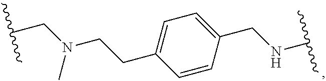 [SMILES: *CN(C)CCc1ccc(CN*)cc1] | (443) |
| 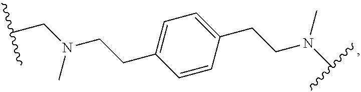 [SMILES: *CN(C)CCc1ccc(CCN(*)C)cc1] | (444) |
| 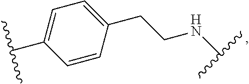 [SMILES: *NCCc1ccc(*)cc1] | (445) |
| 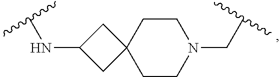 [SMILES: *CN1CCC2(CC1)CC(N*)C2] | (446) |
| 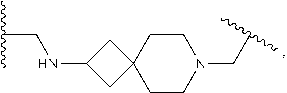 [SMILES: *CNC1CC2(CCN(C*)CC2)C1] | (447) |
| 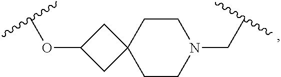 [SMILES: *CN1CCC2(CC1)CC(O*)C2] | (448) |
| 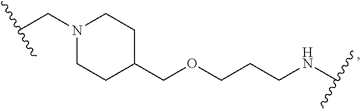 [SMILES: *CN1CCC(COCCCN*)CC1] | (449) |
| 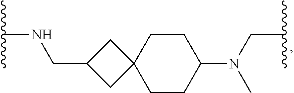 [SMILES: *CN(C)C1CCC2(CC1)CC(CN*)C2] | (450) |
| 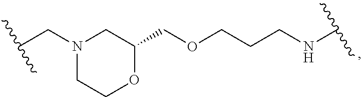 [SMILES: *CN1CCO[C@@H](COCCCN*)C1] | (451) |
| 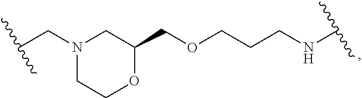 [SMILES: *CN1CCO[C@H](COCCCN*)C1] | (452) |
| 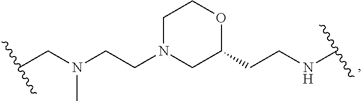 [SMILES: *CN(C)CCN1CCO[C@H](CCN*)C1] | (453) |
| 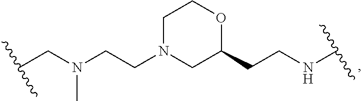 [SMILES: *CN(C)CCN1CCO[C@@H](CCN*)C1] | (454) |
| 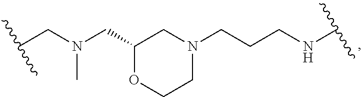 [SMILES: *CN(C)C[C@@H]1CN(CCCN*)CCO1] | (455) |
| 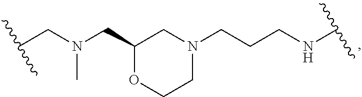 [SMILES: *CN(C)C[C@H]1CN(CCCN*)CCO1] | (456) |
| 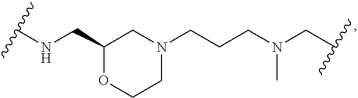 [SMILES: *CN(C)CCCN1CCO[C@@H](CN*)C1] | (457) |
| 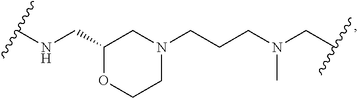 [SMILES: *CN(C)CCCN1CCO[C@H](CN*)C1] | (458) |
| 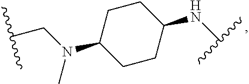 [SMILES: *CN(C)[C@H]1CC[C@@H](N*)CC1] | (459) |
| 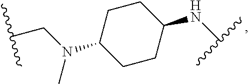 [SMILES: *CN(C)[C@H]1CC[C@H](N*)CC1] | (460) |
| 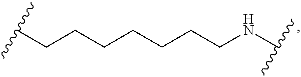 [SMILES: *CCCCCCCN*] | (461) |
| 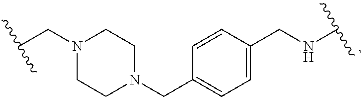 [SMILES: *CN1CCN(Cc2ccc(CN*)cc2)CC1] | (462) |
| 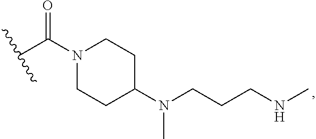 [SMILES: *C(=O)N1CCC(N(C)CCCNC)CC1] | (463) |
| 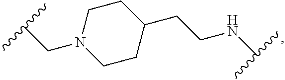 [SMILES: *CN1CCC(CCN*)CC1] | (464) |
| 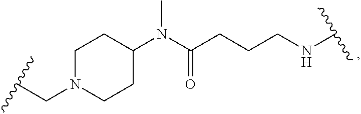 [SMILES: *CN1CCC(N(C)C(=O)CCCN*)CC1] | (465) |
| 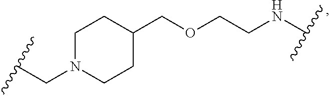 [SMILES: *CN1CCC(COCCN*)CC1] | (466) |
| 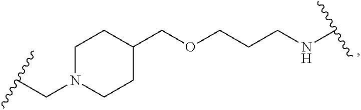 [SMILES: *CN1CCC(COCCCN*)CC1] | (467) |
| 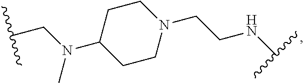 [SMILES: *CN(C)C1CCN(CCN*)CC1] | (468) |
| 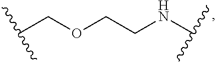 [SMILES: *COCCN*] | (469) |
| 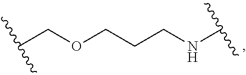 [SMILES: *COCCCN*] | (470) |
| 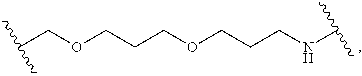 [SMILES: *COCCCOCCCN*] | (471) |
| 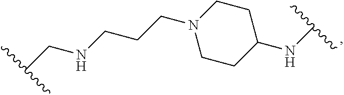 [SMILES: *CNCCCN1CCC(N*)CC1] | (472) |
| 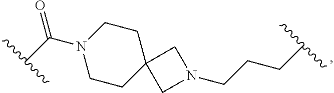 [SMILES: *CCCN1CC2(CCN(C(*)=O)CC2)C1] | (473) |
| 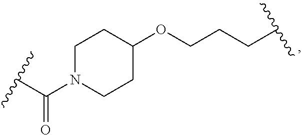 [SMILES: *CCCOC1CCN(C(*)=O)CC1] | (474) |
| 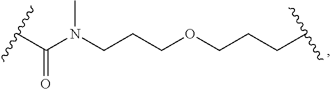 [SMILES: *CCCOCCCN(C)C(*)=O] | (475) |
| 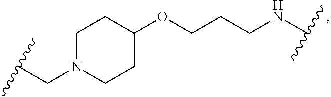 [SMILES: *CN1CCC(OCCCN*)CC1] | (475) |
| 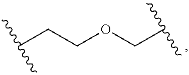 [SMILES: *CCOC*] | (476) |
| 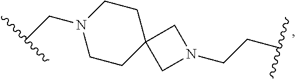 [SMILES: *CCN1CC2(CCN(C*)CC2)C1] | (477) |
| 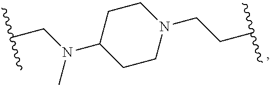 [SMILES: *CCN1CCC(N(C)C*)CC1] | (478) |
| 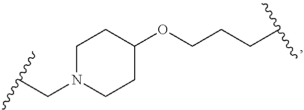 [SMILES: *CCCOC1CCN(C*)CC1] | (479) |
| 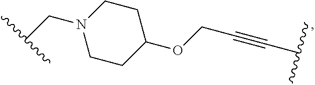 [SMILES: *C#CCOC1CCN(C*)CC1] | (480) |
| 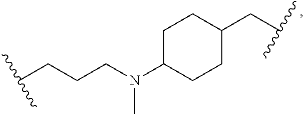 [SMILES: *CCCN(C)C1CCC(C*)CC1] | (481) |
| 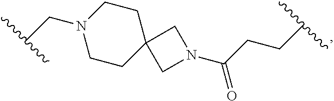 [SMILES: *CCC(=O)N1CC2(CCN(C*)CC2)C1] | (482) |
| 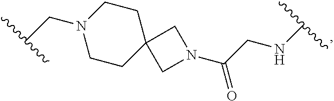 [SMILES: *CN1CCC2(CC1)CN(C(=O)CN*)C2] | (483) |
| 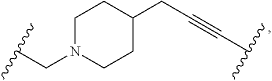 [SMILES: *C#CCC1CCN(C*)CC1] | (484) |
| 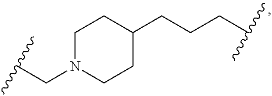 [SMILES: *CCCC1CCN(C*)CC1] | (485) |
| 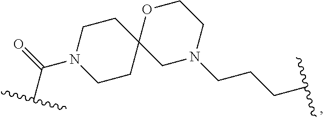 [SMILES: *CCCN1CCOC2(CCN(C(*)=O)CC2)C1] | (486) |
| 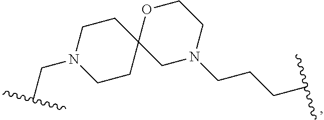 [SMILES: *CCCN1CCOC2(CCN(C*)CC2)C1] | (487) |
| 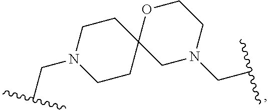 [SMILES: *CN1CCC2(CC1)CN(C*)CCO2] | (488) |
| 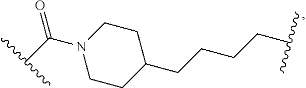 [SMILES: *CCCCC1CCN(C(*)=O)CC1] | (489) |
| 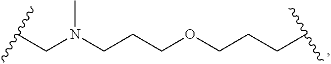 [SMILES: *CCCOCCCN(C)C*] | (490) |
| 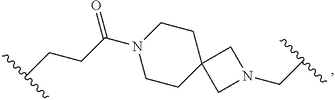 [SMILES: *CCC(=O)N1CCC2(CC1)CN(C*)C2] | (491) |
| 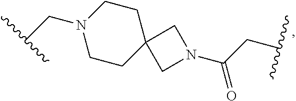 [SMILES: *CC(=O)N1CC2(CCN(C*)CC2)C1] | (492) |
| 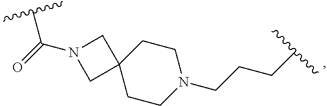 [SMILES: *CCCN1CCC2(CC1)CN(C(*)=O)C2] | (493) |
| 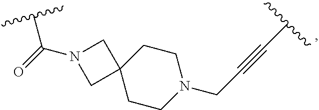 [SMILES: *C#CCN1CCC2(CC1)CN(C(*)=O)C2] | (494) |
| 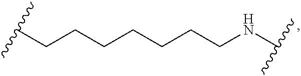 [SMILES: *CCCCCCCN*] | (495) |
| 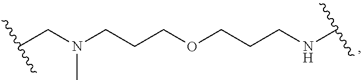 [SMILES: *CN(C)CCCOCCCN*] | (496) |
| 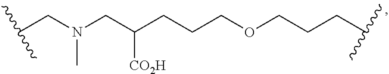 [SMILES: *CCCOCCCC(CN(C)C*)C(=O)O] | (497) |
| 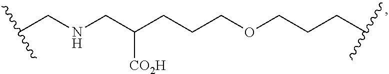 [SMILES: *CCCOCCCC(CNC*)C(=O)O] | (498) |
| 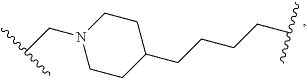 [SMILES: *CCCCC1CCN(C*)CC1] | (499) |
| 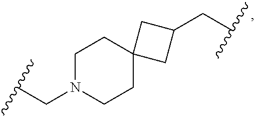 [SMILES: *CC1CC2(CCN(C*)CC2)C1] | (500) |
| 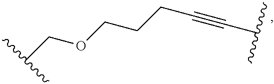 [SMILES: *C#CCCCOC*] | (501) |
| 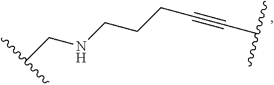 [SMILES: *C#CCCCNC*] | (502) |
| 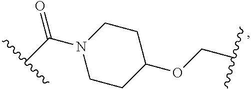 [SMILES: *COC1CCN(C(*)=O)CC1] | (503) |
| 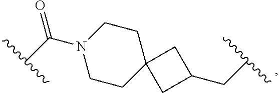 [SMILES: *CC1CC2(CCN(C(*)=O)CC2)C1] | (504) |
| 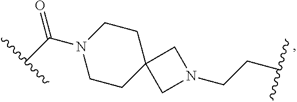 [SMILES: *CCN1CC2(CCN(C(*)=O)CC2)C1] | (505) |
| 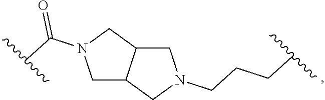 [SMILES: *CCCN1CC2CN(C(*)=O)CC2C1] | (506) |
| 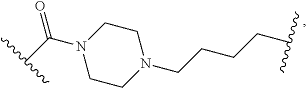 [SMILES: *CCCCN1CCN(C(*)=O)CC1] | (507) |
| 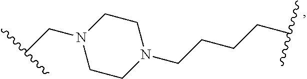 [SMILES: *CCCCN1CCN(C*)CC1] | (508) |
| 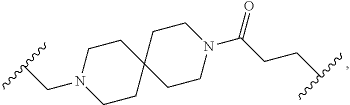 [SMILES: *CCC(=O)N1CCC2(CCN(C*)CC2)CC1] | (509) |
| 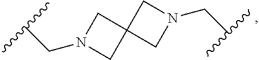 [SMILES: *CN1CC2(C1)CN(C*)C2] | (510) |
| 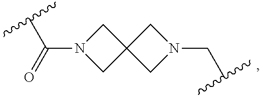 [SMILES: *CN1CC2(C1)CN(C(*)=O)C2] | (511) |
| 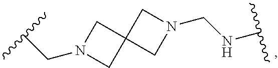 [SMILES: *CN1CC2(C1)CN(CN*)C2] | (512) |
| 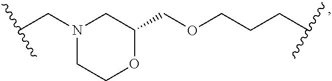 [SMILES: *CCCOC[C@H]1CN(C*)CCO1] | (513) |
| 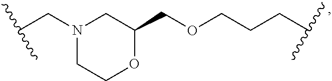 [SMILES: *CCCOC[C@@H]1CN(C*)CCO1] | (514) |
| 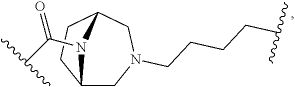 [SMILES: *CCCCN1C[C@H]2CC[C@@H](C1)N2C(*)=O] | (515) |
| 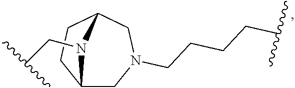 [SMILES: *CCCCN1C[C@H]2CC[C@@H](C1)N2C*] | (516) |
| 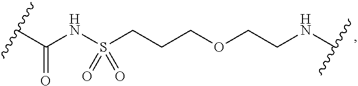 [SMILES: *NCCOCCCS(=O)(=O)NC(*)=O] | (517) |
| 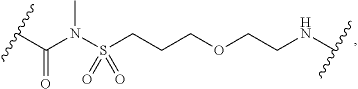 [SMILES: *NCCOCCCS(=O)(=O)N(C)C(*)=O] | (518) |
| 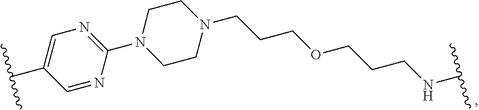 [SMILES: *NCCCOCCCN1CCN(c2ncc(*)cn2)CC1] | (519) |
| 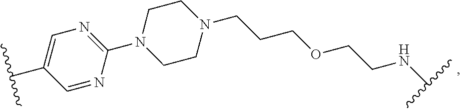 [SMILES: *NCCOCCCN1CCN(c2ncc(*)cn2)CC1] | (520) |
| 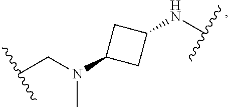 [SMILES: *CN(C)[C@H]1C[C@H](N*)C1] | (521) |
| 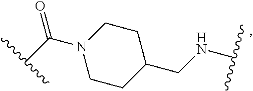 [SMILES: *NCC1CCN(C(*)=O)CC1] | (522) |
| 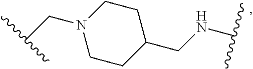 [SMILES: *CN1CCC(CN*)CC1] | (523) |
| 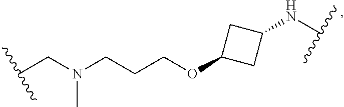 [SMILES: *CN(C)CCCO[C@H]1C[C@H](N*)C1] | (524) |
| 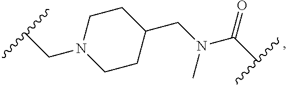 [SMILES: *CN1CCC(CN(C)C(*)=O)CC1] | (525) |
| 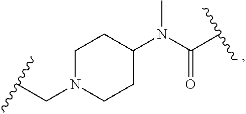 [SMILES: *CN1CCC(N(C)C(*)=O)CC1] | (526) |
| 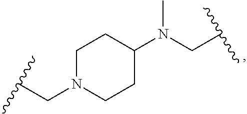 [SMILES: *CN1CCC(N(C)C*)CC1] | (527) |
| 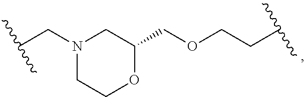 [SMILES: *CCOC[C@H]1CN(C*)CCO1] | (528) |
| 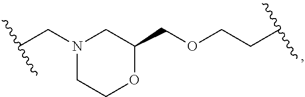 [SMILES: *CCOC[C@@H]1CN(C*)CCO1] | (529) |
| 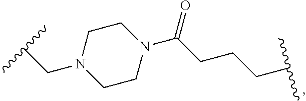 [SMILES: *CCCC(=O)N1CCN(C*)CC1] | (530) |
| 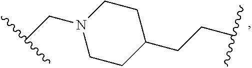 [SMILES: *CCC1CCN(C*)CC1] | (531) |
| 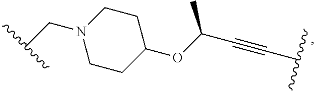 [SMILES: *C#C[C@H](C)OC1CCN(C*)CC1] | (532) |
| 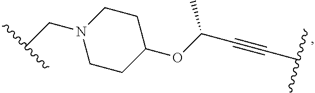 [SMILES: *C#C[C@@H](C)OC1CCN(C*)CC1] | (533) |
| 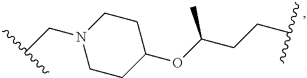 [SMILES: *CC[C@H](C)OC1CCN(C*)CC1] | (534) |
| 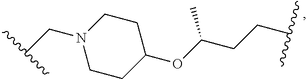 [SMILES: *CC[C@@H](C)OC1CCN(C*)CC1] | (535) |
| 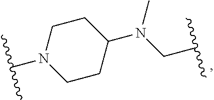 [SMILES: *CN(C)C1CCN(*)CC1] | (536) |
| 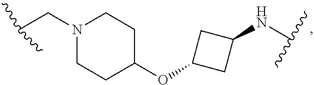 [SMILES: *CN1CCC(O[C@H]2C[C@H](N*)C2)CC1] | (537) |
| 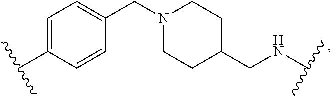 [SMILES: *NCC1CCN(Cc2ccc(*)cc2)CC1] | (538) |
| 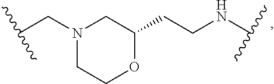 [SMILES: *CN1CCO[C@@H](CCN*)C1] | (539) |
| 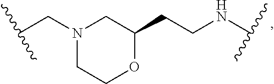 [SMILES: *CN1CCO[C@H](CCN*)C1] | (540) |
| 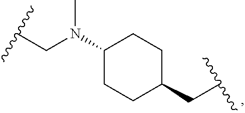 [SMILES: *CN(C)[C@H]1CC[C@H](C*)CC1] | (541) |
| 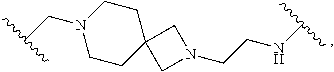 [SMILES: *CN1CCC2(CC1)CN(CCN*)C2] | (542) |
| 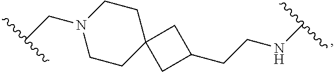 [SMILES: *CN1CCC2(CC1)CC(CCN*)C2] | (543) |
| 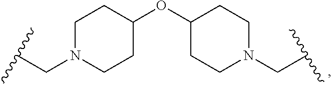 [SMILES: *CN1CCC(OC2CCN(C*)CC2)CC1] | (544) |
| 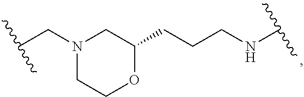 [SMILES: *CN1CCO[C@@H](CCCN*)C1] | (545) |
| 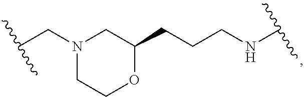 [SMILES: *CN1CCO[C@H](CCCN*)C1] | (546) |
| 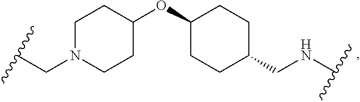 [SMILES: *CN1CCC(O[C@H]2CC[C@H](CN*)CC2)CC1] | (547) |
| 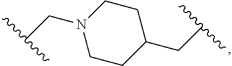 [SMILES: *CC1CCN(C*)CC1] | (548) |
| 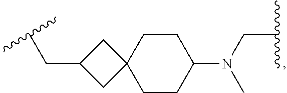 [SMILES: *CC1CC2(CCC(N(C)C*)CC2)C1] | (549) |
| 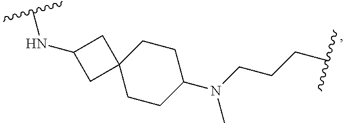 [SMILES: *CCCN(C)C1CCC2(CC1)CC(N*)C2] | (550) |
| 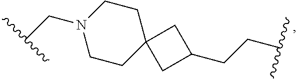 [SMILES: *CCC1CC2(CCN(C*)CC2)C1] | (551) |
| 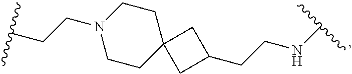 [SMILES: *CCN1CCC2(CC1)CC(CCN*)C2] | (552) |
| 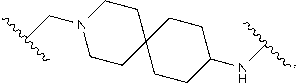 [SMILES: *CN1CCC2(CCC(N*)CC2)CC1] | (553) |
| 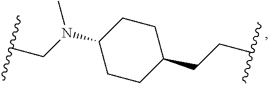 [SMILES: *CC[C@H]1CC[C@H](N(C)C*)CC1] | (554) |
| 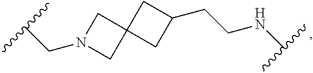 [SMILES: *CN1CC2(CC(CCN*)C2)C1] | (555) |
| 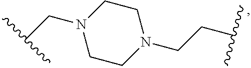 [SMILES: *CCN1CCN(C*)CC1] | (556) |
| 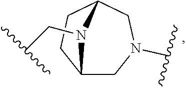 [SMILES: *CN1[C@@H]2CC[C@H]1CN(*)C2] | (557) |
| 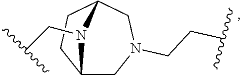 [SMILES: *CCN1C[C@H]2CC[C@@H](C1)N2C*] | (558) |
| 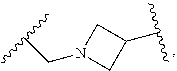 [SMILES: *CN1CC(*)C1] | (559) |
| 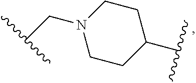 [SMILES: *CN1CCC(*)CC1] | (560) |
| 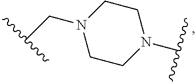 [SMILES: *CN1CCN(*)CC1] | (561) |
| 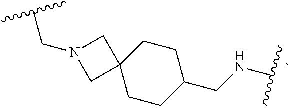 [SMILES: *CN1CC2(CCC(CN*)CC2)C1] | (562) |
| 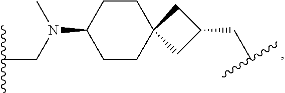 | (563) |
| 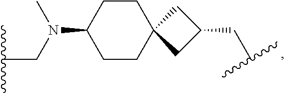 | (564) |
| 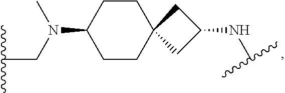 | (565) |
| 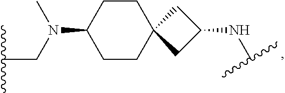 | (566) |
| 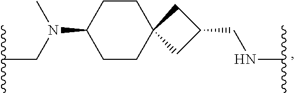 | (567) |
| 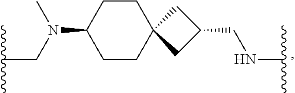 | (568) |
| 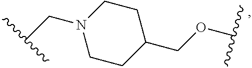 [SMILES: *CN1CCC(CO*)CC1] | (569) |
| 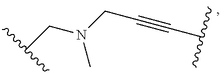 [SMILES: *C#CCN(C)C*] | (570) |
| 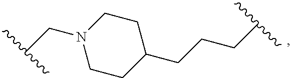 [SMILES: *CCCC1CCN(C*)CC1] | (571) |
| 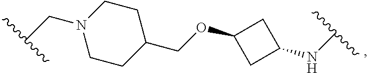 [SMILES: *CN1CCC(CO[C@H]2C[C@H](N*)C2)CC1] | (572) |
| 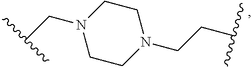 [SMILES: *CCN1CCN(C*)CC1] | (573) |
| 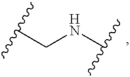 [SMILES: *CN*] | (574) |
| 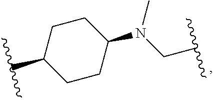 [SMILES: *CN(C)[C@H]1CC[C@@H](*)CC1] | (575) |
| 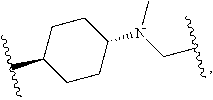 [SMILES: *CN(C)[C@H]1CC[C@H](*)CC1] | (576) |
| 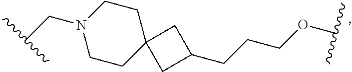 [SMILES: *CN1CCC2(CC1)CC(CCCO*)C2] | (577) |
| 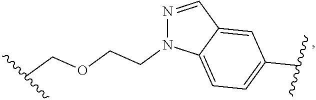 [SMILES: *COCCn1ncc2cc(*)ccc21] | (578) |
| 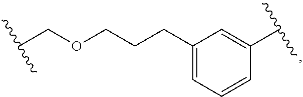 [SMILES: *COCCCc1cccc(*)c1] | (579) |
| 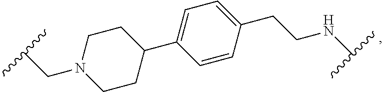 [SMILES: *CN1CCC(c2ccc(CCN*)cc2)CC1] | (580) |
| 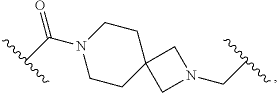 [SMILES: *CN1CC2(CCN(C(*)=O)CC2)C1] | (581) |
| 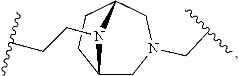 [SMILES: *CCN1[C@@H]2CC[C@H]1CN(C*)C2] | (582) |
| 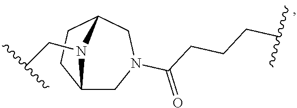 [SMILES: *CCCC(=O)N1C[C@H]2CC[C@@H](C1)N2C*] | (583) |
| 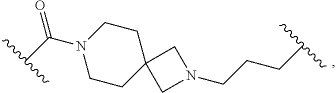 [SMILES: *CCCN1CC2(CCN(C(*)=O)CC2)C1] | (584) |
| 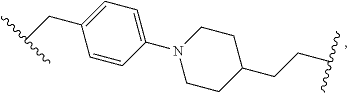 [SMILES: *CCC1CCN(c2ccc(C*)cc2)CC1] | (585) |
| 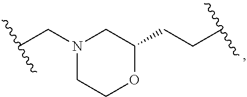 [SMILES: *CC[C@H]1CN(C*)CCO1] | (586) |
| 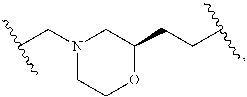 [SMILES: *CC[C@@H]1CN(C*)CCO1] | (587) |
| 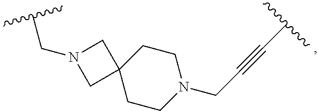 [SMILES: *C#CCN1CCC2(CC1)CN(C*)C2] | (588) |
| 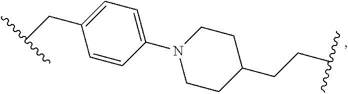 [SMILES: *CCC1CCN(c2ccc(C*)cc2)CC1] | (589) |
| 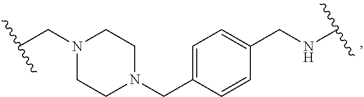 [SMILES: *CN1CCN(Cc2ccc(CN*)cc2)CC1] | (590) |
| 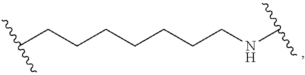 [SMILES: *CCCCCCCN*] | (591) |
| 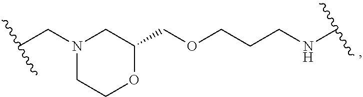 [SMILES: *CN1CCO[C@@H](COCCCN*)C1] | (592) |
| 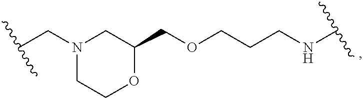 [SMILES: *CN1CCO[C@H](COCCCN*)C1] | (593) |
| 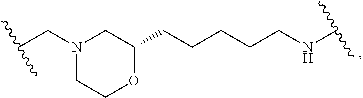 [SMILES: *CN1CCO[C@@H](CCCCCN*)C1] | (594) |
| 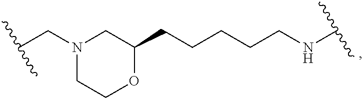 [SMILES: *CN1CCO[C@H](CCCCCN*)C1] | (595) |
| 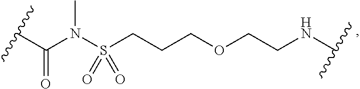 [SMILES: *NCCOCCCS(=O)(=O)N(C)C(*)=O] | (596) |
| 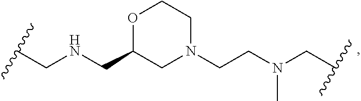 [SMILES: *CNC[C@@H]1CN(CCN(C)C*)CCO1] | (597) |
| 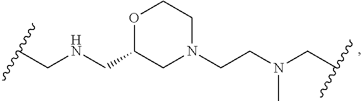 [SMILES: *CNC[C@H]1CN(CCN(C)C*)CCO1] | (598) |
| 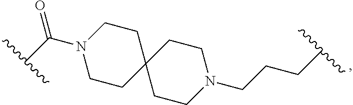 [SMILES: *CCCN1CCC2(CC1)CCN(C(*)=O)CC2] | (599) |
| 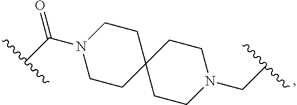 [SMILES: *CN1CCC2(CC1)CCN(C(*)=O)CC2] | (600) |
| 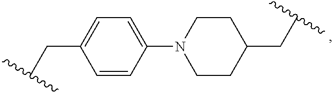 [SMILES: *Cc1ccc(N2CCC(C*)CC2)cc1] | (601) |
| 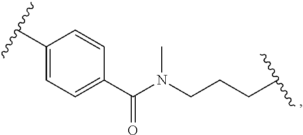 [SMILES: *CCCN(C)C(=O)c1ccc(*)cc1] | (602) |
| 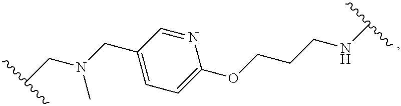 [SMILES: *CN(C)Cc1ccc(OCCCN*)nc1] | (603) |
| 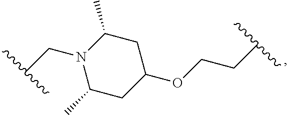 [SMILES: *CCOC1C[C@@H](C)N(C*)[C@@H](C)C1] | (604) |
| 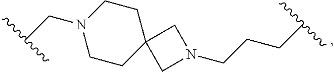 [SMILES: *CCCN1CC2(CCN(C*)CC2)C1] | (605) |
| 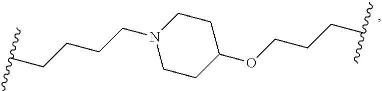 [SMILES: *CCCCN1CCC(OCCC*)CC1] | (606) |
| 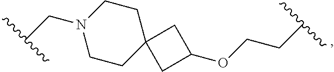 [SMILES: *CCOC1CC2(CCN(C*)CC2)C1] | (607) |
| 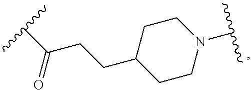 [SMILES: *C(=O)CCC1CCN(*)CC1] | (608) |
| 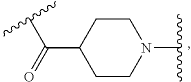 [SMILES: *C(=O)C1CCN(*)CC1] | (609) |
| 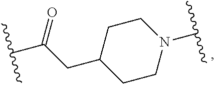 [SMILES: *C(=O)CC1CCN(*)CC1] | (610) |
| 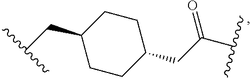 [SMILES: *C[C@H]1CC[C@H](CC(*)=O)CC1] | (611) |
| 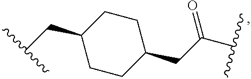 [SMILES: *C[C@H]1CC[C@@H](CC(*)=O)CC1] | (612) |
| 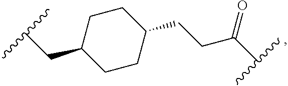 [SMILES: *C[C@H]1CC[C@H](CCC(*)=O)CC1] | (613) |
| 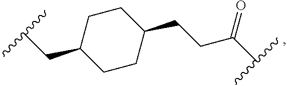 [SMILES: *C[C@H]1CC[C@@H](CCC(*)=O)CC1] | (614) |
| 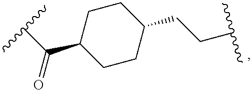 [SMILES: *CC[C@H]1CC[C@H](C(*)=O)CC1] | (615) |
| 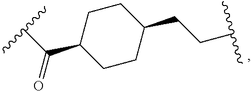 [SMILES: *CC[C@H]1CC[C@@H](C(*)=O)CC1] | (616) |
| 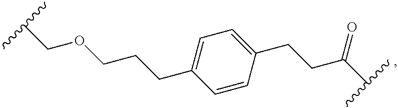 [SMILES: *COCCCc1ccc(CCC(*)=O)cc1] | (617) |
| 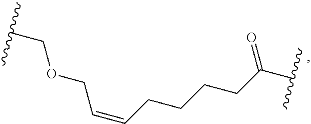 [SMILES: *COC/C=C\CCCCC(*)=O] | (618) |
| 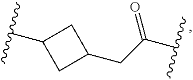 [SMILES: *C(=O)CC1CC(*)C1] | (619) |
| 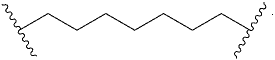 [SMILES: *CCCCCCC*] | (620) |
| 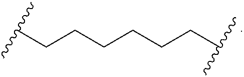 [SMILES: *CCCCCC*] | (621) |
| 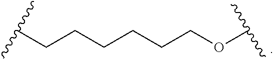 [SMILES: *CCCCCCO*] | (622) |
| 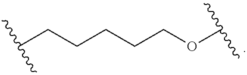 [SMILES: *CCCCCO*] | (623) |
| 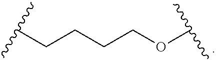 [SMILES: *CCCCO*] | (624) |
| 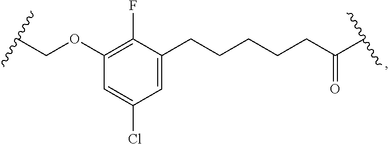 [SMILES: *COc1cc(Cl)cc(CCCCCC(*)=O)c1F] | (625) |
| 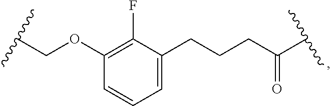 [SMILES: *COc1cccc(CCCC(*)=O)c1F] | (626) |
| 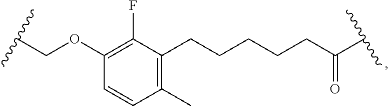 [SMILES: *COc1ccc(C)c(CCCCCC(*)=O)c1F] | (627) |
| 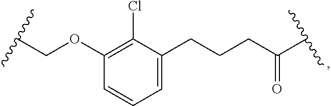 [SMILES: *COc1cccc(CCCC(*)=O)c1Cl] | (628) |
| 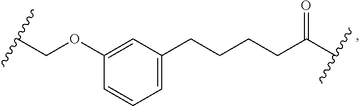 [SMILES: *COc1cccc(CCCCC(*)=O)c1] | (629) |
| 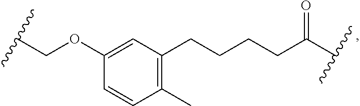 [SMILES: *COc1ccc(C)c(CCCCC(*)=O)c1] | (630) |
| 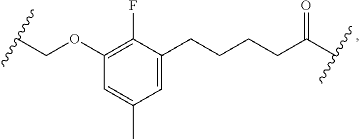 [SMILES: *COc1cc(C)cc(CCCCC(*)=O)c1F] | (631) |
| 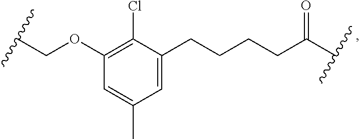 [SMILES: *COc1cc(C)cc(CCCCC(*)=O)c1Cl] | (632) |
| 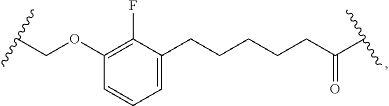 [SMILES: *COc1cccc(CCCCCC(*)=O)c1F] | (633) |
| 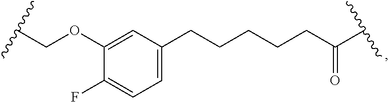 [SMILES: *COc1cc(CCCCCC(*)=O)ccc1F] | (634) |
| 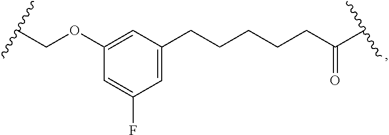 [SMILES: *COc1cc(F)cc(CCCCCC(*)=O)c1] | (635) |
| 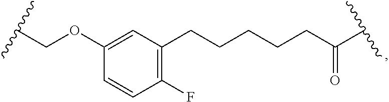 [SMILES: *COc1ccc(F)c(CCCCCC(*)=O)c1] | (636) |
| 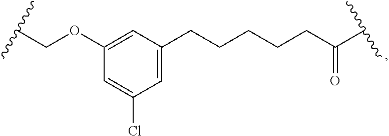 [SMILES: *COc1cc(Cl)cc(CCCCCC(*)=O)c1] | (637) |
| 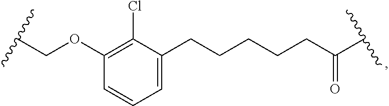 [SMILES: *COc1cccc(CCCCCC(*)=O)c1Cl] | (638) |
| 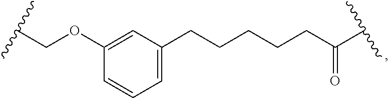 [SMILES: *COc1cccc(CCCCCC(*)=O)c1] | (639) |
| 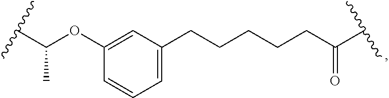 [SMILES: *C(=O)CCCCCc1cccc(O[C@@H](*)C)c1] | (640) |
| 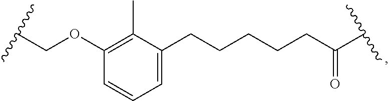 [SMILES: *COc1cccc(CCCCCC(*)=O)c1C] | (641) |
| 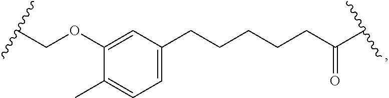 [SMILES: *COc1cc(CCCCCC(*)=O)ccc1C] | (642) |
| 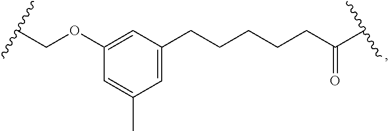 [SMILES: *COc1cc(C)cc(CCCCCC(*)=O)c1] | (643) |
| 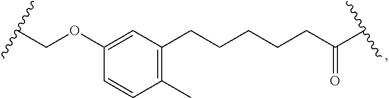 [SMILES: *COc1ccc(C)c(CCCCCC(*)=O)c1] | (644) |
| 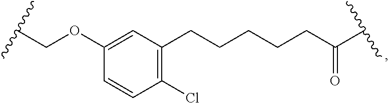 [SMILES: *COc1ccc(Cl)c(CCCCCC(*)=O)c1] | (645) |
| 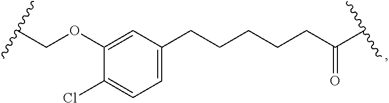 [SMILES: *COc1cc(CCCCCC(*)=O)ccc1Cl] | (646) |
| 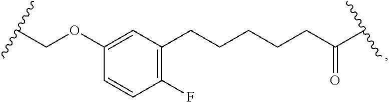 [SMILES: *COc1ccc(F)c(CCCCCC(*)=O)c1] | (647) |
| 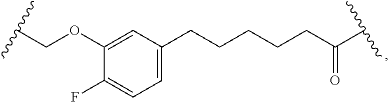 [SMILES: *COc1cc(CCCCCC(*)=O)ccc1F] | (648) |
| 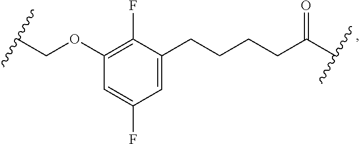 [SMILES: *COc1cc(F)cc(CCCCC(*)=O)c1F] | (649) |
| 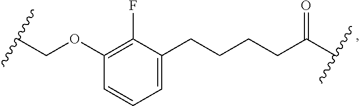 [SMILES: *COc1cccc(CCCCC(*)=O)c1F] | (650) |
| 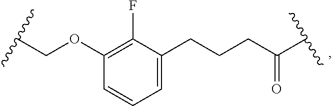 [SMILES: *COc1cccc(CCCC(*)=O)c1F] | (651) |
| 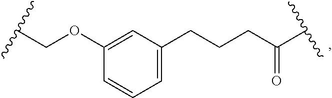 [SMILES: *COc1cccc(CCCC(*)=O)c1] | (652) |
| 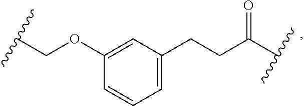 [SMILES: *COc1cccc(CCC(*)=O)c1] | (653) |
| 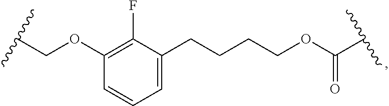 [SMILES: *COc1cccc(CCCCOC(*)=O)c1F] | (654) |
| 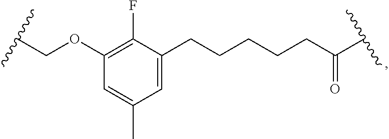 [SMILES: *COc1cc(C)cc(CCCCCC(*)=O)c1F] | (655) |
| 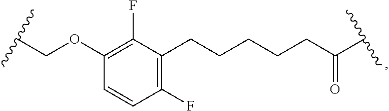 [SMILES: *COc1ccc(F)c(CCCCCC(*)=O)c1F] | (656) |
| 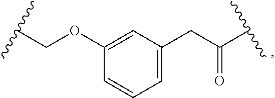 [SMILES: *COc1cccc(CC(*)=O)c1] | (657) |
| 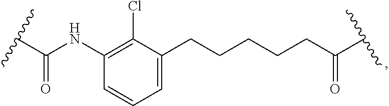 [SMILES: *C(=O)CCCCCc1cccc(NC(*)=O)c1Cl] | (658) |
| 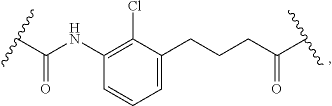 [SMILES: *C(=O)CCCc1cccc(NC(*)=O)c1Cl] | (659) |
| 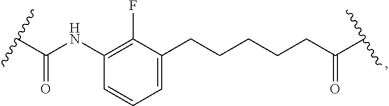 [SMILES: *C(=O)CCCCCc1cccc(NC(*)=O)c1F] | (660) |
| 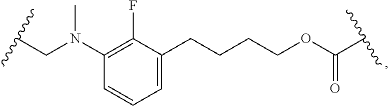 [SMILES: *CN(C)c1cccc(CCCCOC(*)=O)c1F] | (661) |
| 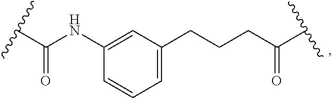 [SMILES: *C(=O)CCCc1cccc(NC(*)=O)c1] | (662) |
| 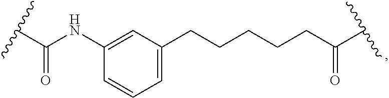 [SMILES: *C(=O)CCCCCc1cccc(NC(*)=O)c1] | (663) |
| 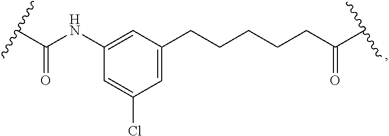 [SMILES: *C(=O)CCCCCc1cc(Cl)cc(NC(*)=O)c1] | (664) |
| 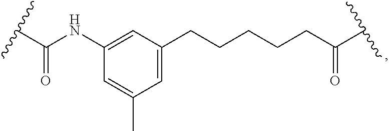 [SMILES: *C(=O)CCCCCc1cc(C)cc(NC(*)=O)c1] | (665) |
| 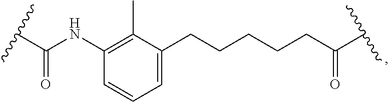 [SMILES: *C(=O)CCCCCc1cccc(NC(*)=O)c1C] | (666) |
| 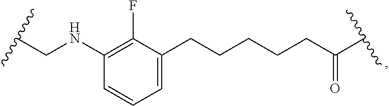 [SMILES: *CNc1cccc(CCCCCC(*)=O)c1F] | (667) |
| 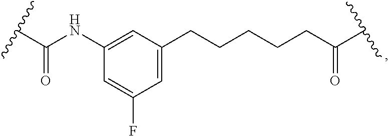 [SMILES: *C(=O)CCCCCc1cc(F)cc(NC(*)=O)c1] | (668) |
| 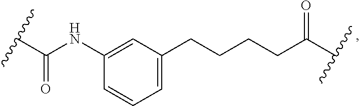 [SMILES: *C(=O)CCCCc1cccc(NC(*)=O)c1] | (669) |
| 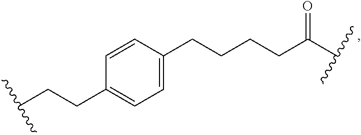 [SMILES: *CCc1ccc(CCCCC(*)=O)cc1] | (670) |
| 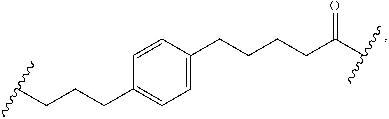 [SMILES: *CCCc1ccc(CCCCC(*)=O)cc1] | (671) |
| 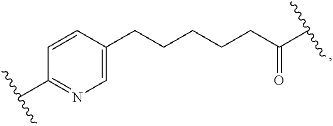 [SMILES: *C(=O)CCCCCc1ccc(*)nc1] | (672) |
| 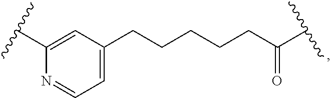 [SMILES: *C(=O)CCCCCc1ccnc(*)c1] | (673) |
| 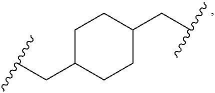 [SMILES: *CC1CCC(C*)CC1] | (674) |
| 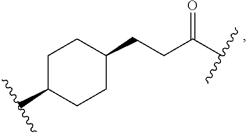 [SMILES: *C(=O)CC[C@H]1CC[C@@H](*)CC1] | (675) |
| 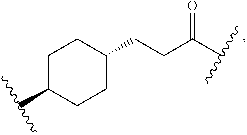 [SMILES: *C(=O)CC[C@H]1CC[C@H](*)CC1] | (676) |
| 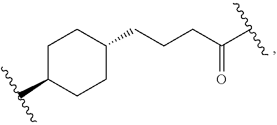 [SMILES: *C(=O)CCC[C@H]1CC[C@H](*)CC1] | (677) |
| 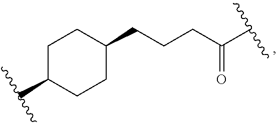 [SMILES: *C(=O)CCC[C@H]1CC[C@@H](*)CC1] | (678) |
| 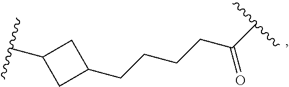 [SMILES: *C(=O)CCCCC1CC(*)C1] | (679) |
| 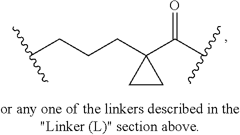 [SMILES: *CCCC1(C(*)=O)CC1] | (680) |

In some embodiments, the present invention provides a compound having MBM described and disclosed herein, LBM set forth in Table A above, and a linker set forth in Table B above, or a pharmaceutically acceptable salt thereof.

Exemplary compounds of the invention are set forth in Table 1, below.

TABLE 1
Exemplary Compounds

| # | Structure |
|---|---|
| I-1 | 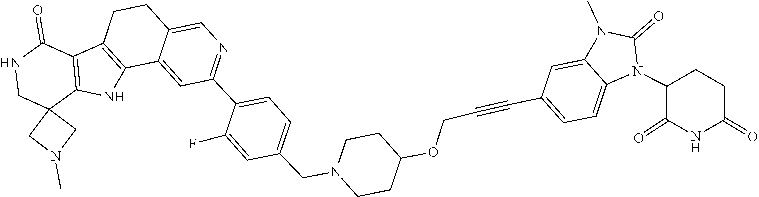 [SMILES: CN1CC2(CNC(=O)c3c2[nH]c2c3CCc3cnc(-c4ccc(CN5CCC(OCC#Cc6ccc7c(c6)n(C)c(=O)n7C6CCC(=O)NC6=O)CC5)cc4F)cc3-2)C1] |
| I-2 | 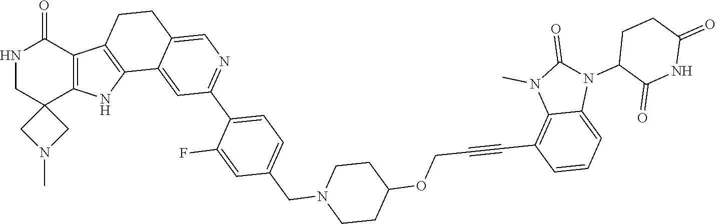 [SMILES: CN1CC2(CNC(=O)c3c2[nH]c2c3CCc3cnc(-c4ccc(CN5CCC(OCC#Cc6cccc7c6n(C)c(=O)n7C6CCC(=O)NC6=O)CC5)cc4F)cc3-2)C1] |
| I-3 | 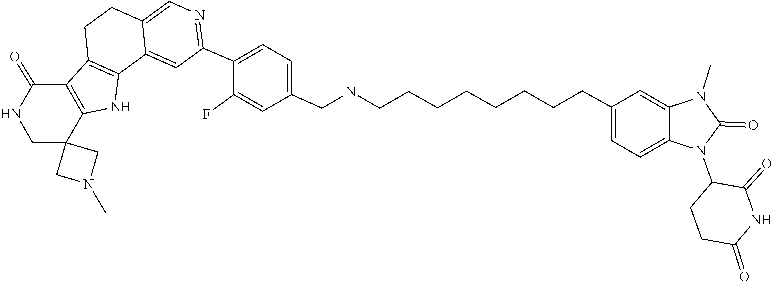 |
| I-4 | 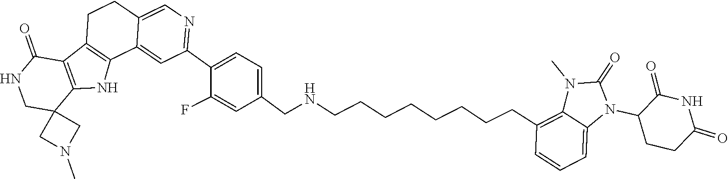 [SMILES: CN1CC2(CNC(=O)c3c2[nH]c2c3CCc3cnc(-c4ccc(CNCCCCCCCCc5cccc6c5n(C)c(=O)n6C5CCC(=O)NC5=O)cc4F)cc3-2)C1] |
| I-5 | 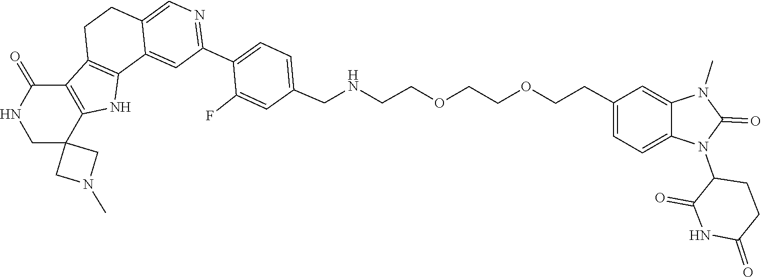 [SMILES: CN1CC2(CNC(=O)c3c2[nH]c2c3CCc3cnc(-c4ccc(CNCCOCCOCCc5ccc6c(c5)n(C)c(=O)n6C5CCC(=O)NC5=O)cc4F)cc3-2)C1] |
| I-6 | 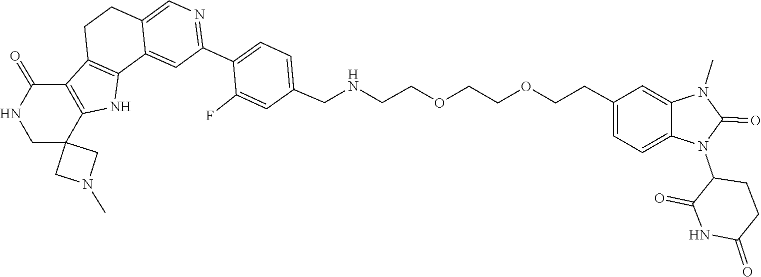 [SMILES: CN1CC2(CNC(=O)c3c2[nH]c2c3CCc3cnc(-c4ccc(CNCCOCCOCCc5ccc6c(c5)n(C)c(=O)n6C5CCC(=O)NC5=O)cc4F)cc3-2)C1] |
| I-7 | 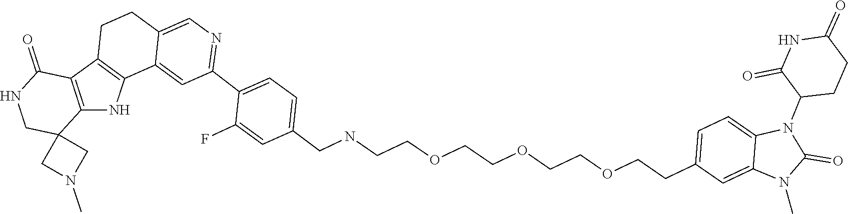 |
| I-8 | 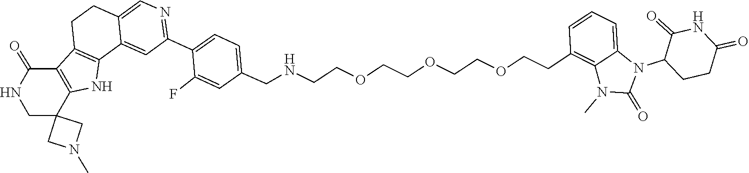 [SMILES: CN1CC2(CNC(=O)c3c2[nH]c2c3CCc3cnc(-c4ccc(CNCCOCCOCCOCCc5cccc6c5n(C)c(=O)n6C5CCC(=O)NC5=O)cc4F)cc3-2)C1] |
| I-9 | 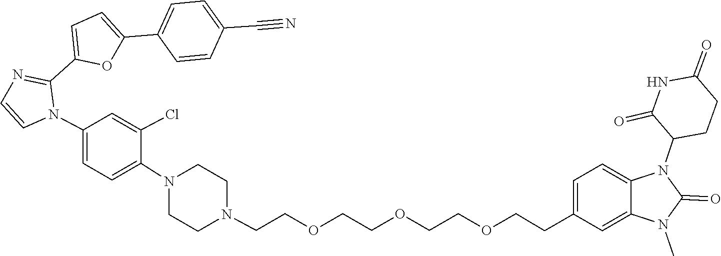 [SMILES: Cn1c(=O)n(C2CCC(=O)NC2=O)c2ccc(CCOCCOCCOCCN3CCN(c4ccc(-n5ccnc5-c5ccc(-c6ccc(C#N)cc6)o5)cc4Cl)CC3)cc21] |
| I-10 | 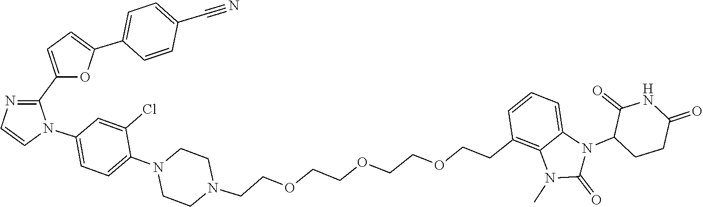 [SMILES: Cn1c(=O)n(C2CCC(=O)NC2=O)c2cccc(CCOCCOCCOCCN3CCN(c4ccc(-n5ccnc5-c5ccc(-c6ccc(C#N)cc6)o5)cc4Cl)CC3)c21] |
| I-11 | 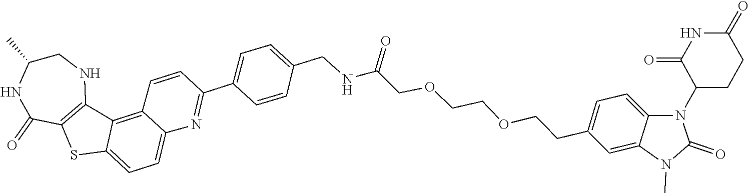 [SMILES: C[C@@H]1CNc2c(sc3ccc4nc(-c5ccc(CNC(=O)COCCOCCc6ccc7c(c6)n(C)c(=O)n7C6CCC(=O)NC6=O)cc5)ccc4c23)C(=O)N1] |
| I-12 | 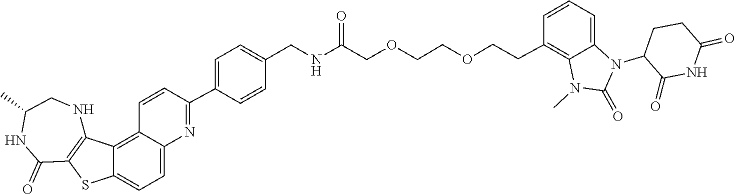 [SMILES: C[C@@H]1CNc2c(sc3ccc4nc(-c5ccc(CNC(=O)COCCOCCc6cccc7c6n(C)c(=O)n7C6CCC(=O)NC6=O)cc5)ccc4c23)C(=O)N1] |
| I-13 | 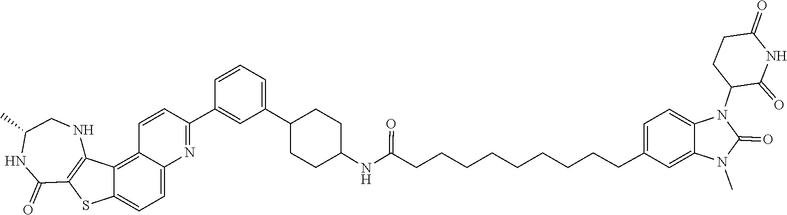 [SMILES: C[C@@H]1CNc2c(sc3ccc4nc(-c5cccc(C6CCC(NC(=O)CCCCCCCCCc7ccc8c(c7)n(C)c(=O)n8C7CCC(=O)NC7=O)CC6)c5)ccc4c23)C(=O)N1] |
| I-14 | 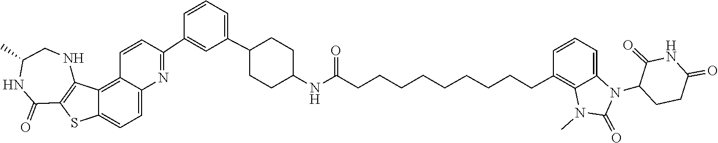 [SMILES: C[C@@H]1CNc2c(sc3ccc4nc(-c5cccc(C6CCC(NC(=O)CCCCCCCCCc7cccc8c7n(C)c(=O)n8C7CCC(=O)NC7=O)CC6)c5)ccc4c23)C(=O)N1] |
| I-15 | 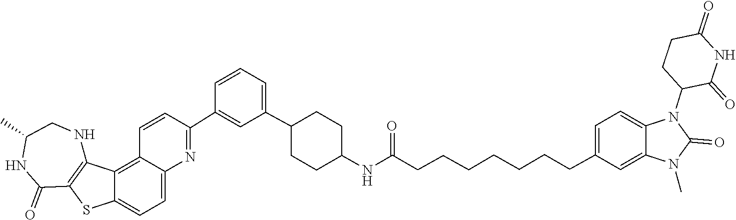 [SMILES: C[C@@H]1CNc2c(sc3ccc4nc(-c5cccc(C6CCC(NC(=O)CCCCCCCc7ccc8c(c7)n(C)c(=O)n8C7CCC(=O)NC7=O)CC6)c5)ccc4c23)C(=O)N1] |
| I-16 | 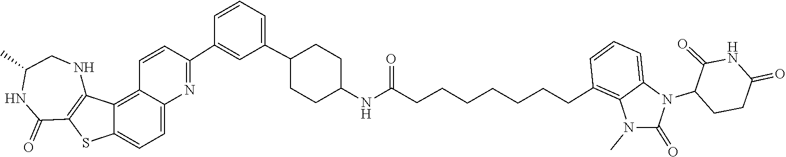 [SMILES: C[C@@H]1CNc2c(sc3ccc4nc(-c5cccc(C6CCC(NC(=O)CCCCCCCc7cccc8c7n(C)c(=O)n8C7CCC(=O)NC7=O)CC6)c5)ccc4c23)C(=O)N1] |
| I-17 | 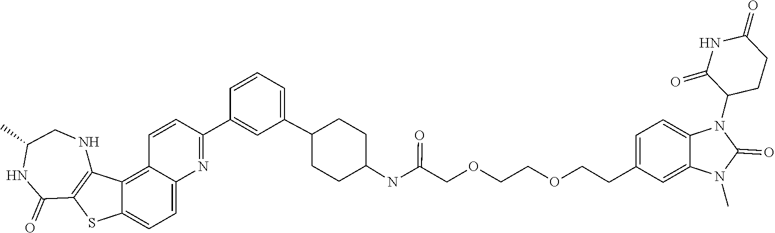 |
| I-18 | 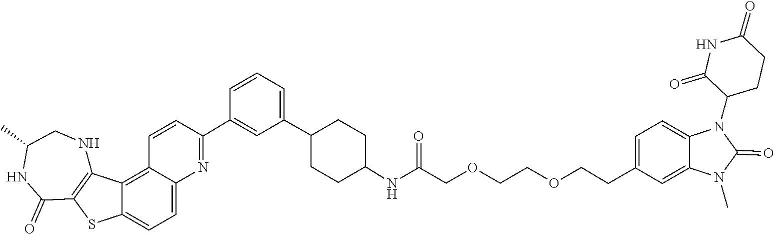 [SMILES: C[C@@H]1CNc2c(sc3ccc4nc(-c5cccc(C6CCC(NC(=O)COCCOCCc7ccc8c(c7)n(C)c(=O)n8C7CCC(=O)NC7=O)CC6)c5)ccc4c23)C(=O)N1] |
| I-19 | 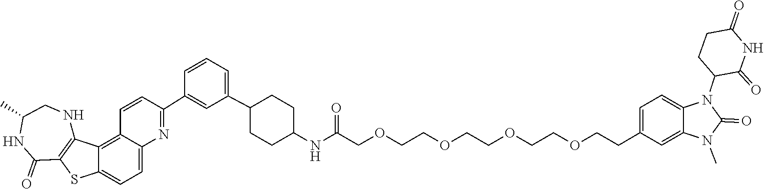 [SMILES: C[C@@H]1CNc2c(sc3ccc4nc(-c5cccc(C6CCC(NC(=O)COCCOCCOCCOCCc7ccc8c(c7)n(C)c(=O)n8C7CCC(=O)NC7=O)CC6)c5)ccc4c23)C(=O)N1] |
| I-20 | 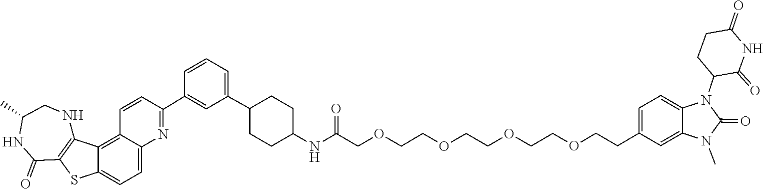 [SMILES: C[C@@H]1CNc2c(sc3ccc4nc(-c5cccc(C6CCC(NC(=O)COCCOCCOCCOCCc7ccc8c(c7)n(C)c(=O)n8C7CCC(=O)NC7=O)CC6)c5)ccc4c23)C(=O)N1] |
| I-21 | 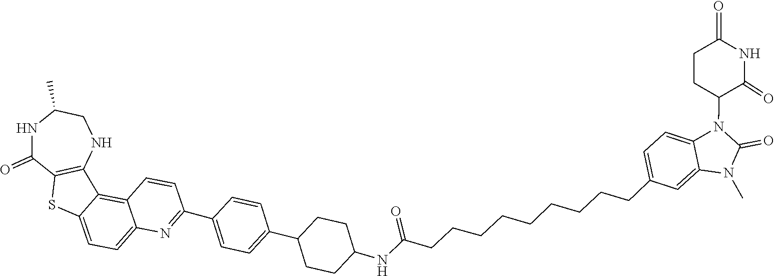 [SMILES: C[C@@H]1CNc2c(sc3ccc4nc(-c5ccc(C6CCC(NC(=O)CCCCCCCCCc7ccc8c(c7)n(C)c(=O)n8C7CCC(=O)NC7=O)CC6)cc5)ccc4c23)C(=O)N1] |
| I-22 | 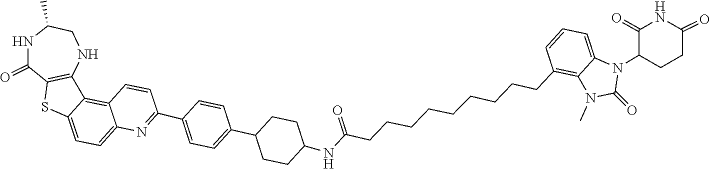 [SMILES: C[C@@H]1CNc2c(sc3ccc4nc(-c5ccc(C6CCC(NC(=O)CCCCCCCCCc7cccc8c7n(C)c(=O)n8C7CCC(=O)NC7=O)CC6)cc5)ccc4c23)C(=O)N1] |
| I-23 | 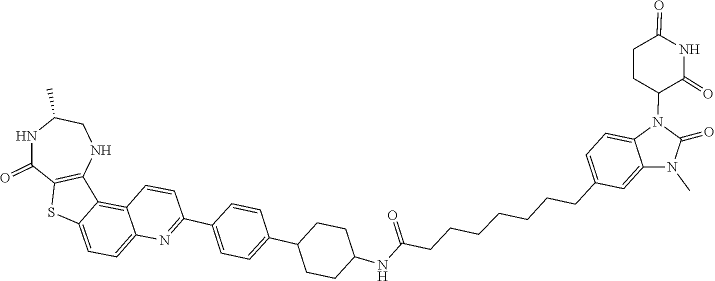 [SMILES: C[C@@H]1CNc2c(sc3ccc4nc(-c5ccc(C6CCC(NC(=O)CCCCCCCc7ccc8c(c7)n(C)c(=O)n8C7CCC(=O)NC7=O)CC6)cc5)ccc4c23)C(=O)N1] |
| I-24 | 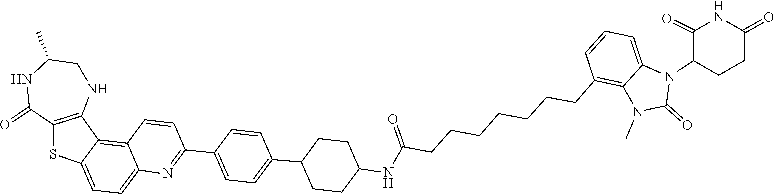 [SMILES: C[C@@H]1CNc2c(sc3ccc4nc(-c5ccc(C6CCC(NC(=O)CCCCCCCc7cccc8c7n(C)c(=O)n8C7CCC(=O)NC7=O)CC6)cc5)ccc4c23)C(=O)N1] |
| I-25 | 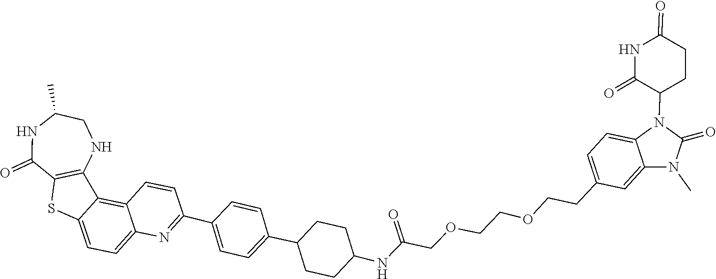 [SMILES: C[C@@H]1CNc2c(sc3ccc4nc(-c5ccc(C6CCC(NC(=O)COCCOCCc7ccc8c(c7)n(C)c(=O)n8C7CCC(=O)NC7=O)CC6)cc5)ccc4c23)C(=O)N1] |
| I-26 | 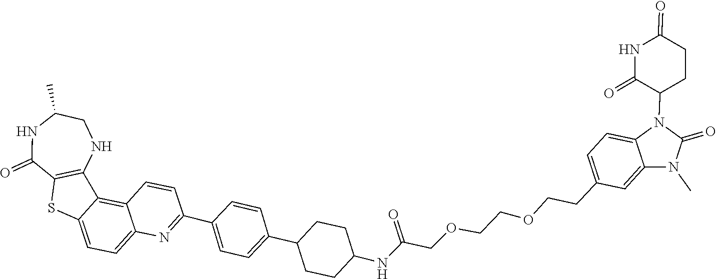 [SMILES: C[C@@H]1CNc2c(sc3ccc4nc(-c5ccc(C6CCC(NC(=O)COCCOCCc7ccc8c(c7)n(C)c(=O)n8C7CCC(=O)NC7=O)CC6)cc5)ccc4c23)C(=O)N1] |
| I-27 | 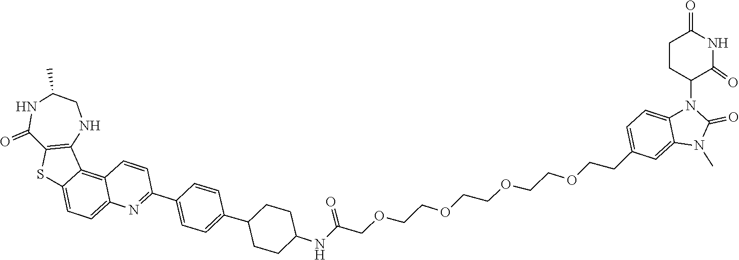 [SMILES: C[C@@H]1CNc2c(sc3ccc4nc(-c5ccc(C6CCC(NC(=O)COCCOCCOCCOCCc7ccc8c(c7)n(C)c(=O)n8C7CCC(=O)NC7=O)CC6)cc5)ccc4c23)C(=O)N1] |
| I-28 | 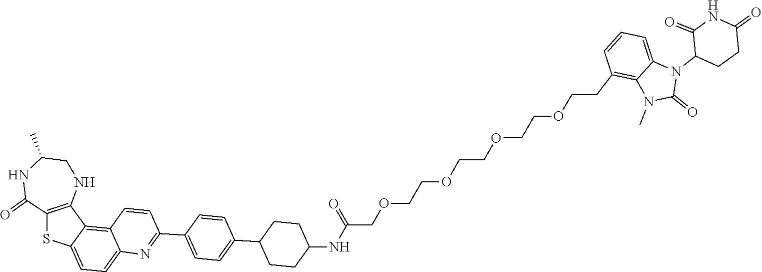 [SMILES: C[C@@H]1CNc2c(sc3ccc4nc(-c5ccc(C6CCC(NC(=O)COCCOCCOCCOCCc7cccc8c7n(C)c(=O)n8C7CCC(=O)NC7=O)CC6)cc5)ccc4c23)C(=O)N1] |
| I-29 | 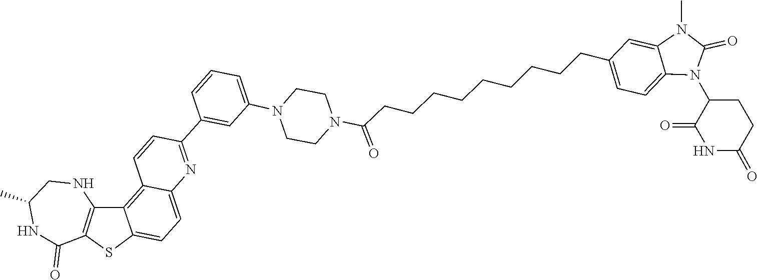 [SMILES: C[C@@H]1CNc2c(sc3ccc4nc(-c5cccc(N6CCN(C(=O)CCCCCCCCCc7ccc8c(c7)n(C)c(=O)n8C7CCC(=O)NC7=O)CC6)c5)ccc4c23)C(=O)N1] |
| I-30 | 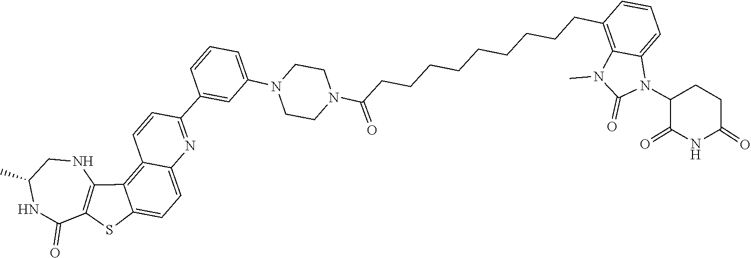 [SMILES: C[C@@H]1CNc2c(sc3ccc4nc(-c5cccc(N6CCN(C(=O)CCCCCCCCCc7cccc8c7n(C)c(=O)n8C7CCC(=O)NC7=O)CC6)c5)ccc4c23)C(=O)N1] |
| I-31 | 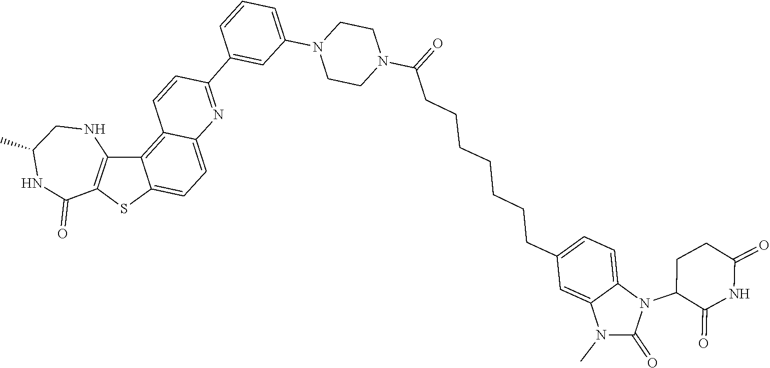 [SMILES: C[C@@H]1CNc2c(sc3ccc4nc(-c5cccc(N6CCN(C(=O)CCCCCCCc7ccc8c(c7)n(C)c(=O)n8C7CCC(=O)NC7=O)CC6)c5)ccc4c23)C(=O)N1] |
| I-32 | 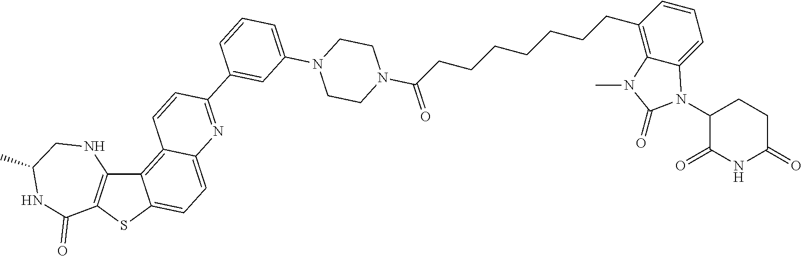 [SMILES: C[C@@H]1CNc2c(sc3ccc4nc(-c5cccc(N6CCN(C(=O)CCCCCCCc7cccc8c7n(C)c(=O)n8C7CCC(=O)NC7=O)CC6)c5)ccc4c23)C(=O)N1] |
| I-33 | 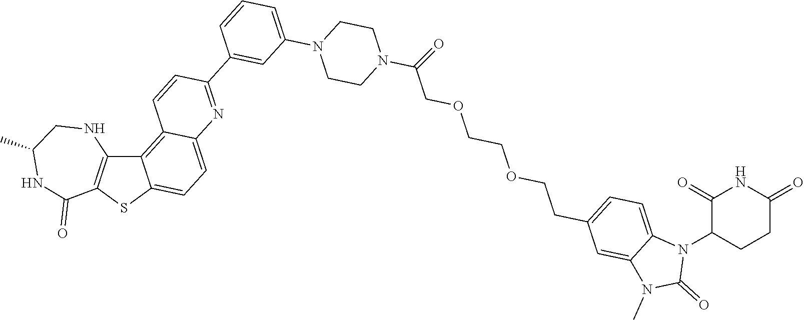 [SMILES: C[C@@H]1CNc2c(sc3ccc4nc(-c5cccc(N6CCN(C(=O)COCCOCCc7ccc8c(c7)n(C)c(=O)n8C7CCC(=O)NC7=O)CC6)c5)ccc4c23)C(=O)N1] |
| I-34 | 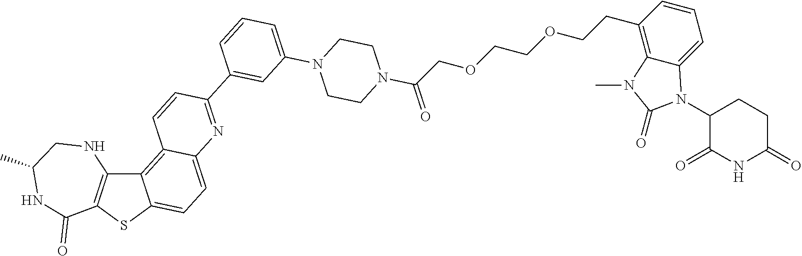 [SMILES: C[C@@H]1CNc2c(sc3ccc4nc(-c5cccc(N6CCN(C(=O)COCCOCCc7cccc8c7n(C)c(=O)n8C7CCC(=O)NC7=O)CC6)c5)ccc4c23)C(=O)N1] |
| I-36 | 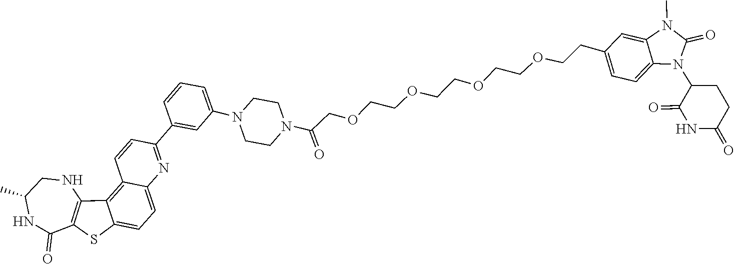 [SMILES: C[C@@H]1CNc2c(sc3ccc4nc(-c5cccc(N6CCN(C(=O)COCCOCCOCCOCCc7ccc8c(c7)n(C)c(=O)n8C7CCC(=O)NC7=O)CC6)c5)ccc4c23)C(=O)N1] |
| I-37 | 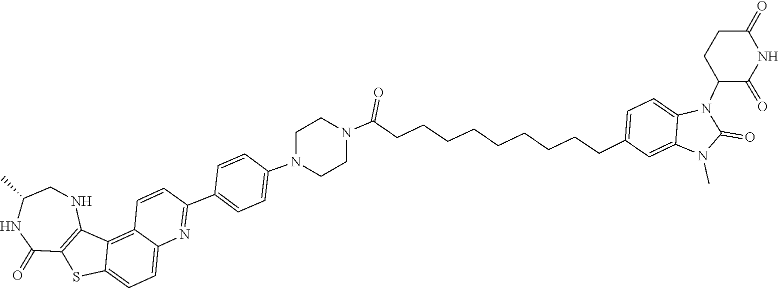 [SMILES: C[C@@H]1CNc2c(sc3ccc4nc(-c5ccc(N6CCN(C(=O)CCCCCCCCCc7ccc8c(c7)n(C)c(=O)n8C7CCC(=O)NC7=O)CC6)cc5)ccc4c23)C(=O)N1] |
| I-38 | 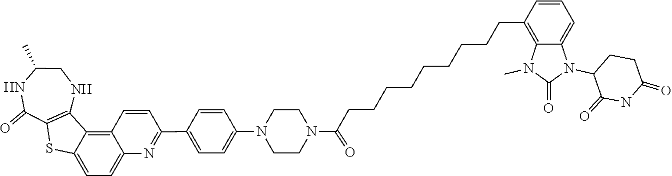 |
| I-39 | 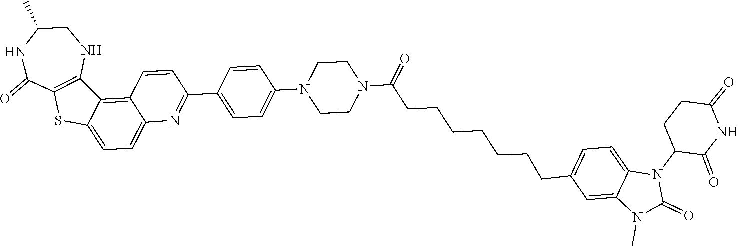 [SMILES: C[C@@H]1CNc2c(sc3ccc4nc(-c5ccc(N6CCN(C(=O)CCCCCCCc7ccc8c(c7)n(C)c(=O)n8C7CCC(=O)NC7=O)CC6)cc5)ccc4c23)C(=O)N1] |
| I-40 | 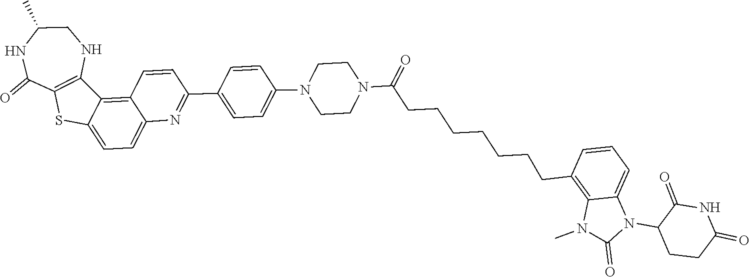 [SMILES: C[C@@H]1CNc2c(sc3ccc4nc(-c5ccc(N6CCN(C(=O)CCCCCCCc7cccc8c7n(C)c(=O)n8C7CCC(=O)NC7=O)CC6)cc5)ccc4c23)C(=O)N1] |
| I-41 | 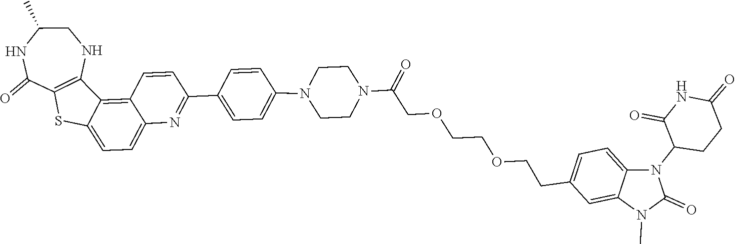 [SMILES: C[C@@H]1CNc2c(sc3ccc4nc(-c5ccc(N6CCN(C(=O)COCCOCCc7ccc8c(c7)n(C)c(=O)n8C7CCC(=O)NC7=O)CC6)cc5)ccc4c23)C(=O)N1] |
| I-42 | 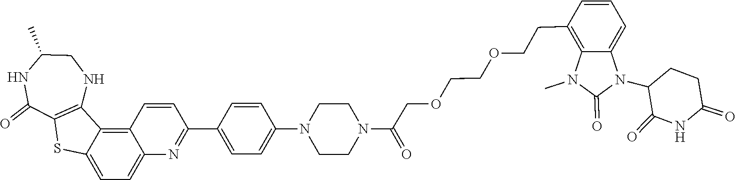 [SMILES: C[C@@H]1CNc2c(sc3ccc4nc(-c5ccc(N6CCN(C(=O)COCCOCCc7cccc8c7n(C)c(=O)n8C7CCC(=O)NC7=O)CC6)cc5)ccc4c23)C(=O)N1] |
| I-43 | 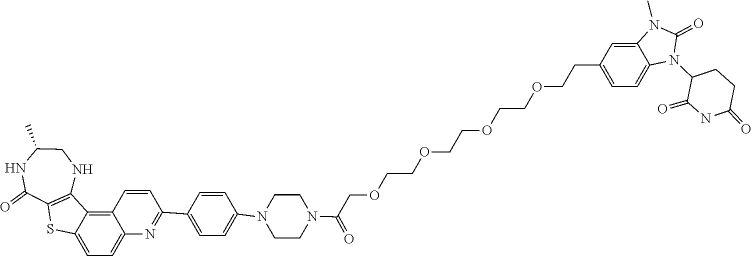 |
| I-44 | 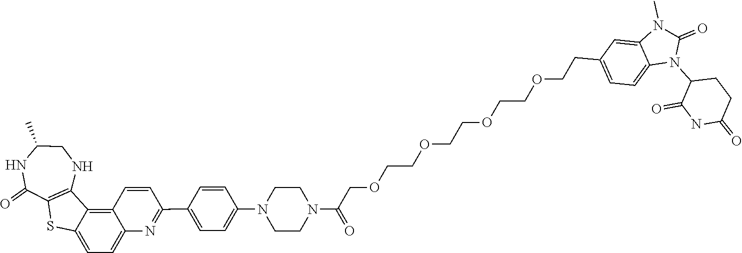 |
| I-45 | 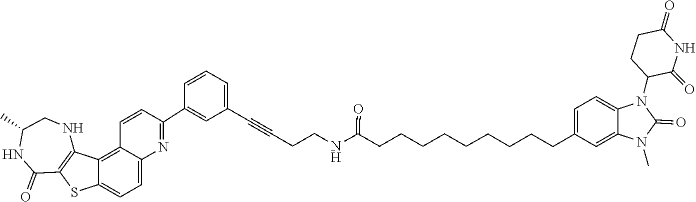 [SMILES: C[C@@H]1CNc2c(sc3ccc4nc(-c5cccc(C#CCCNC(=O)CCCCCCCCCc6ccc7c(c6)n(C)c(=O)n7C6CCC(=O)NC6=O)c5)ccc4c23)C(=O)N1] |
| I-46 | 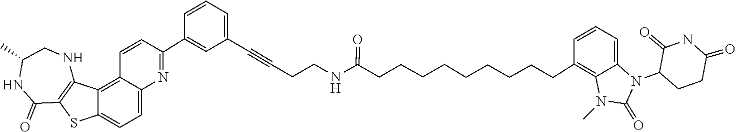 |
| I-47 | 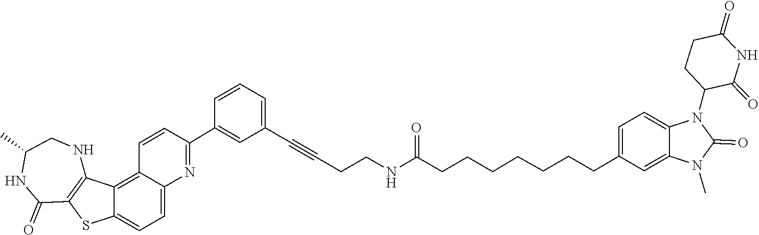 [SMILES: C[C@@H]1CNc2c(sc3ccc4nc(-c5cccc(C#CCCNC(=O)CCCCCCCc6ccc7c(c6)n(C)c(=O)n7C6CCC(=O)NC6=O)c5)ccc4c23)C(=O)N1] |
| I-48 | 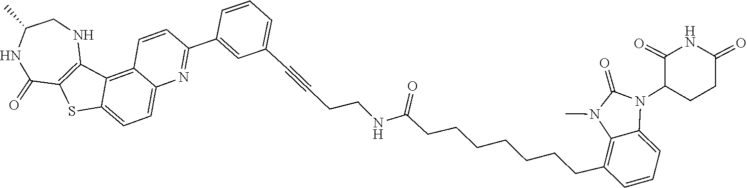 [SMILES: C[C@@H]1CNc2c(sc3ccc4nc(-c5cccc(C#CCCNC(=O)CCCCCCCc6cccc7c6n(C)c(=O)n7C6CCC(=O)NC6=O)c5)ccc4c23)C(=O)N1] |
| I-49 | 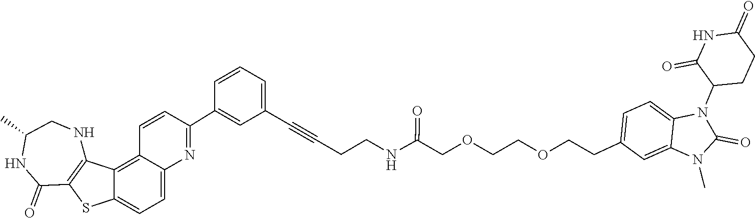 [SMILES: C[C@@H]1CNc2c(sc3ccc4nc(-c5cccc(C#CCCNC(=O)COCCOCCc6ccc7c(c6)n(C)c(=O)n7C6CCC(=O)NC6=O)c5)ccc4c23)C(=O)N1] |
| I-50 | 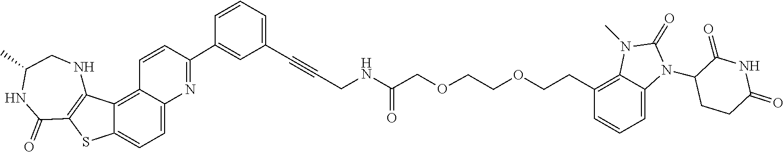 [SMILES: C[C@@H]1CNc2c(sc3ccc4nc(-c5cccc(C#CCNC(=O)COCCOCCc6cccc7c6n(C)c(=O)n7C6CCC(=O)NC6=O)c5)ccc4c23)C(=O)N1] |
| I-51 | 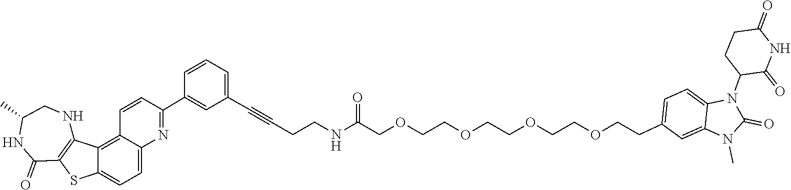 [SMILES: C[C@@H]1CNc2c(sc3ccc4nc(-c5cccc(C#CCCNC(=O)COCCOCCOCCOCCc6ccc7c(c6)n(C)c(=O)n7C6CCC(=O)NC6=O)c5)ccc4c23)C(=O)N1] |
| I-52 | 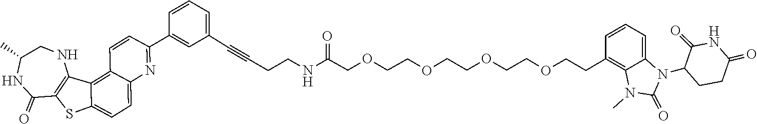 [SMILES: C[C@@H]1CNc2c(sc3ccc4nc(-c5cccc(C#CCCNC(=O)COCCOCCOCCOCCc6cccc7c6n(C)c(=O)n7C6CCC(=O)NC6=O)c5)ccc4c23)C(=O)N1] |
| I-53 | 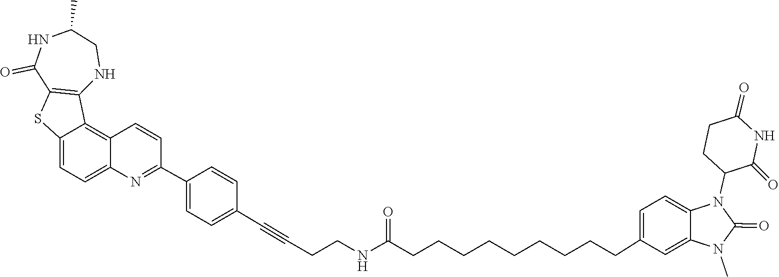 [SMILES: C[C@@H]1CNc2c(sc3ccc4nc(-c5ccc(C#CCCNC(=O)CCCCCCCCCc6ccc7c(c6)n(C)c(=O)n7C6CCC(=O)NC6=O)cc5)ccc4c23)C(=O)N1] |
| I-54 | 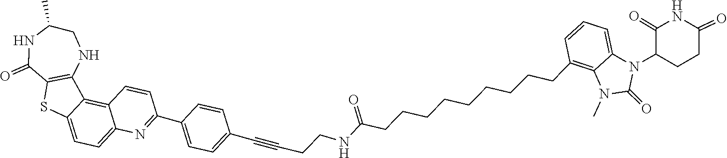 [SMILES: C[C@@H]1CNc2c(sc3ccc4nc(-c5ccc(C#CCCNC(=O)CCCCCCCCCc6cccc7c6n(C)c(=O)n7C6CCC(=O)NC6=O)cc5)ccc4c23)C(=O)N1] |
| I-55 | 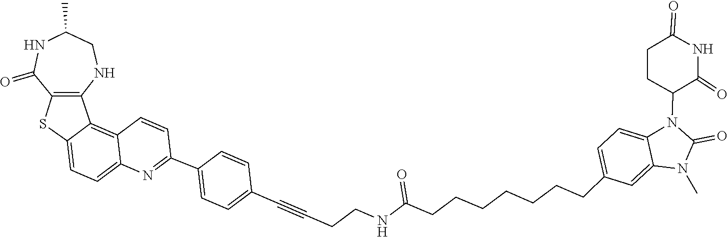 [SMILES: C[C@@H]1CNc2c(sc3ccc4nc(-c5ccc(C#CCCNC(=O)CCCCCCCc6ccc7c(c6)n(C)c(=O)n7C6CCC(=O)NC6=O)cc5)ccc4c23)C(=O)N1] |
| I-56 | 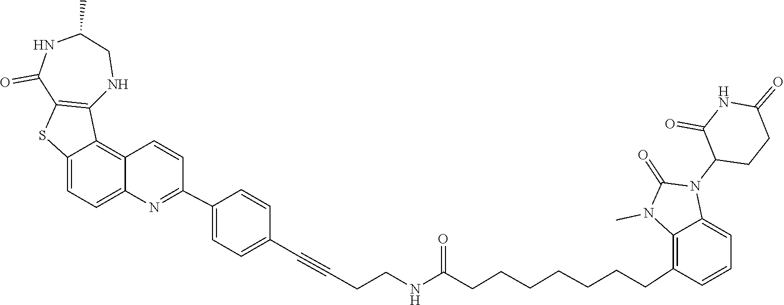 [SMILES: C[C@@H]1CNc2c(sc3ccc4nc(-c5ccc(C#CCCNC(=O)CCCCCCCc6cccc7c6n(C)c(=O)n7C6CCC(=O)NC6=O)cc5)ccc4c23)C(=O)N1] |
| I-57 | 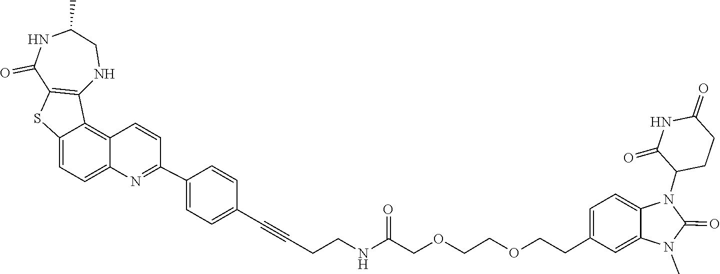 [SMILES: C[C@@H]1CNc2c(sc3ccc4nc(-c5ccc(C#CCCNC(=O)COCCOCCc6ccc7c(c6)n(C)c(=O)n7C6CCC(=O)NC6=O)cc5)ccc4c23)C(=O)N1] |
| I-58 | 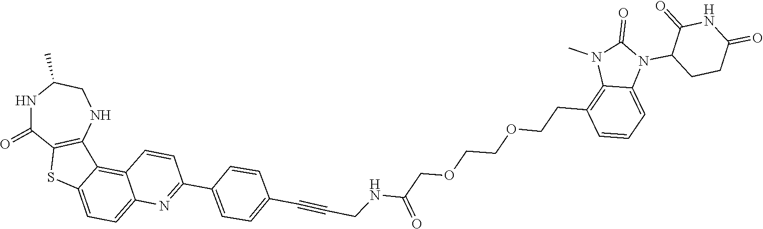 [SMILES: C[C@@H]1CNc2c(sc3ccc4nc(-c5ccc(C#CCNC(=O)COCCOCCc6cccc7c6n(C)c(=O)n7C6CCC(=O)NC6=O)cc5)ccc4c23)C(=O)N1] |
| I-59 | 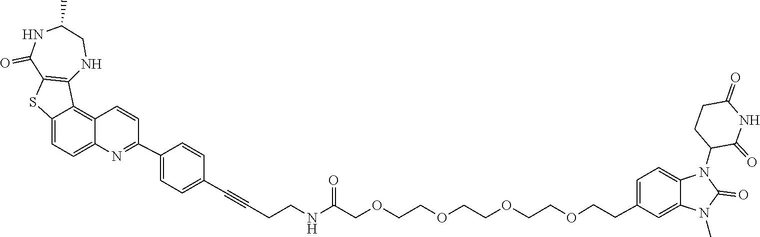 [SMILES: C[C@@H]1CNc2c(sc3ccc4nc(-c5ccc(C#CCCNC(=O)COCCOCCOCCOCCc6ccc7c(c6)n(C)c(=O)n7C6CCC(=O)NC6=O)cc5)ccc4c23)C(=O)N1] |
| I-60 | 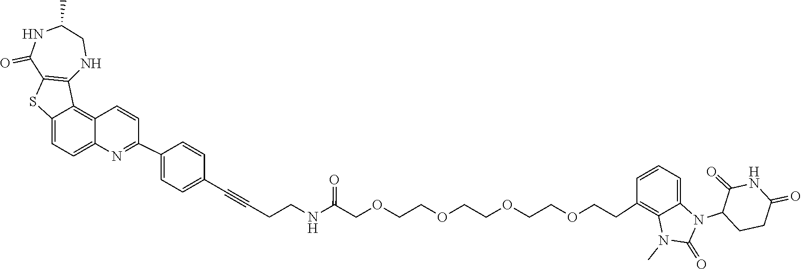 [SMILES: C[C@@H]1CNc2c(sc3ccc4nc(-c5ccc(C#CCCNC(=O)COCCOCCOCCOCCc6cccc7c6n(C)c(=O)n7C6CCC(=O)NC6=O)cc5)ccc4c23)C(=O)N1] |
| I-61 | 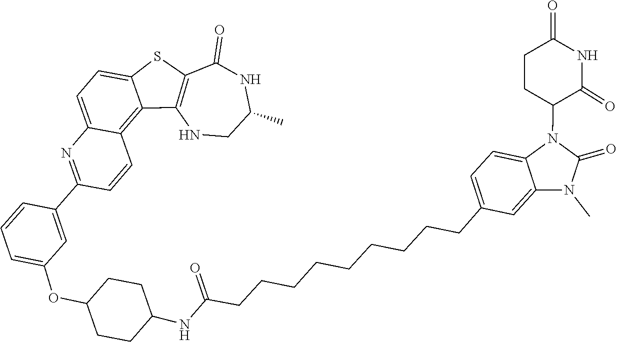 [SMILES: C[C@@H]1CNc2c(sc3ccc4nc(-c5cccc(OC6CCC(NC(=O)CCCCCCCCCc7ccc8c(c7)n(C)c(=O)n8C7CCC(=O)NC7=O)CC6)c5)ccc4c23)C(=O)N1] |
| I-62 | 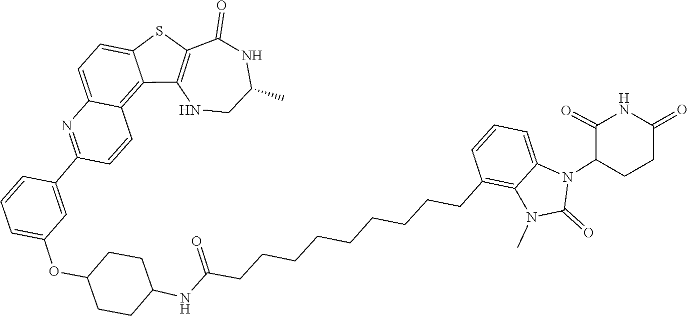 [SMILES: C[C@@H]1CNc2c(sc3ccc4nc(-c5cccc(OC6CCC(NC(=O)CCCCCCCCCc7cccc8c7n(C)c(=O)n8C7CCC(=O)NC7=O)CC6)c5)ccc4c23)C(=O)N1] |
| I-63 | 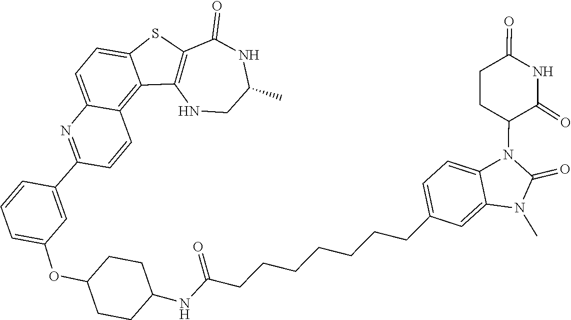 [SMILES: C[C@@H]1CNc2c(sc3ccc4nc(-c5cccc(OC6CCC(NC(=O)CCCCCCCc7ccc8c(c7)n(C)c(=O)n8C7CCC(=O)NC7=O)CC6)c5)ccc4c23)C(=O)N1] |
| I-64 | 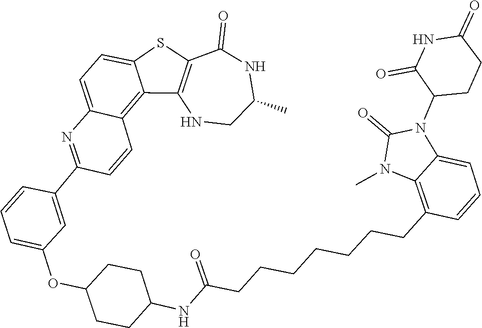 [SMILES: C[C@@H]1CNc2c(sc3ccc4nc(-c5cccc(OC6CCC(NC(=O)CCCCCCCc7cccc8c7n(C)c(=O)n8C7CCC(=O)NC7=O)CC6)c5)ccc4c23)C(=O)N1] |
| I-65 | 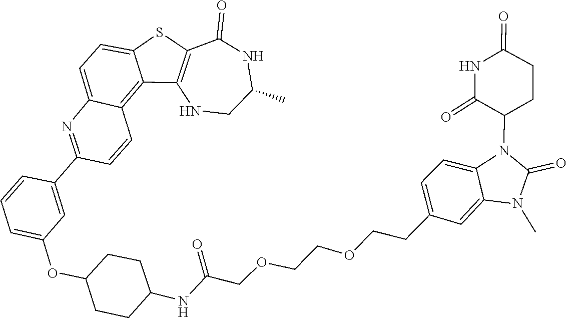 [SMILES: C[C@@H]1CNc2c(sc3ccc4nc(-c5cccc(OC6CCC(NC(=O)COCCOCCc7ccc8c(c7)n(C)c(=O)n8C7CCC(=O)NC7=O)CC6)c5)ccc4c23)C(=O)N1] |
| I-66 | 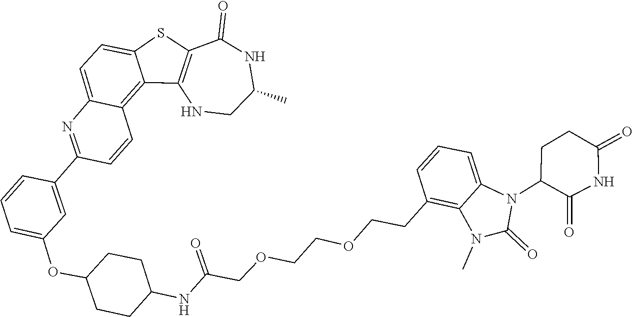 [SMILES: C[C@@H]1CNc2c(sc3ccc4nc(-c5cccc(OC6CCC(NC(=O)COCCOCCc7cccc8c7n(C)c(=O)n8C7CCC(=O)NC7=O)CC6)c5)ccc4c23)C(=O)N1] |
| I-67 | 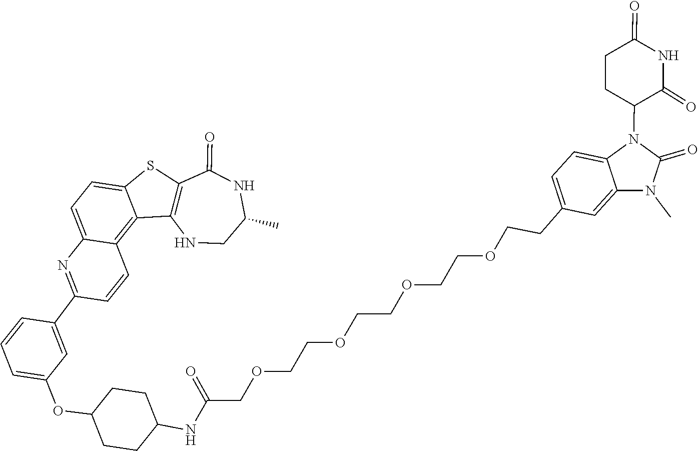 [SMILES: C[C@@H]1CNc2c(sc3ccc4nc(-c5cccc(OC6CCC(NC(=O)COCCOCCOCCOCCc7ccc8c(c7)n(C)c(=O)n8C7CCC(=O)NC7=O)CC6)c5)ccc4c23)C(=O)N1] |
| I-68 | 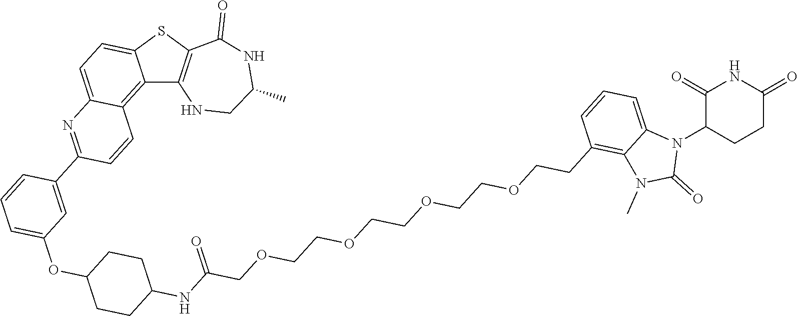 [SMILES: C[C@@H]1CNc2c(sc3ccc4nc(-c5cccc(OC6CCC(NC(=O)COCCOCCOCCOCCc7cccc8c7n(C)c(=O)n8C7CCC(=O)NC7=O)CC6)c5)ccc4c23)C(=O)N1] |
| I-69 | 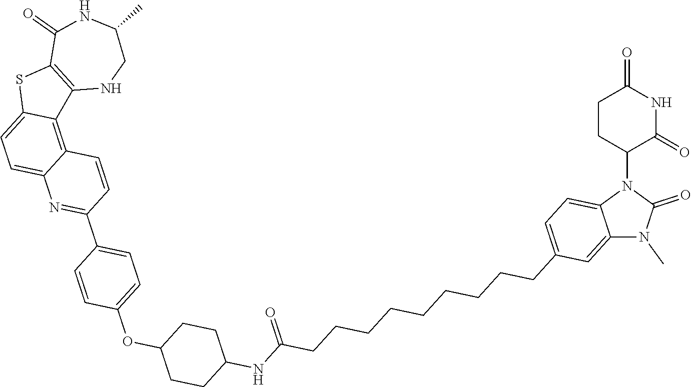 [SMILES: C[C@@H]1CNc2c(sc3ccc4nc(-c5ccc(OC6CCC(NC(=O)CCCCCCCCCc7ccc8c(c7)n(C)c(=O)n8C7CCC(=O)NC7=O)CC6)cc5)ccc4c23)C(=O)N1] |
| I-70 | 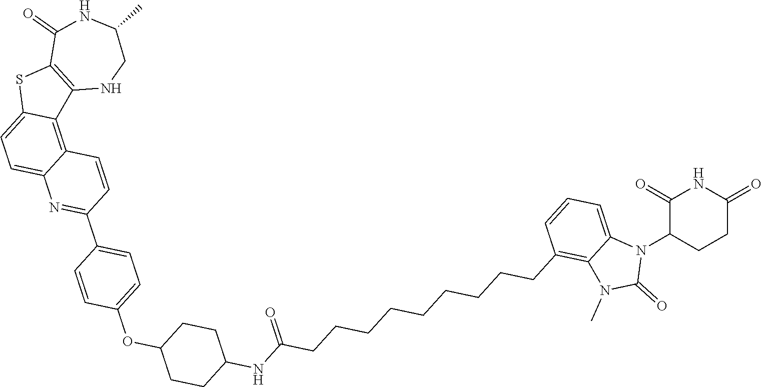 [SMILES: C[C@@H]1CNc2c(sc3ccc4nc(-c5ccc(OC6CCC(NC(=O)CCCCCCCCCc7cccc8c7n(C)c(=O)n8C7CCC(=O)NC7=O)CC6)cc5)ccc4c23)C(=O)N1] |
| I-71 | 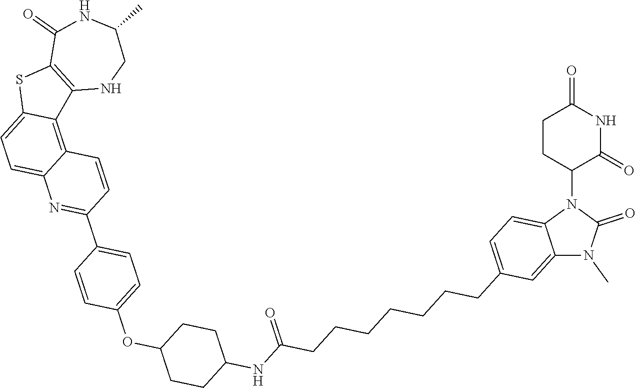 [SMILES: C[C@@H]1CNc2c(sc3ccc4nc(-c5ccc(OC6CCC(NC(=O)CCCCCCCc7ccc8c(c7)n(C)c(=O)n8C7CCC(=O)NC7=O)CC6)cc5)ccc4c23)C(=O)N1] |
| I-72 | 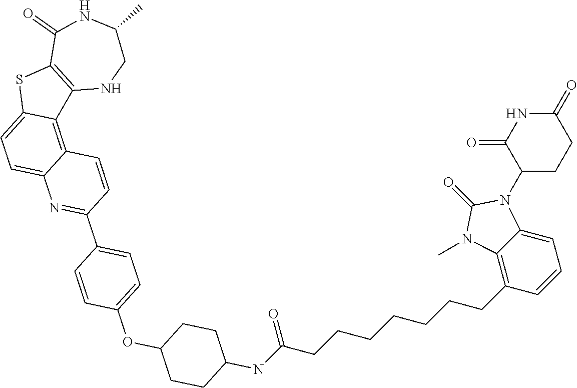 |
| I-73 | 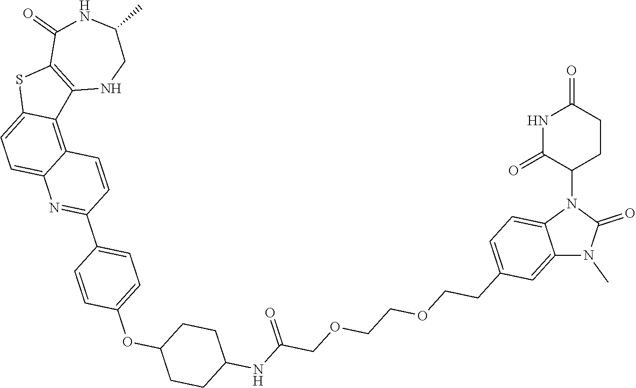 [SMILES: C[C@@H]1CNc2c(sc3ccc4nc(-c5ccc(OC6CCC(NC(=O)COCCOCCc7ccc8c(c7)n(C)c(=O)n8C7CCC(=O)NC7=O)CC6)cc5)ccc4c23)C(=O)N1] |
| I-74 | 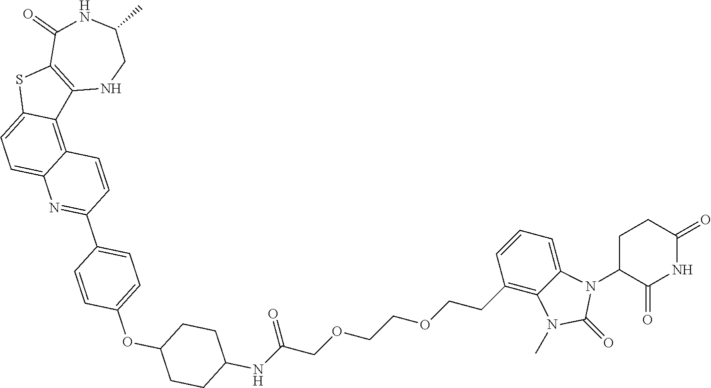 [SMILES: C[C@@H]1CNc2c(sc3ccc4nc(-c5ccc(OC6CCC(NC(=O)COCCOCCc7cccc8c7n(C)c(=O)n8C7CCC(=O)NC7=O)CC6)cc5)ccc4c23)C(=O)N1] |
| I-75 | 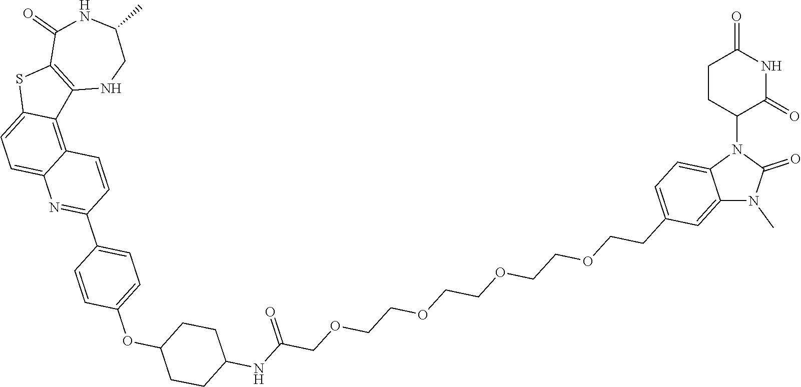 [SMILES: C[C@@H]1CNc2c(sc3ccc4nc(-c5ccc(OC6CCC(NC(=O)COCCOCCOCCOCCc7ccc8c(c7)n(C)c(=O)n8C7CCC(=O)NC7=O)CC6)cc5)ccc4c23)C(=O)N1] |
| I-76 | 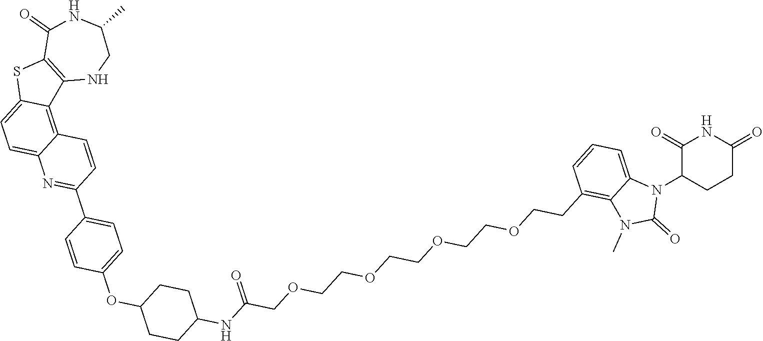 [SMILES: C[C@@H]1CNc2c(sc3ccc4nc(-c5ccc(OC6CCC(NC(=O)COCCOCCOCCOCCc7cccc8c7n(C)c(=O)n8C7CCC(=O)NC7=O)CC6)cc5)ccc4c23)C(=O)N1] |
| I-77 | 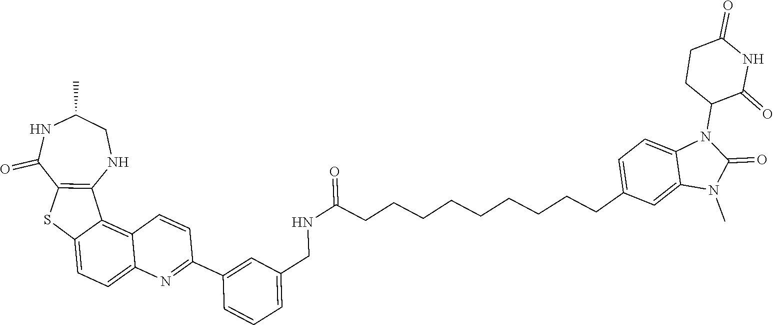 [SMILES: C[C@@H]1CNc2c(sc3ccc4nc(-c5cccc(CNC(=O)CCCCCCCCCc6ccc7c(c6)n(C)c(=O)n7C6CCC(=O)NC6=O)c5)ccc4c23)C(=O)N1] |
| I-78 | 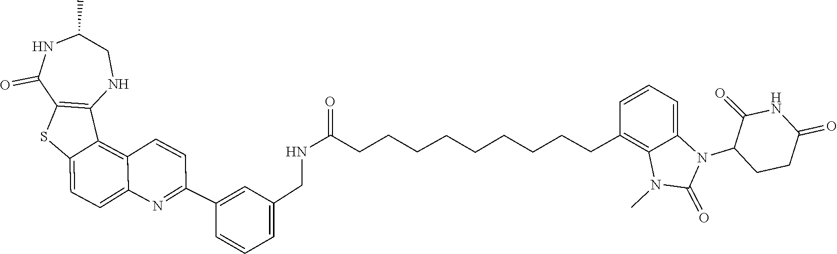 [SMILES: C[C@@H]1CNc2c(sc3ccc4nc(-c5cccc(CNC(=O)CCCCCCCCCc6cccc7c6n(C)c(=O)n7C6CCC(=O)NC6=O)c5)ccc4c23)C(=O)N1] |
| I-79 | 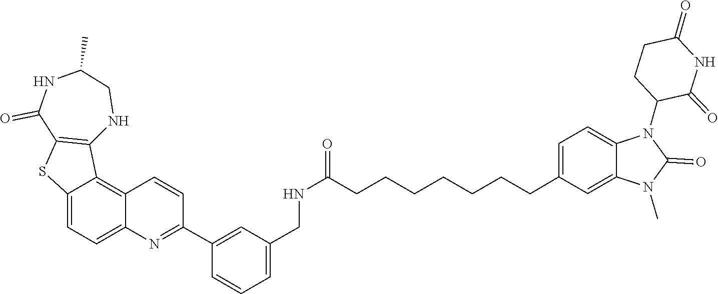 [SMILES: C[C@@H]1CNc2c(sc3ccc4nc(-c5cccc(CNC(=O)CCCCCCCc6ccc7c(c6)n(C)c(=O)n7C6CCC(=O)NC6=O)c5)ccc4c23)C(=O)N1] |
| I-80 | 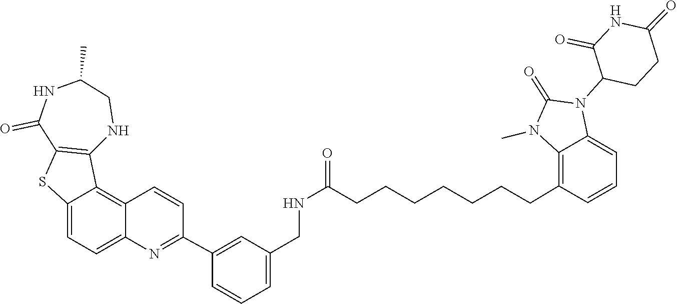 [SMILES: C[C@@H]1CNc2c(sc3ccc4nc(-c5cccc(CNC(=O)CCCCCCCc6cccc7c6n(C)c(=O)n7C6CCC(=O)NC6=O)c5)ccc4c23)C(=O)N1] |
| I-81 | 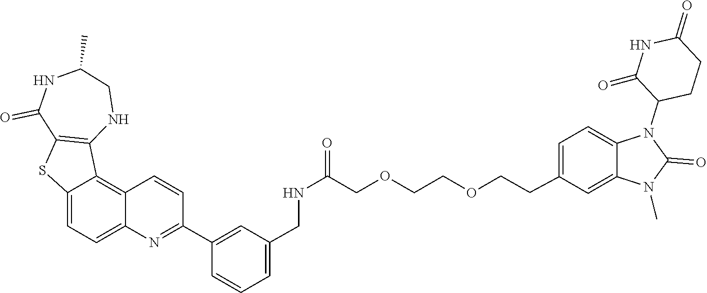 [SMILES: C[C@@H]1CNc2c(sc3ccc4nc(-c5cccc(CNC(=O)COCCOCCc6ccc7c(c6)n(C)c(=O)n7C6CCC(=O)NC6=O)c5)ccc4c23)C(=O)N1] |
| I-82 | 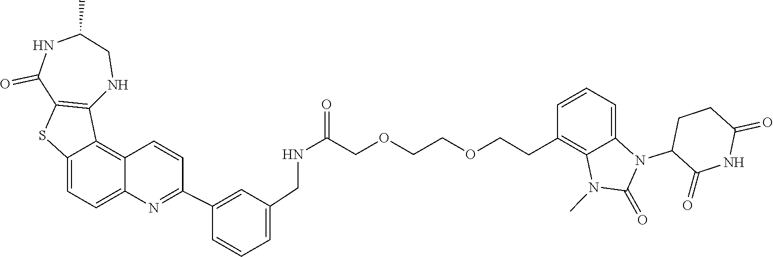 [SMILES: C[C@@H]1CNc2c(sc3ccc4nc(-c5cccc(CNC(=O)COCCOCCc6cccc7c6n(C)c(=O)n7C6CCC(=O)NC6=O)c5)ccc4c23)C(=O)N1] |
| I-83 | 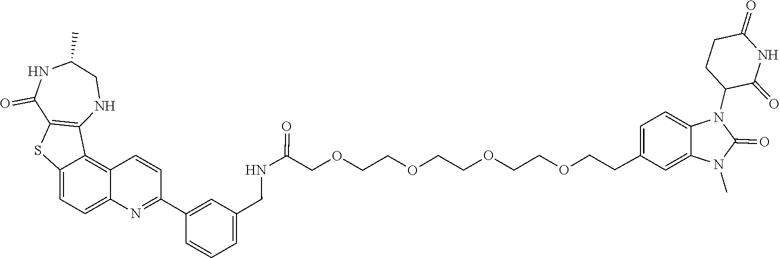 [SMILES: C[C@@H]1CNc2c(sc3ccc4nc(-c5cccc(CNC(=O)COCCOCCOCCOCCc6ccc7c(c6)n(C)c(=O)n7C6CCC(=O)NC6=O)c5)ccc4c23)C(=O)N1] |
| I-84 | 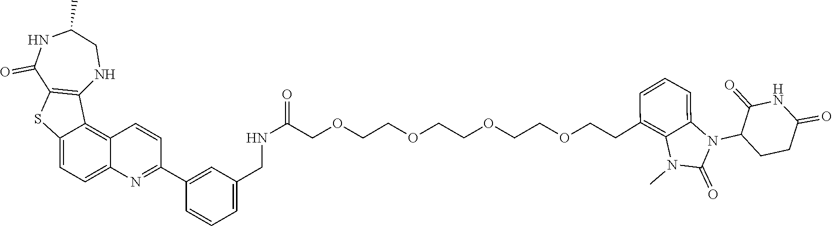 [SMILES: C[C@@H]1CNc2c(sc3ccc4nc(-c5cccc(CNC(=O)COCCOCCOCCOCCc6cccc7c6n(C)c(=O)n7C6CCC(=O)NC6=O)c5)ccc4c23)C(=O)N1] |
| I-85 | 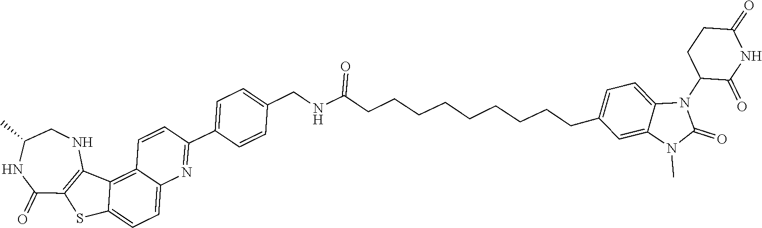 [SMILES: C[C@@H]1CNc2c(sc3ccc4nc(-c5ccc(CNC(=O)CCCCCCCCCc6ccc7c(c6)n(C)c(=O)n7C6CCC(=O)NC6=O)cc5)ccc4c23)C(=O)N1] |
| I-86 | 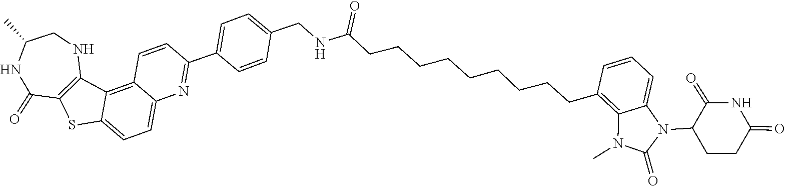 [SMILES: C[C@@H]1CNc2c(sc3ccc4nc(-c5ccc(CNC(=O)CCCCCCCCCc6cccc7c6n(C)c(=O)n7C6CCC(=O)NC6=O)cc5)ccc4c23)C(=O)N1] |
| I-87 | 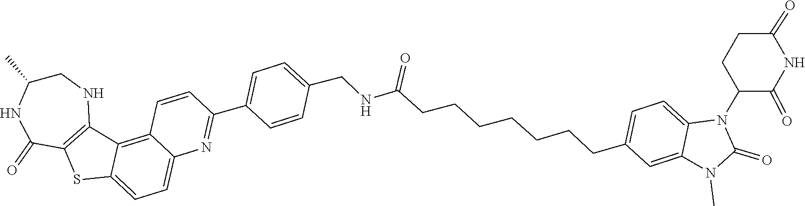 [SMILES: C[C@@H]1CNc2c(sc3ccc4nc(-c5ccc(CNC(=O)CCCCCCCc6ccc7c(c6)n(C)c(=O)n7C6CCC(=O)NC6=O)cc5)ccc4c23)C(=O)N1] |
| I-88 | 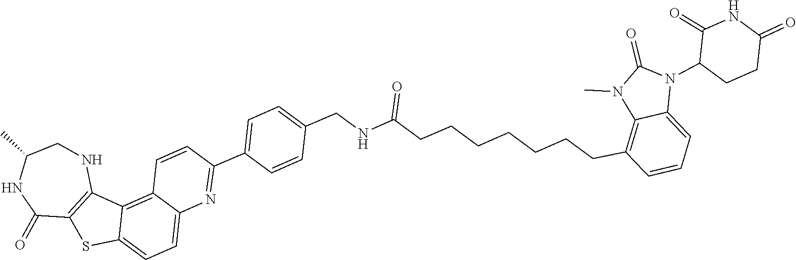 [SMILES: C[C@@H]1CNc2c(sc3ccc4nc(-c5ccc(CNC(=O)CCCCCCCc6cccc7c6n(C)c(=O)n7C6CCC(=O)NC6=O)cc5)ccc4c23)C(=O)N1] |
| I-91 | 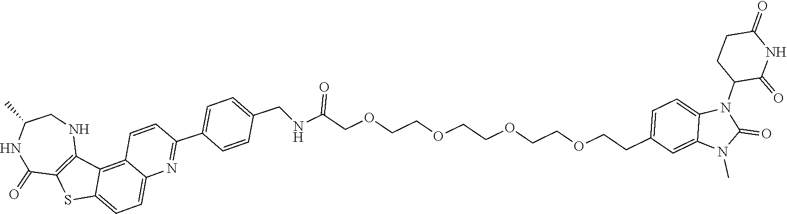 [SMILES: C[C@@H]1CNc2c(sc3ccc4nc(-c5ccc(CNC(=O)COCCOCCOCCOCCc6ccc7c(c6)n(C)c(=O)n7C6CCC(=O)NC6=O)cc5)ccc4c23)C(=O)N1] |
| I-92 | 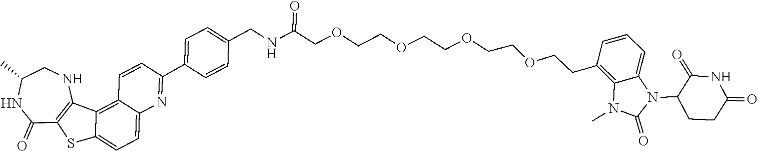 [SMILES: C[C@@H]1CNc2c(sc3ccc4nc(-c5ccc(CNC(=O)COCCOCCOCCOCCc6cccc7c6n(C)c(=O)n7C6CCC(=O)NC6=O)cc5)ccc4c23)C(=O)N1] |
| I-93 | 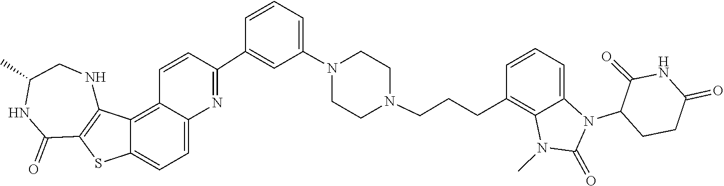 [SMILES: C[C@@H]1CNc2c(sc3ccc4nc(-c5cccc(N6CCN(CCCc7cccc8c7n(C)c(=O)n8C7CCC(=O)NC7=O)CC6)c5)ccc4c23)C(=O)N1] |
| I-94 | 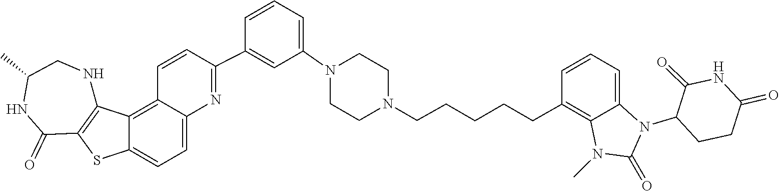 [SMILES: C[C@@H]1CNc2c(sc3ccc4nc(-c5cccc(N6CCN(CCCCCc7cccc8c7n(C)c(=O)n8C7CCC(=O)NC7=O)CC6)c5)ccc4c23)C(=O)N1] |
| I-95 | 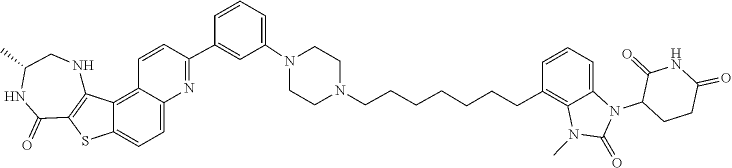 [SMILES: C[C@@H]1CNc2c(sc3ccc4nc(-c5cccc(N6CCN(CCCCCCCc7cccc8c7n(C)c(=O)n8C7CCC(=O)NC7=O)CC6)c5)ccc4c23)C(=O)N1] |
| I-96 | 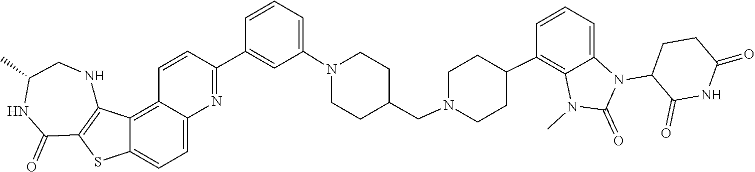 [SMILES: C[C@@H]1CNc2c(sc3ccc4nc(-c5cccc(N6CCC(CN7CCC(c8cccc9c8n(C)c(=O)n9C8CCC(=O)NC8=O)CC7)CC6)c5)ccc4c23)C(=O)N1] |
| I-97 | 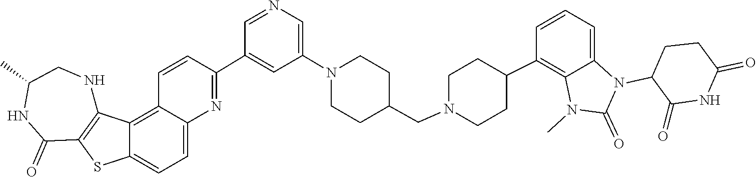 [SMILES: C[C@@H]1CNc2c(sc3ccc4nc(-c5cncc(N6CCC(CN7CCC(c8cccc9c8n(C)c(=O)n9C8CCC(=O)NC8=O)CC7)CC6)c5)ccc4c23)C(=O)N1] |
| I-98 | 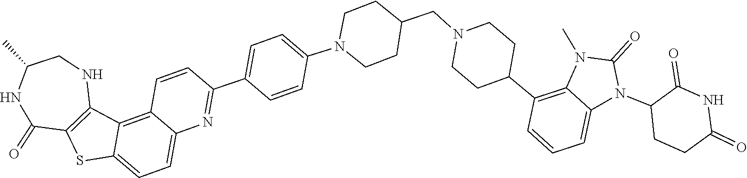 [SMILES: C[C@@H]1CNc2c(sc3ccc4nc(-c5ccc(N6CCC(CN7CCC(c8cccc9c8n(C)c(=O)n9C8CCC(=O)NC8=O)CC7)CC6)cc5)ccc4c23)C(=O)N1] |
| I-99 | 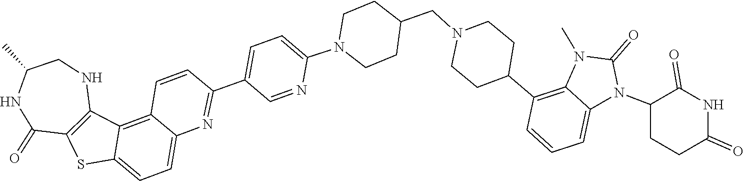 [SMILES: C[C@@H]1CNc2c(sc3ccc4nc(-c5ccc(N6CCC(CN7CCC(c8cccc9c8n(C)c(=O)n9C8CCC(=O)NC8=O)CC7)CC6)nc5)ccc4c23)C(=O)N1] |
| I-100 | 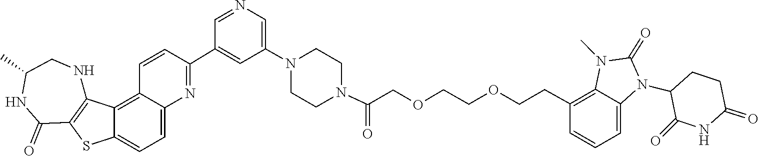 [SMILES: C[C@@H]1CNc2c(sc3ccc4nc(-c5cncc(N6CCN(C(=O)COCCOCCc7cccc8c7n(C)c(=O)n8C7CCC(=O)NC7=O)CC6)c5)ccc4c23)C(=O)N1] |
| I-101 | 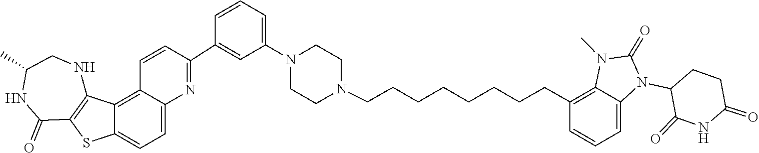 [SMILES: C[C@@H]1CNc2c(sc3ccc4nc(-c5cccc(N6CCN(CCCCCCCCc7cccc8c7n(C)c(=O)n8C7CCC(=O)NC7=O)CC6)c5)ccc4c23)C(=O)N1] |
| I-102 | 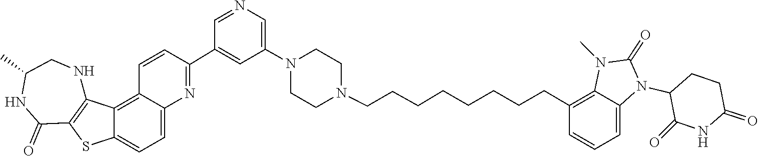 [SMILES: C[C@@H]1CNc2c(sc3ccc4nc(-c5cncc(N6CCN(CCCCCCCCc7cccc8c7n(C)c(=O)n8C7CCC(=O)NC7=O)CC6)c5)ccc4c23)C(=O)N1] |
| I-103 | 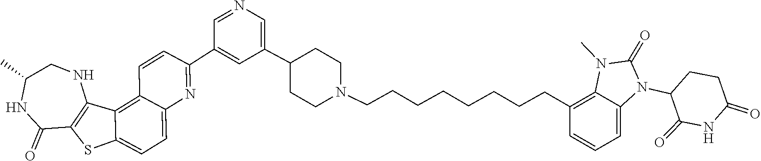 [SMILES: C[C@@H]1CNc2c(sc3ccc4nc(-c5cncc(C6CCN(CCCCCCCCc7cccc8c7n(C)c(=O)n8C7CCC(=O)NC7=O)CC6)c5)ccc4c23)C(=O)N1] |
| I-104 | 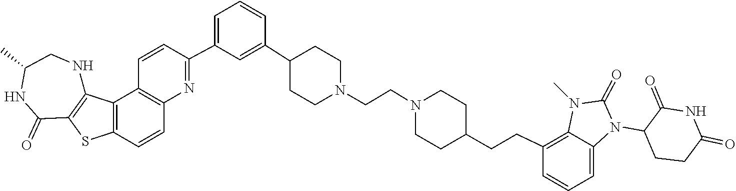 [SMILES: C[C@@H]1CNc2c(sc3ccc4nc(-c5cccc(C6CCN(CCN7CCC(CCc8cccc9c8n(C)c(=O)n9C8CCC(=O)NC8=O)CC7)CC6)c5)ccc4c23)C(=O)N1] |
| I-105 | 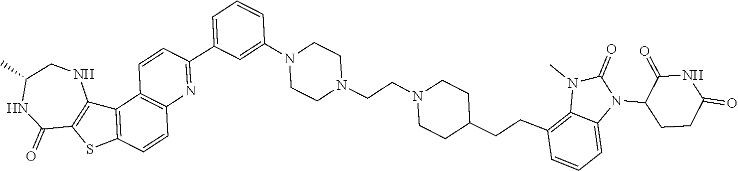 [SMILES: C[C@@H]1CNc2c(sc3ccc4nc(-c5cccc(N6CCN(CCN7CCC(CCc8cccc9c8n(C)c(=O)n9C8CCC(=O)NC8=O)CC7)CC6)c5)ccc4c23)C(=O)N1] |
| I-106 | 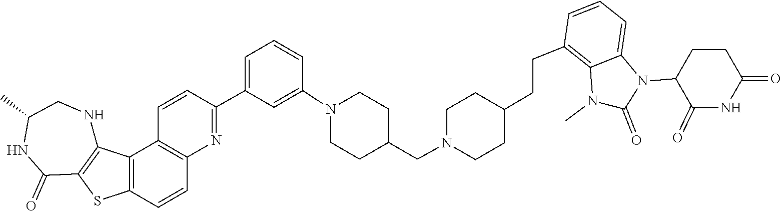 [SMILES: C[C@@H]1CNc2c(sc3ccc4nc(-c5cccc(N6CCC(CN7CCC(CCc8cccc9c8n(C)c(=O)n9C8CCC(=O)NC8=O)CC7)CC6)c5)ccc4c23)C(=O)N1] |
| I-107 | 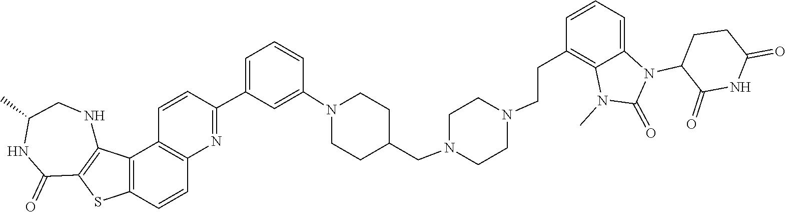 [SMILES: C[C@@H]1CNc2c(sc3ccc4nc(-c5cccc(N6CCC(CN7CCN(CCc8cccc9c8n(C)c(=O)n9C8CCC(=O)NC8=O)CC7)CC6)c5)ccc4c23)C(=O)N1] |
| I-108 | 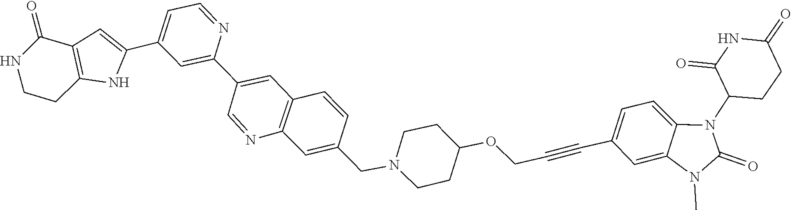 [SMILES: Cn1c(=O)n(C2CCC(=O)NC2=O)c2ccc(C#CCOC3CCN(Cc4ccc5cc(-c6cc(-c7cc8c([nH]7)CCNC8=O)ccn6)cnc5c4)CC3)cc21] |
| I-109 | 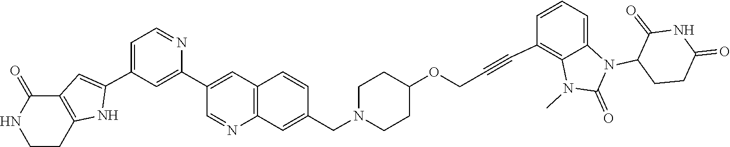 [SMILES: Cn1c(=O)n(C2CCC(=O)NC2=O)c2cccc(C#CCOC3CCN(Cc4ccc5cc(-c6cc(-c7cc8c([nH]7)CCNC8=O)ccn6)cnc5c4)CC3)c21] |
| I-110 | 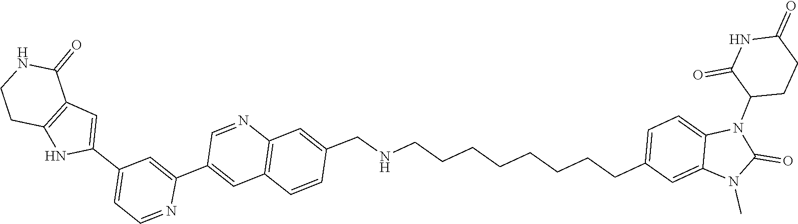 [SMILES: Cn1c(=O)n(C2CCC(=O)NC2=O)c2ccc(CCCCCCCCNCc3ccc4cc(-c5cc(-c6cc7c([nH]6)CCNC7=O)ccn5)cnc4c3)cc21] |
| I-111 | 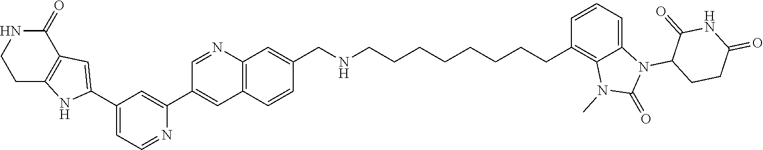 [SMILES: Cn1c(=O)n(C2CCC(=O)NC2=O)c2cccc(CCCCCCCCNCc3ccc4cc(-c5cc(-c6cc7c([nH]6)CCNC7=O)ccn5)cnc4c3)c21] |
| I-112 | 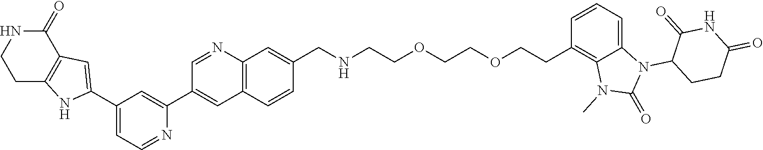 [SMILES: Cn1c(=O)n(C2CCC(=O)NC2=O)c2cccc(CCOCCOCCNCc3ccc4cc(-c5cc(-c6cc7c([nH]6)CCNC7=O)ccn5)cnc4c3)c21] |
| I-113 | 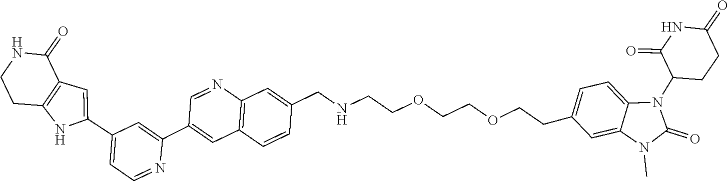 [SMILES: Cn1c(=O)n(C2CCC(=O)NC2=O)c2ccc(CCOCCOCCNCc3ccc4cc(-c5cc(-c6cc7c([nH]6)CCNC7=O)ccn5)cnc4c3)cc21] |
| I-114 | 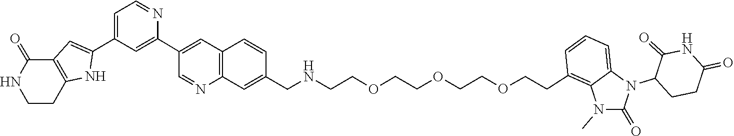 [SMILES: Cn1c(=O)n(C2CCC(=O)NC2=O)c2cccc(CCOCCOCCOCCNCc3ccc4cc(-c5cc(-c6cc7c([nH]6)CCNC7=O)ccn5)cnc4c3)c21] |
| I-115 | 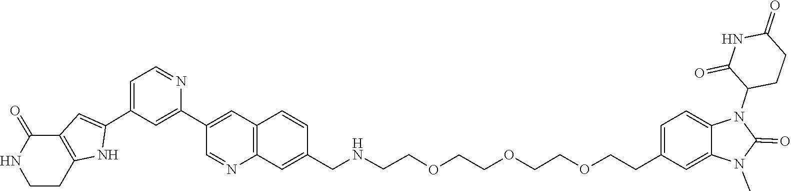 [SMILES: Cn1c(=O)n(C2CCC(=O)NC2=O)c2ccc(CCOCCOCCOCCNCc3ccc4cc(-c5cc(-c6cc7c([nH]6)CCNC7=O)ccn5)cnc4c3)cc21] |
| I-116 | 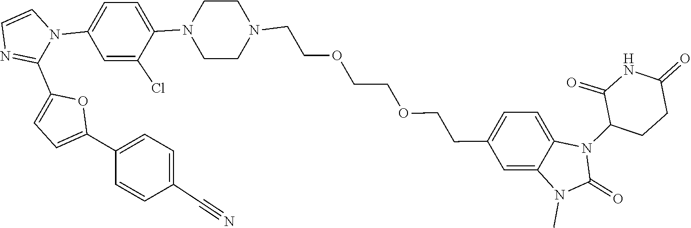 [SMILES: Cn1c(=O)n(C2CCC(=O)NC2=O)c2ccc(CCOCCOCCN3CCN(c4ccc(-n5ccnc5-c5ccc(-c6ccc(C#N)cc6)o5)cc4Cl)CC3)cc21] |
| I-117 | 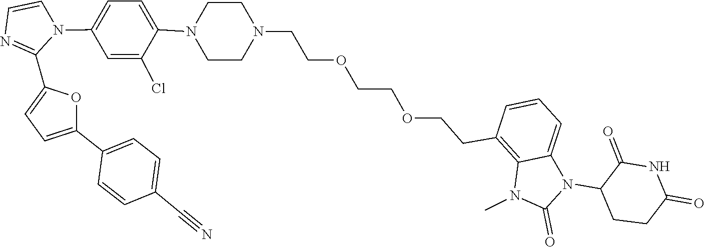 [SMILES: Cn1c(=O)n(C2CCC(=O)NC2=O)c2cccc(CCOCCOCCN3CCN(c4ccc(-n5ccnc5-c5ccc(-c6ccc(C#N)cc6)o5)cc4Cl)CC3)c21] |
| I-118 | 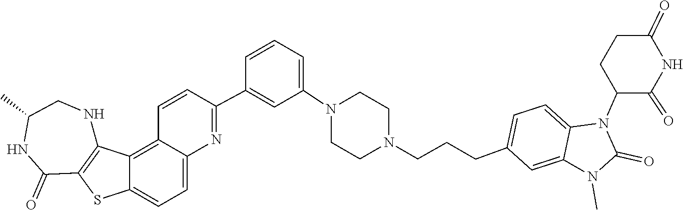 [SMILES: C[C@@H]1CNc2c(sc3ccc4nc(-c5cccc(N6CCN(CCCc7ccc8c(c7)n(C)c(=O)n8C7CCC(=O)NC7=O)CC6)c5)ccc4c23)C(=O)N1] |
| I-119 | 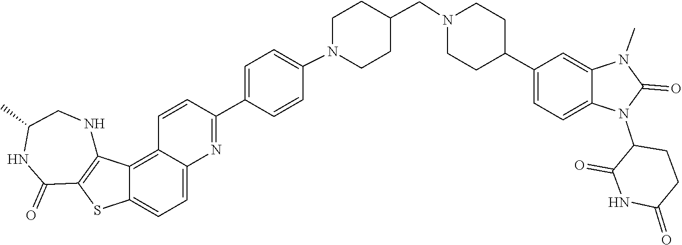 [SMILES: C[C@@H]1CNc2c(sc3ccc4nc(-c5ccc(N6CCC(CN7CCC(c8ccc9c(c8)n(C)c(=O)n9C8CCC(=O)NC8=O)CC7)CC6)cc5)ccc4c23)C(=O)N1] |
| I-120 | 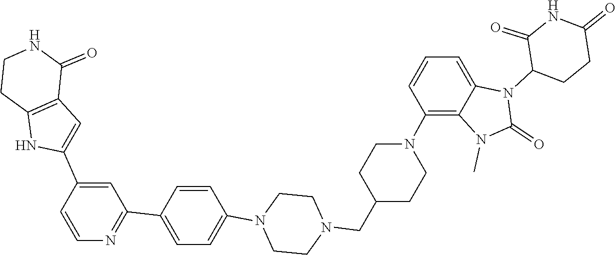 [SMILES: Cn1c(=O)n(C2CCC(=O)NC2=O)c2cccc(N3CCC(CN4CCN(c5ccc(-c6cc(-c7cc8c([nH]7)CCNC8=O)ccn6)cc5)CC4)CC3)c21] |
| I-121 | 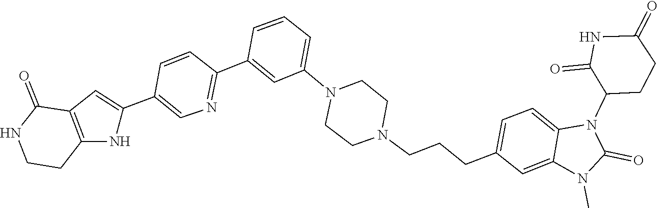 [SMILES: Cn1c(=O)n(C2CCC(=O)NC2=O)c2ccc(CCCN3CCN(c4cccc(-c5ccc(-c6cc7c([nH]6)CCNC7=O)cn5)c4)CC3)cc21] |
| I-122 | 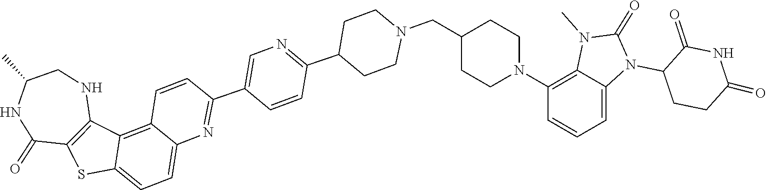 [SMILES: C[C@@H]1CNc2c(sc3ccc4nc(-c5ccc(C6CCN(CC7CCN(c8cccc9c8n(C)c(=O)n9C8CCC(=O)NC8=O)CC7)CC6)nc5)ccc4c23)C(=O)N1] |
| I-123 | 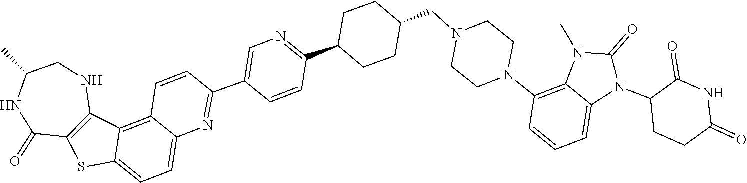 [SMILES: C[C@@H]1CNc2c(sc3ccc4nc(-c5ccc([C@H]6CC[C@H](CN7CCN(c8cccc9c8n(C)c(=O)n9C8CCC(=O)NC8=O)CC7)CC6)nc5)ccc4c23)C(=O)N1] |
| I-124 | 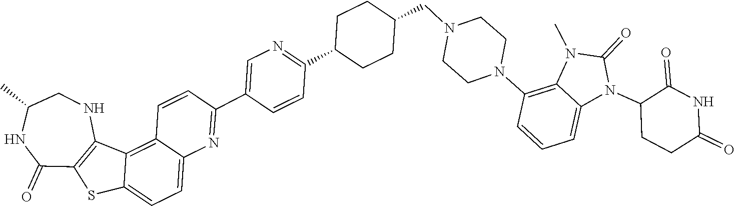 [SMILES: C[C@@H]1CNc2c(sc3ccc4nc(-c5ccc([C@H]6CC[C@@H](CN7CCN(c8cccc9c8n(C)c(=O)n9C8CCC(=O)NC8=O)CC7)CC6)nc5)ccc4c23)C(=O)N1] |
| I-125 | 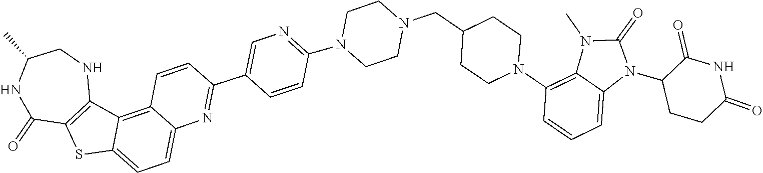 [SMILES: C[C@@H]1CNc2c(sc3ccc4nc(-c5ccc(N6CCN(CC7CCN(c8cccc9c8n(C)c(=O)n9C8CCC(=O)NC8=O)CC7)CC6)nc5)ccc4c23)C(=O)N1] |
| I-126 | 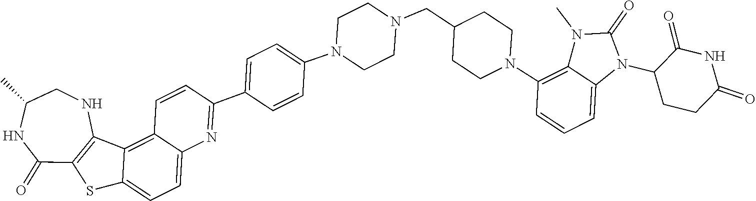 [SMILES: C[C@@H]1CNc2c(sc3ccc4nc(-c5ccc(N6CCN(CC7CCN(c8cccc9c8n(C)c(=O)n9C8CCC(=O)NC8=O)CC7)CC6)cc5)ccc4c23)C(=O)N1] |
| I-127 | 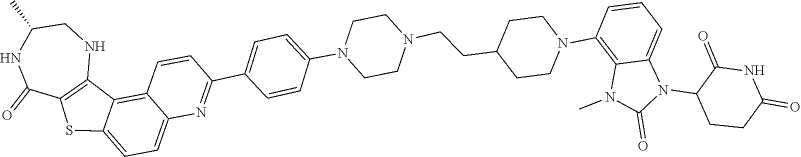 [SMILES: C[C@@H]1CNc2c(sc3ccc4nc(-c5ccc(N6CCN(CCC7CCN(c8cccc9c8n(C)c(=O)n9C8CCC(=O)NC8=O)CC7)CC6)cc5)ccc4c23)C(=O)N1] |
| I-128 | 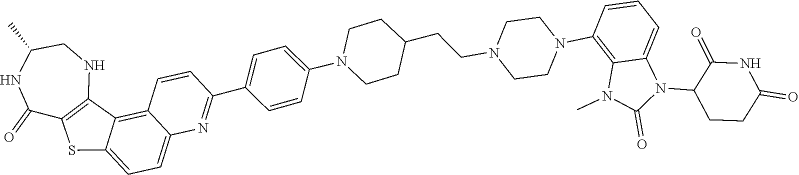 [SMILES: C[C@@H]1CNc2c(sc3ccc4nc(-c5ccc(N6CCC(CCN7CCN(c8cccc9c8n(C)c(=O)n9C8CCC(=O)NC8=O)CC7)CC6)cc5)ccc4c23)C(=O)N1] |
| I-129 | 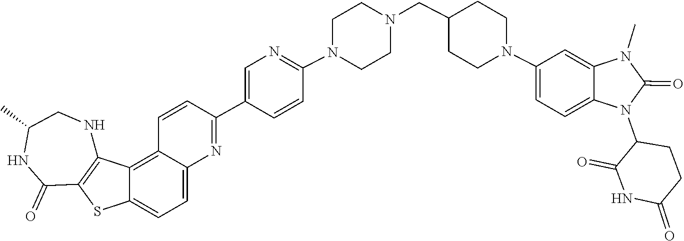 [SMILES: C[C@@H]1CNc2c(sc3ccc4nc(-c5ccc(N6CCN(CC7CCN(c8ccc9c(c8)n(C)c(=O)n9C8CCC(=O)NC8=O)CC7)CC6)nc5)ccc4c23)C(=O)N1] |
| I-130 | 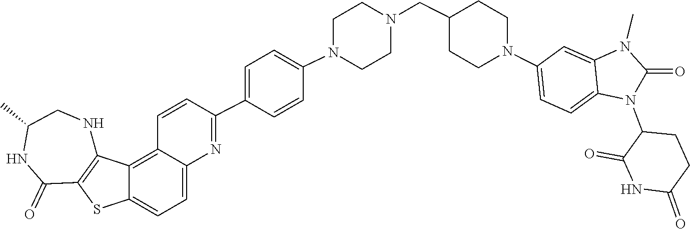 [SMILES: C[C@@H]1CNc2c(sc3ccc4nc(-c5ccc(N6CCN(CC7CCN(c8ccc9c(c8)n(C)c(=O)n9C8CCC(=O)NC8=O)CC7)CC6)cc5)ccc4c23)C(=O)N1] |
| I-131 | 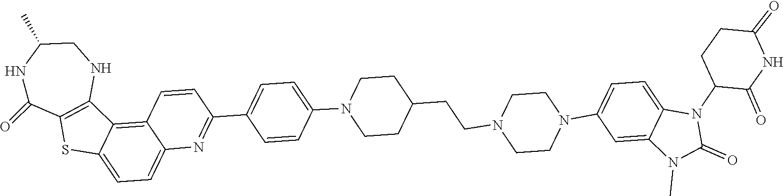 [SMILES: C[C@@H]1CNc2c(sc3ccc4nc(-c5ccc(N6CCC(CCN7CCN(c8ccc9c(c8)n(C)c(=O)n9C8CCC(=O)NC8=O)CC7)CC6)cc5)ccc4c23)C(=O)N1] |
| I-132 | 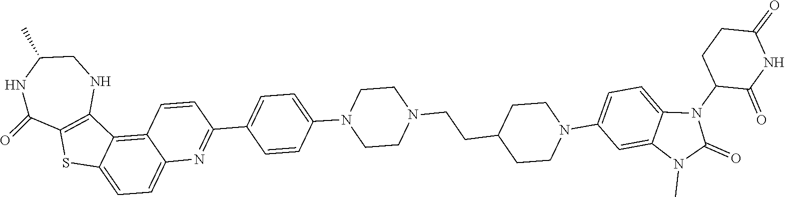 [SMILES: C[C@@H]1CNc2c(sc3ccc4nc(-c5ccc(N6CCN(CCC7CCN(c8ccc9c(c8)n(C)c(=O)n9C8CCC(=O)NC8=O)CC7)CC6)cc5)ccc4c23)C(=O)N1] |
| I-133 | 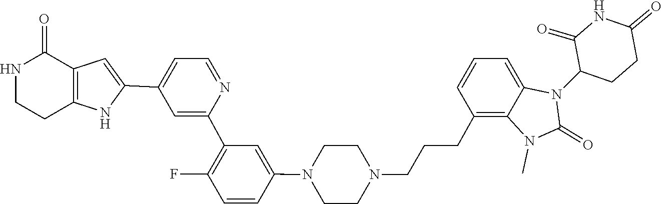 [SMILES: Cn1c(=O)n(C2CCC(=O)NC2=O)c2cccc(CCCN3CCN(c4ccc(F)c(-c5cc(-c6cc7c([nH]6)CCNC7=O)ccn5)c4)CC3)c21] |
| I-134 | 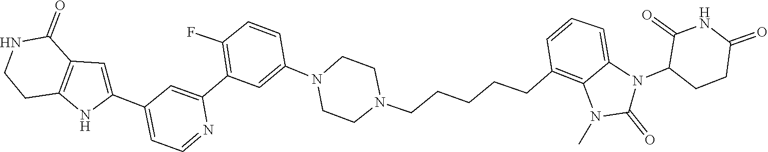 [SMILES: Cn1c(=O)n(C2CCC(=O)NC2=O)c2cccc(CCCCCN3CCN(c4ccc(F)c(-c5cc(-c6cc7c([nH]6)CCNC7=O)ccn5)c4)CC3)c21] |
| I-135 | 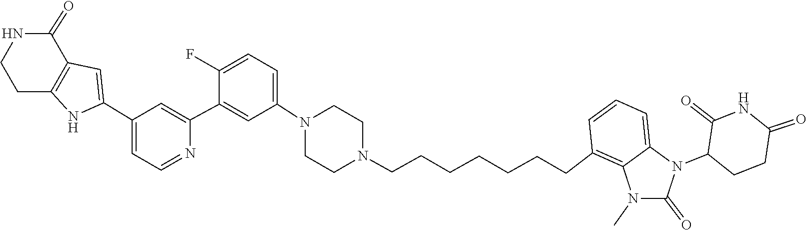 [SMILES: Cn1c(=O)n(C2CCC(=O)NC2=O)c2cccc(CCCCCCCN3CCN(c4ccc(F)c(-c5cc(-c6cc7c([nH]6)CCNC7=O)ccn5)c4)CC3)c21] |
| I-136 | 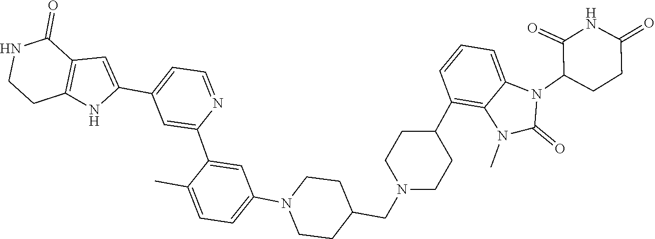 [SMILES: Cc1ccc(N2CCC(CN3CCC(c4cccc5c4n(C)c(=O)n5C4CCC(=O)NC4=O)CC3)CC2)cc1-c1cc(-c2cc3c([nH]2)CCNC3=O)ccn1] |
| I-137 | 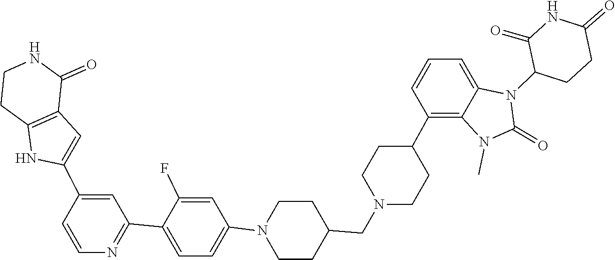 [SMILES: Cn1c(=O)n(C2CCC(=O)NC2=O)c2cccc(C3CCN(CC4CCN(c5ccc(-c6cc(-c7cc8c([nH]7)CCNC8=O)ccn6)c(F)c5)CC4)CC3)c21] |
| I-138 | 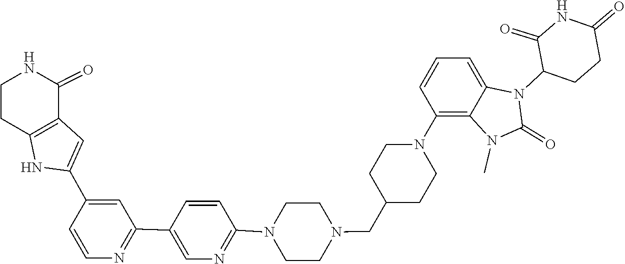 [SMILES: Cn1c(=O)n(C2CCC(=O)NC2=O)c2cccc(N3CCC(CN4CCN(c5ccc(-c6cc(-c7cc8c([nH]7)CCNC8=O)ccn6)cn5)CC4)CC3)c21] |
| I-139 | 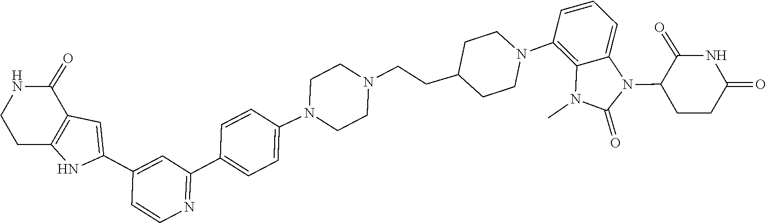 [SMILES: Cn1c(=O)n(C2CCC(=O)NC2=O)c2cccc(N3CCC(CCN4CCN(c5ccc(-c6cc(-c7cc8c([nH]7)CCNC8=O)ccn6)cc5)CC4)CC3)c21] |
| I-140 | 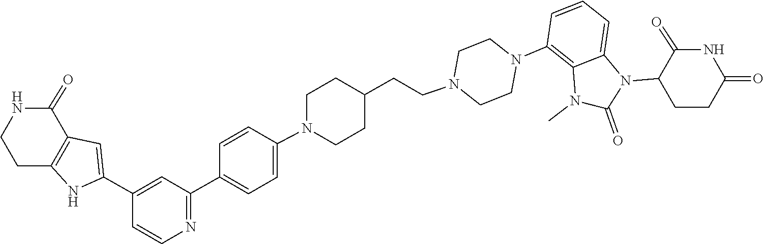 [SMILES: Cn1c(=O)n(C2CCC(=O)NC2=O)c2cccc(N3CCN(CCC4CCN(c5ccc(-c6cc(-c7cc8c([nH]7)CCNC8=O)ccn6)cc5)CC4)CC3)c21] |
| I-141 | 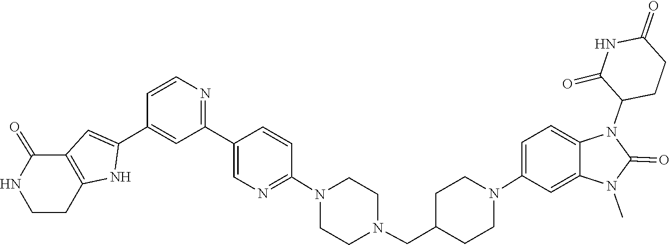 [SMILES: Cn1c(=O)n(C2CCC(=O)NC2=O)c2ccc(N3CCC(CN4CCN(c5ccc(-c6cc(-c7cc8c([nH]7)CCNC8=O)ccn6)cn5)CC4)CC3)cc21] |
| I-142 | 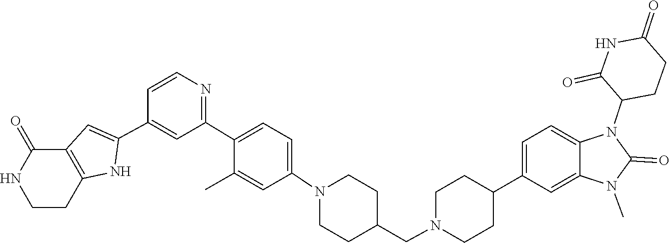 [SMILES: Cc1cc(N2CCC(CN3CCC(c4ccc5c(c4)n(C)c(=O)n5C4CCC(=O)NC4=O)CC3)CC2)ccc1-c1cc(-c2cc3c([nH]2)CCNC3=O)ccn1] |
| I-143 | 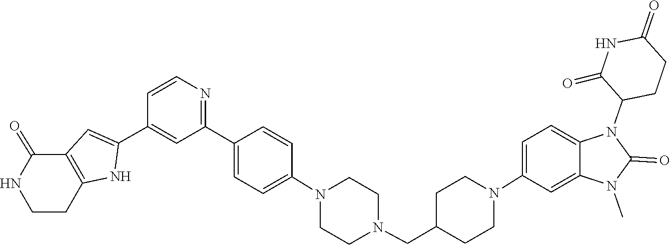 [SMILES: Cn1c(=O)n(C2CCC(=O)NC2=O)c2ccc(N3CCC(CN4CCN(c5ccc(-c6cc(-c7cc8c([nH]7)CCNC8=O)ccn6)cc5)CC4)CC3)cc21] |
| I-144 | 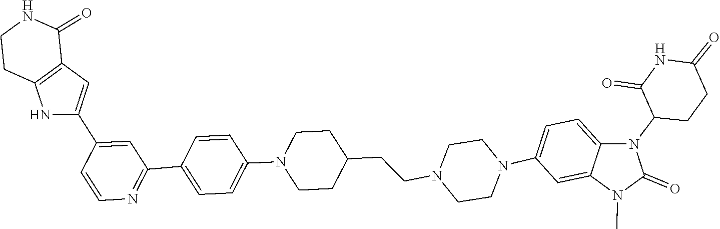 [SMILES: Cn1c(=O)n(C2CCC(=O)NC2=O)c2ccc(N3CCN(CCC4CCN(c5ccc(-c6cc(-c7cc8c([nH]7)CCNC8=O)ccn6)cc5)CC4)CC3)cc21] |
| I-145 | 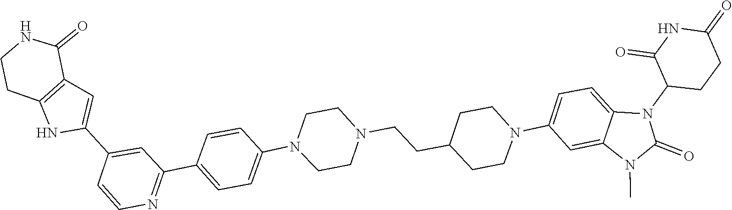 [SMILES: Cn1c(=O)n(C2CCC(=O)NC2=O)c2ccc(N3CCC(CCN4CCN(c5ccc(-c6cc(-c7cc8c([nH]7)CCNC8=O)ccn6)cc5)CC4)CC3)cc21] |
| I-146 | 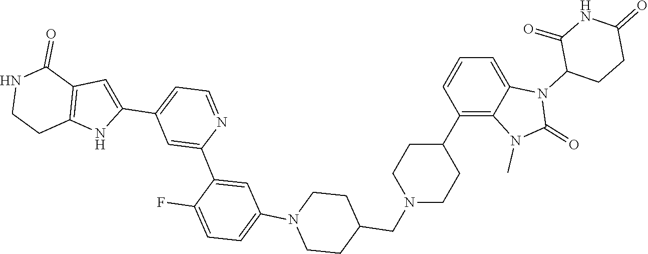 [SMILES: Cn1c(=O)n(C2CCC(=O)NC2=O)c2cccc(C3CCN(CC4CCN(c5ccc(F)c(-c6cc(-c7cc8c([nH]7)CCNC8=O)ccn6)c5)CC4)CC3)c21] |
| I-147 | 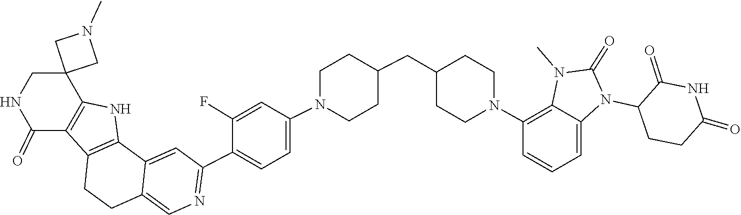 [SMILES: CN1CC2(CNC(=O)c3c2[nH]c2c3CCc3cnc(-c4ccc(N5CCC(CC6CCN(c7cccc8c7n(C)c(=O)n8C7CCC(=O)NC7=O)CC6)CC5)cc4F)cc3-2)C1] |
| I-148 | 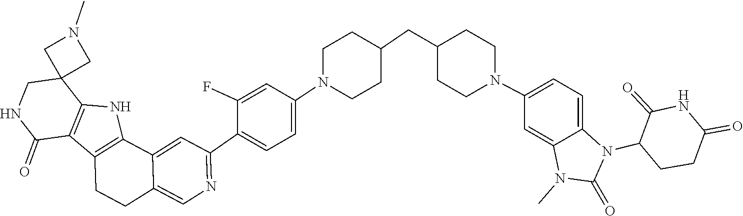 [SMILES: CN1CC2(CNC(=O)c3c2[nH]c2c3CCc3cnc(-c4ccc(N5CCC(CC6CCN(c7ccc8c(c7)n(C)c(=O)n8C7CCC(=O)NC7=O)CC6)CC5)cc4F)cc3-2)C1] |
| I-149 | 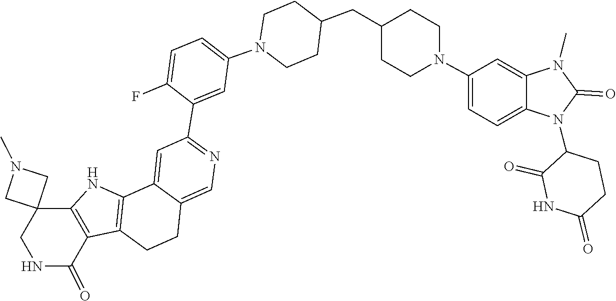 [SMILES: CN1CC2(CNC(=O)c3c2[nH]c2c3CCc3cnc(-c4cc(N5CCC(CC6CCN(c7ccc8c(c7)n(C)c(=O)n8C7CCC(=O)NC7=O)CC6)CC5)ccc4F)cc3-2)C1] |
| I-150 | 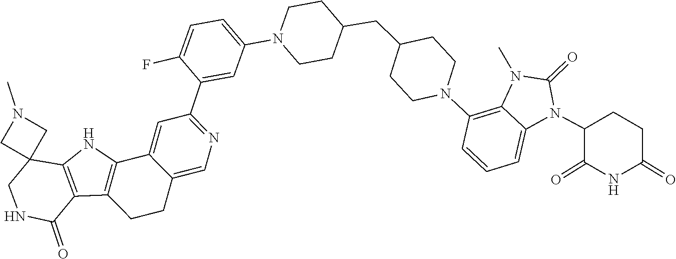 [SMILES: CN1CC2(CNC(=O)c3c2[nH]c2c3CCc3cnc(-c4cc(N5CCC(CC6CCN(c7cccc8c7n(C)c(=O)n8C7CCC(=O)NC7=O)CC6)CC5)ccc4F)cc3-2)C1] |
| I-151 | 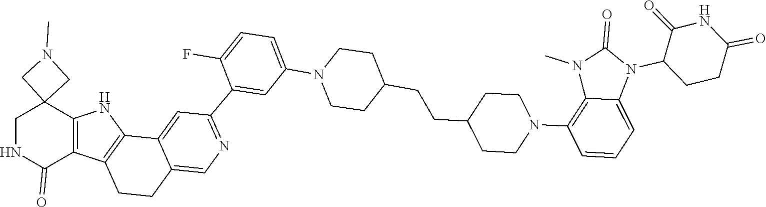 [SMILES: CN1CC2(CNC(=O)c3c2[nH]c2c3CCc3cnc(-c4cc(N5CCC(CCC6CCN(c7cccc8c7n(C)c(=O)n8C7CCC(=O)NC7=O)CC6)CC5)ccc4F)cc3-2)C1] |
| I-152 | 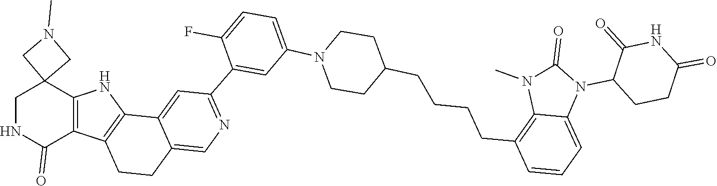 [SMILES: CN1CC2(CNC(=O)c3c2[nH]c2c3CCc3cnc(-c4cc(N5CCC(CCCCc6cccc7c6n(C)c(=O)n7C6CCC(=O)NC6=O)CC5)ccc4F)cc3-2)C1] |
| I-153 | 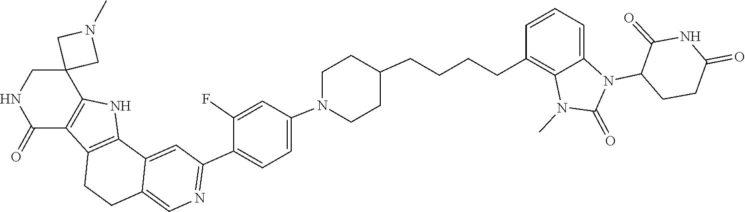 [SMILES: CN1CC2(CNC(=O)c3c2[nH]c2c3CCc3cnc(-c4ccc(N5CCC(CCCCc6cccc7c6n(C)c(=O)n7C6CCC(=O)NC6=O)CC5)cc4F)cc3-2)C1] |
| I-154 | 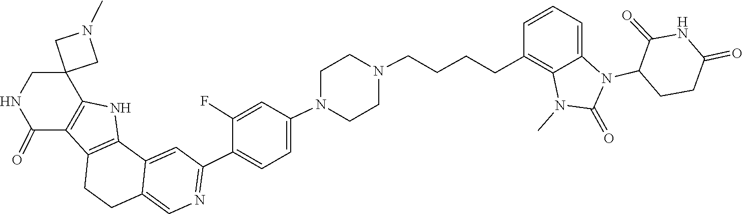 [SMILES: CN1CC2(CNC(=O)c3c2[nH]c2c3CCc3cnc(-c4ccc(N5CCN(CCCCc6cccc7c6n(C)c(=O)n7C6CCC(=O)NC6=O)CC5)cc4F)cc3-2)C1] |
| I-155 | 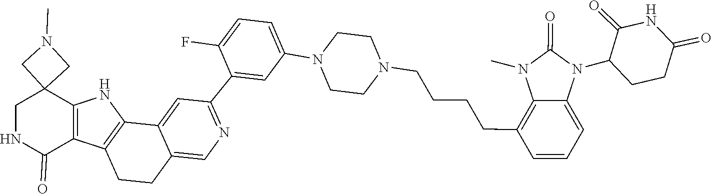 [SMILES: CN1CC2(CNC(=O)c3c2[nH]c2c3CCc3cnc(-c4cc(N5CCN(CCCCc6cccc7c6n(C)c(=O)n7C6CCC(=O)NC6=O)CC5)ccc4F)cc3-2)C1] |
| I-156 | 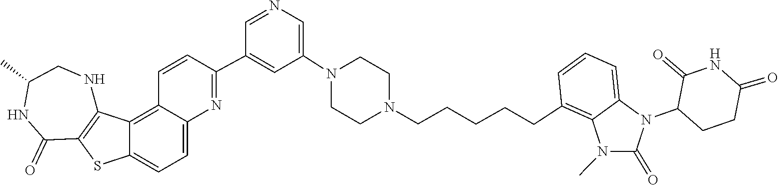 [SMILES: C[C@@H]1CNc2c(sc3ccc4nc(-c5cncc(N6CCN(CCCCCc7cccc8c7n(C)c(=O)n8C7CCC(=O)NC7=O)CC6)c5)ccc4c23)C(=O)N1] |
| I-157 | 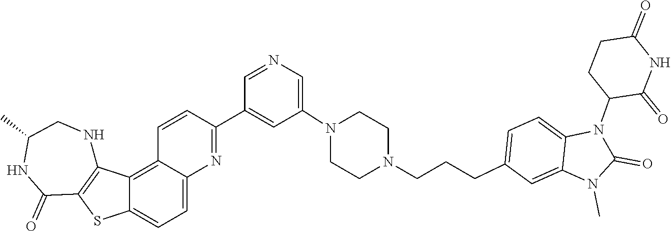 [SMILES: C[C@@H]1CNc2c(sc3ccc4nc(-c5cncc(N6CCN(CCCc7ccc8c(c7)n(C)c(=O)n8C7CCC(=O)NC7=O)CC6)c5)ccc4c23)C(=O)N1] |
| I-158 | 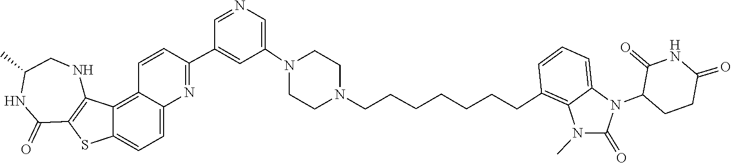 [SMILES: C[C@@H]1CNc2c(sc3ccc4nc(-c5cncc(N6CCN(CCCCCCCc7cccc8c7n(C)c(=O)n8C7CCC(=O)NC7=O)CC6)c5)ccc4c23)C(=O)N1] |
| I-159 | 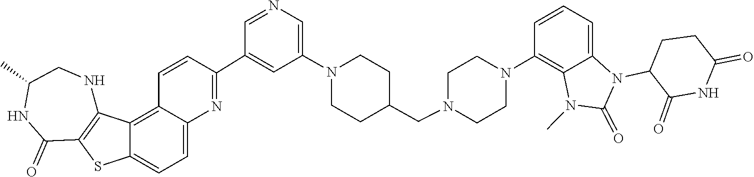 [SMILES: C[C@@H]1CNc2c(sc3ccc4nc(-c5cncc(N6CCC(CN7CCN(c8cccc9c8n(C)c(=O)n9C8CCC(=O)NC8=O)CC7)CC6)c5)ccc4c23)C(=O)N1] |
| I-160 | 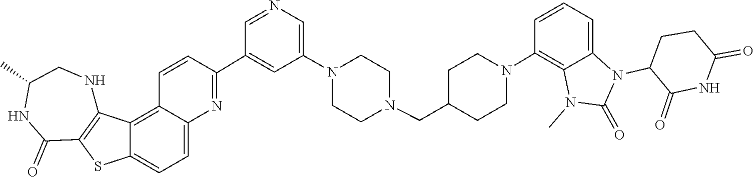 [SMILES: C[C@@H]1CNc2c(sc3ccc4nc(-c5cncc(N6CCN(CC7CCN(c8cccc9c8n(C)c(=O)n9C8CCC(=O)NC8=O)CC7)CC6)c5)ccc4c23)C(=O)N1] |
| I-161 | 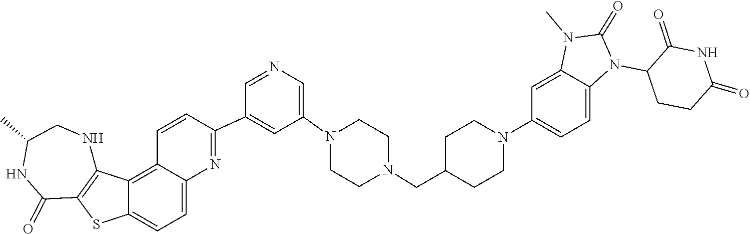 [SMILES: C[C@@H]1CNc2c(sc3ccc4nc(-c5cncc(N6CCN(CC7CCN(c8ccc9c(c8)n(C)c(=O)n9C8CCC(=O)NC8=O)CC7)CC6)c5)ccc4c23)C(=O)N1] |
| I-162 | 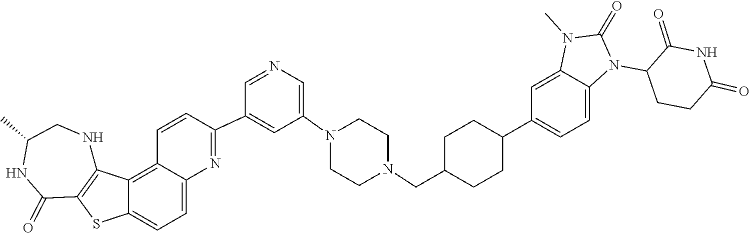 [SMILES: C[C@@H]1CNc2c(sc3ccc4nc(-c5cncc(N6CCN(CC7CCC(c8ccc9c(c8)n(C)c(=O)n9C8CCC(=O)NC8=O)CC7)CC6)c5)ccc4c23)C(=O)N1] |
| I-163 | 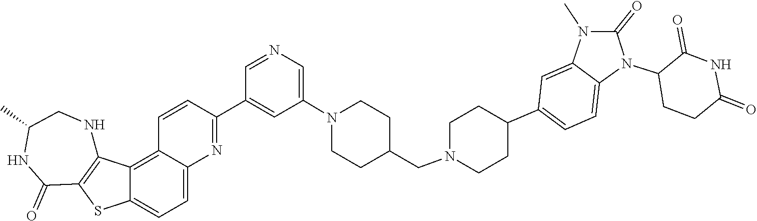 [SMILES: C[C@@H]1CNc2c(sc3ccc4nc(-c5cncc(N6CCC(CN7CCC(c8ccc9c(c8)n(C)c(=O)n9C8CCC(=O)NC8=O)CC7)CC6)c5)ccc4c23)C(=O)N1] |
| I-164 | 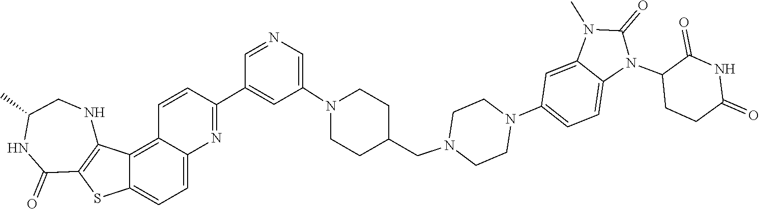 [SMILES: C[C@@H]1CNc2c(sc3ccc4nc(-c5cncc(N6CCC(CN7CCN(c8ccc9c(c8)n(C)c(=O)n9C8CCC(=O)NC8=O)CC7)CC6)c5)ccc4c23)C(=O)N1] |
| I-165 | 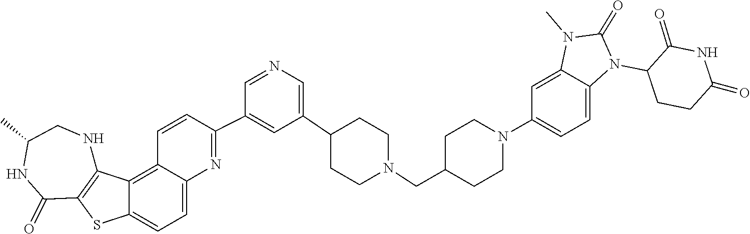 [SMILES: C[C@@H]1CNc2c(sc3ccc4nc(-c5cncc(C6CCN(CC7CCN(c8ccc9c(c8)n(C)c(=O)n9C8CCC(=O)NC8=O)CC7)CC6)c5)ccc4c23)C(=O)N1] |
| I-166 | 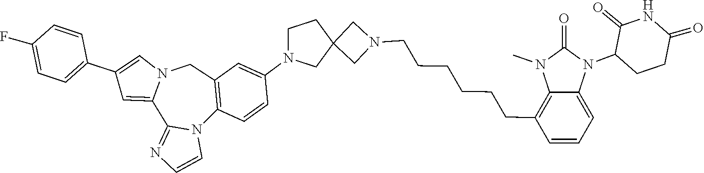 [SMILES: Cn1c(=O)n(C2CCC(=O)NC2=O)c2cccc(CCCCCCN3CC4(CCN(c5ccc6c(c5)Cn5cc(-c7ccc(F)cc7)cc5-c5nccn5-6)C4)C3)c21] |
| I-167 | 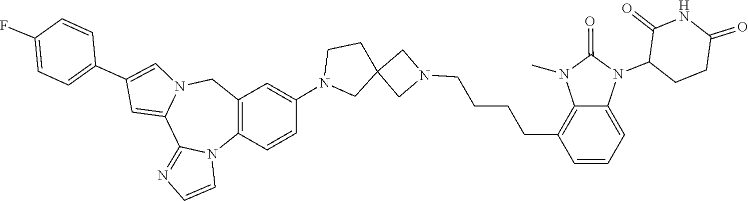 [SMILES: Cn1c(=O)n(C2CCC(=O)NC2=O)c2cccc(CCCCN3CC4(CCN(c5ccc6c(c5)Cn5cc(-c7ccc(F)cc7)cc5-c5nccn5-6)C4)C3)c21] |
| I-168 | 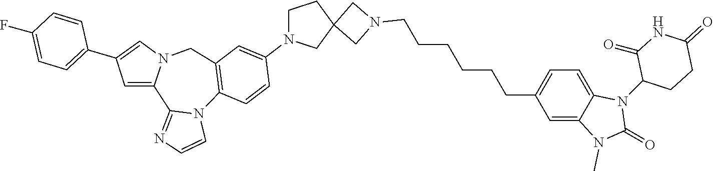 [SMILES: Cn1c(=O)n(C2CCC(=O)NC2=O)c2ccc(CCCCCCN3CC4(CCN(c5ccc6c(c5)Cn5cc(-c7ccc(F)cc7)cc5-c5nccn5-6)C4)C3)cc21] |
| I-169 | 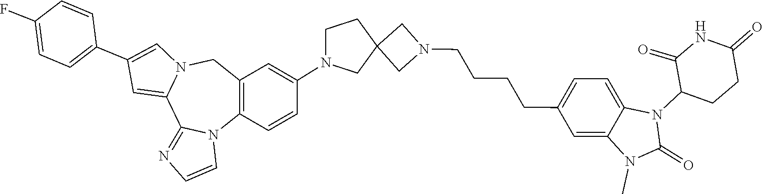 [SMILES: Cn1c(=O)n(C2CCC(=O)NC2=O)c2ccc(CCCCN3CC4(CCN(c5ccc6c(c5)Cn5cc(-c7ccc(F)cc7)cc5-c5nccn5-6)C4)C3)cc21] |
| I-170 | 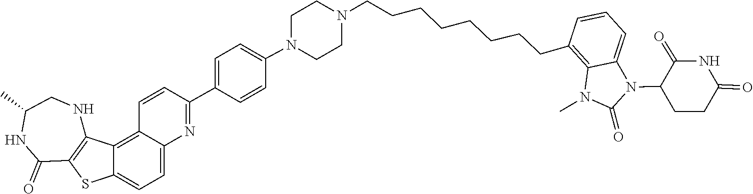 [SMILES: C[C@@H]1CNc2c(sc3ccc4nc(-c5ccc(N6CCN(CCCCCCCCc7cccc8c7n(C)c(=O)n8C7CCC(=O)NC7=O)CC6)cc5)ccc4c23)C(=O)N1] |
| I-171 | 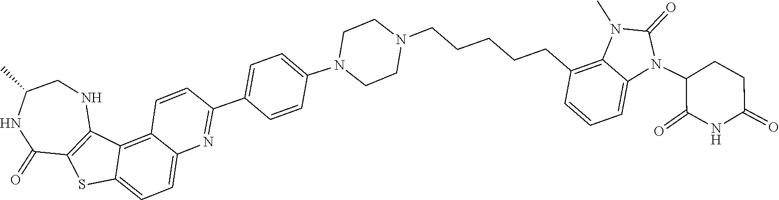 [SMILES: C[C@@H]1CNc2c(sc3ccc4nc(-c5ccc(N6CCN(CCCCCc7cccc8c7n(C)c(=O)n8C7CCC(=O)NC7=O)CC6)cc5)ccc4c23)C(=O)N1] |
| I-172 | 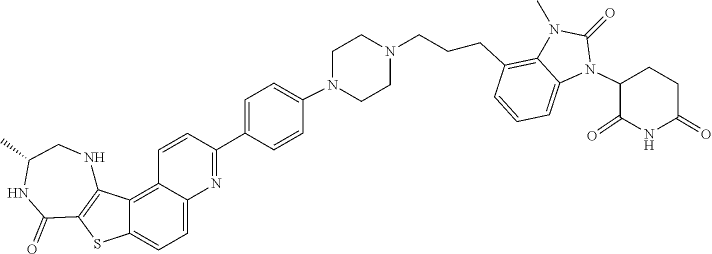 [SMILES: C[C@@H]1CNc2c(sc3ccc4nc(-c5ccc(N6CCN(CCCc7cccc8c7n(C)c(=O)n8C7CCC(=O)NC7=O)CC6)cc5)ccc4c23)C(=O)N1] |
| I-173 | 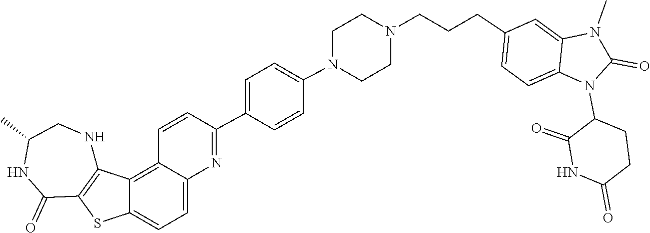 [SMILES: C[C@@H]1CNc2c(sc3ccc4nc(-c5ccc(N6CCN(CCCc7ccc8c(c7)n(C)c(=O)n8C7CCC(=O)NC7=O)CC6)cc5)ccc4c23)C(=O)N1] |
| I-174 | 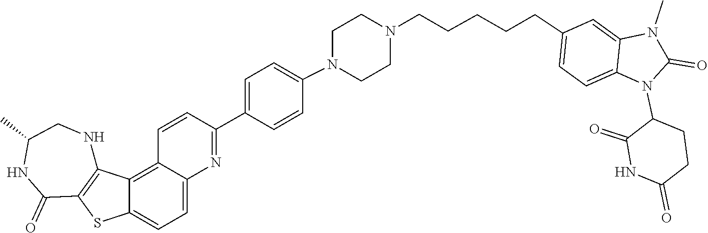 [SMILES: C[C@@H]1CNc2c(sc3ccc4nc(-c5ccc(N6CCN(CCCCCc7ccc8c(c7)n(C)c(=O)n8C7CCC(=O)NC7=O)CC6)cc5)ccc4c23)C(=O)N1] |
| I-175 | 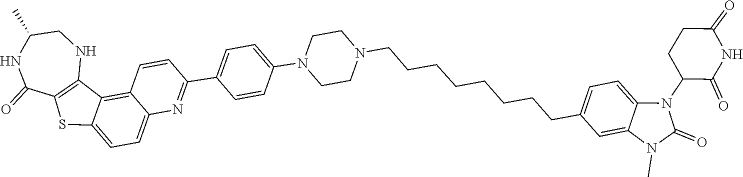 [SMILES: C[C@@H]1CNc2c(sc3ccc4nc(-c5ccc(N6CCN(CCCCCCCCc7ccc8c(c7)n(C)c(=O)n8C7CCC(=O)NC7=O)CC6)cc5)ccc4c23)C(=O)N1] |
| I-176 | 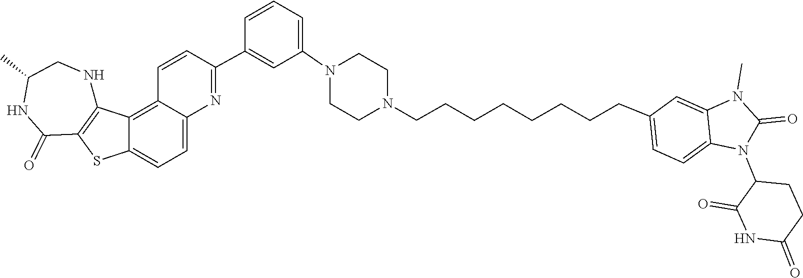 [SMILES: C[C@@H]1CNc2c(sc3ccc4nc(-c5cccc(N6CCN(CCCCCCCCc7ccc8c(c7)n(C)c(=O)n8C7CCC(=O)NC7=O)CC6)c5)ccc4c23)C(=O)N1] |
| I-177 | 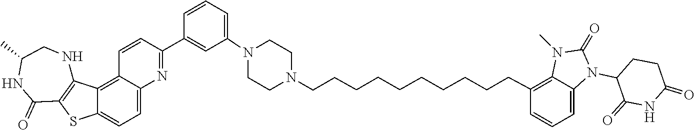 [SMILES: C[C@@H]1CNc2c(sc3ccc4nc(-c5cccc(N6CCN(CCCCCCCCCCc7cccc8c7n(C)c(=O)n8C7CCC(=O)NC7=O)CC6)c5)ccc4c23)C(=O)N1] |
| I-178 | 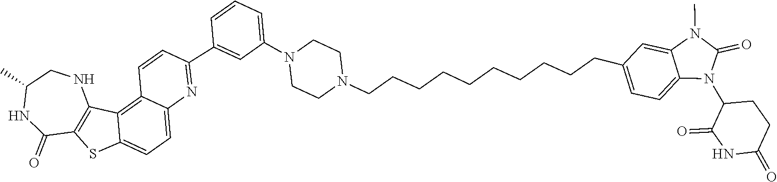 [SMILES: C[C@@H]1CNc2c(sc3ccc4nc(-c5cccc(N6CCN(CCCCCCCCCCc7ccc8c(c7)n(C)c(=O)n8C7CCC(=O)NC7=O)CC6)c5)ccc4c23)C(=O)N1] |
| I-179 | 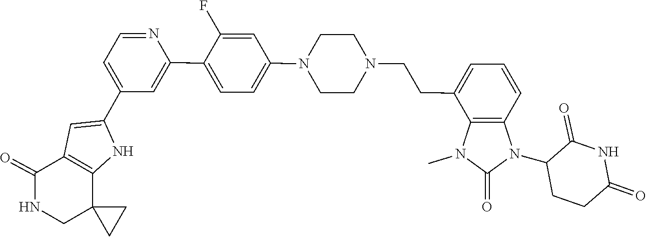 [SMILES: Cn1c(=O)n(C2CCC(=O)NC2=O)c2cccc(CCN3CCN(c4ccc(-c5cc(-c6cc7c([nH]6)C6(CC6)CNC7=O)ccn5)c(F)c4)CC3)c21] |
| I-180 | 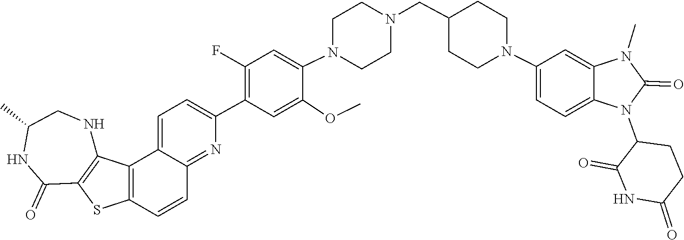 [SMILES: COc1cc(-c2ccc3c(ccc4sc5c(c43)NC[C@@H](C)NC5=O)n2)c(F)cc1N1CCN(CC2CCN(c3ccc4c(c3)n(C)c(=O)n4C3CCC(=O)NC3=O)CC2)CC1] |
| I-181 | 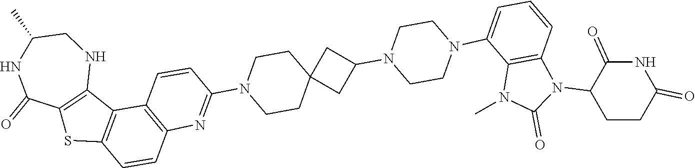 [SMILES: C[C@@H]1CNc2c(sc3ccc4nc(N5CCC6(CC5)CC(N5CCN(c7cccc8c7n(C)c(=O)n8C7CCC(=O)NC7=O)CC5)C6)ccc4c23)C(=O)N1] |
| I-182 | 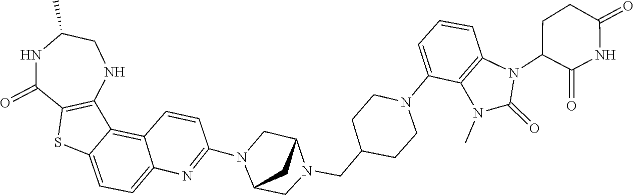 [SMILES: C[C@@H]1CNc2c(sc3ccc4nc(N5C[C@H]6C[C@@H]5CN6CC5CCN(c6cccc7c6n(C)c(=O)n7C6CCC(=O)NC6=O)CC5)ccc4c23)C(=O)N1] |
| I-183 | 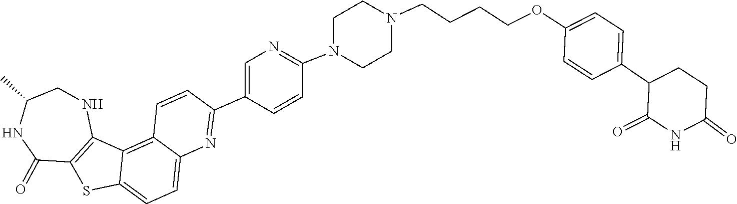 [SMILES: C[C@@H]1CNc2c(sc3ccc4nc(-c5ccc(N6CCN(CCCCOc7ccc(C8CCC(=O)NC8=O)cc7)CC6)nc5)ccc4c23)C(=O)N1] |
| I-184 | 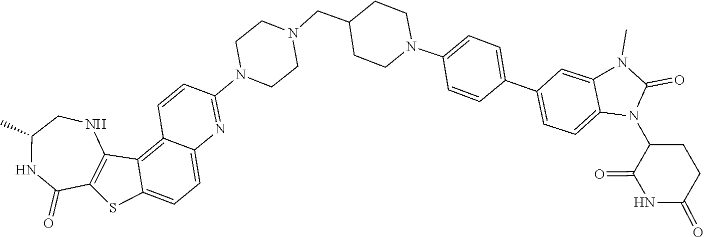 [SMILES: C[C@@H]1CNc2c(sc3ccc4nc(N5CCN(CC6CCN(c7ccc(-c8ccc9c(c8)n(C)c(=O)n9C8CCC(=O)NC8=O)cc7)CC6)CC5)ccc4c23)C(=O)N1] |
| I-185 | 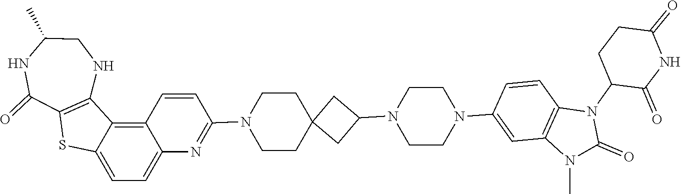 [SMILES: C[C@@H]1CNc2c(sc3ccc4nc(N5CCC6(CC5)CC(N5CCN(c7ccc8c(c7)n(C)c(=O)n8C7CCC(=O)NC7=O)CC5)C6)ccc4c23)C(=O)N1] |
| I-186 | 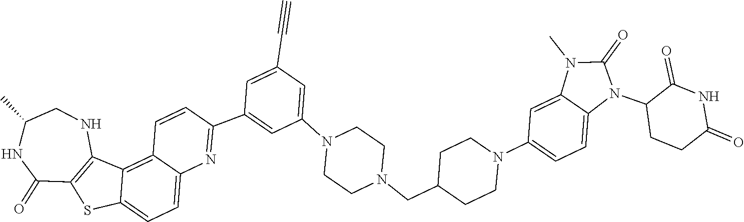 [SMILES: C#Cc1cc(-c2ccc3c(ccc4sc5c(c43)NC[C@@H](C)NC5=O)n2)cc(N2CCN(CC3CCN(c4ccc5c(c4)n(C)c(=O)n5C4CCC(=O)NC4=O)CC3)CC2)c1] |
| I-187 | 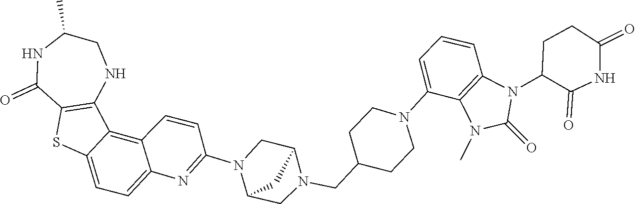 [SMILES: C[C@@H]1CNc2c(sc3ccc4nc(N5C[C@@H]6C[C@H]5CN6CC5CCN(c6cccc7c6n(C)c(=O)n7C6CCC(=O)NC6=O)CC5)ccc4c23)C(=O)N1] |
| I-188 | 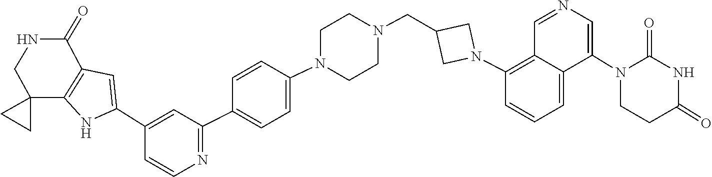 [SMILES: O=C1CCN(c2cncc3c(N4CC(CN5CCN(c6ccc(-c7cc(-c8cc9c([nH]8)C8(CC8)CNC9=O)ccn7)cc6)CC5)C4)cccc23)C(=O)N1] |
| I-189 | 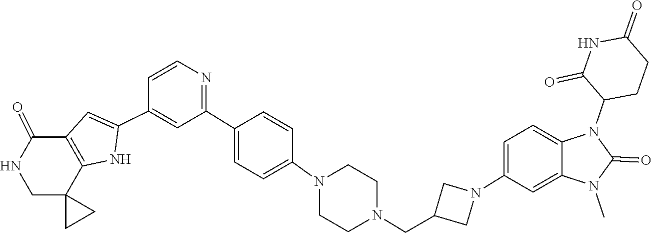 [SMILES: Cn1c(=O)n(C2CCC(=O)NC2=O)c2ccc(N3CC(CN4CCN(c5ccc(-c6cc(-c7cc8c([nH]7)C7(CC7)CNC8=O)ccn6)cc5)CC4)C3)cc21] |
| I-190 | 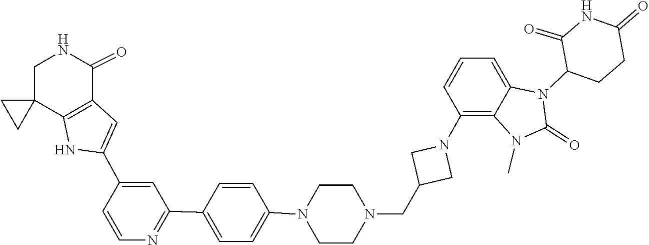 [SMILES: Cn1c(=O)n(C2CCC(=O)NC2=O)c2cccc(N3CC(CN4CCN(c5ccc(-c6cc(-c7cc8c([nH]7)C7(CC7)CNC8=O)ccn6)cc5)CC4)C3)c21] |
| I-191 | 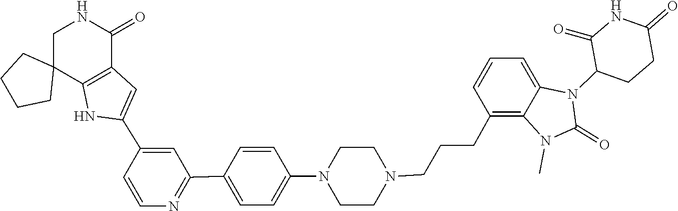 [SMILES: Cn1c(=O)n(C2CCC(=O)NC2=O)c2cccc(CCCN3CCN(c4ccc(-c5cc(-c6cc7c([nH]6)C6(CCCC6)CNC7=O)ccn5)cc4)CC3)c21] |
| I-192 | 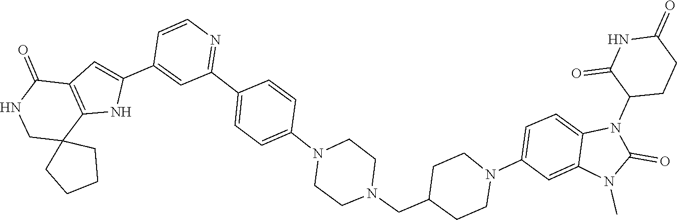 [SMILES: Cn1c(=O)n(C2CCC(=O)NC2=O)c2ccc(N3CCC(CN4CCN(c5ccc(-c6cc(-c7cc8c([nH]7)C7(CCCC7)CNC8=O)ccn6)cc5)CC4)CC3)cc21] |
| I-193 | 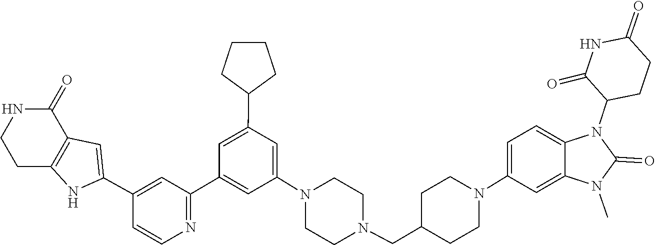 [SMILES: Cn1c(=O)n(C2CCC(=O)NC2=O)c2ccc(N3CCC(CN4CCN(c5cc(-c6cc(-c7cc8c([nH]7)CCNC8=O)ccn6)cc(C6CCCC6)c5)CC4)CC3)cc21] |
| I-194 | 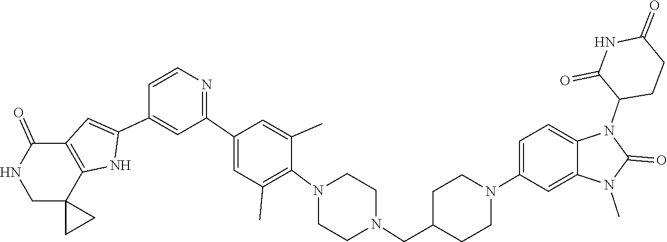 [SMILES: Cc1cc(-c2cc(-c3cc4c([nH]3)C3(CC3)CNC4=O)ccn2)cc(C)c1N1CCN(CC2CCN(c3ccc4c(c3)n(C)c(=O)n4C3CCC(=O)NC3=O)CC2)CC1] |
| I-195 | 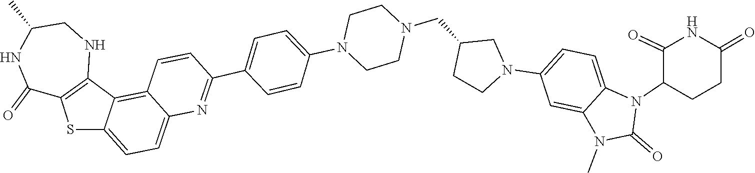 [SMILES: C[C@@H]1CNc2c(sc3ccc4nc(-c5ccc(N6CCN(C[C@H]7CCN(c8ccc9c(c8)n(C)c(=O)n9C8CCC(=O)NC8=O)C7)CC6)cc5)ccc4c23)C(=O)N1] |
| I-196 | 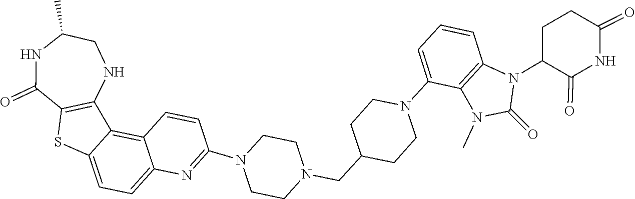 [SMILES: C[C@@H]1CNc2c(sc3ccc4nc(N5CCN(CC6CCN(c7cccc8c7n(C)c(=O)n8C7CCC(=O)NC7=O)CC6)CC5)ccc4c23)C(=O)N1] |
| I-197 | 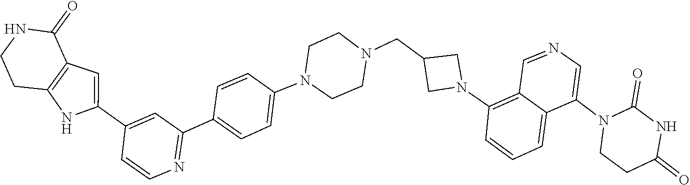 [SMILES: O=C1CCN(c2cncc3c(N4CC(CN5CCN(c6ccc(-c7cc(-c8cc9c([nH]8)CCNC9=O)ccn7)cc6)CC5)C4)cccc23)C(=O)N1] |
| I-198 | 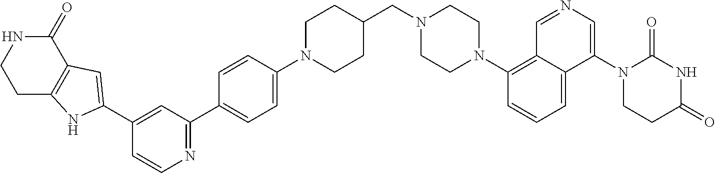 [SMILES: O=C1CCN(c2cncc3c(N4CCN(CC5CCN(c6ccc(-c7cc(-c8cc9c([nH]8)CCNC9=O)ccn7)cc6)CC5)CC4)cccc23)C(=O)N1] |
| I-199 | 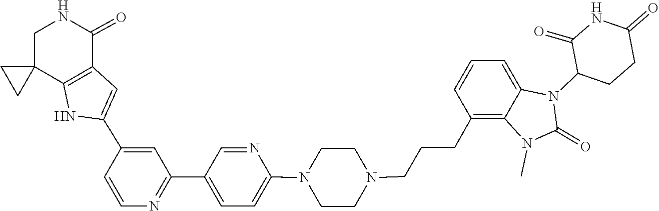 [SMILES: Cn1c(=O)n(C2CCC(=O)NC2=O)c2cccc(CCCN3CCN(c4ccc(-c5cc(-c6cc7c([nH]6)C6(CC6)CNC7=O)ccn5)cn4)CC3)c21] |
| I-200 | 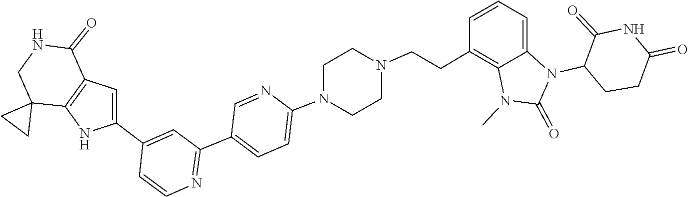 [SMILES: Cn1c(=O)n(C2CCC(=O)NC2=O)c2cccc(CCN3CCN(c4ccc(-c5cc(-c6cc7c([nH]6)C6(CC6)CNC7=O)ccn5)cn4)CC3)c21] |
| I-201 | 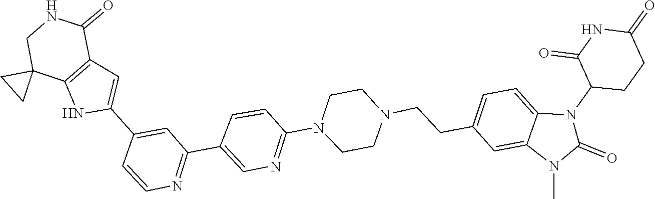 [SMILES: Cn1c(=O)n(C2CCC(=O)NC2=O)c2ccc(CCN3CCN(c4ccc(-c5cc(-c6cc7c([nH]6)C6(CC6)CNC7=O)ccn5)cn4)CC3)cc21] |
| I-202 | 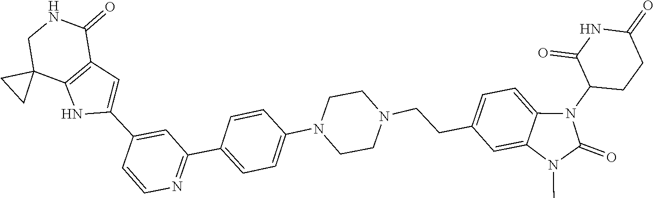 [SMILES: Cn1c(=O)n(C2CCC(=O)NC2=O)c2ccc(CCN3CCN(c4ccc(-c5cc(-c6cc7c([nH]6)C6(CC6)CNC7=O)ccn5)cc4)CC3)cc21] |
| I-203 | 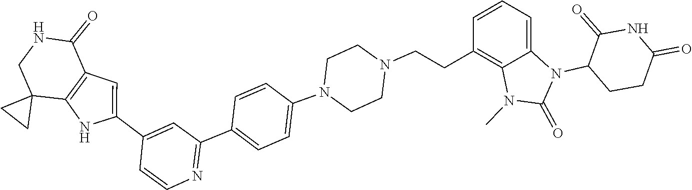 [SMILES: Cn1c(=O)n(C2CCC(=O)NC2=O)c2cccc(CCN3CCN(c4ccc(-c5cc(-c6cc7c([nH]6)C6(CC6)CNC7=O)ccn5)cc4)CC3)c21] |
| I-204 | 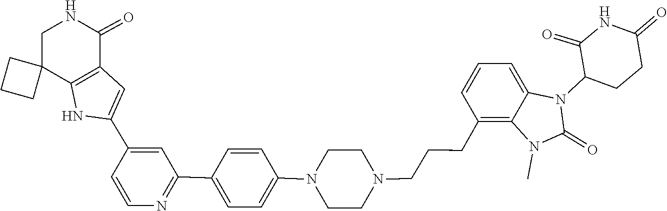 [SMILES: Cn1c(=O)n(C2CCC(=O)NC2=O)c2cccc(CCCN3CCN(c4ccc(-c5cc(-c6cc7c([nH]6)C6(CCC6)CNC7=O)ccn5)cc4)CC3)c21] |
| I-205 | 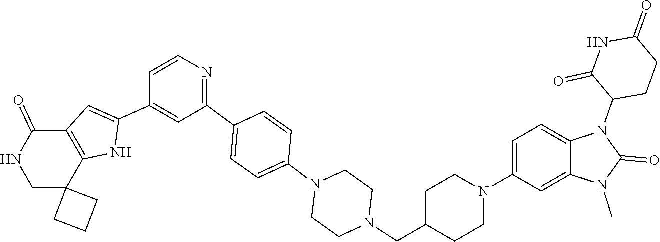 [SMILES: Cn1c(=O)n(C2CCC(=O)NC2=O)c2ccc(N3CCC(CN4CCN(c5ccc(-c6cc(-c7cc8c([nH]7)C7(CCC7)CNC8=O)ccn6)cc5)CC4)CC3)cc21] |
| I-206 | 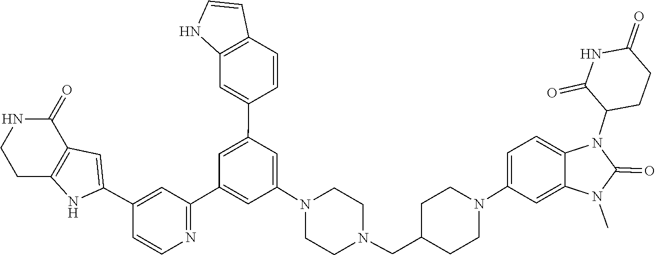 [SMILES: Cn1c(=O)n(C2CCC(=O)NC2=O)c2ccc(N3CCC(CN4CCN(c5cc(-c6ccc7cc[nH]c7c6)cc(-c6cc(-c7cc8c([nH]7)CCNC8=O)ccn6)c5)CC4)CC3)cc21] |
| I-207 | 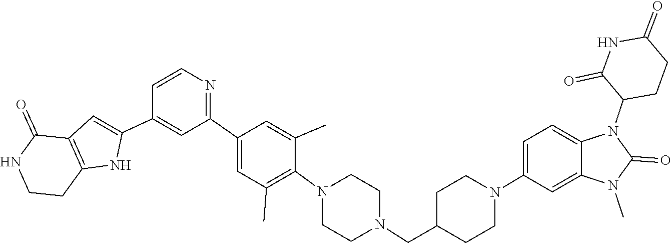 [SMILES: Cc1cc(-c2cc(-c3cc4c([nH]3)CCNC4=O)ccn2)cc(C)c1N1CCN(CC2CCN(c3ccc4c(c3)n(C)c(=O)n4C3CCC(=O)NC3=O)CC2)CC1] |
| I-208 | 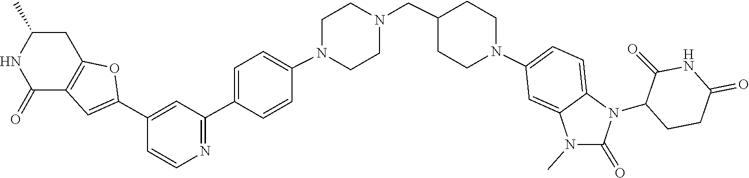 [SMILES: C[C@@H]1Cc2oc(-c3ccnc(-c4ccc(N5CCN(CC6CCN(c7ccc8c(c7)n(C)c(=O)n8C7CCC(=O)NC7=O)CC6)CC5)cc4)c3)cc2C(=O)N1] |
| I-209 | 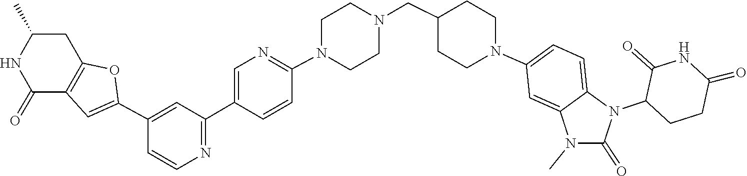 [SMILES: C[C@@H]1Cc2oc(-c3ccnc(-c4ccc(N5CCN(CC6CCN(c7ccc8c(c7)n(C)c(=O)n8C7CCC(=O)NC7=O)CC6)CC5)nc4)c3)cc2C(=O)N1] |
| I-210 | 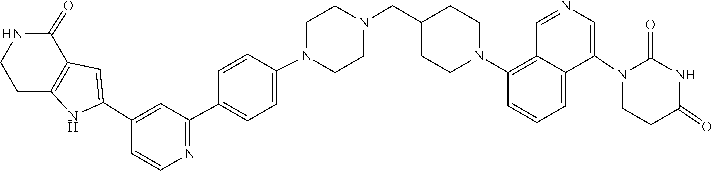 [SMILES: O=C1CCN(c2cncc3c(N4CCC(CN5CCN(c6ccc(-c7cc(-c8cc9c([nH]8)CCNC9=O)ccn7)cc6)CC5)CC4)cccc23)C(=O)N1] |
| I-211 | 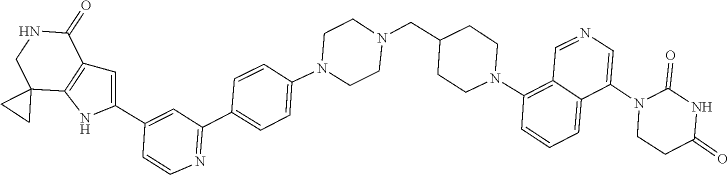 [SMILES: O=C1CCN(c2cncc3c(N4CCC(CN5CCN(c6ccc(-c7cc(-c8cc9c([nH]8)C8(CC8)CNC9=O)ccn7)cc6)CC5)CC4)cccc23)C(=O)N1] |
| I-212 | 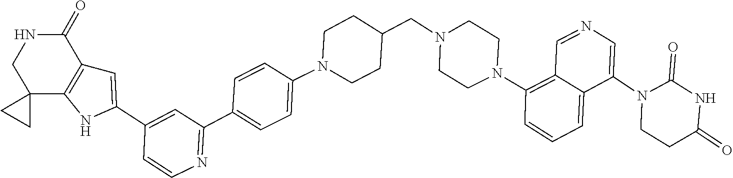 [SMILES: O=C1CCN(c2cncc3c(N4CCN(CC5CCN(c6ccc(-c7cc(-c8cc9c([nH]8)C8(CC8)CNC9=O)ccn7)cc6)CC5)CC4)cccc23)C(=O)N1] |
| I-213 | 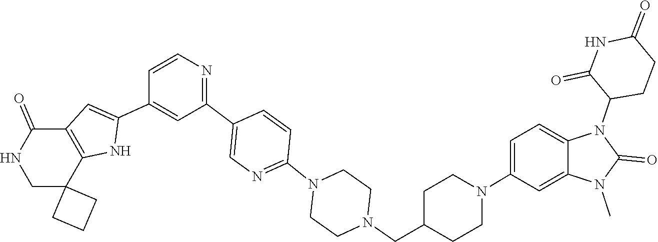 [SMILES: Cn1c(=O)n(C2CCC(=O)NC2=O)c2ccc(N3CCC(CN4CCN(c5ccc(-c6cc(-c7cc8c([nH]7)C7(CCC7)CNC8=O)ccn6)cn5)CC4)CC3)cc21] |
| I-214 | 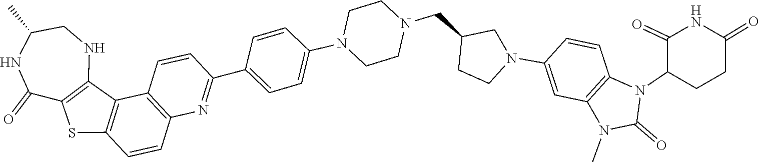 [SMILES: C[C@@H]1CNc2c(sc3ccc4nc(-c5ccc(N6CCN(C[C@@H]7CCN(c8ccc9c(c8)n(C)c(=O)n9C8CCC(=O)NC8=O)C7)CC6)cc5)ccc4c23)C(=O)N1] |
| I-215 | 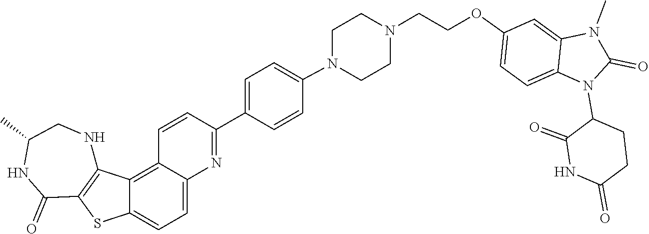 [SMILES: C[C@@H]1CNc2c(sc3ccc4nc(-c5ccc(N6CCN(CCOc7ccc8c(c7)n(C)c(=O)n8C7CCC(=O)NC7=O)CC6)cc5)ccc4c23)C(=O)N1] |
| I-216 | 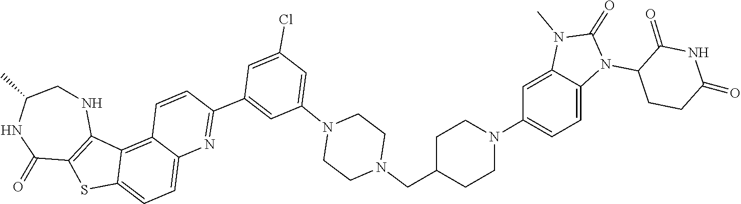 [SMILES: C[C@@H]1CNc2c(sc3ccc4nc(-c5cc(Cl)cc(N6CCN(CC7CCN(c8ccc9c(c8)n(C)c(=O)n9C8CCC(=O)NC8=O)CC7)CC6)c5)ccc4c23)C(=O)N1] |
| I-217 | 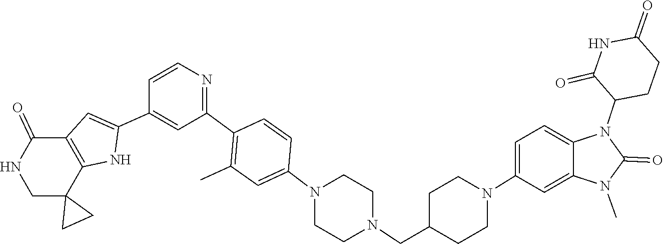 [SMILES: Cc1cc(N2CCN(CC3CCN(c4ccc5c(c4)n(C)c(=O)n5C4CCC(=O)NC4=O)CC3)CC2)ccc1-c1cc(-c2cc3c([nH]2)C2(CC2)CNC3=O)ccn1] |
| I-218 | 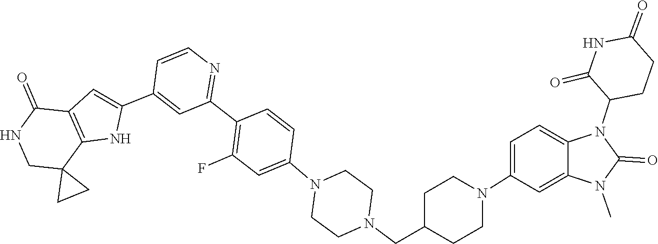 [SMILES: Cn1c(=O)n(C2CCC(=O)NC2=O)c2ccc(N3CCC(CN4CCN(c5ccc(-c6cc(-c7cc8c([nH]7)C7(CC7)CNC8=O)ccn6)c(F)c5)CC4)CC3)cc21] |
| I-219 | 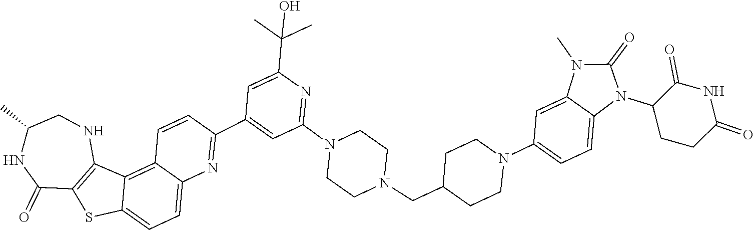 [SMILES: C[C@@H]1CNc2c(sc3ccc4nc(-c5cc(N6CCN(CC7CCN(c8ccc9c(c8)n(C)c(=O)n9C8CCC(=O)NC8=O)CC7)CC6)nc(C(C)(C)O)c5)ccc4c23)C(=O)N1] |
| I-220 | 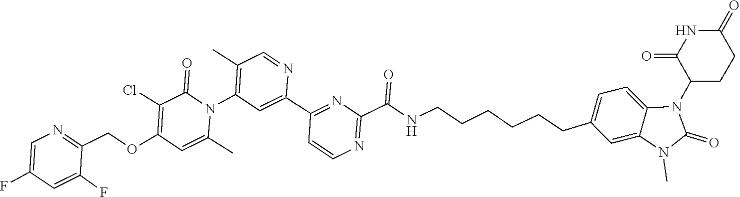 [SMILES: Cc1cnc(-c2ccnc(C(=O)NCCCCCCc3ccc4c(c3)n(C)c(=O)n4C3CCC(=O)NC3=O)n2)cc1-n1c(C)cc(OCc2ncc(F)cc2F)c(Cl)c1=O] |
| I-221 | 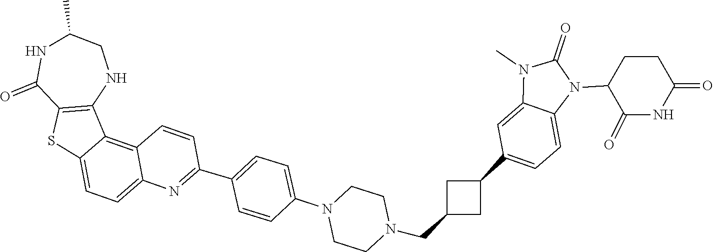 [SMILES: C[C@@H]1CNc2c(sc3ccc4nc(-c5ccc(N6CCN(C[C@H]7C[C@@H](c8ccc9c(c8)n(C)c(=O)n9C8CCC(=O)NC8=O)C7)CC6)cc5)ccc4c23)C(=O)N1] |
| I-222 | 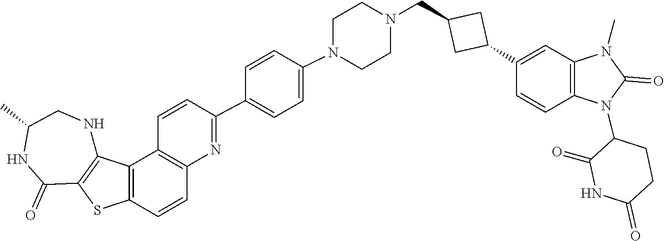 [SMILES: C[C@@H]1CNc2c(sc3ccc4nc(-c5ccc(N6CCN(C[C@H]7C[C@H](c8ccc9c(c8)n(C)c(=O)n9C8CCC(=O)NC8=O)C7)CC6)cc5)ccc4c23)C(=O)N1] |
| I-223 | 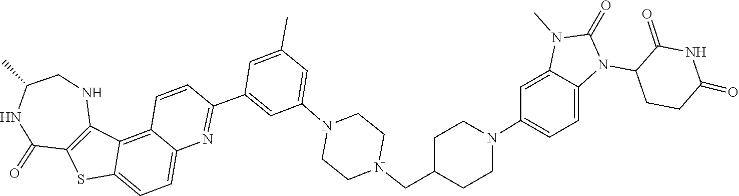 [SMILES: Cc1cc(-c2ccc3c(ccc4sc5c(c43)NC[C@@H](C)NC5=O)n2)cc(N2CCN(CC3CCN(c4ccc5c(c4)n(C)c(=O)n5C4CCC(=O)NC4=O)CC3)CC2)c1] |
| I-224 | 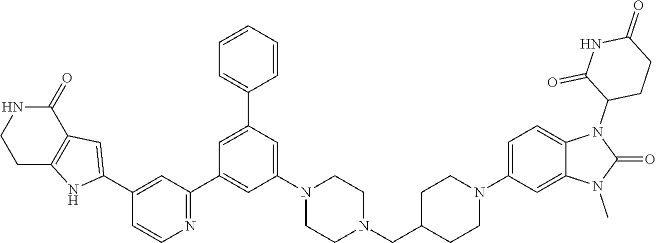 [SMILES: Cn1c(=O)n(C2CCC(=O)NC2=O)c2ccc(N3CCC(CN4CCN(c5cc(-c6ccccc6)cc(-c6cc(-c7cc8c([nH]7)CCNC8=O)ccn6)c5)CC4)CC3)cc21] |
| I-225 | 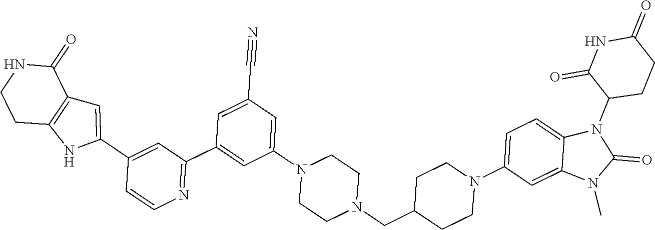 [SMILES: Cn1c(=O)n(C2CCC(=O)NC2=O)c2ccc(N3CCC(CN4CCN(c5cc(C#N)cc(-c6cc(-c7cc8c([nH]7)CCNC8=O)ccn6)c5)CC4)CC3)cc21] |
| I-226 | 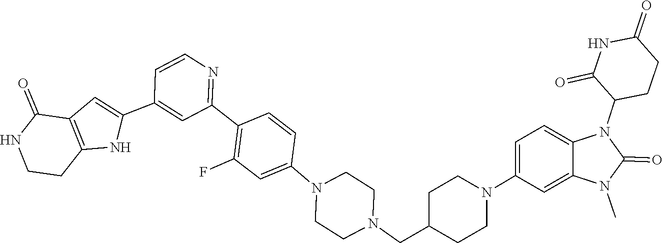 [SMILES: Cn1c(=O)n(C2CCC(=O)NC2=O)c2ccc(N3CCC(CN4CCN(c5ccc(-c6cc(-c7cc8c([nH]7)CCNC8=O)ccn6)c(F)c5)CC4)CC3)cc21] |
| I-227 | 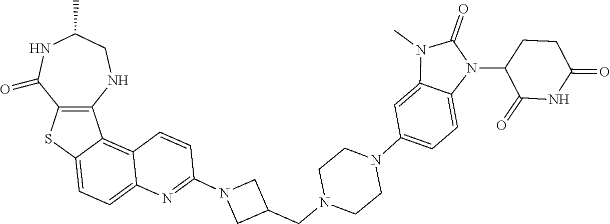 [SMILES: C[C@@H]1CNc2c(sc3ccc4nc(N5CC(CN6CCN(c7ccc8c(c7)n(C)c(=O)n8C7CCC(=O)NC7=O)CC6)C5)ccc4c23)C(=O)N1] |
| I-228 | 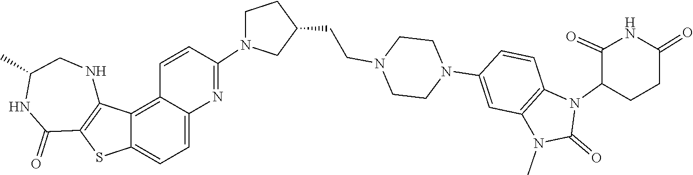 [SMILES: C[C@@H]1CNc2c(sc3ccc4nc(N5CC[C@H](CCN6CCN(c7ccc8c(c7)n(C)c(=O)n8C7CCC(=O)NC7=O)CC6)C5)ccc4c23)C(=O)N1] |
| I-229 | 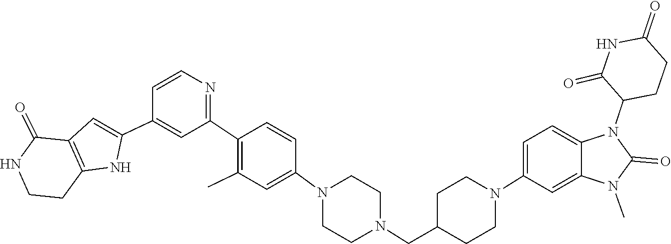 [SMILES: Cc1cc(N2CCN(CC3CCN(c4ccc5c(c4)n(C)c(=O)n5C4CCC(=O)NC4=O)CC3)CC2)ccc1-c1cc(-c2cc3c([nH]2)CCNC3=O)ccn1] |
| I-230 | 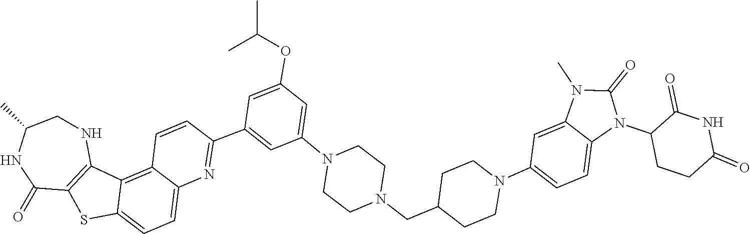 [SMILES: CC(C)Oc1cc(-c2ccc3c(ccc4sc5c(c43)NC[C@@H](C)NC5=O)n2)cc(N2CCN(CC3CCN(c4ccc5c(c4)n(C)c(=O)n5C4CCC(=O)NC4=O)CC3)CC2)c1] |
| I-231 | 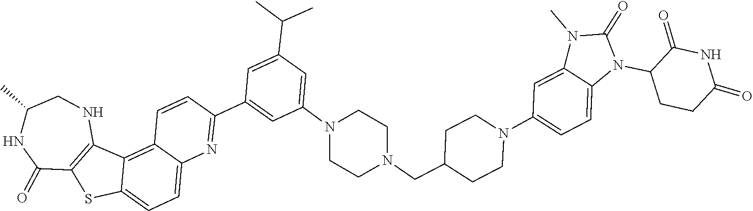 [SMILES: CC(C)c1cc(-c2ccc3c(ccc4sc5c(c43)NC[C@@H](C)NC5=O)n2)cc(N2CCN(CC3CCN(c4ccc5c(c4)n(C)c(=O)n5C4CCC(=O)NC4=O)CC3)CC2)c1] |
| I-232 | 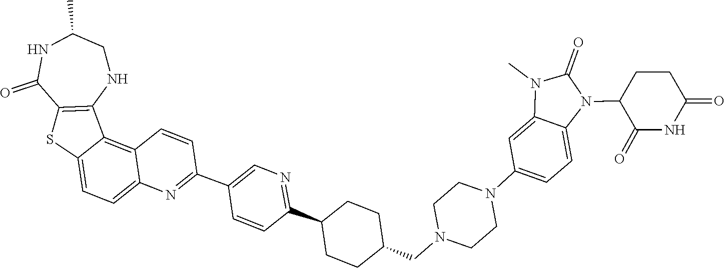 [SMILES: C[C@@H]1CNc2c(sc3ccc4nc(-c5ccc([C@H]6CC[C@H](CN7CCN(c8ccc9c(c8)n(C)c(=O)n9C8CCC(=O)NC8=O)CC7)CC6)nc5)ccc4c23)C(=O)N1] |
| I-233 | 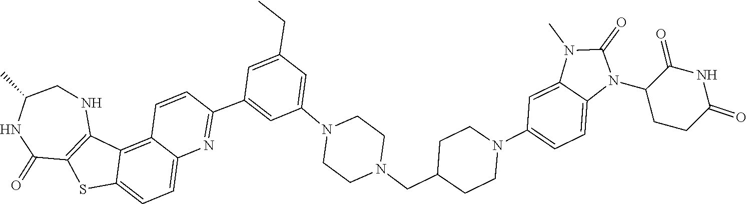 [SMILES: CCc1cc(-c2ccc3c(ccc4sc5c(c43)NC[C@@H](C)NC5=O)n2)cc(N2CCN(CC3CCN(c4ccc5c(c4)n(C)c(=O)n5C4CCC(=O)NC4=O)CC3)CC2)c1] |
| I-234 | 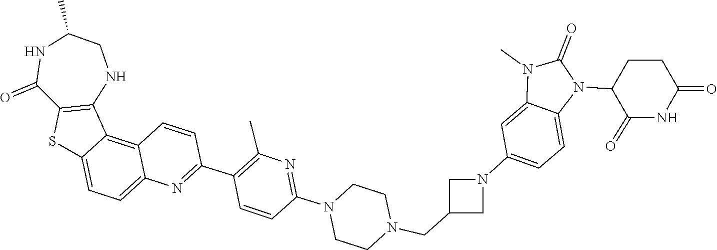 [SMILES: Cc1nc(N2CCN(CC3CN(c4ccc5c(c4)n(C)c(=O)n5C4CCC(=O)NC4=O)C3)CC2)ccc1-c1ccc2c(ccc3sc4c(c32)NC[C@@H](C)NC4=O)n1] |
| I-235 | 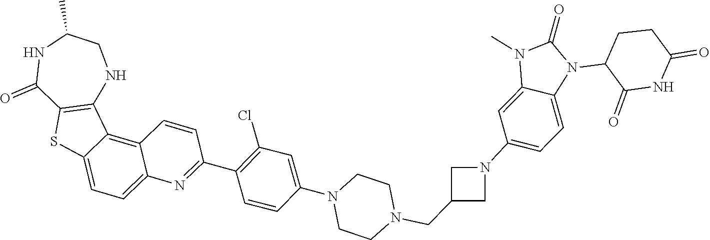 [SMILES: C[C@@H]1CNc2c(sc3ccc4nc(-c5ccc(N6CCN(CC7CN(c8ccc9c(c8)n(C)c(=O)n9C8CCC(=O)NC8=O)C7)CC6)cc5Cl)ccc4c23)C(=O)N1] |
| I-236 | 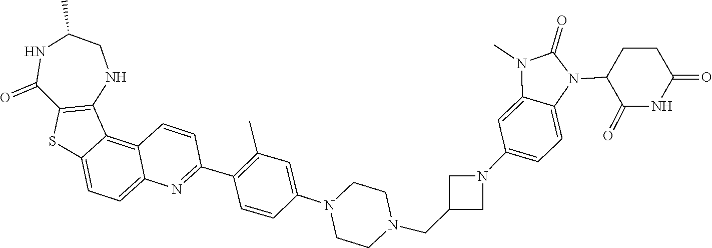 [SMILES: Cc1cc(N2CCN(CC3CN(c4ccc5c(c4)n(C)c(=O)n5C4CCC(=O)NC4=O)C3)CC2)ccc1-c1ccc2c(ccc3sc4c(c32)NC[C@@H](C)NC4=O)n1] |
| I-237 | 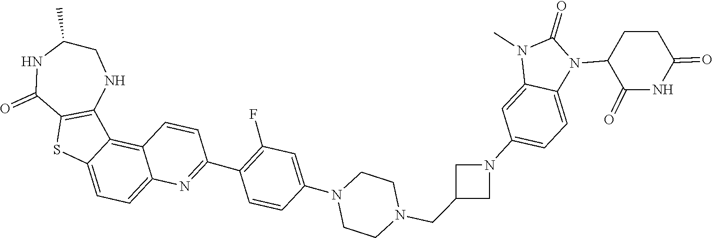 [SMILES: C[C@@H]1CNc2c(sc3ccc4nc(-c5ccc(N6CCN(CC7CN(c8ccc9c(c8)n(C)c(=O)n9C8CCC(=O)NC8=O)C7)CC6)cc5F)ccc4c23)C(=O)N1] |
| I-238 | 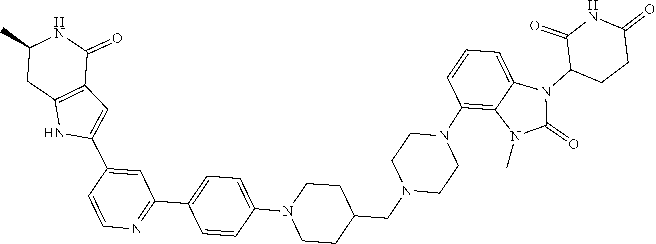 |
| I-239 | 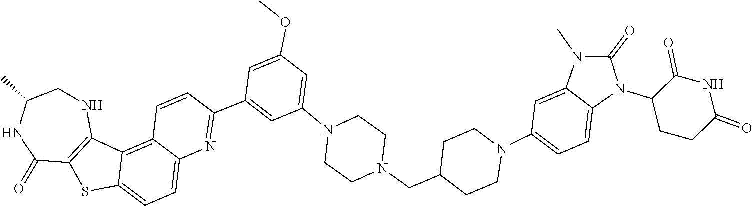 [SMILES: COc1cc(-c2ccc3c(ccc4sc5c(c43)NC[C@@H](C)NC5=O)n2)cc(N2CCN(CC3CCN(c4ccc5c(c4)n(C)c(=O)n5C4CCC(=O)NC4=O)CC3)CC2)c1] |
| I-240 | 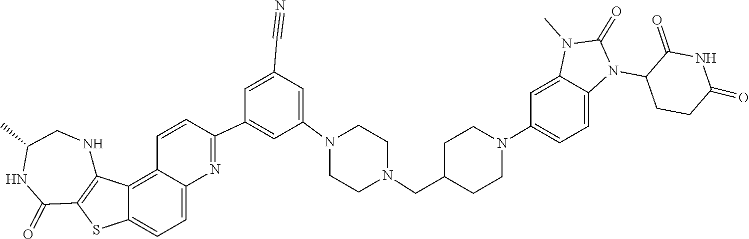 [SMILES: C[C@@H]1CNc2c(sc3ccc4nc(-c5cc(C#N)cc(N6CCN(CC7CCN(c8ccc9c(c8)n(C)c(=O)n9C8CCC(=O)NC8=O)CC7)CC6)c5)ccc4c23)C(=O)N1] |
| I-241 | 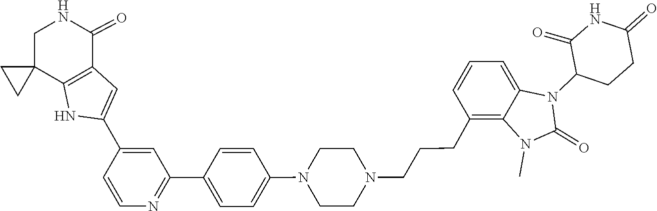 [SMILES: Cn1c(=O)n(C2CCC(=O)NC2=O)c2cccc(CCCN3CCN(c4ccc(-c5cc(-c6cc7c([nH]6)C6(CC6)CNC7=O)ccn5)cc4)CC3)c21] |
| I-242 | 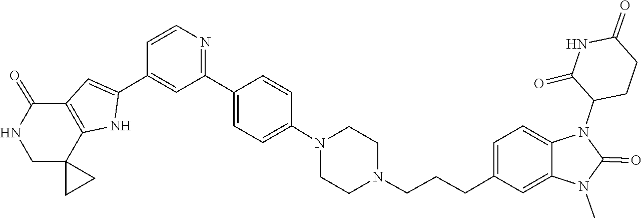 [SMILES: Cn1c(=O)n(C2CCC(=O)NC2=O)c2ccc(CCCN3CCN(c4ccc(-c5cc(-c6cc7c([nH]6)C6(CC6)CNC7=O)ccn5)cc4)CC3)cc21] |
| I-243 | 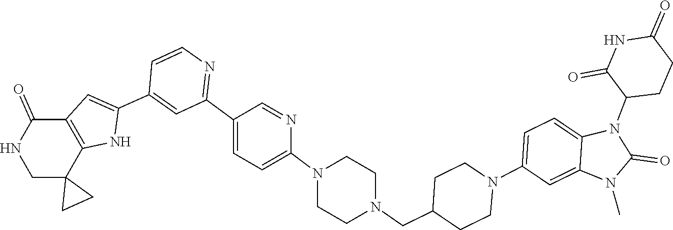 [SMILES: Cn1c(=O)n(C2CCC(=O)NC2=O)c2ccc(N3CCC(CN4CCN(c5ccc(-c6cc(-c7cc8c([nH]7)C7(CC7)CNC8=O)ccn6)cn5)CC4)CC3)cc21] |
| I-244 | 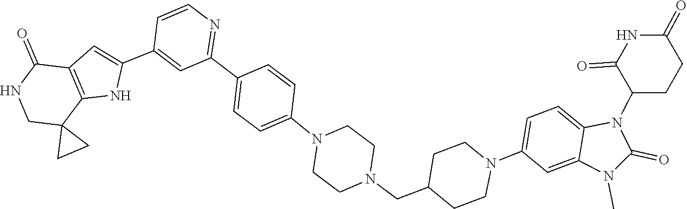 [SMILES: Cn1c(=O)n(C2CCC(=O)NC2=O)c2ccc(N3CCC(CN4CCN(c5ccc(-c6cc(-c7cc8c([nH]7)C7(CC7)CNC8=O)ccn6)cc5)CC4)CC3)cc21] |
| I-245 | 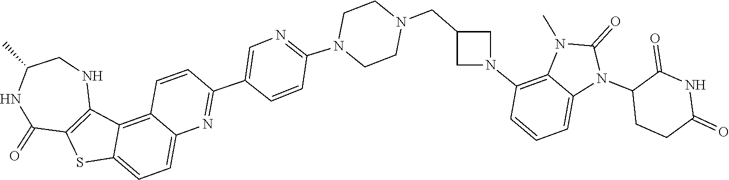 [SMILES: C[C@@H]1CNc2c(sc3ccc4nc(-c5ccc(N6CCN(CC7CN(c8cccc9c8n(C)c(=O)n9C8CCC(=O)NC8=O)C7)CC6)nc5)ccc4c23)C(=O)N1] |
| I-246 | 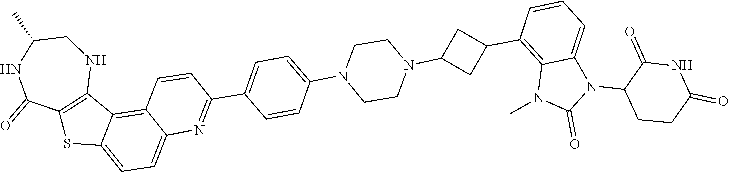 [SMILES: C[C@@H]1CNc2c(sc3ccc4nc(-c5ccc(N6CCN(C7CC(c8cccc9c8n(C)c(=O)n9C8CCC(=O)NC8=O)C7)CC6)cc5)ccc4c23)C(=O)N1] |
| I-247 | 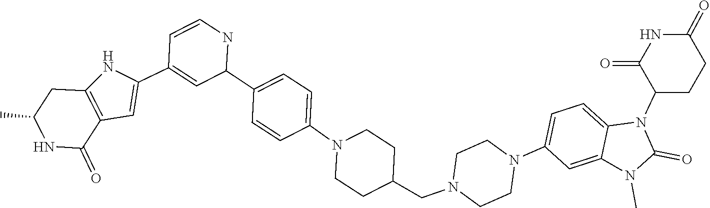 |
| I-248 | 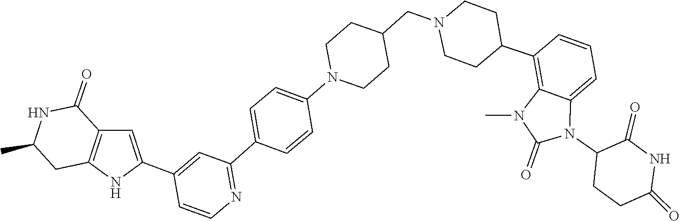 [SMILES: C[C@@H]1Cc2[nH]c(-c3ccnc(-c4ccc(N5CCC(CN6CCC(c7cccc8c7n(C)c(=O)n8C7CCC(=O)NC7=O)CC6)CC5)cc4)c3)cc2C(=O)N1] |
| I-249 | 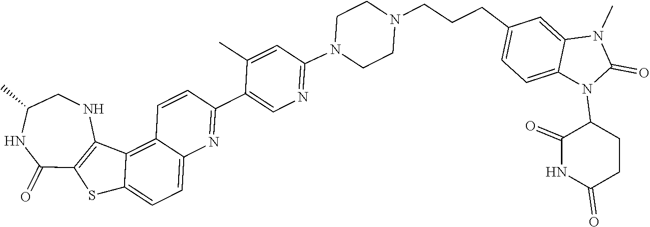 [SMILES: Cc1cc(N2CCN(CCCc3ccc4c(c3)n(C)c(=O)n4C3CCC(=O)NC3=O)CC2)ncc1-c1ccc2c(ccc3sc4c(c32)NC[C@@H](C)NC4=O)n1] |
| I-250 | 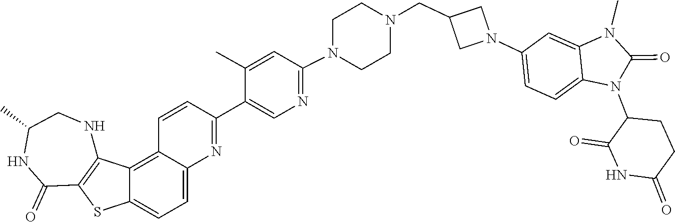 [SMILES: Cc1cc(N2CCN(CC3CN(c4ccc5c(c4)n(C)c(=O)n5C4CCC(=O)NC4=O)C3)CC2)ncc1-c1ccc2c(ccc3sc4c(c32)NC[C@@H](C)NC4=O)n1] |
| I-251 | 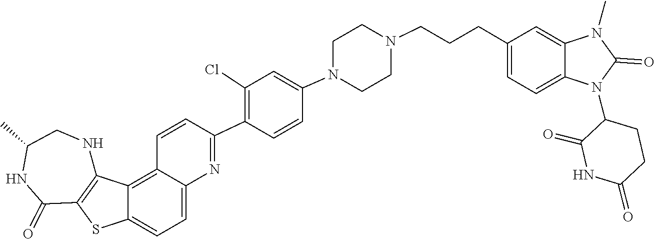 [SMILES: C[C@@H]1CNc2c(sc3ccc4nc(-c5ccc(N6CCN(CCCc7ccc8c(c7)n(C)c(=O)n8C7CCC(=O)NC7=O)CC6)cc5Cl)ccc4c23)C(=O)N1] |
| I-252 | 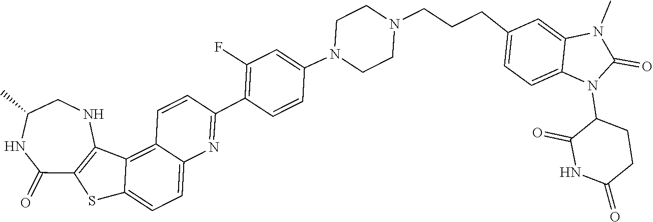 [SMILES: C[C@@H]1CNc2c(sc3ccc4nc(-c5ccc(N6CCN(CCCc7ccc8c(c7)n(C)c(=O)n8C7CCC(=O)NC7=O)CC6)cc5F)ccc4c23)C(=O)N1] |
| I-253 | 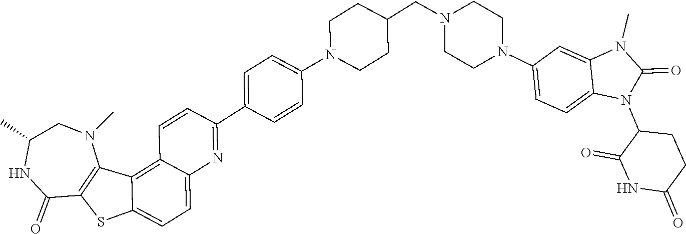 [SMILES: C[C@@H]1CN(C)c2c(sc3ccc4nc(-c5ccc(N6CCC(CN7CCN(c8ccc9c(c8)n(C)c(=O)n9C8CCC(=O)NC8=O)CC7)CC6)cc5)ccc4c23)C(=O)N1] |
| I-254 | 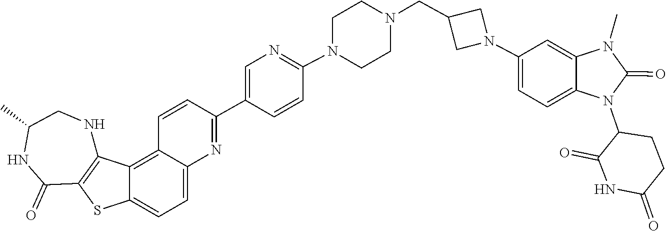 [SMILES: C[C@@H]1CNc2c(sc3ccc4nc(-c5ccc(N6CCN(CC7CN(c8ccc9c(c8)n(C)c(=O)n9C8CCC(=O)NC8=O)C7)CC6)nc5)ccc4c23)C(=O)N1] |
| I-255 | 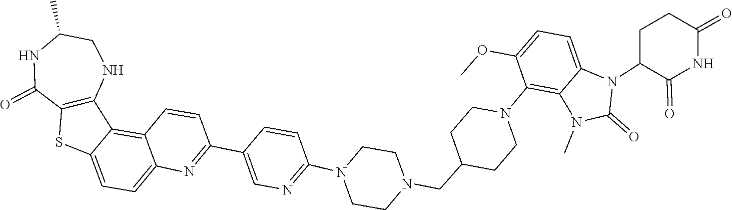 [SMILES: COc1ccc2c(c1N1CCC(CN3CCN(c4ccc(-c5ccc6c(ccc7sc8c(c76)NC[C@@H](C)NC8=O)n5)cn4)CC3)CC1)n(C)c(=O)n2C1CCC(=O)NC1=O] |
| I-256 | 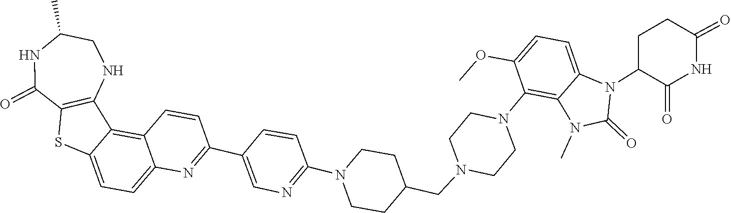 [SMILES: COc1ccc2c(c1N1CCN(CC3CCN(c4ccc(-c5ccc6c(ccc7sc8c(c76)NC[C@@H](C)NC8=O)n5)cn4)CC3)CC1)n(C)c(=O)n2C1CCC(=O)NC1=O] |
| I-257 | 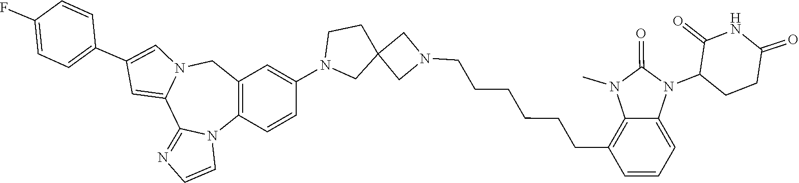 [SMILES: Cn1c(=O)n(C2CCC(=O)NC2=O)c2cccc(CCCCCCN3CC4(CCN(c5ccc6c(c5)Cn5cc(-c7ccc(F)cc7)cc5-c5nccn5-6)C4)C3)c21] |
| I-258 | 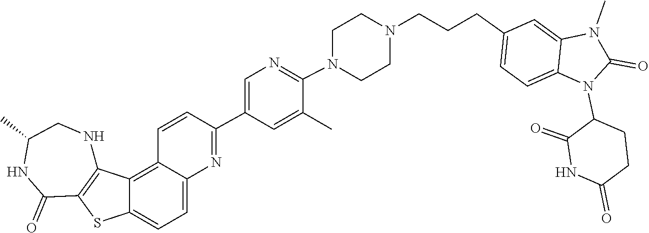 [SMILES: Cc1cc(-c2ccc3c(ccc4sc5c(c43)NC[C@@H](C)NC5=O)n2)cnc1N1CCN(CCCc2ccc3c(c2)n(C)c(=O)n3C2CCC(=O)NC2=O)CC1] |
| I-259 | 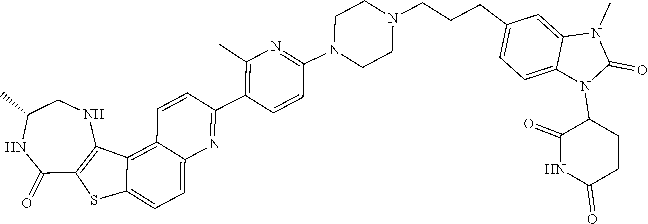 [SMILES: Cc1nc(N2CCN(CCCc3ccc4c(c3)n(C)c(=O)n4C3CCC(=O)NC3=O)CC2)ccc1-c1ccc2c(ccc3sc4c(c32)NC[C@@H](C)NC4=O)n1] |
| I-260 | 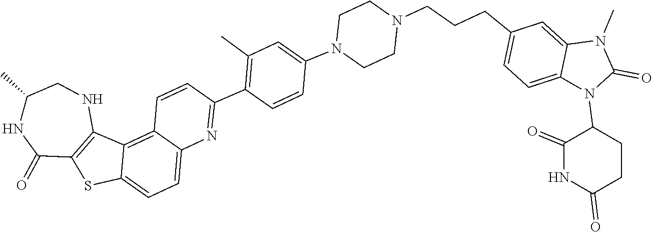 [SMILES: Cc1cc(N2CCN(CCCc3ccc4c(c3)n(C)c(=O)n4C3CCC(=O)NC3=O)CC2)ccc1-c1ccc2c(ccc3sc4c(c32)NC[C@@H](C)NC4=O)n1] |
| I-261 | 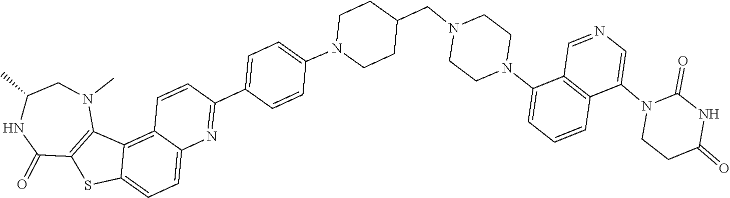 [SMILES: C[C@@H]1CN(C)c2c(sc3ccc4nc(-c5ccc(N6CCC(CN7CCN(c8cccc9c(N%10CCC(=O)NC%10=O)cncc89)CC7)CC6)cc5)ccc4c23)C(=O)N1] |
| I-262 | 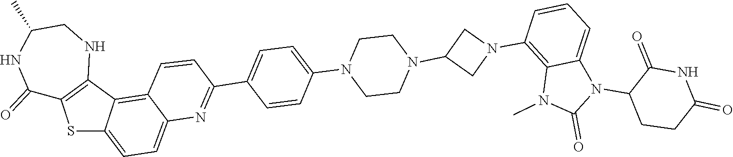 [SMILES: C[C@@H]1CNc2c(sc3ccc4nc(-c5ccc(N6CCN(C7CN(c8cccc9c8n(C)c(=O)n9C8CCC(=O)NC8=O)C7)CC6)cc5)ccc4c23)C(=O)N1] |
| I-263 | 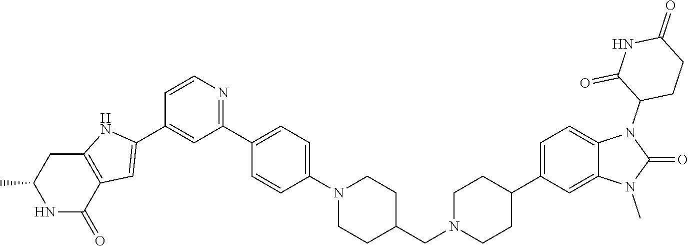 [SMILES: C[C@@H]1Cc2[nH]c(-c3ccnc(-c4ccc(N5CCC(CN6CCC(c7ccc8c(c7)n(C)c(=O)n8C7CCC(=O)NC7=O)CC6)CC5)cc4)c3)cc2C(=O)N1] |
| I-264 | 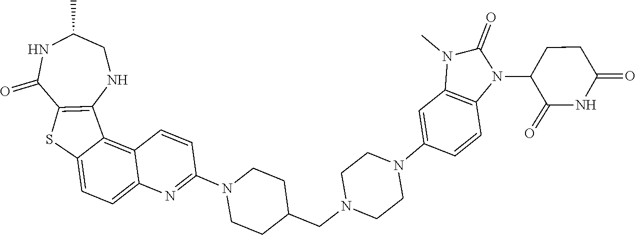 [SMILES: C[C@@H]1CNc2c(sc3ccc4nc(N5CCC(CN6CCN(c7ccc8c(c7)n(C)c(=O)n8C7CCC(=O)NC7=O)CC6)CC5)ccc4c23)C(=O)N1] |
| I-265 | 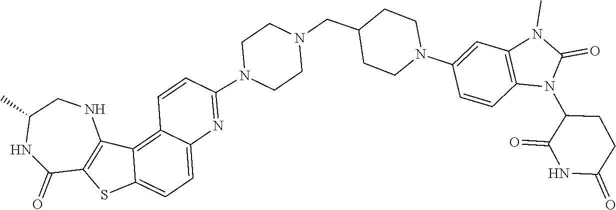 [SMILES: C[C@@H]1CNc2c(sc3ccc4nc(N5CCN(CC6CCN(c7ccc8c(c7)n(C)c(=O)n8C7CCC(=O)NC7=O)CC6)CC5)ccc4c23)C(=O)N1] |
| I-266 | 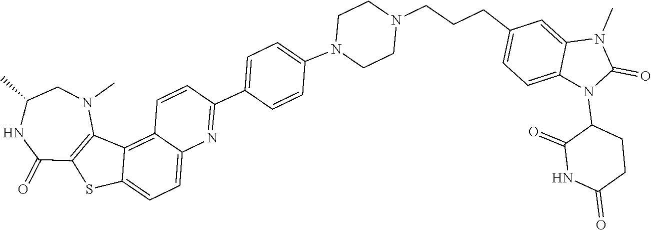 [SMILES: C[C@@H]1CN(C)c2c(sc3ccc4nc(-c5ccc(N6CCN(CCCc7ccc8c(c7)n(C)c(=O)n8C7CCC(=O)NC7=O)CC6)cc5)ccc4c23)C(=O)N1] |
| I-267 | 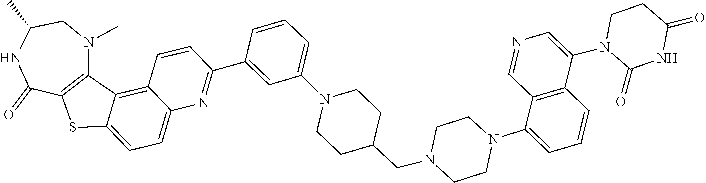 [SMILES: C[C@@H]1CN(C)c2c(sc3ccc4nc(-c5cccc(N6CCC(CN7CCN(c8cccc9c(N%10CCC(=O)NC%10=O)cncc89)CC7)CC6)c5)ccc4c23)C(=O)N1] |
| I-268 | 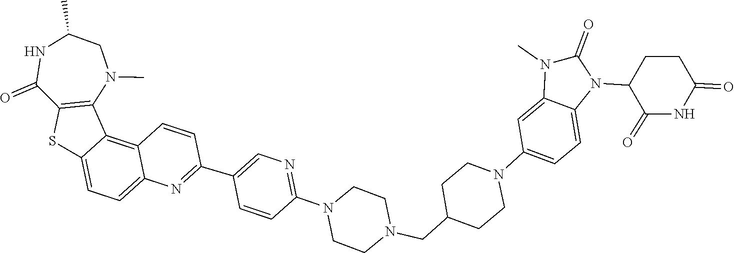 [SMILES: C[C@@H]1CN(C)c2c(sc3ccc4nc(-c5ccc(N6CCN(CC7CCN(c8ccc9c(c8)n(C)c(=O)n9C8CCC(=O)NC8=O)CC7)CC6)nc5)ccc4c23)C(=O)N1] |
| I-269 | 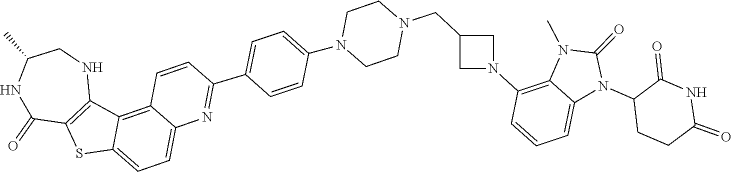 [SMILES: C[C@@H]1CNc2c(sc3ccc4nc(-c5ccc(N6CCN(CC7CN(c8cccc9c8n(C)c(=O)n9C8CCC(=O)NC8=O)C7)CC6)cc5)ccc4c23)C(=O)N1] |
| I-270 | 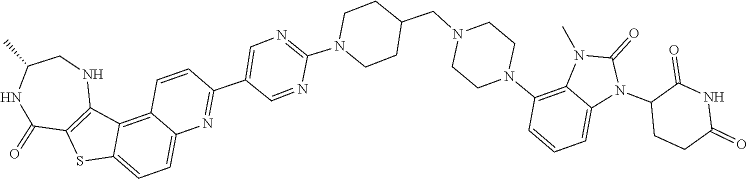 [SMILES: C[C@@H]1CNc2c(sc3ccc4nc(-c5cnc(N6CCC(CN7CCN(c8cccc9c8n(C)c(=O)n9C8CCC(=O)NC8=O)CC7)CC6)nc5)ccc4c23)C(=O)N1] |
| I-271 | 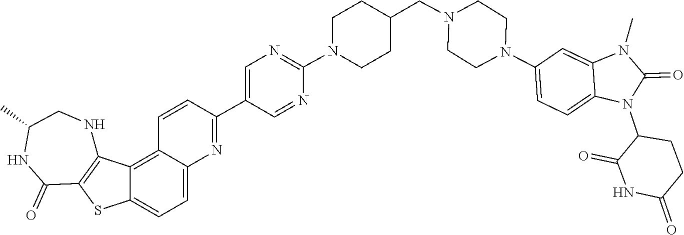 [SMILES: C[C@@H]1CNc2c(sc3ccc4nc(-c5cnc(N6CCC(CN7CCN(c8ccc9c(c8)n(C)c(=O)n9C8CCC(=O)NC8=O)CC7)CC6)nc5)ccc4c23)C(=O)N1] |
| I-272 | 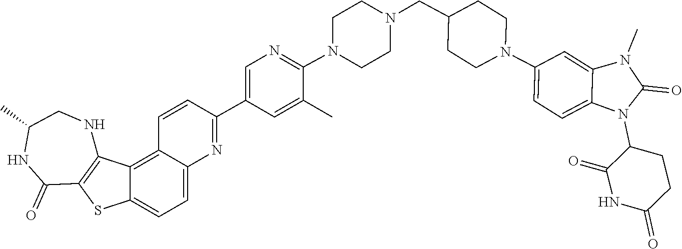 [SMILES: Cc1cc(-c2ccc3c(ccc4sc5c(c43)NC[C@@H](C)NC5=O)n2)cnc1N1CCN(CC2CCN(c3ccc4c(c3)n(C)c(=O)n4C3CCC(=O)NC3=O)CC2)CC1] |
| I-273 | 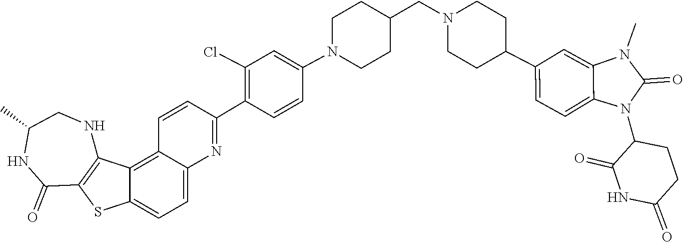 [SMILES: C[C@@H]1CNc2c(sc3ccc4nc(-c5ccc(N6CCC(CN7CCC(c8ccc9c(c8)n(C)c(=O)n9C8CCC(=O)NC8=O)CC7)CC6)cc5Cl)ccc4c23)C(=O)N1] |
| I-274 | 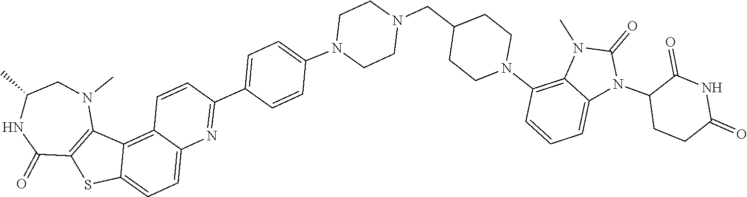 [SMILES: C[C@@H]1CN(C)c2c(sc3ccc4nc(-c5ccc(N6CCN(CC7CCN(c8cccc9c8n(C)c(=O)n9C8CCC(=O)NC8=O)CC7)CC6)cc5)ccc4c23)C(=O)N1] |
| I-275 | 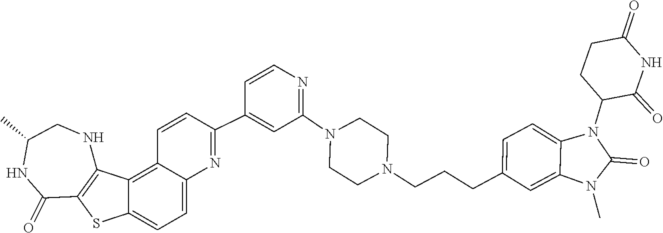 [SMILES: C[C@@H]1CNc2c(sc3ccc4nc(-c5ccnc(N6CCN(CCCc7ccc8c(c7)n(C)c(=O)n8C7CCC(=O)NC7=O)CC6)c5)ccc4c23)C(=O)N1] |
| I-276 | 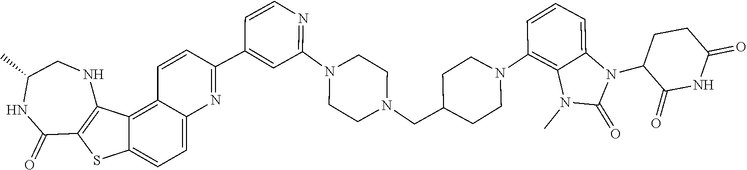 [SMILES: C[C@@H]1CNc2c(sc3ccc4nc(-c5ccnc(N6CCN(CC7CCN(c8cccc9c8n(C)c(=O)n9C8CCC(=O)NC8=O)CC7)CC6)c5)ccc4c23)C(=O)N1] |
| I-277 | 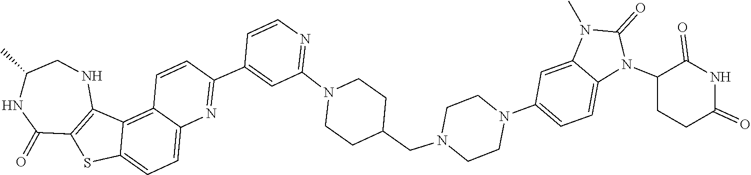 [SMILES: C[C@@H]1CNc2c(sc3ccc4nc(-c5ccnc(N6CCC(CN7CCN(c8ccc9c(c8)n(C)c(=O)n9C8CCC(=O)NC8=O)CC7)CC6)c5)ccc4c23)C(=O)N1] |
| I-278 | 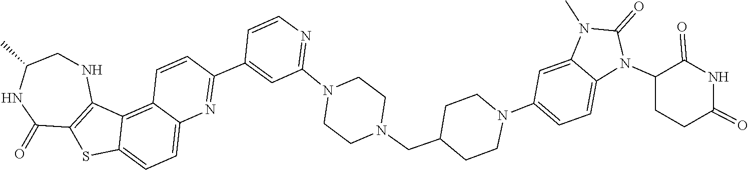 [SMILES: C[C@@H]1CNc2c(sc3ccc4nc(-c5ccnc(N6CCN(CC7CCN(c8ccc9c(c8)n(C)c(=O)n9C8CCC(=O)NC8=O)CC7)CC6)c5)ccc4c23)C(=O)N1] |
| I-279 | 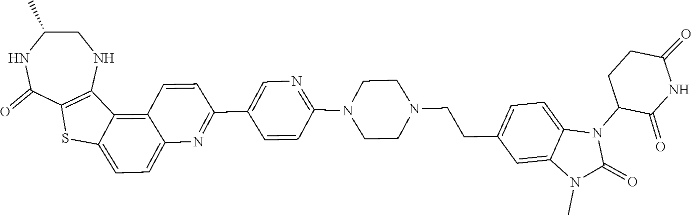 [SMILES: C[C@@H]1CNc2c(sc3ccc4nc(-c5ccc(N6CCN(CCc7ccc8c(c7)n(C)c(=O)n8C7CCC(=O)NC7=O)CC6)nc5)ccc4c23)C(=O)N1] |
| I-280 | 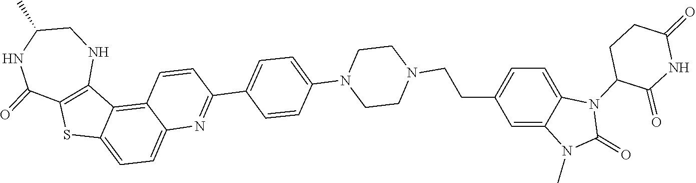 [SMILES: C[C@@H]1CNc2c(sc3ccc4nc(-c5ccc(N6CCN(CCc7ccc8c(c7)n(C)c(=O)n8C7CCC(=O)NC7=O)CC6)cc5)ccc4c23)C(=O)N1] |
| I-281 | 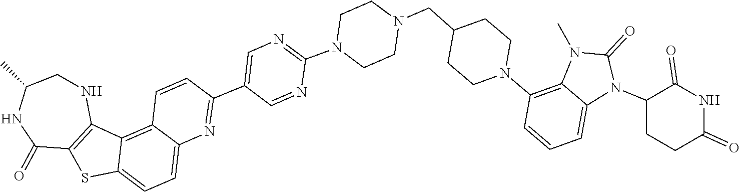 [SMILES: C[C@@H]1CNc2c(sc3ccc4nc(-c5cnc(N6CCN(CC7CCN(c8cccc9c8n(C)c(=O)n9C8CCC(=O)NC8=O)CC7)CC6)nc5)ccc4c23)C(=O)N1] |
| I-282 | 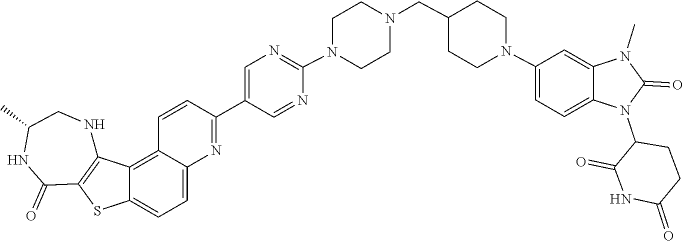 [SMILES: C[C@@H]1CNc2c(sc3ccc4nc(-c5cnc(N6CCN(CC7CCN(c8ccc9c(c8)n(C)c(=O)n9C8CCC(=O)NC8=O)CC7)CC6)nc5)ccc4c23)C(=O)N1] |
| I-283 | 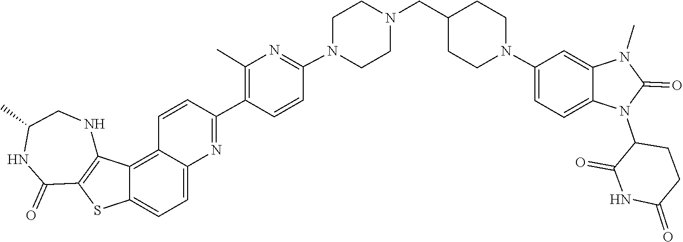 [SMILES: Cc1nc(N2CCN(CC3CCN(c4ccc5c(c4)n(C)c(=O)n5C4CCC(=O)NC4=O)CC3)CC2)ccc1-c1ccc2c(ccc3sc4c(c32)NC[C@@H](C)NC4=O)n1] |
| I-284 | 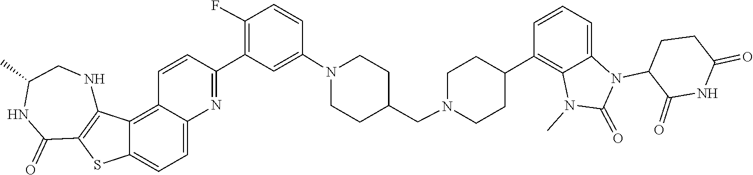 [SMILES: C[C@@H]1CNc2c(sc3ccc4nc(-c5cc(N6CCC(CN7CCC(c8cccc9c8n(C)c(=O)n9C8CCC(=O)NC8=O)CC7)CC6)ccc5F)ccc4c23)C(=O)N1] |
| I-285 | 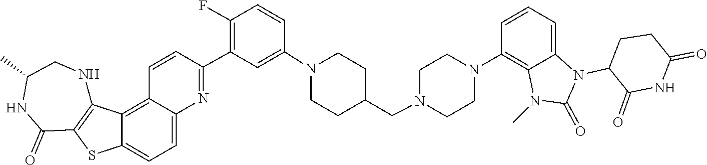 [SMILES: C[C@@H]1CNc2c(sc3ccc4nc(-c5cc(N6CCC(CN7CCN(c8cccc9c8n(C)c(=O)n9C8CCC(=O)NC8=O)CC7)CC6)ccc5F)ccc4c23)C(=O)N1] |
| I-286 | 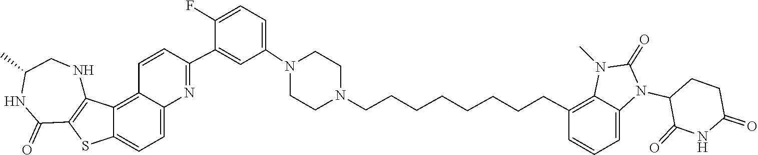 [SMILES: C[C@@H]1CNc2c(sc3ccc4nc(-c5cc(N6CCN(CCCCCCCCc7cccc8c7n(C)c(=O)n8C7CCC(=O)NC7=O)CC6)ccc5F)ccc4c23)C(=O)N1] |
| I-287 | 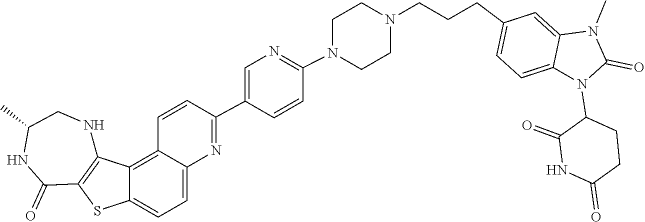 [SMILES: C[C@@H]1CNc2c(sc3ccc4nc(-c5ccc(N6CCN(CCCc7ccc8c(c7)n(C)c(=O)n8C7CCC(=O)NC7=O)CC6)nc5)ccc4c23)C(=O)N1] |
| I-288 | 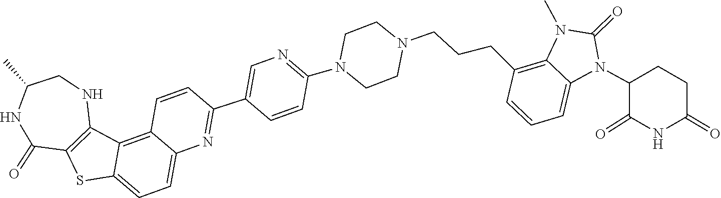 [SMILES: C[C@@H]1CNc2c(sc3ccc4nc(-c5ccc(N6CCN(CCCc7cccc8c7n(C)c(=O)n8C7CCC(=O)NC7=O)CC6)nc5)ccc4c23)C(=O)N1] |
| I-289 | 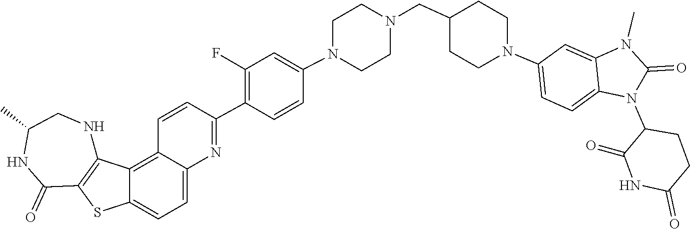 [SMILES: C[C@@H]1CNc2c(sc3ccc4nc(-c5ccc(N6CCN(CC7CCN(c8ccc9c(c8)n(C)c(=O)n9C8CCC(=O)NC8=O)CC7)CC6)cc5F)ccc4c23)C(=O)N1] |
| I-290 | 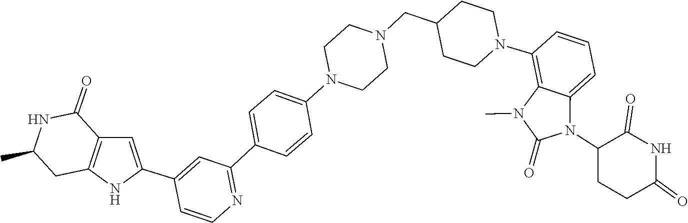 [SMILES: C[C@@H]1Cc2[nH]c(-c3ccnc(-c4ccc(N5CCN(CC6CCN(c7cccc8c7n(C)c(=O)n8C7CCC(=O)NC7=O)CC6)CC5)cc4)c3)cc2C(=O)N1] |
| I-291 | 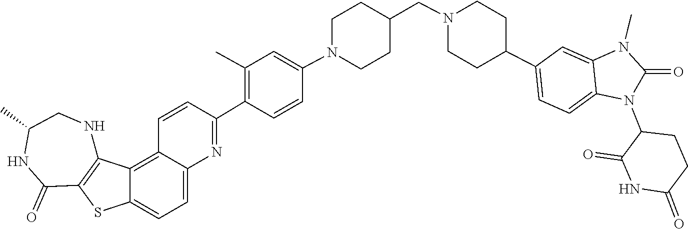 [SMILES: Cc1cc(N2CCC(CN3CCC(c4ccc5c(c4)n(C)c(=O)n5C4CCC(=O)NC4=O)CC3)CC2)ccc1-c1ccc2c(ccc3sc4c(c32)NC[C@@H](C)NC4=O)n1] |
| I-292 | 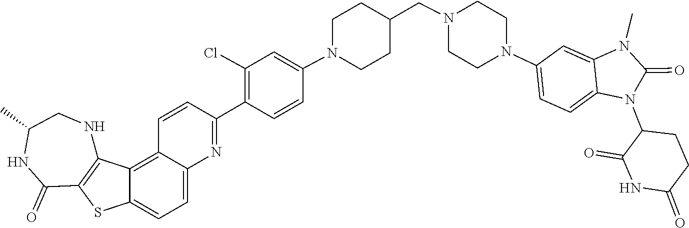 [SMILES: C[C@@H]1CNc2c(sc3ccc4nc(-c5ccc(N6CCC(CN7CCN(c8ccc9c(c8)n(C)c(=O)n9C8CCC(=O)NC8=O)CC7)CC6)cc5Cl)ccc4c23)C(=O)N1] |
| I-293 | 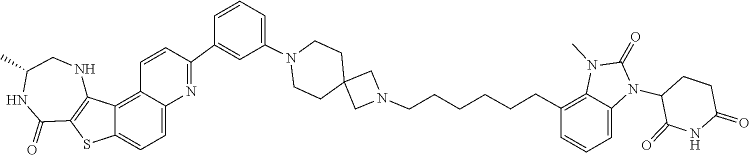 [SMILES: C[C@@H]1CNc2c(sc3ccc4nc(-c5cccc(N6CCC7(CC6)CN(CCCCCCc6cccc8c6n(C)c(=O)n8C6CCC(=O)NC6=O)C7)c5)ccc4c23)C(=O)N1] |
| I-294 | 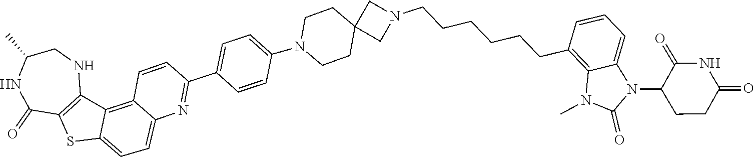 [SMILES: C[C@@H]1CNc2c(sc3ccc4nc(-c5ccc(N6CCC7(CC6)CN(CCCCCCc6cccc8c6n(C)c(=O)n8C6CCC(=O)NC6=O)C7)cc5)ccc4c23)C(=O)N1] |
| I-295 | 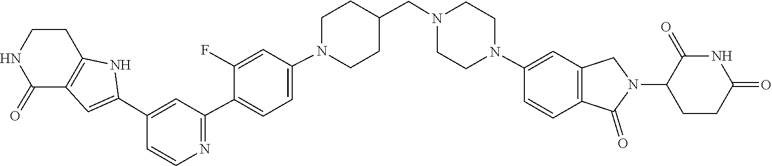 [SMILES: O=C1CCC(N2Cc3cc(N4CCN(CC5CCN(c6ccc(-c7cc(-c8cc9c([nH]8)CCNC9=O)ccn7)c(F)c6)CC5)CC4)ccc3C2=O)C(=O)N1] |
| I-296 | 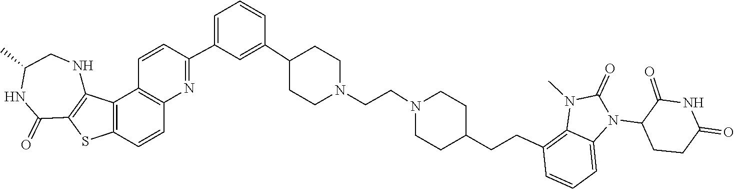 [SMILES: C[C@@H]1CNc2c(sc3ccc4nc(-c5cccc(C6CCN(CCN7CCC(CCc8cccc9c8n(C)c(=O)n9C8CCC(=O)NC8=O)CC7)CC6)c5)ccc4c23)C(=O)N1] |
| I-297 | 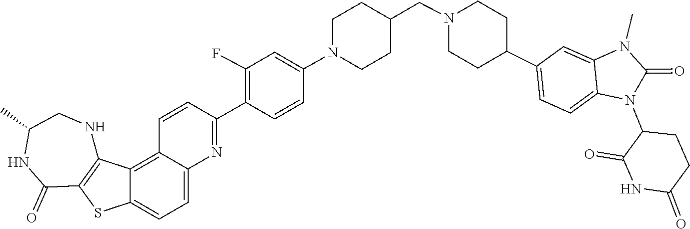 [SMILES: C[C@@H]1CNc2c(sc3ccc4nc(-c5ccc(N6CCC(CN7CCC(c8ccc9c(c8)n(C)c(=O)n9C8CCC(=O)NC8=O)CC7)CC6)cc5F)ccc4c23)C(=O)N1] |
| I-298 | 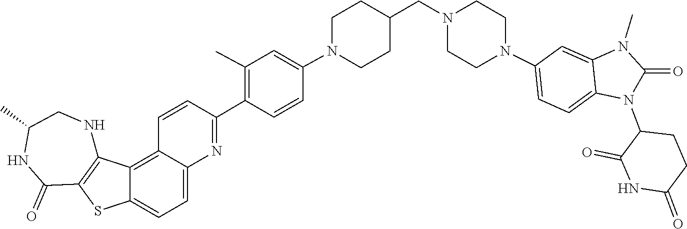 [SMILES: Cc1cc(N2CCC(CN3CCN(c4ccc5c(c4)n(C)c(=O)n5C4CCC(=O)NC4=O)CC3)CC2)ccc1-c1ccc2c(ccc3sc4c(c32)NC[C@@H](C)NC4=O)n1] |
| I-299 | 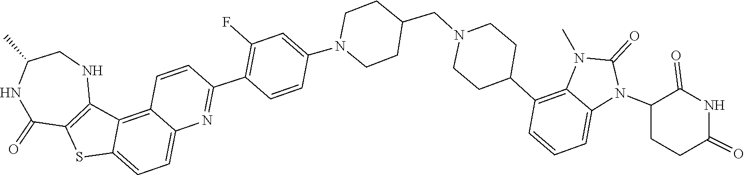 [SMILES: C[C@@H]1CNc2c(sc3ccc4nc(-c5ccc(N6CCC(CN7CCC(c8cccc9c8n(C)c(=O)n9C8CCC(=O)NC8=O)CC7)CC6)cc5F)ccc4c23)C(=O)N1] |
| I-300 | 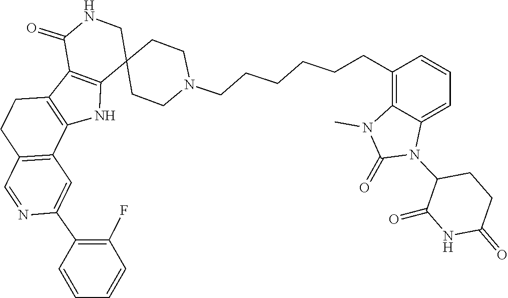 [SMILES: Cn1c(=O)n(C2CCC(=O)NC2=O)c2cccc(CCCCCCN3CCC4(CC3)CNC(=O)c3c4[nH]c4c3CCc3cnc(-c5ccccc5F)cc3-4)c21] |
| I-301 | 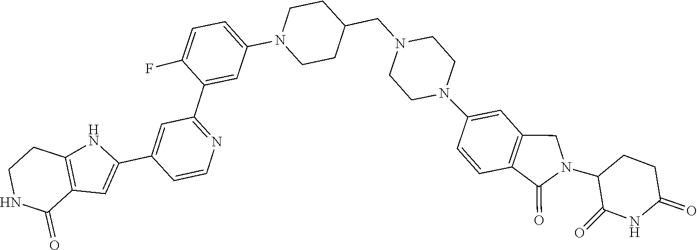 [SMILES: O=C1CCC(N2Cc3cc(N4CCN(CC5CCN(c6ccc(F)c(-c7cc(-c8cc9c([nH]8)CCNC9=O)ccn7)c6)CC5)CC4)ccc3C2=O)C(=O)N1] |
| I-302 | 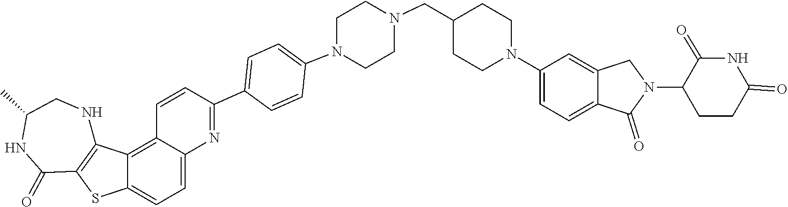 [SMILES: C[C@@H]1CNc2c(sc3ccc4nc(-c5ccc(N6CCN(CC7CCN(c8ccc9c(c8)CN(C8CCC(=O)NC8=O)C9=O)CC7)CC6)cc5)ccc4c23)C(=O)N1] |
| I-303 | 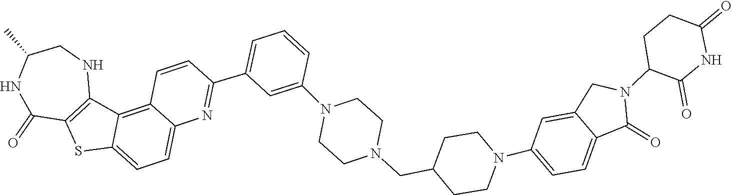 [SMILES: C[C@@H]1CNc2c(sc3ccc4nc(-c5cccc(N6CCN(CC7CCN(c8ccc9c(c8)CN(C8CCC(=O)NC8=O)C9=O)CC7)CC6)c5)ccc4c23)C(=O)N1] |
| I-304 | 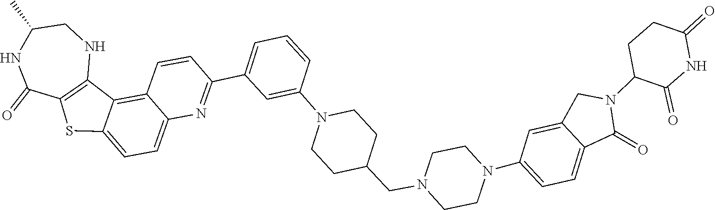 [SMILES: C[C@@H]1CNc2c(sc3ccc4nc(-c5cccc(N6CCC(CN7CCN(c8ccc9c(c8)CN(C8CCC(=O)NC8=O)C9=O)CC7)CC6)c5)ccc4c23)C(=O)N1] |
| I-305 | 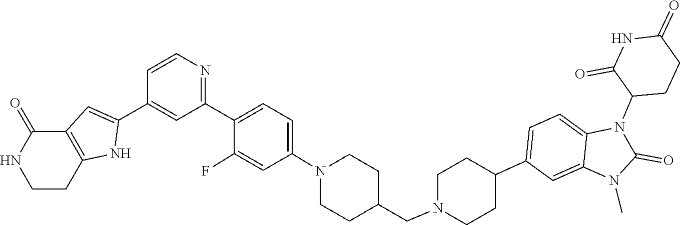 [SMILES: Cn1c(=O)n(C2CCC(=O)NC2=O)c2ccc(C3CCN(CC4CCN(c5ccc(-c6cc(-c7cc8c([nH]7)CCNC8=O)ccn6)c(F)c5)CC4)CC3)cc21] |
| I-306 | 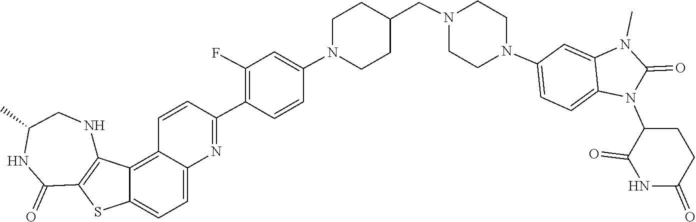 [SMILES: C[C@@H]1CNc2c(sc3ccc4nc(-c5ccc(N6CCC(CN7CCN(c8ccc9c(c8)n(C)c(=O)n9C8CCC(=O)NC8=O)CC7)CC6)cc5F)ccc4c23)C(=O)N1] |
| I-307 | 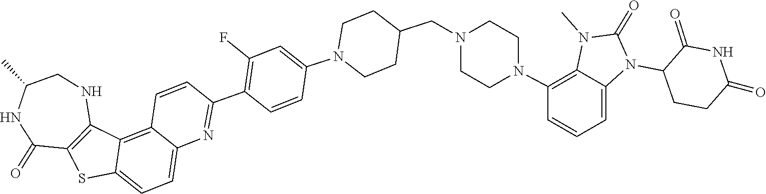 [SMILES: C[C@@H]1CNc2c(sc3ccc4nc(-c5ccc(N6CCC(CN7CCN(c8cccc9c8n(C)c(=O)n9C8CCC(=O)NC8=O)CC7)CC6)cc5F)ccc4c23)C(=O)N1] |
| I-308 | 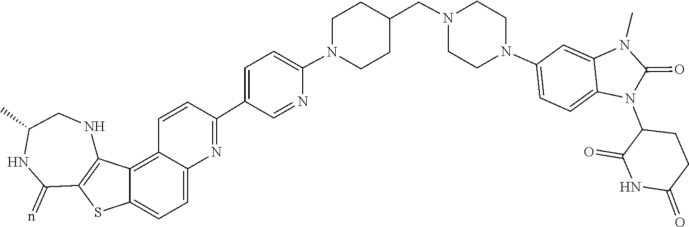 |
| I-309 | 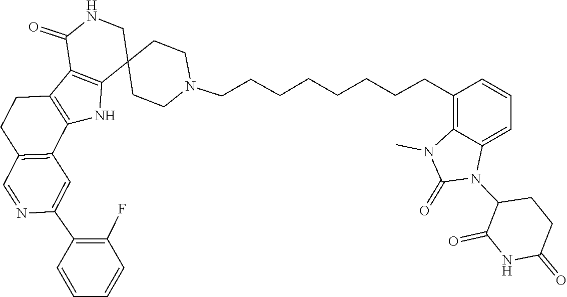 [SMILES: Cn1c(=O)n(C2CCC(=O)NC2=O)c2cccc(CCCCCCCCN3CCC4(CC3)CNC(=O)c3c4[nH]c4c3CCc3cnc(-c5ccccc5F)cc3-4)c21] |
| I-310 | 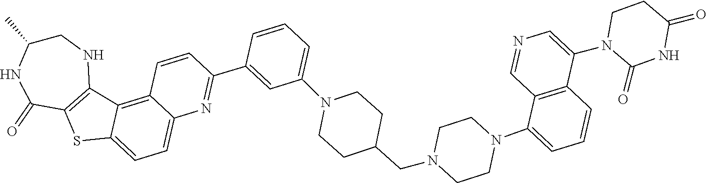 [SMILES: C[C@@H]1CNc2c(sc3ccc4nc(-c5cccc(N6CCC(CN7CCN(c8cccc9c(N%10CCC(=O)NC%10=O)cncc89)CC7)CC6)c5)ccc4c23)C(=O)N1] |
| I-311 | 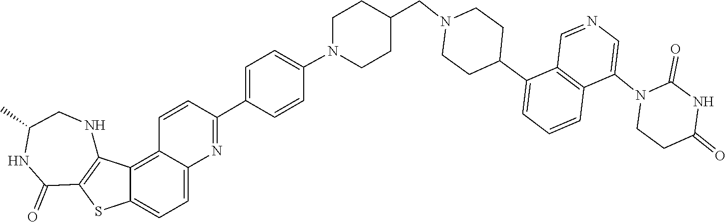 [SMILES: C[C@@H]1CNc2c(sc3ccc4nc(-c5ccc(N6CCC(CN7CCC(c8cccc9c(N%10CCC(=O)NC%10=O)cncc89)CC7)CC6)cc5)ccc4c23)C(=O)N1] |
| I-312 | 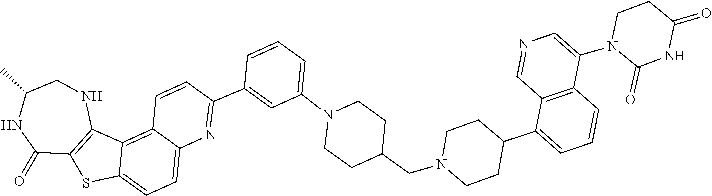 [SMILES: C[C@@H]1CNc2c(sc3ccc4nc(-c5cccc(N6CCC(CN7CCC(c8cccc9c(N%10CCC(=O)NC%10=O)cncc89)CC7)CC6)c5)ccc4c23)C(=O)N1] |
| I-313 | 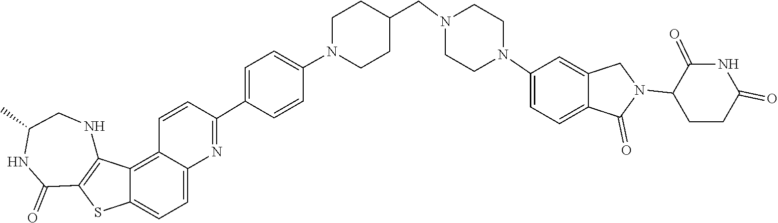 [SMILES: C[C@@H]1CNc2c(sc3ccc4nc(-c5ccc(N6CCC(CN7CCN(c8ccc9c(c8)CN(C8CCC(=O)NC8=O)C9=O)CC7)CC6)cc5)ccc4c23)C(=O)N1] |
| I-314 | 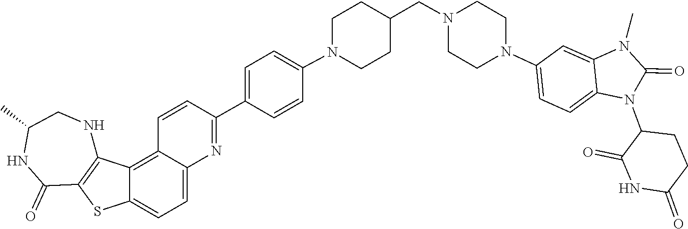 [SMILES: C[C@@H]1CNc2c(sc3ccc4nc(-c5ccc(N6CCC(CN7CCN(c8ccc9c(c8)n(C)c(=O)n9C8CCC(=O)NC8=O)CC7)CC6)cc5)ccc4c23)C(=O)N1] |
| I-315 | 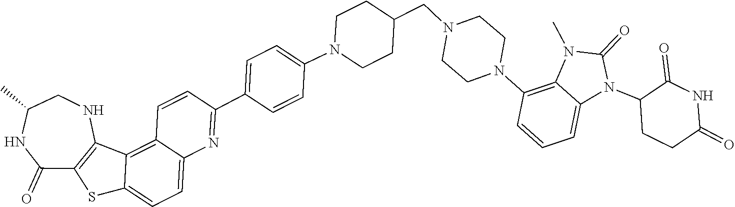 [SMILES: C[C@@H]1CNc2c(sc3ccc4nc(-c5ccc(N6CCC(CN7CCN(c8cccc9c8n(C)c(=O)n9C8CCC(=O)NC8=O)CC7)CC6)cc5)ccc4c23)C(=O)N1] |
| I-316 | 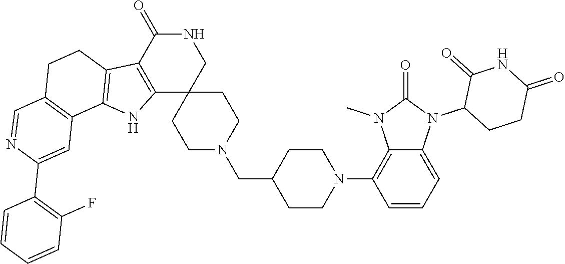 [SMILES: Cn1c(=O)n(C2CCC(=O)NC2=O)c2cccc(N3CCC(CN4CCC5(CC4)CNC(=O)c4c5[nH]c5c4CCc4cnc(-c6ccccc6F)cc4-5)CC3)c21] |
| I-317 | 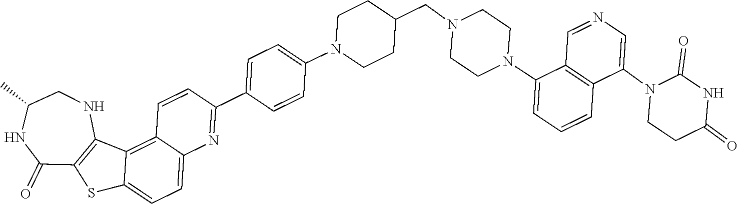 [SMILES: C[C@@H]1CNc2c(sc3ccc4nc(-c5ccc(N6CCC(CN7CCN(c8cccc9c(N%10CCC(=O)NC%10=O)cncc89)CC7)CC6)cc5)ccc4c23)C(=O)N1] |
| I-318 | 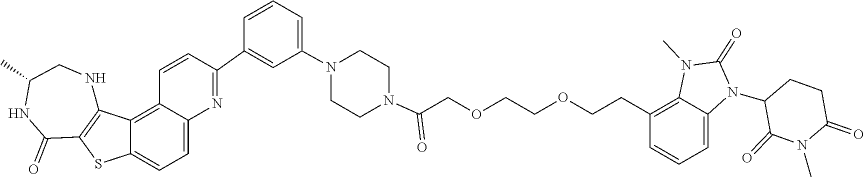 [SMILES: C[C@@H]1CNc2c(sc3ccc4nc(-c5cccc(N6CCN(C(=O)COCCOCCc7cccc8c7n(C)c(=O)n8C7CCC(=O)N(C)C7=O)CC6)c5)ccc4c23)C(=O)N1] |
| I-319 | 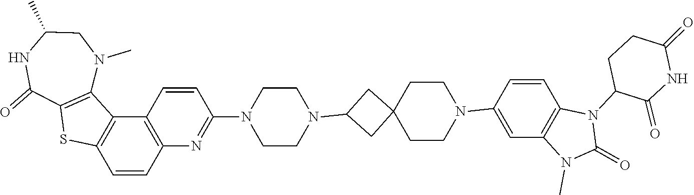 [SMILES: C[C@@H]1CN(C)c2c(sc3ccc4nc(N5CCN(C6CC7(CCN(c8ccc9c(c8)n(C)c(=O)n9C8CCC(=O)NC8=O)CC7)C6)CC5)ccc4c23)C(=O)N1] |
| I-320 | 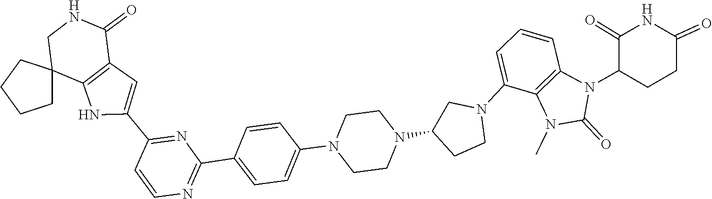 [SMILES: Cn1c(=O)n(C2CCC(=O)NC2=O)c2cccc(N3CC[C@H](N4CCN(c5ccc(-c6nccc(-c7cc8c([nH]7)C7(CCCC7)CNC8=O)n6)cc5)CC4)C3)c21] |
| I-321 | 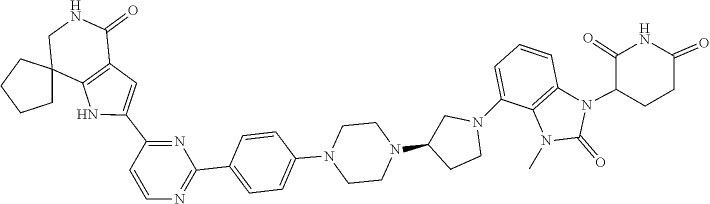 [SMILES: Cn1c(=O)n(C2CCC(=O)NC2=O)c2cccc(N3CC[C@@H](N4CCN(c5ccc(-c6nccc(-c7cc8c([nH]7)C7(CCCC7)CNC8=O)n6)cc5)CC4)C3)c21] |
| I-322 | 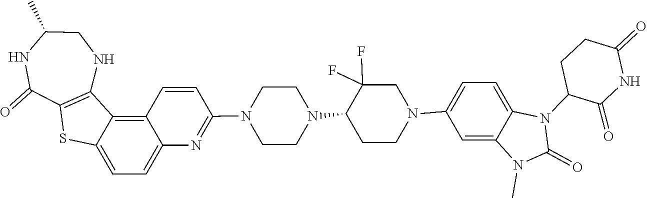 [SMILES: C[C@@H]1CNc2c(sc3ccc4nc(N5CCN([C@H]6CCN(c7ccc8c(c7)n(C)c(=O)n8C7CCC(=O)NC7=O)CC6(F)F)CC5)ccc4c23)C(=O)N1] |
| I-323 | 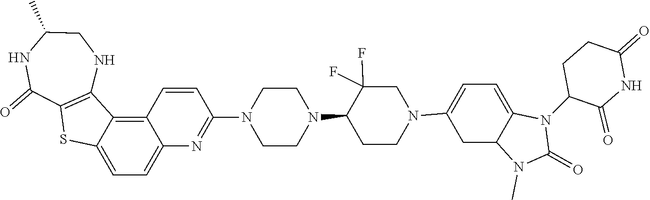 [SMILES: C[C@@H]1CNc2c(sc3ccc4nc(N5CCN([C@@H]6CCN(C7=CC=C8C(C7)N(C)C(=O)N8C7CCC(=O)NC7=O)CC6(F)F)CC5)ccc4c23)C(=O)N1] |
| I-324 | 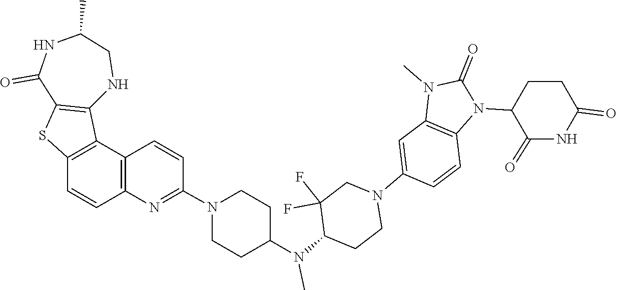 [SMILES: C[C@@H]1CNc2c(sc3ccc4nc(N5CCC(N(C)[C@H]6CCN(c7ccc8c(c7)n(C)c(=O)n8C7CCC(=O)NC7=O)CC6(F)F)CC5)ccc4c23)C(=O)N1] |
| I-325 | 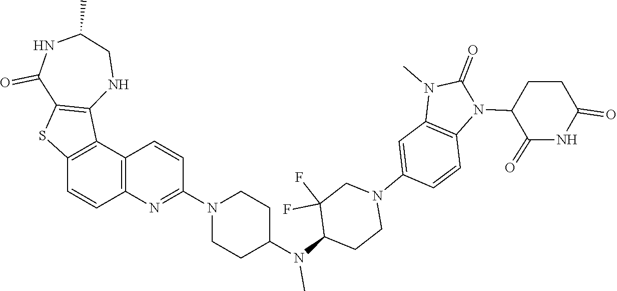 [SMILES: C[C@@H]1CNc2c(sc3ccc4nc(N5CCC(N(C)[C@@H]6CCN(c7ccc8c(c7)n(C)c(=O)n8C7CCC(=O)NC7=O)CC6(F)F)CC5)ccc4c23)C(=O)N1] |
| I-326 | 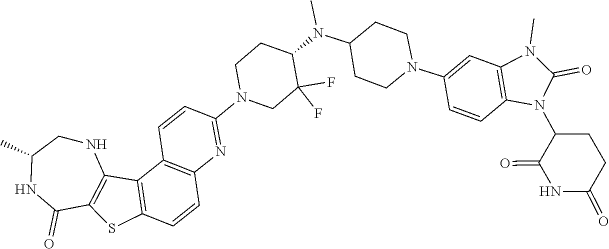 [SMILES: C[C@@H]1CNc2c(sc3ccc4nc(N5CC[C@H](N(C)C6CCN(c7ccc8c(c7)n(C)c(=O)n8C7CCC(=O)NC7=O)CC6)C(F)(F)C5)ccc4c23)C(=O)N1] |
| I-327 | 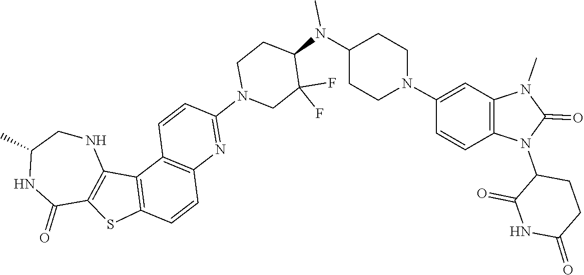 [SMILES: C[C@@H]1CNc2c(sc3ccc4nc(N5CC[C@@H](N(C)C6CCN(c7ccc8c(c7)n(C)c(=O)n8C7CCC(=O)NC7=O)CC6)C(F)(F)C5)ccc4c23)C(=O)N1] |
| I-328 | 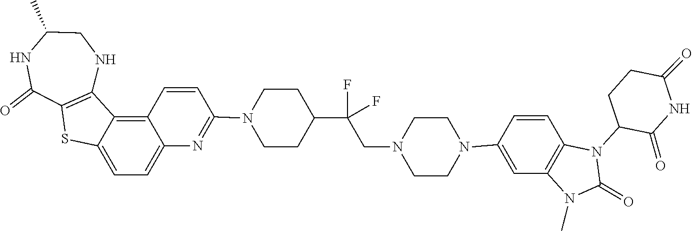 [SMILES: C[C@@H]1CNc2c(sc3ccc4nc(N5CCC(C(F)(F)CN6CCN(c7ccc8c(c7)n(C)c(=O)n8C7CCC(=O)NC7=O)CC6)CC5)ccc4c23)C(=O)N1] |
| I-329 | 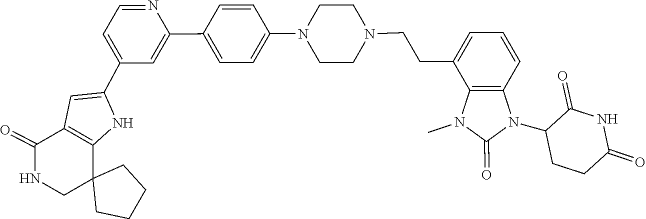 [SMILES: Cn1c(=O)n(C2CCC(=O)NC2=O)c2cccc(CCN3CCN(c4ccc(-c5cc(-c6cc7c([nH]6)C6(CCCC6)CNC7=O)ccn5)cc4)CC3)c21] |
| I-330 | 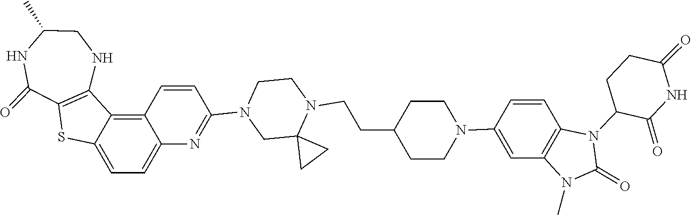 [SMILES: C[C@@H]1CNc2c(sc3ccc4nc(N5CCN(CCC6CCN(c7ccc8c(c7)n(C)c(=O)n8C7CCC(=O)NC7=O)CC6)C6(CC6)C5)ccc4c23)C(=O)N1] |
| I-331 | 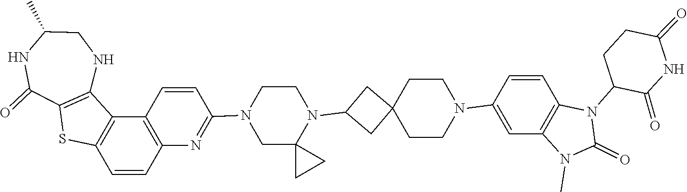 [SMILES: C[C@@H]1CNc2c(sc3ccc4nc(N5CCN(C6CC7(CCN(c8ccc9c(c8)n(C)c(=O)n9C8CCC(=O)NC8=O)CC7)C6)C6(CC6)C5)ccc4c23)C(=O)N1] |
| I-332 | 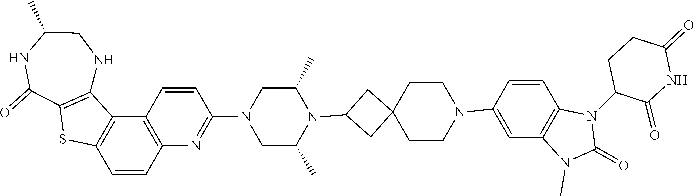 [SMILES: C[C@@H]1CNc2c(sc3ccc4nc(N5C[C@@H](C)N(C6CC7(CCN(c8ccc9c(c8)n(C)c(=O)n9C8CCC(=O)NC8=O)CC7)C6)[C@@H](C)C5)ccc4c23)C(=O)N1] |
| I-333 | 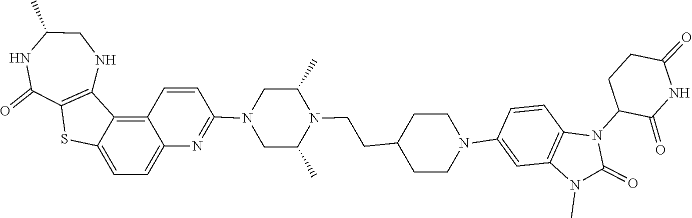 [SMILES: C[C@@H]1CNc2c(sc3ccc4nc(N5C[C@@H](C)N(CCC6CCN(c7ccc8c(c7)n(C)c(=O)n8C7CCC(=O)NC7=O)CC6)[C@@H](C)C5)ccc4c23)C(=O)N1] |
| I-334 | 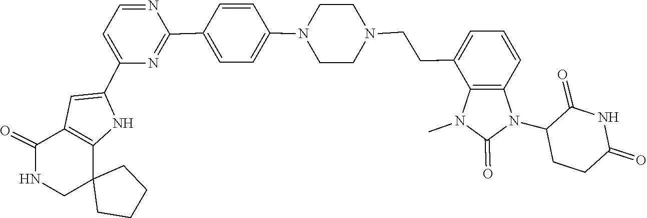 [SMILES: Cn1c(=O)n(C2CCC(=O)NC2=O)c2cccc(CCN3CCN(c4ccc(-c5nccc(-c6cc7c([nH]6)C6(CCCC6)CNC7=O)n5)cc4)CC3)c21] |
| I-335 | 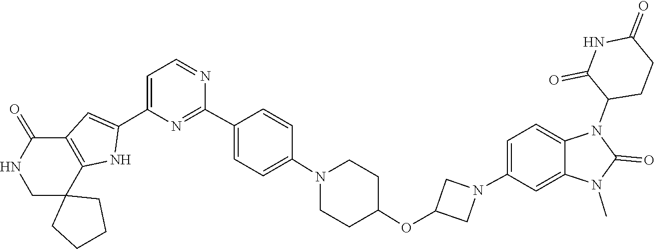 [SMILES: Cn1c(=O)n(C2CCC(=O)NC2=O)c2ccc(N3CC(OC4CCN(c5ccc(-c6nccc(-c7cc8c([nH]7)C7(CCCC7)CNC8=O)n6)cc5)CC4)C3)cc21] |
| I-336 | 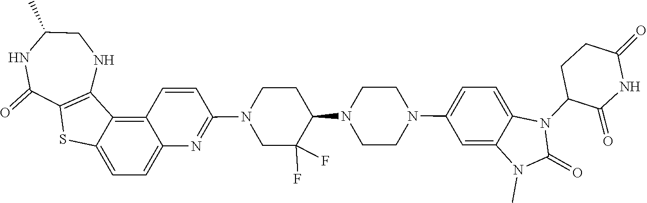 [SMILES: C[C@@H]1CNc2c(sc3ccc4nc(N5CC[C@@H](N6CCN(c7ccc8c(c7)n(C)c(=O)n8C7CCC(=O)NC7=O)CC6)C(F)(F)C5)ccc4c23)C(=O)N1] |
| I-337 | 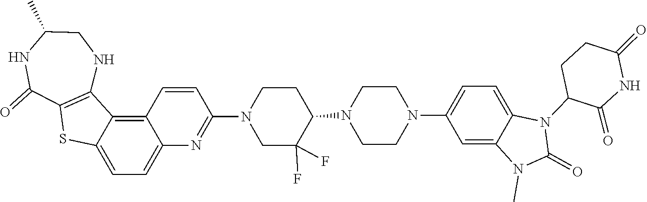 [SMILES: C[C@@H]1CNc2c(sc3ccc4nc(N5CC[C@H](N6CCN(c7ccc8c(c7)n(C)c(=O)n8C7CCC(=O)NC7=O)CC6)C(F)(F)C5)ccc4c23)C(=O)N1] |
| I-338 | 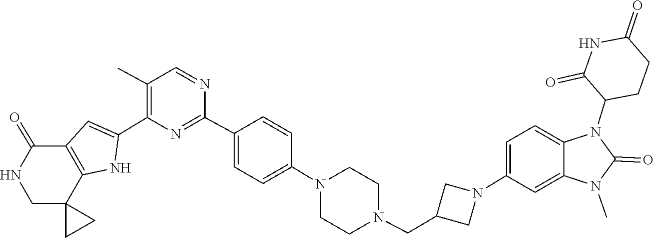 [SMILES: Cc1cnc(-c2ccc(N3CCN(CC4CN(c5ccc6c(c5)n(C)c(=O)n6C5CCC(=O)NC5=O)C4)CC3)cc2)nc1-c1cc2c([nH]1)C1(CC1)CNC2=O] |
| I-339 | 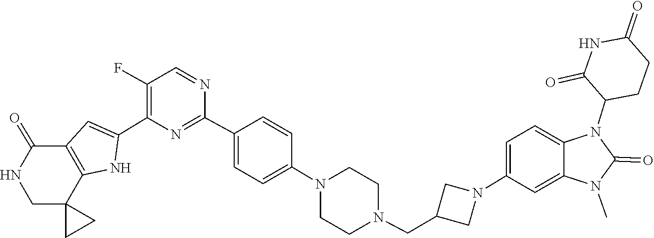 [SMILES: Cn1c(=O)n(C2CCC(=O)NC2=O)c2ccc(N3CC(CN4CCN(c5ccc(-c6ncc(F)c(-c7cc8c([nH]7)C7(CC7)CNC8=O)n6)cc5)CC4)C3)cc21] |
| I-340 | 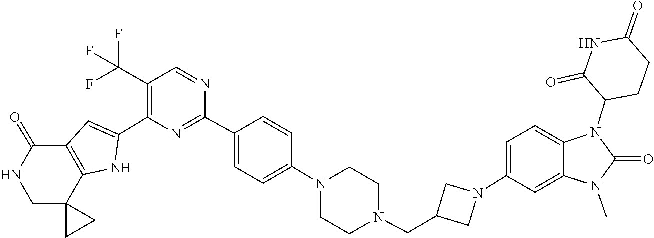 [SMILES: Cn1c(=O)n(C2CCC(=O)NC2=O)c2ccc(N3CC(CN4CCN(c5ccc(-c6ncc(C(F)(F)F)c(-c7cc8c([nH]7)C7(CC7)CNC8=O)n6)cc5)CC4)C3)cc21] |
| I-341 | 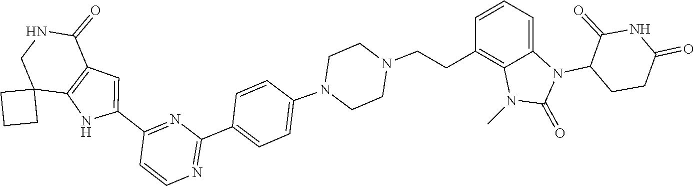 [SMILES: Cn1c(=O)n(C2CCC(=O)NC2=O)c2cccc(CCN3CCN(c4ccc(-c5nccc(-c6cc7c([nH]6)C6(CCC6)CNC7=O)n5)cc4)CC3)c21] |
| I-342 | 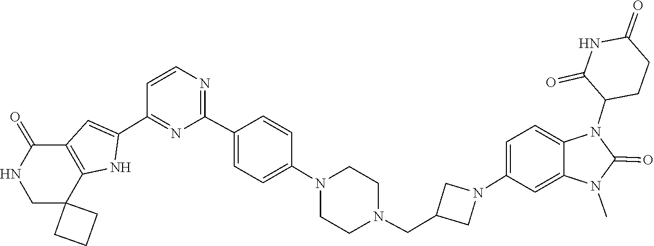 [SMILES: Cn1c(=O)n(C2CCC(=O)NC2=O)c2ccc(N3CC(CN4CCN(c5ccc(-c6nccc(-c7cc8c([nH]7)C7(CCC7)CNC8=O)n6)cc5)CC4)C3)cc21] |
| I-343 | 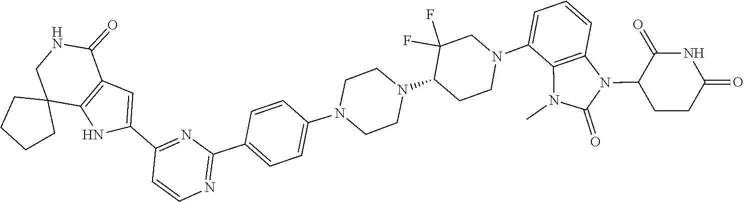 [SMILES: Cn1c(=O)n(C2CCC(=O)NC2=O)c2cccc(N3CC[C@H](N4CCN(c5ccc(-c6nccc(-c7cc8c([nH]7)C7(CCCC7)CNC8=O)n6)cc5)CC4)C(F)(F)C3)c21] |
| I-344 | 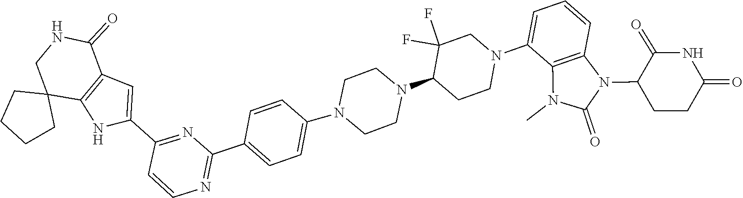 [SMILES: Cn1c(=O)n(C2CCC(=O)NC2=O)c2cccc(N3CC[C@@H](N4CCN(c5ccc(-c6nccc(-c7cc8c([nH]7)C7(CCCC7)CNC8=O)n6)cc5)CC4)C(F)(F)C3)c21] |
| I-345 | 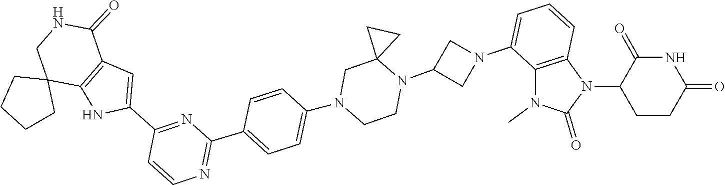 [SMILES: Cn1c(=O)n(C2CCC(=O)NC2=O)c2cccc(N3CC(N4CCN(c5ccc(-c6nccc(-c7cc8c([nH]7)C7(CCCC7)CNC8=O)n6)cc5)CC45CC5)C3)c21] |
| I-346 | 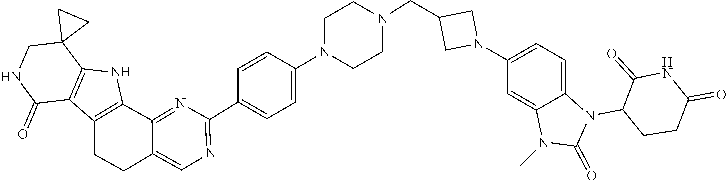 [SMILES: Cn1c(=O)n(C2CCC(=O)NC2=O)c2ccc(N3CC(CN4CCN(c5ccc(-c6ncc7c(n6)-c6[nH]c8c(c6CC7)C(=O)NCC86CC6)cc5)CC4)C3)cc21] |
| I-347 | 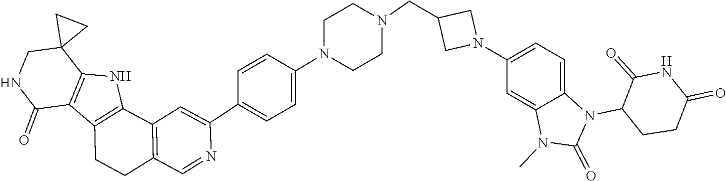 [SMILES: Cn1c(=O)n(C2CCC(=O)NC2=O)c2ccc(N3CC(CN4CCN(c5ccc(-c6cc7c(cn6)CCc6c-7[nH]c7c6C(=O)NCC76CC6)cc5)CC4)C3)cc21] |
| I-348 | 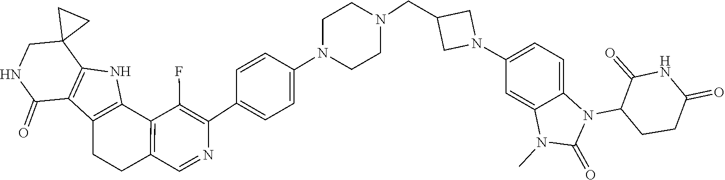 [SMILES: Cn1c(=O)n(C2CCC(=O)NC2=O)c2ccc(N3CC(CN4CCN(c5ccc(-c6ncc7c(c6F)-c6[nH]c8c(c6CC7)C(=O)NCC86CC6)cc5)CC4)C3)cc21] |
| I-349 | 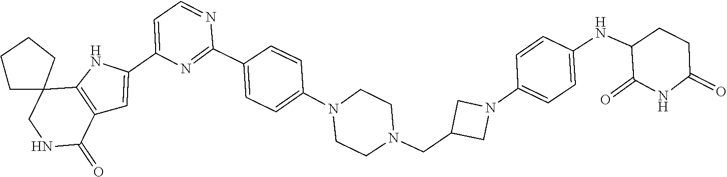 [SMILES: O=C1CCC(Nc2ccc(N3CC(CN4CCN(c5ccc(-c6nccc(-c7cc8c([nH]7)C7(CCCC7)CNC8=O)n6)cc5)CC4)C3)cc2)C(=O)N1] |
| I-350 | 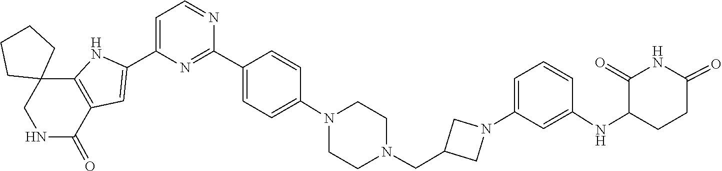 [SMILES: O=C1CCC(Nc2cccc(N3CC(CN4CCN(c5ccc(-c6nccc(-c7cc8c([nH]7)C7(CCCC7)CNC8=O)n6)cc5)CC4)C3)c2)C(=O)N1] |
| I-351 | 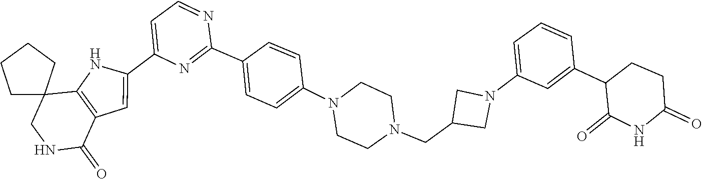 [SMILES: O=C1CCC(c2cccc(N3CC(CN4CCN(c5ccc(-c6nccc(-c7cc8c([nH]7)C7(CCCC7)CNC8=O)n6)cc5)CC4)C3)c2)C(=O)N1] |
| I-352 | 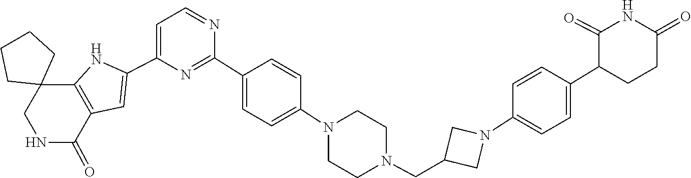 [SMILES: O=C1CCC(c2ccc(N3CC(CN4CCN(c5ccc(-c6nccc(-c7cc8c([nH]7)C7(CCCC7)CNC8=O)n6)cc5)CC4)C3)cc2)C(=O)N1] |
| I-353 | 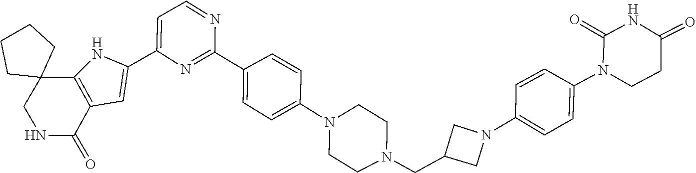 [SMILES: O=C1CCN(c2ccc(N3CC(CN4CCN(c5ccc(-c6nccc(-c7cc8c([nH]7)C7(CCCC7)CNC8=O)n6)cc5)CC4)C3)cc2)C(=O)N1] |
| I-354 | 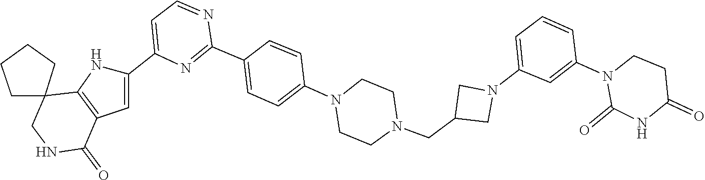 [SMILES: O=C1CCN(c2cccc(N3CC(CN4CCN(c5ccc(-c6nccc(-c7cc8c([nH]7)C7(CCCC7)CNC8=O)n6)cc5)CC4)C3)c2)C(=O)N1] |
| I-355 | 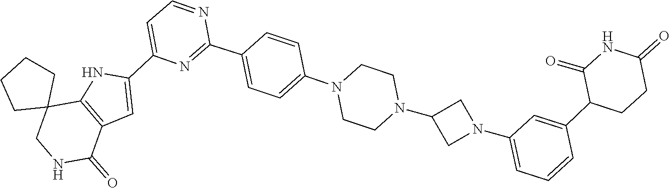 [SMILES: O=C1CCC(c2cccc(N3CC(N4CCN(c5ccc(-c6nccc(-c7cc8c([nH]7)C7(CCCC7)CNC8=O)n6)cc5)CC4)C3)c2)C(=O)N1] |
| I-356 | 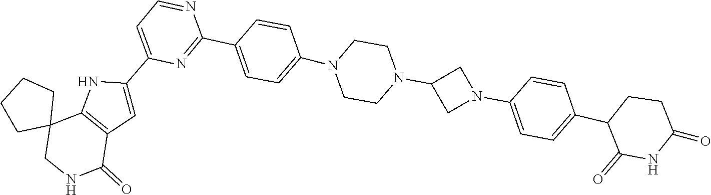 [SMILES: O=C1CCC(c2ccc(N3CC(N4CCN(c5ccc(-c6nccc(-c7cc8c([nH]7)C7(CCCC7)CNC8=O)n6)cc5)CC4)C3)cc2)C(=O)N1] |
| I-357 | 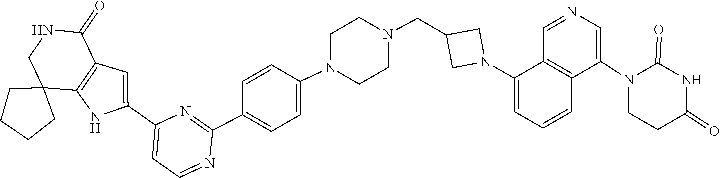 [SMILES: O=C1CCN(c2cncc3c(N4CC(CN5CCN(c6ccc(-c7nccc(-c8cc9c([nH]8)C8(CCCC8)CNC9=O)n7)cc6)CC5)C4)cccc23)C(=O)N1] |
| I-358 | 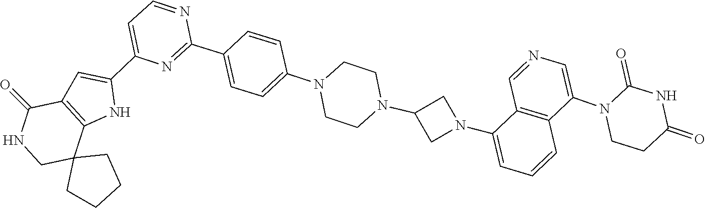 [SMILES: O=C1CCN(c2cncc3c(N4CC(N5CCN(c6ccc(-c7nccc(-c8cc9c([nH]8)C8(CCCC8)CNC9=O)n7)cc6)CC5)C4)cccc23)C(=O)N1] |
| I-359 | 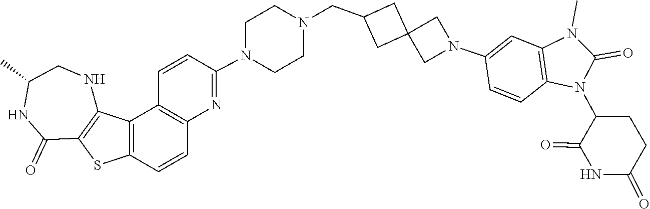 [SMILES: C[C@@H]1CNc2c(sc3ccc4nc(N5CCN(CC6CC7(C6)CN(c6ccc8c(c6)n(C)c(=O)n8C6CCC(=O)NC6=O)C7)CC5)ccc4c23)C(=O)N1] |
| I-360 | 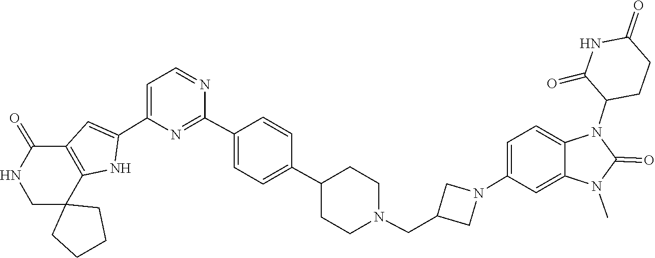 [SMILES: Cn1c(=O)n(C2CCC(=O)NC2=O)c2ccc(N3CC(CN4CCC(c5ccc(-c6nccc(-c7cc8c([nH]7)C7(CCCC7)CNC8=O)n6)cc5)CC4)C3)cc21] |
| I-361 | 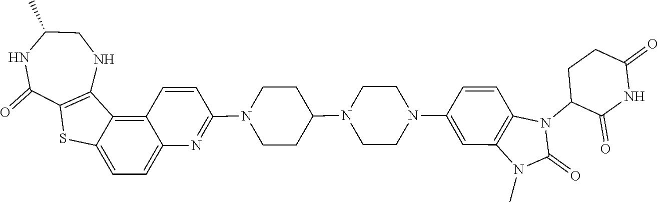 [SMILES: C[C@@H]1CNc2c(sc3ccc4nc(N5CCC(N6CCN(c7ccc8c(c7)n(C)c(=O)n8C7CCC(=O)NC7=O)CC6)CC5)ccc4c23)C(=O)N1] |
| I-362 | 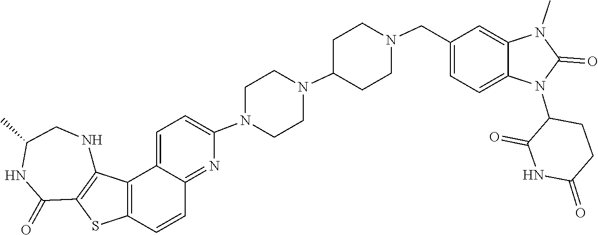 [SMILES: C[C@@H]1CNc2c(sc3ccc4nc(N5CCN(C6CCN(Cc7ccc8c(c7)n(C)c(=O)n8C7CCC(=O)NC7=O)CC6)CC5)ccc4c23)C(=O)N1] |
| I-363 | 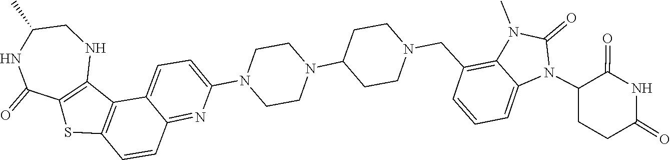 [SMILES: C[C@@H]1CNc2c(sc3ccc4nc(N5CCN(C6CCN(Cc7cccc8c7n(C)c(=O)n8C7CCC(=O)NC7=O)CC6)CC5)ccc4c23)C(=O)N1] |
| I-364 | 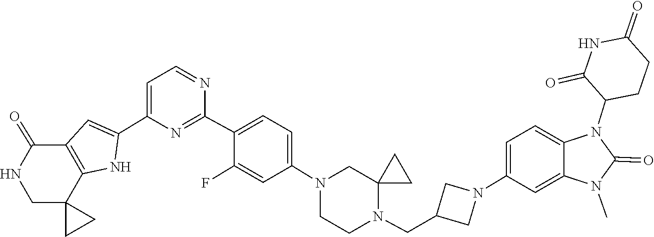 [SMILES: Cn1c(=O)n(C2CCC(=O)NC2=O)c2ccc(N3CC(CN4CCN(c5ccc(-c6nccc(-c7cc8c([nH]7)C7(CC7)CNC8=O)n6)c(F)c5)CC45CC5)C3)cc21] |
| I-365 | 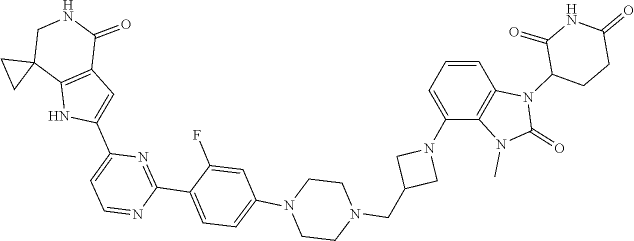 [SMILES: Cn1c(=O)n(C2CCC(=O)NC2=O)c2cccc(N3CC(CN4CCN(c5ccc(-c6nccc(-c7cc8c([nH]7)C7(CC7)CNC8=O)n6)c(F)c5)CC4)C3)c21] |
| I-366 | 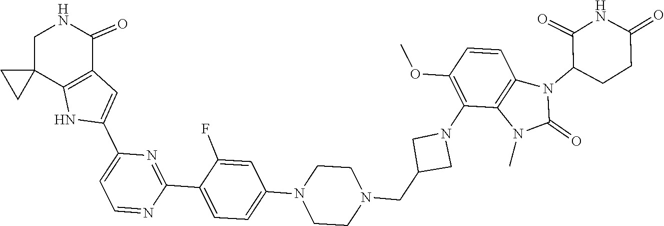 [SMILES: COc1ccc2c(c1N1CC(CN3CCN(c4ccc(-c5nccc(-c6cc7c([nH]6)C6(CC6)CNC7=O)n5)c(F)c4)CC3)C1)n(C)c(=O)n2C1CCC(=O)NC1=O] |
| I-367 | 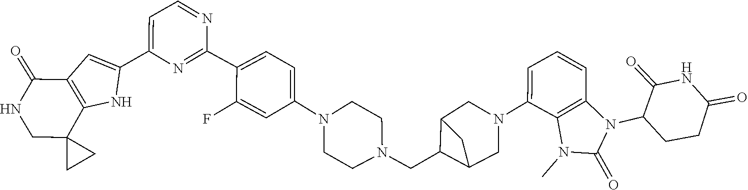 [SMILES: Cn1c(=O)n(C2CCC(=O)NC2=O)c2cccc(N3CC4CC(C3)C4CN3CCN(c4ccc(-c5nccc(-c6cc7c([nH]6)C6(CC6)CNC7=O)n5)c(F)c4)CC3)c21] |
| I-368 | 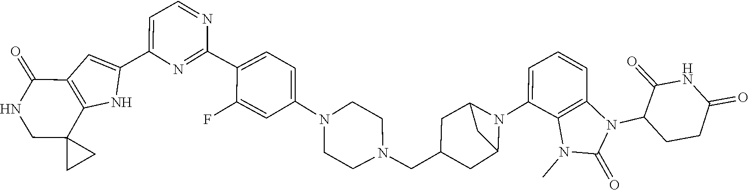 [SMILES: Cn1c(=O)n(C2CCC(=O)NC2=O)c2cccc(N3C4CC(CN5CCN(c6ccc(-c7nccc(-c8cc9c([nH]8)C8(CC8)CNC9=O)n7)c(F)c6)CC5)CC3C4)c21] |
| I-369 | 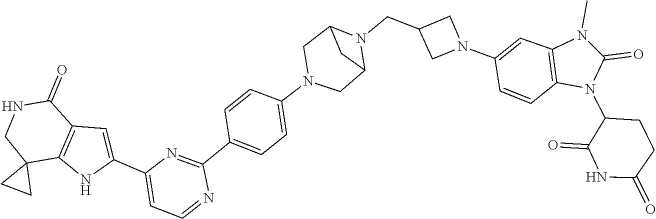 [SMILES: Cn1c(=O)n(C2CCC(=O)NC2=O)c2ccc(N3CC(CN4C5CC4CN(c4ccc(-c6nccc(-c7cc8c([nH]7)C7(CC7)CNC8=O)n6)cc4)C5)C3)cc21] |
| I-370 | 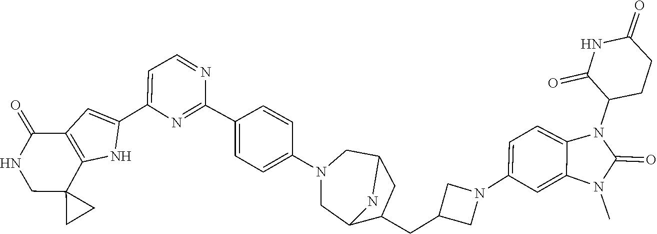 [SMILES: Cn1c(=O)n(C2CCC(=O)NC2=O)c2ccc(N3CC(CC4CC5CN(c6ccc(-c7nccc(-c8cc9c([nH]8)C8(CC8)CNC9=O)n7)cc6)CC4N5)C3)cc21] |
| I-371 | 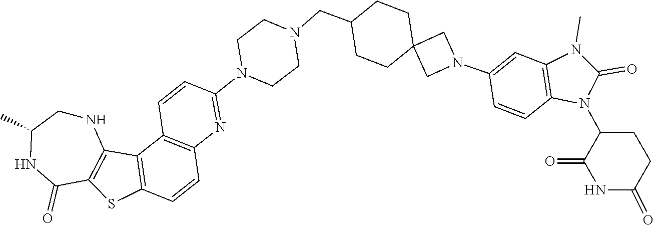 [SMILES: C[C@@H]1CNc2c(sc3ccc4nc(N5CCN(CC6CCC7(CC6)CN(c6ccc8c(c6)n(C)c(=O)n8C6CCC(=O)NC6=O)C7)CC5)ccc4c23)C(=O)N1] |
| I-372 | 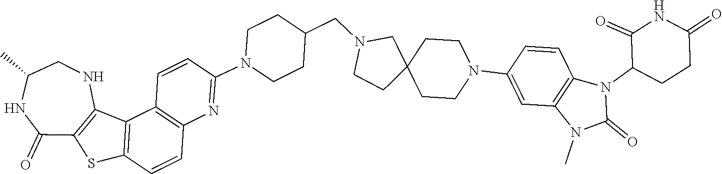 [SMILES: C[C@@H]1CNc2c(sc3ccc4nc(N5CCC(CN6CCC7(CCN(c8ccc9c(c8)n(C)c(=O)n9C8CCC(=O)NC8=O)CC7)C6)CC5)ccc4c23)C(=O)N1] |
| I-373 | 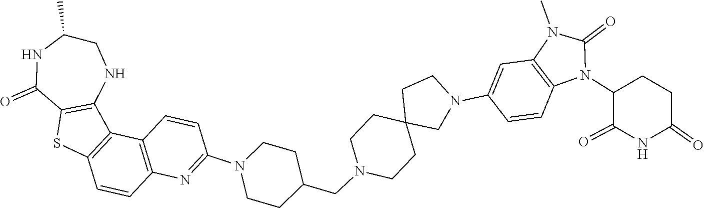 [SMILES: C[C@@H]1CNc2c(sc3ccc4nc(N5CCC(CN6CCC7(CC6)CCN(c6ccc8c(c6)n(C)c(=O)n8C6CCC(=O)NC6=O)C7)CC5)ccc4c23)C(=O)N1] |
| I-374 | 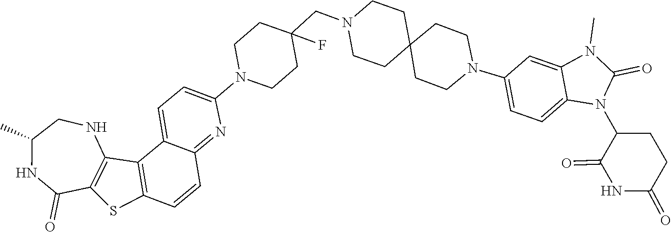 [SMILES: C[C@@H]1CNc2c(sc3ccc4nc(N5CCC(F)(CN6CCC7(CC6)CCN(c6ccc8c(c6)n(C)c(=O)n8C6CCC(=O)NC6=O)CC7)CC5)ccc4c23)C(=O)N1] |
| I-375 | 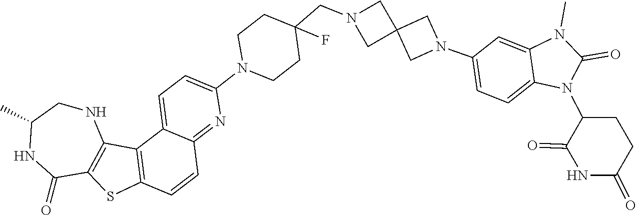 [SMILES: C[C@@H]1CNc2c(sc3ccc4nc(N5CCC(F)(CN6CC7(C6)CN(c6ccc8c(c6)n(C)c(=O)n8C6CCC(=O)NC6=O)C7)CC5)ccc4c23)C(=O)N1] |
| I-376 | 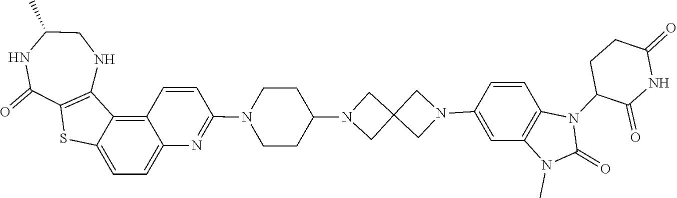 [SMILES: C[C@@H]1CNc2c(sc3ccc4nc(N5CCC(N6CC7(CN(c8ccc9c(c8)n(C)c(=O)n9C8CCC(=O)NC8=O)C7)C6)CC5)ccc4c23)C(=O)N1] |
| I-377 | 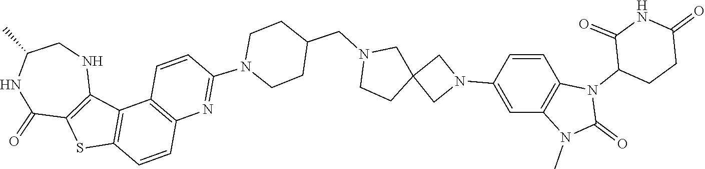 [SMILES: C[C@@H]1CNc2c(sc3ccc4nc(N5CCC(CN6CCC7(C6)CN(c6ccc8c(c6)n(C)c(=O)n8C6CCC(=O)NC6=O)C7)CC5)ccc4c23)C(=O)N1] |
| I-378 | 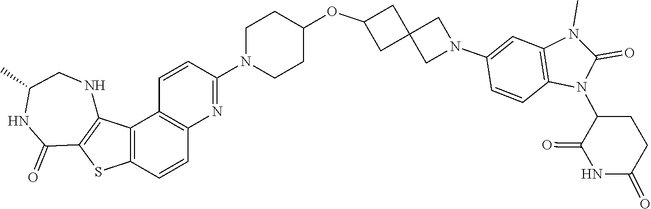 [SMILES: C[C@@H]1CNc2c(sc3ccc4nc(N5CCC(OC6CC7(C6)CN(c6ccc8c(c6)n(C)c(=O)n8C6CCC(=O)NC6=O)C7)CC5)ccc4c23)C(=O)N1] |
| I-379 | 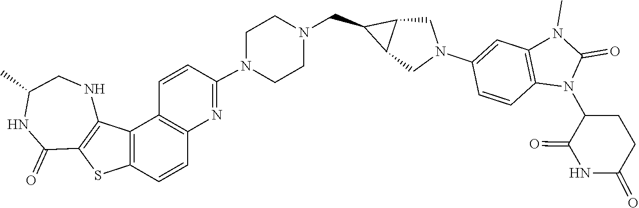 [SMILES: C[C@@H]1CNc2c(sc3ccc4nc(N5CCN(C[C@@H]6[C@H]7CN(c8ccc9c(c8)n(C)c(=O)n9C8CCC(=O)NC8=O)C[C@@H]67)CC5)ccc4c23)C(=O)N1] |
| I-380 | 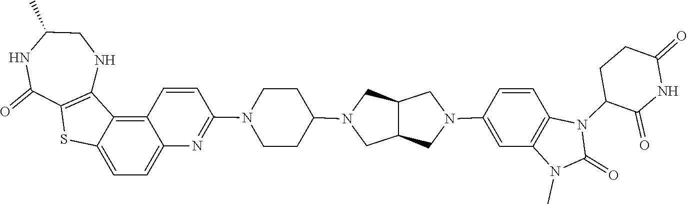 [SMILES: C[C@@H]1CNc2c(sc3ccc4nc(N5CCC(N6C[C@H]7CN(c8ccc9c(c8)n(C)c(=O)n9C8CCC(=O)NC8=O)C[C@H]7C6)CC5)ccc4c23)C(=O)N1] |
| I-381 | 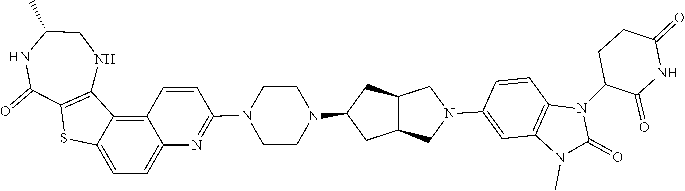 [SMILES: C[C@@H]1CNc2c(sc3ccc4nc(N5CCN([C@@H]6C[C@@H]7CN(c8ccc9c(c8)n(C)c(=O)n9C8CCC(=O)NC8=O)C[C@@H]7C6)CC5)ccc4c23)C(=O)N1] |
| I-382 | 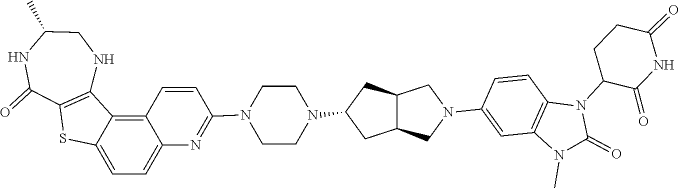 [SMILES: C[C@@H]1CNc2c(sc3ccc4nc(N5CCN([C@H]6C[C@@H]7CN(c8ccc9c(c8)n(C)c(=O)n9C8CCC(=O)NC8=O)C[C@@H]7C6)CC5)ccc4c23)C(=O)N1] |
| I-383 | 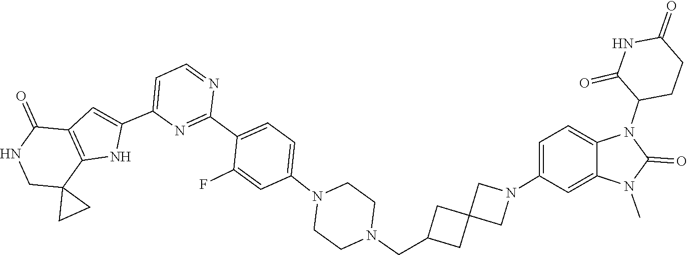 [SMILES: Cn1c(=O)n(C2CCC(=O)NC2=O)c2ccc(N3CC4(CC(CN5CCN(c6ccc(-c7nccc(-c8cc9c([nH]8)C8(CC8)CNC9=O)n7)c(F)c6)CC5)C4)C3)cc21] |
| I-384 | 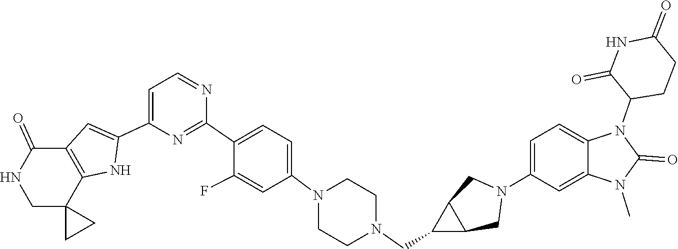 [SMILES: Cn1c(=O)n(C2CCC(=O)NC2=O)c2ccc(N3C[C@@H]4[C@@H](CN5CCN(c6ccc(-c7nccc(-c8cc9c([nH]8)C8(CC8)CNC9=O)n7)c(F)c6)CC5)[C@@H]4C3)cc21] |
| I-385 | 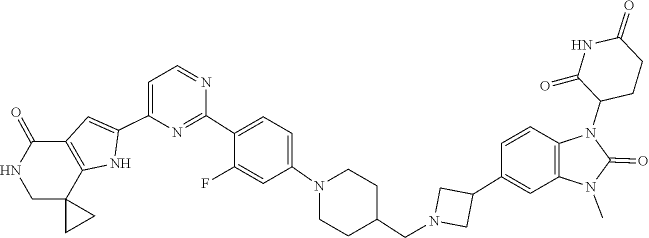 [SMILES: Cn1c(=O)n(C2CCC(=O)NC2=O)c2ccc(C3CN(CC4CCN(c5ccc(-c6nccc(-c7cc8c([nH]7)C7(CC7)CNC8=O)n6)c(F)c5)CC4)C3)cc21] |
| I-386 | 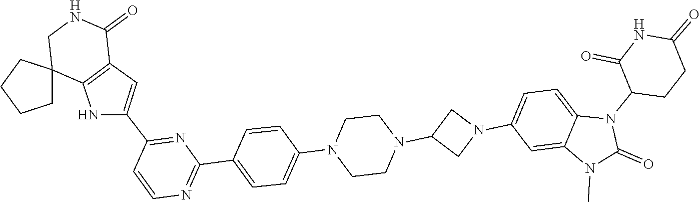 [SMILES: Cn1c(=O)n(C2CCC(=O)NC2=O)c2ccc(N3CC(N4CCN(c5ccc(-c6nccc(-c7cc8c([nH]7)C7(CCCC7)CNC8=O)n6)cc5)CC4)C3)cc21] |
| I-387 | 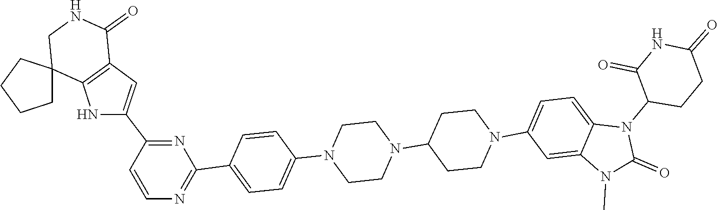 [SMILES: Cn1c(=O)n(C2CCC(=O)NC2=O)c2ccc(N3CCC(N4CCN(c5ccc(-c6nccc(-c7cc8c([nH]7)C7(CCCC7)CNC8=O)n6)cc5)CC4)CC3)cc21] |
| I-388 | 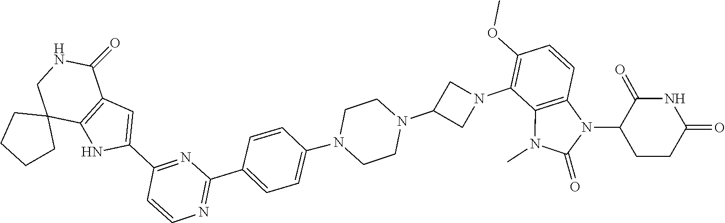 [SMILES: COc1ccc2c(c1N1CC(N3CCN(c4ccc(-c5nccc(-c6cc7c([nH]6)C6(CCCC6)CNC7=O)n5)cc4)CC3)C1)n(C)c(=O)n2C1CCC(=O)NC1=O] |
| I-389 | 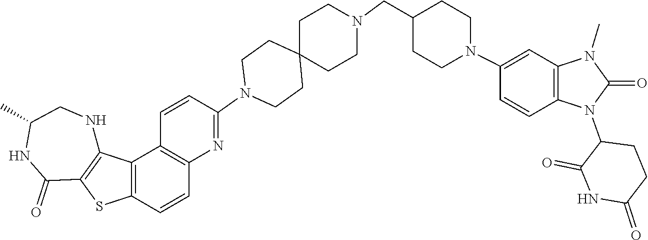 [SMILES: C[C@@H]1CNc2c(sc3ccc4nc(N5CCC6(CCN(CC7CCN(c8ccc9c(c8)n(C)c(=O)n9C8CCC(=O)NC8=O)CC7)CC6)CC5)ccc4c23)C(=O)N1] |
| I-390 | 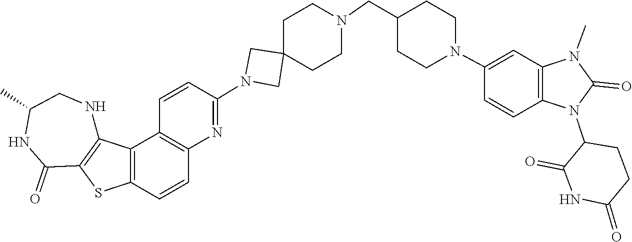 [SMILES: C[C@@H]1CNc2c(sc3ccc4nc(N5CC6(CCN(CC7CCN(c8ccc9c(c8)n(C)c(=O)n9C8CCC(=O)NC8=O)CC7)CC6)C5)ccc4c23)C(=O)N1] |
| I-391 | 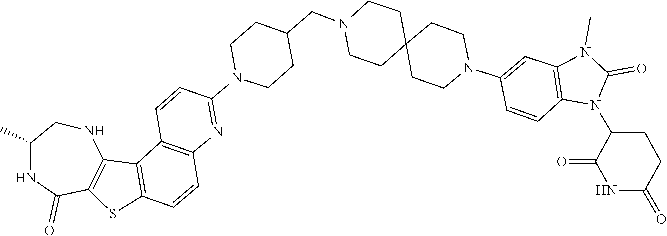 [SMILES: C[C@@H]1CNc2c(sc3ccc4nc(N5CCC(CN6CCC7(CC6)CCN(c6ccc8c(c6)n(C)c(=O)n8C6CCC(=O)NC6=O)CC7)CC5)ccc4c23)C(=O)N1] |
| I-392 | 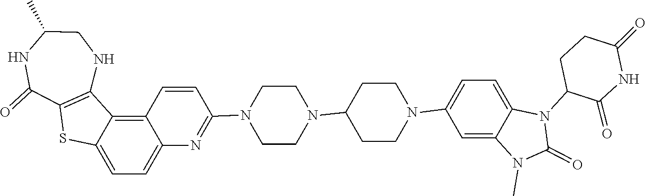 [SMILES: C[C@@H]1CNc2c(sc3ccc4nc(N5CCN(C6CCN(c7ccc8c(c7)n(C)c(=O)n8C7CCC(=O)NC7=O)CC6)CC5)ccc4c23)C(=O)N1] |
| I-393 | 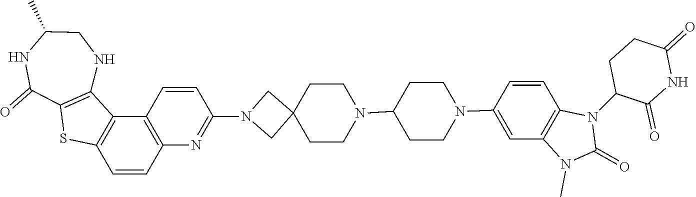 [SMILES: C[C@@H]1CNc2c(sc3ccc4nc(N5CC6(CCN(C7CCN(c8ccc9c(c8)n(C)c(=O)n9C8CCC(=O)NC8=O)CC7)CC6)C5)ccc4c23)C(=O)N1] |
| I-394 | 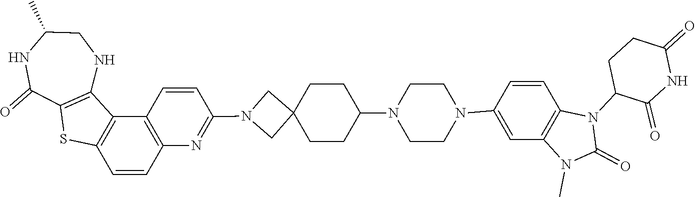 [SMILES: C[C@@H]1CNc2c(sc3ccc4nc(N5CC6(CCC(N7CCN(c8ccc9c(c8)n(C)c(=O)n9C8CCC(=O)NC8=O)CC7)CC6)C5)ccc4c23)C(=O)N1] |
| I-395 | 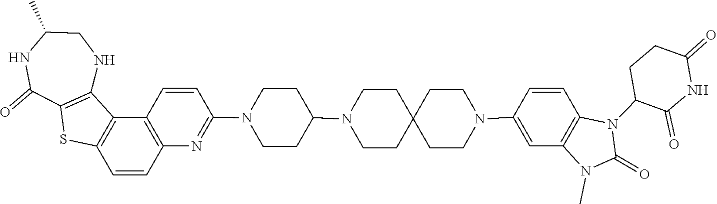 [SMILES: C[C@@H]1CNc2c(sc3ccc4nc(N5CCC(N6CCC7(CCN(c8ccc9c(c8)n(C)c(=O)n9C8CCC(=O)NC8=O)CC7)CC6)CC5)ccc4c23)C(=O)N1] |
| I-396 | 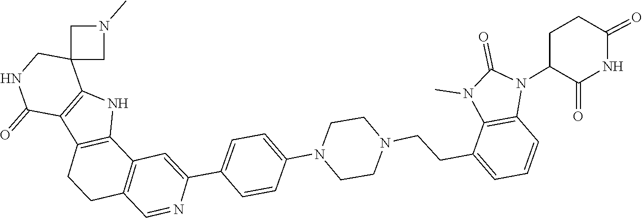 [SMILES: CN1CC2(CNC(=O)c3c2[nH]c2c3CCc3cnc(-c4ccc(N5CCN(CCc6cccc7c6n(C)c(=O)n7C6CCC(=O)NC6=O)CC5)cc4)cc3-2)C1] |
| I-397 | 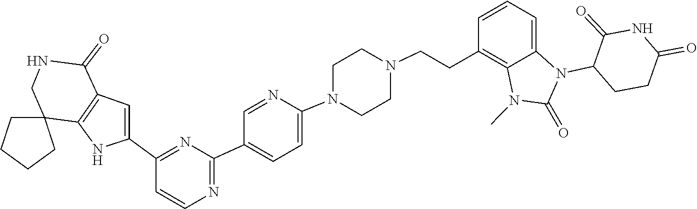 [SMILES: Cn1c(=O)n(C2CCC(=O)NC2=O)c2cccc(CCN3CCN(c4ccc(-c5nccc(-c6cc7c([nH]6)C6(CCCC6)CNC7=O)n5)cn4)CC3)c21] |
| I-398 | 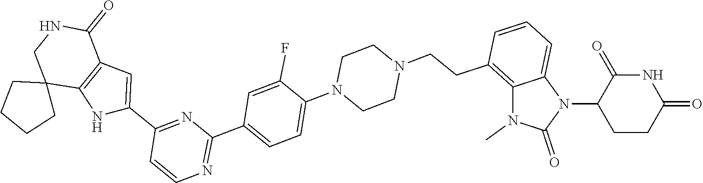 [SMILES: Cn1c(=O)n(C2CCC(=O)NC2=O)c2cccc(CCN3CCN(c4ccc(-c5nccc(-c6cc7c([nH]6)C6(CCCC6)CNC7=O)n5)cc4F)CC3)c21] |
| I-399 | 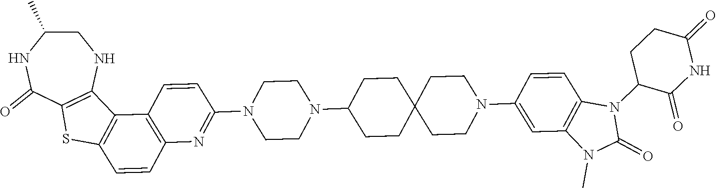 [SMILES: C[C@@H]1CNc2c(sc3ccc4nc(N5CCN(C6CCC7(CC6)CCN(c6ccc8c(c6)n(C)c(=O)n8C6CCC(=O)NC6=O)CC7)CC5)ccc4c23)C(=O)N1] |
| I-400 | 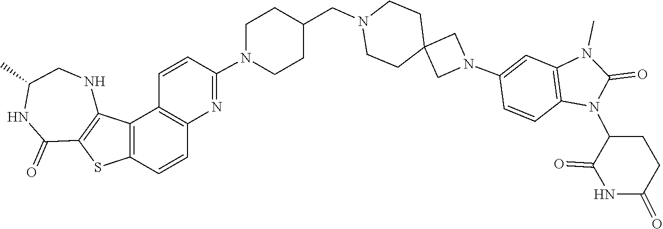 [SMILES: C[C@@H]1CNc2c(sc3ccc4nc(N5CCC(CN6CCC7(CC6)CN(c6ccc8c(c6)n(C)c(=O)n8C6CCC(=O)NC6=O)C7)CC5)ccc4c23)C(=O)N1] |
| I-401 | 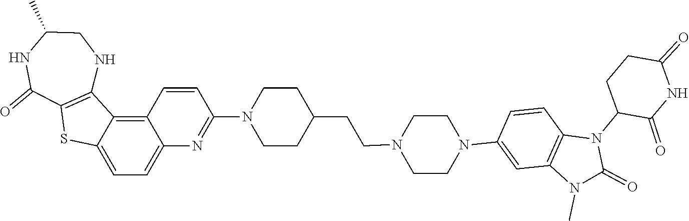 [SMILES: C[C@@H]1CNc2c(sc3ccc4nc(N5CCC(CCN6CCN(c7ccc8c(c7)n(C)c(=O)n8C7CCC(=O)NC7=O)CC6)CC5)ccc4c23)C(=O)N1] |
| I-402 | 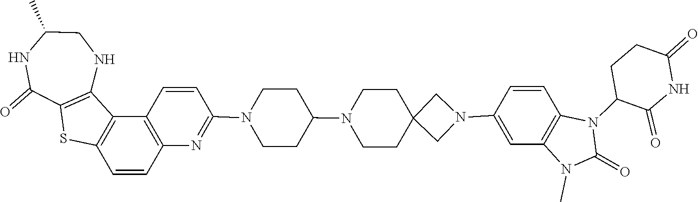 [SMILES: C[C@@H]1CNc2c(sc3ccc4nc(N5CCC(N6CCC7(CC6)CN(c6ccc8c(c6)n(C)c(=O)n8C6CCC(=O)NC6=O)C7)CC5)ccc4c23)C(=O)N1] |
| I-403 | 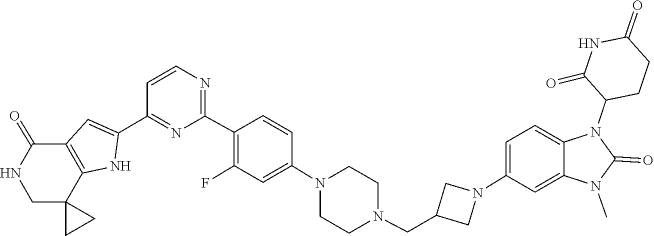 [SMILES: Cn1c(=O)n(C2CCC(=O)NC2=O)c2ccc(N3CC(CN4CCN(c5ccc(-c6nccc(-c7cc8c([nH]7)C7(CC7)CNC8=O)n6)c(F)c5)CC4)C3)cc21] |
| I-404 | 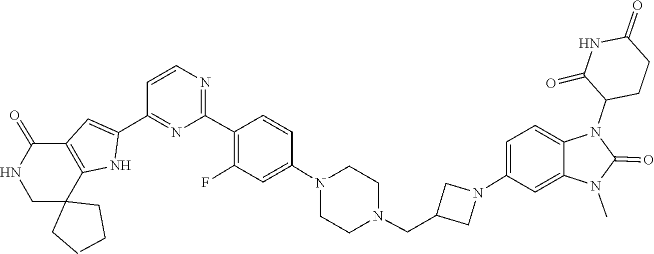 [SMILES: CCCC1(CC)CNC(=O)c2cc(-c3ccnc(-c4ccc(N5CCN(CC6CN(c7ccc8c(c7)n(C)c(=O)n8C7CCC(=O)NC7=O)C6)CC5)cc4F)n3)[nH]c21] |
| I-405 | 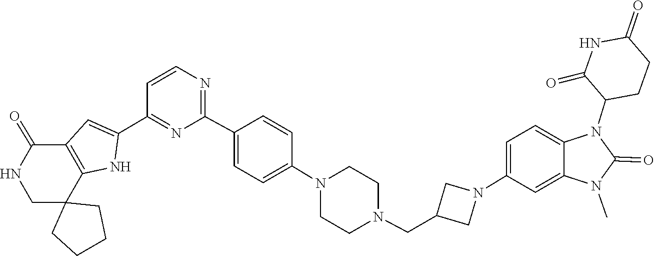 [SMILES: Cn1c(=O)n(C2CCC(=O)NC2=O)c2ccc(N3CC(CN4CCN(c5ccc(-c6nccc(-c7cc8c([nH]7)C7(CCCC7)CNC8=O)n6)cc5)CC4)C3)cc21] |
| I-406 | 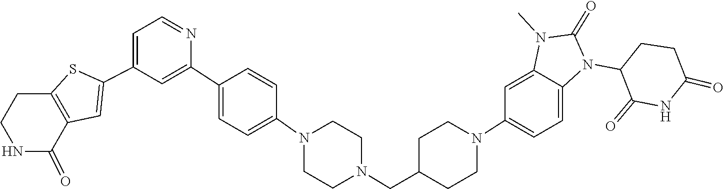 [SMILES: Cn1c(=O)n(C2CCC(=O)NC2=O)c2ccc(N3CCC(CN4CCN(c5ccc(-c6cc(-c7cc8c(s7)CCNC8=O)ccn6)cc5)CC4)CC3)cc21] |
| I-407 | 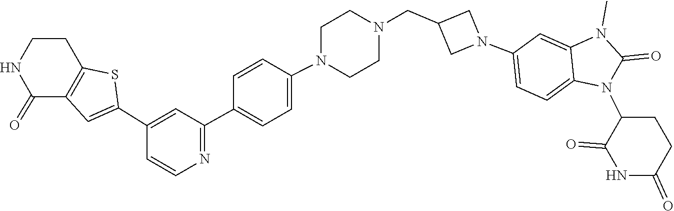 [SMILES: Cn1c(=O)n(C2CCC(=O)NC2=O)c2ccc(N3CC(CN4CCN(c5ccc(-c6cc(-c7cc8c(s7)CCNC8=O)ccn6)cc5)CC4)C3)cc21] |
| I-408 | 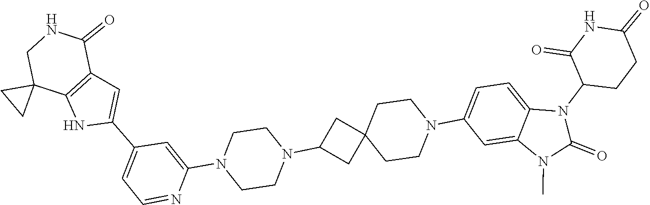 [SMILES: Cn1c(=O)n(C2CCC(=O)NC2=O)c2ccc(N3CCC4(CC3)CC(N3CCN(c5cc(-c6cc7c([nH]6)C6(CC6)CNC7=O)ccn5)CC3)C4)cc21] |
| I-409 | 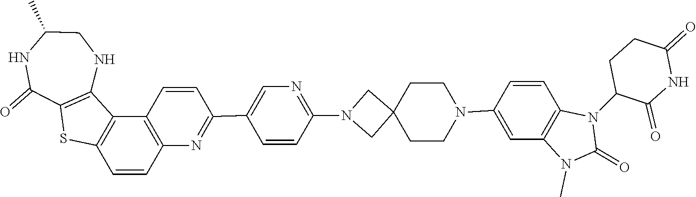 [SMILES: C[C@@H]1CNc2c(sc3ccc4nc(-c5ccc(N6CC7(CCN(c8ccc9c(c8)n(C)c(=O)n9C8CCC(=O)NC8=O)CC7)C6)nc5)ccc4c23)C(=O)N1] |
| I-410 | 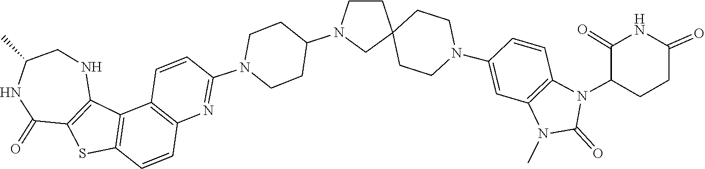 [SMILES: C[C@@H]1CNc2c(sc3ccc4nc(N5CCC(N6CCC7(CCN(c8ccc9c(c8)n(C)c(=O)n9C8CCC(=O)NC8=O)CC7)C6)CC5)ccc4c23)C(=O)N1] |
| I-411 | 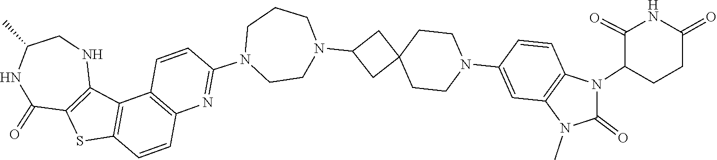 [SMILES: C[C@@H]1CNc2c(sc3ccc4nc(N5CCCN(C6CC7(CCN(c8ccc9c(c8)n(C)c(=O)n9C8CCC(=O)NC8=O)CC7)C6)CC5)ccc4c23)C(=O)N1] |
| I-412 | 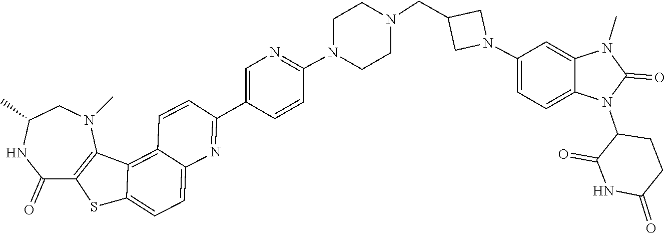 [SMILES: C[C@@H]1CN(C)c2c(sc3ccc4nc(-c5ccc(N6CCN(CC7CN(c8ccc9c(c8)n(C)c(=O)n9C8CCC(=O)NC8=O)C7)CC6)nc5)ccc4c23)C(=O)N1] |
| I-413 | 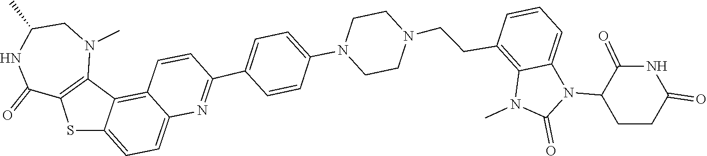 [SMILES: C[C@@H]1CN(C)c2c(sc3ccc4nc(-c5ccc(N6CCN(CCc7cccc8c7n(C)c(=O)n8C7CCC(=O)NC7=O)CC6)cc5)ccc4c23)C(=O)N1] |
| I-414 | 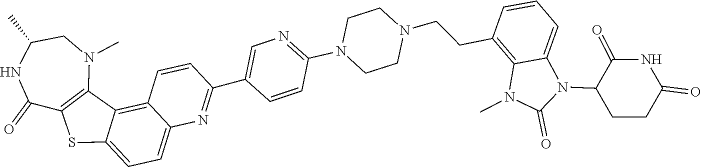 [SMILES: C[C@@H]1CN(C)c2c(sc3ccc4nc(-c5ccc(N6CCN(CCc7cccc8c7n(C)c(=O)n8C7CCC(=O)NC7=O)CC6)nc5)ccc4c23)C(=O)N1] |
| I-415 | 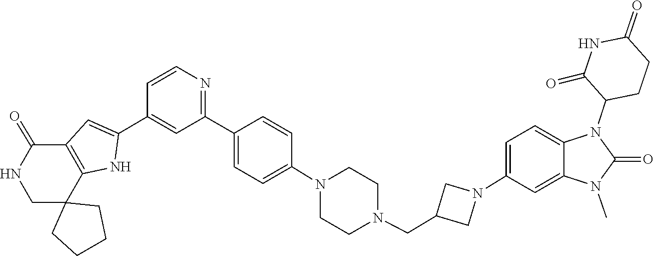 [SMILES: Cn1c(=O)n(C2CCC(=O)NC2=O)c2ccc(N3CC(CN4CCN(c5ccc(-c6cc(-c7cc8c([nH]7)C7(CCCC7)CNC8=O)ccn6)cc5)CC4)C3)cc21] |
| I-416 | 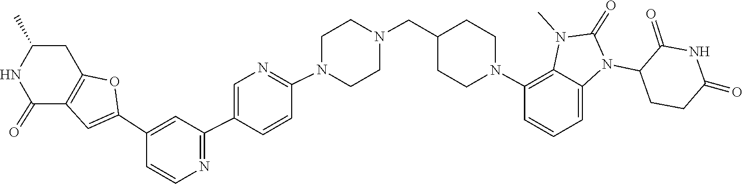 [SMILES: C[C@@H]1Cc2oc(-c3ccnc(-c4ccc(N5CCN(CC6CCN(c7cccc8c7n(C)c(=O)n8C7CCC(=O)NC7=O)CC6)CC5)nc4)c3)cc2C(=O)N1] |
| I-417 | 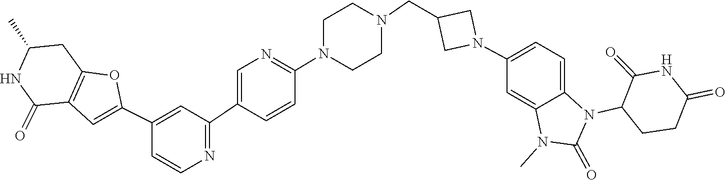 [SMILES: C[C@@H]1Cc2oc(-c3ccnc(-c4ccc(N5CCN(CC6CN(c7ccc8c(c7)n(C)c(=O)n8C7CCC(=O)NC7=O)C6)CC5)nc4)c3)cc2C(=O)N1] |
| I-418 | 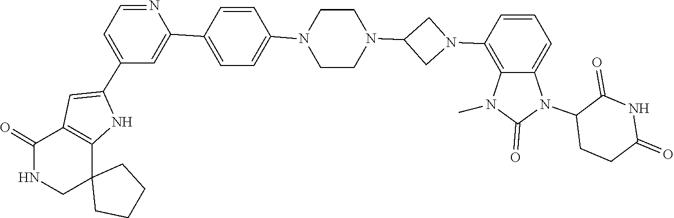 [SMILES: Cn1c(=O)n(C2CCC(=O)NC2=O)c2cccc(N3CC(N4CCN(c5ccc(-c6cc(-c7cc8c([nH]7)C7(CCCC7)CNC8=O)ccn6)cc5)CC4)C3)c21] |
| I-419 | 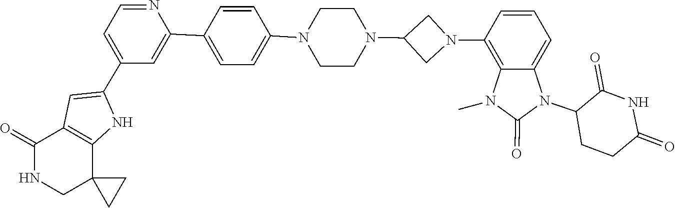 [SMILES: Cn1c(=O)n(C2CCC(=O)NC2=O)c2cccc(N3CC(N4CCN(c5ccc(-c6cc(-c7cc8c([nH]7)C7(CC7)CNC8=O)ccn6)cc5)CC4)C3)c21] |
| I-420 | 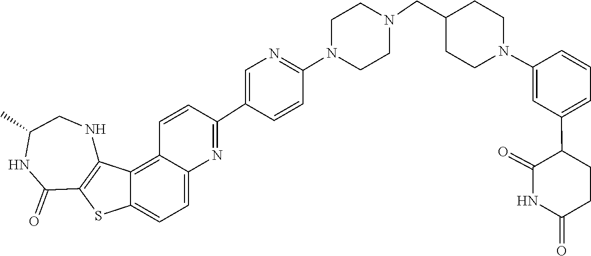 [SMILES: C[C@@H]1CNc2c(sc3ccc4nc(-c5ccc(N6CCN(CC7CCN(c8cccc(C9CCC(=O)NC9=O)c8)CC7)CC6)nc5)ccc4c23)C(=O)N1] |
| I-421 | 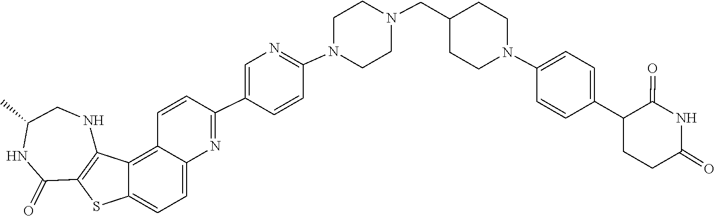 [SMILES: C[C@@H]1CNc2c(sc3ccc4nc(-c5ccc(N6CCN(CC7CCN(c8ccc(C9CCC(=O)NC9=O)cc8)CC7)CC6)nc5)ccc4c23)C(=O)N1] |
| I-422 | 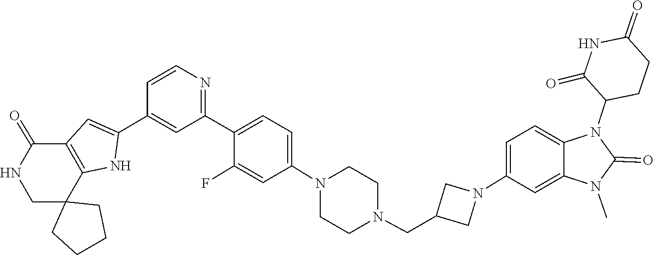 [SMILES: Cn1c(=O)n(C2CCC(=O)NC2=O)c2ccc(N3CC(CN4CCN(c5ccc(-c6cc(-c7cc8c([nH]7)C7(CCCC7)CNC8=O)ccn6)c(F)c5)CC4)C3)cc21] |
| I-423 | 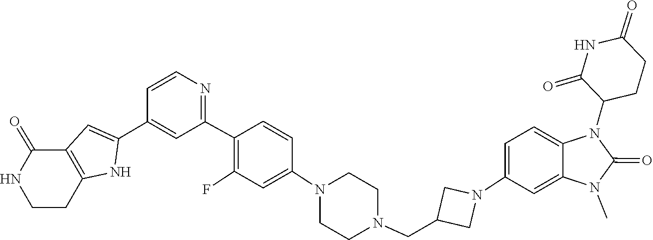 [SMILES: Cn1c(=O)n(C2CCC(=O)NC2=O)c2ccc(N3CC(CN4CCN(c5ccc(-c6cc(-c7cc8c([nH]7)CCNC8=O)ccn6)c(F)c5)CC4)C3)cc21] |
| I-424 | 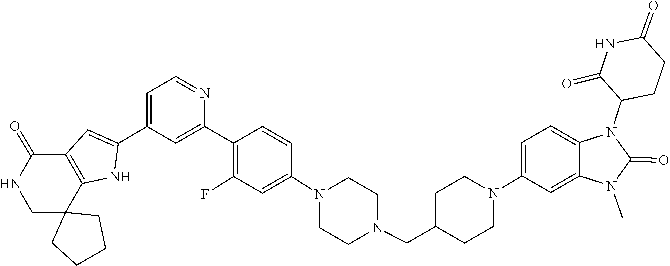 [SMILES: Cn1c(=O)n(C2CCC(=O)NC2=O)c2ccc(N3CCC(CN4CCN(c5ccc(-c6cc(-c7cc8c([nH]7)C7(CCCC7)CNC8=O)ccn6)c(F)c5)CC4)CC3)cc21] |
| I-425 | 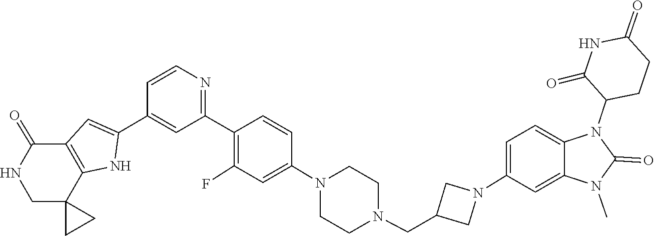 [SMILES: Cn1c(=O)n(C2CCC(=O)NC2=O)c2ccc(N3CC(CN4CCN(c5ccc(-c6cc(-c7cc8c([nH]7)C7(CC7)CNC8=O)ccn6)c(F)c5)CC4)C3)cc21] |
| I-426 | 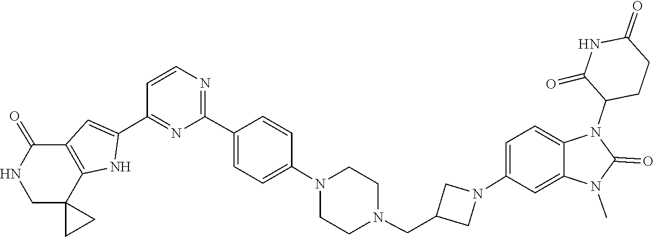 [SMILES: Cn1c(=O)n(C2CCC(=O)NC2=O)c2ccc(N3CC(CN4CCN(c5ccc(-c6nccc(-c7cc8c([nH]7)C7(CC7)CNC8=O)n6)cc5)CC4)C3)cc21] |
| I-427 | 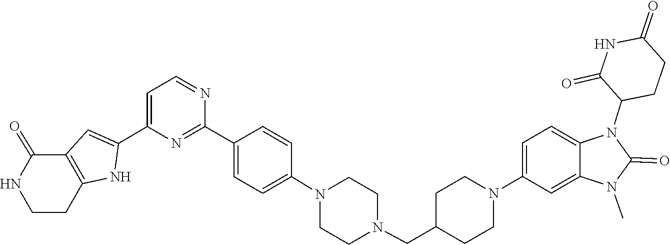 [SMILES: Cn1c(=O)n(C2CCC(=O)NC2=O)c2ccc(N3CCC(CN4CCN(c5ccc(-c6nccc(-c7cc8c([nH]7)CCNC8=O)n6)cc5)CC4)CC3)cc21] |
| I-428 | 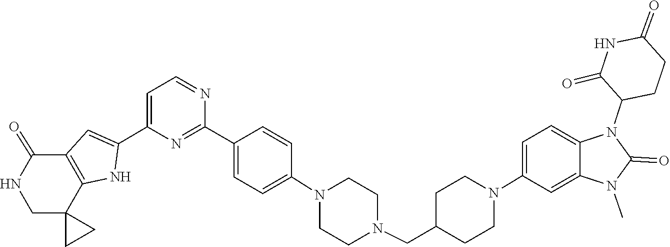 [SMILES: Cn1c(=O)n(C2CCC(=O)NC2=O)c2ccc(N3CCC(CN4CCN(c5ccc(-c6nccc(-c7cc8c([nH]7)C7(CC7)CNC8=O)n6)cc5)CC4)CC3)cc21] |
| I-429 | 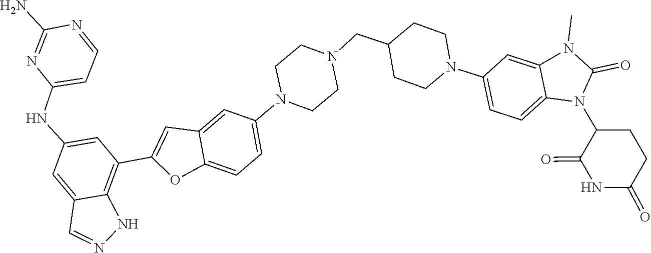 [SMILES: Cn1c(=O)n(C2CCC(=O)NC2=O)c2ccc(N3CCC(CN4CCN(c5ccc6oc(-c7cc(Nc8ccnc(N)n8)cc8cn[nH]c78)cc6c5)CC4)CC3)cc21] |
| I-430 | 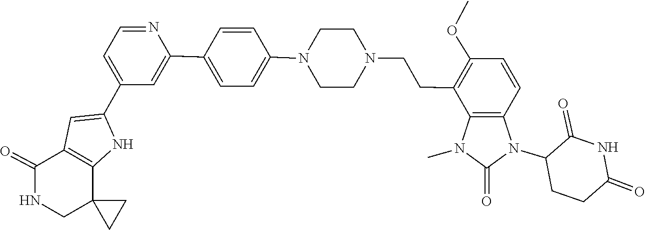 [SMILES: COc1ccc2c(c1CCN1CCN(c3ccc(-c4cc(-c5cc6c([nH]5)C5(CC5)CNC6=O)ccn4)cc3)CC1)n(C)c(=O)n2C1CCC(=O)NC1=O] |
| I-431 | 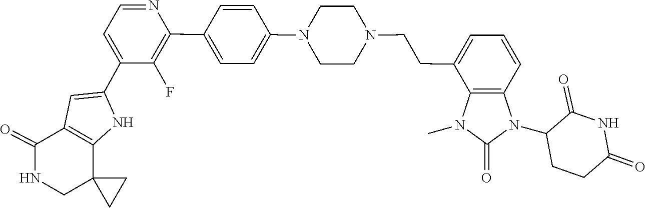 [SMILES: Cn1c(=O)n(C2CCC(=O)NC2=O)c2cccc(CCN3CCN(c4ccc(-c5nccc(-c6cc7c([nH]6)C6(CC6)CNC7=O)c5F)cc4)CC3)c21] |
| I-432 | 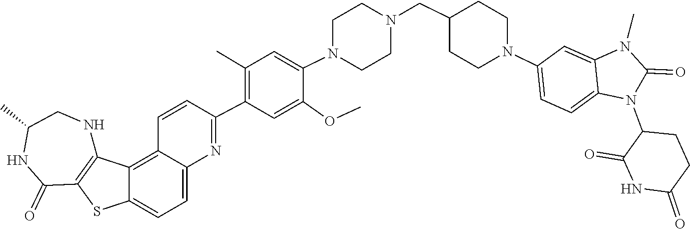 [SMILES: COc1cc(-c2ccc3c(ccc4sc5c(c43)NC[C@@H](C)NC5=O)n2)c(C)cc1N1CCN(CC2CCN(c3ccc4c(c3)n(C)c(=O)n4C3CCC(=O)NC3=O)CC2)CC1] |
| I-433 | 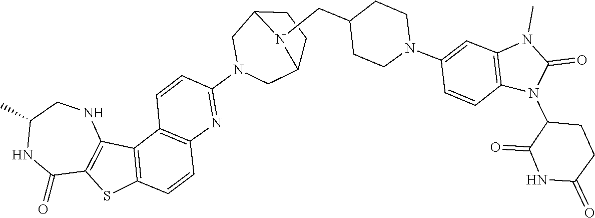 [SMILES: C[C@@H]1CNc2c(sc3ccc4nc(N5CC6CCC(C5)N6CC5CCN(c6ccc7c(c6)n(C)c(=O)n7C6CCC(=O)NC6=O)CC5)ccc4c23)C(=O)N1] |
| I-434 | 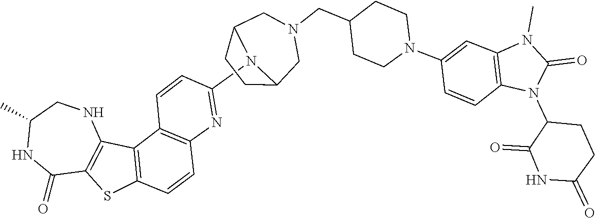 [SMILES: C[C@@H]1CNc2c(sc3ccc4nc(N5C6CCC5CN(CC5CCN(c7ccc8c(c7)n(C)c(=O)n8C7CCC(=O)NC7=O)CC5)C6)ccc4c23)C(=O)N1] |
| I-435 | 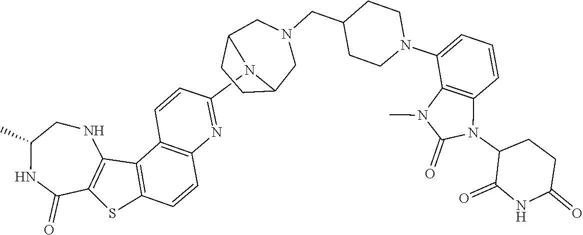 [SMILES: C[C@@H]1CNc2c(sc3ccc4nc(N5C6CCC5CN(CC5CCN(c7cccc8c7n(C)c(=O)n8C7CCC(=O)NC7=O)CC5)C6)ccc4c23)C(=O)N1] |
| I-436 | 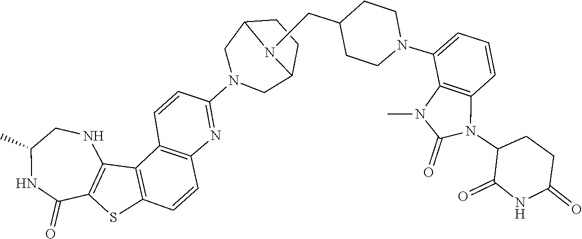 [SMILES: C[C@@H]1CNc2c(sc3ccc4nc(N5CC6CCC(C5)N6CC5CCN(c6cccc7c6n(C)c(=O)n7C6CCC(=O)NC6=O)CC5)ccc4c23)C(=O)N1] |
| I-437 | 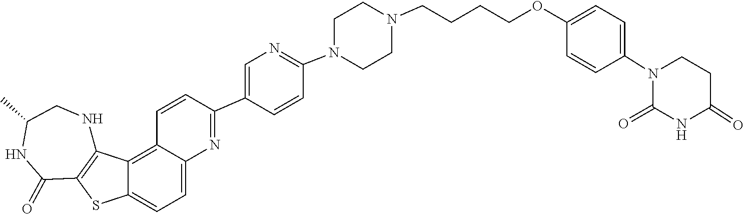 [SMILES: C[C@@H]1CNc2c(sc3ccc4nc(-c5ccc(N6CCN(CCCCOc7ccc(N8CCC(=O)NC8=O)cc7)CC6)nc5)ccc4c23)C(=O)N1] |
| I-438 | 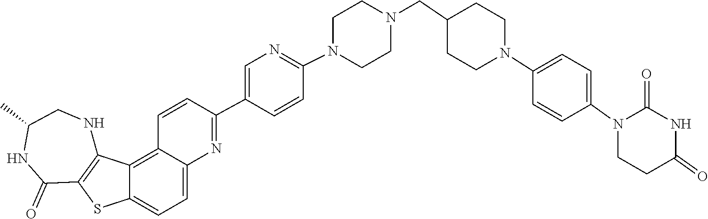 [SMILES: C[C@@H]1CNc2c(sc3ccc4nc(-c5ccc(N6CCN(CC7CCN(c8ccc(N9CCC(=O)NC9=O)cc8)CC7)CC6)nc5)ccc4c23)C(=O)N1] |
| I-439 | 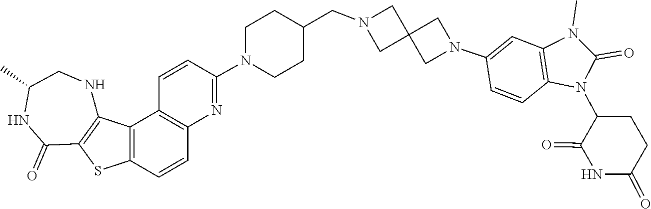 [SMILES: C[C@@H]1CNc2c(sc3ccc4nc(N5CCC(CN6CC7(C6)CN(c6ccc8c(c6)n(C)c(=O)n8C6CCC(=O)NC6=O)C7)CC5)ccc4c23)C(=O)N1] |
| I-440 | 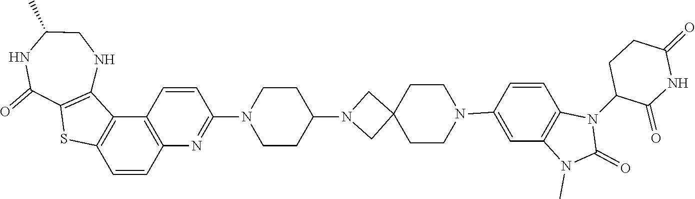 [SMILES: C[C@@H]1CNc2c(sc3ccc4nc(N5CCC(N6CC7(CCN(c8ccc9c(c8)n(C)c(=O)n9C8CCC(=O)NC8=O)CC7)C6)CC5)ccc4c23)C(=O)N1] |
| I-441 | 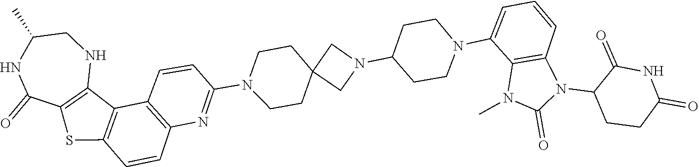 [SMILES: C[C@@H]1CNc2c(sc3ccc4nc(N5CCC6(CC5)CN(C5CCN(c7cccc8c7n(C)c(=O)n8C7CCC(=O)NC7=O)CC5)C6)ccc4c23)C(=O)N1] |
| I-442 | 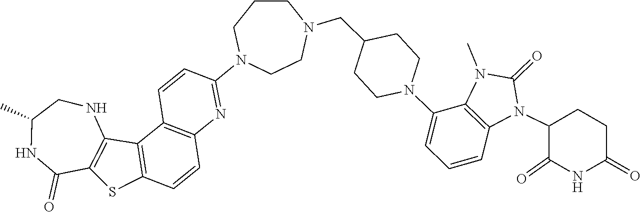 [SMILES: C[C@@H]1CNc2c(sc3ccc4nc(N5CCCN(CC6CCN(c7cccc8c7n(C)c(=O)n8C7CCC(=O)NC7=O)CC6)CC5)ccc4c23)C(=O)N1] |
| I-443 | 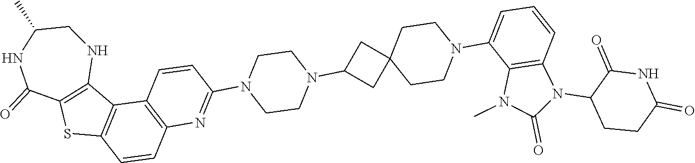 [SMILES: C[C@@H]1CNc2c(sc3ccc4nc(N5CCN(C6CC7(CCN(c8cccc9c8n(C)c(=O)n9C8CCC(=O)NC8=O)CC7)C6)CC5)ccc4c23)C(=O)N1] |
| I-444 | 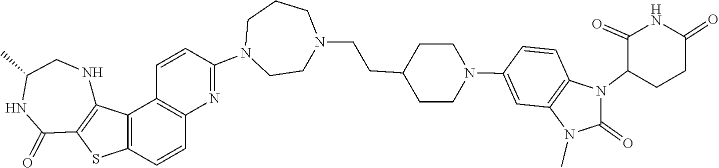 [SMILES: C[C@@H]1CNc2c(sc3ccc4nc(N5CCCN(CCC6CCN(c7ccc8c(c7)n(C)c(=O)n8C7CCC(=O)NC7=O)CC6)CC5)ccc4c23)C(=O)N1] |
| I-445 | 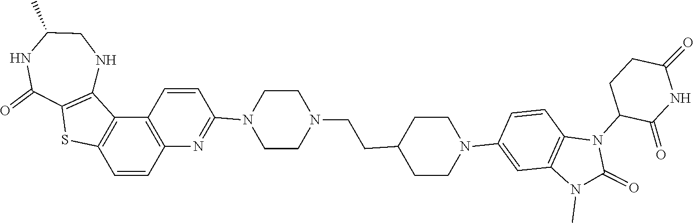 [SMILES: C[C@@H]1CNc2c(sc3ccc4nc(N5CCN(CCC6CCN(c7ccc8c(c7)n(C)c(=O)n8C7CCC(=O)NC7=O)CC6)CC5)ccc4c23)C(=O)N1] |
| I-446 | 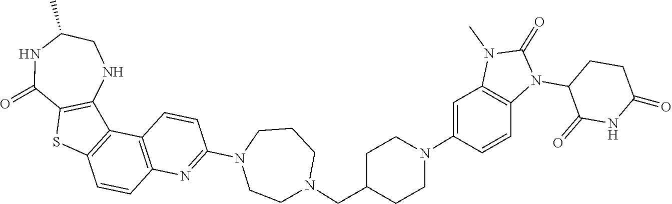 [SMILES: C[C@@H]1CNc2c(sc3ccc4nc(N5CCCN(CC6CCN(c7ccc8c(c7)n(C)c(=O)n8C7CCC(=O)NC7=O)CC6)CC5)ccc4c23)C(=O)N1] |
| I-447 | 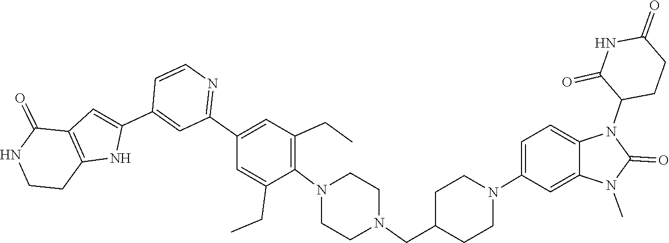 [SMILES: CCc1cc(-c2cc(-c3cc4c([nH]3)CCNC4=O)ccn2)cc(CC)c1N1CCN(CC2CCN(c3ccc4c(c3)n(C)c(=O)n4C3CCC(=O)NC3=O)CC2)CC1] |
| I-448 | 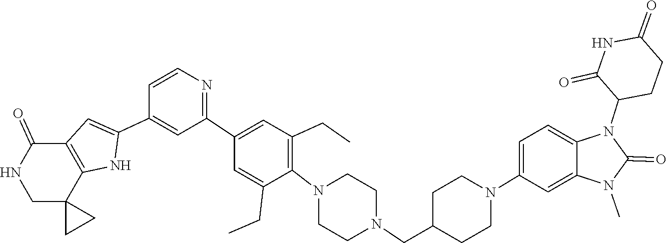 [SMILES: CCc1cc(-c2cc(-c3cc4c([nH]3)C3(CC3)CNC4=O)ccn2)cc(CC)c1N1CCN(CC2CCN(c3ccc4c(c3)n(C)c(=O)n4C3CCC(=O)NC3=O)CC2)CC1] |
| I-449 | 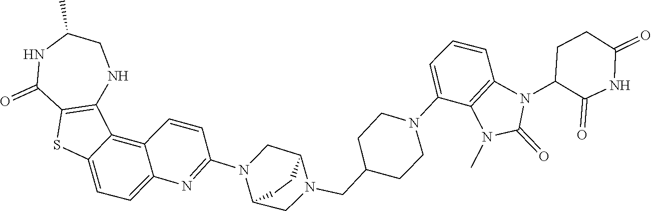 [SMILES: C[C@@H]1CNc2c(sc3ccc4nc(N5C[C@@H]6CC[C@H]5CCN6CC5CCN(c6cccc7c6n(C)c(=O)n7C6CCC(=O)NC6=O)CC5)ccc4c23)C(=O)N1] |
| I-450 | 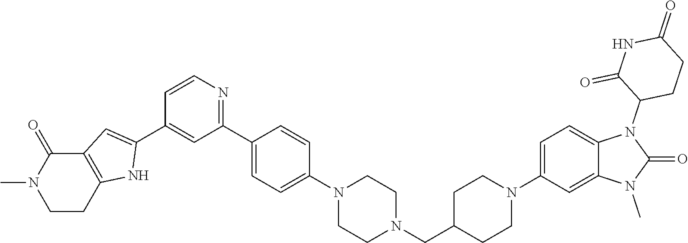 [SMILES: CN1CCc2[nH]c(-c3ccnc(-c4ccc(N5CCN(CC6CCN(c7ccc8c(c7)n(C)c(=O)n8C7CCC(=O)NC7=O)CC6)CC5)cc4)c3)cc2C1=O] |
| I-451 | 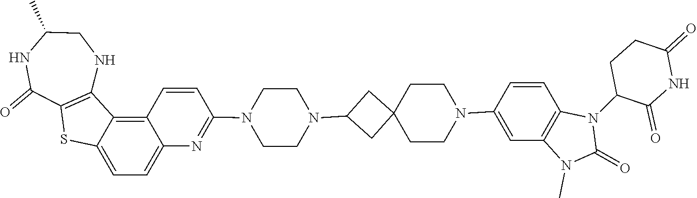 [SMILES: C[C@@H]1CNc2c(sc3ccc4nc(N5CCN(C6CC7(CCN(c8ccc9c(c8)n(C)c(=O)n9C8CCC(=O)NC8=O)CC7)C6)CC5)ccc4c23)C(=O)N1] |
| I-452 | 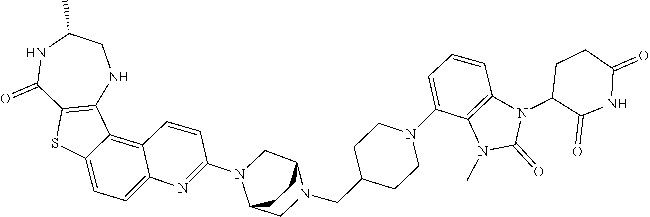 [SMILES: C[C@@H]1CNc2c(sc3ccc4nc(N5C[C@H]6CC[C@@H]5CN6CC5CCN(c6cccc7c6n(C)c(=O)n7C6CCC(=O)NC6=O)CC5)ccc4c23)C(=O)N1] |
| I-453 | 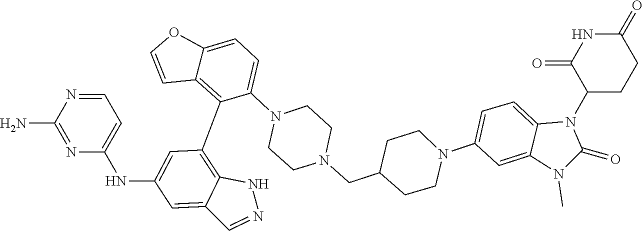 [SMILES: Cn1c(=O)n(C2CCC(=O)NC2=O)c2ccc(N3CCC(CN4CCN(c5ccc6occc6c5-c5cc(Nc6ccnc(N)n6)cc6cn[nH]c56)CC4)CC3)cc21] |
| I-454 | 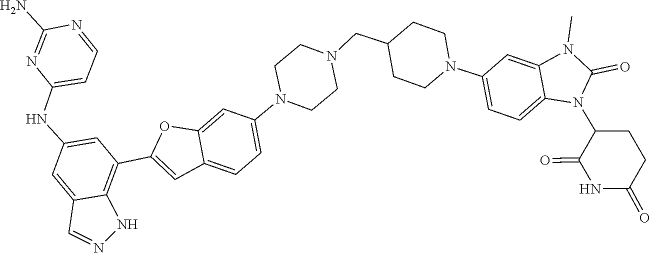 [SMILES: Cn1c(=O)n(C2CCC(=O)NC2=O)c2ccc(N3CCC(CN4CCN(c5ccc6cc(-c7cc(Nc8ccnc(N)n8)cc8cn[nH]c78)oc6c5)CC4)CC3)cc21] |
| I-458 | 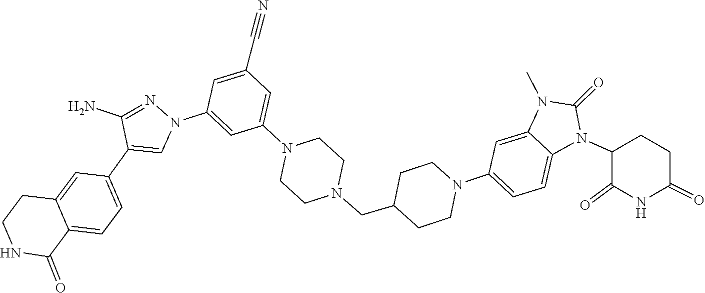 [SMILES: Cn1c(=O)n(C2CCC(=O)NC2=O)c2ccc(N3CCC(CN4CCN(c5cc(C#N)cc(-n6cc(-c7ccc8c(c7)CCNC8=O)c(N)n6)c5)CC4)CC3)cc21] |
| I-459 | 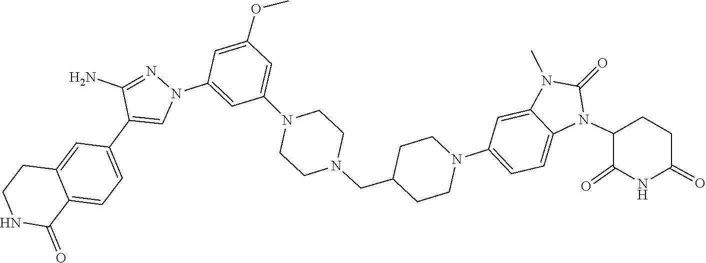 [SMILES: COc1cc(N2CCN(CC3CCN(c4ccc5c(c4)n(C)c(=O)n5C4CCC(=O)NC4=O)CC3)CC2)cc(-n2cc(-c3ccc4c(c3)CCNC4=O)c(N)n2)c1] |
| I-460 | 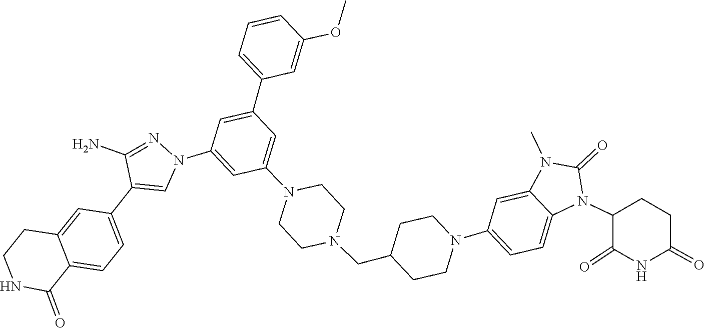 [SMILES: COc1cccc(-c2cc(N3CCN(CC4CCN(c5ccc6c(c5)n(C)c(=O)n6C5CCC(=O)NC5=O)CC4)CC3)cc(-n3cc(-c4ccc5c(c4)CCNC5=O)c(N)n3)c2)c1] |
| I-461 | 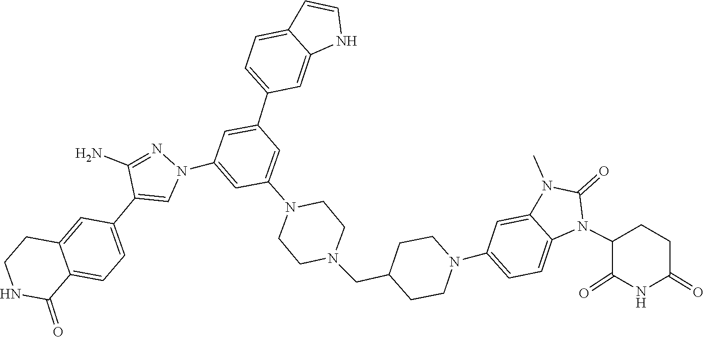 [SMILES: Cn1c(=O)n(C2CCC(=O)NC2=O)c2ccc(N3CCC(CN4CCN(c5cc(-c6ccc7cc[nH]c7c6)cc(-n6cc(-c7ccc8c(c7)CCNC8=O)c(N)n6)c5)CC4)CC3)cc21] |
| I-462 | 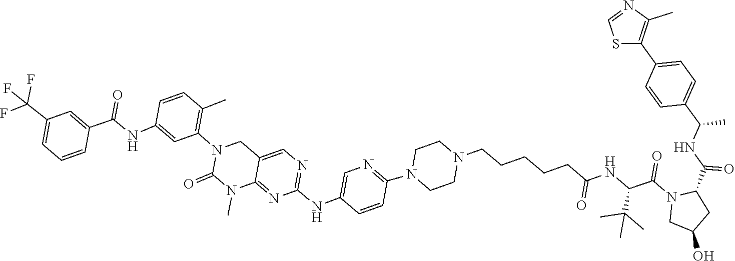 [SMILES: Cc1ccc(NC(=O)c2cccc(C(F)(F)F)c2)cc1N1Cc2cnc(Nc3ccc(N4CCN(CCCCCC(=O)N[C@H](C(=O)N5C[C@H](O)C[C@H]5C(=O)N[C@@H](C)c5ccc(-c6scnc6C)cc5)C(C)(C)C)CC4)nc3)nc2N(C)C1=O] |
| I-463 | 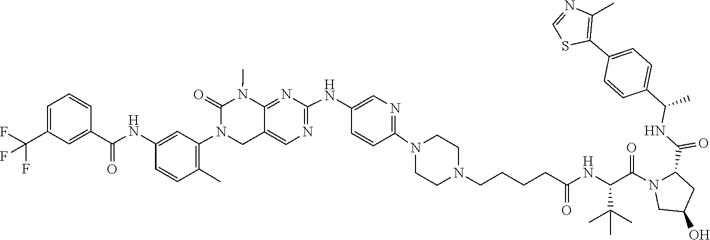 [SMILES: Cc1ccc(NC(=O)c2cccc(C(F)(F)F)c2)cc1N1Cc2cnc(Nc3ccc(N4CCN(CCCCC(=O)N[C@H](C(=O)N5C[C@H](O)C[C@H]5C(=O)N[C@@H](C)c5ccc(-c6scnc6C)cc5)C(C)(C)C)CC4)nc3)nc2N(C)C1=O] |
| I-464 | 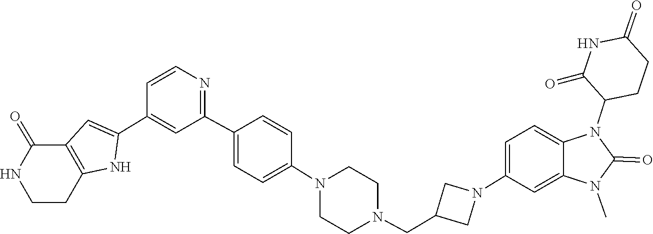 [SMILES: Cn1c(=O)n(C2CCC(=O)NC2=O)c2ccc(N3CC(CN4CCN(c5ccc(-c6cc(-c7cc8c([nH]7)CCNC8=O)ccn6)cc5)CC4)C3)cc21] |
| I-465 | 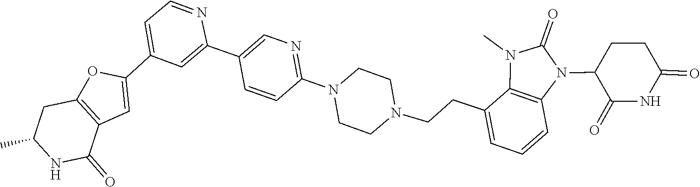 [SMILES: C[C@@H]1Cc2oc(-c3ccnc(-c4ccc(N5CCN(CCc6cccc7c6n(C)c(=O)n7C6CCC(=O)NC6=O)CC5)nc4)c3)cc2C(=O)N1] |
| I-466 | 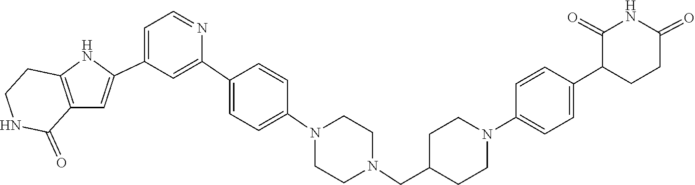 [SMILES: O=C1CCC(c2ccc(N3CCC(CN4CCN(c5ccc(-c6cc(-c7cc8c([nH]7)CCNC8=O)ccn6)cc5)CC4)CC3)cc2)C(=O)N1] |
| I-467 | 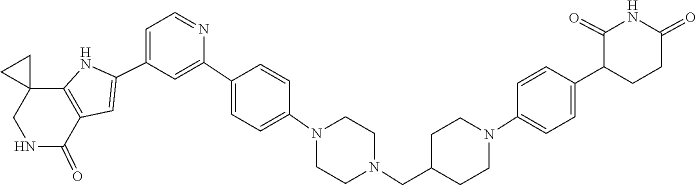 [SMILES: O=C1CCC(c2ccc(N3CCC(CN4CCN(c5ccc(-c6cc(-c7cc8c([nH]7)C7(CC7)CNC8=O)ccn6)cc5)CC4)CC3)cc2)C(=O)N1] |
| I-468 | 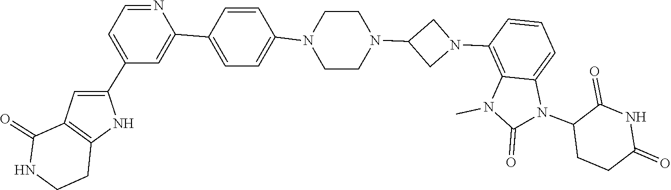 [SMILES: Cn1c(=O)n(C2CCC(=O)NC2=O)c2cccc(N3CC(N4CCN(c5ccc(-c6cc(-c7cc8c([nH]7)CCNC8=O)ccn6)cc5)CC4)C3)c21] |
| I-469 | 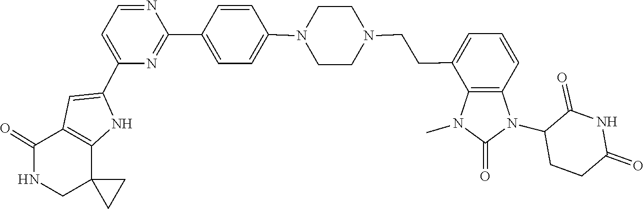 [SMILES: Cn1c(=O)n(C2CCC(=O)NC2=O)c2cccc(CCN3CCN(c4ccc(-c5nccc(-c6cc7c([nH]6)C6(CC6)CNC7=O)n5)cc4)CC3)c21] |
| I-470 | 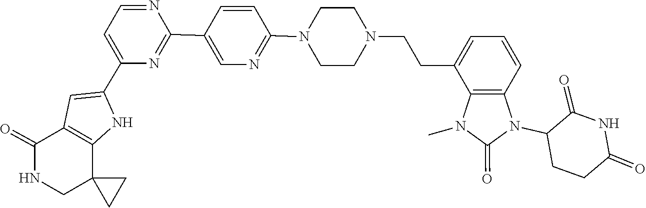 [SMILES: Cn1c(=O)n(C2CCC(=O)NC2=O)c2cccc(CCN3CCN(c4ccc(-c5nccc(-c6cc7c([nH]6)C6(CC6)CNC7=O)n5)cn4)CC3)c21] |
| I-471 | 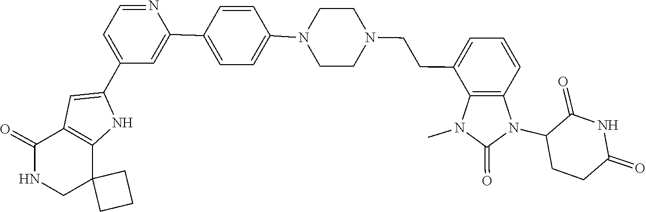 [SMILES: Cn1c(=O)n(C2CCC(=O)NC2=O)c2cccc(CCN3CCN(c4ccc(-c5cc(-c6cc7c([nH]6)C6(CCC6)CNC7=O)ccn5)cc4)CC3)c21] |
| I-472 | 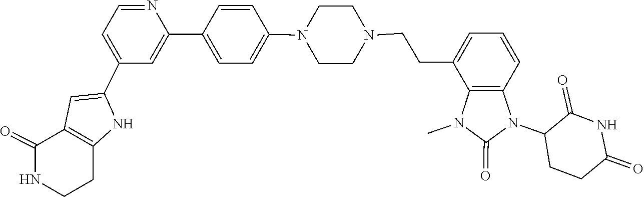 [SMILES: Cn1c(=O)n(C2CCC(=O)NC2=O)c2cccc(CCN3CCN(c4ccc(-c5cc(-c6cc7c([nH]6)CCNC7=O)ccn5)cc4)CC3)c21] |
| I-473 | 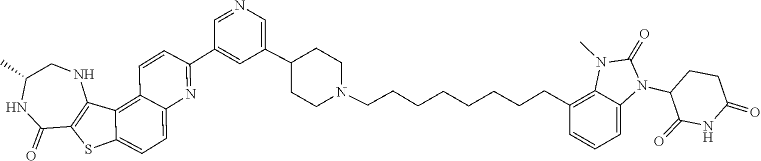 [SMILES: C[C@@H]1CNc2c(sc3ccc4nc(-c5cncc(C6CCN(CCCCCCCCc7cccc8c7n(C)c(=O)n8C7CCC(=O)NC7=O)CC6)c5)ccc4c23)C(=O)N1] |
| I-474 | 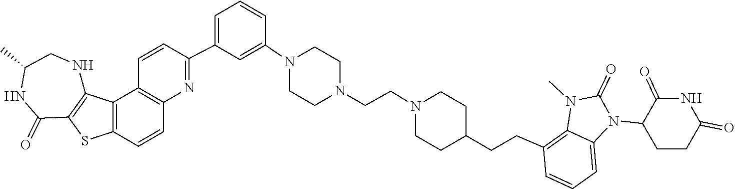 [SMILES: C[C@@H]1CNc2c(sc3ccc4nc(-c5cccc(N6CCN(CCN7CCC(CCc8cccc9c8n(C)c(=O)n9C8CCC(=O)NC8=O)CC7)CC6)c5)ccc4c23)C(=O)N1] |
| I-475 | 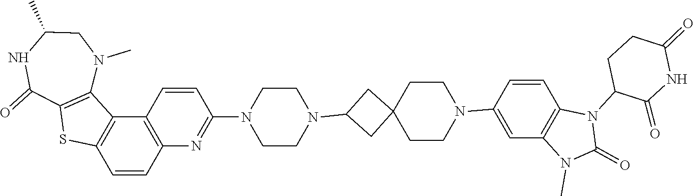 [SMILES: C[C@@H]1CN(C)c2c(sc3ccc4nc(N5CCN(C6CC7(CCN(c8ccc9c(c8)n(C)c(=O)n9C8CCC(=O)NC8=O)CC7)C6)CC5)ccc4c23)C(=O)N1] |
| I-476 | 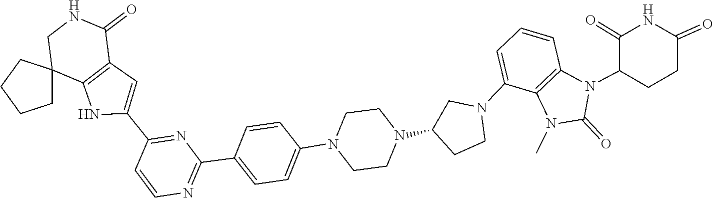 [SMILES: Cn1c(=O)n(C2CCC(=O)NC2=O)c2cccc(N3CC[C@H](N4CCN(c5ccc(-c6nccc(-c7cc8c([nH]7)C7(CCCC7)CNC8=O)n6)cc5)CC4)C3)c21] |
| I-477 | 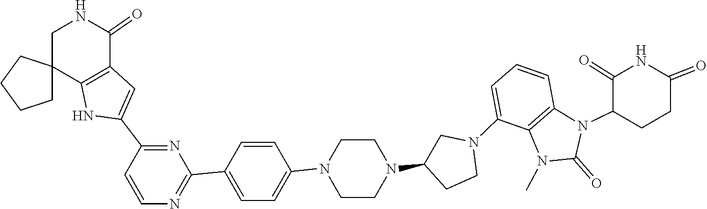 [SMILES: Cn1c(=O)n(C2CCC(=O)NC2=O)c2cccc(N3CC[C@@H](N4CCN(c5ccc(-c6nccc(-c7cc8c([nH]7)C7(CCCC7)CNC8=O)n6)cc5)CC4)C3)c21] |
| I-478 | 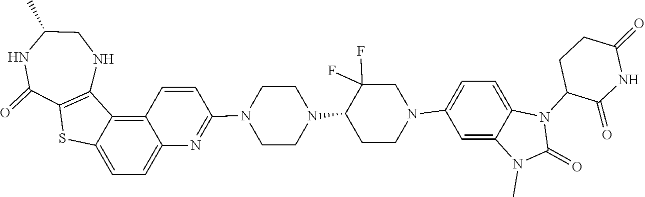 [SMILES: C[C@@H]1CNc2c(sc3ccc4nc(N5CCN([C@H]6CCN(c7ccc8c(c7)n(C)c(=O)n8C7CCC(=O)NC7=O)CC6(F)F)CC5)ccc4c23)C(=O)N1] |
| I-479 | 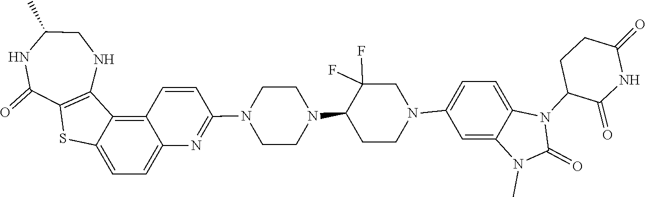 [SMILES: C[C@@H]1CNc2c(sc3ccc4nc(N5CCN([C@@H]6CCN(c7ccc8c(c7)n(C)c(=O)n8C7CCC(=O)NC7=O)CC6(F)F)CC5)ccc4c23)C(=O)N1] |
| I-480 | 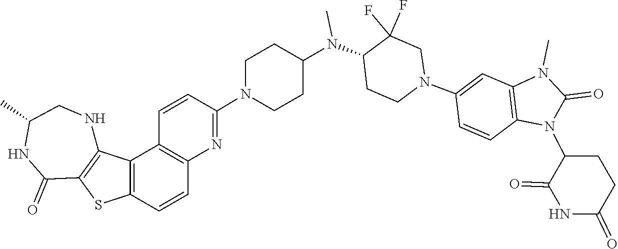 [SMILES: C[C@@H]1CNc2c(sc3ccc4nc(N5CCC(N(C)[C@H]6CCN(c7ccc8c(c7)n(C)c(=O)n8C7CCC(=O)NC7=O)CC6(F)F)CC5)ccc4c23)C(=O)N1] |
| I-481 | 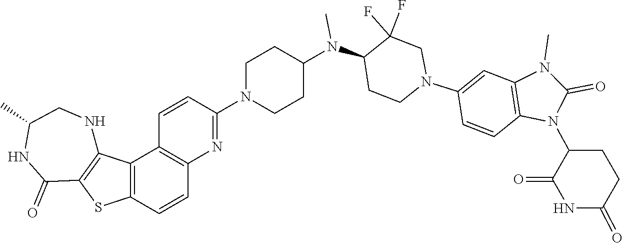 [SMILES: C[C@@H]1CNc2c(sc3ccc4nc(N5CCC(N(C)[C@@H]6CCN(c7ccc8c(c7)n(C)c(=O)n8C7CCC(=O)NC7=O)CC6(F)F)CC5)ccc4c23)C(=O)N1] |
| I-482 | 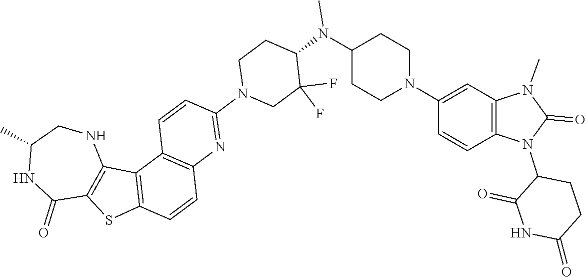 [SMILES: C[C@@H]1CNc2c(sc3ccc4nc(N5CC[C@H](N(C)C6CCN(c7ccc8c(c7)n(C)c(=O)n8C7CCC(=O)NC7=O)CC6)C(F)(F)C5)ccc4c23)C(=O)N1] |
| I-483 | 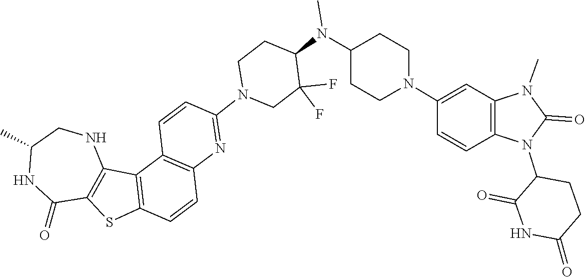 [SMILES: C[C@@H]1CNc2c(sc3ccc4nc(N5CC[C@@H](N(C)C6CCN(c7ccc8c(c7)n(C)c(=O)n8C7CCC(=O)NC7=O)CC6)C(F)(F)C5)ccc4c23)C(=O)N1] |
| I-484 | 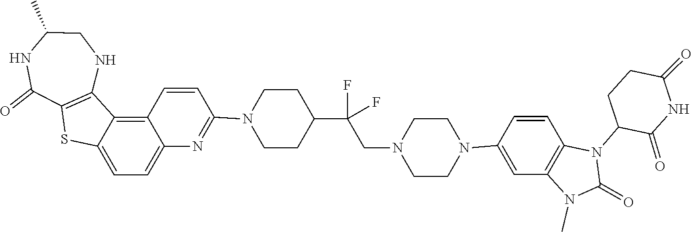 [SMILES: C[C@@H]1CNc2c(sc3ccc4nc(N5CCC(C(F)(F)CN6CCN(c7ccc8c(c7)n(C)c(=O)n8C7CCC(=O)NC7=O)CC6)CC5)ccc4c23)C(=O)N1] |
| I-485 | 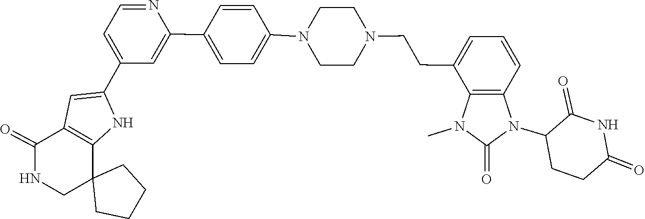 [SMILES: Cn1c(=O)n(C2CCC(=O)NC2=O)c2cccc(CCN3CCN(c4ccc(-c5cc(-c6cc7c([nH]6)C6(CCCC6)CNC7=O)ccn5)cc4)CC3)c21] |
| I-486 | 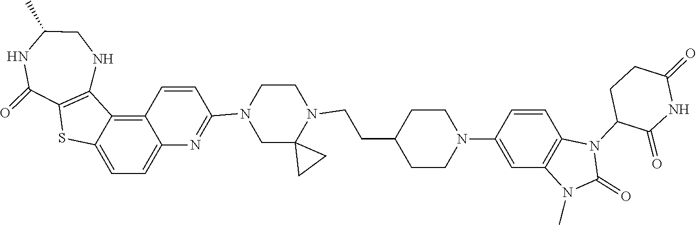 [SMILES: C[C@@H]1CNc2c(sc3ccc4nc(N5CCN(CCC6CCN(c7ccc8c(c7)n(C)c(=O)n8C7CCC(=O)NC7=O)CC6)C6(CC6)C5)ccc4c23)C(=O)N1] |
| I-487 | 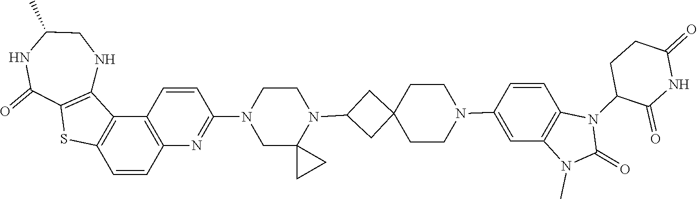 [SMILES: C[C@@H]1CNc2c(sc3ccc4nc(N5CCN(C6CC7(CCN(c8ccc9c(c8)n(C)c(=O)n9C8CCC(=O)NC8=O)CC7)C6)C6(CC6)C5)ccc4c23)C(=O)N1] |
| I-488 | 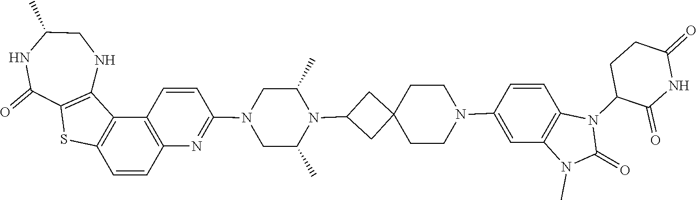 [SMILES: C[C@@H]1CNc2c(sc3ccc4nc(N5C[C@@H](C)N(C6CC7(CCN(c8ccc9c(c8)n(C)c(=O)n9C8CCC(=O)NC8=O)CC7)C6)[C@@H](C)C5)ccc4c23)C(=O)N1] |
| I-489 | 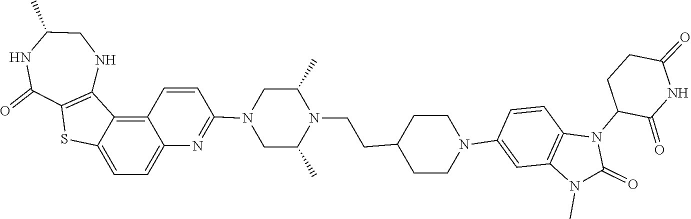 [SMILES: C[C@@H]1CNc2c(sc3ccc4nc(N5C[C@@H](C)N(CCC6CCN(c7ccc8c(c7)n(C)c(=O)n8C7CCC(=O)NC7=O)CC6)[C@@H](C)C5)ccc4c23)C(=O)N1] |
| I-490 | 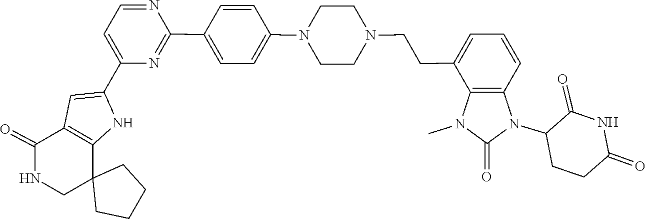 [SMILES: Cn1c(=O)n(C2CCC(=O)NC2=O)c2cccc(CCN3CCN(c4ccc(-c5nccc(-c6cc7c([nH]6)C6(CCCC6)CNC7=O)n5)cc4)CC3)c21] |
| I-491 | 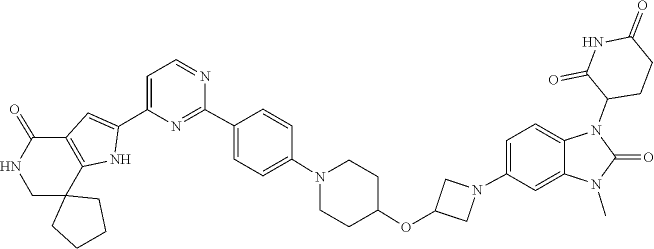 [SMILES: Cn1c(=O)n(C2CCC(=O)NC2=O)c2ccc(N3CC(OC4CCN(c5ccc(-c6nccc(-c7cc8c([nH]7)C7(CCCC7)CNC8=O)n6)cc5)CC4)C3)cc21] |
| I-492 | 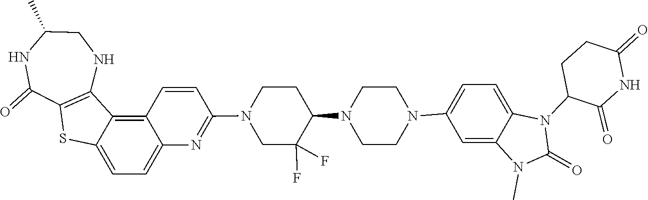 [SMILES: C[C@@H]1CNc2c(sc3ccc4nc(N5CC[C@@H](N6CCN(c7ccc8c(c7)n(C)c(=O)n8C7CCC(=O)NC7=O)CC6)C(F)(F)C5)ccc4c23)C(=O)N1] |
| I-493 | 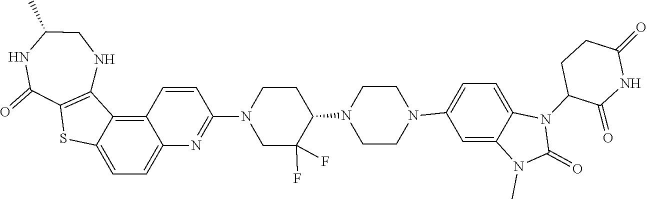 [SMILES: C[C@@H]1CNc2c(sc3ccc4nc(N5CC[C@H](N6CCN(c7ccc8c(c7)n(C)c(=O)n8C7CCC(=O)NC7=O)CC6)C(F)(F)C5)ccc4c23)C(=O)N1] |
| I-494 | 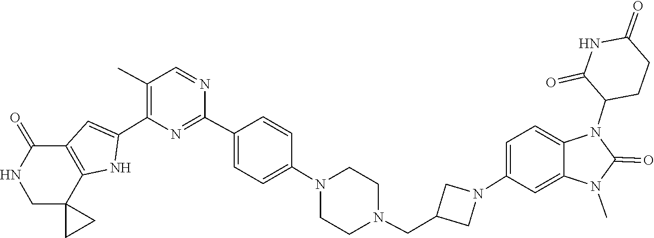 [SMILES: Cc1cnc(-c2ccc(N3CCN(CC4CN(c5ccc6c(c5)n(C)c(=O)n6C5CCC(=O)NC5=O)C4)CC3)cc2)nc1-c1cc2c([nH]1)C1(CC1)CNC2=O] |
| I-495 | 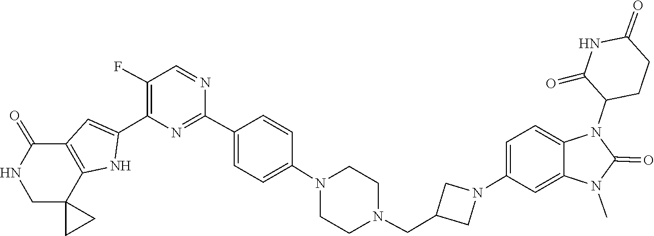 [SMILES: Cn1c(=O)n(C2CCC(=O)NC2=O)c2ccc(N3CC(CN4CCN(c5ccc(-c6ncc(F)c(-c7cc8c([nH]7)C7(CC7)CNC8=O)n6)cc5)CC4)C3)cc21] |
| I-496 | 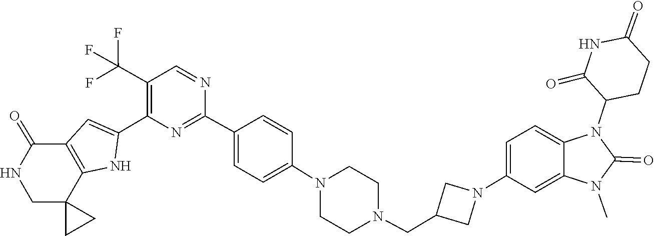 [SMILES: Cn1c(=O)n(C2CCC(=O)NC2=O)c2ccc(N3CC(CN4CCN(c5ccc(-c6ncc(C(F)(F)F)c(-c7cc8c([nH]7)C7(CC7)CNC8=O)n6)cc5)CC4)C3)cc21] |
| I-497 | 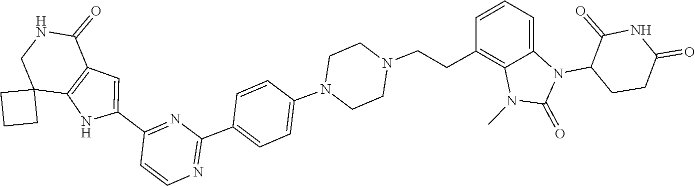 [SMILES: Cn1c(=O)n(C2CCC(=O)NC2=O)c2cccc(CCN3CCN(c4ccc(-c5nccc(-c6cc7c([nH]6)C6(CCC6)CNC7=O)n5)cc4)CC3)c21] |
| I-498 | 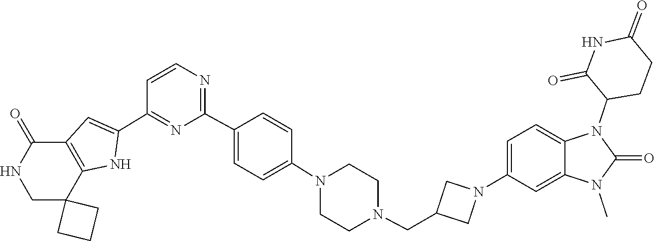 [SMILES: Cn1c(=O)n(C2CCC(=O)NC2=O)c2ccc(N3CC(CN4CCN(c5ccc(-c6nccc(-c7cc8c([nH]7)C7(CCC7)CNC8=O)n6)cc5)CC4)C3)cc21] |
| I-499 | 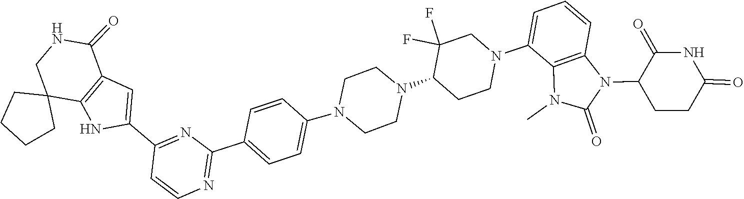 [SMILES: Cn1c(=O)n(C2CCC(=O)NC2=O)c2cccc(N3CC[C@H](N4CCN(c5ccc(-c6nccc(-c7cc8c([nH]7)C7(CCCC7)CNC8=O)n6)cc5)CC4)C(F)(F)C3)c21] |
| I-500 | 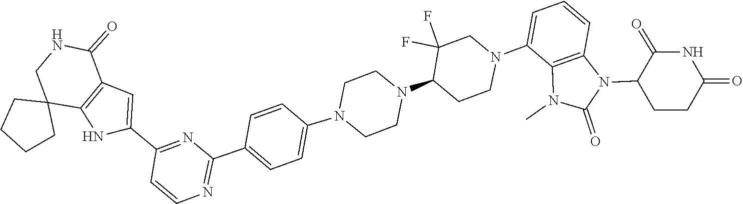 [SMILES: Cn1c(=O)n(C2CCC(=O)NC2=O)c2cccc(N3CC[C@@H](N4CCN(c5ccc(-c6nccc(-c7cc8c([nH]7)C7(CCCC7)CNC8=O)n6)cc5)CC4)C(F)(F)C3)c21] |
| I-501 | 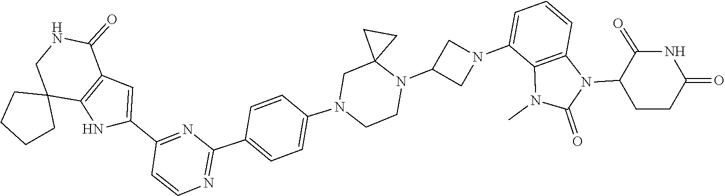 [SMILES: Cn1c(=O)n(C2CCC(=O)NC2=O)c2cccc(N3CC(N4CCN(c5ccc(-c6nccc(-c7cc8c([nH]7)C7(CCCC7)CNC8=O)n6)cc5)CC45CC5)C3)c21] |
| I-502 | 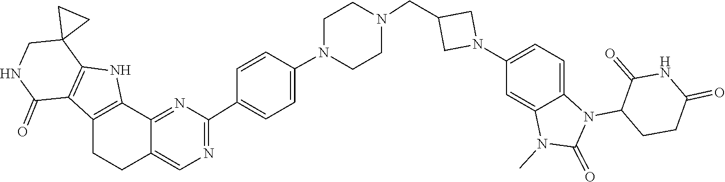 [SMILES: Cn1c(=O)n(C2CCC(=O)NC2=O)c2ccc(N3CC(CN4CCN(c5ccc(-c6ncc7c(n6)-c6[nH]c8c(c6CC7)C(=O)NCC86CC6)cc5)CC4)C3)cc21] |
| I-503 | 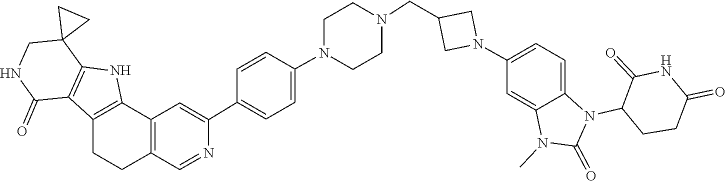 [SMILES: Cn1c(=O)n(C2CCC(=O)NC2=O)c2ccc(N3CC(CN4CCN(c5ccc(-c6cc7c(cn6)CCc6c-7[nH]c7c6C(=O)NCC76CC6)cc5)CC4)C3)cc21] |
| I-504 | 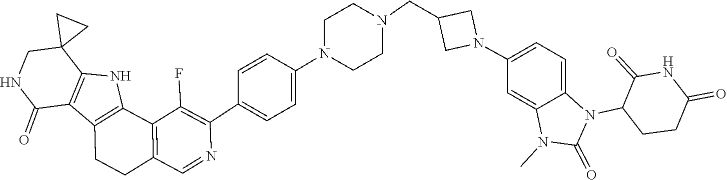 [SMILES: Cn1c(=O)n(C2CCC(=O)NC2=O)c2ccc(N3CC(CN4CCN(c5ccc(-c6ncc7c(c6F)-c6[nH]c8c(c6CC7)C(=O)NCC86CC6)cc5)CC4)C3)cc21] |
| I-505 | 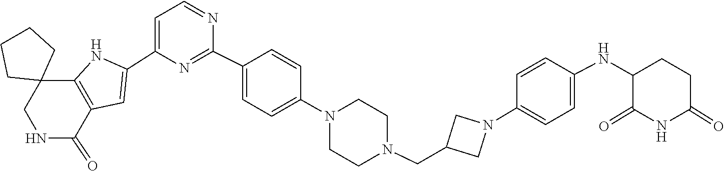 [SMILES: O=C1CCC(Nc2ccc(N3CC(CN4CCN(c5ccc(-c6nccc(-c7cc8c([nH]7)C7(CCCC7)CNC8=O)n6)cc5)CC4)C3)cc2)C(=O)N1] |
| I-506 | 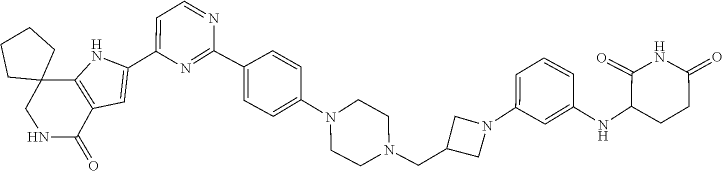 [SMILES: O=C1CCC(Nc2cccc(N3CC(CN4CCN(c5ccc(-c6nccc(-c7cc8c([nH]7)C7(CCCC7)CNC8=O)n6)cc5)CC4)C3)c2)C(=O)N1] |
| I-507 | 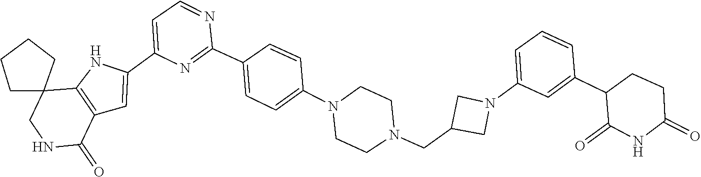 [SMILES: O=C1CCC(c2cccc(N3CC(CN4CCN(c5ccc(-c6nccc(-c7cc8c([nH]7)C7(CCCC7)CNC8=O)n6)cc5)CC4)C3)c2)C(=O)N1] |
| I-508 | 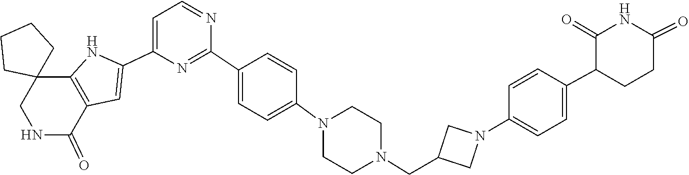 [SMILES: O=C1CCC(c2ccc(N3CC(CN4CCN(c5ccc(-c6nccc(-c7cc8c([nH]7)C7(CCCC7)CNC8=O)n6)cc5)CC4)C3)cc2)C(=O)N1] |
| I-509 | 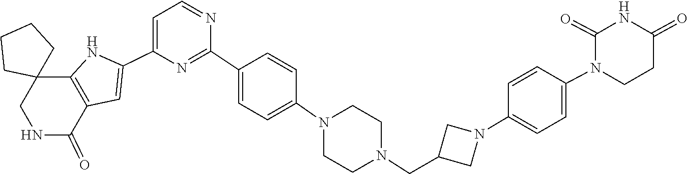 [SMILES: O=C1CCN(c2ccc(N3CC(CN4CCN(c5ccc(-c6nccc(-c7cc8c([nH]7)C7(CCCC7)CNC8=O)n6)cc5)CC4)C3)cc2)C(=O)N1] |
| I-510 | 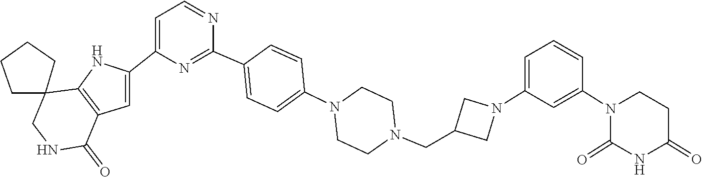 [SMILES: O=C1CCN(c2cccc(N3CC(CN4CCN(c5ccc(-c6nccc(-c7cc8c([nH]7)C7(CCCC7)CNC8=O)n6)cc5)CC4)C3)c2)C(=O)N1] |
| I-511 | 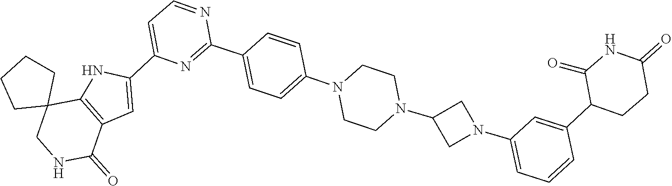 [SMILES: O=C1CCC(c2cccc(N3CC(N4CCN(c5ccc(-c6nccc(-c7cc8c([nH]7)C7(CCCC7)CNC8=O)n6)cc5)CC4)C3)c2)C(=O)N1] |
| I-512 | 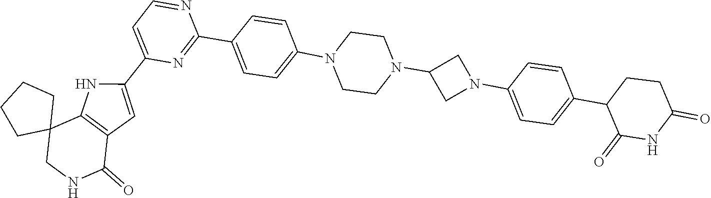 [SMILES: O=C1CCC(c2ccc(N3CC(N4CCN(c5ccc(-c6nccc(-c7cc8c([nH]7)C7(CCCC7)CNC8=O)n6)cc5)CC4)C3)cc2)C(=O)N1] |
| I-513 | 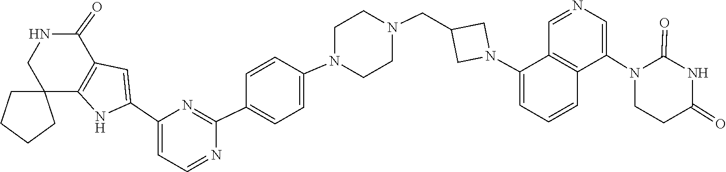 [SMILES: O=C1CCN(c2cncc3c(N4CC(CN5CCN(c6ccc(-c7nccc(-c8cc9c([nH]8)C8(CCCC8)CNC9=O)n7)cc6)CC5)C4)cccc23)C(=O)N1] |
| I-514 | 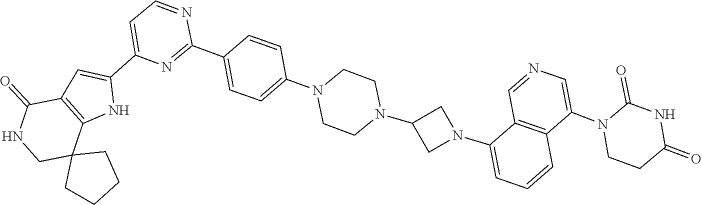 [SMILES: O=C1CCN(c2cncc3c(N4CC(N5CCN(c6ccc(-c7nccc(-c8cc9c([nH]8)C8(CCCC8)CNC9=O)n7)cc6)CC5)C4)cccc23)C(=O)N1] |
| I-515 | 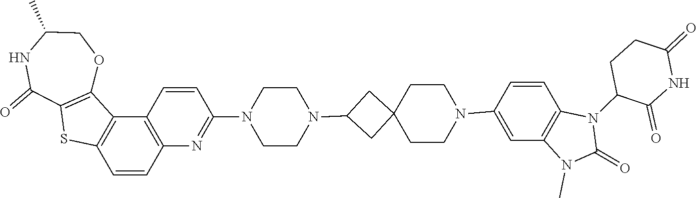 [SMILES: C[C@@H]1COc2c(sc3ccc4nc(N5CCN(C6CC7(CCN(c8ccc9c(c8)n(C)c(=O)n9C8CCC(=O)NC8=O)CC7)C6)CC5)ccc4c23)C(=O)N1] |
| I-516 | 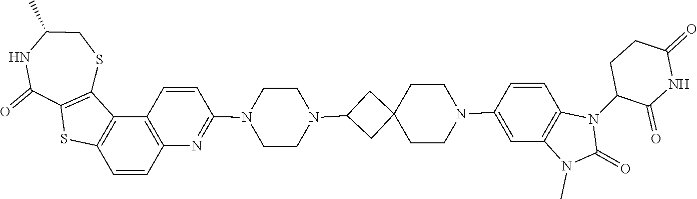 [SMILES: C[C@@H]1CSc2c(sc3ccc4nc(N5CCN(C6CC7(CCN(c8ccc9c(c8)n(C)c(=O)n9C8CCC(=O)NC8=O)CC7)C6)CC5)ccc4c23)C(=O)N1] |
| I-517 | 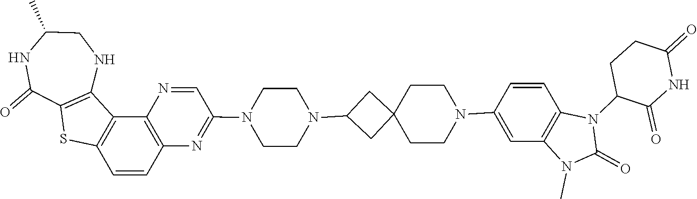 [SMILES: C[C@@H]1CNc2c(sc3ccc4nc(N5CCN(C6CC7(CCN(c8ccc9c(c8)n(C)c(=O)n9C8CCC(=O)NC8=O)CC7)C6)CC5)cnc4c23)C(=O)N1] |

In some embodiments, the present invention provides a compound set forth in Table 1, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below, where a provided compound is formed having a reactive moiety (e.g., amine, alcohol, etc.), it is not shown but it is generally appreciated and well known by those having ordinary skill in the art that the reactivity of said reactive moiety may be masked by employing a suitable protecting group that can thereafter be removed in situ or during a separate synthetic step.

In certain embodiments, compounds of the present invention are generally prepared according to Scheme 1 set forth below:

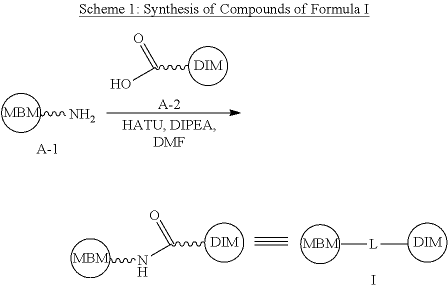

As depicted in Scheme 1, above, amine A-1 is coupled to acid A-2 using the coupling agent HATU in the presence of the base DIPEA in DMF to form a compound of formula I with a linker comprising an amide bond. The squiggly bond, [symbol] represents the portion of the linker between MBM and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 2 set forth below:

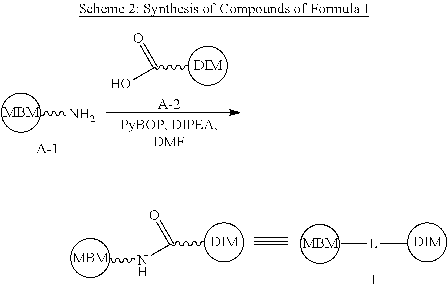

As depicted in Scheme 2, above, amine A-1 is coupled to acid A-2 using the coupling agent PyBOP in the presence of the base DIPEA in DMF to form a compound of formula I with a linker comprising an amide bond. The squiggly bond, [symbol] , represents the portion of the linker between MBM and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 3 set forth below:

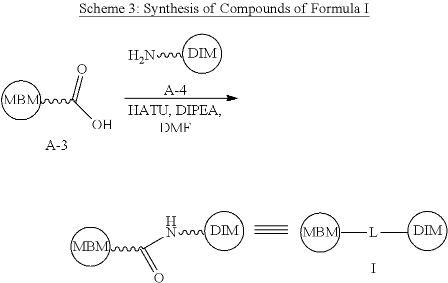

As depicted in Scheme 3, above, acid A-3 is coupled to amine A-4 using the coupling agent HATU in the presence of the base DIPEA in DMF to form a compound of formula I with a linker comprising an amide bond. The squiggly bond, [symbol] , represents the portion of the linker between MBM and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 4 set forth below:

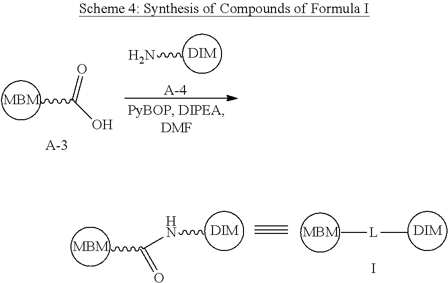

As depicted in Scheme 4, above, acid A-3 is coupled to amine A-4 using the coupling agent PyBOP in the presence of the base DIPEA in DMF to form a compound of formula I with a linker comprising an amide bond. The squiggly bond, [symbol] , represents the portion of the linker between MBM and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 5 set forth below:

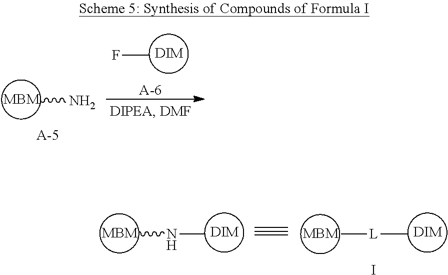

As depicted in Scheme 5, above, an S_{N}Ar displacement of fluoride A-6 by amine A-5 is effected in the presence of the base DIPEA in DMF to form a compound of formula I with a linker comprising a secondary amine. The squiggly bond, [symbol] , represents the portion of the linker between MBM and the terminal amino group of A-5.

In certain embodiments, compounds of the present invention are generally prepared according to Scheme 6 set forth below:

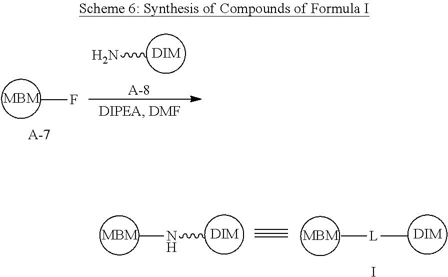

As depicted in Scheme 6, above, an S_{N}Ar displacement of fluoride A-7 by amine A-8 is effected in the presence of the base DIPEA in DMF to form a compound of formula I with a linker comprising a secondary amine. The squiggly bond, [symbol] , represents the portion of the linker between DIM and the terminal amino group of A-8.

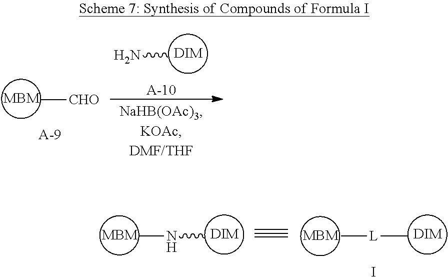

As depicted in Scheme 7, above, reductive amination of the mixture of aldehyde A-9 and amine A-10 is effected in the presence of NaHB(OAc)₃ and KOAc in DMF/THF to form a compound of formula I with a linker comprising a secondary amine. The squiggly bond, [symbol] , represents the portion of the linker between DIM and the terminal amino group of A-8.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the invention are described below in the Exemplification.

### 5. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably degrade and/or inhibit a MK2 protein, or a mutant thereof, in a biological sample or in a patient. In certain embodiments, the amount of compound in compositions of this invention is such that is effective to measurably degrade and/or inhibit an MK2 protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitorily active metabolite or residue thereof” means that a metabolite or residue thereof is also an inhibitor of a MK2 protein, or a mutant thereof.

As used herein, the term “degratorily active metabolite or residue thereof” means that a metabolite or residue thereof is also a degrader of an MK2 protein, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Compounds and compositions described herein are generally useful for the degradation and/or inhibition of MK2 protein activity.

The method of the present invention is useful for, but not limited to, the prevention and/or treatment of diseases and disorders that are mediated by pro-inflammatory molecules such as TNFα, IL-1, and IL-6, including pain, inflammation, tissue damage, and/or arthritis. For example, the compounds described herein would be useful for the treatment of any inflammation-related disorder described below, such as an analgesic in the treatment of pain and headaches, or as an antipyretic for the treatment of fever. The compounds described herein would also be useful for the treatment of an inflammation-related disorder in a subject suffering from such an inflammation-associated disorder. In preferred embodiments, the methods and compositions of the present invention encompass the prevention and/or treatment of autoimmune and inflammation-related disorders.

As used herein, the terms “MK2-mediated” disorders, diseases, and/or conditions as used herein means any disease or other deleterious condition in which one or more MK2, or a mutant thereof, are known to play a role. Accordingly, another embodiment of the present invention relates to treating or lessening the severity of one or more diseases in which MK2, or a mutant thereof, are known to play a role.

As used herein, the terms “TNFα, IL-1, or IL-6 mediated disease or disorder” are meant to include, without limitation, each of the symptoms or diseases that is mentioned herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

In some embodiments, the methods and compositions of the present invention encompass the treatment of any one or more of the disorders selected from a connective tissue and joint disorders, neoplasia disorders, cardiovascular disorders, otic disorders, ophthalmic disorders, respiratory disorders, gastrointestinal disorders, angiogenesis-related disorders, immunological disorders, inflammatory disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

As used herein, the terms “neoplasia” and “neoplasia disorder”, used interchangeably herein, refer to new cell growth that results from a loss of responsiveness to normal growth controls, e.g. to “neoplastic” cell growth. Neoplasia is also used interchangeably herein with the term “cancer” and for purposes of the present invention; cancer is one subtype of neoplasia. As used herein, the term “neoplasia disorder” also encompasses other cellular abnormalities, such as hyperplasia, metaplasia and dysplasia. The terms neoplasia, metaplasia, dysplasia and hyperplasia can be used interchangeably herein and refer generally to cells experiencing abnormal cell growth. Both of the terms, “neoplasia” and “neoplasia disorder”, refer to a “neoplasm” or tumor, which may be benign, premalignant, metastatic, or malignant. Also encompassed by the present invention are benign, premalignant, metastatic, or malignant neoplasias. Also encompassed by the present invention are benign, premalignant, metastatic, or malignant tumors. Thus, all of benign, premalignant, metastatic, or malignant neoplasia or tumors are encompassed by the present invention and may be referred to interchangeably, as neoplasia, neoplasms or neoplasia-related disorders. Tumors are generally known in the art to be a mass of neoplasia or “neoplastic” cells. Although, it is to be understood that even one neoplastic cell is considered, for purposes of the present invention to be a neoplasm or alternatively, neoplasia.

In some embodiments, the MK2-mediated disease or disorder is chosen from a skin disorder, pruritus, a hair loss disorder, a cancer, a neoplasm, Alzheimer's disease, an inflammatory condition, connective tissue diseases and an autoimmune condition.

In certain embodiments, the MK2-mediated disease or disorder is a neoplasm, a malignancy, a myeloproliferative disorder, a hematopoietic neoplasm, a myeloid neoplasm, a lymphoid neoplasm, including myelofibrosis, primary myelofibrosis, polycythemia vera, essential thrombocythemia, acute and chronic leukemias, lymphomas, cutaneous lymphomas including mycosis fungoides, other myeloid malignancies, and myelodysplastic syndrome.

In certain embodiments, the methods described herein are used to treat patients with autoimmune disorders or responses, broad activation of the immune responses, bacterial infection, viral infection, inflammation, a chronic and/or acute inflammatory disorder or condition, and/or autoinflammatory disorder, rheumatic diseases, fibrotic disorders, metabolic disorders, a neoplasm, or cardiovascular or cerebrovascular disorders, a skin disorder, pruritus, a hair loss disorder, a cancer or malignancy, autoimmune connective tissue diseases and an autoimmune condition; Still's disease, adult-onset Still's disease, Th17-associated inflammation, polychondritis (e.g., relapsing polychondritis); myositis, polymyositis, autoimmune myositis, dermatomyositis, juvenile dermatomyositis; myasthenia gravis; Arthritis (e.g., rheumatoid arthritis, juvenile rheumatoid arthritis, systemic-onset juvenile rheumatoid arthritis, osteoarthritis, infectious arthritis, inflammatory arthritis, inflammatory bowel disease-associated arthritis, idiopathic arthritis, juvenile idiopathic arthritis, systemic juvenile idiopathic arthritis, psoriatic arthritis), spondylitis/spondyloarthritis/spondyloarthropathy (ankylosing spondylitis), gout, scleroderma (systemic scleroderma, juvenile scleroderma), Reiter's syndrome/reactive arthritis, Lyme disease, lupus/systemic lupus erythematosus (SLE) (lupus erythematosus, pediatric systemic lupus erythematosus, cutaneous lupus (subacute cutaneous lupus, chronic cutaneous lupus/discoid lupus, chilblain lupus erythematosus), polymyalgia rheumatica, enthesitis, mixed connective tissue disease, enthesopathy; carditis, myocarditis, angiogenesis disorders, myelodysplastic syndrome, atherosclerosis, atherosclerosis and hypercholesterolemia, restenosis (restenosis of an atherosclerotic coronary artery), acute coronary syndrome, myocardial infarction, cardiac-allograft vasculopathy, transplant arteriopathy; vasculitis (large vessel vasculitis, small vessel vasculitis, giant-cell arteritis, polyarteritis nodosa, vasculitis syndromes including: Takayasu's arteritis, Wegener's granulomatosis, Behcet's Disease), stimulator of interferon genes (STING) associated vasculopathy with onset in infancy (SAVI); gastrointestinal disorders, enterocolitis, colitis, inflammatory bowel disease (ulcerative colitis, Crohn's disease), irritable bowel syndrome, enteritis syndrome/spastic colon, celiac disease; acute and chronic pancreatitis; primary biliary cirrhosis, primary sclerosing cholangitis, jaundice, cirrhosis (for example, primary biliary cirrhosis or cirrhosis due to fatty liver disease (for example, alcoholic and nonalcoholic steatosis); esophagitis, gastritis, gastric and duodenal ulcers, peritonitis; Nephropathies: immunologically mediated glomerulonephropathy, autoimmune nephropathy, membranous glomerulopathy, chronic progressive nephropathies, diabetic kidney disease/diabetic nephropathy, renal fibrosis, renal ischemic/reperfusion injury, HIV associated nephropathy, ureteral obstructive nephropathy, glomerulosclerosis, proteinuria, nephrotic syndrome, polycystic kidney disease, autosomal dominant polycystic kidney disease, a nephropathy is an immunologically mediated nephropathy, autoimmune nephropathy, chronic progressive nephropathies, diabetic nephropathy, renal fibrosis, ischemic/reperfusion injury associated, HIV associated nephropathy, ureteral obstructive nephropathy, glomerulonephritis, chronic kidney disease (for example, diabetic nephropathy), hypertension induced nephropathy, glomerulosclerosis, proteinuria, nephrotic syndrome, polycystic kidney disease, autosomal dominant polycystic kidney disease, diabetic kidney disease, lupus nephritis; interstitial cystitis; periodontitis, gingivitis; pulmonary inflammation, sinusitis, pneumonia, bronchitis, asthma, bronchial asthma, allergic asthma, non-allergic asthma, allergic bronchopulmonary mycosis, aspirin-induced asthma, adult-onset asthma, asthma with fixed airflow obstruction, exercise-induced asthma, cough-variant asthma, work-related asthma, nighttime (nocturnal) asthma, asthma with obesity, eosinophilic asthma, steroid-resistant asthma/severe asthma, extrinsic asthma, intrinsic/cryptogenic asthma, Churg-Strauss syndrome, bronchiolitis, bronchiolitis obliterans, chronic obstructive pulmonary disease (COPD), interstitial lung disease (pulmonary fibrosis, idiopathic pulmonary fibrosis), acute lung injury, pulmonary fibrosis (for example, idiopathic pulmonary fibrosis or cystic fibrosis), chronic obstructive pulmonary disease, adult respiratory distress syndrome, acute lung injury, drug-induced lung injury; Meniere's disease; ocular disorders including, e.g., ocular inflammation, uveitis, dry eye/keratoconjunctivitis sicca, scleritis, episcleritis, keratitis/keratopathy, choroiditis, retinal vasculitis, optic neuritis, retinopathy (diabetic retinopathy, immune mediated retinopathy, macular degeneration, wet macular degeneration, dry (age related) macular degeneration); Mastocytosis, iron deficiency anemia, uremia, hypereosinophilic syndrome (HES), systemic mast cell disease (SMCD), myelodysplastic syndrome, idiopathic thrombocytic purpura; bone resorption diseases; Neurodegenerative disorders, neurological/neuromuscular disorders, e.g., multiple sclerosis, Parkinson's disease, Huntington's disease, amyotrophic lateral sclerosis (ALS) (familial ALS, sporadic ALS), Alzheimer's disease, myasthenia gravis, Lambert-Eaton myasthenic syndrome (LEMS), Guillain-Barret syndrome, meningitis, encephalitis, traumatic brain injury; nervous system damage, delusional parasitosis, dysregulation of neuronal processes and sensory perception, stroke/neuronal ischemia, spinal cord injury, peripheral neuropathy, tactile hallucinations, secondary damage after spinal cord injury, psychiatric disease; pain (acute pain, chronic pain, neuropathic pain, or fibromyalgia) paresthetica, nerve irritation, peripheral neuropathy; pruritus/itch (atopic pruritus, xerotic pruritus, pruritus associated with psoriasis/psoriatic itch/psoriasis-associated itch), acute pruritus, chronic pruritus, idiopathic pruritus, chronic idiopathic itch, biliary itch, hepatobiliary-associated itch, renal associated itch/renal itch, uremic itch, cholestasis, intrahepatic cholestasis of pregnancy, lichen simplex chronicus associated pruritus, lymphoma-associated itch, leukemia-associated itch, prurigo nodularis, atopic dermatitis-associated itch, atopic itch/atopic pruritus, bullous itch, brachioradial pruritus) neurogenic itch, neuropathic itch, notalgia paresthetica, pruritic popular eruption of HIV, psychogenic itch, swimmer's itch, pruritus or uremic itch, urticarial itch; dermatologic disorders, e.g., dermatologic drug reactions/drug eruptions, xerosis/dry skin, skin rash, skin sensitization, skin irritation, sunburn, shaving, body louse, head lice/pediculosis, pubic lice, cutaneous larva migrans, scabies, parasitic infection, insect infestation, urticaria/hives, papular uritcaria, insect bites, insect stings, dandruff, foreign objects or devices on skin, fungal infection, herpes, varicella/chicken pox, eosinophilic folliculitis, dermatosis of pregnancy/pruritic urticarial papules and plaques of pregnancy (PUPP), inflammatory dermatoses, neutrophilic dermatoses, histiocytoid neutrophilic dermatosis, bowel-bypass syndrome dermatosis, psoriasis/psoriasis vulgaris, lichen planus, lichen sclerosus, acne (acne vulgaris, comedonal acne, inflammatory acne, nodulo-cystic acne, scarring acne, acne keloidalis nuchae), atopies (allergic contact sensitization, allergic dermatitis) dermatitis (atopic dermatitis/eczema, contact dermatitis, photodermatitis, seborrheic dermatitis, stasis dermatitis, acute febrile neutrophilic dermatosis (Sweet's syndrome), chronic atypical neutrophilic dermatosis with lipodystrophy and elevated temperature syndrome (CANDLE Syndrome), hidradenitis suppurativa, hives, pyoderma gangrenosum, alopecia (eyebrow alopecia, intranasal hair alopecia, scarring alopecia (e.g., cicatricial alopecia, central centrifugal cicatricial alopecia, lichen planopilaris, frontal fibrosing alopecia, folliculitis decalvans), nonscarring alopecia (alopecia areata (AA) (patchy AA, alopecia totalis (AT), alopecia universalis (AU), ophiasis pattern alopecia areata, sisaihpo pattern alopecia areata)), androgenetic/androgenic alopecia (AGA)/male and female pattern AGA), telogen effluvium, tinea capitis, hypotrichosis (hereditary hypotrichosis simplex), lichen planopilaris (frontal fibrosing alopecia), punctate palmoplantar keratoderma, erythema elevatinum diutinum (EED), neutrophilic eccrine hidradenitis, palisading neutrophilic granulomatous dermatitis, neutrophilic urticarial dermatosis, vitiligo including segmental vitiligo (unisegmental vitiligo, bisegmental vitiligo, multisegmental vitiligo) non-segmental vitiligo (acral, facial, or acrofacial vitiligo, centrofacial vitiligo, mucosal vitiligo, confetti vitiligo, trichrome vitiligo, marginal inflammatory vitiligo, quadrichrome vitiligo, blue vitiligo, Koebner phenomenon, vulgaris vitiligo, generalized vitiligo, universal vitiligo), mixed vitiligo/nonsegmental associated with segmental vitiligo, focal vitiligo, solitary mucosal vitiligo or vitiligo with or without leukotricia (involvement of body hair); bullous diseases, immunobullous diseases (bullous pemphigoid, cicatricial pemphigoid, pemphigus vulgaris, linear IgA disease), gestational pemphigoid, xeroderma pigmentosum; disorders of fibrosis and scarring: fibroids, hepatic fibrosis, pulmonary fibrosis, idiopathic pulmonary fibrosis, low grade scarring such as, scleroderma, increased fibrosis, keloids, post-surgical scars; wound healing, surgical scarring, radiation induced fibrosis (for example, head and neck, gastrointestinal or pulmonary), CNS scarring, alimentary track or gastrointestinal fibrosis, renal fibrosis, hepatic or biliary fibrosis, liver fibrosis (for example, nonalcoholic steatohepatitis, hepatitis C, or hepatocellular carcinoma), cardiac fibrosis (for example, endomyocardial fibrosis or atrial fibrosis), ophthalmic scarring, fibrosclerosis, scar growth, wound or scab healing, keloid, mediastinal fibrosis, myelofibrosis, retroperitoneal fibrosis/Ormond's disease, progressive massive fibrosis, nephrogenic systemic fibrosis; Sjogren's syndrome, sarcoidosis, familial Mediterranean fever, Cryopyrin associated periodic syndrome (Muckle-Wells syndrome, familial cold auto-inflammatory syndrome/familial cold urticaria/TNF receptor associated periodic syndrome, neonatal-onset multisystem inflammatory disease), hyperoxia induced inflammations, reperfusion injury, post-surgical trauma, tissue injury, elevated temperature syndrome; diabetes (Type I diabetes, Type II diabetes)/diabetes mellitus, Hashimoto's thyroiditis, Graves' disease, Addison's disease, Castleman's disease, hyperparathyroidism, menopause, obesity, steroid-resistance, glucose intolerance, metabolic syndrome, thyroid illness, hypophysitis; systemic immune senescence; autoimmune atrophic gastritis, autoimmune atrophic gastritis of pernicious anemia, autoimmune encephalomyelitis, autoimmune orchitis, Goodpasture's disease, Sjogren's syndrome, autoimmune thrombocytopenia, sympathetic ophthalmia; secondary hematologic manifestations of autoimmune diseases (for example, anemias), autoimmune hemolytic syndromes (autoimmune hemolytic anemia), autoimmune and inflammatory hepatitis, autoimmune ovarian failure, autoimmune thrombocytopenia, silicone implant associated autoimmune disease, drug-induced autoimmunity, HIV-related autoimmune syndromes, metal-induced autoimmunity, autoimmune deafness, autoimmune thyroid disorders; allergy and allergic reactions including hypersensitivity reactions such as Type I hypersensitivity reactions, (e.g. including anaphylaxis), Type II hypersensitivity reactions (e.g. Goodpasture's Disease, autoimmune hemolytic anemia), Type III hypersensitivity reaction diseases (e.g. the Arthus reaction, serum sickness), and Type IV hypersensitivity reactions (e.g. contact dermatitis, allograft rejection); acute and chronic infection, sepsis syndromes (sepsis, septic shock, endotoxic shock, exotoxin-induced toxic shock, gram negative sepsis, gram positive sepsis, fungal sepsis, toxic shock syndrome); acute and chronic infection, sepsis syndromes (sepsis, septic shock, endotoxic shock, exotoxin-induced toxic shock, gram negative sepsis, gram positive sepsis, fungal sepsis, toxic shock syndrome); a rejection: graft vs. host reaction/graft vs. host disease, allograft rejections (for example, acute allograft rejection or chronic allograft rejection), early transplantation rejection; Malignancy, cancer, lymphoma, leukemia, multiple myeloma, a solid tumor, teratoma, metastatic and bone disorders, internal cancers, cancer of the: bone, mouth/pharynx, esophagus, larynx, stomach, intestine, colon, rectum, lung (for example, non-small cell lung cancer or small cell lung cancer), liver (hepatic), pancreas, nerve, brain (for example, glioma, glioblastoma multiforme, astrocytoma, neuroblastoma, and schwannomas), head and neck, throat, ovary, uterus, prostate, testis, bladder, kidney (renal), breast, gall bladder, cervix, thyroid, prostate, eye (ocular malignancies), and skin (melanoma, keratoacanthoma); as well as fibrotic cancers, fibroma, fibroadenomas, fibrosarcomas, a myeloproliferative disorder, neoplasm (hematopoietic neoplasm, a myeloid neoplasm, a lymphoid neoplasm (myelofibrosis, primary myelofibrosis, polycythemia vera, essential thrombocythemia)), leukemias (acute lymphocytic leukemia, acute and chronic myelogenous leukemia, chronic lymphocytic leukemia, acute lymphoblastic leukemia, chronic myelomonocytic leukemia (CMML), or promyelocytic leukemia), multiple myeloma and other myeloid malignancies (myeloid metaplasia with myelofibrosis (MMM), primary myelofibrosis (PMF), idiopathic myelofibrosis (IMF)), lymphomas (Hodgkin's disease, cutaneous lymphomas (cutaneous T-cell lymphoma, mycosis fungoides), lymphomas (for example, B-cell lymphoma, T-cell lymphoma, mantle cell lymphoma, hairy cell lymphoma, Burkitt's lymphoma, mast cell tumors, Hodgkin's disease or non-Hodgkin's disease); Kaposi's sarcoma, rhabdomyosarcoma, seminoma, teratocarcinoma, osteosarcoma, thyroid follicular cancer; increased accumulation of exogenous opioids or synthetic opioids, notalgia paraesthetica, obsessive-compulsive disorders, nostalgia associated with obsessive-compulsive disorders, and a combination thereof.

In some embodiments, additional exemplary disorders include, but are not limited to: complications from organ transplants (including xenotransplantation) such as graft vs. host reaction (for example, graft vs. host disease), allograft rejections (for example, acute allograft rejection or chronic allograft rejection), early transplantation, diabetes, a myeloproliferative disorder, a rejection (for example, acute allograft rejection); bone resorption diseases, asthma (e.g., bronchial asthma), atopy, autoimmune thyroid disorders, chronic atypical neutrophilic dermatosis with lipodystrophy and elevated temperature syndrome (CANDLE Syndrome), SAVI (stimulator of interferon genes (STING) associated vasculopathy with onset in infancy), ulcerative colitis, inflammatory bowel disease, Crohn's disease, celiac disease, ulcerative colitis, Behcet's disease, myasthenia gravis, nephropathies, and myocarditis, secondary hematologic manifestations of autoimmune diseases (for example, anemias), autoimmune hemolytic syndromes, autoimmune and inflammatory hepatitis, autoimmune ovarian failure, autoimmune orchitis, autoimmune thrombocytopenia, silicone implant associated autoimmune disease, drug-induced autoimmunity, HIV-related autoimmune syndromes; acute and chronic infection, sepsis syndromes, e.g., sepsis, septic shock, endotoxic shock, exotoxin-induced toxic shock, gram negative sepsis, gram positive sepsis, fungal sepsis, toxic shock syndrome; hyperoxia induced inflammations, reperfusion injury, post-surgical trauma, tissue injury, pain, e.g., acute pain, chronic pain, neuropathic pain, or fibromyalgia.

In some embodiments, said asthma is allergic asthma, non-allergic asthma, allergic bronchopulmonary mycosis, aspirin-induced asthma, adult-onset asthma, asthma with fixed airflow obstruction, exercise-induced asthma, cough-variant asthma, work-related asthma, nighttime (nocturnal) asthma, asthma with obesity, eosinophilic asthma, steroid-resistant asthma/severe asthma, extrinsic asthma, or intrinsic/cryptogenic asthma.

In some embodiments, said vitiligo is segmental vitiligo including unisegmental, bisegmental or multisegmental vitiligo, non-segmental vitiligo including acral, facial, or acrofacial vitiligo, centrofacial vitiligo, mucosal vitiligo, confetti vitiligo, trichrome vitiligo, marginal inflammatory vitiligo, quadrichrome vitiligo, blue vitiligo, Koebner phenomenon, vulgaris vitiligo, generalized vitiligo, universal vitiligo, mixed vitiligo (nonsegmental associated with segmental vitiligo), focal vitiligo, solitary mucosal vitiligo or vitiligo with or without leukotricia (involvement of body hair)

In some embodiments, the methods and compositions of the present invention encompass the prevention and treatment of the connective tissue and joint disorders selected from the group consisting of arthritis, rheumatoid arthritis, spondyloarthopathies, gouty arthritis, lumbar spondylarthrosis, carpal tunnel syndrome, canine hip dysplasia, systemic lupus erythematosus, juvenile arthritis, osteoarthritis, tendonitis and bursitis.

In some embodiments, the inflammatory disorder to be treated in accordance with the methods and compositions described herein is selected from rheumatoid arthritis, psoriatic arthritis, psoriasis, plaque psoriasis, gout, inflammatory bowel disease, hidradenitis suppurativa, Cryopyrin associated periodic syndrome (CAPS), pericarditis, including acute, chronic, and recurring pericarditis, ankylosing spondylitis, systemic juvenile idiopathic arthritis, systemic lupus erythematosus, multiple sclerosis, an inflammatory bone disorder, osteoarthritis, septic shock, endotoxic shock, endotoxin-induced toxic shock, toxic shock syndrome, sepsis, septic shock, atherosclerosis, diabetes, asthma, reperfusion injury, neuronal ischemia, stroke, graft versus host disease, allograft rejection, glomerulonephritis, pulmonary inflammation, chronic obstructive pulmonary disease (COPD), acute coronary syndrome, heart failure, atopic dermatitis, cancer (e.g., breast, pancreatic, colorectal, and lung cancer), fibrotic disease, cytokine release syndrome, and acute respiratory distress syndrome.

In some embodiments, the inflammatory condition that is treated in accordance with the methods described herein is arthritis, in particular rheumatoid arthritis. In some embodiments, the condition that is treated is hidradenitis suppurativa. In some embodiments, the inflammatory condition to be treated is gout. In some embodiments, the inflammatory condition to be treated is plaque psoriasis or psoriatic arthritis. In some embodiments, the inflammatory condition to be treated is ankylosing spondylitis. In some embodiments, the inflammatory condition to be treated is pericarditis, including acute pericarditis, recurrent pericarditis, and chronic pericarditis. In some embodiments, the inflammatory condition to be treated is Cryopyrin associated periodic syndrome (CAPS), including Muckle Wells Syndrome and Familial Cold Autoinflammatory Syndrome (FCAS). In some embodiments, the inflammatory condition to be treated is pyoderma gangrenosum. In some embodiments, the condition to be treated is inflammatory bowel disease, including Crohn's disease and ulcerative colitis. In some embodiments, the inflammatory condition to be treated is Stills disease, also referred to as juvenile idiopathic arthritis. In some embodiments, the inflammatory condition to be treated is atopic dermatitis. In some embodiments, the inflammatory condition to be treated is acute coronary syndrome. In some embodiments, the condition to be treated is heart failure. In some embodiments, the inflammatory condition to be treated is cancer, including, but not limited to, breast cancer, pancreatic cancer, colorectal cancer and lung cancer. In some embodiments, the inflammatory condition is cytokine release syndrome. In some embodiments, the inflammatory condition is acute respiratory distress syndrome.

In some embodiments, the present invention provides a method of treating inflammation in a patient in need thereof, the method comprising administered to the patient a provided compound or a pharmaceutically acceptable salt thereof. See, e.g., Mourey, Robert J., et al. “A benzothiophene inhibitor of mitogen-activated protein kinase-activated protein kinase 2 inhibits tumor necrosis factor α production and has oral anti-inflammatory efficacy in acute and chronic models of inflammation.” Journal of Pharmacology and Experimental Therapeutics 333.3 (2010). 797-807.

In some embodiments, the present invention provides a method of treating skin inflammation in a patient in need thereof, the method comprising administered to the patient a provided compound or a pharmaceutically acceptable salt thereof. See, e.g., Funding, Anne T. et al. “Reduced oxazolone-induced skin inflammation in MAPKAP kinase 2 knockout mice.” Journal of Investigative Dermatology 129.4 (2009): 891-898.

In some embodiments, the present invention provides a method of treating cardiac hypertrophy in a patient in need thereof, the method comprising administered to the patient a provided compound or a pharmaceutically acceptable salt thereof. See, e.g., Streicher, John M., et al. “MAPK-activated protein kinase-2 in cardiac hypertrophy and cyclooxygenase-2 regulation in heart.” Circulation research 106.8 (2010): 1434-1443.

In some embodiments, the present invention provides a method of treating inflammatory bowel disease in a patient in need thereof, the method comprising administered to the patient a provided compound or a pharmaceutically acceptable salt thereof. See, e.g., Zhang, Tao, et al. “MK2 is required for neutrophil-derived ROS production and inflammatory bowel disease.” Frontiers in medicine 7 (2020): 207; Strasser, Samantha Dale, et al. “Substrate-based kinase activity inference identifies MK2 as driver of colitis.” Integrative Biology 11.7 (2019): 301-314.

In some embodiments, the present invention provides a method of treating one or more of inflammation, hypertension, and oxidative stress in a patient in need thereof, the method comprising administered to the patient a provided compound or a pharmaceutically acceptable salt thereof. See, e.g., Ebrahimian, Talin, et al. “Mitogen-activated protein kinase-activated protein kinase 2 in angiotensin ii-induced inflammation and hypertension: regulation of oxidative stress.” Hypertension 57.2 (2011): 245-254.

In some embodiments, the present invention provides a method of treating osteoarthritis in a patient in need thereof, the method comprising administered to the patient a provided compound or a pharmaceutically acceptable salt thereof. See, e.g., Jones. S. W., et al. “Mitogen-activated protein kinase-activated protein kinase 2 (MK2) modulates key biological pathways associated with OA disease pathology.” Osteoarthritis and Cartilage 17.1 (2009): 124-131.

In some embodiments, the present invention provides a method of preventing or treating ischemic brain injury in a patient in need thereof, the method comprising administered to the patient a provided compound or a pharmaceutically acceptable salt thereof. See, e.g., Wang, Xinkang, et al. “Mitogen-activated protein kinase-activated protein (MAPKAP) kinase 2 deficiency protects brain from ischemic injury in mice.” Journal of Biological Chemistry 277.46 (2002): 43968-43972.

In some embodiments, the present invention provides a method of treating secondary damage after spinal cord injury in a patient in need thereof, the method comprising administered to the patient a provided compound or a pharmaceutically acceptable salt thereof. See, e.g., Ghasemlou, Nader, et al. “Mitogen-activated protein kinase-activated protein kinase 2 (MK2) contributes to secondary damage after spinal cord injury.” Journal of Neuroscience 30.41 (2010): 13750-13759.

In some embodiments, the present invention provides a method of treating Parkinson's disease in a patient in need thereof, the method comprising administered to the patient a provided compound or a pharmaceutically acceptable salt thereof. See, e.g., Thomas, Tobias, et al. “MAPKAP kinase 2-deficiency prevents neurons from cell death by reducing neuroinflammation-relevance in a mouse model of Parkinson's disease,” Journal of neurochemistry 105.5 (2008): 2039-2052.

In some embodiments, the present invention provides a method of treating atherosclerosis, hypercholesterolemia, or atherosclerosis and hypercholesterolemia in a patient in need thereof, the method comprising administered to the patient a provided compound or a pharmaceutically acceptable salt thereof. See, e.g., Jagavelu, Kumaravelu, et al. “Systemic deficiency of the MAP kinase-activated protein kinase 2 reduces atherosclerosis in hypercholesterolemic mice,” Circulation research 101.11 (2007): 1104-1112.

In some embodiments, the present invention provides a method of treating ventilator associated lung injury in a patient in need thereof, the method comprising administered to the patient a provided compound or a pharmaceutically acceptable salt thereof. See, e.g., Damarla, Mahendra, et al. “Mitogen activated protein kinase activated protein kinase 2 regulates actin polymerization and vascular leak in ventilator associated lung injury.” PloS one 4.2 (2009): e4600.

In some embodiments, the present invention provides a method of treating pancreatitis in a patient in need thereof, the method comprising administered to the patient a provided compound or a pharmaceutically acceptable salt thereof. See, e.g., Li, Yong-Yu, et al. “Regulation of HSP60 and the role of MK2 in a new model of severe experimental pancreatitis.” American Journal of Physiology—Gastrointestinal and Liver Physiology 297.5 (2009): G981-G989.

In some embodiments, the present invention provides a method of treating intestinal carcinogenesis in a patient in need thereof, the method comprising administered to the patient a provided compound or a pharmaceutically acceptable salt thereof. See, e.g., Henriques, Ana, Vasiliki Koliaraki, and George Kollias. “Mesenchymal MAPKAPK2/HSP27 drives intestinal carcinogenesis.” Proceedings of the National Academy of Sciences 115.24 (2018): E5546-E3555.

In some embodiments, the present invention provides a method of treating inflammation-induced colon cancer in a patient in need thereof, the method comprising administered to the patient a provided compound or a pharmaceutically acceptable salt thereof. See, e.g., Suarez-Lopez, Lucia, et al. “MAPKAP kinase-2 drives expression of angiogenic factors by tumor-associated macrophages in a model of inflammation-induced colon cancer.” Frontiers in Immunology (2021): 3929.

In some embodiments, the present invention provides a method of treating lung fibrosis in a patient in need thereof, the method comprising administered to the patient a provided compound or a pharmaceutically acceptable salt thereof. See, e.g., Liang, Jiurong, et al. “Mitogen-activated protein kinase-activated protein kinase 2 inhibition attenuates fibroblast invasion and severe lung fibrosis.” American journal of respiratory cell and molecular biology 60.1 (2019): 41-48.

In some embodiments, the present invention provides a method of lowering blood glucose, improving insulin sensitivity, or lowering blood glucose and improving insulin sensitivity in a patient in need thereof, the method comprising administered to the patient a provided compound or a pharmaceutically acceptable salt thereof. See, e.g., Ozcan, Lale, et al. “Treatment of obese insulin-resistant mice with an allosteric MAPKAPK2/3 inhibitor lowers blood glucose and improves insulin sensitivity.” Diabetes 64.10 (2015): 3396-3405.

In some embodiments, the present invention provides a method of treating melanoma in a patient in need thereof, the method comprising administering to the patient a provided compound or a therapeutically acceptable salt thereof. See, e.g., Soukup, Klara, et al. “Loss of MAPK-activated protein kinase 2 enables potent dendritic cell-driven anti-tumour T cell response.” Scientific reports 7.1 (2017): 1-15.

In some embodiments, the methods described herein are used to treat patients with disorders arising from dysregulated cytokine, enzymes and/or inflammatory mediator production, stability, secretion, posttranslational processing. In some embodiments, the methods described herein are used to treat patients having cytokine release syndrome, which is a systemic inflammatory response triggered by a variety of factors including infections (e.g., viral infection) and certain drugs (CAR T-cell therapy). Examples of cytokines that may be dysregulated include interleukins 1, 2, 6, 8, 10, 12, 17, 22 and 23 along with tumor necrosis factor alpha and interferons alpha, beta and gamma. Examples of inflammatory mediators that may be dysregulated include nitric oxide, prostaglandins and leukotrienes. Examples of enzymes include cyclo-oxygenase, nitric oxide synthase and matrix metalloprotease.

In some embodiments, the methods and compositions of the present invention encompass the prevention and treatment of the neoplasia disorders selected from the group consisting of acral lentiginous melanoma, actinic keratoses, adenocarcinoma, adenoid cycstic carcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cholangiocarcinoma, chondosarcoma, choriod plexus papilloma/carcinoma, clear cell carcinoma, skin cancer, brain cancer, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, fibrolamellar, focal nodular hyperplasia, gallbladder cancer, gastrinoma, germ cell tumors, gestational trophoblastic tumor, glioblastoma, glioma, glucagonoma, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, insulinoma, intaepithelial neoplasia, interepithelial squamous cell neoplasia, intraocular melanoma, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neuroepithelial adenocarcinoma nodular melanoma, non-Hodgkin's lymphoma, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous carcinoma, squamous cell carcinoma, submesothelial, superficial spreading melanoma, supratentorial primitive neuroectodermal tumors, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor.

In some embodiments, the methods and compositions of the present invention encompass the prevention and treatment of the cardiovascular disorders selected from the group consisting of myocardial ischemia, hypertension, hypotension, heart arrhythmias, pulmonary hypertension, hypokalemia, cardiac ischemia, myocardial infarction, cardiac remodeling, cardiac fibrosis, myocardial necrosis, aneurysm, arterial fibrosis, embolism, vascular plaque inflammation, vascular plaque rupture, bacterial-induced inflammation and viral induced inflammation, edema, swelling, fluid accumulation, cirrhosis of the liver, Bartter's syndrome, myocarditis, arteriosclerosis, atherosclerosis, calcification (such as vascular calcification and valvar calcification), coronary artery disease, heart failure, congestive heart failure, shock, arrhythmia, left ventricular hypertrophy, angina, diabetic nephropathy, kidney failure, eye damage, vascular diseases, migraine headaches, aplastic anemia, cardiac damage, diabetic cardiac myopathy, renal insufficiency, renal injury, renal arteriopathy, peripheral vascular disease, left ventricular hypertrophy, cognitive dysfunction, stroke, and headache.

In some embodiments, the methods and compositions of the present invention encompass the prevention and treatment of the metabolic disorders selected from the group consisting of obesity, overweight, type I and type II diabetes, hypothyroidism, and hyperthyroidism.

In some embodiments, the methods and compositions of the present invention encompass the prevention and treatment of the respiratory disorders selected from the group consisting of asthma, bronchitis, chronic obstructive pulmonary disease (COPD), cystic fibrosis, pulmonary edema, pulmonary embolism, pneumonia, pulmonary sarcoisosis, silicosis, pulmonary fibrosis, respiratory failure, acute respiratory distress syndrome and emphysema.

In some embodiments, the methods and compositions of the present invention encompass the prevention and treatment of the angiogenesis-related disorders selected from the group consisting of angiofibroma, neovascular glaucoma, arteriovenous malformations, arthritis, osler-weber syndrome, atherosclerotic plaques, psoriasis, corneal graft neovascularization, pyogenic granuloma, delayed wound healing, retrolental fibroplasias, diabetic retinopathy, scleroderma, granulations, solid tumors, hemangioma, trachoma, hemophilic joints, vascular adhesions, hypertrophic scars, age-related macular degeneration, coronary artery disease, stroke, cancer, AIDS complications, ulcers and infertility.

In some embodiments, the methods and compositions of the present invention encompass the prevention and treatment of the infectious diseases and disorders selected from the group consisting of viral infections, bacterial infections, prion infections, spirochetes infections, mycobacterial infections, rickettsial infections, chlamydial infections, parasitic infections and fungal infections.

In some embodiments, the methods and compositions of the present invention encompass the prevention and treatment of the infectious diseases and disorders selected from the group consisting of hepatitis, HIV (AIDS), small pox, chicken pox, common cold, bacterial influenza, viral influenza, warts, oral herpes, genital herpes, herpes simplex infections, herpes zoster, bovine spongiform encephalopathy, septicemia, streptococcus infections, staphylococcus infections, anthrax, severe acquired respiratory syndrome (SARS), malaria, African sleeping sickness, yellow fever, chlamydia, botulism, canine heartworm, rocky mountain spotted fever, lyme disease, cholera, syphilis, gonorrhea, encephalitis, pneumonia, conjunctivitis, yeast infections, rabies, dengue fever, Ebola, measles, mumps, rubella, West Nile virus, meningitis, gastroenteritis, tuberculosis, hepatitis, and scarlet fever.

In some embodiments, the methods and compositions of the present invention encompass the prevention and treatment of the neurological and neurodegenerative disorders selected from the group consisting of headaches, migraine headaches, Alzheimer's disease, Parkinson's disease, dementia, memory loss, senility, amyotrophy, ALS, amnesia, seizures, multiple sclerosis, muscular dystrophies, epilepsy, schizophrenia, depression, anxiety, attention deficit disorder, hyperactivity, bulimia, anorexia nervosa, anxiety, autism, phobias, spongiform encephalopathies, Creutzfeldt-Jakob disease, Huntington's Chorea, ischemia, obsessive-compulsive disorder, manic depression, bipolar disorders, drug addiction, alcoholism and smoking addiction.

In some embodiments, the methods and compositions of the present invention encompass the prevention and treatment of the dermatological disorders selected from the group consisting of acne, psoriasis, eczema, burns, poison ivy, poison oak and dermatitis.

In some embodiments, the methods and compositions of the present invention encompass the prevention and treatment of the surgical disorders selected from the group consisting of pain and swelling following surgery, infection following surgery and inflammation following surgery.

In some embodiments, the methods and compositions of the present invention encompass the prevention and treatment of the gastrointestinal disorders selected from the group consisting of inflammatory bowel disease, irritable bowel syndrome, Crohn's disease, gastritis, irritable bowel syndrome, diarrhea, constipation, dysentery, ulcerative colitis, gastric esophageal reflux, gastric ulcers, gastric varices, ulcers, and heartburn.

In some embodiments, the methods and compositions of the present invention encompass the prevention and treatment of the otic disorders selected from the group consisting of otic pain, inflammation, otorrhea, otalgia, fever, otic bleeding, Lermoyez's syndrome, Meniere's disease, vestibular neuronitis, benign paroxysmal positional vertigo, herpes zoster oticus, Ramsay Hunt's syndrome, viral neuronitis, ganglionitis, geniculate herpes, labyrinthitis, purulent labyrinthitis, viral endolymphatic labyrinthitis, perilymph fistulas, noise-induced hearing loss, presbycusis, drug-induced ototoxicity, acoustic neuromas, aerotitis media, infectious myringitis, bullous myringitis, otitis media, otitis media with effusion, acute otitis media, secretory otitis media, serous otitis media, acute mastoiditis, chronic otitis media, otitis extema, otosclerosis, squamous cell carcinoma, basal cell carcinoma, nonchromaffin paragangliomas, chemodectomas, globus jugulare tumors, globus tympanicum tumors, external otitis, perichondritis, aural eczematoid dermatitis, malignant external otitis, subperichondrial hematoma, ceruminomas, impacted cerumen, sebaceous cysts, osteomas, keloids, otalgia, tinnitus, vertigo, tympanic membrane infection, typanitis, otic furuncles, otorrhea, acute mastoiditis, petrositis, conductive and sensorineural hearing loss, epidural abscess, lateral sinus thrombosis, subdural empyema, otitic hydrocephalus, Dandy's syndrome, bullous myringitis, cerumen-impacted, diffuse external otitis, foreign bodies, keratosis obturans, otic neoplasm, otomycosis, trauma, acute barotitis media, acute eustachian tube obstruction, post-otic surgery, postsurgical otalgia, cholesteatoma, conductive and sensorineural hearing loss, epidural abscess, lateral sinus thrombosis, subdural empyema and otitic hydrocephalus.

In some embodiments, the methods and compositions of the present invention encompass the prevention and treatment of the ophthalmic disorders selected from the group consisting of retinopathies, uveitis, ocular photophobia, acute injury to the eye tissue, conjunctivitis, age-related macular degeneration diabetic retinopathy, detached retina, glaucoma, vitelliform macular dystrophy type 2, gyrate atrophy of the choroid and retina, conjunctivitis, corneal infection, fuchs' dystrophy, iridocomeal endothelial syndrome, keratoconus, lattice dystrophy, map-dot-fingerprint dystrophy, ocular herpes, pterygium, myopia, hyperopia, and cataracts.

In some embodiments, the methods and compositions of the present invention encompass the prevention and treatment of menstrual cramps, kidney stones, minor injuries, wound healing, vaginitis, candidiasis, sinus headaches, tension headaches, dental pain, periarteritis nodosa, thyroiditis, myasthenia gravis, multiple sclerosis, sarcoidosis, nephrotic syndrome, Bahcet's syndrome, polymyositis, gingivitis, hypersensitivity, swelling occurring after injury, closed head injury, liver disease, and endometriosis.

In some embodiments, the present invention provides a method of treating a disease, disorder, or condition that is mediated by pro-inflammatory molecules such as TNFα, IL-1, and IL-6 in a patient in need thereof, the method comprising administering to the patient a compound of the present invention or a therapeutically acceptable salt thereof.

In some embodiments, the present invention provides a method of treating pain in a patient in need thereof, the method comprising administering to the patient a compound of the present invention or a therapeutically acceptable salt thereof.

In some embodiments, the present invention provides a method of treating inflammation in a patient in need thereof, the method comprising administering to the patient a compound of the present invention or a therapeutically acceptable salt thereof.

In some embodiments, the present invention provides a method of treating tissue damage in a patient in need thereof, the method comprising administering to the patient a compound of the present invention or a therapeutically acceptable salt thereof.

In some embodiments, the present invention provides a method of treating arthritis in a patient in need thereof, the method comprising administering to the patient a compound of the present invention or a therapeutically acceptable salt thereof.

It is believed that a provided compound or a pharmaceutically acceptable salt thereof may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

Co-Administration with One or More Other Therapeutic Agent(s)

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif©), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and MK2ins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxoMK2 (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxoMK2 (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In some embodiments, the present invention provides a method of treating HIV comprising administering to a patient in need thereof a provided compound and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenstrom's macroglobulinemia comprising administering to a patient in need thereof a provided compound and one or more additional therapeutic agents selected from chlorambucil (Leukeran®), cyclophosphamide (Cytoxan®, Neosar®), fludarabine (Fludara®), cladribine (LeuMK2 in®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinoMK2 (Zolinza®, Merck); romidepsin (Istodax®, Celgene); panobinoMK2 (Farydak®, Novartis); belinoMK2 (Beleodaq®, Spectrum Pharmaceuticals); entinoMK2 (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFß). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, proMK2e and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFß trap compound (NCT02699515); and (NCT02517398). M7824 is comprised of a fully human IgG1 antibody against PD-L1 fused to the extracellular domain of human TGF-beta receptor II, which functions as a TGFß “trap.”

In some embodiments, one or more other therapeutic agent is selected from glembatumumab vedotin-monomethyl auriMK2 in E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

In some embodiments, the present invention provides a method of treating Alzheimer's disease comprising administering to a patient in need thereof a provided compound and one or more additional therapeutic agents selected from donepezil (Aricept®), rivastigmine (Excelon©), galantamine (Razadyne®), tacrine (Cognex©), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, proMK2itis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, proMK2e cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic MK2es, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, proMK2e, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, proMK2e hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infeMK2ion (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

The compounds and compositions, according to the method of the present invention, may be administered using any amount and any route of administration effective for treating or lessening the severity of a cancer, an autoimmune disorder, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting protein kinase activity or degrading a protein kinase in a biological sample comprising the step of contacting said biological sample with a compound of this invention, or a composition comprising said compound.

According to another embodiment, the invention relates to a method of inhibiting or degrading MK2, or a mutant thereof, activity in a biological sample comprising the step of contacting said biological sample with a compound of this invention, or a composition comprising said compound.

The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

Inhibition and/or degradation of a MK2 protein, or a protein selected from MK2, or a mutant thereof, activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of degrading a protein kinase and/or inhibiting protein kinase activity in a patient comprising the step of administering to said patient a compound of the present invention, or a composition comprising said compound.

According to another embodiment, the invention relates to a method of degrading and/or inhibiting MK2, or a mutant thereof, activity in a patient comprising the step of administering to said patient a compound of the present invention, or a composition comprising said compound. In other embodiments, the present invention provides a method for treating a disorder mediated by MK2, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™ Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™ Teniposide is marketed under the trade name VM 26-Bristol Doxorubicin is marketed under the trade name Acriblastin™ or Adriamycin™. Epirubicin is marketed under the trade name Farmorubicin™ Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof, see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, BryoMK2 in 1, Perifosine; llmofosine; RO 318220 and RO 320432; GO 6976; lsis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); l) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-Cβ, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2- arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™ Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomeMK2 in.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™); carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimaMK2 and its orally bioavailable analogue marimaMK2 (BB-2516), prinomaMK2 (AG3340), metaMK2 (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan©), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), TrichoMK2 in A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2- propenamide, or a pharmaceutically acceptable salt thereof, especially the lactate salt. SomatoMK2 in receptor antagonists as used herein refer to compounds which target, treat or inhibit the somatoMK2 in receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentoMK2 in. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; AngioMK2 in™; EndoMK2 in™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

AngioMK2ic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's MK2us after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-0, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13/69264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgGI, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadoMK2 (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadoMK2, epratuzumab, indoximod, inotuzumab ozogamicin, intelumumab, ipilimumab, isatuximab, lambrolizumab, MEDI4736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metaMK2ic castrate-resistant (hormone-refractory) proMK2e cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); proMK2e cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metaMK2ic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the contents of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a MK2istically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺(αβ) T cells), CD160 (also referred to as BY55), CGEN-15049, CHK 1 and CHK2 kinases, A2aR, and various B-7 family ligands. B7 family ligands include, but are not limited to, B7- 1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PDl antibody), CT-011 (anti-PDl antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgGI anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metaMK2ic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, proMK2e cancer, endometrial cancer, metaMK2ic cancer in the liver, liver cancer, large B-cell lymphoma, ovarian cancer, cervical cancer, metaMK2ic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metaMK2ic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metaMK2ic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metaMK2ic proMK2e cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgGI Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

In some embodiments, MK23 inhibition/degradation can significantly enhance CDN-induced STING signaling and antitumor immunity (Pei et al., Can. Lett. 2019, 450:110).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metaMK2ic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

Analytical instruments Table:

| | |
|---|---|
| LCMS | Shimadzu UFLC MS: LCMS-2020 |
| | Agilent Technologies 1200 series MS: Agilent Technologies 6110 |
| | Agilent Technologies 1200 series MS: LC/MSD VL |
| NMR | br UKER AVANCE III/400; Frequency (MHz) 400.13; Nucleus: 1H; |
| | Number of Transients: 8 |
| Prep-HPLC | Gilson GX-281 systems: instruments GX-A, GX-B, GX-C, GX-D, GX-E, |
| | GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH⁺] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH⁺] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH₃.H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19) mm, 5μ. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

### INTERMEDIATES

### 2′-(2-chloropyrimidin-4-yl)-5′,6′-dihydrospiro[cyclopropane-1,7′-pyrrolo[3,2-c]pyridin]-4′(1′H)-one (Intermediate A)

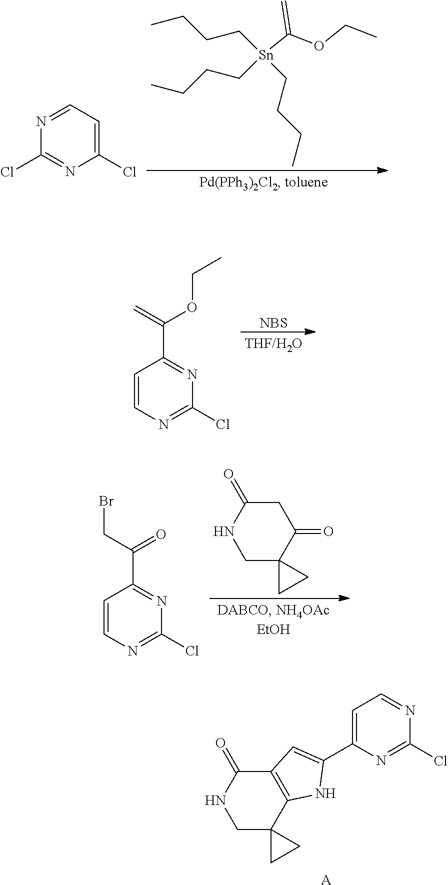

Step 1—2-Chloro-4-(1-ethoxyvinyl)pyrimidine. To a solution of 2,4-dichloropyrimidine (10 g, 67.1 mmol) in toluene (100 mL) was added tributyl(1-ethoxyvinyl)stannane (26.7 g, 73.8 mmol, 24.9 mL, CAS #97674-02-7) and Pd(PPh₃)₂Cl₂ (3.77 g, 5.37 mmol) at 25° C. under N₂ atmosphere. The reaction was stirred at 80° C. for 3 hrs. On completion, the reaction mixture was quenched with KF (10 g), and the mixture was stirred at 25° C. for 1 hr. Then the mixture was filtered and concentrated under reduced pressure to give a residue. The residue was purified by column chromatography (SiO₂, Petroleum ether/Ethyl acetate=20/1 to 10/1) to give the title compound (11 g, 89% yield) as a yellow solid. LC-MS (ESI⁺) m/z 185.1. (M+H).

Step 2—2-Bromo-1-(2-chloropyrimidin-4-yl)ethanone. To a solution of 2-chloro-4-(1-ethoxyvinyl)pyrimidine (5.00 g, 27.1 mmol) in THF (40 mL) and H₂O (16 mL) was added NBS (5.30 g, 29.8 mmol) at 25° C., then the mixture was stirred at 25° C. for 4 hrs. On completion, the reaction mixture was quenched with water (50 mL) and extracted by ethyl acetate (30×3 mL). The extracts were washed by brine (50 mL) and dried over anhydrous sodium sulfate, filtered and concentrated in vacuo to give the title compound (7 g) as a yellow solid. LC-MS (ESI⁺) m/z 236.9. (M+H)⁺.

Step 3—2′-(2-Chloropyrimidin-4-yl)-5′,6′-dihydrospiro[cyclopropane-1,7′-pyrrolo[3,2-c]pyridin]-4′(1′H)-one. To a solution of 5-azaspiro[2.5]octane-6,8-dione (4.43 g, 31.9 mmol) and 2-bromo-1-(2-chloropyrimidin-4-yl)ethanone (5 g, 21.2 mmol) in EtOH (80 mL) was added NH₄OAc (16.4 g, 212 mmol) at 25° C., then the mixture was stirred at 25° C. for 1 hr. On completion, the reaction mixture was filtered and concentrated in vacuo to give the crude residue. The residue was purified by column chromatography (SiO₂, DCM:MeOH=20/1 to 10/1) to give the title compound (3 g, 41% yield) as a yellow solid. LC-MS (ESI⁺) m/z 275.1. (M+H)⁺.

### 2′-(2-(2-fluoro-4-(piperazin-1-yl)phenyl)pyrimidin-4-yl)-5′,6′-dihydrospiro[cyclopropane-1,7′-pyrrolo[3,2-c]pyridin]-4′(1′H)-one (Intermediate B)

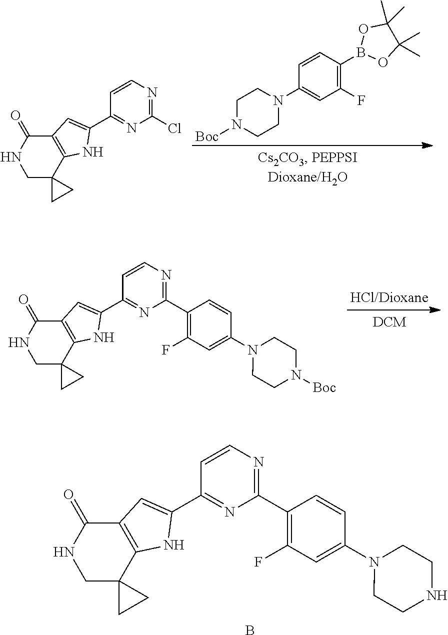

Step 1—Tert-butyl 4-(3-fluoro-4-(4-(4′-oxo-1′,4′,5′,6′-tetrahydrospiro[cyclopropane-1,7′-pyrrolo[3,2-c]pyridin]-2′-yl)pyrimidin-2-yl)phenyl)piperazine-1-carboxylate. A mixture of 2-(2-chloropyrimidin-4-yl)spiro[5,6-dihydro-1H-pyrrolo[3,2-c]pyridine-7,1′-cyclopropane]-4-one (300 mg, 1.09 mmol, Intermediate A), tert-butyl 4-[3-fluoro-4-(4,4,5,5-tetramethyl-1,3,2-dioxaborolan-2-yl)phenyl]piperazine-1-carboxylate (532 mg, 1.31 mmol, CAS #1146950-53-9), Cs₂CO₃ (711 mg, 2.18 mmol), and 1,3-bis[2,6-bis(1-propylbutyl)phenyl]-4,5-dichloro-2H-imidazol-1-ium-2-ide;3-chloropyridine; dichloropalladium (106 mg, 109 umol, CAS #1814936-54-3) in dioxane (2 mL) and H₂O (0.5 mL) was degassed and purged with N₂ three times. Then the mixture was stirred at 80° C. for 2 hrs under N₂ atmosphere. On completion, the reaction mixture was concentrated under reduced pressure to give a residue which was purified by prep-HPLC (FA condition) to give the title compound (600 mg, 78% yield, FA) as a yellow solid. LC-MS (ESI⁺) m/z 519.1 (M+H)⁺.

Step 2—2′-(2-(2-fluoro-4-(piperazin-1-yl)phenyl)pyrimidin-4-yl)-5′,6′-dihydrospiro[cyclopropane-1,7′-pyrrolo[3,2-c]pyridin]-4′(1′H)-one. To a solution of tert-butyl 4-[3-fluoro-4-[4-(4-oxospiro[5,6-dihydro-1H-pyrrolo[3,2-c]pyridine-7,1′-cyclopropane]-2-yl)pyrimidin-2-yl]phenyl]piperazine-1-carboxylate (600 mg, 1.06 mmol, FA) in DCM (5 mL) was added HCl/Dioxane (4 M, 1.52 mL). The mixture was stirred at 25° C. for 1 hr. On completion, the reaction mixture was concentrated under reduced pressure to give the title compound (500 mg, HCl) as a yellow solid. LC-MS (ESI⁺) m/z 419.1 (M+H)⁺.

### [1-[(4-Methoxyphenyl) methyl]-2,6-dioxo-3-piperidyl] trifluoromethanesulfonate (Intermediate C)

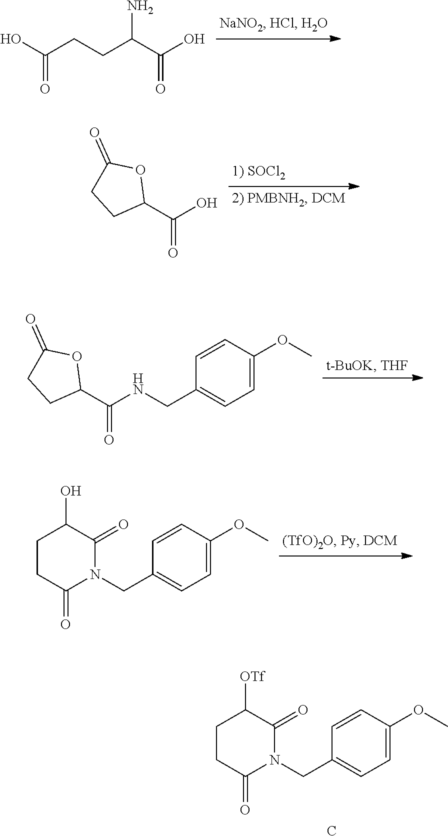

Step 1—5-Oxotetrahydrofuran-2-carboxylic acid. To a solution of 2-aminopentanedioic acid (210 g, 1.43 mol, CAS #617-65-2) in H₂O (800 mL) and HCl (12 M, 210 mL) was added a solution of NaNO₂ (147 g, 2.13 mol) in H₂O (400 mL) at −5° C. The mixture was stirred at 15° C. for 12 hrs. On completion, the mixture was concentrated and then dissolved in EA (500 mL) and filtered and washed with EA (3×100 mL). The filtrate and washed solution were dried over Na₂SO₄, filtered and concentrated in vacuo to give the title compound (200 g, crude) as yellow oil. ¹H NMR (400 MHz, CDCl₃) δ 6.43 (s, 1H), 5.02-4.95 (m, 1H), 2.67-2.38 (m, 4H)

Step 2—N-[(4-methoxyphenyl)methyl]-5-oxo-tetrahydrofuran-2-carboxamide. To 5-oxotetrahydrofuran-2-carboxylic acid (120 g, 922 mmol) was added SOCl₂ (246 g, 2.07 mol) at 0° C. slowly. The mixture was stirred at 85° C. for 3 hrs, and then the mixture was stirred at 15° C. for 6 hrs. The mixture was concentrated in vacuo. The residue was dissolved in dry DCM (1 L) at 0° C. under N₂. After that a solution of Et₃N (187 g, 1.84 mol) and 4-methoxybenzylamine (101 g, 738 mmol) in DCM (400 mL) was added, then the mixture was stirred at 15° C. for 3 hrs. On completion, water (600 mL) was added and the mixture was extracted with DCM (3×300 mL). The combined organic phase was washed with 0.5 M HCl (500 mL), brine (500 mL), dried over with anhydrous sodium sulfate and filtered. The filtrate was concentrated in vacuo and the residue was purified by flash silica gel chromatography (PE:EA=1:1) to give the title compound (138 g, 60% yield) as a yellow solid. ¹H NMR (400 MHz, CDCl₃) δ 7.22-7.20 (d, J=8.0, 1H), 6.89-6.87 (d, J=8.0, 1H), 4.90-4.86 (m, 1H), 4.47-4.4.36 (m, 2H) 3.81 (s, 3H), 2.67-2.64 (m, 1H), 2.59-2.54 (m, 2H), 2.40-2.38 (m, 1H); LC-MS (ESI⁺) m/z 272.0 (M+Na)⁺.

Step 3—3-Hydroxy-1-[(4-methoxyphenyl)methyl]piperidine-2,6-dione. A solution of N-[(4-methoxyphenyl)methyl]-5-oxo-tetrahydrofuran-2-carboxamide (138 g, 553 mmol) in anhydrous THF (1500 mL) was cooled to −78° C. Then, t-BuOK (62.7 g, 559 mmol) in a solution of anhydrous THF (1000 mL) was added dropwise slowly at −78° C. under nitrogen atmosphere. The resulting reaction mixture was stirred at −40° C. for 1 hr. On completion, the reaction mixture was quenched with saturated NH₄Cl solution (100 mL). The mixture was extracted with ethyl acetate (3×1500 mL). The combined organic layer was washed with brine (300 mL), dried over anhydrous sodium sulfate, filtered and the filtrate was concentrated in vacuo. The residue was purified by silica gel chromatography (PE:EA=1:1) to give the title compound (128 g, 92% yield) as a white solid. ¹H NMR (400 MHz, CDCl₃) δ 7.39-7.32 (m, 2H), 6.89-6.81 (m, 2H), 4.91 (s, 2H), 4.17-4.11 (m, 1H), 3.80 (s, 3H), 3.54 (s, 1H), 2.98-2.87 (m, 1H), 2.73-2.60 (m, 1H), 2.26-2.20 (m, 1H), 1.80 (dq, J=4.8, 13.1 Hz, 1H).

Step 4—[1-[(4-Methoxyphenyl) methyl]-2,6-dioxo-3-piperidyl] trifluoromethanesulfonate. To a solution of 3-hydroxy-1-[(4-methoxyphenyl) methyl] piperidine-2, 6-dione (43.0 g, 173 mmol) and pyridine (27.3 g, 345 mmol) in DCM (500 mL) was added trifluoromethylsulfonyl trifluoromethanesulfonate (73.0 g, 258 mmol) dropwise at 0° C. The mixture was stirred at −10° C. for 1.5 hours under N₂. On completion, the mixture was concentrated in vacuo. The residue was purified by column chromatography on silica gel (PE:EA=20:1/8:1) to give the title compound (45.0 g, 68% yield) as a light yellow gum. ¹H NMR (400 MHz, CDCl₃) δ 7.36 (d, J=8.4 Hz, 2H), 6.85-6.82 (m, 2H), 5.32-5.28 (m, 1H), 4.91 (s, 2H), 3.79 (s, 3H), 3.02-2.97 (m, 1H), 2.79-2.74 (m, 1H), 2.41-2.35 (m, 2H).

### 5-Bromo-3-methyl-1H-benzimidazol-2-one (Intermediate D)

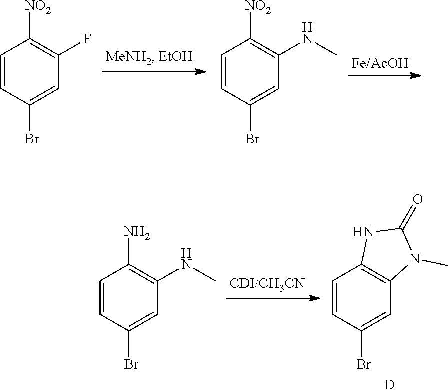

Step 1—5-Bromo-N-methyl-2-nitro-aniline. 4-bromo-2-fluoro-1-nitro-benzene (230 g, 1.05 mol, CAS #321-23-3) was added to a solution of methylamine in tetrahydrofuran (2 M, 1.51 L). The mixture was stirred at 15° C. for 10 minutes. On completion, the mixture was diluted with H₂O (250 mL) and extracted with EtOAc (3×300 mL). The combined organic layers were washed with brine (300 mL), dried over Na₂SO₄, filtered and concentrated in vacuo to give the title compound (200 g, 83% yield) as a yellow solid. ¹H NMR (400 MHz, DMSO-d₆) δ 8.22 (s, 1H), 7.98 (d, J=9.2 Hz, 1H), 7.16 (d, J=1.6 Hz, 1H), 6.82 (dd, J=8.4, 1.6 Hz, 1H), 2.95 (d, J=4.8 Hz, 3H).

Step 2—4-Bromo-N2-methyl-benzene-1,2-diamine. To a mixture of 5-bromo-N-methyl-2-nitro-aniline (200 g, 865 mmol) in EtOAc (1 L) and H₂O (500 mL) was added AcOH (1.00 L). The mixture was warmed to 50° C., and then Fe (174 g, 3.11 mol) was added to the reaction mixture. After that, the reaction mixture was stirred at 80° C. for 6 hours. On completion, the mixture was filtered through celite. The filtrate was concentrated in vacuo and the residue was diluted with H₂O (250 mL) and extracted with EtOAc (3×300 mL). The combined organic layers were washed with aq. NaHCO₃ and brine (300 mL), dried over Na₂SO₄, filtered and concentrated in vacuo. The residue was purified by flash silica gel chromatography to give the title compound (130 g, 75% yield) as a black oil. ¹H NMR (400 MHz, DMSO-d₆) δ 6.55-6.52 (m, 1H), 6.48-6.45 (m, 1H), 6.43-6.42 (m, 1H), 4.89-4.88 (m, 1H), 4.61 (s, 2H), 2.70 (d, J=4.0 Hz, 3H).

Step 3—5-Bromo-3-methyl-1H-benzimidazol-2-one. To a solution of 4-bromo-N2-methyl-benzene-1,2-diamine (110 g, 547 mmol) in CH₃CN (1.3 L) was added CDI (177 g, 1.09 mol). The mixture was stirred at 80° C. for 6 hours under N₂. On completion, the mixture was concentrated in vacuo. The mixture was diluted with H₂O (1.0 L) and filtered. The filter cake was washed with water (3×200 mL) and dried in vacuo to give the title compound (106 g, 85% yield) as a white solid. ¹H NMR (400 MHz, DMSO-d₆) δ 11.00 (s, 1H), 7.33 (s, 1H), 7.13 (d, J=8.0 Hz, 1H), 6.92 (d, J=8.0 Hz, 1H), 3.27 (s, 3H).

### 3-(5-bromo-3-methyl-2-oxo-benzimidazol-1-yl)piperidine-2,6-dione (Intermediate E)

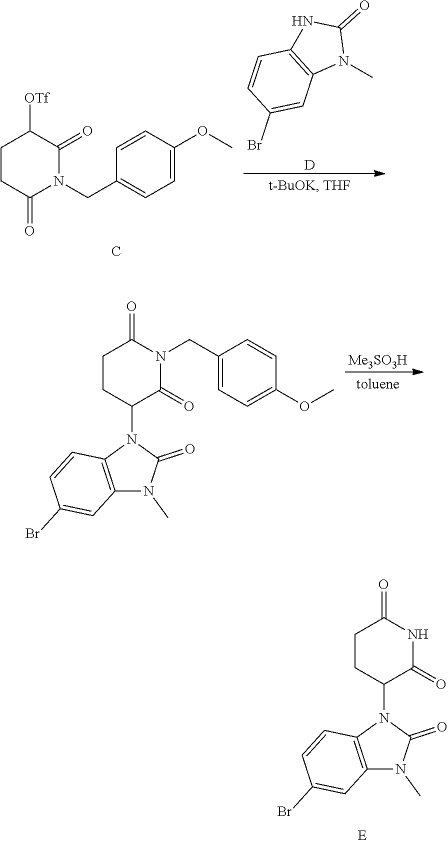

Step 1—3-(5-Bromo-3-methyl-2-oxo-benzimidazol-1-yl)-1-[(4-methoxyphenyl)methyl]piperidine-2,6-dione. To a solution of 5-bromo-3-methyl-1H-benzimidazol-2-one (4.90 g, 21.6 mmol, Intermediate D) in THF (300 mL) was added t-BuOK (3.63 g, 32.3 mmol) at 0° C. The mixture was stirred at 0-10° C. for 1 hour under N₂. Then a solution of [1-[(4-methoxyphenyl) methyl]-2, 6-dioxo-3-piperidyl] trifluoromethanesulfonate (9.87 g, 25.9 mmol, Intermediate C) in THF (100 mL) was added to the reaction mixture at 0-10° C. during 30 minutes. The mixture was stirred at 0-10° C. for 30 minutes under N₂. An additional solution of [1-[(4-methoxyphenyl) methyl]-2, 6-dioxo-3-piperidyl] trifluoromethanesulfonate (2.47 g, 6.47 mmol) in THF (20 mL) was added to the reaction mixture at 0-10° C. dropwise. The mixture was then stirred at 0-10° C. for another 30 minutes under N₂. On completion, the reaction was quenched water (400 mL) and extracted with EA (3×200 mL). The combined organic layer was concentrated in vacuo. The residue was triturated with EA (80 mL) and filtered. The filter cake was collected and dried in vacuo to give the title compound (6.70 g, 67% yield) as light yellow solid. The filtrate was also concentrated in vacuo and the residue was purified by column chromatography to give another batch of the title compound (1.80 g, 18% yield) as light yellow solid. ¹H NMR (400 MHz, DMSO-d₆) δ 7.47 (d, J=1.6 Hz, 1H), 7.21-7.16 (m, 3H), 7.01 (d, J=8.0 Hz, 1H), 6.85 (d, J=8.8 Hz, 2H), 5.55-5.51 (m, 1H), 4.84-4.73 (m, 2H), 3.72 (s, 3H), 3.33 (s, 3H), 3.04-3.00 (m, 1H), 2.83-2.67 (m, 2H), 2.07-2.05 (m, 1H).

Step 2—3-(5-Bromo-3-methyl-2-oxo-benzimidazol-1-yl)piperidine-2,6-dione. To a mixture of 3-(5-bromo-3-methyl-2-oxo-benzimidazol-1-yl)-1-[(4-methoxyphenyl)methyl] piperidine-2,6-dione (8.50 g, 18.6 mmol) in toluene (50 mL) was added methanesulfonic acid (33.8 g, 351 mmol, 25 mL) at room temperature (15° C.). The mixture was stirred at 120° C. for 2 hours. On completion, the reaction mixture was cooled to room temperature and concentrated in vacuo. The residue was poured into ice/water (200 mL), and extracted with EA (3×100 mL). The combined organic layer was washed with brine (50 mL), dried over Na₂SO₄, filtered and concentrated in vacuo. The residue was triturated with EA (80 mL) and filtered. The filtrate cake was collected and dried in vacuo to give the title compound (4.20 g, 67% yield) as an off-white solid. ¹H NMR (400 MHz, DMSO-d₆) δ 11.12 (s, 1H), 7.47 (d, J=2.0 Hz, 1H), 7.22 (d, J=8.4 Hz, 1H), 7.10 (d, J=8.4 Hz, 1H), 5.40-5.35 (m, 1H), 2.34 (s, 3H), 2.92-2.88 (m, 1H), 2.71-2.60 (m, 2H), 2.03-1.99 (m, 1H).

### 3-(((tert-butyldimethylsilyl)oxy)methyl)azetidine (Intermediate F)

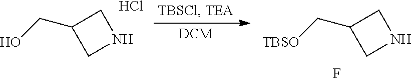

To a solution of azetidin-3-ylmethanol (3.3 g, 26.7 mmol, HCl) in DCM (20 mL) was added TBSCl (4.23 g, 28.0 mmol, 3.44 mL) and TEA (8.11 g, 80.1 mmol, 11.2 mL). The mixture was stirred at 20° C. for 12 hrs. On completion, the mixture was quenched with H₂O (30 mL), extracted with DCM (30 mL×3), dried over anhydrous Na₂SO₄, filtered and concentrated under reduced pressure to give the title compound (5.5 g) as a light yellow oil. LC-MS (ESI⁺) m/z 202.2 (M+H)⁺.

### 1-(1-(2,6-Dioxopiperidin-3-yl)-3-methyl-2-oxo-2,3-dihydro-1H-benzo[d]imidazol-5-yl)azetidine-3-carbaldehyde (Intermediate G)

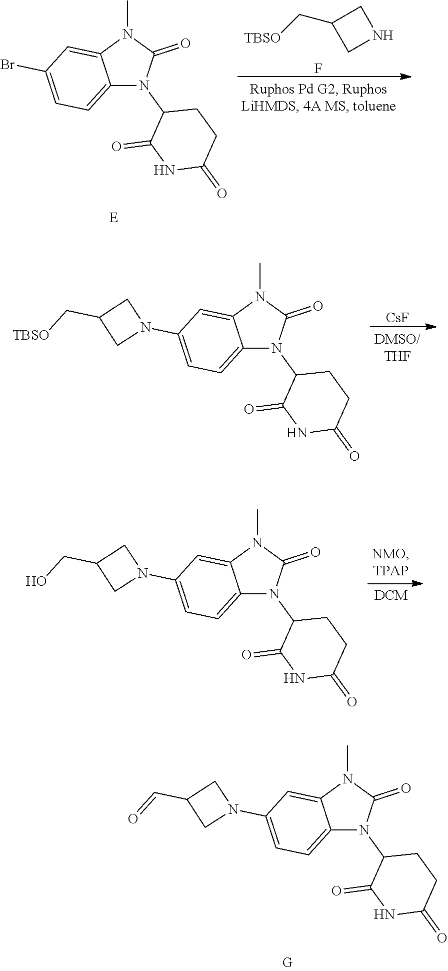

Step 1—3-(5-(3-(((Tert-butyldimethylsilyl)oxy)methyl)azetidin-1-yl)-3-methyl-2-oxo-2,3-dihydro-1H-benzo[d]imidazol-1-yl)piperidine-2,6-dione. To a solution of 3-(5-bromo-3-methyl-2-oxo-benzimidazol-1-yl)piperidine-2,6-dione (2 g, 5.91 mmol, Intermediate E) in toluene (40 mL), was added azetidin-3-ylmethoxy-tert-butyl-dimethyl-silane (1.79 g, 8.87 mmol, Intermediate F), 4 Å molecular sieves (2 g) and LiHMDS (1 M, 29.6 mL). The mixture was purged with N₂ three times, and then RuPhos (276 mg, 591 umol) and [2-(2-aminophenyl)phenyl]-chloro-palladium;dicyclohexyl-[2-(2,6-diisopropoxyphenyl)phenyl]phosphane (459 mg, 591 umol) was added and purged with N₂ three times again. The mixture was stirred at 100° C. for 1 hr under N₂ atmosphere. On completion, the mixture was quenched with FA until the pH=6, filtered and the filtrate was concentrated under reduced pressure to give a residue. The residue was purified by column chromatography (SiO₂, Petroleum ether/Ethyl acetate=1/1 to DCM:EtOAc=1:2) to give the title compound (2.3 g, 81% yield) as a brown solid. LC-MS (ESI⁺) m/z 459.3 (M+H)⁺.

Step 2—3-(5-(3-(hydroxymethyl)azetidin-1-yl)-3-methyl-2-oxo-2,3-dihydro-1H-benzo[d]imidazol-1-yl)piperidine-2,6-dione. To a solution of 3-[5-[3-[[tert-butyl(dimethyl)silyl]oxymethyl]azetidin-1-yl]-3-methyl-2-oxo-benzimidazol-1-yl]piperidine-2,6-dione (2.3 g, 5.01 mmol in DMSO (4 mL) and THF (12 mL) was added CsF (1.52 g, 10.0 mmol, 370 uL). The mixture was stirred at 40° C. for 12 hrs. On completion, the mixture was concentrated under reduced pressure to give a residue. The residue was purified by column chromatography (SiO₂, Petroleum ether/Ethyl acetate=0/1 to MeCN/DCM=1/1) to give the title compound (1.1 g, 62% yield) as a white solid. LC-MS (ESI⁺) m/z 344.8 (M+H)⁺.

Step 3—1-(1-(2,6-dioxopiperidin-3-yl)-3-methyl-2-oxo-2,3-dihydro-1H-benzo[d]imidazol-5-yl)azetidine-3-carbaldehyde. To a solution of 3-[5-[3-(hydroxymethyl)azetidin-1-yl]-3-methyl-2-oxo-benzimidazol-1-yl]piperidine-2,6-dione (300 mg, 871 umol) in DCM (2 mL) and DMSO (1 mL) was added 4 Å molecular sieves (300 mg). Then NMO (204 mg, 1.75 mmol) and oxido(trioxo)ruthenium; tetrapropylammonium (122 mg, 348 umol) was added at 0° C. and the mixture was stirred at 20° C. for 15 min. On completion, the mixture was filtered and washed with THF (20 mL). The filtrate was concentrated under reduced pressure to give DMSO solution of the title compound (230 mg) as a dark liquid. LC-MS (ESI⁺) m/z 360.1 (M+18)⁺.

### 2′-(2-(4-(Piperazin-1-yl)phenyl)pyrimidin-4-yl)-5′,6′-dihydrospiro[cyclopropane-1,7′-pyrrolo[3,2-c]pyridin]-4′(1′H)-one (Intermediate H)

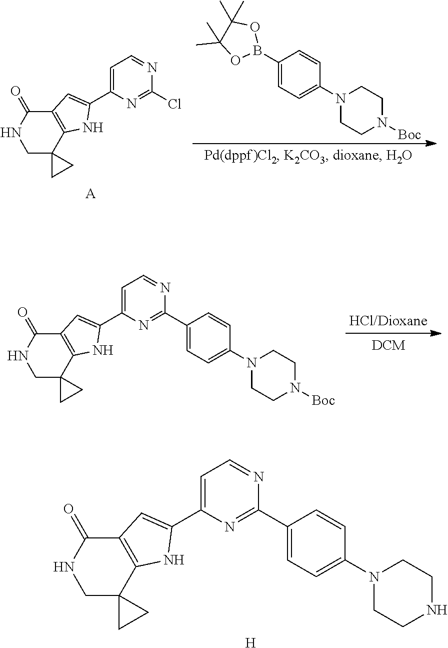

Step 1—Tert-butyl 4-(4-(4-(4-oxo-4,5,6,7-tetrahydro-1H-pyrrolo[3,2-c]pyridin-2-yl)pyrimidin-2-yl)phenyl)piperazine-1-carboxylate. To a solution of 2′-(2-chloropyrimidin-4-yl)-5′,6′-dihydrospiro[cyclopropane-1,7-pyrrolo[3,2-c]pyridin]-4′(1′H)-one (1.5 g, 5.46 mmol, Intermediate A) and tert-butyl 4-(4-(4,4,5,5-tetramethyl-1,3,2-dioxaborolan-2-yl)phenyl)piperazine-1-carboxylate (2.12 g, 5.46 mmol, CAS #70478-90-1) in dioxane (15 mL) and H₂O (3 mL) was added K₂CO₃ (2.26 g, 16.4 mmol) and Pd(dppf)Cl₂ (400 mg, 546 umol) at 25° C. Then the mixture was stirred at 80° C. for 2 hrs. On completion, the reaction mixture was filtered and concentrated in vacuo to give the crude residue. The residue was purified by column chromatography (SiO₂, Petroleum ether/Ethyl acetate=1/1 to DCM:MeOH=10/1) to give the title compound (600 mg, 18% yield) as a yellow solid. LC-MS (ESI⁺) m/z 501.3. (M+H)⁺.

Step 2—2′-(2-(4-(Piperazin-1-yl)phenyl)pyrimidin-4-yl)-5′,6′-dihydrospiro[cyclopropane-1,7′-pyrrolo[3,2-c]pyridin]-4′(1′H)-one. To a solution of 2′-(2-(4-(4-methylpiperazin-1-yl)phenyl)pyrimidin-4-yl)-5′,6′-dihydrospiro[cyclopropane-1,7′-pyrrolo[3,2-c]pyridin]-4′(1′H)-one (100 mg, 200 umol) in DCM (8 mL) was added HCl/dioxane (4 M, 2 mL) at 25° C., then the mixture was stirred at 25° C. for 2 hrs. On completion, the reaction mixture was filtered and concentrated in vacuo to give the title compound (150 mg, HCl) as a yellow solid. LC-MS (ESI⁺) m/z 401.2. (M+H)⁺.

### Tert-butyl 4-(6-methyl-5-(4,4,5,5-tetramethyl-1,3,2-dioxaborolan-2-yl)pyridin-2-yl)piperazine-1-carboxylate (Intermediate I)

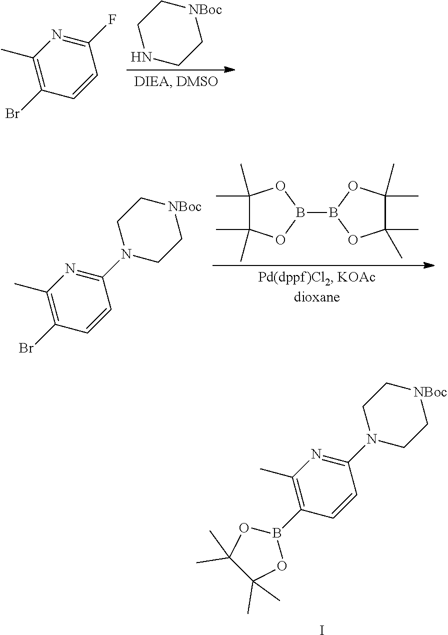

Step 1—Tert-butyl 4-(5-bromo-6-methylpyridin-2-yl)piperazine-1-carboxylate. To a solution of 3-bromo-6-fluoro-2-methyl-pyridine (5 g, 26.3 mmol, CAS #375368-83-5) and tert-butyl piperazine-1-carboxylate (4.90 g, 26.3 mmol) in DMSO (100 mL) was added DIEA (6.80 g, 52.6 mmol). The mixture was then stirred at 120° C. for 12 hrs. On completion, the reaction mixture was filtered and the filter cake was concentrated under reduced pressure to give the title compound (6.7 g) as a white solid. LC-MS (ESI⁺) m/z 357.9 (M+H)⁺.

Step 2—Tert-butyl 4-(6-methyl-5-(4,4,5,5-tetramethyl-1,3,2-dioxaborolan-2-yl)pyridin-2-yl)piperazine-1-carboxylate. A mixture of tert-butyl 4-(5-bromo-6-methyl-2-pyridyl)piperazine-1-carboxylate (3 g, 8.42 mmol), 4,4,5,5-tetramethyl-2-(4,4,5,5-tetramethyl-1,3,2-dioxaborolan-2-yl)-1,3,2-dioxaborolane (4.28 g, 16.8 mmol), Pd(dppf)Cl₂ (616 mg, 842 umol), KOAc (2.48 g, 25.3 mmol) in dioxane (50 mL) was degassed and purged with N₂ three times. Then the mixture was stirred at 80° C. for 2 hrs under N₂ atmosphere. On completion, the reaction mixture was diluted with H₂O (50 mL) and extracted with ethyl acetate (60 mL×3). The combined organic phase was washed with brine 120 mL (40 mL×3), dried over Na₂SO₄, filtered and the filtrate was concentrated under reduced pressure to give a residue. The residue was purified by column chromatography (SiO₂, Petroleum ether/Ethyl acetate=1/0 to 10/1). to give the title compound (1.3 g, 36% yield) as a white solid. LC-MS (ESI⁺) m/z 404.2 (M+H)⁺.

### (15R)-15-methyl-5-(6-piperazin-1-yl-3-pyridyl)-11-thia-6,14,17-triazatetracyclo[8.8.0.0^{2,7}.0^{12,18}]octadeca-1,3,5,7,9,12(18)-hexaen-13-one (Intermediate J)

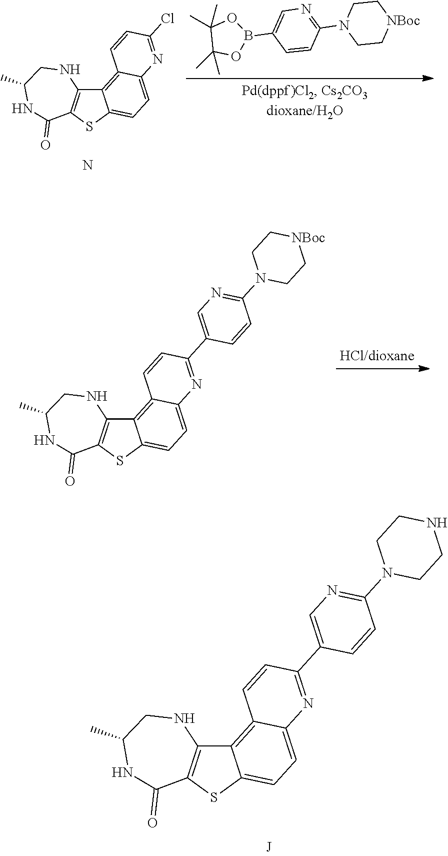

Step 1—Tert-butyl (R)-4-(5-(10-methyl-8-oxo-9,10,11,12-tetrahydro-8H-[1,4]diazepino[5′,6′:4,5]thieno[3,2-f]quinolin-3-yl)pyridin-2-yl)piperazine-1-carboxylate. A mixture of (R)-3-chloro-10-methyl-9,10,11,12-tetrahydro-8H-[1,4]diazepino[5′,6′:4,5]thieno[3,2-f]quinolin-8-one (400 mg, 1.26 mmol, Intermediate N), tert-butyl 4-(5-(4,4,5,5-tetramethyl-1,3,2-dioxaborolan-2-yl)pyridin-2-yl)piperazine-1-carboxylate (735 mg, 1.89 mmol, CAS #496786-98-2), Pd(dppf)Cl₂ (92.1 mg, 156 umol) and K₂CO₃ (522 mg, 3.78 mmol) in dioxane (3 mL) and H₂O (1 mL) was degassed and purged with N₂ three times. Then the mixture was stirred at 80° C. for 2 hrs under N₂ atmosphere. The reaction mixture was diluted with H₂O (5 mL) and extracted with EA (5 mL×3). The combined organic layers were dried over Na₂SO₄, filtered and the filtrate was concentrated under reduced pressure to give a residue. The residue was purified by column chromatography (SiO₂, DCM:MeOH=10:1) to give the title compound (550 mg, 72% yield) as a yellow solid. LC-MS (ESI⁺) m/z 545.4 (M+H)⁺.

Step 2—(R)-10-methyl-3-(6-(piperazin-1-yl)pyridin-3-yl)-9,10,11,12-tetrahydro-8H-[1,4]diazepino[5′,6′:4,5]thieno[3,2-f]quinolin-8-one. To a solution of tert-butyl 4-[5-[(15R)-15-methyl-13-oxo-11-thia-6,14,17-triazatetracyclo[8.8.0.02,7.012,18]octadeca-1,3,5,7,9,12(18)-hexaen-5-yl]-2-pyridyl]piperidine-1-carboxylate (250 mg, 459 umol) in DCM (3 mL) was added HCl/dioxane (4 M, 1 mL), then the mixture was stirred at 25° C. for 2 hrs. On completion, the reaction mixture was concentrated under reduced pressure to give the title compound (250 mg) as a yellow solid. LC-MS (ESI⁺) m/z 445.4 (M+H)⁺.

### 2′-(2-chloropyrimidin-4-yl)-5′,6′-dihydrospiro[cyclopentane-1,7′-pyrrolo[3,2-c]pyridin]-4′(1′H)-one (Intermediate K)

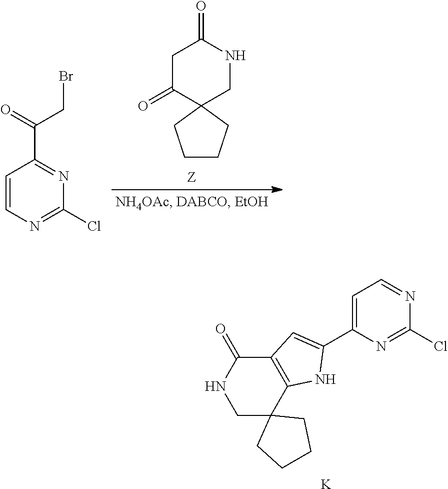

To a solution of 7-azaspiro[4.5]decane-8,10-dione (1.42 g, 8.49 mmol, Intermediate Z) in EtOH (20 mL) was added 2-bromo-1-(2-chloropyrimidin-4-yl)ethanone (3 g, 12.7 mmol, synthesized via Steps 1-2 of Intermediate A), DABCO (953 mg, 8.49 mmol, 934 uL) and NH₄OAc (9.82 g, 127 mmol). The mixture was stirred at 50° C. for 1 hr. On completion, the reaction mixture was quenched with H₂O (40 mL) at 25° C., and then extracted with EA (50 mL×4). The combined organic layers were washed with brine (50 mL×2), dried over Na₂SO₄, filtered and concentrated under reduced pressure to give a residue. The residue was purified by column chromatography (SiO2, Petroleum ether/Ethyl acetate=0/1 to DCM:MeOH=20:1) to give a title compound (600 mg mmol, 15% yield) as an orange solid. LC-MS (ESI⁺) m/z 303.1 (M+H)⁺.

### 2′-(2-(4-(piperazin-1-yl)phenyl)pyrimidin-4-yl)-5′,6′-dihydrospiro[cyclopentane-1,7′-pyrrolo[3,2-c]pyridin]-4′(1′H)-one (Intermediate L)

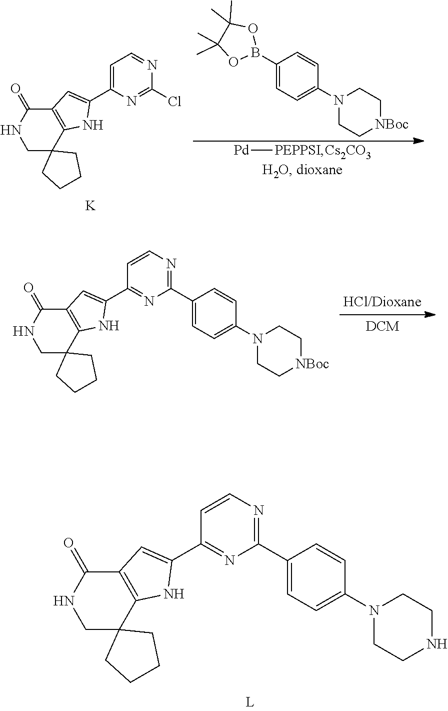

Step 1—Tert-butyl 4-(4-(4-(4′-oxo-1′,4′,5′,6′-tetrahydrospiro[cyclopentane-1,7′-pyrrolo[3,2-c]pyridin]-2′-yl)pyrimidin-2-yl)phenyl)piperazine-1-carboxylate. To a solution of 2-(2-chloropyrimidin-4-yl)spiro[5,6-dihydro-1H-pyrrolo[3,2-c]pyridine-7,1′-cyclopentane]-4-one (200 mg, 661 umol, Intermediate K) in dioxane (2 mL) and H₂O (0.5 mL) was added tert-butyl 4-[4-(4,4,5,5-tetramethyl-1,3,2-dioxaborolan-2-yl)phenyl]piperazine-1-carboxylate (308 mg, 793 umol, CAS #470478-90-1), 1,3-bis[2,6-bis(1-propylbutyl)phenyl]-4,5-dichloro-2H-imidazol-1-ium-2-ide;3-chloropyridine; dichloropalladium (64.3 mg, 66.1 umol) and Cs₂CO₃ (430 mg, 1.32 mmol). The mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=5/1 to DCM:MeOH=20:1) to give a title compound (200 mg, 47% yield) as a yellow solid. LC-MS (ESI⁺) m/z 529.2 (M+H)⁺.

Step 2—2′-(2-(4-(Piperazin-1-yl)phenyl)pyrimidin-4-yl)-5′,6′-dihydrospiro[cyclopentane-1,7′-pyrrolo[3,2-c]pyridin]-4′(1′H)-one. To a solution of tert-butyl 4-[4-[4-(4-oxospiro[5,6-dihydro-1H-pyrrolo[3,2-c]pyridine-7,1′-cyclopentane]-2-yl)pyrimidin-2-yl]phenyl]piperazine-1-carboxylate (200 mg, 378 umol) in DCM (4 mL) was added HCl/dioxane (4 M, 94.6 uL). The reaction was stirred at 25° C. for 2 hr. On completion, the reaction mixture was concentrated under reduced pressure to give the title compound. LC-MS (ESI⁺) m/z 429.0 (M+H)⁺.

### 2′-(2-(2-fluoro-4-(piperazin-1-yl)phenyl)pyrimidin-4-yl)-5′,6′-dihydrospiro[cyclopentane-1,7′-pyrrolo[3,2-c]pyridin]-4′(1′H)-one (Intermediate M)

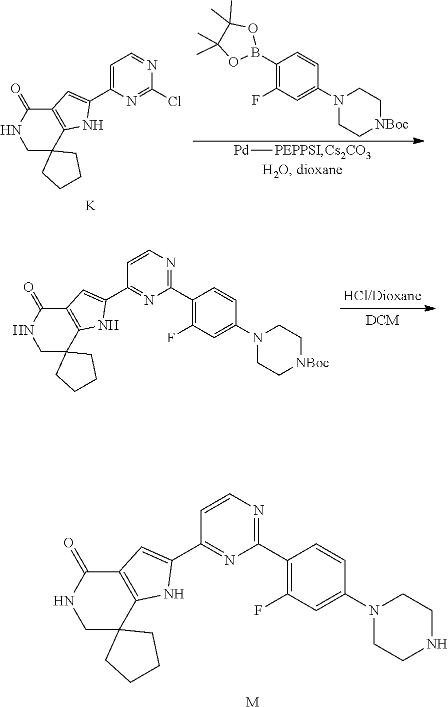

Step 1—Tert-butyl 4-(3-fluoro-4-(4-(4′-oxo-1′,4′,5′,6′-tetrahydrospiro[cyclopentane-1,7′-pyrrolo[3,2-c]pyridin]-2′-yl)pyrimidin-2-yl)phenyl)piperazine-1-carboxylate. To a solution of 2′-(2-chloropyrimidin-4-yl)-5′,6′-dihydrospiro[cyclopentane-1,7′-pyrrolo[3,2-c]pyridin]-4′(1′H)-one (300 mg, 991 umol, Intermediate K) in dioxane (2 mL) and H₂O (0.5 mL) was added tert-butyl 4-(3-fluoro-4-(4,4,5,5-tetramethyl-1,3,2-dioxaborolan-2-yl)phenyl)piperazine-1-carboxylate (483 mg, 1.19 mmol, CAS #1146950-53-9), 1,3-bis[2,6-bis(1-propylbutyl)phenyl]-4,5-dichloro-2H-imidazol-1-ium-2-ide;3-chloropyridine; dichloropalladium (96.4 mg, 99.1 umol) and Cs₂CO₃ (646 mg, 1.98 mmol). The mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=1/1 to DCM:MeOH=20/1) to give the title compound (280 mg, 37% yield) as a yellow solid. LC-MS (ESI⁺) m/z 547.2 (M+H)⁺.

Step 2—2′-(2-(2-Fluoro-4-(piperazin-1-yl)phenyl)pyrimidin-4-yl)-5′,6′-dihydrospiro[cyclopentane-1,7′-pyrrolo[3,2-c]pyridin]-4′(1′H)-one. To a solution of tert-butyl 4-(3-fluoro-4-(4-(4′-oxo-1′,4′,5′,6′-tetrahydrospiro[cyclopentane-1,7′-pyrrolo[3,2-c]pyridin]-2′-yl)pyrimidin-2-yl)phenyl)piperazine-1-carboxylate (280 mg, 512 umol) in DCM (4 mL) was added HCl/dioxane (4 M, 2 mL). The reaction was stirred at 25° C. for 1 hr. On completion, the reaction mixture was concentrated under reduced pressure to give the title compound (200 mg, HCl) as a red solid. LC-MS (ESI⁺) m/z 447.2 (M+H)⁺.

### (R)-3-chloro-10-methyl-9,10,11,12-tetrahydro-8H-[1,4]diazepino[5′,6′:4,5]thieno[3,2-f]quinolin-8-one (Intermediate N)

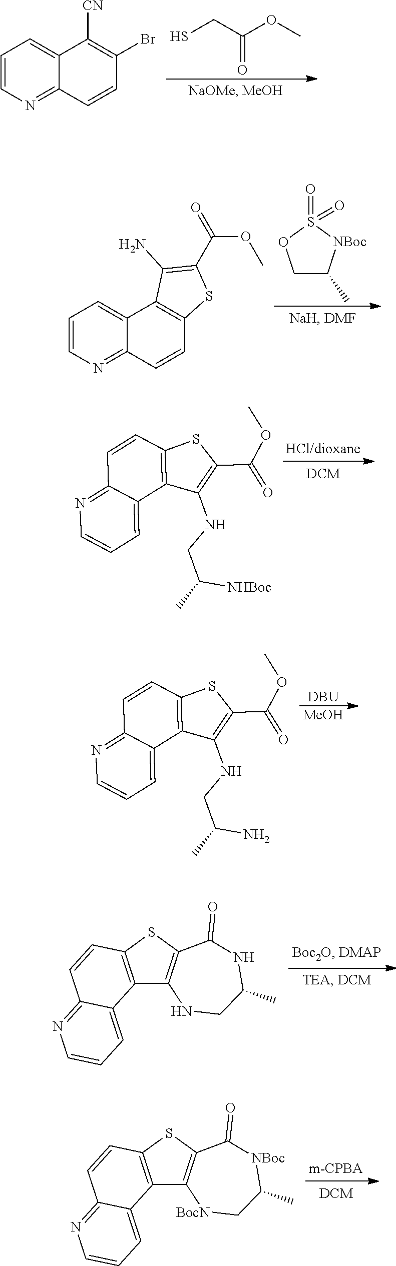

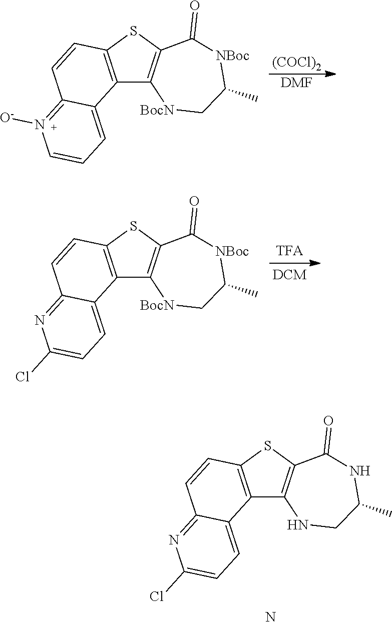

Step 1—Methyl 1-aminothieno[3,2-f]quinoline-2-carboxylate. To a solution of 6-bromoquinoline-5-carbonitrile (500 mg, 2.15 mmol, CAS #1188365-70-9) in MeOH (5 mL) was added NaOMe (5 M, 858 uL) and methyl 2-mercaptoacetate (455 mg, 4.29 mmol, 389 uL). The mixture was stirred at 90° C. for 12 hrs. On completion, the mixture was concentrated under reduced pressure, then diluted with water (5 mL), and filtered to give the title compound (180 mg) as a yellow solid. LC-MS (ESI⁺) m/z 259.1 (M+H)⁺.

Step 2—(R)-methyl 1-((2-((tert-butoxycarbonyl)amino)propyl)amino)thieno[3,2-f]quinoline-2-carboxylate. To a solution of methyl 1-aminothieno[3,2-f]quinoline-2-carboxylate (10 g, 38.7 mmol) in DMF (100 mL) was added NaH (1.55 g, 38.7 mmol, 60% dispersion in mineral oil). The mixture was stirred at 0° C. for 0.5 hr. Then (R)-tert-butyl 4-methyl-1,2,3-oxathiazolidine-3-carboxylate 2,2-dioxide (10.1 g, 42.6 mmol, CAS #454248-53-4) was added, and the mixture was stirred at 0° C. for 0.5 hr. On completion, the mixture was quenched with 0.5 M HCl solution (500 mL). Then aqueous Na₂CO₃ solution was added to the mixture until pH=8 and solid formed. The solid was filtered and dried to give the title compound (8 g) as a brown solid. LC-MS (ESI⁺) m/z 416.4 (M+H)⁺.

Step 3—(R)-methyl 1-((2-aminopropyl)amino)thieno[3,2-f]quinoline-2-carboxylate. To a solution of (R)-methyl 1-((2-((tert-butoxycarbonyl)amino)propyl)amino)thieno[3,2-f]quinoline-2-carboxylate (10 g, 24.1 mmol) in DCM (100 mL) was added HCl/dioxane (4 M, 25 mL). The mixture was stirred at 20° C. for 2 hrs. On completion, the mixture was filtered and the cake was washed with DCM (100 mL). Then the solid was dried under reduced pressure to give the title compound (8.3 g) as a brown solid. LC-MS (ESI⁺) m/z 316.1 (M+H)⁺.

Step 4—(R)-10-methyl-9,10,11,12-tetrahydro-8H-[1,4]diazepino[5′,6′:4,5]thieno[3,2-f]quinolin-8-one. To a solution of (R)-methyl 1-((2-aminopropyl)amino)thieno[3,2-f]quinoline-2-carboxylate (8.3 g, 18.6 mmol, HCl salt) in MeOH (100 mL) was added DBU (14.2 g, 93.2 mmol). The mixture was stirred at 70° C. for 12 hrs. On completion, the mixture was concentrated under reduced pressure to give a residue. Then 1M HCl solution was added until pH<5, followed by aqueous NaHCO₃ solution until pH=8, The mixture was then extracted with DCM (100 mL×3) and washed with H₂O (100 mL). The organic phase was concentrated under reduced pressure to give the title compound (4.8 g) as a brown solid. LC-MS (ESI⁺) m/z 283.9 (M+H)⁺.

Step 5—(R)-di-tert-butyl 10-methyl-8-oxo-10,11-dihydro-8H-[1,4]diazepino[5′,6′:4,5]thieno[3,2-f]quinoline-9,12-dicarboxylate. To a solution of (R)-10-methyl-9,10,11,12-tetrahydro-8H-[1,4]diazepino[5′,6′:4,5]thieno[3,2-f]quinolin-8-one (4.8 g, 14.1 mmol) in DCM (100 mL) was added DMAP (0.5 g, 4.09 mmol) and TEA (4.27 g, 42.2 mmol, 5.87 mL). The mixture was stirred at 25° C. for 0.2 hr, then Boc₂O (12.3 g, 56.2 mmol, 12.9 mL) was added and the mixture was stirred at 25° C. for 3.8 hr. On completion, the mixture was washed with H₂O (100 mL), then the organic layer was concentrated under reduced pressure to give the title compound (7 g) as a pink solid. LC-MS (ESI⁺) m/z 484.3 (M+H)⁺.

Step 6—(R)-9,12-bis(tert-butoxycarbonyl)-10-methyl-8-oxo-9,10,11,12-tetrahydro-8H-[1,4]diazepino[5′,6′:4,5]thieno[3,2-f]quinoline 4-oxide. To a solution of (R)-di-tert-butyl 10-methyl-8-oxo-10,11-dihydro-8H-[1,4]diazepino[5′,6′:4,5]thieno[3,2-f]quinoline-9,12-dicarboxylate (7 g, 11.6 mmol) in DCM (200 mL) was added m-CPBA (3 g, 17.4 mmol) slowly at 0° C. Then the mixture was stirred at 30° C. for 3 hrs. On completion, the mixture was washed with H₂O (100 mL), then washed with NaHCO₃ solution (100 mL×3). The organic phase was dried over anhydrous Na₂SO₄, filtered and the solution was concentrated under reduced pressure to give the title compound (6.4 g) as a yellow solid. LC-MS (ESI⁺) m/z 500.0 (M+H)⁺.

Step 7—(R)-di-tert-butyl 3-chloro-10-methyl-8-oxo-10,11-dihydro-8H-[1,4]diazepino[5′,6′:4,5]thieno[3,2-f]quinoline-9,12-dicarboxylate. To a solution of (R)-9,12-bis(tert-butoxycarbonyl)-10-methyl-8-oxo-9,10,11,12-tetrahydro-8H-[1,4]diazepino[5′,6′:4,5]thieno[3,2-f]quinoline 4-oxide (10),2,4,6,8,12(18)-hexaene-14,17-dicarboxylate (3 g, 5.40 mmol) in DMF (70 mL) was added (COCl)₂ (1.03 g, 8.11 mmol) dropwise at 0° C. The mixture was stirred at 20° C. for 4 hrs. On completion, (COCl)₂ was removed under reduced pressure, then diluted with H₂O (200 mL) and stirred at 20° C. for 10 mins until a solid formed. The mixture was filtered to give a cake, washed with H₂O, then cake was dried to give the title compound (2.2 g) as a yellow solid. LC-MS (ESI⁺) m/z 518.2 (M+H)⁺.

Step 8—(R)-3-chloro-10-methyl-9,10,11,12-tetrahydro-8H-[1,4]diazepino[5′,6′:4,5]thieno[3,2-f]quinolin-8-one. To a solution of (R)-di-tert-butyl 3-chloro-10-methyl-8-oxo-10,11-dihydro-8H-[1,4]diazepino[5′,6′:4,5]thieno[3,2-f]quinoline-9,12-dicarboxylate (1.99 g, 3.85 mmol) in DCM (30 mL) was added TFA (15.4 g, 135 mmol, 10 mL). The mixture was stirred at 20° C. for 3 hrs. On completion, the mixture was concentrated under reduced pressure and quenched with NaHCO₃ solution until pH=8. Then the mixture was extracted with DCM (100 mL×3), dried over anhydrous Na₂SO₄, filtered and concentrated under reduced pressure to give the title compound (1 g) as a yellow solid. LC-MS (ESI⁺) m/z 318.0 (M+H)⁺.

### (R)-3-(6-fluoropyridin-3-yl)-10-methyl-9,10,11,12-tetrahydro-8H-[1,4]diazepino[5′,6′:4,5]thieno[3,2-f]quinolin-8-one (Intermediate O)

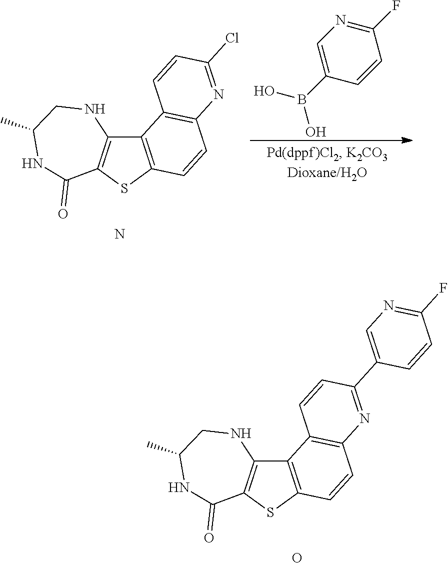

To a solution of (15R)-5-chloro-15-methyl-11-thia-6,14,17-triazatetracyclo[8.8.0.0^{2,7}.0^{12,18}]octadeca-1(10),2(7),3,5,8,12(18)-hexaen-13-one (1 g, 3.15 mmol, Intermediate N) in dioxane (30 mL) and H₂O (8 mL) was added (6-fluoro-3-pyridyl)boronic acid (665 mg, 4.72 mmol, CAS #351019-18-6), Pd(dppf)Cl₂ (230 mg, 315 umol) and K₂CO₃ (1.30 g, 9.44 mmol). The reaction was degassed and purged with N₂ three times, and then the mixture was stirred at 80° C. for 2 hrs under N₂ atmosphere. On completion, the reaction mixture was quenched with H₂O (200 mL) at 25° C., and then filtered and concentrated under reduced pressure to give the crude product. The crude product was triturated with EA at 25° C. for 10 min, then filtered and dried to give the title compound (950 mg, 74% yield) as a gray solid. LC-MS (ESI⁺) m/z 379.1 (M+H)⁺.

### 3-(3-methyl-2-oxo-5-(2,7-diazaspiro[3.5]nonan-7-yl)-2,3-dihydro-1H-benzo[d]imidazol-1-yl)piperidine-2,6-dione (Intermediate P)

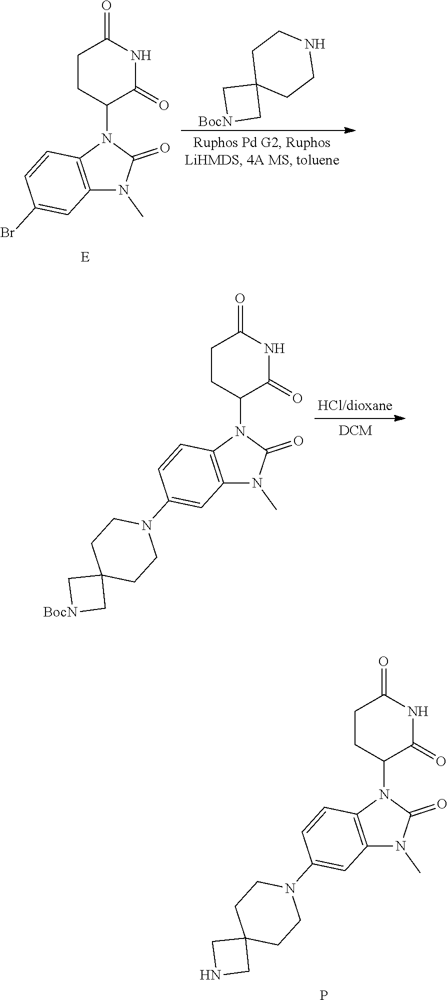

Step 1—Tert-butyl 7-(1-(2,6-dioxopiperidin-3-yl)-3-methyl-2-oxo-2,3-dihydro-1H-benzo[d]imidazol-5-yl)-2,7-diazaspiro[3.5]nonane-2-carboxylate. To a solution of 3-(5-bromo-3-methyl-2-oxo-benzimidazol-1-yl)piperidine-2,6-dione (3 g, 8.87 mmol, Intermediate E), 4 Å molecular sieves (2 g) and tert-butyl 2,7-diazaspiro[3.5]nonane-2-carboxylate (2.11 g, 9.32 mmol, CAS #236406-55-6) in toluene (60 mL), was added LiHMDS (1 M, 53.2 mL) at 0° C. and the mixture was purged with N₂ three times. Then RuPhos (828 mg, 1.77 mmol) and [2-(2-aminophenyl)phenyl]-methylsulfonyloxy-palladium;dicyclohexyl-[2-(2,6-diisopropoxyphenyl)phenyl]phosphane (1.48 g, 1.77 mmol) was added and the mixture was purged with N₂ three times, then the mixture was stirred at 100° C. for 1 hr. On completion, the reaction mixture was quenched with FA at 0° C. until pH=7, and then filtered and concentrated under reduced pressure to give a residue. The residue was purified by column chromatography (SiO₂, Petroleum ether/Ethyl acetate=10/1 to 0/1, THF:EA=10:1) to give the title compound (2.6 g, 50% yield) as a gray solid. LC-MS (ESI⁺) m/z 484.3 (M+H)⁺.

Step 2—3-(3-Methyl-2-oxo-5-(2,7-diazaspiro[3.5]nonan-7-yl)-2,3-dihydro-1H-benzo[d]imidazol-1-yl)piperidine-2,6-dione. To a solution of tert-butyl 7-[1-(2,6-dioxo-3-piperidyl)-3-methyl-2-oxo-benzimidazol-5-yl]-2,7-diazaspiro[3.5]nonane-2-carboxylate (300 mg, 620 umol) in DCM (15 mL) was added HCl/dioxane (4 M, 1 mL). The mixture was stirred at 25° C. for 1 hr. On completion, the reaction mixture was filtered and the filtrate was concentrated under reduced pressure to give the crude product. The crude product was triturated with DCM at 25° C. for 10 min, then filtered and dried to give the title compound (190 mg, 66% yield, FA) as a white solid. LC-MS (ESI⁺) m/z 384.4 (M+H)⁺.

### 3-(3-Methyl-2-oxo-5-(2,8-diazaspiro[4.5]decan-8-yl)-2,3-dihydro-1H-benzo[d]imidazol-1-yl)piperidine-2,6-dione (Intermediate Q)

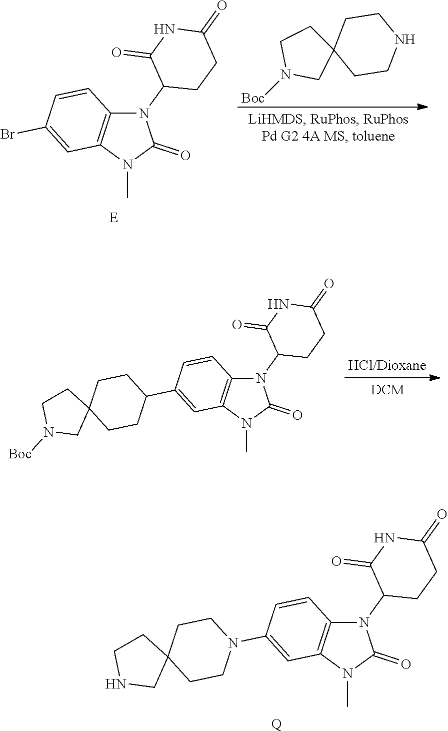

Step 1—Tert-butyl 8-(1-(2,6-dioxopiperidin-3-yl)-3-methyl-2-oxo-2,3-dihydro-1H-benzo[d]imidazol-5-yl)-2,8-diazaspiro[4.5]decane-2-carboxylate. To a solution of 3-(5-bromo-3-methyl-2-oxo-2,3-dihydro-1H-benzo[d]imidazol-1-yl)piperidine-2,6-dione (500 mg, 1.48 mmol, Intermediate E) and tert-butyl 2,8-diazaspiro[4.5]decane-2-carboxylate (426 mg, 1.77 mmol, CAS #1180509-95-8) in toluene (15 mL) was added RuPhos (34.5 mg, 73.9 umol), [2-(2-aminophenyl)phenyl]-chloro-palladium dicyclohexyl-[2-(2,6-diisopropoxyphenyl)phenyl]phosphane (57.4 mg, 73.9 umol), 4 Å molecular sieves (50 mg, 1.48 mmol) and LiHMDS (1 M, 8.87 mL). The mixture was stirred at 80° C. for 2 hrs under N₂ atmosphere. The reaction mixture was quenched with FA (8 mL) at 0° C., and then diluted with H₂O (20 mL) and extracted with EA (20 mL×3). The combined organic layers were dried over Na₂SO₄, filtered and concentrated under reduced pressure to give the title compound (600 mg) as a brown solid. LC-MS (ESI⁺) m/z 498.0 (M+1)⁺.

Step 2—3-(3-Methyl-2-oxo-5-(2,8-diazaspiro[4.5]decan-8-yl)-2,3-dihydro-1H-benzo[d]imidazol-1-yl)piperidine-2,6-dione. To a solution of tert-butyl 8-(1-(2,6-dioxopiperidin-3-yl)-3-methyl-2-oxo-2,3-dihydro-1H-benzo[d]imidazol-5-yl)-2,8-diazaspiro[4.5]decane-2-carboxylate (250 mg, 502 umol) in DCM (8 mL) was added HCl/dioxane (4 M, 2 mL). The mixture was stirred at 25° C. for 0.5 hr. On completion, the reaction mixture was washed with DCM (15 mL×4), filtered under reduced pressure to give the title compound (210 mg, HCl) as a red solid. LC-MS (ESI⁺) m/z 398.0 (M+1)⁺.

### (R)-10-methyl-3-(4-oxopiperidin-1-yl)-9,10,11,12-tetrahydro-8H-[1,4]diazepino[5′,6′:4,5]thieno[3,2-f]quinolin-8-one (Intermediate R)

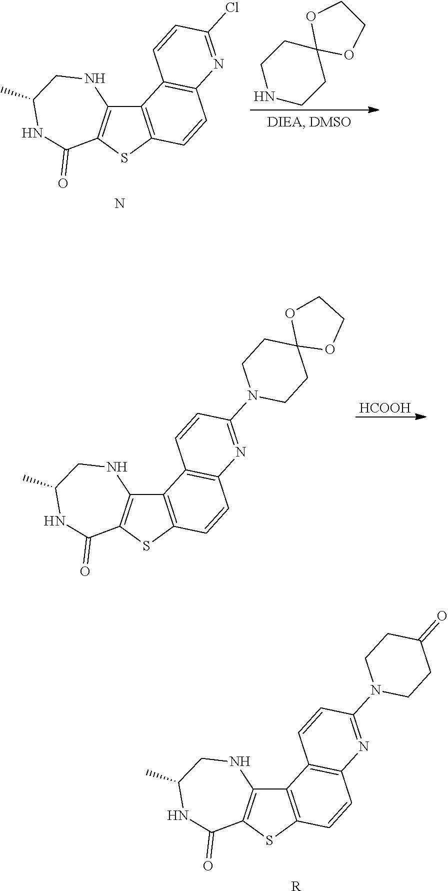

Step 1—(R)-10-methyl-3-(1,4-dioxa-8-azaspiro[4.5]decan-8-yl)-9,10,11,12-tetrahydro-8H-[1,4]diazepino[5′,6′:4,5]thieno[3,2-f]quinolin-8-one. To a solution of (R)-3-chloro-10-methyl-9,10,11,12-tetrahydro-8H-[1,4]diazepino[5′,6′:4,5]thieno[3,2-f]quinolin-8-one (1 g, 3.15 mmol, Intermediate N) in DMSO (20 mL) was added DIEA (4.07 g, 31.5 mmol) and 1,4-dioxa-8-azaspiro[4.5]decane (901 mg, 6.29 mmol, CAS #177-11-7). The mixture was stirred at 120° C. for 48 hrs. On completion, the reaction mixture was poured into H₂O (50 mL) and stirred at 20° C. for 10 min, then filtered under reduced pressure to give the title compound (1 g) as a brown solid. LC-MS (ESI⁺) m/z 425.2 (M+H)⁺.

Step 2—(R)-10-methyl-3-(4-oxopiperidin-1-yl)-9,10,11,12-tetrahydro-8H-[1,4]diazepino[5′,6′:4,5]thieno[3,2-f]quinolin-8-one. (R)-10-methyl-3-(1,4-dioxa-8-azaspiro[4.5]decan-8-yl)-9,10,11,12-tetrahydro-8H-[1,4]diazepino[5′,6′:4,5]thieno[3,2-f]quinolin-8-one (100 mg, 236 umol) was added to HCOOH (2 mL). The mixture was stirred at 20° C. for 3 hrs. On completion, the reaction mixture was concentrated under reduced pressure to give the title compound (85 mg) as a brown gum. LC-MS (ESI⁺) m/z 381.1 (M+H)⁺.

### 2-(5-methoxy-1-(1-(4-methoxybenzyl)-2,6-dioxopiperidin-3-yl)-3-methyl-2-oxo-2,3-dihydro-1H-benzo[d]imidazol-4-yl)acetaldehyde (Intermediate S)

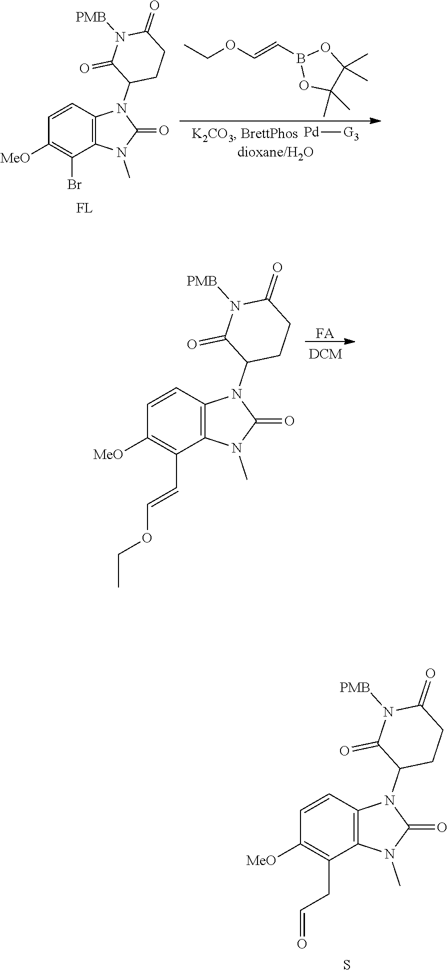

Step 1—(E)-3-(4-(2-ethoxyvinyl)-5-methoxy-3-methyl-2-oxo-2,3-dihydro-1H-benzo[d]imidazol-1-yl)-1-(4-methoxybenzyl)piperidine-2,6-dione. A mixture of 3-(4-bromo-5-methoxy-3-methyl-2-oxo-benzimidazol-1-yl)-1-[(4-methoxyphenyl) methyl]piperidine-2,6-dione (300 mg, 614 umol, Intermediate FL), 2-[(E)-2-ethoxyvinyl]-4,4,5,5-tetramethyl-1,3,2-dioxaborolane (243 mg, 1.23 mmol), K₂CO₃ (255 mg, 1.84 mmol), and BrettPhos Pd G3 (33.4 mg, 36.9 umol) in dioxane (6 mL) and H₂O (1.5 mL) was degassed and purged with N₂ three times. Then the mixture was stirred at 50° C. for 2 hrs under N₂ atmosphere. On completion, the reaction mixture was concentrated under reduced pressure to remove solvents. The residue was purified by column chromatography (SiO₂, Petroleum ether/Ethyl acetate=1/0 to 1/1) to give the title compound (147 mg, 37% yield) as a light yellow solid. LC-MS (ESI⁺) m/z 480.2 (M+H)⁺.

Step 2—2-(5-methoxy-1-(1-(4-methoxybenzyl)-2,6-dioxopiperidin-3-yl)-3-methyl-2-oxo-2,3-dihydro-1H-benzo[d]imidazol-4-yl)acetaldehyde. To a solution of 3-[4-[(E)-2-ethoxyvinyl]-5-methoxy-3-methyl-2-oxo-benzimidazol-1-yl]-1-[(4-methoxyphenyl)methyl]piperidine-2,6-dione (127 mg, 265 umol) in DCM (3 mL) was added FA (1 mL). The mixture was stirred at 40° C. for 2 hrs. On completion, the reaction mixture was concentrated under reduced pressure to give the title compound (130 mg) as a yellow solid. LC-MS (ESI⁺) m/z 452.2 (M+H)⁺.

### 5-Azaspiro[2.5]octane-6,8-dione (Intermediate T)

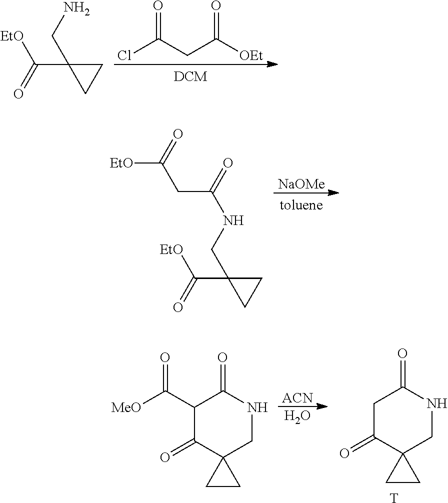

Step 1—Ethyl 1-((3-ethoxy-3-oxopropanamido)methyl)cyclopropanecarboxylate. To a solution of ethyl 1-(aminomethyl)cyclopropanecarboxylate (8 g, 55.9 mmol, CAS #36239-09-5) in DCM (160 mL) was added ethyl 3-chloro-3-oxo-propanoate (8.41 g, 55.9 mmol, 7.01 mL, CAS #400840-72-6) at 0° C. The mixture was stirred at 0° C. for 0.5 hrs. On completion, the reaction mixture was quenched with ice-water (50 mL) at 0° C., the pH of the mixture was adjusted to ˜8 with Na₂CO₃, then extracted with DCM (100 mL×2). The combined organic layers were washed with aqueous NaCl (100 mL×2), dried over Na₂SO₄, filtered and concentrated under reduced pressure to give a residue. The residue was purified by column chromatography (SiO₂, Petroleum ether/Ethyl acetate=10/1 to 3/1, R_{f}=0.1) to give the title compound (9 g, 63% yield) as a yellow oil. LC-MS (ESI⁺) m/z 258.2 (M+H)⁺.

Step 2—Methyl 6,8-dioxo-5-azaspiro[2.5]octane-7-carboxylate. To a solution of ethyl 1-[[(3-ethoxy-3-oxo-propanoyl)amino]methyl]cyclopropanecarboxylate (6 g, 23.3 mmol) in toluene (60 mL) was added NaOMe (5.4 M, 5.84 mL). The mixture was stirred at 80° C. for 1 hr. On completion, the reaction mixture was quenched with ice-water (30 mL) at 0° C., the pH of the mixture was adjusted to about 3-4 with HCl (1 M), then extracted with EtOAc (100 mL×2). The combined organic layers were washed with aqueous NaCl (100 mL×2), dried over Na₂SO₄, filtered and concentrated under reduced pressure to give the title compound (4 g) as yellow solid. LC-MS (ESI⁺) m/z 198.1 (M+H)⁺.

Step 3—5-Azaspiro[2.5]octane-6,8-dione. To a solution of methyl 6,8-dioxo-5-azaspiro[2.5]octane-7-carboxylate (4 g, 20.29 mmol) in MeCN (50 mL) and H₂O (3 mL). The mixture was stirred at 80° C. for 4 hrs. On completion, the reaction mixture was concentrated under reduced pressure to remove solvent to give the title compound (3.2 g) as orange solid. LC-MS (ESI⁺) m/z 140.1 (M+H)⁺.

### 2′-(2-chloropyridin-4-yl)-5′,6′-dihydrospiro[cyclopropane-1,7′-pyrrolo[3,2-c]pyridin]-4′(1′H)-one (Intermediate U)

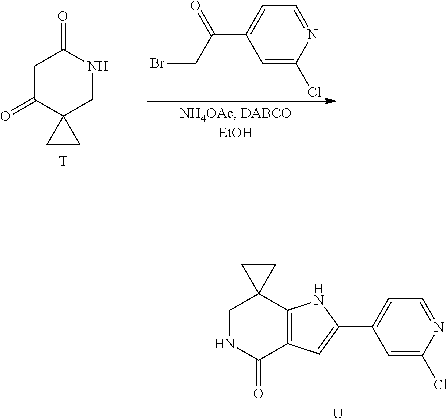

A mixture of 5-azaspiro[2.5]octane-6,8-dione (3 g, 21.6 mmol, Intermediate T), 2-bromo-1-(2-chloro-4-pyridyl)ethanone (10.11 g, 43.1 mmol, CAS #23794-16-3), NH₄OAc (16.6 g, 215 mmol) in EtOH (50 mL) was stirred at 30° C. for 3 hrs. On completion, the reaction mixture was concentrated under reduced pressure to remove solvent. The residue was diluted with DCM (100 mL) and washed with H₂O (100 mL). The combined organic layers were washed with aqueous NaCl (100 mL×2), dried over Na₂SO₄, filtered and concentrated under reduced pressure to give a crude product. The crude product was purified by reversed-phase (0.1% FA condition) to give the title compound (2.3 g, 39% yield, FA) as a yellow solid. ¹H NMR (400 MHz, DMSO-d₆) δ=11.18 (br s, 1H), 8.29-8.24 (m, 1H), 7.79-7.75 (m, 1H), 7.68-7.64 (m, 1H), 7.22-7.09 (m, 2H), 3.27-3.24 (m, 2H), 2.08-2.05 (m, 1H), 1.27-1.22 (m, 2H), 1.02-0.96 (m, 2H) LC-MS (ESI⁺) m/z 274.0 (M+H)⁺.

### 2′-(2-(4-(piperazin-1-yl)phenyl)pyridin-4-yl)-5′,6′-dihydrospiro[cyclopropane-1,7′-pyrrolo[3,2-c]pyridin]-4′(1′H)-one (Intermediate V)

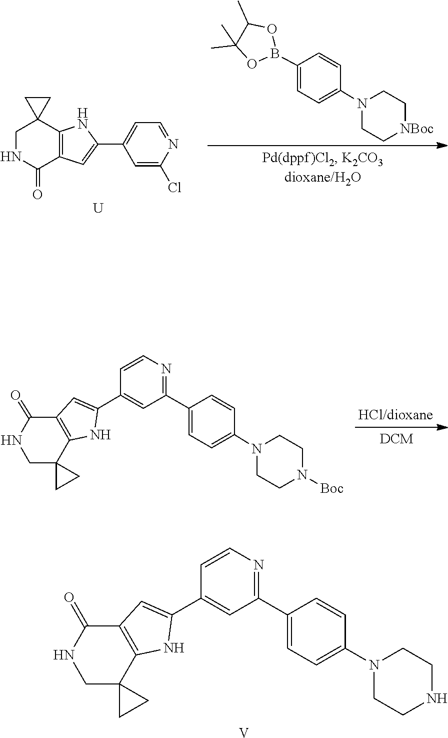

Step 1—Tert-butyl 4-(4-(4-(4′-oxo-1′,4′,5′,6′-tetrahydrospiro[cyclopropane-1,7′-pyrrolo[3,2-c]pyridin]-2′-yl)pyridin-2-yl)phenyl)piperazine-1-carboxylate. A mixture of 2-(2-chloro-4-pyridyl)spiro[5,6-dihydro-1H-pyrrolo[3,2-c]pyridine-7,1′-cyclopropane]-4-one (1 g, 3.65 mmol, Intermediate U), tert-butyl 4-[4-(4,4,5,5-tetramethyl-1,3,2-dioxaborolan-2-yl)phenyl]piperazine-1-carboxylate (2.13 g, 5.48 mmol, CAS #470478-90-1), Pd(dppf)Cl₂.CH₂Cl₂ (298 mg, 365 umol), and Cs₂CO₃ (3.57 g, 11.0 mmol) in dioxane (4 mL) and H₂O (1 mL) was degassed and purged with N₂ three times. Then the mixture was stirred at 100° C. for 2 hrs under N₂ atmosphere. On completion, the reaction mixture was concentrated under reduced pressure to give a residue. The residue was purified by column chromatography (SiO₂, DCM:MeOH=10:1, R_{f}=0.26) to give the title compound (1.1 g) as a white solid. LC-MS (ESI⁺) m/z 500.4 (M+H)⁺.

Step 2—2′-(2-(4-(Piperazin-1-yl)phenyl)pyridin-4-yl)-5′,6′-dihydrospiro[cyclopropane-1,7′-pyrrolo[3,2-c]pyridin]-4′(1′H)-one. To a solution of tert-butyl 4-[4-[4-(4-oxospiro[5,6-dihydro-1H-pyrrolo[3,2-c]pyridine-7,1′-cyclopropane]-2-yl)-2-pyridyl]phenyl]piperazine-1-carboxylate (110 mg, 220 umol) in DCM (10 mL) was added HCl/dioxane (4 M, 1 mL). The mixture was stirred at 25° C. for 0.5 hr. On completion, the reaction mixture was concentrated under reduced pressure to remove solvent and give the title compound (100 mg) as a yellow solid. LC-MS (ESI⁺) m/z 400.0 (M+H)⁺.

### 1-(1-(2,6-dioxopiperidin-3-yl)-3-methyl-2-oxo-2,3-dihydro-1H-benzo[d]imidazol-5-yl)piperidine-4-carbaldehyde (Intermediate W)

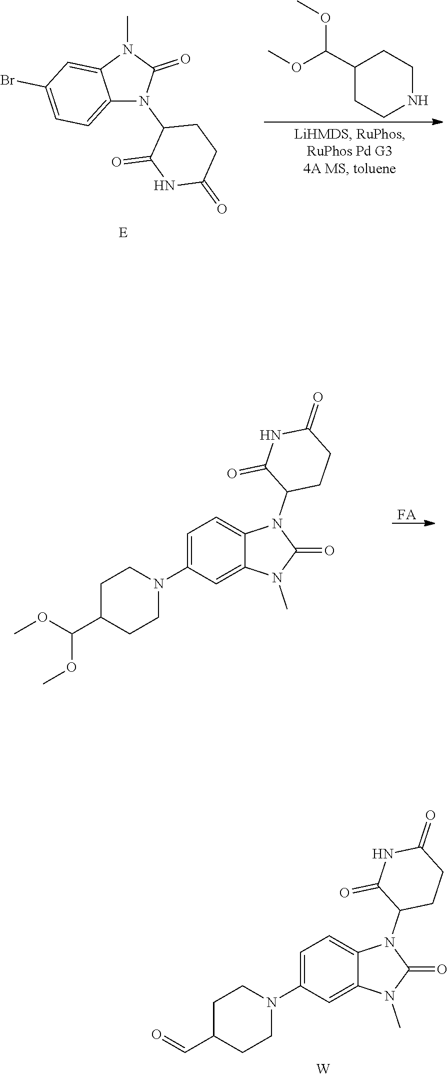

Step 1—3-(5-(4-(Dimethoxymethyl)piperidin-1-yl)-3-methyl-2-oxo-2,3-dihydro-1H-benzo[d]imidazol-1-yl)piperidine-2,6-dione. To a solution of 3-(5-bromo-3-methyl-2-oxo-benzimidazol-1-yl)piperidine-2,6-dione (5 g, 14.8 mmol, Intermediate E) in toluene (100 mL) was added 4-(dimethoxymethyl)piperidine (2.59 g, 16.3 mmol, CAS #188646-83-5), 4A MS (10 g) and LiHMDS (1 M, 73.9 mL). The mixture was purged with N₂ three times, then RuPhos Pd G3 (618 mg, 739 umol) and RuPhos (345 mg, 739 umol) was added and the mixture was purged with N₂ three times again. Then the mixture was stirred at 100° C. for 12 hrs under N₂ atmosphere. On completion, the mixture was quenched by adding FA until the pH=6, then filtered and the cake washed with THF (50 mL), then the filtrate was concentrated under reduced pressure to give a residue. The residue was purified by column chromatography (SiO₂, DCM/EtOAc=0/1 to 1/2, then added THF) to give the title compound (3 g, 49% yield) as a brown solid. LC-MS (ESI⁺) m/z 417.4 (M+H)⁺.

Step 2—1-(1-(2,6-dioxopiperidin-3-yl)-3-methyl-2-oxo-2,3-dihydro-1H-benzo[d]imidazol-5-yl)piperidine-4-carbaldehyde. To a solution of 3-[5-[4-(dimethoxymethyl)-1-piperidyl]-3-methyl-2-oxo-benzimidazol-1-yl]piperidine-2,6-dione (200 mg, 480 umol) was added formic acid (4.88 g, 106 mmol, 4 mL). The mixture was stirred at 25° C. for 0.5 hr. On completion, the reaction mixture was concentrated under reduced pressure to remove solvent to give the title compound (230 mg) as a brown oil. LC-MS (ESI⁺) m/z 389.1 (M+H)⁺.

### 2-(1-(2,6-dioxopiperidin-3-yl)-3-methyl-2-oxo-2,3-dihydro-1H-benzo[d]imidazol-4-yl)acetaldehyde (Intermediate X)

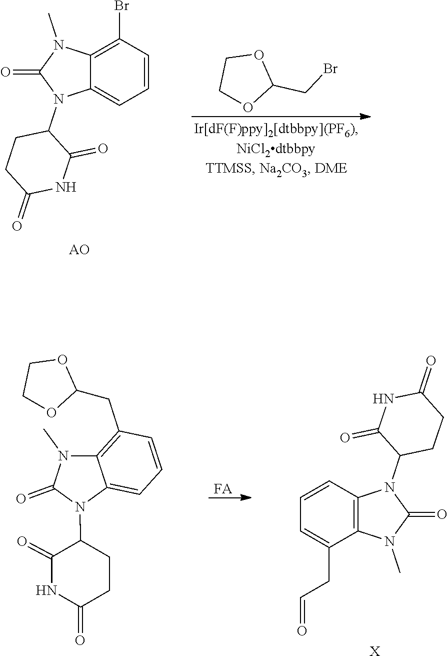

Step 1—3-(4-((1,3-Dioxolan-2-yl)methyl)-3-methyl-2-oxo-2,3-dihydro-1H-benzo[d]imidazol-1-yl)piperidine-2,6-dione. To an 15 mL vial equipped with a stir bar was added 3-(4-bromo-3-methyl-2-oxo-benzimidazol-1-yl)piperidine-2,6-dione (3 g, 8.87 mmol, Intermediate AO), 2-(bromomethyl)-1,3-dioxolane (1.93 g, 11.5 mmol CAS #4360-63-8), Ir[dF(F)ppy]₂[dtbbpy](PF6) (181 mg, 177 umol), NiCl₂.dtbbpy (70.6 mg, 177 umol), TTMSS (2.21 g, 8.87 mmol), and 2,6-Lutidine (1.90 g, 17.7 mmol) in DME (50 mL). The vial was sealed and placed under nitrogen. The reaction was stirred and irradiated with a 10 W blue LED lamp (3 cm away), with cooling water to keep the reaction temperature at 25° C. for 14 hrs. On completion, the reaction mixture was filtered and concentrated under reduced pressure to give a residue. The residue was purified by column chromatography (SiO₂, Petroleum ether/Ethyl acetate=10/1 to 0/1), DCM:EA=10:1) to give the title compound (2.8 g, 80% yield) as a yellow solid. LC-MS (ESI⁺) m/z 346.1 (M+H)⁺.

Step 2—2-(1-(2,6-Dioxopiperidin-3-yl)-3-methyl-2-oxo-2,3-dihydro-1H-benzo[d]imidazol-4-yl)acetaldehyde. 3-[4-(1,3-dioxolan-2-ylmethyl)-3-methyl-2-oxo-benzimidazol-1-yl]piperidine-2,6-dione (80 mg, 232 umol) was added into HCOOH (2 mL). The reaction was stirred at 25° C. for 1 hr. On completion, the reaction mixture was concentrated under reduced pressure to give the title compound (70 mg) as a brown solid. LC-MS (ESI⁺) m/z 301.7 (M+H)⁺.

### (R)-10,12-dimethyl-3-(4-(piperazin-1-yl)phenyl)-9,10,11,12-tetrahydro-8H-[1,4]diazepino[5′,6′:4,5]thieno[3,2-f]quinolin-8-one (Intermediate Y)

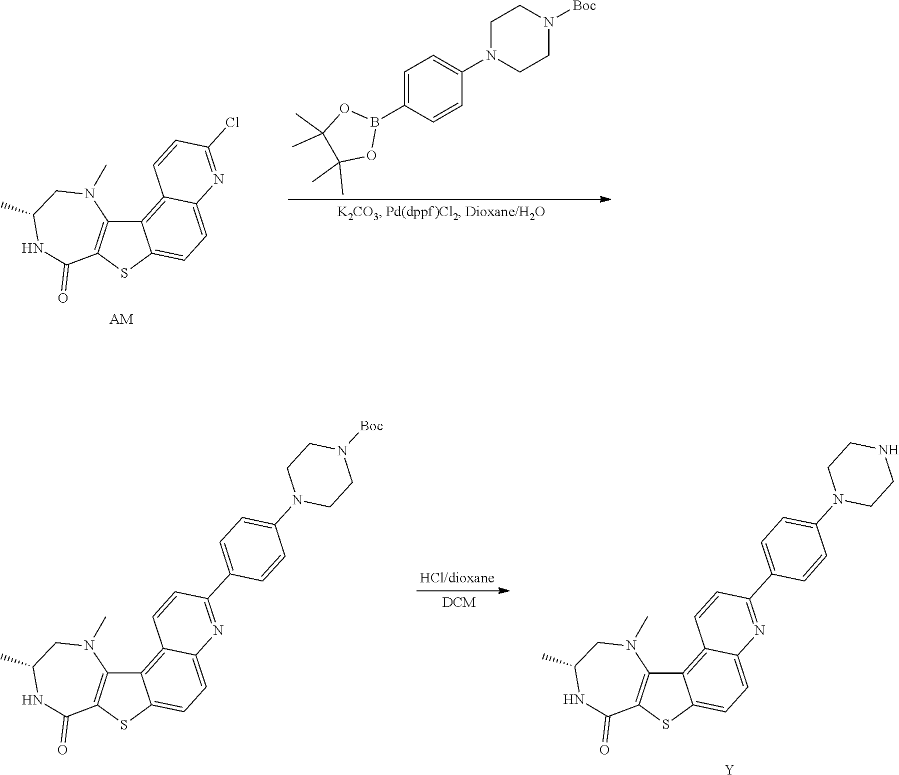

Step 1 (R)-tert-butyl 4-(4-(10,12-dimethyl-8-oxo-9,10,11,12-tetrahydro-8H-[1,4]diazepino[5′,6′:4,5]thieno[3,2-f]quinolin-3-yl)phenyl)piperazine-1-carboxylate. A mixture of (R)-3-chloro-10,12-dimethyl-9,10,11,12-tetrahydro-8H-[1,4]diazepino[5′,6′:4,5]thieno[3,2-f]quinolin-8-one (500 mg, 1.51 mmol, Intermediate AM), tert-butyl 4-(4-(4,4,5,5-tetramethyl-1,3,2-dioxaborolan-2-yl)phenyl)piperazine-1-carboxylate (585 mg, 1.51 mmol), Pd(dppf)Cl₂ (110 mg, 150 umol), and K₂CO₃ (624 mg, 4.52 mmol) in dioxane (10 mL) and H₂O (5 mL) was degassed and purged with N₂ three times. Then the mixture was stirred at 80° C. for 2 hrs under N₂ atmosphere. On completion, the crude product was triturated with H₂O (15 mL) at 25° C. for 5 min and then filtered to give the title compound (800 mg) as yellow solid. LC-MS (ESI⁺) m/z 558.2 (M+H)⁺.

Step 2—(R)-10,12-dimethyl-3-(4-(piperazin-1-yl)phenyl)-9,10,11,12-tetrahydro-8H-[1,4]diazepino[5′,6′:4,5]thieno[3,2-f]quinolin-8-one. To a solution of (R)-tert-butyl 4-(4-(10,12-dimethyl-8-oxo-9,10,11,12-tetrahydro-8H-[1,4]diazepino[5′,6′:4,5]thieno[3,2-f]quinolin-3-yl)phenyl)piperazine-1-carboxylate (800 mg, 1.43 mmol) in DCM (10 mL) was added HCl/dioxane (4 M, 358 uL). The mixture was stirred at 25° C. for 1 hr. On completion, the reaction mixture was concentrated under reduced pressure to give the title compound (700 mg, HCl) as yellow solid. LC-MS (ESI⁺) m/z 458.3 (M+H)⁺.

### 7-Azaspiro[4.5]decane-8,10-dione (Intermediate Z)

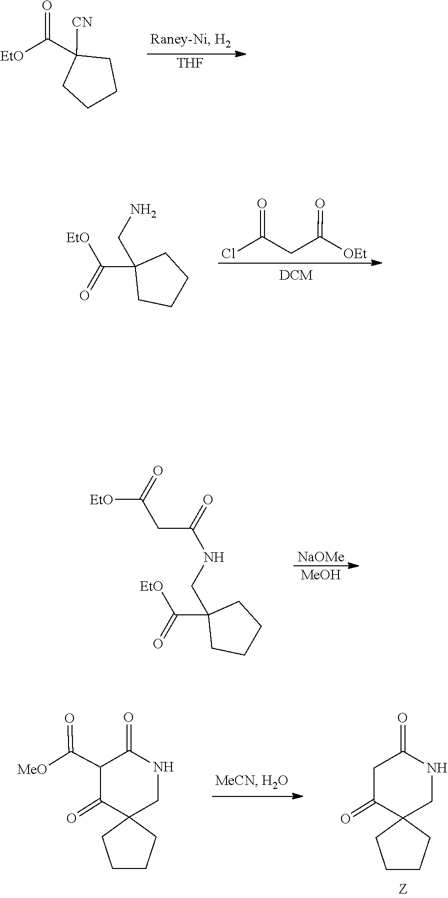

Step 1—Ethyl 1-(aminomethyl)cyclopentanecarboxylate. To a solution of methyl 1-cyanocyclopentanecarboxylate (3 g, 18 mmol, CAS #28247-14-5) in THF (100 mL) was added Raney-Ni (1.54 g, 17.9 mmol) under N₂ atmosphere. The suspension was degassed and purged with H₂ three times. The mixture was then stirred under H₂ (40 Psi) at 25° C. for 4 hrs. On completion, the reaction mixture was concentrated under reduced pressure to give the title compound (3 g) as a light yellow liquid. LC-MS (ESI⁺) m/z 172.2. (M+H)⁺.

Step 2—Ethyl 1-((3-ethoxy-3-oxopropanamido)methyl)cyclopentanecarboxylate. To a solution of ethyl 1-(aminomethyl)cyclopentanecarboxylate (3 g, 17.5 mmol) in DCM (40 mL) was added ethyl 3-chloro-3-oxo-propanoate (3.17 g, 21.0 mmol, 2.64 mL, CAS #36239-09-5). The reaction was stirred at 0° C. for 2 hrs. On completion, the reaction mixture was quenched with H₂O (20 mL) at 25° C., and extracted with DCM (50 mL×3). The combined organic layers were dried over Na₂SO₄, filtered and concentrated under reduced pressure to give a residue. The residue was purified by column chromatography (SiO₂, Petroleum ether/Ethyl acetate=10/1 to 3/1) to give a title compound (3.7 g, 70% yield) as a colorless liquid. LC-MS (ESI⁺) m/z 286.1 (M+H)⁺.

Step 3—Methyl 8,10-dioxo-7-azaspiro[4.5]decane-9-carboxylate. To a solution of ethyl 1-[[(3-ethoxy-3-oxo-propanoyl)amino]methyl]cyclopentanecarboxylate (3.5 g, 12 mmol) in MeOH (15 mL) was added NaOMe (2.43 g, 13.5 mmol, 5 mL, 30% solution). The reaction was stirred at 60° C. for 6 hrs. On completion, the reaction mixture was quenched with 1M HCl until the pH=5 at 25° C., and then extracted with EA 150 (50 mL×3). The combined organic layers were washed with NaCl (25 mL×2), dried over Na₂SO₄, filtered and concentrated under reduced pressure to give the title compound (2 g) as a yellow oil. LC-MS (ESI⁺) m/z 226.1 (M+H)⁺.

Step 4—7-Azaspiro[4.5]decane-8,10-dione. A solution of methyl 8,10-dioxo-7-azaspiro[4.5]decane-9-carboxylate (1.8 g, 8.0 mmol) in MeCN (20 mL) and H₂O (5 mL) was stirred at 80° C. for 4 hrs. On completion, the reaction mixture was quenched with H₂O (20 mL) at 25° C., and then extracted with EA (50 mL×3). The combined organic layers were washed with sat. NaCl (50 mL×3), dried over Na₂SO₄, filtered and concentrated under reduced pressure to give a residue. The residue was purified by column chromatography (SiO₂, Petroleum ether/Ethyl acetate=10/1 to 0/1) to give a title compound (270 mg, 20% yield) as a brown solid. LC-MS (ESI⁺) m/z 168.2 (M+H)⁺.

### 2′-(2-chloropyridin-4-yl)-5′,6′-dihydrospiro[cyclopentane-1,7′-pyrrolo[3,2-c]pyridin]-4′(1′H),-one (Intermediate AA)

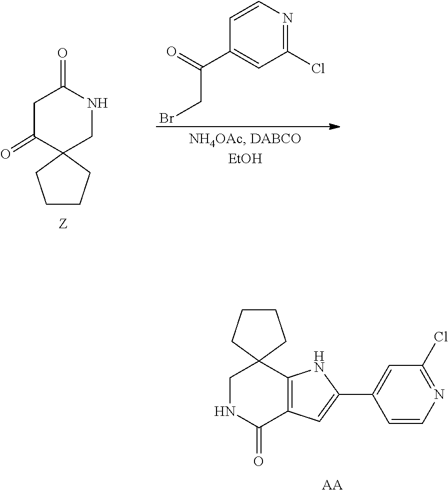 [SMILES: O=C1CC(=O)C2(CCCC2)CN1 ; O=C(CBr)c1ccnc(Cl)c1 ; O=C1NCC2(CCCC2)c2[nH]c(-c3ccnc(Cl)c3)cc21 ; "AA"]

To a solution of 7-azaspiro[4.5]decane-8,10-dione (170 mg, 1.02 mmol, Intermediate Z) in EtOH (2 mL) was added 2-bromo-1-(2-chloro-4-pyridyl)ethanone (358 mg, 1.53 mmol), NH₄OAc (1.18 g, 15.3 mmol) and DABCO (114 mg, 1.02 mmol). The mixture was stirred at 50° C. for 1 hr. On completion, the reaction mixture was quenched with H₂O (10 mL) at 25° C., and then extracted with EA (15 mL×3). The combined organic layers were washed with sat. NaCl (10 mL×2), dried over Na₂SO₄, filtered and concentrated under reduced pressure to give a residue. The residue was purified by column chromatography (SiO₂, Petroleum ether/Ethyl acetate=1/1 to DCM:MeOH=20/1) to give a title compound (280 mg, 88% yield) as a yellow solid. LC-MS (ESI⁺) m/z 301.9 (M+H)⁺.

### 2′-(2-(4-(piperazin-1-yl)phenyl)pyridin-4-yl)-5′,6′-dihydrospiro[cyclopentane-1,7′-pyrrolo[3,2-c]pyridin]-4′(1′H)-one (Intermediate AB)

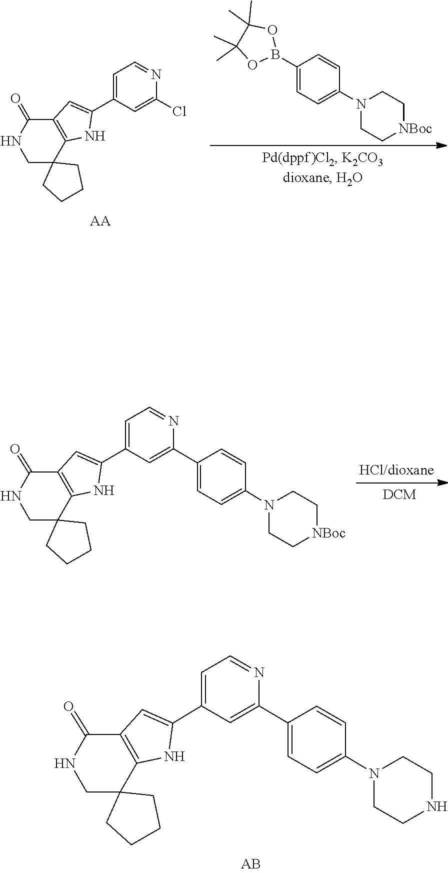

Step 1—Tert-butyl 4-(4-(4-(4′-oxo-1′,4′,5′,6′-tetrahydrospiro[cyclopentane-1,7′-pyrrolo[3,2-c]pyridin]-2′-yl)pyridin-2-yl)phenyl)piperazine-1-carboxylate. To a solution of 2-(2-chloro-4-pyridyl)spiro[5,6-dihydro-1H-pyrrolo[3,2-c]pyridine-7,1′-cyclopentane]-4-one (600 mg, 1.99 mmol, Intermediate AA) in dioxane (10 mL) and H₂O (2.5 mL) was added tert-butyl 4-[4-(4,4,5,5-tetramethyl-1,3,2-dioxaborolan-2-yl)phenyl]piperazine-1-carboxylate (1.16 g, 2.98 mmol, CAS #470478-90-1), K₂CO₃ (824 mg, 5.96 mmol) and Pd(dppf)Cl₂ (146 mg, 199 umol). The reaction was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=0/1 to DCM:MeOH=20:1) to give a title compound (180 mg, 12% yield) as a brown solid. LC-MS (ESI⁺) m/z 528.4 (M+H)⁺.

Step 2—2′-(2-(4-(Piperazin-1-yl)phenyl)pyridin-4-yl)-5′,6′-dihydrospiro[cyclopentane-1,7′-pyrrolo[3,2-c]pyridin]-4′(1′H)-one. To a solution of tert-butyl 4-[4-[4-(4-oxospiro[5,6-dihydro-1H-pyrrolo[3,2-c]pyridine-7,1′-cyclopentane]-2-yl)-2-pyridyl]phenyl]piperazine-1-carboxylate (180 mg, 341 umol) in DCM (8 mL) was added HCl/dioxane (4 M, 2 mL). The reaction was stirred at 25° C. for 1 hr. On completion, the reaction mixture was concentrated under reduced pressure to give the title compound (200 mg) as a yellow solid. LC-MS (ESI⁺) m/z 427.9 (M+H)⁺.

### 2-(2-chloropyridin-4-yl)-6,7-dihydro-1H-pyrrolo[3,2-c]pyridin-4(5H)-one (Intermediate AC)

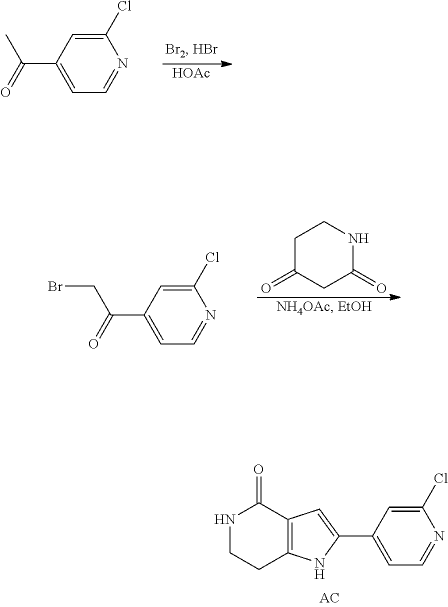

Step 1—2-Bromo-1-(2-chloropyridin-4-yl)ethanone. To a solution of 1-(2-chloropyridin-4-yl)ethanone (3 g, 19.3 mmol, CAS #23794-15-2) in HOAc (80 mL) was added HBr (4.73 g, 19.3 mmol, 3.17 mL) at 25° C. After addition, the mixture was stirred rt for 5 min, and then Br₂ (3.39 g, 21.2 mmol, 1.09 mL) was added dropwise at 25° C. The resulting mixture was stirred at 25° C. for 2 hours. On completion, the reaction mixture was filtered and concentrated under reduced pressure to give the title compound (5.2 g). LC-MS (ESI⁺) m/z 235.9 (M+H)⁺.

Step 2—2-(2-chloropyridin-4-yl)-6,7-dihydro-1H-pyrrolo[3,2-c]pyridin-4(5H)-one. A mixture of 2-bromo-1-(2-chloropyridin-4-yl)ethanone (4.7 g, 20.0 mmol), piperidine-2,4-dione (2.27 g, 20.0 mmol, CAS #50607-30-2), and NH₄OAc (6.18 g, 80.2 mmol) in EtOH (50 mL) was degassed and purged with N₂ three times. Then the mixture was stirred at 25° C. for 2 hrs under N₂ atmosphere. On completion, the reaction mixture was triturated with PE. The filter cake washed with PE and dried to give the title compound (2.6 g, 52% yield) as a white solid. LC-MS (ESI⁺) m/z 248.0 (M+H)⁺.

### 2-(2-(4-(piperazin-1-yl)phenyl)pyridin-4-yl)-6,7-dihydro-1H-pyrrolo[3,2-c]pyridin-4(5H)-one (Intermediate AD)

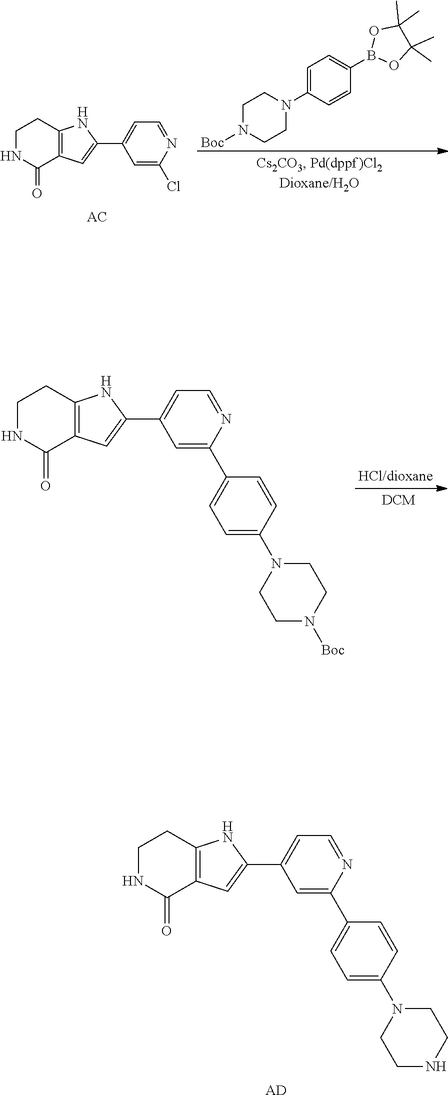 [SMILES: O=C1NCCc2[nH]c(-c3ccnc(Cl)c3)cc21 ; CC(C)(C)OC(=O)N1CCN(c2ccc(B3OC(C)(C)C(C)(C)O3)cc2)CC1 ; CC(C)(C)OC(=O)N1CCN(c2ccc(-c3cc(-c4cc5c([nH]4)CCNC5=O)ccn3)cc2)CC1 ; O=C1NCCc2[nH]c(-c3ccnc(-c4ccc(N5CCNCC5)cc4)c3)cc21 ; "AD"]

Step 1—Tert-butyl 4-(4-(4-(4-oxo-4,5,6,7-tetrahydro-1H-pyrrolo[3,2-c]pyridin-2-yl)pyridin-2-yl)phenyl)piperazine-1-carboxylate. To a solution of tert-butyl 4-[4-(4,4,5,5-tetramethyl-1,3,2-dioxaborolan-2-yl)phenyl]piperazine-1-carboxylate (941 mg, 2.42 mmol, CAS #470478-90-1) and 2-(2-chloro-4-pyridyl)-1,5,6,7-tetrahydropyrrolo[3,2-c]pyridin-4-one (500 mg, 2.02 mmol, Intermediate AC) in dioxane (10 mL) and H₂O (2 mL) was added Pd(dppf)Cl₂ (118 mg, 162 umol) and Cs₂CO₃ (1.97 g, 6.06 mmol) at 25° C., then the mixture was stirred at 100° C. for 2 hrs. On completion, the reaction mixture was quenched with water (20 mL) and extracted by ethyl acetate/dichloromethane (3×30 mL). The extracts were washed by brine (60 mL) and dried over anhydrous sodium sulfate, filtered and concentrated in vacuo to get the crude residue. The residue was purified by column chromatography (SiO₂, DCM:MeOH=15:1 to 10:1) to give the title compound (900 mg, 93% yield) as yellow solid. LC-MS (ESI⁺) m/z 474.1. (M+H)⁺.

Step 2—2-(2-(4-(Piperazin-1-yl)phenyl)pyridin-4-yl)-6,7-dihydro-1H-pyrrolo[3,2-c]pyridin-4(5H)-one. To a solution of tert-butyl 4-(4-(4-(4-oxo-4,5,6,7-tetrahydro-1H-pyrrolo[3,2-c]pyridin-2-yl)pyridin-2-yl)phenyl)piperazine-1-carboxylate (200 mg, 422 umol) in DCM (2 mL) was added HCl/dioxane (4 M, 0.5 mL) at 25° C., the mixture was stirred at 25° C. for 1 hr. On completion, the reaction mixture was filtered and concentrated in vacuo to give the title compound (200 mg) as white solid. LC-MS (ESI⁺) m/z 374.0. (M+H)⁺.

### 1-(4-(2,6-dioxopiperidin-3-yl)phenyl)piperidine-4-carbaldehyde (Intermediate AE)

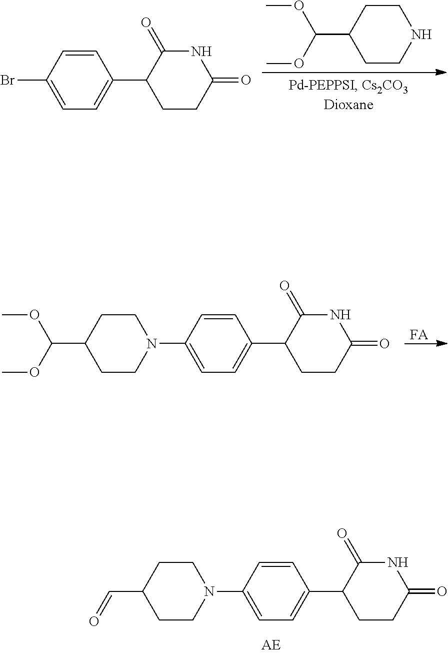

Step 1—3-(4-(4-(Dimethoxymethyl)piperidin-1-yl)phenyl)piperidine-2,6-dione. A mixture of 3-(4-bromophenyl)piperidine-2,6-dione (200 mg, 745 umol, CAS #1267337-47-2), 4-(dimethoxymethyl)piperidine (190 mg, 1.19 mmol, CAS #2152673-13-5), Cs₂CO₃ (729 mg, 2.24 mmol), 1,3-bis[2,6-bis(1-propylbutyl)phenyl]-4,5-dichloro-2H-imidazol-1-ium-2-ide;3-chloropyridine; dichloropalladium (72.5 mg, 74.6 umol) in dioxane (2 mL) was degassed and purged with N₂ three times. Then the mixture was stirred at 100° C. for 2 hrs under N₂ atmosphere. On completion, the reaction mixture was concentrated under reduced pressure and purified by column chromatography (SiO₂, Petroleum ether/Ethyl acetate=0/1) to give the title compound (100 mg, 39% yield) as a white solid. LC-MS (ESI⁺) m/z 347.1 (M+H)⁺.

Step 2—1-(4-(2,6-Dioxopiperidin-3-yl)phenyl)piperidine-4-carbaldehyde. 3-[4-[4-(Dimethoxymethyl)-1-piperidyl]phenyl]piperidine-2,6-dione (70 mg, 202 umol) was added to formic acid (1 mL). The mixture was stirred at 25° C. for 4 hrs. On completion, the reaction mixture was concentrated under reduced pressure to give the title compound (60 mg, 86% yield, FA) as yellow oil. LC-MS (ESI⁺) m/z 301.1 (M+H)⁺.

### 1-[1-(2,6-dioxo-3-piperidyl)-3-methyl-2-oxo-benzimidazol-4-yl]piperidine-4-carbaldehyde (Intermediate AF)

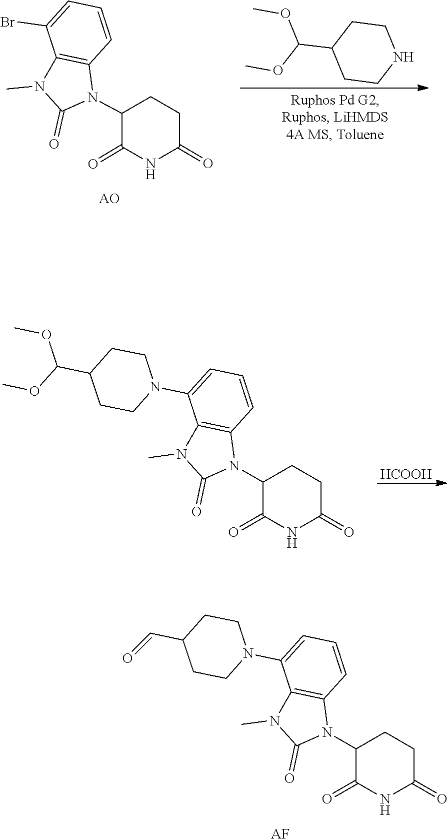 [SMILES: Cn1c(=O)n(C2CCC(=O)NC2=O)c2cccc(Br)c21 ; COC(OC)C1CCNCC1 ; COC(OC)C1CCN(c2cccc3c2n(C)c(=O)n3C2CCC(=O)NC2=O)CC1 ; "HCOOH" ; Cn1c(=O)n(C2CCC(=O)NC2=O)c2cccc(N3CCC(C=O)CC3)c21 ; "AF"]

Step 1—3-(4-(4-(Dimethoxymethyl)piperidin-1-yl)-3-methyl-2-oxo-2,3-dihydro-1H-benzo[d]imidazol-1-yl)piperidine-2,6-dione. To a solution of 3-(4-bromo-3-methyl-2-oxo-2,3-dihydro-1H-benzo[d]imidazol-1-yl)piperidine-2,6-dione (5 g, 14.8 mmol, Intermediate AO), 4-(dimethoxymethyl)piperidine (3.53 g, 22.1 mmol, CAS #188646-83-5) and 4 Å molecular sieves (10 g) in toluene (50 mL) was added LiHMDS (1 M, 73.9 mL) at 0° C., then RuPhos (345 mg, 739 umol) and [2-(2-aminophenyl)phenyl]-chloro-palladium;dicyclohexyl-[2-(2,6-diisopropoxyphenyl)phenyl]phosphane (574 mg, 739 umol) was added. The reaction was purged with N₂ three times, and then the mixture was stirred at 100° C. for 1 hr under N₂ atmosphere. On completion, the mixture was quenched by adding FA until pH=6 at 0° C., then the mixture was filtered and the cake was washed with THF (50 mL). The combined organic phase was concentrated under reduced pressure to give the crude product. The crude product was purified by reversed-phase HPLC (0.1% FA condition) to give the title compound (2.2 g, 32% yield, FA) as a white solid. LC-MS (ESI⁺) m/z 417.1 (M+H)⁺.

Step 2—1-(1-(2,6-dioxopiperidin-3-yl)-3-methyl-2-oxo-2,3-dihydro-1H-benzo[d]imidazol-4-yl)piperidine-4-carbaldehyde. To a solution of 3-(4-(4-(dimethoxymethyl)piperidin-1-yl)-3-methyl-2-oxo-2,3-dihydro-1H-benzo[d]imidazol-1-yl)piperidine-2,6-dione (80 mg, 192 umol) in HCOOH (2 mL) was stirred at 30° C. for 1 hr. On completion, the mixture was concentrated under reduced pressure to give the title compound (80 mg, FA) as a brown oil. LC-MS (ESI⁺) m/z 371.0 (M+H)⁺.

### (R)-tert-butyl 2-(2-chloropyridin-4-yl)-6-methyl-4-oxo-6,7-dihydrofuro[3,2-c]pyridine-5(4H)-carboxylate (Intermediate AG)

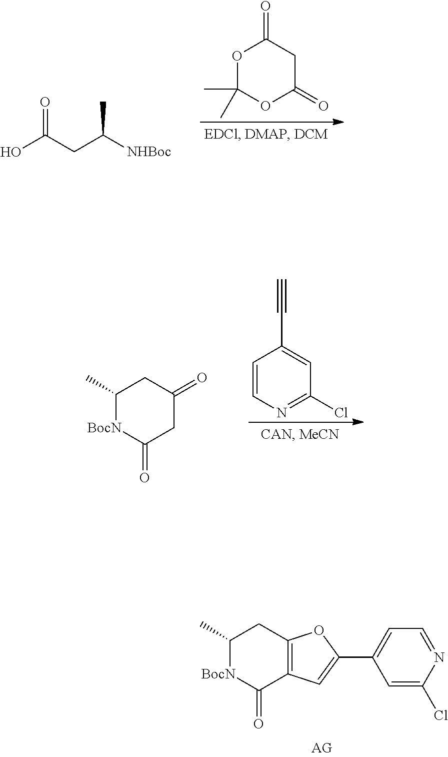

Step 1—(R)-tert-butyl 2-methyl-4,6-dioxopiperidine-1-carboxylate. To a solution of (3R)-3-(tert-butoxycarbonylamino)butanoic acid (5 g, 24.6 mmol, CAS #15991-23-8) and 2,2-dimethyl-1,3-dioxane-4,6-dione (3.55 g, 24.6 mmol, CAS #2033-24-1) in DCM (50 mL) was added EDCI (7.07 g, 36.90 mmol) and DMAP (4.51 g, 36.9 mmol). The mixture was stirred at 25° C. for 3 hrs, and then the reaction mixture was diluted with 1 M KHSO₄ (50 mL) and extracted with EA (70 mL×3). The combined organic layers were dried over Na₂SO₄, filtered and concentrated under reduced pressure to give a residue. The residue was added to EA (30 mL), and stirred at 80° C. for 5 hrs. Then the mixture was concentrated under reduced pressure to give the residue. The residue was purified by column chromatography (SiO₂, Petroleum ether/Ethyl acetate=1/0 to 2/1) to give the title compound (3.7 g, 60% yield) as a white solid. LC-MS (ESI⁺) m/z 172.1 (M-55)^{•}. ¹H NMR (400 MHz, DMSO-d₆) δ=11.08 (s, 1H), 4.95 (d, J=2.0 Hz, 1H), 3.34 (s, 1H), 2.88 (ddd, J=1.6, 6.4, 17.2 Hz, 1H), 2.08 (d, J=16.8 Hz, 1H), 1.98 (s, 1H), 1.42 (s, 9H), 1.19 (d, J=6.4 Hz, 3H).

Step 2—(R)-tert-butyl 2-(2-chloropyridin-4-yl)-6-methyl-4-oxo-6,7-dihydrofuro[3,2-c]pyridine-5(4H)-carboxylate. To a solution of tert-butyl (2R)-2-methyl-4,6-dioxo-piperidine-1-carboxylate (1 g, 4.4 mmol) in ACN (40 mL) was added CAN (4.82 g, 8.8 mmol) and 2-chloro-4-ethynyl-pyridine (3.03 g, 22.0 mmol). The mixture was stirred at 0° C. for 3 hrs. On completion, the reaction mixture was diluted with H₂O (30 mL) and extracted with EA (50 mL×3). The combined organic layers were dried over Na₂SO₄, filtered and concentrated under reduced pressure to give a residue. The residue was purified by column chromatography (SiO₂, Petroleum ether/Ethyl acetate=1/0 to 2/1) to give the title compound (626 mg, 38% yield) as a yellow solid. LC-MS (ESI⁺) m/z 363.1 (M+H). ¹H NMR (400 MHz, DMSO-d₆) δ=8.43 (d, J=5.2 Hz, 1H), 7.73 (s, 1H), 7.70 (s, 1H), 7.62 (dd, J=1.6, 5.2 Hz, 1H), 4.88-4.76 (m, 1H), 3.09 (dd, J=6.0, 16.8 Hz, 1H), 2.35 (dd, J=1.6, 16.8 Hz, 1H), 1.60 (s, 9H), 1.20 (d, J=6.8 Hz, 3H).

### (R)-6-methyl-2-(6′-(piperazin-1-yl)-[2,3′-bipyridin]-4-yl)-6,7-dihydrofuro[3,2-c]pyridin-4(5H)-one (Intermediate AH)

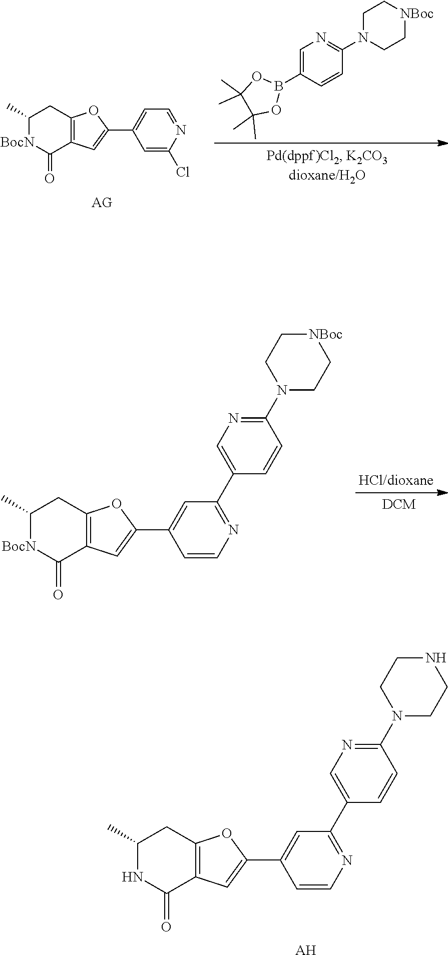 [SMILES: C[C@@H]1Cc2oc(-c3ccnc(Cl)c3)cc2C(=O)N1C(=O)OC(C)(C)C ; CC(C)(C)OC(=O)N1CCN(c2ccc(B3OC(C)(C)C(C)(C)O3)cn2)CC1 ; C[C@@H]1Cc2oc(-c3ccnc(-c4ccc(N5CCN(C(=O)OC(C)(C)C)CC5)nc4)c3)cc2C(=O)N1C(=O)OC(C)(C)C ; C[C@@H]1Cc2oc(-c3ccnc(-c4ccc(N5CCNCC5)nc4)c3)cc2C(=O)N1 ; "AH"]

Step 1—(R)-tert-butyl 2-(6′-(4-(tert-butoxycarbonyl)piperazin-1-yl)-[2,3′-bipyridin]-4-yl)-6-methyl-4-oxo-6,7-dihydrofuro[3,2-c]pyridine-5(4H)-carboxylate. A mixture of tert-butyl (6R)-2-(2-chloro-4-pyridyl)-6-methyl-4-oxo-6,7-dihydrofuro[3,2-c]pyridine-5-carboxylate (700 mg, 1.93 mmol, Intermediate AG), tert-butyl 4-[5-(4,4,5,5-tetramethyl-1,3,2- dioxaborolan-2-yl)-2-pyridyl]piperazine-1-carboxylate (1.13 g, 2.89 mmol, CAS #496786-98-2), K₂CO₃ (780 mg, 5.79 mmol), and Pd(dppf)Cl₂.CH₂Cl₂ (158 mg, 193 umol) in dioxane (8 mL) and H₂O (3 mL) was degassed and purged with N₂ three times. Then the mixture was stirred at 80° C. for 2 hrs under N₂ atmosphere. On completion, to the reaction mixture was added 3 g silica powder, and concentrated under reduced pressure to give a residue. The residue was purified by column chromatography (SiO₂, Petroleum ether/Ethyl acetate=1/0 to 1/2) to give the title compound (790 mg, 63% yield) as a yellow solid. LC-MS (ESI⁺) m/z 590.6 (M+H)⁺.

Step 2—(R)-6-methyl-2-(6′-(piperazin-1-yl)-[2,3′-bipyridin]-4-yl)-6,7-dihydrofuro[3,2-c]pyridin-4(5H)-one. To a solution of tert-butyl (6R)-2-[2-[6-(4-tert-butoxycarbonylpiperazin-1-yl)-3-pyridyl]-4-pyridyl]-6- methyl-4-oxo-6,7-dihydrofuro[3,2-c]pyridine-5-carboxylate (200 mg, 339 umol) in DCM (8 mL) was added HCl/dioxane (4 M, 2 mL). The mixture was stirred at 25° C. for 2 hrs. On completion, the reaction mixture was concentrated under reduced pressure to give the title compound as an orange solid (180 mg, HCl). LC-MS (ESI⁺) m/z 390.3 (M+H)⁺.

### (R)-3-(1,4-diazepan-1-yl)-10-methyl-9,10,11,12-tetrahydro-8H-[1,4]diazepino[5′,6′:4,5]thieno[3,2-f]quinolin-8-one (Intermediate AI)

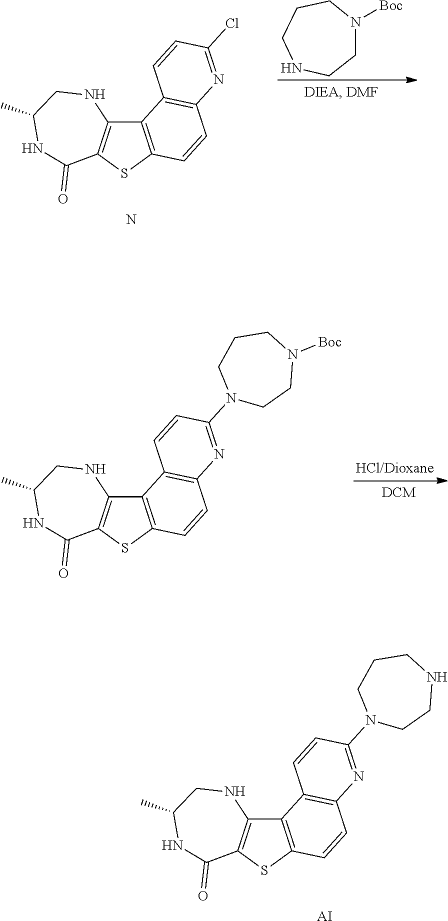

Step 1—Tert-butyl (R)-4-(10-methyl-8-oxo-9,10,11,12-tetrahydro-8H-[1,4]diazepino[5′,6′:4,5]thieno[3,2-f]quinolin-3-yl)-1,4-diazepane-1-carboxylate. To a solution of tert-butyl 1,4-diazepane-1-carboxylate (2.84 g, 14.2 mmol, CAS #112275-50-0) and (15R)-5-chloro-15-methyl-11-thia-6,14,17-triazatetracyclo[8.8.0.02,7.012,18]octadeca-1(10),2(7),3,5,8,12(18)-hexaen-13-one (3 g, 9.44 mmol, Intermediate N) in DMF (30 mL) was added DIEA (3.66 g, 28.3 mmol) at 25° C., then the mixture was stirred at 110° C. for 72 hrs. On completion, the reaction mixture was filtered and the filtrate was concentrated under vacuum. The residue was purified by reversed-phase HPLC (0.1% NH₃H₂O) to give the title compound (1.2 g, 26% yield) as a yellow solid. LC-MS (ESI⁺) m/z 482.2 (M+H)⁺.

Step 2—(R)-3-(1,4-diazepan-1-yl)-10-methyl-9,10,11,12-tetrahydro-8H-[1,4]diazepino[5′,6′:4,5]thieno[3,2-f]quinolin-8-one. To a solution of tert-butyl 4-[(15R)-15-methyl-13-oxo-11-thia-6,14,17-triazatetracyclo[8.8.0.02,7.012,18]octadeca-1(10),2(7),3,5,8,12(18)-hexaen-5-yl]-1,4-diazepane-1-carboxylate (400 mg, 830 umol) in DCM (8 mL) was added HCl/dioxane (4 M, 0.8 mL). The mixture was stirred at 25° C. for 4 hrs. On completion, the reaction mixture was concentrated under reduced pressure to give the title compound (300 mg) as yellow solid. LC-MS (ESI⁺) m/z 382.0 (M+H)⁺.

Step 1—4-(2-((Tert-butyldimethylsilyl)oxy)ethyl)piperidine (Intermediate AJ)

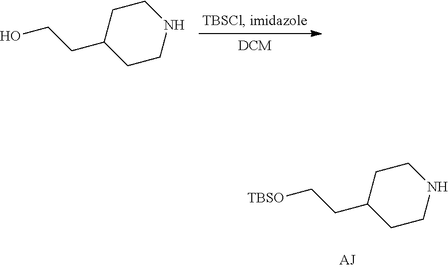 [SMILES: OCCC1CCNCC1 ; CC(C)(C)[Si](C)(C)OCCC1CCNCC1]

A mixture of 2-(4-piperidyl)ethanol (3 g, 23.2 mmol), TBSCl (4.72 g, 31.3 mmol), imidazole (1.74 g, 25.5 mmol) in DCM (30 mL) was degassed and purged with N₂ three times. Then the mixture was stirred at 25° C. for 12 hrs under N₂ atmosphere. On completion, the reaction mixture was quenched by addition of H₂O (60 mL) at 25° C., and then extracted with DCM (30 mL×2). The combined organic layers were washed with brine (30 mL×2), dried over Na₂SO₄, filtered and concentrated under reduced pressure to give a residue. The residue was purified by column chromatography (SiO₂, DCM:MeOH=1/0 to 10/1) to give the title compound (4.3 g, 76% yield) as a white solid. ¹H NMR (400 MHz, DMSO-d₆) δ =3.61 (t, J=6.5 Hz, 2H), 3.08 (br d, J=12.4 Hz, 2H), 2.65 (dt, J=2.3, 12.4 Hz, 2H), 1.68 (br d, J=12.9 Hz, 2H), 1.60-1.50 (m, 1H), 1.39 (q, J=6.5 Hz, 2H), 1.26-1.14 (m, 2H), 0.86 (s, 9H), 0.02 (s, 6H) LC-MS (ESI⁺) m/z 244.0 (M+H)⁺.

### 2-(1-(1-(2,6-dioxopiperidin-3-yl)-3-methyl-2-oxo-2,3-dihydro-1H-benzo[d]imidazol-5-yl)piperidin-4-yl)acetaldehyde (Intermediate AK)

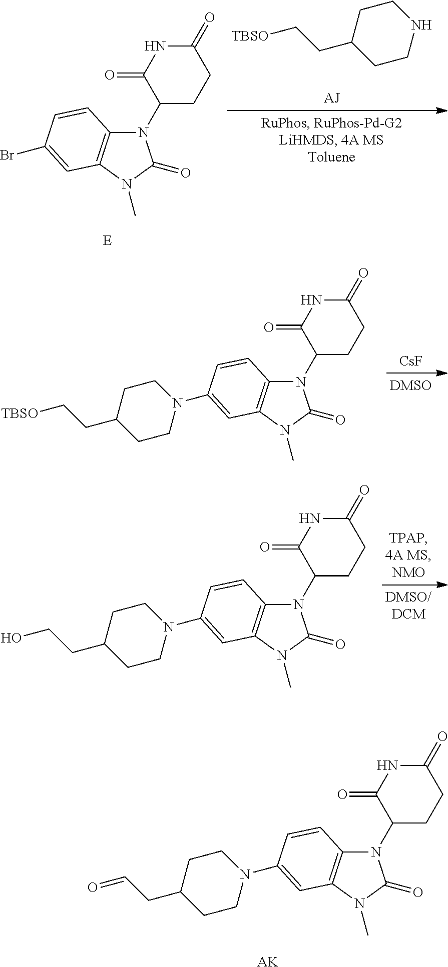

Step 1—3-(5-(4-(2-((Tert-butyldimethylsilyl)oxy)ethyl)piperidin-1-yl)-3-methyl-2-oxo-2,3-dihydro-1H-benzo[d]imidazol-1-yl)piperidine-2,6-dione. To a solution of 3-(5-bromo-3-methyl-2-oxo-benzimidazol-1-yl)piperidine-2,6-dione (3 g, 9 mmol, Intermediate E) in toluene (50 mL) was added tert-butyl-dimethyl-[2-(4-piperidyl)ethoxy]silane (3.89 g, 15.9 mmol, Intermediate AJ), 4 Å molecular sieves (4 g) and LiHMDS (1 M, 44.3 mL) and purged with N₂ three times. Then RuPhos (414 mg, 887 umol) and [2-(2-aminophenyl)phenyl]-chloro-palladium;dicyclohexyl-[2-(2,6-diisopropoxyphenyl) phenyl]phosphane (551 mg, 709 umol) was added, and the mixture was stirred at 100° C. for 1 hr under N₂ atmosphere. On completion, the mixture was quenched by adding FA until pH 6 at 0° C. The resulting mixture was filtered and the filter cake was washed with THF (200 mL), the combined organic layers were concentrated under reduced pressure to give the title compound (10 g) as a brown solid. LC-MS (ESI⁺) m/z 501.2 (M+H)⁺.

Step 2—3-(5-(4-(2-Hydroxyethyl)piperidin-1-yl)-3-methyl-2-oxo-2,3-dihydro-1H-benzo[d]imidazol-1-yl)piperidine-2,6-dione. To a solution of 3-[5-[4-[2-[tert-butyl(dimethyl)silyl]oxyethyl]-1-piperidyl]-3-methyl-2-oxo-benzimidazol-1-yl]piperidine-2,6-dione (3 g, 6 mmol) in DMSO (10 mL) was added CsF (4.55 g, 29.9 mmol). The mixture was stirred at 50° C. for 2 hrs. On completion, the reaction mixture was filtered and the filtrate was purified by prep-HPLC (FA condition) to give the title compound (400 mg, 17% yield) as a brown oil. LC-MS (ESI⁺) m/z 387.2 (M+H)⁺.

Step 3—2-(1-(1-(2,6-Dioxopiperidin-3-yl)-3-methyl-2-oxo-2,3-dihydro-1H-benzo[d]imidazol-5-yl)piperidin-4-yl)acetaldehyde. To a solution of 3-[5-[4-(2-hydroxyethyl)-1-piperidyl]-3-methyl-2-oxo-benzimidazol-1-yl]piperidine-2,6-dione (50 mg, 129 umol) in DMSO (1 mL) and DCM (1 mL) was added TPAP (18.1 mg, 51.7 umol), 4 Å molecular sieves (100 mg) and NMO (30.3 mg, 258 umol). The mixture was stirred at 0° C. for 0.5 hr. On completion, the reaction mixture was filtered and the filtrate was concentrated under reduced pressure to give the title compound (50 mg) as black liquid. LC-MS (ESI⁺) m/z 385.2 (M+H)⁺.

### (R)-10-methyl-3-(piperazin-1-yl)-9,10,11,12-tetrahydro-8H-[1,4]diazepino[5′,6′:4,5]thieno[3,2-f]quinolin-8-one (Intermediate AL)

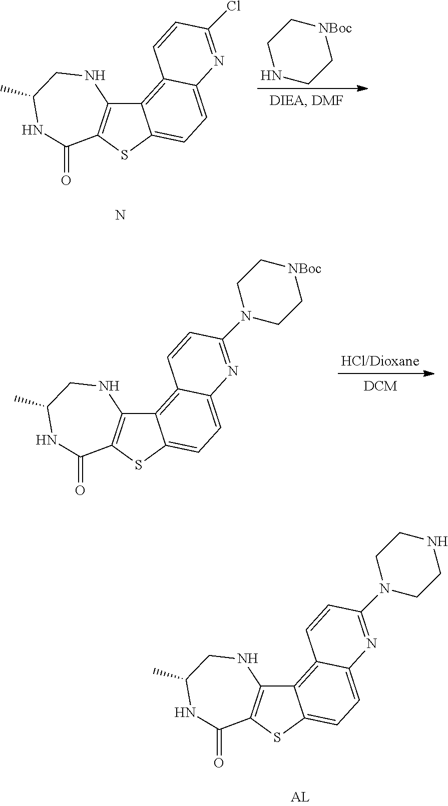

Step 1—(R)-tert-butyl 4-(10-methyl-8-oxo-9,10,11,12-tetrahydro-8H-[1,4]diazepino[5′,6′:4,5]thieno[3,2-f]quinolin-3-yl)piperazine-1-carboxylate. A mixture of (R)-3-chloro-10-methyl-9,10,11,12-tetrahydro-8H-[1,4]diazepino[5′,6′:4,5]thieno[3,2-f]quinolin-8-one (500 mg, 1.57 mmol, Intermediate N), tert-butyl piperazine-1-carboxylate (880 mg, 4.72 mmol), and DIEA (610 mg, 4.72 mmol, 822 uL) in DMF (5 mL) was degassed and purged with N₂ three times. Then the mixture was stirred at 100° C. for 12 hrs under N₂ atmosphere. On completion, the reaction mixture was quenched with H₂O (10 mL), and then extracted with EA (4 mL×3). The combined organic layers were washed with brine (5 mL×2), dried over Na₂SO₄, filtered and concentrated under reduced pressure to give the title compound (140 mg) as a white solid. LC-MS (ESI⁺) m/z 468.1 (M+H)⁺.

Step 2—(R)-10-methyl-3-(piperazin-1-yl)-9,10,11,12-tetrahydro-8H-[1,4]diazepino[5′,6′:4,5]thieno[3,2-f]quinolin-8-one. To a solution of (R)-tert-butyl 4-(10-methyl-8-oxo-9,10,11,12-tetrahydro-8H-[1,4]diazepino[5′,6′:4,5]thieno[3,2-f]quinolin-3-yl)piperazine-1-carboxylate (140 mg, 300 umol) in DCM (2 mL) was added HCl/Dioxane (4 M, 225 uL). The mixture was stirred at 25° C. for 2 hrs. On completion, the reaction mixture was concentrated under reduced pressure to give the title compound (100 mg, HCl) as a red solid. LC-MS (ESI⁺) m/z 368.1 (M+H)⁺.

### (R)-3-chloro-10,12-dimethyl-9,10,11,12-tetrahydro-8H-[1,4]diazepino[5′,6′:4,5]thieno[3,2-f]quinolin-8-one (Intermediate AM)

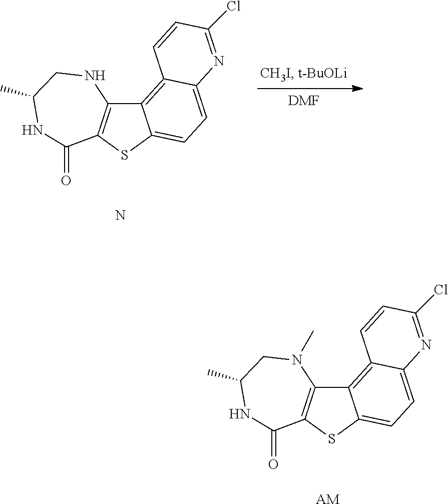

To a solution of (R)-3-chloro-10-methyl-9,10,11,12-tetrahydro-8H-[1,4]diazepino[5′,6′:4,5]thieno[3,2-f]quinolin-8-one (5 g, 15.7 mmol, Intermediate N) in DMF (50 mL) was added lithium 2-methylpropan-2-olate (2.2 M, 15.7 mL), then the reaction was stirred at 0° C. for 2 hrs. Next, iodomethane (1.79 g, 12.5 mmol, 783 uL) was added and the mixture was stirred at 0° C. for 2 hrs. On completion, the reaction mixture was concentrated under reduced pressure to give a residue. The residue was purified by column chromatography (SiO₂, Petroleum ether/Ethyl acetate=1/0 to 0/1) to give the compound (4.5 g, 46% yield) as a black solid. LC-MS (ESI⁺) m/z 331.9 (M+H)⁺.

### (R)-10,12-dimethyl-3-(6-(piperazin-1-yl)pyridin-3-yl)-9,10,11,12-tetrahydro-8H-[1,4]diazepino[5′,6′:4,5]thieno[3,2-f]quinolin-8-one (Intermediate AN)

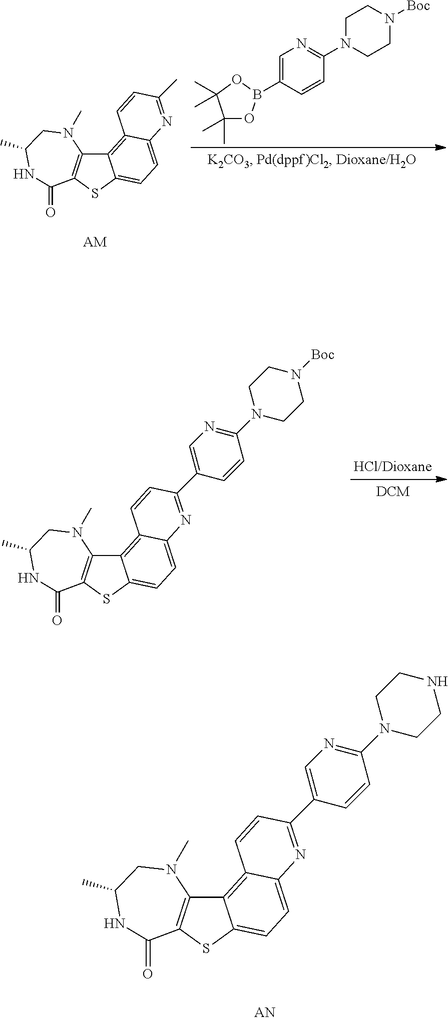

Step 1—(R)-tert-butyl 4-(5-(10,12-dimethyl-8-oxo-9,10,11,12-tetrahydro-8H-[1,4]diazepino[5′,6′:4,5]thieno[3,2-f]quinolin-3-yl)pyridin-2-yl)piperazine-1-carboxylate. A mixture of (R)-3-chloro-10,12-dimethyl-9,10,11,12-tetrahydro-8H-[1,4]diazepino[5′,6′:4,5]thieno[3,2-f]quinolin-8-one (1 g, 3.01 mmol, Intermediate AM), tert-butyl 4-(5-(4,4,5,5-tetramethyl-1,3,2-dioxaborolan-2-yl)pyridin-2-yl)piperazine-1-carboxylate (1.17 g, 3.01 mmol, CAS #496786-98-2), Pd(dppf)Cl₂ (220 mg, 301 umol), and K₂CO₃ (1.25 g, 9.04 mmol) in dioxane (10 mL) and H₂O (5 mL) was degassed and purged with N₂ three times. Then the mixture was stirred at 80° C. for 2 hrs under N₂ atmosphere. On completion, the crude product was triturated with H₂O, then filtered. The filter cake was dried under reduced pressure to give the title compound (1.5 g) as a yellow solid. LC-MS (ESI⁺) m/z 559.4 (M+H)⁺.

Step 2—(R)-10,12-dimethyl-3-(6-(piperazin-1-yl)pyridin-3-yl)-9,10,11,12-tetrahydro-8H-[1,4]diazepino[5′,6′:4,5]thieno[3,2-f]quinolin-8-one. To a solution of (R)-tert-butyl 4-(5-(10,12-dimethyl-8-oxo-9,10,11,12-tetrahydro-8H-[1,4]diazepino[5′,6′:4,5]thieno[3,2-f]quinolin-3-yl)pyridin-2-yl)piperazine-1-carboxylate (80 mg, 143 umol) in DCM (1 mL) was added HCl/dioxane (4 M, 35.8 uL). The mixture was stirred at 25° C. for 1 hr. On completion, the reaction mixture was concentrated under reduced pressure to give the compound (65 mg) as a red solid. LC-MS (ESI⁺) m/z 458.3 (M+H)⁺.

### 3-(4-Bromo-3-methyl-2-oxo-2,3-dihydro-1H-benzo[d]imidazol-1-yl)piperidine-2,6-dione (Intermediate AO)

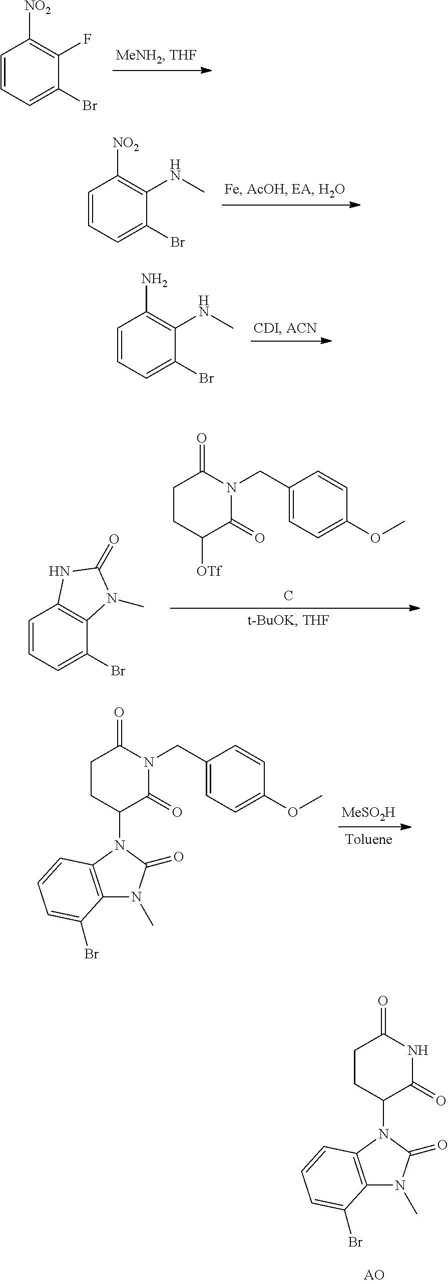

Step 1—2-Bromo-N-methyl-6-nitro-aniline. To a solution of 1-bromo-2-fluoro-3-nitro-benzene (40.0 g, 181 mmol, CAS #58534-94-4) in THF (40 mL) was added MeNH₂ (2 M, 400 mL). The reaction mixture was stirred at 60° C. for 12 hours. On completion, the reaction mixture was poured into sat.NaHCO₃ (30 mL) and extracted with EA (3×200 mL). The combined organic layers were washed with brine (2×200 mL), dried with anhydrous Na₂SO₄, filtered and concentrated in vacuo to give the title compound (40.0 g, 95% yield) as red oil. LC-MS (ESI⁺) m/z 230.9 (M+H)⁺.

Step 2—3-Bromo-N2-methyl-benzene-1,2-diamine. To a mixture of 2-bromo-N-methyl-6-nitro-aniline (23.0 g, 99.5 mmol) in EA (300 mL) and H₂O (10 mL) was added AcOH (100 mL). The mixture was warmed to 50° C. Then Fe (22.2 g, 398 mmol) was added to the reaction mixture and the mixture was heated to 80° C. about 4 hours. On completion, the reaction mixture was filtered and concentrated in vacuo. The residue was diluted with water (100 mL) and extracted with EA (3×200 mL). The combined organic layers was dried over Na₂SO₄, filtered and concentrated in vacuo to give the title compound (20.0 g, 99% yield) as red oil. ¹H NMR (400 MHz, DMSO-d₆) b 6.73-6.70 (m, 1H), 6.68-6.60 (m, 2H), 5.02 (s, 2H), 3.67 (s, 1H), 2.58 (s, 3H).

Step 3—4-Bromo-3-methyl-1H-benzimidazol-2-one. To a mixture of 3-bromo-N2-methyl-benzene-1,2-diamine (20.0 g, 99.4 mmol) in ACN (300 mL) was added CDI (32.2 g, 198 mmol). The reaction mixture was stirred at 85° C. for 12 hours under N₂ atmosphere. On completion, the reaction mixture was concentrated in vacuo. The reaction mixture was diluted with water (200 mL), where a solid precipitate was formed, which was filtered off. The solid was washed with water (1 L) and dried in vacuo to give the title compound (20.0 g, 88% yield) as white solid. ¹H NMR (400 MHz, DMSO-d₆) δ 11.17 (s, 1H), 7.14 (dd, J=1.2, 8.0 Hz, 1H), 7.00-6.95 (m, 1H), 6.93-6.87 (m, 1H), 3.55 (s, 3H).

Step 4—3-(4-Bromo-3-methyl-2-oxo-benzimidazol-1-yl)-1-[(4-methoxyphenyl)methyl]piperidine-2,6-dione. To a solution of 4-bromo-3-methyl-1H-benzimidazol-2-one (12.0 g, 52.8 mmol) in THF (300 mL) was added t-BuOK (7.12 g, 63.4 mmol). The reaction mixture was stirred at 0° C. for 0.5 hr. Subsequently, [1-[(4-methoxyphenyl)methyl]-2,6-dioxo-3-piperidyl]trifluoromethanesulfonate (20.1 g, 52.8 mmol, Intermediate C) in a solution of THF (100 mL) was added dropwise. The resulting reaction mixture was stirred at 20° C. for 0.5 hr under N₂. On completion, the reaction mixture was quenched with saturated NH₄Cl (100 mL), and extracted with ethyl acetate (200 mL). The combined organic layers were washed with brine (2×100 mL), dried over anhydrous sodium sulfate, filtered, the filtrate was concentrated in vacuo. The crude product was purified by reversed-phase HPLC (0.1% FA condition) to give the title compound (13.3 g, 55% yield) as a yellow solid. ¹H NMR (400 MHz, CDCl₃) b 7.38 (d, J=8.8 Hz, 2H), 7.22 (d, J=8.0 Hz, 1H), 6.84 (d, J=8.8 Hz, 2H), 6.80 (t, J=8.0 Hz, 1H), 6.48-6.40 (d, J=8.0 Hz, 1H), 5.22 (dd, J=5.2, 12.8 Hz, 1H), 5.04-4.93 (m, 2H), 3.81 (s, 3H), 3.80 (s, 3H), 3.12-2.98 (m, 1H), 2.93-2.77 (m, 1H), 2.62 (dq, J=4.4, 13.2 Hz, 1H), 2.20-2.17 (m, 1H).

Step 5—3-(4-bromo-3-methyl-2-oxo-2,3-dihydro-1H-benzo[d]imidazol-1-yl)piperidine-2,6-dione. A mixture of 3-(4-bromo-3-methyl-2-oxo-benzimidazol-1-yl)-1-[(4-methoxyphenyl)methyl]piperidine-2,6-dione (13.3 g, 29.0 mmol) in a mixed solvent of Tol. (80 mL) and methane sulfonic acid (40 mL) was degassed and purged with N₂ for 3 times, and then the mixture was stirred at 120° C. for 2 hrs under N₂ atmosphere. On completion, the reaction mixture was concentrated in vacuo to remove toluene. The residue was added 200 mL of ice water, and then white solid precipitate formed. The mixture was filtered and the filtered cake was collected and dried over in vacuo to give the title compound (7.30 g, 74% yield) as white solid. ¹H NMR (400 MHz, DMSO-d₆) b 11.13 (s, 1H), 7.25 (d, J=8.0 Hz, 1H), 7.17 (d, J=8.0 Hz, 1H), 7.05-6.93 (m, 1H), 5.41 (dd, J=5.2, 12.8 Hz, 1H), 3.64 (s, 3H), 2.96-2.83 (m, 1H), 2.78-2.59 (m, 2H), 2.08-2.00 (m, 1H).

### 3-(3-methyl-2-oxo-4-(3-oxoazetidin-1-yl)-2,3-dihydro-1H-benzo[d]imidazol-1-yl)piperidine-2,6-dione (Intermediate AP)

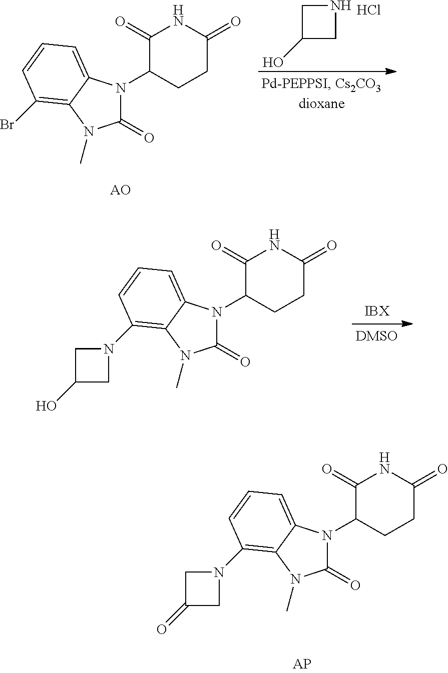 [SMILES: Cn1c(=O)n(C2CCC(=O)NC2=O)c2cccc(Br)c21 ; OC1CNC1 ; Cl ; Cn1c(=O)n(C2CCC(=O)NC2=O)c2cccc(N3CC(O)C3)c21 ; Cn1c(=O)n(C2CCC(=O)NC2=O)c2cccc(N3CC(=O)C3)c21]

Step 1—3-(4-(3-Hydroxyazetidin-1-yl)-3-methyl-2-oxo-2,3-dihydro-1H-benzo[d]imidazol-1-yl)piperidine-2,6-dione. To a solution of 3-(4-bromo-3-methyl-2-oxo-2,3-dihydro-1H-benzo[d]imidazol-1-yl)piperidine-2,6-dione (1 g, 2.96 mmol, Intermediate AO) in dioxane (10 mL) was added azetidin-3-ol hydrochloride (648 mg, 5.91 mmol, HCl, CAS #45347-82-8), 1,3-bis[2,6-bis(1-propylbutyl)phenyl]-4,5-dichloro-2H-imidazol-1-ium-2-ide;3-chloropyridine dichloropalladium (288 mg, 296 umol) and Cs₂CO₃ (2.41 g, 7.39 mmol) and the mixture was purged with N₂ three times. Then the mixture was stirred at 100° C. under microwave for 3 hrs under N₂ atmosphere. On completion, the mixture was concentrated under reduced pressure to give a residue. The residue was purified by column chromatography (SiO₂, DCM/EtOAc=0/1 to 1/2) to give the title compound (300 mg, 30% yield) as a brown solid. LC-MS (ESI⁺) m/z 331.1 (M+H)⁺.

Step 2—3-(3-Methyl-2-oxo-4-(3-oxoazetidin-1-yl)-2,3-dihydro-1H-benzo[d]imidazol-1-yl)piperidine-2,6-dione. To a solution of 3-(4-(3-hydroxyazetidin-1-yl)-3-methyl-2-oxo-2,3-dihydro-1H-benzo[d]imidazol-1-yl)piperidine-2,6-dione (250 mg, 757 umol) in DMSO (6 mL) was added IBX (424 mg, 1.51 mmol). The mixture was stirred at 20° C. for 12 hrs. On completion, the mixture was quenched with H₂O (20 mL), extracted with EtOAc (20 mL×3), washed with brine (15 mL×3) and dried over anhydrous Na₂SO₄. The mixture was then filtered and concentrated under reduced pressure to give the title compound (360 mg) as brown gum. LC-MS (ESI⁺) m/z 329.1 (M+H)⁺.

### (R)-10-methyl-3-(4-(piperazin-1-yl)phenyl)-9,10,11,12-tetrahydro-8H-[1,4]diazepino[5′,6′:4,5]thieno[3,2-f]quinolin-8-one (Intermediate AQ)

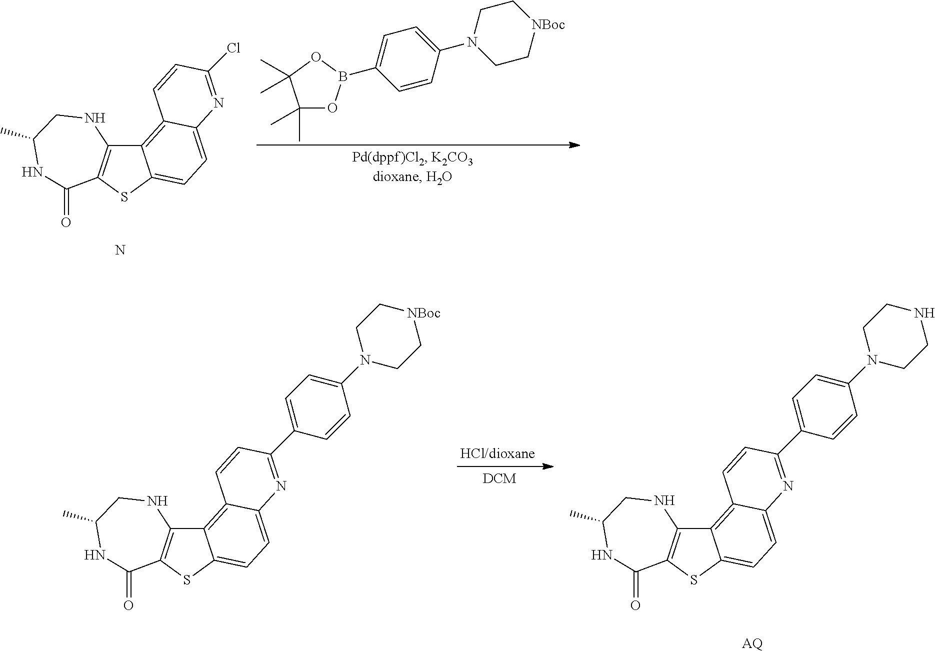

Step 1—(R)-tert-butyl 4-(4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. To a solution of (15R)-5-chloro-15-methyl-11-thia-6,14,17-triazatetracyclo[8.8.0.0^{2,7}.012, is]octadeca-1,3,5,7,9,12(18)-hexaen-13-one (1 g, 3 mmol, Intermediate N) in dioxane (16 mL) and H₂O (4 mL) was added tert-butyl 4-[4-(4,4,5,5-tetramethyl-1,3,2-dioxaborolan-2-yl)phenyl]piperazine-1-carboxylate (1.47 g, 3.78 mmol, CAS #470478-90-1), K₂CO₃ (1.30 g, 9.44 mmol) and Pd(dppf)Cl₂ (345 mg, 472 umol). The mixture was degassed and purged with N₂ three times. Then the mixture was stirred at 80° C. for 2 hrs under N₂ atmosphere. On completion, the reaction mixture was quenched with H₂O (20 mL) at 25° C., and then triturated with EtOAc (100 mL) and filtered to give the compound (2.27 g) as a brown solid. LC-MS (ESI⁺) m/z 544.2. (M+H)⁺.

Step 2—(R)-10-methyl-3-(4-(piperazin-1-yl)phenyl)-9,10,11,12-tetrahydro-8H-[1,4]diazepino[5′,6′:4,5]thieno[3,2-f]quinolin-8-one. To a solution of tert-butyl 4-[4-[(15R)-15-methyl-13-oxo-11-thia-6,14,17-triazatetracyclo[8.8.0.0^{2,7}.0^{12,18}]octadeca-1,3,5,7,9,12(18)-hexaen-5-yl]phenyl]piperazine-1-carboxylate (2.27 g, 4.18 mmol) in DCM (40 mL) was added HCl/dioxane (4 M, 1.04 mL). The reaction was stirred at 25° C. for 1 hr. On completion, the reaction mixture was concentrated under reduced pressure to give a residue. The residue was purified by prep-HPLC (neutral condition) to give a compound (1 g, 47% yield) as a yellow solid. LC-MS (ESI⁺) m/z 444.1. (M+H)⁺.

### 2-(2-(2-fluoro-4-(piperazin-1-yl)phenyl)pyridin-4-yl)-6,7-dihydro-1H-pyrrolo[3,2-c]pyridin-4(5H)-one (Intermediate AR)

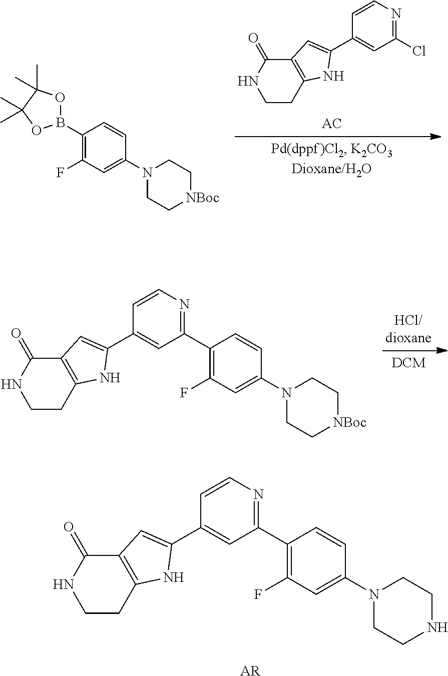 [SMILES: CC(C)(C)OC(=O)N1CCN(c2ccc(B3OC(C)(C)C(C)(C)O3)c(F)c2)CC1 ; O=C1NCCc2[nH]c(-c3ccnc(Cl)c3)cc21 ; CC(C)(C)OC(=O)N1CCN(c2ccc(-c3cc(-c4cc5c([nH]4)CCNC5=O)ccn3)c(F)c2)CC1 ; O=C1NCCc2[nH]c(-c3ccnc(-c4ccc(N5CCNCC5)cc4F)c3)cc21 ; "AR"]

Step 1—Tert-butyl 4-(3-fluoro-4-(4-(4-oxo-4,5,6,7-tetrahydro-1H-pyrrolo[3,2-c]pyridin-2-yl)pyridin-2-yl)phenyl)piperazine-1-carboxylate. To a solution of 2-(2-chloro-4-pyridyl)-1,5,6,7-tetrahydropyrrolo[3,2-c]pyridin-4-one (500 mg, 2.02 mmol, Intermediate AC), in dioxane (10 mL) and H₂O (2 mL) was added tert-butyl 4-[3-fluoro-4-(4,4,5,5-tetramethyl-1,3,2-dioxaborolan-2-yl)phenyl]piperazine-1-carboxylate (1.07 g, 2.62 mmol, CAS #1146950-53-9), Pd(dppf)Cl₂ (148 mg, 202 umol) and K₂CO₃ (837 mg, 6.06 mmol). The reaction was degassed and purged with N₂ three times, and then the mixture was stirred at 80° C. for 2 hrs under N₂ atmosphere. On completion, the mixture was filtered and concentrated under reduced pressure to give a residue. The crude product was triturated with (PE:EA=5/1) at 25° C. for 10 min to give the title compound (900 mg, 75% yield) as a gray solid. LC-MS (ESI+) m/z 492.1 (M+H)⁺.

Step 2 2-(2-(2-Fluoro-4-(piperazin-1-yl)phenyl)pyridin-4-yl)-6,7-dihydro-1H-pyrrolo[3,2-c]pyridin-4(5H)-one. To a solution of tert-butyl 4-[3-fluoro-4-[4-(4-oxo-1,5,6,7-tetrahydropyrrolo[3,2-c]pyridin-2-yl)-2-pyridyl]phenyl]piperazine-1-carboxylate (500 mg, 1.02 mmol) in DCM (10 mL) was added HCl/dioxane (4 M, 10.0 mL). The mixture was stirred at 25° C. for 1 hr. On completion, the reaction mixture was filtered and the filter cake was concentrated under reduced pressure to give a residue. The residue was triturated with DCM at 25° C. for 20 min to give the title compound (2 g, 53% yield) as a brown solid. LC-MS (ESI⁺) m/z 392.1 (M+H)⁺.

### 2′-(2-(2-fluoro-4-(piperazin-1-yl)phenyl)pyridin-4-yl)-5′,6′-dihydrospiro[cyclopentane-1,7′-pyrrolo[3,2-c]pyridin]-4′(1′H)-one (Intermediate AS)

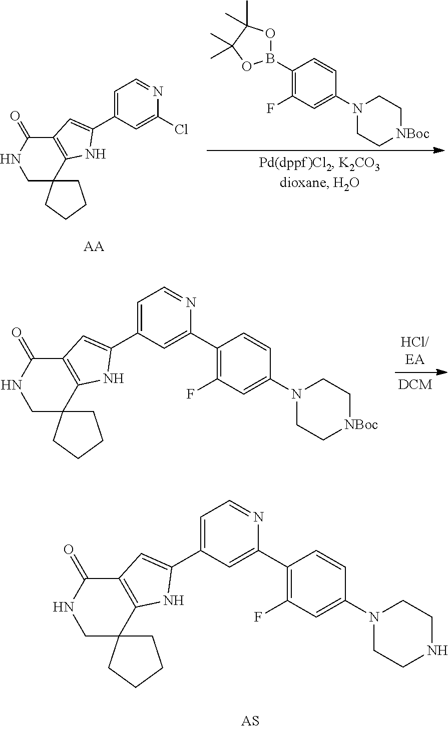

Step 1—Tert-butyl 4-(3-fluoro-4-(4-(4′-oxo-1′,4′,5′,6′-tetrahydrospiro[cyclopentane-1,7′-pyrrolo[3,2-c]pyridin]-2′-yl)pyridin-2-yl)phenyl)piperazine-1-carboxylate. To a solution of 2-(2-chloro-4-pyridyl)spiro[5,6-dihydro-1H-pyrrolo[3,2-c]pyridine-7,1′-cyclopentane]-4-one (180 mg, 596 umol, Intermediate AA) in dioxane (6 mL) and H₂O (1.5 mL) was added tert-butyl 4-[3-fluoro-4-(4,4,5,5-tetramethyl-1,3,2-dioxaborolan-2-yl)phenyl]piperazine-1-carboxylate (364 mg, 895 umol, CAS #1146950-53-9), K₂CO₃ (247 mg, 1.79 mmol) and Pd(dppf)Cl₂ (43.7 mg, 59.7 umol). The reaction was stirred at 80° C. for 2 hrs under N₂ atmosphere. On completion, the reaction mixture was concentrated under reduced pressure to give a residue. The crude product was triturated with (PE/EA=10/1) at 25° C. for 30 min to give the title compound (330 mg, 78% yield) as a brown solid. LC-MS (ESI⁺) m/z 546.3 (M+H)⁺.

Step 2—2′-(2-(2-Fluoro-4-(piperazin-1-yl)phenyl)pyridin-4-yl)-5′,6′-dihydrospiro[cyclopentane-1,7′-pyrrolo[3,2-c]pyridin]-4′(1′H)-one. To a solution of tert-butyl 4-[3-fluoro-4-[4-(4-oxospiro[5,6-dihydro-1H-pyrrolo[3,2-c]pyridine-7,1′-cyclopentane]-2-yl)-2-pyridyl]phenyl]piperazine-1-carboxylate (330 mg, 605 umol) in DCM (8 mL) was added HCl/dioxane (4 M, 151 uL). The reaction was stirred at 25° C. for 1 hr. On completion, the reaction mixture was concentrated under reduced pressure to give the title compound (260 mg) as a yellow solid. LC-MS (ESI⁺) m/z 445.9 (M+H)⁺.

### 2′-(2-(2-Fluoro-4-(piperazin-1-yl)phenyl)pyridin-4-yl)-5′,6′-dihydrospiro[cyclopropane-1,7′-pyrrolo[3,2-c]pyridin]-4′(1′H)-one (Intermediate AT)

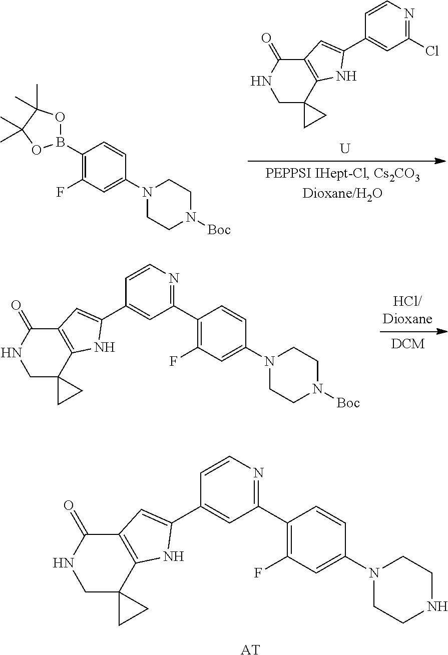

Step 1—Tert-butyl 4-(3-fluoro-4-(4-(4′-oxo-1′,4′,5′,6′-tetrahydrospiro[cyclopropane-1,7′-pyrrolo[3,2-c]pyridin]-2′-yl)pyridin-2-yl)phenyl)piperazine-1-carboxylate. To a solution of 2′-(2-chloropyridin-4-yl)-5′,6′-dihydrospiro[cyclopropane-1,7′-pyrrolo[3,2-c]pyridin]-4′(1′H)-one (80 mg, 292 umol, Intermediate U) and tert-butyl 4-(3-fluoro-4-(4,4,5,5-tetramethyl-1,3,2-dioxaborolan-2-yl)phenyl)piperazine-1-carboxylate (154 mg, 379 umol, CAS #1146950-53-9) in dioxane (4 mL) and H₂O (1 mL) was added Cs₂CO₃ (285 mg, 876 umol) and 1,3-bis[2,6-bis(1-propylbutyl)phenyl]-4,5-dichloro-2H-imidazol-1-ium-2-ide 3-chloropyridine; dichloropalladium (28.4 mg, 29.2 umol). The mixture was stirred at 80° C. for 2 hrs under N₂ atmosphere. The reaction mixture was diluted with H₂O (10 mL) and extracted with EA (3×10 mL). The combined organic layers were dried over Na₂SO₄, filtered and concentrated under reduced pressure to give the title compound (150 mg) as a brown solid. LC-MS (ESI⁺) m/z 518.4 (M+1)⁺.

Step 2—2′-(2-(2-Fluoro-4-(piperazin-1-yl)phenyl)pyridin-4-yl)-5′,6′-dihydrospiro[cyclopropane-1,7-pyrrolo[3,2-c]pyridin]-4′(1′H)-one. To a solution of tert-butyl 4-(3-fluoro-4-(4-(4′-oxo-1′,4′,5′,6′-tetrahydrospiro[cyclopropane-1,7′-pyrrolo[3,2-c]pyridin]-2′-yl)pyridin-2-yl)phenyl)piperazine-1-carboxylate (150 mg, 289 umol) in DCM (10 mL) was added HCl/dioxane (4 M, 3.00 mL). The mixture was stirred at 25° C. for 0.5 hr. On completion, the reaction mixture was diluted with H₂O (30 mL) and extracted with EA (3×30 mL). The combined organic layers were dried over Na₂SO₄, filtered and the filtrate was concentrated under reduced pressure to give the title compound (160 mg, HCl) as a yellow solid. LC-MS (ESI⁺) m/z 418.1 (M+1)⁺.

### 2-(2-chloropyrimidin-4-yl)-6,7-dihydro-1H-pyrrolo[3,2-c]pyridin-4(5H)-one (Intermediate AU)

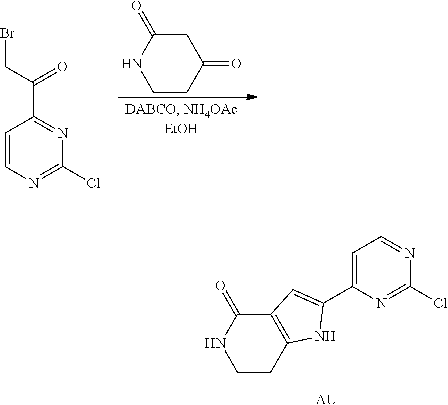

To a solution of piperidine-2,4-dione (1.06 g, 9.34 mmol, CAS #50607-30-2) and 2-bromo-1-(2-chloropyrimidin-4-yl)ethanone (2 g, 8.49 mmol, synthesized via Step 1-2 of Intermediate A) in EtOH (30 mL) was added DABCO (47.6 mg, 425 umol, 46.7 uL) and NH₄OAc (6.55 g, 84.9 mmol) at 25° C., then the mixture was stirred at 25° C. for 1 hr. On completion, the reaction mixture was filtered and concentrated in vacuo to give the crude residue. The crude product was purified by reversed-phase HPLC (0.1% FA condition) to give the title compound (800 mg, 31% yield) as a yellow solid. LC-MS (ESI⁺) m/z 249.0 (M+H)⁺.

### 2-(2-(4-(piperazin-1-yl)phenyl)pyrimidin-4-yl)-6,7-dihydro-1H-pyrrolo[3,2-c]pyridin-4(5H)-one (Intermediate AV)

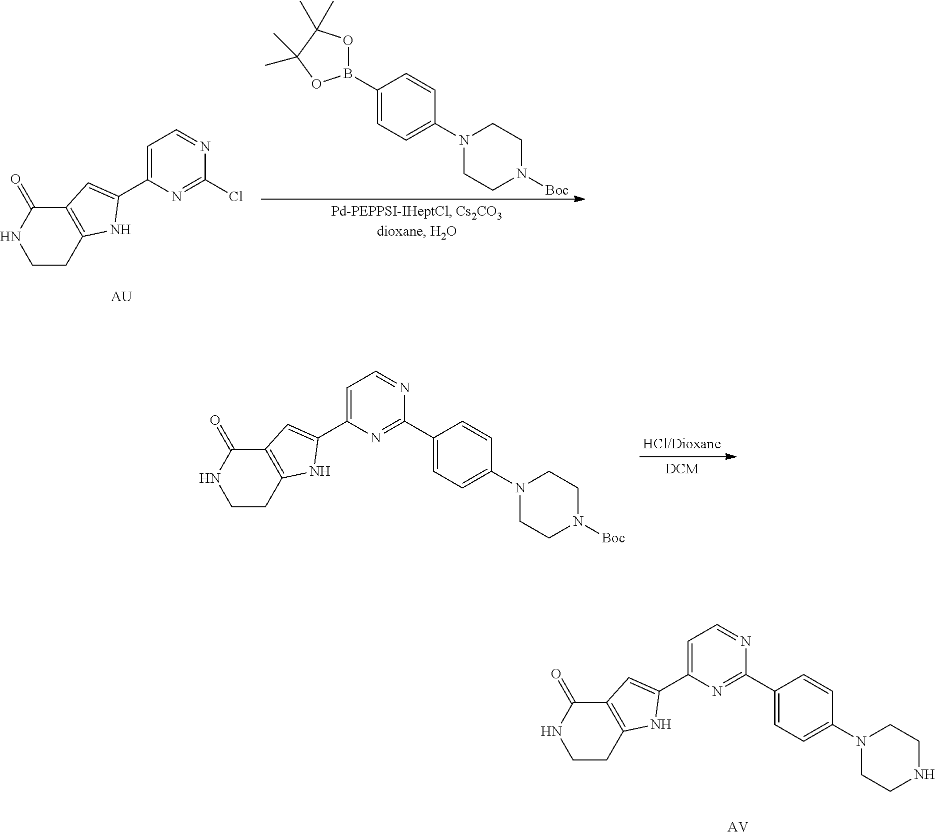

Step 1—Tert-butyl 4-(4-(4-(4-oxo-4,5,6,7-tetrahydro-1H-pyrrolo[3,2-c]pyridin-2-yl)pyrimidin-2-yl)phenyl)piperazine-1-carboxylate. To a solution of tert-butyl 4-(4-(4,4,5,5-tetramethyl-1,3,2-dioxaborolan-2-yl)phenyl)piperazine-1-carboxylate (791 mg, 2.04 mmol, CAS #70478-90-1) and 2-(2-chloropyrimidin-4-yl)-6,7-dihydro-1H-pyrrolo[3,2-c]pyridin-4(5H)-one (500 mg, 1.70 mmol, FA, Intermediate AU) in dioxane (10 mL) and H₂O (2 mL) was added Cs₂CO₃ (1.66 g, 5.09 mmol) and Pd-PEPPSI-IHeptCl (165 mg, 170 umol) at 25° C., then the mixture was stirred at 80° C. for 2 hrs. On completion, the reaction mixture was filtered and the filtrate was concentrated in vacuo to give the crude residue. The residue was purified by column chromatography (SiO₂, Petroleum ether/Ethyl acetate=1/1 to DCM:MeOH=10/1) to give the title compound (50 mg, 62% yield) as a yellow solid. LC-MS (ESI⁺) m/z 475.2. (M+H).

Step 2—2-(2-(4-(Piperazin-1-yl)phenyl)pyrimidin-4-yl)-6,7-dihydro-1H-pyrrolo[3,2-c]pyridin-4(5H)-one. To a solution of tert-butyl 4-(4-(4-(4-oxo-4,5,6,7-tetrahydro-1H-pyrrolo[3,2-c]pyridin-2-yl)pyrimidin-2-yl)phenyl)piperazine-1-carboxylate (110 mg, 232 umol) in DCM (1 mL) was added HCl/dioxane (4 M, 550 uL) at 25° C., then the mixture was stirred at 25° C. for 2 hrs. On completion, the reaction mixture was filtered the filter cake was dried in vacuo to give the title compound (110 mg, HCl) as a yellow solid. LC-MS (ESI⁺) m/z 375.1 (M+H)⁺.

### 2-(4-(1-(4-methoxybenzyl)-2,6-dioxopiperidin-3-yl)phenoxy)ethyl methanesulfonate (Intermediate AW)

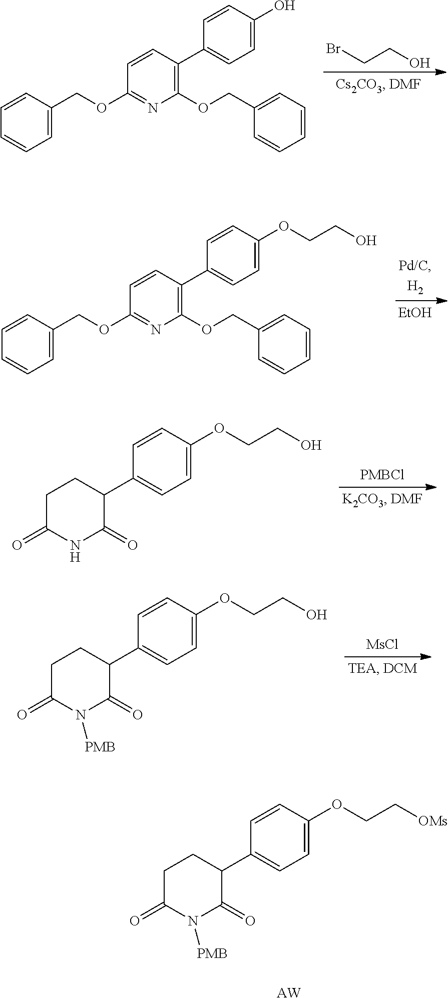

Step 1—2-(4-(2,6-Bis(benzyloxy)pyridin-3-yl)phenoxy)ethanol. To a solution of 4-(2,6-bis(benzyloxy)pyridin-3-yl)phenol (500 mg, 1.30 mmol, CAS #2758531-20-1) in DMF (10 mL) was added Cs₂CO₃ (1.27 g, 3.91 mmol) and 2-bromoethanol (195 mg, 1.56 mmol). The mixture was then stirred at 65° C. for 12 hr. On completion, the reaction mixture was diluted with H₂O (10 mL) and extracted with EA (10 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=10/0 to 5/1) to give the title compound (290 mg, 52% yield) as a yellow oil. LC-MS (ESI⁺) m/z 428.4 (M+H)⁺.

Step 2—3-(4-(2-Hydroxyethoxy)phenyl)piperidine-2,6-dione. A solution of 2-(4-(2,6-bis(benzyloxy)pyridin-3-yl)phenoxy)ethanol (290 mg, 678 umol) in EtOH (4 mL) was added Pd/C (100 mg, 10 wt %), then the reaction was degassed and purged with H₂ three times. Then the mixture was stirred at 20° C. for 2 hr under H₂ (15 psi) atmosphere. On completion, the reaction mixture was filtered and concentrated under reduced pressure to give the title compound (120 mg, 65% yield) as a white solid. LC-MS (ESI⁺) m/z 249.9 (M+H)⁺.

Step 3—3-(4-(2-Hydroxyethoxy)phenyl)-1-(4-methoxybenzyl)piperidine-2,6-dione. To a solution of 3-(4-(2-hydroxyethoxy)phenyl)piperidine-2,6-dione (120 mg, 481 umol) in DMF (4 mL) was added 1-(chloromethyl)-4-methoxy-benzene (84.8 mg, 541 umol) and K₂CO₃ (199 mg, 1.44 mmol). The mixture was stirred at 40° C. for 12 hrs. On completion, the reaction mixture was diluted with H₂O (10 mL) and extracted with EA (10 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=10/0 to 3/1) to give the title compound (95 mg, 50% yield) as a white solid. LC-MS (ESI⁺) m/z 369.9 (M+H)⁺.

Step 4—2-(4-(1-(4-Methoxybenzyl)-2,6-dioxopiperidin-3-yl)phenoxy)ethyl methanesulfonate. To a solution of 3-(4-(2-hydroxyethoxy)phenyl)-1-(4-methoxybenzyl)piperidine-2,6-dione (95 mg, 257 umol) in DCM (3 mL) was added TEA (78 mg, 771 umol) and then MsCl (38.3 mg, 334 umol) was added at 0° C. Then the mixture was stirred at 20° C. for 12 hrs. On completion, the reaction mixture was quenched with H₂O (10 mL) at 0° C., and extracted with EA (10 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=10/0 to 0/10) to give the title compound (55 mg, 37% yield) as a white oil. LC-MS (ESI⁺) m/z 448.1 (M+H)⁺.

### 2,2-dimethoxy-7-azaspiro[3.5]nonane (Intermediate AX)

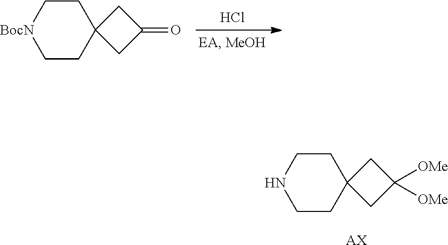

To a solution of tert-butyl 2-oxo-7-azaspiro[3.5]nonane-7-carboxylate (20 g, 80 mmol, CAS #203661-69-2) in EA (30 mL) and MeOH (30 mL) was added HCl (12 M, 13.9 mL). The mixture was stirred at 25° C. for 4 hrs. On completion, the reaction mixture was concentrated under reduced pressure to give the title compound (16 g, HCl) as a white solid. ¹H NMR (400 MHz, DMSO-d₆) δ=3.03 (s, 6H), 2.95-2.90 (m, 4H), 1.93 (s, 4H), 1.74-1.68 (m, 4H). LC-MS (ESI⁺) m/z 185.9. (M+H)⁺.

### 3-(3-methyl-2-oxo-5-(2-oxo-7-azaspiro[3.5]nonan-7-yl)-2,3-dihydro-1H-benzo[d]imidazol-1-yl)piperidine-2,6-dione (Intermediate AY)

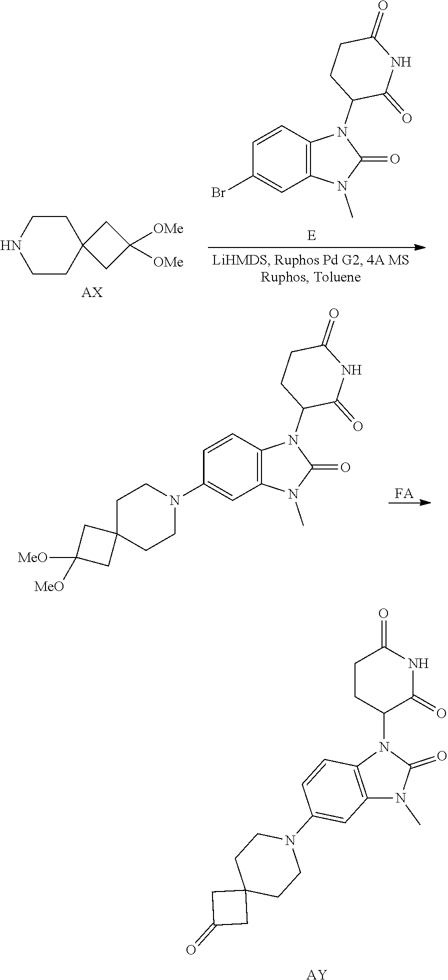

Step 1—3-(5-(2,2-Dimethoxy-7-azaspiro[3.5]nonan-7-yl)-3-methyl-2-oxo-2,3-dihydro-1H-benzo[d]imidazol-1-yl)piperidine-2,6-dione. A mixture of 3-(5-bromo-3-methyl-2-oxo-2,3-dihydro-1H-benzo[d]imidazol-1-yl)piperidine-2,6-dione (16 g, 47.3 mmol, Intermediate E), 2,2-dimethoxy-7-azaspiro[3.5]nonane (12.1 g, 54.4 mmol, HCl, Intermediate AX), LiHMDS (1 M, 284 mL), Ruphos (1.10 g, 2.37 mmol), [2-(2-aminophenyl)phenyl]-chloro-palladium;dicyclohexyl-[2-(2,6-diisopropoxyphenyl)phenyl]phosphane (1.84 g, 2.37 mmol) and 4 Å molecular sieves (1 g) in toluene (200 mL) was degassed and purged with N₂ three times. Then the mixture was stirred at 100° C. for 2 hrs under N₂ atmosphere. On completion, FA was added to the mixture until the pH 6. The mixture was filtered and concentrated under reduced pressure to give a residue. The residue was purified by column chromatography (SiO₂, Petroleum ether/Ethyl acetate=10/1 to 0/1) to give the title compound (12.5 g, 50% yield) as a yellow solid. ¹H NMR (400 MHz, DMSO-d₆) δ=11.05 (s, 1H), 7.97 (dd, J=1.2, 13.6 Hz, 1H), 7.57-7.30 (m, 1H), 7.27-7.04 (m, 1H), 6.92 (d, J=8.8 Hz, 1H), 6.82 (d, J=2.0 Hz, 1H), 6.63 (dd, J=2.0, 8.8 Hz, 1H), 5.28 (dd, J=5.2, 12.8 Hz, 1H), 3.32 (s, 3H), 3.04 (s, 6H), 3.02-2.98 (m, 4H), 2.93-2.82 (m, 2H), 2.70-2.61 (m, 2H), 1.90 (s, 4H), 1.69-1.63 (m, 4H). LC-MS (ESI⁺) m/z 443.4. (M+H)⁺.

Step 2—3-(3-Methyl-2-oxo-5-(2-oxo-7-azaspiro[3.5]nonan-7-yl)-2,3-dihydro-1H-benzo[d]imidazol-1-yl)piperidine-2,6-dione. 3-(5-(2,2-Dimethoxy-7-azaspiro[3.5]nonan-7-yl)-3-methyl-2-oxo-2,3-dihydro-1H-benzo[d]imidazol-1-yl)piperidine-2,6-dione (11 g, 24.9 mmol) was added to formic acid (4 mL) and the mixture was stirred at 40° C. for 1 hr. On completion, the reaction mixture was concentrated under reduced pressure to give the title compound (10 g) as a yellow oil. LC-MS (ESI⁺) m/z 396.8. (M+H)⁺.

### 2′-(2-(6-(piperazin-1-yl)pyridin-3-yl)pyrimidin-4-yl)-5′,6′-dihydrospiro[cyclopropane-1,7′-pyrrolo[3,2-c]pyridin]-4′(1′H)-one (Intermediate AZ)

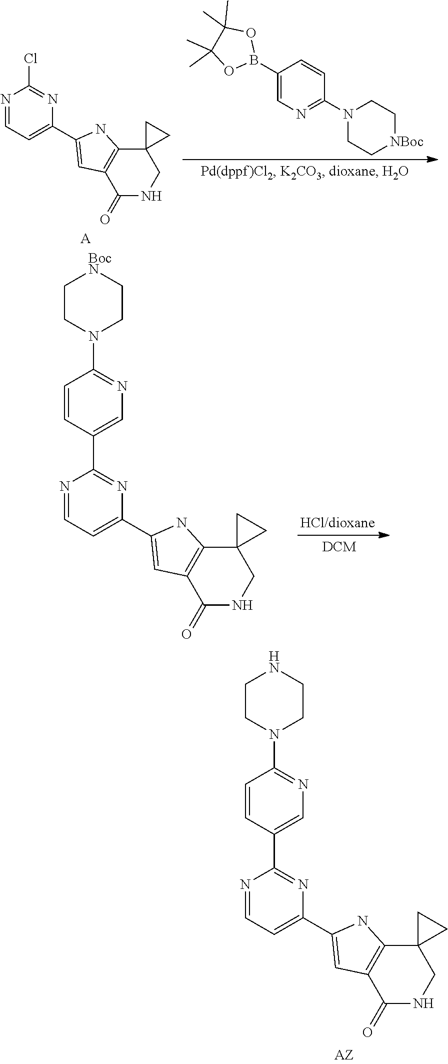

Step 1—Tert-butyl 4-(5-(4-(4′-oxo-1′,4′,5′,6′-tetrahydrospiro[cyclopropane-1,7′-pyrrolo[3,2-c]pyridin]-2′-yl)pyrimidin-2-yl)pyridin-2-yl)piperazine-1-carboxylate. A mixture of 2′-(2-chloropyrimidin-4-yl)-5′,6′-dihydrospiro[cyclopropane-1,7′-pyrrolo[3,2-c]pyridin]-4′(1′H)-one (300 mg, 1.09 mmol, Intermediate A), tert-butyl 4-(5-(4,4,5,5-tetramethyl-1,3,2-dioxaborolan-2-yl)pyridin-2-yl)piperazine-1-carboxylate (424 mg, 1.09 mmol, CAS #496786-98-2), Pd(dppf)Cl₂ (79 mg, 109 umol), and K₂CO₃ (451 mg, 3.27 mmol) in dioxane (5 mL) and H₂O (2 mL) was degassed and purged with N₂ three times. Then the mixture was stirred at 80° C. for 2 hrs under N₂ atmosphere. On completion, the reaction mixture was concentrated under reduced pressure to give the title compound (500 mg) as a black solid. LC-MS (ESI+) m/z 502.3 (M+H)⁺.

Step 2—2′-(2-(6-(piperazin-1-yl)pyridin-3-yl)pyrimidin-4-yl)-5′,6′-dihydrospiro[cyclopropane-1,7′-pyrrolo[3,2-c]pyridin]-4′(1′H)-one. To a solution of tert-butyl 4-(5-(4-(4′-oxo-1′,4′,5′,6′-tetrahydrospiro[cyclopropane-1,7′-pyrrolo[3,2-c]pyridin]-2′-yl)pyrimidin-2-yl)pyridin-2-yl)piperazine-1-carboxylate (200 mg, 398 umol) in DCM (3 mL) was added HCl/dioxane (4 M, 99.6 uL), then the mixture was stirred at 25° C. for 1 hr. On completion, the reaction mixture was concentrated under reduced pressure to give the title compound (160 mg) as a red solid. LC-MS (ESI⁺) m/z 402.0 (M+H)⁺.

### 2-((tert-butyldimethylsilyl)oxy)-7-azaspiro[3.5]nonane (Intermediate BA)

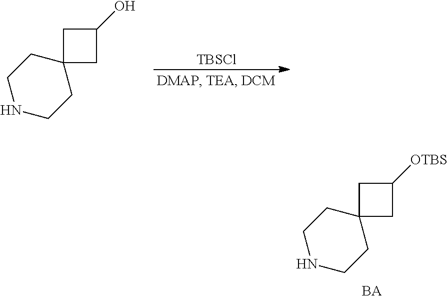 [SMILES: OC1CC2(CCNCC2)C1 ; "TBSCl" ; CC(C)(C)[Si](C)(C)OC1CC2(CCNCC2)C1]

To a solution of 7-azaspiro[3.5]nonan-2-ol (5 g, 35.4 mmol, CAS #784137-09-3) in DCM (100 mL) was added TEA (10.8 g, 106 mmol, 14.8 mL), DMAP (433 mg, 3.54 mmol) and TBSCl (5.87 g, 39.0 mmol). The mixture was stirred at 25° C. for 12 hrs. On completion, the reaction mixture was filtered and quenched with H₂O (100 mL) and then extracted with DCM (50 mL×2). The combined organic layers were dried over Na₂SO₄, filtered and concentrated under reduced pressure to give the title compound (7.5 g) as a yellow oil. LC-MS (ESI⁺) m/z 256.1. (M+H)⁺.

### 3-(3-methyl-2-oxo-4-(2-oxo-7-azaspiro[3.5]nonan-7-yl)-2,3-dihydro-1H-benzo[d]imidazol-1-yl)piperidine-2,6-dione (Intermediate BB)

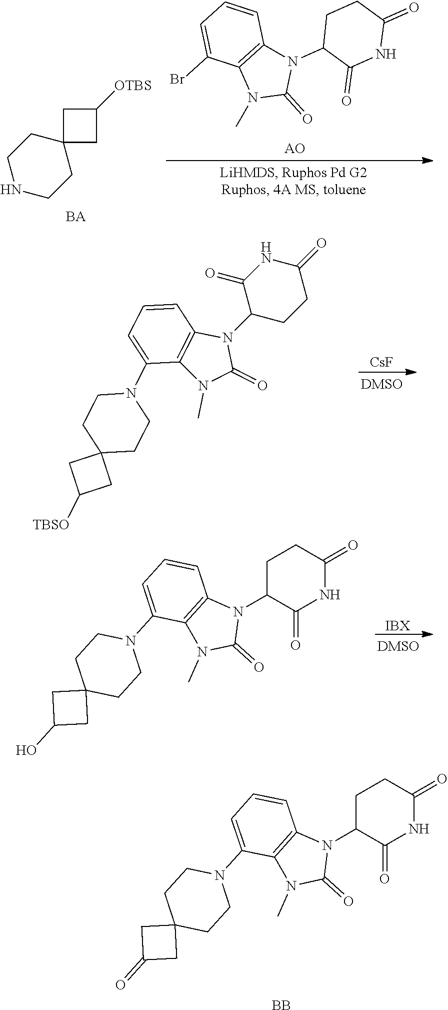

Step 1—3-(4-(2-((Tert-butyldimethylsilyl)oxy)-7-azaspiro[3.5]nonan-7-yl)-3-methyl-2-oxo-2,3-dihydro-1H-benzo[d]imidazol-1-yl)piperidine-2,6-dione. A mixture of 3-(4-bromo-3-methyl-2-oxo-2,3-dihydro-1H-benzo[d]imidazol-1-yl)piperidine-2,6-dione (1.5 g, 4.4 mmol, Intermediate AO), 2-((tert-butyldimethylsilyl)oxy)-7-azaspiro[3.5]nonane (1.70 g, 6.65 mmol, Intermediate BA), LiHMDS (1 M, 26.6 mL), Ruphos (103.50 mg, 221.79 umol), Ruphos Pd G2 (172 mg, 222 umol) and 4A molecular sieves (500 mg) in toluene (50 mL) was degassed and purged with N₂ three times. Then the mixture was stirred at 100° C. for 2 hrs under N₂ atmosphere. On completion, FA was added to the mixture until the pH 6. The mixture was filtered and concentrated under reduced pressure to give a residue (2.9 g) as a yellow solid. LC-MS (ESI⁺) m/z 513.2. (M+H)⁺.

Step 2—3-(4-(2-Hydroxy-7-azaspiro[3.5]nonan-7-yl)-3-methyl-2-oxo-2,3-dihydro-1H-benzo[d]imidazol-1-yl)piperidine-2,6-dione. To a solution of 3-(4-(2-((tert-butyldimethylsilyl)oxy)-7-azaspiro[3.5]nonan-7-yl)-3-methyl-2-oxo-2,3-dihydro-1H-benzo[d]imidazol-1-yl)piperidine-2,6-dione (1 g, 1.95 mmol) in DMSO (10 mL) was added CsF (889 mg, 5.85 mmol). The mixture was stirred at 50° C. for 2 hrs. On completion, the reaction mixture was filtered and concentrated under reduced pressure to give a residue. The residue was purified by prep-HPLC (0.1% FA) to give the title compound (400 mg, 45% yield, FA) as a white solid. LC-MS (ESI⁺) m/z 398.8. (M+H)⁺.

Step 3—3-(3-Methyl-2-oxo-4-(2-oxo-7-azaspiro[3.5]nonan-7-yl)-2,3-dihydro-1H-benzo[d]imidazol-1-yl)piperidine-2,6-dione. To a solution of 3-(4-(2-hydroxy-7-azaspiro[3.5]nonan-7-yl)-3-methyl-2-oxo-2,3-dihydro-1H-benzo[d]imidazol-1-yl)piperidine-2,6-dione (100 mg, 251 umol) in DMSO (2 mL) was added IBX (141 mg, 502 umol) and the mixture was stirred at 25° C. for 1 hr. On completion, the reaction mixture was filtered and concentrated under reduced pressure to give the title compound (300 mg) as a yellow oil. LC-MS (ESI⁺) m/z 397.1. (M+H)⁺.

### 1-(3-(2,6-Dioxopiperidin-3-yl)phenyl)piperidine-4-carbaldehyde (Intermediate BC)

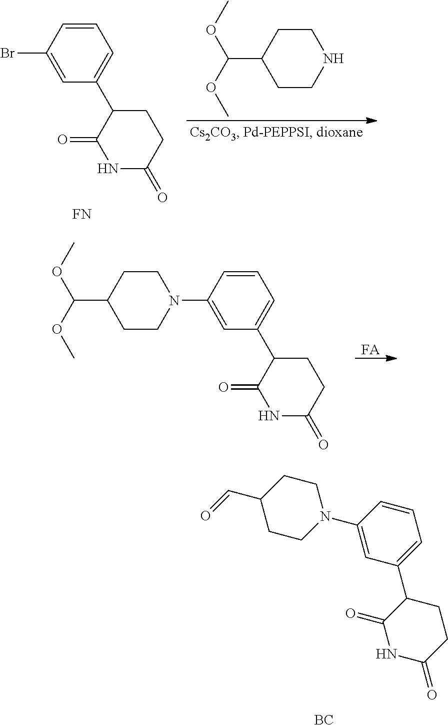

Step 1—3-(3-(4-(Dimethoxymethyl)piperidin-1-yl)phenyl)piperidine-2,6-dione. A mixture of 3-(3-bromophenyl)piperidine-2,6-dione (500 mg, 1.86 mmol, Intermediate FN), 4-(dimethoxymethyl)piperidine (445 mg, 2.80 mmol, CAS #188646-83-5), Cs₂CO₃ (1.82 g, 5.59 mmol), 1,3-bis[2,6-bis(1-propylbutyl)phenyl]-4,5-dichloro-2H-imidazol-1-ium-2-ide 3-chloropyridine dichloropalladium (181 mg, 186 umol) in dioxane (1 mL) was degassed and purged with N₂ three times. Then the mixture was stirred at 100° C. for 3 hrs under N₂ atmosphere. On completion, the reaction mixture was filtered and the filtrate was purified by prep-HPLC (FA condition) to give the title compound (100 mg, 9% yield, FA) as a black solid. LC-MS (ESI⁺) m/z 347.0 (M+H)⁺.

Step 2—1-(3-(2,6-Dioxopiperidin-3-yl)phenyl)piperidine-4-carbaldehyde. 3-(3-(4-(dimethoxymethyl) piperidin-1-yl) phenyl) piperidine-2, 6-dione (50 mg, 144 umol) was added to FA (1 mL) and the mixture was stirred at 25° C. for 2 hrs. On completion, the reaction mixture was concentrated under reduced pressure to give the title compound (40 mg) as a black solid. LC-MS (ESI⁺) m/z 319.2 (M+H)⁺.

### 1-(4-(2,6-dioxopiperidin-3-yl)phenyl)piperidine-4-carbaldehyde (Intermediate BD)

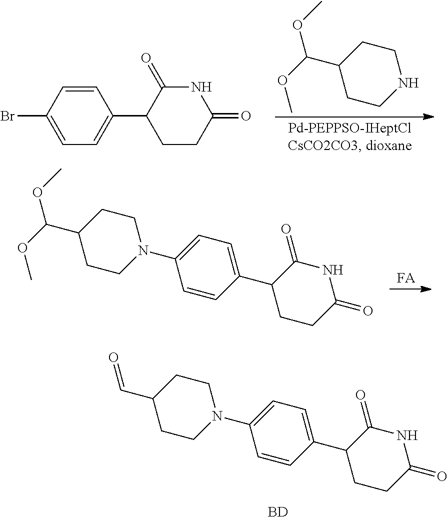

Step 1—3-(4-(4-(Dimethoxymethyl)piperidin-1-yl)phenyl)piperidine-2,6-dione. A mixture of 3-(4-bromophenyl)piperidine-2,6-dione (300 mg, 1.12 mmol, CAS #1267337-47-2), 4-(dimethoxymethyl)piperidine (285 mg, 1.79 mmol, CAS #188846-83-5) in dioxane (1 mL) was added Cs₂CO₃ (1.09 g, 3.36 mmol) and Pd-PEPPSI-IHeptCl (108 mg, 111 umol) at 25° C., and then the mixture was stirred at 25-100° C. for 2 hours under N₂ atmosphere. On completion, the reaction mixture was filtered and concentrated in vacuo to give the crude residue. The residue was purified by column chromatography (SiO₂, Petroleum ether/Ethyl acetate=0/1) to give the title compound (300 mg, 44% yield) as white solid. LC-MS (ESI+) m/z 347.2. (M+H)⁺.

Step 2—1-(4-(2,6-Dioxopiperidin-3-yl)phenyl)piperidine-4-carbaldehyde. 3-(4-(4-(Dimethoxymethyl)piperidin-1-yl)phenyl)piperidine-2,6-dione (150 mg, 433 umol) was added in HCOOH (2 mL) and the mixture was stirred at 25° C. for 2 hrs. On completion, the reaction mixture was filtered and concentrated in vacuo to give the title compound (100 mg) as red oil. LC-MS (ESI⁺) m/z 319.1. (M+H)⁺.

### (R)-3-((1R,4R)-2,5-diazabicyclo[2.2.2]octan-2-yl)-10-methyl-9,10,11,12-tetrahydro-8H-[1,4]diazepino[5′,6′:4,5]thieno[3,2-f]quinolin-8-one (Intermediate BE)

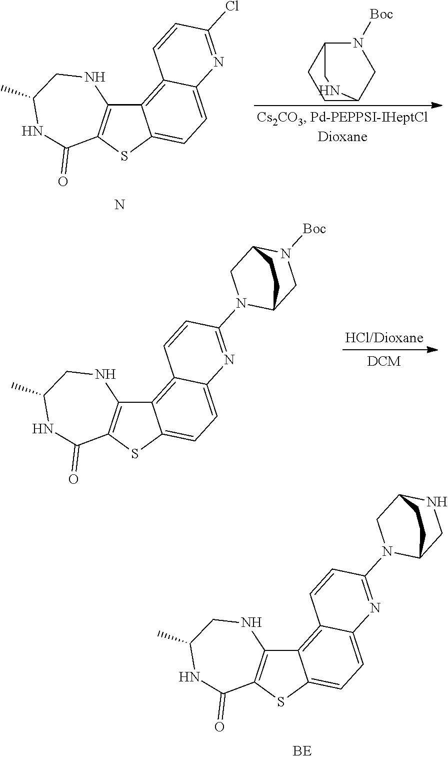

Step 1—(1R,4R)-tert-butyl 5-((R)-10-methyl-8-oxo-9,10,11,12-tetrahydro-8H-[1,4]diazepino[5′,6′:4,5]thieno[3,2-f]quinolin-3-yl)-2,5-diazabicyclo[2.2.2]octane-2-carboxylate. To a mixture of (R)-3-chloro-10-methyl-9,10,11,12-tetrahydro-8H-[1,4]diazepino[5′,6′:4,5]thieno[3,2-f]quinolin-8-one (600 mg, 1.89 mmol, Intermediate N), tert-butyl 2,5-diazabicyclo[2.2.2]octane-2-carboxylate (801 mg, 3.78 mmol, CAS #858671-91-7), Cs₂CO₃ (1.85 g, 5.66 mmol), and Pd-PEPPSI-IHeptCl (183 mg, 188 umol) in dioxane (12 mL) was degassed and purged with N₂ three times. Then the mixture was stirred at 100° C. for 4 hr under N₂ atmosphere. On completion, the reaction mixture was diluted with H₂O (20 mL) and extracted with DCM (20 mL×3). The combined organic layers were washed with brine (20 mL×3), dried over Na₂SO₄, filtered and concentrated under reduced pressure to give a residue. The residue was purified by prep-HPLC (FA condition) to give a yellow solid, which was further separated by SFC (column: DAICEL CHIRALPAK AS (250 mm×30 mm, 10 um); mobile phase: [0.1% NH₃H₂O MEOH]; B %: 35%-35%, A 3.5; 50 min) to give the title compound (120 mg, 40% yield) as a yellow solid. LC-MS (ESI⁺) m/z 494.2 (M+H)⁺.

Step 2—(R)-3-((1R,4R)-2,5-diazabicyclo[2.2.2]octan-2-yl)-10-methyl-9,10,11,12-tetrahydro-8H-[1,4]diazepino[5′,6′:4,5]thieno[3,2-f]quinolin-8-one. To a solution of (1R,4R)-tert-butyl 5-((R)-10-methyl-8-oxo-9,10,11,12-tetrahydro-8H-[1,4]diazepino[5′,6′:4,5]thieno[3,2-f]quinolin-3-yl)-2,5-diazabicyclo[2.2.2]octane-2-carboxylate (120 mg, 243 umol) in DCM (2 mL) was added HCl/dioxane (4 M, 607 uL). The mixture was stirred at 25° C. for 1 hr. On completion, the reaction mixture was concentrated under reduced pressure to give the title compound (95 mg) as a yellow solid. LC-MS (ESI⁺) m/z 393.9 (M+H)⁺.

### 1-(4-hydroxyphenyl)-3-(4-methoxybenzyl)dihydropyrimidine-2,4(1H,3H)-dione (Intermediate BF)

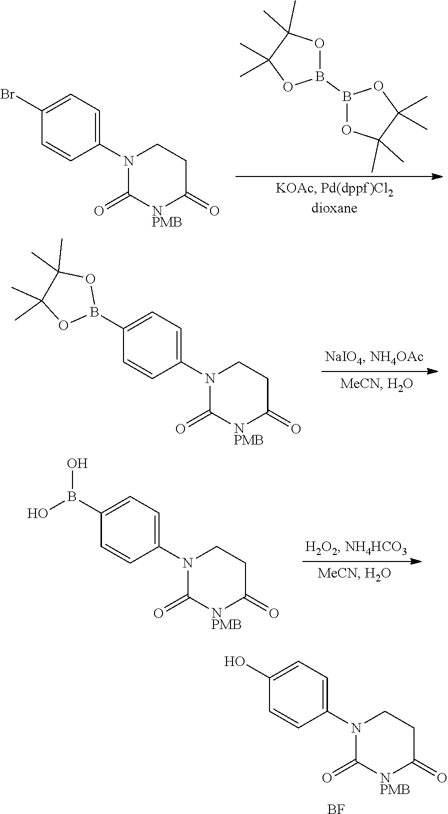

Step 1—3-(4-Methoxybenzyl)-1-(4-(4,4,5,5-tetramethyl-1,3,2-dioxaborolan-2-yl)phenyl)dihydropyrimidine-2,4(1H,3H)-dione. To a solution of 1-(4-bromophenyl)-3-[(4-methoxyphenyl)methyl]hexahydropyrimidine-2,4-dione (500 mg, 1.28 mmol, Intermediate FO) in dioxane (10 mL) was added 4,4,5,5-tetramethyl-2-(4,4,5,5-tetramethyl-1,3,2-dioxaborolan-2-yl)-1,3,2-dioxaborolane (1.30 g, 5.14 mmol, CAS #73183-34-3), KOAc (378 mg, 3.85 mmol) and Pd(dppf)Cl₂ (94.0 mg, 128 umol). The reaction was stirred at 80° C. for 12 hrs under N₂ atmosphere. On completion, the reaction mixture was quenched with H₂O (20 mL) at 25° C., and then extracted with EA (40 mL×3). The combined organic layers were washed with brine (40 mL×2), dried over Na₂SO₄, filtered and concentrated under reduced pressure to give a residue. The residue was purified by column chromatography (SiO₂, Petroleum ether/Ethyl acetate=10/1 to 1/1) to give the title compound (620 mg, 72% yield) as a yellow oil. LC-MS (ESI⁺) m/z 437.0 (M+H)⁺.

Step 2—(4-(3-(4-Methoxybenzyl)-2,4-dioxotetrahydropyrimidin-1(2H)-yl)phenyl)boronic acid. To a solution of 3-[(4-methoxyphenyl)methyl]-1-[4-(4,4,5,5-tetramethyl-1,3,2-dioxaborolan-2-yl)phenyl]hexahydropyrimidine-2,4-dione (500 mg, 1.15 mmol) in MeCN (6 mL) and H₂O (4 mL) was added NaIO₄ (735 mg, 3.44 mmol, 190 uL) and NH₄OAc (177 mg, 2.29 mmol). The reaction was stirred at 25° C. for 12 hrs. On completion, the reaction mixture was quenched with Na₂SO₃ solution (20 mL) at 25° C., and then extracted with EA (20 mL×3). The combined organic layers were washed with NaCl (20 mL×2), dried over Na₂SO₄, filtered and concentrated under reduced pressure to give the title compound (350 mg) as a white solid. LC-MS (ESI⁺) m/z 422.0 (M+H)⁺.

Step 3—1-(4-Hydroxyphenyl)-3-(4-methoxybenzyl)dihydropyrimidine-2,4(1H,3H)-dione. To a solution of [4-[3-[(4-methoxyphenyl)methyl]-2,4-dioxo-hexahydropyrimidin-1-yl]phenyl]boronic acid (250 mg, 706 umol) in H₂O (1.5 mL) and MeCN (1.5 mL) was added NH₄HCO₃ (55.80 mg, 706 umol, 58.1 uL) and H₂O₂(160 mg, 1.41 mmol, 136 uL, 30% solution). The reaction was stirred at 20° C. for 2 hrs. On completion, the reaction mixture was quenched with NaHSO₃ 10 mL at 25° C., and then extracted with EA (15 mL×3). The combined organic layers were washed with NaCl (20 mL×2), dried over Na₂SO₄, filtered and concentrated under reduced pressure to give the title compound (260 mg) as a white solid. LC-MS (ESI⁺) m/z 326.9 (M+H)⁺.

### 4-(4-(3-(4-methoxybenzyl)-2,4-dioxotetrahydropyrimidin-1(2H)-yl)phenoxy)butanal (Intermediate BG)

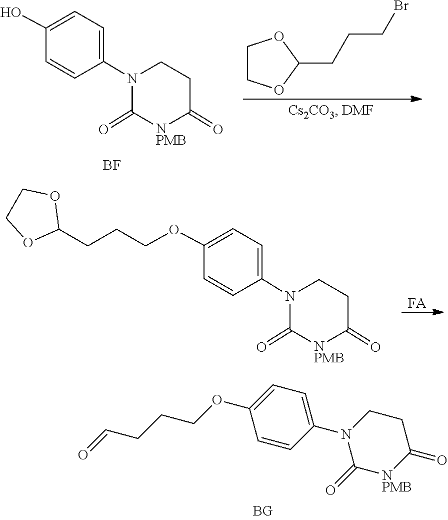

Step 1—1-(4-(3-(1,3-Dioxolan-2-yl)propoxy)phenyl)-3-(4-methoxybenzyl)dihydropyrimidine-2,4(1H,3H)-dione. To a solution of 1-(4-hydroxyphenyl)-3-[(4-methoxyphenyl)methyl]hexahydropyrimidine-2,4-dione (200 mg, 613 umol, Intermediate BF) in DMF (2 mL) was added 2-(3-bromopropyl)-1,3-dioxolane (179 mg, 919 umol) and Cs₂CO₃ (599 mg, 1.84 mmol). The reaction was then stirred at 65° C. for 12 hrs. On completion, the reaction mixture was quenched with H₂O (10 mL) at 25° C., and then extracted with EA (20 mL×3). The combined organic layers were washed with NaCl (15 mL×2), dried over Na₂SO₄, filtered and concentrated under reduced pressure to give a residue. The residue was purified by column chromatography (SiO₂, Petroleum ether/Ethyl acetate=10/1 to 1/1) to give a title compound (230 mg, 85% yield) as a colorless liquid. LC-MS (ESI+) m/z 441.2 (M+H)⁺.

Step 2—4-(4-(3-(4-Methoxybenzyl)-2,4-dioxotetrahydropyrimidin-1(2H)-yl)phenoxy)butanal. A solution of 1-[4-[3-(1,3-dioxolan-2-yl)propoxy]phenyl]-3-[(4-methoxyphenyl)methyl]hexahydropyrimidine-2,4-dione (100 mg, 227 umol) in HCOOH (2 mL) was stirred at 25° C. for 2 hrs. On completion, the reaction mixture was concentrated under reduced pressure to give the title compound (90 mg) as a brown solid. LC-MS (ESI⁺) m/z 397.3 (M+H)⁺.

### 1-(4-(3-(4-methoxybenzyl)-2,4-dioxotetrahydropyrimidin-1(2H)-yl)phenyl)piperidine-4-carbaldehyde (Intermediate BH)

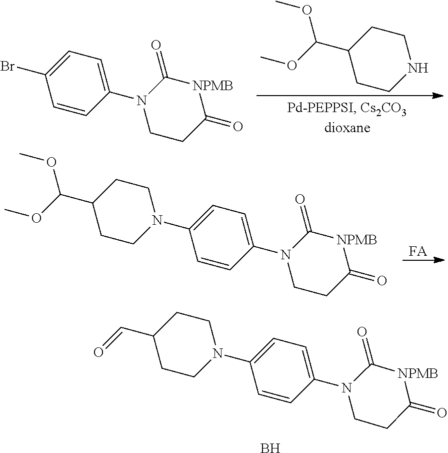

Step 1 1-(4-(4-(Dimethoxymethyl)piperidin-1-yl)phenyl)-3-(4-methoxybenzyl)dihydropyrimidine-2,4(1H,3H)-dione. To a solution of 1-(4-bromophenyl)-3-[(4-methoxyphenyl)methyl]hexahydropyrimidine-2,4-dione (110 mg, 283 umol, Intermediate FO) in dioxane (1 mL) was added 4-(dimethoxymethyl)piperidine (67.5 mg, 424 umol, CAS #188646-83-5), Cs₂CO₃ (276 mg, 848 umol) and 1,3-bis[2,6-bis(1-propylbutyl)phenyl]-4,5-dichloro-2H-imidazol-1-ium-2-ide 3-chloropyridine dichloropalladium (27.5 mg, 28.3 umol). The mixture was stirred at 100° C. for 3 hrs under N₂ atmosphere. On completion, the reaction mixture was concentrated under reduced pressure to give a residue. The residue was purified by column chromatography (SiO₂, Petroleum ether/Ethyl acetate=4/1 to 0/1) to give a title compound (130 mg, 94% yield) as an orange solid. LC-MS (ESI⁺) m/z 468.4 (M+H)⁺.

Step 2—1-(4-(3-(4-Methoxybenzyl)-2,4-dioxotetrahydropyrimidin-1(2H)-yl)phenyl)piperidine-4-carbaldehyde. 1-[4-[4-(dimethoxymethyl)-1-piperidyl]phenyl]-3-[(4-methoxyphenyl)methyl]hexahydropyrimidine-2,4-dione (65 mg, 139 umol) was added in HCOOH (2 mL). The mixture was stirred at 25° C. for 1 hr. On completion, the reaction mixture was concentrated under reduced pressure to give the title compound (60 mg) as a brown solid. LC-MS (ESI⁺) m/z 422.0 (M+H)⁺.

### 3-(3-methyl-2-oxo-4-(4-oxopiperidin-1-yl)-2,3-dihydro-1H-benzo[d]imidazol-1-yl)piperidine-2,6-dione (Intermediate BI)

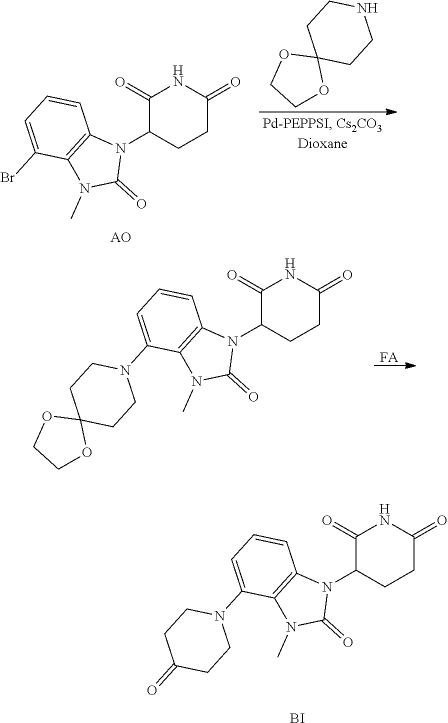

Step 1—3-(3-Methyl-2-oxo-4-(1,4-dioxa-8-azaspiro[4.5]decan-8-yl)-2,3-dihydro-1H-benzo[d]imidazol-1-yl)piperidine-2,6-dione. A mixture of 3-(4-bromo-3-methyl-2-oxo-benzimidazol-1-yl)piperidine-2,6-dione (500 mg, 1.48 mmol, Intermediate AO), 1,4-dioxa-8-azaspiro[4.5]decane (211 mg, 1.48 mmol, CAS #177-11-7), 1,3-bis[2,6-bis(1-propylbutyl)phenyl]-4,5-dichloro-2H-imidazol-1-ium-2-ide;3-chloropyridine dichloropalladium (143 mg, 147 umol) and Cs₂CO₃ (1.45 g, 4.44 mmol) were taken up into a microwave tube in dioxane (20 mL). The sealed tube was heated at 120° C. for 2 hrs. On completion, the reaction mixture was concentrated under reduced pressure to give a residue. The residue was purified by column chromatography (SiO₂, Petroleum ether/Ethyl acetate=1/1 to 0/1) to give the title compound (400 mg, 68% yield) as a yellow solid. LC-MS (ESI⁺) m/z 401.1 (M+H)⁺.

Step 2—3-(3-Methyl-2-oxo-4-(4-oxopiperidin-1-yl)-2,3-dihydro-1H-benzo[d]imidazol-1-yl)piperidine-2,6-dione. 3-[4-(1,4-dioxa-8-azaspiro[4.5]decan-8-yl)-3-methyl-2-oxo-benzimidazol-1-yl]piperidine-2,6-dione (390 mg, 973 umol) was dissolved in formic acid (1 mL) and the mixture was stirred at 25° C. for 4 hrs. On completion, the reaction mixture was concentrated under reduced pressure to give the title compound (300 mg, FA salt) as yellow solid. LC-MS (ESI⁺) m/z 357.1 (M+H)⁺.

### (R)-10-methyl-3-(2,7-diazaspiro[3.5]nonan-7-yl)-9,10,11,12-tetrahydro-8H-[1,4]diazepino[5′,6′:4,5]thieno[3,2-f]quinolin-8-one (Intermediate BJ)

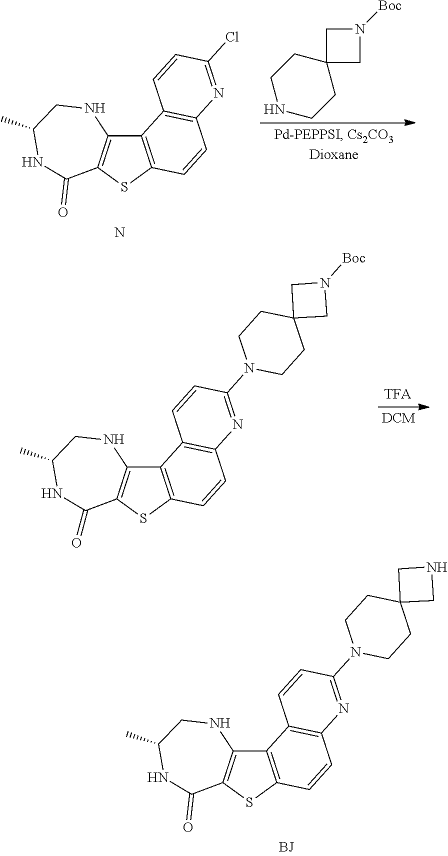

Step 1—(R)-tert-butyl 7-(10-methyl-8-oxo-9,10,11,12-tetrahydro-8H-[1,4]diazepino[5′,6′:4,5]thieno[3,2-f]quinolin-3-yl)-2,7-diazaspiro[3.5]nonane-2-carboxylate. A mixture of (15R)-5-chloro-15-methyl-11-thia-6,14,17-triazatetracyclo[8.8.0.02,7.012,18]octadeca-1(10),2(7),3,5,8,12(18)-hexaen-13-one (300 mg, 944 umol, Intermediate N), tert-butyl 2,7-diazaspiro[3.5]nonane-2-carboxylate (213 mg, 944 umol, CAS #236406-55-6), 1,3-bis[2,6-bis(1-propylbutyl)phenyl]-4,5-dichloro-2H-imidazol-1-ium-2-ide;3-chloropyridine; dichloropalladium (91.8 mg, 94.4 umol, CAS #1814936-54-3), and Cs₂CO₃ (922 mg, 2.83 mmol) in 1,4-dioxane (4 mL) was degassed and purged with N₂ three times. Then the mixture was stirred at 100° C. for 4 hrs under N₂ atmosphere. On completion, the reaction mixture was concentrated under reduced pressure and purified by column chromatography (SiO₂, Petroleum ether/Ethyl acetate=1/1 to 0/1) to give the title compound (200 mg, 42% yield) as a yellow solid. LC-MS (ESI⁺) m/z 508.3 (M+H)⁺.

Step 4—(R)-10-methyl-3-(2,7-diazaspiro[3.5]nonan-7-yl)-9,10,11,12-tetrahydro-8H-[1,4]diazepino[5′,6′:4,5]thieno[3,2-f]quinolin-8-one

To a solution of (R)-tert-butyl 7-(10-methyl-8-oxo-9,10,11,12-tetrahydro-8H-[1,4]diazepino[5′,6′:4,5]thieno[3,2-f]quinolin-3-yl)-2,7-diazaspiro[3.5]nonane-2-carboxylate (120 mg, 236 umol) in DCM (2 mL) was added TFA (308 mg, 2.70 mmol). The mixture was stirred at 25° C. for 4 hrs. On completion, the reaction mixture was concentrated under reduced pressure to give the title compound (100 mg, TFA salt) as yellow oil. LC-MS (ESI⁺) m/z 408.0 (M+H)⁺.

### 6-Azaspiro[3.5]nonane-7,9-dione (Intermediate BK)

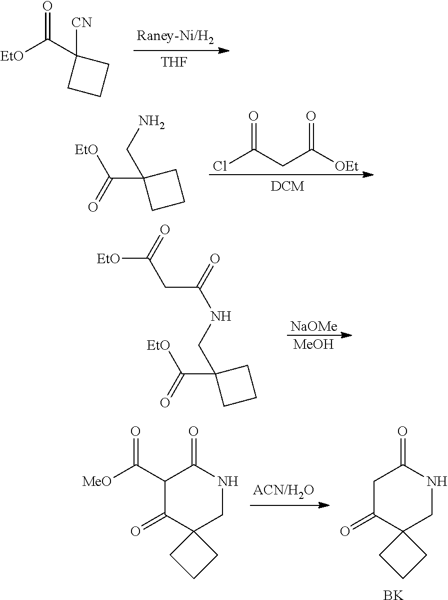

Step 1—Ethyl 1-(aminomethyl)cyclobutanecarboxylate. To a solution of ethyl 1-cyanocyclobutanecarboxylate (3 g, 19.6 mmol, CAS #28246-87-9) in THF (20 mL) was added Raney-Ni (1.68 g, 19.6 mmol) under N₂ atmosphere. The suspension was degassed and purged with H₂ three times. The mixture was then stirred under H₂ (40 Psi) at 25° C. for 3 hrs. On completion, the mixture was filtered to give a filtrate and concentrated under reduced pressure to give the title compound (3.1 g) as a yellow oil. LC-MS (ESI⁺) m/z 158.2 (M+H)⁺.

Step 2—Ethyl 1-((3-ethoxy-3-oxopropanamido)methyl)cyclobutanecarboxylate. To a solution of ethyl 1-(aminomethyl)cyclobutanecarboxylate (3 g, 19.1 mmol) in DCM (40 mL) was added ethyl 3-chloro-3-oxopropanoate (3.45 g, 22.9 mmol, 2.87 mL). The mixture was stirred at 0° C. for 2 hrs. On completion, the mixture was quenched with H₂O (50 mL) and extracted with DCM (30 mL×3). The organic phase was dried over anhydrous Na₂SO₄, then filtered and concentrated under reduced pressure to give a residue. The residue was purified by column chromatography (SiO₂, Petroleum ether/Ethyl acetate=10/1 to 3/1) to give the title compound (500 mg, 94% yield) as a gray solid. LC-MS (ESI⁺) m/z 272.2 (M+H)⁺.

Step 3—Methyl 7,9-dioxo-6-azaspiro[3.5]nonane-8-carboxylate. To a solution of ethyl 1-((3-ethoxy-3-oxopropanamido)methyl)cyclobutanecarboxylate (3.7 g, 14 mmol) in MeOH (30 mL) was added NaOMe (2.70 g, 15.0 mmol, 8 mL, 30% solution). The mixture was stirred at 60° C. for 6 hrs. On completion, the mixture was quenched by adding water (10 mL), then extracted with EtOAc(10 mL×2). Next, 1M HCl solution was added to adjust the pH=5, then extracted with EtOAc (30 mL×3). The organic phase was concentrated under reduced pressure to give the title compound (2 g) as a yellow solid. LC-MS (ESI⁺) m/z 212.0 (M+H)⁺.

Step 4—6-Azaspiro[3.5]nonane-7,9-dione. A solution of methyl 7,9-dioxo-6-azaspiro[3.5]nonane-8-carboxylate (2 g, 9.47 mmol) in MeCN (20 mL) and H₂O (5 mL) was stirred at 80° C. for 4 hrs. On completion, the mixture was dried by lyophilization to give the title compound (1.4 g) as a light yellow solid. LC-MS (ESI⁺) m/z 154.1 (M+H)⁺.

### 2′-(2-chloropyridin-4-yl)-5′,6′-dihydrospiro[cyclobutane-1,7′-pyrrolo[3,2-c]pyridin]-4′(1′H)-one (Intermediate BL)

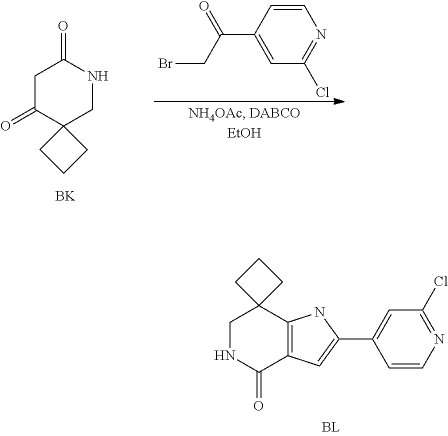

To a solution of 6-azaspiro[3.5]nonane-7,9-dione (600 mg, 3.92 mmol, Intermediate BK) in EtOH (20 mL) was added 2-bromo-1-(2-chloropyridin-4-yl)ethanone (1.38 g, 5.88 mmol, synthesis of Step 1 of Intermediate AC), NH₄OAc (4.53 g, 58.8 mmol) and DABCO (439 mg, 3.92 mmol, 431 uL). The mixture was stirred at 50° C. for 1 hr. On completion, the mixture was quenched by adding H₂O (20 mL) and extracted with DCM (30 mL×3). The organic phase was dried over anhydrous Na₂SO₄, then filtered and concentrated under reduced pressure to give a residue. The residue was purified by column chromatography (SiO₂, Petroleum ether/Ethyl acetate=1/1 to DCM:MeOH=20:1) to give the title compound (620 mg, 52% yield) as a yellow solid. LC-MS (ESI⁺) m/z 288.0 (M+H)⁺.

### 2′-(2-(4-(piperazin-1-yl)phenyl)pyridin-4-yl)-5′,6′-dihydrospiro[cyclobutane-1,7′-pyrrolo[3,2-c]pyridin]-4′(1′H)-one (Intermediate BM)

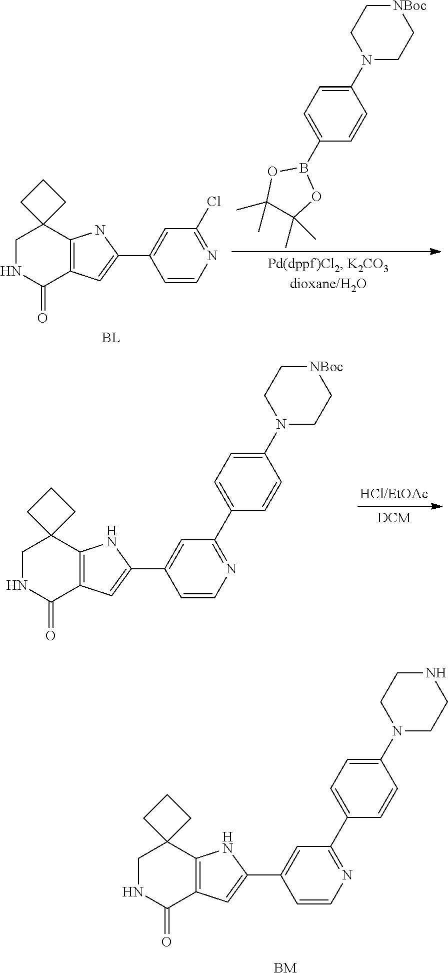

Step 1—Tert-butyl 4-(4-(4-(4′-oxo-1′,4′,5′,6′-tetrahydrospiro[cyclobutane-1,7′-pyrrolo[3,2-c]pyridin]-2′-yl)pyridin-2-yl)phenyl)piperazine-1-carboxylate. To a solution of 2′-(2-chloropyridin-4-yl)-5′,6′-dihydrospiro[cyclobutane-1,7′-pyrrolo[3,2-c]pyridin]-4′(1′H)-one (450 mg, 1.56 mmol, Intermediate BL), tert-butyl 4-(4-(4,4,5,5-tetramethyl-1,3,2-dioxaborolan-2-yl)phenyl)piperazine-1-carboxylate (668 mg, 1.72 mmol), Pd(dppf)Cl₂ (114 mg, 156 umol), and K₂CO₃ (540 mg, 3.91 mmol) and in dioxane (10 mL) and H₂O was degassed and purged with N₂ three times. Then the mixture was stirred at 80° C. for 4 hrs under N₂ atmosphere. On completion, the mixture was concentrated under reduced pressure to give a residue. The crude product was triturated with EtOAc/PE (1:5, 20 mL) at 20° C. for 10 min to give the title compound (1.1 g) as a yellow solid. LC-MS (ESI⁺) m/z 514.2 (M+H)⁺.

Step 2—2′-(2-(4-(Piperazin-1-yl)phenyl)pyridin-4-yl)-5′,6′-dihydrospiro[cyclobutane-1,7′-pyrrolo[3,2-c]pyridin]-4′(1′H)-one. To a solution of tert-butyl 4-(4-(4-(4′-oxo-1′,4′,5′,6′-tetrahydrospiro[cyclobutane-1,7′-pyrrolo[3,2-c]pyridin]-2′-yl)pyridin-2-yl)phenyl)piperazine-1-carboxylate (600 mg, 1.17 mmol) in DCM (5 mL) was added HCl/EtOAc (4 M, 1 mL). The mixture was stirred at 20° C. for 0.5 hr. On completion, the mixture was filtered and concentrated to give the title compound (600 mg, HCl) as a white solid. ¹H NMR (400 MHz, DMSO-d₆) δ ppm 15.97-15.99 (m, 1H), 12.74 (br s, 1H), 9.40 (br s, 2H), 8.78 (s, 1H), 8.52 (br d, J=6.4 Hz, 1H), 8.22 (br d, J=8.8 Hz, 2H), 8.17 (br d, J=6.4 Hz, 1H), 7.66 (s, 1H), 7.40 (br s, 1H), 7.22 (br d, J=8.8 Hz, 2H), 3.65 (br s, 4H), 3.49 (s, 2H), 3.23 (br s, 4H), 2.61-2.68 (m, 2H), 1.98-2.11 (m, 4H). LC-MS (ESI⁺) m/z 414.1 (M+H)⁺.

### Tert-butyl 4-(2-methoxy-5-methyl-4-(4,4,5,5-tetramethyl-1,3,2-dioxaborolan-2-yl)phenyl)piperazine-1-carboxylate (Intermediate BN)

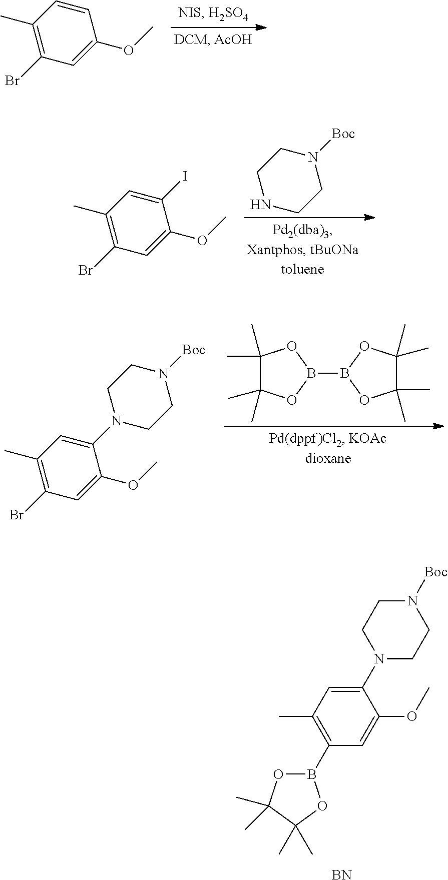

Step 1—1-Bromo-4-iodo-5-methoxy-2-methylbenzene. To a solution of 2-bromo-4-methoxy-1-methylbenzene (5 g, 24.8 mmol, CAS #36942-50-6) in DCM (30 mL) and AcOH (30 mL) was added H₂SO₄ (32.2 g, 328 mmol, 17.5 mL) and 1-iodopyrrolidine-2,5-dione (5.59 g, 24.8 mmol) at 25° C., then the mixture was stirred at 25° C. for 16 hrs. On completion, the reaction mixture was quenched with cold water (100 mL) and extracted by ethyl acetate (50×3 mL). The extracts were washed by brine (50 mL) and dried over anhydrous sodium sulfate, filtered and concentrated in vacuo to give the crude residue. The residue was purified by column chromatography (SiO₂, Petroleum ether/Ethyl acetate=1/0) to give the title compound (8 g, 98% yield) as a red solid. ¹H NMR (400 MHz, DMSO-d₆) δ=7.75 (s, 1H), 7.17 (s, 1H), 3.81 (s, 3H), 2.24 (s, 3H).

Step 2—Tert-butyl 4-(4-bromo-2-methoxy-5-methylphenyl)piperazine-1-carboxylate. To a solution of tert-butyl piperazine-1-carboxylate (570 mg, 3.06 mmol) and 1-bromo-4-iodo-5-methoxy-2-methylbenzene (1 g, 3.06 mmol) in toluene (10 mL) was added Pd₂(dba)₃ (56.0 mg, 61.2 umol), (5-diphenylphosphanyl-9,9-dimethyl-xanthen-4-yl)-diphenyl-phosphane (106 mg, 183 umol) and tBuONa (2 M, 4.59 mL) at 25° C., then the mixture was stirred at 100° C. for 4 hrs. On completion, the reaction mixture was filtered and concentrated in vacuo to give the crude residue. The residue was purified by column chromatography (SiO₂, Petroleum ether/Ethyl acetate=7/1 to 5/1) to give the title compound (870 mg, 65% yield) as a yellow solid. LC-MS (ESI⁺) m/z 387.0. (M+H)⁺.

Step 3—Tert-butyl 4-(2-methoxy-5-methyl-4-(4,4,5,5-tetramethyl-1,3,2-dioxaborolan-2-yl)phenyl)piperazine-1-carboxylate. To a solution of 4,4,4′,4′,5,5,5′,5′-octamethyl-2,2′-bi(1,3,2-dioxaborolane) (527 mg, 2.08 mmol) and tert-butyl 4-(4-bromo-2-methoxy-5-methyl-phenyl)piperazine-1-carboxylate (400 mg, 1.04 mmol) in dioxane (8 mL) was added KOAc (306 mg, 3.11 mmol) and Pd(dppf)Cl₂ (76.0 mg, 104 umol) at 25° C., then the mixture was stirred at 80° C. for 2 hrs. On completion, the reaction filtered and concentrated in vacuo to give the crude residue. The residue was purified by column chromatography (SiO₂, Petroleum ether/Ethyl acetate=7/1 to 5/1) to give the title compound (420 mg, 84% yield) as a yellow oil. LC-MS (ESI⁺) m/z 433.1. (M+H)⁺.

### (R)-tert-butyl 4-(2-methoxy-5-methyl-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (Intermediate BO)

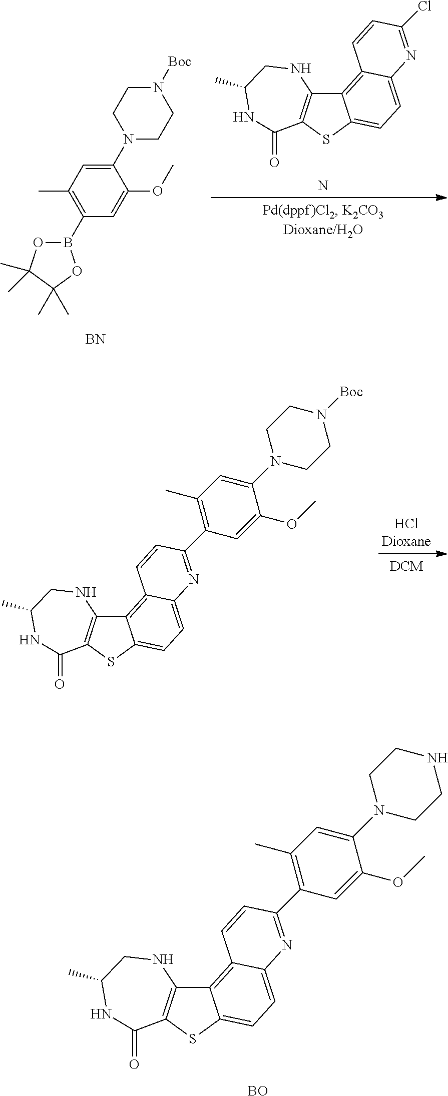

Step 1—(R)-tert-butyl 4-(2-methoxy-5-methyl-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. To a solution of (R)-3-chloro-10-methyl-9,10,11,12-tetrahydro-8H-[1,4]diazepino[5′,6′:4,5]thieno[3,2-f]quinolin-8-one (294 mg, 925 umol, Intermediate N) and tert-butyl 4-(2-methoxy-5-methyl-4-(4,4,5,5-tetramethyl-1,3,2-dioxaborolan-2-yl)phenyl)piperazine-1-carboxylate (400 mg, 925 umol, Intermediate BN) in dioxane (5 mL) and H₂O (0.2 mL) was added K₂CO₃ (383 mg, 2.78 mmol) and Pd(dppf)Cl₂ (67.7 mg, 92.5 umol) at 25° C. Then the mixture was stirred at 80° C. for 2 hrs. On completion, the reaction mixture was filtered and concentrated in vacuo to give the crude residue. The crude product was triturated with MTBE at 25° C. for 30 min to give the title compound (600 mg, 74% yield) as yellow oil. LC-MS (ESI⁺) m/z 588.4. (M+H)⁺.

Step 2—(R)-tert-butyl 4-(2-methoxy-5-methyl-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. To a solution of (R)-tert-butyl 4-(2-methoxy-5-methyl-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (50 mg, 85.1 umol) in DCM (1 mL) was added HCl/dioxane (4 M, 0.2 mL) at 25° C., then the mixture was stirred at 25° C. for 1.5 hrs. On completion, the reaction mixture was filtered and concentrated in vacuo to give the crude residue. The crude product was purified by reversed-phase HPLC (column: Phenomenex luna C18 150×25 mm×10 um; mobile phase: [water (FA)-ACN]; B %: 3%-33%, 15 min) to give the title compound (25 mg, 54% yield, FA) as a yellow solid. ¹H NMR (400 MHz, DMSO-d₆) δ=9.20 (d, J=8.8 Hz, 1H), 8.27 (s, 1H), 8.12 (d, J=9.2 Hz, 1H), 8.08 (br d, J=4.4 Hz, 1H), 7.96 (d, J=8.8 Hz, 1H), 7.80 (d, J=8.8 Hz, 1H), 7.18-7.11 (m, 2H), 6.85 (s, 1H), 3.83 (s, 3H), 3.64-3.58 (m, 1H), 3.49-3.45 (m, 2H), 3.07-2.94 (m, 8H), 2.37 (s, 3H), 1.19 (d, J=6.8 Hz, 3H). LC-MS (ESI⁺) m/z 488.2. (M+H)⁺.

### 3-(3-methyl-2-oxo-5-(2,6-diazaspiro[3.3]heptan-2-yl)-2,3-dihydro-1H-benzo[d]imidazol-1-yl)piperidine-2,6-dione (Intermediate BP)

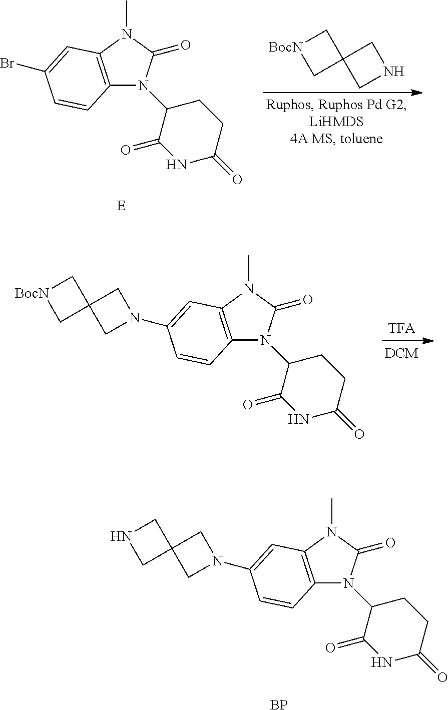 [SMILES: Cn1c(=O)n(C2CCC(=O)NC2=O)c2ccc(Br)cc21 ; CC(C)(C)OC(=O)N1CC2(CNC2)C1 ; Cn1c(=O)n(C2CCC(=O)NC2=O)c2ccc(N3CC4(CN(C(=O)OC(C)(C)C)C4)C3)cc21 ; Cn1c(=O)n(C2CCC(=O)NC2=O)c2ccc(N3CC4(CNC4)C3)cc21 ; "BP"]

Step 1—Tert-butyl 6-(1-(2,6-dioxopiperidin-3-yl)-3-methyl-2-oxo-2,3-dihydro-1H-benzo[d]imidazol-5-yl)-2,6-diazaspiro[3.3]heptane-2-carboxylate. To a solution of 3-(5-bromo-3-methyl-2-oxo-benzimidazol-1-yl)piperidine-2,6-dione (1 g, 2.96 mmol, Intermediate E), 4 Å molecular sieves (1 g) and tert-butyl 2,6-diazaspiro[3.3]heptane-2-carboxylate (879 mg, 4.44 mmol, CAS #1041026-70-3) in toluene (30 mL) was added LiHMDS (1 M, 17.7 mL) at 0° C. and the mixture was purged with N₂ three times. Then RuPhos (69.0 mg, 148 umol) and Ruphos Pd G2 (115 mg, 148 umol) was added and the mixture was stirred at 100° C. for 1 hr. On completion, the reaction mixture was quenched by addition of FA at 0° C. until the pH 6, filtered and concentrated under reduced pressure to give a residue. The residue was purified by column chromatography (SiO₂, Petroleum ether/Ethyl acetate=2/1 to 0/1 and then DCM:EA=1:1) to give the title compound (800 mg, 58% yield) as a yellow solid. LC-MS (ESI+) m/z 455.7 (M+H)⁺.

Step 2—3-(3-Methyl-2-oxo-5-(2,6-diazaspiro[3.3]heptan-2-yl)-2,3-dihydro-1H-benzo[d]imidazol-1-yl)piperidine-2,6-dione. To a solution of tert-butyl 6-[1-(2,6-dioxo-3-piperidyl)-3-methyl-2-oxo-benzimidazol-5-yl]-2,6-diazaspiro[3.3]heptane-2-carboxylate (300 mg, 659 umol) in DCM (5 mL) was added TFA (1.54 g, 13.5 mmol). The mixture was stirred at 25° C. for 1 hr. On completion, the reaction mixture was filtered and concentrated under reduced pressure to give a residue. The residue was purified by reversed-phase HPLC (0.1% FA condition) to give the title compound (230 mg, 84% yield) as a white solid. LC-MS (ESI⁺) m/z 356.1 (M+H)⁺.

### (R)-1-(10-methyl-8-oxo-9,10,11,12-tetrahydro-8H-[1,4]diazepino[5′,6′:4,5]thieno[3,2-f]quinolin-3-yl)piperidine-4-carbaldehyde (Intermediate BQ)

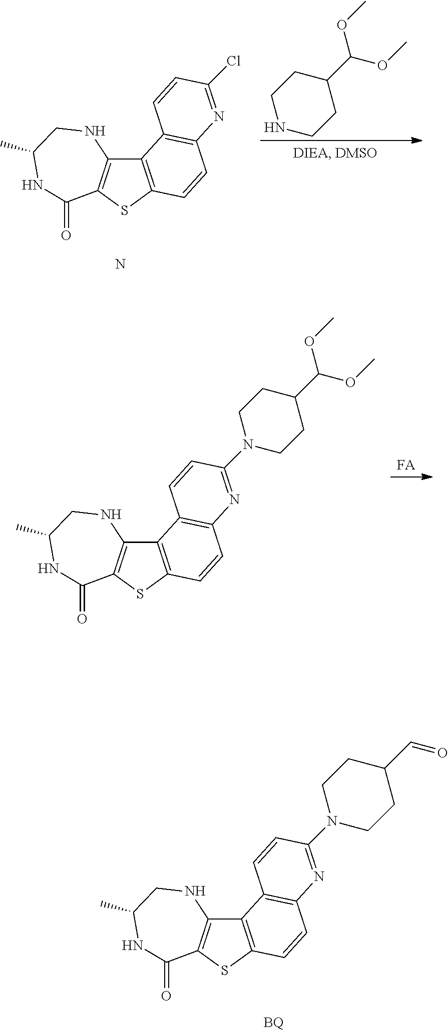

Step 1—(R)-3-(4-(dimethoxymethyl)piperidin-1-yl)-10-methyl-9,10,11,12-tetrahydro-8H-[1,4]diazepino[5′,6′:4,5]thieno[3,2-f]quinolin-8-one. To a solution of (15R)-5-chloro-15-methyl-11-thia-6,14,17-triazatetracyclo[8.8.0.0^{2,7}.0^{12,18}]octadeca-1(10),2(7),3,5,8,12(18)-hexaen-13-one (3 g, 9.44 mmol Intermediate N) and 4-(dimethoxymethyl)piperidine (2.25 g, 14.2 mmol, CAS #188646-83-5) in DMSO (40 mL) was added DIEA (3.66 g, 28.3 mmol). The mixture was stirred at 100° C. for 48 hours. On completion, the reaction mixture was quenched by addition of H₂O (200 mL) at 25° C., and then filtered and concentrated under reduced pressure to give a residue. The residue was purified by column chromatography (SiO₂, Petroleum ether/Ethyl acetate=2/1 to 0/1 and then DCM:EA=1:1) to give the title compound (2.6 g, 54% yield) as a yellow solid. LC-MS (ESI+) m/z 441.1 (M+H)⁺.

Step 2—(R)-1-(10-methyl-8-oxo-9,10,11,12-tetrahydro-8H-[1,4]diazepino[5′,6′:4,5]thieno[3,2-f]quinolin-3-yl)piperidine-4-carbaldehyde. (15R)-5-[4-(dimethoxymethyl)-1-piperidyl]-15-methyl-1l-thia-6,14,17-triazatetracyclo[8.8.0.0^{2,7}.0^{12,18}]octadeca-1(10),2(7),3,5,8,12(18)-hexaen-13-one (80 mg, 182 umol) in HCOOH (1 mL) was stirred at 50° C. for 1 hr. On completion, the reaction mixture was filtered and concentrated under reduced pressure to give the title compound (80 mg, FA) as a yellow solid. LC-MS (ESI+) m/z 413.1 (M+H₂O).

### Tert-butyl 4-(3-fluoro-4-(4,4,5,5-tetramethyl-1,3,2-dioxaborolan-2-yl)phenyl)piperazine-1-carboxylate (Intermediate BR)

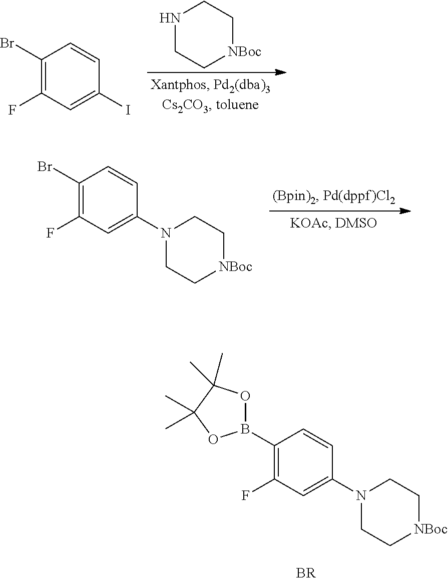 [SMILES: Fc1cc(I)ccc1Br ; CC(C)(C)OC(=O)N1CCNCC1 ; CC(C)(C)OC(=O)N1CCN(c2ccc(Br)c(F)c2)CC1 ; CC(C)(C)OC(=O)N1CCN(c2ccc(B3OC(C)(C)C(C)(C)O3)c(F)c2)CC1]

Step 1—Tert-butyl 4-(4-bromo-3-fluorophenyl)piperazine-1-carboxylate. To a solution of 1-bromo-2-fluoro-4-iodobenzene (20.2 g, 67.1 mmol, CAS #1146950-53-9) and tert-butyl piperazine-1-carboxylate (5 g, 26.9 mmol, CAS #143238-38-4) in toluene (100 mL) was added (5-diphenylphosphanyl-9,9-dimethyl-xanthen-4-yl)-diphenyl-phosphane (932 mg, 1.61 mmol), Cs₂CO₃ (34.9 g, 107 mmol) and Pd₂(dba)₃ (492 mg, 537 umol) at 25° C., then the mixture was stirred at 100° C. for 4 hrs. On completion, the reaction mixture was filtered and concentrated in vacuo to give the crude residue. The residue was purified by column chromatography (SiO₂, Petroleum ether/Ethyl acetate=70/1 to 10/1) to give the title compound (5.8 g, 55% yield) as white solid. LC-MS (ESI+) m/z 304.7. (M+H)⁺.

Step 2—Tert-butyl 4-(3-fluoro-4-(4,4,5,5-tetramethyl-1,3,2-dioxaborolan-2-yl)phenyl)piperazine-1-carboxylate. To a solution of tert-butyl 4-(4-bromo-3-fluorophenyl)piperazine-1-carboxylate (1 g, 2.78 mmol) and 4,4,5,5-tetramethyl-2-(4,4,5,5-tetramethyl-1,3,2-dioxaborolan-2-yl)-1,3,2-dioxaborolane (2.12 g, 8.35 mmol) in DMSO (10 mL) was added AcOK (819 mg, 8.35 mmol) and Pd(dppf)Cl₂ (407 mg, 556 umol) at 25° C., then the mixture was stirred at 100° C. for 2 hrs. On completion, the reaction mixture was quenched with water (20 mL) and extracted with dichloromethane (3×20 mL). The extracts were washed with brine (60 mL) and dried over anhydrous sodium sulfate, filtered and concentrated in vacuo to get the crude residue. The residue was purified by column chromatography (SiO₂, Petroleum ether/Ethyl acetate=20/1 to 10/1) to give the title compound (900 mg, 70% yield) as white solid. LC-MS (ESI⁺) m/z 407.0. (M+H)⁺.

### 2-(2-(2-Fluoro-4-(piperazin-1-yl)phenyl)pyridin-4-yl)-6,7-dihydro-1H-pyrrolo[3,2-c]pyridin-4(5H)-one (Intermediate BS)

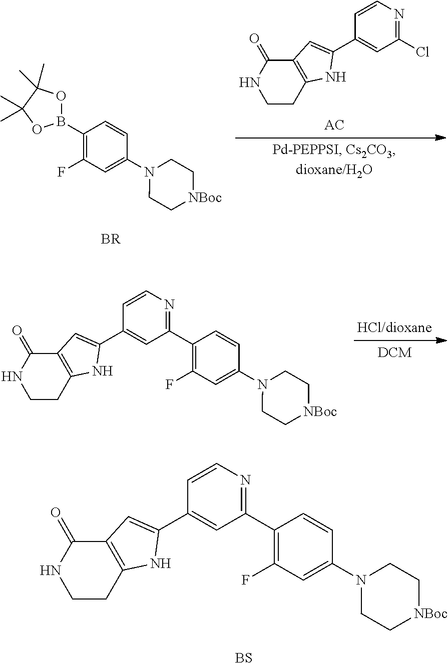 [SMILES: CC(C)(C)OC(=O)N1CCN(c2ccc(B3OC(C)(C)C(C)(C)O3)c(F)c2)CC1 ; O=C1NCCc2[nH]c(-c3ccnc(Cl)c3)cc21 ; CC(C)(C)OC(=O)N1CCN(c2ccc(-c3cc(-c4cc5c([nH]4)CCNC5=O)ccn3)c(F)c2)CC1 ; CC(C)(C)OC(=O)N1CCN(c2ccc(-c3cc(-c4cc5c([nH]4)CCNC5=O)ccn3)c(F)c2)CC1 ; "BS"]

Step 1—Tert-butyl 4-(3-fluoro-4-(4-(4-oxo-4,5,6,7-tetrahydro-1H-pyrrolo[3,2-c]pyridin-2-yl)pyridin-2-yl)phenyl)piperazine-1-carboxylate. To a solution of tert-butyl 4-(3-fluoro-4-(4,4,5,5-tetramethyl-1,3,2-dioxaborolan-2-yl)phenyl)piperazine-1-carboxylate (533.13 mg, 1.31 mmol, Intermediate BR) and 2-(2-chloropyridin-4-yl)-6,7-dihydro-1H-pyrrolo[3,2-c]pyridin-4(5H)-one (250 mg, 1.01 mmol, Intermediate AC) in dioxane (7.5 mL) and H₂O (1.5 mL) was added Pd-PEPPSI-IHeptCl (98.2 mg, 101 umol) and Cs₂CO₃ (2 M, 986 mg) at 25° C. Then the mixture was stirred at 80° C. for 2 hrs. On completion, the reaction mixture was filtered and concentrated in vacuo to get the crude residue. The residue was purified by column chromatography (SiO₂, Petroleum ether/Ethyl acetate=12/1 to 8/1) to give the title compound (480 mg, 92% yield) as colorless oil. LC-MS (ESI⁺) m/z 492.3. (M+H)⁺.

Step 2—2-(2-(2-Fluoro-4-(piperazin-1-yl)phenyl)pyridin-4-yl)-6,7-dihydro-1H-pyrrolo[3,2-c]pyridin-4(5H)-one. To a solution of tert-butyl 4-(3-fluoro-4-(4-(4-oxo-4,5,6,7-tetrahydro-1H-pyrrolo[3,2-c]pyridin-2-yl)pyridin-2-yl)phenyl)piperazine-1-carboxylate (150 mg, 305 umol) in DCM (1 mL) was added HCl/dioxane (4 M, 76.3 uL) at 25° C., then the mixture was stirred at 25° C. for 1 hr. On completion, the reaction mixture was filtered and concentrated in vacuo to give the title compound (150 mg) as yellow solid. LC-MS (ESI⁺) m/z 392.1.

### Tert-butyl 4-(5-fluoro-2-methoxy-4-(4,4,5,5-tetramethyl-1,3,2-dioxaborolan-2-yl)phenyl)piperazine-1-carboxylate (Intermediate BT)

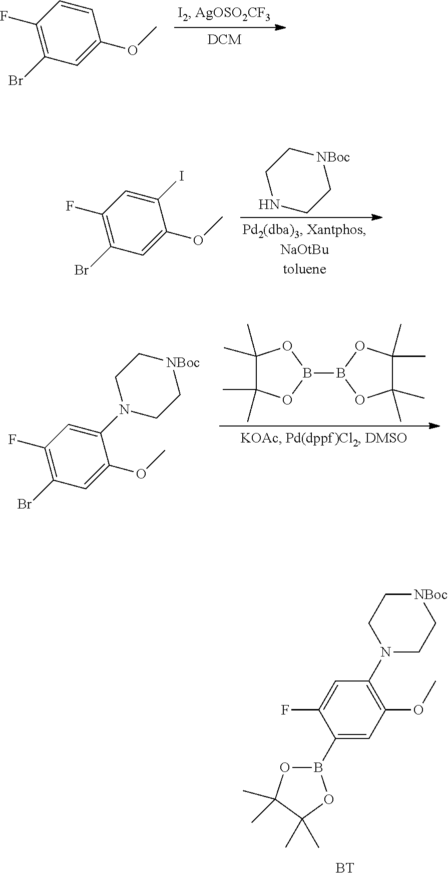

Step 1—1-Bromo-2-fluoro-4-iodo-5-methoxybenzene. A solution of 2-bromo-1-fluoro-4-methoxybenzene (4 g, 19.51 mmol, CAS #1161497-23-9) and silver trifluoromethanesulfonate (5.51 g, 21.5 mmol) in DCM (40 mL) was stirred at 25° C. for 0.3 hr. Then I₂ (5.45 g, 21.5 mmol, 4.32 mL) was added at 25° C. and the mixture was stirred at 25° C. for 16 hrs. On completion, the mixture was diluted with DCM (40 mL) and filtered through celite. The celite bed was washed with DCM (2×20 ml) and the filtrate was washed with 20% aqueous sodium thiosulfate (40 ml) and saturated aqueous sodium bicarbonate solution (40 ml). The organic layer was dried over sodium sulfate, filtered and the filtrate was concentrated under reduced pressure. The residue was purified by column chromatography (SiO₂, Petroleum ether/Ethyl acetate=1/0 to 10/1) to give the title compound (3.1 g, 48% yield) as a white solid.

Step 2—Tert-butyl 4-(4-bromo-5-fluoro-2-methoxyphenyl)piperazine-1-carboxylate. To a solution of 1-bromo-2-fluoro-4-iodo-5-methoxybenzene (1.8 g, 5.44 mmol) and tert-butyl piperazine-1-carboxylate (1.01 g, 5.44 mmol) in toluene (26 mL) was added sodium; 2-methylpropan-2-olate (1.57 g, 16.3 mmol), Xantphos (315 mg, 544 umol) and (1E, 4E)-1, 5-diphenylpenta-1, 4-dien-3-one;palladium (199 mg, 218 umol). The mixture was stirred at 60° C. for 3 hrs. On completion, the reaction mixture was concentrated under reduced pressure to give a residue. The residue was purified by column chromatography (SiO₂, Petroleum ether/Ethyl acetate=1/0 to 5/1) to give the title compound (1.42 g, 58% yield) as a white solid. LC-MS (ESI+) m/z 391.0 (M+H)⁺.

Step 3—Tert-butyl 4-(5-fluoro-2-methoxy-4-(4,4,5,5-tetramethyl-1,3,2-dioxaborolan-2-yl)phenyl)piperazine-1-carboxylate. A solution of tert-butyl 4-(4-bromo-5-fluoro-2-methoxyphenyl)piperazine-1-carboxylate (598 mg, 1.54 mmol), 4,4,4′,4′,5,5,5′,5′-octamethyl-2,2′-bi(1,3,2-dioxaborolane) (1.17 g, 4.61 mmol), KOAc (453 mg, 4.61 mmol) and cyclopentyl(diphenyl)phosphane; dichloromethane; dichloropalladium; iron (125.55 mg, 153.74 umol) were taken up into a microwave tube in DMSO (10 mL). The sealed tube was heated at 100° C. for 120 min under microwave. On completion, the reaction mixture was partitioned between EA (3 mL) and H₂O (3 mL). The organic phase was separated, washed with brine (2 mL×3), dried over Na₂SO₄, filtered and the filtrate was concentrated under reduced pressure to give a residue. The residue was purified by column chromatography (SiO₂, Petroleum ether/Ethyl acetate=1/0 to 1/1) to give the title COMPOUND (543 mg, 59% yield) as a white solid. LC-MS (ESI⁺) m/z 437.6 (M+H)⁺.

### (R)-3-(2-fluoro-5-methoxy-4-(piperazin-1-yl)phenyl)-10-methyl-9,10,11,12-tetrahydro-8H-[1,4]diazepino[5′,6′:4,5]thieno[3,2-f]quinolin-8-one (Intermediate BU)

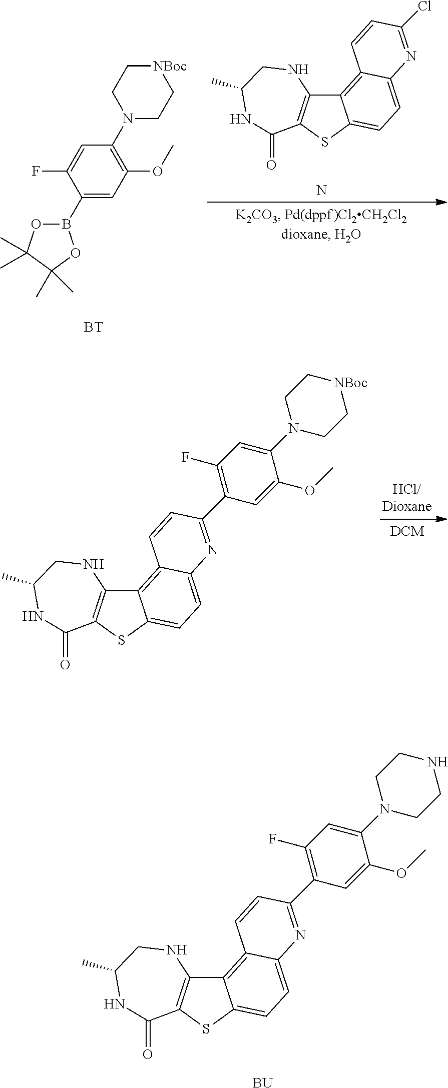

Step 1—(R)-tert-butyl 4-(5-fluoro-2-methoxy-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. A mixture of tert-butyl 4-(5-fluoro-2-methoxy-4-(4,4,5,5-tetramethyl-1,3,2-dioxaborolan-2-yl) phenyl)piperazine-1-carboxylate (400 mg, 917 umol, Intermediate BT), (R)-3-chloro-10-methyl-9,10,11,12-tetrahydro-8H-[1,4]diazepino[5′,6′:4,5]thieno[3,2-f]quinolin-8-one (291 mg, 917 umol, Intermediate N), K₂CO₃ (380 mg, 2.75 mmol), and Pd(dppf)Cl₂.CH₂Cl₂ (37.4 mg, 45.8 umol) in dioxane (4 mL) and H₂O (2 mL) was degassed and purged with N₂ three times. Then the mixture was stirred at 80° C. for 2 hrs under N₂ atmosphere. On completion, the reaction mixture was concentrated under reduced pressure to remove solvent. The residue was purified by column chromatography (SiO₂, Petroleum ether/Ethyl acetate=1/0 to 1/1) to give the title COMPOUND (275 mg, 37% yield) as a yellow solid. LC-MS (ESI⁺) m/z 592.1 (M+H)⁺.

Step 2—(R)-3-(2-fluoro-5-methoxy-4-(piperazin-1-yl)phenyl)-10-methyl-9,10,11,12-tetrahydro-8H-[1,4]diazepino[5′,6′:4,5]thieno[3,2-f]quinolin-8-one. To a solution of (R)-tert-butyl 4-(5-fluoro-2-methoxy-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (50 mg, 84.5 umol) in DCM (1 mL) was added HCl/Dioxane (4 M, 42.2 uL). The mixture was stirred at 25° C. for 0.5 hr. On completion, the reaction mixture was concentrated under reduced pressure to remove solvent. The residue was purified by prep-HPLC (column: Welch Ultimate AQ-C18 150×30 mm×5 um; mobile phase: [water (HCl)-ACN]; B %: 4%-34%, 10 min) to give title COMPOUND (9.5 mg, 21% yield, HCl) as a red solid. ¹H NMR (400 MHz, DMSO-d₆) δ=9.28 (d, J=8.8 Hz, 1H), 9.12 (s, 1H), 8.16 (d, J=8.8 Hz, 1H), 8.12 (d, J=4.0 Hz, 1H), 8.08-8.02 (m, 2H), 7.76 (d, J=7.6 Hz, 1H), 7.00 (d, J=13.2 Hz, 1H), 3.92 (s, 3H), 3.60 (d, J=3.6 Hz, 1H), 3.52-3.44 (m, 2H), 3.38-3.32 (m, 4H), 3.28 (s, 4H), 1.20 (d, J=6.8 Hz, 3H). LC-MS (ESI⁺) m/z 492.1 (M+H)⁺.

### (R)-10-methyl-3-(2-oxo-7-azaspiro[3.5]nonan-7-yl)-9,10,11,12-tetrahydro-8H-[1,4]diazepino[5′,6′:4,5]thieno[3,2-f]quinolin-8-one (Intermediate BV)

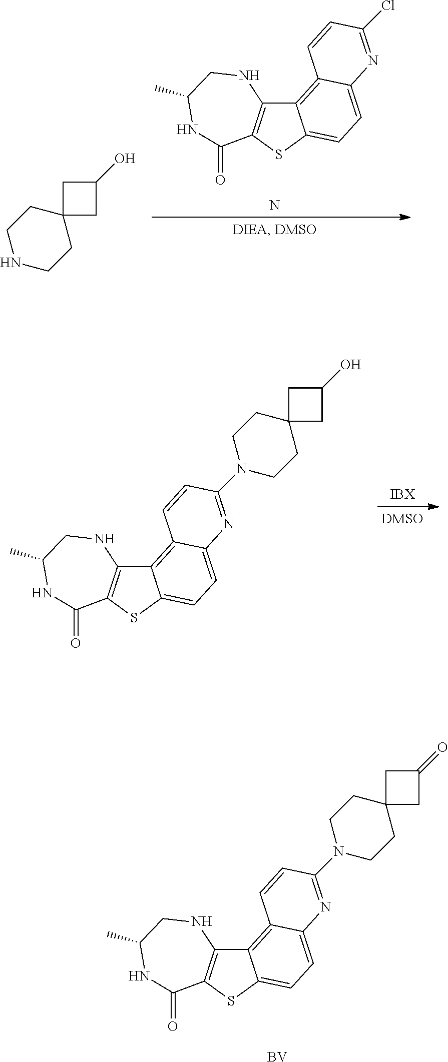

Step 1—(R)-3-(2-hydroxy-7-azaspiro[3.5]nonan-7-yl)-10-methyl-9,10,11,12-tetrahydro-8H-[1,4]diazepino[5′,6′:4,5]thieno[3,2-f]quinolin-8-one. To a solution of 7-azaspiro[3.5]nonan-2-ol (895 mg, 6.33 mmol, CAS #784137-09-3) in DMSO (10 mL) was added (15R)-5-chloro-15-methyl-11-thia-6,14,17-triazatetracyclo[8.8.0.0^{2,7}.0^{12,18}]octadeca-1,3,5,7,9,12(18)-hexaen-13-one (800 mg, 2.52 mmol, Intermediate N) and DIEA (1.63 g, 12.6 mmol, 2.19 mL). The mixture was stirred at 130° C. for 12 hrs. On completion, the reaction mixture was quenched with H₂O (20 mL) at 25° C., and then extracted with EA (100 mL×3). The combined organic layers were washed with NaCl (50 mL×2), dried over Na₂SO₄, filtered and concentrated under reduced pressure to give a residue. The residue was purified by column chromatography (SiO₂, DCM:MeOH=30:1) to give a title compound (580 mg, 50% yield) as a brown solid. LC-MS (ESI⁺) m/z 423.2 (M+H)⁺.

Step 2—(R)-10-methyl-3-(2-oxo-7-azaspiro[3.5]nonan-7-yl)-9,10,11,12-tetrahydro-8H-[1,4]diazepino[5′,6′:4,5]thieno[3,2-f]quinolin-8-one. To a solution of (15R)-5-(2-hydroxy-7-azaspiro[3.5]nonan-7-yl)-15-methyl-11-thia-6,14,17-triazatetracyclo[8.8.0.0^{2,7}.0^{12,18}]octadeca-1,3,5,7,9,12(18)-hexaen-13-one (450 mg, 1.06 mmol) in DMSO (5 mL) was added IBX (596 mg, 2.13 mmol). The mixture was stirred at 25° C. for 1 hr. On completion, the reaction mixture was concentrated under reduced pressure to give a residue. The residue was purified by prep-HPLC (FA condition) to give a title compound (50 mg, 9% yield) as a white solid. LC-MS (ESI⁺) m/z 421.0. (M+H)⁺.

### 3-(3-methyl-2-oxo-4-piperazin-1-yl-benzimidazol-1-yl)piperidine-2,6-dione (Intermediate BW)

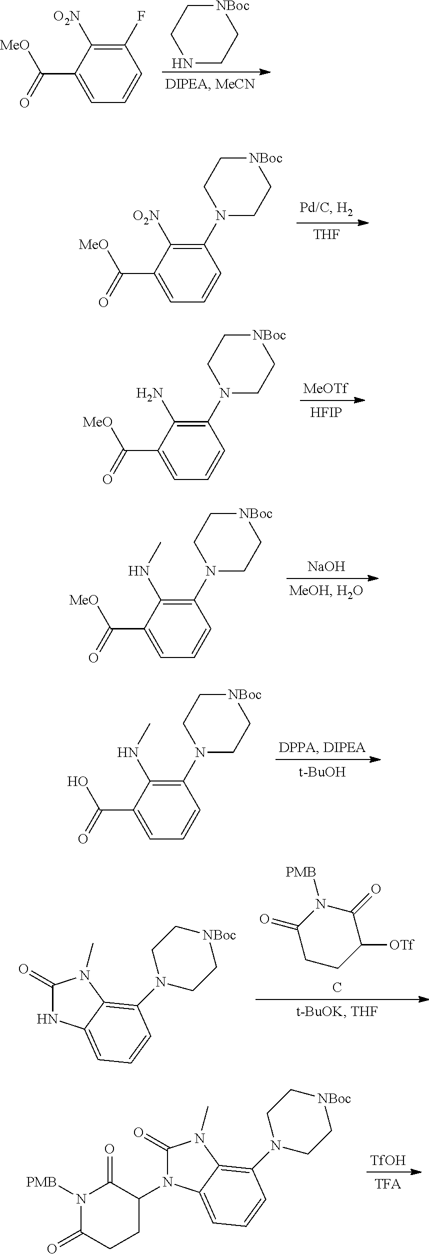

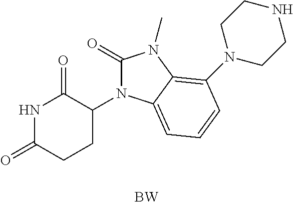 [SMILES: Cn1c(=O)n(C2CCC(=O)NC2=O)c2cccc(N3CCNCC3)c21 ; "BW"]

Step 1—Tert-butyl 4-(3-methoxycarbonyl-2-nitro-phenyl)piperazine-1-carboxylate. To a solution of methyl 3-fluoro-2-nitro-benzoate (10.0 g, 50.2 mmol, CAS #1214353-57-7) and tert-butyl piperazine-1-carboxylate (11.2 g, 60.3 mmol, CAS #143238-38-4) in ACN (100 mL) was added DIPEA (19.5 g, 151 mmol). The reaction mixture was stirred at 50° C. for 12 hrs. On completion, the mixture was concentrated in vacuo. The residue was dissolved in water (200 mL), then extracted with EA (2×200 mL). The organic layer was washed with brine (2×100 mL), dried with Na₂SO₄, filtered and the filtrate was concentrated in vacuo to give the title compound (18.3 g, 100% yield) as yellow oil. ¹H NMR (400 MHz, DMSO-d₆) δ 7.86 (dd, J=1.2, 8.0 Hz, 1H), 7.82-7.79 (m, 1H), 7.74-7.68 (m, 1H), 3.83 (s, 3H), 3.40-3.35 (m, 4H), 2.88-2.84 (m, 4H), 1.41 (s, 9H).

Step 2—Tert-butyl 4-(2-amino-3-methoxycarbonyl-phenyl)piperazine-1-carboxylate. To a solution of tert-butyl 4-(3-methoxycarbonyl-2-nitro-phenyl)piperazine-1-carboxylate (17.0 g, 46.5 mmol) in THF (15 mL) was added Pd/C (2.00 g, 10 wt %). The reaction mixture was stirred at 20° C. for 12 hrs under H₂ (15 Psi) atmosphere. On completion, the mixture was filtered and the filtrate was concentrated in vacuo to give the title compound (15.2 g, 97% yield) as a yellow solid. ¹H NMR (400 MHz, CDCl₃) δ 7.67 (dd, J=1.2, 8.0 Hz, 1H), 7.10 (dd, J=1.2, 7.6 Hz, 1H), 6.61 (t, J=7.6 Hz, 1H), 6.24 (br s, 2H), 4.28-3.95 (m, 2H), 3.87 (s, 3H), 3.16-2.84 (m, 4H), 2.80-2.55 (m, 2H), 1.49 (s, 9H).

Step 3—Tert-butyl 4-[3-methoxycarbonyl-2-(methylamino)phenyl]piperazine-1-carboxylate. To a solution of tert-butyl 4-(2-amino-3-methoxycarbonyl-phenyl)piperazine-1-carboxylate (15.0 g, 44.7 mmol) in 1,1,1,3,3,3-hexafluoropropan-2-ol (40 mL) was added methyl trifluoromethanesulfonate (9.54 g, 58.1 mmol) at 0° C. The reaction mixture was stirred at 0° C. for 0.5 hr. On completion, the mixture was diluted with water (200 mL), then extracted with EA (2×200 mL). The organic layer was washed with brine (2×200 mL), dried with Na₂SO₄, filtered and the filtrate was concentrated in vacuo to give the title compound (15.0 g, 96% yield) as a yellow solid. ¹H NMR (400 MHz, DMSO-d₆) δ 8.16-7.90 (m, 1H), 7.46 (dd, J=1.2, 8.0 Hz, 1H), 7.23 (d, J=7.6 Hz, 1H), 6.77 (t, J=7.6 Hz, 1H), 3.80 (s, 3H), 3.55-3.45 (m, 4H), 2.87 (s, 3H), 2.80-2.74 (m, 4H), 1.42 (s, 9H).

Step 4—3-(4-Tert-butoxycarbonylpiperazin-1-yl)-2-(methylamino)benzoic acid. To a solution of tert-butyl 4-[3-methoxycarbonyl-2-(methylamino)phenyl]piperazine-1-carboxylate (14.0 g, 40.1 mmol) in a mixed solvent of H₂O (20 mL) and MeOH (140 mL) was added NaOH (4.81 g, 120 mmol). The reaction mixture was stirred at 70° C. for 12 hrs. On completion, the mixture was concentrated in vacuo. The residue was diluted with water (200 mL), and extracted with EA (100 mL). The organic layer was discarded. The aqueous phase was acidified with HCl (1N) to pH=3-5, and extracted with EA (2×100 mL). The organic layer was washed with brine (200 mL), dried with Na₂SO₄, filtered and the filtrate was concentrated in vacuo. The residue was triturated with MeOH/H₂O (1:10, 100 mL) and filtered. The filter cake was dried in vacuo to give the title compound (9.60 g, 71% yield) as a white solid. LC-MS (ESI⁺) m/z 336.1 (M+H)⁺.

Step 5—Tert-butyl 4-(3-methyl-2-oxo-1H-benzimidazol-4-yl)piperazine-1-carboxylate. To a solution of 3-(4-tert-butoxycarbonylpiperazin-1-yl)-2-(methylamino)benzoic acid (9.60 g, 28.6 mmol) and DIPEA (11.1 g, 85.9 mmol) in t-BuOH (200 mL) was added DPPA (7.88 g, 28.6 mmol). The reaction mixture was stirred at 85° C. for 12 hrs. On completion, the mixture was concentrated in vacuo. The residue was diluted with water (200 mL), and extracted with EA (2×200 mL). The organic layer was washed with brine (200 mL) and concentrated in vacuo. The residue was purified by reverse phase (0.1% FA condition) to give the title compound (3.35 g, 35% yield) as a white solid. ¹H NMR (400 MHz, DMSO-d₆) δ 10.84 (s, 1H), 6.94-6.87 (m, 1H), 6.85-6.79 (m, 1H), 6.75 (dd, J=1.2, 7.6 Hz, 1H), 4.06-3.80 (m, 2H), 3.55 (s, 3H), 3.20-2.87 (m, 4H), 2.76-2.56 (m, 2H), 1.42 (s, 9H).

Step 6—Tert-butyl 4-[1-[1-[(4-methoxyphenyl)methyl]-2,6-dioxo-3-piperidyl]-3-methyl-2-oxo-benzimidazol-4-yl]piperazine-1-carboxylate. To a solution of tert-butyl 4-(3-methyl-2-oxo-1H-benzimidazol-4-yl)piperazine-1-carboxylate (3.30 g, 9.93 mmol) in THF (50 mL) was added t-BuOK (1.67 g, 14.9 mmol) at 0° C. 1 hr later, and a solution of [1-[(4-methoxyphenyl)methyl]-2,6-dioxo-3-piperidyl] trifluoromethanesulfonate (4.54 g, 11.9 mmol, Intermediate C) in THF (20 mL) was added. The reaction mixture was stirred at 0° C. for 3 hrs. On completion, the mixture was acidified with FA to pH=3-5, diluted with water (300 mL), then extracted with EA (2×300 mL). The organic layer was washed with brine (200 mL) and concentrated in vacuo. The residue was purified by reverse phase flash (0.1% FA condition) to give the title compound (3.90 g, 70% yield) as a white solid. LC-MS (ESI⁺) m/z 564.3 (M+H)⁺.

Step 7-3-(3-Methyl-2-oxo-4-piperazin-1-yl-benzimidazol-1-yl)piperidine-2,6-dione. To a solution of tert-butyl 4-[1-[1-[(4-methoxyphenyl)methyl]-2,6-dioxo-3-piperidyl]-3-methyl-2-oxo-benzimidazol-4-yl]piperazine-1-carboxylate (3.90 g, 6.92 mmol) in TFA (40 mL) was added TfOH (5 mL). The reaction mixture was stirred at 65° C. for 12 hrs. On completion, the mixture was concentrated in vacuo. The residue was purified by reverse phase flash (0.1% FA condition) to give the title compound (1.70 g, 63% yield, FA salt) as a blue solid. ¹H NMR (400 MHz, DMSO-d₆) δ 11.09 (s, 1H), 7.06-6.99 (m, 1H), 6.96-6.92 (m, 2H), 5.36 (dd, J=5.2, 12.4 Hz, 1H), 3.63 (s, 3H), 3.35-3.25 (m, 4H), 3.16-2.97 (m, 4H), 2.91-2.82 (m, 1H), 2.76-2.57 (m, 2H), 2.05-1.93 (m, 1H).

### 4-(4-(2,6-Dioxopiperidin-3-yl)phenoxy)butanal (Intermediate BX)

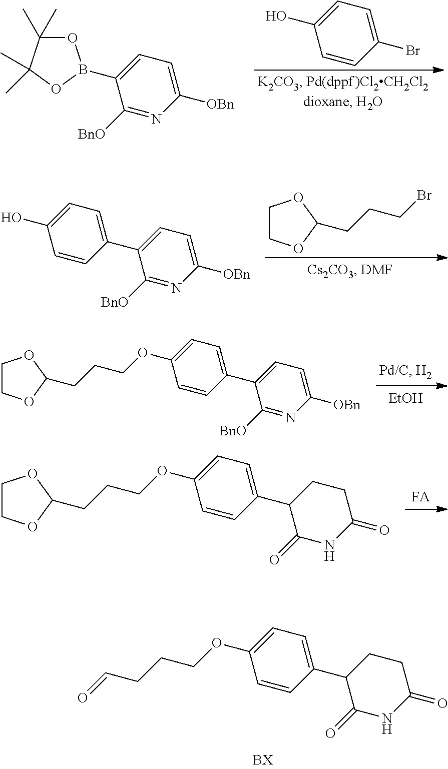

Step 1—4-(2,6-bis(benzyloxy)pyridin-3-yl)phenol. A mixture of 2,6-dibenzyloxy-3-(4,4,5,5-tetramethyl-1,3,2-dioxaborolan-2-yl)pyridine (9.5 g, 22.8 mmol, CAS #2152673-80-6), 4-bromophenol (3.94 g, 22.77 mmol), K₂CO₃ (9.44 g, 68.30 mmol), Pd(dppf)Cl₂.CH₂Cl₂ (1.86 g, 2.28 mmol, CAS #106-41-2) in dioxane (150 mL) and H₂O (30 mL) was degassed and purged with N₂ three times. Then the mixture was stirred at 80° C. for 12 hrs under N₂ atmosphere. On completion, the reaction mixture was diluted with H₂O (200 mL) and extracted with EA (200 mL×2). The combined organic layers were washed with brine (200 mL×2), dried over Na₂SO₄, filtered and the filtrate was concentrated under reduced pressure to give a residue. The residue was purified by column chromatography (SiO₂, Petroleum ether/Ethyl acetate=10/1 to 5/1) to give the title compound (7 g, 79% yield) as an off-white solid. LC-MS (ESI⁺) m/z 384.2. (M+H)⁺.

Step 2—3-(4-(3-(1,3-Dioxolan-2-yl)propoxy)phenyl)-2,6-bis(benzyloxy)pyridine. To a solution of 4-(2,6-dibenzyloxy-3-pyridyl)phenol (300 mg, 782 umol) in DMF (10 mL) was added 2-(3-bromopropyl)-1,3-dioxolane (229 mg, 1.17 mmol, CAS #62563-07-9) and Cs₂CO₃ (765 mg, 2.35 mmol). The reaction was stirred at 65° C. for 12 hrs. On completion, the reaction mixture was concentrated under reduced pressure to give a residue. The residue was purified by column chromatography (SiO₂, Petroleum ether/Ethyl acetate=1/0 to 10/1) to give the title compound (320 mg, 74% yield) as a colorless liquid. LC-MS (ESI⁺) m/z 498.4. (M+H)⁺.

Step 3—3-(4-(3-(1,3-Dioxolan-2-yl)propoxy)phenyl)piperidine-2,6-dione. To a solution of 2,6-dibenzyloxy-3-[4-[3-(1,3-dioxolan-2-yl)propoxy]phenyl]pyridine (320 mg, 643 umol) in EtOH (5 mL) was added Pd/C (160 mg, 10 wt %) under N₂ atmosphere. The suspension was degassed and purged with H₂ three times. The mixture was stirred under H₂ (15 psi) at 20° C. for 2 hrs. On completion, the reaction solution was filtered, then the filtrate was extracted with DCM (40 mL×3). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=10/1 to 2/1) to give the title compound (30 mg, 15% yield) as a white solid. 1H NMR (400 M Hz, DMSO-d6) δ ppm 10.80 (s, 1H), 7.12 (d, J=8.8 Hz, 2H), 6.88 (d, J=8.4 Hz, 2H), 4.85 (t, J=4.4 Hz, 1H), 3.98 (t, J=6.4 Hz, 2H), 3.86-3.91 (m, 2H), 3.75-3.81 (m, 3H), 2.64 (td, J=11.6, 5.6 Hz, 1H), 2.43-2.49 (m, 1H), 2.10-2.22 (m, 1H), 1.97-2.05 (m, 1H), 1.68-1.81 (m, 4H).

Step 4—4-(4-(2,6-Dioxopiperidin-3-yl)phenoxy)butanal. 3-[4-[3-(1,3-Dioxolan-2-yl)propoxy]phenyl]piperidine-2,6-dione (30 mg, 93.9 umol) was added to HCOOH (2 mL) and the reaction was stirred at 25° C. for 1 hr. On completion, the reaction mixture was concentrated under reduced pressure to give the title compound (30 mg) as a brown solid. LC-MS (ESI⁺) m/z 275.8. (M+H)⁺.

### 1-(4-(1-(2,6-dioxopiperidin-3-yl)-3-methyl-2-oxo-2,3-dihydro-1H-benzo[d]imidazol-5-yl)phenyl)piperidine-4-carbaldehyde (Intermediate BY)

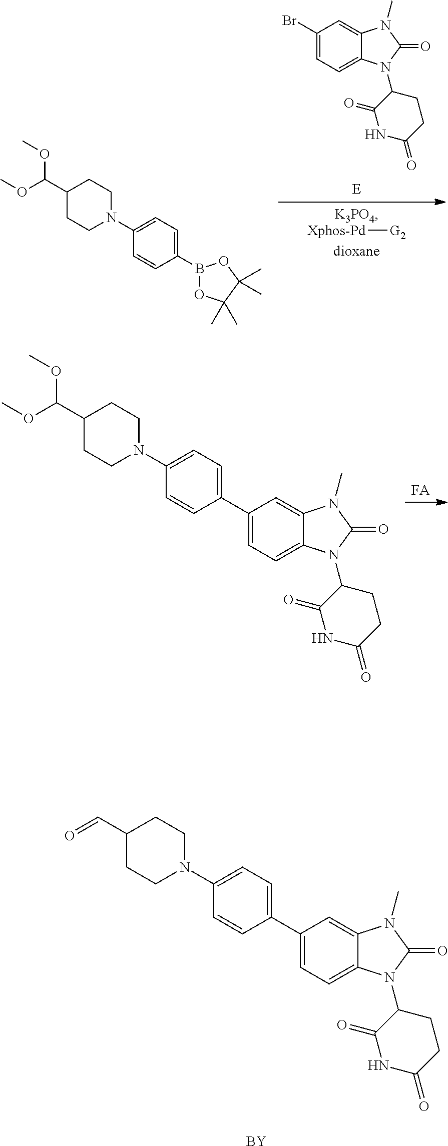

Step 1—3-(5-(4-(4-(Dimethoxymethyl)piperidin-1-yl)phenyl)-3-methyl-2-oxo-2,3-dihydro-1H-benzo[d]imidazol-1-yl)piperidine-2,6-dione. A mixture of 4-(dimethoxymethyl)-1-[4-(4,4,5,5-tetramethyl-1,3,2-dioxaborolan-2-yl)phenyl]piperidine (200 mg, 553 umol, CAS #2242744-56-3), 3-(5-bromo-3-methyl-2-oxo-benzimidazol-1-yl)piperidine-2,6-dione (144 mg, 425 umol, Intermediate E), K₃PO₄ (271 mg, 1.28 mmol,), and XPhos-PD-G2 (33.5 mg, 42.8 umol) in dioxane (4 mL) and H₂O (0.8 mL) was degassed and purged with N₂ three times, and then the mixture was stirred at 60° C. for 12 hrs under N₂ atmosphere. On completion, the reaction mixture was diluted with H₂O (10 mL) and extracted with EA (10 mL×3). The combined organic layers were washed with brine (10 mL×3), dried over Na₂SO₄, filtered and concentrated under reduced pressure to give a residue. The crude product was triturated with EA at 25° C. for 10 min to give the title compound (160 mg, 52% yield) as a white solid. LC-MS (ESI⁺) m/z 493.1 (M+H)⁺.

Step 2—1-(4-(1-(2,6-Dioxopiperidin-3-yl)-3-methyl-2-oxo-2,3-dihydro-1H-benzo[d]imidazol-5-yl)phenyl)piperidine-4-carbaldehyde. A solution of 3-(5-(4-(4-(dimethoxymethyl)piperidin-1-yl)phenyl)-3-methyl-2-oxo-2,3-dihydro-1H-benzo[d]imidazol-1-yl)piperidine-2,6-dione (80 mg, 162 umol) in FA (1.5 mL) was stirred at 40° C. for 1 hr. On completion, the reaction mixture was concentrated under reduced pressure to give the title compound (84 mg, 53% yield) as a brownish black solid. LC-MS (ESI⁺) m/z 465.1 (M+H)⁺.

### Tert-butyl 4-(3-(4,4,5,5-tetramethyl-1,3,2-dioxaborolan-2-yl)-5-((trimethylsilyl)ethynyl)phenyl)piperazine-1-carboxylate (Intermediate BZ)

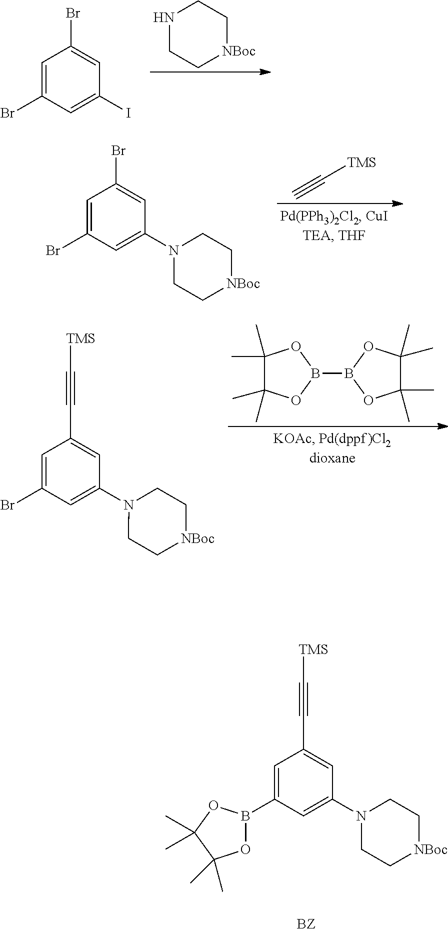

Step 1—Tert-butyl 4-(3,5-dibromophenyl)piperazine-1-carboxylate. To a solution of 1,3-dibromo-5-iodo-benzene (25 g, 69.1, CAS #19752-57-9) in DMSO (200 mL) was added tert-butyl piperazine-1-carboxylate (14.16 g, 76.01 mmol, CAS #57260-71-6), K₂CO₃ (19.1 g, 138 mmol), CuI (2.63 g, 13.8 mmol) and L-proline (3.18 g, 27.64 mmol). Then the reaction was stirred at 80° C. for 2 hrs under N₂ atmosphere. On completion, the reaction mixture was quenched with H₂O (150 mL) at 25° C. and then extracted with EA (100 mL×3). The combined organic layers were washed with brine (100 mL×2), dried over Na₂SO₄, filtered and the filtrate was concentrated under reduced pressure to give a residue. The residue was purified by column chromatography (SiO₂, Petroleum ether/Ethyl acetate=1/0 to 10/1) to give the title compound (15 g, 51% yield) as an orange solid. LC-MS (ESI⁺) m/z 421.0 (M+H)⁺.

Step 1—Tert-butyl 4-(3-bromo-5-((trimethylsilyl)ethynyl)phenyl)piperazine-1-carboxylate. A mixture of tert-butyl 4-(3,5-dibromophenyl)piperazine-1-carboxylate (3 g, 7.14 mmol), ethynyl(trimethyl)silane (351 mg, 3.57 mmol), Pd(PPh₃)₂Cl₂ (251 mg, 357 umol), CuI (136 mg, 714 umol) and TEA (1.08 g, 10.7 mmol, 1.49 mL) in THF (50 mL) was degassed and purged with N₂ three times. Then the mixture was stirred at 60° C. for 12 hrs under N₂ atmosphere. On completion, the reaction mixture was diluted with H₂O (50 mL) and extracted with EA 150 mL (50 mL×3). The combined organic layers were dried over Na₂SO₄, filtered and concentrated under reduced pressure to give a residue. The residue was purified by column chromatography (SiO₂, Petroleum ether/Ethyl acetate=1/0 to 10/1) to give the title compound (708 mg, 40% yield) as a yellow oil. LC-MS (ESI⁺) m/z 439.1 (M+H)⁺.

Step 2—Tert-butyl 4-(3-(4,4,5,5-tetramethyl-1,3,2-dioxaborolan-2-yl)-5-((trimethylsilyl)ethynyl)phenyl)piperazine-1-carboxylate. A mixture of tert-butyl 4-[3-bromo-5-(2-trimethylsilylethynyl)phenyl]piperazine-1-carboxylate (700 mg, 1.60 mmol), 4,4,5,5-tetramethyl-2-(4,4,5,5-tetramethyl-1,3,2-dioxaborolan-2-yl)-1,3,2-dioxaborolane (813 mg, 3.20 mmol), KOAc (471 mg, 4.80 mmol), Pd(dppf)Cl₂ (117 mg, 160 umol) in dioxane (8 mL) was degassed and purged with N₂ three times. Then the mixture was stirred at 80° C. for 2 hrs under N₂ atmosphere. On completion, the reaction mixture was diluted with H₂O (20 mL) and extracted with EA (20 mL×3). The combined organic layers were dried over Na₂SO₄, filtered and concentrated under reduced pressure to give a residue. The residue was purified by column chromatography (SiO₂, Petroleum ether/Ethyl acetate=1/0 to 5/1) to give the title compound (632 mg, 1.25 mmol, 78% yield) as a light yellow solid. LC-MS (ESI⁺) m/z 485.1 (M+H)⁺. ¹H NMR (400 MHz, DMSO-d₆) δ=7.18 (d, J=2.0 Hz, 1H), 7.14 (s, 1H), 7.09 (s, 1H), 3.43 (s, 4H), 3.14-3.08 (m, 4H), 1.42 (s, 9H), 1.28 (s, 12H), 0.22 (s, 9H).

### (R)-3-(3-ethynyl-5-(piperazin-1-yl)phenyl)-10-methyl-9,10,11,12-tetrahydro-8H-[1,4]diazepino[5′,6′:4,5]thieno[3,2-f]quinolin-8-one (Intermediate CA)

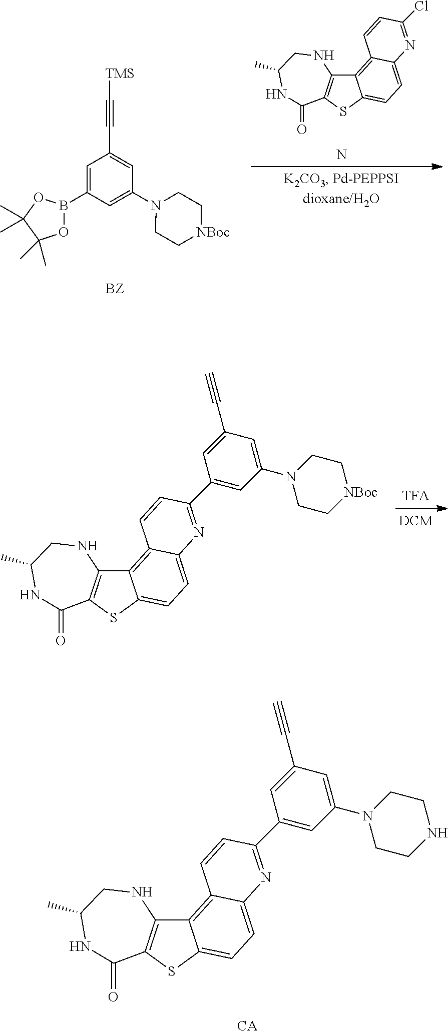

Step 1—(R)-tert-butyl 4-(3-ethynyl-5-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. A mixture of tert-butyl 4-[3-(4,4,5,5-tetramethyl-1,3,2-dioxaborolan-2-yl)-5-(2-trimethylsilylethynyl)phenyl]piperazine-1-carboxylate (290 mg, 599 umol, Intermediate BZ), (15R)-5-chloro-15-methyl-11-thia-6,14,17-triazatetracyclo[8.8.0.02,7.012,18]octadeca-1,3,5,7,9,12(18)-hexaen-13-one (190 mg, 599 umol Intermediate N), K₂CO₃ (248 mg, 1.80 mmol), and Pd-PEPPSI-IHeptCl (58.2 mg, 59.9 umol CAS #1814936-54-3) in dioxane (6 mL) and H₂O (3 mL) was degassed and purged with N₂ three times. Then the mixture was stirred at 80° C. for 2 hrs under N₂ atmosphere. On completion, the reaction mixture was filtered to give a residue. The residue was purified by prep-HPLC (FA condition) to give the title compound (36 mg, 10% yield, FA) as a yellow solid. LC-MS (ESI⁺) m/z 568.5 (M+H)⁺.

Step 2—(R)-3-(3-ethynyl-5-(piperazin-1-yl)phenyl)-10-methyl-9,10,11,12-tetrahydro-8H-[1,4]diazepino[5′,6′:4,5]thieno[3,2-f]quinolin-8-one. To a solution of tert-butyl 4-[3-ethynyl-5-[(15R)-15-methyl-13-oxo-11-thia-6,14,17-triazatetracyclo[8.8.0.02,7.012,18]octadeca-1,3,5,7,9,12(18)-hexaen-5-yl]phenyl]piperazine-1-carboxylate (36 mg, 58.66 umol, FA) in DCM (4 mL) was added TFA (1.54 g, 13.5 mmol, 1 mL). The mixture was stirred at 25° C. for 2 hrs. On completion, the reaction mixture was concentrated under reduced pressure to give the title compound (36 mg, TFA) as a red solid. LC-MS (ESI⁺) m/z 468.4 (M+H)⁺. ¹H NMR (400 MHz, DMSO-d₆) δ=13.68-12.91 (m, 1H), 9.69 (s, 1H), 8.34 (s, 1H), 8.16 (s, 1H), 7.93 (s, 1H), 7.81 (d, J=6.0 Hz, 1H), 7.61 (s, 1H), 7.55 (d, J=8.4 Hz, 1H), 7.25 (s, 1H), 7.05 (dd, J=1.6, 8.4 Hz, 1H), 6.77 (s, 2H), 6.11 (d, J=6.0 Hz, 1H), 3.52 (s, 4H), 3.21-3.17 (m, 4H), 1.44 (s, 9H).

### 1-(4-(3-(4-methoxybenzyl)-2,4-dioxotetrahydropyrimidin-1(2H)-yl)isoquinolin-8-yl)azetidine-3-carbaldehyde (Intermediate CB)

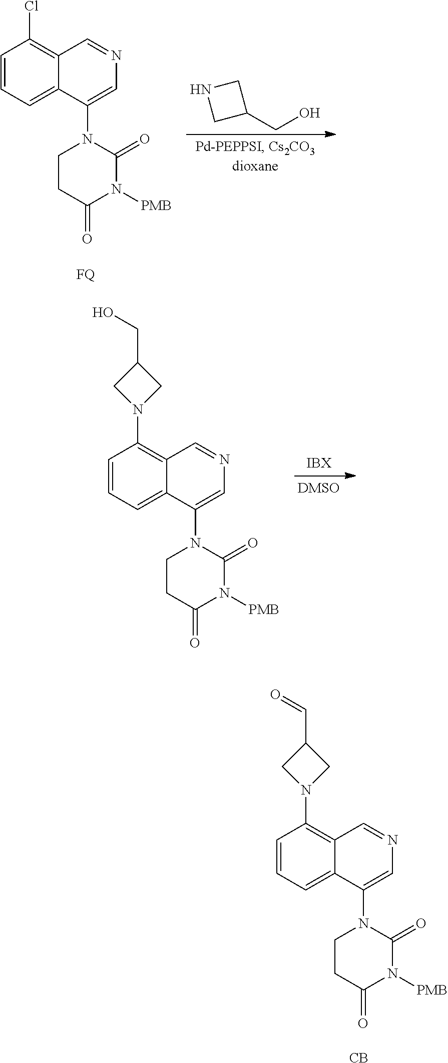

Step 1—1-(8-(3-(hydroxymethyl)azetidin-1-yl)isoquinolin-4-yl)-3-(4-methoxybenzyl)dihydropyrimidine-2,4(1H,3H)-dione. To a solution of 1-(8-chloroisoquinolin-4-yl)-3-(4-methoxybenzyl)dihydropyrimidine-2,4(1H,3H)-dione (500 mg, 1.26 mmol, Intermediate FQ) and azetidin-3-ylmethanol (165 mg, 1.89 mmol, CAS #928038-44-2) in dioxane (5 mL) was added Pd-PEPPSI-IHeptCl (123 mg, 126 umol) and Cs₂CO₃ (1.23 g, 3.79 mmol) at 25° C., then the mixture was stirred at 100° C. for 17 hrs. On completion, the reaction mixture was filtered and concentrated in vacuo to give the crude residue. The residue was purified by column chromatography (SiO₂, DCM:MeOH=70:1 to 30:1) to give the title compound (200 mg, 32% yield) as a white solid. LC-MS (ESI+) m/z 447.1. (M+H)⁺.

Step 2—1-(4-(3-(4-methoxybenzyl)-2,4-dioxotetrahydropyrimidin-1(2H)-yl)isoquinolin-8-yl)azetidine-3-carbaldehyde. To a solution of 1-(8-(3-(hydroxymethyl)azetidin-1-yl)isoquinolin-4-yl)-3-(4-methoxybenzyl)dihydropyrimidine-2,4(1H,3H)-dione (200 mg, 447 umol) in DMSO (2 mL) was added IBX (250 mg, 895 umol) at 25° C., the mixture was stirred at 25° C. for 2 hrs. On completion, the reaction mixture was filtered and concentrated in vacuo to get the crude residue. The crude product was purified by reversed-phase HPLC (0.1% FA condition) to give the title compound (90 mg, 37% yield) as a white solid. LC-MS (ESI⁺) m/z 463.0. (M+H)⁺.

### 3-(1-(2,6-dioxopiperidin-3-yl)-3-methyl-2-oxo-2,3-dihydro-1H-benzo[d]imidazol-4-yl)propanal (Intermediate CC)

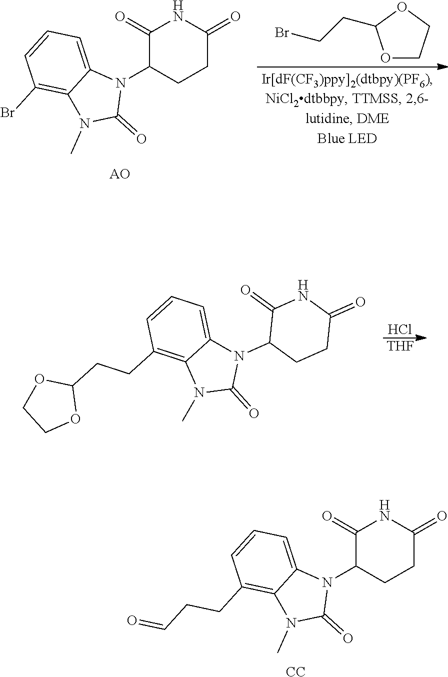

Step 1—3-(4-(2-(1,3-Dioxolan-2-yl)ethyl)-3-methyl-2-oxo-2,3-dihydro-1H-benzo[d]imidazol-1-yl)piperidine-2,6-dione. To an 15 mL vial equipped with a stir bar was added 3-(4-bromo-3-methyl-2-oxo-benzimidazol-1-yl)piperidine-2,6-dione (1 g, 2.96 mmol, Intermediate AO), 2-(2-bromoethyl)-1,3-dioxolane (696 mg, 3.84 mmol, 461 uL, CAS #18742-02-4), Ir[dF(CF₃)ppy]₂(dtbpy)(PF6) (66.4 mg, 59.1 umol) NiCl2.dtbbpy (23.5 mg, 59.1 umol), TTMSS (735 mg, 2.96 mmol, 912 uL), and 2,6-lutidine (634 mg, 5.91 mmol, 689 uL) in DME (5 mL). The vial was sealed and placed under nitrogen. The reaction was stirred and irradiated with a 10 W blue LED lamp (3 cm away), with cooling water to keep the reaction temperature at 25° C. for 14 hs. On completion, the reaction mixture was diluted with H₂O (20 mL) and extracted with EA (20 mL×3). The combined organic layers were dried over Na₂SO₄, filtered and concentrated under reduced pressure to give a residue. The residue was purified by column choursomatography (SiO₂, Petroleum ether/Ethyl acetate=2/1 to 0/1) to give the title compound (310 mg, 28% yield) as a yellow solid. LC-MS (ESI⁺) m/z 360.2 (M+1)⁺.

Step 2—3-(1-(2,6-dioxopiperidin-3-yl)-3-methyl-2-oxo-2,3-dihydro-1H-benzo[d]imidazol-4-yl)propanal. To a solution of 3-[4-[2-(1,3-dioxolan-2-yl)ethyl]-3-methyl-2-oxo-benzimidazol-1-yl]piperidine-2,6-dione (150 mg, 417 umol) in THF (2 mL) was added HCl (1 M, 1.25 mL). The mixture was stirred at 50° C. for 2 hrs. On completion, the reaction mixture was diluted with H₂O (5 mL) and extracted with EA (5 mL×4). The combined organic layers were dried over Na₂SO₄, filtered and concentrated under reduced pressure to give the title compound (100 mg, 76% yield) as a white solid LC-MS (ESI⁺) m/z 316.0 (M+1)⁺.

### (S)-3-(((tert-butyldimethylsilyl)oxy)methyl)pyrrolidine (Intermediate CD)

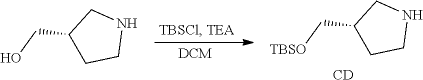

To a solution of [(3S)-pyrrolidin-3-yl]methanol (4 g, 39.6 mmol) and TEA (12.0 g, 119 mmol, 16.5 mL) in DCM (40 mL) was added TBSCl (7.15 g, 47.5 mmol, 5.82 mL). The mixture was stirred at 25° C. for 12 hrs. On completion, the reaction mixture was quenched with H₂O (100 mL) at 25° C., and then extracted with DCM (100 mL×3). The combined organic layers were washed with NaCl (100 mL×3), dried over Na₂SO₄, filtered and concentrated under reduced pressure to give a residue. The residue was purified by or by prep-TLC (SiO₂, DCM:MeOH=4:1) to give a compound (3 g, 25% yield) as a light brown liquid. LC-MS (ESI⁺) m/z 216.3. (M+H)⁺.

### ((3S)-1-(1-(1-(4-methoxybenzyl)-2,6-dioxopiperidin-3-yl)-3-methyl-2-oxo-2,3-dihydro-1H-benzo[d]imidazol-5-yl)pyrrolidin-3-yl)methyl 4-methylbenzenesulfonate (Intermediate CE)

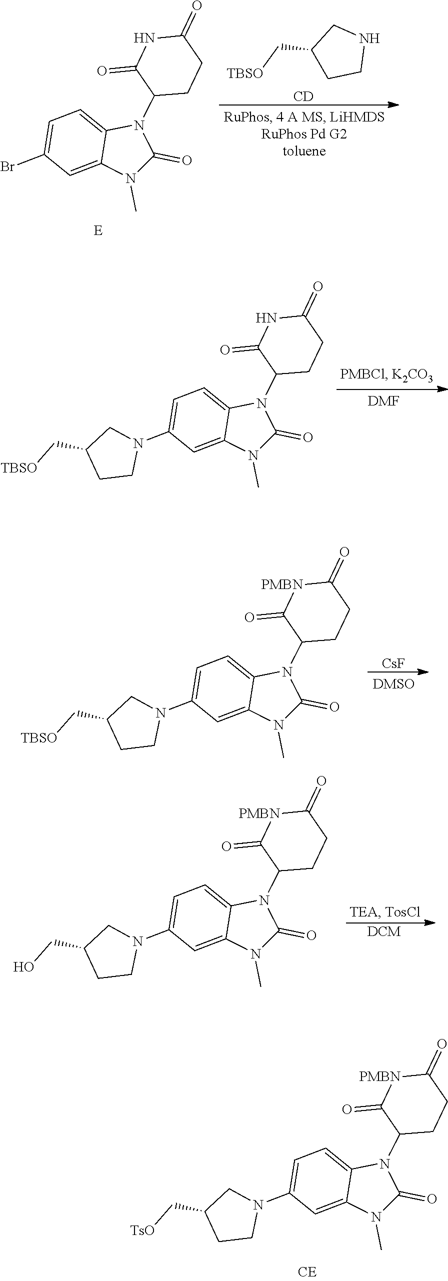

Step 1—3-(5-((S)-3-(((tert-butyldimethylsilyl)oxy)methyl)pyrrolidin-1-yl)-3-methyl-2-oxo-2,3-dihydro-1H-benzo[d]imidazol-1-yl)piperidine-2,6-dione. To a solution of 3-(5-bromo-3-methyl-2-oxo-benzimidazol-1-yl)piperidine-2,6-dione (4.28 g, 12.7 mmol, Intermediate E) in toluene (40 mL) was added tert-butyl-dimethyl-[[(3S)-pyrrolidin-3-yl]methoxy]silane (3 g, 13.9 mmol, Intermediate CD) and 4 Å molecular sieves (500 mg) and then purged with N₂ three times. Then [2-(2-aminophenyl)phenyl]-chloro-palladium;dicyclohexyl-[2-(2,6-diisopropoxyphenyl)phenyl]phosphane (983 mg, 1.27 mmol), RuPhos (591 mg, 1.27 mmol) and LiHMDS (1 M, 63.3 mL) was added and the mixture was purged with N₂ three times. The mixture was stirred at 100° C. for 1 hr under N₂ atmosphere. On completion, the reaction mixture was concentrated under reduced pressure to give a residue. The residue was purified by column chromatography (SiO₂, Petroleum ether/Ethyl acetate=1/1 to 0/1) to give a compound (2.89 g, 45% yield) as a brown solid. LC-MS (ESI+) m/z 473.3 (M+H)⁺.

Step 2—3-(5-((S)-3-(((tert-butyldimethylsilyl)oxy)methyl)pyrrolidin-1-yl)-3-methyl-2-oxo-2,3-dihydro-1H-benzo[d]imidazol-1-yl)-1-(4-methoxybenzyl)piperidine-2,6-dione. To a solution of 3-[5-[(3S)-3-[[tert-butyl(dimethyl)silyl]oxymethyl]pyrrolidin-1-yl]-3-methyl-2-oxo-benzimidazol-1-yl]piperidine-2,6-dione (2.1 g, 4.44 mmol) in DMF (2 mL) was added PMB-Cl (696 mg, 4.44 mmol, 605 uL) and K₂CO₃ (1.23 g, 8.89 mmol). The reaction was stirred at 20° C. for 12 hrs. On completion, the reaction mixture was filtered and quenched with H₂O (20 mL) at 25° C., and then extracted with EtOAc (30 mL×3). The combined organic layers were washed with brine (25 mL×2), dried over Na₂SO₄, filtered and concentrated under reduced pressure to give the compound (3.2 g) as a dark brown solid. LC-MS (ESI⁺) m/z 593.2. (M+H)⁺.

Step 3—3-(5-((S)-3-(hydroxymethyl)pyrrolidin-1-yl)-3-methyl-2-oxo-2,3-dihydro-1H-benzo[d]imidazol-1-yl)-1-(4-methoxybenzyl)piperidine-2,6-dione. To a solution of 3-[5-[(3S)-3-[[tert-butyl(dimethyl)silyl]oxymethyl]pyrrolidin-1-yl]-3-methyl-2-oxo-benzimidazol-1-yl]-1-[(4-methoxyphenyl)methyl]piperidine-2,6-dione (3 g, 5.06 mmol) in DMSO (4 mL) was added CsF (1.54 g, 10.2 mmol, 373 uL). The reaction was stirred at 50° C. for 2 hrs. On completion, the reaction mixture was concentrated under reduced pressure to give a residue. The crude product was purified by reversed-phase HPLC (0.1% NH₄HCO₃ condition) to give a compound (1 g, 40% yield) as a white solid. LC-MS (ESI⁺) m/z 479.0. (M+H)⁺.

Step 4—((3S)-1-(1-(1-(4-methoxybenzyl)-2,6-dioxopiperidin-3-yl)-3-methyl-2-oxo-2,3-dihydro-1H-benzo[d]imidazol-5-yl)pyrrolidin-3-yl)methyl 4-methylbenzenesulfonate. To a solution of 3-[5-[(3S)-3-(hydroxymethyl)pyrrolidin-1-yl]-3-methyl-2-oxo-benzimidazol-1-yl]-1-[(4-methoxyphenyl)methyl]piperidine-2,6-dione (500 mg, 1.04 mmol) in DCM (2 mL) was added TEA (317 mg, 3.13 mmol, 436 uL) and TosCl (598 mg, 3.13 mmol). The mixture was stirred at 25° C. for 12 hrs. On completion, the reaction mixture concentrated under reduced pressure to give a residue. The residue was purified by column chromatography (SiO₂, Petroleum ether/Ethyl acetate=2/1 to 0/1) to give a compound (440 mg, 63% yield) as a gray solid. LC-MS (ESI⁺) m/z 632.8. (M+H)⁺.

### (R)-6-methyl-2-(6′-(piperazin-1-yl)-[2,3′-bipyridin]-4-yl)-6,7-dihydrofuro[3,2-c]pyridin-4(5H)-one (Intermediate CF)

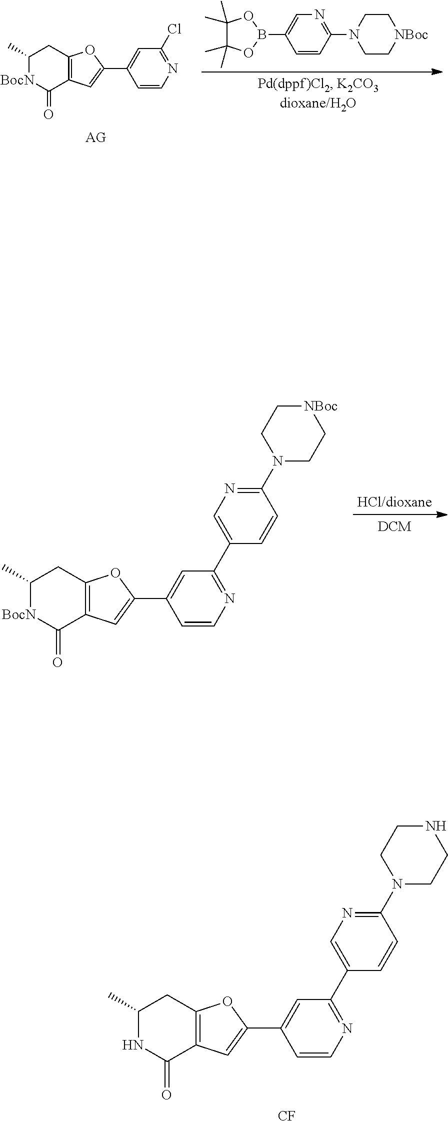

Step 1—(R)-tert-butyl 2-(6′-(4-(tert-butoxycarbonyl)piperazin-1-yl)-[2,3′-bipyridin]-4-yl)-6-methyl-4-oxo-6,7-dihydrofuro[3,2-c]pyridine-5(4H)-carboxylate. A mixture of tert-butyl (6R)-2-(2-chloro-4-pyridyl)-6-methyl-4-oxo-6,7-dihydrofuro[3,2-c]pyridine-5-carboxylate (115 mg, 317 umol Intermediate AG), tert-butyl 4-[5-(4,4,5,5-tetramethyl-1,3,2-dioxaborolan-2-yl)-2-pyridyl]piperazine-1-carboxylate (185 mg, 475 umol, CAS #496786-98-2), K₂CO₃ (131 mg, 951 umol), and Pd(dppf)Cl₂ (23.2 mg, 31.7 umol) in dioxane (2 mL) and H₂O (1 mL) was degassed and purged with N₂ three times. Then the mixture was stirred at 80° C. for 2 hrs under N₂ atmosphere. On completion, the reaction mixture was added 0.6 g silica powder, and concentrated under reduced pressure to give a residue. The residue was purified by column chromatography (SiO₂, Petroleum ether/Ethyl acetate=1/0 to 1/4) to give the title compound (159 mg, 82% yield) as a light yellow solid. LC-MS (ESI⁺) m/z 590.5 (M+H)⁺.

Step 2—(R)-6-methyl-2-(6′-(piperazin-1-yl)-[2,3′-bipyridin]-4-yl)-6,7-dihydrofuro[3,2-c]pyridin-4(5H)-one. To a solution of tert-butyl (6R)-2-[2-[6-(4-tert-butoxycarbonylpiperazin-1-yl)-3-pyridyl]-4-pyridyl]-6-methyl-4-oxo-6,7-dihydrofuro[3,2-c]pyridine-5-carboxylate (50 mg, 84.8 umol) in DCM (8 mL) was added HCl/dioxane (4 M, 2 mL). The mixture was stirred at 25° C. for 1 hr. On completion, the reaction mixture was concentrated under reduced pressure to give the title compound (50 mg, HCl) as an orange solid. LC-MS (ESI⁺) m/z 390.2 (M+H)⁺.

### 1-(4-(4-(4-oxo-4,5,6,7-tetrahydro-1H-pyrrolo[3,2-c]pyridin-2-yl)pyridin-2-yl)phenyl)piperidine-4-carbaldehyde (Intermediate CG)

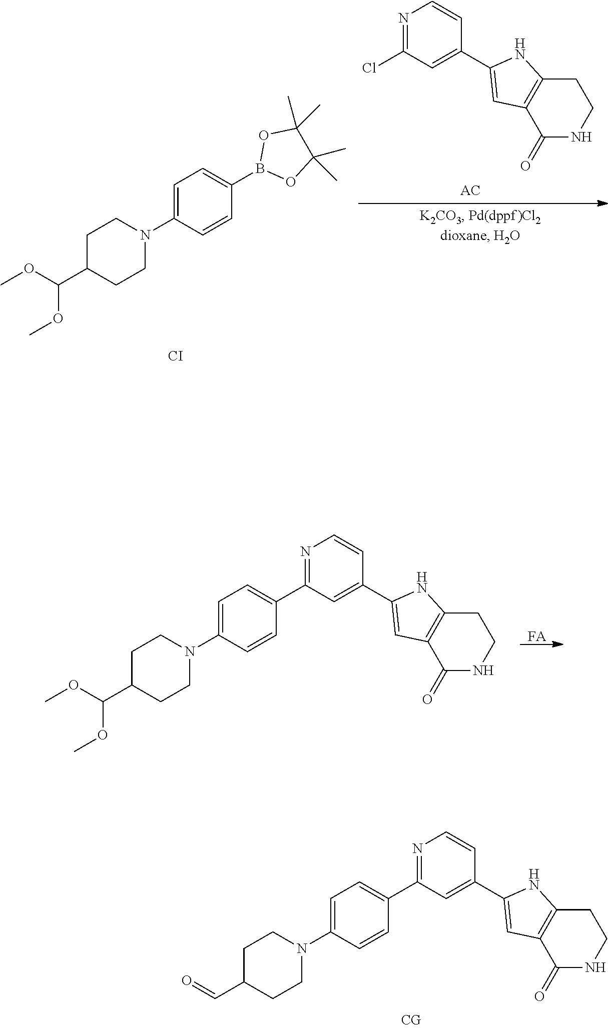

Step 1—2-(2-(4-(4-(Dimethoxymethyl)piperidin-1-yl)phenyl)pyridin-4-yl)-6,7-dihydro-1H-pyrrolo[3,2-c]pyridin-4(5H)-one. A mixture of 4-(dimethoxymethyl)-1-(4-(4,4,5,5-tetramethyl-1,3,2-dioxaborolan-2-yl)phenyl)piperidine (400 mg, 1.11 mmol, CAS #2242744-56-3, Intermediate CI), 2-(2-chloropyridin-4-yl)-6,7-dihydro-1H-pyrrolo[3,2-c]pyridin-4(5H)-one (182 mg, 738 umol, Intermediate AC), Pd(dppf)Cl₂ (54.0 mg, 73.8 umol), Cs₂CO₃ (721 mg, 2.21 mmol) in dioxane (5 mL) and H₂O (1 mL) was degassed and purged with N₂ three times. Then the mixture was stirred at 100° C. for 2 hrs under N₂ atmosphere. The reaction mixture was diluted with H₂O (10 mL) and extracted with EA (10 mL×3). The combined organic layers were washed with brine (10 mL×3), dried over Na₂SO₄, filtered and concentrated under reduced pressure to give a residue. The crude product was triturated with PE at 25° C. for 10 min to give the title compound (162 mg, 40% yield) as a white solid. LC-MS (ESI⁺) m/z 447.3 (M+H)⁺.

Step 2—1-(4-(4-(4-Oxo-4,5,6,7-tetrahydro-1H-pyrrolo[3,2-c]pyridin-2-yl)pyridin-2-yl)phenyl)piperidine-4-carbaldehyde. A solution of 2-(2-(4-(4-(dimethoxymethyl)piperidin-1-yl)phenyl)pyridin-4-yl)-6,7-dihydro-1H-pyrrolo[3,2-c]pyridin-4(5H)-one (80 mg, 179 umol) in FA (1 mL) was stirred at 40° C. for 1 hrs. The reaction mixture was concentrated under reduced pressure to give the title compound (70 mg) as a brownish black oil. LC-MS (ESI⁺) m/z 401.2 (M+H)⁺.

### 1-(8-Piperazin-1-yl-4-isoquinolyl)hexahydropyrimidine-2,4-dione (Intermediate CH)

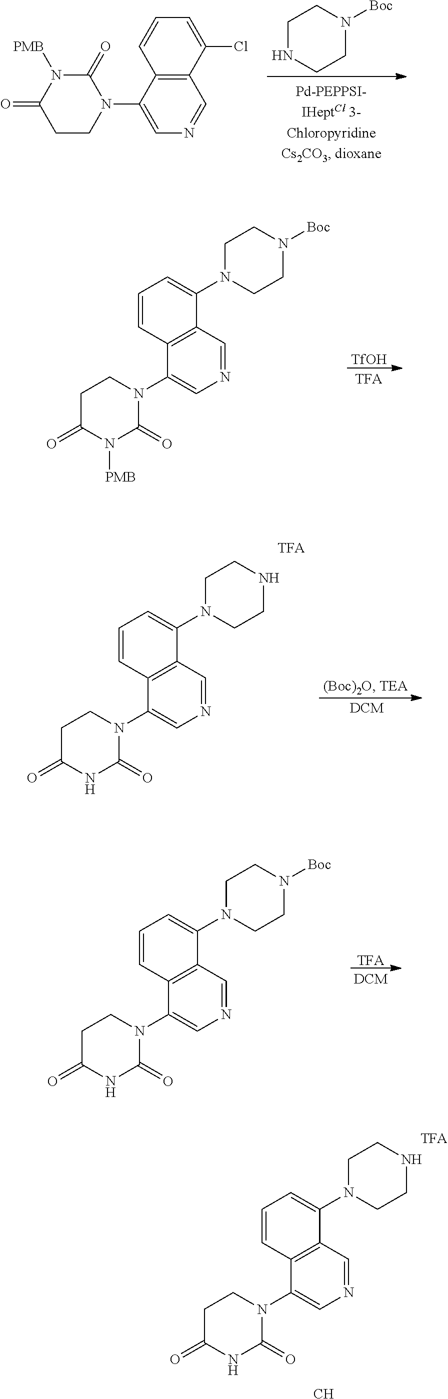

Step 1—Tert-butyl 4-[4-[3-[(4-methoxyphenyl)methyl]-2,4-dioxo-hexahydropyrimidin-1-yl]-8- isoquinolyl]piperazine-1-carboxylate. To a solution of 1-(8-chloro-4-isoquinolyl)-3-[(4-methoxyphenyl)methyl]hexahydropyrimidine-2,4-dione (900 mg, 2.27 mmol, synthesized via Steps 1-2 of Intermediate FQ) and tert-butyl piperazine-1-carboxylate (508 mg, 2.73 mmol, CAS #143238-38-4) in dioxane (15 mL) was added Pd-PEPPSI-IHeptCl 3-Chloropyridine (221 mg, 227 umol) and Cs₂CO₃ (1.48 g, 4.55 mmol), then the mixture was stirred at 80° C. for 8 hours. On completion, the mixture was filtered and concentrated in vacuo to give the title compound (1.20 g, 96% yield) as a brown solid. ¹H NMR (400 MHz, CDCl₃-d) δ 9.52 (s, 1H), 8.40 (s, 1H), 7.56 (t, J=8.0 Hz, 1H), 7.37 (d, J=8.8 Hz, 2H), 7.23-7.19 (m, 1H), 7.09 (d, J=7.6 Hz, 1H), 6.78 (d, J=8.4 Hz, 2H), 4.94 (s, 2H), 3.85-3.75 (m, 2H), 3.74 (s, 3H), 3.70-3.64 (m, J=6.4, 12.4 Hz, 4H), 3.34-3.31 (m, 1H), 3.13-3.07 (m, 2H), 2.97-2.93 (m, 2H), 2.76-2.73 (m, 1H), 1.45 (s, 9H). LC-MS (ESI⁺) m/z 546.6 (M+H)⁺.

Step 2—1-(8-Piperazin-1-yl-4-isoquinolyl)hexahydropyrimidine-2,4-dione. To a solution of tert-butyl 4-[4-[3-[(4-methoxyphenyl)methyl]-2,4-dioxo-hexahydropyrimidin-1-yl]-8-isoquinolyl]piperazine-1-carboxylate (600 mg, 1.10 mmol) in TFA (6 mL) was added TfOH (1 mL), then the mixture was stirred at 70° C. for 2 hours. On completion, the mixture was concentrated in vacuo to give the title compound (480 mg, 99% yield, TFA) as a black brown solid. LC-MS (ESI⁺) m/z 326.1 (M+H)⁺.

Step 3—Tert-butyl 4-[4-(2,4-dioxohexahydropyrimidin-1-yl)-8-isoquinolyl]piperazine-1-carboxylate. To a solution of 1-(8-piperazin-1-yl-4-isoquinolyl)hexahydropyrimidine-2,4-dione (480 mg, 1.09 mmol, TFA) in DCM (5 mL) was added TEA (304 uL, 2.18 mmol) and Boc₂O (357 mg, 1.64 mmol), then the mixture was stirred at 25° C. for 10 hours. On completion, the mixture was diluted with DCM (300 mL) and extracted with water (200 mL×3). The combined organic layer was dried over anhydrous Na₂SO₄, filtered and concentrated in vacuo to give the residue. The residue was purified by reverse-phase (0.1% FA condition) to give the title compound (150 mg, 32% yield) as a yellow solid. ¹H NMR (400 MHz, CDCl₃-d) δ 9.61 (s, 1H), 8.52 (s, 1H), 8.26 (s, 1H), 7.69 (t, J=8.0 Hz, 1H), 7.47 (d, J=8.4 Hz, 1H), 7.18 (d, J=7.6 Hz, 1H), 4.03-3.91 (m, 1H), 3.90-3.77 (m, 2H), 3.73 (s, 3H), 3.23-3.02 (m, 4H), 3.01-2.88 (m, 2H), 1.51 (s, 9H). LC-MS (ESI⁺) m/z 426.1 (M+H)⁺.

Step 4—1-(8-Piperazin-1-yl-4-isoquinolyl)hexahydropyrimidine-2,4-dione. To a solution of tert-butyl 4-[4-(2,4-dioxohexahydropyrimidin-1-yl)-8-isoquinolyl]piperazine-1- carboxylate (80.0 mg, 188 umol) in DCM (2 mL) was added TFA (0.5 mL, 6.75 mmol), then the mixture was stirred at 25° C. for 1 hour. On completion, the mixture was concentrated in vacuo to give the title compound (80.0 mg, 96% yield, TFA) as a brown solid. LC-MS (ESI⁺) m/z 326.1 (M+H)⁺.

### 4-(Dimethoxymethyl)-1-(4-(4,4,5,5-tetramethyl-1,3,2-dioxaborolan-2-yl)phenyl)piperidine (Intermediate CI)

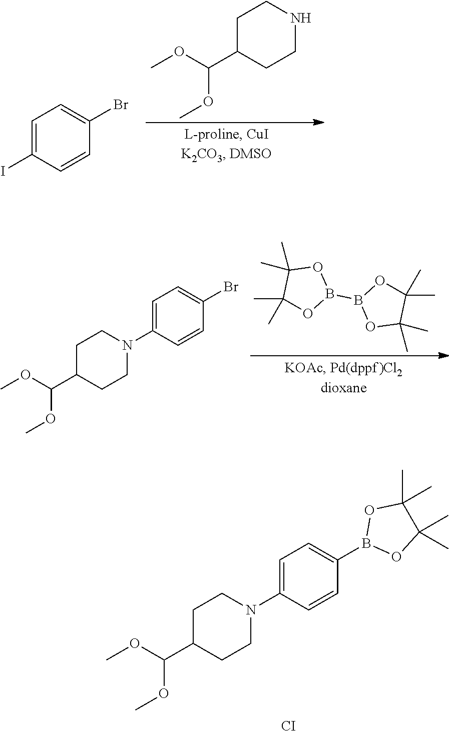

Step 1—1-(4-Bromophenyl)-4-(dimethoxymethyl)piperidine. A mixture of 1-bromo-4-iodobenzene (5 g, 17.7 mmol, CAS #589-87-7), 4-(dimethoxymethyl)piperidine (2.81 g, 17.7 mmol, CAS #188646-83-5), K₂CO₃ (4.89 g, 35.5 mmol), L-proline (813 mg, 7.07 mmol) and CuI (673 mg, 3.53 mmol) in DMSO (100 mL) was degassed and purged with N₂ three times. Then the mixture was stirred at 100° C. for 4 hrs under N₂ atmosphere. The reaction mixture was diluted with H₂O (50 mL) and extracted with EA (50 mL×3). The combined organic layers were washed with brine (50 mL×3), dried over Na₂SO₄, filtered and concentrated under reduced pressure to give a residue. The residue was purified by column chromatography (SiO₂, Petroleum ether/Ethyl acetate=10/1 to 5/1) to give the title compound (3.39 g, 45% yield) as a white solid. LC-MS (ESI⁺) m/z 316.0 (M+H)⁺.

Step 2—4-(Dimethoxymethyl)-1-(4-(4,4,5,5-tetramethyl-1,3,2-dioxaborolan-2-yl)phenyl)piperidine. A mixture of 1-(4-bromophenyl)-4-(dimethoxymethyl)piperidine (1.5 g, 4.77 mmol), 4,4,5,5-tetramethyl-2-(4,4,5,5-tetramethyl-1,3,2-dioxaborolan-2-yl)-1,3,2-dioxaborolane (2.42 g, 9.55 mmol), KOAc (1.41 g, 14.3 mmol), and Pd(dppf)Cl₂ (349 mg, 477 umol) in DMSO (25 mL) was degassed and purged with N₂ three times. Then the mixture was stirred at 80° C. for 2 hrs under N₂ atmosphere. The reaction mixture was diluted with H₂O (20 mL) and extracted with EA (20 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=10/1 to 5/1) to give the title compound (900 mg, 39% yield) as a white solid. LC-MS (ESI⁺) m/z 360.2 (M+H)⁺

### 1-(4-(4-(4′-oxo-1′,4′,5′,6′-tetrahydrospiro[cyclopropane-1,7′-pyrrolo[3,2-c]pyridin]-2′-yl)pyridin-2-yl)phenyl)piperidine-4-carbaldehyde (Intermediate CJ)

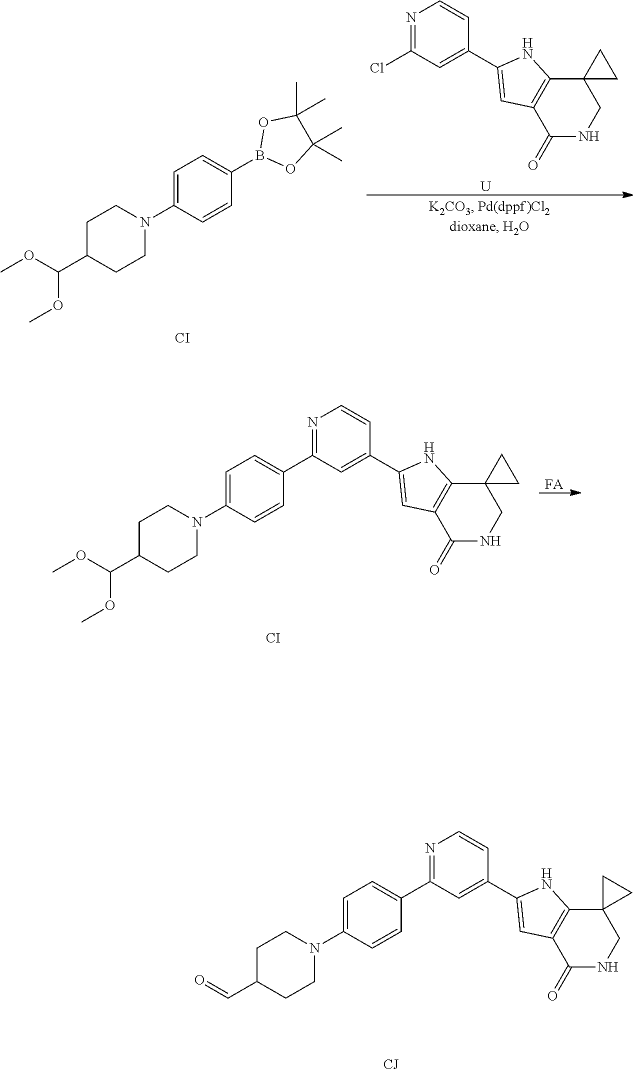

Step 1—2′-(2-(4-(4-(Dimethoxymethyl)piperidin-1-yl)phenyl)pyridin-4-yl)-5′,6′-dihydrospiro[cyclopropane-1,7′-pyrrolo[3,2-c]pyridin]-4′(1′H)-one. A mixture of 4-(dimethoxymethyl)-1-[4-(4,4,5,5-tetramethyl-1,3,2-dioxaborolan-2-yl)phenyl] piperidine (400 mg, 1.11 mmol, Intermediate CI), 2′-(2-chloropyridin-4-yl)-5′,6′-dihydrospiro[cyclopropane-1,7′-pyrrolo[3,2-c]pyridin]-4′(1′H)-one (233 mg, 851 umol, Intermediate U), Cs₂CO₃ (832 mg, 2.56 mmol) and Pd(dppf)Cl₂ (62.3 mg, 85.1 umol) in dioxane (6 mL) and H₂O (1.5 mL) was degassed and purged with N₂ three times. Then the mixture was stirred at 100° C. for 2 hrs under N₂ atmosphere. The reaction mixture was diluted with H₂O (10 mL) and extracted with EA (10 mL×3). The combined organic layers were washed with brine (10 mL×3), dried over Na₂SO₄, filtered and concentrated under reduced pressure to give a residue. The crude product was triturated with PE at 25° C. for 10 min to give the title compound (160 mg, 33% yield) as a white solid. LC-MS (ESI⁺) m/z 473.2 (M+H)⁺.

Step 2—1-(4-(4-(4′-Oxo-1′,4′,5′,6′-tetrahydrospiro[cyclopropane-1,7′-pyrrolo[3,2-c]pyridin]-2′-yl)pyridin-2-yl)phenyl)piperidine-4-carbaldehyde. A solution of 2′-(2-(4-(4-(dimethoxymethyl)piperidin-1-yl)phenyl)pyridin-4-yl)-5′,6′-dihydrospiro[cyclopropane-1,7′-pyrrolo[3,2-c]pyridin]-4′(1′H)-one (80 mg, 169 umol) in FA (1.5 mL) was stirred at 40° C. for 1 hr. On completion, the reaction mixture was concentrated under reduced pressure to give the title compound (70 mg) as a brownish black oil. LC-MS (ESI⁺) m/z 427.2 (M+H)⁺.

### 2′-(6′-(Piperazin-1-yl)-[2,3′-bipyridin]-4-yl)-5′,6′-dihydrospiro[cyclopropane-1,7′-pyrrolo[3,2-c]pyridin]-4′(1′H)-one (Intermediate CK)

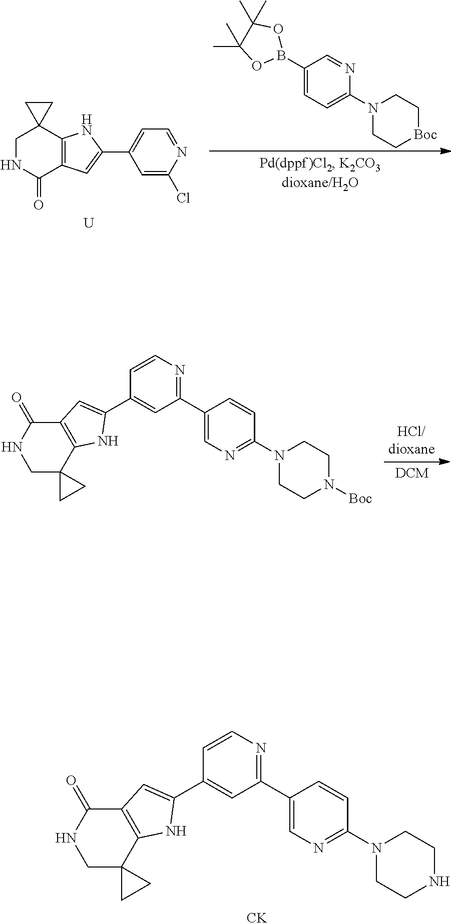

Step 1—Tert-butyl 4-(4-(4′-oxo-1′,4′,5′,6′-tetrahydrospiro[cyclopropane-1,7′-pyrrolo[3,2-c]pyridin]-2′-yl)-[2,3′-bipyridin]-6′-yl)piperazine-1-carboxylate. A mixture of 2-(2-chloro-4-pyridyl)spiro[5,6-dihydro-1H-pyrrolo[3,2-c]pyridine-7,1′-cyclopropane]-4-one (650 mg, 2.37 mmol, Intermediate U), tert-butyl 4-[5-(4,4,5,5-tetramethyl-1,3,2-dioxaborolan-2-yl)-2-pyridyl]piperazine-1-carboxylate (924 mg, 2.37 mmol, CAS #496786-98-2), Pd(dppf)Cl₂ (173.76 mg, 237.47 umol), and K₂CO₃ (985 mg, 7.12 mmol) in H₂O (1.5 mL) and dioxane (15 mL) was degassed and purged with N₂ three times. Then the mixture was stirred at 100° C. for 4 hrs under N₂ atmosphere. On completion, the reaction mixture was concentrated under reduced pressure to give a residue. The residue was purified by column chromatography (SiO₂, DCM:MeOH=1/0 to 10/1, R_{f}=0.28) to give the title compound (660 mg, 53% yield) as a brown solid. LC-MS (ESI⁺) m/z 501.2 (M+H)⁺.

Step 2—2′-(6′-(Piperazin-1-yl)-[2,3′-bipyridin]-4-yl)-5′,6′-dihydrospiro[cyclopropane-1,7′-pyrrolo[3,2-c]pyridin]-4′(1′H)-one. To a solution of tert-butyl 4-(4-(4′-oxo-1′,4′,5′,6′-tetrahydrospiro[cyclopropane-1,7′-pyrrolo[3,2-c]pyridin]-2′-yl)-[2,3′-bipyridin]-6′-yl)piperazine-1-carboxylate (300 mg, 599 umol) in DCM (5 mL) was added HCl/dioxane (4 M, 1 mL). The mixture was stirred at 25° C. for 2 hrs. On completion, the reaction mixture was concentrated under reduced pressure to give the title compound (360 mg, HCl) as a yellow solid. LC-MS (ESI⁺) m/z 401.3 (M+H)⁺.

### (R)-10-methyl-3-(3-(piperazin-1-yl)phenyl)-9,10,11,12-tetrahydro-8H-[1,4]diazepino[5′,6′:4,5]thieno[3,2-f]quinolin-8-one (Intermediate CL)

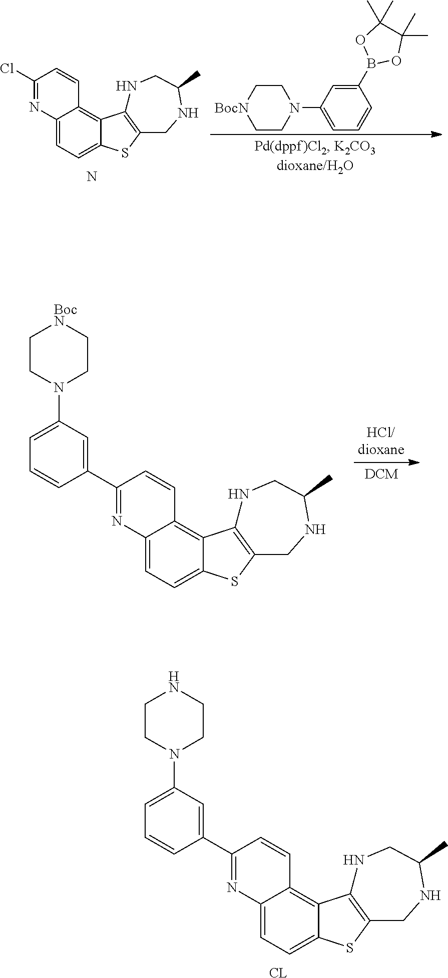

Step 1—(R)-tert-butyl 4-(3-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. A mixture of (15R)-5-chloro-15-methyl-1l-thia-6,14,17-triazatetracyclo[8.8.0.02,7.012,18]octadeca-1,3,5,7,9,12(18)-hexaen-13-one (500 mg, 1.57 mmol, Intermediate N), tert-butyl 4-[3-(4,4,5,5-tetramethyl-1,3,2-dioxaborolan-2-yl)phenyl]piperazine-1-carboxylate (916 mg, 2.36 mmol, CAS #540752-87-2), K₂CO₃ (652 mg, 4.72 mmol), and Pd(dppf)Cl₂ (115 mg, 157 umol) in dioxane (12 mL) and H₂O (4 mL) was degassed and purged with N₂ three times. Then the mixture was stirred at 80° C. for 2 hrs under N₂ atmosphere. On completion, the reaction was diluted with H₂O (30 mL) and extracted with EA (30 mL×4). The combined organic layers were dried over Na₂SO₄, filtered and concentrated under reduced pressure to give a residue. The residue was purified by column chromatography (SiO₂, Dichloromethane/Petroleum ether=0/1 to I/O)I to give the title compound (700 mg, 78% yield) as yellow solid. LC-MS (ESI⁺) m/z 544.3. (M+H)⁺.

Step 2—(R)-10-methyl-3-(3-(piperazin-1-yl)phenyl)-9,10,11,12-tetrahydro-8H-[1,4]diazepino[5′,6′:4,5]thieno[3,2-f]quinolin-8-one. To a solution of (R)-tert-butyl 4-(3-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (500 mg, 920 umol) in DCM (8 mL) was added HCl/dioxane (4 M, 2 mL), then the mixture was stirred at 25° C. for 2 hrs. On completion, the reaction mixture was concentrated in vacuo to get the crude residue. The crude product was purified by reversed-phase HPLC (column: Welch Xtimate C18 150×25 mm×5 um; mobile phase: [water (HCl)-ACN]; B %: 6%-36%, 9 min) to give the title compound (400 mg, 91% yield) as red solid. ¹H NMR (400 MHz, DMSO-d₆) δ=9.34 (d, J=8.4 Hz, 1H), 9.18 (s, 2H), 8.29 (d, J=8.8 Hz, 1H), 8.24-8.18 (m, 1H), 8.14 (d, J=9.2 Hz, 2H), 7.91 (s, 1H), 7.76 (d, J=7.6 Hz, 1H), 7.48 (t, J=7.6 Hz, 1H), 7.19 (dd, J=1.6, 8.4 Hz, 1H), 3.62-3.61 (m, 1H), 3.54-3.51 (m, 4H), 3.49 (s, 2H), 3.31-3.25 (m, 4H), 1.20 (d, J=6.8 Hz, 3H). LC-MS (ESI⁺) m/z 444.2. (M+H)⁺.

### Tert-butyl 4-(2,6-dimethyl-4-(4,4,5,5-tetramethyl-1,3,2-dioxaborolan-2-yl)phenyl) piperazine-1-carboxylate (Intermediate CM)

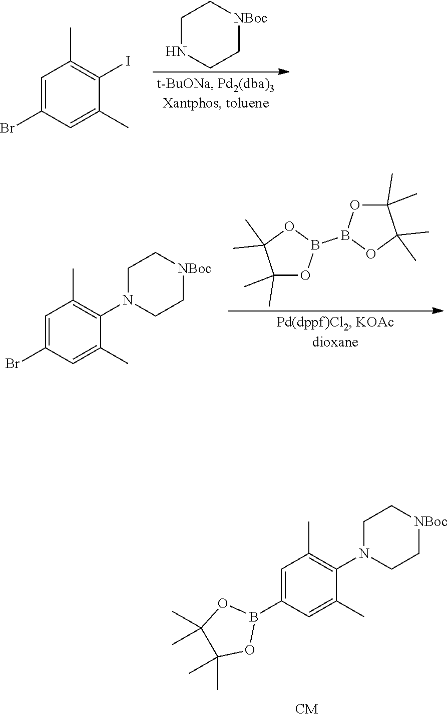

Step 1—Tert-butyl 4-(4-bromo-2,6-dimethylphenyl)piperazine-1-carboxylate. To a solution of 5-bromo-2-iodo-1,3-dimethylbenzene (5.0 g, 16 mmol, CAS #206559-43-5) and tert-butyl piperazine-1-carboxylate (3.6 g, 16 mmol, CAS #57260-71-6) in toluene (50 mL) was added t-BuONa (2.3 g, 24 mmol), Pd₂(dba)₃ (0.7 g, 0.8 mmol) and Xantphos (0.9 g, 1.6 mmol). Then the mixture was stirred at 60° C. for 12 hrs. The reaction mixture was quenched with H₂O (15 mL) at 25° C., and then diluted with EA (20 mL) and extracted with EA (20 mL×2). The combined organic layers were washed with sat. NaCl 30 mL (15 mL×2), dried over Na₂SO₄, filtered and concentrated under reduced pressure to give a residue. The residue was purified by column chromatography (SiO₂, Petroleum ether/Ethyl acetate=100/0 to 5/1) to give the title compound (0.6 g, 6.1% yield) as a white solid. LC-MS (ESI+) m/z 371.0 (M+1)⁺.

Step 2—Tert-butyl 4-(2,6-dimethyl-4-(4,4,5,5-tetramethyl-1,3,2-dioxaborolan-2-yl)phenyl) piperazine-1-carboxylate. To a solution of tert-butyl 4-(4-bromo-2,6-dimethylphenyl)piperazine-1-carboxylate (0.7 g, 2.0 mmol) in dioxane (10 mL) was added BPD (1.6 g, 6.1 mmol, CAS #73183-34-3), Pd(dppf)Cl₂ (0.1 g, 0.2 mmol) and KOAc (0.6 g, 6.1 mmol), then degassed and purged with N₂ three times. Then the mixture was stirred at 100° C. for 12 hrs under N₂ atmosphere. On completion, the reaction mixture was with H₂O (20 mL) at 25° C., and then diluted with ethyl acetate (20 mL) and extracted with ethyl acetate (20 mL×2). The combined organic layers were washed with sat. brine 20 mL (20 mL×1), dried over Na₂SO₄, filtered and concentrated under reduced pressure to give a residue. The residue was purified by column chromatography (SiO₂, PE/EA=30/1 to 1/1) to give the title compound (0.8 g, 1.9 mmol, 94% yield) as a white oil. LC-MS (ESI⁺) m/z 417.0 (M+1)⁺.

### 2-(2-(3,5-dimethyl-4-(piperazin-1-yl)phenyl)pyridin-4-yl)-6,7-dihydro-1H-pyrrolo[3,2-c]pyridin-4(5H)-one (Intermediate CN)

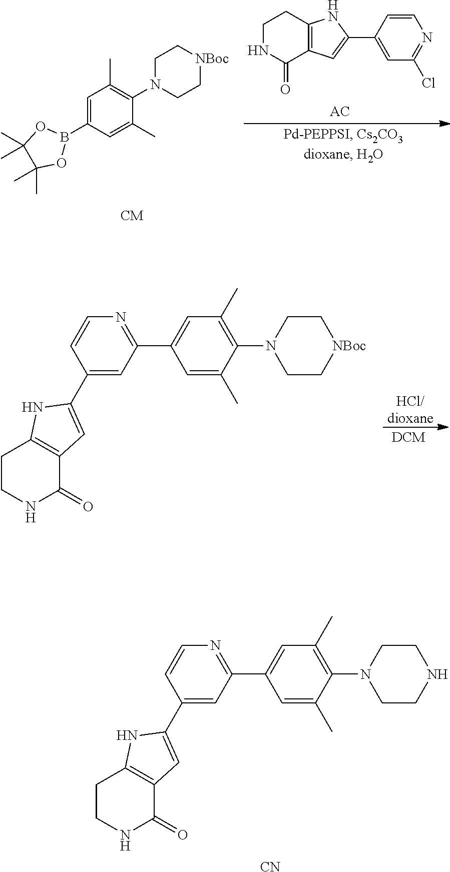 [SMILES: Cc1cc(B2OC(C)(C)C(C)(C)O2)cc(C)c1N1CCN(C(=O)OC(C)(C)C)CC1 ; O=C1NCCc2[nH]c(-c3ccnc(Cl)c3)cc21 ; Cc1cc(-c2cc(-c3cc4c([nH]3)CCNC4=O)ccn2)cc(C)c1N1CCN(C(=O)OC(C)(C)C)CC1 ; "HCl/dioxane" ; Cc1cc(-c2cc(-c3cc4c([nH]3)CCNC4=O)ccn2)cc(C)c1N1CCNCC1 ; "CN"]

Step 1—Tert-butyl 4-(2,6-dimethyl-4-(4-(4-oxo-4,5,6,7-tetrahydro-1H-pyrrolo[3,2-c]pyridin-2- yl)pyridin-2-yl)phenyl)piperazine-1-carboxylate. To a solution of tert-butyl 4-(2,6-dimethyl-4-(4,4,5,5-tetramethyl-1,3,2-dioxaborolan-2-yl)phenyl) piperazine-1-carboxylate (0.3 g, 0.7 mmol, Intermediate CM) and 2-(2-chloropyridin-4-yl)-6,7-dihydro-1H-pyrrolo[3,2-c]pyridin-4(5H)-one (0.1 g, 0.4 mmol, Intermediate AC) in dioxane (3.0 mL) and H₂O (0.5 mL) was added Pd-PEPPSI (41 mg, 42 umol) and Cs₂CO₃ (0.4 g, 1.3 mmol), then the mixture was stirred at 80° C. for 2 hrs. The reaction mixture was quenched with H₂O (20 mL0 at 25° C., and then diluted with ethyl acetate (20 mL) and extracted with ethyl acetate (20 mL×2). The combined organic layers were washed with sat. brine (20 mL×1), dried over Na₂SO₄, filtered and concentrated under reduced pressure to give a residue. The residue was purified by column chromatography (SiO₂, Dichloromethane:Methanol=30:1 to 5:1) to give the title compound (70 mg, 8% yield) as a yellow solid. LC-MS (ESI⁺) m/z 502.1 (M+1)⁺.

Step 2—2-(2-(3,5-Dimethyl-4-(piperazin-1-yl)phenyl)pyridin-4-yl)-6,7-dihydro-1H-pyrrolo[3,2-c]pyridin-4(5H)-one. To a solution of tert-butyl 4-(2,6-dimethyl-4-(4-(4-oxo-4,5,6,7-tetrahydro-1H-pyrrolo[3,2-c]pyridine-2-yl)pyridin-2-yl)phenyl)piperazine-1-carboxylate (0.2 g, 0.4 mmol) in DCM (2.0 mL) was added HCl/dioxane (4.0 M, 2.0 mL), the mixture was then stirred at 25° C. for 2.0 hrs. On completion, the reaction mixture was directly concentrated under reduced pressure to give the title compound (160 mg) as a yellow solid. LC-MS (ESI+) m/z 402.2 (M+1)⁺.

### 1-[4-[3-[(4-methoxyphenyl)methyl]-2,4-dioxo-hexahydropyrimidin-1-yl]-8-isoquinolyl]piperidine-4-carbaldehyde (Intermediate CO)

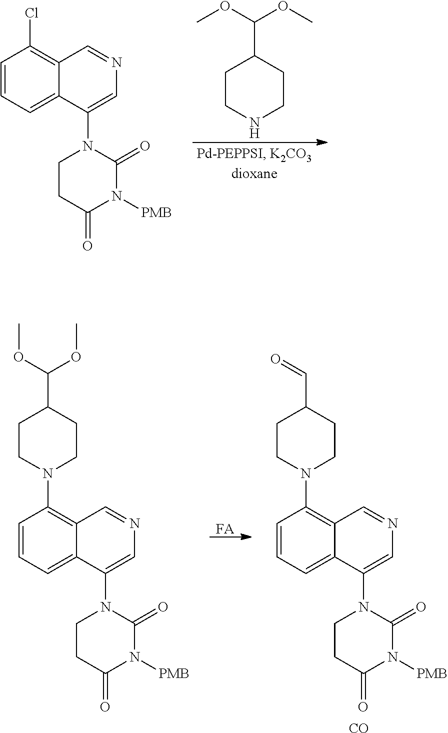

Step 1—1-(8-(4-(Dimethoxymethyl)piperidin-1-yl)isoquinolin-4-yl)-3-(4-methoxybenzyl)dihydropyrimidine-2,4(1H,3H)-dione. A mixture of 1-(8-chloro-4-isoquinolyl)-3-[(4-methoxyphenyl)methyl]hexahydropyrimidine-2,4-dione (500 mg, 1.26 mmol, Intermediate FQ), 4-(dimethoxymethyl)piperidine (301 mg, 1.89 mmol), 1,3-bis[2,6-bis(1-propylbutyl)phenyl]-4,5-dichloro-2H-imidazol-1-ium-2-ide;3-chloropyridine; dichloropalladium (122 mg, 126 umol), and Cs₂CO₃ (1.23 g, 3.79 mmol) in dioxane (10 mL) was degassed and purged with N₂ three times. Then the mixture was stirred at 100° C. for 4 hrs under N₂ atmosphere. On completion, the reaction mixture was concentrated under reduced pressure to remove solvent and was purified by column chromatography (SiO₂, Petroleum ether/Ethyl acetate=1/1 to 0/1, Rf=0.1) to give the title compound (600 mg, 91% yield) as a white solid. LC-MS (ESI⁺) m/z 519.2 (M+H)⁺.

Step 2—1-(4-(3-(4-Methoxybenzyl)-2,4-dioxotetrahydropyrimidin-1(2H)-yl)isoquinolin-8-yl)piperidine-4-carbaldehyde. A solution of 1-[8-[4-(dimethoxymethyl)-1-piperidyl]-4-isoquinolyl]-3-[(4-methoxyphenyl)methyl]hexahydropyrimidine-2,4-dione (300 mg, 578 umol) in formic acid (26.6 mg, 578 umol, 21.8 uL) was stirred at 25° C. for 0.3 hr. On completion, the reaction mixture was concentrated under reduced pressure to give the title compound (290 mg, FA) as yellow oil. LC-MS (ESI⁺) m/z 473.2 (M+H)⁺.

### 2′-(6′-(Piperazin-1-yl)-[2,3′-bipyridin]-4-yl)-5′,6′-dihydrospiro[cyclobutane-1,7′-pyrrolo[3,2-c]pyridin]-4′(1′H)-one (Intermediate CP)

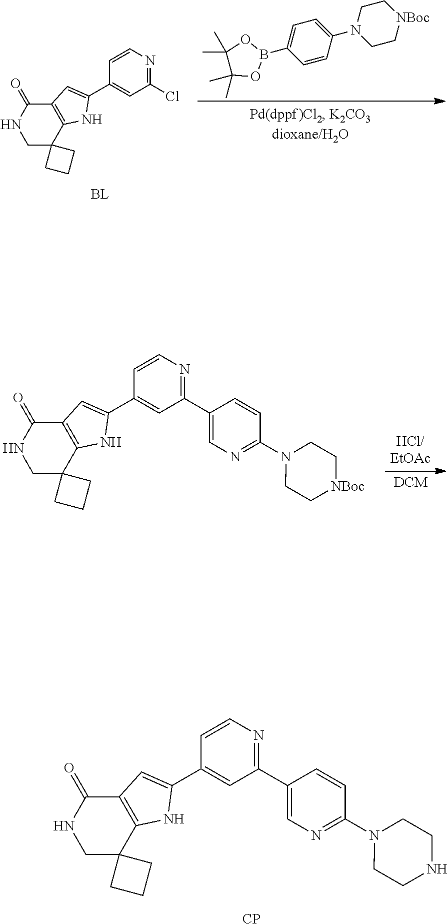 [SMILES: O=C1NCC2(CCC2)c2[nH]c(-c3ccnc(Cl)c3)cc21 ; CC(C)(C)OC(=O)N1CCN(c2ccc(B3OC(C)(C)C(C)(C)O3)cc2)CC1 ; CC(C)(C)OC(=O)N1CCN(c2ccc(-c3cc(-c4cc5c([nH]4)C4(CCC4)CNC5=O)ccn3)cn2)CC1 ; O=C1NCC2(CCC2)c2[nH]c(-c3ccnc(-c4ccc(N5CCNCC5)nc4)c3)cc21 ; "CP"]

Step 1—Tert-butyl 4-(4-(4′-oxo-1′,4′,5′,6′-tetrahydrospiro[cyclobutane-1,7′-pyrrolo[3,2-c]pyridin]-2′-yl)-[2,3′-bipyridin]-6′-yl)piperazine-1-carboxylate. To a solution of 2′-(2-chloropyridin-4-yl)-5′,6′-dihydrospiro[cyclobutane-1,7′-pyrrolo[3,2-c]pyridin]-4′(1′H)-one (150 mg, 521 umol, Intermediate BL) in dioxane (3 mL) and H₂O (0.75 mL) was added tert-butyl 4-(5-(4,4,5,5-tetramethyl-1,3,2-dioxaborolan-2-yl)pyridin-2-yl)piperazine-1-carboxylate (244 mg, 626 umol, CAS #496786-98-2), Pd(dppf)Cl₂ (38.1 mg, 52.1 umol) and K₂CO₃ (180 mg, 1.30 mmol), purged with N₂ three times. Then the mixture was stirred at 80° C. for 1.5 hours under N₂ atmosphere. On completion, the mixture was concentrated under reduced pressure to give a residue. The crude product was triturated with EtOAc/PE (1:5, 10 mL) at 20° C. for 10 min to give the title compound (280 mg) as a yellow solid. LC-MS (ESI⁺) m/z 515.4 (M+H)⁺.

Step 2—2′-(6′-(Piperazin-1-yl)-[2,3′-bipyridin]-4-yl)-5′,6′-dihydrospiro[cyclobutane-1,7′-pyrrolo[3,2-c]pyridin]-4′(1′H)-one. To a solution of tert-butyl 4-(4-(4′-oxo-1′,4′,5′,6′-tetrahydrospiro[cyclobutane-1,7′-pyrrolo[3,2-c]pyridin]-2′-yl)-[2,3′-bipyridin]-6′-yl)piperazine-1-carboxylate (280 mg, 544 umol) in DCM (5 mL) was added HCl/EtOAc (4 M, 1.40 mL). The mixture was stirred at 20° C. for 0.5 hr. On completion, the mixture was concentrated under reduced pressure to give the title compound (280 mg, HCl) as a brown solid. LC-MS (ESI⁺) m/z 415.2 (M+H)⁺.

### 7-(benzofuran-2-yl)-N-(6-nitropyridin-2-yl)-1H-indazol-5-amine (Intermediate CQ)

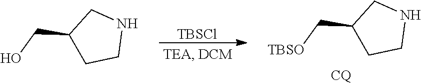

To a solution of [(3R)-pyrrolidin-3-yl]methanol (5 g, 50 mmol) in DCM (50 mL) was added TBSCl (8.20 g, 54.4 mmol, 6.66 mL) and TEA (15.0 g, 148 mmol, 20.7 mL). The mixture was stirred at 25° C. for 12 hrs. On completion, the mixture was filtered and concentrated under reduced pressure to give a residue. The residue was purified by column chromatography (SiO₂, Petroleum ether/Ethyl acetate=1/0, DCM:MeOH=4:1) to give the tittle compound (7 g, 65% yield) as a colorless transparent oil. ¹H NMR (400 MHz, DMSO-d₆) δ=3.57-3.41 (m, 3H), 2.86-2.62 (m, 4H), 2.16 (td, J=6.8, 14.0 Hz, 1H), 1.43-1.20 (m, 2H), 0.07 (s, 6H), 0.00 (s, 9H).

### ((3R)-1-(1-(1-(4-methoxybenzyl)-2,6-dioxopiperidin-3-yl)-3-methyl-2-oxo-2,3-dihydro-1H-benzo[d]imidazol-5-yl)pyrrolidin-3-yl)methyl 4-methylbenzenesulfonate (Intermediate CR)

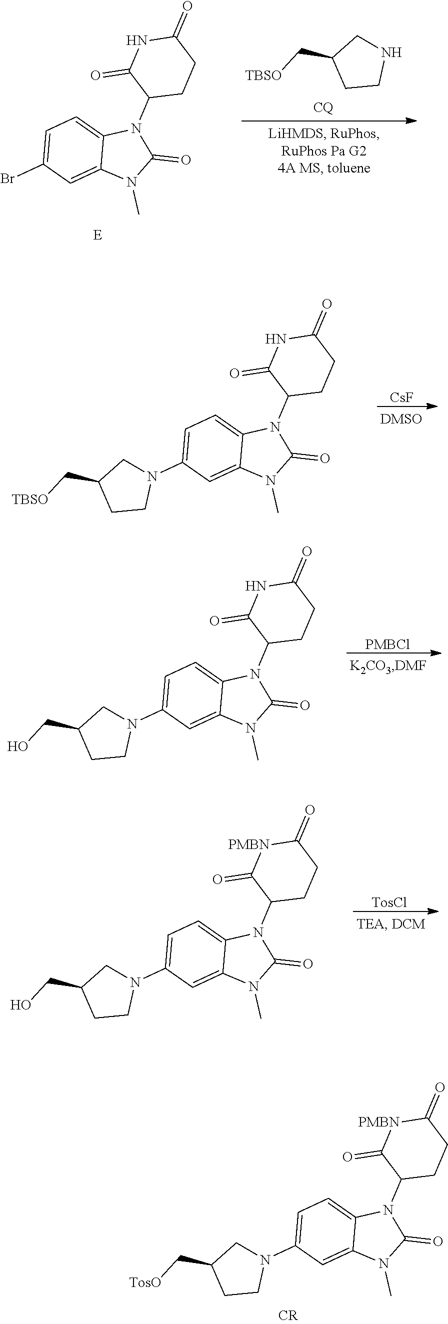

Step 1—3-(5-((R)-3-(((tert-butyldimethylsilyl)oxy)methyl)pyrrolidin-1-yl)-3-methyl-2-oxo-2,3-dihydro-1H-benzo[d]imidazol-1-yl)piperidine-2,6-dione. A mixture of 3-(5-bromo-3-methyl-2-oxo-benzimidazol-1-yl)piperidine-2,6-dione (2 g, 5.91 mmol, Intermediate E), tert-butyl-dimethyl-[[(3R)-pyrrolidin-3-yl]methoxy]silane (1.91 g, 8.87 mmol, Intermediate CQ), 4 Å molecular sieves (2 g) in toluene (50 mL), then the mixture was stirred at 0° C. for 15 min. Next, LiHMDS (1 M, 29.6 mL) was added under N₂ atmosphere and then was added RuPhos (276 mg, 591 umol) [2-(2-aminophenyl)phenyl]-chloro-palladium;dicyclohexyl-[2-(2,6-diisopropoxyphenyl)phenyl]phosphane (460 mg, 591 umol) was added and the mixture was degassed and purged with N₂ three times. Then the mixture was stirred at 100° C. for 1 hr under N₂ atmosphere. On completion, the reaction mixture was filtered and concentrated under reduced pressure to give a residue. The residue was purified by column chromatography (SiO₂, Petroleum ether/Ethyl acetate=10/1 to 1/2) to give the title compound (2 g, 53% yield) as a brown solid. LC-MS (ESI⁺) m/z 473.2 (M+H)⁺.

Step 2—3-(5-((R)-3-(hydroxymethyl)pyrrolidin-1-yl)-3-methyl-2-oxo-2,3-dihydro-1H-benzo[d]imidazol-1-yl)piperidine-2,6-dione. To a solution of 3-[5-[(3R)-3-[[tert-butyl(dimethyl)silyl]oxymethyl]pyrrolidin-1-yl]-3-methyl-2-oxo-benzimidazol-1-yl]piperidine-2,6-dione (1 g, 2 mmol) in DMSO (10 mL) was added CsF (643 mg, 4.23 mmol, 156 uL). The mixture was stirred at 50° C. for 1 hr. On completion, the reaction mixture was filtered and concentrated under reduced pressure to give a residue. The filtrate was purified by reversed-phase HPLC (0.1% FA condition) to give the tittle compound (500 mg, 62% yield) as a brown solid. LC-MS (ESI⁺) m/z 359.2 (M+H)⁺.

Step 3—3-(5-((R)-3-(hydroxymethyl)pyrrolidin-1-yl)-3-methyl-2-oxo-2,3-dihydro-1H-benzo[d]imidazol-1-yl)-1-(4-methoxybenzyl)piperidine-2,6-dione. To a solution of 3-[5-[(3R)-3-(hydroxymethyl)pyrrolidin-1-yl]-3-methyl-2-oxo-benzimidazol-1-yl]piperidine-2,6-dione (400 mg, 1.12 mmol) in DMF (10 mL) was added K₂CO₃ (309 mg, 2.23 mmol), then was added PMB-Cl (227 mg, 1.45 mmol, 198 uL) at 0° C. over 2 min. The mixture was then stirred at 20° C. for 12 hrs. On completion, the reaction mixture was partitioned between H₂O (30 mL) and ethyl acetate (50 mL). The organic phase was separated, washed with (30 mL×3), dried over Na₂SO₄, filtered and concentrated under reduced pressure to give a residue. The residue was purified by column chromatography (SiO₂, Petroleum ether/Ethyl acetate=20/1 to 0/1) to give the title compound (320 mg, 51% yield) as a white solid. LC-MS (ESI+) m/z 479.3 (M+H)⁺.

Step 4—((3R)-1-(1-(1-(4-methoxybenzyl)-2,6-dioxopiperidin-3-yl)-3-methyl-2-oxo-2,3-dihydro-1H-benzo[d]imidazol-5-yl)pyrrolidin-3-yl)methyl 4-methylbenzenesulfonate. To a solution of 3-[5-[(3R)-3-(hydroxymethyl)pyrrolidin-1-yl]-3-methyl-2-oxo-benzimidazol-1-yl]-1-[(4-methoxyphenyl)methyl]piperidine-2,6-dione (300 mg, 627 umol) in DCM (5 mL) was added TEA (190 mg, 1.88 mmol, 262 uL) and TosCl (359 mg, 1.88 mmol). The mixture was stirred at 25° C. for 12 hrs. On completion, the reaction mixture was filtered and concentrated under reduced pressure to give a residue. The residue was purified by column chromatography (SiO₂, Petroleum ether/Ethyl acetate=2/1 to 0/1) to give the title compound (350 mg, 73% yield) as a white solid. LC-MS (ESI+) m/z 633.2 (M+H)⁺.

### (1-(1-(4-methoxybenzyl)-2,6-dioxopiperidin-3-yl)-3-methyl-2-oxo-2,3-dihydro-1H-benzo[d]imidazol-5-yl)boronic acid (Intermediate CS)

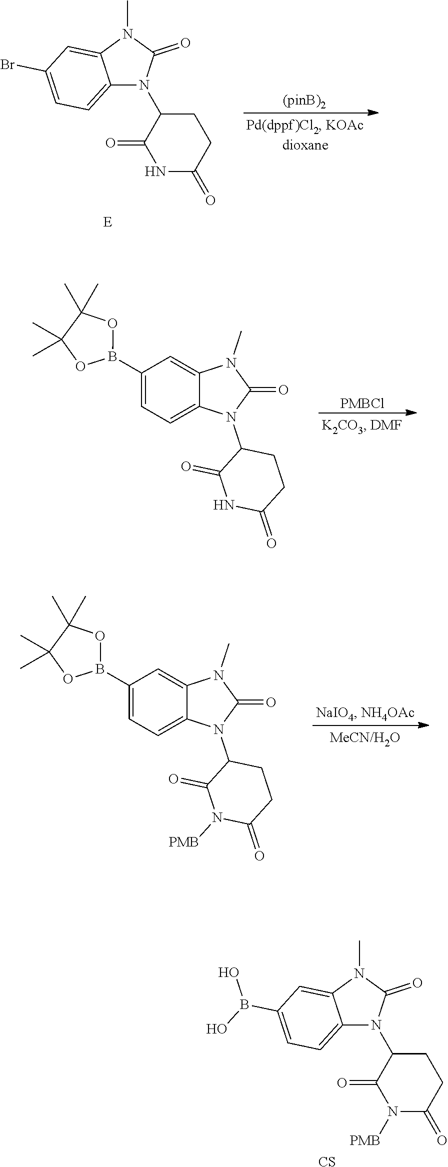

Step 1—3-(3-Methyl-2-oxo-5-(4,4,5,5-tetramethyl-1,3,2-dioxaborolan-2-yl)-2,3-dihydro-1H-benzo[d]imidazol-1-yl)piperidine-2,6-dione. A mixture of 3-(5-bromo-3-methyl-2-oxo-2,3-dihydro-1H-benzo[d]imidazol-1-yl)piperidine-2,6-dione (5 g, 14.8 mmol, Intermediate E), Pin₂B₂(7.51 g, 29.6 mmol), Pd(dppf)Cl₂ (1.08 g, 1.48 mmol), and KOAc (4.35 g, 44.4 mmol) in dioxane (150 mL) was degassed and purged with N₂ three times. Then the mixture was stirred at 80° C. for 2 hrs under N₂ atmosphere. On completion, the reaction mixture was partitioned between EA (150 mL) and H₂O (300 mL). The organic phase was separated, washed with saturated salt solution (100 mL×3), dried over Na₂SO₄, filtered and concentrated under reduced pressure to give the title compound (6.3 g) as a white solid. LC-MS (ESI⁺) m/z 386.2 (M+H)⁺.

Step 2—1-(4-Methoxybenzyl)-3-(3-methyl-2-oxo-5-(4,4,5,5-tetramethyl-1,3,2-dioxaborolan-2-yl)-2,3-dihydro-1H-benzo[d]imidazol-1-yl)piperidine-2,6-dione. To a solution of 3-(3-methyl-2-oxo-5-(4,4,5,5-tetramethyl-1,3,2-dioxaborolan-2-yl)-2,3-dihydro-1H-benzo[d]imidazol-1-yl)piperidine-2,6-dione (3 g, 7.79 mmol) in DMF (30 mL) was added PMB-Cl (1.22 g, 7.79 mmol, 1.06 mL) and K₂CO₃ (2.15 g, 15.6 mmol). The mixture was stirred at 25° C. for 12 hrs. On completion, the reaction mixture was filtered. The filtrate was quenched with H₂O (60 mL) and extracted with EA (50 mL×3). The combined organic layers were washed with brine (50 mL×2), dried over Na₂SO₄, filtered and concentrated under reduced pressure to give a residue. The residue was purified by prep-HPLC (FA condition) to give the compound (2.0 g, 44% yield, FA) as a white solid. LC-MS (ESI+) m/z 506.0 (M+H)⁺.

Step 3—(1-(1-(4-Methoxybenzyl)-2,6-dioxopiperidin-3-yl)-3-methyl-2-oxo-2,3-dihydro-1H-benzo[d]imidazol-5-yl)boronic acid. To a solution of 1-(4-methoxybenzyl)-3-(3-methyl-2-oxo-5-(4,4,5,5-tetramethyl-1,3,2-dioxaborolan-2-yl)-2,3-dihydro-1H-benzo[d]imidazol-1-yl)piperidine-2,6-dione (1.5 g, 2.97 mmol) in MeCN (15 mL) and H₂O (7.5 mL) was added NH₄OAc (457 mg, 5.94 mmol) and NaIO₄ (1.90 g, 8.90 mmol, 493 uL). The mixture was stirred at 25° C. for 12 hrs. On completion, the reaction mixture was concentrated under reduced pressure to remove the solvent. The residue was purified by column chromatography (SiO₂, Petroleum ether/Ethyl acetate=5/1 to 1/1) to give the title COMPOUND (1 g, 76% yield) as a white solid. LC-MS (ESI⁺) m/z 424.3 (M+H)⁺.

### 3-(5-Hydroxy-3-methyl-2-oxo-2,3-dihydro-1H-benzo[d]imidazol-1-yl)-1-(4-methoxybenzyl)piperidine-2,6-dione (Intermediate CT)

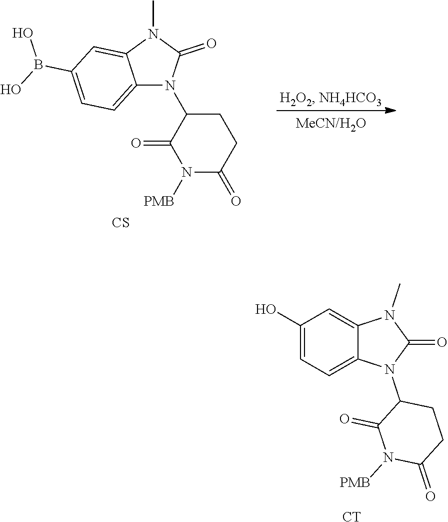

To a solution of (1-(1-(4-methoxybenzyl)-2,6-dioxopiperidin-3-yl)-3-methyl-2-oxo-2,3-dihydro-1H-benzo[d]imidazol-5-yl)boronic acid (800 mg, 1.89 mmol, Intermediate CS) in MeCN (10 mL) and H₂O (5 mL) was added NH₄HCO₃ (149 mg, 1.89 mmol, 155 uL) and H₂O₂(429 mg, 3.78 mmol, 363 uL, 30% solution). The mixture was stirred at 20° C. for 2 hrs. On completion, the mixture was quenched with NaHSO₃ solution (20 mL), then extracted with EtOAc (100 mL×3). The organic layer was then concentrated under reduced pressure to give the title COMPOUND (690 mg) as a pink solid. LC-MS (ESI⁺) m/z 396.0 (M+H)⁺.

### 2-((1-(1-(4-methoxybenzyl)-2,6-dioxopiperidin-3-yl)-3-methyl-2-oxo-2,3-dihydro-1H-benzo[d]imidazol-5-yl)oxy)acetaldehyde (Intermediate CU)

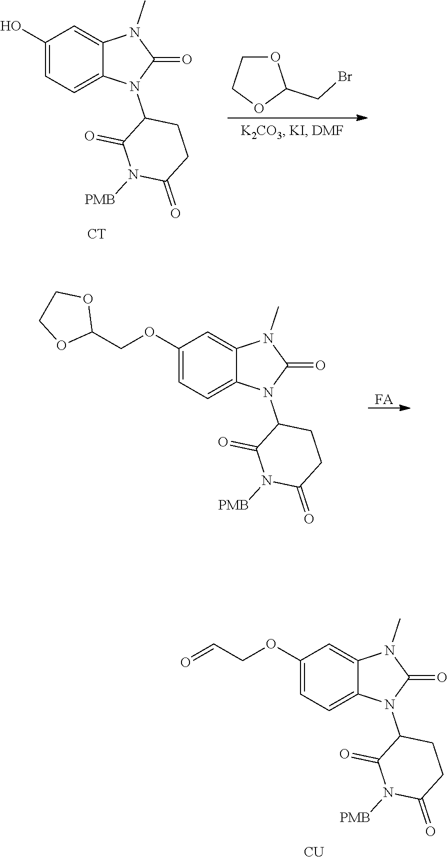

Step 1—3-(5-((1,3-Dioxolan-2-yl)methoxy)-3-methyl-2-oxo-2,3-dihydro-1H-benzo[d]imidazol-1-yl)-1-(4-methoxybenzyl)piperidine-2,6-dione. To a solution of 3-(5-hydroxy-3-methyl-2-oxo-2, 3-dihydro-1H-benzo[d]imidazol-1-yl)-1-(4-methoxybenzyl) piperidine-2, 6-dione (45.6 mg, 273 umol, 27.9 uL, Intermediate CT) in DMF (1 mL) was added K₂CO₃ (62.9 mg, 455 umol) and KI (37.8 mg, 227 umol). The mixture was stirred at 100° C. for 2 hrs. On completion, the reaction mixture was partitioned between EA (3 mL) and H₂O (3 mL). The organic phase was washed with brine (1 mL×3), dried over Na₂SO₄, filtered and concentrated under reduced pressure to give a residue. The residue was purified by column chromatography (SiO₂, Petroleum ether/Ethyl acetate=1/0 to 1/1) to give the title COMPOUND (21 mg, 14% yield) as a white solid. LC-MS (ESI⁺) m/z 482.2 (M+H)⁺.

Step 2—2-((1-(1-(4-Methoxybenzyl)-2,6-dioxopiperidin-3-yl)-3-methyl-2-oxo-2,3-dihydro-1H-benzo[d]imidazol-5-yl)oxy)acetaldehyde. 3-(5-((1,3-dioxolan-2-yl)methoxy)-3-methyl-2-oxo-2,3-dihydro-1H-benzo[d]imidazol-1-yl)-1-(4-methoxybenzyl)piperidine-2,6-dione (21 mg, 37.5 umol) was added to FA (1 mL). The mixture was then stirred at 25° C. for 1.5 hrs. On completion, the reaction mixture was concentrated under reduced pressure to give the title COMPOUND (16 mg) as a white solid. LC-MS (ESI⁺) m/z 456.1 (M+H)⁺.

### ((3-bromocyclobutyl)methoxy)(tert-butyl)dimethylsilane (Intermediate CV)

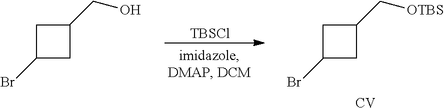

To a solution of (3-bromocyclobutyl)methanol (3.5 g, 21.2 mmol, CAS #1896670-35-1) in DCM (100 mL) was added imidazole (2.89 g, 42.4 mmol), DMAP (259 mg, 2.12 mmol) and TBSCl (4.79 g, 31.8 mmol). Then the mixture was stirred at 25° C. for 1 hr. On completion, the reaction mixture was concentrated under reduced pressure to give a residue. The residue was purified by or by prep-TLC (SiO₂, Petroleum ether/Ethyl acetate=0/1) to give a compound (4.9 g, 82% yield) as a colorless oil. ¹H NMR (400 MHz, CDCl₃-d) δ=4.50 (quin, J=7.2 Hz, 1H), 3.53 (d, J=5.2 Hz, 2H), 2.70-2.58 (m, 1H), 2.53-2.46 (m, 4H), 0.85 (s, 9H), 0.00 (s, 6H).

### 3-(5-(3-(hydroxymethyl)cyclobutyl)-3-methyl-2-oxo-2,3-dihydro-1H-benzo[d]imidazol-1-yl)-1-(4-methoxybenzyl)piperidine-2,6-dione (Intermediate CW)

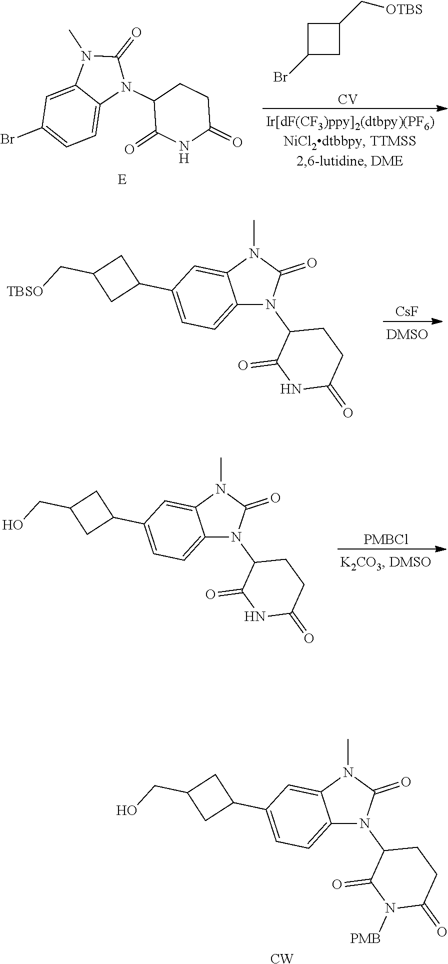

Step 1—3-(5-(3-(((Tert-butyldimethylsilyl)oxy)methyl)cyclobutyl)-3-methyl-2-oxo-2,3-dihydro-1H-benzo[d]imidazol-1-yl)piperidine-2,6-dione. To an 40 mL vial equipped with a stir bar was added 3-(5-bromo-3-methyl-2-oxo-2,3-dihydro-1H-benzo[d]imidazol-1-yl)piperidine-2,6-dione (2 g, 5.91 mmol, Intermediate E) ((3-bromocyclobutyl)methoxy)(tert-butyl)dimethylsilane (2.15 g, 7.69 mmol, Intermediate CV), Ir[dF(CF₃)ppy]₂(dtbpy)(PF₆) (66.4 mg, 59.1 umol), NiCl₂.dtbbpy (35.3 mg, 88.7 umol), TTMSS (1.47 g, 5.91 mmol, 1.82 mL) and 2,6-lutidine (1.27 g, 11.8 mmol, 1.38 mL) in DME (32 mL). The reaction was stirred and irradiated with a 10 W blue LED lamp (3 cm away), with cooling water to keep the reaction temperature at 25° C. for 14 hrs. On completion, the reaction mixture was concentrated under reduced pressure to give a residue. The residue was purified by prep-HPLC (0.1% FA) to give the title compound (1.1 g, 14% yield, FA) as a yellow solid. LC-MS (ESI+) m/z 458.3 (M+H)⁺.

Step 2—3-(5-(3-(Hydroxymethyl)cyclobutyl)-3-methyl-2-oxo-2,3-dihydro-1H-benzo[d]imidazol-1-yl)piperidine-2,6-dione. To a solution of 3-(5-(3-(((tert-butyldimethylsilyl)oxy)methyl)cyclobutyl)-3-methyl-2-oxo-2,3-dihydro-1H-benzo[d]imidazol-1-yl)piperidine-2,6-dione (1 g, 2.19 mmol) in DMSO (20 mL) was added CsF (996 mg, 6.56 mmol). The mixture was stirred at 50° C. for 1 hr. On completion, the reaction mixture was filtered and quenched with of H₂O (40 mL) and then extracted with EtOAc (50 mL×3). The combined organic layers were washed with brine (50 mL×2), dried over Na₂SO₄, filtered and concentrated under reduced pressure to give the title compound (800 mg) as a yellow solid. LC-MS (ESI⁺) m/z 344.2. (M+H)⁺.

Step 3—3-(5-(3-(Hydroxymethyl)cyclobutyl)-3-methyl-2-oxo-2,3-dihydro-1H-benzo[d]imidazol-1-yl)-1-(4-methoxybenzyl)piperidine-2,6-dione. To a solution of 3-(5-(3-(hydroxymethyl)cyclobutyl)-3-methyl-2-oxo-2,3-dihydro-1H-benzo[d]imidazol-1-yl)piperidine-2,6-dione (750 mg, 2.18 mmol) in DMSO (10 mL) was added K₂CO₃ (604 mg, 4.37 mmol) and PMB-Cl (342 mg, 2.18 mmol). The mixture was stirred at 25° C. for 2 hrs. On completion, the reaction mixture was concentrated under reduced pressure to give a residue. The residue was purified by prep-HPLC (0.1% FA) to give the title compound (750 mg, 64% yield, FA) as a white solid. LC-MS (ESI+) m/z 464.2 (M+H)⁺.

### 3-(5-((1R,3R)-3-(hydroxymethyl)cyclobutyl)-3-methyl-2-oxo-2,3-dihydro-1H-benzo[d]imidazol-1-yl)-1-(4-methoxybenzyl)piperidine-2,6-dione (Intermediate CX) and 3-(5-((1s,3s)-3-(hydroxymethyl)cyclobutyl)-3-methyl-2-oxo-2,3-dihydro-1H-benzol[d]imidazol-1-yl)-1-(4-methoxybenzyl)piperidine-2,6-dione (Intermediate CY)

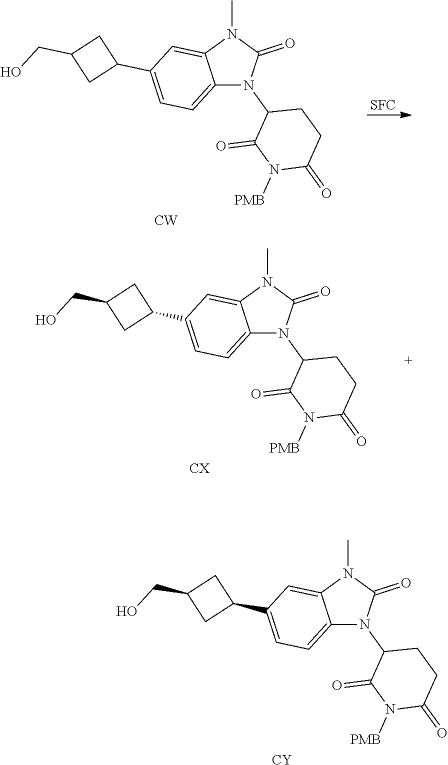

3-(5-(3-(hydroxymethyl)cyclobutyl)-3-methyl-2-oxo-2,3-dihydro-1H-benzo[d]imidazol-1-yl)-1-(4-methoxybenzyl)piperidine-2,6-dione (518 mg, 1.12 mmol, Intermediate CW) was separated by SFC (column: DAICEL CHIRALPAK AD (250 mm×30 mm×10 um); mobile phase: [Neu-EtOH]; B %: 45%-45%, 4.2; 80 min) to obtain 3-(5-((1r,3r)-3-(hydroxymethyl)cyclobutyl)-3-methyl-2-oxo-2,3-dihydro-1H-benzo[d]imidazol-1-yl)-1-(4-methoxybenzyl)piperidine-2,6-dione as a white solid (300 mg, 57% yield, LC-MS (ESI⁺) m/z 464.4 (M+H)+) and 3-(5-((1s,3s)-3-(hydroxymethyl)cyclobutyl)-3-methyl-2-oxo-2,3-dihydro-1H-benzo[d]imidazol-1-yl)-1-(4-methoxybenzyl)piperidine-2,6-dione as a white solid(200 mg, 38% yield, LC-MS (ESI+) m/z 464.4 (M+H)+).

### ((1R,3R)-3-(1-(1-(4-methoxybenzyl)-2,6-dioxopiperidin-3-yl)-3-methyl-2-oxo-2,3-dihydro-1H-benzo[d]imidazol-5-yl)cyclobutyl)methyl methanesulfonate (Intermediate CZ)

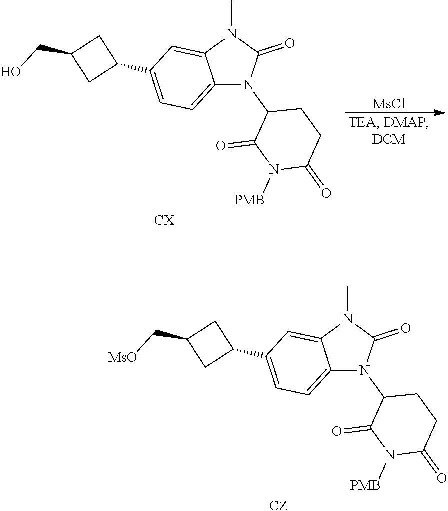

To a solution of 3-(5-((1r,3r)-3-(hydroxymethyl)cyclobutyl)-3-methyl-2-oxo-2,3-dihydro-1H-benzo[d]imidazol-1-yl)-1-(4-methoxybenzyl)piperidine-2,6-dione (300 mg, 647 umol, Intermediate CX) in DCM (4 mL) was added TEA (131 mg, 1.29 mmol), DMAP (7.91 mg, 64.7 umol) and MsCl (1.48 g, 12.9 mmol) at 0° C., then the reaction was stirred at 25° C. for 12 hrs. On completion, added aqueous sodium hydrogencarbonate solution (2 mL) at 0° C. until pH was 8.0 and extracted with DCM (10 mL). The combined organic layers were dried over Na₂SO₄, filtered and concentrated under reduced pressure to give a residue. The residue was purified by column chromatography (SiO₂, DCM/MeOH=10/1) to give the title compound (350 mg, 90% yield) as a white solid. LC-MS (ESI⁺) m/z 542.1 (M+H)⁺.

### ((1S,3S)-3-(1-(1-(4-methoxybenzyl)-2,6-dioxopiperidin-3-yl)-3-methyl-2-oxo-2,3-dihydro-1H-benzo[d]imidazol-5-yl)cyclobutyl)methyl methanesulfonate (Intermediate DA)

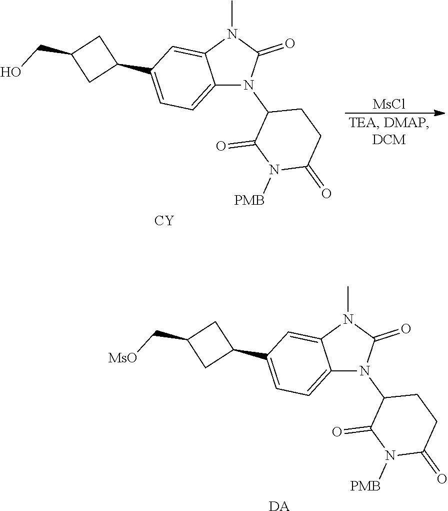

To a solution of 3-(5-((1s,3s)-3-(hydroxymethyl)cyclobutyl)-3-methyl-2-oxo-2,3-dihydro-1H-benzo[d]imidazol-1-yl)-1-(4-methoxybenzyl)piperidine-2,6-dione (200 mg, 431 umol, Intermediate CY) in DCM (4 mL) was added TEA (87.3 mg, 863 umol), DMAP (5.27 mg, 43.1 umol) and MsCl (988 mg, 8.63 mmol) at 0° C. Then the reaction was stirred at 25° C. for 12 hrs. On completion, added aqueous sodium hydrogencarbonate solution (2 mL) at 0° C. until the pH=8 and then the mixture was extracted with DCM (10 mL). The combined organic layers were dried over Na₂SO₄, filtered and the filtrate was concentrated under reduced pressure to give a residue. The residue was purified by column chromatography (SiO₂, DCM/MeOH=10/1) to give the title compound (250 mg, 99% yield) as a white solid. LC-MS (ESI⁺) m/z 542.1 (M+H)⁺.

### Tert-butyl 4-(3-isopropoxy-5-(4,4,5,5-tetramethyl-1,3,2-dioxaborolan-2-yl)phenyl)piperazine-1-carboxylate (Intermediate DB)

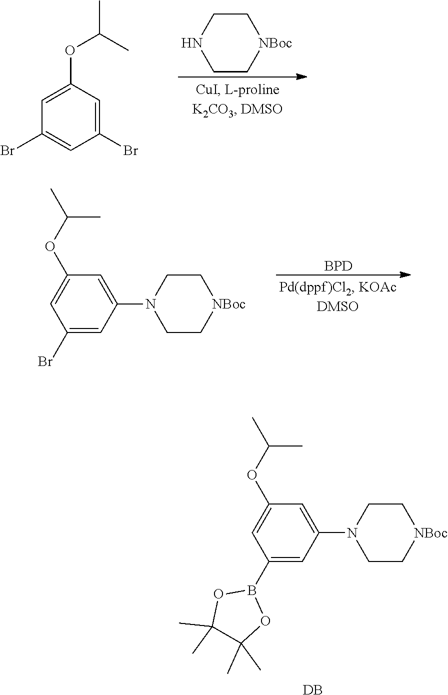

Step 1—Tert-butyl 4-(3-bromo-5-isopropoxyphenyl)piperazine-1-carboxylate. To a solution of 1,3-dibromo-5-isopropoxybenzene (2.0 g, 6.80 mmol, CAS #1112210-82-8) and tert-butyl piperazine-1-carboxylate (1.27 g, 6.80 mmol, CAS #143238-38-4) in DMSO (20 mL) was added CuI (1.94 g, 0.01 mol), L-proline (0.16 mg, 1.36 mmol) and K₂CO₃ (1.88 g, 13.6 mmol). The mixture was degassed and purged with N₂ three times. Then the mixture was stirred at 80° C. for 12 hrs under N₂ atmosphere. The reaction mixture was quenched with water (20 mL), and then diluted with ethyl acetate (30 mL) and extracted with ethyl acetate (30 mL×3). The combined organic layers were washed with sat. brine (50 mL×3), dried over Na₂SO₄, filtered and concentrated under reduced pressure to give a residue. The residue was purified by column chromatography (SiO₂, Petroleum ether/Ethyl acetate=20/1 to 3/1) to give the title compound (0.42 g, 15% yield) as a yellow oil. LC-MS (ESI⁺) m/z 400.6 (M+H)⁺.

Step 2—Tert-butyl 4-(3-isopropoxy-5-(4,4,5,5-tetramethyl-1,3,2-dioxaborolan-2-yl)phenyl)piperazine-1-carboxylate. To a solution of tert-butyl 4-(3-bromo-5-isopropoxyphenyl)piperazine-1-carboxylate (0.366 g, 0.916 mmol) and BPD (0.698 g, 2.75 mmol) in DMSO (8 mL) was added KOAc (0.269 g, 2.75 mmol) and Pd(dppf)Cl₂ (0.067 g, 0.092 mmol). The mixture was degassed and purged with N₂ three times. Then the mixture was stirred at 80° C. for 3 hrs under N₂ atmosphere. The reaction mixture was quenched with water (10 mL), and then diluted with ethyl acetate (20 mL) and extracted with ethyl acetate (20 mL×3). The combined organic layers were washed with sat. brine (30 mL×3), dried over Na₂SO₄, filtered and concentrated under reduced pressure to give a residue. The residue was purified by column chromatography (SiO₂, Petroleum ether/Ethyl acetate=1:0 to 10:1) to give the title compound (0.128 g, 30% yield) as a yellow solid. LC-MS (ESI⁺) m/z 447.0 (M+H)⁺.

### (R)-3-(3-isopropoxy-5-(piperazin-1-yl)phenyl)-10-methyl-9,10,11,12-tetrahydro-8H-[1,4]diazepino[5′,6′:4,5]thieno[3,2-f]quinolin-8-one (Intermediate DC)

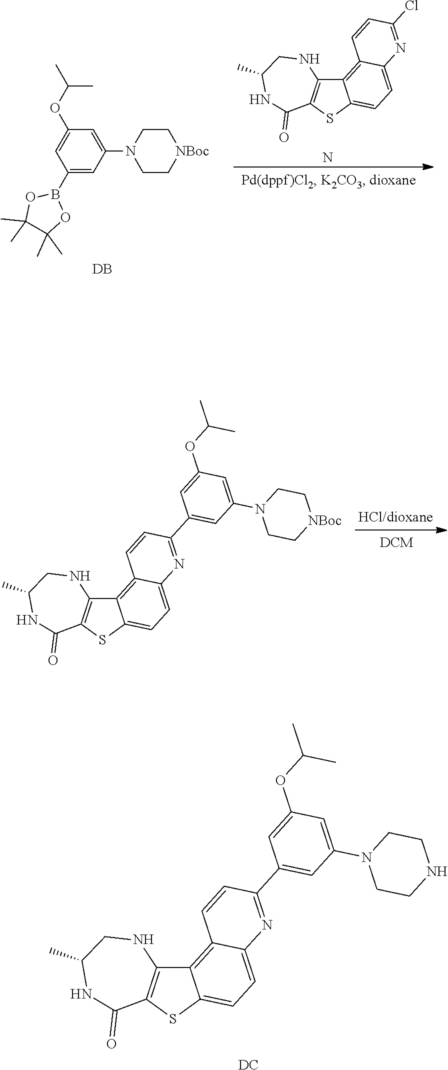

Step 1—((R)-tert-butyl 4-(3-isopropoxy-5-(10-methyl-8-oxo-9,10,11,12-tetrahydro-8H-diazepino[5′,6′:4,5]thieno[3,2-f]quinolin-3-yl)phenyl)piperazine-1-carboxylate. To a solution of tert-butyl 4-(3-isopropoxy-5-(4,4,5,5-tetramethyl-1,3,2-dioxaborolan-2-yl)phenyl)piperazine-1-carboxylate (128 mg, 0.286 mmol, Intermediate DB) and (R)-3-chloro-10-methyl-9,10,11,12-tetrahydro-8H-[1,4]diazepino[5′,6′:4,5]thieno[3,2-f]quinolin-8-one (46.3 mg, 0.143 mmol, Intermediate N) in dioxane (6.4 mL) and H₂O (1.28 mL) was added Pd(dppf)Cl₂ (10.2 mg, 0.014 mmol) and K₂CO₃ (59.0 mg, 0.430 mmol). The mixture was degassed and purged with N₂ three times. Then the mixture was stirred at 80° C. for 12 hrs under N₂ atmosphere. The reaction mixture was quenched with water (10 mL), and then diluted with ethyl acetate (20 mL) and extracted with ethyl acetate (20 mL×3). The combined organic layers were washed with sat. brine (30 mL×3), dried over Na₂SO₄, filtered and concentrated under reduced pressure to give a residue. The residue was dissolved with ethyl acetate, and then precipitated with petroleum ether (10 ml) and washed with the mixture solution (petroleum ether:ethyl acetate=1:1, 15 mL), filtered and concentrated under reduced pressure to give the title compound (85 mg) as a black solid. LC-MS (ESI⁺) m/z 602.2 (M+H)⁺.

Step 2—(R)-3-(3-isopropoxy-5-(piperazin-1-yl)phenyl)-10-methyl-9,10,11,12-tetrahydro-8H-[1,4]diazepino[5′,6′:4,5]thieno[3,2-f]quinolin-8-one. To a solution of (R)-tert-butyl 4-(3-isopropoxy-5-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (75 mg, 0.125 mmol) in DCM (1 mL) was added HCl/dioxane (4 M, 1 mL). The mixture was degassed and purged with N₂ three times. Then the mixture was stirred at 25° C. for 10 min. The reaction mixture was concentrated under reduced pressure to give a residue. The residue was purified by prep-HPLC (column: Phenomenex C18 150×25 mm×10 um; mobile phase: [water (NH4HCO₃)-ACN]; B %: 28%-58%, 8 min) to give the title compound (2.8 mg, 4% yield) as a yellow solid. LC-MS (ESI⁺) m/z 502.2 (M+H)⁺. 1H NMR (400 MHz, DMSO-d6) δ=9.20 (d, J=8.8 Hz, 1H), 8.21 (d, J=9.2 Hz, 1H), 8.12 (d, J=9.2 Hz, 1H), 8.09-8.06 (m, 1H), 8.04-7.99 (m, 1H), 7.42 (s, 1H), 7.26 (s, 1H), 7.18-7.10 (m, 1H), 6.57 (s, 1H), 4.78-4.74 (m, 1H), 3.80-3.60 (m, 1H), 3.50-3.49 (m, 4H), 3.20-3.17 (m, 4H), 2.92 (s, 2H), 1.32 (d, J=5.6 Hz, 6H), 1.20-1.10 (d, J=7.2 Hz, 3H).

### Tert-butyl 4-(3-isopropyl-5-(4,4,5,5-tetramethyl-1,3,2-dioxaborolan-2-yl)phenyl)piperazine-1-carboxylate (Intermediate DD)

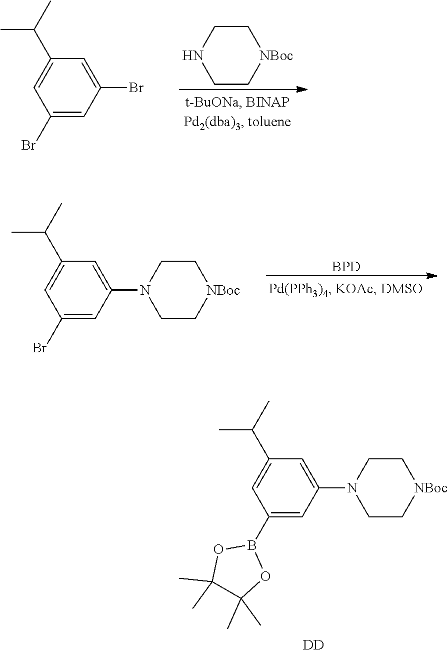

Step 1—Tert-butyl 4-(3-bromo-5-isopropylphenyl)piperazine-1-carboxylate. To a solution of tert-butyl piperazine-1-carboxylate (3.0 g, 16 mmol, CAS #143238-38-4) and 1,3-dibromo-5-isopropylbenzene (4.0 g, 14 mmol, CAS #6 2655-20-3) in toluene (50 mL) was added t-BuONa (2.8 g, 29 mmol), BINAP (0.9 g, 1.4 mmol) and Pd₂(dba)₃ (0.7 g, 0.7 mmol), then the mixture was stirred at 110° C. for 4.0 hrs. The reaction mixture was quenched with NH₄Cl 20 mL at 25° C., and then diluted with H₂O (20 mL) and extracted with ethyl acetate (20 mL×2). The combined organic layers were washed with sat. brine (20 mL), dried over Na₂SO₄, filtered and concentrated under reduced pressure to give a residue. The residue was purified by column chromatography (SiO₂, Petroleum ether/Ethyl acetate=1:0 to 100:1) a to give the title compound (4.2 g, 68% yield) as a yellow oil. LC-MS (ESI⁺) m/z 383.2 (M+1); ¹H NMR (DMSO-d₆, 400 MHz) δ 6.88 (t, 1H, J=1.6 Hz), 6.81 (d, 2H, J=8.4 Hz), 3.50-3.40 (m, 4H), 3.20-3.10 (m, 4H), 2.90-2.70 (m, 1H), 1.42 (s, 9H), 1.16 (d, 6H, J=7.0 Hz).

Step 2—Tert-butyl 4-(3-isopropyl-5-(4,4,5,5-tetramethyl-1,3,2-dioxaborolan-2-yl)phenyl)piperazine-1-carboxylate. To a solution of tert-butyl 4-(3-bromo-5-isopropylphenyl)piperazine-1-carboxylate (3.7 g, 9.8 mmol) and BPD (7.4 g, 29 mmol, CAS #73183-34-3) in DMSO (50.0 mL) was added KOAc (2.9 g, 29.0 mmol) and Pd(PPh₃)₄(1.1 g, 1.0 mmol), then the mixture was stirred at 80° C. for 7 hrs. On completion, the reaction mixture was quenched with H₂O (20 mL) at 25° C., and then diluted with ethyl acetate (20 mL) and extracted with ethyl acetate (20 mL×2). The combined organic layers were washed with sat. brine (20 mL), dried over Na₂SO₄, filtered and concentrated under reduced pressure to give a residue. The residue was purified by column chromatography (SiO₂, Petroleum ether/Ethyl acetate=20/1 to 5/1) to give the title compound (2.2 g, 45% yield) as a white solid. LC-MS (ESI⁺) m/z 431.4 (M+1). ¹H NMR (DMSO-d₆, 400 MHz) δ 7.00-6.90 (m, 3H), 3.45 (s, 4H), 3.10-3.00 (m, 4H), 2.90-2.80 (m, 1H), 1.42 (s, 9H), 1.28 (s, 12H), 1.18 (s, 6H).

### (R)-3-(3-isopropyl-5-(piperazin-1-yl)phenyl)-10-methyl-9,10,11,12-tetrahydro-8H-[1,4]diazepino[5′,6′:4,5]thieno[3,2-f]quinolin-8-one (Intermediate DE)

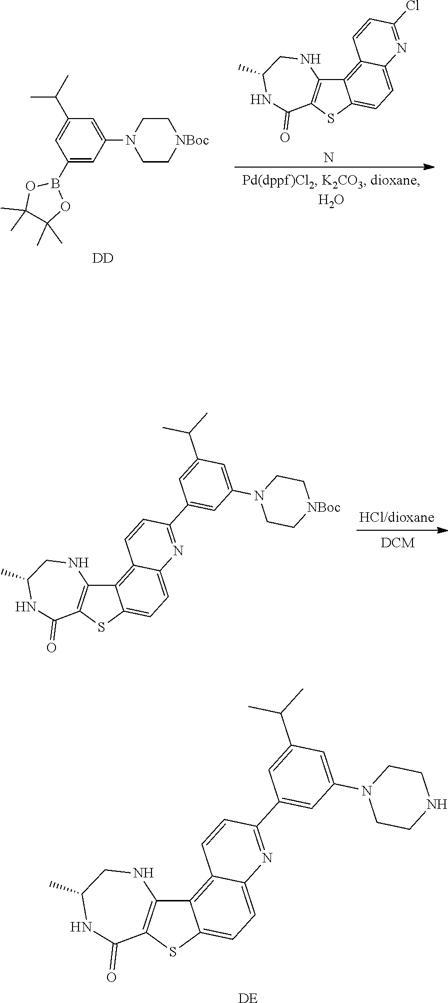

Step 1—(R)-tert-butyl 4-(3-isopropyl-5-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. To a solution of tert-butyl 4-(3-isopropyl-5-(4,4,5,5-tetramethyl-1,3,2-dioxaborolan-2-yl)phenyl) piperazine-1-carboxylate (0.6 g, 1.4 mmol, Intermediate DD) and (R)-3-chloro-10-methyl-9,10,11,12-tetrahydro-8H-[1,4]diazepino[5′,6′:4,5]thieno[3,2-f]quinolin-8-one (0.3 g, 0.9 mmol, Intermediate N) in dioxane (10 mL) and H₂O (1.0 mL) was added K₂CO₃ (0.4 g, 2.7 mmol) and Pd(dppf)Cl₂ (99 mg, 0.1 mmol), then the mixture was stirred at 100° C. for 16 hrs. The reaction mixture was quenched with H₂O (20 mL) at 25° C., and then diluted with ethyl acetate (20 mL) and extracted with ethyl acetate (20 mL×2). The combined organic layers were washed with sat. brine (20 mL), dried over Na₂SO₄, filtered and concentrated under reduced pressure to give a residue. The residue was purified by column chromatography (SiO₂, Petroleum ether/Ethyl acetate=20/1 to 0/1) to give the title compound (0.4 g, 53% yield) as a yellow solid. LC-MS (ESI⁺) m/z 586.3 (M+1)⁺.

Step 2—(R)-3-(3-isopropyl-5-(piperazin-1-yl)phenyl)-10-methyl-9,10,11,12-tetrahydro-8H-[1,4]diazepino[5′,6′:4,5]thieno[3,2-f]quinolin-8-one. To a solution of (R)-tert-butyl 4-(3-isopropyl-5-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (0.4 g, 0.7 mmol) in DCM (2.5 mL) was added HCl/dioxane (4M, 5 mL), then the mixture was stirred at 20° C. for 2.0 hrs. The reaction mixture was concentrated under reduced pressure to give the title compound (469 mg, HCl) as a reddish brown solid without purification. LC-MS (ESI⁺) m/z 486.2 (M+1)⁺.

### 3-(3-methyl-2-oxo-5-(piperazin-1-yl)-2,3-dihydro-1H-benzo[d]imidazol-1-yl)piperidine-2,6-dione (Intermediate DF)

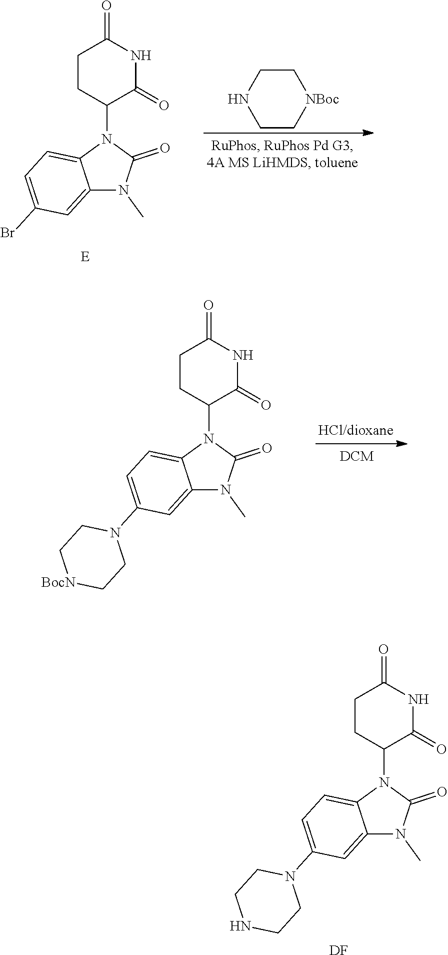 [SMILES: Cn1c(=O)n(C2CCC(=O)NC2=O)c2ccc(Br)cc21 ; CC(C)(C)OC(=O)N1CCNCC1 ; Cn1c(=O)n(C2CCC(=O)NC2=O)c2ccc(N3CCN(C(=O)OC(C)(C)C)CC3)cc21 ; Cn1c(=O)n(C2CCC(=O)NC2=O)c2ccc(N3CCNCC3)cc21 ; "DF"]

Step 1—Tert-butyl 4-(1-(2,6-dioxopiperidin-3-yl)-3-methyl-2-oxo-2,3-dihydro-1H-benzo[d]imidazol-5-yl)piperazine-1-carboxylate. To a solution of 3-(5-bromo-3-methyl-2-oxo-benzimidazol-1-yl)piperidine-2,6-dione (3 g, 8.87 mmol, Intermediate E) and tert-butyl piperazine-1-carboxylate (1.65 g, 8.87 mmol) in toluene (80 mL) was added 4 Å molecular sieves (5 g), RuPhos (207 mg, 444 umol) and RuPhos Pd G3 (371 mg, 444 umol). Then LiHMDS (1 M, 53.2 mL) was added at 0° C. Then the mixture was stirred at 100° C. for 12 hrs. The reaction mixture was diluted with H₂O (30 mL) and extracted with EA (30 mL×5). The combined organic layers were dried over Na₂SO₄, filtered and concentrated under reduced pressure to give a residue. The residue was purified by column chromatography (SiO₂, Petroleum ether/Ethyl acetate=1/0 to 0/1) to give the title compound (300 mg, 5% yield) as a white solid. LC-MS (ESI⁺) m/z 443.7 (M+1)⁺.

Step 2—3-(3-methyl-2-oxo-5-(piperazin-1-yl)-2,3-dihydro-1H-benzo[d]imidazol-1-yl)piperidine-2,6-dione. To a solution of tert-butyl 4-[1-(2,6-dioxo-3-piperidyl)-3-methyl-2-oxo-benzimidazol-5-yl]piperazine-1-carboxylate (200 mg, 451 umol) in DCM (4 mL) was added HCl/dioxane (4 M, 1.69 mL). The mixture was stirred at 25° C. for 2 hrs. On completion, the reaction mixture was concentrated under reduced pressure to give the title compound (200 mg, HCl) as a yellow solid. LC-MS (ESI⁺) m/z 344.0 (M+1)⁺.

### (R)-methyl 1-((2-aminobutyl)amino)thieno[3,2-f]quinoline-2-carboxylate (Intermediate DG)

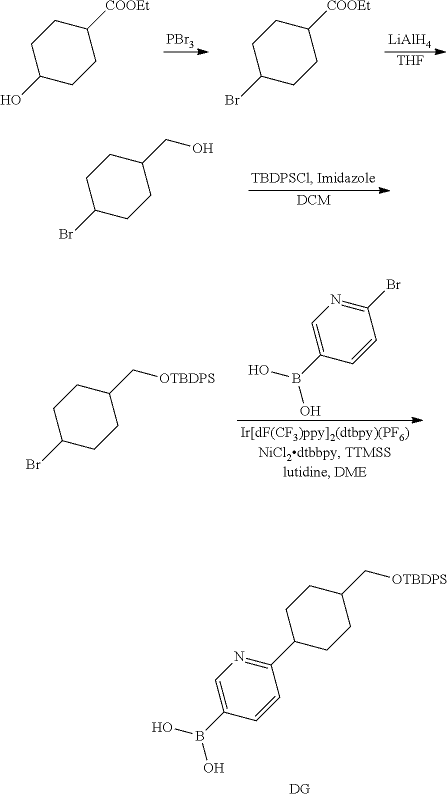

Step 1—Ethyl 4-bromocyclohexanecarboxylate. Ethyl 4-hydroxycyclohexanecarboxylate (10 g, 58 mmol) was added to PBr₃ (5.5 g, 20.3 mmol, CAS #17159-80-7) and the mixture was stirred at 80° C. for 6 hrs. On completion, the mixture was quenched with sat. NaHCO₃ until the pH=8, then extracted with EtOAc (100 mL×2). The organic phase was washed with brine (100 mL×2), dried over anhydrous Na₂SO₄, filtered and concentrated under reduced pressure to give the title compound (10 g) as a yellow oil. ¹H NMR (400 MHz, CDCl₃-d) δ=4.16-4.04 (m, 2H), 2.39-2.27 (m, 1H), 2.09-1.93 (m, 4H), 1.84-1.72 (m, 2H), 1.67-1.43 (m, 2H), 1.28-1.19 (m, 3H).

Step 2—(4-Bromocyclohexyl)methanol. To a solution of LiAlH₄ (710 mg, 18.7 mmol) in THF (40 mL) was added ethyl 4-bromocyclohexanecarboxylate (4 g, 17 mmol). The mixture was stirred at 0° C. for 1 hour under N₂. On completion, the reaction mixture was quenched with H₂O (1 mL) at 0° C. then NaOH (15%, 2 mL) and the mixture was stirred at 20° C. for 15 min. The resulting solution was diluted with H₂O (30 mL) and extracted with EA (50 mL×2). The combined organic layers were washed with brine (50 mL×2), dried over Na₂SO₄, filtered under reduced pressure, the filtrate was concentrated under reduced pressure to give the title compound (3 g) as a yellow oil. ¹H NMR (400 MHz, CHLOROFORM-d) δ=3.48-3.42 (m, 2H), 3.38 (t, J=6.8 Hz, 1H), 2.09-1.99 (m, 2H), 1.81-1.71 (m, 4H), 1.61-1.57 (m, 1H), 1.53-1.46 (m, 2H).

Step 3—((4-Bromocyclohexyl)methoxy)(tert-butyl)diphenylsilane. To a solution of (4-bromocyclohexyl)methanol (4 g, 20.7 mmol) and TBDPSCl (8.54 g, 31.1 mmol) in DMF (50 mL) was added imidazole (1.41 g, 20.7 mmol) at 0° C. The mixture was stirred at 20° C. for 3 hrs. On completion, the reaction mixture was diluted with H₂O (50 mL) and extracted with EA (50 mL×2). The combined organic layers were washed with brine (50 mL×2), dried over Na₂SO₄, filtered and concentrated under reduced pressure to give a residue. The residue was purified by column chromatography (SiO₂, Petroleum ether/Ethyl acetate=10/1 to 5/1) to give the title compound (5 g, 56% yield) as a colorless oil.

Step 4—(R)-methyl 1-((2-aminobutyl)amino)thieno[3,2-f]quinoline-2-carboxylate. To an 40 mL vial equipped with a stir bar was added (4-bromocyclohexyl)methoxy-tert-butyl-diphenyl-silane (2.96 g, 6.87 mmol), 2-bromo-5-(4,4,5,5-tetramethyl-1,3,2-dioxaborolan-2-yl)pyridine (1.5 g, 5.28 mmol), Ir[dF(CF₃)ppy]₂(dtbpy)(PF₆) (59.3 mg, 52.8 umol), NiCl₂.dtbbpy (105 mg, 264 umol), TTMSS (1.31 g, 5.28 mmol), and Na₂CO₃ (1.12 g, 10.6 mmol) in DME (20 mL). The vial was sealed, stirred and irradiated with a 10 W blue LED lamp (3 cm away), with cooling water to keep the reaction temperature at 25° C. for 14 hrs. On completion, the reaction mixture was filtered and concentrated under reduced pressure to give a residue. The residue was purified by column chromatography (SiO₂, Petroleum ether/Ethyl acetate=10/1 to 1/1) to give the title compound as a yellow oil. LC-MS (ESI⁺) m/z 474.4 (M+H)⁺.

### (R)-3-(6-((1r,4r)-4-(((tert-butyldiphenylsilyl)oxy)methyl)cyclohexyl)pyridin-3-yl)-10-methyl-9,10,11,12-tetrahydro-8H-[1,4]diazepino[5′,6′:4,5]thieno[3,2-f]quinolin-8-one (Intermediate DH)

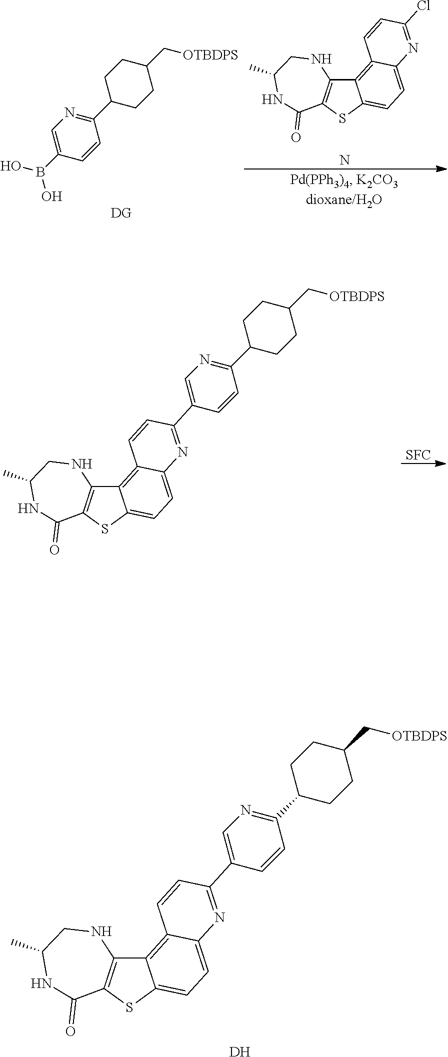

Step 1—(R)-3-(6-(4-(((tert-butyldiphenylsilyl)oxy)methyl)cyclohexyl)pyridin-3-yl)-10-methyl-9,10,11,12-tetrahydro-8H-[1,4]diazepino[5′,6′:4,5]thieno[3,2-f]quinolin-8-one. A mixture of (6-(4-(((tert-butyldiphenylsilyl)oxy)methyl)cyclohexyl)pyridin-3-yl)boronic acid (581 mg, 1.23 mmol, Intermediate DG), (R)-3-chloro-10-methyl-9,10,11,12-tetrahydro-8H-[1,4]diazepino[5′,6′:4,5]thieno[3,2-f]quinolin-8-one (300 mg, 944 umol, Intermediate N), K₂CO₃ (391 mg, 2.83 mmol), Pd(PPh₃)₄(109 mg, 94.4 umol) in dioxane (8 mL) and H₂O (1 mL) was degassed and purged with N₂ three times. Then the mixture was stirred at 80° C. for 2 hrs under N₂ atmosphere. On completion, the reaction mixture was diluted with H₂O (50 mL) and extracted with EA (50 mL×2). The combined organic layers were dried over Na₂SO₄, filtered and concentrated under reduced pressure to give a residue. The residue was purified by prep-HPLC (0.1% FA condition) to give the title compound (180 mg, 26% yield) as a yellow solid. LC-MS (ESI⁺) m/z 711.5 (M+H)⁺.

Step 2—(R)-3-(6-((1r,4r)-4-(((tert-butyldiphenylsilyl)oxy)methyl)cyclohexyl)pyridin-3-yl)-10-methyl-9,10,11,12-tetrahydro-8H-[1,4]diazepino[5′,6′:4,5]thieno[3,2-f]quinolin-8-one. (R)-3-(6-(4-(((tert-butyldiphenylsilyl)oxy)methyl)cyclohexyl)pyridin-3-yl)-10-methyl-9,10,11,12-tetrahydro-8H-[1,4]diazepino[5′,6′:4,5]thieno[3,2-f]quinolin-8-one was separated by SFC (column: DAICEL CHIRALPAK IG (250 mm×30 mm, 10 um); mobile phase: [0.1% NH₃H₂O MEOH]; B %: 60%-60%, 7; 95 min) to give the title compound (90 mg) as a yellow solid. ¹H NMR (400 MHz, DMSO-d₆) δ=9.40 (d, J=2.0 Hz, 1H), 9.29 (d, J=9.2 Hz, 1H), 8.61-8.53 (m, 1H), 8.31 (d, J=9.2 Hz, 1H), 8.17 (d, J=8.8 Hz, 1H), 8.12 (d, J=4.0 Hz, 1H), 8.04 (d, J=8.8 Hz, 1H), 7.69-7.64 (m, 4H), 7.51-7.45 (m, 7H), 7.19 (t, J=4.8 Hz, 1H), 3.68-3.61 (m, 1H), 3.57 (d, J=6.0 Hz, 2H), 3.51 (s, 2H), 2.81-2.66 (m, 1H), 1.96 (t, J=15.2 Hz, 4H), 1.70-1.58 (m, 3H), 1.25-1.20 (m, 5H), 1.05 (s, 9H).

### (1r,4r)-4-(5-((R)-10-methyl-8-oxo-9,10,11,12-tetrahydro-8H-[1,4]diazepino[5′,6′:4,5]thieno[3,2-f]quinolin-3-yl)pyridin-2-yl)cyclohexanecarbaldehyde (Intermediate DI)

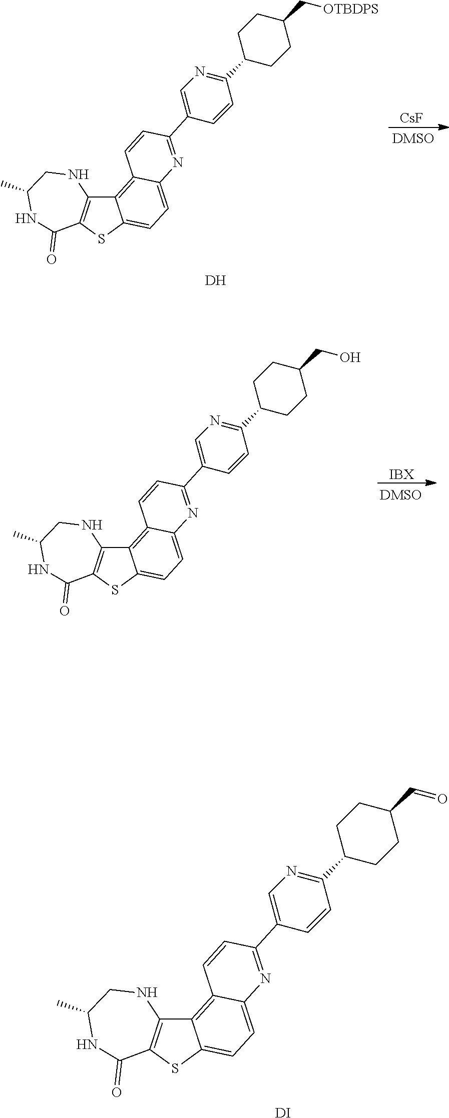

Step 1—(R)-3-(6-((1r,4r)-4-(hydroxymethyl)cyclohexyl)pyridin-3-yl)-10-methyl-9,10,11,12-tetrahydro-8H-[1,4]diazepino[5′,6′:4,5]thieno[3,2-f]quinolin-8-one. To a solution of (R)-3-(6-((1r,4r)-4-(((tert-butyldiphenylsilyl)oxy)methyl)cyclohexyl)pyridin-3-yl)-10-methyl-9,10,11,12-tetrahydro-8H-[1,4]diazepino[5′,6′:4,5]thieno[3,2-f]quinolin-8-one (80 mg, 113 umol, Intermediate DH) in DMSO (1 mL) was added CsF (171 mg, 1.13 mmol). The mixture was stirred at 40° C. for 12 hrs. On completion, the reaction mixture was filtered under reduced pressure. The filtrate was purified by prep-HPLC (0.1% NH₄HCO₃ condition) to give the title compound (40 mg, 65% yield) as a yellow solid. LC-MS (ESI⁺) m/z 473.2 (M+H)⁺.

Step 2—(1r,4r)-4-(5-((R)-10-methyl-8-oxo-9,10,11,12-tetrahydro-8H-[1,4]diazepino[5′,6′:4,5]thieno[3,2-f]quinolin-3-yl)pyridin-2-yl)cyclohexanecarbaldehyde. To a solution of (R)-3-(6-((1r,4r)-4-(hydroxymethyl)cyclohexyl)pyridin-3-yl)-10-methyl-9,10,11,12-tetrahydro-8H-[1,4]diazepino[5′,6′:4,5]thieno[3,2-f]quinolin-8-one (35 mg, 74.1 umol) in DMSO (0.5 mL) was added IBX (41.5 mg, 148 umol). The mixture was stirred at 20° C. for 12 hrs. On completion, the reaction mixture was quenched with aqueous NaHCO₃ (5 mL) at 20° C. and then diluted with H₂O (20 mL) and extracted with EA (20 mL×3). The combined organic layers were washed with brine (20 mL×2), dried over Na₂SO₄, filtered and concentrated under reduced pressure to give the title compound (35 mg) as a yellow solid. LC-MS (ESI⁺) m/z 471.3 (M+H)⁺.

### Tert-butyl 4-(3-ethyl-5-(4,4,5,5-tetramethyl-1,3,2-dioxaborolan-2-yl)phenyl)piperazine-1-carboxylate (Intermediate DJ)

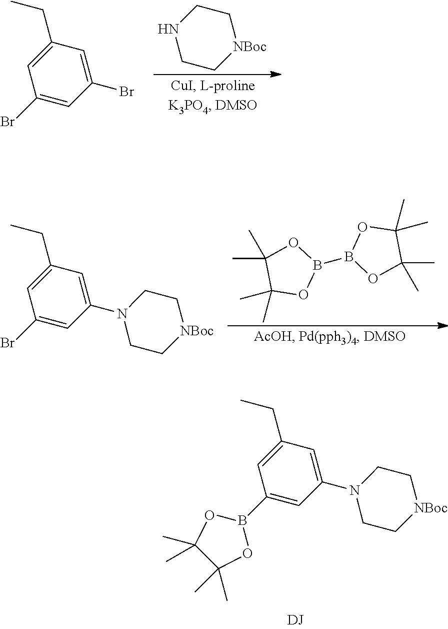

Step 1—Tert-butyl 4-(3-bromo-5-ethylphenyl)piperazine-1-carboxylate. To a solution of 1,3-dibromo-5-ethylbenzene (4.9 g, 18.6 mmol) and tert-butyl piperazine-1-carboxylate (2.30 g, 12.4 mmol) in DMSO (50 mL) was added CuI (3.54 g, 18.6 mmol), L-proline (285 mg, 2.48 mmol and K₂CO₃ (3.42 g, 24.8 mmol) at 25° C., then the mixture was stirred at 80° C. for 2 hrs. On completion, the reaction mixture was quenched with water (60 mL) and extracted with ethyl acetate (40×5 mL). The extracts were washed with brine (300 mL) and dried over anhydrous sodium sulfate, filtered and concentrated in vacuo to give the crude residue. The residue was purified by column chromatography (SiO₂, Petroleum ether/Ethyl acetate=15/1 to 10/1) to give the title compound (400 mg, 9% yield) as a colorless oil. LC-MS (ESI⁺) m/z 313.0. (M+H)⁺.

Step 2—Tert-butyl 4-(3-ethyl-5-(4,4,5,5-tetramethyl-1,3,2-dioxaborolan-2-yl)phenyl)piperazine-1-carboxylate. To a solution of tert-butyl 4-(3-bromo-5-ethyl-phenyl)piperazine-1-carboxylate (400 mg, 1.08 mmol) and 4,4,4′,4′,5,5,5′,5′-octamethyl-2,2′-bi(1,3,2-dioxaborolane) (825 mg, 3.25 mmol) in DMSO (8 mL) was added KOAc (319 mg, 3.25 mmol) and Pd(PPh₃)₄(125 mg, 108 umol) at 25° C., then the mixture was stirred at 80° C. for 2 hrs. On completion, the reaction mixture was quenched with water (30 mL) and extracted with ethyl acetate (20×4 mL). The extracts were then washed with brine (100 mL) and dried over anhydrous sodium sulfate, filtered and concentrated in vacuo to give the crude residue. The residue was purified by column chromatography (SiO₂, Petroleum ether/Ethyl acetate=15/1 to 10/1) to give the title compound (400 mg, 60% yield) as yellow solid. LC-MS (ESI⁺) m/z 416.9. (M+H)⁺.

### (R)-3-(3-ethyl-5-(piperazin-1-yl)phenyl)-10-methyl-9,10,11,12-tetrahydro-8H-[1,4]diazepino[5′,6′:4,5]thieno[3,2-f]quinolin-8-one (Intermediate DK)

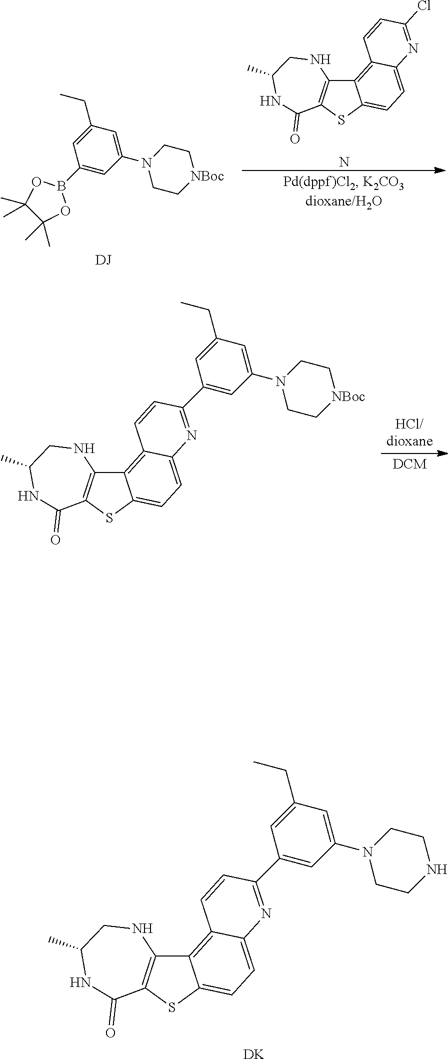

Step 1—(R)-tert-butyl 4-(3-ethyl-5-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. To a solution of tert-butyl 4-(3-ethyl-5-(4,4,5,5-tetramethyl-1,3,2-dioxaborolan-2-yl)phenyl)piperazine-1-carboxylate (178 mg, 560 umol, Intermediate DJ) and (R)-3-chloro-10-methyl-9,10,11,12-tetrahydro-8H-[1,4]diazepino[5′,6′:4,5]thieno[3,2-f]quinolin-8-one (350 mg, 841 umol, Intermediate N) in dioxane (4 mL) and H₂O (0.8 mL) was added Pd-PEPPSI-IHeptCl (54.5 mg, 56.0 umol) and Cs₂CO₃ (548 mg, 1.68 mmol) at 25° C. Then the mixture was stirred at 80° C. for 2 hrs. On completion, the reaction mixture was filtered and concentrated in vacuo to give the crude residue. The residue was purified by column chromatography (SiO₂, Dichloromethane/THF=1/1) to give the title compound (250 mg, 56% yield) as yellow solid. LC-MS (ESI⁺) m/z 571.2. (M+H)⁺.

Step 2—(R)-3-(3-ethyl-5-(piperazin-1-yl)phenyl)-10-methyl-9,10,11,12-tetrahydro-8H-[1,4]diazepino[5′,6′:4,5]thieno[3,2-f]quinolin-8-one. To a solution of (R)-tert-butyl 4-(3-ethyl-5-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (100 mg, 175 umol) in DCM (1 mL) was added HCl/dioxane (4 M, 0.3 mL) at 25° C., then the mixture was stirred at 25° C. for 1 hr. On completion, the reaction mixture was filtered and concentrated in vacuo to give the title compound (100 mg, HCl) as yellow solid. LC-MS (ESI⁺) m/z 472.1. (M+H)⁺.

### (15R)-15-methyl-5-(2-methyl-6-piperazin-1-yl-3-pyridyl)-11-thia-6,14,17-triazatetracyclo[8.8.0.0^{2,7}.0^{12,18}]octadeca-1,3,5,7,9,12(18)-hexaen-13-one (Intermediate DL)

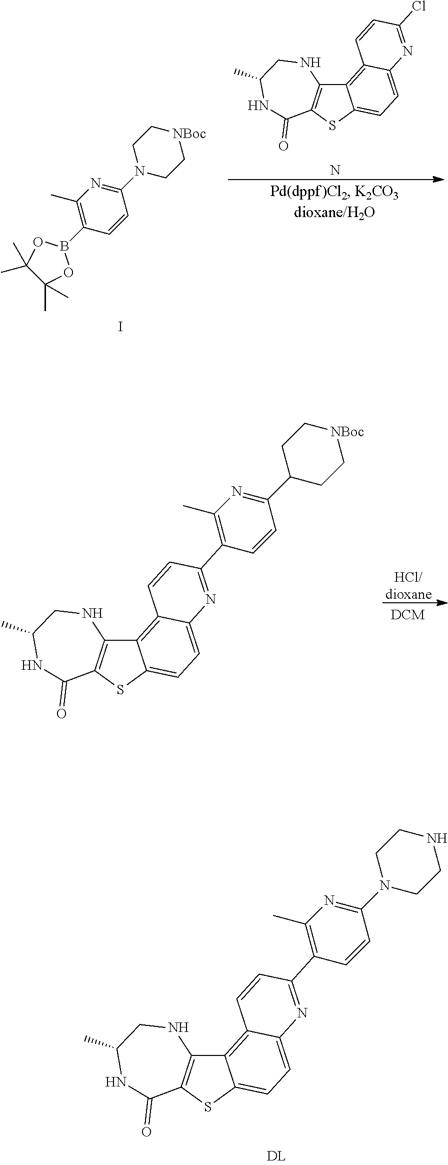

Step 1—(R)-tert-butyl 4-(6-methyl-5-(10-methyl-8-oxo-9,10,11,12-tetrahydro-8H-[1,4]diazepino[5′,6′:4,5]thieno[3,2-f]quinolin-3-yl)pyridin-2-yl)piperazine-1-carboxylate. A mixture of (15R)-5-chloro-15-methyl-11-thia-6,14,17-triazatetracyclo[8.8.0.0^{2,7}.0^{12,18}]octadeca-1,3,5,7,9,12(18)-hexaen-13-one (200 mg, 629 umol, Intermediate N), tert-butyl 4-[6-methyl-5-(4,4,5,5-tetramethyl-1,3,2-dioxaborolan-2-yl)-2-pyridyl]piperazine-1-carboxylate (381 mg, 944 umol, Intermediate I), Pd(dppf)Cl₂ (46.1 mg, 62.9 umol), and K₂CO₃ (261 mg, 1.89 mmol) in dioxane (15 mL) and H₂O (2 mL) was degassed and purged with N₂ three times. Then the mixture was stirred at 60° C. for 2 hrs under N₂ atmosphere. On completion, the reaction was filtered and the filtrate was concentrated under reduced pressure to give a residue. The residue was triturated with Petroleum ether:Ethyl acetate=10:1 (60 mL) at 25° C. for 20 min to give the title compound (500 mg) as a brown solid. LC-MS (ESI⁺) m/z 559.1 (M+H)⁺

Step 2—(R)-10-methyl-3-(2-methyl-6-(piperazin-1-yl)pyridin-3-yl)-9,10,11,12-tetrahydro-8H-[1,4]diazepino[5′,6′:4,5]thieno[3,2-f]quinolin-8-one. To a solution of tert-butyl 4-[6-methyl-5-[(15R)-15-methyl-13-oxo-11-thia-6,14,17-triazatetracyclo[8.8.0.0^{2,7}.0^{12,18}]octadeca-1,3,5,7,9,12(18)-hexaen-5-yl]-2-pyridyl]piperazine-1-carboxylate (200 mg, 358 umol) in DCM (5 mL) was added HCl/dioxane (1 M, 1 mL). The mixture was stirred at 25° C. for 1 hr. On completion, the reaction mixture was concentrated under vacuum to give the title compound (200 mg, HCl salt) as a red solid. LC-MS (ESI⁺) m/z 459.1 (M+H)⁺.

### (Tert-butyl 4-(3-methyl-4-(4,4,5,5-tetramethyl-1,3,2-dioxaborolan-2-yl)phenyl)piperazine-1-carboxylate (Intermediate DM)

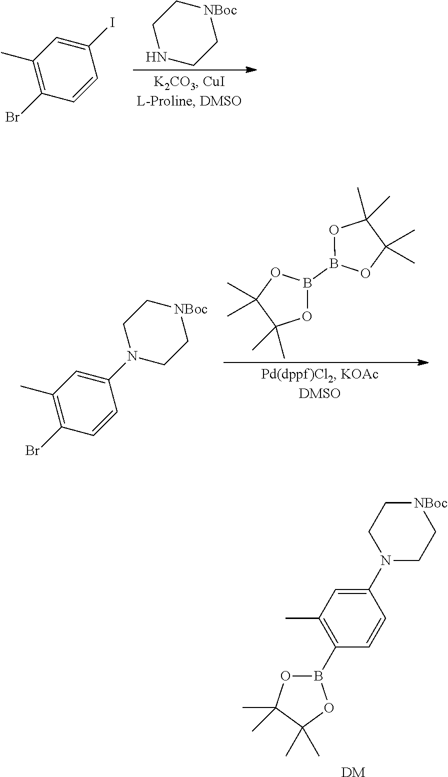

Step 1—Tert-butyl 4-(4-bromo-3-methylphenyl)piperazine-1-carboxylate. A mixture of 1-bromo-4-iodo-2-methylbenzene (80 g, 26.9 mmol, CAS #589-87-7), tert-butyl piperazine-1-carboxylate (5 g, 26.9 mmol, CAS #143238-38-4), K₂CO₃ (7.45 g, 53.9 mmol), CuI (1.03 g, 5.39 mmol) and L-proline (1.24 g, 10.8 mmol) in DMSO (100 mL) was degassed and purged with N₂ three times. Then the mixture was stirred at 100° C. for 2 hrs under N₂ atmosphere. On completion, the reaction mixture was diluted with H₂O (200 mL) and extracted with EA (200 mL×3). The combined organic layers were washed with aqueous NaCl (200 mL×3), dried over Na₂SO₄, filtered and concentrated under reduced pressure to give a residue. The residue was purified by column chromatography (SiO₂, Petroleum ether/Ethyl acetate=10/0 to 10/1) to give the title compound (4.2 g, 40% yield) as a yellow solid. LC-MS (ESI⁺) m/z 355.0 (M+H)⁺.

Step 2—(Tert-butyl 4-(3-methyl-4-(4,4,5,5-tetramethyl-1,3,2-dioxaborolan-2-yl)phenyl)piperazine-1-carboxylate. A mixture of tert-butyl 4-(4-bromo-3-methylphenyl)piperazine-1-carboxylate (2 g, 5.63 mmol), 4,4,5,5-tetramethyl-2-(4,4,5,5-tetramethyl-1,3,2-dioxaborolan-2-yl)-1,3,2-dioxaborolane (2.86 g, 11.3 mmol), KOAc (1.66 g, 16.9 mmol), and Pd(dppf)Cl₂ (412 mg, 563 umol) in DMSO (40 mL) was degassed and purged with N₂ three times. Then the mixture was stirred at 80° C. for 12 hrs under N₂ atmosphere. On completion, the reaction mixture was diluted with H₂O (100 mL) and extracted with EA (100 mL×2). The combined organic layers were washed with aqueous NaCl (100 mL×2), dried over Na₂SO₄, filtered and concentrated under reduced pressure to give a residue. The residue was purified by column chromatography (SiO₂, Petroleum ether/Ethyl acetate=10/0 to 10/1) to give the title compound (600 mg, 23% yield) as a yellow solid. LC-MS (ESI⁺) m/z 403.3 (M+H)⁺

### (R)-10-methyl-3-(2-methyl-4-(piperazin-1-yl)phenyl)-9,10,11,12-tetrahydro-8H-[1,4]diazepino[5′,6′:4,5]thieno[3,2-f]quinolin-8-one (Intermediate DN)

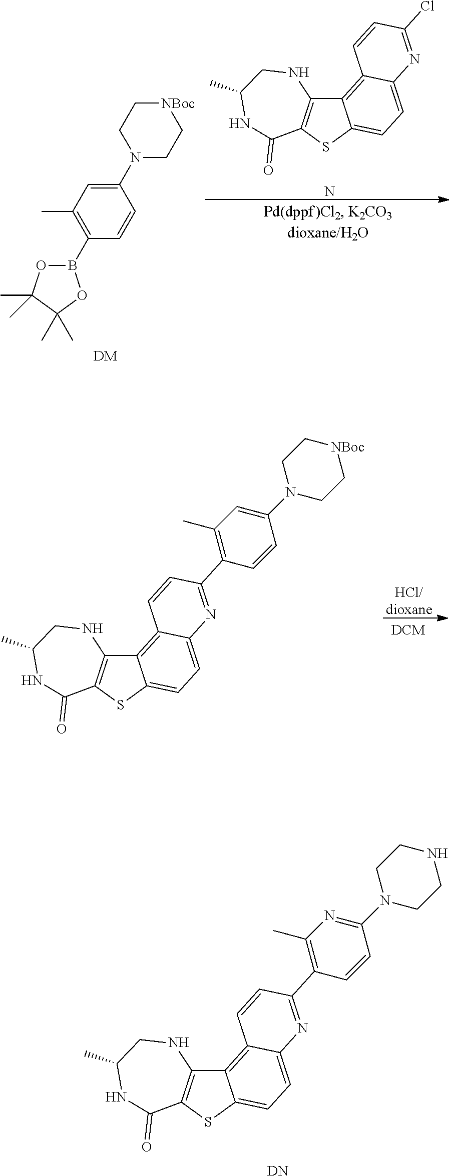

Step 1—(R)-tert-butyl 4-(3-methyl-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. A mixture of tert-butyl 4-(3-methyl-4-(4,4,5,5-tetramethyl-1,3,2-dioxaborolan-2-yl)phenyl)piperazine-1-carboxylate (600 mg, 1.49 mmol, Intermediate DM), (R)-3-chloro-10-methyl-9,10,11,12-tetrahydro-8H-[1,4]diazepino[5′,6′:4,5]thieno[3,2-f]quinolin-8-one (400 mg, 1.26 mmol, Intermediate N), K₂CO₃ (522 mg, 3.78 mmol), and Pd(dppf)Cl₂ (184 mg, 252 umol) in dioxane (10 mL) and H₂O (2 mL) was degassed and purged with N₂ three times. Then the mixture was stirred at 80° C. for 2 hrs under N₂ atmosphere. On completion, the reaction mixture was concentrated under reduced pressure to give a residue. The residue was triturated with EA at 20° C. for 10 min to give the title compound (800 mg, 68% yield) as an orange solid. LC-MS (ESI⁺) m/z 558.2 (M+H)⁺.

Step 2—(R)-10-methyl-3-(2-methyl-4-(piperazin-1-yl)phenyl)-9,10,11,12-tetrahydro-8H-[1,4]diazepino[5′,6′:4,5]thieno[3,2-f]quinolin-8-one. (R)-tert-butyl 4-(3-methyl-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (200 mg, 359 umol) in DCM (3 mL) was added HCl/dioxane (0.5 mL, 4 M). The mixture was stirred at 25° C. for 1 min. On completion, the reaction mixture was concentrated under reduced pressure to give the title compound (200 mg) as a red solid. LC-MS (ESI⁺) m/z 458.2 (M+H)⁺.

### 3-[1-(2,6-dioxo-3-piperidyl)-3-methyl-2-oxo-benzimidazol-5-yl]propanal (Intermediate DO)

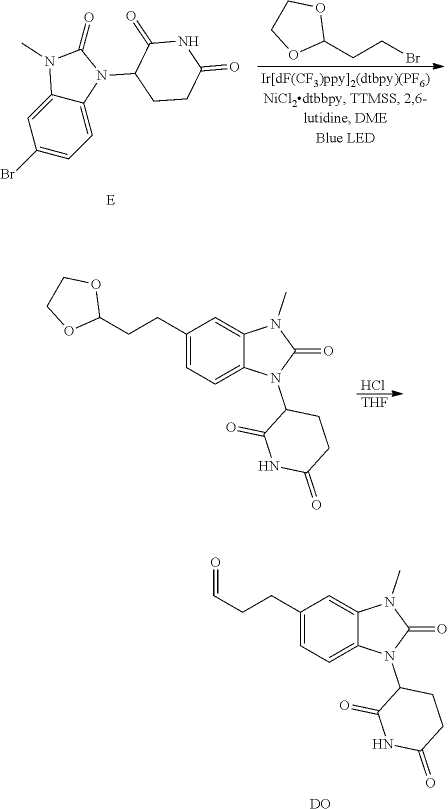

Step 1—3-(5-(2-(1,3-Dioxolan-2-yl)ethyl)-3-methyl-2-oxo-2,3-dihydro-1H-benzo[d]imidazol-1-yl)piperidine-2,6-dione. To a vial equipped with a stir bar was added 3-(5-bromo-3-methyl-2-oxo-2,3-dihydro-1H-benzo[d]imidazol-1-yl)piperidine-2,6-dione (3 g, 8.87 mmol, Intermediate E), 2-(2-bromoethyl)-1,3-dioxolane (2.09 g, 11.5 mmol, CAS #18742-02-4), Ir[dF(CF₃)ppy]₂(dtbpy)(PF₆) (100 mg, 88.7 umol), NiCl₂.dtbbpy (53 mg, 113 umol), TTMSS (2.21 g, 8.87 mmol), and 2,6-lutitine (3.27 g, 17.7 mmol) in DME (60 L). The vial was sealed and placed under nitrogen was added. The reaction was stirred and irradiated with a 10 W blue LED lamp (3 cm away), with cooling water to keep the reaction temperature at 25° C. for 14 hrs. On completion the reaction mixture was filtered and concentrated under reduced pressure to give a residue. The residue was purified by column chromatography (SiO₂, Petroleum ether/Ethyl acetate=10/1 to 0/1) to give the title compound (1.5 g, 47% yield) as a yellow solid. LC-MS (ESI⁺) m/z 360.2 (M+H)⁺.

Step 2—3-(1-(2,6-dioxopiperidin-3-yl)-3-methyl-2-oxo-2,3-dihydro-1H-benzo[d]imidazol-5-yl)propanal. To a solution of tert-butyl 3-(5-(2-(1,3-dioxolan-2-yl)ethyl)-3-methyl-2-oxo-2,3-dihydro-1H-benzo[d]imidazol-1-yl)piperidine-2,6-dione (120 mg, 334 umol) in THF (5 mL) was added HCl (1 M, 1 mL). The mixture was stirred at 50° C. for 1 hr. On completion, the reaction mixture was diluted with H₂O (20 mL) and extracted with EA (20 mL×3). The combined organic layers were dried over Na₂SO₄, filtered and concentrated under reduced pressure to give the title compound (100 mg) as a yellow solid. LC-MS (ESI⁺) m/z 315.9 (M+H)⁺.

### (R)-3-(2-fluoro-4-(piperazin-1-yl)phenyl)-10-methyl-9,10,11,12-tetrahydro-8H-[1,4]diazepino[5′,6′:4,5]thieno[3,2-f]quinolin-8-one (Intermediate DP)

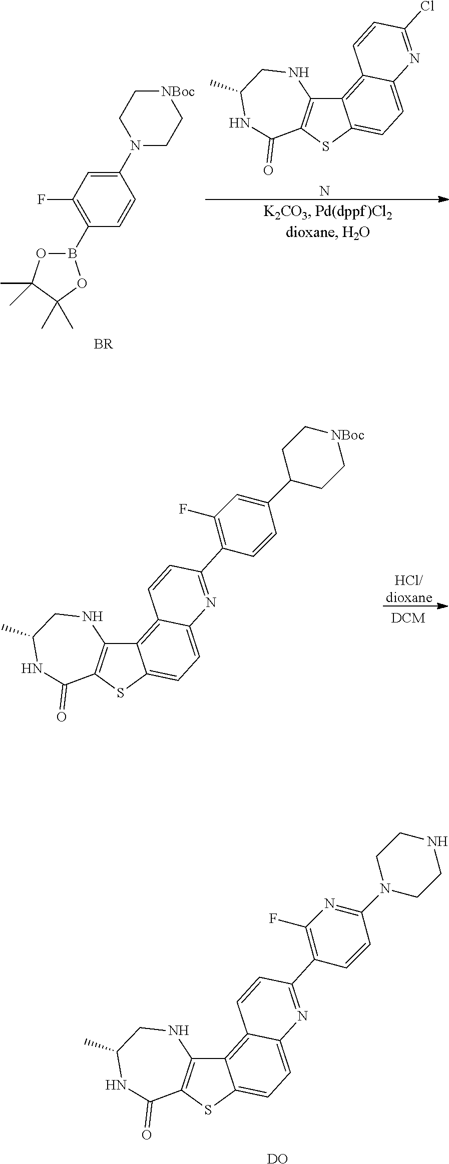

Step 1—(R)-tert-butyl 4-(3-fluoro-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. To a solution of tert-butyl 4-[3-fluoro-4-(4,4,5,5-tetramethyl-1,3,2-dioxaborolan-2-yl)phenyl]piperazine-1-carboxylate (843.81 mg, 2.08 mmol, Intermediate BR) and (15R)-5-chloro-15-methyl-11-thia-6,14,17-triazatetracyclo[8.8.0.0^{2,7}.0^{12,18}]octadeca-1,3,5,7,9,12(18)-hexaen-13-one (550 mg, 1.73 mmol, Intermediate N) in dioxane (16 mL) and H₂O (4 mL) was added K₂CO₃ (717 mg, 5.19 mmol) and Pd(dppf)Cl₂ (126 mg, 173 umol). The mixture was degassed and purged with N₂ three times, then stirred at 80° C. for 2 hrs under N₂ atmosphere. The reaction mixture was diluted with H₂O (20 mL) and extracted with EA (20 mL×3). The combined organic layers were added PE, then stirred 5 minutes, filtered and concentrated under reduced pressure to give the title compound (880 mg) as a brown solid. LC-MS (ESI⁺) m z 562.2 (M+1)⁺.

Step 2—(R)-3-(2-fluoro-4-(piperazin-1-yl)phenyl)-10-methyl-9,10,11,12-tetrahydro-8H-[1,4]diazepino[5′,6′:4,5]thieno[3,2-f]quinolin-8-one. To a solution of tert-butyl 4-[3-fluoro-4-[(15R)-15-methyl-13-oxo-11-thia-6,14,17-triazatetracyclo[8.8.0.0^{2,7}.0^{12,18}]octadeca-1,3,5,7,9,12(18)-hexaen-5-yl]phenyl]piperazine-1-carboxylate (240 mg, 427 umol) in DCM (4 mL) was added HCl/dioxane (8 M, 0.5 mL). The mixture was stirred at 25° C. for 0.5 hr. On completion, the reaction mixture was concentrated under reduced pressure to the title compound (250 mg, HCl) as a red solid. LC-MS (ESI⁺) m z 562.2 (M+1)⁺.

### Tert-butyl 4-(3-methoxy-5-(4,4,5,5-tetramethyl-1,3,2-dioxaborolan-2-yl)phenyl)piperazine-1-carboxylate (Intermediate DQ)

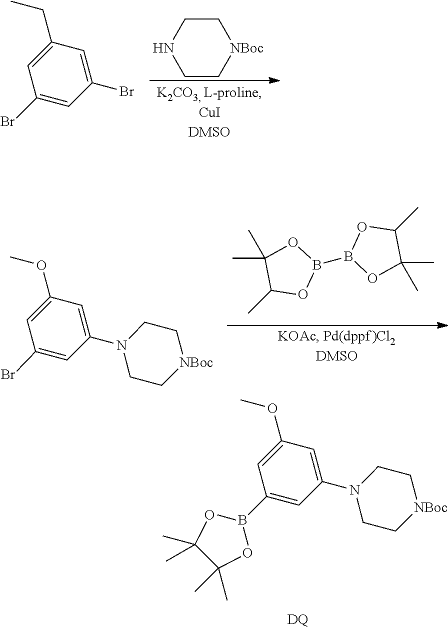

Step 1—Tert-butyl 4-(3-bromo-5-methoxyphenyl)piperazine-1-carboxylate. A mixture of 1,3-dibromo-5-methoxy-benzene (5 g, 18.8 mmol, CAS #74137-36-3), tert-butyl piperazine-1-carboxylate (3.50 g, 18.8 mmol, CAS #143238-38-4), K₂CO₃ (5.20 g, 37.60 mmol), L-PROLINE (866 mg, 7.52 mmol) and CuI (716 mg, 3.76 mmol) in DMSO (100 mL) was degassed and purged with N₂ three times. Then the mixture was stirred at 100° C. for 3 hr under N₂ atmosphere. On completion, the reaction mixture was diluted with H₂O (100 mL) and extracted with EA (100 mL×3). The combined organic layers were washed with aqueous NaCl (100 mL×3), dried over Na₂SO₄, filtered and concentrated under reduced pressure to give a residue. The residue was purified by column chromatography (SiO₂, Petroleum ether/Ethyl acetate=10/1 to 5/1) to give the title compound (1.3 g, 18% yield) as a white solid. LC-MS (ESI⁺) m/z 373.0 (M+H)⁺.

Step 2—Tert-butyl 4-(3-methoxy-5-(4,4,5,5-tetramethyl-1,3,2-dioxaborolan-2-yl)phenyl)piperazine-1-carboxylate. A mixture of tert-butyl 4-(3-bromo-5-methoxyphenyl)piperazine-1-carboxylate (500 mg, 1.35 mmol), 4,4,4′,4′,5,5′-hexamethyl-2,2′-bi(1,3,2-dioxaborolane) (686 mg, 2.70 mmol), KOAc (397 mg, 4.05 mmol), Pd(dppf)Cl₂ (98.8 mg, 135 umol) in DMSO (10 mL) was degassed and purged with N₂ three times. Then the mixture was stirred at 80° C. for 2 hr under N₂ atmosphere. On completion, the reaction mixture was diluted with H₂O (50 mL) and extracted with EA (50 mL×3). The combined organic layers were washed with aqueous NaCl (50 mL×3), dried over Na₂SO₄, filtered and concentrated under reduced pressure to give a residue. The residue was purified by column chromatography (SiO₂, Petroleum ether/Ethyl acetate=10/1 to 5/1) to give the title compound (350 mg, 59% yield) as a yellow solid. LC-MS (ESI⁺) m/z 419.1 (M+H)⁺.

### ((R)-3-(3-methoxy-5-(piperazin-1-yl)phenyl)-10-methyl-9,10,11,12-tetrahydro-8H-[1,4]diazepino[5′,6′:4,5]thieno[3,2-f]quinolin-8-one (Intermediate DR)

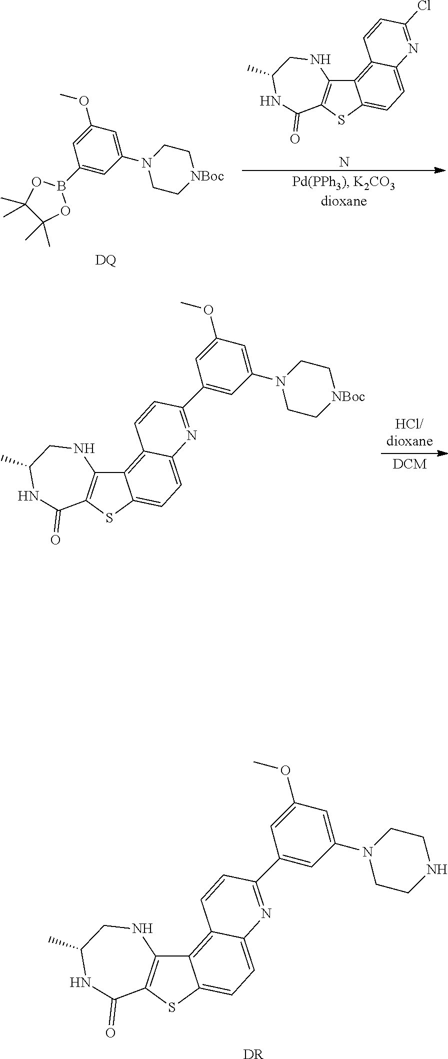

Step 1—(R)-tert-butyl 4-(3-methoxy-5-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. A mixture of tert-butyl 4-(3-methoxy-5-(4,4,5,5-tetramethyl-1,3,2-dioxaborolan-2-yl)phenyl)piperazine-1-carboxylate (200 mg, 478 umol, Intermediate DQ), (R)-3-chloro-10-methyl-9,10,11,12-tetrahydro-8H-[1,4]diazepino[5′,6′:4,5]thieno[3,2-f]quinolin-8-one (129 mg, 406 umol, Intermediate N), K₂CO₃ (198 mg, 1.43 mmol) and Pd(PPh₃)₄(55.2 mg, 47.8 umol) in dioxane (3 mL) and H₂O (0.5 mL) was degassed and purged with N₂ three times. Then the mixture was stirred at 80° C. for 2 hr under N₂ atmosphere. On completion, the reaction mixture was diluted with H₂O (20 mL) and extracted with EA (20 mL×3). The combined organic layers were washed with aqueous NaCl (20 mL×3), dried over Na₂SO₄, filtered and concentrated under reduced pressure to give a residue. The residue was triturated with EA at 20° C. for 10 min to give the title compound (210 mg, 67% yield) as an orange solid. LC-MS (ESI⁺) m/z 574.4 (M+H)⁺.

Step 2—((R)-3-(3-methoxy-5-(piperazin-1-yl)phenyl)-10-methyl-9,10,11,12-tetrahydro-8H-[1,4]diazepino[5′,6′:4,5]thieno[3,2-f]quinolin-8-one. To a solution of (R)-tert-butyl 4-(3-methoxy-5-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (210 mg, 366 umol) in DCM (2 mL) was added HCl/dioxane (4 M, 915 uL). The mixture was stirred at 20° C. for 10 min. On completion, the reaction mixture was concentrated under reduced pressure to give a residue. The residue was purified by prep-HPLC (1% FA condition) to give the title compound (12.9 mg) as a red solid. ¹H NMR (400 MHz, DMSO-d₆) δ=9.21 (d, J=9.2 Hz, 1H), 8.26 (s, 1H), 8.23 (d, J=9.2 Hz, 1H), 8.15-8.11 (m, 1H), 8.09 (d, J=4.4 Hz, 1H), 8.02 (d, J=9.2 Hz, 1H), 7.45 (s, 1H), 7.29 (s, 1H), 7.16 (br t, J=5.2 Hz, 1H), 6.61 (t, J=2.0 Hz, 1H), 3.86 (s, 3H), 3.63-3.60 (m, 1H), 3.48 (br s, 2H), 3.25-3.22 (m, 4H), 2.98-2.93 (m, 4H), 1.21 (d, J=6.8 Hz, 3H). LC-MS (ESI⁺) m/z 473.3 (M+H)⁺.

### (3-Bromocyclobutoxy)(tert-butyl)dimethylsilane (Intermediate DS)

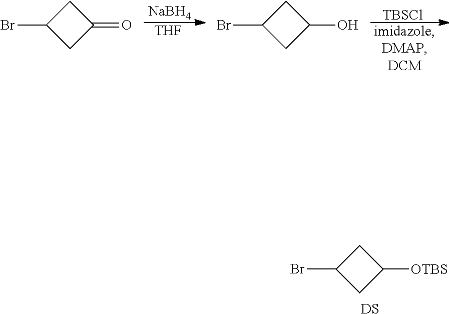

Step 1—3-Bromocyclobutanol. To a solution of 3-bromocyclobutanone (2 g, 13.4 mmol, CAS #2371-24-2) in THF (50 mL) was added NaBH₄ (559 mg, 14.8 mmol). The mixture was stirred at 0° C. for 2 hrs. On completion, the reaction mixture was quenched with H₂O (10 mL) at 0° C., and was extracted with ethyl acetate (3×50 mL). The combined organic layer was dried over Na₂SO₄, filtered and the filtrate was concentrated under reduced pressure to give the title compound (2 g) as a colorless oil.

Step 2—(3-Bromocyclobutoxy)(tert-butyl)dimethylsilane. To a solution of 3-bromocyclobutanol (2 g, 13.2 mmol) in DCM (30 mL) was added imidazole (1.80 g, 26.5 mmol), DMAP (162 mg, 1.32 mmol) and TBSCl (2.99 g, 19.9 mmol, 2.43 mL), then the reaction was stirred at 25° C. for 12 hrs. On completion, the reaction mixture was diluted with sat. NH₄Cl (10 mL) and extracted with DCM (3×10 mL). The combined organic layers were washed with brine, dried over Na₂SO₄, filtered and the filtrate was concentrated under reduced pressure to give a residue. The residue was purified by column chromatography (SiO₂, Petroleum ether/Ethyl acetate=1/0 to 10/1) to give the title compound (1.2 g, 34% yield) as a colorless oil. ¹H NMR (400 MHz, DMSO-d₆) δ=4.11-4.05 (m, 2H), 3.00-2.95 (m, 2H), 2.28-2.25 (m, 2H), 0.87 (s, 1H), 0.01 (s, 6H).

### 3-(3-methyl-2-oxo-4-(3-oxocyclobutyl)-2,3-dihydro-1H-benzo[d]imidazol-1-yl)piperidine-2,6-dione (Intermediate DT)

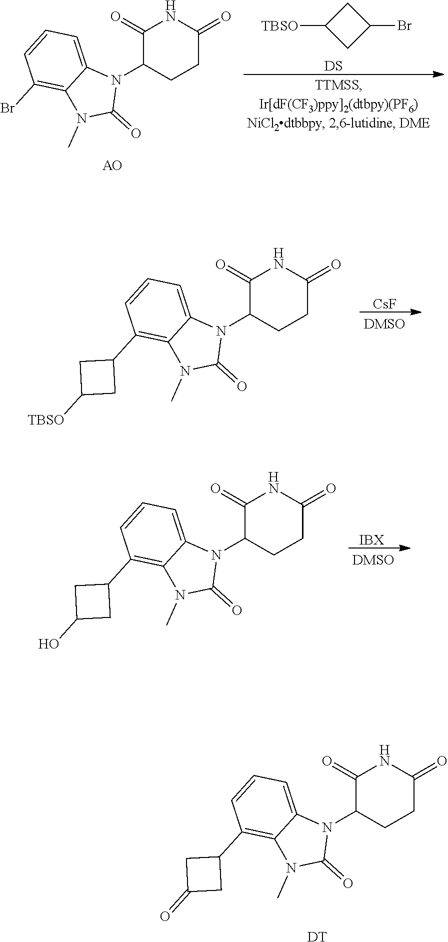

Step 1—3-(4-(3-((Tert-butyldimethylsilyl)oxy)cyclobutyl)-3-methyl-2-oxo-2,3-dihydro-1H-benzo[d]imidazol-1-yl)piperidine-2,6-dione. To a vial equipped with a stir bar was added 3-(4-bromo-3-methyl-2-oxo-2,3-dihydro-1H-benzo[d]imidazol-1-yl)piperidine-2,6-dione (2 g, 5.91 mmol, Intermediate AO), (3-bromocyclobutoxy)(tert-butyl)dimethylsilane (1.57 g, 5.91 mmol, Intermediate DS), TTMSS (1.47 g, 5.91 mmol), Ir[dF(CF₃)ppy]₂(dtbpy)(PF₆) (132 mg, 118 umol), NiCl₂.dtbbpy (70.6 mg, 177 umol), and 2,6-lutidine (1.27 g, 11.8 mmol) in DME (20 mL). The vial was sealed and placed under nitrogen was added. The reaction was stirred and irradiated with a 10 W blue LED lamp (3 cm away), with cooling water to keep the reaction temperature at 25° C. for 14 hrs. On completion, the reaction mixture was concentrated under reduced pressure to remove solvent. The residue was purified by column chromatography (SiO₂, Petroleum ether/Ethyl acetate=1/0 to 0/1) to give the title compound (2.1 g, 80% yield) as a yellow solid. LC-MS (ESI+) m/z 444.4 (M+H)⁺.

Step 2—3-(4-(3-Hydroxycyclobutyl)-3-methyl-2-oxo-2,3-dihydro-1H-benzo[d]imidazol-1-yl)piperidine-2,6-dione. To a solution of 3-(4-(3-((tert-butyldimethylsilyl)oxy)cyclobutyl)-3-methyl-2-oxo-2,3-dihydro-1H-benzo[d]imidazol-1-yl)piperidine-2,6-dione (2 g, 4.51 mmol) in DMSO (10 mL) was added CsF (6.85 g, 45.1 mmol). The mixture was stirred at 50° C. for 1 hr. On completion, the reaction mixture was quenched by addition H₂O (3 mL) at 0° C. The mixture was purified by reversed-phase HPLC (0.1% FA condition) to give the title compound (1 g, 66% yield) as a white solid. LC-MS (ESI+) m/z 330.1 (M+H)⁺.

Step 3—3-(3-Methyl-2-oxo-4-(3-oxocyclobutyl)-2,3-dihydro-1H-benzo[d]imidazol-1-yl)piperidine-2,6-dione. To a solution of 3-(4-(3-hydroxycyclobutyl)-3-methyl-2-oxo-2,3-dihydro-1H-benzo[d]imidazol-1-yl)piperidine-2,6-dione (400 mg, 1.21 mmol) in DMSO (6 mL) was added IBX (680 mg, 2.43 mmol). The mixture was stirred at 25° C. for 2 hrs. On completion, the reaction mixture was diluted with H₂O (30 mL) and extracted with EA (30 mL×3). The combined organic layers were washed with H₂O (30 mL×2), dried over Na₂SO₄, filtered and concentrated under reduced pressure to give a residue. The residue was purified by reversed-phase HPLC (0.1% FA condition) to give the title compound (340 mg, 84% yield) as a white solid. LC-MS (ESI⁺) m/z 328.2 (M+H)⁺.

### (R)-1-(4-(10-methyl-8-oxo-9,10,11,12-tetrahydro-8H-[1,4]diazepino[5′,6′:4,5]thieno[3,2-f]quinolin-3-yl)phenyl)piperidine-4-carbaldehyde (Intermediate DU)

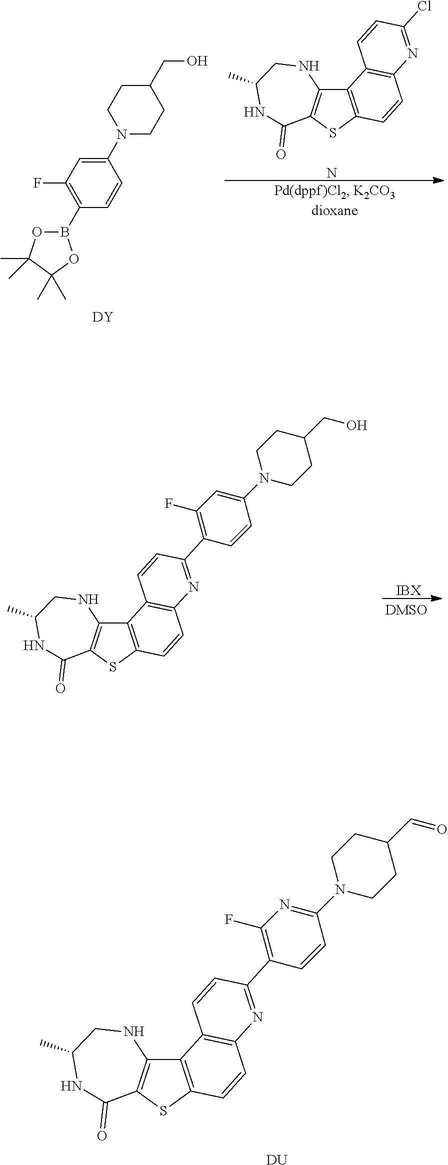

Step 1—(R)-3-(4-(4-(hydroxymethyl)piperidin-1-yl)phenyl)-10-methyl-9,10,11,12-tetrahydro-8H-[1,4]diazepino[5′,6′:4,5]thieno[3,2-f]quinolin-8-one. A mixture of (1-(4-(4,4,5,5-tetramethyl-1,3,2-dioxaborolan-2-yl)phenyl)piperidin-4-yl)methanol (200 mg, 630 umol, Intermediate DY), (R)-3-chloro-10-methyl-9,10,11,12-tetrahydro-8H-[1,4]diazepino[5′,6′:4,5]thieno[3,2-f]quinolin-8-one (200 mg, 630.umol, Intermediate N), K₂CO₃ (261 mg, 1.89 mmol), and Pd(dppf)Cl₂ (92.3 mg, 126 umol) in dioxane (5 mL) and H₂O (1 mL) was degassed and purged with N₂ three times. Then the mixture was stirred at 80° C. for 12 hrs under N₂ atmosphere. On completion, the reaction mixture was concentrated under reduced pressure to give a residue. The residue was purified by column chromatography (SiO₂, Petroleum ether/Ethyl acetate=10/1 to DCM/MeOH=10/1) to give the title compound (200 mg, 46% yield) as an orange solid. LC-MS (ESI⁺) m/z 473.3 (M+H)⁺.

Step 2—(R)-1-(4-(10-methyl-8-oxo-9,10,11,12-tetrahydro-8H-[1,4]diazepino[5′,6′:4,5]thieno[3,2-f]quinolin-3-yl)phenyl)piperidine-4-carbaldehyde. To a solution of (R)-3-(4-(4-(hydroxymethyl)piperidin-1-yl)phenyl)-10-methyl-9,10,11,12-tetrahydro-8H-[1,4]diazepino[5′,6′:4,5]thieno[3,2-f]quinolin-8-one (200 mg, 423 umol) in DMSO (5 mL) was added IBX (237 mg, 846 umol). The mixture was stirred at 20° C. for 2 hrs. On completion, the reaction mixture was diluted with H₂O (20 mL) and extracted with EA (20 mL×3). The combined organic layers were washed with brine (20 mL×4), dried over Na₂SO₄, filtered and the filtrate was concentrated under reduced pressure give the title compound (200 mg) as a red solid. LC-MS (ESI⁺) m/z 471.3 (M+H)⁺.

### (R)-1-(4-(4-(6-methyl-4-oxo-4,5,6,7-tetrahydro-1H-pyrrolo[3,2-c]pyridin-2-yl)pyridin-2-yl)phenyl)piperidine-4-carbaldehyde (Intermediate DV)

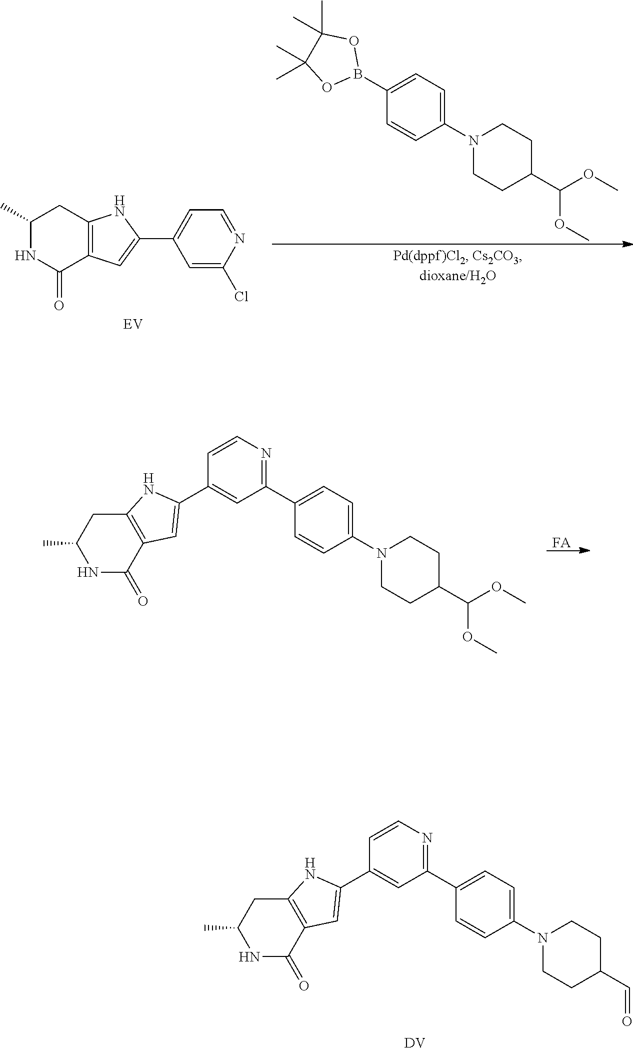

Step 1 (R)-2-(2-(4-(4-(dimethoxymethyl)piperidin-1-yl)phenyl)pyridin-4-yl)-6-methyl-6,7-dihydro-1H-pyrrolo[3,2-c]pyridin-4(5H)-one. To a solution of (R)-2-(2-chloropyridin-4-yl)-6-methyl-6,7-dihydro-1H-pyrrolo[3,2-c]pyridin-4(5H)-one (275 mg, 1.05 mmol, Intermediate EV) and 4-(dimethoxymethyl)-1-(4-(4,4,5,5-tetramethyl-1,3,2-dioxaborolan-2-yl)phenyl)piperidine (500 mg, 1.58 mmol, Intermediate CI) in dioxane (7 mL) and H₂O (1 mL) was added Cs₂CO₃ (1.03 g, 3.15 mmol) and Pd-PEPPSI-IHeptCl (102 mg, 105 umol) at 25° C., then the mixture was stirred at 80° C. for 2 hrs. On completion, the reaction mixture was filtered and concentrated in vacuo to get the crude residue. The crude product was purified by reversed-phase HPLC (0.1% FA condition) to give the title compound (240 mg, 55% yield) as yellow solid. LC-MS (ESI⁺) m/z 417.2. (M+H)⁺.

Step 2—(R)-1-(4-(4-(6-methyl-4-oxo-4,5,6,7-tetrahydro-1H-pyrrolo[3,2-c]pyridin-2-yl)pyridin-2-yl)phenyl)piperidine-4-carbaldehyde. To a solution of (R)-2-(2-(4-(4-(dimethoxymethyl)piperidin-1-yl)phenyl)pyridin-4-yl)-6-methyl-6,7-dihydro-1H-pyrrolo[3,2-c]pyridin-4(5H)-one (80 mg, 174 umol) was added FA (174 umol, 1 mL) at 25° C., then the mixture was stirred at 25° C. for 1 hr. On completion, the reaction mixture was filtered and concentrated in vacuo to give the title compound (80 mg) as a yellow oil. LC-MS (ESI⁺) m/z 415.0. (M+H)⁺.

### Tert-butyl 4-(4-methyl-5-(4,4,5,5-tetramethyl-1,3,2-dioxaborolan-2-yl)pyridin-2-yl)piperazine-1-carboxylate (Intermediate DW)

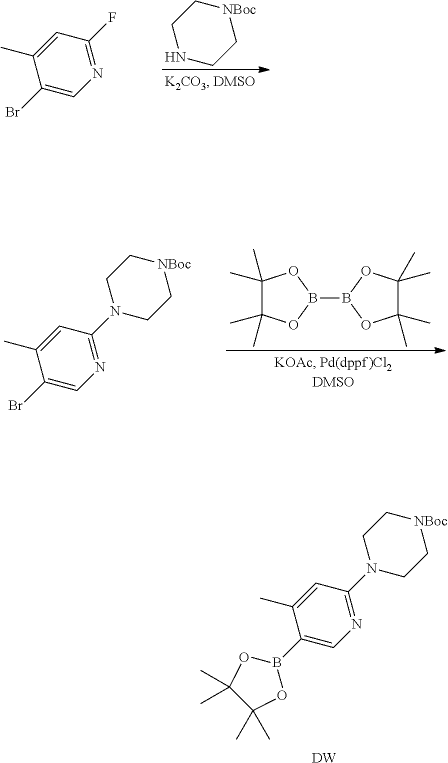

Step 1—Tert-butyl 4-(5-bromo-4-methylpyridin-2-yl)piperazine-1-carboxylate. To a solution of 5-bromo-2-fluoro-4-methylpyridine (5 g, 26.3 mmol, CAS #864830-16-0) in DMSO (100 mL) was added K₂CO₃ (10.9 g, 79.0 mmol) and tert-butyl piperazine-1-carboxylate (5.88 g, 31.6 mmol), then the mixture was stirred at 100° C. for 3 hrs. On completion, the reaction mixture was quenched by addition of H₂O (200 mL) and then filtered. The filter cake was concentrated dried reduced pressure to give the title compound (10 g) as a white solid. LC-MS (ESI⁺) m/z 357.8 (M+H)⁺.

Step 2—Tert-butyl 4-(4-methyl-5-(4,4,5,5-tetramethyl-1,3,2-dioxaborolan-2-yl)pyridin-2-yl)piperazine-1-carboxylate. A mixture of tert-butyl 4-(5-bromo-4-methylpyridin-2-yl)piperazine-1-carboxylate (5 g, 14.0 mmol), 4,4,4′,4′,5,5,5′,5′-octamethyl-2,2′-bi(1,3,2-dioxaborolane) (10.7 g, 42.1 mmol), KOAc (4.13 g, 42.1 mmol), Pd(dppf)Cl₂ (1.03 g, 1.40 mmol) in DMSO (150 mL) was degassed and purged with N₂ three times. Then the mixture was stirred at 100° C. for 2 hrs under N₂ atmosphere. On completion, the reaction mixture was added H₂O (200 mL) and then filtered. The filter cake was dried under reduced pressure to give the title compound (10 g) as a white solid. LC-MS (ESI⁺) m/z 404.2 (M+H)⁺.

### (R)-10-methyl-3-(4-methyl-6-(piperazin-1-yl)pyridin-3-yl)-9,10,11,12-tetrahydro-8H-[1,4]diazepino[5′,6′:4,5]thieno[3,2-f]quinolin-8-one (Intermediate DX)

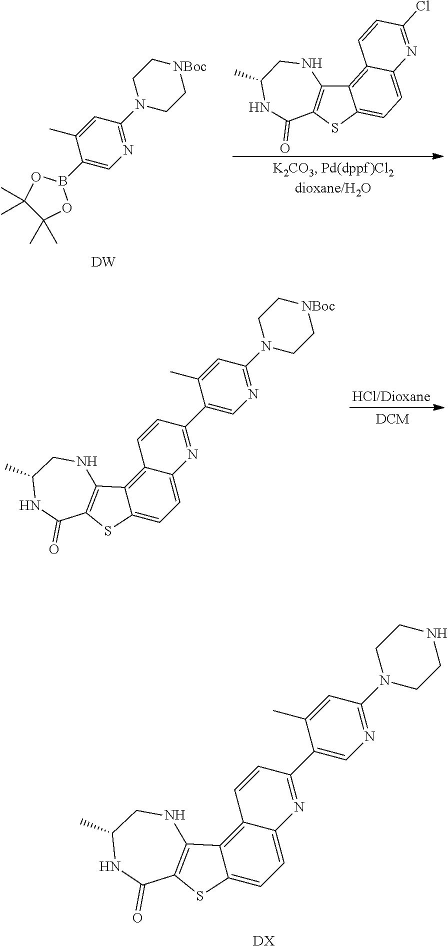

Step 1—(R)-tert-butyl 4-(4-methyl-5-(10-methyl-8-oxo-9,10,11,12-tetrahydro-8H-[1,4]diazepino[5′,6′:4,5]thieno[3,2-f]quinolin-3-yl)pyridin-2-yl)piperazine-1-carboxylate. A mixture of (R)-3-chloro-10-methyl-9,10,11,12-tetrahydro-8H-[1,4]diazepino[5′,6′:4,5]thieno[3,2-f]quinolin-8-one (1.5 g, 4.72 mmol, Intermediate N), tert-butyl 4-(4-methyl-5-(4,4,5,5-tetramethyl-1,3,2-dioxaborolan-2-yl)pyridin-2-yl)piperazine-1-carboxylate (2.28 g, 5.66 mmol, Intermediate DW), K₂CO₃ (1.96 g, 14.2 mmol), and Pd(dppf)Cl₂ (345 mg, 472 umol) in dioxane (30 mL) and H₂O (5 mL) was degassed and purged with N₂ three times. Then the mixture was stirred at 80° C. for 2 hrs under N₂ atmosphere. On completion, the reaction mixture was partitioned between EA (30 mL) and H₂O (60 mL). The organic phase was separated, washed with brine (10 mL×3), dried over Na₂SO₄, filtered and the filtrate was concentrated under reduced pressure to give a residue. The residue was purified by column chromatography (SiO₂, Petroleum ether/Ethyl acetate=5/1 to 1/1) to give the title compound (1 g, 32% yield) as a white solid. LC-MS (ESI+) m/z 559.5 (M+H)⁺.

Step 2—(R)-10-methyl-3-(4-methyl-6-(piperazin-1-yl)pyridin-3-yl)-9,10,11,12-tetrahydro-8H-[1,4]diazepino[5′,6′:4,5]thieno[3,2-f]quinolin-8-one. To a solution of (R)-tert-butyl 4-(4-methyl-5-(10-methyl-8-oxo-9,10,11,12-tetrahydro-8H-[1,4]diazepino[5′,6′:4,5]thieno[3,2-f]quinolin-3-yl)pyridin-2-yl)piperazine-1-carboxylate (300 mg, 536 umol) in DCM (3 mL) was added HCl/dioxane (4 M, 134 uL). The mixture was stirred at 25° C. for 1.5 hrs. On completion, the reaction mixture was concentrated under reduced pressure to give the title compound (240 mg, HCl) as a green solid. LC-MS (ESI+) m/z 459.2 (M+H)⁺.

### (1-(4-(4,4,5,5-tetramethyl-1,3,2-dioxaborolan-2-yl)phenyl)piperidin-4-yl)methanol (Intermediate DY)

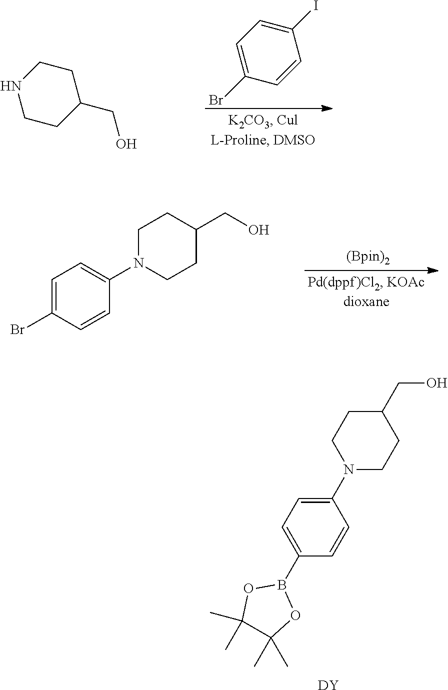

Step 1—(1-(4-Bromophenyl)piperidin-4-yl)methanol. A mixture of 1-bromo-4-iodobenzene (10 g, 35.3 mmol, CAS #589-87-7), 4-piperidylmethanol (4.07 g, 35.3 mmol, CAS #6457-49-4), K₂CO₃ (9.77 g, 70.7 mmol), CuI (1.35 g, 7.07 mmol) and L-proline (1.63 g, 14.1 mmol) in DMSO (200 mL) was degassed and purged with N₂ three times. Then the mixture was stirred at 90° C. for 12 hrs under N₂ atmosphere. On completion, the reaction mixture was diluted with H₂O (500 mL) and extracted with EA (500 mL×3). The combined organic layers were washed with brine (300 mL×3), dried over Na₂SO₄, filtered and the filtrate was concentrated under reduced pressure to give a residue. The residue was purified by column chromatography (SiO₂, Petroleum ether/Ethyl acetate=10/1 to 3/1) to give the title compound (3.5 g, 37% yield) as a white solid. LC-MS (ESI⁺) m/z 269.9 (M+H)⁺.

Step 2 (1-(4-(4,4,5,5-tetramethyl-1,3,2-dioxaborolan-2-yl)phenyl)piperidin-4-yl)methanol. A mixture of [1-(4-bromophenyl)-4-piperidyl]methanol (2 g, 7.40 mmol), 4,4,5,5-tetramethyl-2-(4,4,5,5-tetramethyl-1,3,2-dioxaborolan-2-yl)-1,3,2-dioxaborolane (3.76 g, 14.8 mmol), KOAc (2.18 g, 22 mmol), Pd(dppf)Cl₂ (541 mg, 740 umol) in DMSO (40 mL) was degassed and purged with N₂ three times, and then the mixture was stirred at 80° C. for 2 hrs under N₂ atmosphere. On completion, the reaction mixture was diluted with H₂O (150 mL) and extracted with EA (100 mL×5). The combined organic layers were washed with brine (100 mL×3), dried over Na₂SO₄, filtered and concentrated under reduced pressure to give a residue. The residue was purified by column chromatography (SiO₂, Petroleum ether/Ethyl acetate=1/0 to 1/1) to give the title compound (1.8 g, 66% yield) as a white solid. LC-MS (ESI⁺) m/z 318.0 (M+H)⁺.

### (R)-1-(4-(10,12-dimethyl-8-oxo-9,10,11,12-tetrahydro-8H-[1,4]diazepino[5′,6′:4,5]thieno[3,2-f]quinolin-3-yl)phenyl)piperidine-4-carbaldehyde (Intermediate DZ)

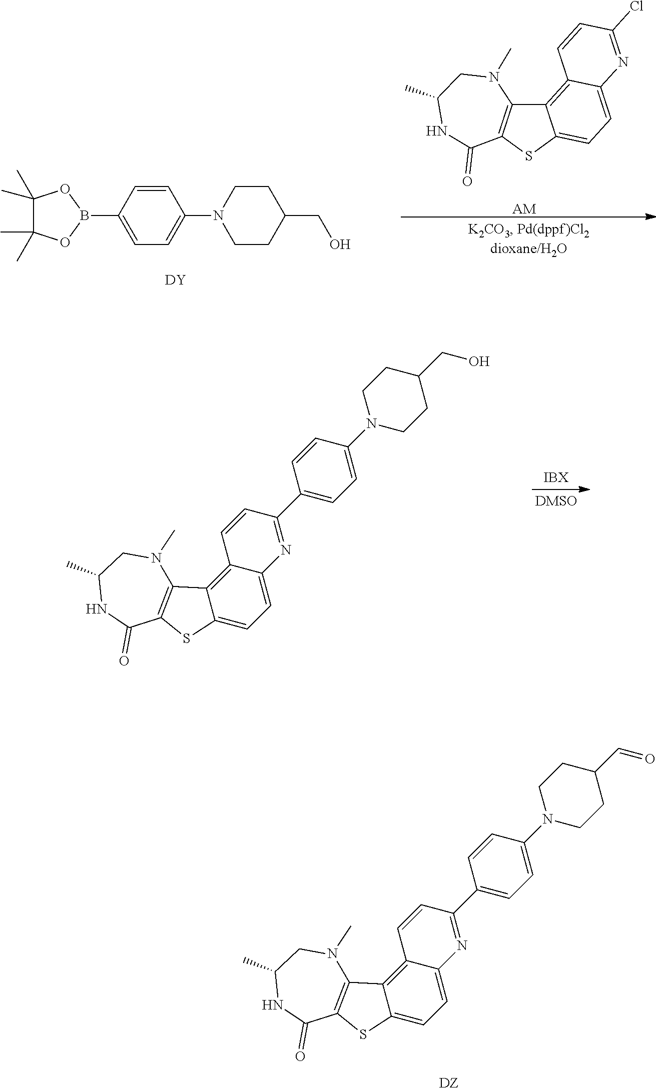

Step 1—(R)-3-(4-(4-(hydroxymethyl)piperidin-1-yl)phenyl)-10,12-dimethyl-9,10,11,12-tetrahydro-8H-[1,4]diazepino[5′,6′:4,5]thieno[3,2-f]quinolin-8-one. A mixture of (15R)-5-chloro-15,17-dimethyl-11-thia-6,14,17-triazatetracyclo[8.8.0.0^{2,7}.0^{12,18}]octadeca-1,3,5,7,9,12(18)-hexaen-13-one (150 mg, 452 umol, Intermediate AM), [1-[4-(4,4,5,5-tetramethyl-1,3,2-dioxaborolan-2-yl)phenyl]-4-piperidyl]methanol (186 mg, 587 umol, Intermediate DY), K₂CO₃ (187 mg, 1.36 mmol), Pd(dppf)Cl₂ (49.6 mg, 67.8 umol) in dioxane (3 mL) and H₂O (1 mL) was degassed and purged with N₂ three times. Then the mixture was stirred at 80° C. for 2 hrs under N₂ atmosphere. On completion, the reaction mixture was diluted with H₂O (20 mL) and extracted with EA (25 mL×4). The combined organic layers were dried over Na₂SO₄, filtered and concentrated under reduced pressure to give a residue. The residue was purified by column chromatography (SiO₂, Petroleum ether/Ethyl acetate=5/1 to 0/1) to give the title compound (200 mg, 52% yield) as a yellow solid. LC-MS (ESI⁺) m/z 487.4 (M+H)⁺.

Step 2—(R)-1-(4-(10,12-dimethyl-8-oxo-9,10,11,12-tetrahydro-8H-[1,4]diazepino[5′,6′:4,5]thieno[3,2-f]quinolin-3-yl)phenyl)piperidine-4-carbaldehyde. To a solution of (15R)-5-[4-[4-(hydroxymethyl)-1-piperidyl]phenyl]-15,17-dimethyl-11-thia-6,14,17-triazatetracyclo[8.8.0.0^{2,7}.0^{12,18}]octadeca-1,3,5,7,9,12(18)-hexaen-13-one (100 mg, 137 umol) in DMSO (2 mL) was added IBX (77.1 mg, 275 umol). The mixture was stirred at 0-25° C. for 3 hrs. On completion, the reaction mixture was diluted with H₂O (15 mL) and extracted with EA (10 mL×5). The combined organic layers were dried over Na₂SO₄, filtered and concentrated under reduced pressure to give the title compound (72 mg) was obtained as a yellow solid. LC-MS (ESI⁺) m/z 485.2 (M+H)⁺.

### 2-(4-(((tert-butyldimethylsilyl)oxy)methyl)piperidin-1-yl)-5-(4,4,5,5-tetramethyl-1,3,2-dioxaborolan-2-yl)pyridine (Intermediate EA)

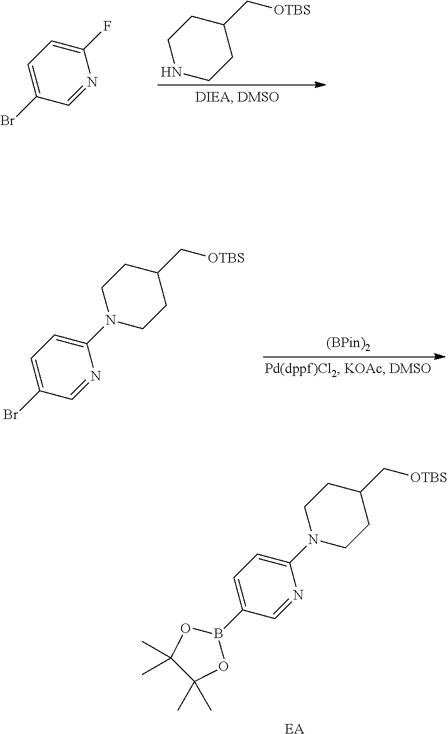 [SMILES: Fc1ccc(Br)cn1 ; CC(C)(C)[Si](C)(C)OCC1CCNCC1 ; CC(C)(C)[Si](C)(C)OCC1CCN(c2ccc(Br)cn2)CC1 ; CC1(C)OB(c2ccc(N3CCC(CO[Si](C)(C)C(C)(C)C)CC3)nc2)OC1(C)C ; "EA"]

Step 1—5-Bromo-2-(4-(((tert-butyldimethylsilyl)oxy)methyl)piperidin-1-yl)pyridine. To a solution of 5-bromo-2-fluoro-pyridine (2 g, 11.3 mmol, CAS #766-11-0) and tert-butyl-dimethyl-(4-piperidylmethoxy)silane (2.87 g, 12.5 mmol, CAS #204580-41-6) in DMSO (30 mL) was added DIEA (4.41 g, 34.1 mmol). Then the mixture was stirred at 100° C. for 3 hrs. On completion, the reaction mixture was diluted with H₂O (20 mL) and extracted with ethyl acetate (30 mL×4). The combined organic layers were washed with brine 60 mL (20 mL×3), dried over Na₂SO₄, filtered and the filtrate was concentrated under reduced pressure to give a residue. The residue was purified by column chromatography (SiO₂, Petroleum ether/Ethyl acetate=1/0 to 10/1) to give the title compound (1.6 g, 35% yield) as a white oil. LC-MS (ESI⁺) m/z 385.0 (M+H)⁺.

Step 2—2-(4-(((Tert-butyldimethylsilyl)oxy)methyl)piperidin-1-yl)-5-(4,4,5,5-tetramethyl-1,3,2-dioxaborolan-2-yl)pyridine. A mixture of [1-(5-bromo-2-pyridyl)-4-piperidyl]methoxy-tert-butyl-dimethyl-silane (1.1 g, 2.85 mmol), 4,4,5,5-tetramethyl-2-(4,4,5,5-tetramethyl-1,3,2-dioxaborolan-2-yl)-1,3,2-dioxaborolane (1.81 g, 7.14 mmol), KOAc (840 mg, 8.56 mmol), and Pd(dppf)Cl₂ (209 mg, 285 umol) in DMSO (30 mL) was degassed and purged with N₂ three times. Then the mixture was stirred at 80° C. for 2 hrs under N₂ atmosphere. On completion, the reaction mixture was diluted with H₂O (20 mL) and extracted with Ethyl acetate (30 mL×5). The combined organic layers were washed with brine (30 mL×3), dried over Na₂SO₄, filtered and the filtrate was concentrated under reduced pressure to give a residue. The residue was purified by column chromatography (SiO₂, Petroleum ether/Ethyl acetate=5/1 to 0/1) to give a title compound (1.2 g, 84% yield) as a white oil. LC-MS (ESI⁺) m/z 433.6 (M+H)⁺

### 1-(4-Methoxybenzyl)-2,6-dioxopiperidin-3-yl trifluoromethanesulfonate (Intermediate EB)

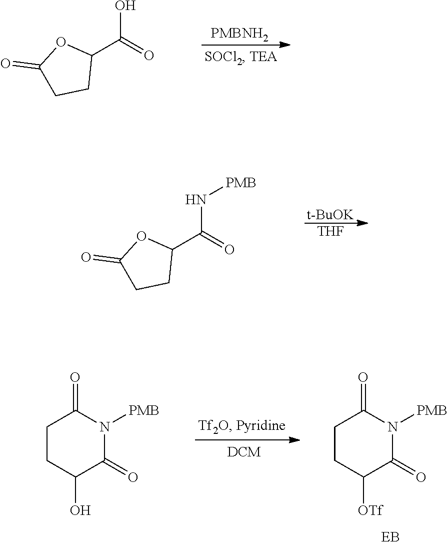

Step 1—N-(4-methoxybenzyl)-5-oxotetrahydrofuran-2-carboxamide. To 5-oxotetrahydrofuran-2-carboxylic acid (50 g, 384 mmol, CAS #4344-84-7) was added SOCl₂ (103 g, 865 mmol, 62.73 mL) at 0° C. The mixture was then stirred at 85° C. for 3 hrs. Then the reaction mixture was concentrated under reduced pressure to give a residue. To a solution of the residue in DCM (400 mL) was added TEA (77.8 g, 769 mmol, 107 mL) at 0° C., and then (4-methoxyphenyl)methanamine (42.2 g, 307 mmol, CAS #2393-23-9) was added. The mixture was stirred at 20° C. for 3 hrs. On completion the reaction mixture was concentrated under reduced pressure to give a residue. The residue was purified by column chromatography (SiO₂, Petroleum ether/Ethyl acetate=10/1 to 1/1) to give the title compound (25 g, 25% yield) as a yellow solid. LC-MS (ESI⁺) m/z 272.1 (M+Na)⁺.

Step 2—3-Hydroxy-1-(4-methoxybenzyl)piperidine-2,6-dione. To a solution of N-(4-methoxybenzyl)-5-oxotetrahydrofuran-2-carboxamide (15 g, 60.2 mmol) in THF (200 mL) was added t-BuOK (72 mL, 1 M) at −78° C. slowly. The mixture was stirred at −40° C. for 1 hr. On completion the reaction mixture was quenched by aqueous NH₄Cl (200 mL) at 0° C., then was diluted with H₂O (200 mL) and extracted with EA (200 mL×3). The combined organic layers were dried over Na₂SO₄, filtered and concentrated under reduced pressure to give a residue. The residue was purified by column chromatography (SiO₂, Petroleum ether/Ethyl acetate=10/1 to 1/1) the title compound (12 g, 78% yield) as a yellow solid. LC-MS (ESI⁺) m/z 250.1 (M+H)⁺.

Step 3—1-(4-Methoxybenzyl)-2,6-dioxopiperidin-3-yl trifluoromethanesulfonate. To a solution of 3-hydroxy-1-(4-methoxybenzyl)piperidine-2,6-dione (12 g, 48.1 mmol) in DCM (130 mL) was added pyridine (7.62 g, 96.3 mmol) and Tf₂O (20.4 g, 72.2 mmol), The mixture was stirred at 0° C. for 1 hr. On completion, the reaction mixture was concentrated under reduced pressure to give a residue. The residue was purified column chromatography (SiO₂, Petroleum ether/Ethyl acetate=10/1 to 3/1) to give the title compound (15 g, 82% yield) as a yellow oil. ¹H NMR (400 MHz, CHLOROFORM-d) 6=7.26 (d, J=8.8 Hz, 2H), 6.74 (d, J=8.8 Hz, 2H), 5.27-5.17 (m, 1H), 4.81 (s, 2H), 3.70 (s, 3H), 2.95-2.83 (m, 1H), 2.71-2.59 (m, 1H), 2.37-2.17 (m, 2H).

### 7-bromo-6-methoxy-1-methyl-1H-benzo[d]imidazol-2(3H)-one (Intermediate EC)

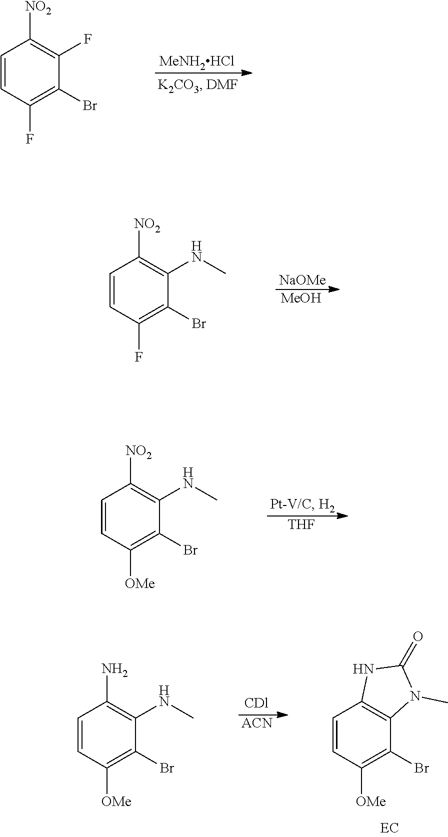

Step 1—2-Bromo-3-fluoro-N-methyl-6-nitroaniline. To a solution of 2-bromo-1,3-difluoro-4-nitrobenzene (50 g, 210 mmol) in DMF (500 mL) was added K₂CO₃ (87 g, 630 mmol) and methanamine hydrochloride (14.2 g, 210 mmol). The mixture was stirred at 25° C. for 16 hrs. On completion, the reaction mixture was filtered under reduced pressure to give a filtrate. The filtrate was diluted with H₂O (1000 mL) and extracted with EA (500 mL×3). The combined organic layers were washed with (500 mL×3), dried over Na₂SO₄, filtered and concentrated under reduced pressure to give a residue. The residue was purified column chromatography (SiO₂, Petroleum ether) to give the title compound (42 g, 79% yield) as a yellow solid. ¹H NMR (400 MHz, DMSO-d₆) δ=7.96-7.83 (m, 1H), 6.85-6.71 (m, 2H), 2.77 (d, J=5.2 Hz, 3H).

Step 2—2-Bromo-3-methoxy-N-methyl-6-nitroaniline. To a solution of 2-bromo-3-fluoro-N-methyl-6-nitroaniline (20 g, 80 mmol) in MeOH (400 mL) was added MeONa (5 M, 80 mL). The mixture was then stirred at 80° C. for 2 hrs. On completion, the reaction mixture was filtered under reduced pressure to give a filtrate. The filtrate was diluted with H₂O (1000 mL) and extracted with EA (500 mL×3). The combined organic layers were washed with (500 mL×3), dried over Na₂SO₄, filtered and concentrated under reduced pressure to give the title product (20 g). LC-MS (ESI⁺) m/z 260.9 (M+H)⁺.

Step 3—6-Bromo-5-methoxy-N1-methylbenzene-1,2-diamine. To a solution of 2-bromo-3-methoxy-N-methyl-6-nitroaniline (20 g, 76.61 mmol) in THF (500 mL) was added Pt-V/C (3.00 g, 11.5 mmol) under N₂. The suspension was degassed under vacuum and purged with H₂ several times. The mixture was stirred under H₂ (45 psi) at 25° C. for 12 hrs. On completion, the reaction mixture was filtered under reduced pressure to give the title compound (18 g) as a yellow solid. LC-MS (ESI⁺) m/z 231.1 (M+H)⁺.

Step 4—7-Bromo-6-methoxy-1-methyl-1H-benzo[d]imidazol-2(3H)-one. To a solution of 6-bromo-5-methoxy-N1-methylbenzene-1,2-diamine (17.5 g, 75.7 mmol) in ACN (250 mL) was added CDI (36.84 g, 227.2 mmol). The mixture was stirred at 85° C. for 12 hrs. On completion, the reaction mixture was concentrated under reduced pressure to give a residue. To the residue was added H₂O (500 mL), and then filtered to give the title compound (20 g) as a brown solid. LC-MS (ESI⁺) m/z 257.0 (M+H)⁺.

### 1-(5-Methoxy-1-(1-(4-methoxybenzyl)-2,6-dioxopiperidin-3-yl)-3-methyl-2-oxo-2,3-dihydro-1H-benzo[d]imidazol-4-yl)piperidine-4-carbaldehyde (Intermediate ED)

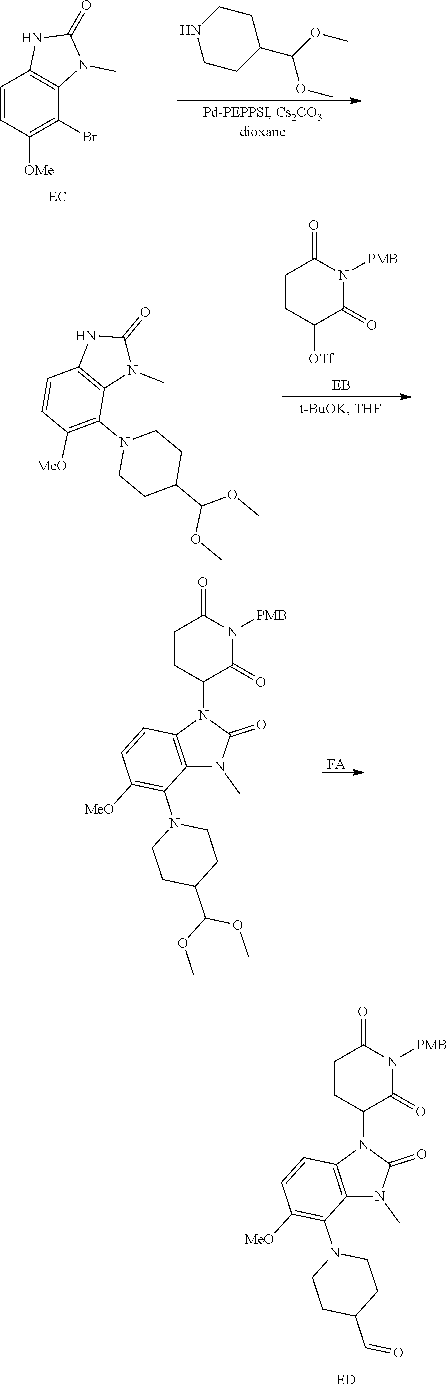

Step 1—7-(4-(Dimethoxymethyl)piperidin-1-yl)-6-methoxy-1-methyl-1H-benzo[d]imidazol-2(3H)-one. To a solution of 4-bromo-5-methoxy-3-methyl-1H-benzimidazol-2-one (1 g, 4 mmol, Intermediate EC) and 4-(dimethoxymethyl)piperidine (929 mg, 5.83 mmol) in dioxane (15 mL) was added tBuONa (1.12 g, 11.7 mmol) and 1,3-bis[2,6-bis(1-propylbutyl)phenyl]-4,5-dichloro-2H-imidazol-1-ium-2-ide;3-chloropyridine dichloropalladium (378 mg, 389 umol). The mixture was stirred at 120° C. for 12 hrs under N₂ and microwave. On completion, the reaction mixture was quenched by addition of aqueous NH4C₁ (50 mL) at 0° C., and then diluted with H₂O (50 mL) and extracted with EA (50 mL×3). The combined organic layers were washed with aqueous NaCl mL (50 mL×3), dried over Na₂SO₄, filtered and concentrated under reduced pressure to give a residue. The residue was purified by column chromatography (SiO₂, Petroleum ether/Ethyl acetate=10/1 to 1/1) to give the title compound (500 mg, 19% yield) as a yellow solid. LC-MS (ESI⁺) m/z 336.1 (M+H)⁺.

Step 2—3-(4-(4-(Dimethoxymethyl)piperidin-1-yl)-5-methoxy-3-methyl-2-oxo-2,3-dihydro-1H-benzo[d]imidazol-1-yl)-1-(4-methoxybenzyl)piperidine-2,6-dione. To a solution of 7-(4-(dimethoxymethyl)piperidin-1-yl)-6-methoxy-1-methyl-1H-benzo[d]imidazol-2(3H)-one (270 mg, 805 umol) in THF (10 mL) was added tBuOK (180 mg, 1.61 mmol) stirred at 0° C. for 0.5 hrs, and then 1-(4-methoxybenzyl)-2,6-dioxopiperidin-3-yl trifluoromethanesulfonate (614 mg, 1.61 mmol, Intermediate EB) was added. The mixture was stirred at 25° C. for 11.5 hrs. On completion, the reaction mixture was quenched with aqueous NH₄Cl (50 mL) at 0° C., and then diluted with H₂O (50 mL) and extracted with EA (50 mL×3). The combined organic layers were washed with aqueous NaCl (50 mL×3), dried over Na₂SO₄, filtered and concentrated under reduced pressure to give a residue. The residue was purified by column chromatography (SiO₂, Petroleum ether/Ethyl acetate=10/1 to 1/1) to give the title compound (150 mg, 27% yield) as a yellow solid. LC-MS (ESI⁺) m/z 567.4 (M+H)⁺

Step 3—1-(5-Methoxy-1-(1-(4-methoxybenzyl)-2,6-dioxopiperidin-3-yl)-3-methyl-2-oxo-2,3-dihydro-1H-benzo[d]imidazol-4-yl)piperidine-4-carbaldehyde. 3-(4-(4-(dimethoxymethyl)piperidin-1-yl)-5-methoxy-3-methyl-2-oxo-2,3-dihydro-1H-benzo[d]imidazol-1-yl)-1-(4-methoxybenzyl)piperidine-2,6-dione (140 mg, 247 umol) was added to HCOOH (2 mL). The mixture was stirred at 40° C. for 10 min. On completion, the reaction mixture was concentrated under reduced pressure to give the title compound (120 mg) as a brown solid. LC-MS (ESI⁺) m/z 521.3 (M+H)⁺.

### (R)-1-(5-(10-methyl-8-oxo-9,10,11,12-tetrahydro-8H-[1,4]diazepino[5′,6′:4,5]thieno[3,2-f]quinolin-3-yl)pyridin-2-yl)piperidine-4-carbaldehyde (Intermediate EE)

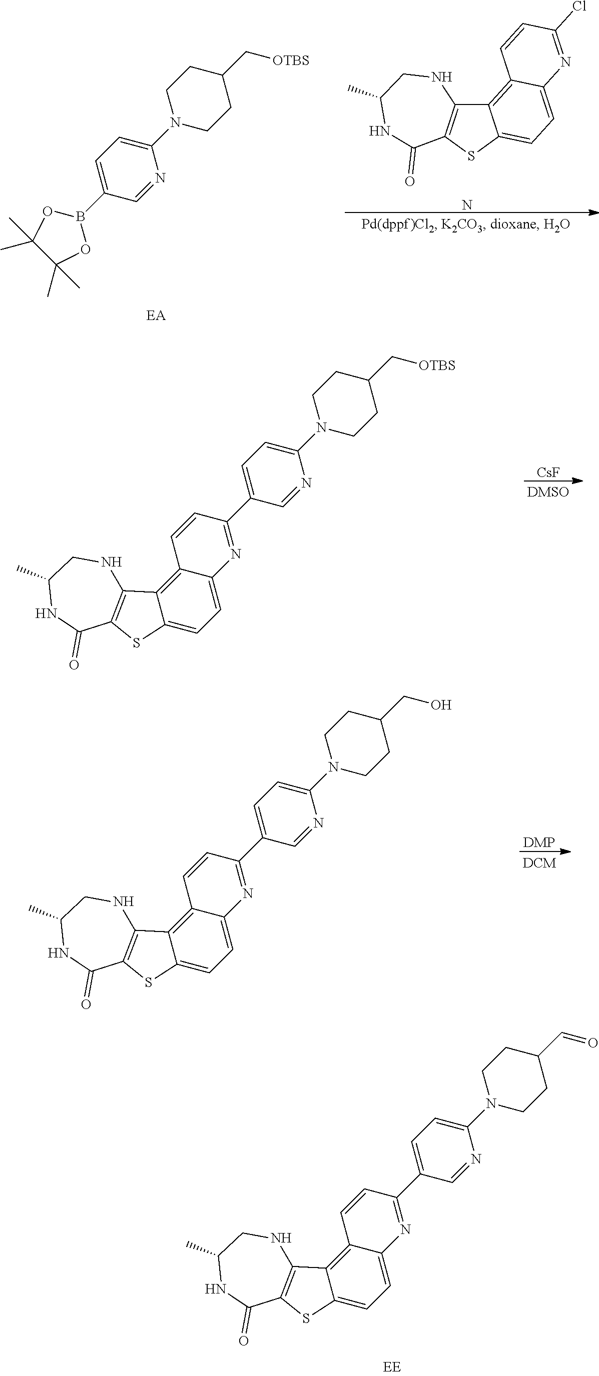

Step 1—(R)-3-(6-(4-(((tert-butyldimethylsilyl)oxy)methyl)piperidin-1-yl)pyridin-3-yl)-10-methyl-9,10,11,12-tetrahydro-8H-[1,4]diazepino[5′,6′:4,5]thieno[3,2-f]quinolin-8-one. A mixture of tert-butyl-dimethyl-[[1-[15-(4,4,5,5-tetramethyl-1,3,2-dioxaborolan-2-yl)-2-pyridyl]-4-piperidyl]methoxy]silane (530 mg, 1.23 mmol, Intermediate EA), (15R)-5-chloro-15-methyl-11-thia-6,14,17-triazatetracyclo[8.8.0.0^{2,7}.0^{12,18}]octadeca-1,3,5,7,9,12(18)-hexaen-13-one (300 mg, 944 umol, Intermediate N), K₂CO₃ (391 mg, 2.83 mmol), and Pd(dppf)Cl₂ (69.1 mg, 94.4 umol) in dioxane (12 mL) and H₂O (3 mL) was degassed and purged with N₂ three times. Then the mixture was stirred at 80° C. for 2 hrs under N₂ atmosphere. On completion, the reaction mixture was quenched by addition of H₂O (15 mL), and extracted with ethyl acetate (25 mL×4). The combined organic layers were dried over Na₂SO₄, filtered and the filtrate was concentrated under reduced pressure to give a residue. The residue was purified by column chromatography (SiO₂, Petroleum ether/Ethyl acetate=3/1 to 0/1) to give a title compound (260 mg, 41% yield) as a yellow solid. LC-MS (ESI⁺) m/z 588.2 (M+H)⁺.

Step 2—(R)-3-(6-(4-(hydroxymethyl)piperidin-1-yl)pyridin-3-yl)-10-methyl-9,10,11,12-tetrahydro-8H-[1,4]diazepino[5′,6′:4,5]thieno[3,2-f]quinolin-8-one. To a solution of (15R)-5-[6-[4-[[tert-butyl(dimethyl)silyl]oxymethyl]-1-piperidyl]-3-pyridyl]-15-methyl-11-thia-6,14,17-triazatetracyclo[8.8.0.02,7.012,18]octadeca-1,3,5,7,9,12(18)-hexaen-13-one (210 mg, 357 umol) in DMSO (3 mL) was added CsF (542 mg, 3.57 mmol). The mixture was stirred at 50° C. for 2 hrs. On completion, the reaction mixture was diluted with H₂O (15 mL) and extracted with ethyl acetate (25 mL×4). The combined organic layers were washed with brine (15 mL×4), dried over Na₂SO₄, filtered and the filtrate was concentrated under reduced pressure to give a residue. The residue was purified by column chromatography (SiO₂, Petroleum ether/Ethyl acetate=3/1 to 0/1, Dichloromethane:Methanol=10:1) to give title compound (70 mg, 35% yield) as a white solid. LC-MS (ESI⁺) m/z 474.2 (M+H)⁺.

Step 3—(R)-1-(5-(10-methyl-8-oxo-9,10,11,12-tetrahydro-8H-[1,4]diazepino[5′,6′:4,5]thieno[3,2-f]quinolin-3-yl)pyridin-2-yl)piperidine-4-carbaldehyde. To a solution of (15R)-5-[6-[4-(hydroxymethyl)-1-piperidyl]-3-pyridyl]-15-methyl-11-thia-6,14,17-triazatetracyclo[8.8.0.0^{2,7}.0^{12,18}]octadeca-1,3,5,7,9,12(18)-hexaen-13-one (70 mg, 147 umol) in DCM (2 mL) was added DMP (81.5 mg, 192 umol). The mixture was stirred at 0-25° C. for 3 hrs. On completion, the reaction mixture was quenched with sat. Na₂S₂O₃ (15 mL) at 25° C., and then diluted with H₂O (10 mL) and extracted with DCM (20 mL×3), then extracted with ethyl acetate (20 mL×3). The combined organic layers were dried over Na₂SO₄, filtered and the filtrate was concentrated under reduced pressure to give the title compound (60 mg) as a white solid. LC-MS (ESI⁺) m/z 472.3 (M+H)⁺.

### Tert-butyl 4-(5-methoxy-3-methyl-2-oxo-2,3-dihydro-1H-benzo[d]imidazol-4-yl)piperazine-1-carboxylate (Intermediate EF)

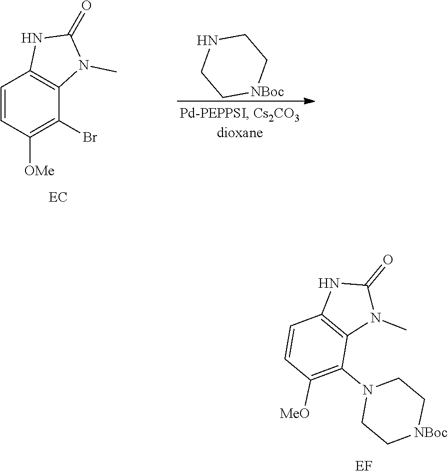 [SMILES: COc1ccc2[nH]c(=O)n(C)c2c1Br ; CC(C)(C)OC(=O)N1CCNCC1 ; COc1ccc2[nH]c(=O)n(C)c2c1N1CCN(C(=O)OC(C)(C)C)CC1]

To a solution of 4-bromo-5-methoxy-3-methyl-1H-benzimidazol-2-one (500 mg, 2 mmol, Intermediate EC) and tert-butyl piperazine-1-carboxylate (542 mg, 2.91 mmol) in dioxane (15 mL) was added tBuONa (560 mg, 5.82 mmol) and 1,3-bis[2,6-bis(1-propylbutyl)phenyl]-4,5-dichloro-2H-imidazol-1-ium-2-ide 3-chloropyridine dichloropalladium (189 mg, 194 umol). The mixture was stirred at 120° C. for 12 hrs under N₂ and microwave. On completion, the reaction mixture was quenched with aqueous NH4C₁ (50 mL) at 0° C., and then diluted with H₂O (50 mL) and extracted with EA (50 mL×3). The combined organic layers were washed with aqueous NaCl mL (50 mL×3), dried over Na₂SO₄, filtered and concentrated under reduced pressure to give a residue. The residue was purified by column chromatography (SiO₂, Petroleum ether/Ethyl acetate=10/1 to 1/1) to give the title compound (270 mg, 31% yield) as a yellow solid. LC-MS (ESI⁺) m/z 363.1 (M+H)⁺.

### 3-(5-Methoxy-3-methyl-2-oxo-4-(piperazin-1-yl)-2,3-dihydro-1H-benzo[d]imidazol-1-yl)piperidine-2,6-dione (Intermediate EG)

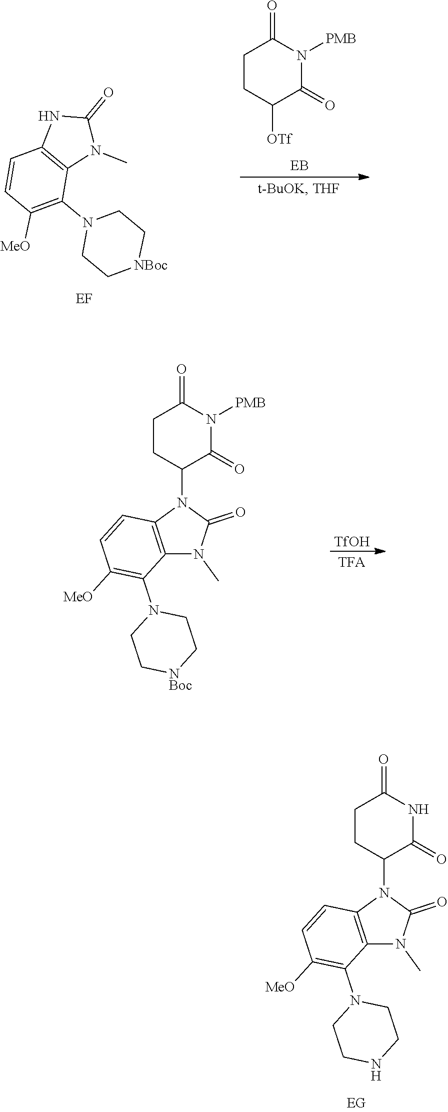

Step 1—Tert-butyl 4-(5-methoxy-1-(1-(4-methoxybenzyl)-2,6-dioxopiperidin-3-yl)-3-methyl-2-oxo-2,3-dihydro-1H-benzo[d]imidazol-4-yl)piperazine-1-carboxylate. To a solution of tert-butyl 4-(5-methoxy-3-methyl-2-oxo-2,3-dihydro-1H-benzo[d]imidazol-4-yl)piperazine-1-carboxylate (260 mg, 717 umol, Intermediate EF) in THF (10 mL) was added tBuOK (161.00 mg, 1.43 mmol) and the mixture was stirred at 0° C. for 0.5 hrs. Then 1-(4-methoxybenzyl)-2,6-dioxopiperidin-3-yl trifluoromethanesulfonate (547 mg, 1.43 mmol, Intermediate EB) was added and the mixture was stirred at 25° C. for 11.5 hrs. On completion, the reaction mixture was quenched with aqueous NH4C₁ (50 mL) at 0° C., and then diluted with H₂O (50 mL) and extracted with EA (50 mL×3). The combined organic layers were washed with aqueous NaCl (50 mL×3), dried over Na₂SO₄, filtered and concentrated under reduced pressure to give a residue. The residue was purified by column chromatography (SiO₂, Petroleum ether/Ethyl acetate=10/1 to 1/1) to give the title compound (200 mg, 40% yield) as a yellow solid. LC-MS (ESI⁺) m/z 594.3 (M+H)⁺.

Step 2—3-(5-Methoxy-3-methyl-2-oxo-4-(piperazin-1-yl)-2,3-dihydro-1H-benzo[d]imidazol-1-yl)piperidine-2,6-dione. To a solution of tert-butyl 4-(5-methoxy-1-(1-(4-methoxybenzyl)-2,6-dioxopiperidin-3-yl)-3-methyl-2-oxo-2,3-dihydro-1H-benzo[d]imidazol-4-yl)piperazine-1-carboxylate (200 mg, 337 umol) in TFA (2 mL) was added TfOH (0.2 mL). The mixture was then stirred at 70° C. for 1 hr. On completion, the reaction mixture was concentrated under reduced pressure to give a residue. The residue was purified by reversed-phase HPLC (0.1% HCl condition) to give the title compound (60 mg, 41% yield, HCl salt) as an off-white solid. LC-MS (ESI⁺) m/z 374.1 (M+H)⁺.

### (6-(4-(tert-butoxycarbonyl)piperazin-1-yl)-5-methylpyridin-3-yl)boronic acid (Intermediate EH)

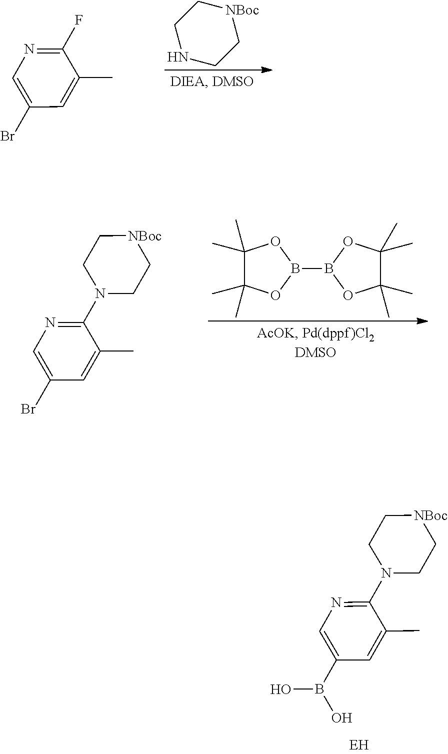

Step 1—Tert-butyl 4-(5-bromo-3-methylpyridin-2-yl)piperazine-1-carboxylate. To a solution of tert-butyl piperazine-1-carboxylate (7.35 g, 39.5 mmol, CAS #29312-98-9) in DMSO (50 mL) was added DIEA (17.0 g, 132 mmol, 22.9 mL) and 5-bromo-2-fluoro-3-methyl-pyridine (5.00 g, 26.3 mmol, CAS #57260-71-6) at 25° C. The mixture was stirred at 120° C. for 40 hrs. On completion, the reaction mixture was partitioned between EA (200 mL×3) and H₂O (200 mL). The organic phase was separated, washed with brine (200 mL×3), dried over Na₂SO₄, filtered and concentrated under reduced pressure to give a residue. The residue was purified by column chromatography (SiO₂, Petroleum ether/Ethyl acetate=1/0 to 10/1) to give the title compound (4.3 g, 44% yield) as a yellow solid. LC-MS (ESI⁺) m/z 358.0 (M+H)⁺.

Step 2—(6-(4-(Tert-butoxycarbonyl)piperazin-1-yl)-5-methylpyridin-3-yl)boronic acid. To a solution of tert-butyl 4-(5-bromo-3-methyl-2-pyridyl)piperazine-1-carboxylate (2 g, 5.61 mmol),4,4,5,5-tetramethyl-2-(4,4,5,5-tetramethyl-1,3,2-dioxaborolan-2-yl)-1,3,2-dioxaborolane (2.85 g, 11.2 mmol), AcOK (1.65 g, 16.8 mmol) and Pd(dppf)Cl₂ (410 mg, 561 umol) in dioxane (60 mL) was degassed and purged with N₂ three times. Then the mixture was stirred at 80° C. for 4 hrs under N₂ atmosphere. On completion, the reaction mixture was partitioned between EA (200 mL×3) and H₂O (200 mL). The organic phase was separated, washed with brine (200 mL×3), dried over Na₂SO₄, filtered and concentrated under reduced pressure to give a residue. The residue was purified by reversed-phase HPLC (0.1% FA condition) to give the title compound (780 mg, 38% yield) as a white solid. LC-MS (ESI+) m/z 322.0 (M+H)⁺.

### (R)-10-methyl-3-(5-methyl-6-(piperazin-1-yl)pyridin-3-yl)-9,10,11,12-tetrahydro-8H-[1,4]diazepino[5′,6′:4,5]thieno[3,2-f]quinolin-8-one (Intermediate EI)

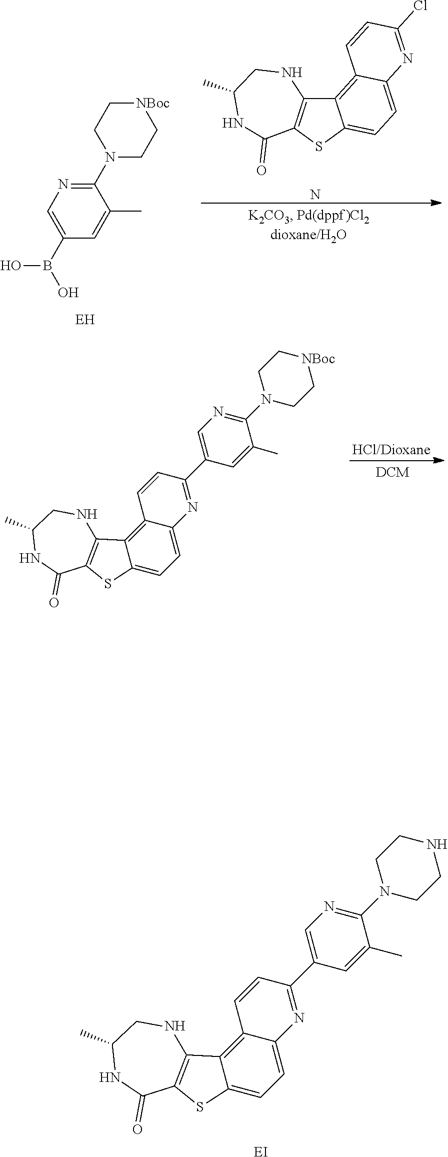

Step 1—(R)-tert-butyl 4-(3-methyl-5-(10-methyl-8-oxo-9,10,11,12-tetrahydro-8H-[1,4]diazepino[5′,6′:4,5]thieno[3,2-f]quinolin-3-yl)pyridin-2-yl)piperazine-1-carboxylate. To a solution of [6-(4-tert-butoxycarbonylpiperazin-1-yl)-5-methyl-3-pyridyl]boronic acid (400 mg, 1.25 mmol, Intermediate EH) and (15R)-5-chloro-15-methyl-1l-thia-6,14,17-triazatetracyclo[8.8.0.0^{2,7}.0^{12,18}]octadeca-1,3,5,7,9,12(18)-hexaen-13-one (400 mg, 1.26 mmol, Intermediate N) in dioxane (8 mL) and H₂O (2 mL) was added Pd(dppf)Cl₂ (228 mg, 311 umol) and K₂CO₃ (861 mg, 6.23 mmol). The mixture was degassed and purged with N₂ three times, and then the mixture was stirred at 60° C. for 1 hr under N₂ atmosphere. On completion, the reaction mixture was quenched with H₂O (100 mL) at 25° C., and then diluted with EA (100 mL). A solid precipitated formed which was filtered to give the filter cake as the title compound (1.2 g) as a yellow solid. LC-MS (ESI⁺) m/z 559.3 (M+H)⁺.

Step 2—(R)-10-methyl-3-(5-methyl-6-(piperazin-1-yl)pyridin-3-yl)-9,10,11,12-tetrahydro-8H-[1,4]diazepino[5′,6′:4,5]thieno[3,2-f]quinolin-8-one. To a solution of tert-butyl 4-[3-methyl-5-[(15R)-15-methyl-13-oxo-11-thia-6,14,17-triazatetracyclo[8.8.0.0^{2,7}.0^{12,18}]octadeca-1,3,5,7,9,12(18)-hexaen-5-yl]-2-pyridyl]piperazine-1-carboxylate (100 mg, 179 umol) in DCM (10 mL) was added HCl/dioxane (4 M, 44.8 uL). The mixture was stirred at 25° C. for 1 hr. On completion, the reaction mixture was filtered. The crude product was triturated with DCM at 25° C. for 10 min to give the title compound (120 mg, HCl) as a red solid. LC-MS (ESI⁺) m/z 459.1 (M+H)⁺.

### Tert-butyl 4-(4-(4,4,5,5-tetramethyl-1,3,2-dioxaborolan-2-yl)pyridin-2-yl)piperazine-1-carboxylate (Intermediate EJ)

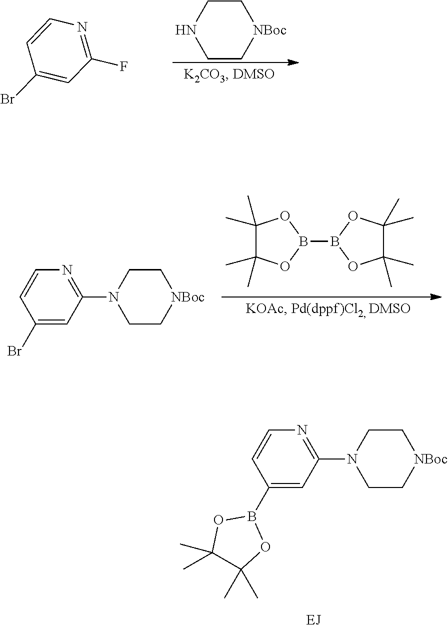

Step 1—Tert-butyl 4-(4-bromopyridin-2-yl)piperazine-1-carboxylate. To a solution of 4-bromo-2-fluoropyridine (20 g, 114 mmol, CAS #128071-98-7) in DMSO (200 mL) was added K₂CO₃ (47.1 g, 341 mmol) and tert-butyl piperazine-1-carboxylate (31.8 g, 170 mmol). The mixture was stirred at 100° C. for 3 hrs. On completion, the reaction mixture was added H₂O (200 mL) and then filtered. The filter cake was dried under reduced pressure to give the title compound (45 g) as a white solid. LC-MS (ESI+) m/z 344.1 (M+H)⁺.

Step 2—Tert-butyl 4-(4-(4,4,5,5-tetramethyl-1,3,2-dioxaborolan-2-yl)pyridin-2-yl)piperazine-1-carboxylate. To a solution of tert-butyl 4-(4-bromo-2-pyridyl)piperazine-1-carboxylate (10 g, 29.2 mmol) and 4,4,5,5-tetramethyl-2-(4,4,5,5-tetramethyl-1,3,2-dioxaborolan-2-yl)-1,3,2-dioxaborolane (33.3 g, 131 mmol) in DMSO (100 mL) was added KOAc (8.60 g, 87.6 mmol) and Pd(dppf)Cl₂ (2.14 g, 2.92 mmol). The mixture was stirred at 80° C. for 0.3 hrs. On completion, filtered and concentrated under reduced pressure to give the title compound (6 g) as a black solid. LC-MS (ESI⁺) m/z 308.1 (M+H)⁺.

### (R)-10-methyl-3-(2-(piperazin-1-yl)pyridin-4-yl)-9,10,11,12-tetrahydro-8H-[1,4]diazepino[5′,6′:4,5]thieno[3,2-f]quinolin-8-one (Intermediate EK)

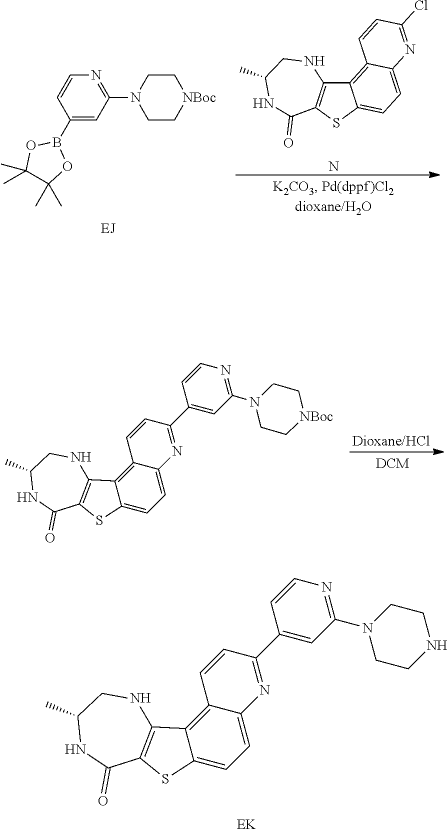

Step 1—(R)-tert-butyl 4-(4-(10-methyl-8-oxo-9,10,11,12-tetrahydro-8H-[1,4]diazepino[5′,6′:4,5]thieno[3,2-f]quinolin-3-yl)pyridin-2-yl)piperazine-1-carboxylate. To a mixture of tert-butyl 4-(4-(4,4,5,5-tetramethyl-1,3,2-dioxaborolan-2-yl)pyridin-2-yl)piperazine-1-carboxylate (3.86 g, 9.92 mmol, Intermediate EJ) in dioxane (50 mL) was added (R)-3-chloro-10-methyl-9,10,11,12-tetrahydro-8H-[1,4]diazepino[5′,6′:4,5]thieno[3,2-f]quinolin-8-one (2.1 g, 6.6 mmol, Intermediate N), K₂CO₃ (2.74 g, 19.8 mmol) in H₂O (5 mL) and Pd(dppf)Cl₂ (484 mg, 661 umol). Then the mixture was degassed and purged with N₂ three times, and then the mixture was stirred at 80° C. for 1 hr under N₂ atmosphere. On completion, the mixture filtered and concentrated under reduced pressure to give the title compound (2.7 g) as a green solid. LC-MS (ESI+) m/z 545.5 (M+H)⁺.

Step 2—(R)-10-methyl-3-(2-(piperazin-1-yl)pyridin-4-yl)-9,10,11,12-tetrahydro-8H-[1,4]diazepino[5′,6′:4,5]thieno[3,2-f]quinolin-8-one. To a solution of (R)-tert-butyl 4-(4-(10-methyl-8-oxo-9,10,11,12-tetrahydro-8H-[1,4]diazepino[5′,6′:4,5]thieno[3,2-f]quinolin-3-yl)pyridin-2-yl)piperazine-1-carboxylate (2.7 g, 5.0 mmol) in DCM (20 mL) was added dioxane/HCl (4 M, 3.72 mL), then the mixture was stirred at 25° C. for 2 hrs. On completion, the mixture was filtered and the filter cake was concentrated under reduced pressure to give the title compound (2.9 g, HCl) as a green solid. LC-MS (ESI+) m/z 445.1 (M+H)⁺.

### Tert-butyl 7-(3-(4,4,5,5-tetramethyl-1,3,2-dioxaborolan-2-yl)phenyl)-2,7-diazaspiro[3.5]nonane-2-carboxylate (Intermediate EL)

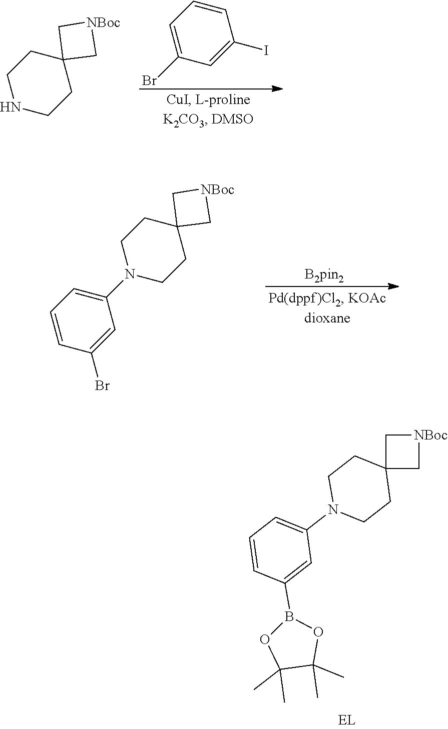 [SMILES: CC(C)(C)OC(=O)N1CC2(CCNCC2)C1 ; Brc1cccc(I)c1 ; CC(C)(C)OC(=O)N1CC2(CCN(c3cccc(Br)c3)CC2)C1 ; CC(C)(C)OC(=O)N1CC2(CCN(c3cccc(B4OC(C)(C)C(C)(C)O4)c3)CC2)C1]

Step 1—Tert-butyl 7-(3-bromophenyl)-2,7-diazaspiro[3.5]nonane-2-carboxylate. To a solution of 1-bromo-3-iodobenzene (5 g, 17.7 mmol, CAS #591-18-4) in DMSO (50 mL) was added tert-butyl 2,7-diazaspiro[3.5]nonane-2-carboxylate (4.40 g, 19.4 mmol, CAS #236406-55-6), CuI (673 mg, 3.53 mmol), L-proline (814 mg, 7.07 mmol) and K₂CO₃ (4.89 g, 35.6 mmol). Then the mixture was purged with N₂ three times, and then the mixture was stirred at 80° C. for 2 hrs under N₂ atmosphere. On completion, the mixture was quenched with H₂O (200 mL), extracted with EtOAc (100 mL×3), and washed with brine (100 mL×3). The organic phase was concentrated under reduced pressure to give the title compound (8 g) as a brown oil. LC-MS (ESI⁺) m/z 381.0 (M+H)⁺.

Step 2—Tert-butyl 7-(3-(4,4,5,5-tetramethyl-1,3,2-dioxaborolan-2-yl)phenyl)-2,7-diazaspiro[3.5]nonane-2-carboxylate. To a solution of tert-butyl 7-(3-bromophenyl)-2,7-diazaspiro[3.5]nonane-2-carboxylate (1.5 g, 3.93 mmol) in DMSO (20 mL) was added 4,4,5,5-tetramethyl-2-(4,4,5,5-tetramethyl-1,3,2-dioxaborolan-2-yl)-1,3,2-dioxaborolane (2.00 g, 7.87 mmol), Pd(dppf)Cl₂ (288 mg, 393 umol) and KOAc (1.16 g, 11.8 mmol), and the reaction was degassed and purged with N₂ three times. Then the mixture was stirred at 80° C. for 4 hrs under N₂ atmosphere. On completion, the mixture was concentrated under reduced pressure to give a residue. The residue was purified by column chromatography (SiO₂, Petroleum ether/Ethyl acetate=10/1 to 1/1) to give the title compound (800 mg, 1.87 mmol) as a white solid. LC-MS (ESI⁺) m/z 429.1 (M+H)⁺.

### (2-(4-(dimethoxymethyl)piperidin-1-yl)pyridin-4-yl)boronic acid (Intermediate EM)

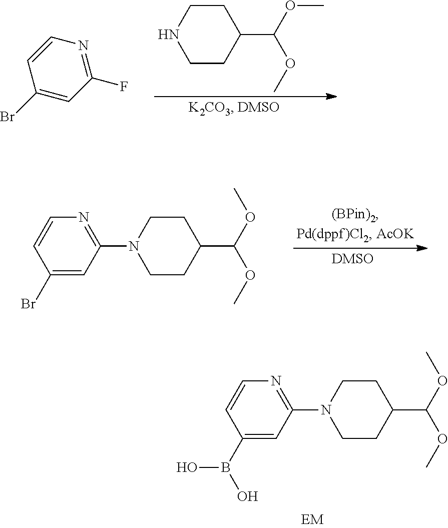 [SMILES: Fc1cc(Br)ccn1 ; COC(OC)C1CCNCC1 ; COC(OC)C1CCN(c2cc(Br)ccn2)CC1 ; COC(OC)C1CCN(c2cc(B(O)O)ccn2)CC1]

Step 1—4-Bromo-2-(4-(dimethoxymethyl)piperidin-1-yl)pyridine. To a solution of 4-bromo-2-fluoropyridine (4.2 g, 23.9 mmol, CAS #128071-98-7) and 4-(dimethoxymethyl)piperidine (4.56 g, 28.6 mmol, CAS #188846-83-5) in DMSO (40 mL) was added K₂CO₃ (16.5 g, 119 mmol) at 25° C., then the reaction was stirred at 100° C. for 2 hrs. On completion, the reaction mixture was quenched with water (40 mL) and extracted by ethyl acetate (3×30 mL). The extracts were washed with brine (50 mL) and dried over anhydrous sodium sulfate, filtered and concentrated in vacuo to get the crude residue. The residue was purified by column chromatography (SiO₂, Petroleum ether/Ethyl acetate=20/1) to give the title compound (5.7 g, 74% yield) as white solid. LC-MS (ESI⁺) m/z 314.9. (M+H)⁺.

Step 2—(2-(4-(Dimethoxymethyl)piperidin-1-yl)pyridin-4-yl)boronic acid. To a solution of 4-bromo-2-(4-(dimethoxymethyl)piperidin-1-yl)pyridine (600 mg, 1.90 mmol) and 4,4,5,5-tetramethyl-2-(4,4,5,5-tetramethyl-1,3,2-dioxaborolan-2-yl)-1,3,2-dioxaborolane (2.18 g, 8.57 mmol) in DMSO (20 mL) was added AcOK (560 mg, 5.71 mmol) and Pd(dppf)Cl₂ (139 mg, 190 umol) at 25° C., the reaction was stirred at 100° C. for 2 hrs. On completion, the reaction mixture was quenched with water (100 mL) and extracted with ethyl acetate (3×30 mL). The extracts were washed with brine (100 mL) and dried over anhydrous sodium sulfate, filtered and concentrated in vacuo to get the crude residue. The crude product was purified by reversed-phase HPLC (0.1% FA condition) to give the title compound (900 mg, 79% yield) as white solid. LC-MS (ESI⁺) m/z 218.1. (M+H)⁺.

### (R)-1-(4-(10-methyl-8-oxo-9,10,11,12-tetrahydro-8H-[1,4]diazepino[5′,6′:4,5]thieno[3,2-f]quinolin-3-yl)pyridin-2-yl)piperidine-4-carbaldehyde (Intermediate EN)

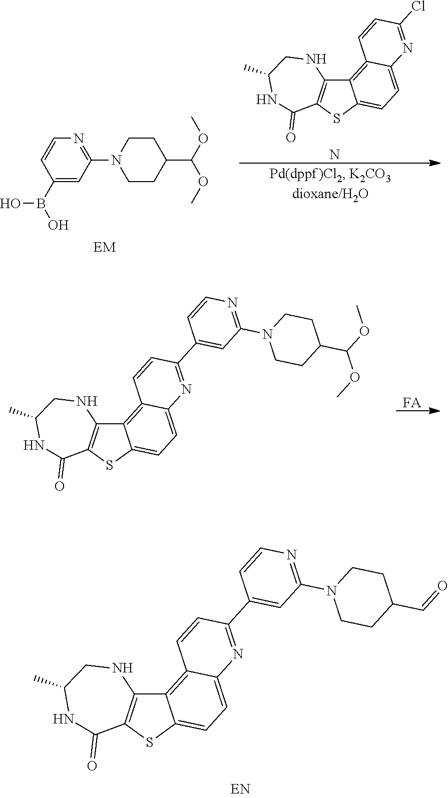

Step 1—(R)-3-(2-(4-(dimethoxymethyl)piperidin-1-yl)pyridin-4-yl)-10-methyl-9,10,11,12-tetrahydro-8H-[1,4]diazepino[5′,6′:4,5]thieno[3,2-f]quinolin-8-one. To a solution of (2-(4-(dimethoxymethyl)piperidin-1-yl)pyridin-4-yl)boronic acid (423.11 mg, 1.51 mmol, Intermediate EM) and (R)-3-chloro-10-methyl-9,10,11,12-tetrahydro-8H-[1,4]diazepino[5′,6′:4,5]thieno[3,2-f]quinolin-8-one (400 mg, 1.26 mmol, Intermediate N) in dioxane (10 mL) was added Pd(dppf)Cl₂ (92.1 mg, 126 umol) at 25° C., then was added K₂CO₃ (2 M, 1.89 mL) in H₂O (2 mL) at 25° C. Then the mixture was stirred at 80° C. for 2 hrs. On completion, the reaction mixture was filtered and concentrated in vacuo to get the crude residue. The residue was purified by column chromatography (SiO₂, Dichloromethane:Methanol=25/1) to give the title compound (570 mg, 73% yield) as yellow solid. LC-MS (ESI⁺) m/z 518.2. (M+H)⁺.

Step 2—(R)-1-(4-(10-methyl-8-oxo-9,10,11,12-tetrahydro-8H-[1,4]diazepino[5′,6′:4,5]thieno[3,2-f]quinolin-3-yl)pyridin-2-yl)piperidine-4-carbaldehyde. To a solution of (R)-3-(2-(4-(dimethoxymethyl)piperidin-1-yl)pyridin-4-yl)-10-methyl-9,10,11,12-tetrahydro-8H-[1,4]diazepino[5′,6′:4,5]thieno[3,2-f]quinolin-8-one (100 mg, 193.18 umol) in HCOOH (9.28 mg, 193 umol), then the reaction was stirred at 25° C. for 1 hr. On completion, the reaction mixture was filtered and concentrated in vacuo to give the title compound (140 mg) as brown oil. LC-MS (ESI⁺) m/z 472.2. (M+H)⁺.

### (R)-10-methyl-3-(2-(piperazin-1-yl)pyrimidin-5-yl)-9,10,11,12-tetrahydro-8H-[1,4]diazepino[5′,6′:4,5]thieno[3,2-f]quinolin-8-one (Intermediate EO)

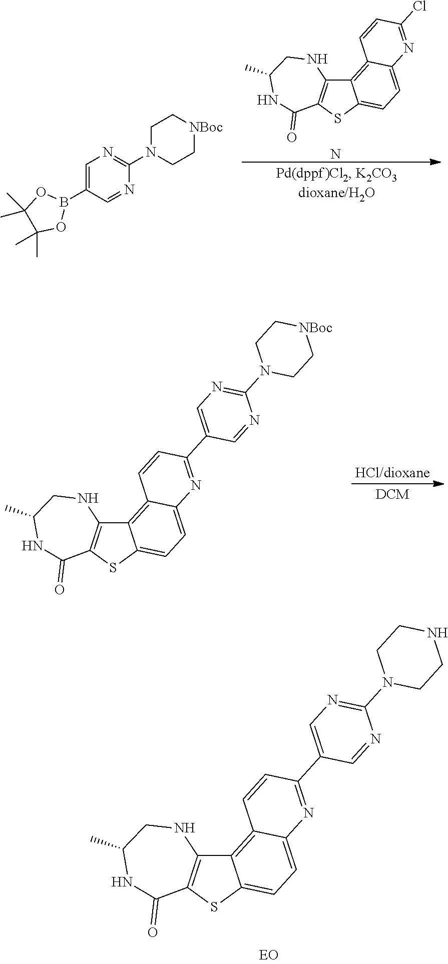

Step 1—(R)-tert-butyl 4-(5-(10-methyl-8-oxo-9,10,11,12-tetrahydro-8H-[1,4]diazepino[5′,6′:4,5]thieno[3,2-f]quinolin-3-yl)pyrimidin-2-yl)piperazine-1-carboxylate. To a solution of tert-butyl 4-(5-(4,4,5,5-tetramethyl-1,3,2-dioxaborolan-2-yl)pyrimidin-2-yl)piperazine-1-carboxylate (737 mg, 1.89 mmol, CAS #940284-98-0) and (R)-3-chloro-10-methyl-9,10,11,12-tetrahydro-8H-[1,4]diazepino[5′,6′:4,5]thieno[3,2-f]quinolin-8-one (500 mg, 1.57 mmol, Intermediate N) in dioxane (12.5 mL) and H₂O (2.50 mL) was added 1,3-bis[2,6-bis(1-propylbutyl)phenyl]-4,5-dichloro-2H-imidazol-1-ium-2-ide 3-chloropyridine dichloropalladium (153 mg, 157 umol) and K₂CO₃ (2 M, 2.36 mL) at 25° C., then the reaction was stirred at 80° C. for 1 hr. On completion, the reaction mixture was filtered and concentrated in vacuo to give the title compound (2 g) as yellow solid. LC-MS (ESI⁺) m/z 545.6. (M+H)⁺.

Step 2—(R)-10-methyl-3-(2-(piperazin-1-yl)pyrimidin-5-yl)-9,10,11,12-tetrahydro-8H-[1,4]diazepino[5′,6′:4,5]thieno[3,2-f]quinolin-8-one. To a solution of (R)-tert-butyl 4-(5-(10-methyl-8-oxo-9,10,11,12-tetrahydro-8H-[1,4]diazepino[5′,6′:4,5]thieno[3,2-f]quinolin-3-yl)pyrimidin-2-yl)piperazine-1-carboxylate (300 mg, 550 umol) in DCM (3 mL) was added HCl/dioxane (4 M, 0.6 mL) at 25° C., then the mixture was stirred at 25° C. for 1 hr. On completion, the reaction mixture was filtered and concentrated in vacuo to give the title compound (250 mg, crude) as red solid. LC-MS (ESI⁺) m/z 446.2. (M+H)⁺.

### 4-(dimethoxymethyl)-1-(4-fluoro-3-(4,4,5,5-tetramethyl-1,3,2-dioxaborolan-2-yl)phenyl)piperidine (Intermediate EP)

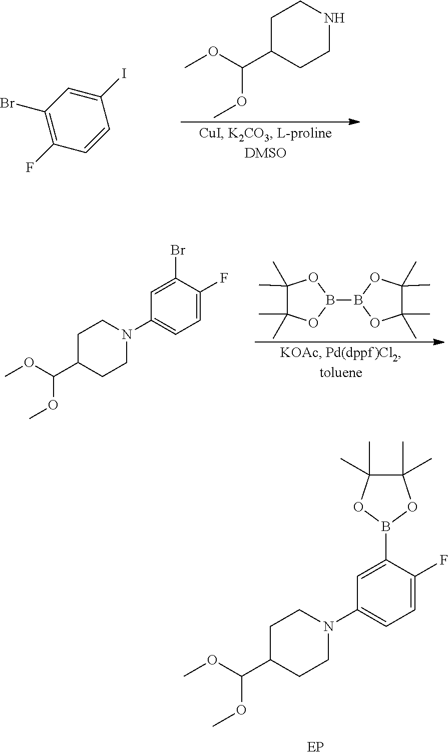

Step 1—1-(3-Bromo-4-fluorophenyl)-4-(dimethoxymethyl)piperidine. To a solution of 2-bromo-1-fluoro-4-iodo-benzene (5 g, 16.6 mmol, CAS #811842-30-5) and 4-(dimethoxymethyl)piperidine (2.65 g, 16.6 mmol, CAS #188646-83-5) in DMSO (100 mL) was added K₂CO₃ (4.59 g, 33.2 mmol) and L-proline (765 mg, 6.65 mmol) and CuI (633 mg, 3.32 mmol). The mixture was degassed and purged with N₂ three times, and then the mixture was stirred at 100° C. for 12 hrs under N₂ atmosphere. On completion, the reaction mixture was diluted with H₂O (100 mL) and extracted with EA (100 mL×3). The combined organic layers were washed with aqueous NaCl (100 mL×3), dried over Na₂SO₄, filtered and concentrated under reduced pressure to give a residue. The residue was purified by column chromatography (SiO2, Petroleum ether/Ethyl acetate=1/0 to 24/1) to give the title compound (2.5 g, 44% yield) as a yellow oil. LC-MS (ESI⁺) m/z 302.0 (M+1)⁺.

Step 2—4-(Dimethoxymethyl)-1-(4-fluoro-3-(4,4,5,5-tetramethyl-1,3,2-dioxaborolan-2-yl)phenyl)piperidine. To a solution of 1-(3-bromo-4-fluoro-phenyl)-4-(dimethoxymethyl)piperidine (1.2 g, 3.61 mmol) and 4,4,5,5-tetramethyl-2-(4,4,5,5-tetramethyl-1,3,2-dioxaborolan-2-yl)-1,3,2-dioxaborolane (3.67 g, 14.4 mmol, 68.0 uL,) in toluene (50 mL) was added KOAc (709 mg, 7.22 mmol) and Pd(PPh₃)₄(626 mg, 541 umol). The mixture was degassed and purged with N₂ three times, and then the mixture was stirred at 100° C. for 12 hr under N₂ atmosphere. On completion, the reaction mixture was diluted and concentrated under reduced pressure to give a residue. The residue was purified by column chromatography (SiO₂, Petroleum ether/Ethyl acetate=1/0 to 10/1) to give the title compound (300 mg, 20% yield) as a red solid. LC-MS (ESI⁺) m/z 380.0 (M+1)⁺.

### (R)-1-(4-fluoro-3-(10-methyl-8-oxo-9,10,11,12-tetrahydro-8H-[1,4]diazepino[5′,6′:4,5]thieno[3,2-f]quinolin-3-yl)phenyl)piperidine-4-carbaldehyde (Intermediate EQ)

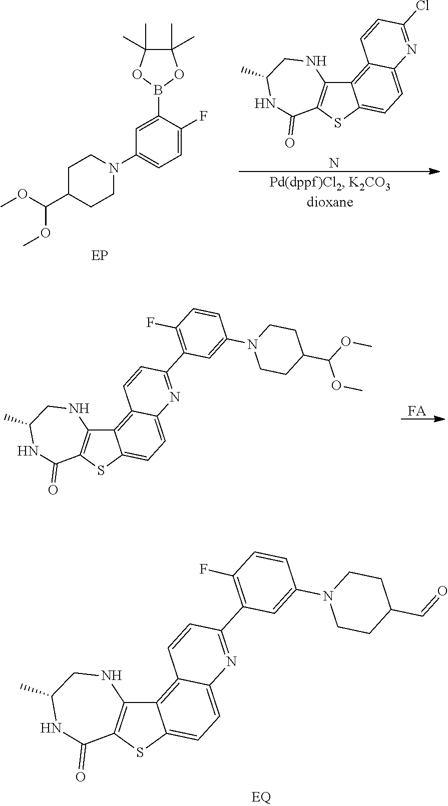

Step 1—(R)-3-(5-(4-(dimethoxymethyl)piperidin-1-yl)-2-fluorophenyl)-10-methyl-9,10,11,12-tetrahydro-8H-[1,4]diazepino[5′,6′:4,5]thieno[3,2-f]quinolin-8-one. To a solution of 4-(dimethoxymethyl)-1-[4-fluoro-3-(4,4,5,5-tetramethyl-1,3,2-dioxaborolan-2-yl)phenyl]piperidine (263 mg, 692 umol, Intermediate EP) and (15R)-5-chloro-15-methyl-11-thia-6,14,17-triazatetracyclo[8.8.0.0^{2,7}.0^{12,18}]octadeca-1,3,5,7,9,12(18)-hexaen-13-one (200 mg, 629 umol, Intermediate N) in dioxane (8 mL) and H₂O (2 mL) was added K₂CO₃ (261 mg, 1.89 mmol) and Pd(dppf)Cl₂ (46.0 mg, 62.9 umol). The mixture was then stirred at 80° C. for 2 hrs. On completion, the reaction mixture was diluted with EA (20 mL) and PE (10 mL), then was filtered and concentrated under reduced pressure to give the title compound (180 mg) as a green solid. LC-MS (ESI⁺) m/z 535.4 (M+1)⁺.

Step 2—(R)-1-(4-fluoro-3-(10-methyl-8-oxo-9,10,11,12-tetrahydro-8H-[1,4]diazepino[5′,6′:4,5]thieno[3,2-f]quinolin-3-yl)phenyl)piperidine-4-carbaldehyde. To a solution of (15R)-5-[5-[4-(dimethoxymethyl)-1-piperidyl]-2-fluoro-phenyl]-15-methyl-1l-thia-6,14,17-triazatetracyclo[8.8.0.0^{2,7}.0^{12,18}]octadeca-1,3,5,7,9,12(18)-hexaen-13-one (180 mg, 337 umol) was dissolved in HCOOH (2 mL). The mixture was stirred at 50° C. for 1 hr. On completion, the reaction mixture was concentrated under reduced pressure to give the title compound (180 mg) as a red solid. LC-MS (ESI⁺) m/z 507.1 (M+1)⁺.

### 8-(1-(2,6-dioxopiperidin-3-yl)-3-methyl-2-oxo-2,3-dihydro-1H-benzo[d]imidazol-4-yl)octanal (Intermediate ER)

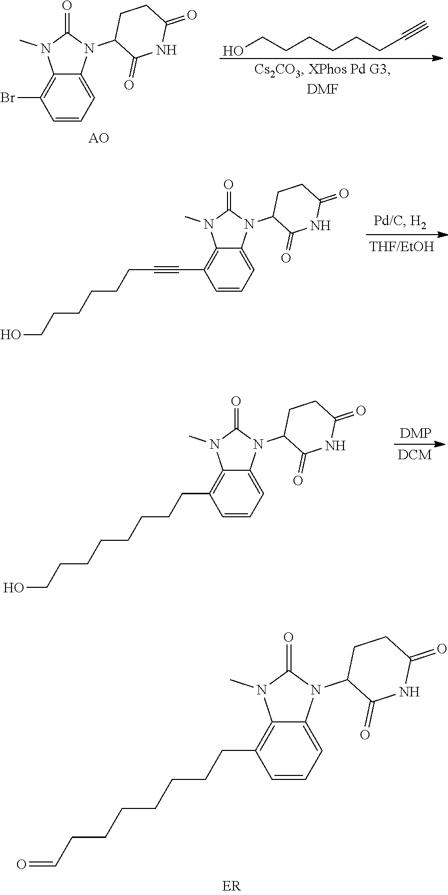

Step 1—3-(4-(8-Hydroxyoct-1-yn-1-yl)-3-methyl-2-oxo-2,3-dihydro-1H-benzo[d]imidazol-1-yl)piperidine-2,6-dione. A mixture of 3-(4-bromo-3-methyl-2-oxo-benzimidazol-1-yl)piperidine-2,6-dione (2 g, 5.91 mmol, Intermediate AO), oct-7-yn-1-ol (970 mg, 7.69 mmol), Cs₂CO₃ (9.64 g, 29.6 mmol), and XPhos Pd G3 (501 mg, 591 umol) in DMF (50 mL) was degassed and purged with N₂ three times, and then the mixture was stirred at 60° C. for 4 hours under N₂ atmosphere. On completion, the reaction mixture was filtered to give the residue, and then diluted with H₂O (50 mL) and extracted with EA (50 mL×5). The combined organic layers were washed with brine (60 mL×3), dried over Na₂SO₄, filtered and concentrated under reduced pressure to give a residue. The residue was purified by column chromatography (SiO₂, Petroleum ether/Ethyl acetate=1/1 to 0/1) to give the title compound (1.1 g, 46% yield) as a yellow solid. LC-MS (ESI⁺) m/z 384.1 (M+H)⁺.

Step 2—3-(4-(8-Hydroxyoctyl)-3-methyl-2-oxo-2,3-dihydro-1H-benzo[d]imidazol-1-yl)piperidine-2,6-dione. To a solution of 3-[4-(8-hydroxyoct-1-ynyl)-3-methyl-2-oxo-benzimidazol-1-yl]piperidine-2,6-dione (300 mg, 782 umol) in THF (5 mL) and EtOH (5 mL) was added Pd/C (10 wt %, 300 mg) under N₂ atmosphere. The suspension was degassed and purged with H₂ 3 times. The mixture was stirred under H₂ (15 Psi or atm.) at 25° C. for 4 hrs. On completion, the reaction mixture was filtered carefully through celite. Then the filtrate was concentrated under reduced pressure to give the title compound as a white solid (292 mg). LC-MS (ESI⁺) m/z 388.1 (M+H)⁺.

Step 3—8-(1-(2,6-Dioxopiperidin-3-yl)-3-methyl-2-oxo-2,3-dihydro-1H-benzo[d]imidazol-4-yl)octanal. To a solution of 3-[4-(8-hydroxyoctyl)-3-methyl-2-oxo-benzimidazol-1-yl]piperidine-2,6-dione (50 mg, 129 umol) in DCM (4 mL) was added DMP (71.1 mg, 168 umol) at 0° C. Then the mixture was stirred at 25° C. for 3 hrs. On completion, the reaction mixture was quenched with Na₂S203 (10 mL), and then diluted with H₂O (10 mL) and extracted with DCM (20 mL×3). The combined organic layers were washed with brine (30 mL×3), dried over Na₂SO₄, filtered and concentrated under reduced pressure to give the title compound (36 mg) as a yellow solid. LC-MS (ESI⁺) m/z 386.1 (M+H)⁺.

### (R)-3-(2-fluoro-5-hydroxyphenyl)-10-methyl-9,10,11,12-tetrahydro-8H-[1,4]diazepino[5′,6′:4,5]thieno[3,2-f]quinolin-8-one (Intermediate ES)

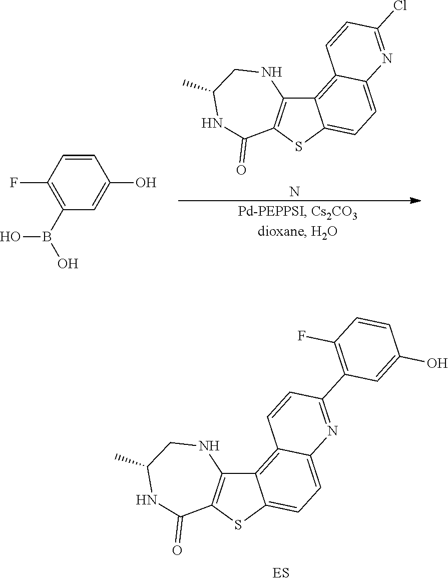

A mixture of (R)-3-chloro-10-methyl-9,10,11,12-tetrahydro-8H-[1,4]diazepino[5′,6′:4,5]thieno[3,2-f]quinolin-8-one (1 g, 3.15 mmol, Intermediate N), (2-fluoro-5-hydroxyphenyl)boronic acid (982 mg, 6.3 mmol, CAS #1150114-52-5), 1,3-bis[2,6-bis(1-propylbutyl)phenyl]-4,5-dichloro-2H-imidazol-1-ium-2-ide 3-chloropyridine dichloropalladium (306 mg, 315 umol), and Cs₂CO₃ (3.08 g, 9.45 mmol) in dioxane (10 mL) and H₂O (3 mL) was degassed and purged with N₂ three times. Then the mixture was stirred at 80° C. for 2 hrs under N₂ atmosphere. On completion, the reaction mixture was filtered and concentrated under reduced pressure to give the title compound (1.2 g) as a black solid. LC-MS (ESI⁺) m/z 394.1 (M+H)⁺.

### (R)-4-fluoro-3-(10-methyl-8-oxo-9,10,11,12-tetrahydro-8H-[1,4]diazepino[5′,6′:4,5]thieno[3,2-f]quinolin-3-yl)phenyl trifluoromethanesulfonate (Intermediate ET)

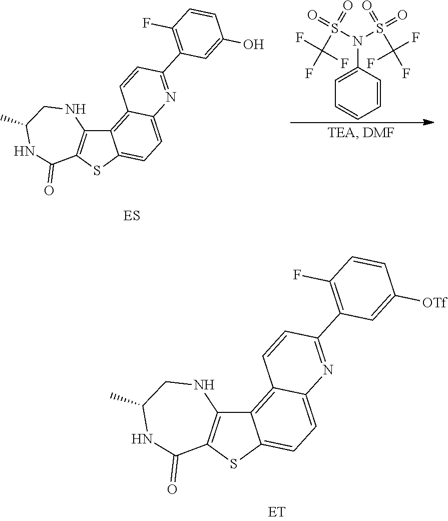 [SMILES: C[C@@H]1CNc2c(sc3ccc4nc(-c5cc(O)ccc5F)ccc4c23)C(=O)N1 ; O=S(=O)(N(c1ccccc1)S(=O)(=O)C(F)(F)F)C(F)(F)F ; C[C@@H]1CNc2c(sc3ccc4nc(-c5cc(OS(=O)(=O)C(F)(F)F)ccc5F)ccc4c23)C(=O)N1]

To a solution of (R)-3-(2-fluoro-5-hydroxyphenyl)-10-methyl-9,10,11,12-tetrahydro-8H-[1,4]diazepino[5′,6′:4,5]thieno[3,2-f]quinolin-8-one (1.2 g, 3.05 mmol, Intermediate ES) and 1,1,1-trifluoro-N-phenyl-N-((trifluoromethyl)sulfonyl)methanesulfonamide (2.18 g, 6.1 mmol, CAS #37595-74-7) in DMF (20 mL) was added TEA (617 mg, 6.1 mmol). The mixture was stirred at 60° C. for 12 hrs. On completion, the reaction mixture was quenched with H₂O (100 mL) at 25° C., and then extracted with EA (100 mL×3). The combined organic layers dried over Na₂SO₄, filtered and concentrated under reduced pressure to give a residue. The residue was purified by column chromatography (SiO₂, Petroleum ether/Ethyl acetate=5/1 to 0/1) to give the title compound (1.1 g, 48% yield) as a yellow solid. LC-MS (ESI+) m/z 525.9 (M+H)⁺.

### (R)-3-(2-fluoro-5-(piperazin-1-yl)phenyl)-10-methyl-9,10,11,12-tetrahydro-8H-[1,4]diazepino[5′,6′:4,5]thieno[3,2-f]quinolin-8-one (Intermediate EU)

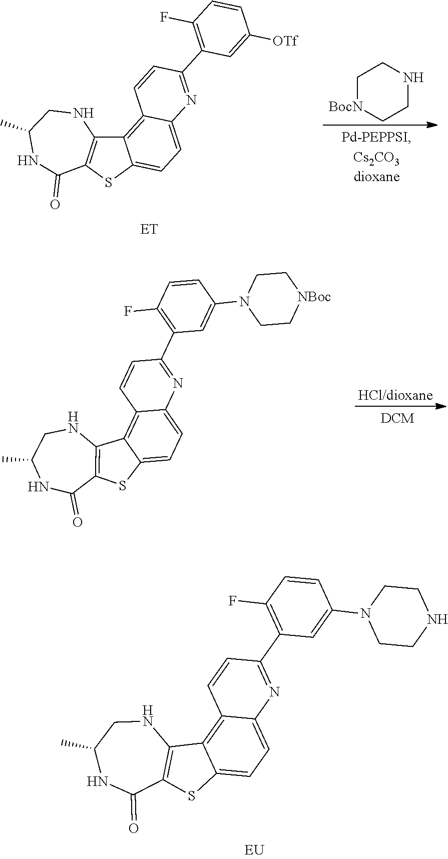 [SMILES: C[C@@H]1CNc2c(sc3ccc4nc(-c5cc(OS(=O)(=O)C(F)(F)F)ccc5F)ccc4c23)C(=O)N1 ; CC(C)(C)OC(=O)N1CCNCC1 ; C[C@@H]1CNc2c(sc3ccc4nc(-c5cc(N6CCN(C(=O)OC(C)(C)C)CC6)ccc5F)ccc4c23)C(=O)N1 ; C[C@@H]1CNc2c(sc3ccc4nc(-c5cc(N6CCNCC6)ccc5F)ccc4c23)C(=O)N1]

Step 1—(R)-tert-butyl 4-(4-fluoro-3-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. (R)-4-fluoro-3-(10-methyl-8-oxo-9,10,11,12-tetrahydro-8H-[1,4]diazepino[5′,6′:4,5]thieno[3,2-f]quinolin-3-yl)phenyl trifluoromethanesulfonate (400 mg, 761 umol, Intermediate ET), tert-butyl piperazine-1-carboxylate (283 mg, 1.52 mmol, CAS #143238-38-4) and 1,3-bis[2,6-bis(1-propylbutyl)phenyl]-4,5-dichloro-2H-imidazol-1-ium-2-ide 3-chloropyridine dichloropalladium (74 mg, 76.1 umol), and Cs₂CO₃ (744 mg, 2.28 mmol) were taken up into a microwave tube in dioxane (10 mL). The sealed tube was heated at 130° C. for 2 hrs under microwave. On completion, the reaction mixture was concentrated under reduced pressure to remove solvent. The residue was purified by column chromatography (SiO₂, Petroleum ether/Ethyl acetate=5/1 to 0/1) to give the title compound (330 mg, 77% yield) as a yellow solid. LC-MS (ESI+) m/z 562.4 (M+H)⁺.

Step 2—(R)-3-(2-fluoro-5-(piperazin-1-yl)phenyl)-10-methyl-9,10,11,12-tetrahydro-8H-[1,4]diazepino[5′,6′:4,5]thieno[3,2-f]quinolin-8-one. To a solution of (R)-tert-butyl 4-(4-fluoro-3-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (300 mg, 534 umol) in DCM (5 mL) was added HCl/dioxane (4 M, 1 mL). The mixture was stirred at 25° C. for 2 hrs. On completion, the reaction mixture was concentrated under reduced pressure to give the title compound (300 mg) as a yellow solid. LC-MS (ESI+) m/z 462.2 (M+H)⁺.

### (R)-2-(2-chloropyridin-4-yl)-6-methyl-6,7-dihydro-1H-pyrrolo[3,2-c]pyridin-4(5H)-one (Intermediate EV)

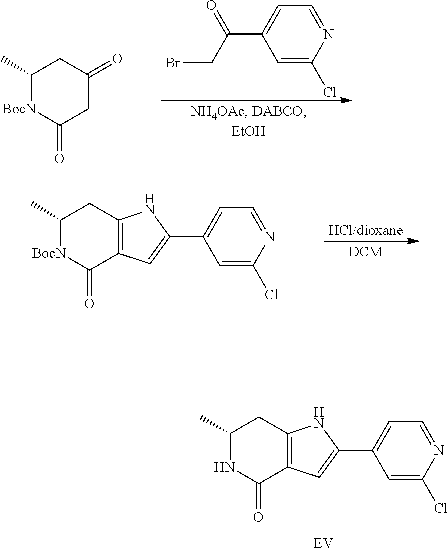 [SMILES: C[C@@H]1CC(=O)CC(=O)N1C(=O)OC(C)(C)C ; O=C(CBr)c1ccnc(Cl)c1 ; C[C@@H]1Cc2[nH]c(-c3ccnc(Cl)c3)cc2C(=O)N1C(=O)OC(C)(C)C ; C[C@@H]1Cc2[nH]c(-c3ccnc(Cl)c3)cc2C(=O)N1]

Step 1—(R)-tert-butyl 2-(2-chloropyridin-4-yl)-6-methyl-4-oxo-6,7-dihydro-1H-pyrrolo[3,2-c]pyridine-5(4H)-carboxylate. A mixture of (R)-tert-butyl 2-methyl-4,6-dioxopiperidine-1-carboxylate (4.00 g, 17.6 mmol, synthesized via Step 1 of Intermediate AG), 2-bromo-1-(2-chloropyridin-4-yl)ethanone (4.54 g, 19.4 mmol, synthesis of Step 1 of Intermediate AC), NH₄OAc (1.36 g, 17.6 mmol) and DABCO (98.7 mg, 880 umol, 96.8 uL) in EtOH (60 mL) was degassed and purged with N₂ three times. The mixture was stirred at 25° C. for 12 hrs under N₂ atmosphere. On completion, the mixture was filtered and concentrated in vacuo to get the crude residue. Then water (80.0 mL) was added and the mixture was extracted with dichloromethane (40×3 mL). The extracts were dried over anhydrous sodium sulfate, filtered and concentrated in vacuo to get the crude residue. The residue was purified by column chromatography (SiO₂, Petroleum ether/Ethyl acetate=5/1 to 2/1) to give the title compound (2 g, 29% yield) as yellow solid. ¹H NMR (400 MHz, DMSO-d6) δ ppm 1.22 (br d, J=6.8 Hz, 3H) 1.45-1.51 (m, 9H) 2.71 (br d, J=16.8 Hz, 1H) 4.50-4.74 (m, 1H) 5.66-5.82 (m, 1H) 7.26 (s, 1H) 7.61-7.81 (m, 2H) 8.24-8.40 (m, 1H) 12.21 (br s, 1H). LC-MS (ESI⁺) m/z 380.1. (M+H)⁺.

Step 2—(R)-2-(2-chloropyridin-4-yl)-6-methyl-6,7-dihydro-1H-pyrrolo[3,2-c]pyridin-4(5H)-one. To a solution of (R)-tert-butyl 2-(2-chloropyridin-4-yl)-6-methyl-4-oxo-6,7-dihydro-1H-pyrrolo[3,2-c]pyridine-5(4H)-carboxylate (1.3 g, 3.6 mmol) in DCM (12 mL) was added HCl/dioxane (4 M, 2.5 mL) at 25° C., then the mixture was stirred at 25° C. for 2 hrs. On completion, the reaction mixture was filtered and concentrated in vacuo to give the title compound (170 mg) as a yellow solid. LC-MS (ESI⁺) m/z 262.1. (M+H)⁺.

### (R)-6-methyl-2-(2-(4-(piperazin-1-yl)phenyl)pyridin-4-yl)-6,7-dihydro-1H-pyrrolo[3,2-c]pyridin-4(5H)-one (Intermediate EW)

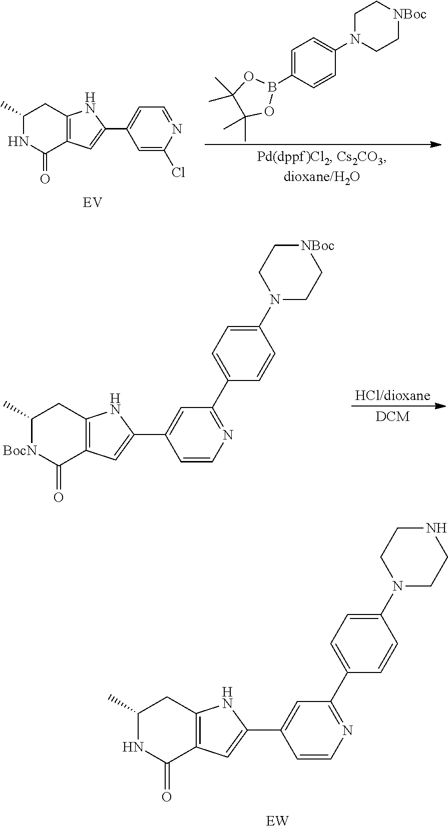

Step 1—(R)-tert-butyl 4-(4-(4-(6-methyl-4-oxo-4,5,6,7-tetrahydro-1H-pyrrolo[3,2-c]pyridin-2-yl)pyridin-2-yl)phenyl)piperazine-1-carboxylate. To a solution of (R)-2-(2-chloropyridin-4-yl)-6-methyl-6,7-dihydro-1H-pyrrolo[3,2-c]pyridin-4(5H)-one (130 mg, 496.7 umol, Intermediate EV) and tert-butyl 4-(4-(4,4,5,5-tetramethyl-1,3,2-dioxaborolan-2-yl)phenyl)piperazine-1-carboxylate (289 mg, 745 umol, CAS #470478-90-1) in H₂O (1.5 mL) and dioxane (7 mL) was added Cs₂CO₃ (485 mg, 1.5 mmol) and Pd-PEPPSI-IHeptCl (48.3 mg, 49.7 umol) at 25° C. The mixture was stirred at 80° C. for 2 hrs. On completion, the reaction mixture was quenched with H₂O (10 mL) at 25° C., and then diluted with H₂O (20 mL) and extracted with DCM (20 mL×4). The organic layer was dried over Na₂SO₄, filtered and concentrated under reduced pressure to give a residue. The residue was purified by column chromatography (SiO₂, Petroleum ether/Ethyl acetate=5/1 to DCM/Ethyl acetate=10/1) to give the title compound (220 mg, 65% yield) as yellow solid. LC-MS (ESI⁺) m/z 488.3. (M+H)⁺.

Step 2—(R)-6-methyl-2-(2-(4-(piperazin-1-yl)phenyl)pyridin-4-yl)-6,7-dihydro-1H-pyrrolo[3,2-c]pyridin-4(5H)-one. To a solution of (R)-tert-butyl 4-(4-(4-(6-methyl-4-oxo-4,5,6,7-tetrahydro-1H-pyrrolo[3,2-c]pyridin-2-yl)pyridin-2-yl)phenyl)piperazine-1-carboxylate (100 mg, 205.1 umol) in DCM (1 mL) was added HCl/dioxane (4 M, 0.2 mL) at 25° C., then the mixture was stirred at 25° C. for 2 hrs. On completion, the reaction mixture was filtered and concentrated in vacuo to give the title compound (1.25 g) as yellow solid. LC-MS (ESI⁺) m/z 388.1. (M+H)⁺.

### (1-(3-chloro-4-(4,4,5,5-tetramethyl-1,3,2-dioxaborolan-2-yl)phenyl)piperidin-4-yl)methanol (Intermediate EX)

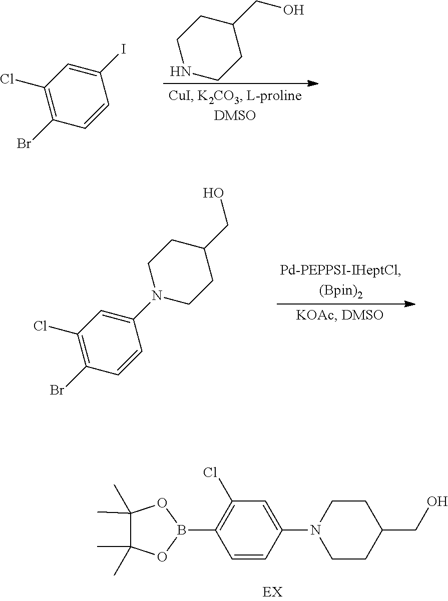 [SMILES: Clc1cc(I)ccc1Br ; OCC1CCNCC1 ; OCC1CCN(c2ccc(Br)c(Cl)c2)CC1 ; CC1(C)OB(c2ccc(N3CCC(CO)CC3)cc2Cl)OC1(C)C]

Step 1—(1-(4-Bromo-3-chlorophenyl)piperidin-4-yl)methanol. To a solution of 1-bromo-2-chloro-4-iodobenzene (5 g, 15.8 mmol) and piperidin-4-ylmethanol (2.72 g, 23.6 mmol) in DMSO (50 mL) was added L-proline (726 mg, 6.30 mmol), CuI (600 mg, 3.15 mmol) and K₂CO₃ (4.36 g, 31.5 mmol) at 25° C., then the reaction was stirred at 100° C. for 12 hrs. On completion, the reaction mixture was quenched with water (200 mL) and extracted by ethyl acetate (3×100 mL). The extracts were washed with brine (200 mL) and dried over anhydrous sodium sulfate, filtered and concentrated in vacuo to get the crude residue. The residue was purified by column chromatography (SiO₂, Petroleum ether/Ethyl acetate=10/1 to 3/1) to give the title compound (3.62 g, 75% yield) as a yellow solid. LC-MS (ESI⁺) m/z 305.9. (M+H)⁺.

Step 2—(1-(3-Chloro-4-(4,4,5,5-tetramethyl-1,3,2-dioxaborolan-2-yl)phenyl)piperidin-4-yl)methanol. (1-(4-bromo-3-chlorophenyl)piperidin-4-yl)methanol (1 g, 3.28 mmol), 4,4,4′,4′,5,5,5′,5′-octamethyl-2,2′-bi(1,3,2-dioxaborolane) (2.08 g, 8.21 mmol), KOAc (967 mg, 9.85 mmol), and Pd-PEPPSI-IHeptCl (319 mg, 328 umol, CAS #1814936-54-3) were taken up into a microwave tube in DMSO (15 mL). The sealed tube was heated at 120° C. for 3 hrs under microwave. On completion, the reaction mixture was quenched with water (30 mL) and extracted with ethyl acetate (3×50 mL). The extracts were washed with brine (100 mL) and dried over anhydrous sodium sulfate, filtered and concentrated in vacuo to get the crude residue. The residue was purified by prep-HPLC (0.1% FA) to give the title compound (420 mg, 13% yield) as a white solid. LC-MS (ESI⁺) m/z 351.9. (M+H)⁺.

### (R)-1-(3-chloro-4-(10-methyl-8-oxo-9,10,11,12-tetrahydro-8H-[1,4]diazepino[5′,6′:4,5]thieno[3,2-f]quinolin-3-yl)phenyl)piperidine-4-carbaldehyde (Intermediate EY)

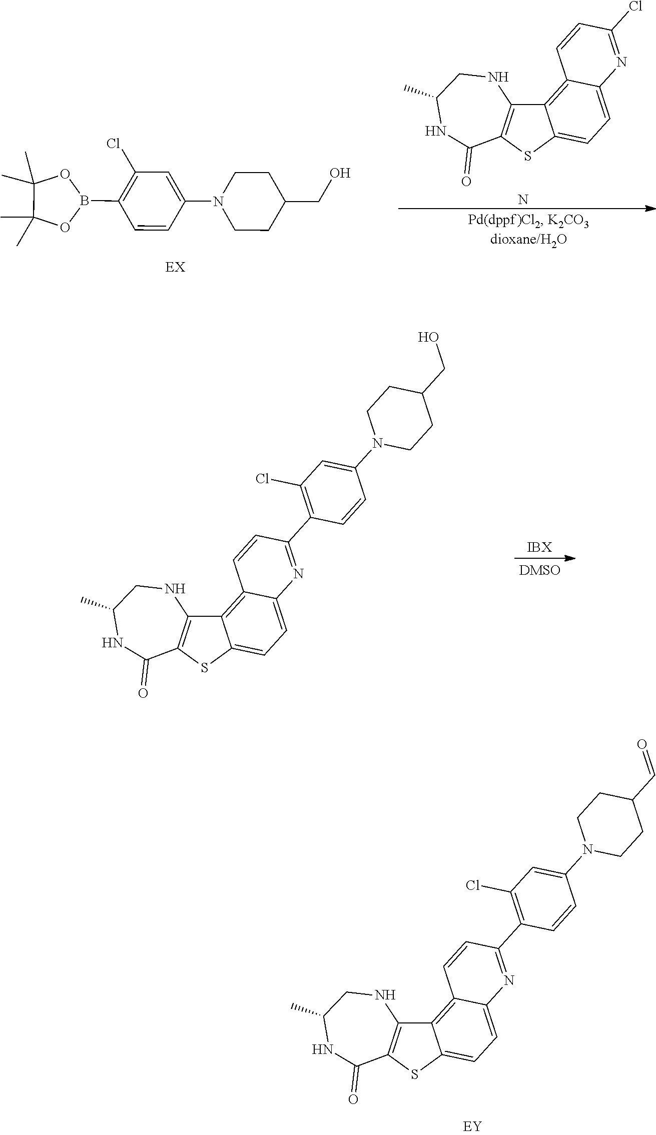

Step 1—(R)-3-(2-chloro-4-(4-(hydroxymethyl)piperidin-1-yl)phenyl)-10-methyl-9,10,11,12-tetrahydro-8H-[1,4]diazepino[5′,6′:4,5]thieno[3,2-f]quinolin-8-one. A mixture of (R)-3-chloro-10-methyl-9,10,11,12-tetrahydro-8H-[1,4]diazepino[5′,6′:4,5]thieno[3,2-f]quinolin-8-one (175 mg, 551 umol, Intermediate N), (1-(3-chloro-4-(4,4,5,5-tetramethyl-1,3,2-dioxaborolan-2-yl)phenyl)piperidin-4-yl)methanol (273 mg, 661 umol, Intermediate EX), Pd(dppf)Cl₂ (40.3 mg, 55.1 umol), and K₂CO₃ (228 mg, 1.65 mmol) in dioxane (4 mL) and H₂O (1 mL) was degassed and purged with N₂ three times. Then the mixture was stirred at 80° C. for 2 hrs under N₂ atmosphere. On completion, the reaction mixture was filtered and concentrated under reduced pressure to give a residue. The residue was purified by prep-HPLC (0.1% FA) to give the title compound (100 mg, 32% yield, FA) as a white solid. LC-MS (ESI⁺) m/z 507.5. (M+H)⁺.

Step 2—(R)-1-(3-chloro-4-(10-methyl-8-oxo-9,10,11,12-tetrahydro-8H-[1,4]diazepino[5′,6′:4,5]thieno[3,2-f]quinolin-3-yl)phenyl)piperidine-4-carbaldehyde. To a solution of (R)-3-(2-chloro-4-(4-(hydroxymethyl)piperidin-1-yl)phenyl)-10-methyl-9,10,11,12-tetrahydro-8H-[1,4]diazepino[5′,6′:4,5]thieno[3,2-f]quinolin-8-one (40 mg, 78.9 umol) in DMSO (1 mL) was added IBX (44.2 mg, 158 umol). The mixture was stirred at 35° C. for 24 hrs. On completion, the reaction mixture was quenched with water (5 mL) and extracted with ethyl acetate (3×10 mL). The extracts were washed with brine (10 mL) and dried over anhydrous sodium sulfate, filtered and concentrated in vacuo to give the title compound as yellow solid (60 mg). LC-MS (ESI⁺) m/z 505.4. (M+H)⁺.

### 6-(1-(2,6-dioxopiperidin-3-yl)-3-methyl-2-oxo-2,3-dihydro-1H-benzo[d]imidazol-4-yl)hexanal (Intermediate EZ)

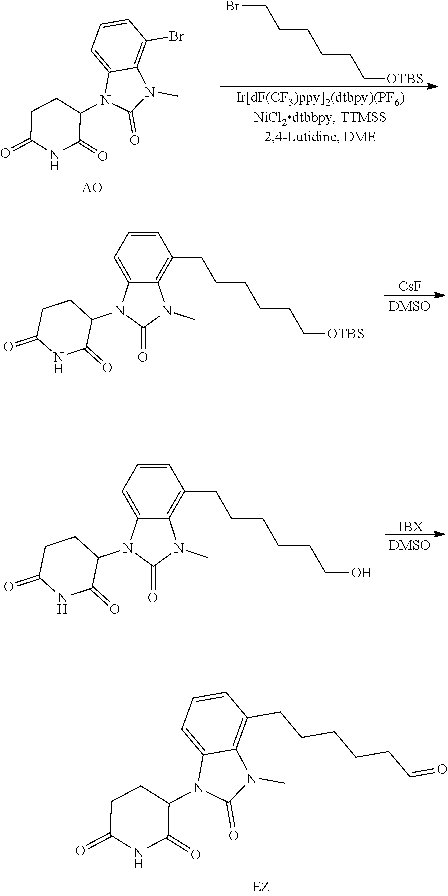

Step 1—3-(4-(6-((Tert-butyldimethylsilyl)oxy)hexyl)-3-methyl-2-oxo-2,3-dihydro-1H-benzo[d]imidazol-1-yl)piperidine-2,6-dione. To an 15 mL vial equipped with a stir bar was added 3-(4-bromo-3-methyl-2-oxo-benzimidazol-1-yl)piperidine-2,6-dione (3 g, 8.87 mmol, Intermediate AO), 6-bromohexoxy-tert-butyl-dimethyl-silane (3.41 g, 11.5 mmol, CAS #129368-70-3), Ir[dF(CF₃)ppy]₂(dtbpy)(PF₆) (199 mg, 177 umol), NiCl₂.dtbbpy (106 mg, 266 umol), TTMSS (2.21 g, 8.87 mmol), and 2,6-lutidine (1.90 g, 17.7 mmol) in DME (140 mL). The vial was sealed and placed under nitrogen then the reaction was stirred and irradiated with a 10 W blue LED lamp (3 cm away), with cooling water to keep the reaction temperature at 25° C. for 14 hrs. On completion, the reaction mixture was filtered to give a residue. The residue was purified by column chromatography (SiO₂, Petroleum ether/Ethyl acetate=1/0 to 0/1) to give the title compound (2.32 g, 42% yield) as yellow solid. LC-MS (ESI+) m/z 474.5. (M+H)⁺.

Step 2—3-(4-(6-Hydroxyhexyl)-3-methyl-2-oxo-2,3-dihydro-1H-benzo[d]imidazol-1-yl)piperidine-2,6-dione. To a solution of 3-(4-(6-((tert-butyldimethylsilyl)oxy)hexyl)-3-methyl-2-oxo-2,3-dihydro-1H-benzo[d]imidazol-1-yl)piperidine-2,6-dione (0.5 g, 1.06 mmol) in DMSO (10 mL) was added CsF (1.60 g, 10.6 mmol) at 25° C., then the reaction was stirred at 25° C. for 2 hrs. On completion, the reaction mixture was filtered to get the crude residue. The crude product was purified by reversed-phase HPLC (0.1% FA condition) to give the title compound (0.34 g, 90% yield) as white solid. LC-MS (ESI+) m/z 360.0. (M+H)⁺.

Step 3—6-(1-(2,6-Dioxopiperidin-3-yl)-3-methyl-2-oxo-2,3-dihydro-1H-benzo[d]imidazol-4-yl)hexanal. To a solution of 3-(4-(6-hydroxyhexyl)-3-methyl-2-oxo-2,3-dihydro-1H-benzo[d]imidazol-1-yl)piperidine-2,6-dione (200 mg, 556 umol) in DMSO (2 mL) was added IBX (312 mg, 1.11 mmol) at 25° C., the reaction was stirred at 25° C. for 3 hrs. On completion, the reaction mixture was concentrated in vacuo to get the crude residue. The crude product was purified by reversed-phase HPLC (0.1% FA condition) to give the title compound (120 mg, 60% yield) as white solid. LC-MS (ESI+) m/z 358.0. (M+H)⁺.

### Tert-butyl 7-(4-(4,4,5,5-tetramethyl-1,3,2-dioxaborolan-2-yl)phenyl)-2,7-diazaspiro[3.5]nonane-2-carboxylate (Intermediate FA)

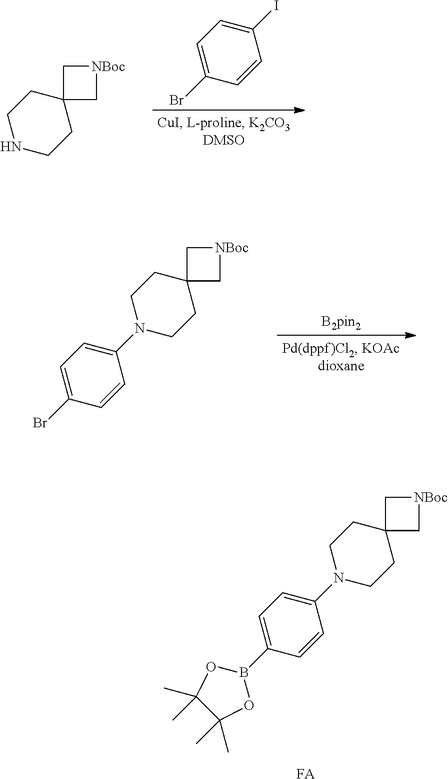 [SMILES: CC(C)(C)OC(=O)N1CC2(CCNCC2)C1 ; Brc1ccc(I)cc1 ; CC(C)(C)OC(=O)N1CC2(CCN(c3ccc(Br)cc3)CC2)C1 ; CC(C)(C)OC(=O)N1CC2(CCN(c3ccc(B4OC(C)(C)C(C)(C)O4)cc3)CC2)C1 ; "FA"]

Step 1—Tert-butyl 7-(4-bromophenyl)-2,7-diazaspiro[3.5]nonane-2-carboxylate. To a solution of 1-bromo-4-iodobenzene (7.50 g, 26.5 mmol, CAS #589-87-7) in DMSO (50 mL) was added tert-butyl 2,7-diazaspiro[3.5]nonane-2-carboxylate (6 g, 26.5 mmol, CAS #236406-55-6), CuI (1.01 g, 5.30 mmol), L-proline (1.22 g, 10.6 mmol) and K₂CO₃ (7.33 g, 53.0 mmol), then purged with N₂ three times. Then the mixture was stirred at 80° C. for 2 hrs under N₂ atmosphere. On completion, the mixture was quenched with H₂O (200 mL), then extracted with EtOAc (100 mL×3). The organic later was washed with brine (100 mL×3), then concentrated under reduced pressure to give the title compound (10 g) as a brown oil. LC-MS (ESI⁺) m/z 383.1 (M+H)⁺.

Step 2—Tert-butyl 7-(4-(4,4,5,5-tetramethyl-1,3,2-dioxaborolan-2-yl)phenyl)-2,7-diazaspiro[3.5]nonane-2-carboxylate. To a solution of tert-butyl 7-(4-bromophenyl)-2,7-diazaspiro[3.5]nonane-2-carboxylate (1.6 g, 3.36 mmol) in dioxane (20 mL) was added 4,4,5,5-tetramethyl-2-(4,4,5,5-tetramethyl-1,3,2-dioxaborolan-2-yl)-1,3,2-dioxaborolane (1.70 g, 6.71 mmol), Pd(dppf)Cl₂ (246 mg, 336 umol) and KOAc (988 mg, 10.1 mmol), the reaction was degassed and purged with N₂ three times. Then the mixture was stirred at 80° C. for 4 hrs under N₂ atmosphere. On completion, the mixture was concentrated under reduced pressure to give a residue. The residue was purified by column chromatography (SiO₂, Petroleum ether/Ethyl acetate=50/1 to 5/1) to give the title compound (1.2 g, 76% yield) as a white solid. LC-MS (ESI⁺) m/z 429.3 (M+H)⁺.

### (R)-3-(4-(2,7-diazaspiro[3.5]nonan-7-yl)phenyl)-10-methyl-9,10,11,12-tetrahydro-8H-[1,4]diazepino[5′,6′:4,5]thieno[3,2-f]quinolin-8-one (Intermediate FB)

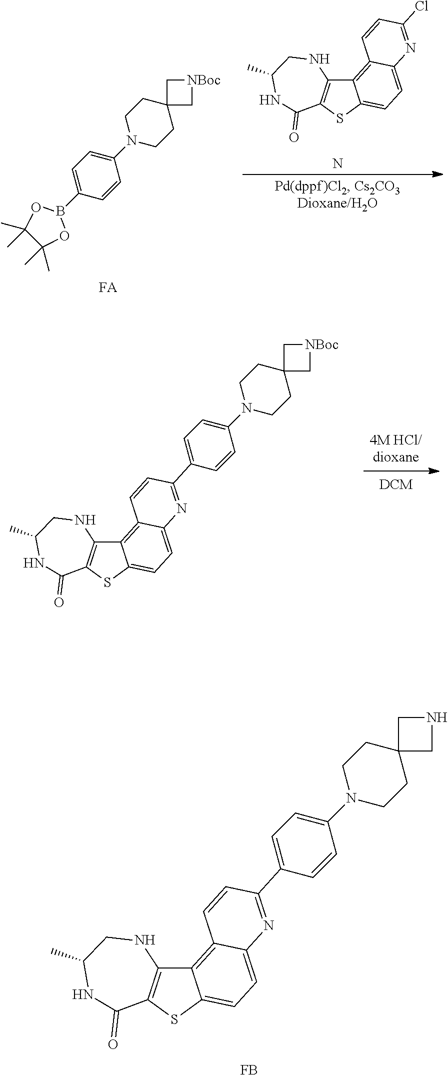

Step 1—(R)-tert-butyl 7-(4-(10-methyl-8-oxo-9,10,11,12-tetrahydro-8H-[1,4]diazepino[5′,6′:4,5]thieno[3,2-f]quinolin-3-yl)phenyl)-2,7-diazaspiro[3.5]nonane-2-carboxylate. To a solution of tert-butyl 7-(4-(4,4,5,5-tetramethyl-1,3,2-dioxaborolan-2-yl)phenyl)-2,7-diazaspiro[3.5]nonane-2-carboxylate (816 mg, 1.90 mmol, Intermediate FA) in dioxane (15 mL) and H₂O (3 mL) was added (R)-3-chloro-10-methyl-9,10,11,12-tetrahydro-8H-[1,4]diazepino[5′,6′:4,5]thieno[3,2-f]quinolin-8-one (550 mg, 1.73 mmol, Intermediate N), Pd(dppf)Cl₂ (127 mg, 173 umol), and K₂CO₃ (598 mg, 4.33 mmol), then the mixture was purged with N₂ three times. Then the mixture was stirred at 80° C. for 2 hrs under N₂ atmosphere. On completion, the mixture was concentrated under reduced pressure to give a residue. The residue was purified by column chromatography (SiO₂, Petroleum ether/Ethyl acetate=5/1 to 1/1, then DCM:MeOH=15:1) to give the title compound (820 mg, 69% yield) as a yellow solid. LC-MS (ESI⁺) m/z 584.3 (M+H)⁺.

Step 2—(R)-3-(4-(2,7-diazaspiro[3.5]nonan-7-yl)phenyl)-10-methyl-9,10,11,12-tetrahydro-8H-[1,4]diazepino[5′,6′:4,5]thieno[3,2-f]quinolin-8-one. To a solution of (R)-tert-butyl 7-(4-(10-methyl-8-oxo-9,10,11,12-tetrahydro-8H-[1,4]diazepino[5′,6′:4,5]thieno[3,2-f]quinolin-3-yl)phenyl)-2,7-diazaspiro[3.5]nonane-2-carboxylate (200 mg, 343 umol) in DCM (15 mL) was added HCl/dioxane (4 M, 1 mL). The mixture was stirred at 20° C. for 0.5 hr. On completion, the mixture was filtered to give the title compound (200 mg) as a red solid. LC-MS (ESI⁺) m/z 484.3 (M+H)⁺.

### Tert-butyl (R)-7-(3-(10-methyl-8-oxo-9,10,11,12-tetrahydro-8H-[1,4]diazepino[5′,6′:4,5]thieno[3,2-f]quinolin-3-yl)phenyl)-2,7-diazaspiro[3.5]nonane-2-carboxylate (Intermediate FC)

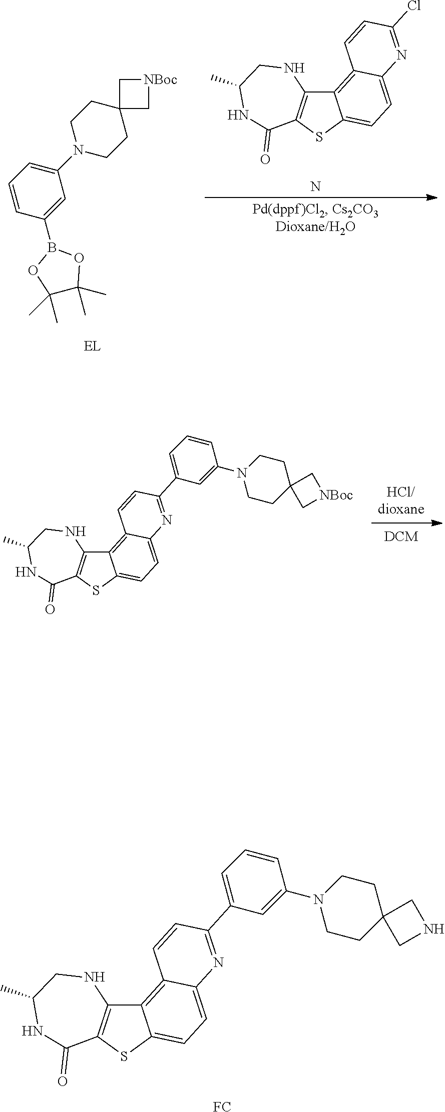

Step 1—(R)-tert-butyl 7-(3-(10-methyl-8-oxo-9,10,11,12-tetrahydro-8H-[1,4]diazepino[5′,6′:4,5]thieno[3,2-f]quinolin-3-yl)phenyl)-2,7-diazaspiro[3.5]nonane-2-carboxylate. To a solution of tert-butyl 7-(3-(4,4,5,5-tetramethyl-1,3,2-dioxaborolan-2-yl)phenyl)-2,7-diazaspiro[3.5]nonane-2-carboxylate (500 mg, 1.17 mmol, Intermediate EL) in dioxane (15 mL) and H₂O (3 mL) was added (R)-3-chloro-10-methyl-9,10,11,12-tetrahydro-8H-[1,4]diazepino[5′,6′:4,5]thieno[3,2-f]quinolin-8-one (309 mg, 973 umol, Intermediate N), Pd(dppf)Cl₂ (71.2 mg, 97.3 umol), and Cs₂CO₃ (951 mg, 2.92 mmol), then the mixture was purged with N₂ three times. Then the mixture was stirred at 80° C. for 2 hrs under N₂ atmosphere. On completion, the mixture was concentrated under reduced pressure to give a residue. The residue was purified by column chromatography (SiO₂, Petroleum ether/Ethyl acetate=10/1 to 1/1) to give the title compound (400 mg) as a white solid. LC-MS (ESI⁺) m/z 584.5 (M+H)⁺.

Step 2—(R)-3-(3-(2,7-diazaspiro[3.5]nonan-7-yl)phenyl)-10-methyl-9,10,11,12-tetrahydro-8H-[1,4]diazepino[5′,6′:4,5]thieno[3,2-f]quinolin-8-one. To a solution of (R)-tert-butyl 7-(4-(10-methyl-8-oxo-9,10,11,12-tetrahydro-8H-[1,4]diazepino[5′,6′:4,5]thieno[3,2-f]quinolin-3-yl)phenyl)-2,7-diazaspiro[3.5]nonane-2-carboxylate (100 mg, 171 umol) in DCM (15 mL) was added HCl/dioxane (4 M, 1 mL), then the mixture was stirred at 25° C. for 3 hr. On completion, the mixture was concentrated under reduced pressure to give the title compound (80 mg, 165 umol) as a red solid. LC-MS (ESI⁺) m/z 484.3 (M+H)⁺.

### 3-fluoro-4-(4-(4-oxo-4,5,6,7-tetrahydro-1H-pyrrolo[3,2-c]pyridin-2-yl)pyridin-2-yl)phenyl trifluoromethanesulfonate (Intermediate FD)

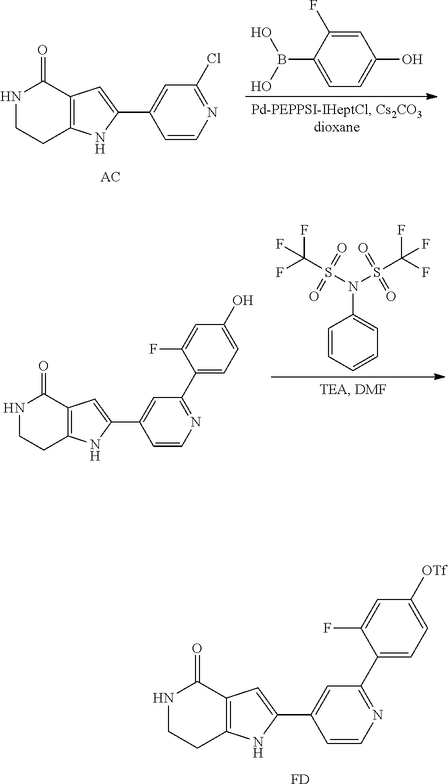

Step 1—2-(2-(2-fluoro-4-hydroxyphenyl)pyridin-4-yl)-6,7-dihydro-1H-pyrrolo[3,2-c]pyridin-4(5H)-one. A mixture of 2-(2-chloro-4-pyridyl)-1,5,6,7-tetrahydropyrrolo[3,2-c]pyridin-4-one (1.3 g, 5.3 mmol, Intermediate AC), (2-fluoro-4-hydroxy-phenyl)boronic acid (2.46 g, 15.7 mmol, CAS #1376989-43-3), 1,3-bis[2,6-bis(1-propylbutyl)phenyl]-4,5-dichloro-2H-imidazol-1-ium-2-ide 3-chloropyridine dichloropalladium (510 mg, 525 umol), and Cs₂CO₃ (5.13 g, 15.8 mmol) in dioxane (80 mL) and H₂O (20 mL) degassed and purged with N₂ three times. Then the mixture was stirred at 80° C. for 2 hrs under N₂ atmosphere. On completion, the reaction mixture was filtered and concentrated under reduced pressure to give title compound (3.0 g) as yellow solid. LC-MS (ESI+) m/z 324.0. (M+H)⁺.

Step 2—3-Fluoro-4-(4-(4-oxo-4,5,6,7-tetrahydro-1H-pyrrolo[3,2-c]pyridin-2-yl)pyridin-2-yl)phenyl trifluoromethanesulfonate. A mixture of 2-(2-(2-fluoro-4-hydroxyphenyl)pyridin-4-yl)-6,7-dihydro-1H-pyrrolo[3,2-c]pyridin-4(5H)-one (2.9 g, 8.97 mmol), 1,1,1-trifluoro-N-phenyl-N-(trifluoromethylsulfonyl)methanesulfonamide (5.13 g, 14.4 mmol), and TEA (1.82 g, 17.9 mmol, 2.50 mL) in DMF (20 mL) was stirred at 50° C. for 3 hrs. On completion, the reaction mixture was quenched with H₂O (20 mL) at 25° C., and then diluted with EA (60 mL) and extracted with EA (50 mL×2). The combined organic layers were washed with aqueous NaCl (20 mL×2), dried over Na₂SO₄, filtered and concentrated under reduced pressure to give a residue. The residue was purified by column chromatography (SiO₂, DCM:MeOH=10:1) to give the title compound (1.3 g, 29% yield) as white solid. LC-MS (ESI+) m/z 324.0. (M+H)⁺.

### 1-(3-Fluoro-4-(4-(4-oxo-4,5,6,7-tetrahydro-1H-pyrrolo[3,2-c]pyridin-2-yl)pyridin-2-yl)phenyl)piperidine-4-carbaldehyde (Intermediate FE)

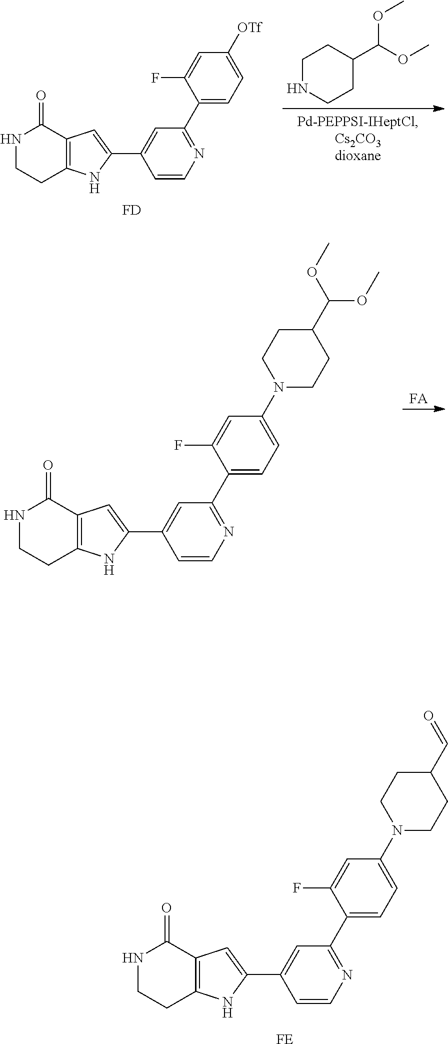

Step 1—2-(2-(4-(4-(Dimethoxymethyl)piperidin-1-yl)-2-fluorophenyl)pyridin-4-yl)-6,7-dihydro-1H-pyrrolo[3,2-c]pyridin-4(5H)-one. To a solution of 3-fluoro-4-(4-(4-oxo-4,5,6,7-tetrahydro-1H-pyrrolo[3,2-c]pyridin-2-yl)pyridin-2-yl)phenyl trifluoromethanesulfonate (50 mg, 109 umol, Intermediate FD), 4-(dimethoxymethyl)piperidine (34.9 mg, 219 umol), 1,3-bis[2,6-bis(1-propylbutyl)phenyl]-4,5-dichloro-2H-imidazol-1-ium-2-ide 3-chloropyridine dichloropalladium (10.7 mg, 10.9 umol), Cs₂CO₃ (107 mg, 329 umol) were taken up into a microwave tube in dioxane (2 mL). The sealed tube was heated at 130° C. for 2 hrs under microwave. On completion, the reaction mixture was concentrated under reduced pressure to remove solvent. The residue was purified by column chromatography (SiO₂, DCM:MeOH=20:1 to 10:1) to give the title compound (40 mg, 63% yield) as a white solid. LC-MS (ESI+) m/z 465.3. (M+H)⁺.

Step 2—1-(3-Fluoro-4-(4-(4-oxo-4,5,6,7-tetrahydro-1H-pyrrolo[3,2-c]pyridin-2-yl)pyridin-2-yl)phenyl)piperidine-4-carbaldehyde. A mixture of 2-(2-(4-(4-(dimethoxymethyl)piperidin-1-yl)-2-fluorophenyl)pyridin-4-yl)-6,7-dihydro-1H-pyrrolo[3,2-c]pyridin-4(5H)-one (500 mg, 1.08 mmol) in formic acid (10 mL) was stirred at 25° C. for 0.5 hr. On completion, the reaction mixture was concentrated under reduced pressure to give the title compound (400 mg, FA salt) as a yellow oil. LC-MS (ESI+) m/z 419.1. (M+H)⁺.

### 3-(3-methyl-2-oxo-4-(piperidin-4-yl)-2,3-dihydro-1H-benzo[d]imidazol-1-yl)piperidine-2,6-dione (Intermediate FF)

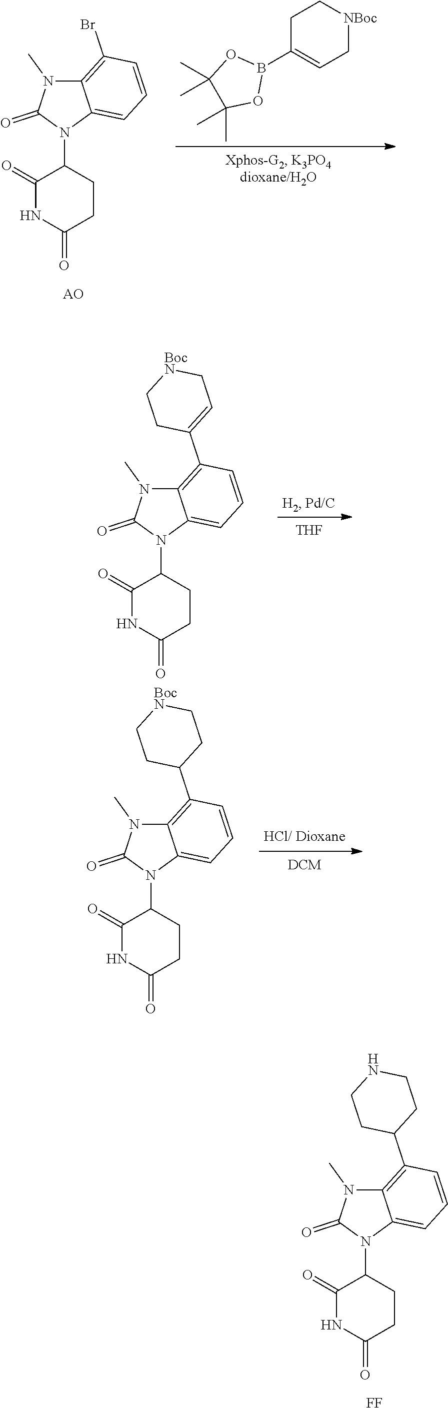

Step 1—Tert-butyl 4-(1-(2,6-dioxopiperidin-3-yl)-3-methyl-2-oxo-2,3-dihydro-1H-benzo[d]imidazol-4-yl)-5,6-dihydropyridine-1(2H)-carboxylate. A mixture of 3-(4-bromo-3-methyl-2-oxo-benzimidazol-1-yl)piperidine-2,6-dione (2.5 g, 7.4 mmol, Intermediate AO), tert-butyl 4-(4,4,5,5-tetramethyl-1,3,2-dioxaborolan-2-yl)-3,6-dihydro-2H-pyridine-1-carboxylate (3.43 g, 11.1 mmol, CAS #286961-14-6), K₃PO₄ (4.71 g, 22.2 mmol) and XPHOS-Pd-G2 (582 mg, 739 umol) in dioxane (50 mL) and H₂O (5 mL) was degassed and purged with N₂ three times. Then the mixture was stirred at 80° C. for 12 hrs under N₂ atmosphere. On completion, the reaction mixture was filtered under reduced pressure to give a residue. The residue was diluted with H₂O (100 mL) and extracted with EA (100 mL×2). The combined organic layers were washed with aqueous NaCl (100 mL×2), dried over Na₂SO₄, filtered and concentrated under reduced pressure to give a residue. The residue was triturated with EA at 20° C. for 10 min to give the title compound (2.2 g, 83% yield) as an off-white solid. LC-MS (ESI⁺) m/z 441.1 (M+H)⁺.

Step 2—Tert-butyl 4-(1-(2,6-dioxopiperidin-3-yl)-3-methyl-2-oxo-2,3-dihydro-1H-benzo[d]imidazol-4-yl)piperidine-1-carboxylate. To a solution of tert-butyl 4-(1-(2,6-dioxopiperidin-3-yl)-3-methyl-2-oxo-2,3-dihydro-1H-benzo[d]imidazol-4-yl)-5,6-dihydropyridine-1(2H)-carboxylate (1.2 g, 2.72 mmol) in THF (20 mL) was added Pd/C (250 mg, 10 wt %) and Pd(OH)₂ (250 mg, 1.78 mmol) under N₂. The suspension was degassed under vacuum and purged with H₂ several times. The mixture was stirred under H₂ (45 psi) at 30° C. for 48 hrs. On completion, the reaction mixture was filtered and concentrated under reduced pressure to give the title compound (1.1 g) as an off-white solid. LC-MS (ESI⁺) m/z 465.1 (M+Na)⁺.

Step 3—3-(3-Methyl-2-oxo-4-(piperidin-4-yl)-2,3-dihydro-1H-benzo[d]imidazol-1-yl)piperidine-2,6-dione. To a solution of tert-butyl 4-(1-(2,6-dioxopiperidin-3-yl)-3-methyl-2-oxo-2,3-dihydro-1H-benzo[d]imidazol-4-yl)piperidine-1-carboxylate (500 mg, 1.13 mmol) in DCM (10 mL) was added HCl/dioxane (4 M, 2.8 mL). The mixture was stirred at 20° C. for 0.5 hrs. On completion, the reaction mixture was concentrated under reduced pressure to give the title compound (400 mg, HCl salt) as an off-white solid. LC-MS (ESI⁺) m/z 342.9 (M+H)⁺.

### (1-(3-(4,4,5,5-tetramethyl-1,3,2-dioxaborolan-2-yl)phenyl)piperidin-4-yl)methanol (Intermediate FG)

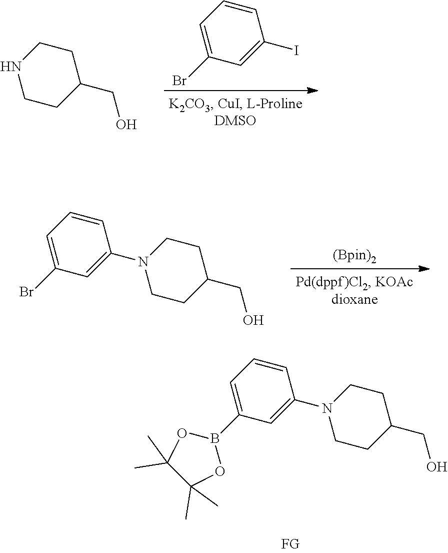

Step 1—((1-(3-Bromophenyl)piperidin-4-yl)methanol. A mixture of 1-bromo-3-iodo-benzene (1 g, 3.53 mmol, CAS #591-18-4), 4-piperidylmethanol (407.11 mg, 3.53 mmol, CAS #6457-49-4), K₂CO₃ (977 mg, 7.07 mmol), CuI (135 mg, 707 umol) and L-proline (163 mg, 1.41 mmol) in DMSO (20) was degassed and purged with N₂ three times. Then the mixture was stirred at 90° C. for 12 hrs under N₂ atmosphere. On completion, the reaction mixture was diluted with H₂O (100 mL) and extracted with EA (100 mL×3). The combined organic layers were washed with aqueous NaCl (100 mL×3), dried over Na₂SO₄, filtered and concentrated under reduced pressure to give a residue. The residue was purified by column chromatography (SiO₂, Petroleum ether/Ethyl acetate=10/1 to 3/1) to give the title compound (400 mg, 38% yield) as a yellow oil. ¹H NMR (400 MHz, CDCl₃-d) δ=7.08-6.96 (m, 2H), 6.90-6.73 (m, 2H), 3.68-3.56 (m, 2H), 3.47 (d, J=6.4 Hz, 2H), 2.72-2.58 (m, 2H), 1.77 (d, J=12.8 Hz, 2H), 1.67-1.52 (m, 1H), 1.50-1.40 (m, 1H), 1.36-1.23 (m, 2H); LC-MS (ESI⁺) m/z 269.9 (M+H)⁺.

Step 2—(1-(3-(4,4,5,5-Tetramethyl-1,3,2-dioxaborolan-2-yl)phenyl)piperidin-4-yl)methanol. A mixture of (1-(3-bromophenyl)piperidin-4-yl)methanol (400 mg, 1.48 mmol), 4,4,5,5-tetramethyl-2-(4,4,5,5-tetramethyl-1,3,2-dioxaborolan-2-yl)-1,3,2-dioxaborolane (752 mg, 2.96 mmol), KOAc (436 mg, 4.44 mmol), and Pd(dppf)Cl₂ (108 mg, 148 umol) in dioxane (10 mL) was degassed and purged with N₂ three times. Then the mixture was stirred at 80° C. for 12 hrs under N₂ atmosphere. On completion, the reaction mixture was filtered and concentrated under reduced pressure to give a residue. The residue was purified by column chromatography (SiO₂, Petroleum ether/Ethyl acetate=10/1 to 1/1) to give the title compound (210 mg, 43% yield) as a yellow solid. LC-MS (ESI⁺) m/z 317.9 (M+H)⁺.

### (R)-3-(3-(4-(hydroxymethyl)piperidin-1-yl)phenyl)-10-methyl-9,10,11,12-tetrahydro-8H-[1,4]diazepino[5′,6′:4,5]thieno[3,2-f]quinolin-8-one (Intermediate FH)

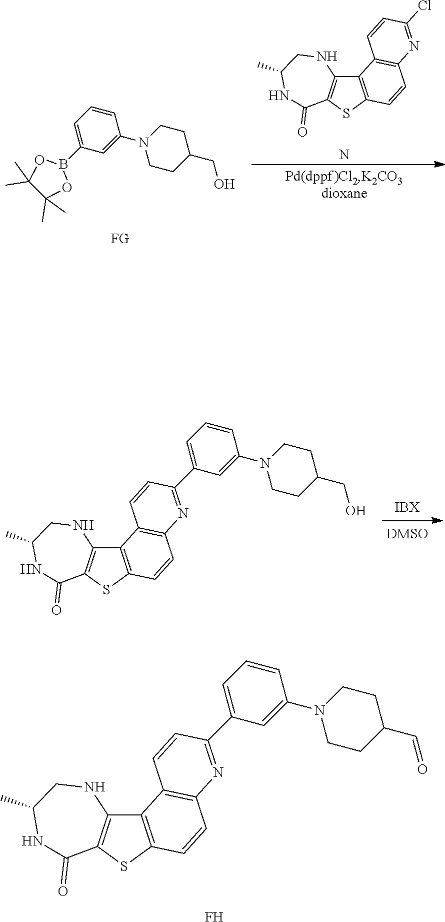

Step 1—(R)-3-(3-(4-(hydroxymethyl)piperidin-1-yl)phenyl)-10-methyl-9,10,11,12-tetrahydro-8H-[1,4]diazepino[5′,6′:4,5]thieno[3,2-f]quinolin-8-one. A mixture of (1-(3-(4,4,5,5-tetramethyl-1,3,2-dioxaborolan-2-yl)phenyl)piperidin-4-yl)methanol (200 mg, 630 umol, Intermediate FG), (R)-3-chloro-10-methyl-9,10,11,12-tetrahydro-8H-[1,4]diazepino[5′,6′:4,5]thieno[3,2-f]quinolin-8-one (200 mg, 630 umol, Intermediate N), K₂CO₃ (261 mg, 1.89 mmol), and Pd(dppf)Cl₂ (92.3 mg, 126 umol) in dioxane (5 mL) and H₂O (1 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=10/1 to DCM/MeOH=10/1) to give the title compound (230 mg, 67% yield) as an orange solid. LC-MS (ESI⁺) m/z 473.3 (M+H)⁺.

Step 2—(R)-3-(3-(4-(hydroxymethyl)piperidin-1-yl)phenyl)-10-methyl-9,10,11,12-tetrahydro-8H-[1,4]diazepino[5′,6′:4,5]thieno[3,2-f]quinolin-8-one. To a solution of (R)-3-(3-(4-(hydroxymethyl)piperidin-1-yl)phenyl)-10-methyl-9,10,11,12-tetrahydro-8H-[1,4]diazepino[5′,6′:4,5]thieno[3,2-f]quinolin-8-one (200 mg, 423 umol) in DMSO (5 mL) was added IBX (237 mg, 846 umol). The mixture was stirred at 20° C. for 1 hrs. On completion the reaction mixture was diluted with H₂O (20 mL) and extracted with EA (20 mL×3). The combined organic layers were washed with aqueous NaCl (20 mL×4), dried over Na₂SO₄, filtered and concentrated under reduced pressure give the title compound (200 mg) as a red solid. LC-MS (ESI⁺) m/z 471.3 (M+H).

### 3-(1-oxo-5-(piperazin-1-yl)isoindolin-2-yl)piperidine-2,6-dione (Intermediate FI)

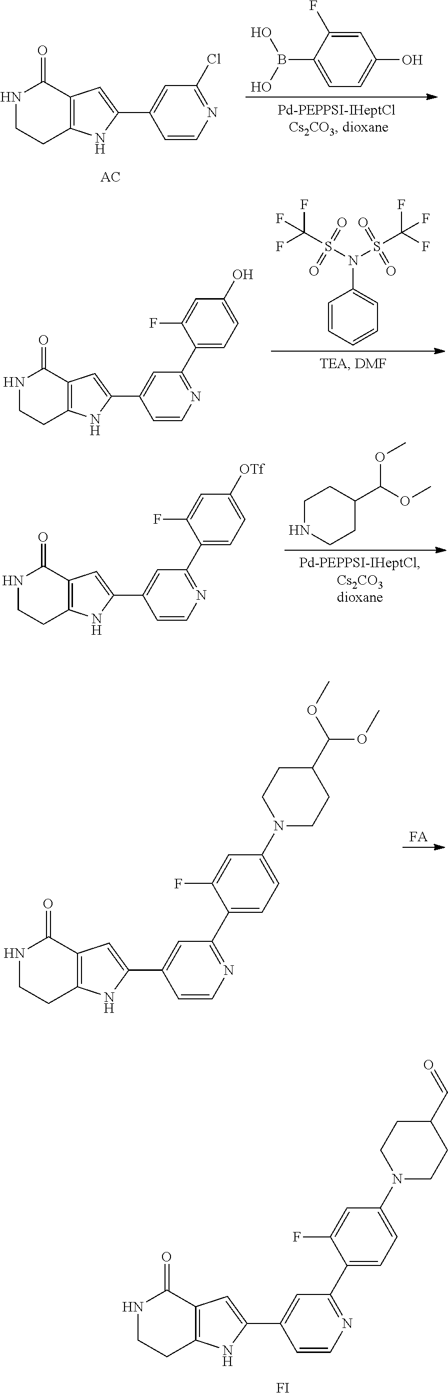

Step 1 2-(2-(2-fluoro-4-hydroxyphenyl)pyridin-4-yl)-6,7-dihydro-1H-pyrrolo[3,2-c]pyridin-4(5H)-one. A mixture of 2-(2-chloro-4-pyridyl)-1,5,6,7-tetrahydropyrrolo[3,2-c]pyridin-4-one (1.3 g, 5.25 mmol, Intermediate AC), (2-fluoro-4-hydroxy-phenyl)boronic acid (2.46 g, 15.7 mmol, CAS #1376989-43-3), 1,3-bis[2,6-bis(1-propylbutyl)phenyl]-4,5-dichloro-2H-imidazol-1-ium-2-ide;3-chloropyridine dichloropalladium (510 mg, 525 umol), Cs₂CO₃ (5.13 g, 15.8 mmol) in dioxane (80 mL) and H₂O (20 mL) was degassed and purged with N₂ three times. Then the mixture was stirred at 80° C. for 2 hours under N₂ atmosphere. On completion, the reaction mixture was filtered and the filtrate was concentrated under reduced pressure to give the title compound (3.0 g) as a yellow solid. LC-MS (ESI+) m/z 324.0. (M+H)⁺.

Step 2—3-Fluoro-4-(4-(4-oxo-4,5,6,7-tetrahydro-1H-pyrrolo[3,2-c]pyridin-2-yl)pyridin-2-yl)phenyl trifluoromethanesulfonate. A mixture of 2-(2-(2-fluoro-4-hydroxyphenyl)pyridin-4-yl)-6,7-dihydro-1H-pyrrolo[3,2-c]pyridin-4(5H)-one (2.9 g, 8.97 mmol), 1,1,1-trifluoro-N-phenyl-N-(trifluoromethylsulfonyl)methanesulfonamide (5.13 g, 14.4 mmol, CAS #37595-74-7), and TEA (1.82 g, 17.9 mmol) in DMF (20 mL) was stirred at 50° C. for 3 hrs. On completion, the reaction mixture was quenched with H₂O (20 mL) at 25° C., and then diluted with EA (60 mL) and extracted with EA 100 mL (50 mL×2). The combined organic layers were washed with brine (20 mL×2), dried over Na₂SO₄, filtered and the filtrate was concentrated under reduced pressure to give a residue. The residue was purified by column chromatography (SiO₂, DCM:MeOH=10:1) to give the title compound (1.3 g, 29% yield) as a white solid. LC-MS (ESI+) m/z 456.1. (M+H)⁺.

Step 3 2-(2-(4-(4-(dimethoxymethyl)piperidin-1-yl)-2-fluorophenyl)pyridin-4-yl)-6,7-dihydro-1H-pyrrolo[3,2-c]pyridin-4(5H)-one. A solution of 3-fluoro-4-(4-(4-oxo-4,5,6,7-tetrahydro-1H-pyrrolo[3,2-c]pyridin-2-yl) pyridin-2-yl)phenyl trifluoromethanesulfonate (50 mg, 109 umol), 4-(Dimethoxymethyl)piperidine (34.9 mg, 219 umol, CAS #188646-83-5), 1,3-bis[2,6-bis(1-propylbutyl)phenyl]-4,5-dichloro-2H-imidazol-1-ium-2-ide;3-chloropyridine; dichloropalladium (10.7 mg, 10.9 umol), Cs₂CO₃ (107 mg, 329 umol) were taken up into a microwave tube in dioxane (2 mL). The sealed tube was heated at 130° C. for 2 hrs under microwave. On completion, the reaction mixture was concentrated under reduced pressure to remove solvent. The residue was purified by column chromatography (SiO₂, DCM:MeOH=20:1 to 10:1) to give the title compound (40 mg, 63% yield) as a white solid. LC-MS (ESI+) m/z 465.3. (M+H)⁺.

Step 4—1-(3-Fluoro-4-(4-(4-oxo-4,5,6,7-tetrahydro-1H-pyrrolo[3,2-c]pyridin-2-yl)pyridin-2-yl)phenyl)piperidine-4-carbaldehyde. A mixture of 2-(2-(4-(4-(dimethoxymethyl)piperidin-1-yl)-2-fluorophenyl)pyridin-4-yl)-6,7-dihydro-1H-pyrrolo[3,2-c]pyridin-4(5H)-one (500 mg, 1.08 mmol) in formic acid (10 mL) was stirred at 25° C. for 0.5 hrs. On completion, the reaction mixture was concentrated under reduced pressure to give the title compound (400 mg, crude, FA salt) as a yellow oil. LC-MS (ESI+) m/z 419.1. (M+H)⁺.

### 2-[1-[4-[(15R)-15-methyl-13-oxo-11-thia-6,14,17-triazatetracyclo[8.8.0.0^{2,7}.0^{12,18}]octadeca-1,3,5,7,9,12(18)-hexaen-5-yl]phenyl]-4-piperidyl]acetaldehyde (Intermediate FJ)

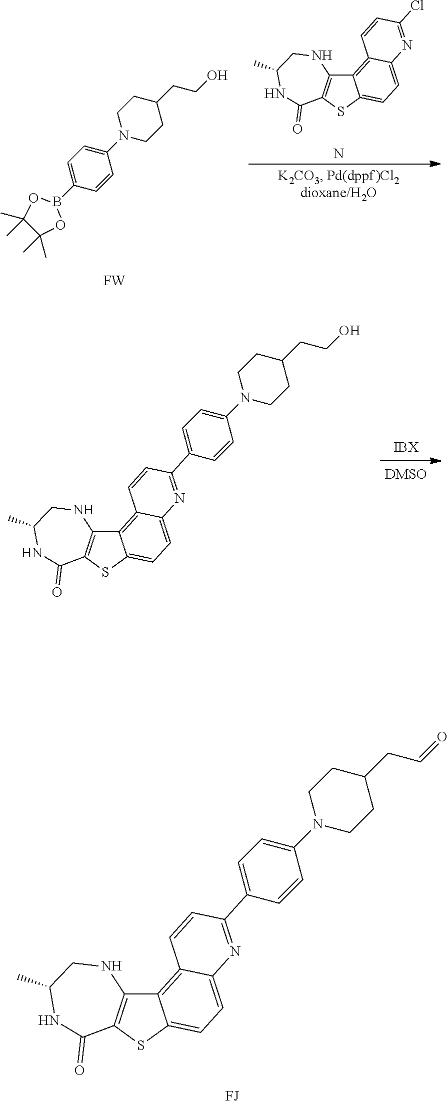

Step 1—(R)-3-(4-(4-(2-hydroxyethyl)piperidin-1-yl)phenyl)-10-methyl-9,10,11,12-tetrahydro-8H-[1,4]diazepino[5′,6′:4,5]thieno[3,2-f]quinolin-8-one. To a solution of (15R)-5-chloro-15-methyl-11-thia-6,14,17-triazatetracyclo[8.8.0.0^{2,7}.0^{12,18}]octadeca-1,3,5,7,9,12(18)-hexaen-13-one (500 mg, 1.57 mmol, Intermediate N) and 2-[1-[4-(4,4,5,5-tetramethyl-1,3,2-dioxaborolan-2-yl)phenyl]-4-piperidyl]ethanol (678 mg, 2.05 mmol, Intermediate FW) in dioxane (5 mL) and H₂O (1 mL) was added K₂CO₃ (652 mg, 4.72 mmol) and Pd(dppf)Cl₂ (115 mg, 157 umol). The mixture was stirred at 80° C. for 2 hrs. On completion, the reaction mixture was diluted with H₂O (15 mL) and extracted with EA (15 mL×3). The combined organic layers were dried over Na₂SO₄, filtered and the filtrate was concentrated under reduced pressure to give a residue. The residue was purified by column chromatography (SiO2, Petroleum ether/Ethyl acetate=1/0 to 0/1) to give the title compound (420 mg, 27% yield) as a yellow solid. LC-MS (ESI⁺) m/z 487.1 (M+1)⁺.

Step 2—(R)-2-(1-(4-(10-methyl-8-oxo-9,10,11,12-tetrahydro-8H-[1,4]diazepino[5′,6′:4,5]thieno[3,2-f]quinolin-3-yl)phenyl)piperidin-4-yl)acetaldehyde. To a solution of (15R)-5-[4-[4-(2-hydroxyethyl)-1-piperidyl]phenyl]-15-methyl-11-thia-6,14,17-triazatetracyclo[8.8.0.0^{2,7}.0^{12,18}]octadeca-1,3,5,7,9,12(18)-hexaen-13-one (200 mg, 411 umol) in DMSO (5 mL) was added IBX (230 mg, 822 umol). The mixture was stirred at 25° C. for 1 hr. On completion, the reaction mixture was diluted with H₂O (50 mL) and extracted with EA (40 mL×4). The combined organic layers were washed with brine (40 mL×4), dried over Na₂SO₄, filtered and the filtrate was concentrated under reduced pressure to give the title compound (150 mg) as a red solid. LC-MS (ESI⁺) m/z 485.1 (M+1)⁺.

### 1-(1-(2,6-Dioxopiperidin-3-yl)-3-methyl-2-oxo-2,3-dihydro-1H-benzo[d]imidazol-4-yl)azetidine-3-carbaldehyde (Intermediate FK)

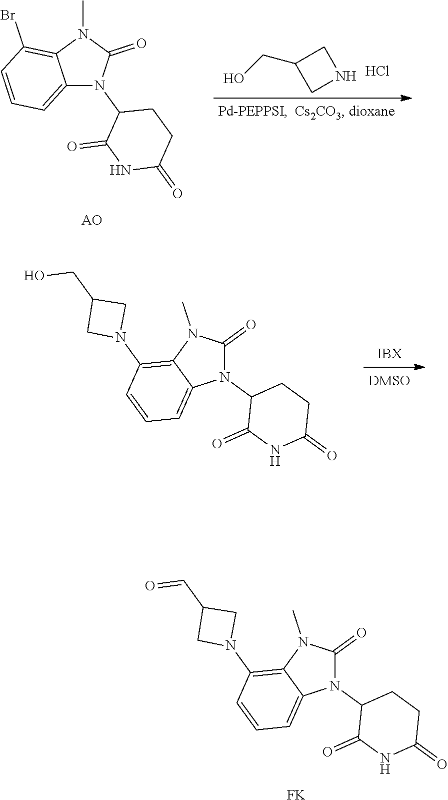 [SMILES: Cn1c(=O)n(C2CCC(=O)NC2=O)c2cccc(Br)c21 ; OCC1CNC1 ; Cl ; Cn1c(=O)n(C2CCC(=O)NC2=O)c2cccc(N3CC(CO)C3)c21 ; Cn1c(=O)n(C2CCC(=O)NC2=O)c2cccc(N3CC(C=O)C3)c21 ; "FK"]

Step 1 3-(4-(3-(hydroxymethyl)azetidin-1-yl)-3-methyl-2-oxo-2,3-dihydro-1H-benzo[d]imidazol-1-yl)piperidine-2,6-dione. To a solution of 3-(4-bromo-3-methyl-2-oxo-2,3-dihydro-1H-benzo[d]imidazol-1-yl)piperidine-2,6-dione (200 mg, 591 umol, Intermediate AO) and azetidin-3-ylmethanol (110 mg, 887 umol, HCl, CAS #95849-02-8) in dioxane (5 mL) was added Pd-PEPPSI-IHeptCl (57.5 mg, 59.1 umol, CAS #1814936-54-3) and Cs₂CO₃ (578 mg, 1.77 mmol) at 25° C., then the reaction was stirred at 100° C. for 12 hrs. On completion, the reaction mixture was filtered and the filtrate was concentrated in vacuo to give the crude residue. The crude product was purified by reversed-phase HPLC (0.1% FA condition) to give the title compound (150 mg, 22% yield, FA) as an off-white solid. LC-MS (ESI+) m/z 345.0. (M+H)⁺.

Step 2 1-(1-(2,6-Dioxopiperidin-3-yl)-3-methyl-2-oxo-2,3-dihydro-1H-benzo[d]imidazol-4-yl)azetidine-3-carbaldehyde. To a solution of 3-(4-(3-(hydroxymethyl)azetidin-1-yl)-3-methyl-2-oxo-2,3-dihydro-1H-benzo[d]imidazol-1-yl)piperidine-2,6-dione (140 mg, 407 umol) in DMSO (5 mL) was added IBX (228 mg, 813 umol) at 25° C., then the mixture was stirred at 25° C. for 5 hrs. On completion, the reaction mixture was quenched with water (20 mL) and extracted with ethyl acetate (3×20 mL). The combined organic layers were washed with brine (20 mL), dried over anhydrous sodium sulfate, then filtered. The filtrate was concentrated in vacuo to give the title compound (200 mg) as a brown solid. LC-MS (ESI+) m/z 361.3 (M+18)⁺.

### 3-(4-bromo-5-methoxy-3-methyl-2-oxo-benzimidazol-1-yl)piperidine-2,6-dione (Intermediate FL)

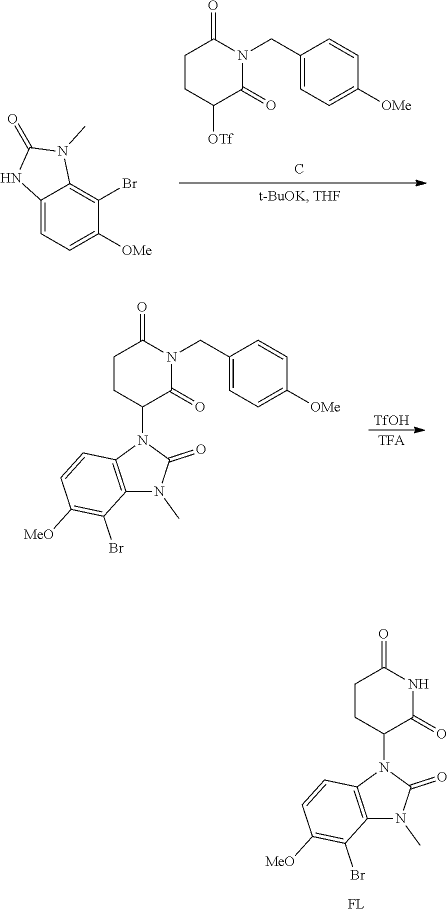

Step 1—3-(4-Bromo-5-methoxy-3-methyl-2-oxo-benzimidazol-1-yl)-1-[(4-methoxyphenyl)methyl] piperidine-2,6-dione. To a mixture of [1-[(4-methoxyphenyl)methyl]-2,6-dioxo-3-piperidyl] trifluoromethanesulfonate (2.22 g, 5.83 mmol, Intermediate C) and t-BuOK (654 mg, 5.83 mmol) in THF (20 mL) was added 4-bromo-5-methoxy-3-methyl-1H-benzimidazol-2-one (1.00 g, 3.89 mmol, synthesized via Steps 1-4 of Intermediate HG) at 0° C. Then the mixture was warmed to rt and stirred for 12 hours. On completion, the mixture was poured into the water (30 mL) and extracted with ethyl acetate (2×20 mL). The combined organic phase was washed with brine (2×30 mL), dried with anhydrous Na₂SO₄, filtered and concentrated in vacuo to give the title compound (1.50 g, 78% yield) as a brown solid. ¹H NMR (400 MHz, CDCl₃) δ 7.39-7.33 (m, 2H), 6.85-6.82 (m, 2H), 6.50 (d, J=8.4 Hz, 1H), 6.32 (d, J=8.4 Hz, 1H), 5.19 (dd, J=5.6, 13.2 Hz, 1H), 4.96 (s, 2H), 3.86 (s, 3H), 3.80 (s, 6H), 3.05-2.96 (m, 1H), 2.88-2.76 (m, 1H), 2.58 (dd, J=4.4, 13.2 Hz, 1H), 2.19-2.15 (m, 1H).

Step 2—3-(4-bromo-5-methoxy-3-methyl-2-oxo-benzimidazol-1-yl)piperidine-2,6-dione. To a solution of 3-(4-bromo-5-methoxy-3-methyl-2-oxo-benzimidazol-1-yl)-1-[(4-methoxyphenyl)methyl]piperidine-2,6-dione (3.00 g, 6.14 mmol) in TFA (36 mL) was added TfOH (1.8 mL). The mixture was then stirred at 65° C. for 3 hours. On completion, the mixture was concentrated to give a residue, then the residue was adjusted pH to 6-7 by TEA at 0° C. The mixture was concentrated to give a residue. The crude product was purified by reversed-phase HPLC (0.1% FA condition) to give the title (600 mg, 26% yield) as a gray solid. H NMR (400 MHz, DMSO-d₆) δ=11.11 (s, 1H), 7.97-7.89 (m, 1H), 7.14-7.06 (m, 1H), 7.05-7.01 (m, 1H), 6.82 (d, J=8.6 Hz, 1H), 5.45-5.26 (m, 1H), 3.64 (s, 4H), 2.97-2.81 (m, 2H), 2.39-2.29 (m, 1H), 2.06-1.97 (m, 1H). LC-MS (ESI⁺) m/z 370.0 (M+H)⁺.

### 3-(3-methyl-2-oxo-4-(2-(piperazin-1-yl)ethyl)-2,3-dihydro-1H-benzo[d]imidazol-1-yl)piperidine-2,6-dione (Intermediate FM)

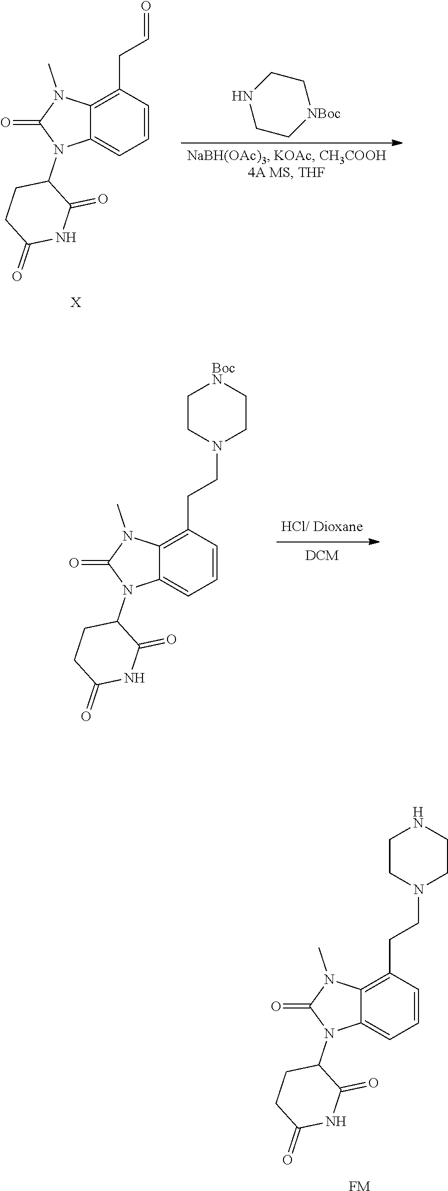

Step 1—Tert-butyl 4-(2-(1-(2,6-dioxopiperidin-3-yl)-3-methyl-2-oxo-2,3-dihydro-1H-benzo[d]imidazol-4-yl)ethyl)piperazine-1-carboxylate. To a solution of tert-butyl piperazine-1-carboxylate (1.21 g, 6.47 mmol) in THF (2 mL) was added KOAc (733 mg, 7.47 mmol). The mixture was stirred at 25° C. for 0.5 hr, then was added 2-[1-(2,6-dioxo-3-piperidyl)-3-methyl-2-oxo-benzimidazol-4-yl]acetaldehyde (1.5 g, 4.98 mmol, Intermediate X) and 4 Å molecular sieves (2 g) and the mixture was stirred at 25° C. for 0.5 hr. Then NaBH(OAc)₃ (2.64 g, 12.5 mmol) was added and the mixture was stirred at 0° C. for 1 hr. On completion, the reaction mixture was filtered and concentrated under reduced pressure to give a filtrate. The filtrate was purified by reversed-phase HPLC (0.1% FA condition) to give the title compound (900 mg, 37% yield) as a white solid. LC-MS (ESI⁺) m/z 472.1 (M+H)⁺.

Step 2—3-(3-Methyl-2-oxo-4-(2-(piperazin-1-yl)ethyl)-2,3-dihydro-1H-benzo[d]imidazol-1-yl)piperidine-2,6-dione. To a solution of tert-butyl 4-[2-[1-(2,6-dioxo-3-piperidyl)-3-methyl-2-oxo-benzimidazol-4-yl]ethyl]piperazine-1-carboxylate (200 mg, 424 umol) in DCM (18 mL) was added HCl/dioxane (1 M, 6 mL). The mixture was stirred at 25° C. for 1 hr. On completion, the reaction mixture was concentrated in vacuo to give the title product (180 mg) as a white solid. LC-MS (ESI⁺) m/z 372.1 (M+H)⁺.

### 3-(3-bromophenyl)piperidine-2,6-dione (Intermediate FN)

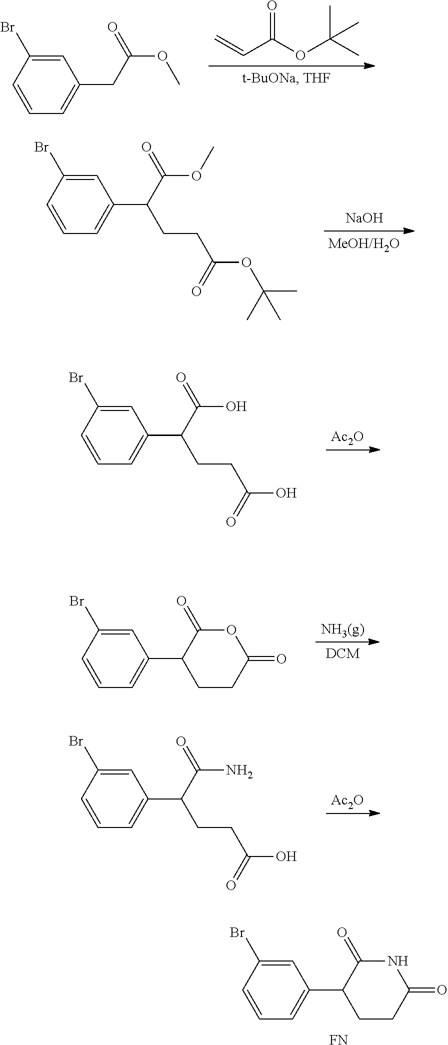

Step 1—5-Tert-butyl 1-methyl 2-(3-bromophenyl)pentanedioate. To a stirred solution of methyl 2-(3-bromophenyl)acetate (50 g, 218 mmol) and tert-butyl prop-2-enoate (28 g, 218 mmol) in THF (200 mL) was added t-BuONa (4.20 g, 43.6 mmol) at −30 C under nitrogen atmosphere. The resulting mixture was stirred for 1 h at −30° C. under nitrogen atmosphere. On completion, the reaction mixture was concentrated under vacuum. The resulting mixture was extracted with EtOAc (2×400 mL). The combined organic layers were washed with brine (2×300 mL), and dried over anhydrous Na₂SO₄. After filtration, the filtrate was concentrated under reduced pressure to afford the title compound (60 g, 77% yield) as a light yellow oil. LC/MS (ESI, m/z): [(M+H)]⁺=357.2; 359.2.

Step 2—2-(4-bromophenyl)pentanedioic acid. To a stirred mixture of 5-tert-butyl 1-methyl 2-(3-bromophenyl)pentanedioate (60 g, 168 mmol) and MeOH (300 mL) in H₂O (300 mL) was added NaOH (20.1 g, 504 mmol) in portions at 0° C. under nitrogen atmosphere. The resulting mixture was stirred for 2 h at rt under nitrogen atmosphere. On completion, the reaction mixture was concentrated under reduced pressure. The resulting mixture was diluted with water (300 mL). The residue was acidified to pH 3 with conc. HCl. The resulting mixture was extracted with EtOAc (2×300 mL). The combined organic layers were washed with brine (2×200 mL) and dried over anhydrous Na₂SO₄. After filtration, the filtrate was concentrated under reduced pressure to afford the title compound (45 g, 99% yield) as a light yellow oil. LC/MS (ESI, m/z): [(M−H)]⁻=285.0; 287.0.

Step 3—3-(3-bromophenyl)oxane-2,6-dione. 2-(3-bromophenyl)pentanedioic acid (50 g, 174 mmol) in Ac₂O (300 mL) was stirred for 10 h at 80° C. under nitrogen atmosphere. On completion, the reaction mixture was concentrated under vacuum to afford the title compound (45 g, 96% yield) as a light yellow solid. LC/MS (ESI, m/z): [(M+H)]⁺=269.0; 271.0.

Step 4—2-(4-bromophenyl)-4-carbamoylbutanoic acid. To a stirred solution of 3-(3-bromophenyl)oxane-2,6-dione (45 g, 167 mmol) in DCM (500 mL) was added NH₃(g) in MeOH (14.2 g, 836 mmol) dropwise at rt under nitrogen atmosphere. The resulting mixture was stirred for 16 h at rt under nitrogen atmosphere. The precipitated solids were collected by filtration and washed with DCM (2×100 mL). The residue was then concentrated under vacuum to afford the title compound (35 g, 73%) as a white solid. LC/MS (ESI, m/z): [(M+H)]⁺=286.0, 288.0.

Step 5: 3-(3-bromophenyl)piperidine-2,6-dione. 4-(3-bromophenyl)-4-carbamoylbutanoic acid (25 g, 87.4 mmol) in Ac₂O (200 mL) was stirred for 10 h at 60 C under nitrogen atmosphere. On completion, the resulting mixture was concentrated under vacuum. The residue was purified by trituration with Et₂O (2×200 mL) to afford the title compound (10 g, 43% yield) as a white solid. ¹H NMR (400 MHz, DMSO-d₆) δ 10.88 (s, 1H), 7.51-7.44 (m, 2H), 7.33-7.22 (m, 2H), 3.90 (dd, J=12.2, 4.9 Hz, 1H), 2.74-2.59 (m, 1H), 2.56-2.48 (m, 1H), 2.32-2.16 (m, 1H), 2.08-1.96 (m, 1H). LC/MS (ESI, m/z): [(M+H)]⁺=265.9, 267.9.

### 1-(4-Bromophenyl)-3-[(4-methoxyphenyl)methyl]hexahydropyrimidine-2,4-dione (Intermediate FO)

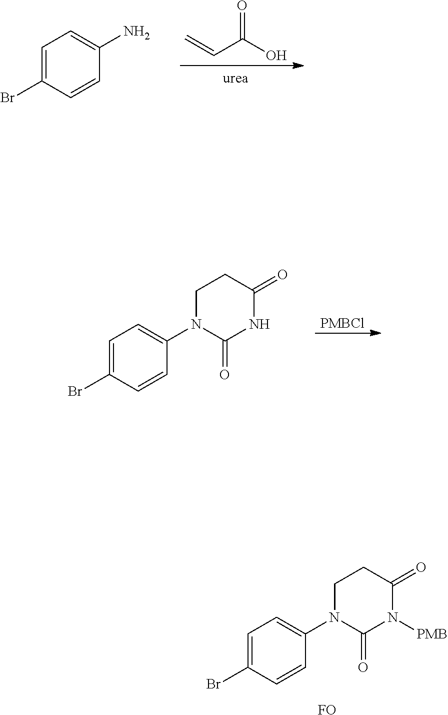

Step 1—1-(4-Bromophenyl) hexahydropyrimidine-2,4-dione. To a solution of 4-bromoaniline (40 g, 232 mmol, CAS #106-40-1) and acrylic acid (67 g, 930 mmol) was stirred at 110° C. for 3 h. Then urea (89.6 g, 1.49 mol) and AcOH (400 mL) was added into the mixture and the mixture was stirred at 120° C. for 12 h. On completion, the reaction was poured into H₂O (1000 mL) at 0° C. Then the solid was filtered, and the filter cake was washed with H₂O (500 mL) and concentrated under reduced pressure to give the title compound (40 g) as yellow solid.

Step 2—1-(4-Bromophenyl)-3-[(4-methoxyphenyl) methyl]hexahydropyrimidine-2, 4-dione. To a solution of 1-(4-bromophenyl) hexahydropyrimidine-2,4-dione (20 g, 74.3 mmol) in DMF (200 mL) was added Cs₂CO₃ (48.4 g, 148 mmol) and PMB-Cl (17.4 g, 111 mmol) at 20° C., then the mixture was stirred at 50° C. for 3 h. On completion, the reaction mixture was diluted with H₂O (200 mL) and extracted with EA (250 mL×2). The combined organic layers were washed with brine (500 mL), dried over Na₂SO₄, filtered and the filtrate was concentrated under reduced pressure to give a residue. The residue was purified by column chromatography (SiO₂, Petroleum ether/Ethyl acetate=100/1 to 2/1) to give the title compound (11.8 g, 41% yield) as white solid. ¹H NMR (400 MHz, DMSO-d₆) δ=7.59 (d, J=8.7 Hz, 2H), 7.31 (d, J=8.8 Hz, 2H), 7.23 (d, J=8.7 Hz, 2H), 6.86 (d, J=8.7 Hz, 2H), 4.81 (s, 2H), 3.80 (t, J=6.7 Hz, 2H), 3.72 (s, 3H), 2.89 (t, J=6.7 Hz, 2H).

### 3-(4-Methoxybenzyl)dihydropyrimidine-2,4(1H,3H)-dione (Intermediate FP)

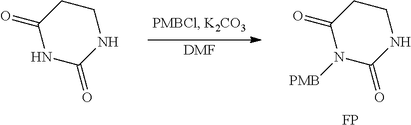

To a mixture of dihydropyrimidine-2,4(1H,3H)-dione (10.0 g, 87.6 mmol, CAS #504-07-4) in DMF (100 mL) was added PMB-Cl (13.7 g, 87.6 mmol, 11.9 mL), Cs₂CO₃ (28.5 g, 87.6 mmol) at 25° C. Then the mixture was stirred at 50° C. for 3 hours. On completion, the reaction mixture was quenched with of water (100 mL), and extracted with EtOAc (3×50 mL). The organic layer was dried over anhydrous Na₂SO₄, filtered and concentrated in vacuo. The crude product was purified by re-crystallization from EA/PE (20 mL, v/v=1/1) at 25° C. to give the title compound (9.40 g, 45% yield) as a white solid. ¹H NMR (400 MHz, CDCl₃) δ 7.81 (s, 1H), 7.18 (d, J=8.4 Hz, 2H), 6.83 (d, J=8.4 Hz, 2H), 4.72 (s, 2H), 3.72 (s, 3H), 3.23-3.20 (m, 2H), 2.63 (t, J=6.8 Hz, 2H).

### 1-(8-Chloro-4-isoquinolyl)hexahydropyrimidine-2,4-dione (Intermediate FQ)

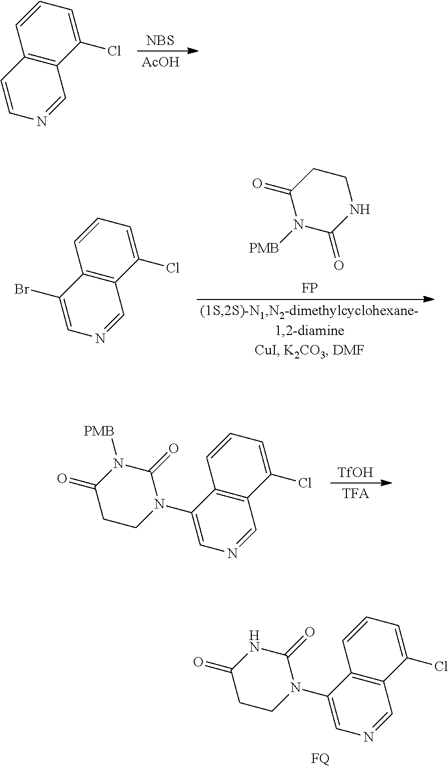

Step 1—4-Bromo-8-chloro-isoquinoline. To a solution of 8-chloroisoquinoline (5.00 g, 30.5 mmol, CAS #34784-07-1) in AcOH (50 mL) was added NBS (7.07 g, 39.7 mmol), then the reaction mixture was stirred at 50° C. for 40 min. On completion, the reaction mixture was diluted with water (100 mL), then extracted with EA (3×80 mL). The combined organic layer was basified with NaHCO₃ until the pH=6-7, then the mixture was extracted with EA (2×60 mL). The combined organic layers was dried over Na₂SO₄, filtered and concentrated in vacuo to give a residue. The residue was purified by column chromatography (SiO₂, PE:EA=100:1 to PE:EA=50:1, PE:EA=10:1, P1: R_{f}=0.74) to give the title compound (1.00 g, 37% yield) as yellow solid. ¹HNMR (400 MHz, CDCl₃) δ 9.56 (s, 1H), 8.78 (s, 1H), 8.10-8.03 (m, 1H), 7.73-7.64 (m, 2H). LC-MS (ESI⁺) m/z 241.9 (M+H)⁺.

Step 2—1-(8-Chloro-4-isoquinolyl)-3-[(4-methoxyphenyl)methyl]hexahydropyrimidine-2,4-dione. To a solution of 4-bromo-8-chloro-isoquinoline (100 mg, 412 umol) and 3-[(4-methoxyphenyl)methyl] hexahydropyrimidine-2,4-dione (96.6 mg, 412.37 umol, Intermediate FP) in DMF (1 mL) was added CuI (7.85 mg, 41.2 umol), (1S,2S)—N₁,N₂-dimethylcyclohexane-1,2-diamine (5.87 mg, 41.2 umol) and K₃PO₄ (175 mg, 824 umol), then the mixture was stirred at 110° C. for 8 hr. On completion, the reaction mixture was filtered and concentrated in vacuo to give the residue. The residue was diluted with water (50 mL) and extracted with EA (5×30 mL). Then the combined organic layers was dried over anhydrous Na₂SO₄, filtered and concentrated in vacuo to give a residue. The residue was purified by reverse-phase (0.1% FA) to give the title compound (15 mg, 3.06% yield) as yellow solid. ¹H NMR (400 MHz, DMSO-d₆) δ 9.89-9.56 (br s, 1H), 8.59 (br s, 1H), 7.73-7.68 (m, 1H), 7.64 (t, J=8.0 Hz, 1H), 7.60-7.55 (m, 1H), 7.43 (d, J=8.4 Hz, 2H), 6.85 (d, J=8.4 Hz, 2H), 5.00 (s, 2H), 3.95-3.86 (m, 1H), 3.80 (s, 3H), 3.78-3.69 (m, 1H), 3.07-2.99 (m, 2H); LC-MS (ESI⁺) m/z 396.1 (M+H)⁺.

Step 3—1-(8-Chloro-4-isoquinolyl)hexahydropyrimidine-2,4-dione. To a solution of 1-(8-chloro-4-isoquinolyl)-3-[(4-methoxyphenyl)methyl]hexahydropyrimidine-2,4-dione (40.0 mg, 101 umol) in TFA (0.49 mL) and TfOH (0.01 mL), then the mixture was stirred at 60° C. for 2 hours. On completion, the mixture was concentrated to give the residue and purified by prep-HPLC (0.1% FA) to give the title compound (3 mg, 10.77% yield) as yellow solid. ¹H NMR (400 MHz, DMSO-d₆) δ=10.59 (s, 1H), 9.56 (s, 1H), 8.71 (s, 1H), 8.03 (d, J=8.4 Hz, 1H), 7.92-7.87 (m, 1H), 7.85-7.78 (m, 1H), 4.00-3.93 (m, 1H), 3.75-3.69 (m 1H), 3.03-2.95 (m, 1H), 2.79-2.72 (m, 1H). LC-MS (ESI⁺) m/z 276.0 (M+H)⁺.

### 2-(4-(1-(4-methoxybenzyl)-2,6-dioxopiperidin-3-yl)phenoxy)ethyl methanesulfonate (Intermediate FR)

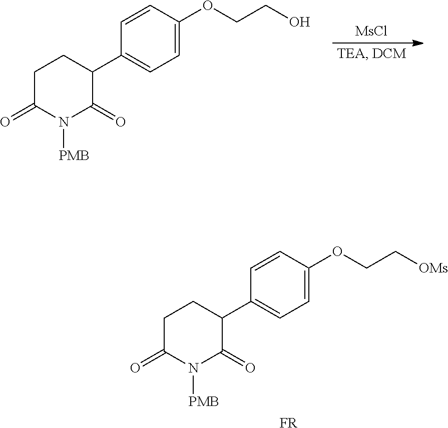

To a solution of 3-(4-(2-hydroxyethoxy)phenyl)-1-(4-methoxybenzyl)piperidine-2,6-dione (95 mg, 257 umol, synthesized via Steps 1-3 of Intermediate AW) in DCM (3 mL) was added TEA (78 mg, 771 umol), then MsCl (38.3 mg, 334 umol) was added at 0° C. Then the mixture was stirred at 20° C. for 12 hrs. The reaction mixture was quenched with H₂O (10 mL) at 0° C., and extracted with EA (10 mL×3). The combined organic layers were washed with brine (10 mL×3), dried over Na₂SO₄, filtered and the filtrate was concentrated under reduced pressure to give a residue. The residue was purified by column chromatography (SiO₂, Petroleum ether/Ethyl acetate=1/0 to 0/1) to give the title compound (55 mg, 37% yield) as a white oil. LC-MS (ESI⁺) m/z 448.1 (M+H)⁺.

### Tert-butyl 4-(3-chloro-4-(4,4,5,5-tetramethyl-1,3,2-dioxaborolan-2-yl)phenyl)piperazine-1-carboxylate (Intermediate FS)

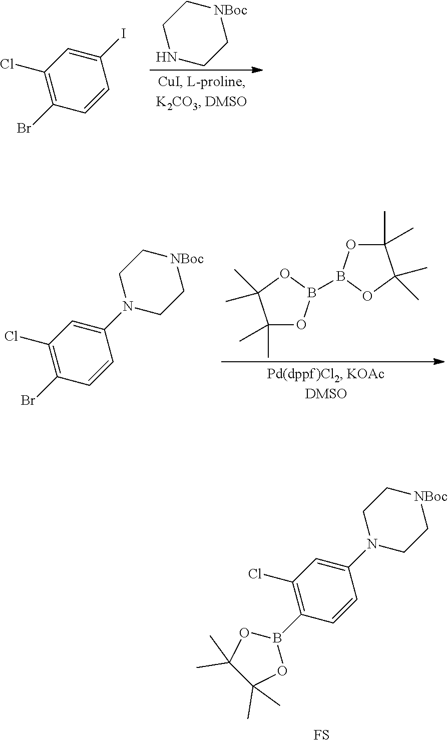

Step 1—Tert-butyl 4-(4-bromo-3-chlorophenyl)piperazine-1-carboxylate. A mixture of 1-bromo-2-chloro-4-iodobenzene (5 g, 15.7 mmol, CAS #535934-25-9), tert-butyl piperazine-1-carboxylate (2.93 g, 15.7 mmol, CAS #143238-38-4), K₂CO₃ (4.36 g, 31.5 mmol), CuI (600 mg, 3.15 mmol) and L-proline (725 mg, 6.30 mmol) in DMSO (100 mL) was degassed and purged with N₂ three times. Then the mixture was stirred at 100° C. for 2 hrs under N₂ atmosphere. On completion, the reaction mixture was diluted with H₂O (200 mL) and extracted with EA (200 mL×3). The combined organic layers were washed with brine (200 mL×3), dried over Na₂SO₄, filtered and concentrated under reduced pressure to give a residue. The residue was purified by column chromatography (SiO₂, Petroleum ether/Ethyl acetate=10/0 to 10/1) to give the title compound (2.2 g, 37% yield) as a yellow oil. LC-MS (ESI⁺) m/z 375.0 (M+H)⁺.

Step 2—Tert-butyl 4-(3-chloro-4-(4,4,5,5-tetramethyl-1,3,2-dioxaborolan-2-yl)phenyl)piperazine-1-carboxylate. A mixture of tert-butyl 4-(4-bromo-3-chlorophenyl)piperazine-1-carboxylate (1 g, 2.66 mmol), 4,4,5,5-tetramethyl-2-(4,4,5,5-tetramethyl-1,3,2-dioxaborolan-2-yl)-1,3,2-dioxaborolane (2.03 g, 7.99 mmol), KOAc (784 mg, 7.99 mmol), and Pd(dppf)Cl₂ (389 mg, 532 umol) in DMSO (30 mL) was degassed and purged with N₂ three times, and then the mixture was stirred at 110° C. for 12 hours under N₂ atmosphere. On completion, the reaction mixture was diluted with H₂O (100 mL) and extracted with EA (100 mL×2). The combined organic layers were washed with brine (100 mL×2), dried over Na₂SO₄, filtered and the filtrate was concentrated under reduced pressure to give a residue. The residue was purified by column chromatography (SiO₂, Petroleum ether/Ethyl acetate=10/0 to 10/1) to give the title compound (420 mg, 33% yield) as a yellow oil. LC-MS (ESI⁺) m/z 423.1 (M+H)⁺

### (R)-3-(2-chloro-4-(piperazin-1-yl)phenyl)-10-methyl-9,10,11,12-tetrahydro-8H-[1,4]diazepino[5′,6′:4,5]thieno[3,2-f]quinolin-8-one (Intermediate FT)

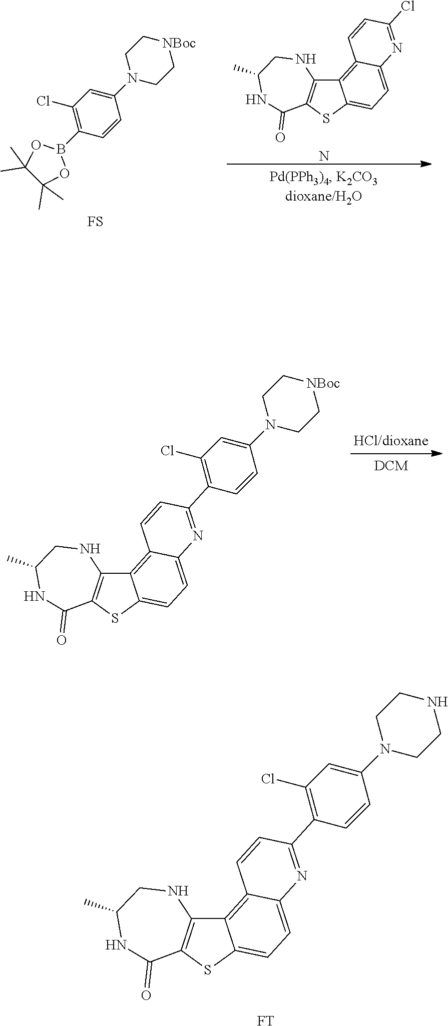

Step 1—Tert-butyl (R)-4-(3-chloro-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. A mixture of tert-butyl 4-(3-chloro-4-(4,4,5,5-tetramethyl-1,3,2-dioxaborolan-2-yl)phenyl)piperazine-1-carboxylate (400 mg, 946 umol, Intermediate FS), (R)-3-chloro-10-methyl-9,10,11,12-tetrahydro-8H-[1,4]diazepino[5′,6′:4,5]thieno[3,2-f]quinolin-8-one (400 mg, 1.26 mmol, Intermediate N), K₂CO₃ (365 mg, 2.64 mmol), and Pd(PPh₃)₄(102 mg, 88.1 umol) in dioxane (5 mL) and H₂O (1 mL) was degassed and purged with N₂ three times. Then the mixture was stirred at 80° C. for 3 hrs under N₂ atmosphere. On completion, the reaction mixture was concentrated under reduced pressure to give a residue. The residue was triturated with EA at 20° C. for 10 min. Then the solution was filtered and the filter cake was dried under vacuum to give the title compound (400 mg, 68% yield) as a yellow solid. LC-MS (ESI⁺) m/z 578.2 (M+H)⁺.

Step 2—(R)-3-(2-chloro-4-(piperazin-1-yl)phenyl)-10-methyl-9,10,11,12-tetrahydro-8H-[1,4]diazepino[5′,6′:4,5]thieno[3,2-f]quinolin-8-one. To tert-butyl (R)-4-(3-chloro-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (100 mg, 173 umol) in DCM (2 mL) was added HCl/dioxane (0.2 mL, 4 M). The mixture was stirred at 20° C. for 10 min. On completion, the reaction mixture was concentrated under reduced pressure to give the title compound (85 mg) as a red solid. LC-MS (ESI⁺) m/z 478.2 (M+H)⁺.

### Tert-butyl 4-(3-cyano-5-(4,4,5,5-tetramethyl-1,3,2-dioxaborolan-2-yl)phenyl)piperazine-1-carboxylate (Intermediate FU)

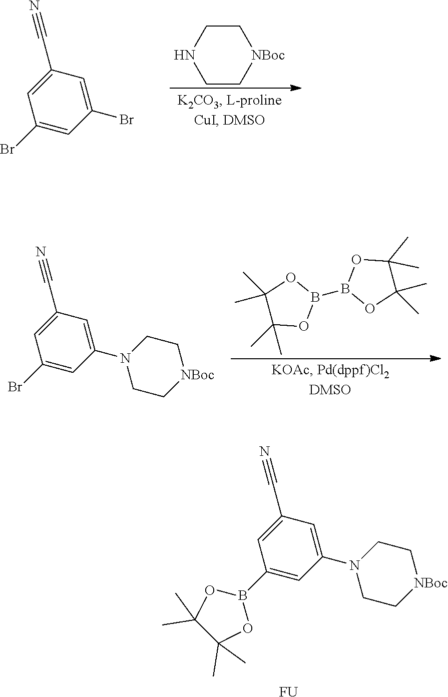

Step 1—Tert-butyl 4-(3-bromo-5-cyanophenyl)piperazine-1-carboxylate. A mixture of 3,5-dibromobenzonitrile (5 g, 19.1 mmol, CAS #9 7165-77-0), tert-butyl piperazine-1-carboxylate (3.57 g, 19.1 mmol), K₂CO₃ (5.30 g, 38.3 mmol), CuI (730 mg, 3.83 mmol) and L-proline (882 mg, 7.66 mmol) in DMSO (100 mL) was degassed and purged with N₂ three times, and then the mixture was stirred at 100° C. for 2 hrs under N₂ atmosphere. On completion, the reaction mixture was diluted with H₂O (200 mL) and extracted with EA (200 mL×3). The combined organic layers were washed with brine (200 mL×3), dried over Na₂SO₄, filtered and concentrated under reduced pressure to give a residue. The residue was purified by column chromatography (SiO2, Petroleum ether/Ethyl acetate=10/1 to 5/1) to give the title compound (1.6 g, 21% yield) as a yellow solid. LC-MS (ESI⁺) m/z 365.8 (M+H)⁺.

Step 2—Tert-butyl 4-(3-cyano-5-(4,4,5,5-tetramethyl-1,3,2-dioxaborolan-2-yl)phenyl)piperazine-1-carboxylate. A mixture of tert-butyl 4-(3-bromo-5-cyanophenyl)piperazine-1-carboxylate (600 mg, 1.64 mmol), 4,4,5,5-tetramethyl-2-(4,4,5,5-tetramethyl-1,3,2-dioxaborolan-2-yl)-1,3,2-dioxaborolane (832 mg, 3.28 mmol), KOAc (482 mg, 4.91 mmol), and Pd(dppf)Cl₂ (120 mg, 164 umol) in DMSO (15 mL) was degassed and purged with N₂ three times. Then the mixture was stirred at 80° C. for 2 hrs under N₂ atmosphere. On completion, the reaction mixture was diluted with H₂O (50 mL) and extracted with EA (50 mL×2). The combined organic layers were washed with brine (20 mL×2), dried over Na₂SO₄, filtered and the filtrate was concentrated under reduced pressure to give a residue. The residue was purified by column chromatography (SiO2, Petroleum ether/Ethyl acetate=10/1 to 5/1) to give the title compound (200 mg, 26% yield) as a yellow oil. LC-MS (ESI⁺) m/z 414.1 (M+H)⁺.

### (R)-3-(10-methyl-8-oxo-9,10,11,12-tetrahydro-8H-[1,4]diazepino[5′,6′:4,5]thieno[3,2-f]quinolin-3-yl)-5-(piperazin-1-yl)benzonitrile (Intermediate FV)

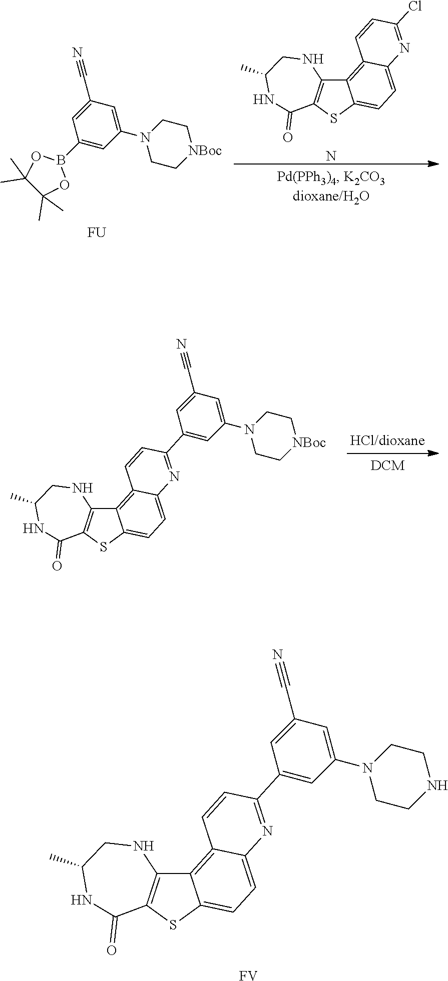

Step 1—Tert-butyl (R)-4-(3-chloro-4-(10-methyl-8-oxo-9,10,11,12-tetrahydro-8H-[1,4]diazepino[5′,6′:4,5]thieno[3,2-f]quinolin-3-yl)phenyl)piperazine-1-carboxylate. A mixture of tert-butyl 4-(3-cyano-5-(4,4,5,5-tetramethyl-1,3,2-dioxaborolan-2-yl)phenyl)piperazine-1-carboxylate (200 mg, 484 umol, Intermediate FU), (R)-3-chloro-10-methyl-9,10,11,12-tetrahydro-8H-[1,4]diazepino[5′,6′:4,5]thieno[3,2-f]quinolin-8-one (118 mg, 372 umol, Intermediate N), K₂CO₃ (154 mg, 1.12 mmol), and Pd(PPh₃)₄(43 mg, 37.2 umol) in dioxane (3 mL) and H₂O (0.5 mL) was degassed and purged with N₂ three times. Then the mixture was stirred at 80° C. for 2 hours under N₂ atmosphere. On completion, the reaction mixture was concentrated under reduced pressure to give a residue. The residue was triturated with EA at 20° C. for 10 min, the solution was filtered and the filter cake was dried under vacuum to give the title compound (200 mg, 82% yield) as a yellow solid. LC-MS (ESI⁺) m/z 569.3 (M+H)⁺.

Step 2 (R)-3-(10-methyl-8-oxo-9,10,11,12-tetrahydro-8H-[1,4]diazepino[5′,6′:4,5]thieno[3,2-f]quinolin-3-yl)-5-(piperazin-1-yl)benzonitrile. To tert-butyl (R)-4-(3-cyano-5-(10-methyl-8-oxo-9,10,11,12-tetrahydro-8H-[1,4]diazepino[5′,6′:4,5]thieno[3,2-f]quinolin-3-yl)phenyl)piperazine-1-carboxylate (100 mg, 176 umol) in DCM (2 mL) was added HCl/dioxane (0.4 mL, 4 M). The mixture was stirred at 20° C. for 10 min. On completion, the reaction mixture was concentrated under reduced pressure to give the title compound (85 mg) as a white solid. LC-MS (ESI⁺) m/z 469.3 (M+H)⁺.

### 2-(1-(4-(4,4,5,5-tetramethyl-1,3,2-dioxaborolan-2-yl)phenyl)piperidin-4-yl)ethanol (Intermediate FW)

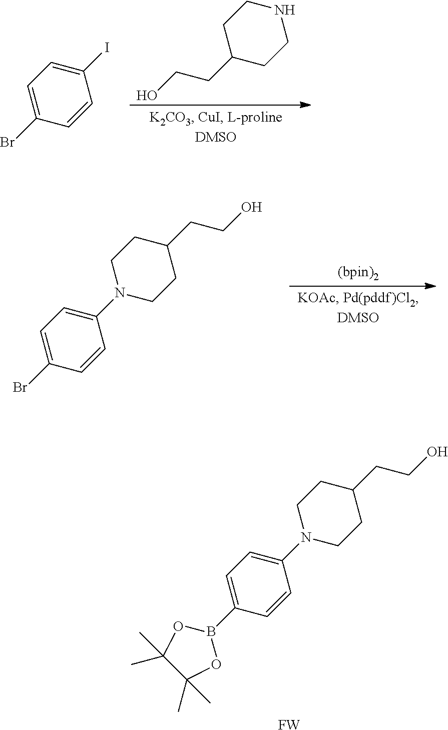 [SMILES: Brc1ccc(I)cc1 ; OCCC1CCNCC1 ; OCCC1CCN(c2ccc(Br)cc2)CC1 ; CC1(C)OB(c2ccc(N3CCC(CCO)CC3)cc2)OC1(C)C ; "FW"]

Step 1—2-(1-(4-bromophenyl)piperidin-4-yl)ethanol. A mixture of 1-bromo-4-iodo-benzene (10 g, 35.35 mmol, CAS #589-87-7), 2-(4-piperidyl)ethanol (4.57 g, 35.4 mmol, CAS #622-26-4), K₂CO₃ (9.77 g, 70.7 mmol), CuI (1.35 g, 7.07 mmol) and L-proline (1.63 g, 14.1 mmol) in DMSO (200 mL) was degassed and purged with N₂ three times. Then the mixture was stirred at 100° C. for 12 hrs under N₂ atmosphere. The reaction mixture was diluted with H₂O (200 mL) and extracted with EA (200 mL×4). The combined organic layers were washed with brine (300 mL×3), dried over Na₂SO₄, filtered and the filtrate was concentrated under reduced pressure to give a residue. The residue was purified by column chromatography (SiO₂, Petroleum ether/Ethyl acetate=1/0 to 3/1) to give the title compound (4.2 g, 28% yield) as a white solid. LC-MS (ESI⁺) m/z 283.8 (M+1)⁺.

Step 2—2-(1-(4-(4,4,5,5-tetramethyl-1,3,2-dioxaborolan-2-yl)phenyl)piperidin-4-yl)ethanol. a solution of 2-[1-(4-bromophenyl)-4-piperidyl]ethanol (2 g, 7.04 mmol) and 4,4,5,5-tetramethyl-2-(4,4,5,5-tetramethyl-1,3,2-dioxaborolan-2-yl)-1,3,2-dioxaborolane (3.57 g, 14.1 mmol) in DMSO (6 mL) was added KOAc (2.07 g, 21.1 mmol) and Pd(dppf)Cl₂ (515 mg, 704 umol). The mixture was degassed and purged with N₂ three times, then stirred at 60° C. for 4 hrs. On completion, the reaction mixture was diluted with H₂O (25 mL) and extracted with EA (20 mL×4). The combined organic layers were washed with brine (25 mL×3), dried over Na₂SO₄, filtered and the filtrate was concentrated under reduced pressure to give a residue. The residue was purified by column chromatography (SiO₂, Petroleum ether/Ethyl acetate=1/0 to 3/1) to give the title compound (1.52 g, 46% yield) as a brown solid. LC-MS (ESI⁺) m/z 331.8 (M+1)⁺.

### Tert-butyl 4-(2,6-dimethyl-4-(4,4,5,5-tetramethyl-1,3,2-dioxaborolan-2-yl)phenyl)piperazine-1-carboxylate (Intermediate FX)

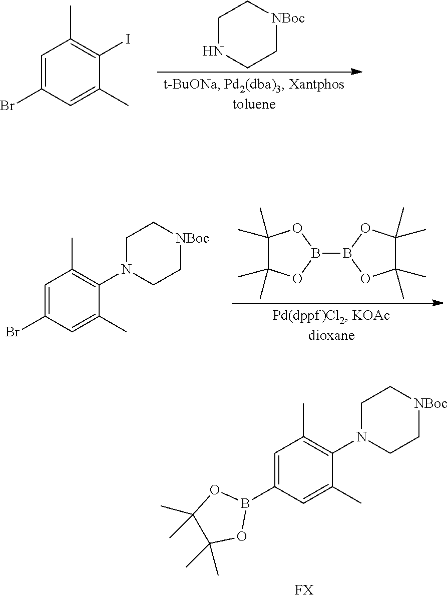

Step 1—Tert-butyl 4-(4-bromo-2,6-dimethylphenyl)piperazine-1-carboxylate. To a solution of 5-bromo-2-iodo-1,3-dimethyl-benzene (5 g, 16.1 mmol, CAS #206559-43-5) in toluene (100 mL) was added tert-butyl piperazine-1-carboxylate (2.99 g, 16.1 mmol, CAS #143238-38-4), t-BuONa (2.32 g, 24.1 mmol), and Pd₂(dba)₃ (736 mg, 804 umol) and Xantphos (930 mg, 1.61 mmol). The mixture was stirred at 60° C. for 12 hrs under N₂ atmosphere. On completion, the reaction mixture was filtered and the filtrate was concentrated under reduced pressure to give a residue. The residue was purified by column chromatography (SiO₂, Petroleum ether/Ethyl acetate=1/0 to 12/1) to give the title compound (250 mg, 4% yield) as a yellow solid. LC-MS (ESI⁺) m/z 370.9 (M+1)⁺.

Step 2—Tert-butyl 4-(2,6-dimethyl-4-(4,4,5,5-tetramethyl-1,3,2-dioxaborolan-2-yl)phenyl)piperazine-1-carboxylate. To a solution of tert-butyl 4-(4-bromo-2,6-dimethyl-phenyl)piperazine-1-carboxylate (250 mg, 677 umol) and 4,4,5,5-tetramethyl-2-(4,4,5,5-tetramethyl-1,3,2-dioxaborolan-2-yl)-1,3,2-dioxaborolane (429 mg, 1.69 mmol) in dioxane (5 mL) was added KOAc (199 mg, 2.03 mmol) and Pd(dppf)Cl₂ (49.5 mg, 67.7 umol). Then the mixture was stirred at 100° C. for 12 hrs under N₂ atmosphere. The reaction mixture was filtered and the filtrate was concentrated under reduced pressure to give a residue. The residue was purified by column chromatography (SiO₂, Petroleum ether/Ethyl acetate=1/0 to 10/1) to give the title compound (260 mg, 92% yield) as a brown solid. LC-MS (ESI⁺) m/z 417. (M+1)⁺.

### 2′-(2-(3,5-Dimethyl-4-(piperazin-1-yl)phenyl)pyridin-4-yl)-5′,6′-dihydrospiro[cyclopropane-1,7′-pyrrolo[3,2-c]pyridin]-4′(1′H)-one (Intermediate FY)

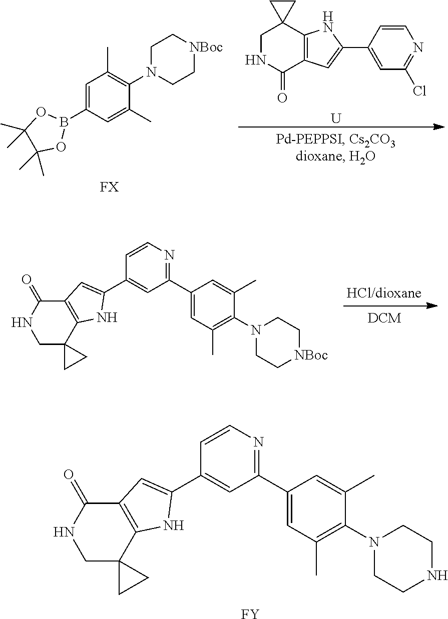

Step 1—Tert-butyl 4-(2,6-dimethyl-4-(4-(4′-oxo-1′,4′,5′,6′-tetrahydrospiro[cyclopropane-1,7′-pyrrolo[3,2-c]pyridin]-2′-yl)pyridin-2-yl)phenyl)piperazine-1-carboxylate. To a solution of tert-butyl 4-[2,6-dimethyl-4-(4,4,5,5-tetramethyl-1,3,2-dioxaborolan-2-yl)phenyl] piperazine-1-carboxylate (219 mg, 526 umol, Intermediate FX) and 2-(2-chloro-4-pyridyl)spiro[5,6-dihydro-1H-pyrrolo[3,2-c]pyridine-7,1′-cyclopropane]-4-one (120 mg, 438 umol, Intermediate U) in dioxane (5 mL) and H₂O (1 mL) was added Cs₂CO₃ (428 mg, 1.32 mmol) and 1,3-bis[2,6-bis(1-propylbutyl)phenyl]-4,5-dichloro-2H-imidazol-1-ium-2-ide;3-chloropyridine dichloropalladium (42.7 mg, 43.8 umol, CAS #1814936-54-3). The mixture was stirred at 80° C. or 2 hrs under N₂ atmosphere. The reaction mixture was diluted with H₂O (10 mL) and extracted with EA (10 mL×3). The combined organic layers were dried over Na₂SO₄, filtered and the filtrate was concentrated under reduced pressure to give the title compound (300 mg) as a brown solid. LC-MS (ESI⁺) m/z 528.2 (M+1)⁺.

Step 2—2′-(2-(3,5-dimethyl-4-(piperazin-1-yl)phenyl)pyridin-4-yl)-5′,6′-dihydrospiro[cyclopropane-1,7-pyrrolo[3,2-c]pyridin]-4′(1′H)-one. To a solution of tert-butyl 4-[2,6-dimethyl-4-[4-(4-oxospiro[5,6-dihydro-1H-pyrrolo[3,2-c]pyridine-7,1′-cyclopropane]-2-yl)-2-pyridyl]phenyl]piperazine-1-carboxylate (150 mg, 284 umol) in DCM (10 mL) was added HCl/dioxane (4 M, 2 mL), the mixture was stirred at 25° C. for 0.5 hrs. The reaction mixture was diluted with DCM (20 mL), then filtered and the filter cake was concentrated under reduced pressure to give the title compound (155 mg, HCl) as a brown solid. LC-MS (ESI⁺) m/z 428.2 (M+1)⁺.

### Tert-butyl 4-(3-(1H-indol-6-yl)-5-(4,4,5,5-tetramethyl-1,3,2-dioxaborolan-2-yl)phenyl)piperazine-1-carboxylate (Intermediate FZ)

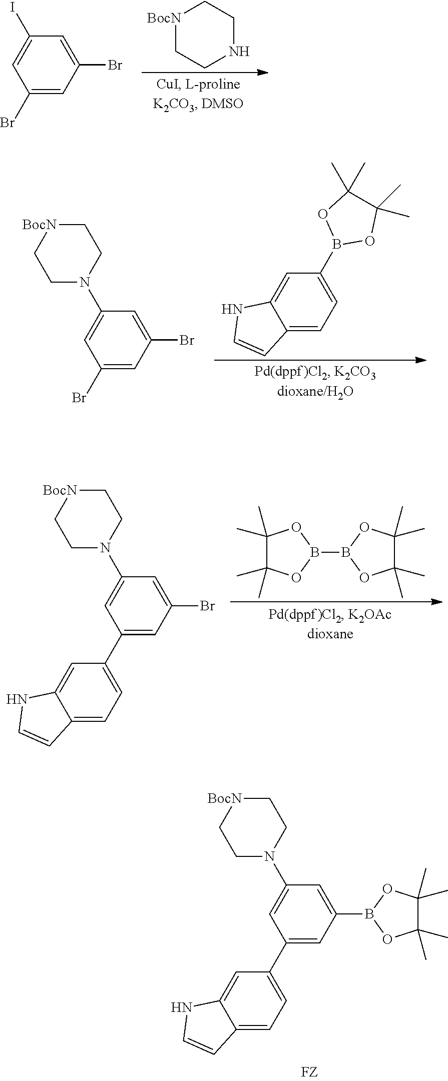

Step 1—Tert-butyl 4-(3,5-dibromophenyl)piperazine-1-carboxylate. To a solution of 1,3-dibromo-5-iodo-benzene (6.6 g, 18.2 mmol) in DMSO (60 mL) was added tert-butyl piperazine-1-carboxylate (3.40 g, 18.2 mmol), CuI (695 mg, 3.65 mmol), L-proline (840 mg, 7.30 mmol) and K₂CO₃ (5.04 g, 36.5 mmol), and purged with N₂ three times. Then the mixture was stirred at 80° C. for 12 hrs under N₂ atmosphere. On completion, the reaction mixture was partitioned between H₂O (150 mL) and ethyl acetate (250 mL). The organic phase was separated, washed with brine (70 mL×3), dried over Na₂SO₄, filtered and concentrated under reduced pressure to give a residue. The residue was purified by column chromatography (SiO₂, Petroleum ether/Ethyl acetate=1/0 to 20/1) to give the title compound (2.7 g, 35% yield) as a yellow solid. LC-MS (ESI+) m/z 412.8 (M+H)⁺.

Step 2—Tert-butyl 4-(3-bromo-5-(1H-indol-6-yl)phenyl)piperazine-1-carboxylate. To a solution of tert-butyl 4-(3,5-dibromophenyl)piperazine-1-carboxylate (2.5 g, 5.95 mmol) in dioxane (30 mL) and H₂O (8 mL) was added 6-(4,4,5,5-tetramethyl-1,3,2-dioxaborolan-2-yl)-1H-indole (1.30 g, 5.36 mmol), Pd(dppf)Cl₂ (435 mg, 595 umol) and K₂CO₃ (2.47 g, 17.85 mmol) and the mixture was purged with N₂ three times. Then the mixture was stirred at 80° C. for 2 hrs under N₂ atmosphere. On completion, the reaction mixture was partitioned between H₂O (90 mL) and Ethyl acetate (120 mL). The organic phase was separated, washed with brine (60 mL×3), dried over Na₂SO₄, filtered and concentrated under reduced pressure to give a residue. The residue was purified by column chromatography (SiO₂, Petroleum ether/Ethyl acetate=20/1 to 5/1) to give the tittle compound (1.4 g, 49% yield) as a white solid. LC-MS (ESI+) m/z 457.9 (M+H)⁺.

Step 3—Tert-butyl 4-(3-(1H-indol-6-yl)-5-(4,4,5,5-tetramethyl-1,3,2-dioxaborolan-2-yl)phenyl)piperazine-1-carboxylate. To a solution of tert-butyl 4-[3-bromo-5-(1H-indol-6-yl)phenyl]piperazine-1-carboxylate (1.8 g, 3.94 mmol) in dioxane (60 mL) was added 4,4,5,5-tetramethyl-2-(4,4,5,5-tetramethyl-1,3,2-dioxaborolan-2-yl)-1,3,2-dioxaborolane (2.00 g, 7.89 mmol), Pd(dppf)Cl₂ (289 mg, 394 umol) and KOAc (1.16 g, 11.8 mmol) then the mixture was purged with N₂ three times. Then the mixture was stirred at 80° C. for 2 hrs under N₂ atmosphere. On completion, the reaction mixture was filtered and concentrated under reduced pressure to give a residue. The residue was purified by column chromatography (SiO₂, Petroleum ether/Ethyl acetate=10/1 to 5/1) to give the tittle compound (1.4 g, 67% yield) as a white solid. LC-MS (ESI+) m/z 504.3 (M+H)⁺.

### 2-(2-(3-(1H-indol-6-yl)-5-(piperazin-1-yl)phenyl)pyridin-4-yl)-6,7-dihydro-1H-pyrrolo[3,2-c]pyridin-4(5H)-one (Intermediate GA)

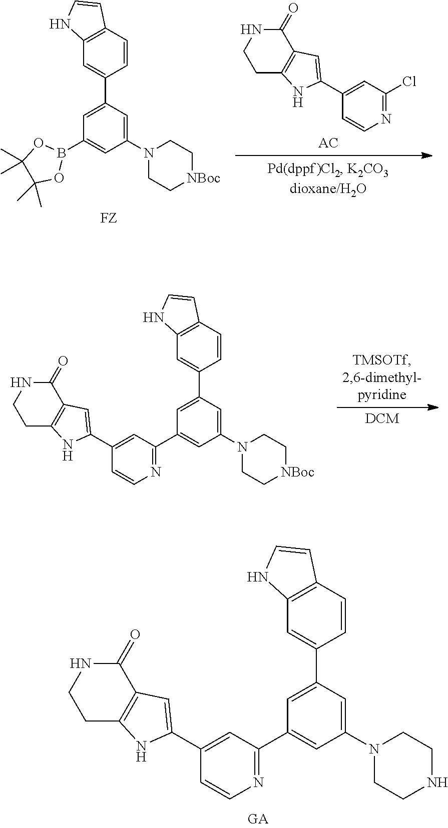 [SMILES: CC(C)(C)OC(=O)N1CCN(c2cc(B3OC(C)(C)C(C)(C)O3)cc(-c3ccc4cc[nH]c4c3)c2)CC1 ; O=C1NCCc2[nH]c(-c3ccnc(Cl)c3)cc21 ; CC(C)(C)OC(=O)N1CCN(c2cc(-c3ccc4cc[nH]c4c3)cc(-c3cc(-c4cc5c([nH]4)CCNC5=O)ccn3)c2)CC1 ; O=C1NCCc2[nH]c(-c3ccnc(-c4cc(-c5ccc6cc[nH]c6c5)cc(N5CCNCC5)c4)c3)cc21]

Step 1—Tert-butyl 4-(3-(1H-indol-6-yl)-5-(4-(4-oxo-4,5,6,7-tetrahydro-1H-pyrrolo[3,2-c]pyridin-2-yl)pyridin-2-yl)phenyl)piperazine-1-carboxylate. To a solution of 2-(2-chloro-4-pyridyl)-1,5,6,7-tetrahydropyrrolo[3,2-c]pyridin-4-one (59.0 mg, 238 umol, Intermediate AC) in dioxane (6 mL) and H₂O (1.5 mL) was added tert-butyl 4-[3-(1H-indol-6-yl)-5-(4,4,5,5-tetramethyl-1,3,2-dioxaborolan-2-yl)phenyl]piperazine-1-carboxylate (120 mg, 238 umol, Intermediate FZ), Pd(dppf)Cl₂ (17.4 mg, 23.8 umol), and K₂CO₃ (98.8 mg, 715 umol), then the mixture was degassed and purged with N₂ three times. The mixture was stirred at 80° C. for 2 hrs under N₂ atmosphere. On completion, the reaction mixture was partitioned between H₂O (20 mL) and ethyl acetate (50 mL). The organic phase was separated, washed with brine (90 mL), dried over Na₂SO₄, filtered and the filtrate was concentrated under reduced pressure to give the title compound (120 mg) as a brown solid. LC-MS (ESI+) m/z 589.5 (M+H)⁺.

Step 2—2-(2-(3-(1H-indol-6-yl)-5-(piperazin-1-yl)phenyl)pyridin-4-yl)-6,7-dihydro-1H-pyrrolo[3,2-c]pyridin-4(5H)-one. To a solution of tert-butyl 4-[3-(1H-indol-6-yl)-5-[4-(4-oxo-1,5,6,7-tetrahydropyrrolo[3,2-c]pyridin-2-yl)-2-pyridyl]phenyl]piperazine-1-carboxylate (120 mg, 204 umol) in DCM (2 mL) was added 2,6-dimethylpyridine (109 mg, 1.02 mmol) and TMSOTf (136 mg, 612 umol). The mixture was stirred at 0° C. for 1 hr. On completion, the reaction mixture was filtered and the filtrate was concentrated under reduced pressure to give a residue. The residue was purified by reversed-phase HPLC (0.1% NH₄HCO₃) to give the title compound (30 mg, 29% yield) as a brown solid. LC-MS (ESI+) m/z 489.1 (M+H)⁺.

### Example 1 (Method 1): Synthesis of 3-(5-(3-((4-(3-fluoro-4-(4-(4′-oxo-1′,4′,5′,6′-tetrahydrospiro[cyclopropane-1,7′-pyrrolo[3,2-c]pyridin]-2′-yl)pyrimidin-2-yl)phenyl)piperazin-1-yl)methyl)azetidin-1-yl)-3-methyl-2-oxo-2,3-dihydro-1H-benzo[d]imidazol-1-yl)piperidine-2,6-dionee (I-403)

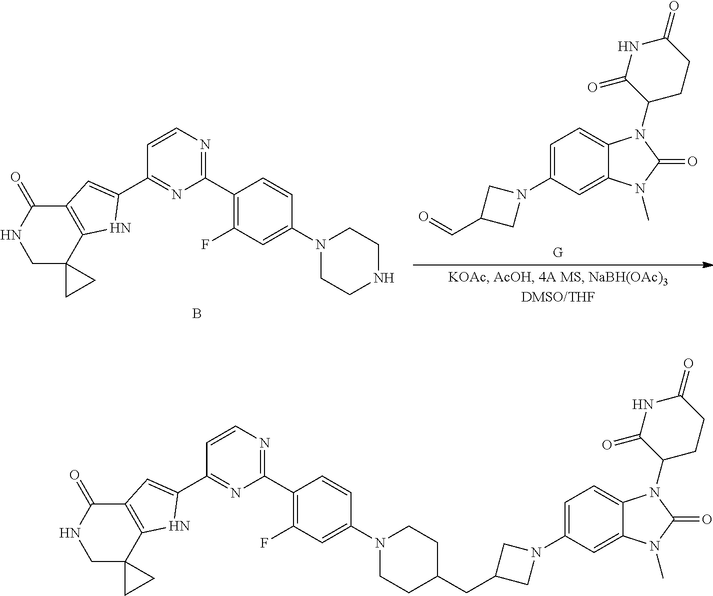

To a solution of 2-[2-(2-fluoro-4-piperazin-1-yl-phenyl)pyrimidin-4-yl]spiro[5,6-dihydro-1H-pyrrolo[3,2-c]pyridine-7,1′-cyclopropane]-4-one (250 mg, 549 umol, HCl, Intermediate B) and 1-[1-(2,6-dioxo-3-piperidyl)-3-methyl-2-oxo-benzimidazol-5-yl]azetidine-3-carbaldehyde (188 mg, 484 umol, FA, Intermediate G) in DMSO (3 mL) and THF (3 mL) was added AcOH (33.0 mg, 549 umol, 31.4 uL) and 4 Å molecular sieves (200 mg). The mixture was stirred at 25° C. for 0.5 hr, then KOAc (323 mg, 3.30 mmol) was added and the mixture was stirred at 25° C. for 0.5 hr. Finally NaBH(OAc)₃ (349 mg, 1.65 mmol) was added and the mixture was stirred at 25° C. for 1.5 hr. On completion, the reaction mixture was filtered to remove the insoluble and concentrated under reduced pressure to give a residue. The residue was purified by reversed-phase HPLC (neutral condition) to give the title compound (100 mg, 24% yield) as a white solid. ¹H NMR (400 MHz, DMSO-d₆) δ=11.41 (s, 1H), 11.17-10.92 (m, 1H), 8.73-8.63 (m, 1H), 8.20-8.09 (m, 1H), 7.63-7.55 (m, 1H), 7.29-7.24 (m, 1H), 7.22-7.17 (m, 1H), 6.93-6.86 (m, 2H), 6.83-6.75 (m, 1H), 6.36-6.27 (m, 1H), 6.15-6.05 (m, 1H), 5.31-5.21 (m, 1H), 3.94 (t, J=7.4 Hz, 2H), 3.45 (br t, J=6.4 Hz, 2H), 3.28 (s, 3H), 3.26 (br d, J=2.0 Hz, 2H), 3.01-2.87 (m, 2H), 2.63 (br d, J=7.2 Hz, 4H), 2.60-2.52 (m, 8H), 2.02-1.92 (m, 1H), 1.38-1.32 (m, 2H), 1.01-0.96 (m, 2H) LC-MS (ESI⁺) m/z 745.2 (M+H)⁺.

TABLE 2
Compounds synthesized via Method 1 using the corresponding
amines and aldehyde/ketones for the reductive amination.

| | | | LCMS (ESI+) | |
|---|---|---|---|---|
| I-#^{a} | Amine | Aldehyde/ketone | m/z (M + H)⁺ | ¹H NMR (400 MHz, DMSO-d6) |
| I-93 | CL | CC | 743.2 | 11.09 (s, 1H), 9.22 (d, J = 8.8 Hz, 1H), 8.28 (s, 1H), 8.22 (d, |
| | | | | J = 8.8 Hz, 1H), 8.15-8.11 (m, 1H), 8.08 (d, J = 4.4 Hz, 1H), |
| | | | | 8.03 (d, J = 8.8 Hz, 1H), 7.86 (s, 1H), 7.69 (d, J = 7.6 Hz, |
| | | | | 1H), 7.41 (t, J = 8.0 Hz, 1H), 7.16 (t, J = 5.2 Hz, 1H), 7.12- |
| | | | | 7.07 (m, 1H), 7.00-6.96 (m, 2H), 6.95-6.91 (m, 1H), 5.40- |
| | | | | 5.33 (m, 1H), 3.48 (s, 3H), 3.27 (s, 3H), 2.98 (t, J = 7.6 Hz, |
| | | | | 2H), 2.91-2.83 (m, 1H), 2.73-2.67 (m, 1H), 2.62 (d, J = 4.4 |
| | | | | Hz, 1H), 2.59 (s, 4H), 2.55-2.53 (m, 3H), 2.46 (t, J = 6.8 Hz, |
| | | | | 2H), 2.03-1.95 (m, 1H), 1.83 (q, J = 7.2 Hz, 2H), 1.21 (d, J = |
| | | | | 6.8 Hz, 3H) |
| I-96 | FF | FH | 797.2 | 11.11 (s, 1H), 10.93 (s, 1H), 9.49 (d, J = 8.8 Hz, 1H), 8.78 (d, |
| | | | | J = 0.8 Hz, 1H), 8.41-8.31 (m, 2H), 8.29-8.22 (m, 2H), |
| | | | | 8.17 (s, 1H), 8.03 (d, J = 6.4 Hz, 1H), 7.78 (t, J = 8.0 Hz, |
| | | | | 1H), 7.13-6.95 (m, 3H), 5.46-5.38 (m, 1H), 3.78-3.74 (m, |
| | | | | 2H), 3.72-3.58 (m, 8H), 3.56-3.36 (m, 3H), 3.31-3.07 (m, |
| | | | | 4H), 2.98-2.86 (m, 1H), 2.78-2.69 (m, 1H), 2.68-2.60 (m, |
| | | | | 1H), 2.55 (s, 1H), 2.47-2.39 (m, 2H), 2.37-2.26 (m, 2H), |
| | | | | 2.13-1.92 (m, 5H), 1.21 (d, J = 6.8 Hz, 3H) |
| I-98 | FF | DU | 797.2 | 11.11 (s, 1H), 10.24-10.09 (m, 1H), 9.49-9.31 (m, 1H), |
| | | | | 8.31-8.23 (m, 4H), 8.17 (d, J = 2.8 Hz, 1H), 7.33-7.18 (m, |
| | | | | 3H), 7.12-6.99 (m, 3H), 5.47-5.35 (m, 1H), 4.04-3.96 (m, |
| | | | | 2H), 3.67-3.60 (m, 8H), 3.49 (s, 2H), 3.25-3.13 (m, 2H), |
| | | | | 3.04 (d, J = 5.2 Hz, 2H), 3.01-2.94 (m, 1H), 2.93-2.86 (m, |
| | | | | 1H), 2.78-2.69 (m, 1H), 2.66-2.60 (m, 1H), 2.55 (s, 1H), |
| | | | | 2.41-2.35 (m, 1H), 2.25-2.16 (m, 1H), 2.02 (d, J = 12.4 Hz, |
| | | | | 5H), 1.50-1.32 (m, 2H), 1.21 (d, J = 6.8 Hz, 3H) |
| I-99 | FF | EE | 798.2 | 11.09 (s, 1H), 9.16 (br d, J = 9.2 Hz, 1H), 9.05 (s, 1H), 8.43 |
| | | | | (br d, J = 8.8 Hz, 1H), 8.20-8.14 (m, 1H), 8.07 (br s, 1H), |
| | | | | 8.06 (br d, J = 2.4 Hz, 1H), 7.95 (br d, J = 9.2 Hz, 1H), 7.15- |
| | | | | 7.09 (m, 1H), 7.05-6.95 (m, 4H), 5.37 (br dd, J = 5.2, 11.9 |
| | | | | Hz, 1H), 4.45 (br d, J = 12 Hz, 2H), 3.59 (s, 3H), 3.47 (br s, |
| | | | | 2H), 3.24-3.23 (m, 1H), 3.01-2.96 (m, 3H), 2.91 (br d, J = |
| | | | | 12 Hz, 2H), 2.72 (br d, J = 2.8 Hz, 1H), 2.67 (br d, J = 2.0 |
| | | | | Hz, 1H), 2.59 (br d, J = 2.0 Hz, 2H), 2.22 (br d, J = 6.4 Hz, |
| | | | | 2H), 2.11-2.05 (m, 2H), 2.02-1.98 (m, 1H), 1.87-1.78 (m, |
| | | | | 6H), 1.20 (br d, J = 6.4 Hz, 3H), 1.16-1.10(m, 2H) |
| I-107 | FM | FH | 826.3 | 12.82-12.22 (m, 1H), 12.14-11.39 (m, 1H), 11.15-11.08 |
| | | | | (m, 1H), 9.40 (br d, J = 8.8 Hz, 1H), 8.33 (d, J = 8.8 Hz, 1H), |
| | | | | 8.25-8.22 (m, 1H), 8.15 (br d, J = 8.8 Hz, 2H), 7.85-7.64 |
| | | | | (m, 2H), 7.09-7.00 (m, 3H), 5.41 (br dd, J = 5.4, 12.6 Hz, |
| | | | | 1H), 3.93 (br d, J = 2.0 Hz, 2H), 3.87-3.78 (m, 5H), 3.66 (s, |
| | | | | 4H), 3.50 (br s, 7H), 3.33-3.17 (m, 2H), 2.97-2.85 (m, 1H), |
| | | | | 2.78-2.69 (m, 1H), 2.66 (br s, 1H), 2.61 (br s, 2H), 2.55 (br |
| | | | | s, 1H), 2.53 (br d, J = 2.0 Hz, 3H), 2.28-2.20 (m, 2H), 2.03- |
| | | | | 1.97 (m, 1H), 1.93-1.78 (m, 2H), 1.21 (d, J = 6.6 Hz, 3H) |
| I-118 | CL | DO | 743.4 | 11.09 (s, 1H), 9.22 (d, J = 9.2 Hz, 1H), 8.22 (d, J = 8.8 Hz, |
| | | | | 1H), 8.13 (d, J = 8.8 Hz, 1H), 8.09 (d, J = 4.0 Hz, 1H), 8.03 |
| | | | | (d, J = 8.8 Hz, 1H), 7.86 (s, 1H), 7.69 (d, J = 7.6 Hz, 1H), |
| | | | | 7.41 (t, J = 8.0 Hz, 1H), 7.16 (t, J = 5.2 Hz, 1H), 7.12-7.07 |
| | | | | (m, 2H), 7.02 (d, J = 8.0 Hz, 1H), 6.91 (d, J = 8.0 Hz, 1H), |
| | | | | 5.41-5.27 (m, 1H), 3.65-3.59 (m, 1H), 3.48 (s, 2H), 3.34 (s, |
| | | | | 3H), 3.30 (d, J = 4.0 Hz, 4H), 2.95-2.86 (m, 1H), 2.77-2.72 |
| | | | | (m, 1H), 2.68 (t, J = 7.6 Hz, 3H), 2.58 (s, 4H), 2.39 (t, J = 7.2 |
| | | | | Hz, 2H), 2.05-1.97 (m, 1H), 1.88-1.79 (m, 2H), 1.21 (d, J = |
| | | | | 6.8 Hz, 3H) |
| I-125 | J | AF | 799.4 | 11.09 (s, 1H), 9.16 (d, J = 9.1 Hz, 1H), 9.06 (s, 1H), 8.46 (d, J = |
| | | | | 8.5 Hz, 1H), 8.17 (d, J = 8.6 Hz, 1H), 8.12-8.02 (m, 2H), |
| | | | | 7.96 (d, J = 9.2 Hz, 1H), 7.12 (s, 1H), 7.05-6.95 (m, 2H), |
| | | | | 6.89 (dd, J = 7.2, 14.8 Hz, 2H), 5.41-5.28 (m, 1H), 3.64 (s, |
| | | | | 8H), 3.47 (s, 3H), 3.13 (d, J = 7.6 Hz, 2H), 2.89 (s, 1H), 2.79- |
| | | | | 2.55 (m, 6H), 2.33 (s, 1H), 2.27 (d, J = 3.6 Hz, 2H), 1.99 |
| | | | | (dd, J = 4.0, 5.6 Hz, 1H), 1.87 (d, J = 10.4 Hz, 2H), 1.77- |
| | | | | 1.64 (m, 1H), 1.42-1.28 (m, 2H), 1.19 (d, J = 6.0 Hz, 3H) |
| I-126 | AQ | AF | 798.3 | 11.08 (s, 1H), 9.15 (d, J = 9.2 Hz, 1H), 8.19 (br d, J = 8.8 Hz, |
| | | | | 2H), 8.13 (d, J = 9.2 Hz, 1H), 8.10-8.03 (m, 2H), 7.96 (d, J = |
| | | | | 8.8 Hz, 1H), 7.09 (br d, J = 8.8 Hz, 3H), 7.01-6.95 (m, |
| | | | | 1H), 6.88 (br dd, J = 8.0, 15.4 Hz, 2H), 5.35 (br dd, J = 5.2, |
| | | | | 12.4 Hz, 1H), 3.64 (s, 3H), 3.47 (br s, 3H), 3.28-3.25 (m, |
| | | | | 4H), 3.13 (br d, J = 9.6 Hz, 3H), 2.93-2.84 (m, 2H), 2.76- |
| | | | | 2.69 (m, 3H), 2.64 (br s, 3H), 2.28 (br d, J = 6.0 Hz, 2H), |
| | | | | 2.00 (br dd, J = 4.8, 10.9 Hz, 1H), 1.86 (br d, J = 12.8 Hz, |
| | | | | 2H), 1.74-1.64 (m, 1H), 1.35 (dt, J = 9.6, 12.9 Hz, 2H), 1.20 |
| | | | | (d, J = 6.8 Hz, 3H) |
| I-128 | BW | FJ | 812.3 | 11.16 (s, 1H), 10.90-10.76 (m, 1H), 9.60-9.47 (m, 1H), |
| | | | | 8.40 (d, J = 0.8 Hz, 2H), 8.36-8.31 (m, 2H), 8.25 (d, J = |
| | | | | 1.6 Hz, 1H), 7.45-7.29 (m, 2H), 7.13-7.08 (m, 1H), 7.06- |
| | | | | 6.97 (m, 2H), 5.44 (J = 5.2, 12.6 Hz, 1H), 4.07-4.00 (m, |
| | | | | 4H), 3.71 (s, 3H), 3.66-3.62 (m, 2H), 3.55 (s, 2H), 3.32 (s, |
| | | | | 8H), 3.21-3.04 (m, 2H), 3.01-2.91 (m, 1H), 2.79-2.69 (m, |
| | | | | 2H), 2.09-2.03 (m, 1H), 1.97-1.89 (m, 2H), 1.86-1.72 (m, |
| | | | | 3H), 1.57-1.37 (m, 2H), 1.30-1.23 (m, 3H) |
| I-129 | J | W | 799.3 | 11.12 (s, 1H), 9.22-9.13 (m, 1H), 9.09-9.02 (m, 1H), 8.49- |
| | | | | 8.43 (m, 1H), 8.17 (br d, J = 9.2 Hz, 1H), 8.13-8.04 (m, 2H), |
| | | | | 7.99-7.94 (m, 1H), 7.16-7.08 (m, 1H), 7.01 (br d, J = 8.8 |
| | | | | Hz, 1H), 6.94 (br d, J = 7.2 Hz, 1H), 6.84 (br s, 1H), 6.65 (br |
| | | | | d, J = 8.8 Hz, 1H), 5.35-5.24 (m, 1H), 3.64 (br s, 7H), 3.47 |
| | | | | (br s, 3H), 2.94-2.85 (m, 2H), 2.70-2.63 (m, 5H), 2.33 (br |
| | | | | s, 2H), 2.25 (br d, J = 5.6 Hz, 2H), 2.04-1.95 (m, 2H), 1.88- |
| | | | | 1.82 (m, 2H), 1.77-1.65 (m, 2H), 1.31-1.24 (m, 2H), 1.20 |
| | | | | (br d, J = 6.0 Hz, 3H) |
| I-131 | DF | FJ | 812.3 | 11.23-11.12 (m, 1H), 11.07 (s, 1H), 9.57 (d, J = 8.8 Hz, 1H), |
| | | | | 8.49 (d, J = 2.4 Hz, 1H), 8.42-8.36 (m, 2H), 8.33 (d, J = 8.4 |
| | | | | Hz, 2H), 8.23 (d, J = 1.2 Hz, 1H), 7.53-7.32 (m, 2H), 7.02 |
| | | | | (d, J = 8.8 Hz, 1H), 6.95 (d, J = 1.6 Hz, 1H), 6.76-6.69 (m, |
| | | | | 1H), 5.33 (dd, J = 5.2, 12.8 Hz, 1H), 3.97 (d, J = 8.8 Hz, |
| | | | | 2H), 3.75 (d, J = 10.8 Hz, 2H), 3.66-3.56 (m, 3H), 3.49 (s, |
| | | | | 2H), 3.33 (s, 3H), 3.26 (s, 1H), 3.24-3.14 (m, 6H), 2.97- |
| | | | | 2.85 (m, 1H), 2.77-2.67 (m, 1H), 2.66-2.58 (m, 1H), 2.55 |
| | | | | (s, 1H), 2.04-1.96 (m, 1H), 1.88 (d, J = 10.8 Hz, 2H), 1.83- |
| | | | | 1.68 (m, 3H), 1.59-1.34 (m, 2H), 1.21 (d, J = 6.8 Hz, 3H) |
| I-137 | FF | FE | 745.6 | 11.97 (br s, 1H), 11.09 (br s, 1H), 8.52 (d, J = 5.2 Hz, 1H), |
| | | | | 7.89-7.80 (m, 2H), 7.50 (td, J = 5.2 Hz, 1H), 7.05-7.01 (m, |
| | | | | 5H), 6.97 (dd, J = 2.4 Hz, 1H), 5.39-5.35 (m, 1H), 3.86 (br t, |
| | | | | J = 13.6 Hz, 2H), 3.56 (s, 3H), 2.97 (s, 2H), 2.87-2.81 (m, |
| | | | | 5H), 2.21 (d, J = 6.0 Hz, 1H), 2.07(s, 1H), 1.84-1.81 (m, |
| | | | | 3H), 1.23-1.93 (m, 7H), 1.45 (s, 3H) |
| I-179 | AT | X | 703.3 | 11.19 (br d, J = 1.6 Hz, 1H), 11.10 (s, 1H), 8.54 (d, J = 5.2 |
| | | | | Hz, 1H), 7.91 (s, 1H), 7.80 (t, J = 9.2 Hz, 1H), 7.54 (dd, J = |
| | | | | 1.6, 5.2 Hz, 1H), 7.13 (s, 1H), 7.01-6.96 (m, 3H), 6.96-6.92 |
| | | | | (m, 1H), 6.92-6.89 (m, 1H), 6.85 (br d, J = 2.0 Hz, 1H), 5.38 |
| | | | | (dd, J = 5.2, 12.4 Hz, 1H), 3.62 (s, 3H), 3.30 (br s, 2H), 3.27 |
| | | | | (d, J = 2.4 Hz, 2H), 3.16-3.10 (m, 2H), 2.93-2.85 (m, 1H), |
| | | | | 2.73 (br s, 1H), 2.66 (br d, J = 4.8 Hz, 6H), 2.61 (br d, J = 2.4 |
| | | | | Hz, 1H), 2.55 (br s, 2H), 2.06-1.97 (m, 1H), 1.30-1.25 (m, |
| | | | | 2H), 1.02-0.95 (m, 2H) |
| I-180 | BU | W | 845.4 | 11.06 (s, 1H), 9.21 (d, J = 9.2 Hz, 1H), 8.18-8.12 (m, 2H), |
| | | | | 8.08 (d, J = 3.6 Hz, 1H), 8.02 (d, J = 8.4 Hz, 2H), 7.74 (d, J = |
| | | | | 7.6 Hz, 1H), 7.14 (s, 1H), 6.94 (d, J = 8.4 Hz, 1H), 6.88-6.83 |
| | | | | (m, 2H), 6.65 (d, J = 8.4 Hz, 1H), 5.29 (dd, J = 5.2, 12.8 Hz, |
| | | | | 1H), 3.91 (s, 3H), 3.60 (d, J = 11.6 Hz, 4H), 3.51-3.44 (m, |
| | | | | 3H), 3.14 (s, 4H), 2.94-2.86 (m, 1H), 2.66 (d, J = 12.0 Hz, |
| | | | | 4H), 2.55 (s, 2H), 2.26 (d, J = 6.4 Hz, 2H), 2.03-1.96 (m, |
| | | | | 1H), 1.84 (d, J = 11.6 Hz, 2H), 1.70 (d, J = 9.2 Hz, 1H), 1.29 |
| | | | | (d, J = 11.2 Hz, 2H), 1.20 (d, J = 6.4 Hz, 3H) |
| I-181 | BW | BV | 784.4 | 11.10 (s, 1H), 8.86 (br d, J = 9.6 Hz, 1H), 7.98 (br d, J = 4.0 |
| | | | | Hz, 1H), 7.86 (d, J = 8.8 Hz, 1H), 7.58 (d, J = 9.2 Hz, 1H), |
| | | | | 7.27 (d, J = 9.6 Hz, 1H), 6.89-7.00 (m, 4H), 5.36 (br dd, J = |
| | | | | 12.8, 5.6 Hz, 1H), 3.71 (br s, 2H), 3.63 (s, 5H), 3.57 (br dd, J = |
| | | | | 7.2, 3.6 Hz, 1H), 3.42 (br s, 2H), 3.31 (s, 3H), 2.86 (br dd, J = |
| | | | | 14.4, 8.0 Hz, 5H), 2.64-2.73 (m, 3H), 2.02-2.09 (m, 4H), |
| | | | | 1.67 (br d, J = 7.6 Hz, 4H), 1.57-1.61 (m, 2H), 1.18 (d, J = |
| | | | | 6.8 Hz, 3H) |
| I-183 | J | BX | 704.3 | 10.80 (s, 1H), 9.17 (br d, J = 8.8 Hz, 1H), 9.06 (d, J = 1.6 |
| | | | | Hz, 1H), 8.46 (br d, J = 9.2 Hz, 1H), 8.18 (br d, J = 8.8 Hz, |
| | | | | 1H), 8.05-8.12 (m, 2H), 7.96 (d, J = 9.2 Hz, 1H), 7.13 (br d, |
| | | | | J = 8.4 Hz, 3H), 7.00 (br d, J = 8.8 Hz, 1H), 6.91 (br d, J = |
| | | | | 8.4 Hz, 2H), 4.01 (br t, J = 6.2 Hz, 2H), 3.79 (br dd, J = 11.6, |
| | | | | 5.32 Hz, 1H), 3.63 (br s, 6H), 3.47 (br s, 2H), 2.60-2.70 (m, |
| | | | | 3H), 2.38-2.42 (m, 2H), 2.13-2.19 (m, 1H), 1.96-2.08 (m, |
| | | | | 2H), 1.74-1.80 (m, 2H), 1.61-1.67 (m, 2H), 1.20 (br d, J = |
| | | | | 6.4 Hz, 3H) |
| I-184 | AL | BY | 798.6 | 8.92-8.85 (m, 1H), 7.99 (d, J = 3.2 Hz, 1H), 7.89 (d, J = 8.8 |
| | | | | Hz, 1H), 7.61 (d, J = 8.8 Hz, 1H), 7.55 (d, J = 8.8 Hz, 2H), |
| | | | | 7.42 (s, 1H), 7.31-7.23 (m, 2H), 7.14 (d, J = 8.4 Hz, 1H), |
| | | | | 7.02 (d, J = 8.8 Hz, 2H), 6.94 (s, 1H), 5.39 (d, J = 8.0 Hz, |
| | | | | 1H), 3.76 (d, J = 12.0 Hz, 2H), 3.70 (s, 4H), 3.60-3.56 (m, |
| | | | | 1H), 3.51 (s, 2H), 3.40-3.40 (m, 3H), 3.30-3.14 (m, 4H), |
| | | | | 2.97-2.87 (m, 1H), 2.74 (d, J = 10.8 Hz, 2H), 2.70-2.66 |
| | | | | (m, 1H), 2.29-2.19 (m, 2H), 2.07-2.01 (m, 1H), 1.86 (d, J = |
| | | | | 12.4 Hz, 2H), 1.81-1.73 (m, 1H), 1.34-1.20 (m, 4H), 1.18 |
| | | | | (d, J = 6.8 Hz, 3H) |
| I-186 | CA | W | 822.8 | 11.06 (s, 1H), 9.22 (d, J = 9.2 Hz, 1H), 8.28 (d, J = 9.2 Hz, |
| | | | | 1H), 8.16-8.12 (m, 1H), 8.09 (d, J = 4.0 Hz, 1H), 8.04 (d, J = |
| | | | | 8.8 Hz, 1H), 7.90 (s, 1H), 7.79 (s, 1H), 7.17 (t, J = 5.2 Hz, |
| | | | | 1H), 7.13 (s, 1H), 6.93 (d, J = 8.8 Hz, 1H), 6.83 (d, J = 2.0 |
| | | | | Hz, 1H), 6.64 (dd, J = 2.0, 8.8 Hz, 1H), 5.29 (dd, J = 5.6, 12.8 |
| | | | | Hz, 1H), 4.21 (s, 1H), 3.60 (d, J = 12.0 Hz, 3H), 3.48 (s, 2H), |
| | | | | 3.31 (s, 3H), 3.30-3.30 (m, 1H), 2.94-2.81 (m, 1H), 2.64 (d, |
| | | | | J = 9.2 Hz, 2H), 2.55 (d, J = 8.4 Hz, 5H), 2.52 (s, 2H), 2.26 |
| | | | | (d, J = 7.2 Hz, 2H), 2.01-1.96 (m, 1H), 1.84 (d, J = 11.2 Hz, |
| | | | | 2H), 1.74-1.66 (m, 1H), 1.34-1.26 (m, 2H), 1.23 (s, 1H), |
| | | | | 1.20 (d, J = 6.8 Hz, 3H) |
| I-188^{b} | V | CB | 708.3 | 11.13 (br d, J = 1.2 Hz, 1H), 10.50 (s, 1H), 9.29 (s, 1H), 8.47 |
| | | | | (d, J = 5.2 Hz, 1H), 8.44 (s, 1H), 8.09-8.03 (m, 3H), 7.60 (t, |
| | | | | J = 8.0 Hz, 1H), 7.48 (dd, J = 1.6, 5.2 Hz, 1H), 7.25 (d, J = |
| | | | | 8.4 Hz, 1H), 7.12 (d, J = 2.4 Hz, 2H), 7.06 (br d, J = 9.2 Hz, |
| | | | | 2H), 6.61 (d, J = 7.6 Hz, 1H), 4.43 (td, J = 7.6, 11.1 Hz, 2H), |
| | | | | 3.98-3.92 (m, 2H), 3.85 (ddd, J = 5.2, 9.6, 12.4 Hz, 2H), |
| | | | | 3.73-3.62 (m, 2H), 3.27 (br d, J = 2.4 Hz, 2H), 3.14-3.07 |
| | | | | (m, 1H), 2.99-2.89 (m, 2H), 2.75 (td, J = 5.6, 16.8 Hz, 4H), |
| | | | | 2.63 (br d, J = 4.8 Hz, 4H), 1.30-1.26 (m, 2H), 1.03-0.99 |
| | | | | (m, 2H) |
| I-189 | V | G | 726.5 | 11.14 (br s, 1H), 11.06 (s, 1H), 8.48 (s, 1H), 8.11-8.00 (m, |
| | | | | 3H), 7.49 (d, J = 4.0 Hz, 1H), 7.13 (d, J = 2.0 Hz, 2H), 7.05 |
| | | | | (br d, J = 8.8 Hz, 2H), 6.91 (d, J = 8.4 Hz, 1H), 6.33 (d, J = |
| | | | | 1.6 Hz, 1H), 6.12 (dd, J = 1.2, 8.4 Hz, 1H), 5.28 (br dd, J = |
| | | | | 5.2, 12.8 Hz, 1H), 3.95 (s, 2H), 3.46 (s, 2H), 3.29 (s, 3H), |
| | | | | 3.28-3.27 (m, 2H), 3.27-3.23 (m, 4H), 3.00-2.92 (m, 1H), |
| | | | | 2.91-2.83 (m, 1H), 2.73-2.61 (m, 4H), 2.56 (br s, 4H), 2.02- |
| | | | | 1.94 (m, 1H), 1.32-1.27 (m, 2H), 1.01 (s, 2H) |
| I-190 | V | FK | 726.5 | 11.10 (br d, J = 9.0 Hz, 2H), 8.47 (d, J = 5.2 Hz, 1H), 8.08 (br |
| | | | | s, 1H), 8.04 (br d, J = 8.4 Hz, 2H), 7.48 (d, J = 5.2 Hz, 1H), |
| | | | | 7.12 (br d, J = 1.6 Hz, 2H), 6.97 (t, J = 8.0 Hz, 1H), 6.74 (d, J = |
| | | | | 8.0 Hz, 1H), 6.67 (d, J = 8.0 Hz, 1H), 5.32 (br dd, J = 5.2, |
| | | | | 12.4 Hz, 1H), 3.97 (t, J = 6.8 Hz, 2H), 3.58 (br s, 3H), 3.54 (t, |
| | | | | J = 6.4 Hz, 2H), 3.26 (d, J = 6.0 Hz, 6H), 2.85-2.95 (m, 2H), |
| | | | | 2.85-2.95 (m, 2H), 2.72-2.61 (m, 3H), 2.68-2.53 (m, 5H), 2.0- |
| | | | | 1.92 (m, 1H), 1.28 (t, J = 6.4 Hz, 2H), 1.01 (t, J = 5.6 Hz, 2H) |
| I-191 | AB | CC | 727.3 | 11.45 (br s, 1H), 11.10 (s, 1H), 8.49 (d, J = 5.2 Hz, 1H), 8.15 |
| | | | | (d, J = 5.2 Hz, 2H), 8.06 (br d, J = 8.8 Hz, 2H), 7.53 (d, J = |
| | | | | 5.2 Hz, 1H), 7.15 (br s, 1H), 7.09 (d, J = 2.0 Hz, 1H), 7.05 (br |
| | | | | d, J = 8.8 Hz, 2H), 6.97 (s, 2H), 6.93 (br d, J = 3.6 Hz, 1H), |
| | | | | 5.37 (br dd, J = 12.4, 5.2 Hz, 1H), 3.60 (s, 3H), 3.25 (br s, |
| | | | | 4H), 3.18 (br d, J = 1.6 Hz, 2H), 2.84-3.00 (m, 4H), 2.61- |
| | | | | 2.73 (m, 3H), 2.59 (br s, 1H), 2.44 (br s, 1H), 1.90-2.02 (m, |
| | | | | 4H), 1.83 (br d, J = 6.4 Hz, 7H), 1.73 (br d, J = 6.8 Hz, 2H) |
| I-192 | AB | W | 782.3 | 11.50 (br d, J = 1.6 Hz, 1H), 11.12 (s, 1H), 8.55 (d, J = 5.6 |
| | | | | Hz, 1H), 8.21 (s, 2H), 8.12 (d, J = 8.8 Hz, 2H), 7.59 (dd, J = |
| | | | | 5.2, 1.6 Hz, 1H), 7.20 (br s, 1H), 7.15 (d, J = 2.4 Hz, 1H), |
| | | | | 7.11 (br d, J = 9.2 Hz, 2H), 6.99 (d, J = 8.4 Hz, 1H), 6.89 (d, |
| | | | | J = 2.0 Hz, 1H), 6.70 (dd, J = 8.4, 2.0 Hz, 1H), 5.35 (dd, J = |
| | | | | 12.4, 5.2 Hz, 1H), 3.66 (br d, J = 11.2 Hz, 2H), 3.33 (br s, |
| | | | | 2H), 3.24 (br d, J = 2.4 Hz, 2H), 2.88-3.02 (m, 2H), 2.67- |
| | | | | 2.77 (m, 8H), 2.32 (br d, J = 6.8 Hz, 2H), 1.98-2.09 (m, 4H), |
| | | | | 1.84-1.97 (m, 8H), 1.71-1.83 (m, 4H), 1.30-1.39 (m, 2H) |
| I-194 | FY | W | 782.5 | 11.14 (br s, 1H), 11.07 (s, 1H), 8.51 (d, J = 5.2 Hz, 1H), 8.10 |
| | | | | (s, 1H), 7.78 (s, 2H), 7.55 (d, J = 6.4 Hz, 1H), 7.18 (d, J = 2.4 |
| | | | | Hz, 1H), 7.13 (br s, 1H), 6.94 (d, J = 8.8 Hz, 1H), 6.84 (d, J = |
| | | | | 1.6 Hz, 1H), 6.67-6.62 (m, 1H), 5.30 (br dd, J = 5.2, 12.8 |
| | | | | Hz, 1H), 3.61 (br d, J = 11.6 Hz, 2H), 3.32 (br s, 3H), 3.28 (br |
| | | | | d, J = 2.0 Hz, 2H), 3.09 (br s, 4H), 2.93-2.84 (m, 1H), 2.70 |
| | | | | (br d, J = 4.0 Hz, 1H), 2.62 (br s, 4H), 2.52-2.52 (m, 2H), |
| | | | | 2.39 (s, 6H), 2.27 (br d, J = 7.2 Hz, 2H), 2.02-1.96 (m, 1H), |
| | | | | 1.85 (br d, J = 11.6 Hz, 2H), 1.75-1.66 (m, 1H), 1.34-1.26 |
| | | | | (m, 4H), 1.01 (t, J = 5.2 Hz, 2H) |
| I-196 | AL | AF | 722.5 | 11.09 (s, 1H), 8.89 (d, J = 9.6 Hz, 1H), 7.98 (d, J = 4.4 Hz, |
| | | | | 1H), 7.88 (d, J = 8.8 Hz, 1H), 7.61 (d, J = 9.2 Hz, 1H), 7.26 |
| | | | | (d, J = 9.6 Hz, 1H), 7.01-6.96 (m, 1H), 6.95-6.90 (m, 2H), |
| | | | | 6.87 (d, J = 7.6 Hz, 1H), 5.36 (dd, J = 5.6, 12.6 Hz, 1H), 3.70 |
| | | | | (s, 4H), 3.65 (s, 3H), 3.58 (dd, J = 2.4, 6.6 Hz, 1H), 3.43 (d, J = |
| | | | | 4.4 Hz, 3H), 3.30 (s, 1H), 3.14 (d, J = 9.6 Hz, 2H), 2.90 (s, |
| | | | | 1H), 2.73 (dd, J = 4.4, 12.5 Hz, 3H), 2.68 (td, J = 2.0, 3.6 Hz, |
| | | | | 2H), 2.65 (s, 1H), 2.29 (d, J = 4.4 Hz, 2H), 2.04-1.97 (m, |
| | | | | 1H), 1.88 (d, J = 12 Hz, 2H), 1.78-1.68 (m, 1H), 1.38 (s, |
| | | | | 2H), 1.18 (d, J = 6.8 Hz, 3H) |
| I-197^{b} | AD | CB | 682.3 | 11.96 (br s, 1H), 10.52-10.45 (m, 1H), 9.29 (s, 1H), 8.48 (d, |
| | | | | J = 5.2 Hz, 1H), 8.44 (s, 1H), 8.14 (s, 1H), 8.10 (s, 1H), 8.06 |
| | | | | (br d, J = 8.8 Hz, 2H), 7.60 (t, J = 8.0 Hz, 1H), 7.46 (d, J = |
| | | | | 5.2 Hz, 1H), 7.24 (d, J = 8.0 Hz, 1H), 7.11 (d, J = 2.0 Hz, |
| | | | | 1H), 7.05 (br d, J = 8.8 Hz, 3H), 6.61 (d, J = 7.6 Hz, 1H), |
| | | | | 4.48-4.39 (m, 2H), 3.94 (br s, 2H), 3.85 (ddd, J = 5.2, 9.6, |
| | | | | 12.0 Hz, 1H), 3.67 (td, J = 6.4, 12.0 Hz, 1H), 3.44-3.41 (m, |
| | | | | 2H), 3.30-3.29 (m, 2H), 3.14-3.06 (m, 1H), 2.98-2.85 (m, |
| | | | | 4H), 2.79-2.72 (m, 3H), 2.61 (br s, 4H) |
| I-198 | CH | CG | 710.2 | 11.97 (br s, 1H), 10.54 (s, 1H), 9.44 (s, 1H), 8.54 (s, 1H), |
| | | | | 8.47 (d, J = 52 Hz, 1H), 8.09 (s, 1H), 8.04 (br d, J = 8.4 Hz, |
| | | | | 2H), 7.76-7.70 (m, 1H), 7.63 (br d, J = 8.4 Hz, 1H), 7.45 (br |
| | | | | d, J = 5.2 Hz, 1H), 7.29 (br d, J = 7.6 Hz, 1H), 7.11 (s, 1H), |
| | | | | 7.07-7.02 (m, 3H), 3.96-3.90 (m, 1H), 3.87 (br d, J = 11.6 |
| | | | | Hz, 2H), 3.71 (td, J = 6.0, 12.1 Hz, 1H), 3.46-3.41 (m, 2H), |
| | | | | 3.15 (br s, 4H), 3.01-2.93 (m, 1H), 2.88 (br t, J = 6.8 Hz, |
| | | | | 2H), 2.82-2.74 (m, 4H), 2.71 (br s, 4H), 2.33 (br d, J = 6.0 |
| | | | | Hz, 2H), 1.87 (br d, J = 12.4 Hz, 2H), 1.83-1.75 (m, 1H), |
| | | | | 1.33-1.24 (m, 2H) |
| I-199 | CK | CC | 700.3 | 11.20-11.05 (m, 2H), 8.94 (s, 1H), 8.49 (d, J = 5.4 Hz, 1H), |
| | | | | 8.36-8.27 (m, 1H), 8.14-8.12 (m, 1H), 7.56-7.48 (m, 1H), |
| | | | | 7.19-7.10 (m, 2H), 7.01-6.91 (m, 4H), 5.37 (br dd, J = 4.8, |
| | | | | 12.4 Hz, 1H), 3.86-3.54 (m, 7H), 3.27 (br s, 4H), 2.99-2.64 |
| | | | | (m, 4H), 1.91 (br s, 1H), 1.29 (br s, 2H), 1.04-0.98 (m, 2H) |
| I-200 | CK | X | 686.5 | 11.12-11.05 (m, 2H), 8.92 (d, J = 2.4 Hz, 1H), 8.48 (d, J = |
| | | | | 5.4 Hz, 1H), 8.28 (dd, J = 2.4, 8.8 Hz, 1H), 8.14-8.08 (m, |
| | | | | 1H), 7.50 (dd, J = 1.6, 5.4 Hz, 1H), 7.17-7.10 (m, 2H), 6.99- |
| | | | | 6.92 (m, 4H), 5.40-5.34 (m, 1H), 3.65-3.59 (m, 7H), 3.27 |
| | | | | (d, J = 2.4 Hz, 2H), 3.15-3.09 (m, 1H), 3.10-3.09 (m, 1H), |
| | | | | 2.94-2.86 (m, 1H), 2.77-2.71 (m, 1H), 2.62 (br d, J = 5.2 |
| | | | | Hz, 7H), 2.05-1.97 (m, 1H), 1.31-1.26 (m, 2H), 1.05-0.99 |
| | | | | (m, 2H) |
| I-203 | V | X | 685.4 | 11.17-11.09 (m, 2H), 8.48 (d, J = 5.6 Hz, 1H), 8.19 (s, 1H), |
| | | | | 8.09 (s, 1H), 8.07-8.03 (m, 2H), 7.49 (dd, J = 1.2, 5.2 Hz, |
| | | | | 1H), 7.13 (d, J = 2.4 Hz, 2H), 7.05 (d, J = 8.8 Hz, 2H), 7.01- |
| | | | | 6.98 (m, 2H), 6.96-6.93 (m, 1H), 5.43-5.33 (m, 1H), 3.62 |
| | | | | (s, 3H), 3.28-3.27 (m, 2H), 3.16-3.10 (m, 2H), 2.96-2.85 |
| | | | | (m, 1H), 2.78-2.72 (m, 1H), 2.72-2.63 (m, 8H), 2.62 (s, |
| | | | | 1H), 2.06-1.97 (m, 1H), 1.32-1.27 (m, 2H), 1.03-0.99 (m, |
| | | | | 2H) |
| I-204 | BM | CC | 713.5 | 11.71 (br s, 1H), 11.10 (br s, 1H), 8.50 (d, J = 5.2 Hz, 1H), |
| | | | | 8.18 (s, 1H), 8.08 (d, J = 8.8 Hz, 2H), 7.55 (d, J = 5.2 Hz, |
| | | | | 1H), 7.18 (br s, 1H), 7.09 (d, J = 2.0 Hz, 1H), 7.05 (br d, J = |
| | | | | 8.8 Hz, 2H), 7.00-6.96 (m, 2H), 6.94-6.91 (m, 1H), 5.37 (br |
| | | | | dd, J = 5.6, 12.8 Hz, 1H), 3.60 (s, 3H), 3.47 (br d, J = 1.6 Hz, |
| | | | | 2H), 3.25 (br s, 4H), 2.97 (br t, J = 7.6 Hz, 2H), 2.91-2.83 |
| | | | | (m, 1H), 2.77-2.70 (m, 1H), 2.68 (br s, 1H), 2.64 (br s, 1H), |
| | | | | 2.59 (br s, 1H), 2.55 (br s, 4H), 2.44 (br t, J = 7.2 Hz, 2H), |
| | | | | 2.09 (br s, 1H), 2.07-2.02 (m, 2H), 2.01-1.95 (m, 2H), 1.86- |
| | | | | 1.79 (m, 2H) |
| I-205 | BM | W | 768.6 | 11.70 (br s, 1H), 11.06 (br s, 1H), 8.50 (d, J = 5.2 Hz, 1H), |
| | | | | 8.18 (s, 1H), 8.08 (d, J = 8.8 Hz, 2H), 7.55 (d, J = 5.2 Hz, |
| | | | | 1H), 7.17 (br s, 1H), 7.01-7.11 (m, 3H), 6.94 (d, J = 8.8 Hz, |
| | | | | 1H), 6.83 (s, 1H), 6.65 (br d, J = 9.2 Hz, 1H), 5.29 (br dd, J = |
| | | | | 12.8, 4.8 Hz, 1H), 3.60 (br d, J = 11.6 Hz, 2H), 3.47 (br s, |
| | | | | 2H), 3.31 (br s, 3H), 3.27 (br s, 4H), 2.84-2.96 (m, 1H), 2.60- |
| | | | | 2.72 (m, 5H), 2.53-2.57 (m, 5H), 2.26 (br d, J = 7.2 Hz, |
| | | | | 2H), 1.93-2.09 (m, 5H), 1.84 (br d, J = 11.8 Hz, 2H), 1.64- |
| | | | | 1.75 (m, 1H), 1.24-1.32 (m, 2H) |
| I-206 | GA | W | 843.6 | 12.01 (br s, 1H), 11.15 (br s, 1H), 11.06 (s, 1H), 8.59 (d, J = |
| | | | | 5.2 Hz, 1H), 8.26 (s, 1H), 7.88 (s, 1H), 7.75 (s, 1H), 7.69 (br |
| | | | | s, 1H), 7.65 (d, J = 8.4 Hz, 1H), 7.60 (br d, J = 5.2 Hz, 1H), |
| | | | | 7.46-7.37 (m, 2H), 7.25 (br d, J = 16.8 Hz, 2H), 7.06 (br s, |
| | | | | 1H), 6.93 (br d, J = 8.4 Hz, 1H), 6.84 (s, 1H), 6.65 (br d, J = |
| | | | | 8.4 Hz, 1H), 6.47 (br s, 1H), 5.29 (br dd, J = 5.2, 12.8 Hz, |
| | | | | 1H), 3.61 (br d, J = 12.0 Hz, 2H), 3.46-3.41 (m, 2H), 3.37- |
| | | | | 3.35 (m, 2H), 3.32-3.30 (m, 3H), 2.89 (br t, J = 6.4 Hz, 2H), |
| | | | | 2.66 (br s, 2H), 2.64-2.58 (m, 6H), 2.28 (br d, J = 6.4 Hz, |
| | | | | 2H), 2.00 (br d, J = 5.6 Hz, 1H), 1.86 (br d, J = 12.0 Hz, 2H), |
| | | | | 1.78-1.67 (m, 1H), 1.47 (s, 3H), 1.36-1.24 (m, 2H) |
| I-207 | CN | W | 756.5 | 11.96 (s, 1H), 11.06 (s, 1H), 8.51 (d, 1H, J = 52 Hz), 8.20- |
| | | | | 8.10 (m, 2H), 7.80 (s, 2H), 7.51 (d, 1H, J = 5.2 Hz), 7.15 (d, |
| | | | | 1H, J = 2.4 Hz), 7.05 (s, 1H), 6.92 (s, 1H), 6.83 (d, 1H, J = |
| | | | | 1.6 Hz), 6.64 (dd, 1H, J = 1.6, 8.8 Hz), 5.30 (s, 1H), 3.60 (d, |
| | | | | 2H, J = 11.2 Hz), 3.43 (m, 7H), 3.09 (s, 3H), 2.88 (t, 3H, J = |
| | | | | 6.8 Hz), 2.70-2.80 (m, 8H), 2.38 (s, 6H), 2.29 (d, 2H, J = 6.8 |
| | | | | Hz), 2.2-2.0 (m, 1H), 1.85 (d, 2H, J = 11.2 Hz), 1.80-1.60 (m, |
| | | | | 1H), 1.30-1.20 (m, 2H) |
| I-210^{b} | AD | CO | 710.3 | 12.18-11.80 (m, 1H), 10.54 (s, 1H), 9.50-9.14 (m, 2H), |
| | | | | 8.61-8.41 (m, 2H), 8.21-8.04 (m, 3H), 7.77-7.71 (m, 1H), |
| | | | | 7.65-7.49 (m, 2H), 7.31-7.08 (m, 5H), 4.15-3.96 (m, 2H), |
| | | | | 3.94-3.87 (m, 1H), 3.70 (td, J = 6.0, 12.0 Hz, 3H), 3.51- |
| | | | | 3.42 (m, 4H), 3.28 (br s, 6H), 3.00-2.87 (m, 5H), 2.80-2.73 |
| | | | | (m, 1H), 2.17-2.06 (m, 1H), 2.00 (br d, J = 12.0 Hz, 2H), |
| | | | | 1.75-1.61 (m, 2H) |
| I-211^{b} | V | CO | 736.3 | 11.36-11.10 (m, 1H), 10.54 (s, 1H), 9.41 (s, 1H), 9.36-9.23 |
| | | | | (m, 1H), 8.57-8.47 (m, 2H), 8.20-8.07 (m, 3H), 7.78-7.70 |
| | | | | (m, 1H), 7.66-7.50 (m, 2H), 7.30-7.27 (m, 1H), 7.32-7.12 |
| | | | | (m, 4H), 4.09-3.96 (m, 2H), 3.88 (br s, 1H), 3.75-3.66 (m, |
| | | | | 3H), 3.52-3.39 (m, 3H), 3.28-3.21 (m, 7H), 3.01-2.87 (m, |
| | | | | 3H), 2.79-2.73 (m, 1H), 2.16-2.09 (m, 1H), 2.03-1.97 (m, |
| | | | | 2H), 1.74-1.62 (m, 2H), 1.31-1.25 (m, 2H), 1.07-1.00 (m, |
| | | | | 2H) |
| I-212 | CH | CJ | 736.5 | 11.13 (br s, 1H), 10.53 (s, 1H), 9.43 (s, 1H), 8.53 (s, 1H), |
| | | | | 8.46 (d, J = 5.2 Hz, 1H), 8.06 (s, 1H), 8.03 (d, J = 8.8 Hz, |
| | | | | 2H), 7.76-7.69 (m, 1H), 7.62 (d, J = 8.4 Hz, 1H), 7.47 (dd, J = |
| | | | | 1.2, 5.2 Hz, 1H), 7.29 (d, J = 7.6 Hz, 1H), 7.11 (d, J = 2.0 |
| | | | | Hz, 2H), 7.04 (br d, J = 8.8 Hz, 2H), 3.95-3.88 (m, 1H), 3.88- |
| | | | | 3.82 (m, 2H), 3.70 (td, J = 6.4, 12.1 Hz, 1H), 3.29 (s, 1H), |
| | | | | 3.27 (d, J = 2.0 Hz, 2H), 3.21-3.09 (m, 4H), 2.96 (ddd, J = |
| | | | | 6.4, 9.9, 16.4 Hz, 1H), 2.82-2.77 (m, 2H), 2.76-2.69 (m, |
| | | | | 4H), 2.34-2.31 (m, 2H), 1.87 (br d, J = 12.8 Hz, 2H), 1.83- |
| | | | | 1.76 (m, 1H), 1.31-1.23 (m, 4H), 1.03-0.98 (m, 2H) |
| I-213 | CP | w | 769.5 | 11.66 (s, 1H), 11.06 (s, 1H), 8.96 (d, J = 2.4 Hz, 1H), 8.52 (d, |
| | | | | J = 5.2 Hz, 1H), 8.31 (dd, J = 92, 2.0 Hz, 1H), 8.21 (s, 1H), |
| | | | | 7.58 (d, J = 5.2 Hz, 1H), 7.11-7.23 (m, 2H), 6.95 (t, J = 8.8 |
| | | | | Hz, 2H), 6.83 (d, J = 1.6 Hz, 1H), 6.65 (dd, J = 8.8, 1.6 Hz, |
| | | | | 1H), 5.29 (dd, J = 12.8, 5.2 Hz, 1H), 3.62 (br s, 6H), 3.47 (br |
| | | | | d, J = 2.0 Hz, 2H), 3.32-3.33 (m, 3H), 3.31 (s, 3H), 2.84- |
| | | | | 2.96 (m, 1H), 2.59-2.72 (m, 5H), 2.53 (br d, J = 2.0 Hz, |
| | | | | 3H), 2.46-2.48 (m, 2H), 2.25 (br d, J = 7.2 Hz, 2H), 1.95- |
| | | | | 2.11 (m, 5H), 1.85 (br d, J = 12.4 Hz, 2H), 1.70 (br dd, J = |
| | | | | 6.4, 3.2 Hz, 1H), 1.29 (br d, J = 10.0 Hz, 2H) |
| I-215^{b} | J | CU | 745.4 | 11.09 (s, 1H), 11.02-10.88 (m, 1H), 9.34-9.18 (m, 1H), |
| | | | | 8.26 (d, J = 8.8 Hz, 2H), 8.17 (d, J = 8.0 Hz, 1H), 8.11 (d, J = |
| | | | | 3.6 Hz, 1H), 8.09 (s, 1H), 7.22-7.17 (m, 2H), 7.08-7.04 (m, |
| | | | | 1H), 7.00 (d, J = 2.4 Hz, 1H), 6.74 (dd, J = 2.4, 8.8 Hz, 1H), |
| | | | | 5.35 (dd, J = 5.2, 12.8 Hz, 1H), 4.52-4.42 (m, 2H), 4.10- |
| | | | | 4.03 (m, 2H), 3.74-3.69 (m, 3H), 3.64-3.60 (m, 4H), 3.34 |
| | | | | (s, 4H), 2.97-2.84 (m, 2H), 2.72 (d, J = 4.8 Hz, 1H), 2.64 (d, |
| | | | | J = 5.2 Hz, 1H), 2.54 (s, 2H), 2.52 (s, 2H), 2.04-1.96 (m, |
| | | | | 1H), 1.23 (s, 1H), 1.20 (d, J = 6.8 Hz, 3H) |
| I-218 | AT | W | 772.4 | 11.19 (s, 1H), 11.07 (s, 1H), 8.54 (d, J = 5.2 Hz, 1H), 7.90 (s, |
| | | | | 1H), 7.82 (t, J = 9.2 Hz, 1H), 7.54 (d, J = 5.2 Hz, 1H), 7.14 |
| | | | | (s, 1H), 6.99 (d, J = 2.0 Hz, 1H), 6.93 (d, J = 8.8 Hz, 1H), |
| | | | | 6.91-6.88 (m, 1H), 6.83 (d, J = 1.2 Hz, 2H), 6.66 (dd, J = |
| | | | | 8.4, 1.2 Hz, 1H), 5.29 (dd, J = 12.8, 5.2 Hz, 1H), 3.59 (d, J = |
| | | | | 11.6 Hz, 2H), 3.31-3.27 (m, 8H), 2.94-2.82 (m, 1H), 2.70- |
| | | | | 2.54 (m, 9H), 2.25 (d, J = 6.8 Hz, 2H), 2.03-1.94 (m, 1H), |
| | | | | 1.83 (d, J = 11.6 Hz, 2H), 1.73-1.65 (m, 1H), 1.32-1.27 (m, |
| | | | | 5H), 0.99 (t, J = 6.0 Hz, 2H) |
| I-221 | AQ | DA | 769.4 | 11.08 (s, 1H), 9.49 (d, J = 8.0 Hz, 1H), 8.38-8.31 (m, 3H), |
| | | | | 8.28 (d, J = 8.8 Hz, 2H), 8.19 (d, J = 3.2 Hz, 1H), 7.25 (d, J = |
| | | | | 9.2 Hz, 2H), 7.10 (s, 1H), 7.04 (d, J = 8.0 Hz, 1H), 6.92 (d, J = |
| | | | | 8.4 Hz, 1H), 5.35 (dd, J = 5.2, 12.8 Hz, 1H), 3.67-3.59 (m, |
| | | | | 2H), 3.54 (d, J = 11.2 Hz, 2H), 3.48 (s, 2H), 3.42 (d, J = 16.8 |
| | | | | Hz, 2H), 3.37 (s, 1H), 3.35 (s, 3H), 3.31 (d, J = 5.2 Hz, 2H), |
| | | | | 3.21-3.13 (m, 2H), 2.94-2.86 (m, 1H), 2.84-2.79 (m, 1H), |
| | | | | 2.69-2.66 (m, 1H), 2.65 (s, 1H), 2.59 (s, 1H), 2.57 (s, 1H), |
| | | | | 2.54 (s, 1H), 2.04-1.94 (m, 4H), 1.23 (s, 1H), 1.20 (d, J = 6.8 |
| | | | | Hz, 3H) |
| I-222^{b} | AQ | CZ | 769.2 | 11.09 (s, 1H), 10.95-10.81 (m, 1H), 9.55-9.40 (m, 1H), |
| | | | | 8.33 (s, 2H), 8.28 (d, J = 8.8 Hz, 2H), 8.19 (d, J = 2.8 Hz, |
| | | | | 1H), 7.25 (d, J = 8.8 Hz, 2H), 7.14 (s, 1H), 7.10-7.03 (m, |
| | | | | 1H), 6.96 (d, J = 8.0 Hz, 1H), 5.36 (dd, J = 5.6, 12.8 Hz, 1H), |
| | | | | 4.14-4.06 (m, 2H), 3.64 (d, J = 7.6 Hz, 2H), 3.56 (d, J = |
| | | | | 11.6 Hz, 2H), 3.48 (s, 2H), 3.46-3.42 (m, 2H), 3.39 (s, 1H), |
| | | | | 3.37 (s, 3H), 3.26-3.15 (m, 2H), 2.96-2.86 (m, 2H), 2.71 |
| | | | | (dd, J = 4.4, 12.8 Hz, 1H), 2.67-2.59 (m, 1H), 2.54 (s, 1H), |
| | | | | 2.38 (t, J = 7.6 Hz, 4H), 2.04-1.98 (m, 1H), 1.23 (s, 1H), |
| | | | | 1.20 (d, J = 6.8 Hz, 3H) |
| I-226 | BS | W | 746.7 | 11.99 (br s, 1H), 11.07 (s, 1H), 8.53 (d, J = 5.2 Hz, 1H), 8.16 |
| | | | | (s, 1H), 7.90 (s, 1H), 7.83 (t, J = 9.2 Hz, 1H), 7.51 (d, J = 5.2 |
| | | | | Hz, 1H), 7.07 (br s, 1H), 6.98 (d, J = 2.0 Hz, 1H), 6.93 (d, J = |
| | | | | 8.8 Hz, 1H), 6.91-6.86 (m, 1H), 6.83 (d, J = 1.2 Hz, 1H), |
| | | | | 6.64 (dd, J = 1.2, 8.4 Hz, 1H), 5.29 (br dd, J = 5.2, 12.8 Hz, |
| | | | | 1H), 3.59 (br d, J = 11.6 Hz, 3H), 3.44-3.38 (m, 4H), 3.28 |
| | | | | (br s, 5H), 2.94-2.82 (m, 3H), 2.72-2.57 (m, 5H), 2.54 (br |
| | | | | s, 1H), 2.24 (br d, J = 6.8 Hz, 2H), 2.03-1.94 (m, 1H), 1.83 |
| | | | | (br d, J = 12.8 Hz, 2H), 1.73-1.65 (m, 1H), 1.28 (q, J = 11.2 |
| | | | | Hz, 2H) |
| I-230 | DC | W | 856.6 | 11.06 (s, 1H), 9.20 (d, J = 9.2 Hz, 1H), 8.22 (d, J = 8.8 Hz, |
| | | | | 1H), 8.13 (d, J = 9.2 Hz, 1H), 8.08 (d, J = 3.6 Hz, 1H), 8.02 |
| | | | | (d, J = 8.8 Hz, 1H), 7.44 (s, 1H), 7.26 (s, 1H), 7.16 (m, 1H), |
| | | | | 6.94 (d, J = 8.4 Hz, 1H), 6.83 (s, 1H), 6.65 (d, J = 8.4 Hz, |
| | | | | 1H), 6.59 (s, 1H), 5.29 (m, 1H), 4.77 (m, 1H), 3.61 (m, 3H), |
| | | | | 3.48 (s, 2H), 3.31-3.30 (m, 2H), 3.28 (s, 3H), 2.96-2.84 (m, |
| | | | | 1H), 2.70-2.62 (m, 4H), 2.55 (m, 6H), 2.27 (d, J = 6.8 Hz, |
| | | | | 2H), 2.04-1.96 (m, 1H), 1.85 (d, J = 12.4 Hz, 2H), 1.77- |
| | | | | 1.65 (m, 1H), 1.33 (d, J = 6.0 Hz, 7H), 1.28 (s, 1H), 1.21 (d, J = |
| | | | | 6.8 Hz, 3H) |
| I-231^{c} | DE | W | 840.6 | 9.19 (d, 1H, J = 8.9 Hz), 8.10-8.00 (m, 3H), 7.60 (s, 1H), 7.50 |
| | | | | (s, 1H), 7.00-6.90 (m, 2H), 6.87 (d, 1H, J = 1.8 Hz), 6.79 (dd, |
| | | | | 1H, J = 1.6, 8.6 Hz), 5.28 (dd, 1H, J = 5.6, 12.4 Hz), 3.74 (t, |
| | | | | 1H, J = 6.4 Hz), 3.6-3.6 (m, 4H), 3.40 (s, 3H), 3.36 (s, 4H), |
| | | | | 3.0-2.6 (m, 2H), 2.80-2.70 (m, 8H), 2.40 (d, 2H, J = 6.6 Hz), |
| | | | | 2.20-2.10 (m, 1H), 2.00-1.90 (m, 2H), 1.80-1.70 (m, 1H), |
| | | | | 1.42 (dd, 2H, J = 1.2, 11.2 Hz), 1.40-1.30 (m, 9H |
| I-232 | DF | DI | 798.3 | 11.07 (s, 1H), 9.38 (d, J = 2.0 Hz, 1H), 9.27 (d, J = 9.2 Hz, |
| | | | | 1H), 8.62-8.53 (m, 1H), 8.30 (d, J = 8.8 Hz, 1H), 8.24 (s, |
| | | | | 1H), 8.16 (d, J = 9.2 Hz, 1H), 8.10 (d, J = 4.0 Hz, 1H), 8.03 |
| | | | | (d, J = 8.8 Hz, 1H), 7.47 (d, J = 8.4 Hz, 1H), 7.18 (t, J = 4.4 |
| | | | | Hz, 1H), 6.95 (d, J = 8.8 Hz, 1H), 6.86 (d, J = 1.6 Hz, 1H), |
| | | | | 6.70-6.61 (m, 1H), 5.35-5.25 (m, 1H), 3.64-3.60 (m, 1H), |
| | | | | 3.49 (s, 2H), 3.25 (s, 3H), 3.12 (s, 4H), 2.94-2.89 (m, 1H), |
| | | | | 2.80-2.74 (m, 2H), 2.66 (d, J = 15.2 Hz, 4H), 2.23 (d, J = |
| | | | | 6.4 Hz, 2H), 1.97 (d, J = 10.8 Hz, 5H), 1.70-1.60 (m, 3H), |
| | | | | 1.24 (s, 1H), 1.21 (d, J = 6.8 Hz, 3H), 1.14-1.05 (m, 2H) |
| I-233 | DK | W | 826.6 | 11.06 (s, 1H), 9.20 (d, J = 9.2 Hz, 1H), 8.20 (d, J = 8.4 Hz, |
| | | | | 1H), 8.14-8.10 (m, 1H), 8.07 (br d, J = 4.4 Hz, 1H), 8.02 (d, |
| | | | | J = 9.2 Hz, 1H), 7.65 (s, 1H), 7.55 (s, 1H), 7.18-7.12 (m, |
| | | | | 1H), 6.97-6.90 (m, 2H), 6.83 (d, J = 1.6 Hz, 1H), 6.64 (dd, J = |
| | | | | 1.6, 8.8 Hz, 1H), 5.29 (br dd, J = 5.6, 12.8 Hz, 1H), 3.60 (br |
| | | | | d, J = 10.0 Hz, 2H), 3.48 (br d, J = 2.0 Hz, 1H), 3.35 (br s, |
| | | | | 3H), 2.97-2.81 (m, 2H), 2.74-2.68 (m, 3H), 2.63 (br d, J = |
| | | | | 1.2 Hz, 3H), 2.56 (br d, J = 4.0 Hz, 5H), 2.52 (br s, 5H), 2.27 |
| | | | | (br d, J = 7.2 Hz, 3H), 2.04-1.93 (m, 2H), 1.85 (br d, J = |
| | | | | 12.4 Hz, 2H), 1.75-1.67 (m, 1H), 1.30 (br s, 9H) |
| I-234 | DL | G | 785.3 | 11.06 (s, 1H), 9.19 (d, J = 9.2 Hz, 1H), 8.05-8.16 (m, 2H), |
| | | | | 7.96 (d, J = 8.8 Hz, 1H), 7.83 (dd, J = 11.6, 8.8 Hz, 2H), 7.14 |
| | | | | (br t, J = 4.8 Hz, 1H), 6.91 (d, J = 8.4 Hz, 1H), 6.83 (br d, J = |
| | | | | 8.8 Hz, 1H), 6.33 (d, J = 1.6 Hz, 1H), 6.12 (dd, J = 8.4, 1.6 |
| | | | | Hz, 1H), 5.28 (br dd, J = 12.8, 5.2 Hz, 1H), 3.95 (br t, J = 7.2 |
| | | | | Hz, 2H), 3.59 (br s, 5H), 3.44-3.50 (m, 4H), 3.29 (s, 3H), |
| | | | | 2.83-3.03 (m, 3H), 2.64 (br d, J = 5.2 Hz, 4H), 2.55 (br d, |
| | | | | J = 6.8 Hz, 6H), 1.93-2.04 (m, 1H), 1.20 (d, J = 6.8 Hz, 3H) |
| I-235 | FT | G | 804.3 | 11.06 (s, 1H), 9.20 (d, J = 8.8 Hz, 1H), 8.18-8.05 (m, 2H), |
| | | | | 7.97 (d, J = 9.2 Hz, 1H), 7.92 (d, J = 8.8 Hz, 1H), 7.66 (d, J = |
| | | | | 8.8 Hz, 1H), 7.21-7.07 (m, 3H), 6.91 (d, J = 8.4 Hz, 1H), |
| | | | | 6.33 (d, J = 1.2 Hz, 1H), 6.12 (d, J = 8.4 Hz, 1H), 5.33-5.21 |
| | | | | (m, 1H), 3.95 (t, J = 7.2 Hz, 2H), 3.65-3.58 (m, 1H), 3.51- |
| | | | | 3.43 (m, 4H), 3.31-3.27 (m, 6H), 3.01-2.92 (m, 1H), 2.91- |
| | | | | 2.85 (m, 1H), 2.69 (d, J = 4.2 Hz, 1H), 2.64 (d, J = 7.6 Hz, |
| | | | | 4H), 2.55 (d, J = 5.2 Hz, 4H), 2.02-1.94 (m, 1H), 1.20 (d, J = |
| | | | | 6.8 Hz, 3H) |
| I-236 | DN | G | 784.6 | 11.06 (s, 1H), 9.17 (d, J = 9.2 Hz, 1H), 8.13-8.04 (m, 2H), |
| | | | | 7.95 (d, J = 8.8 Hz, 1H), 7.77 (d, J = 8.8 Hz, 1H), 7.52-7.46 |
| | | | | (m, 1H), 7.14 (t, J = 4.4 Hz, 1H), 6.99-6.87 (m, 3H), 6.33 (d, |
| | | | | J = 1.6 Hz, 1H), 6.21-6.07 (m, 1H), 5.36-5.19 (m, 1H), |
| | | | | 3.95 (t, J = 7.2 Hz, 2H), 3.65-3.58 (m, 1H), 3.51-3.43 (m, |
| | | | | 4H), 3.29 (s, 3H), 3.24 (s, 3H), 3.02-2.92 (m, 1H), 2.91- |
| | | | | 2.84 (m, 1H), 2.70-2.67 (m, 1H), 2.64 (d, J = 7.2 Hz, 4H), |
| | | | | 2.56 (d, J = 4.4 Hz, 4H), 2.46 (s, 3H), 2.03-1.93 (m, 1H), |
| | | | | 1.20 (d, J = 6.8 Hz, 3H) |
| I-237 | DO | G | 788.5 | 11.05 (br t, J = 9.6 Hz, 1H), 9.18 (d, J = 8.8 Hz, 1H), 8.14- |
| | | | | 8.06 (m, 3H), 7.99 (s, 1H), 7.97 (s, 1H), 7.13 (br t, J = 4.8 Hz, |
| | | | | 1H), 7.01-6.95 (m, 1H), 6.93-6.84 (m, 2H), 6.33 (d, J = 2.0 |
| | | | | Hz, 1H), 6.12 (dd, J = 2.0, 8.4 Hz, 1H), 5.28 (br dd, J = 5.2, |
| | | | | 12.8 Hz, 1H), 3.95 (br t, J = 7.2 Hz, 2H), 3.62 (br d, J = 3.2 |
| | | | | Hz, 1H), 3.50-3.44 (m, 4H), 3.29 (s, 3H), 3.00-2.93 (m, |
| | | | | 1H), 2.92-2.84 (m, 1H), 2.70-2.67 (m, 1H), 2.65 (br d, J = |
| | | | | 7.2 Hz, 3H), 2.56 (br s, 3H), 2.55 (br s, 2H), 2.53 (br s, 2H), |
| | | | | 2.53 (br s, 1H), 2.03-1.94 (m, 1H), 1.20 (d, J = 6.4 Hz, 3H) |
| I-238 | BW | DV | 742.5 | 11.92 (br s, 1H), 11.09 (br s, 1H), 8.46 (d, J = 5.6 Hz, 1H), |
| | | | | 8.11-7.98 (m, 3H), 7.44 (br d, J = 5.6 Hz, 1H), 7.10-6.87 |
| | | | | (m, 7H), 5.35 (br dd, J = 4.8, 12.8 Hz, 1H), 3.89-3.75 (m, |
| | | | | 3H), 3.63 (s, 3H), 3.00-2.84 (m, 7H), 2.81-2.69 (m, 4H), |
| | | | | 2.65-2.57 (m, 5H), 2.33 (br s, 2H), 2.03-1.96 (m, 1H), 1.84 |
| | | | | (br d, J = 11.6 Hz, 3H), 1.25 (br d, J = 6.4 Hz, 3H), 1.21 (br s, |
| | | | | 1H) |
| I-239 | DR | W | 828.4 | 11.07 (s, 1H), 9.21 (d, J = 8.8 Hz, 1H), 8.23 (d, J = 9.2 Hz, |
| | | | | 1H), 8.15-8.11 (m, 1H), 8.09 (br d, J = 4.4 Hz, 1H), 8.03 (d, |
| | | | | J = 8.8 Hz, 1H), 7.46 (s, 1H), 7.29 (s, 1H), 7.16 (br t, J = 5.2 |
| | | | | Hz, 1H), 6.93 (d, J = 8.4 Hz, 1H), 6.86-6.79 (m, 1H), 6.66- |
| | | | | 6.58 (m, 2H), 5.29 (dd, J = 5.2, 12.9 Hz, 1H), 3.87 (s, 3H), |
| | | | | 3.67-3.56 (m, 3H), 3.48 (br s, 2H), 3.31 (s, 3H), 3.29 (br s, |
| | | | | 4H), 3.24 (s, 1H), 2.96-2.84 (m, 1H), 2.68-2.62 (m, 3H), |
| | | | | 2.55 (br d, J = 6.4 Hz, 4H), 2.26 (br d, J = 7.2 Hz, 2H), 2.05- |
| | | | | 1.94 (m, 1H), 1.85 (br d, J = 12.0 Hz, 2H), 1.70 (br dd, J = |
| | | | | 2.4 10.0 Hz, 1H), 1.35-1.26 (m, 2H), 1.21 (d, J = 6.8 Hz, |
| | | | | 3H) |
| I-240 | FV | W | 823.3 | 11.07 (s, 1H), 9.26 (d, J = 9.2 Hz, 1H), 8.36 (d, J = 9.2 Hz, |
| | | | | 1H), 8.20-8.14 (m, 2H), 8.12-8.08 (m, 2H), 8.06 (d, J = 9.2 |
| | | | | Hz, 1H), 7.50 (s, 1H), 7.19 (t, J = 5.2 Hz, 1H), 6.94 (d, J = |
| | | | | 8.4 Hz, 1H), 6.84-6.73 (m, 1H), 6.67-6.58 (m, 1H), 5.34- |
| | | | | 5.25 (m, 1H), 3.64-3.54 (m, 3H), 3.49 (s, 2H), 3.38 (d, J = |
| | | | | 5.2 Hz, 4H), 3.31 (s, 3H), 3.24 (s, 1H), 2.95-2.85 (m, 1H), |
| | | | | 2.70-2.65 (m, 3H), 2.63-2.57 (m, 4H), 2.28 (d, J = 6.8 Hz, |
| | | | | 2H), 2.03-1.95 (m, 1H), 1.85 (d, J = 11.6 Hz, 2H), 1.77- |
| | | | | 1.65 (m, 1H), 1.35-1.26 (m, 2H), 1.21 (d, J = 6.8 Hz, 3H) |
| I-241 | V | CC | 699.4 | 11.16-11.08 (m, 2H), 8.47 (d, J = 5.2 Hz, 1H), 8.10-8.02 |
| | | | | (m, 3H), 7.48 (dd, J = 1.5, 5.2 Hz, 1H), 7.12 (d, J = 2.4 Hz, |
| | | | | 2H), 7.04 (d, J = 9.2 Hz, 2H), 6.99-6.95 (m, 2H), 6.94-6.90 |
| | | | | (m, 1H), 5.41-5.33 (m, 1H), 3.61-3.58 (m, 3H), 3.28 (d, J = |
| | | | | 2.4 Hz, 2H), 3.24 (br s, 4H), 3.00-2.94 (m, 2H), 2.91-2.84 |
| | | | | (m, 1H), 2.76-2.70 (m, 1H), 2.66-2.56 (m, 5H), 2.46-2.42 |
| | | | | (m, 2H), 2.02-1.95 (m, 1H), 1.86-1.79 (m, 2H), 1.31-1.27 |
| | | | | (m, 2H), 1.04-0.99 (m, 2H) |
| I-242 | V | DO | 699.4 | 11.12 (s, 1H), 11.09 (s, 1H), 8.47 (d, J = 5.2 Hz, 1H), 8.10- |
| | | | | 8.01 (m, 3H), 7.48 (dd, J = 1.6, 5.2 Hz, 1H), 7.12 (d, J = 2.4 |
| | | | | Hz, 2H), 7.08 (s, 1H), 7.03 (t, J = 9.2 Hz, 3H), 6.90 (d, J = |
| | | | | 8.4 Hz, 1H), 5.35 (dd, J = 5.2, 12.8 Hz, 1H), 3.37-3.29 (m, |
| | | | | 16H), 3.28-3.28 (m, 1H), 3.27-3.26 (m, 3H), 2.96-2.84 |
| | | | | (m, 1H), 2.77-2.58 (m, 6H), 2.57-2.53 (m, 7H), 2.42-2.28 |
| | | | | (m, 4H), 2.11-1.91 (m, 2H), 1.91-1.72 (m, 2H), 1.34-1.22 |
| | | | | (m, 2H), 1.07-0.95 (m, 2H) |
| I-243 | CK | W | 755.5 | 11.14-11.03 (m, 2H), 8.93 (d, J = 2.4 Hz, 1H), 8.49 (d, J = |
| | | | | 5.4 Hz, 1H), 8.28 (dd, J = 2.4, 9.2 Hz, 1H), 8.11 (s, 1H), 7.51 |
| | | | | (dd, J = 1.6, 5.2 Hz, 1H), 7.19-7.11 (m, 2H), 6.94 (dd, J = |
| | | | | 5.2, 8.8 Hz, 2H), 6.84 (d, J = 2.0 Hz, 1H), 6.65 (dd, J = 2.0, |
| | | | | 8.8 Hz, 1H), 5.36-5.20 (m, 1H), 3.60 (br d, J = 6.0 Hz, 6H), |
| | | | | 3.32-3.31 (m, 3H), 3.29-3.27 (m, 2H), 2.96-2.84 (m, 1H), |
| | | | | 2.66-2.56 (m, 4H), 2.53 (d, J = 2.0 Hz, 4H), 2.24 (br d, J = |
| | | | | 7.2 Hz, 2H), 2.00 (br dd, J = 4.8, 10.8 Hz, 1H), 1.84 (br d, J = |
| | | | | 11.6 Hz, 2H), 1.75-1.67 (m, 1H), 1.33-1.24 (m, 4H), 1.04- |
| | | | | 0.99 (m, 2H) |
| I-244 | V | W | 754.3 | 11.13 (br s, 1H), 11.05 (s, 1H), 8.47 (d, J = 5.4 Hz, 1H), 8.10- |
| | | | | 8.01 (m, 3H), 7.50-7.44 (m, 1H), 7.12 (d, J = 2.0 Hz, 2H), |
| | | | | 7.04 (br d, J = 8.8 Hz, 2H), 6.93 (d, J = 8.8 Hz, 1H), 6.83 (d, |
| | | | | J = 1.6 Hz, 1H), 6.64 (dd, J = 1.6, 8.8 Hz, 1H), 5.28 (br dd, J = |
| | | | | 5.2, 13.2 Hz, 1H), 3.60 (br d, J = 11.6 Hz, 2H), 3.27 (br s, |
| | | | | 7H), 2.91-2.83 (m, 1H), 2.73-2.56 (m, 6H), 2.32 (br d, J = |
| | | | | 1.6 Hz, 2H), 2.25 (br d, J = 7.6 Hz, 2H), 2.05-1.92 (m, 2H), |
| | | | | 1.84 (br d, J = 10.8 Hz, 2H), 1.73-1.67 (m, 1H), 1.34-1.21 |
| | | | | (m, 5H), 1.01 (br t, J = 5.2 Hz, 2H) |
| I-246 | AQ | DT | 755.7 | 11.09 (s, 1H), 9.15 (d, J = 9.2 Hz, 1H), 8.19 (d, J = 8.8 Hz, |
| | | | | 2H), 8.13 (d, J = 9.2 Hz, 1H), 8.10-8.02 (m, 2H), 7.96 (d, J = |
| | | | | 8.8 Hz, 1H), 7.10 (d, J = 9.6 Hz, 4H), 7.07-7.03 (m, 1H), |
| | | | | 7.02-6.97 (m, 1H), 5.37 (dd, J = 5.2, 12.8 Hz, 1H), 3.86- |
| | | | | 3.70 (m, 1H), 3.59 (s, 4H), 3.53-3.39 (m, 3H), 3.25-3.05 |
| | | | | (m, 3H), 2.98-2.63 (m, 5H), 2.60 (d, J = 1.6 Hz, 1H), 2.52 |
| | | | | (d, J = 2.0 Hz, 3H), 2.15-1.92 (m, 4H), 1.20 (d, J = 6.8 Hz, |
| | | | | 3H) |
| I-248 | FF | DV | 740.3 | 11.93 (br s, 1H), 11.08 (br s, 1H), 8.47 (d, J = 5.2 Hz, 1H), |
| | | | | 8.10-8.00 (m, 3H), 7.44 (dd, J = 1.2, 5.2 Hz, 1H), 7.10-6.95 |
| | | | | (m, 7H), 5.37 (br dd, J = 5.2, 12.6 Hz, 1H), 3.93-3.71 (m, |
| | | | | 3H), 3.59 (s, 3H), 3.25-3.19 (m, 1H), 3.02-2.84 (m, 4H), |
| | | | | 2.83-2.70 (m, 3H), 2.69-2.57 (m, 3H), 2.22 (br d, J = 6.8 |
| | | | | Hz, 2H), 2.11-1.96 (m, 3H), 1.87-1.73 (m, 7H), 1.26-1.24 |
| | | | | (m, 3H), 1.23-1.18 (m, 1H). |
| I-249 | DX | DO | 758.3 | 11.10 (s, 1H), 9.21-9.17 (m, 1H), 8.36 (s, 1H), 8.12 (d, J = |
| | | | | 8.8 Hz, 1H), 8.09 (d, J = 4.4 Hz, 1H), 7.98-7.94 (m, 1H), |
| | | | | 7.83 (d, J = 8.8 Hz, 1H), 7.15 (t, J = 5.2 Hz, 1H), 7.09 (d, J = |
| | | | | 1.2 Hz, 1H), 7.02 (d, J = 8.0 Hz, 1H), 6.91 (dd, J = 1.2, 8.0 |
| | | | | Hz, 1H), 6.83 (s, 1H), 5.39-5.32 (m, 1H), 3.60 (s, 4H), 3.50- |
| | | | | 3.45 (m, 2H), 3.34 (s, 3H), 2.96-2.86 (m, 1H), 2.77-2.72 |
| | | | | (m, 1H), 2.67 (dd, J = 6.4, 8.4 Hz, 4H), 2.53 (d, J = 2.0 Hz, |
| | | | | 3H), 2.48 (s, 3H), 2.38-2.34 (m, 2H), 2.05-1.98 (m, 1H), |
| | | | | 1.86-1.78 (m, 2H), 1.20 (d, J = 6.8 Hz, 3H) |
| I-250 | DX | G | 785.6 | 11.10-11.00 (m, 1H), 9.26-9.15 (m, 1H), 8.37 (s, 1H), 8.26 |
| | | | | (s, 1H), 8.12 (d, J = 8.8 Hz, 1H), 8.07 (d, J = 4.0 Hz, 1H), |
| | | | | 7.96 (d, J = 8.8 Hz, 1H), 7.83 (d, J = 8.8 Hz, 1H), 7.18-7.09 |
| | | | | (m, 1H), 6.91 (d, J = 8.4 Hz, 1H), 6.84 (s, 1H), 6.33 (d, J = |
| | | | | 1.6 Hz, 1H), 6.12 (dd, J = 1.6, 8.8 Hz, 1H), 5.27 (dd, J = 5.6, |
| | | | | 12.8 Hz, 1H), 3.96 (t, J = 7.2 Hz, 2H), 3.60 (s, 8H), 3.49- |
| | | | | 3.44 (m, 6H), 3.29 (s, 3H), 3.03-2.93 (m, 2H), 2.91-2.85 |
| | | | | (m, 1H), 2.64 (d, J = 6.8 Hz, 3H), 2.61-2.57 (m, 1H), 2.02- |
| | | | | 1.95 (m, 1H), 1.20 (d, J = 6.8 Hz, 3H) |
| I-252 | DP | DO | 761.2 | 11.10 (s, 1H), 9.17 (d, J = 9.2 Hz, 1H), 8.13-8.06 (m, 3H), |
| | | | | 7.98 (d, J = 8.8 Hz, 2H), 7.13 (br t, J = 5.2 Hz, 1H), 7.08 (s, |
| | | | | 1H), 7.02 (d, J = 8.0 Hz, 1H), 6.97 (dd, J = 2.0, 8.8 Hz, 1H), |
| | | | | 6.93-6.85 (m, 2H), 5.35 (dd, J = 5.2, 12.8 Hz, 1H), 3.63- |
| | | | | 3.59 (m, 1H), 3.47 (br s, 2H), 3.34 (br s, 3H), 3.32-3.31 (m, |
| | | | | 4H), 2.94-2.85 (m, 1H), 2.78-2.70 (m, 1H), 2.70-2.64 (m, |
| | | | | 3H), 2.60 (br s, 2H), 2.53 (br s, 2H), 2.38 (br t, J = 7.2 Hz, |
| | | | | 2H), 2.05-1.97 (m, 1H), 1.86-1.78 (m, 2H), 1.20 (d, J = 6.8 |
| | | | | Hz, 3H) |
| I-253 | DF | DZ | 812.3 | 11.07 (br s, 1H), 9.16 (d, J = 9.2 Hz, 1H), 8.35 (br s, 1H), |
| | | | | 8.25 (s, 1H), 8.20-8.12 (m, 4H), 7.98 (d, J = 92 Hz, 1H), |
| | | | | 7.09 (br d, J = 8.8 Hz, 2H), 6.95 (d, J = 8.8 Hz, 1H), 6.86 (d, |
| | | | | J = 2.0 Hz, 1H), 6.67-6.62 (m, 1H), 5.30 (br dd, J = 5.6, |
| | | | | 12.8 Hz, 1H), 3.94-3.87 (m, 3H), 3.54 (br s, 1H), 3.32 (s, |
| | | | | 3H), 3.22-3.17 (m, 2H), 3.11 (br s, 2H), 2.91 (s, 3H), 2.82 |
| | | | | (br t, J = 12.0 Hz, 3H), 2.73 (br d, J = 2.8 Hz, 1H), 2.70 (br d, |
| | | | | J = 4.4 Hz, 1H), 2.69-2.62 (m, 4H), 2.25 (br d, J = 6.8 Hz, |
| | | | | 2H), 2.00 (br dd, J = 4.8, 10.8 Hz, 1H), 1.85 (br d, J = 12.0 |
| | | | | Hz, 4H), 1.32-1.24 (m, 5H) |
| I-254 | J | G | 771.3 | 11.06 (s, 1H), 9.17 (d, J = 9.2 Hz, 1H), 9.07 (d, J = 2.4 Hz, |
| | | | | 1H), 8.46 (dd, J = 92, 2.4 Hz, 1H), 8.23 (br s, 1H), 8.18 (d, J = |
| | | | | 9.2 Hz, 1H), 8.06-8.12 (m, 2H), 7.96 (d, J = 9.2 Hz, 1H), |
| | | | | 7.13 (br t, J = 4.8 Hz, 1H), 7.01 (d, J = 9.2 Hz, 1H), 6.91 (d, |
| | | | | J = 8.4 Hz, 1H), 6.33 (d, J = 2.0 Hz, 1H), 6.12 (dd, J = 8.4, |
| | | | | 2.0 Hz, 1H), 5.28 (dd, J = 12.8, 5.4 Hz, 1H), 3.95 (br t, J = |
| | | | | 7.2 Hz, 2H), 3.64 (br s, 6H), 3.47 (br d, J = 5.6 Hz, 4H), 3.29 |
| | | | | (s, 3H), 2.97 (td, J = 13.2, 6.4 Hz, 2H), 2.88 (br dd, J = 16.8, |
| | | | | 4.4 Hz, 1H), 2.68-2.70 (m, 1H), 2.64 (br d, J = 7.6 Hz, 3H), |
| | | | | 2.59 (br s, 1H), 1.95-2.01 (m, 1H), 1.20 (d, J = 6.8 Hz, 3H) |
| I-255^{b} | J | ED | 829.5 | 11.08 (s, 1H), 9.17 (d, J = 8.8 Hz, 1H), 9.07 (d, J = 2.4 Hz, |
| | | | | 1H), 8.51-8.44 (m, 1H), 8.32 (s, 2H), 8.18 (d, J = 9.2 Hz, |
| | | | | 1H), 8.12-8.06 (m, 2H), 7.97 (d, J = 8.8 Hz, 1H), 7.14 (t, J = |
| | | | | 4.8 Hz, 1H), 7.01 (d, J = 9.2 Hz, 1H), 6.87 (d, J = 8.8 Hz, |
| | | | | 1H), 6.68 (d, J = 8.8 Hz, 1H), 5.36-5.27 (m, 1H), 3.79 (s, |
| | | | | 3H), 3.66-3.63 (m, 4H), 3.62 (s, 3H), 3.33 (d, J = 11.6 Hz, |
| | | | | 6H), 2.93-2.84 (m, 3H), 2.64-2.59 (m, 1H), 2.55-2.53 (m, |
| | | | | 4H), 2.26 (d, J = 7.2 Hz, 2H), 2.01-1.95 (m, 1H), 1.77 (d, J = |
| | | | | 11.2 Hz, 2H), 1.70-1.61 (m, 1H), 1.36-1.26 (m, 2H), 1.20 |
| | | | | (d, J = 6.8 Hz, 3H) |
| I-256 | EG | EE | 829.8 | 11.08 (s, 1H), 9.16 (d, J = 9.2 Hz, 1H), 9.05 (d, J = 2.4 Hz, |
| | | | | 1H), 8.50-8.39 (m, 1H), 8.15 (d, J = 4.4 Hz, 1H), 8.11-8.05 |
| | | | | (m, 2H), 7.96 (d, J = 9.2 Hz, 1H), 7.15-7.10 (m, 1H), 7.00 |
| | | | | (d, J = 9.2 Hz, 1H), 6.89 (d, J = 8.8 Hz, 1H), 6.70 (d, J = 8.8 |
| | | | | Hz, 1H), 5.37-5.25 (m, 1H), 4.46 (d, J = 12.4 Hz, 2H), 3.80 |
| | | | | (s, 3H), 3.62 (s, 3H), 3.60-3.53 (m, 1H), 3.53-3.46 (m, 4H), |
| | | | | 2.96-2.90 (m, 2H), 2.84-2.76 (m, 4H), 2.67-2.58 (m, 4H), |
| | | | | 2.27-2.16 (m, 4H), 2.02-1.95 (m, 1H), 1.85 (d, J = 11.2 Hz, |
| | | | | 2H), 1.20 (d, J = 6.8 Hz, 3H), 1.18-1.09 (m, 2H) |
| I-260 | DN | DO | 757.6 | 11.09 (s, 1H), 9.17 (d, J = 8.8 Hz, 1H), 8.16-8.02 (m, 2H), |
| | | | | 7.94 (d, J = 8.8 Hz, 1H), 7.76 (d, J = 8.8 Hz, 1H), 7.52-7.44 |
| | | | | (m, 1H), 7.12 (d, J = 4.4 Hz, 1H), 7.08 (s, 1H), 7.02 (d, J = |
| | | | | 7.6 Hz, 1H), 6.95-6.88 (m, 3H), 5.39-5.29 (m, 1H), 3.65- |
| | | | | 3.56 (m, 1H), 3.47 (s, 2H), 3.31-3.29 (m, 3H), 3.26 (d, J = |
| | | | | 2.4 Hz, 4H), 2.94-2.85 (m, 1H), 2.71 (d, J = 1.2 Hz, 1H), |
| | | | | 2.66 (d, J = 8.0 Hz, 4H), 2.61-2.57 (m, 4H), 2.45 (s, 3H), |
| | | | | 2.41 (s, 1H), 2.04-1.96 (m, 1H), 1.82 (d, J = 6.0 Hz, 2H), |
| | | | | 1.19 (d, J = 6.8 Hz, 3H) |
| I-262 | AQ | AP | 756.5 | 10.84-11.32(m, 1H), 9.09-9.24 (m, 1H), 8.20 (br d, J = 8.8 |
| | | | | Hz, 2H), 8.14 (br d, J = 9.2 Hz, 1H), 8.03-8.11 (m, 2H), |
| | | | | 7.97 (d, J = 9.2 Hz, 1H), 7.11 (br d, J = 8.8 Hz, 3H), 6.94- |
| | | | | 7.02 (m, 1H), 6.76 (br d, J = 8.0 Hz, 1H), 6.71 (d, J = 8.0 Hz, |
| | | | | 1H), 5.26-5.39 (m, 1H), 3.90-4.03 (m, 2H), 3.72 (br t, J = |
| | | | | 5.6 Hz, 2H), 3.58-3.63 (m, 4H), 3.48 (br s, 3H), 2.83-2.93 |
| | | | | (m, 1H), 2.63-2.75 (m, 3H), 2.59 (br s, 2H), 2.53-2.56 (m, |
| | | | | 6H), 1.95-2.05 (m, 1H), 1.18-1.26 (m, 3H) |
| I-264 | DF | BQ | 722.5 | 11.06 (s, 1H), 8.86 (d, J = 9.6 Hz, 1H), 7.97 (d, J = 4.4 Hz, |
| | | | | 1H), 7.86 (d, J = 8.8 Hz, 1H), 7.59 (d, J = 8.8 Hz, 1H), 7.25 |
| | | | | (d, J = 9.6 Hz, 1H), 6.98-6.88 (m, 3H), 6.85 (d, J = 2.0 Hz, |
| | | | | 1H), 6.64 (dd, J = 2.0, 8.6 Hz, 1H), 5.30 (dd, J = 5.2, 12.8 Hz, |
| | | | | 1H), 4.54 (d, J = 13.2 Hz, 3H), 3.58 (dt, J = 4.4, 6.8 Hz, 1H), |
| | | | | 3.43 (s, 3H), 3.31 (s, 3H), 3.11 (s, 5H), 2.98-2.88 (m, 4H), |
| | | | | 2.74-2.69 (m, 1H), 2.67 (dd, J = 2.0, 4.0 Hz, 1H), 2.24 (d, J = |
| | | | | 6.4 Hz, 2H), 2.02-1.97 (m, 1H), 1.86 (d, J = 10.4 Hz, 3H), |
| | | | | 1.18 (d, J = 6.4 Hz, 3H) |
| I-265 | AL | W | 722.3 | 11.07 (s, 1H), 8.89 (d, J = 9.6 Hz, 1H), 7.98 (d, J = 4.4 Hz, |
| | | | | 1H), 7.88 (d, J = 8.8 Hz, 1H), 7.60 (d, J = 8.0 Hz, 1H), 7.26 |
| | | | | (d, J = 9.2 Hz, 1H), 6.94 (d, J = 8.0 Hz, 2H), 6.65 (dd, J = |
| | | | | 2.0, 8.8 Hz, 1H), 5.29 (dd, J = 5.2, 12.8 Hz, 1H), 3.70 (s, 4H), |
| | | | | 3.61 (d, J = 11.6 Hz, 4H), 3.43 (s, 3H), 3.24 (s, 1H), 2.96- |
| | | | | 2.84 (m, 2H), 2.66 (d, J = 2.4 Hz, 2H), 2.63 (s, 2H), 2.26 (d, J = |
| | | | | 6.8 Hz, 2H), 2.02-1.97 (m, 1H), 1.85 (d, J = 12.4 Hz, 2H), |
| | | | | 1.74-1.68 (m, 1H), 1.36-1.23 (m, 4H), 1.18 (d, J = 6.8 Hz, |
| | | | | 3H) |
| I-266 | Y | DO | 757.3 | 11.10 (br s, 1H), 9.17 (d, J = 9.0 Hz, 1H), 8.25 (s, 1H), 8.18 |
| | | | | (dd, J = 2.8, 9.0 Hz, 3H), 8.14 (d, J = 8.9 Hz, 1H), 7.99 (d, J = |
| | | | | 9.0 Hz, 1H), 7.09 (d, J = 9.0 Hz, 2H), 7.00-6.96 (m, 2H), |
| | | | | 6.94-6.91 (m, 1H), 5.37 (dd, J = 5.5, 12.6 Hz, 1H), 3.92 (br |
| | | | | t, J = 7.2 Hz, 1H), 3.60 (s, 3H), 3.52 (br d, J = 14.5 Hz, 1H), |
| | | | | 3.31 (br s, 2H), 3.28 (br s, 3H), 3.19 (br dd, J = 8.4, 14.6 Hz, |
| | | | | 1H), 2.97 (br t, J = 7.6 Hz, 2H), 2.91 (s, 3H), 2.76-2.70 (m, |
| | | | | 1H), 2.70-2.66 (m, 1H), 2.63 (br d, J = 4.5 Hz, 1H), 2.60 (br |
| | | | | d, J = 1.6 Hz, 1H), 2.55 (br s, 2H), 2.44 (br t, J = 6.8 Hz, 2H), |
| | | | | 2.01-1.96 (m, 1H), 1.86-1.79 (m, 2H), 1.28 (d, J = 6.5 Hz, |
| | | | | 3H) |
| I-268 | AN | W | 813.4 | 11.07 (s, 1H), 9.18 (d, J = 9.2 Hz, 1H), 9.05 (d, J = 2.4 Hz, |
| | | | | 1H), 8.45 (dd, J = 9.2, 2.4 Hz, 1H), 8.26 (s, 1H), 8.21 (d, J = |
| | | | | 92 Hz, 1H), 8.17 (d, J = 3.2 Hz, 1H), 7.99 (d, J = 8.8 Hz, |
| | | | | 1H), 7.00 (d, J = 92 Hz, 1H), 6.94 (d, J = 8.8 Hz, 1H), 6.84 |
| | | | | (d, J = 2.0 Hz, 1H), 6.65 (dd, J = 8.8, 2.0 Hz, 1H), 5.29 (dd, J = |
| | | | | 12.8, 5.2 Hz, 1H), 3.92 (br t, J = 7.2 Hz, 1H), 3.61-3.67 |
| | | | | (m, 5H), 3.54 (s, 1H), 3.51 (s, 1H), 3.31 (br s, 3H), 3.12- |
| | | | | 3.26 (m, 4H), 2.91 (s, 4H), 2.61-2.73 (m, 5H), 2.25 (br d, J = |
| | | | | 7.2 Hz, 2H), 1.97-2.02 (m, 1H), 1.85 (br d, J = 11.6 Hz, 2H), |
| | | | | 1.67-1.75 (m, 1H), 1.28 (br d, J = 6.4 Hz, 5H) |
| I-269 | AQ | FK | 770.3 | 11.07 (br s, 1H), 9.15 (d, J = 8.8 Hz, 1H), 8.19 (d, J = 8.8 Hz, |
| | | | | 2H), 8.13 (d, J = 92 Hz, 1H), 8.10-8.02 (m, 2H), 7.96 (d, J = |
| | | | | 9.2 Hz, 1H), 7.09 (br d, J = 9.2 Hz, 3H), 6.99-6.92 (m, |
| | | | | 1H), 6.76-6.71 (m, 1H), 6.67 (d, J = 8.4 Hz, 1H), 5.36-5.28 |
| | | | | (m, 1H), 3.98 (br t, J = 7.2 Hz, 2H), 3.65-3.61 (m, 1H), 3.59 |
| | | | | (s, 3H), 3.54 (br t, J = 6.3 Hz, 2H), 3.47 (br s, 2H), 3.29 (br d, |
| | | | | J = 3.6 Hz, 4H), 2.95-2.84 (m, 2H), 2.73-2.69 (m, 1H), |
| | | | | 2.68-2.64 (m, 3H), 2.59-2.56 (m, 4H), 2.04-1.94 (m, 1H), |
| | | | | 1.20 (d, J = 6.8 Hz, 3H) |
| I-272 | EI | W | 813.3 | 11.06 (s, 1H), 9.21 (br d, J = 8.8 Hz, 1H), 9.01 (s, 1H), 8.40 |
| | | | | (s, 1H), 8.19-8.25 (m, 2H), 8.13 (d, J = 8.8 Hz, 1H), 8.08 (br |
| | | | | d, J = 3.6 Hz, 1H), 8.00 (d, J = 8.8 Hz, 1H), 7.15 (br s, 1H), |
| | | | | 6.94 (d, J = 8.4 Hz, 1H), 6.84 (s, 1H), 6.62-6.68 (m, 1H), |
| | | | | 5.29 (br dd, J = 13.2, 5.2 Hz, 1H), 3.61 (br d, J = 12.0 Hz, |
| | | | | 4H), 3.47 (br d, J = 3.6 Hz, 6H), 2.85-2.94 (m, 2H), 2.64 (br |
| | | | | d, J = 9.6 Hz, 6H), 2.39 (s, 4H), 2.28 (br d, J = 7.2 Hz, 2H), |
| | | | | 1.97-2.02 (m, 1H), 1.85 (br d, J = 12.4 Hz, 3H), 1.67-1.75 |
| | | | | (m, 1H), 1.30 (br d, J = 9.6 Hz, 2H), 1.20 (br d, J = 6.4 Hz, |
| | | | | 3H) |
| I-274 | Y | AF | 812.3 | 11.09 (br s, 1H), 9.17 (d, J = 9.2 Hz, 1H), 8.25 (s, 1H), 8.19 |
| | | | | (dd, J = 3.2, 8.8 Hz, 3H), 8.14 (d, J = 9.2 Hz, 1H), 7.99 (d, J = |
| | | | | 9.2 Hz, 1H), 7.10 (br d, J = 8.8 Hz, 2H), 7.01-6.96 (m, |
| | | | | 1H), 6.89 (br dd, J = 7.6, 14.8 Hz, 2H), 5.36 (br dd, J = 5.6, |
| | | | | 12.4 Hz, 1H), 3.97-3.88 (m, 1H), 3.64 (s, 3H), 3.52 (br d, J = |
| | | | | 14.4 Hz, 1H), 3.30 (br s, 3H), 3.23-3.18 (m, 1H), 3.17- |
| | | | | 3.11 (m, 2H), 2.91 (s, 4H), 2.74 (br s, 1H), 2.71 (br d, J = 2.8 |
| | | | | Hz, 1H), 2.68 (br s, 1H), 2.64 (br d, J = 5.2 Hz, 2H), 2.55 (br |
| | | | | s, 4H), 2.29 (br d, J = 6.4 Hz, 2H), 2.04-1.97 (m, 1H), 1.86 |
| | | | | (br d, J = 10.8 Hz, 2H), 1.77-1.66 (m, 1H), 1.40-1.32 (m, |
| | | | | 2H), 1.28 (br d, J = 6.4 Hz, 3H) |
| I-275 | EK | DO | 744.3 | 11.52-11.42 (m, 1H), 11.09 (s, 1H), 9.38 (d, J = 8.8 Hz, 1H), |
| | | | | 8.51 (d, J = 8.8 Hz, 1H), 8.31 (d, J = 6.0 Hz, 1H), 8.23 (d, J = |
| | | | | 8.8 Hz, 1H), 8.15-8.08 (m, 2H), 8.04 (s, 1H), 7.85 (d, J = 5.6 |
| | | | | Hz, 1H), 7.13 (s, 1H), 7.07 (d, J = 8.0 Hz, 1H), 6.95 (d, J = |
| | | | | 8.0 Hz, 1H), 5.37 (dd, J = 5.2, 12.8 Hz, 1H), 4.68 (d, J = 13.6 |
| | | | | Hz, 2H), 3.75-3.62 (m, 6H), 3.50 (s, 2H), 3.35 (s, 3H), 3.24- |
| | | | | 3.20 (m, 1H), 3.18-3.12 (m, 2H), 2.96-2.87 (m, 1H), 2.75- |
| | | | | 2.71 (m, 2H), 2.63 (d, J = 18.4 Hz, 2H), 2.55 (s, 1H), 2.20- |
| | | | | 2.10 (m, 2H), 2.05-1.97 (m, 1H), 1.21 (d, J = 6.8 Hz, 3H) |
| I-276 | EK | AF | 799.4 | 11.08 (s, 1H), 11.03-10.90 (m, 1H), 9.39 (d, J = 9.2 Hz, 1H), |
| | | | | 8.54 (d, J = 8.8 Hz, 1H), 8.32 (d, J = 6.4 Hz, 1H), 8.24 (d, J = |
| | | | | 8.8 Hz, 1H), 8.15-8.05 (m, 3H), 7.87 (d, J = 6.4 Hz, 1H), |
| | | | | 7.03-6.97 (m, 1H), 6.95-6.88 (m, 2H), 5.37 (dd, J = 5.2, |
| | | | | 12.6 Hz, 1H), 4.69 (d, J = 13.6 Hz, 2H), 3.65 (s, 6H), 3.51 (s, |
| | | | | 3H), 3.27 (dd, J = 2.0, 5.6 Hz, 3H), 3.17 (s, 4H), 2.94-2.86 |
| | | | | (m, 1H), 2.82-2.70 (m, 3H), 2.69-2.59 (m, 2H), 2.07-1.97 |
| | | | | (m, 4H), 1.55-1.43 (m, 2H), 1.22 (d, J = 6.4 Hz, 3H) |
| I-277 | DF | EN | 799.3 | 11.06 (s, 1H), 9.27 (d, J = 8.8 Hz, 1H), 8.32-8.27 (m, 1H), |
| | | | | 8.18-8.14 (m, 2H), 8.10 (br d, J = 4.4 Hz, 1H), 8.06 (d, J = |
| | | | | 8.8 Hz, 1H), 7.64 (s, 1H), 7.45 (d, J = 5.6 Hz, 1H), 7.18 (br t, |
| | | | | J = 5.2 Hz, 1H), 6.94 (d, J = 8.4 Hz, 1H), 6.85 (d, J = 1.6 Hz, |
| | | | | 1H), 6.63 (br d, J = 8.8 Hz, 1H), 5.29 (dd, J = 5.4, 12.8 Hz, |
| | | | | 1H), 4.46 (br d, J = 12.4 Hz, 2H), 3.62 (dt, J = 3.6, 6.8 Hz, |
| | | | | 1H), 3.48 (br s, 2H), 3.31 (s, 3H), 3.11 (br s, 4H), 2.96-2.84 |
| | | | | (m, 3H), 2.74-2.58 (m, 2H), 2.57-2.52 (m, 4H), 2.25 (br d, |
| | | | | J = 6.4 Hz, 2H), 2.03-1.96 (m, 1H), 1.86 (br d, J = 10.8 Hz, |
| | | | | 2H), 1.27-1.10 (m, 5H) |
| I-278 | EK | W | 799.6 | 11.10-11.05 (m, 1H), 9.30-9.25 (m, 1H), 8.34-8.28 (m, |
| | | | | 2H), 8.18 (d, J = 8.8 Hz, 1H), 8.11 (d, J = 4.4 Hz, 1H), 8.06 |
| | | | | (d, J = 9.2 Hz, 1H), 7.66 (s, 1H), 7.52 (d, J = 5.2 Hz, 1H), |
| | | | | 7.19 (t, J = 5.2 Hz, 1H), 6.94 (d, J = 8.8 Hz, 1H), 6.85-6.80 |
| | | | | (m, 1H), 6.65 (dd, J = 1.6, 8.4 Hz, 1H), 5.34-5.23 (m, 1H), |
| | | | | 3.70-3.56 (m, 8H), 3.49 (s, 3H), 3.31 (s, 4H), 3.24 (s, 1H), |
| | | | | 2.68-2.61 (m, 4H), 2.26 (d, J = 6.8 Hz, 2H), 2.05-1.94 (m, |
| | | | | 1H), 1.89-1.82 (m, 2H), 1.75-1.68 (m, 1H), 1.29 (d, J = |
| | | | | 12.4 Hz, 2H), 1.21 (d, J = 6.8 Hz, 3H) |
| I-281 | EO | AF | 799.3 | 1.19 (d, J = 6.4 Hz, 3 H) 1.35 (br d, J = 10.4 Hz, 2 H) 1.69 (br s, |
| | | | | 1 H) 1.85 (br d, J = 11.88 Hz, 2 H) 1.87-1.87 (m, 1 H) 1.95- |
| | | | | 2.05 (m, 1 H) 2.26 (br d, J = 4.4 Hz, 2 H) 2.47 (br s, 2 H) 2.62- |
| | | | | 2.75 (m, 4 H) 2.84-2.94 (m, 1 H) 3.12 (br d, J = 9.2 Hz, 2 H) |
| | | | | 3.46 (br s, 4 H) 3.58-3.61 (m, 1 H) 3.63 (s, 3 H) 3.87 (br s, 4 |
| | | | | H) 5.34 (br dd, J = 12.6, 5.44 Hz, 1 H) 6.83-6.92 (m, 2 H) |
| | | | | 6.93-7.02 (m, 1 H) 7.08-7.16 (m, 1 H) 7.96 (d, J = 8.8 Hz, 1 |
| | | | | H) 8.06 (br d, J = 4.2 Hz, 1 H) 8.10 (d, J = 9.0 Hz, 1 H) 8.13- |
| | | | | 8.19 (m, 2 H) 9.18 (br d, J = 9.0 Hz, 1 H) 9.25 (s, 1 H) 11.08 |
| | | | | (s, 1 H) |
| I-283 | DL | W | 813.4 | 11.07 (s, 1H), 9.19 (d, J = 8.8 Hz, 1H), 8.14-8.05 (m, 2H), |
| | | | | 7.96 (d, J = 9.2 Hz, 1H), 7.82 (dd, J = 8.8, 10.8 Hz, 2H), 7.14 |
| | | | | (br t, J = 4.8 Hz, 1H), 6.94 (d, J = 8.8 Hz, 1H), 6.85-6.78 |
| | | | | (m, 2H), 6.65 (dd, J = 2.0, 8.8 Hz, 1H), 5.33-5.26 (m, 1H), |
| | | | | 3.59 (br s, 8H), 3.48 (br d, J = 4.0 Hz, 4H), 3.32 (s, 3H), 2.92- |
| | | | | 2.84 (m, 1H), 2.68-2.61 (m, 3H), 2.56 (s, 3H), 2.48 (br s, |
| | | | | 2H), 2.24 (br d, J = 6.8 Hz, 2H), 2.04-1.95 (m, 1H), 1.84 (br |
| | | | | d, J = 12.0 Hz, 2H), 1.74-1.65 (m, 1H), 1.35-1.24 (m, 2H), |
| | | | | 1.20 (d, J = 6.4 Hz, 3H) |
| I-284 | FF | EQ | 815.5 | 11.11 (s, 1H), 9.24 (br d, J = 8.8 Hz, 1H), 8.22 (s, 1H), 8.16 |
| | | | | (d, J = 8.8 Hz, 1H), 8.10 (br d, J = 4.0 Hz, 1H), 8.04 (br d, J = |
| | | | | 8.8 Hz, 1H), 8.01 (br d, J = 7.2 Hz, 1H), 7.61 (br d, J = 3.6 |
| | | | | Hz, 1H), 7.28-7.21 (m, 1H), 7.17 (br s, 1H), 7.15-7.10 (m, |
| | | | | 1H), 7.05-7.00 (m, 2H), 6.98 (br s, 1H), 5.38 (br dd, J = 4.8, |
| | | | | 12.0 Hz, 1H), 3.71 (br d, J = 11.2 Hz, 2H), 3.59 (s, 3H), 3.00 |
| | | | | (br d, J = 10.0 Hz, 2H), 2.91-2.84 (m, 1H), 2.76-2.71 (m, |
| | | | | 2H), 2.67 (br d, J = 18.4 Hz, 2H), 2.60 (br s, 2H), 2.55 (s, |
| | | | | 2H), 2.24 (br d, J = 6.4 Hz, 2H), 2.13-2.04 (m, 2H), 2.03- |
| | | | | 1.97 (m, 1H), 1.86 (br d, J = 12.0 Hz, 2H), 1.83-1.76 (m, |
| | | | | 4H), 1.73-1.65 (m, 1H), 1.28 (br d, J = 11.2 Hz, 2H), 1.20 |
| | | | | (br d, J = 6.4 Hz, 3H) |
| I-285 | BW | EQ | 816.5 | 11.09 (br s, 1H), 9.24 (d, J = 8.8 Hz, 1H), 8.16 (d, J = 9.2 Hz, |
| | | | | 1H), 8.09 (br d, J = 4.4 Hz, 1H), 8.04 (d, J = 9.2 Hz, 1H), |
| | | | | 8.01 (dd, J = 2.4, 8.8 Hz, 1H), 7.62 (dd, J = 3.2, 6.4 Hz, 1H), |
| | | | | 7.28-7.20 (m, 1H), 7.17 (br t, J = 5.2 Hz, 1H), 7.12 (td, J = |
| | | | | 3.6, 8.8 Hz, 1H), 7.02-6.96 (m, 1H), 6.96-6.92 (m, 1H), |
| | | | | 6.89 (br d, J = 7.2 Hz, 1H), 5.36 (br dd, J = 5.2, 12.4 Hz, 1H), |
| | | | | 3.71 (br d, J = 11.6 Hz, 2H), 3.64 (s, 3H), 3.49 (br s, 2H), |
| | | | | 3.35 (br s, 1H), 3.06-2.84 (m, 6H), 2.78-2.71 (m, 2H), 2.69 |
| | | | | (br d, J = 9.6 Hz, 1H), 2.66-2.60 (m, 1H), 2.52 (br s, 2H), |
| | | | | 2.50 (br s, 1H), 2.28 (br d, J = 7.2 Hz, 2H), 2.04-1.97 (m, |
| | | | | 1H), 1.87 (br d, J = 12.0 Hz, 2H), 1.78-1.67 (m, 1H), 1.36- |
| | | | | 1.26 (m, 2H), 1.20 (d, J = 6.8 Hz, 3H) |
| I-286 | EU | ER | 831.7 | 11.09 (s, 1H), 9.24 (d, J = 9.2 Hz, 1H), 8.19-8.14 (m, 1H), |
| | | | | 8.09 (d, J = 4.0 Hz, 1H), 8.06-7.98 (m, 2H), 7.60 (dd, J = |
| | | | | 3.2, 6.4 Hz, 1H), 7.31-7.21 (m, 1H), 7.16 (t, J = 5.2 Hz, 1H), |
| | | | | 7.14-7.09 (m, 1H), 6.99-6.92 (m, 2H), 6.89-6.83 (m, 1H), |
| | | | | 5.41-5.32 (m, 1H), 3.67-3.60 (m, 1H), 3.56 (s, 3H), 3.48 (d, |
| | | | | J = 3.2 Hz, 2H), 3.18 (s, 4H), 2.92-2.84 (m, 3H), 2.75-2.58 |
| | | | | (m, 3H), 2.55 (s, 1H), 2.38-2.28 (m, 3H), 2.04-1.96 (m, |
| | | | | 1H), 1.65-1.56 (m, 2H), 1.51-1.43 (m, 2H), 1.42-1.27 (m, |
| | | | | 9H), 1.20 (d, J = 6.8 Hz, 3H) |
| I-287 | J | DO | 744.6 | 11.10 (br s, 1H), 9.17 (br d, J = 9.2 Hz, 1H), 9.06 (d, J = 1.6 |
| | | | | Hz, 1H), 8.49-8.43 (m, 1H), 8.18 (br d, J = 8.8 Hz, 1H), 8.12- |
| | | | | 8.06 (m, 2H), 7.96 (d, J = 8.8 Hz, 1H), 7.16-7.11 (m, 1H), |
| | | | | 7.09 (s, 1H), 7.01 (br t, J = 7.6 Hz, 2H), 6.91 (br d, J = 7.6 |
| | | | | Hz, 1H), 5.35 (br dd, J = 5.2, 12.9 Hz, 1H), 3.72-3.55 (m, |
| | | | | 6H), 3.47 (br s, 2H), 3.32 (br s, 3H), 2.96-2.86 (m, 1H), 2.78- |
| | | | | 2.71 (m, 1H), 2.71-2.63 (m, 4H), 2.62-2.58 (m, 2H), 2.05- |
| | | | | 1.98 (m, 1H), 1.87-1.78 (m, 2H), 1.24 (br s, 1H), 1.20 (br |
| | | | | d, J = 6.8 Hz, 3H) |
| I-288 | J | CC | 744.2 | 11.09 (br s, 1H), 9.17 (d, J = 9.2 Hz, 1H), 9.06 (d, J = 2.4 Hz, |
| | | | | 1H), 8.46 (dd, J = 2.4, 8.9 Hz, 1H), 8.17 (d, J = 9.2 Hz, 1H), |
| | | | | 8.10 (d, J = 8.8 Hz, 1H), 8.06 (br d, J = 4.0 Hz, 1H), 7.96 (d, |
| | | | | J = 9.2 Hz, 1H), 7.12 (br t, J = 5.2 Hz, 1H), 7.02-6.97 (m, |
| | | | | 3H), 6.94-6.91 (m, 1H), 5.37 (dd, J = 5.2, 12.6 Hz, 1H), 3.62 |
| | | | | (br s, 4H), 3.60 (s, 3H), 3.48 (br s, 2H), 3.30 (br s, 1H), 2.98 |
| | | | | (br t, J = 7.2 Hz, 2H), 2.88 (br dd, J = 5.2, 16.8 Hz, 1H), 2.76- |
| | | | | 2.69 (m, 1H), 2.64 (br d, J = 4.4 Hz, 1H), 2.60 (br s, 1H), |
| | | | | 2.53-2.52 (m, 2H), 2.44 (br t, J = 6.8 Hz, 2H), 2.03-1.96 |
| | | | | (m, 1H), 1.86-1.79 (m, 2H), 1.20 (d, J = 6.8 Hz, 3H) |
| I-289 | DP | W | 816.3 | 11.07 (s, 1H), 9.17 (d, J = 9.2 Hz, 1H), 8.13-8.06 (m, 4H), |
| | | | | 7.97 (d, J = 8.8 Hz, 2H), 7.13 (br t, J = 4.8 Hz, 1H), 6.99- |
| | | | | 6.89 (m, 3H), 6.86-6.82 (m, 2H), 6.64 (dd, J = 2.0, 8.8 Hz, |
| | | | | 1H), 3.60 (br d, J = 11.2 Hz, 4H), 3.47 (br s, 3H), 3.33-3.32 |
| | | | | (m, 4H), 2.95-2.84 (m, 1H), 2.76-2.55 (m, 8H), 2.24 (br d, |
| | | | | J = 7.2 Hz, 2H), 2.02-1.96 (m, 1H), 1.83 (br d, J = 11.2 Hz, |
| | | | | 2H), 1.33-1.23 (m, 3H), 1.19 (d, J = 6.8 Hz, 3H) |
| I-290 | EW | AF | 742.6 | 1.25 (d, J = 6.4 Hz, 3 H) 1.29-1.42 (m, 2 H) 1.64-1.77 (m, 1 |
| | | | | H) 1.80-1.91 (m, 2 H) 1.96-2.03 (m, 1 H) 2.25-2.35 (m, 3 |
| | | | | H) 2.54 (s, 2 H) 2.57-2.78 (m, 6 H) 2.79-2.96 (m, 2 H) 3.13 |
| | | | | (br d, J = 9.6 Hz, 2 H) 3.26 (br s, 4 H) 3.63 (s, 3 H) 3.74-3.85 |
| | | | | (m, 1 H) 5.30-5.38 (m, 1 H) 6.88 (br dd, J = 16.4, 7.94 Hz, 2 |
| | | | | H) 6.94-7.07 (m, 4 H) 7.10 (d, J = 2.0 Hz, 1 H) 7.45 (dd, |
| | | | | J = 5.2, 1.2 Hz, 1 H) 8.03-8.15 (m, 3 H) 8.47 (d, J = 5.2 Hz, 1 |
| | | | | H) 11.08 (s, 1H) 11.89-11.95 (m, 1 H) |
| I-292 | DF | EY | 832.5 | 11.06 (s, 1H), 9.19 (d, J = 9.2 Hz, 1H), 8.22 (s, 1H), 8.13 (d, J = |
| | | | | 9.2 Hz, 1H), 8.08 (d, J = 4.0 Hz, 1H), 7.97 (d, J = 9.2 Hz, |
| | | | | 1H), 7.92 (d, J = 8.8 Hz, 1H), 7.66 (d, J = 9.2 Hz, 1H), 7.15 |
| | | | | (t, J = 5.2 Hz, 1H), 7.11-7.06 (m, 2H), 7.01-6.91 (m, 1H), |
| | | | | 6.90-6.79 (m, 1H), 6.71-6.55 (m, 1H), 5.40-5.18 (m, 1H), |
| | | | | 3.88 (d, J = 12.0 Hz, 2H), 3.66-3.58 (m, 2H), 3.48 (d, J = |
| | | | | 5.6 Hz, 3H), 3.12 (d, J = 1.2 Hz, 5H), 2.94-2.89 (m, 1H), |
| | | | | 2.89-2.79 (m, 3H), 2.70 (s, 1H), 2.64 (s, 1H), 2.25 (d, J = 6.8 |
| | | | | Hz, 2H), 2.03-1.97 (m, 1H), 1.88-1.80 (m, 3H), 1.32-1.22 |
| | | | | (m, 3H), 1.20 (d, J = 6.4 Hz, 3H) |
| I-293 | FC | EZ | 825.8 | 9.17 (d, J = 9.2 Hz, 1H), 8.34 (s, 1H), 8.15 (d, J = 9.2 Hz, |
| | | | | 1H), 8.12-8.08 (m, 1H), 8.05-8.00 (m, 1H), 7.79 (s, 1H), |
| | | | | 7.60 (d, J = 7.2 Hz, 1H), 7.39 (t, J = 8.0 Hz, 1H), 7.08 (d, J = |
| | | | | 7.6 Hz, 1H), 7.00-6.92 (m, 2H), 6.87 (d, J = 6.8 Hz, 1H), |
| | | | | 5.27 (dd, J = 4.8, 12.8 Hz, 1H), 3.73 (s, 4H), 3.64-3.60 (m, |
| | | | | 1H), 3.52 (s, 3H), 3.46 (d, J = 4.0 Hz, 1H), 3.20 (s, 4H), 2.98 |
| | | | | (d, J = 6.8 Hz, 2H), 2.89-2.82 (m, 3H), 2.62 (s, 2H), 2.54 (s, |
| | | | | 1H), 2.00 (dd, J = 5.6, 10.4 Hz, 1H), 1.89 (s, 4H), 1.56 (d, J = |
| | | | | 6.4 Hz, 2H), 1.44-1.28 (m, 6H), 1.18 (d, J = 6.4 Hz, 3H) |
| I-294 | FB | EZ | 825.5 | 11.08 (br s, 1H), 9.14 (d, J = 9.2 Hz, 1H), 8.17 (d, J = 8.8 |
| | | | | Hz, 2H), 8.12 (d, J = 9.2 Hz, 1H), 8.02-8.09 (m, 2H), 7.95 |
| | | | | (d, J = 9.2 Hz, 1H), 7.05-7.15 (m, 3H), 6.94-6.99 (m, 2H), |
| | | | | 6.84-6.89 (m, 1H), 5.37 (dd, J = 12.8, 5.2 Hz, 1H), 3.58- |
| | | | | 3.66 (m, 1H), 3.56 (s, 3H), 3.47 (br s, 2H), 3.21-3.28 (m, |
| | | | | 4H), 2.85-2.95 (m, 7H), 2.68-2.77 (m, 1H), 2.59-2.67 (m, |
| | | | | 1H), 2.53 (d, J = 2.0 Hz, 1H), 2.37 (br t, J = 6.4 Hz, 2H), |
| | | | | 1.96-2.04 (m, 1H), 1.73-1.82 (m, 4H), 1.54-1.64 (m, 2H), |
| | | | | 1.27-1.43 (m, 6H), 1.20 (d, J = 6.8 Hz, 3H) |
| I-295 | FI | FE | 731.1 | 12.00 (s, 1H), 11.02-10.89(M, 1H), 8.53 (d, J = 5.2 Hz, 1H), |
| | | | | 7.89-7.82 (m, 2H), 7.52-7.49 (m, 2H), 7.07 (d, J = 9.2 Hz, |
| | | | | 1H), 6.98-6.85 (m, 3H), 5.07-5.03 (m, 1H), 4.36-4.19 (m, |
| | | | | 2H), 3.86 (d, J = 12.4 Hz, 2H), 2.92-2.82 (m, 10H), 2.34- |
| | | | | 2.24 (m, 5H), 2.19(dd, J = 6.0 Hz 1H), 1.84-1.81 (m, 5H), |
| | | | | 1.24-1.15 (m, 3H) |
| I-358 | AL | AY | 748 | 11.07 (s, 1H), 8.92-8.88 (m, 1H), 8.16 (s, 1H), 8.01-7.97 |
| | | | | (m, 1H), 7.89 (d, J = 9.2 Hz, 1H), 7.60 (d, J = 9.2 Hz, 1H), |
| | | | | 7.26 (d, J = 9.6 Hz, 1H), 6.95-6.91 (m, 2H), 6.84 (d, J = 2.0 |
| | | | | Hz, 1H), 6.64 (dd, J = 2.0, 8.8 Hz, 1H), 5.29 (dd, J = 5.2, 12.8 |
| | | | | Hz, 1H), 3.69 (s, 4H), 3.58 (td, J = 2.4, 3.6 Hz, 1H), 3.43 (d, J = |
| | | | | 4.4 Hz, 2H), 3.31 (s, 3H), 3.08 (s, 2H), 3.00 (d, J = 1.6 Hz, |
| | | | | 2H), 2.93-2.85 (m, 1H), 2.78-2.72 (m, 1H), 2.71-2.62 (m, |
| | | | | 2H), 2.41 (s, 4H), 2.05-1.98 (m, 3H), 1.70 (d, J = 5.2 Hz, |
| | | | | 2H), 1.68-1.63 (m, 4H), 1.18 (d, J = 6.8 Hz, 3H) |
| I-404 | M | G | 773.7 | 11.63 (br s, 1H), 11.06 (br s, 1H), 8.70 (d, J = 5.2 Hz, 1H), |
| | | | | 8.18 (t, J = 9.2 Hz, 1H), 7.66 (d, J = 5.2 Hz, 1H), 7.17-7.29 |
| | | | | (m, 2H), 6.75-6.87 (m, 1H), 6.81 (dd, J = 15.2, 2.0 Hz, 1H), |
| | | | | 6.32 (d, J = 2.0 Hz, 1H), 6.12 (dd, J = 8.4, 2.0 Hz, 1H), 5.27 |
| | | | | (dd, J = 12.8, 5.2 Hz, 1H), 3.95 (t, J = 7.2 Hz, 2H), 3.45 (br |
| | | | | s, 2H), 3.32 (br s, 4H), 3.29 (s, 3H), 3.17 (br d, J = 2.0 Hz, |
| | | | | 2H), 2.84-3.00 (m, 2H), 2.64 (br d, J = 7.2 Hz, 3H), 2.54 (br |
| | | | | d, J = 3.6 Hz, 5H), 1.94-2.03 (m, 3H), 1.69-1.88 (m, 6H) |
| I-405 | L | G | 755.3 | 11.63 (br s, 1H), 11.05 (s, 1H), 8.66 (d, J = 5.6 Hz, 1H), 8.44 |
| | | | | (d, J = 8.8 Hz, 2H), 7.61 (d, J = 5.2 Hz, 1H), 7.27 (s, 1H), |
| | | | | 7.22 (br s, 1H), 7.06 (br d, J = 8.8 Hz, 2H), 6.91 (d, J = 8.4 |
| | | | | Hz, 1H), 6.33 (s, 1H), 6.07-6.15 (m, 1H), 5.23-5.31 (m, |
| | | | | 1H), 3.95 (t, J = 7.6 Hz, 2H), 3.46 (br s, 2H), 3.29 (br s, 3H), |
| | | | | 3.18 (br s, 2H), 2.83-3.01 (m, 3H), 2.63-2.69 (m, 6H), 1.96- |
| | | | | 2.10 (m, 4H), 1.78-1.91 (m, 5H), 1.69-1.77 (m, 2H), 1.24 |
| | | | | (br d, J = 1.6 Hz, 1H) |
| I-410 | Q | R | 762.4 | 11.19-10.95 (m, 1H), 8.87 (d, J = 9.6 Hz, 1H), 7.97 (br d, J = |
| | | | | 4.4 Hz, 1H), 7.87 (d, J = 8.8 Hz, 1H), 7.59 (d, J = 8.8 Hz, |
| | | | | 1H), 7.26 (d, J = 9.6 Hz, 1H), 6.95-6.88 (m, 2H), 6.83 (d, J = |
| | | | | 2.0 Hz, 1H), 6.64 (dd, J = 1.6, 8.8 Hz, 1H), 5.29 (dd, J = |
| | | | | 5.2, 12.8 Hz, 1H), 4.42-4.31 (m, 2H), 3.62-3.53 (m, 1H), |
| | | | | 3.43 (br d, J = 4.0 Hz, 2H), 3.30 (s, 3H), 3.14-3.01 (m, 6H), |
| | | | | 2.94-2.84 (m, 1H), 2.69-2.61 (m, 4H), 2.59 (br s, 1H), 2.53 |
| | | | | (br s, 1H), 2.31-2.25 (m, 1H), 2.01-1.90 (m, 3H), 1.69- |
| | | | | 1.57 (m, 6H), 1.48-1.40 (m, 2H), 1.18 (d, J = 6.4 Hz, 3H) |
| I-411 | AI | BB | 762.4 | 11.04 (s, 1H), 8.84 (d, J = 9.6 Hz, 1H), 7.94 (d, J = 4.0 Hz, |
| | | | | 1H), 7.83 (d, J = 8.8 Hz, 1H), 7.55 (d, J = 8.8 Hz, 1H), 7.07 |
| | | | | (d, J = 9.6 Hz, 1H), 6.92-6.88 (m, 2H), 6.79 (d, J = 2.0 Hz, |
| | | | | 1H), 6.56 (d, J = 8.4 Hz, 1H), 5.27 (dd, J = 5.2, 13.2 Hz, 1H), |
| | | | | 3.89-3.83 (m, 2H), 3.79 (t, J = 5.6 Hz, 2H), 3.60-3.54 (m, |
| | | | | 1H), 3.41 (s, 2H), 3.29 (s, 3H), 3.04-3.00 (m, 2H), 2.90 (d, J = |
| | | | | 7.2 Hz, 4H), 2.67 (d, J = 2.0 Hz, 1H), 2.59 (s, 2H), 2.39- |
| | | | | 2.36 (m, 2H), 2.02-1.94 (m, 4H), 1.91 (d, J = 4.0 Hz, 2H), |
| | | | | 1.64 (d, J = 5.2 Hz, 2H), 1.58-1.47 (m, 5H), 1.17 (d, J = 6.8 |
| | | | | Hz, 3H) |
| I-412 | AN | G | 785.1 | 11.05 (s, 1H), 9.18 (d, J = 9.2 Hz, 1H), 9.05 (d, J = 2.4 Hz, |
| | | | | 1H), 8.46 (dd, J = 9.2, 2.4 Hz, 1H), 8.25 (br d, J = 2.4 Hz, |
| | | | | 1H), 8.21 (d, J = 9.2 Hz, 1H), 8.15 (d, J = 9.2 Hz, 1H), 7.99 |
| | | | | (d, J = 8.8 Hz, 1H), 7.01 (d, J = 9.2 Hz, 1H), 6.91 (d, J = 8.4 |
| | | | | Hz, 1H), 6.33 (d, J = 2.0 Hz, 1H), 6.12 (dd, J = 8.4, 2.0 Hz, |
| | | | | 1H), 5.27 (dd, J = 12.8, 5.6 Hz, 1H), 3.88-4.01 (m, 3H), |
| | | | | 3.64 (br s, 4H), 3.53 (br d, J = 14.4 Hz, 1H), 3.47 (br t, J = |
| | | | | 6.4 Hz, 2H), 3.29 (s, 3H), 3.16-3.22 (m, 1H), 2.85-3.01 (m, |
| | | | | 5H), 2.62-2.74 (m, 4H), 2.54-2.61 (m, 2H), 1.94-2.03 (m, |
| | | | | 1H), 1.29 (d, J = 6.8 Hz, 3H) |
| I-413 | Y | X | 743.4 | 11.10 (s, 1H), 9.17 (d, J = 8.8 Hz, 1H), 8.26-8.12 (m, 6H), |
| | | | | 8.01-7.97 (m, 1H), 7.11 (d, J = 8.8 Hz, 2H), 7.01-6.93 (m, |
| | | | | 3H), 5.42-5.34 (m, 1H), 3.96-3.90 (m, 1H), 3.62 (s, 3H), |
| | | | | 3.52 (d, J = 14.4 Hz, 2H), 3.23-3.10 (m, 6H), 2.91 (s, 3H), |
| | | | | 2.70-2.65 (m, 8H), 2.01 (dd, J = 5.2, 10.8 Hz, 1H), 1.28 (d, J = |
| | | | | 6.4 Hz, 3H) |
| I-414 | AN | X | 744.3 | 11.10 (s, 1H), 9.19 (d, J = 8.8 Hz, 1H), 9.07 (s, 1H), 8.51- |
| | | | | 8.45 (m, 1H), 8.27-8.13 (m, 3H), 8.00 (d, J = 9.2 Hz, 1H), |
| | | | | 7.05-6.94 (m, 4H), 5.39 (dd, J = 4.8, 12.4 Hz, 1H), 3.96- |
| | | | | 3.90 (m, 1H), 3.63 (s, 3H), 3.53 (d, J = 14.8 Hz, 2H), 3.33- |
| | | | | 3.31 (m, 8H), 3.23-3.16 (m, 2H), 2.91 (s, 3H), 2.77-2.69 |
| | | | | (m, 2H), 2.68-2.65 (m, 2H), 2.05-1.96 (m, 2H), 1.29 (d, J = |
| | | | | 6.8 Hz, 3H) |
| I-415 | AB | G | 754.4 | 11.46 (br s, 1H), 11.06 (s, 1H), 8.49 (d, J = 5.2 Hz, 1H), 8.15 |
| | | | | (br s, 1H), 8.07 (br d, J = 8.8 Hz, 2H), 7.54 (dd, J = 5.2, 1.0 |
| | | | | Hz, 1H), 7.15 (br s, 1H), 7.10 (d, J = 2.0 Hz, 1H), 7.06 (br d, |
| | | | | J = 8.8 Hz, 2H), 6.91 (d, J = 8.4 Hz, 1H), 6.33 (d, J = 1.6 Hz, |
| | | | | 1H), 6.12 (dd, J = 8.4, 2.0 Hz, 1H), 5.28 (br dd, J = 12.4, 5.2 |
| | | | | Hz, 1H), 3.96 (br t, J = 7.2 Hz, 2H), 3.47 (br t, J = 6.4 Hz, |
| | | | | 2H), 3.29 (s, 4H), 3.18 (br d, J = 2.0 Hz, 2H), 2.82-3.02 (m, |
| | | | | 3H), 2.64-2.71 (m, 3H), 2.59 (br s, 3H), 1.92-2.00 (m, 3H), |
| | | | | 1.77-1.89 (m, 5H), 1.73 (br dd, J = 6.4, 4.4 Hz, 2H), 1.57- |
| | | | | 1.65 (m, 1H), 1.19-1.29 (m, 1H), 0.90 (t, J = 7.2 Hz, 1H) |
| I-417 | CF | G | 716.1 | 8.88 (d, J = 2.4 Hz, 1H), 8.63 (br d, J = 2.4 Hz, 1H), 8.53 (d, |
| | | | | J = 5.2 Hz, 1H), 8.24 (br dd, J = 9.2, 2.4 Hz, 1H), 7.97 (s, |
| | | | | 1H), 7.56 (s, 1H), 7.29 (dd, J = 5.2, 1.2 Hz, 1H), 6.92 (dd, J = |
| | | | | 15.2, 8.8 Hz, 2H), 6.31 (d, J = 2.0 Hz, 1H), 6.11 (dd, J = |
| | | | | 8.4, 2.0 Hz, 1H), 5.27 (dd, J = 12.8, 5.2 Hz, 1H), 3.94 (br t, J = |
| | | | | 7.2 Hz, 2H), 3.84-3.91 (m, 1H), 3.59 (br s, 4H), 3.45 (br t, |
| | | | | J = 6.4 Hz, 2H), 3.28 (s, 3H), 2.92-2.99 (m, 1H), 2.82-2.91 |
| | | | | (m, 1H), 2.58-2.70 (m, 4H), 2.54 (br s, 2H), 2.45 (dt, J = |
| | | | | 2.4, 1.2 Hz, 2H), 2.40 (dd, J = 16.4, 4.8 Hz, 1H), 2.24-2.32 |
| | | | | (m, 1H), 1.94-2.01 (m, 1H), 1.28 (d, J = 6.4 Hz, 3H) |
| I-418 | AB | AP | 740.4 | 11.44 (s, 1H), 11.09 (s, 1H), 8.49 (d, J = 5.2 Hz, 1H), 8.15 (s, |
| | | | | 1H), 8.07 (d, J = 8.8 Hz, 2H), 7.53 (dd, J = 5.6, 1.2 Hz, 1H), |
| | | | | 7.15 (br s, 1H), 7.09 (d, J = 2.4 Hz, 1H), 7.06 (br d, J = 9.2 |
| | | | | Hz, 2H), 6.95-7.01 (m, 1H), 6.76 (d, J = 8.0 Hz, 1H), 6.71 |
| | | | | (d, J = 8.0 Hz, 1H), 5.34 (br dd, J = 12.8, 5.2 Hz, 1H), 3.96 |
| | | | | (br d, J = 3.2 Hz, 2H), 3.69-3.74 (m, 2H), 3.60 (s, 3H), 3.28 |
| | | | | (br d, J = 5.6 Hz, 4H), 3.18 (d, J = 2.4 Hz, 2H), 2.84-2.93 |
| | | | | (m, 1H), 2.57-2.77 (m, 3H), 2.53 (br s, 3H), 1.92-2.01 (m, |
| | | | | 3H), 1.77-1.90 (m, 5H), 1.69-1.77 (m, 2H) |
| I-419 | V | AP | 712.4 | 11.19-11.04 (m, 2H), 8.48 (d, J = 5.2 Hz, 1H), 8.11-8.01 |
| | | | | (m, 3H), 7.48 (dd, J = 1.6, 5.2 Hz, 1H), 7.12 (d, J = 2.4 Hz, |
| | | | | 2H), 7.06 (br d, J = 9.2 Hz, 2H), 7.01-6.95 (m, 1H), 6.76 (d, |
| | | | | J = 7.6 Hz, 1H), 6.71 (d, J = 8.0 Hz, 1H), 5.33 (br dd, J = 5.2, |
| | | | | 12.8 Hz, 1H), 4.00-3.93 (m, 2H), 3.72 (br t, J = 5.6 Hz, 2H), |
| | | | | 3.60 (s, 3H), 3.28 (br d, J = 2.4 Hz, 8H), 2.89 (br s, 1H), 2.71 |
| | | | | (br d, J = 4.4 Hz, 2H), 2.60 (br s, 2H), 2.04-1.97 (m, 1H), |
| | | | | 1.31-1.26 (m, 2H), 1.04-0.99 (m, 2H) |
| I-420 | AQ | BC | 729.5 | 10.80 (s, 1H), 9.17 (d, J = 8.8 Hz, 1H), 9.08-9.05 (m, 1H), |
| | | | | 8.48-8.43 (m, 1H), 8.17 (d, J = 8.8 Hz, 1H), 8.12-8.05 (m, |
| | | | | 2H), 7.96 (d, J = 8.8 Hz, 1H), 7.17-7.10 (m, 2H), 7.00 (d, J = |
| | | | | 9.2 Hz, 1H), 6.86-6.78 (m, 2H), 6.59 (d, J = 7.2 Hz, 1H), |
| | | | | 3.76 (dd, J = 4.8, 10.8 Hz, 1H), 3.71-3.59 (m, 8H), 3.47 (s, |
| | | | | 2H), 3.30 (s, 1H), 2.71-2.63 (m, 4H), 2.23 (d, J = 7.2 Hz, |
| | | | | 3H), 2.20-2.14 (m, 1H), 2.03 (td, J = 4.8, 8.2 Hz, 1H), 1.83 |
| | | | | (d, J = 11.6 Hz, 2H), 1.74 (dd, J = 5.6, 8.6 Hz, 1H), 1.28- |
| | | | | 1.16 (m, 6H) |
| I-421 | AQ | BD | 729.5 | 10.76 (s, 1H), 9.16 (br d, J = 8.8 Hz, 1H), 9.05 (s, 1H), 8.45 |
| | | | | (br d, J = 8.8 Hz, 1H), 8.18-8.15 (m, 1H), 8.11-8.04 (m, |
| | | | | 2H), 7.96 (d, J = 9.2 Hz, 1H), 7.12 (br s, 1H), 7.05-6.97 (m, |
| | | | | 3H), 6.89 (br d, J = 7.6 Hz, 2H), 3.63-3.63 (m, 1H), 3.77- |
| | | | | 3.54 (m, 9H), 3.47 (br s, 3H), 2.73-2.56 (m, 4H), 2.23 (br d, |
| | | | | J = 6.4 Hz, 2H), 2.18-2.07 (m, 1H), 2.06-1.96 (m, 1H), |
| | | | | 1.82 (br d, J = 12.0 Hz, 2H), 1.70 (br d, J = 9.6 Hz, 1H), 1.30- |
| | | | | 1.15 (m, 6H) |
| I-422 | AS | G | 772.5 | 11.49 (s, 1H), 11.00-11.08 (m, 1H), 8.55 (d, J = 5.6 Hz, 1H), |
| | | | | 7.96 (s, 1H), 7.81 (t, J = 9.2 Hz, 1H), 7.59 (dd, J = 5.2, 1.63 |
| | | | | Hz, 1H), 7.14 (s, 1H), 6.95 (s, 1H), 6.92 (s, 1H), 6.88-6.90 |
| | | | | (m, 1H), 6.84 (d, J = 2.0 Hz, 1H), 6.32 (d, J = 2.0 Hz, 1H), |
| | | | | 6.12 (dd, J = 8.4, 2.0 Hz, 1H), 5.27 (dd, J = 13.2, 5.2 Hz, 1H), |
| | | | | 3.95 (t, J = 7.2 Hz, 2H), 3.42-3.48 (m, 3H), 3.29 (s, 9H), |
| | | | | 3.17 (d, J = 2.4 Hz, 2H), 2.82-3.04 (m, 3H), 2.64 (br d, J = |
| | | | | 7.6 Hz, 3H), 1.90-2.00 (m, 3H), 1.76-1.85 (m, 4H), 1.68- |
| | | | | 1.75 (m, 2H), 1.06 (t, J = 7.2 Hz, 1H) |
| I-423 | AR | G | 718 | 11.98 (br s, 1H), 11.11-11.00 (m, 1H), 8.54 (d, J = 5.2 Hz, |
| | | | | 1H), 7.90 (s, 1H), 7.84 (t, J = 9.2 Hz, 1H), 7.51 (dd, J = 1.6, |
| | | | | 5.2 Hz, 1H), 7.06 (br s, 1H), 6.99 (d, J = 2.4 Hz, 1H), 6.93- |
| | | | | 6.82 (m, 3H), 6.33 (d, J = 2.0 Hz, 1H), 6.12 (dd, J = 2.0, 8.4 |
| | | | | Hz, 1H), 5.27 (dd, J = 5.2, 12.8 Hz, 1H), 3.95 (t, J = 7.2 Hz, |
| | | | | 2H), 3.54-3.39 (m, 6H), 3.29 (s, 3H), 3.00-2.92 (m, 1H), |
| | | | | 2.91-2.83 (m, 3H), 2.64 (br d, J = 7.2 Hz, 4H), 2.54 (br s, |
| | | | | 6H), 2.03-1.93 (m, 1H) |
| I-424 | AS | W | 800.6 | 11.49 (br s, 1H), 11.06 (br s, 1H), 8.55 (d, J = 5.2 Hz, 1H), |
| | | | | 7.96 (s, 1H), 7.81 (br t, J = 8.8 Hz, 1H), 7.59 (br d, J = 5.2 |
| | | | | Hz, 1H), 7.15 (br s, 1H), 6.95 (br s, 1H), 6.90 (br dd, J = 14.8, |
| | | | | 4.82 Hz, 2H), 6.83 (s, 1H), 6.65 (br d, J = 8.8 Hz, 1H), 5.29 |
| | | | | (br dd, J = 12.8, 4.94 Hz, 1H), 3.60 (br d, J = 10.8 Hz, 2H), |
| | | | | 3.30 (br s, 6H), 3.17 (br s, 2H), 2.85-2.94 (m, 1H), 2.58- |
| | | | | 2.75 (m, 6H), 2.25 (br d, J = 6.4 Hz, 2H), 1.89-2.05 (m, 4H), |
| | | | | 1.74-1.88 (m, 8H), 1.61-1.74 (m, 4H), 1.23-1.34 (m, 2H) |
| I-425 | AT | G | 744.3 | 11.21 (s, 1H), 11.06 (s, 1H), 8.54 (d, J = 5.2 Hz, 1H), 8.24 (s, |
| | | | | 1H), 7.90 (s, 1H), 7.80 (t, J = 9.2 Hz, 1H), 7.54 (dd, J = 1.6, |
| | | | | 5.2 Hz, 1H), 7.14 (s, 1H), 6.99 (s, 1H), 6.93-6.90 (m, 1H), |
| | | | | 6.88 (br s, 1H), 6.33 (d, J = 2.0 Hz, 1H), 6.12 (dd, J = 2.0, 8.4 |
| | | | | Hz, 1H), 5.28 (dd, J = 5.2, 12.8 Hz, 1H), 3.95 (t, J = 7.2 Hz, |
| | | | | 2H), 3.49-3.42 (m, 4H), 3.29 (s, 3H), 3.26 (br d, J = 2.4 Hz, |
| | | | | 4H), 2.99-2.94 (m, 1H), 2.92-2.84 (m, 1H), 2.70-2.66 (m, |
| | | | | 1H), 2.65 (br s, 1H), 2.63 (br s, 2H), 2.59 (br s, 1H), 2.55 (s, |
| | | | | 2H), 2.01-1.95 (m, 1H), 1.30-1.24 (m, 2H), 1.01-0.96 (m, |
| | | | | 2H) |
| I-426 | H | G | 727.4 | 11.33 (br s, 1H), 11.04 (br s, 1H), 8.64 (d, J = 5.2 Hz, 1H), |
| | | | | 8.43 (d, J = 8.8 Hz, 2H), 7.57 (d, J = 5.2 Hz, 1H), 7.30 (s, |
| | | | | 1H), 7.22 (s, 1H), 7.05 (d, J = 9.2 Hz, 2H), 6.91 (d, J = 8.4 |
| | | | | Hz, 1H), 6.33 (d, J = 2.0 Hz, 1H), 6.12 (dd, J = 8.4, 2.0 Hz, |
| | | | | 1H), 5.21-5.33 (m, 1H), 3.95 (t, J = 7.6 Hz, 2H), 3.46 (br t, J = |
| | | | | 6.4 Hz, 2H), 3.30 (s, 5H), 3.27-3.29 (m, 5H), 2.84-3.00 |
| | | | | (m, 2H), 2.65 (br d, J = 7.2 Hz, 2H), 2.56 (br s, 4H), 1.93- |
| | | | | 2.03 (m, 1H), 1.36-1.45 (m, 2H), 0.96-1.06 (m, 2H). |
| I-427 | AV | W | 729.5 | 12.08-12.01 (m, 1H), 11.06 (br s, 1H), 8.62 (d, J = 5.2 Hz, |
| | | | | 1H), 8.45 (d, J = 8.8 Hz, 2H), 7.55 (d, J = 5.2 Hz, 1H), 7.26 |
| | | | | (d, J = 2.0 Hz, 1H), 7.13 (br s, 1H), 7.04 (d, J = 9.2 Hz, 2H), |
| | | | | 6.93 (d, J = 8.8 Hz, 1H), 6.83 (d, J = 2.0 Hz, 1H), 6.64 (dd, J = |
| | | | | 2.0, 8.6 Hz, 1H), 5.29 (dd, J = 5.M285 Hz, 1H), 1.38-1.21 |
| | | | | (m, 3H) |
| I-428 | H | W | 755.6 | 11.33 (br s, 1H), 11.08-11.07 (m, 1H), 11.06 (br s, 1H), 8.63 |
| | | | | (d, J = 5.6 Hz, 1H), 8.42 (d, J = 8.8 Hz, 2H), 7.56 (d, J = 5.2 |
| | | | | Hz, 1H), 7.29 (d, J = 1.6 Hz, 1H), 7.22 (br s, 1H), 7.04 (br d, |
| | | | | J = 9.2 Hz, 2H), 6.93 (d, J = 8.4 Hz, 1H), 6.83 (d, J = 2.0 Hz, |
| | | | | 1H), 6.64 (dd, J = 2.0, 8.6 Hz, 1H), 5.29 (br dd, J = 5.2, 12.9 |
| | | | | Hz, 1H), 3.60 (br d, J = 12.0 Hz, 2H), 3.31 (s, 3H), 3.27 (d, J = |
| | | | | 2.4 Hz, 2H), 2.98-2.81 (m, 2H), 2.74-2.68 (m, 2H), 2.66- |
| | | | | 2.59 (m, 7H), 2.24 (br d, J = 6.8 Hz, 2H), 2.03-1.94 (m, 1H), |
| | | | | 1.83 (br d, J = 11.6 Hz, 2H), 1.74-1.67 (m, 1H), 1.42-1.38 |
| | | | | (m, 2H), 1.37-1.20 (m, 3H), 1.02-0.98 (m, 2H) |
| I-430^{b} | V | S | 715.6 | 11.13 (d, J = 1.2 Hz, 1H), 11.08 (s, 1H), 8.47 (d, J = 5.2 Hz, |
| | | | | 1H), 8.14 (s, 1H), 8.08 (s, 1H), 8.05 (d, J = 8.8 Hz, 2H), 7.48 |
| | | | | (dd, J = 1.6, 5.2 Hz, 1H), 7.12 (d, J = 2.4 Hz, 2H), 7.05 (d, J = |
| | | | | 9.2 Hz, 2H), 6.93 (d, J = 8.8 Hz, 1H), 6.71 (d, J = 8.8 Hz, |
| | | | | 1H), 5.33 (dd, J = 5.2, 12.4 Hz, 1H), 3.80 (s, 3H), 3.60 (s, |
| | | | | 3H), 3.31-3.27 (m, 7H), 3.14-3.09 (m, 2H), 2.93-2.85 (m, |
| | | | | 1H), 2.70 (d, J = 4.0 Hz, 4H), 2.64 (s, 1H), 2.59 (d, J = 8.0 |
| | | | | Hz, 2H), 2.03-1.94 (m, 1H), 1.32-1.26 (m, 2H), 1.04-0.98 |
| | | | | (m, 2H) |
| I-432 | BO | W | 842.4 | 11.06 (s, 1H), 9.19 (d, J = 8.8 Hz, 1H), 8.12 (d, J = 8.8 Hz, |
| | | | | 1H), 8.08 (br d, J = 4.0 Hz, 1H), 7.96 (d, J = 8.8 Hz, 1H), |
| | | | | 7.80 (d, J = 8.8 Hz, 1H), 7.16-7.12 (m, 2H), 6.93 (d, J = 8.4 |
| | | | | Hz, 1H), 6.85 (s, 1H), 6.83 (d, J = 1.6 Hz, 1H), 6.67-6.61 |
| | | | | (m, 1H), 5.33-5.25 (m, 1H), 3.83 (s, 3H), 3.60 (br d, J = 11.6 |
| | | | | Hz, 3H), 3.47 (br d, J = 4.0 Hz, 2H), 3.31-3.31 (m, 3H), 3.08 |
| | | | | (br s, 3H), 2.94-2.84 (m, 1H), 2.65-2.61 (m, 4H), 2.59- |
| | | | | 2.55 (m, 5H), 2.37 (s, 3H), 2.26 (br d, J = 6.8 Hz, 2H), 2.03- |
| | | | | 1.96 (m, 1H), 1.87-1.81 (m, 2H), 1.72-1.64 (m, 1H), 1.33- |
| | | | | 1.25 (m, 2H), 1.19 (d, J = 6.8 Hz, 3H) |
| I-437^{b} | J | BG | 705.5 | 10.30 (s, 1H), 9.17 (br d, J = 9.2 Hz, 1H), 9.06 (d, J = 2.4 Hz, |
| | | | | 1H), 8.46 (dd, J = 9.2, 2.25 Hz, 1H), 8.17 (br d, J = 9.2 Hz, |
| | | | | 1H), 8.05-8.11 (m, 2H), 7.96 (d, J = 9.2 Hz, 1H), 7.23 (br d, |
| | | | | J = 8.8 Hz, 2H), 7.10-7.15 (m, 1H), 6.94-7.02 (m, 3H), |
| | | | | 4.02 (br t, J = 6.4 Hz, 2H), 3.72 (br t, J = 6.4 Hz, 3H), 3.63 |
| | | | | (br s, 6H), 3.47 (br s, 2H), 2.70 (br t, J = 6.8 Hz, 3H), 2.40 (br |
| | | | | t, J = 6.8 Hz, 3H), 1.75-1.80 (m, 2H), 1.61-1.67 (m, 2H), |
| | | | | 1.20 (br d, J = 6.8 Hz, 3H) |
| I-438^{b} | J | BH | 730.5 | 10.26 (s, 1H), 9.17 (br d, J = 9.2 Hz, 1H), 9.06 (d, J = 2.4 Hz, |
| | | | | 1H), 8.46 (dd, J = 8.8, 2.4 Hz, 1H), 8.17 (br d, J = 8.8 Hz, |
| | | | | 1H), 8.06-8.11 (m, 2H), 7.96 (d, J = 8.8 Hz, 1H), 7.14 (br d, |
| | | | | J = 8.8 Hz, 3H), 7.00 (br d, J = 8.8 Hz, 1H), 6.94 (br d, J = |
| | | | | 9.2 Hz, 2H), 3.68-3.72 (m, 5H), 3.64 (br s, 4H), 3.48 (br s, |
| | | | | 2H), 2.69 (br t, J = 6.4 Hz, 6H), 2.22-2.26 (m, 2H), 1.80- |
| | | | | 1.90 (m, 3H), 1.71-1.77 (m, 1H), 1.23-1.30 (m, 3H), 1.20 |
| | | | | (d, J = 6.8 Hz, 3H) |
| I-439 | BP | BQ | 734.1 | 11.06 (s, 1H), 8.86 (d, J = 9.6 Hz, 1H), 8.15 (s, 1H), 7.98 (br |
| | | | | d, J = 4.4 Hz, 1H), 7.87 (d, J = 8.8 Hz, 1H), 7.58 (d, J = 8.8 |
| | | | | Hz, 1H), 7.25 (d, J = 9.6 Hz, 1H), 6.91 (br d, J = 8.4 Hz, 2H), |
| | | | | 6.32 (d, J = 2.0 Hz, 1H), 6.12 (dd, J = 2.0, 8.4 Hz, 1H), 5.27 |
| | | | | (dd, J = 5.2, 12.8 Hz, 1H), 4.53 (br d, J = 12.8 Hz, 2H), 3.87 |
| | | | | (s, 4H), 3.59 (br s, 4H), 3.42 (br s, 2H), 3.29 (s, 3H), 2.96- |
| | | | | 2.83 (m, 4H), 2.73-2.66 (m, 1H), 2.66-2.62 (m, 1H), 2.59 |
| | | | | (br d, J = 3.6 Hz, 1H), 2.55 (br s, 1H), 2.54-2.53 (m, 1H), |
| | | | | 2.02-1.94 (m, 1H), 1.82-1.76 (m, 2H), 1.66 (br s, 1H), 1.18 |
| | | | | (d, J = 6.8 Hz, 3H) |
| I-440 | P | R | 748.4 | 11.07 (d, J = 2.4 Hz, 1H), 8.86 (d, J = 9.2 Hz, 1H), 7.98 (d, J = |
| | | | | 4.0 Hz, 1H), 7.87 (d, J = 8.8 Hz, 1H), 7.58 (d, J = 8.8 Hz, |
| | | | | 1H), 7.26 (d, J = 9.6 Hz, 1H), 6.93 (d, J = 8.4 Hz, 2H), 6.84 |
| | | | | (d, J = 2.0 Hz, 1H), 6.67-6.59 (m, 1H), 5.34-5.25 (m, 1H), |
| | | | | 4.34-4.24 (m, 2H), 3.61-3.54 (m, 1H), 3.43-3.41 (m, 2H), |
| | | | | 3.30 (s, 3H), 3.11 (d, J = 10.8 Hz, 2H), 3.04 (s, 4H), 2.99 (s, |
| | | | | 4H), 2.91-2.84 (m, 1H), 2.70-2.65 (m, 2H), 2.59 (s, 1H), |
| | | | | 2.02-1.95 (m, 1H), 1.82-1.73 (m, 6H), 1.23 (d, J = 8.4 Hz, |
| | | | | 2H), 1.18 (d, J = 6.8 Hz, 3H) |
| I-441 | BJ | BI | 748.4 | 11.18-11.01 (m, 1H), 8.86 (d, J = 9.6 Hz, 1H), 8.23-8.16 |
| | | | | (m, 1H), 7.99-7.94 (m, 1H), 7.89-7.84 (m, 1H), 7.61-7.56 |
| | | | | (m, 1H), 7.31-7.25 (m, 1H), 7.00-6.95 (m, 1H), 6.93-6.89 |
| | | | | (m, 2H), 6.89-6.85 (m, 1H), 5.40-5.30 (m, 1H), 3.68 (br s, |
| | | | | 4H), 3.61 (s, 3H), 3.15-3.03 (m, 8H), 2.87 (br d, J = 11.6 |
| | | | | Hz, 1H), 2.75-2.63 (m, 4H), 2.29-2.19 (m, 1H), 2.03-1.97 |
| | | | | (m, 1H), 1.77 (br s, 6H), 1.43-1.33 (m, 2H), 1.17 (d, J = 6.6 |
| | | | | Hz, 3H) |
| I-443 | AL | BB | 748.4 | 11.08 (s, 1H), 8.88 (d, J = 9.6 Hz, 1H), 8.00-7.94 (m, 1H), |
| | | | | 7.88 (d, J = 8.8 Hz, 1H), 7.60 (d, J = 9.2 Hz, 1H), 7.26 (d, J = |
| | | | | 9.6 Hz, 1H), 6.99-6.85 (m, 4H), 5.39-5.30 (m, 1H), 3.69 (s, |
| | | | | 4H), 3.63 (s, 3H), 3.57 (d, J = 10.8 Hz, 1H), 3.45-3.40 (m, |
| | | | | 2H), 3.01-2.84 (m, 3H), 2.80-2.73 (m, 2H), 2.72-2.57 (m, |
| | | | | 3H), 2.40 (s, 4H), 2.04-1.90 (m, 2H), 1.79-1.59 (m, 6H), |
| | | | | 1.18 (d, J = 6.8 Hz, 3H) |
| I-444 | AI | AK | 750.4 | 11.04 (s, 1H), 8.84 (d, J = 9.5 Hz, 1H), 7.95-7.87 (m, 1H), |
| | | | | 7.87-7.77 (m, 1H), 7.65-7.50 (m, 1H), 7.12-7.03 (m, 1H), |
| | | | | 6.90-6.80 (m, 2H), 6.73 (d, J = 1.9 Hz, 1H), 6.42 (br dd, J = |
| | | | | 2.5, 8.6 Hz, 1H), 5.31-5.18 (m, 1H), 3.91-3.82 (m, 2H), |
| | | | | 3.82-3.73 (m, 2H), 3.58-3.50 (m, 1H), 3.43-3.37 (m, 4H), |
| | | | | 3.28 (s, 3H), 2.95-2.85 (m, 1H), 2.78 (br t, J = 4.8 Hz, 2H), |
| | | | | 2.72-2.62 (m, 2H), 2.61-2.53 (m, 4H), 2.39-2.32 (m, 2H), |
| | | | | 1.98 (td, J = 5.0, 9.9 Hz, 1H), 1.91-1.85 (m, 2H), 1.68-1.60 |
| | | | | (m, 2H), 1.42-1.32 (m, 2H), 1.32-1.25 (m, 1H), 1.25-1.18 |
| | | | | (m, 2H), 1.14 (dd, J = 3.8, 6.6 Hz, 3H) |
| I-445 | AL | AK | 736.3 | 11.16-10.83 (m, 1H), 8.88 (d, J = 9.4 Hz, 1H), 8.02-7.92 |
| | | | | (m, 1H), 7.88 (d, J = 8.9 Hz, 1H), 7.60 (d, J = 8.9 Hz, 1H), |
| | | | | 7.25 (d, J = 9.5 Hz, 1H), 6.96-6.89 (m, 2H), 6.82 (d, J = 1.8 |
| | | | | Hz, 1H), 6.66-6.60 (m, 1H), 5.33-5.24 (m, 1H), 3.73-3.64 |
| | | | | (m, 4H), 3.57 (br d, J = 10.8 Hz, 3H), 3.42 (br d, J = 3.6 Hz, |
| | | | | 2H), 3.30 (s, 3H), 2.95-2.84 (m, 1H), 2.71-2.65 (m, 1H), |
| | | | | 2.62 (br d, J = 5.6 Hz, 2H), 2.58 (br s, 1H), 2.54 (s, 4H), 2.44- |
| | | | | 2.38 (m, 2H), 2.01-1.95 (m, 1H), 1.80 (br d, J = 12.1 Hz, |
| | | | | 2H), 1.52-1.42 (m, 3H), 1.37-1.29 (m, 2H), 1.18 (d, J = 6.8 |
| | | | | Hz, 3H) |
| I-452 | BE | AF | 748.5 | 11.13-11.01 (m, 1H), 8.87 (d, J = 9.2 Hz, 1H), 7.96 (d, J = |
| | | | | 4.0 Hz, 1H), 7.84 (d, J = 8.8 Hz, 1H), 7.56 (d, J = 8.8 Hz, |
| | | | | 1H), 7.05-6.93 (m, 2H), 6.92-6.88 (m, 2H), 6.86 (d, J = 7.6 |
| | | | | Hz, 1H), 5.35 (dd, J = 5.2, 12.8 Hz, 1H), 4.96-4.43 (m, 1H), |
| | | | | 3.90-3.77 (m, 1H), 3.63 (s, 3H), 3.58 (d, J = 4.4 Hz, 1H), |
| | | | | 3.48 (dt, J = 3.6, 6.4 Hz, 1H), 3.43 (s, 2H), 3.17-3.07 (m, |
| | | | | 2H), 3.03-2.95 (m, 2H), 2.93-2.84 (m, 2H), 2.76-2.61 (m, |
| | | | | 4H), 2.08-1.96 (m, 2H), 1.94-1.74 (m, 5H), 1.71-1.63 (m, |
| | | | | 1H), 1.59-1.49 (m, 1H), 1.39-1.27 (m, 2H), 1.18 (d, J = 6.8 |
| | | | | Hz, 3H) |
| I-464 | AD | G | 700.1 | 12.36-11.98 (m, 1H), 11.06 (s, 1H), 8.51 (d, J = 5.6 Hz, 1H), |
| | | | | 8.22 (br s, 1H), 8.12 (br d, J = 8.8 Hz, 2H), 7.58 (br d, J = 1.2 |
| | | | | Hz, 1H), 7.23 (br s, 1H), 7.18-7.09 (m, 3H), 6.94 (d, J = 8.4 |
| | | | | Hz, 1H), 6.34 (d, J = 2.0 Hz, 1H), 6.14 (dd, J = 2.0, 8.4 Hz, |
| | | | | 1H), 5.29 (br dd, J = 5.2, 12.8 Hz, 1H), 4.09-4.01 (m, 3H), |
| | | | | 3.60 (br t, J = 6.4 Hz, 3H), 3.52 (br s, 3H), 3.44 (dt, J = 2.8, |
| | | | | 6.8 Hz, 3H), 3.30 (s, 3H), 3.27-3.18 (m, 4H), 2.96-2.84 (m, |
| | | | | 4H), 2.65 (br d, J = 7.2 Hz, 1H), 2.60 (br s, 1H), 2.03-1.93 |
| | | | | (m, 1H) |
| I-465 | AH | AF | 675.5 | 11.16-11.02 (m, 1H), 8.89 (d, J = 2.4 Hz, 1H), 8.62 (s, 1H), |
| | | | | 8.53 (d, J = 5.2 Hz, 1H), 8.24 (dd, J = 2.4, 9.2 Hz, 1H), 7.97 |
| | | | | (s, 1H), 7.56 (s, 1H), 7.30 (dd, J = 1.6, 5.2 Hz, 1H), 7.00- |
| | | | | 6.91 (m, 4H), 5.37 (dd, J = 5.6, 12.4 Hz, 1H), 3.92-3.83 (m, |
| | | | | 1H), 3.62 (s, 4H), 3.61 (s, 3H), 3.15-3.09 (m, 2H), 2.94- |
| | | | | 2.84 (m, 1H), 2.76-2.68 (m, 1H), 2.65-2.59 (m, 7H), 2.43- |
| | | | | 2.37 (m, 1H), 2.32-2.24 (m, 1H), 2.00 (td, J = 5.2, 10.4 Hz, |
| | | | | 1H), 1.28 (d, J = 6.4 Hz, 3H) |
| I-466 | AD | AE | 658.4 | 12.03-11.89 (m, 1H), 10.91-10.62 (m, 1H), 8.47 (d, J = 5.2 |
| | | | | Hz, 1H), 8.09 (s, 1H), 8.05 (d, J = 8.8 Hz, 2H), 7.48-7.43 |
| | | | | (m, 1H), 7.11 (s, 1H), 7.06-7.01 (m, 5H), 6.89 (d, J = 8.8 |
| | | | | Hz, 2H), 3.76-3.70 (m, 1H), 3.70-3.64 (m, 2H), 3.45-3.41 |
| | | | | (m, 2H), 3.36-3.33 (m, 2H), 3.25 (br s, 4H), 2.87 (t, J = 6.8 |
| | | | | Hz, 2H), 2.72-2.56 (m, 4H), 2.49-2.42 (m, 2H), 2.23 (br d, |
| | | | | J = 7.2 Hz, 2H), 2.16-2.07 (m, 1H), 2.00 (qd, J = 5.2, 13.2 |
| | | | | Hz, 1H), 1.82 (br d, J = 11.6 Hz, 2H), 1.75-1.66 (m, 1H), |
| | | | | 1.23 (br d, J = 9.0 Hz, 2H) |
| I-467 | V | AE | 684.4 | 11.17 (s, 1H), 10.71 (br d, J = 4.4 Hz, 1H), 8.50-8.44 (m, |
| | | | | 1H), 8.07 (s, 1H), 8.04 (br d, J = 8.6 Hz, 2H), 7.47 (br d, J = |
| | | | | 5.8 Hz, 1H), 7.11 (s, 2H), 7.03 (br d, J = 8.4 Hz, 4H), 6.89 (br |
| | | | | d, J = 8.4 Hz, 2H), 3.75-3.71 (m, 1H), 3.70-3.65 (m, 2H), |
| | | | | 3.31-3.22 (m, 10H), 2.71-2.57 (m, 6H), 2.23 (br d, J = 6.6 |
| | | | | Hz, 2H), 2.16-2.09 (m, 1H), 2.01 (br dd, J = 4.9, 13.4 Hz, |
| | | | | 1H), 1.85-1.79 (m, 2H), 1.74-1.68 (m, 1H), 1.30-1.27 (m, |
| | | | | 2H), 1.01 (br s, 2H) |
| I-468 | AD | AP | 686.3 | 11.98 (br s, 1H), 11.25-10.92 (m, 1H), 8.48 (d, J = 5.2 Hz, |
| | | | | 1H), 8.10 (s, 1H), 8.06 (br d, J = 8.8 Hz, 2H), 7.46 (dd, J = |
| | | | | 1.6, 5.2 Hz, 1H), 7.12 (d, J = 2.0 Hz, 1H), 7.08-7.03 (m, |
| | | | | 3H), 7.01-6.95 (m, 1H), 6.76 (d, J = 8.0 Hz, 1H), 6.71 (d, J = |
| | | | | 8.0 Hz, 1H), 5.34 (br dd, J = 5.2, 12.8 Hz, 1H), 4.01-3.91 |
| | | | | (m, 2H), 3.72 (br t, J = 5.2 Hz, 2H), 3.60 (s, 3H), 3.47-3.41 |
| | | | | (m, 3H), 3.29 (br d, J = 4.4 Hz, 6H), 2.88 (br t, J = 6.8 Hz, |
| | | | | 3H), 2.78-2.59 (m, 2H), 2.53 (br s, 2H), 2.05-1.96 (m, 1H) |
| I-469 | H | X | 686.3 | 8.60 (d, J = 5.4 Hz, 1H), 8.38 (d, J = 9.0 Hz, 2H), 7.51 (d, J = |
| | | | | 5.4 Hz, 1H), 7.27 (s, 1H), 7.03 (br d, J = 9.2 Hz, 2H), 7.00- |
| | | | | 6.96 (m, 2H), 6.95-6.91 (m, 1H), 5.33-5.24 (m, 1H), 3.58 |
| | | | | (s, 3H), 3.30 (br s, 4H), 3.28 (br d, J = 2.8 Hz, 2H), 3.13- |
| | | | | 3.06 (m, 2H), 2.89-2.81 (m, 1H), 2.68-2.60 (m, 8H), 2.03- |
| | | | | 1.97 (m, 1H), 1.39-1.33 (m, 2H), 1.04-0.99 (m, 2H) |
| I-470 | AZ | X | 687.2 | 11.32-11.27 (m, 1H), 9.36 (d, J = 2.4 Hz, 1H), 8.63 (d, J = |
| | | | | 5.2 Hz, 1H), 8.57 (dd, J = 2.4, 9.2 Hz, 1H), 7.60 (d, J = 5.2 |
| | | | | Hz, 1H), 7.32 (s, 1H), 7.22 (s, 1H), 7.02-6.91 (m, 5H), 5.42- |
| | | | | 5.35 (m, 1H), 3.68 (s, 4H), 3.62 (s, 3H), 3.28 (d, J = 2.4 Hz, |
| | | | | 2H), 3.16-3.11 (m, 2H), 2.96-2.83 (m, 2H), 2.69-2.67 (m, |
| | | | | 2H), 2.64-2.62 (m, 5H), 2.04-1.97 (m, 1H), 1.43-1.40 (m, |
| | | | | 2H), 1.04-1.00 (m, 2H) |
| I-471 | BM | X | 699.5 | 11.71 (s, 1H), 11.11 (s, 1H), 8.50 (d, J = 5.2 Hz, 1H), 8.19 (s, |
| | | | | 1H), 8.08 (d, J = 8.8 Hz, 2H), 7.56 (dd, J = 5.2, 1.6 Hz, 1H), |
| | | | | 7.19 (br s, 1H), 7.06-7.11 (m, 3H), 6.99 (br d, J = 5.6 Hz, |
| | | | | 2H), 6.94-6.97 (m, 1H), 5.38 (br dd, J = 12.4, 5.57 Hz, 1H), |
| | | | | 3.62 (s, 3H), 3.47 (br d, J = 2.4 Hz, 2H), 3.30 (br d, J = 4.0 |
| | | | | Hz, 4H), 3.11-3.16 (m, 2H), 2.85-2.93 (m, 1H), 2.72-2.78 |
| | | | | (m, 1H), 2.63-2.70 (m, 8H), 1.94-2.14 (m, 6H) |
| I-472 | AD | X | 659.3 | 11.96 (br s, 1H), 11.09 (s, 1H), 8.50-8.45 (m, 1H), 8.15 (s, |
| | | | | 1H), 8.10 (s, 1H), 8.07-8.03 (m, 2H), 7.45 (dd, J = 1.2, 5.2 |
| | | | | Hz, 1H), 7.11 (d, J = 2.4 Hz, 1H), 7.05-7.05 (m, 1H), 7.07- |
| | | | | 7.04 (m, 2H), 6.98 (br d, J = 5.6 Hz, 1H), 6.96-6.92 (m, 1H), |
| | | | | 5.37 (br dd, J = 5.6, 12.4 Hz, 1H), 3.47-3.39 (m, 3H), 3.29 |
| | | | | (br s, 5H), 3.17-3.09 (m, 3H), 2.95-2.84 (m, 3H), 2.77- |
| | | | | 2.60 (m, 8H), 2.04-1.97 (m, 1H) |

Footnotes:
^{a}The reductive amination was run anywhere from 0-25° C. for 2-16 hrs under standard techniques. The products were purified under standard techniques such as prep-HPLC and chromatography under a variety of solvent conditions.
^{b}The product of the coupling was further deprotected with TfOH in TFA at 50-70° C. for 0.5-4 hr. The final product was purified by prep-HPLC.
^{C}NMR reported in CD₃OD-d4 as the solvent.

### Example 2 (Method 2): Synthesis of 3-(4-(2-(4-(5-((R)-10-methyl-8-oxo-9,10,11,12-tetrahydro-8H-[1,4]diazepino[5′,6′:4,5]thieno[3,2-f]quinolin-3-yl)pyridin-2-yl)piperazin-1-yl)ethoxy)phenyl)piperidine-2,6-dione (I-473)

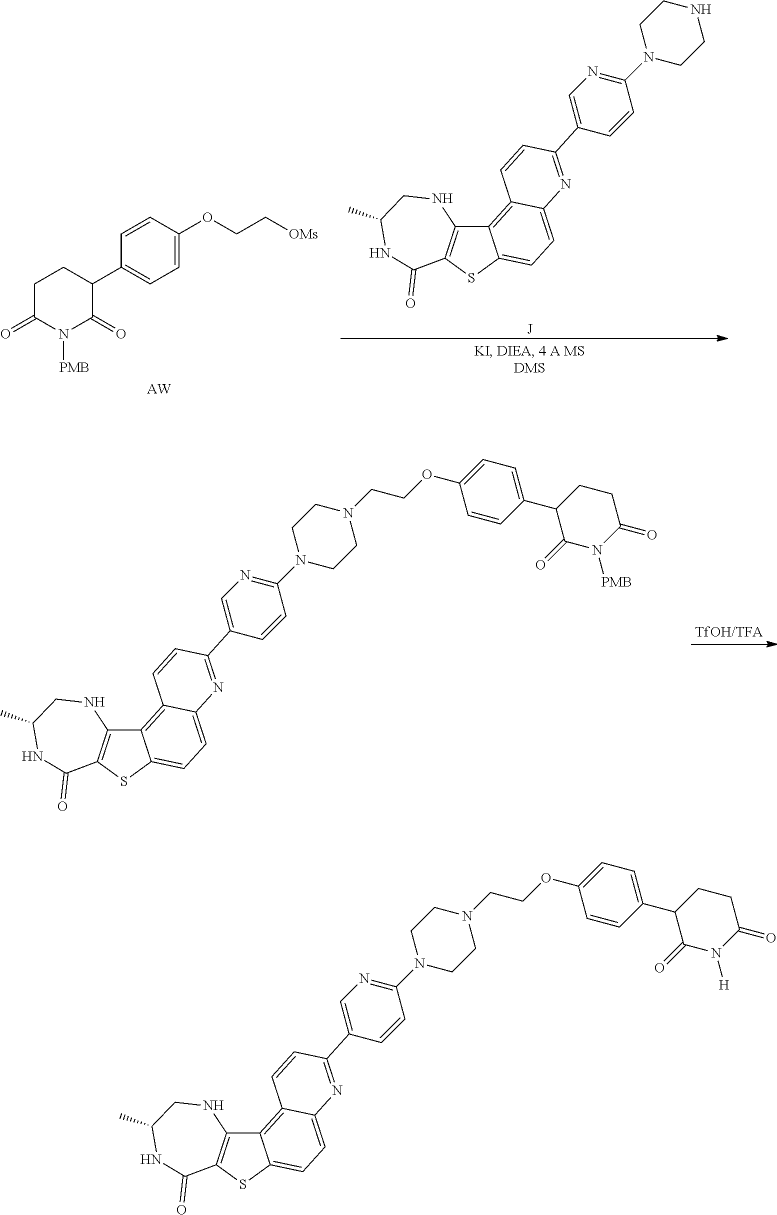

Step 1—1-(4-methoxybenzyl)-3-(4-(2-(4-(5-((R)-10-methyl-8-oxo-9,10,11,12-tetrahydro-8H-[1,4]diazepino[5′,6′:4,5]thieno[3,2-f]quinolin-3-yl)pyridin-2-yl)piperazin-1-yl)ethoxy)phenyl)piperidine-2,6-dione. To a solution of 2-(4-(1-(4-methoxybenzyl)-2,6-dioxopiperidin-3-yl)phenoxy)ethyl methanesulfonate (45 mg, 100 umol, Intermediate AW) in DMF (2 mL) was added (R)-10-methyl-3-(6-(piperazin-1-yl)pyridin-3-yl)-9,10,11,12-tetrahydro-8H-[1,4]diazepino[5′,6′:4,5]thieno[3,2-f]quinolin-8-one (44.7 mg, 100 umol, Intermediate J), 4 Å molecular sieves (5 mg), DIEA (38.9 mg, 301 umol) and KI (1.67 mg, 10 umol). The mixture was stirred at 70° C. for 12 hrs. On completion, the reaction mixture was filtered and concentrated under reduced pressure to give a residue. The crude product was purified by reversed-phase HPLC (0.1% FA condition) to give the title compound (25 mg, 31% yield) as a white oil. LC-MS (ESI⁺) m/z 796.3 (M+H)⁺.

Step 2—3-(4-(2-(4-(5-((R)-10-methyl-8-oxo-9,10,11,12-tetrahydro-8H-[1,4]diazepino[5′,6′: 4,5]thieno[3,2-f]quinolin-3-yl)pyridin-2-yl)piperazin-1-yl)ethoxy)phenyl)piperidine-2,6-dione. To a solution of 1-(4-methoxybenzyl)-3-(4-(2-(4-(5-((R)-10-methyl-8-oxo-9,10,11,12-tetrahydro-8H-[1,4]diazepino[5′,6′:4,5]thieno[3,2-f]quinolin-3-yl)pyridin-2-yl)piperazin-1l-yl)ethoxy)phenyl)piperidine-2,6-dione (20 mg, 25.1 umol) in TFA (2 mL) was added TfOH (680 mg, 4.53 mmol) and the mixture was stirred at 60° C. for 3 hr. On completion, the reaction mixture was filtered and concentrated under reduced pressure to give a residue. The residue was purified by reversed-phase HPLC (0.1% FA condition) to give the title compound (9.5 mg) as a yellow solid. ¹H NMR (400 MHz, DMSO-d₆) δ=10.80 (s, 1H), 9.17 (d, J=9.2 Hz, 1H), 9.07 (d, J=2.4 Hz, 1H), 8.46 (dd, J=2.4, 9.2 Hz, 1H), 8.22 (s, 1H), 8.18 (d, J=9.2 Hz, 1H), 8.12-8.05 (i, 2H), 7.96 (d, J=8.8 Hz, 1H), 7.15 (s, 1H), 7.13 (s, 2H), 7.02 (d, J=9.2 Hz, 1H), 6.94 (d, J=8.4 Hz, 2H), 4.14 (t, J=5.6 Hz, 2H), 3.80 (dd, J=5.2, 11.1 Hz, 1H), 3.68-3.61 (i, 5H), 3.47 (s, 2H), 2.78 (t, J=5.6 Hz, 2H), 2.70-2.62 (m, 5H), 2.48-2.45 (m, 1H), 2.20-2.12 (m, 1H), 2.06-1.99 (m, 1H), 1.20 (d, J=6.8 Hz, 3H); LC-MS (ESI) m/z 676.5 (M+H)⁺.

TABLE 3
Compounds synthesized via Method 2 using the corresponding amines and
mesylate/tosylates for the coupling.

| | | | LCMS | |
|---|---|---|---|---|
| | | | (ESI+) | |
| | | Mesylate/ | m/z | |
| I-# | Amine | tosylate | (M + H)⁺ | ¹H NMR (400 MHz, DMSO-d6) |
| I-195 | AQ | CE | 784.6 | 11.13 (s, 1H), 9.51-9.60 (m, 1H), 9.24-9.30 (m, 1H), 8.33 |
| | | | | (d, J = 8.8 Hz, 2H), 8.26 (br d, J = 8.8 Hz, 1H), 8.14-8.21 |
| | | | | (m, 2H), 8.05 (d, J = 8.8 Hz, 1H),7.28 (br d, J = 8.8 Hz, |
| | | | | 2H), 7.21 (br d, J = 5.2 Hz, 1H), 7.00 (d, J =8.4 Hz, 1H), |
| | | | | 6.48 (d, J = 2.0 Hz, 1H), 6.31-6.37 (m, 1H), 5.34 (dd, J = |
| | | | | 12.4, 5.2 Hz, 1H), 4.07-4.17 (m, 2H), 3.72-3.87 (m, H), |
| | | | | 3.67 (br d, J = 3.6 Hz, 2H), 3.61 (br s, 1H), 3.45 (br s, 1H), |
| | | | | 3.37 (s, 3H), 3.25-3.33 (m, 3H), 3.11-3.17 (m, 1H), 2.88- |
| | | | | 2.99 (m, 2H), 2.68-2.77 (m, 2H), 2.61 (s, 3H), 2.33 (br d, |
| | | | | J = 6.4 Hz, 1H), 2.00-2.10 (m, 1H), 1.83-1.93 (m, 1H), |
| | | | | 1.29-1.34 (m, 1H), 1.27 (d, J = 6.8 Hz, 3H) |
| I-214^{b} | AQ | CR | 784.5 | 11.04 (br s, 1H), 9.16 (d, J = 9.2 Hz, 1H), 8.20 (d, J = 8.8 |
| | | | | Hz, 2H), 8.14 (d, J = 9.2 Hz, 1H), 8.10-8.04 (m, 2H), 7.96 |
| | | | | (d, J = 9.2 Hz, 1H), 7.10 (br d, J = 9.2 Hz, 3H), 6.90 (d, J = |
| | | | | 8.4 Hz, 1H), 6.40 (d, J = 1.6 Hz, 1H), 6.24 (dd, J = 1.6, 8.8 |
| | | | | Hz, 1H), 5.27 (dd, J = 5.2, 13.2 Hz, 1H), 3.66-3.58 (m, |
| | | | | 1H), 3.48 (br s, 2H), 3.41 (br t, J = 8.4 Hz, 1H), 3.31 (s, |
| | | | | 7H), 3.25 (br d, J = 8.0 Hz, 1H), 3.05-2.98 (m, 1H), 2.94- |
| | | | | 2.84 (m, 1H), 2.73-2.54 (m, 8H), 2.44-2.43 (m, 1H), 2.43 |
| | | | | (br t, J = 6.4 Hz, 1H), 2.13 (br d, J = 6.4 Hz, 1H), 2.04- |
| | | | | 1.94 (m, 1H), 1.74 (br dd, J = 7.6, 12.4 Hz, 1H), 1.20 (d, J = |
| | | | | 6.4 Hz, 3H) |
| I-474 | AQ | FR | 690.5 | 10.79 (s, 1H), 9.17 (d, J = 9.2 Hz, 1H), 9.07 (d, J = 2.4 Hz, |
| | | | | 1H), 8.49-8.44 (m, 1H), 8.18 (d, J = 9.2 Hz, 1H), 8.15- |
| | | | | 8.08 (m, 1H), 8.06 (d, J = 4.4 Hz, 1H), 7.96 (d, J = 9.2 Hz, |
| | | | | 1H), 7.14 (d, J = 8.8 Hz, 3H), 7.02 (d, J = 9.2 Hz, 1H), 6.91 |
| | | | | (d, J = 8.8 Hz, 2H), 4.04 (t, J = 6.0 Hz, 2H), 3.81-3.77 (m, |
| | | | | 1H), 3.69-3.58 (m, 5H), 3.48 (s, 2H), 2.70-2.57 (m, 6H), |
| | | | | 2.48-2.44 (m, 1H), 2.22-2.12 (m, 1H), 2.04-1.91 (m, |
| | | | | 3H), 1.24 (s, 1H), 1.20 (d, J = 6.8 Hz, 3H) |

^{a}Footnotes: The coupling was run anywhere from 0-100° C. for 12-16 hrs under standard techniques. The products were purified under standard techniques such as prep-HPLC and chromatography under a variety of solvent conditions.
^{b}The product of the coupling was further deprotected with TfOH in TFA at 60° C. for 3 hr.

### Example 3: Synthesis of 3-(3-methyl-5-(2-(5-((R)-10-methyl-8-oxo-9,10,11,12-tetrahydro-8H-[1,4]diazepino[5′,6′:4,5]thieno[3,2-f]quinolin-3-yl)pyridin-2-yl)-2,7-diazaspiro[3.5]nonan-7-yl)-2-oxo-2,3-dihydro-1H-benzo[d]imidazol-1-yl)piperidine-2,6-dione (I-409)

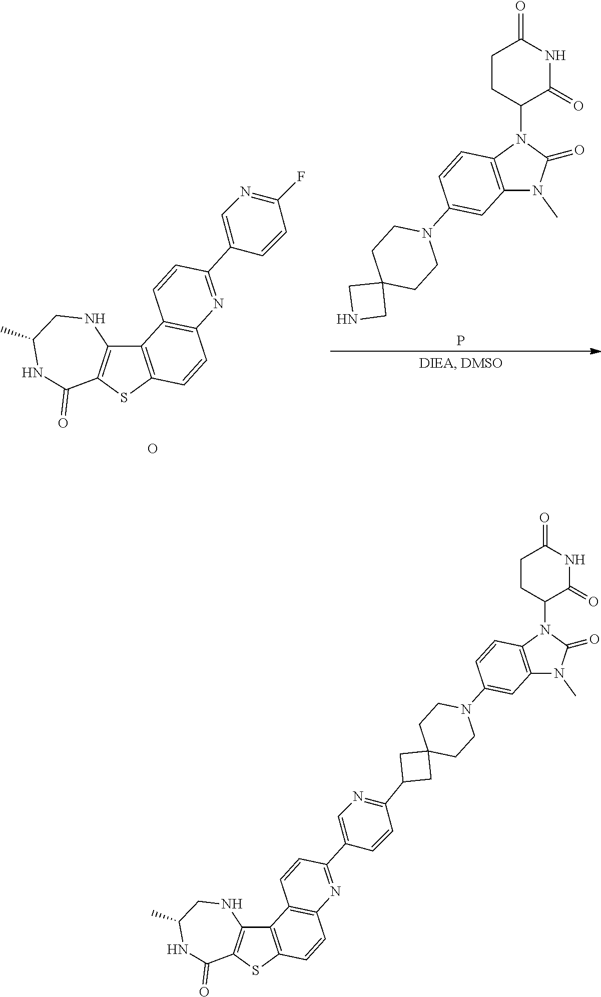

To a solution of (15R)-5-(6-fluoro-3-pyridyl)-15-methyl-11-thia-6,14,17-triazatetracyclo[8.8.0.0^{2,7}.0^{12,18}]octadeca-1(10),2(7),3,5,8,12(18)-hexaen-13-one (100 mg, 264 umol, Intermediate O) and 3-[5-(2,7-diazaspiro[3.5]nonan-7-yl)-3-methyl-2-oxo-benzimidazol-1-yl]piperidine-2,6-dione (166 mg, 396 umol, HCl, Intermediate P) in DMSO (2 mL) was added DIEA (171 mg, 1.32 mmol). The mixture was stirred at 100° C. for 12 hrs. On completion, the reaction mixture was filtered and concentrated under reduced pressure to give the crude product. The crude product was purified by reversed-phase HPLC (0.1% FA condition) to give the title compound (90 mg, 42% yield FA) as a yellow solid. ¹H NMR (400 MHz, DMSO-d₆) δ=11.07 (br s, 1H), 9.16 (br d, J=8.8 Hz, 1H), 9.04 (s, 1H), 8.48-8.42 (m, 1H), 8.16 (d, J=9.2 Hz, 1H), 8.12-8.03 (m, 2H), 7.96 (d, J=8.8 Hz, 1H), 7.17-7.07 (m, 1H), 6.96 (d, J=8.4 Hz, 1H), 6.89 (s, 1H), 6.69 (br d, J=8.4 Hz, 1H), 6.56 (d, J=8.4 Hz, 1H), 5.31 (br dd, J=5.6, 12.8 Hz, 1H), 3.85 (s, 4H), 3.66-3.57 (m, 1H), 3.48 (br s, 2H), 3.33 (s, 3H), 3.13 (br s, 4H), 2.97-2.85 (m, 1H), 2.73-2.59 (m, 2H), 2.04-1.90 (m, 5H), 1.25-1.17 (m, 3H). LC-MS (ESI⁺) m/z 742.5 (M+H)⁺.

### Example 4. MK2 Degradation Protocol

MK2 Degradation in Human THP-1 Monoctyic Cells

The human monocytic cancer cell line THP-1 (ATCC (TIB-202)) was cultured in 96 well plates (Corning 3894) at a density of 200,000 cells per well in 100 microlitres in complete growth medium of RPMI 1640 (Gibco) pus 10% fetal Bovine Serum (Corning) and Penicillin/Streptomycin (SolarBio) at 37 degrees Celsius and 5% CO₂. A serial dilution of each compound (usually 3 to 5 fold serial dilutions up to 10 separate concentration points) was added to individual wells from a 10 millimolar stock solution, at a background concentration of 0.1% DMSO and the cells were shaken at 600 rpm for 5 minutes and then were cultured for 24 hours at 37 degrees Celsius and 5% carbon dioxide. Separately, a custom MSD (MesoScaleDiscovery, L15XA-3) plate for detection of human MK2 was prepared by coating the plate with the commercial AbCam ab247272 reagent in 1×Phosphate Buffered Saline at 40 microlitres per well and incubated at 4 degrees Celsius overnight. The next day, the MSD plate was washed with 150 microlitres of Tween buffered saline, blocked with MSD blocking buffer (3% Blocker A from MSD), and washed again.

The cell plate was centrifuged at 3,200 g for 5 minutes and the supernatant was discarded and 100 microlitres of RIPA lysis buffer (containing proteinase inhibitors, Boston BioProducts BP-115D) was added and the plate was sealed and shaken on ice for 20 minutes. The plate was centrifuged as before and 70 microlitres of sample lysate was added to the MSD plates and they were shaken at room temperature for 1 hour at 600 rpm. The MSD plate was washed 3 times as before and then the primary MK2 detection antibody (Santa Cruz sc-100393) was added at a final concentration of 1 microgram per ml at 25 microlitres per well and shaken at room temperature for 1 hour at 600 rpm.

After 3 more washes, the secondary detection antibody (SULFO-Tag anti rabbit antibody R₃₂AB-5) was added at a final concentration of 1 microgram per ml at 25 microlitres per well and shaken at room temperature for 1 hour at 600 rpm. After 3 more washes, the 1×MSD (diluted from 5× concentrate with water) reading buffer was added at 150 microlitres per well and read on the MSD instrument Meso Sector S 600.

The inhibition/degradation curves and DC₅₀ values were plotted and calculated using XL Fit (version v5.3.1.3) using equation 201 and a 4 parameter logistic model whereby fit=(A+((B−A)/(1+((x/C)D))) where A is the Bottom and B is the Top and C is the IC₅₀ and D is the slope and high C is the signal for the DMSO treated cells and Low C is the signal is the signal for the lysis buffer.

MK2 Degradation in Human SW1353 Cells

The human chondrocyte cancer cell line SW-1353 (ATCC(HTB-94)) was cultured in 96 well plates (Corning 3894) at a density of 200,000 cells per well in 100 microlitres in complete growth medium of Leibovitz's L-15 Medium (Gibco) pus 10% fetal Bovine Serum (Corning) and Penicillin/Streptomycin (SolarBio) at 37 degrees Celsius. A serial dilution of each compound (usually 3 to 5 fold serial dilutions up to 10 separate concentration points) was added to individual wells from a 10 millimolar stock solution, at a background concentration of 0.1% DMSO and the cells were shaken at 600 rpm for 5 minutes and then were cultured for 24 hours at 37 degrees Celsius. Separately, a custom MSD (MesoScaleDiscovery, L15XA-3) plate for detection of human MK2 was prepared by coating the plate with the commercial AbCam ab247272 reagent in 1×Phosphate Buffered Saline at 40 microlitres per well and incubated at 4 degrees Celsius overnight. The next day, the MSD plate was washed with 150 microlitres of Tween buffered saline, blocked with MSD blocking buffer (3% Blocker A from MSD), and washed again.

The cell plate was centrifuged at 3,200 g for 5 minutes and the supernatant was discarded and 100 microlitres of RIPA lysis buffer (containing proteinase inhibitors, Boston BioProducts BP-115D) was added and the plate was sealed and shaken on ice for 20 minutes. The plate was centrifuged as before and 70 microlitres of sample lysate was added to the MSD plates and they were shaken at room temperature for 1 hour at 600 rpm. The MSD plate was washed 3 times as before and then the primary MK2 detection antibody (Santa Cruz sc-100393) was added at a final concentration of 1 microgram per ml at 25 microlitres per well and shaken at room temperature for 1 hour at 600 rpm.

After 3 more washes, the secondary detection antibody (SULFO-Tag anti rabbit antibody R32AB-5) was added at a final concentration of 1 microgram per ml at 25 microlitres per well and shaken at room temperature for 1 hour at 600 rpm. After 3 more washes, the 1×MSD (diluted from 5× concentrate with water) reading buffer was added at 150 microlitres per well and read on the MSD instrument Meso Sector S 600.

The inhibition/degradation curves and DC₅₀ values were plotted and calculated using XL Fit (version v5.3.1.3) using equation 201 and a 4 parameter logistic model whereby fit=(A+((B−A)/(1+((x/C)D))) where A is the Bottom and B is the Top and C is the IC₅₀ and D is the slope and high C is the signal for the DMSO treated cells and Low C is the signal is the signal for the lysis buffer.

MK2 degradation results for compounds of the invention are presented in Table 4. The letter codes for MK2 DC₅₀ include: A (<0.001 μM), B (0.001-0.01 μM), C (0.01-0.1 μM), and D (>0.1 μM). The letter codes of MK2 Dmax include A (>75%), B (50-75%), and C (>50%). “-”=not tested or inactive.

TABLE 4
MK2 Degradation Results

| | MK2 MSD | | MK2 MSD | |
|---|---|---|---|---|
| | THP-1 | | SW1353 | |
| | degradation | | degradation | |
| | 24 h | | 24 h | |
| | GMean | MK2 MSD | GMean | MK2 MSD |
| | External | THP-1 24 h | External | SW 1353 |
| I-# | Abs DC₅₀ | Dmax % | Abs DC₅₀ | 24 h Dmax % |
| I-1 | D | C | D | C |
| I-2 | D | C | D | C |
| I-34 | A | A | A | A |
| I-36 | B | A | B | A |
| I-43 | A | A | A | A |
| I-45 | C | A | C | A |
| I-48 | C | A | B | A |
| I-49 | B | A | B | A |
| I-50 | B | A | B | A |
| I-51 | B | A | B | A |
| I-53 | C | B | C | A |
| I-54 | C | B | C | A |
| I-55 | B | A | B | A |
| I-54 | C | A | B | A |
| I-86 | B | A | B | A |
| I-87 | B | A | B | A |
| I-88 | B | A | B | A |
| I-92 | B | A | B | A |
| I-93 | A | A | B | A |
| I-94 | A | A | B | A |
| I-95 | A | A | B | A |
| I-96 | A | A | B | A |
| I-98 | A | A | A | A |
| I-99 | A | A | A | A |
| I-100 | B | A | B | A |
| I-103 | A | A | B | A |
| I-104 | B | A | C | A |
| I-105 | A | A | B | A |
| I-106 | A | A | B | A |
| I-107 | A | A | B | A |
| I-118 | A | A | A | A |
| I-119 | A | A | A | A |
| I-120 | B | A | B | A |
| I-121 | D | C | D | C |
| I-122 | A | A | A | A |
| I-125 | A | A | A | A |
| I-126 | B | A | B | A |
| I-127 | B | A | B | A |
| I-128 | B | A | B | A |
| I-129 | A | A | A | A |
| I-130 | A | A | A | A |
| I-131 | A | A | A | A |
| I-133 | D | C | D | C |
| I-134 | D | C | D | C |
| I-135 | D | C | D | C |
| I-137 | B | A | A | A |
| I-138 | A | A | A | A |
| I-139 | A | A | B | A |
| I-141 | A | A | A | A |
| I-143 | A | A | A | A |
| I-146 | D | C | D | C |
| I-154 | C | A | C | A |
| I-155 | D | C | D | C |
| I-156 | A | A | A | A |
| I-157 | A | A | A | A |
| I-158 | A | A | B | A |
| I-160 | A | A | A | A |
| I-161 | A | A | A | A |
| I-165 | A | A | A | A |
| I-167 | D | C | D | C |
| I-168 | D | C | D | C |
| I-170 | B | A | B | A |
| I-171 | B | B | B | A |
| I-172 | A | A | A | A |
| I-173 | A | A | A | A |
| I-174 | A | A | A | A |
| I-175 | B | A | B | A |
| I-176 | B | B | B | A |
| I-177 | C | A | C | A |
| I-178 | C | A | C | A |
| I-179 | — | A | — | A |
| I-180 | — | A | — | A |
| I-181 | — | A | — | A |
| I-182 | — | C | — | C |
| I-183 | — | A | — | A |
| I-184 | — | A | — | A |
| I-185 | — | A | — | A |
| I-186 | — | A | — | A |
| I-187 | D | C | D | C |
| I-188 | B | A | B | A |
| I-189 | A | A | A | A |
| I-190 | A | A | A | A |
| I-191 | C | B | B | A |
| I-192 | A | A | A | A |
| I-193 | D | C | D | — |
| I-194 | A | A | A | A |
| I-195 | A | A | A | A |
| I-196 | B | A | — | — |
| I-197 | C | A | — | — |
| I-198 | C | A | — | — |
| I-199 | B | A | — | A |
| I-200 | B | A | — | A |
| I-201 | D | C | — | — |
| I-202 | D | C | — | — |
| I-203 | B | A | — | A |
| I-204 | B | A | — | — |
| I-205 | A | A | — | — |
| I-206 | B | B | — | — |
| I-207 | B | B | — | A |
| I-208 | D | C | D | C |
| I-209 | D | C | — | C |
| I-210 | C | B | C | A |
| I-211 | C | B | C | A |
| I-212 | C | A | C | A |
| I-213 | A | A | A | A |
| I-214 | B | B | A | A |
| I-215 | — | B | A | A |
| I-216 | A | A | B | A |
| I-217 | D | C | D | C |
| I-218 | A | A | A | A |
| I-219 | D | C | D | C |
| I-220 | D | C | D | C |
| I-221 | A | A | A | A |
| I-222 | A | A | A | A |
| I-224 | D | C | D | C |
| I-225 | D | C | D | C |
| I-226 | A | A | A | A |
| I-229 | D | C | D | C |
| I-230 | A | A | A | A |
| I-231 | B | A | B | A |
| I-232 | A | A | A | A |
| I-233 | B | A | B | A |
| I-234 | A | A | A | A |
| I-235 | A | A | A | A |
| I-236 | A | A | A | A |
| I-237 | A | A | A | A |
| I-238 | B | A | B | A |
| I-239 | A | A | A | A |
| I-240 | A | A | A | A |
| I-241 | B | B | B | A |
| I-242 | C | B | B | A |
| I-243 | A | B | A | A |
| I-244 | A | A | A | A |
| I-245 | A | A | A | A |
| I-246 | A | A | A | A |
| I-247 | B | A | A | A |
| I-248 | B | A | B | A |
| I-249 | B | B | A | B |
| I-250 | A | B | A | A |
| I-251 | — | C | A | B |
| I-252 | A | A | A | A |
| I-253 | B | A | B | A |
| I-254 | A | A | A | A |
| I-255 | B | A | B | A |
| I-256 | A | A | A | A |
| I-257 | D | C | D | C |
| I-258 | B | C | B | B |
| I-259 | — | C | B | A |
| I-260 | — | B | A | A |
| I-261 | D | C | C | A |
| I-262 | A | A | A | A |
| I-263 | A | A | A | A |
| I-264 | B | A | B | A |
| I-265 | B | A | B | A |
| I-266 | B | A | B | A |
| I-267 | D | C | D | C |
| I-268 | B | A | B | A |
| I-269 | A | A | B | A |
| I-270 | A | A | B | A |
| I-271 | A | A | A | A |
| I-272 | A | A | A | A |
| I-274 | C | A | C | A |
| I-275 | A | A | A | A |
| I-276 | B | A | B | A |
| I-277 | A | A | A | A |
| I-278 | A | A | A | A |
| I-279 | A | A | A | A |
| I-280 | A | A | A | A |
| I-281 | A | A | B | A |
| I-282 | A | A | A | A |
| I-283 | A | A | A | A |
| I-284 | B | A | B | A |
| I-285 | B | A | B | A |
| I-286 | C | B | C | A |
| I-287 | A | A | A | A |
| I-288 | A | A | A | A |
| I-289 | A | A | B | A |
| I-290 | A | A | B | A |
| I-291 | A | A | A | A |
| I-292 | A | A | B | A |
| I-293 | A | A | C | A |
| I-294 | A | A | B | A |
| I-295 | C | B | C | A |
| I-296 | B | — | C | — |
| I-297 | A | A | A | A |
| I-298 | A | A | A | A |
| I-299 | A | A | A | A |
| I-300 | D | C | D | C |
| I-301 | D | C | D | C |
| I-302 | B | A | B | A |
| I-303 | A | A | B | A |
| I-304 | B | A | B | A |
| I-305 | A | A | A | A |
| I-306 | A | A | A | A |
| I-307 | B | A | B | A |
| I-308 | A | A | A | A |
| I-309 | D | C | D | — |
| I-310 | B | A | B | A |
| I-311 | B | A | B | A |
| I-312 | B | A | B | A |
| I-313 | B | A | B | A |
| I-314 | A | A | A | A |
| I-315 | B | A | B | A |
| I-316 | D | C | D | C |
| I-317 | A | A | B | A |
| I-318 | D | C | D | C |
| I-389 | D | C | D | C |
| I-390 | D | C | D | C |
| I-391 | A | A | A | A |
| I-393 | D | C | D | C |
| I-395 | B | A | A | A |
| I-402 | B | B | A | A |
| I-403 | A | A | A | A |
| I-404 | A | A | A | A |
| I-405 | A | A | A | A |
| I-406 | D | C | D | C |
| I-407 | D | C | D | — |
| I-408 | D | C | D | — |
| I-409 | A | A | A | A |
| I-410 | A | A | A | A |
| I-411 | C | B | B | B |
| I-412 | A | A | A | A |
| I-413 | A | A | B | A |
| I-414 | B | A | A | A |
| I-415 | A | A | A | A |
| I-416 | D | C | C | B |
| I-417 | B | A | A | A |
| I-418 | B | A | A | A |
| I-419 | A | A | A | A |
| I-420 | B | A | B | A |
| I-421 | B | A | B | A |
| I-422 | A | A | A | A |
| I-423 | A | A | A | A |
| I-424 | B | A | A | A |
| I-425 | A | A | A | A |
| I-426 | A | A | A | A |
| I-427 | A | A | A | A |
| I-428 | B | A | A | A |
| I-429 | D | C | D | C |
| I-430 | A | A | A | A |
| I-431 | D | C | C | B |
| I-432 | C | B | A | A |
| I-433 | D | C | D | C |
| I-434 | D | C | D | C |
| I-435 | D | C | D | — |
| I-436 | D | C | D | — |
| I-437 | B | A | B | A |
| I-438 | B | A | B | A |
| I-439 | B | A | A | A |
| I-440 | A | A | A | A |
| I-441 | B | A | A | A |
| I-442 | D | C | D | C |
| I-443 | A | A | A | A |
| I-444 | B | B | B | A |
| I-445 | A | A | A | A |
| I-446 | D | C | D | C |
| I-447 | D | C | C | B |
| I-448 | D | C | B | B |
| I-449 | D | C | D | C |
| I-450 | D | C | D | C |
| I-451 | A | A | A | A |
| I-452 | C | B | B | B |
| I-453 | D | C | D | C |
| I-454 | D | C | D | C |
| I-458 | D | C | D | C |
| I-459 | D | C | D | C |
| I-460 | D | C | D | C |
| I-461 | C | B | B | A |
| I-462 | B | A | C | A |
| I-463 | B | A | B | A |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1-11. (canceled)

12. A compound of formula I-dd-1: 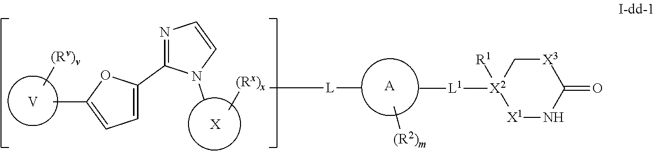
or a pharmaceutically acceptable salt, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or 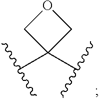 [SMILES: *C1(*)COC1]
X² is a carbon atom or silicon atom;
X³ is a bivalent moiety selected from —CR₂—, —NR—, —O—, —S—, or —Si(R²)—;
R¹ is hydrogen, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form an optionally substituted 3-11 membered saturated or partially unsaturated monocyclic, bicyclic, bridged bicyclic, or spirocyclic carbocyclic ring or heterocyclic ring with 1-4 heteroatoms independently selected from nitrogen, oxygen, and sulfur;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a bi- or tricyclic ring selected from 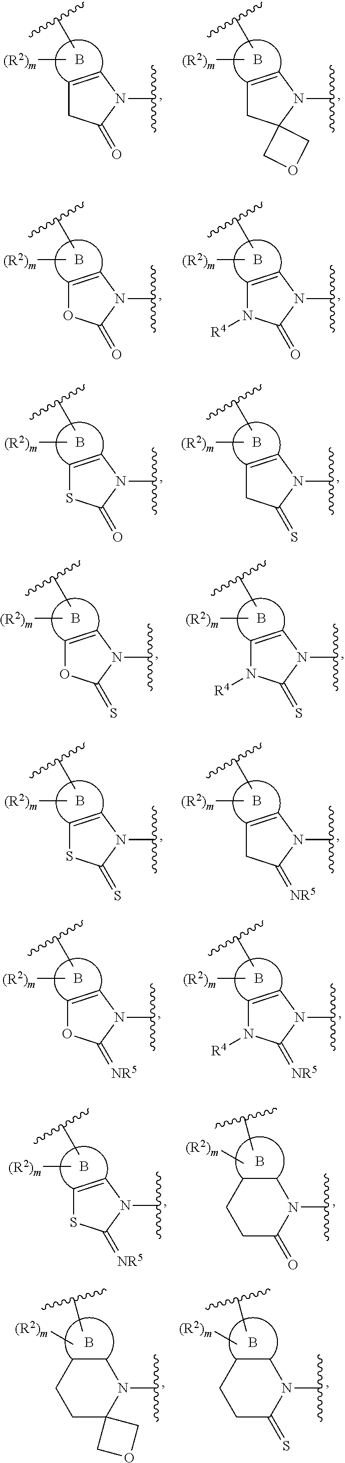 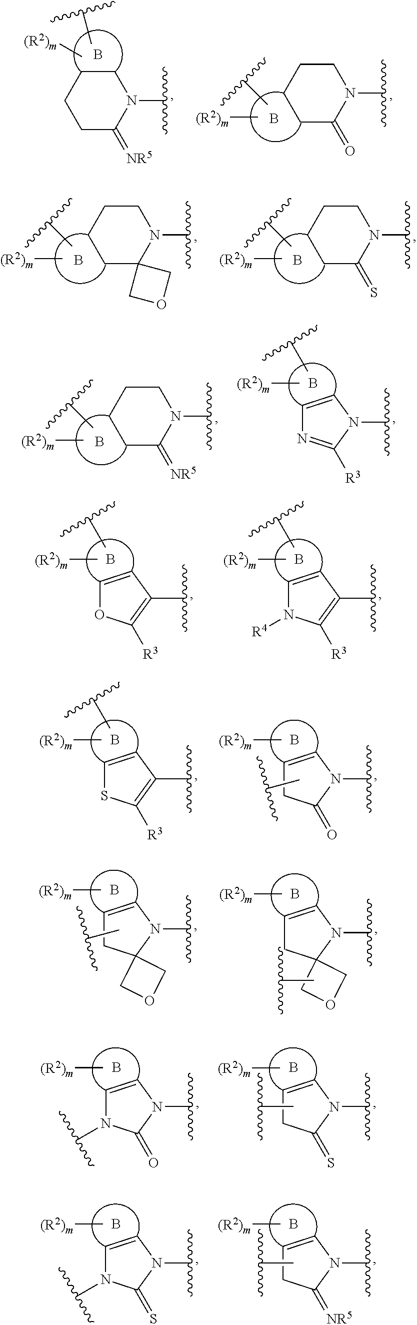 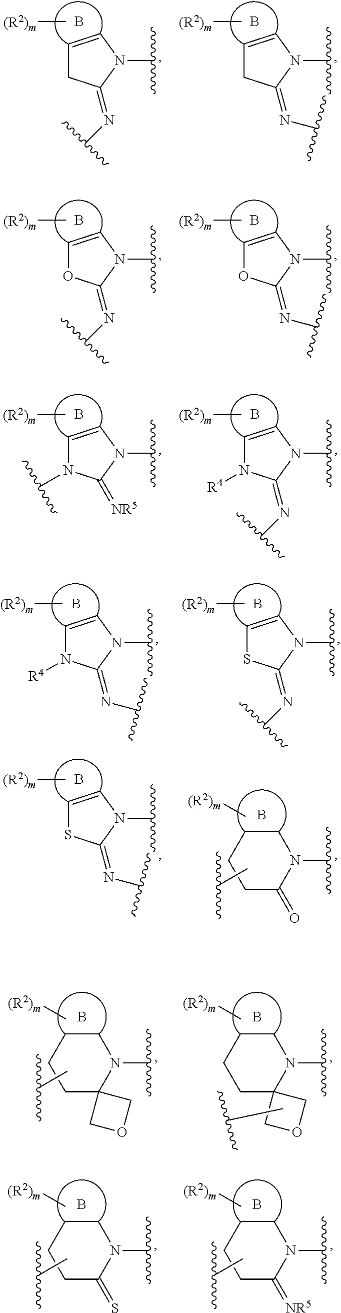 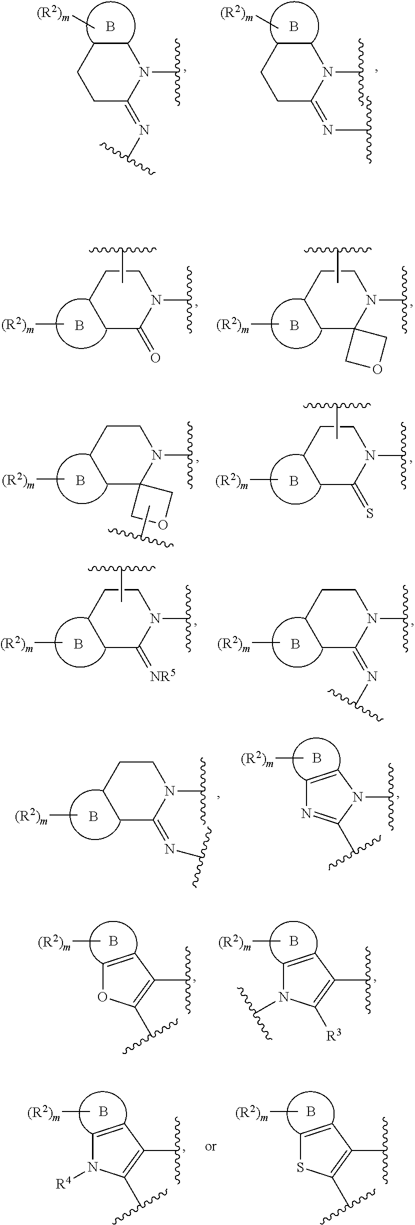
wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R⁴ is independently hydrogen, R⁶, halogen, —CN, —NO₂, —OR, SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
L is a covalent bond or a bivalent, saturated or partially unsaturated, straight or branched C₁₋₅₀ hydrocarbon chain, wherein 0-10 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, 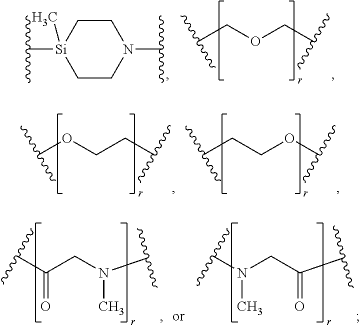
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
Ring V and Ring X are, independently, a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicyclic, or spirocyclic carbocyclyl or heterocyclyl with 1-4 heteroatoms independently selected from nitrogen, oxygen, and sulfur;
R^{v} and R^{x} are, independently, hydrogen, R^{A}, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)NROR, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
v is 0, 1, 2, 3, or 4; and
x is 0, 1, 2, 3, or 4.

13-14. (canceled)

15. The compound of claim 12, wherein Ring A is

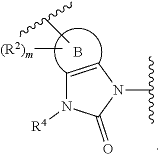

16. The compound of claim 12, wherein Ring B is benzo.

17. The compound of claim 12, wherein R⁴ is methyl.

18-27. (canceled)

28. The compound of claim 12, wherein L is a covalent bond or a bivalent, saturated or partially unsaturated, straight or branched C₁₋₂₀ hydrocarbon chain, wherein 0-6 methylene units of L are independently replaced by -Cy-, —O—, —N(R)—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, or —N(R)C(O)O—.

29. The compound of claim 12, wherein said compound is selected from: 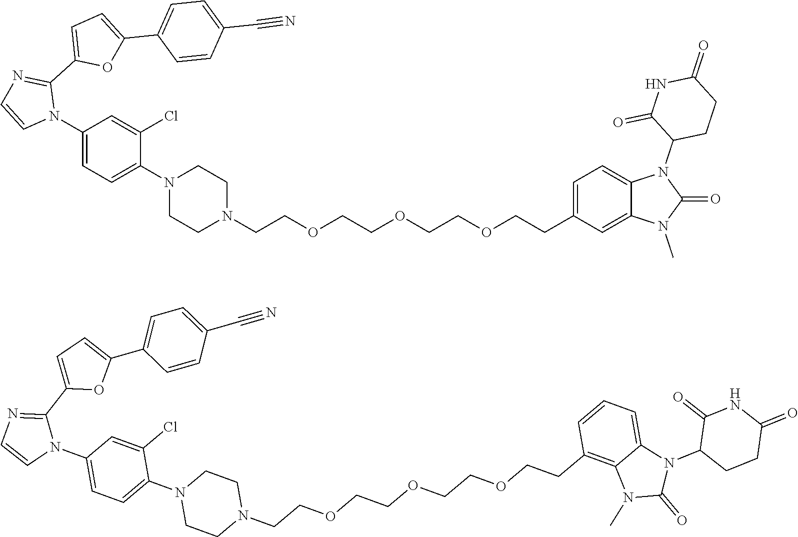 [SMILES: Cn1c(=O)n(C2CCC(=O)NC2=O)c2ccc(CCOCCOCCOCCN3CCN(c4ccc(-n5ccnc5-c5ccc(-c6ccc(C#N)cc6)o5)cc4Cl)CC3)cc21 ; Cn1c(=O)n(C2CCC(=O)NC2=O)c2cccc(CCOCCOCCOCCN3CCN(c4ccc(-n5ccnc5-c5ccc(-c6ccc(C#N)cc6)o5)cc4Cl)CC3)c21]
or a pharmaceutically acceptable salt thereof.

30. A pharmaceutical composition comprising a compound of claim 12, and a pharmaceutically acceptable carrier, adjuvant, or vehicle.

31. The pharmaceutical composition according to claim 30, further comprising an additional therapeutic agent.

32. A method of degrading MK2 protein in a patient or biological sample comprising administering to said patient, or contacting said biological sample with a compound of claim 12, or a pharmaceutical composition thereof.

33. A method of treating an MK2-mediated disorder, disease, or condition in a patient comprising administering to said patient a compound of claim 12, or a pharmaceutical composition thereof.

34. The method of claim 33, further comprising administration of an additional therapeutic agent.

35. The method of claim 33, wherein the MK2-mediated disorder, disease or condition is selected from connective tissue and joint disorders, neoplasia disorders, cardiovascular disorders, otic disorders, ophthalmic disorders, respiratory disorders, gastrointestinal disorders, angiogenesis-related disorders, immunological disorders, inflammatory disorders, allergic disorders, nutritional disorders, infectious diseases and disorders, endocrine disorders, metabolic disorders, neurological and neurodegenerative disorders, psychiatric disorders, hepatic and biliary disorders, musculoskeletal disorders, genitourinary disorders, gynecologic and obstetric disorders, injury and trauma disorders, surgical disorders, dental and oral disorders, sexual dysfunction disorders, dermatologic disorders, hematological disorders, and poisoning disorders.

36. The compound of claim 12, wherein R^{x} is —N(R)₂.

37. The compound of claim 12, wherein L is

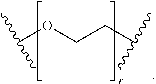

38. The compound of claim 37, wherein r is 3.

39. The compound of claim 12, wherein V is phenyl.

40. The compound of claim 12, wherein R^{v} is halogen.

41. The compound of claim 12, wherein R^{v} is —CN.

42. The compound of claim 12, wherein Ring V is phenyl or a 5-10 membered heteroaryl containing 1-4 heteroatoms independently selected from nitrogen, oxygen, and sulfur.

42. The compound of claim 12, wherein Ring X is phenyl or a 5-10 membered heteroaryl containing 1-4 heteroatoms independently selected from nitrogen, oxygen, and sulfur.
